(19) United States
(12) Patent Application Publication
(10) Pub. No.: US 2025/0114460 A1
(43) Pub. Date: Apr. 10, 2025

(54) NOVEL PLK1 DEGRADATION INDUCING COMPOUND

(71) Applicant: UPPTHERA, INC., Incheon (KR)

(72) Inventors: Soo Hee Ryu, Incheon (KR); Im Suk Min, Gyeonggi-do (KR); Han Kyu Lee, Gyeonggi-do (KR); Seong Hoon Kim, Incheon (KR); Hye Guk Ryu, Incheon (KR); Keum Young Kang, Incheon (KR); Sang Youn Kim, Incheon (KR); So Hyun Chung, Incheon (KR); Jun Kyu Lee, Gyeonggi-do (KR); Gibbeum Lee, Gyeonggi-do (KR)

(73) Assignee: UPPTHERA, INC., Incheon (KR)

(21) Appl. No.: 18/580,553

(22) PCT Filed: Aug. 10, 2022
(86) PCT No.: PCT/IB2022/057471

§ 371 Date: Jan. 18, 2024

(30) Foreign Application Priority Data
Aug. 10, 2021 (KR) 10-2021-0105358
Aug. 12, 2021 (KR) 10-2021-0106488
Sep. 3, 2021 (KR) 10-2021-0117389
Sep. 24, 2021 (KR) 10-2021-0126757
Jan. 20, 2022 (KR) 10-2022-0008456
Feb. 17, 2022 (KR) 10-2022-0020996
May 3, 2022 (KR) 10-2022-0054880
Jun. 21, 2022 (KR) 10-2022-0075838

(51) Int. Cl. A61K 47/55

(52) U.S. Cl. CPC A61K 47/55

(57) ABSTRACT

The present disclosure relates to a novel PLK1 degradation inducing compound, a method for preparing the same, and the use thereof. The compounds of the present disclosure exhibit an effect of inducing PLK1 degradation. Therefore, the compounds of the present disclosure may be effectively utilized for preventing or treating PLK1-related diseases.

### TECHNICAL FIELD

The present disclosure relates to a novel PLK1 degradation inducing compound, a method for preparing the same, and the use thereof. It can specifically act on abnormal cells, etc. and can be usefully used in the treatment of various diseases through efficient degradation of PLK1.

### BACKGROUND ART

Polo-like kinase 1 (PLK1) is a serine/threonine kinase involved in the conversion of G2/M phase during cell growth and division. PLK1 is expressed and activated in a pulse form from the S phase to the G2/M phase, and rapidly degrades as mitosis ends.

PLK1 is overexpressed in various carcinomas such as colon cancer, lung cancer, bladder cancer, and melanoma, etc., and cancer cells overexpressing PLK1 tend to show resistance to various types of anticancer drugs. As the PLK1 dependence in various carcinomas was revealed as described above, there have been attempts to develop PLK1 inhibitor compounds such as volasertib (also known as BI6727), etc.

However, the conventional PLK1 inhibitors do not sufficiently inhibit PLK1 activity at concentrations that are clinically safe. Thus, there is a problem that even if the cell cycle of cancer cells is temporarily delayed, some cancer cells eventually restart the cell cycle, which may not obtain sufficient clinical effects (see Gheghiani et al., Cell Reports, 2017, etc.). In fact, many pharmaceutical companies such as Boehringer Ingelheim, GlaxoSmithKline, etc., have attempted to develop small-molecular compound-based PLK1 inhibitors, but most of them have failed or stopped in the clinical trial stage, and thus there are no commercially available PLK1 inhibitors to date. It shows that pharmacological mechanism that follows the method of inhibiting enzyme activity by binding to the active site of PLK1 like the small molecule compound inhibitors is not sufficiently effective in the development of new drugs intended to derive anticancer effects by inhibiting PLK1 activity of cancer cells.

Recently, a proteolysis targeting chimera (PROTAC) has been proposed as a small molecule-based platform technology capable of inducing proteolysis of a target protein in the body. The PROTAC is a bifunctional compound in which a ligand molecule that binds to disease-related target protein and an E3 ubiquitin ligase binding moiety are linked by a chemical linker. Theoretically, the PROTAC compound is capable of inducing degradation of the target protein by placing the disease-related target protein near the E3 ubiquitin ligase. Based on this new mechanism different from the existing inhibitors, a lot of PROTAC compounds have been developed as therapeutic agents for cancer and inflammatory diseases, etc., and being studied with various extensibility (e.g. as payloads of ADC (Antibody-Drug Conjugates)). However, it does not show activity in all ranges of binding moieties or linkers, and in order for PROTAC to exhibit the desired level of efficacy, it is known through several studies that each binding moiety and linker must have an appropriately linked structure (see US2020-0325130A). In particular, in the case of the CRBN(Cereblon) E3 ligase targeting moiety, depending on the type of the binding moiety or the structure of the compound linked thereto, there is a risk of degrading CRBN neo-substrate (GSPT1, IKZF1/3, etc.) or showing off-target toxicity accordingly. Therefore, it is important to select appropriate binding moieties and optimize the structure of the entire compound so as not to exhibit unexpected toxicity during PROTAC drug development.

In the case of the PROTAC compound using PLK1 as a target protein, Chinese Patent Laid-Open No. 106543185 A discloses some bifunctional compounds in which a volasertib derivative compound and a binding moiety for the E3 ubiquitin ligase CRBN are linked by a chemical linker. However, the related art document merely describes some limited forms of synthesis examples of PROTAC compounds, wherein in general, the target degradation activity and selectivity of PROTAC may vary significantly depending on selection of the target protein moiety, the E3 ubiquitin ligase binding moiety, and the like (see Burslem and Crews, 2017, etc.).

Further, the PROTAC compound described in the above-described document is characterized by a compound that simultaneously degrades PLK1 and BRD4, and degrade various proteins such as other PLK family proteins and BRD4, etc.), which may cause side effects due to off-target toxicities at the time of drug development. In particular, it is known that strong inhibition of BRD4 activity inevitably accompanies on-target toxicity such as blood toxicity and gastrointestinal toxicity along with pharmacological effects. Therefore, the PROTAC compound described in the above document would expect to face greater clinical side effects as more BRD4 protein gets degraded (see Bolden et al. Cell Reports, 2014).

Moreover, according to the document published by the inventors of the above document (see Mu et al. BBRC, 2019), it can be confirmed that the PROTAC compound, which simultaneously degrades PLK1 and BRD4, has much stronger BRD4 degradation ability than PLK1 degradation ability at the cellular level, and the cell cycle thereof almost stops in the G1 phase, etc., that is, the PROTAC compound actually acts only as a BRD4 inhibitor regardless of the way that the conventional PLK1 inhibitors exert pharmacological effects.

Therefore, there is an unsatisfied demand for effective PLK1 degradation inducing compound with no or minimal side effects. (e.g. off-target toxicity)

### DISCLOSURE OF INVENTION

### Technical Problem

An object of the present disclosure is to provide novel PLK1 degradation inducing compounds.

Another object of the present disclosure is to provide a method for preparing the compounds.

Still another object of the present disclosure is to provide a use of the compounds.

### Solution to Problem

In order to achieve the above-described objects, the present inventors made efforts to study, and as a result, found that novel PROTAC compounds of the present invention specifically act on abnormal cells overexpressing PLK1 through appropriate structural combination and optimization of E3 Ligase binder, Target binding moiety, and Linker to induce effective PLK1 degradation and minimize side effects, and completed the present invention.

### Selective PLK1 Degradation Inducing Compounds

The present disclosure provides novel compounds that induce effective polo-like kinase 1 (PLK1) degradation. Specifically, the present disclosure provides a bifunctional compound in which a PLK1 binding moiety and an E3 ubiquitin ligase-binding moiety are linked by a chemical linker.

In one general aspect, there is provided a compound represented by the following Formula I, a steroisomer thereof or a pharmaceutically acceptable salt thereof:

ULM-Linker-PTM [Formula I]
in the Formula I above,
ULM is a moiety represented by the following Formula 1;

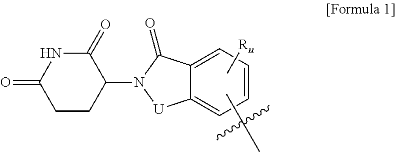

PTM is a moiety represented by the following Formula 2;

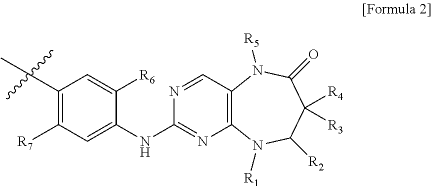

Linker is a group that chemically links ULM and PTM;
U is —CH₂— or —C(═O)—;
R_{U} is —H or -halo;
R₁ is —C₁₋₄alkyl or 3- to 7-membered cycloalkyl;
R₂ is —H;
R₃ and R₄ are each independently —H, —C₁₋₄alkyl, —C₁₋₄alkenyl or -halo, or R₃ and R₄ are linked each other to form a 3- to 6-membered ring;
R₅ is —C₁₋₄alkyl;
R₆ is —C₁₋₄alkyl, —C₁₋₄haloalkyl, —O—R_{P} or -halo;
R₇ is —H, —C₁₋₄alkyl, —C₁₋₄haloalkyl, -halo or 5- to 6-membered heterocycloalkyl {wherein at least one H of the 5- to 6-membered heterocycloalkyl ring may be substituted —C₁₋₄alkyl}, or is linked with the Linker to form a 5- to 6-membered ring; and
R_{P} is —H, —C₁₋₄alkyl, —C₁₋₄hydroxyalkyl or —C₁₋₄haloalkyl.

In one embodiment of the present disclosure,
ULM is a moiety represented by following Formula 1-1 or Formula 1-2;

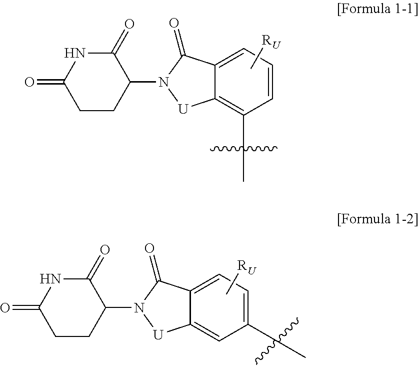

U is —CH₂— or —C(═O)—; and
R_{U} is —H or -halo.

In one embodiment of the present disclosure,
ULM is

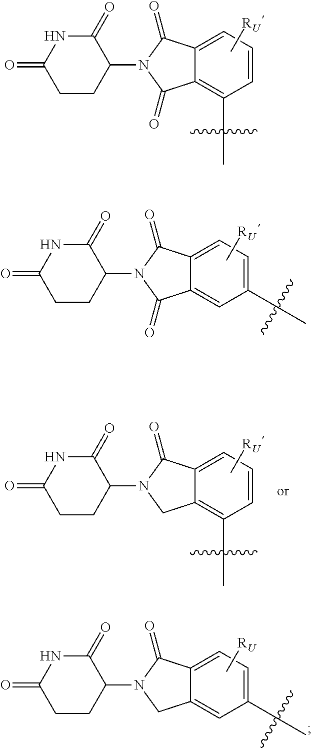

and
R_{U} is —H or -halo.

In one embodiment of the present disclosure,
PTM is

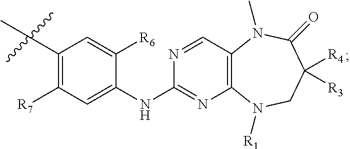

R₁ is -isopropyl, -cyclopropyl, -cyclobutyl or -cyclopentyl;
R₃ and R₄ are each independently —H, —C₁₋₄alkyl, —C₁₋₄alkenyl or -halo, or R₃ and R₄ are linked each other to form a 3-membered ring;
R₆ is —C₁₋₄alkyl, —C₁₋₄haloalkyl, —O—R_{P} or -halo;
R₇ is —H, —C₁₋₄haloalkyl, -halo or 5- to 6-membered heterocycloalkyl {wherein at least one H of the 5- to 6-membered heterocycloalkyl may be substituted —C₁₋₄alkyl}, or is linked with the Linker to form a 5- to 6-membered heterocycloalkyl ring; and
R_{P} is —H, —C₁₋₄alkyl, —C₁₋₄hydroxyalkyl or —C₁₋₄haloalkyl.

In one embodiment of the present disclosure,
Linker is -L_{U}-L₁-L₂-L₃-L_{P}-;
L_{U} is —(CH₂)x-, —C≡C—, —NH—, —O—, —S— or nothing (null) {wherein L_{U} is linked with ULM [wherein, when the L_{U} is nothing (null), L₁ is directly linked with ULM], and the x is 0, 1, 2, 3 or 4};
L₁ is heterocycloalkyl or nothing (null) {wherein, when the L₁ is nothing (null), L_{U} and L₂ are directly linked, the heterocycloalkyl contains at least one N atom in the ring, and at least one H of the heterocycloalkyl ring may be substituted with —C₁₋₄alkyl, —C₁₋₄ haloalkyl, —C₁₋₄alkoxy, —OH, -halo or ═O};
L₂ is —(CH₂)y₁-, —(CH₂)y₂-C(═O)—(CH₂)y₃-, —C(═O)—(CH₂)y₁-C(═O)—, —(CH₂)y₂-O—(CH₂)y₃-C(═O)—, —(CH₂)y₂-NH—(CH₂)y₃-, —(CH₂)y₂-N(C₁₋₄alkyl)-(CH₂)y₃-, —(CH₂)y₂-O—(CH₂)y₃-, —(CH₂)y₁-(O—C₁₋₄alkyl)z-O—C₁₋₄alkyl-, —(CH₂)y₂-C₁₋₄alkenyl-(CH₂)y₃- or —(CH₂)y₂-phenyl-(CH₂)y₃- {wherein the y₁ to y₃ are each independently 0, 1, 2, 3, 4, 5 or 6, and the z is 1, 2, 3, 4, 5 or 6};
L₃ is cycloalkyl, heterocycloalkyl, phenyl or nothing (null) {wherein, when the L₃ is nothing (null), L₂ and L_{P} are directly linked, the heterocycloalkyl contains at least one N atom in the ring, and at least one H of the cycloalkyl, heterocycloalkyl or phenyl ring may be substituted with —C₁₋₄alkyl, —C₁₋₄haloalkyl or -halo};
L_{P} is —(CH₂)z-NR_{L}—C(═O)—, -cycloalkyl-NH—C(═O)—, -heterocycloalkyl-NH—C(═O)—, —C(═O)—, —(CH₂)z-O— or nothing (null) {wherein —(C═O)— or —O— of the L_{P} is linked with PTM [wherein, when the L_{P} is nothing (null), cycloalkyl or heterocycloalkyl of L₃ is directly linked with PTM], NR_{L} of the —(CH₂)z-NR_{L}—C(═O)— is linked with R₇ to form a 5- to 6-membered ring, and the z is 0, 1, 2, 3 or 4}; and
R_{L} is —H or —C₁₋₄alkyl.

In one embodiment of the present disclosure,
L_{U} is —(CH₂)x-, —C≡C—, —NH—, —O—, —S— or nothing (null) {wherein L_{U} is linked with ULM [wherein, when the L_{U} is nothing (null), L₁ is directly linked with ULM], and the x is 0 or 1};
L₁ is 4- to 12-membered heterocycloalkyl or nothing (null) {wherein, when the L₁ is nothing (null), L_{U} and L₂ are directly linked, the 4- to 12-membered heterocycloalkyl is single ring, bridged bicyclic ring or spiro ring, the 4- to 12-membered heterocycloalkyl contains at least one N atom in the ring, and at least one H of the 4- to 12-membered heterocycloalkyl ring may be substituted with —OH or -halo};
L₂ is —(CH₂)y₁-, —(CH₂)y₂-C(═O)—(CH₂)y₃-, —C(═O)—(CH₂)y₁-C(═O)—, —(CH₂)y₂-O—(CH₂)y₃-C(═O)—, —(CH₂)y₂-N(C₁₋₄alkyl)-(CH₂)y₃-, —(CH₂)y₂-O—(CH₂)y₃-, —(CH₂)y₁-(O—C₁₋₄ alkyl)z-O—C₁₋₄alkyl-, —(CH₂)y₂-C₁₋₄alkenyl-(CH₂)y₃- or —(CH₂)y₂-phenyl-(CH₂)y₃-{wherein the y₁ to y₃ are each independently 0, 1, 2, 3, 4 or 5, and the z is 1, 2, 3, 4 or 5};
L₃ is 4- to 6-membered cycloalkyl, 4- to 12-membered heterocycloalkyl, phenyl or nothing (null) {wherein, when the L₃ is nothing (null), L₂ and L_{P} are directly linked, the 4- to 12-membered heterocycloalkyl is single ring, bridged bicyclic ring or spiro ring, the 4- to 12-membered heterocycloalkyl contains at least one N atom in the ring, and at least one H of the 4- to 6-membered cycloalkyl, 4- to 12-membered heterocycloalkyl or phenyl ring may be substituted with -halo};
L_{P} is —(CH₂)z-NR_{L}—C(═O)—, -cycloalkyl-NH—C(═O)—, -heterocycloalkyl-NH—C(═O)—, —C(═O)—, —(CH₂)z-O— or nothing (null) {wherein —(C═O)— or —O— of the L_{P} is linked with PTM [wherein, when the L_{P} is nothing (null), cycloalkyl or heterocycloalkyl of L₃ is directly linked with PTM], NR_{L} of the —(CH₂)z-NR_{L}—C(═O)— may be linked with R₇ to form a 5- to 6-membered heterocycloalkyl ring, and the z is 0 or 1}; and
R_{L} is —H or —C₁₋₄alkyl.

In a certain embodiment of the present disclosure, the compound represented by Formula I is a compound that is selected from the group consisting of Compound 1 to 225.

In the present disclosure, a pharmaceutically acceptable salt refers to any organic or inorganic acid addition salt with a concentration that is relatively non-toxic, is harmless, and has effective action to patients, wherein side effects caused by this salt does not deteriorate beneficial efficacy of the compound represented by Formula I. For example, the pharmaceutically acceptable salt may be an inorganic acid such as hydrochloric acid, phosphoric acid, sulfuric acid, nitric acid, or the like, or an organic acid such as methanesulfonic acid, p-toluenesulfonic acid, acetic acid, trifluoroacetic acid, maleic acid, succinic acid, oxalic acid, benzoic acid, tartaric acid, fumaric acid, manderic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid or hydroiodic acid, but is not limited thereto.

### Use of the Selective PLK1 Degradation Inducing Compounds

An embodiment of the present disclosure is a composition for inducing PLK1 degradation including a compound represented by Formula I or a pharmaceutically acceptable salt thereof. The Formula I is the same as defined above.

In the experimental examples of the present disclosure, it was confirmed that the compounds of the present disclosure effectively induce the protein degradation of PLK1.

The PLK1 degradation-inducing PROTAC compound of the present disclosure is capable of fundamentally degrading the target protein, PLK1 in view of the mechanism of action, thereby achieving an excellent PLK1 inhibitory effect as compared to the conventional PLK1 small molecule inhibitor that inhibits the simple activity of PLK1.

Accordingly, the composition including the compound represented by Formula I of the present disclosure or a pharmaceutically acceptable salt thereof may be effectively employed for selective degradation of PLK1.

An embodiment of the present disclosure is a composition for preventing or treating PLK1-related diseases including the compound represented by Formula I or the pharmaceutically acceptable salt thereof. An another embodiment of the present disclosure is a method for the prevention or treatment of PLK-related diseases comprising administering the composition to a subject in need thereof. The Formula I is the same as defined above.

In the present disclosure, the PLK1-related disease refers to any disease or condition capable of being treated, alleviated, delayed, inhibited or prevented from induction of degradation or inhibition of activity of PLK1. In an embodiment, the PLK1-related disease may be a cancer (malignant tumor), a benign tumor, a neurological disease, or other genetic or non-genetic diseases caused by excessive cell division.

The cancer includes all cancers capable of exhibiting prophylactic or therapeutic efficacy due to inhibition of PLK1 activity, and may be solid cancer or blood cancer. For example, the cancer may be one or more selected from the group consisting of squamous cell carcinoma, small cell lung cancer, non-small cell lung cancer, lung adenocarcinoma, lung squamous cell carcinoma, peritoneal cancer, skin cancer, skin or intraocular melanoma, rectal cancer, anal muscle cancer, esophageal cancer, small intestine cancer, endocrine cancer, parathyroid cancer, adrenal cancer, soft tissue sarcoma, urethral cancer, chronic or acute leukemia, lymphocytic lymphoma, hepatocellular carcinoma, gastrointestinal cancer, gastric cancer, pancreatic cancer, glioblastoma, cervical cancer, ovarian cancer, liver cancer, bladder cancer, liver tumor, breast cancer, colon cancer, colorectal cancer, endometrial or uterine cancer, salivary gland cancer, kidney cancer, prostate cancer, vulvar cancer, thyroid cancer, head and neck cancer, brain cancer, osteosarcoma, solid tumor, blood cancer, bone cancer, large cell lymphoma, adrenocorticoid tumor, t cell lymphoma/leukemia, neuroendocrine cancer, neuroendocrine tumor, cholangiocarcinoma, neuroblastoma, glioblastoma, glioma, and the like, but is not limited thereto. The cancer includes not only primary cancer but also metastatic cancer.

The benign tumors include all benign tumors capable of exhibiting prophylactic or therapeutic efficacy due to the inhibition of PLK1 activity, such as benign tumors in pre-cancer stages, and may be solid tumors or blood tumors. For example, the tumor may be one or more selected from the group consisting of Barrett's esophagus, colon adenoma and polyp, breast fibroadenoma and cyst, monoclonal gammopathy of undetermined significance (MGUS), monoclonal lymphocytosis, and the like, but is not limited thereto.

The neurological diseases include all neurological diseases capable of exhibiting prophylactic or therapeutic efficacy due to the inhibition of PLK1 activity, and specifically, may be one or more selected from the group consisting of central nervous system disease, neurodegenerative disease, Alzheimer's disease, Parkinson's disease, multiple sclerosis, Huntington's disease, senile dementia, epilepsy, Lou Gehrig, stroke, and nerve damage and axonal degeneration-related disorders following brain or spinal cord injury, but is not limited thereto.

The pharmaceutical composition of the present disclosure may further include one or more active ingredients exhibiting the same or similar medicinal effects in addition to the compound represented by Formula I above, or the pharmaceutically acceptable salt thereof.

An embodiment of the present disclosure is a method of degrading PLK1 by administering a compound represented by Formula I or a pharmaceutically acceptable salt thereof to mammals including humans.

Another embodiment of the present disclosure is a method of degrading PLK1 by administering the compound represented by Formula I or the pharmaceutically acceptable salt thereof to a sample in vitro. The sample may be a cell, a cell culture, a body fluid or tissue of a mammal including a human, but is not limited thereto.

### Advantageous Effects of Invention

The compounds of the present disclosure exhibit an effect of inducing PLK1 degradation. Therefore, the compounds of the present disclosure may be effectively utilized for preventing or treating PLK1-related diseases.

### BRIEF DESCRIPTION OF DRAWINGS

FIG. 1 shows the luciferase assay results by treating Compound 1 to Compound 49 of the present invention.

FIG. 2 shows the luciferase assay results by treating Compound 50 to Compound 90 of the present invention.

FIG. 3 shows the luciferase assay results by treating Compound 91 to Compound 131 of the present invention.

FIG. 4 shows the luciferase assay results by treating Compound 133 to Compound 180 of the present invention.

FIG. 5 shows the luciferase assay results by treating Compound 182 to Compound 225 of the present invention.

### BEST MODE FOR CARRYING OUT THE INVENTION

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

The present disclosure provides synthetic methods for Compound 1 to 225 shown in the table below.

TABLE 1

| Compound | Structure |
|---|---|
| 1 | 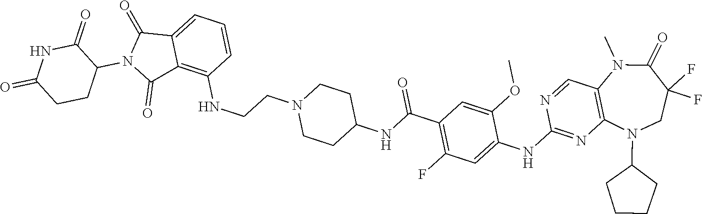 [SMILES: COc1cc(C(=O)NC2CCN(CCNc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 2 | 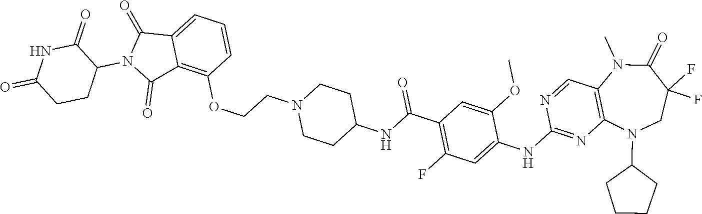 [SMILES: COc1cc(C(=O)NC2CCN(CCOc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 3 | 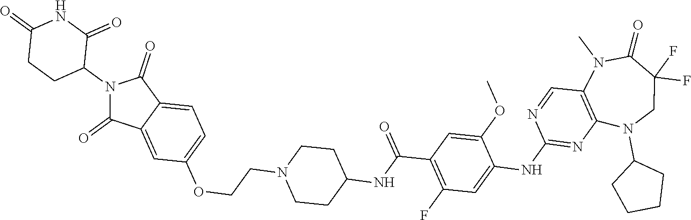 [SMILES: COc1cc(C(=O)NC2CCN(CCOc3ccc4c(c3)C(=O)N(C3CCC(=O)NC3=O)C4=O)CC2)c(F)cc1Nc1ncc2c(n1)N(C1CCCC1)CC(F)(F)C(=O)N2C] |
| 4 | 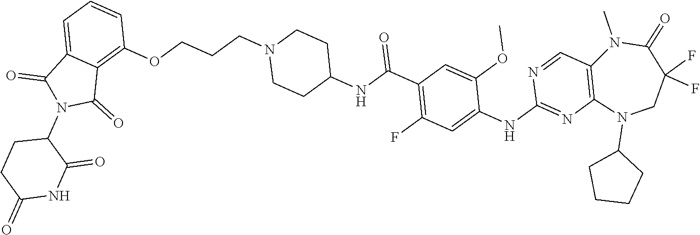 [SMILES: COc1cc(C(=O)NC2CCN(CCCOc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 5 | 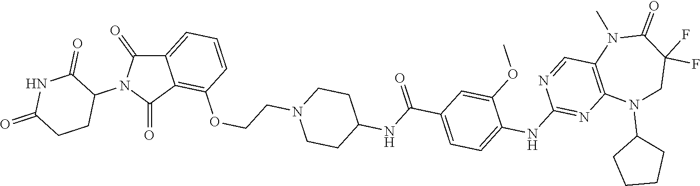 [SMILES: COc1cc(C(=O)NC2CCN(CCOc3cccc4c3C(=O)N(C3CCC(=O)NC3=O)C4=O)CC2)ccc1Nc1ncc2c(n1)N(C1CCCC1)CC(F)(F)C(=O)N2C] |
| 6 | 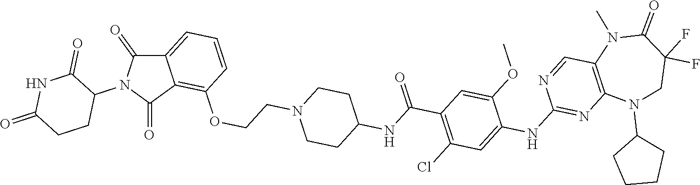 [SMILES: COc1cc(C(=O)NC2CCN(CCOc3cccc4c3C(=O)N(C3CCC(=O)NC3=O)C4=O)CC2)c(Cl)cc1Nc1ncc2c(n1)N(C1CCCC1)CC(F)(F)C(=O)N2C] |
| 7 | 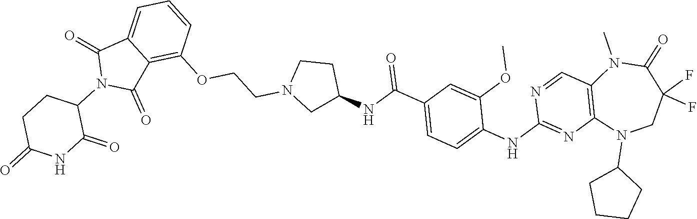 [SMILES: COc1cc(C(=O)N[C@@H]2CCN(CCOc3cccc4c3C(=O)N(C3CCC(=O)NC3=O)C4=O)C2)ccc1Nc1ncc2c(n1)N(C1CCCC1)CC(F)(F)C(=O)N2C] |
| 8 | 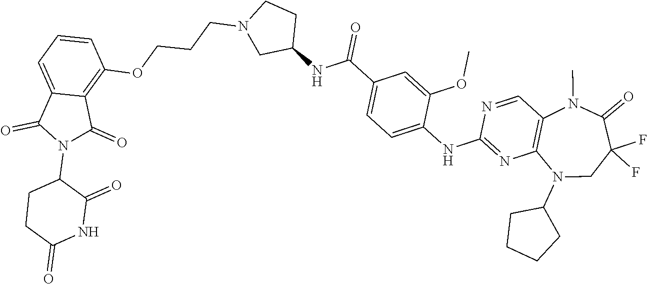 [SMILES: COc1cc(C(=O)N[C@@H]2CCN(CCCOc3cccc4c3C(=O)N(C3CCC(=O)NC3=O)C4=O)C2)ccc1Nc1ncc2c(n1)N(C1CCCC1)CC(F)(F)C(=O)N2C] |
| 9 | 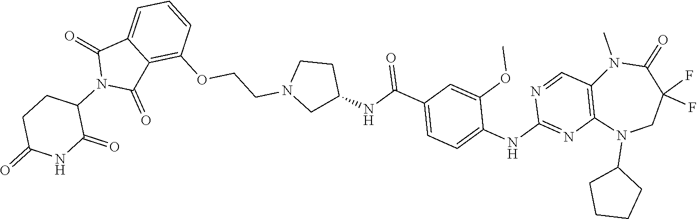 [SMILES: COc1cc(C(=O)N[C@H]2CCN(CCOc3cccc4c3C(=O)N(C3CCC(=O)NC3=O)C4=O)C2)ccc1Nc1ncc2c(n1)N(C1CCCC1)CC(F)(F)C(=O)N2C] |
| 10 | 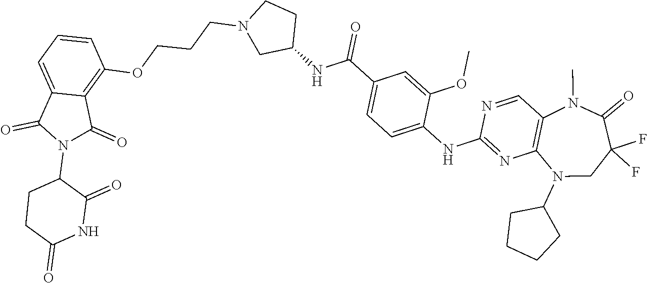 [SMILES: COc1cc(C(=O)N[C@H]2CCN(CCCOc3cccc4c3C(=O)N(C3CCC(=O)NC3=O)C4=O)C2)ccc1Nc1ncc2c(n1)N(C1CCCC1)CC(F)(F)C(=O)N2C] |
| 11 | 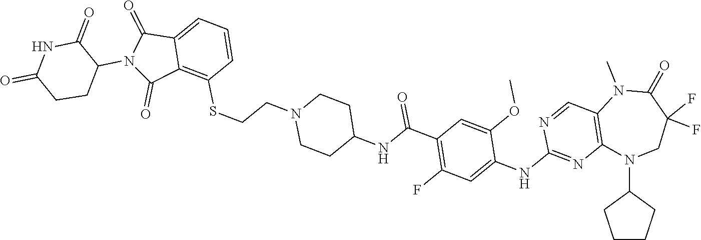 [SMILES: COc1cc(C(=O)NC2CCN(CCSc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 12 | 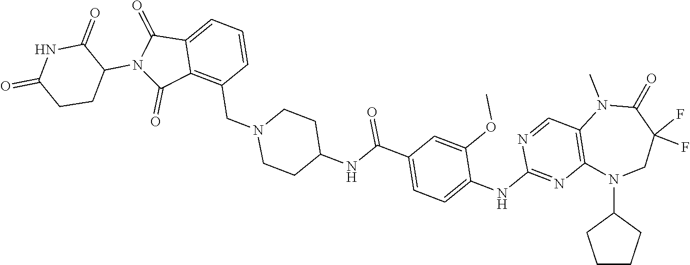 [SMILES: COc1cc(C(=O)NC2CCN(Cc3cccc4c3C(=O)N(C3CCC(=O)NC3=O)C4=O)CC2)ccc1Nc1ncc2c(n1)N(C1CCCC1)CC(F)(F)C(=O)N2C] |
| 13 | 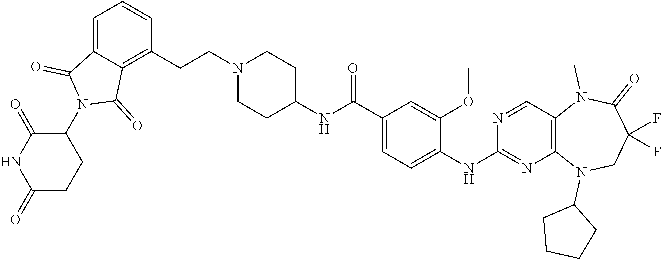 [SMILES: COc1cc(C(=O)NC2CCN(CCc3cccc4c3C(=O)N(C3CCC(=O)NC3=O)C4=O)CC2)ccc1Nc1ncc2c(n1)N(C1CCCC1)CC(F)(F)C(=O)N2C] |
| 14 | 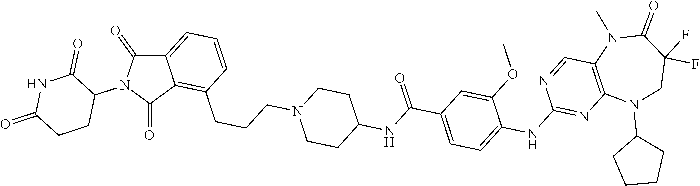 [SMILES: COc1cc(C(=O)NC2CCN(CCCc3cccc4c3C(=O)N(C3CCC(=O)NC3=O)C4=O)CC2)ccc1Nc1ncc2c(n1)N(C1CCCC1)CC(F)(F)C(=O)N2C] |
| 15 | 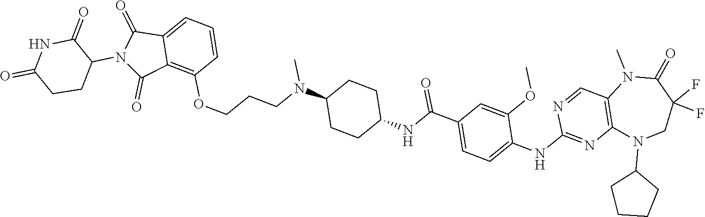 [SMILES: COc1cc(C(=O)N[C@H]2CC[C@H](N(C)CCCOc3cccc4c3C(=O)N(C3CCC(=O)NC3=O)C4=O)CC2)ccc1Nc1ncc2c(n1)N(C1CCCC1)CC(F)(F)C(=O)N2C] |
| 16 | 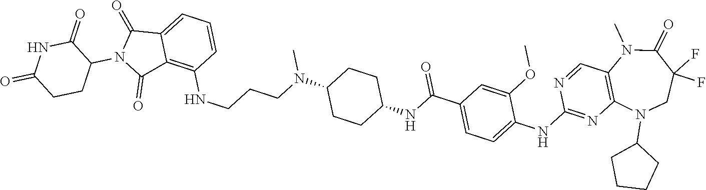 [SMILES: COc1cc(C(=O)N[C@H]2CC[C@@H](N(C)CCCNc3cccc4c3C(=O)N(C3CCC(=O)NC3=O)C4=O)CC2)ccc1Nc1ncc2c(n1)N(C1CCCC1)CC(F)(F)C(=O)N2C] |
| 17 | 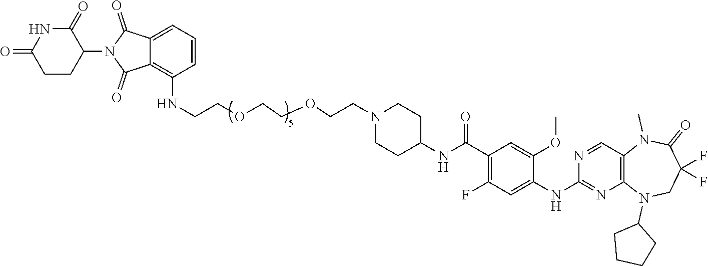 |
| 18 | 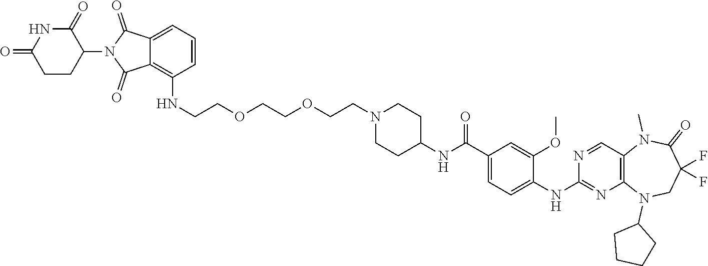 [SMILES: COc1cc(C(=O)NC2CCN(CCOCCOCCNc3cccc4c3C(=O)N(C3CCC(=O)NC3=O)C4=O)CC2)ccc1Nc1ncc2c(n1)N(C1CCCC1)CC(F)(F)C(=O)N2C] |
| 19 | 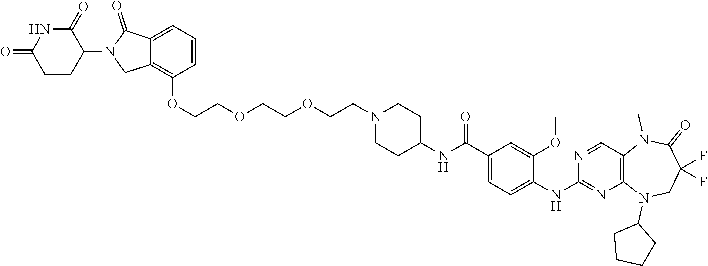 [SMILES: COc1cc(C(=O)NC2CCN(CCOCCOCCOc3cccc4c3CN(C3CCC(=O)NC3=O)C4=O)CC2)ccc1Nc1ncc2c(n1)N(C1CCCC1)CC(F)(F)C(=O)N2C] |
| 20 | 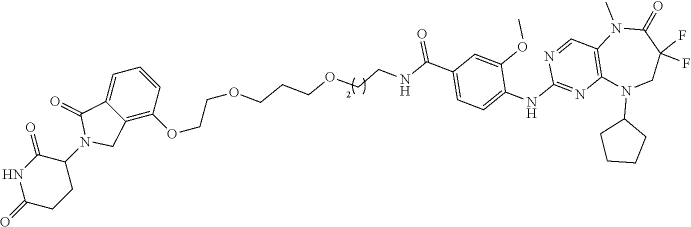 |
| 21 | 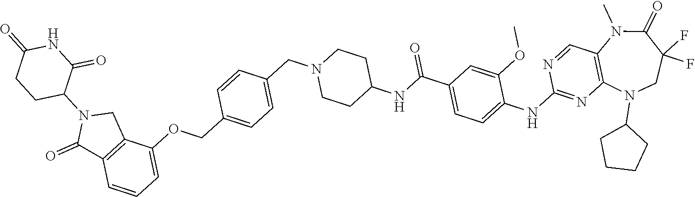 [SMILES: COc1cc(C(=O)NC2CCN(Cc3ccc(COc4cccc5c4CN(C4CCC(=O)NC4=O)C5=O)cc3)CC2)ccc1Nc1ncc2c(n1)N(C1CCCC1)CC(F)(F)C(=O)N2C] |
| 22 | 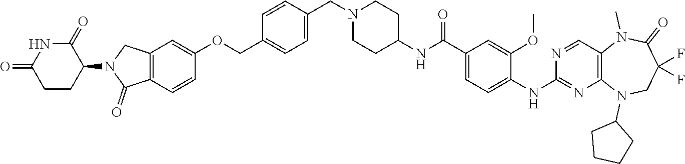 [SMILES: COc1cc(C(=O)NC2CCN(Cc3ccc(COc4ccc5c(c4)CN([C@H]4CCC(=O)NC4=O)C5=O)cc3)CC2)ccc1Nc1ncc2c(n1)N(C1CCCC1)CC(F)(F)C(=O)N2C] |
| 23 | 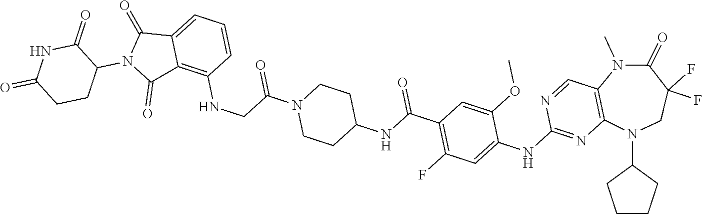 [SMILES: COc1cc(C(=O)NC2CCN(C(=O)CNc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 24 | 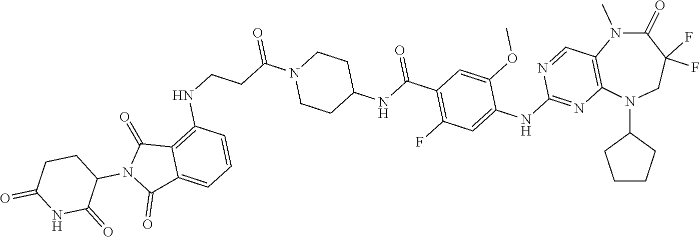 [SMILES: COc1cc(C(=O)NC2CCN(C(=O)CCNc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 25 | 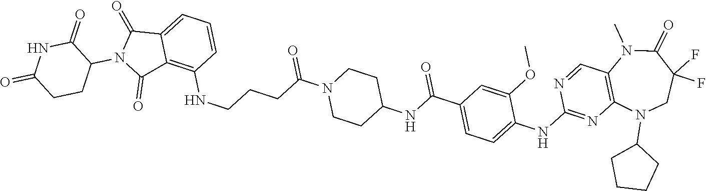 [SMILES: COc1cc(C(=O)NC2CCN(C(=O)CCCNc3cccc4c3C(=O)N(C3CCC(=O)NC3=O)C4=O)CC2)ccc1Nc1ncc2c(n1)N(C1CCCC1)CC(F)(F)C(=O)N2C] |
| 26 | 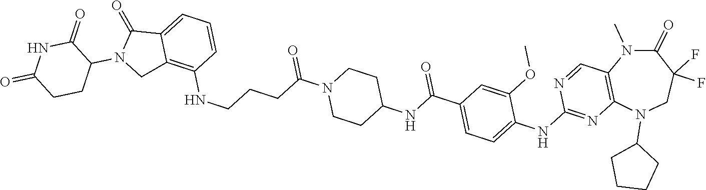 [SMILES: COc1cc(C(=O)NC2CCN(C(=O)CCCNc3cccc4c3CN(C3CCC(=O)NC3=O)C4=O)CC2)ccc1Nc1ncc2c(n1)N(C1CCCC1)CC(F)(F)C(=O)N2C] |
| 27 | 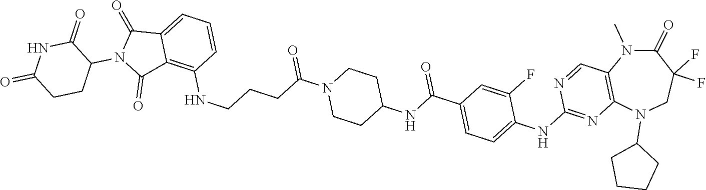 [SMILES: CN1C(=O)C(F)(F)CN(C2CCCC2)c2nc(Nc3ccc(C(=O)NC4CCN(C(=O)CCCNc5cccc6c5C(=O)N(C5CCC(=O)NC5=O)C6=O)CC4)cc3F)ncc21] |
| 28 | 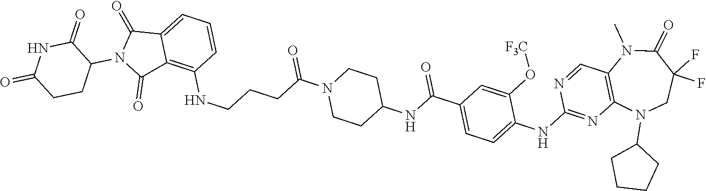 [SMILES: CN1C(=O)C(F)(F)CN(C2CCCC2)c2nc(Nc3ccc(C(=O)NC4CCN(C(=O)CCCNc5cccc6c5C(=O)N(C5CCC(=O)NC5=O)C6=O)CC4)cc3OC(F)(F)F)ncc21] |
| 29 | 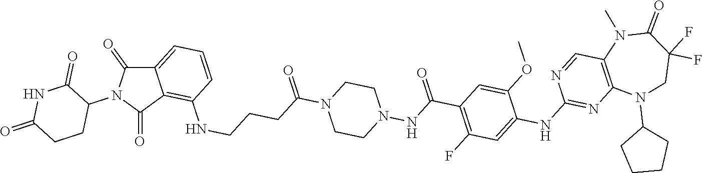 [SMILES: COc1cc(C(=O)NN2CCN(C(=O)CCCNc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 30 | 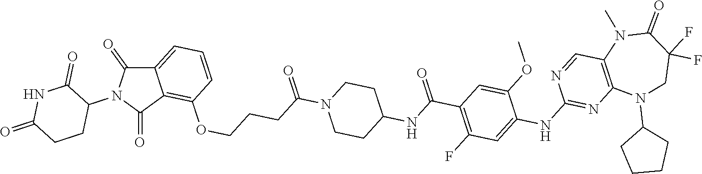 [SMILES: COc1cc(C(=O)NC2CCN(C(=O)CCCOc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 31 | 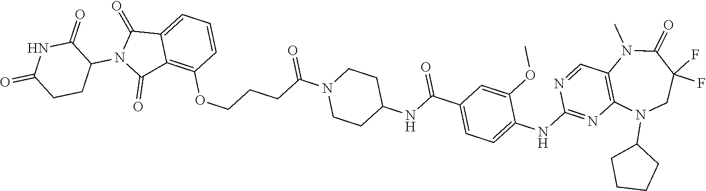 [SMILES: COc1cc(C(=O)NC2CCN(C(=O)CCCOc3cccc4c3C(=O)N(C3CCC(=O)NC3=O)C4=O)CC2)ccc1Nc1ncc2c(n1)N(C1CCCC1)CC(F)(F)C(=O)N2C] |
| 32 | 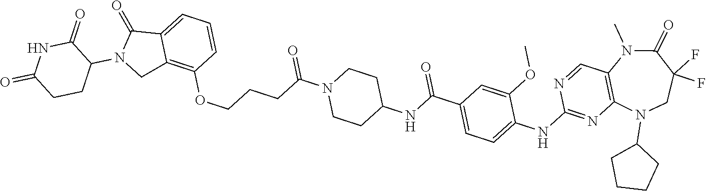 [SMILES: COc1cc(C(=O)NC2CCN(C(=O)CCCOc3cccc4c3CN(C3CCC(=O)NC3=O)C4=O)CC2)ccc1Nc1ncc2c(n1)N(C1CCCC1)CC(F)(F)C(=O)N2C] |
| 33 | 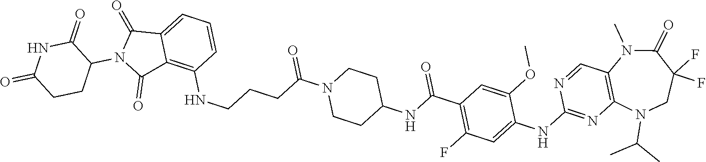 [SMILES: COc1cc(C(=O)NC2CCN(C(=O)CCCNc3cccc4c3C(=O)N(C3CCC(=O)NC3=O)C4=O)CC2)c(F)cc1Nc1ncc2c(n1)N(C(C)C)CC(F)(F)C(=O)N2C] |
| 34 | 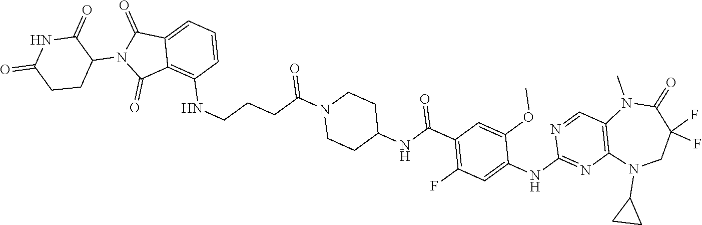 [SMILES: COc1cc(C(=O)NC2CCN(C(=O)CCCNc3cccc4c3C(=O)N(C3CCC(=O)NC3=O)C4=O)CC2)c(F)cc1Nc1ncc2c(n1)N(C1CC1)CC(F)(F)C(=O)N2C] |
| 35 | 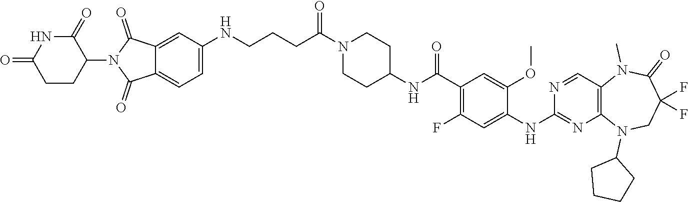 [SMILES: COc1cc(C(=O)NC2CCN(C(=O)CCCNc3ccc4c(c3)C(=O)N(C3CCC(=O)NC3=O)C4=O)CC2)c(F)cc1Nc1ncc2c(n1)N(C1CCCC1)CC(F)(F)C(=O)N2C] |
| 36 | 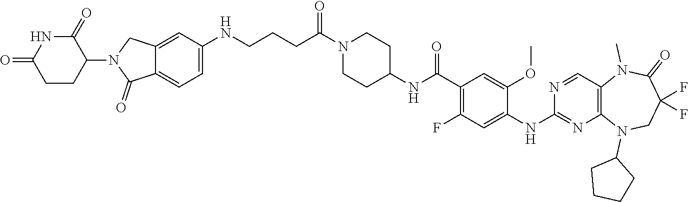 [SMILES: COc1cc(C(=O)NC2CCN(C(=O)CCCNc3ccc4c(c3)CN(C3CCC(=O)NC3=O)C4=O)CC2)c(F)cc1Nc1ncc2c(n1)N(C1CCCC1)CC(F)(F)C(=O)N2C] |
| 37 | 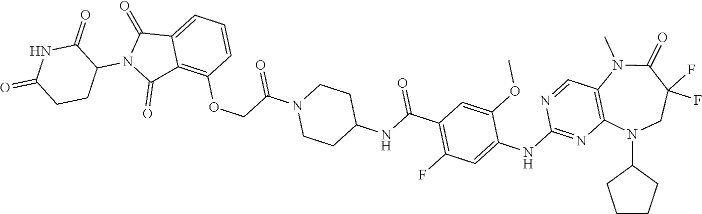 [SMILES: COc1cc(C(=O)NC2CCN(C(=O)COc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 38 | 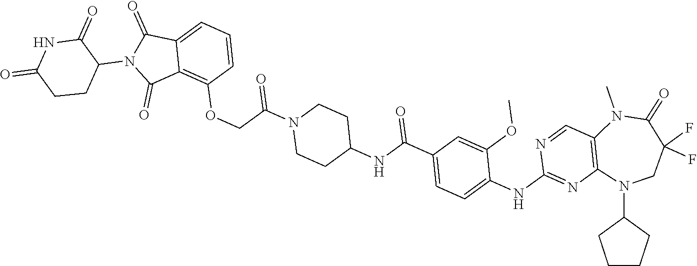 [SMILES: COc1cc(C(=O)NC2CCN(C(=O)COc3cccc4c3C(=O)N(C3CCC(=O)NC3=O)C4=O)CC2)ccc1Nc1ncc2c(n1)N(C1CCCC1)CC(F)(F)C(=O)N2C] |
| 39 | 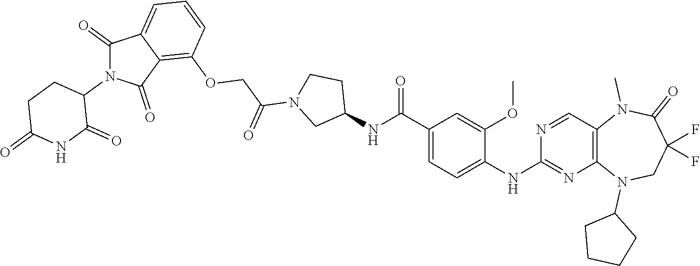 [SMILES: COc1cc(C(=O)N[C@@H]2CCN(C(=O)COc3cccc4c3C(=O)N(C3CCC(=O)NC3=O)C4=O)C2)ccc1Nc1ncc2c(n1)N(C1CCCC1)CC(F)(F)C(=O)N2C] |
| 40 | 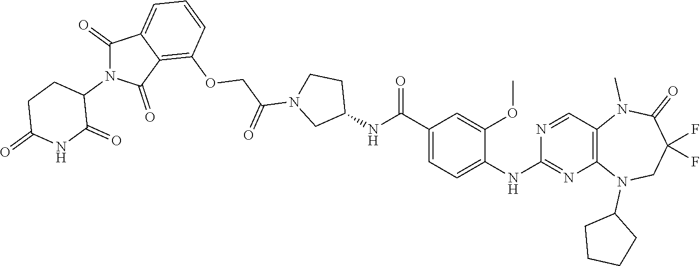 [SMILES: COc1cc(C(=O)N[C@H]2CCN(C(=O)COc3cccc4c3C(=O)N(C3CCC(=O)NC3=O)C4=O)C2)ccc1Nc1ncc2c(n1)N(C1CCCC1)CC(F)(F)C(=O)N2C] |
| 41 | 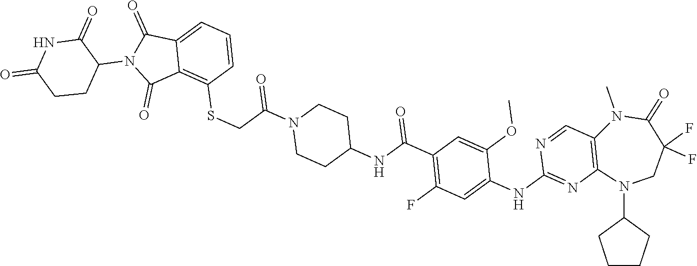 [SMILES: COc1cc(C(=O)NC2CCN(C(=O)CSc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 42 | 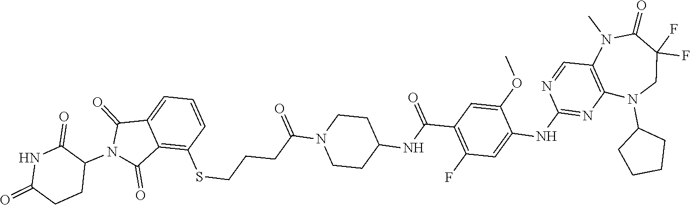 [SMILES: COc1cc(C(=O)NC2CCN(C(=O)CCCSc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 43 | 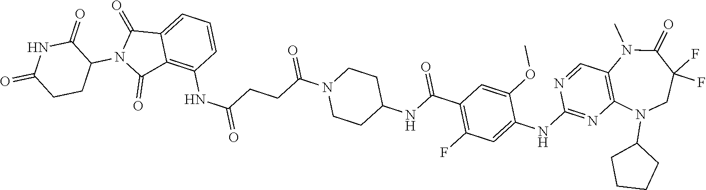 [SMILES: COc1cc(C(=O)NC2CCN(C(=O)CCC(=O)Nc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 44 | 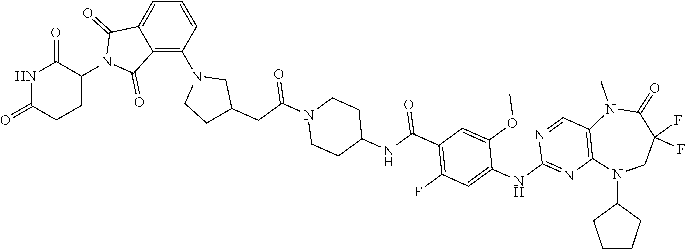 [SMILES: COc1cc(C(=O)NC2CCN(C(=O)CC3CCN(c4cccc5c4C(=O)N(C4CCC(=O)NC4=O)C5=O)C3)CC2)c(F)cc1Nc1ncc2c(n1)N(C1CCCC1)CC(F)(F)C(=O)N2C] |
| 45 | 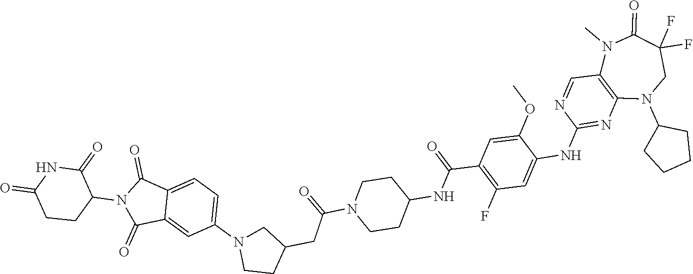 [SMILES: COc1cc(C(=O)NC2CCN(C(=O)CC3CCN(c4ccc5c(c4)C(=O)N(C4CCC(=O)NC4=O)C5=O)C3)CC2)c(F)cc1Nc1ncc2c(n1)N(C1CCCC1)CC(F)(F)C(=O)N2C] |
| 46 | 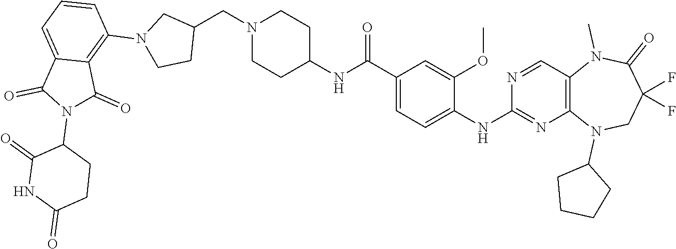 [SMILES: COc1cc(C(=O)NC2CCN(CC3CCN(c4cccc5c4C(=O)N(C4CCC(=O)NC4=O)C5=O)C3)CC2)ccc1Nc1ncc2c(n1)N(C1CCCC1)CC(F)(F)C(=O)N2C] |
| 47 | 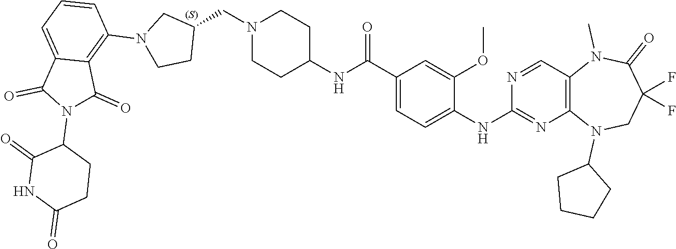 |
| 48 | 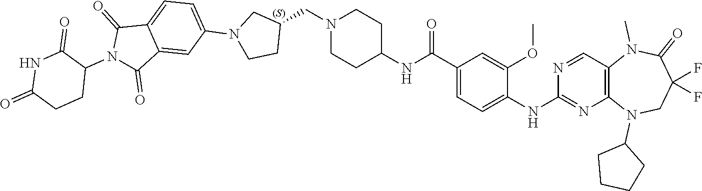 |
| 49 | 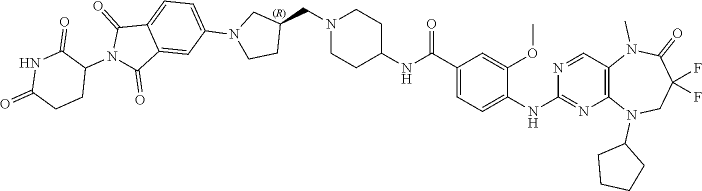 |
| 50 | 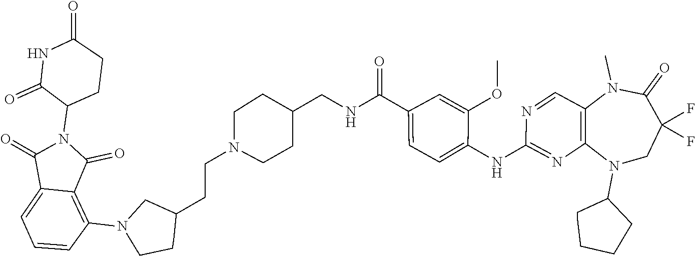 [SMILES: COc1cc(C(=O)NCC2CCN(CCC3CCCN(c4cccc5c4C(=O)N(C4CCC(=O)NC4=O)C5=O)C3)CC2)ccc1Nc1ncc2c(n1)N(C1CCCCC1)CC(F)(F)C(=O)N2C] |
| 51 | 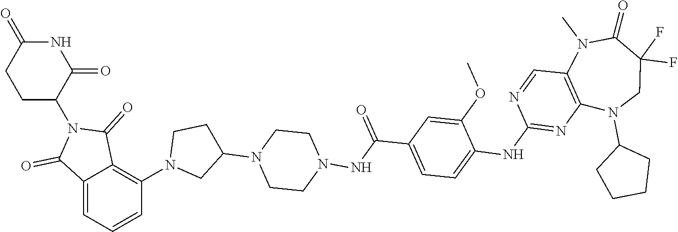 [SMILES: COc1cc(C(=O)NN2CCN(C3CCN(c4cccc5c4C(=O)N(C4CCC(=O)NC4=O)C5=O)C3)CC2)ccc1Nc1ncc2c(n1)N(C1CCCC1)CC(F)(F)C(=O)N2C] |
| 52 | 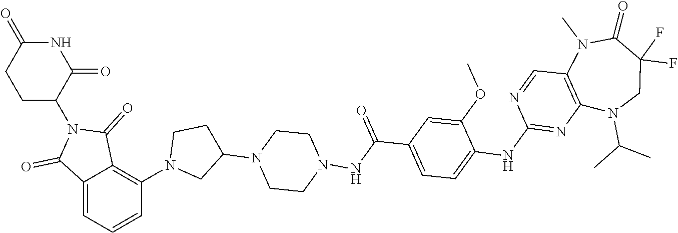 [SMILES: COc1cc(C(=O)NN2CCN(C3CCN(c4cccc5c4C(=O)N(C4CCC(=O)NC4=O)C5=O)C3)CC2)ccc1Nc1ncc2c(n1)N(C(C)C)CC(F)(F)C(=O)N2C] |
| 53 | 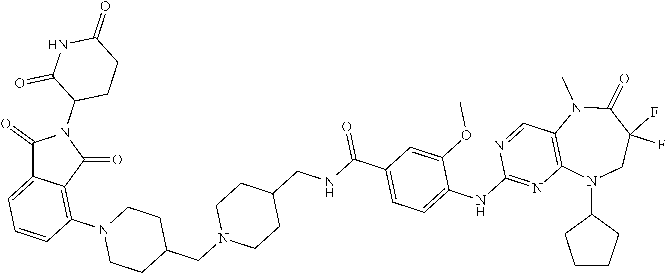 [SMILES: COc1cc(C(=O)NCC2CCN(CC3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 54 | 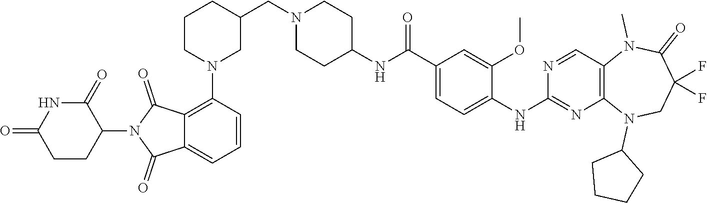 [SMILES: COc1cc(C(=O)NC2CCN(CC3CCCN(c4cccc5c4C(=O)N(C4CCC(=O)NC4=O)C5=O)C3)CC2)ccc1Nc1ncc2c(n1)N(C1CCCC1)CC(F)(F)C(=O)N2C] |
| 55 | 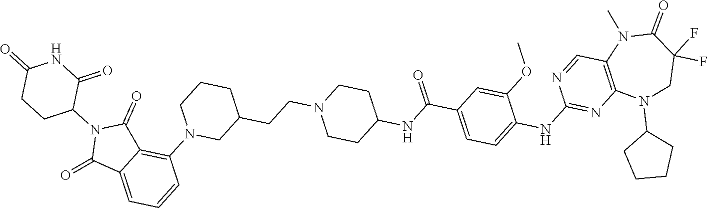 [SMILES: COc1cc(C(=O)NC2CCN(CCC3CCCN(c4cccc5c4C(=O)N(C4CCC(=O)NC4=O)C5=O)C3)CC2)ccc1Nc1ncc2c(n1)N(C1CCCC1)CC(F)(F)C(=O)N2C] |
| 56 | 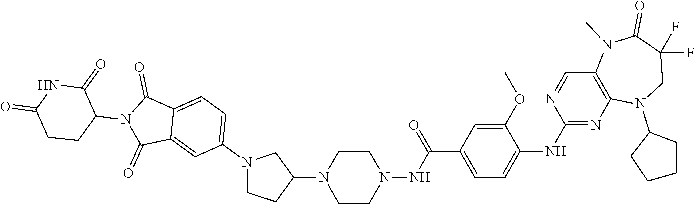 [SMILES: COc1cc(C(=O)NN2CCN(C3CCN(c4ccc5c(c4)C(=O)N(C4CCC(=O)NC4=O)C5=O)C3)CC2)ccc1Nc1ncc2c(n1)N(C1CCCC1)CC(F)(F)C(=O)N2C] |
| 57 | 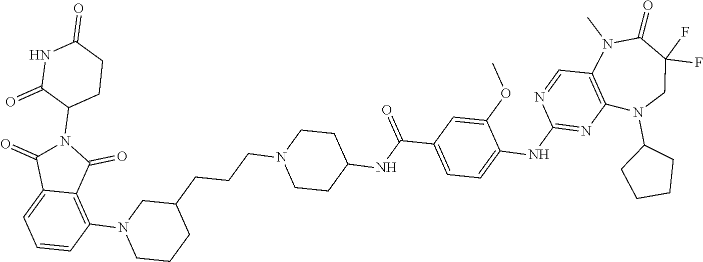 [SMILES: COc1cc(C(=O)NC2CCN(CCCC3CCCN(c4cccc5c4C(=O)N(C4CCC(=O)NC4=O)C5=O)C3)CC2)ccc1Nc1ncc2c(n1)N(C1CCCC1)CC(F)(F)C(=O)N2C] |
| 58 | 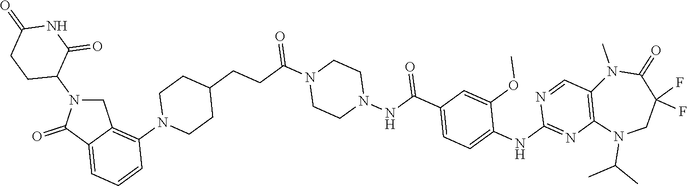 [SMILES: COc1cc(C(=O)NN2CCN(C(=O)CCC3CCN(c4cccc5c4CN(C4CCC(=O)NC4=O)C5=O)CC3)CC2)ccc1Nc1ncc2c(n1)N(C(C)C)CC(F)(F)C(=O)N2C] |
| 59 | 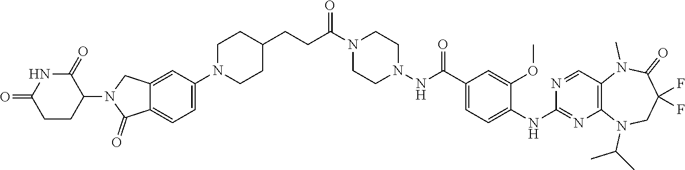 [SMILES: COc1cc(C(=O)NN2CCN(C(=O)CCC3CCN(c4ccc5c(c4)CN(C4CCC(=O)NC4=O)C5=O)CC3)CC2)ccc1Nc1ncc2c(n1)N(C(C)C)CC(F)(F)C(=O)N2C] |
| 60 | 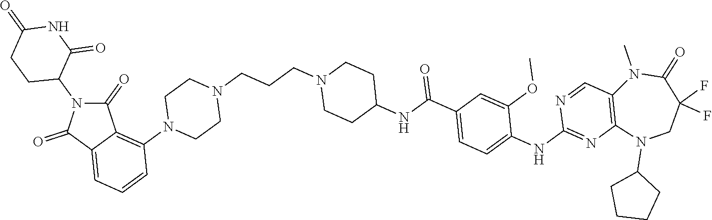 [SMILES: COc1cc(C(=O)NC2CCN(CCCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 61 | 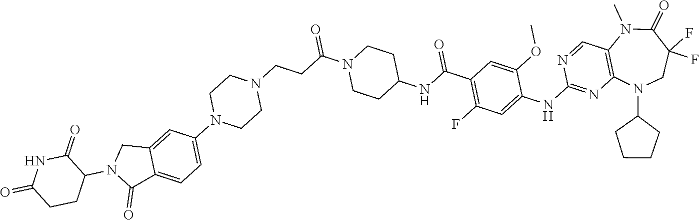 [SMILES: COc1cc(C(=O)NC2CCN(C(=O)CCN3CCN(c4ccc5c(c4)CN(C4CCC(=O)NC4=O)C5=O)CC3)CC2)c(F)cc1Nc1ncc2c(n1)N(C1CCCC1)CC(F)(F)C(=O)N2C] |
| 62 | 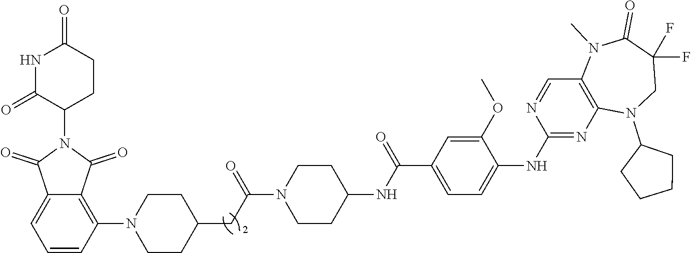 |
| 63 | 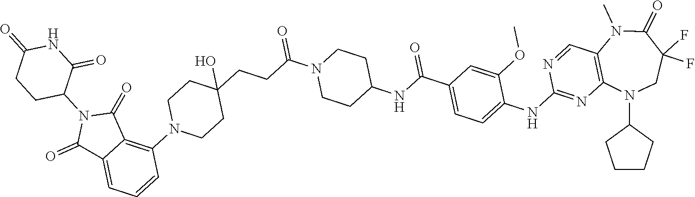 [SMILES: COc1cc(C(=O)NC2CCN(C(=O)CCC3(O)CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 64 | 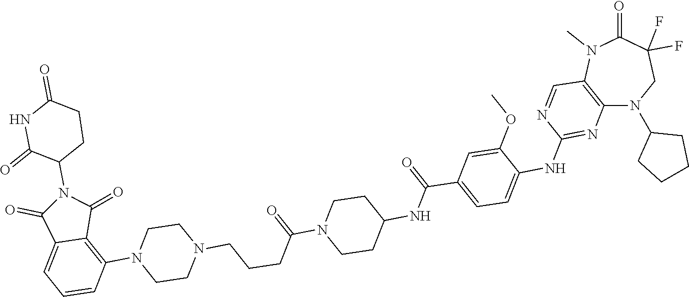 [SMILES: COc1cc(C(=O)NC2CCN(C(=O)CCCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 65 | 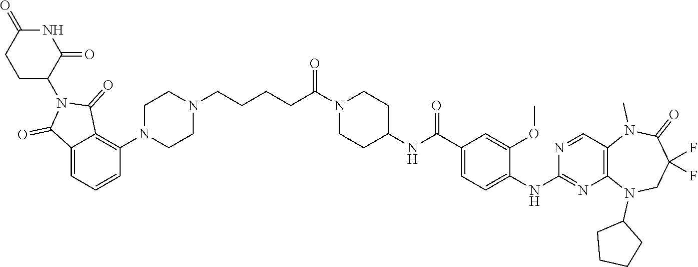 [SMILES: COc1cc(C(=O)NC2CCN(C(=O)CCCCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 66 | 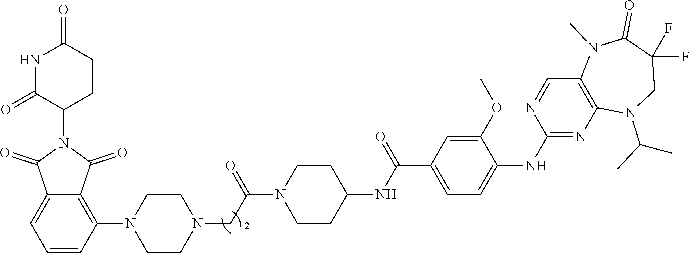 |
| 67 | 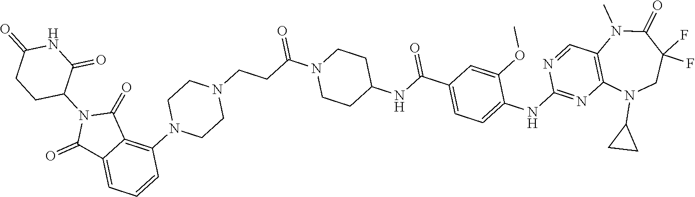 [SMILES: COc1cc(C(=O)NC2CCN(C(=O)CCN3CCN(c4cccc5c4C(=O)N(C4CCC(=O)NC4=O)C5=O)CC3)CC2)ccc1Nc1ncc2c(n1)N(C1CC1)CC(F)(F)C(=O)N2C] |
| 68 | 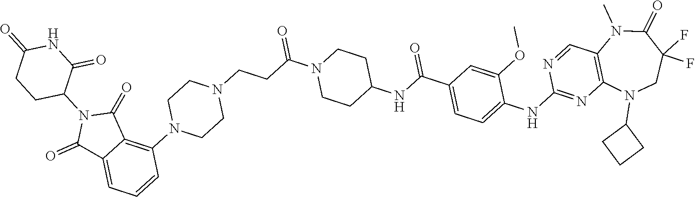 [SMILES: COc1cc(C(=O)NC2CCN(C(=O)CCN3CCN(c4cccc5c4C(=O)N(C4CCC(=O)NC4=O)C5=O)CC3)CC2)ccc1Nc1ncc2c(n1)N(C1CCC1)CC(F)(F)C(=O)N2C] |
| 69 | 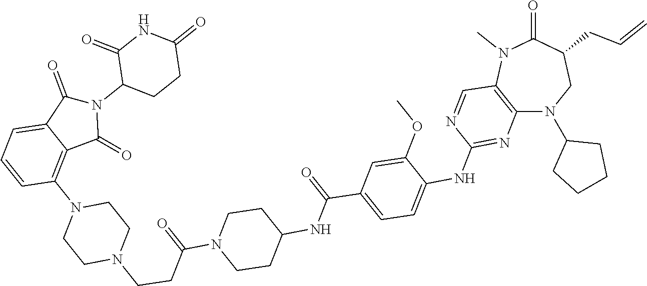 [SMILES: C=CC[C@@H]1CN(C2CCCC2)c2nc(Nc3ccc(C(=O)NC4CCN(C(=O)CCN5CCN(c6cccc7c6C(=O)N(C6CCC(=O)NC6=O)C7=O)CC5)CC4)cc3OC)ncc2N(C)C1=O] |
| 70 | 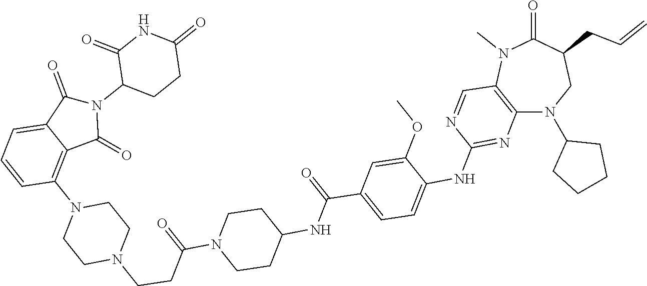 [SMILES: C=CC[C@H]1CN(C2CCCC2)c2nc(Nc3ccc(C(=O)NC4CCN(C(=O)CCN5CCN(c6cccc7c6C(=O)N(C6CCC(=O)NC6=O)C7=O)CC5)CC4)cc3OC)ncc2N(C)C1=O] |
| 71 | 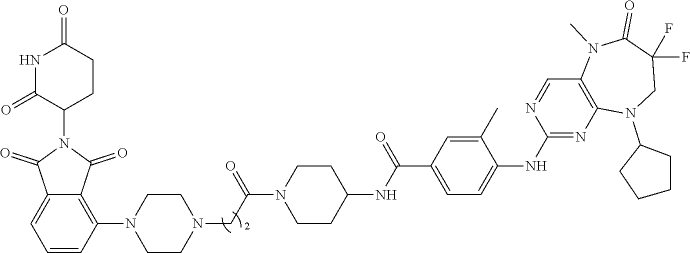 |
| 72 | 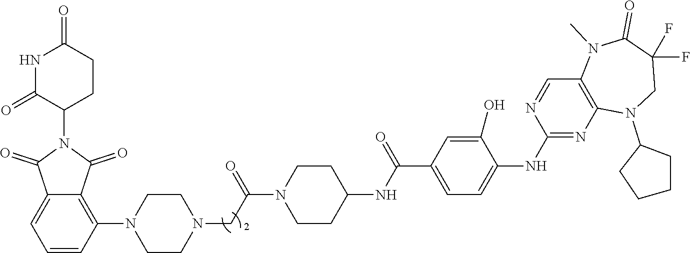 |
| 73 | 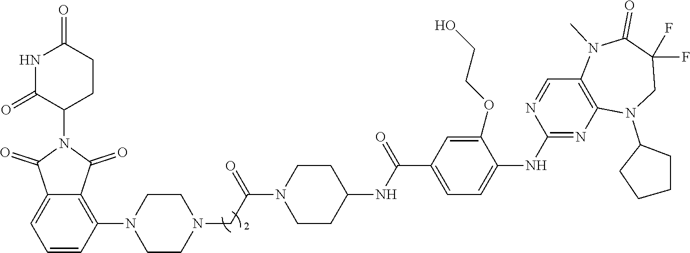 |
| 74 | 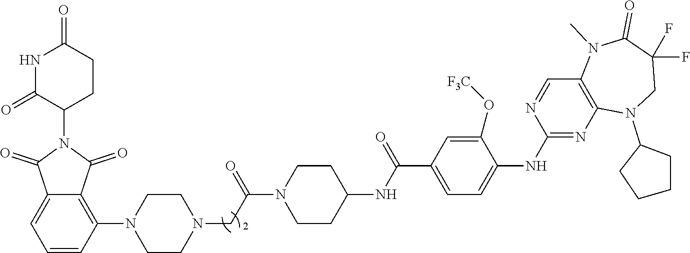 |
| 75 | 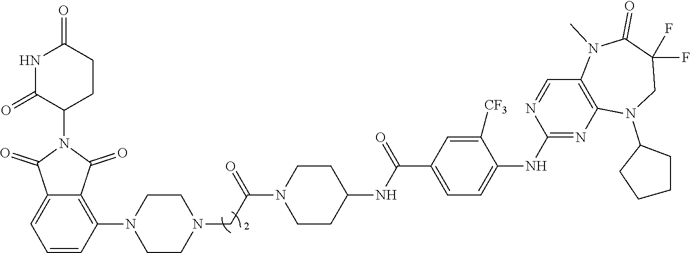 |
| 76 | 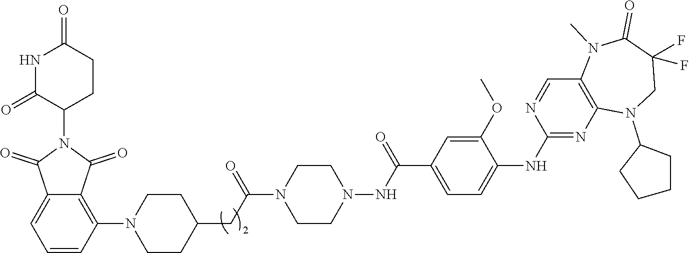 |
| 77 | 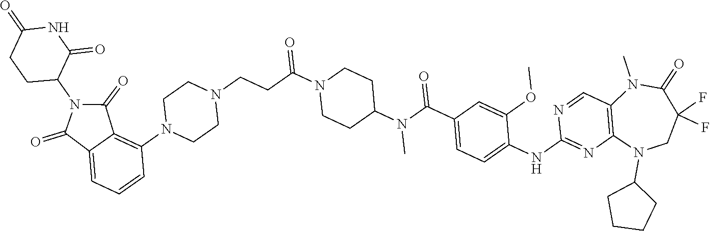 [SMILES: COc1cc(C(=O)N(C)C2CCN(C(=O)CCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 78 | 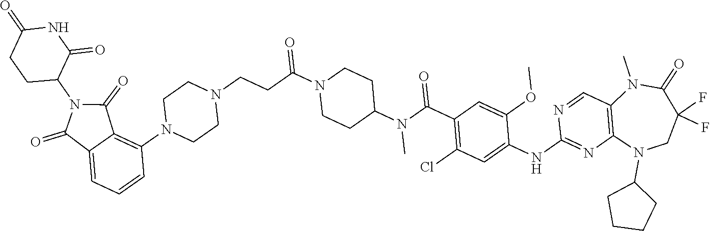 [SMILES: COc1cc(C(=O)N(C)C2CCN(C(=O)CCN3CCN(c4cccc5c4C(=O)N(C4CCC(=O)NC4=O)C5=O)CC3)CC2)c(Cl)cc1Nc1ncc2c(n1)N(C1CCCC1)CC(F)(F)C(=O)N2C] |
| 79 | 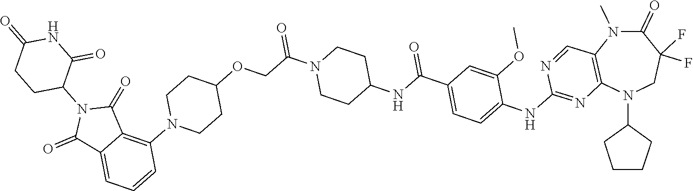 [SMILES: COc1cc(C(=O)NC2CCN(C(=O)COC3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 80 | 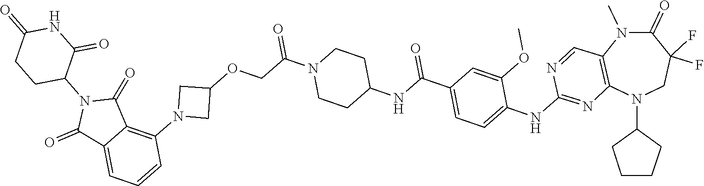 [SMILES: COc1cc(C(=O)NC2CCN(C(=O)COC3CN(c4cccc5c4C(=O)N(C4CCC(=O)NC4=O)C5=O)C3)CC2)ccc1Nc1ncc2c(n1)N(C1CCCC1)CC(F)(F)C(=O)N2C] |
| 81 | 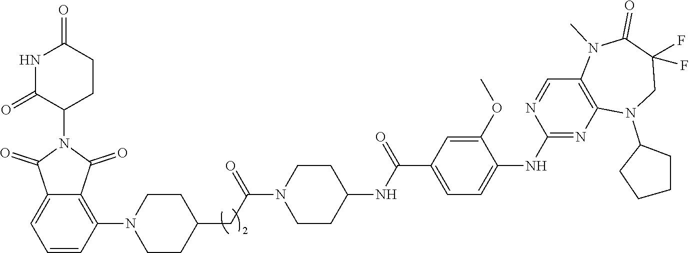 |
| 82 | 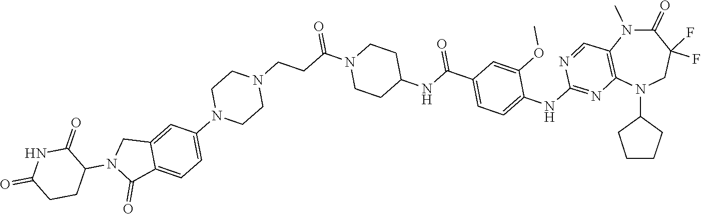 [SMILES: COc1cc(C(=O)NC2CCN(C(=O)CCN3CCN(c4ccc5c(c4)CN(C4CCC(=O)NC4=O)C5=O)CC3)CC2)ccc1Nc1ncc2c(n1)N(C1CCCC1)CC(F)(F)C(=O)N2C] |
| 83 | 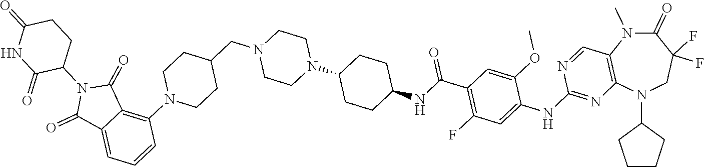 [SMILES: COc1cc(C(=O)N[C@H]2CC[C@H](N3CCN(CC4CCN(c5cccc6c5C(=O)N(C5CCC(=O)NC5=O)C6=O)CC4)CC3)CC2)c(F)cc1Nc1ncc2c(n1)N(C1CCCC1)CC(F)(F)C(=O)N2C] |
| 84 | 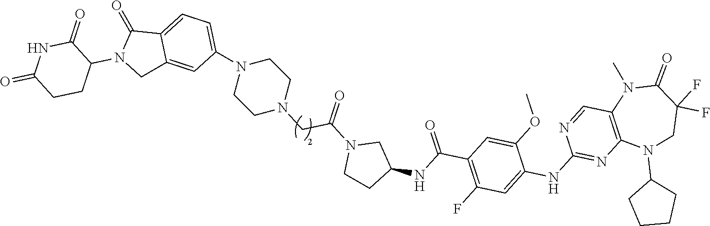 |
| 85 | 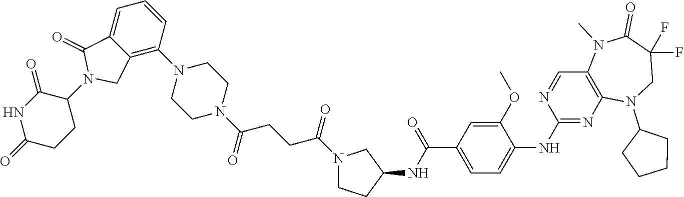 [SMILES: COc1cc(C(=O)N[C@H]2CCN(C(=O)CCC(=O)N3CCN(c4cccc5c4CN(C4CCC(=O)NC4=O)C5=O)CC3)C2)ccc1Nc1ncc2c(n1)N(C1CCCC1)CC(F)(F)C(=O)N2C] |
| 86 | 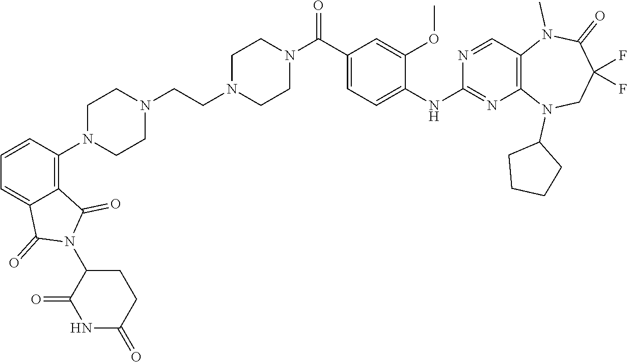 [SMILES: COc1cc(C(=O)N2CCN(CCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 87 | 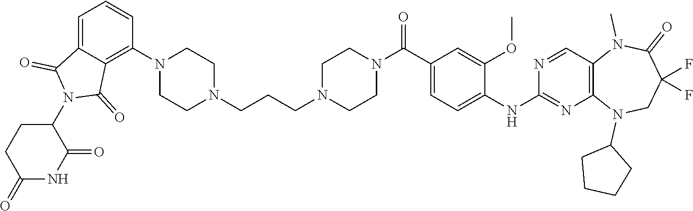 [SMILES: COc1cc(C(=O)N2CCN(CCCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 88 | 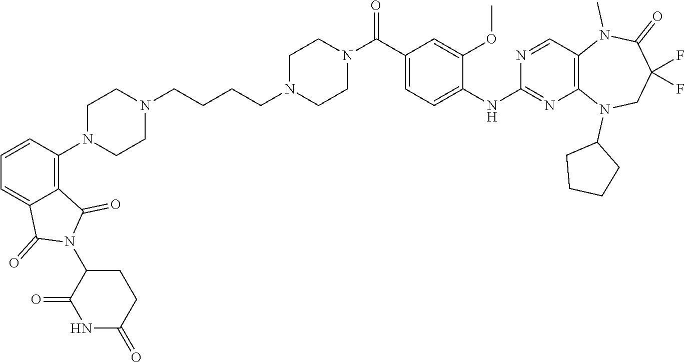 [SMILES: COc1cc(C(=O)N2CCN(CCCCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 89 | 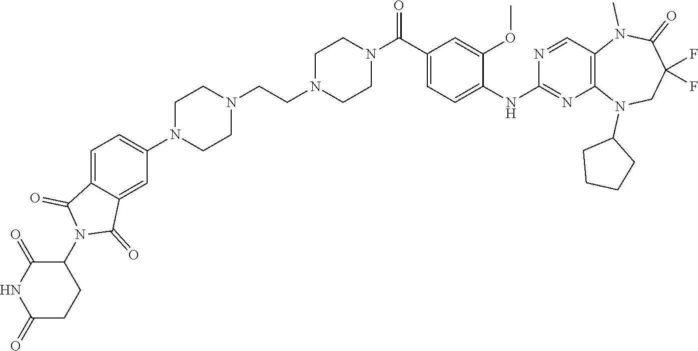 [SMILES: COc1cc(C(=O)N2CCN(CCN3CCN(c4ccc5c(c4)C(=O)N(C4CCC(=O)NC4=O)C5=O)CC3)CC2)ccc1Nc1ncc2c(n1)N(C1CCCC1)CC(F)(F)C(=O)N2C] |
| 90 | 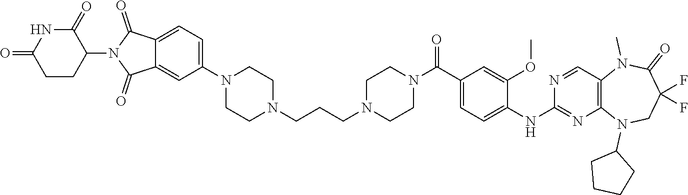 [SMILES: COc1cc(C(=O)N2CCN(CCCN3CCN(c4ccc5c(c4)C(=O)N(C4CCC(=O)NC4=O)C5=O)CC3)CC2)ccc1Nc1ncc2c(n1)N(C1CCCC1)CC(F)(F)C(=O)N2C] |
| 91 | 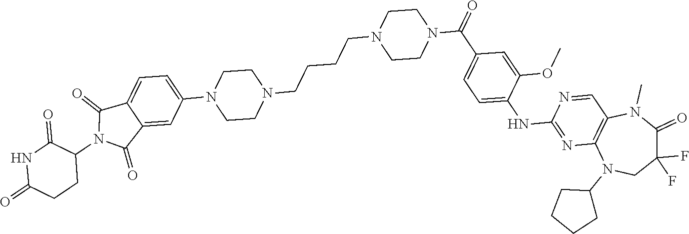 [SMILES: COc1cc(C(=O)N2CCN(CCCCN3CCN(c4ccc5c(c4)C(=O)N(C4CCC(=O)NC4=O)C5=O)CC3)CC2)ccc1Nc1ncc2c(n1)N(C1CCCC1)CC(F)(F)C(=O)N2C] |
| 92 | 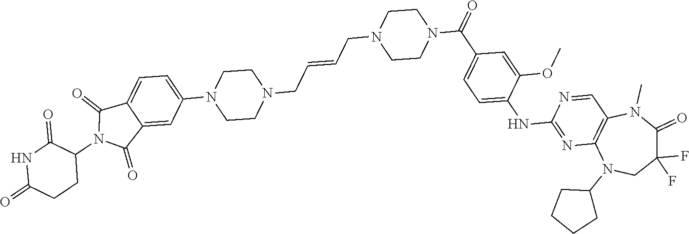 [SMILES: COc1cc(C(=O)N2CCN(C/C=C/CN3CCN(c4ccc5c(c4)C(=O)N(C4CCC(=O)NC4=O)C5=O)CC3)CC2)ccc1Nc1ncc2c(n1)N(C1CCCC1)CC(F)(F)C(=O)N2C] |
| 93 | 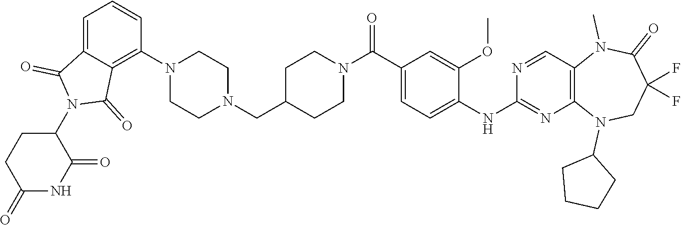 [SMILES: COc1cc(C(=O)N2CCC(CN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 94 | 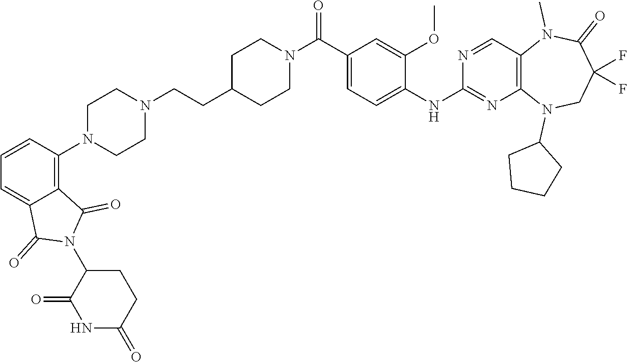 [SMILES: COc1cc(C(=O)N2CCC(CCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 95 | 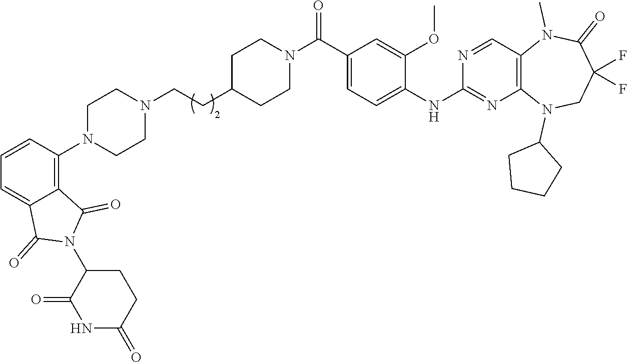 |
| 96 | 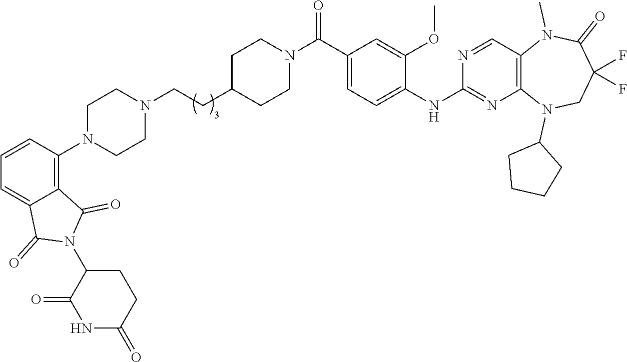 |
| 97 | 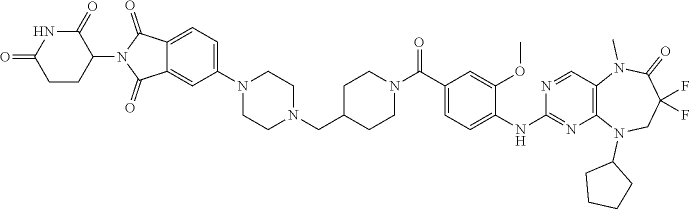 [SMILES: COc1cc(C(=O)N2CCC(CN3CCN(c4ccc5c(c4)C(=O)N(C4CCC(=O)NC4=O)C5=O)CC3)CC2)ccc1Nc1ncc2c(n1)N(C1CCCC1)CC(F)(F)C(=O)N2C] |
| 98 | 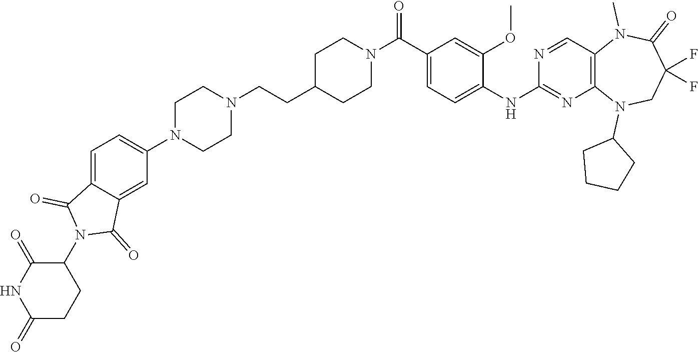 [SMILES: COc1cc(C(=O)N2CCC(CCN3CCN(c4ccc5c(c4)C(=O)N(C4CCC(=O)NC4=O)C5=O)CC3)CC2)ccc1Nc1ncc2c(n1)N(C1CCCC1)CC(F)(F)C(=O)N2C] |
| 99 | 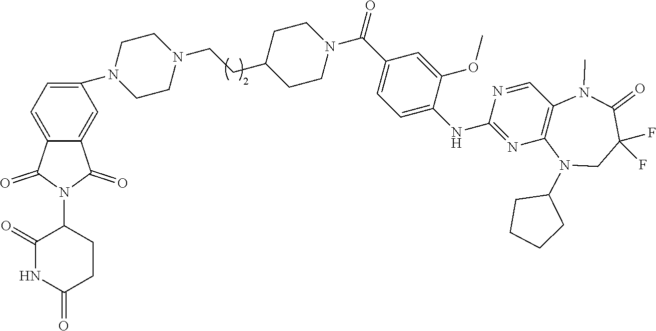 |
| 100 | 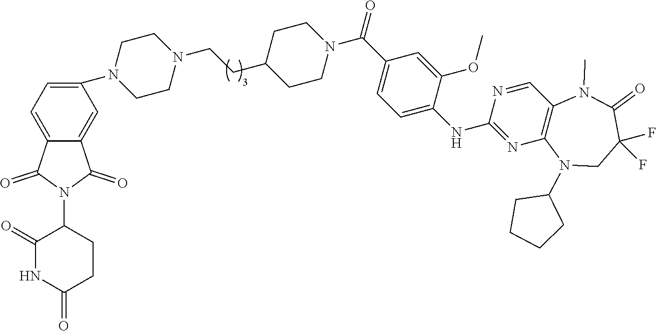 |
| 101 | 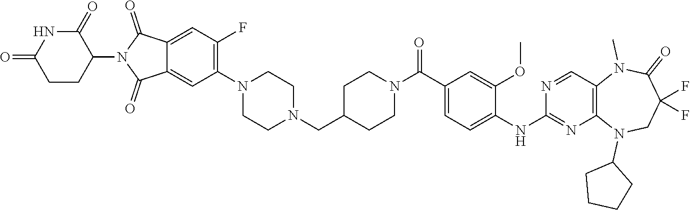 [SMILES: COc1cc(C(=O)N2CCC(CN3CCN(c4cc5c(cc4F)C(=O)N(C4CCC(=O)NC4=O)C5=O)CC3)CC2)ccc1Nc1ncc2c(n1)N(C1CCCC1)CC(F)(F)C(=O)N2C] |
| 102 | 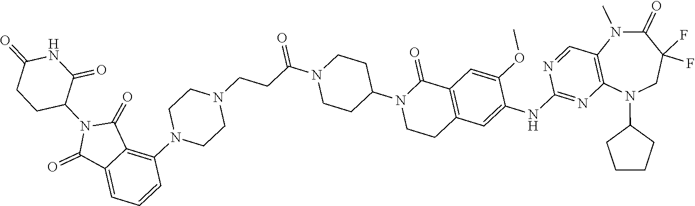 [SMILES: COc1cc2c(cc1Nc1ncc3c(n1)N(C1CCCC1)CC(F)(F)C(=O)N3C)CCN(C1CCN(C(=O)CCN3CCN(c4cccc5c4C(=O)N(C4CCC(=O)NC4=O)C5=O)CC3)CC1)C2=O] |
| 103 | 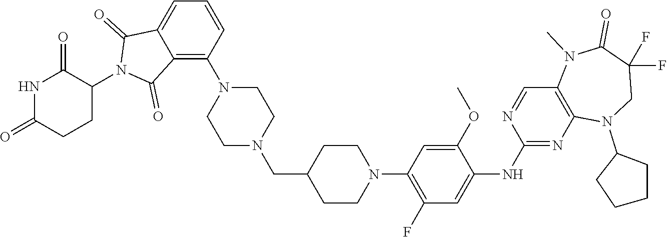 [SMILES: COc1cc(N2CCC(CN3CCN(c4cccc5c4C(=O)N(C4CCC(=O)NC4=O)C5=O)CC3)CC2)c(F)cc1Nc1ncc2c(n1)N(C1CCCC1)CC(F)(F)C(=O)N2C] |
| 104 | 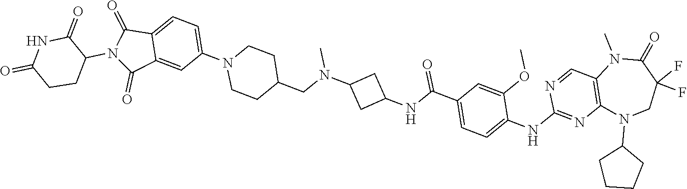 [SMILES: COc1cc(C(=O)NC2CC(N(C)CC3CCN(c4ccc5c(c4)C(=O)N(C4CCC(=O)NC4=O)C5=O)CC3)C2)ccc1Nc1ncc2c(n1)N(C1CCCC1)CC(F)(F)C(=O)N2C] |
| 105 | 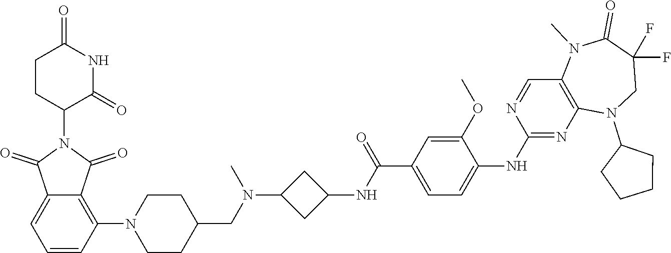 [SMILES: COc1cc(C(=O)NC2CC(N(C)CC3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 106 | 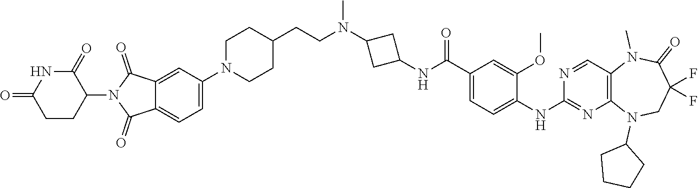 [SMILES: COc1cc(C(=O)NC2CC(N(C)CCC3CCN(c4ccc5c(c4)C(=O)N(C4CCC(=O)NC4=O)C5=O)CC3)C2)ccc1Nc1ncc2c(n1)N(C1CCCC1)CC(F)(F)C(=O)N2C] |
| 107 | 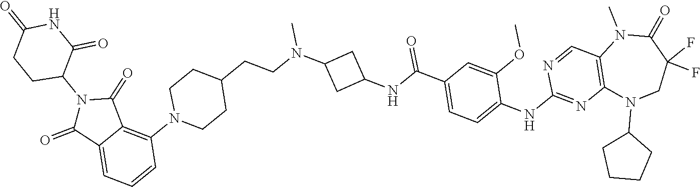 [SMILES: COc1cc(C(=O)NC2CC(N(C)CCC3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 108 | 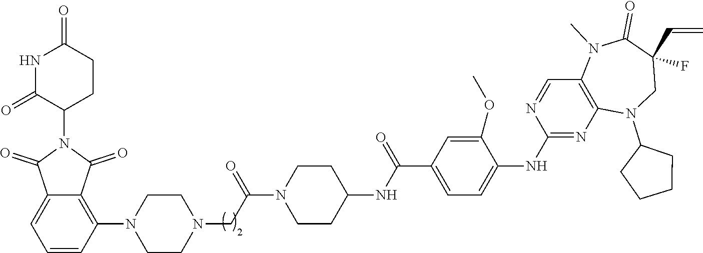 |
| 109 | 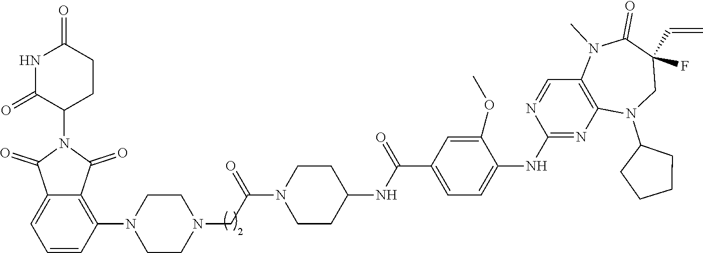 |
| 110 | 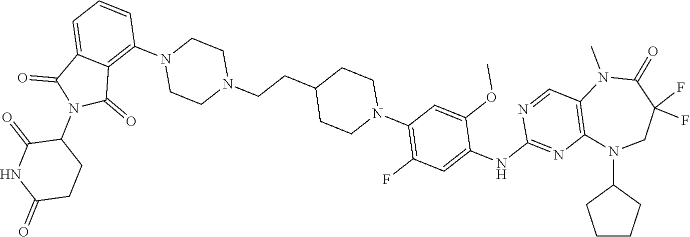 [SMILES: COc1cc(N2CCC(CCN3CCN(c4cccc5c4C(=O)N(C4CCC(=O)NC4=O)C5=O)CC3)CC2)c(F)cc1Nc1ncc2c(n1)N(C1CCCC1)CC(F)(F)C(=O)N2C] |
| 111 | 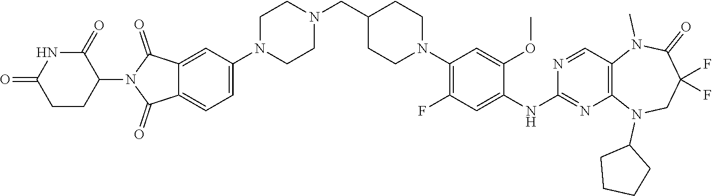 [SMILES: COc1cc(N2CCC(CN3CCN(c4ccc5c(c4)C(=O)N(C4CCC(=O)NC4=O)C5=O)CC3)CC2)c(F)cc1Nc1ncc2c(n1)N(C1CCCC1)CC(F)(F)C(=O)N2C] |
| 112 | 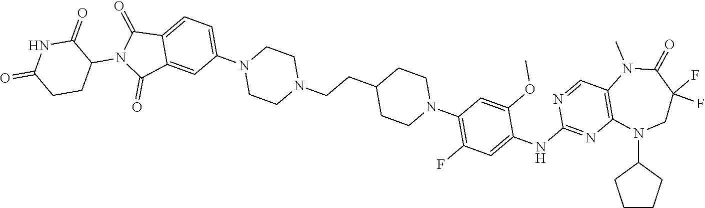 [SMILES: COc1cc(N2CCC(CCN3CCN(c4ccc5c(c4)C(=O)N(C4CCC(=O)NC4=O)C5=O)CC3)CC2)c(F)cc1Nc1ncc2c(n1)N(C1CCCC1)CC(F)(F)C(=O)N2C] |
| 113 | 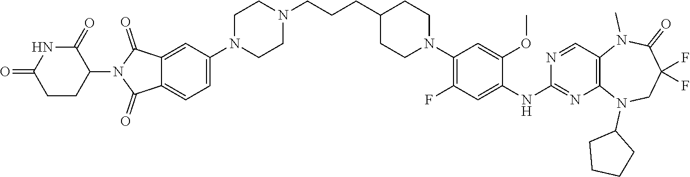 [SMILES: COc1cc(N2CCC(CCCN3CCN(c4ccc5c(c4)C(=O)N(C4CCC(=O)NC4=O)C5=O)CC3)CC2)c(F)cc1Nc1ncc2c(n1)N(C1CCCC1)CC(F)(F)C(=O)N2C] |
| 114 | 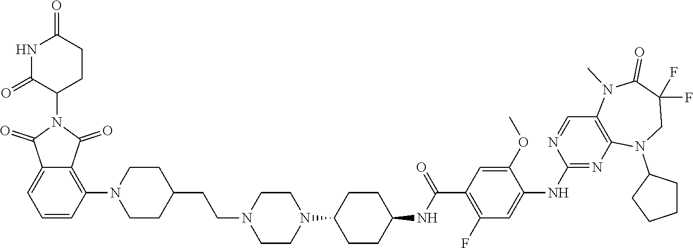 [SMILES: COc1cc(C(=O)N[C@H]2CC[C@H](N3CCN(CCC4CCN(c5cccc6c5C(=O)N(C5CCC(=O)NC5=O)C6=O)CC4)CC3)CC2)c(F)cc1Nc1ncc2c(n1)N(C1CCCC1)CC(F)(F)C(=O)N2C] |
| 115 | 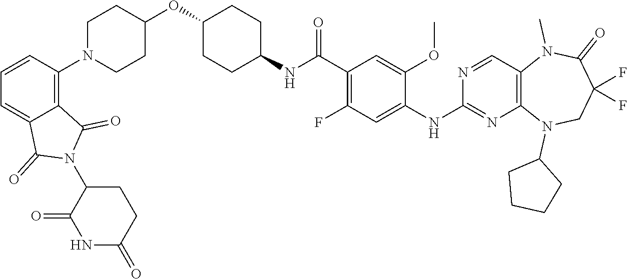 [SMILES: COc1cc(C(=O)N[C@H]2CC[C@H](OC3CCN(c4cccc5c4C(=O)N(C4CCC(=O)NC4=O)C5=O)CC3)CC2)c(F)cc1Nc1ncc2c(n1)N(C1CCCC1)CC(F)(F)C(=O)N2C] |
| 116 | 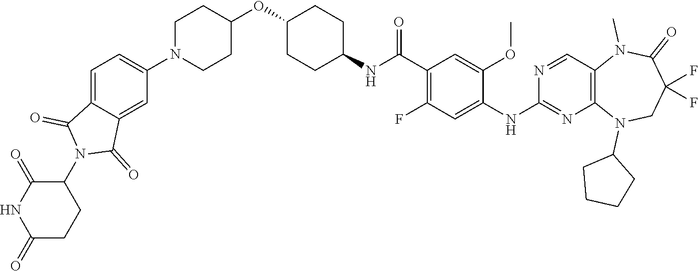 [SMILES: COc1cc(C(=O)N[C@H]2CC[C@H](OC3CCN(c4ccc5c(c4)C(=O)N(C4CCC(=O)NC4=O)C5=O)CC3)CC2)c(F)cc1Nc1ncc2c(n1)N(C1CCCC1)CC(F)(F)C(=O)N2C] |
| 117 | 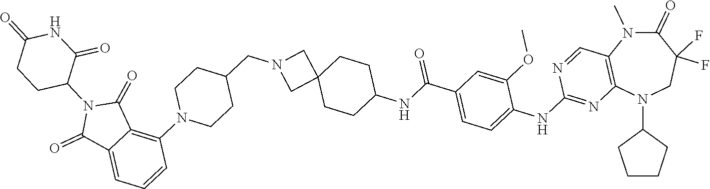 [SMILES: COc1cc(C(=O)NC2CCC3(CC2)CN(CC2CCN(c4cccc5c4C(=O)N(C4CCC(=O)NC4=O)C5=O)CC2)C3)ccc1Nc1ncc2c(n1)N(C1CCCC1)CC(F)(F)C(=O)N2C] |
| 118 | 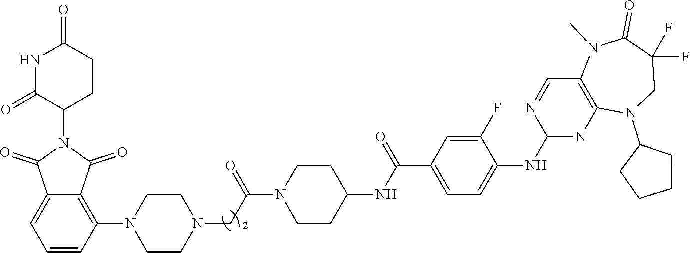 |
| 119 | 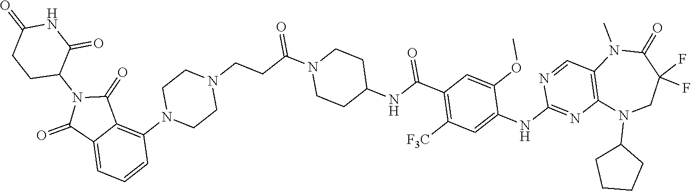 [SMILES: COc1cc(C(=O)NC2CCN(C(=O)CCN3CCN(c4cccc5c4C(=O)N(C4CCC(=O)NC4=O)C5=O)CC3)CC2)c(C(F)(F)F)cc1Nc1ncc2c(n1)N(C1CCCC1)CC(F)(F)C(=O)N2C] |
| 120 | 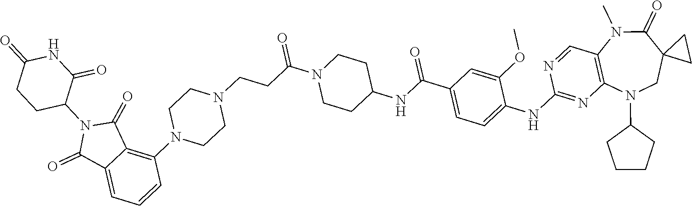 [SMILES: COc1cc(C(=O)NC2CCN(C(=O)CCN3CCN(c4cccc5c4C(=O)N(C4CCC(=O)NC4=O)C5=O)CC3)CC2)ccc1Nc1ncc2c(n1)N(C1CCCC1)CC1(CC1)C(=O)N2C] |
| 121 | 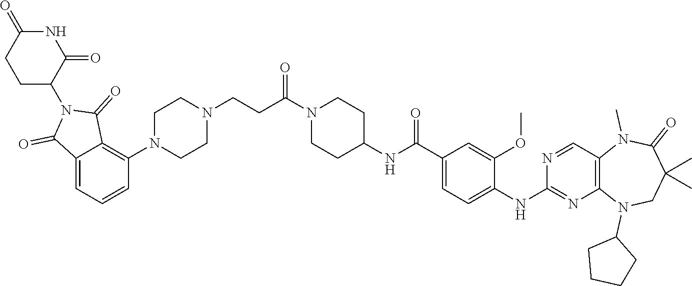 [SMILES: COc1cc(C(=O)NC2CCN(C(=O)CCN3CCN(c4cccc5c4C(=O)N(C4CCC(=O)NC4=O)C5=O)CC3)CC2)ccc1Nc1ncc2c(n1)N(C1CCCC1)CC(C)(C)C(=O)N2C] |
| 122 | 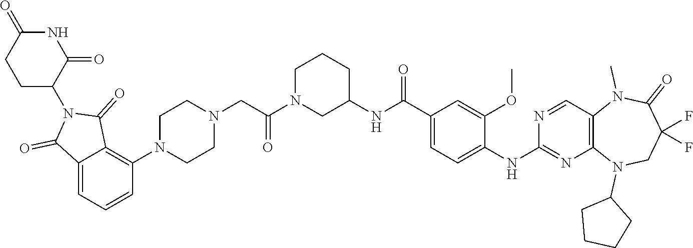 [SMILES: COc1cc(C(=O)NC2CCCN(C(=O)CN3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 123 | 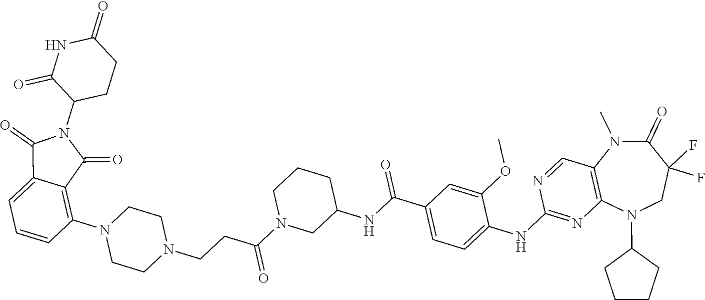 [SMILES: COc1cc(C(=O)NC2CCCN(C(=O)CCN3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 124 | 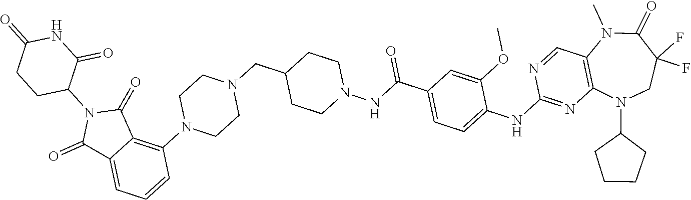 [SMILES: COc1cc(C(=O)NN2CCC(CN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 125 | 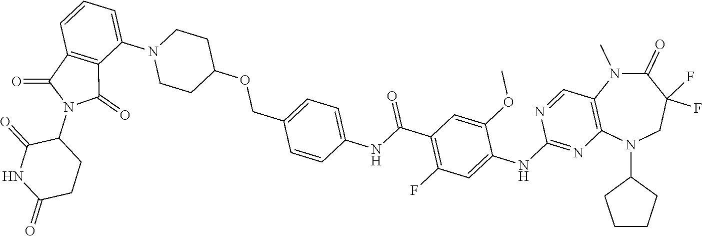 [SMILES: COc1cc(C(=O)Nc2ccc(COC3CCN(c4cccc5c4C(=O)N(C4CCC(=O)NC4=O)C5=O)CC3)cc2)c(F)cc1Nc1ncc2c(n1)N(C1CCCC1)CC(F)(F)C(=O)N2C] |
| 126 | 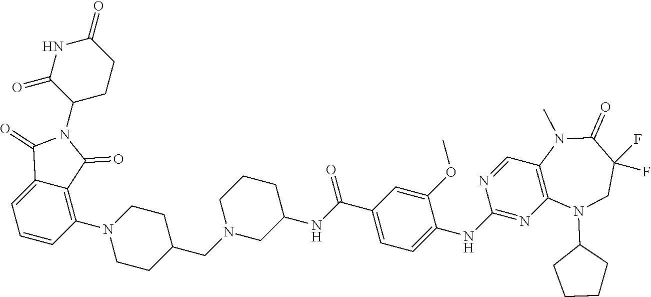 [SMILES: COc1cc(C(=O)NC2CCCN(CC3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 127 | 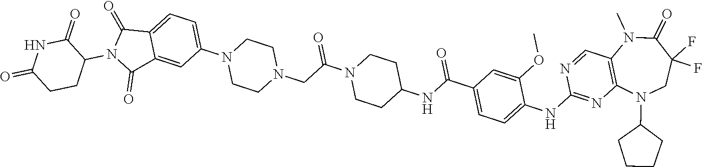 [SMILES: COc1cc(C(=O)NC2CCN(C(=O)CN3CCN(c4ccc5c(c4)C(=O)N(C4CCC(=O)NC4=O)C5=O)CC3)CC2)ccc1Nc1ncc2c(n1)N(C1CCCC1)CC(F)(F)C(=O)N2C] |
| 128 | 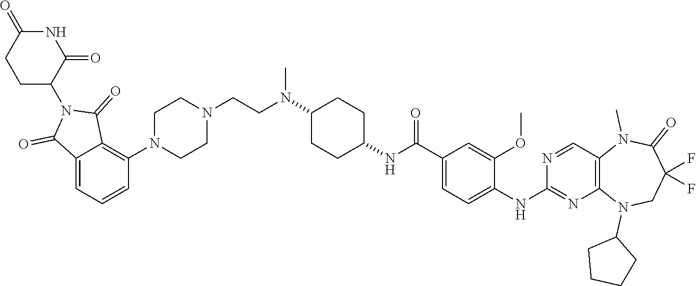 [SMILES: COc1cc(C(=O)N[C@H]2CC[C@@H](N(C)CCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 129 | 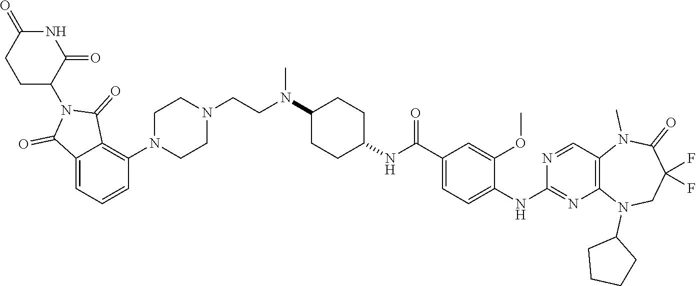 [SMILES: COc1cc(C(=O)N[C@H]2CC[C@H](N(C)CCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 130 | 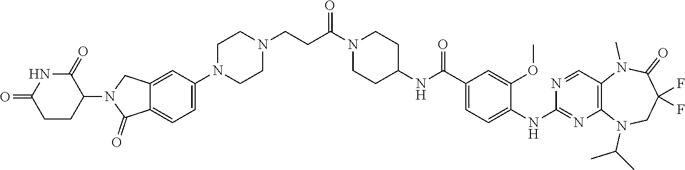 [SMILES: COc1cc(C(=O)NC2CCN(C(=O)CCN3CCN(c4ccc5c(c4)CN(C4CCC(=O)NC4=O)C5=O)CC3)CC2)ccc1Nc1ncc2c(n1)N(C(C)C)CC(F)(F)C(=O)N2C] |
| 131 | 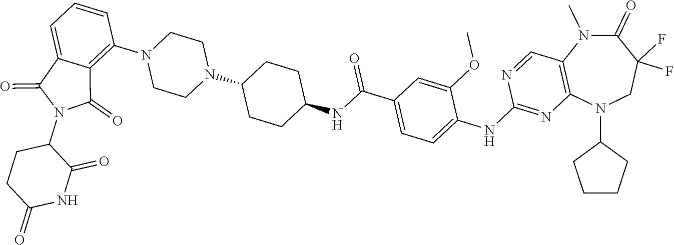 [SMILES: COc1cc(C(=O)N[C@H]2CC[C@H](N3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 132 | 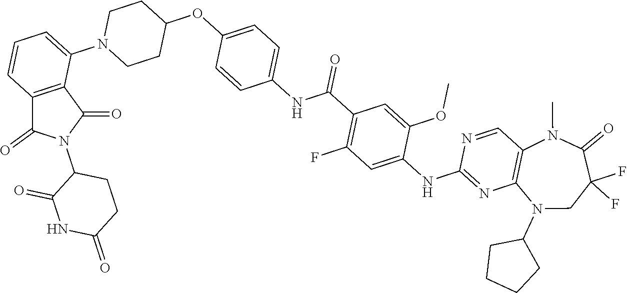 [SMILES: COc1cc(C(=O)Nc2ccc(OC3CCN(c4cccc5c4C(=O)N(C4CCC(=O)NC4=O)C5=O)CC3)cc2)c(F)cc1Nc1ncc2c(n1)N(C1CCCC1)CC(F)(F)C(=O)N2C] |
| 133 | 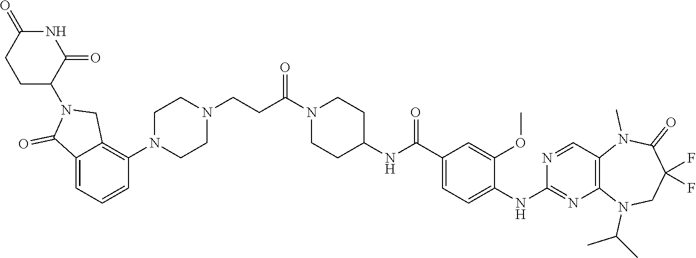 [SMILES: COc1cc(C(=O)NC2CCN(C(=O)CCN3CCN(c4cccc5c4CN(C4CCC(=O)NC4=O)C5=O)CC3)CC2)ccc1Nc1ncc2c(n1)N(C(C)C)CC(F)(F)C(=O)N2C] |
| 134 | 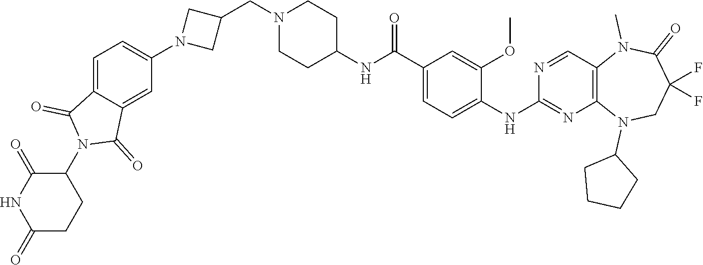 [SMILES: COc1cc(C(=O)NC2CCN(CC3CN(c4ccc5c(c4)C(=O)N(C4CCC(=O)NC4=O)C5=O)C3)CC2)ccc1Nc1ncc2c(n1)N(C1CCCC1)CC(F)(F)C(=O)N2C] |
| 135 | 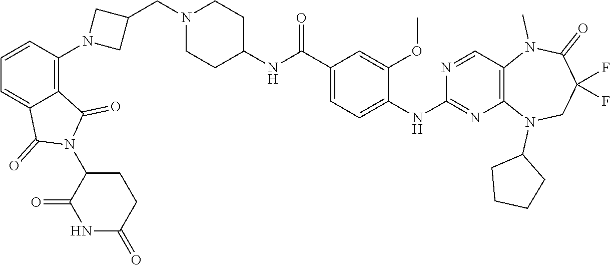 [SMILES: COc1cc(C(=O)NC2CCN(CC3CN(c4cccc5c4C(=O)N(C4CCC(=O)NC4=O)C5=O)C3)CC2)ccc1Nc1ncc2c(n1)N(C1CCCC1)CC(F)(F)C(=O)N2C] |
| 136 | 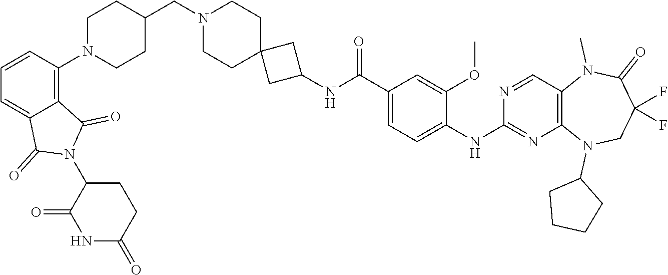 [SMILES: COc1cc(C(=O)NC2CC3(CCN(CC4CCN(c5cccc6c5C(=O)N(C5CCC(=O)NC5=O)C6=O)CC4)CC3)C2)ccc1Nc1ncc2c(n1)N(C1CCCC1)CC(F)(F)C(=O)N2C] |
| 137 | 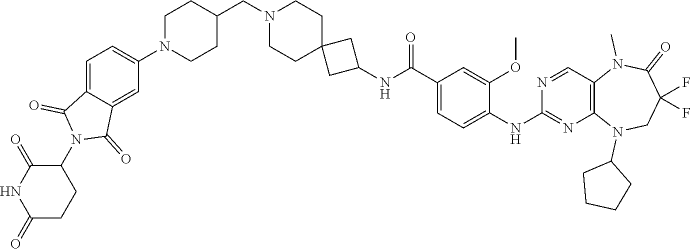 [SMILES: COc1cc(C(=O)NC2CC3(CCN(CC4CCN(c5ccc6c(c5)C(=O)N(C5CCC(=O)NC5=O)C6=O)CC4)CC3)C2)ccc1Nc1ncc2c(n1)N(C1CCCC1)CC(F)(F)C(=O)N2C] |
| 138 | 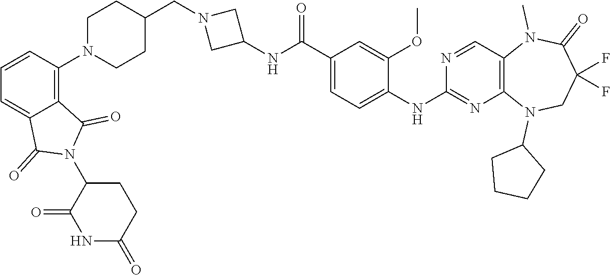 [SMILES: COc1cc(C(=O)NC2CN(CC3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 139 | 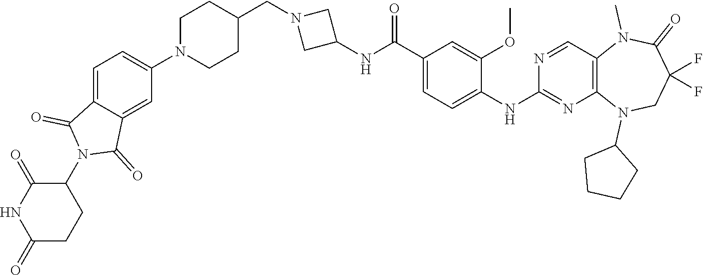 [SMILES: COc1cc(C(=O)NC2CN(CC3CCN(c4ccc5c(c4)C(=O)N(C4CCC(=O)NC4=O)C5=O)CC3)C2)ccc1Nc1ncc2c(n1)N(C1CCCC1)CC(F)(F)C(=O)N2C] |
| 140 | 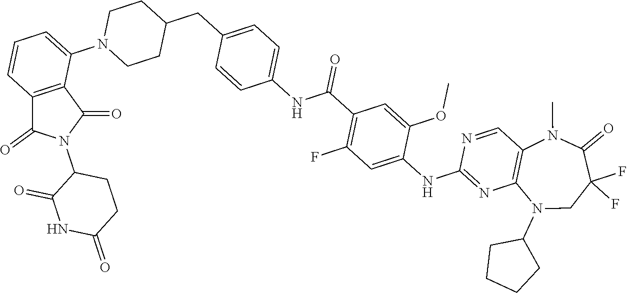 [SMILES: COc1cc(C(=O)Nc2ccc(CC3CCN(c4cccc5c4C(=O)N(C4CCC(=O)NC4=O)C5=O)CC3)cc2)c(F)cc1Nc1ncc2c(n1)N(C1CCCC1)CC(F)(F)C(=O)N2C] |
| 141 | 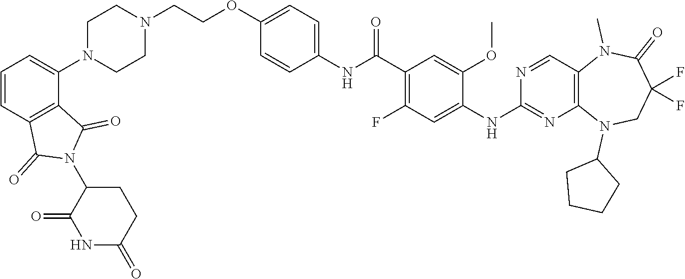 [SMILES: COc1cc(C(=O)Nc2ccc(OCCN3CCN(c4cccc5c4C(=O)N(C4CCC(=O)NC4=O)C5=O)CC3)cc2)c(F)cc1Nc1ncc2c(n1)N(C1CCCC1)CC(F)(F)C(=O)N2C] |
| 142 | 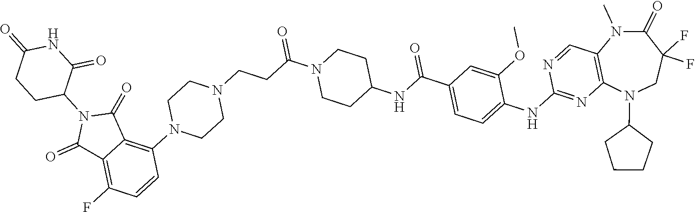 [SMILES: COc1cc(C(=O)NC2CCN(C(=O)CCN3CCN(c4ccc(F)c5c4C(=O)N(C4CCC(=O)NC4=O)C5=O)CC3)CC2)ccc1Nc1ncc2c(n1)N(C1CCCC1)CC(F)(F)C(=O)N2C] |
| 143 | 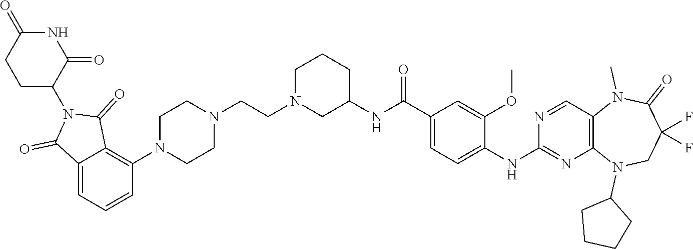 [SMILES: COc1cc(C(=O)NC2CCCN(CCN3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 144 | 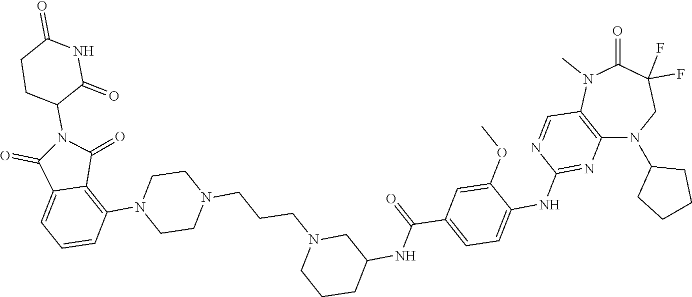 [SMILES: COc1cc(C(=O)NC2CCCN(CCCN3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 145 | 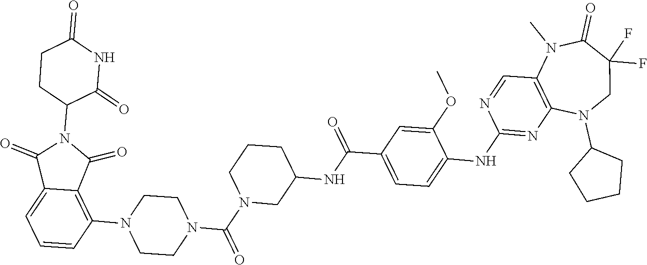 [SMILES: COc1cc(C(=O)NC2CCCN(C(=O)N3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 146 | 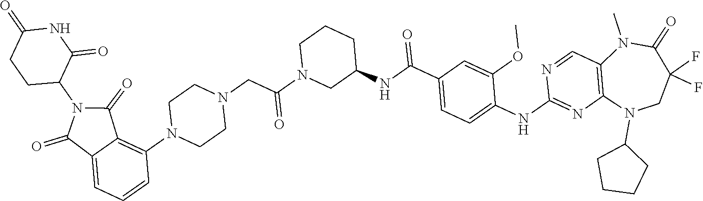 [SMILES: COc1cc(C(=O)N[C@@H]2CCCN(C(=O)CN3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 147 | 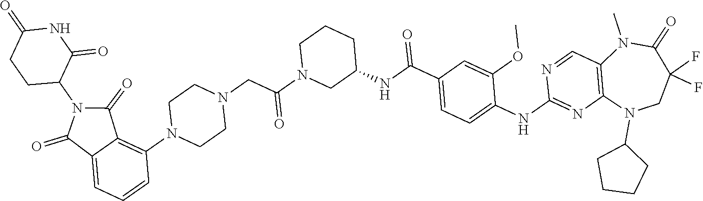 [SMILES: COc1cc(C(=O)N[C@H]2CCCN(C(=O)CN3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 148 | 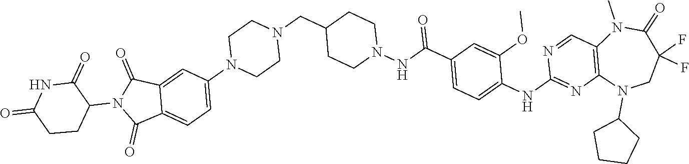 [SMILES: COc1cc(C(=O)NN2CCC(CN3CCN(c4ccc5c(c4)C(=O)N(C4CCC(=O)NC4=O)C5=O)CC3)CC2)ccc1Nc1ncc2c(n1)N(C1CCCC1)CC(F)(F)C(=O)N2C] |
| 149 | 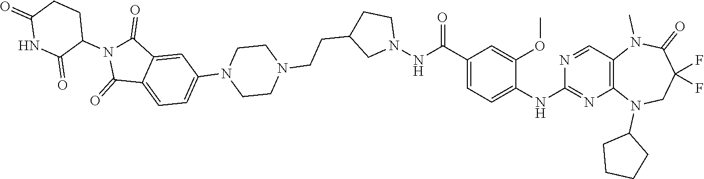 [SMILES: COc1cc(C(=O)NN2CCC(CCN3CCN(c4ccc5c(c4)C(=O)N(C4CCC(=O)NC4=O)C5=O)CC3)C2)ccc1Nc1ncc2c(n1)N(C1CCCC1)CC(F)(F)C(=O)N2C] |
| 150 | 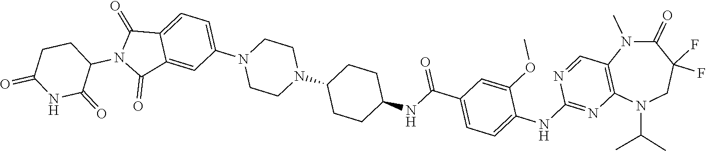 [SMILES: COc1cc(C(=O)N[C@H]2CC[C@H](N3CCN(c4ccc5c(c4)C(=O)N(C4CCC(=O)NC4=O)C5=O)CC3)CC2)ccc1Nc1ncc2c(n1)N(C(C)C)CC(F)(F)C(=O)N2C] |
| 151 | 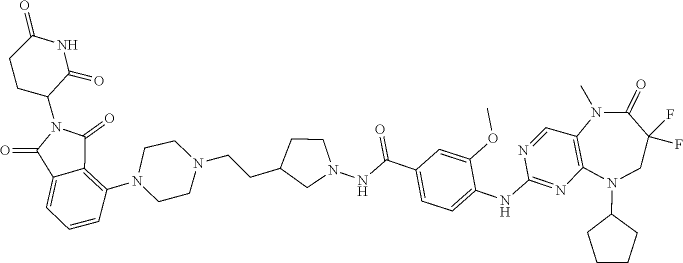 [SMILES: COc1cc(C(=O)NN2CCC(CCN3CCN(c4cccc5c4CC(=O)N(C4CCC(=O)NC4=O)C5=O)CC3)C2)ccc1Nc1ncc2c(n1)N(C1CCCC1)CC(F)(F)C(=O)N2C] |
| 152 | 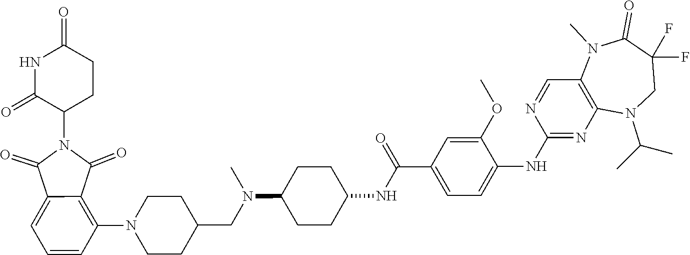 [SMILES: COc1cc(C(=O)N[C@H]2CC[C@H](N(C)CC3CCN(c4cccc5c4C(=O)N(C4CCC(=O)NC4=O)C5=O)CC3)CC2)ccc1Nc1ncc2c(n1)N(C(C)C)CC(F)(F)C(=O)N2C] |
| 153 | 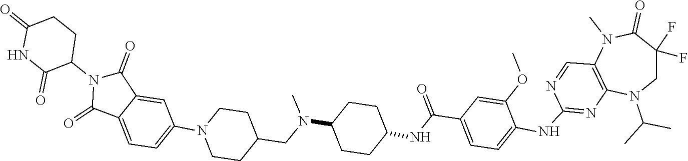 [SMILES: COc1cc(C(=O)N[C@H]2CC[C@H](N(C)CC3CCN(c4ccc5c(c4)C(=O)N(C4CCC(=O)NC4=O)C5=O)CC3)CC2)ccc1Nc1ncc2c(n1)N(C(C)C)CC(F)(F)C(=O)N2C] |
| 154 | 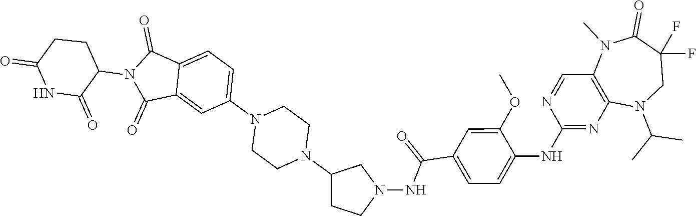 [SMILES: COc1cc(C(=O)NN2CCC(N3CCN(c4ccc5c(c4)C(=O)N(C4CCC(=O)NC4=O)C5=O)CC3)C2)ccc1Nc1ncc2c(n1)N(C(C)C)CC(F)(F)C(=O)N2C] |
| 155 | 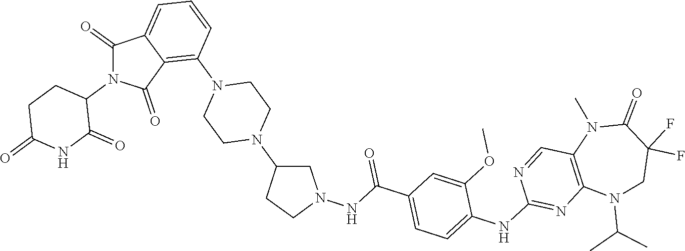 [SMILES: COc1cc(C(=O)NN2CCC(N3CCN(c4cccc5c4C(=O)N(C4CCC(=O)NC4=O)C5=O)CC3)C2)ccc1Nc1ncc2c(n1)N(C(C)C)CC(F)(F)C(=O)N2C] |
| 156 | 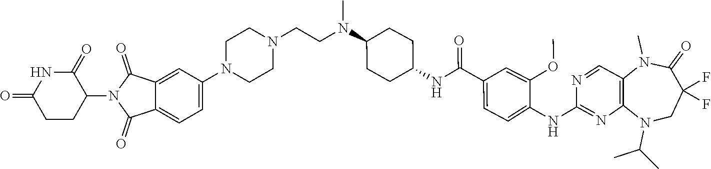 [SMILES: COc1cc(C(=O)N[C@H]2CC[C@H](N(C)CCN3CCN(c4ccc5c(c4)C(=O)N(C4CCC(=O)NC4=O)C5=O)CC3)CC2)ccc1Nc1ncc2c(n1)N(C(C)C)CC(F)(F)C(=O)N2C] |
| 157 | 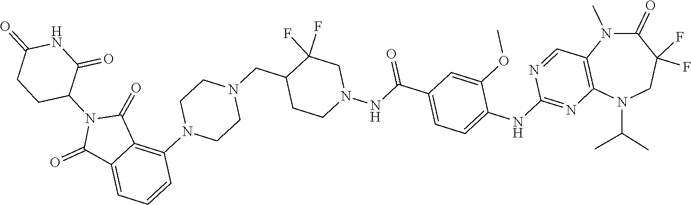 [SMILES: COc1cc(C(=O)NN2CCC(CN3CCN(c4cccc5c4C(=O)N(C4CCC(=O)NC4=O)C5=O)CC3)C(F)(F)C2)ccc1Nc1ncc2c(n1)N(C(C)C)CC(F)(F)C(=O)N2C] |
| 158 | 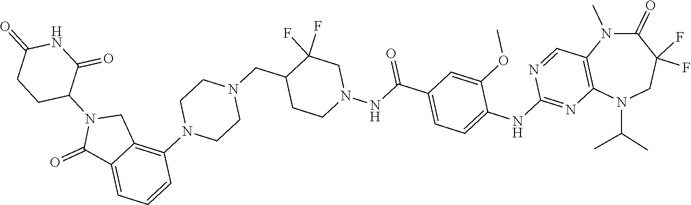 [SMILES: COc1cc(C(=O)NN2CCC(CN3CCN(c4cccc5c4CN(C4CCC(=O)NC4=O)C5=O)CC3)C(F)(F)C2)ccc1Nc1ncc2c(n1)N(C(C)C)CC(F)(F)C(=O)N2C] |
| 159 | 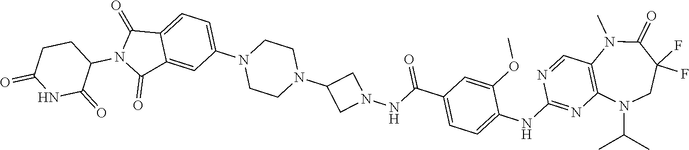 [SMILES: COc1cc(C(=O)NN2CC(N3CCN(c4ccc5c(c4)C(=O)N(C4CCC(=O)NC4=O)C5=O)CC3)C2)ccc1Nc1ncc2c(n1)N(C(C)C)CC(F)(F)C(=O)N2C] |
| 160 | 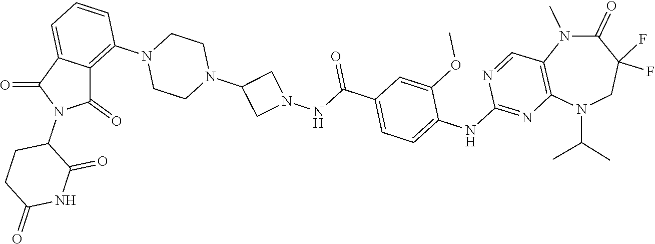 [SMILES: COc1cc(C(=O)NN2CC(N3CCN(c4cccc5c4C(=O)N(C4CCC(=O)NC4=O)C5=O)CC3)C2)ccc1Nc1ncc2c(n1)N(C(C)C)CC(F)(F)C(=O)N2C] |
| 161 | 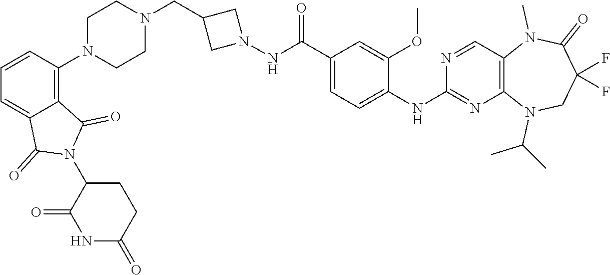 [SMILES: COc1cc(C(=O)NN2CC(CN3CCN(c4cccc5c4C(=O)N(C4CCC(=O)NC4=O)C5=O)CC3)C2)ccc1Nc1ncc2c(n1)N(C(C)C)CC(F)(F)C(=O)N2C] |
| 162 | 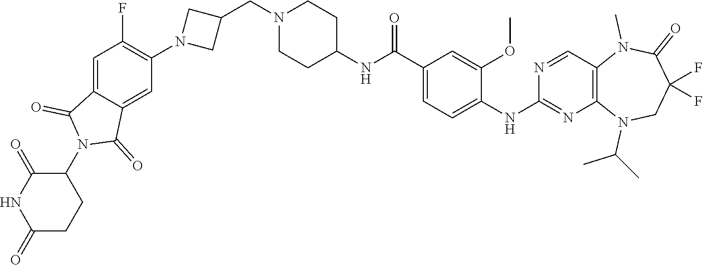 [SMILES: COc1cc(C(=O)NC2CCN(CC3CN(c4cc5c(cc4F)C(=O)N(C4CCC(=O)NC4=O)C5=O)C3)CC2)ccc1Nc1ncc2c(n1)N(C(C)C)CC(F)(F)C(=O)N2C] |
| 163 | 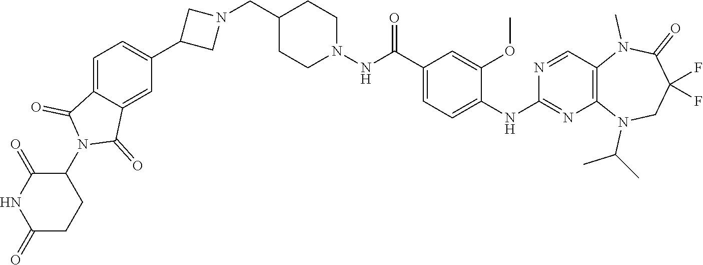 [SMILES: COc1cc(C(=O)NN2CCC(CN3CC(c4ccc5c(c4)C(=O)N(C4CCC(=O)NC4=O)C5=O)C3)CC2)ccc1Nc1ncc2c(n1)N(C(C)C)CC(F)(F)C(=O)N2C] |
| 164 | 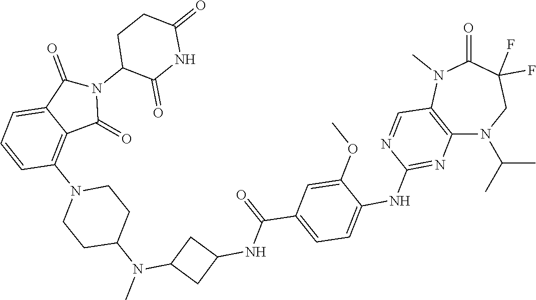 [SMILES: COc1cc(C(=O)NC2CC(N(C)C3CCN(c4cccc5c4C(=O)N(C4CCC(=O)NC4=O)C5=O)CC3)C2)ccc1Nc1ncc2c(n1)N(C(C)C)CC(F)(F)C(=O)N2C] |
| 165 | 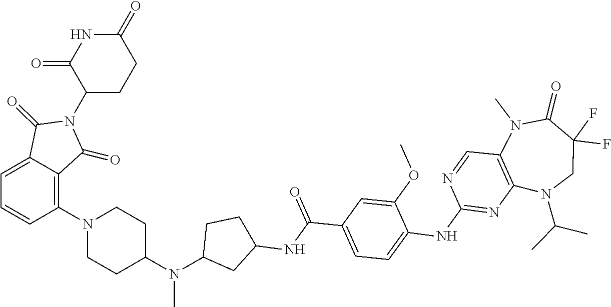 [SMILES: COc1cc(C(=O)NC2CCC(N(C)C3CCN(c4cccc5c4C(=O)N(C4CCC(=O)NC4=O)C5=O)CC3)C2)ccc1Nc1ncc2c(n1)N(C(C)C)CC(F)(F)C(=O)N2C] |
| 166 | 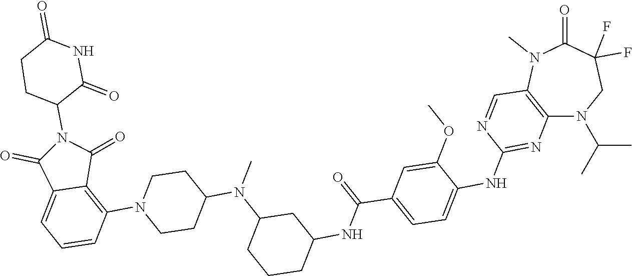 [SMILES: COc1cc(C(=O)NC2CCCC(N(C)C3CCN(c4cccc5c4C(=O)N(C4CCC(=O)NC4=O)C5=O)CC3)C2)ccc1Nc1ncc2c(n1)N(C(C)C)CC(F)(F)C(=O)N2C] |
| 167 | 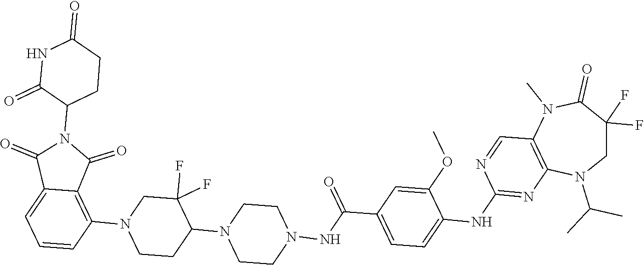 [SMILES: COc1cc(C(=O)NN2CCN(C3CCN(c4cccc5c4C(=O)N(C4CCC(=O)NC4=O)C5=O)CC3(F)F)CC2)ccc1Nc1ncc2c(n1)N(C(C)C)CC(F)(F)C(=O)N2C] |
| 168 | 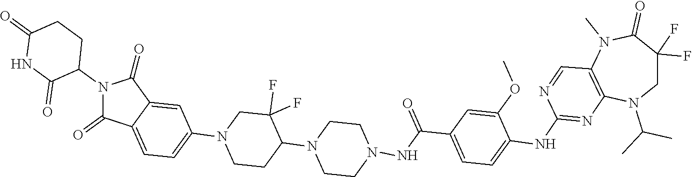 [SMILES: COc1cc(C(=O)NN2CCN(C3CCN(c4ccc5c(c4)C(=O)N(C4CCC(=O)NC4=O)C5=O)CC3(F)F)CC2)ccc1Nc1ncc2c(n1)N(C(C)C)CC(F)(F)C(=O)N2C] |
| 169 | 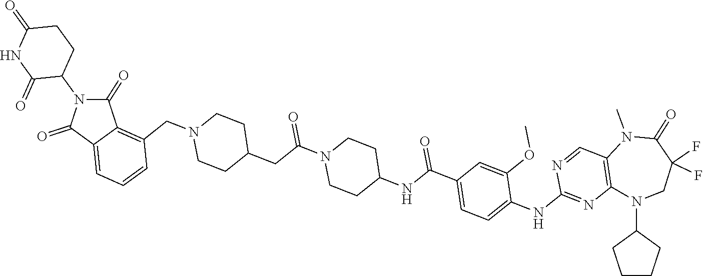 [SMILES: COc1cc(C(=O)NC2CCN(C(=O)CC3CCN(Cc4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 170 | 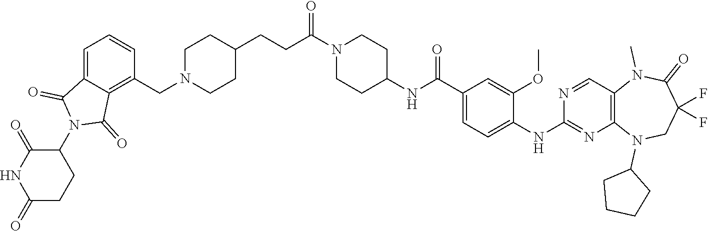 [SMILES: COc1cc(C(=O)NC2CCN(C(=O)CCC3CCN(Cc4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 171 | 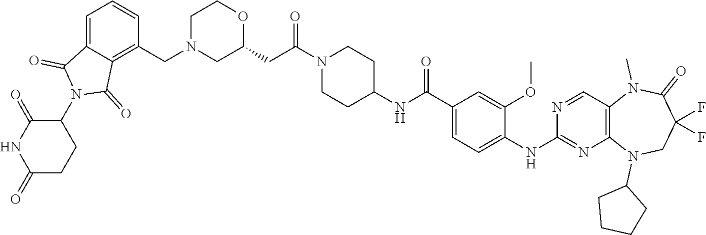 [SMILES: COc1cc(C(=O)NC2CCN(C(=O)C[C@@H]3CN(Cc4cccc5c4C(=O)N(C4CCC(=O)NC4=O)C5=O)CCO3)CC2)ccc1Nc1ncc2c(n1)N(C1CCCC1)CC(F)(F)C(=O)N2C] |
| 172 | 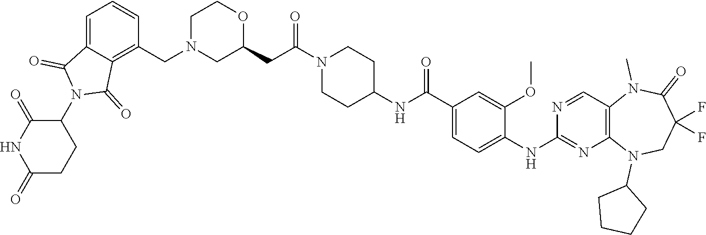 [SMILES: COc1cc(C(=O)NC2CCN(C(=O)C[C@H]3CN(Cc4cccc5c4C(=O)N(C4CCC(=O)NC4=O)C5=O)CCO3)CC2)ccc1Nc1ncc2c(n1)N(C1CCCC1)CC(F)(F)C(=O)N2C] |
| 173 | 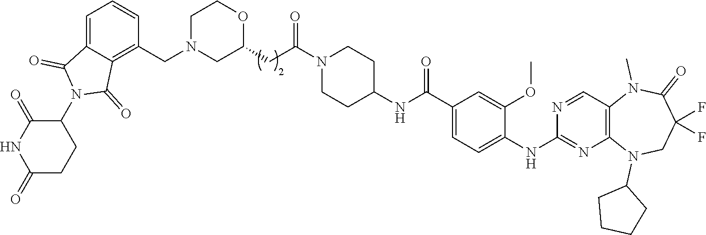 |
| 174 | 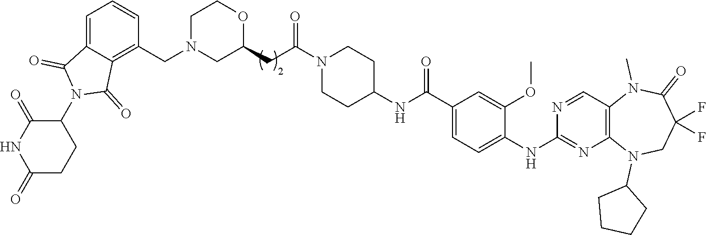 |
| 175 | 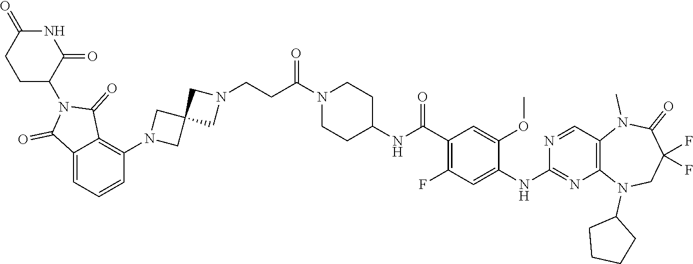 |
| 176 | 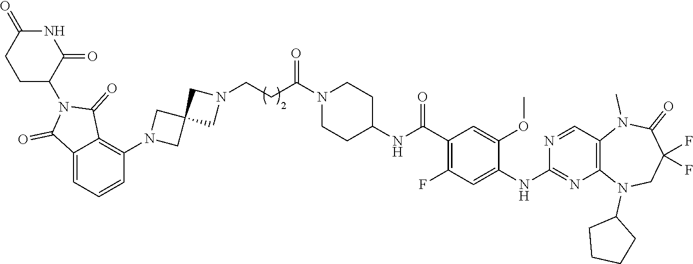 |
| 177 | 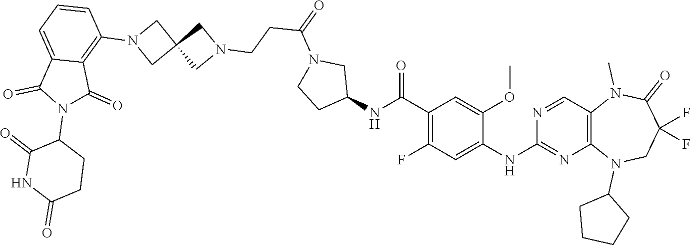 |
| 178 | 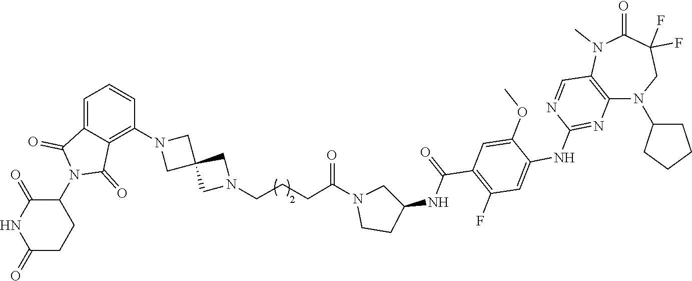 |
| 179 | 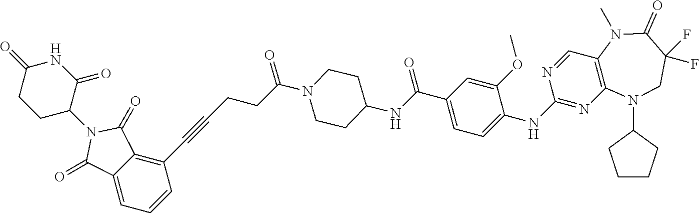 [SMILES: COc1cc(C(=O)NC2CCN(C(=O)CCC#Cc3cccc4c3C(=O)N(C3CCC(=O)NC3=O)C4=O)CC2)ccc1Nc1ncc2c(n1)N(C1CCCC1)CC(F)(F)C(=O)N2C] |
| 180 | 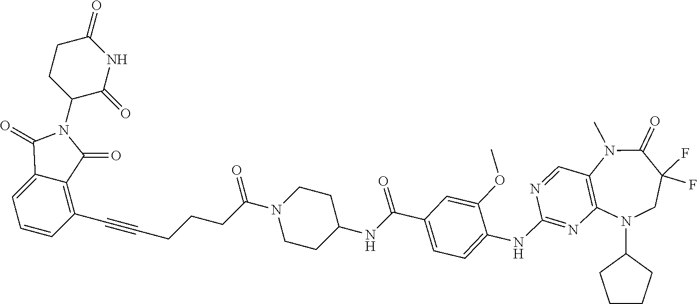 [SMILES: COc1cc(C(=O)NC2CCN(C(=O)CCCC#Cc3cccc4c3C(=O)N(C3CCC(=O)NC3=O)C4=O)CC2)ccc1Nc1ncc2c(n1)N(C1CCCC1)CC(F)(F)C(=O)N2C] |
| 181 | 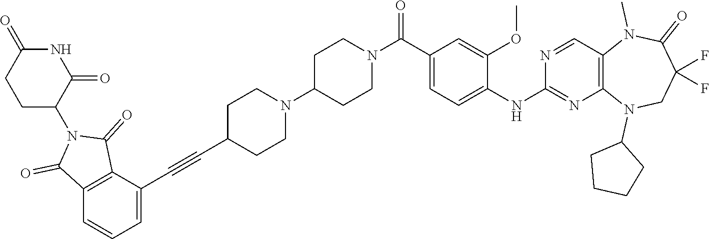 [SMILES: COc1cc(C(=O)N2CCC(N3CCC(C#Cc4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 182 | 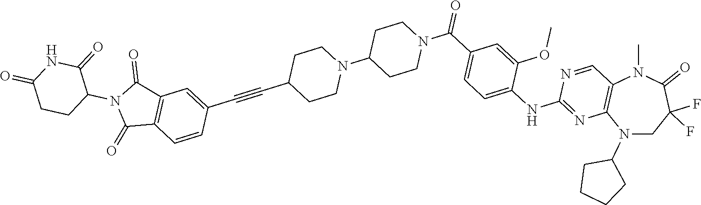 [SMILES: COc1cc(C(=O)N2CCC(N3CCC(C#Cc4ccc5c(c4)C(=O)N(C4CCC(=O)NC4=O)C5=O)CC3)CC2)ccc1Nc1ncc2c(n1)N(C1CCCC1)CC(F)(F)C(=O)N2C] |
| 183 | 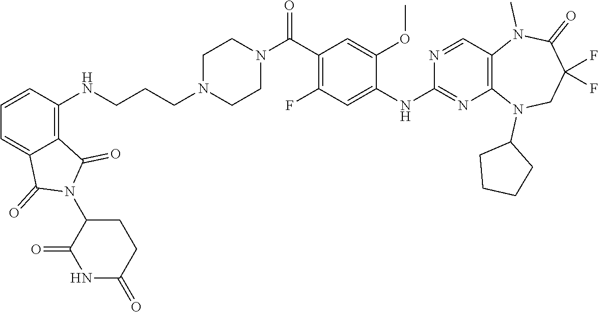 [SMILES: COc1cc(C(=O)N2CCN(CCCNc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 184 | 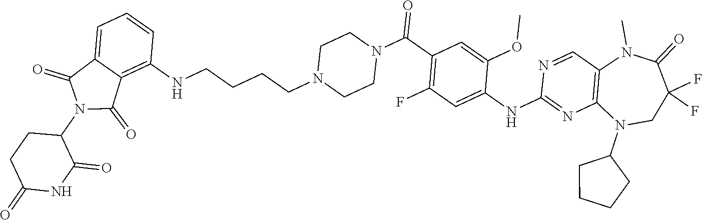 [SMILES: COc1cc(C(=O)N2CCN(CCCCNc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 185 | 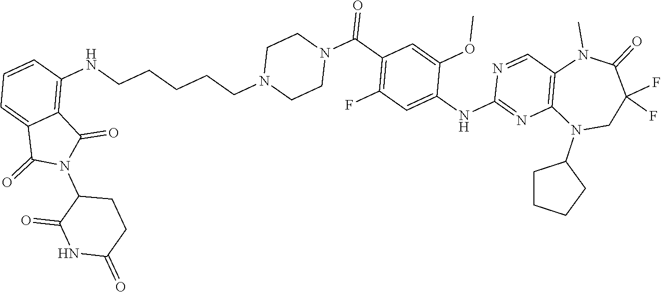 [SMILES: COc1cc(C(=O)N2CCN(CCCCCNc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 186 | 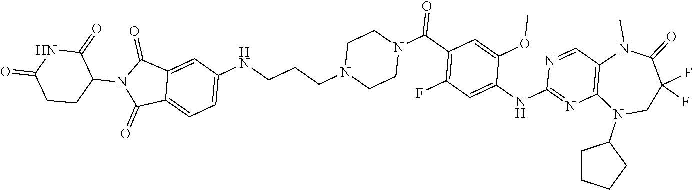 [SMILES: COc1cc(C(=O)N2CCN(CCCNc3ccc4c(c3)C(=O)N(C3CCC(=O)NC3=O)C4=O)CC2)c(F)cc1Nc1ncc2c(n1)N(C1CCCC1)CC(F)(F)C(=O)N2C] |
| 187 | 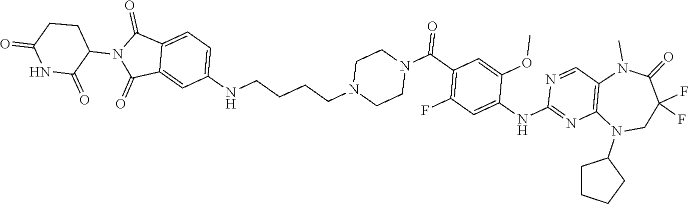 [SMILES: COc1cc(C(=O)N2CCN(CCCCNc3ccc4c(c3)C(=O)N(C3CCC(=O)NC3=O)C4=O)CC2)c(F)cc1Nc1ncc2c(n1)N(C1CCCC1)CC(F)(F)C(=O)N2C] |
| 188 | 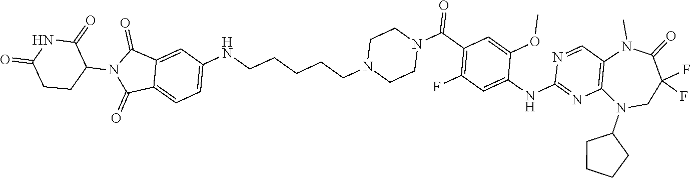 [SMILES: COc1cc(C(=O)N2CCN(CCCCCNc3ccc4c(c3)C(=O)N(C3CCC(=O)NC3=O)C4=O)CC2)c(F)cc1Nc1ncc2c(n1)N(C1CCCC1)CC(F)(F)C(=O)N2C] |
| 189 | 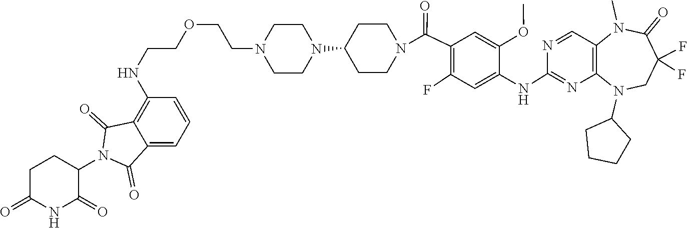 |
| 190 | 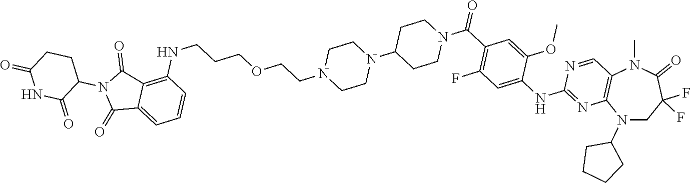 [SMILES: COc1cc(C(=O)N2CCC(N3CCN(CCOCCCNc4cccc5c4C(=O)N(C4CCC(=O)NC4=O)C5=O)CC3)CC2)c(F)cc1Nc1ncc2c(n1)N(C1CCCC1)CC(F)(F)C(=O)N2C] |
| 191 | 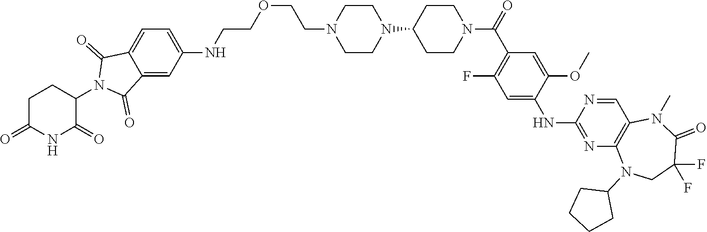 |
| 192 | 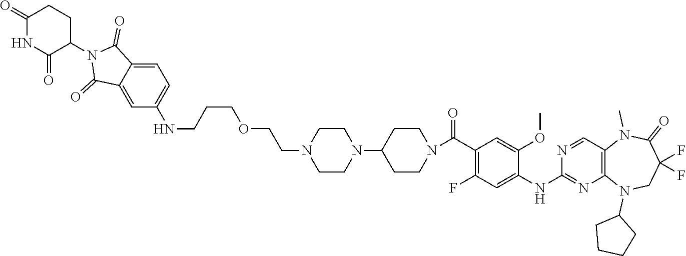 [SMILES: COc1cc(C(=O)N2CCC(N3CCN(CCOCCCNc4ccc5c(c4)C(=O)N(C4CCC(=O)NC4=O)C5=O)CC3)CC2)c(F)cc1Nc1ncc2c(n1)N(C1CCCC1)CC(F)(F)C(=O)N2C] |
| 193 | 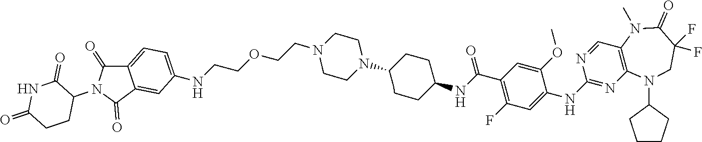 |
| 194 | 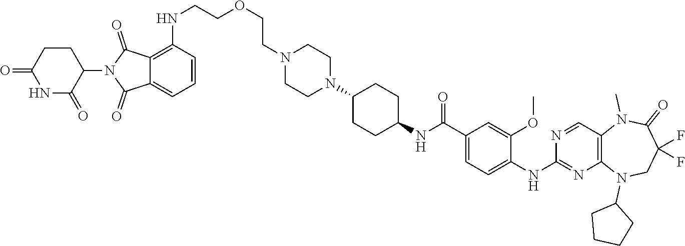 [SMILES: COc1cc(C(=O)N[C@H]2CC[C@H](N3CCN(CCOCCNc4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 195 | 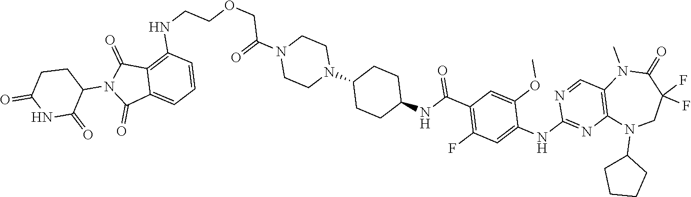 [SMILES: COc1cc(C(=O)N[C@H]2CC[C@H](N3CCN(C(=O)COCCNc4cccc5c4C(=O)N(C4CCC(=O)NC4=O)C5=O)CC3)CC2)c(F)cc1Nc1ncc2c(n1)N(C1CCCC1)CC(F)(F)C(=O)N2C] |
| 196 | 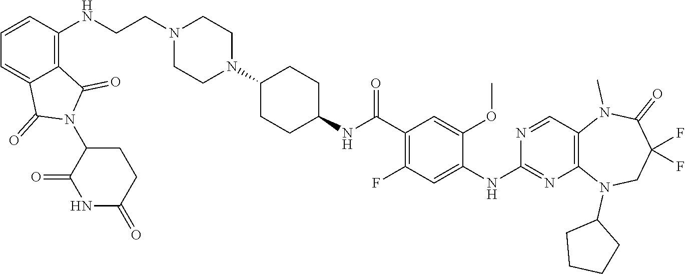 [SMILES: COc1cc(C(=O)N[C@H]2CC[C@H](N3CCN(CCNc4cccc5c4C(=O)N(C4CCC(=O)NC4=O)C5=O)CC3)CC2)c(F)cc1Nc1ncc2c(n1)N(C1CCCC1)CC(F)(F)C(=O)N2C] |
| 197 | 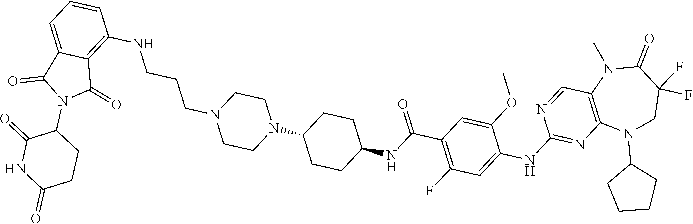 [SMILES: COc1cc(C(=O)N[C@H]2CC[C@H](N3CCN(CCCNc4cccc5c4C(=O)N(C4CCC(=O)NC4=O)C5=O)CC3)CC2)c(F)cc1Nc1ncc2c(n1)N(C1CCCC1)CC(F)(F)C(=O)N2C] |
| 198 | 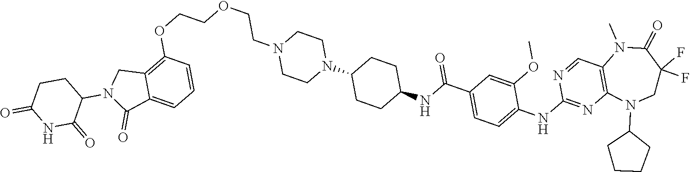 [SMILES: COc1cc(C(=O)N[C@H]2CC[C@H](N3CCN(CCOCCOc4cccc5c4CN(C4CCC(=O)NC4=O)C5=O)CC3)CC2)ccc1Nc1ncc2c(n1)N(C1CCCC1)CC(F)(F)C(=O)N2C] |
| 199 | 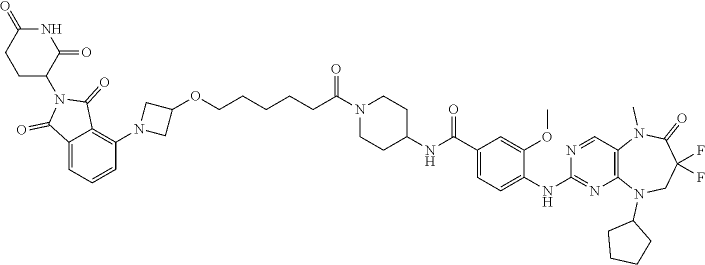 [SMILES: COc1cc(C(=O)NC2CCN(C(=O)CCCCCOC3CN(c4cccc5c4C(=O)N(C4CCC(=O)NC4=O)C5=O)C3)CC2)ccc1Nc1ncc2c(n1)N(C1CCCC1)CC(F)(F)C(=O)N2C] |
| 200 | 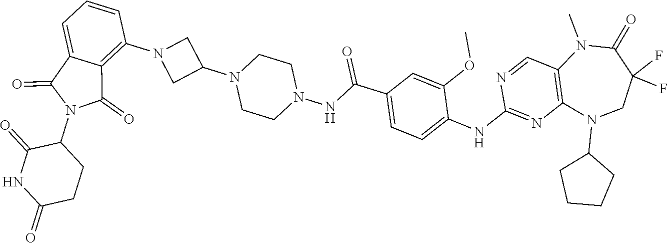 [SMILES: COc1cc(C(=O)NN2CCN(C3CN(c4cccc5c4C(=O)N(C4CCC(=O)NC4=O)C5=O)C3)CC2)ccc1Nc1ncc2c(n1)N(C1CCCC1)CC(F)(F)C(=O)N2C] |
| 201 | 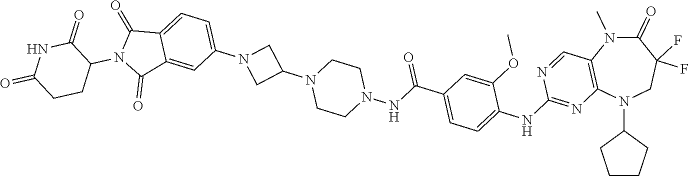 [SMILES: COc1cc(C(=O)NN2CCN(C3CN(c4ccc5c(c4)C(=O)N(C4CCC(=O)NC4=O)C5=O)C3)CC2)ccc1Nc1ncc2c(n1)N(C1CCCC1)CC(F)(F)C(=O)N2C] |
| 202 | 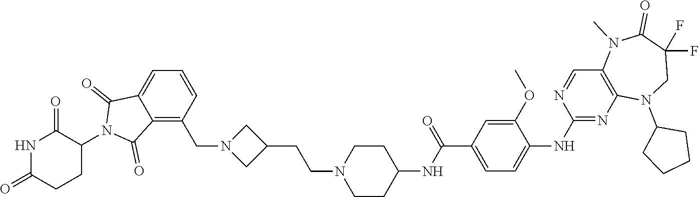 [SMILES: COc1cc(C(=O)NC2CCN(CCC3CN(Cc4cccc5c4C(=O)N(C4CCC(=O)NC4=O)C5=O)C3)CC2)ccc1Nc1ncc2c(n1)N(C1CCCC1)CC(F)(F)C(=O)N2C] |
| 203 | 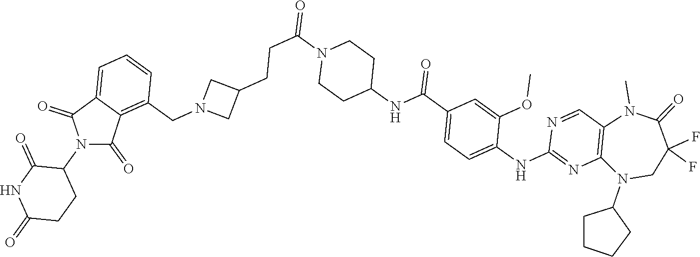 [SMILES: COc1cc(C(=O)NC2CCN(C(=O)CCC3CN(Cc4cccc5c4C(=O)N(C4CCC(=O)NC4=O)C5=O)C3)CC2)ccc1Nc1ncc2c(n1)N(C1CCCC1)CC(F)(F)C(=O)N2C] |
| 204 | 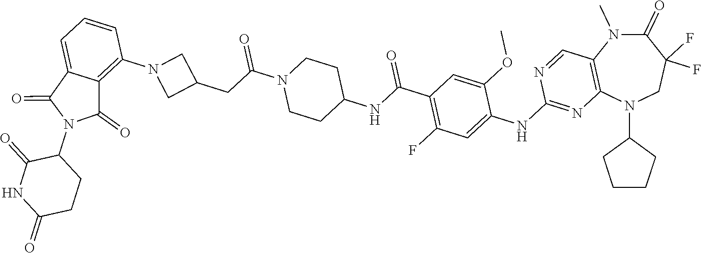 [SMILES: COc1cc(C(=O)NC2CCN(C(=O)CC3CN(c4cccc5c4C(=O)N(C4CCC(=O)NC4=O)C5=O)C3)CC2)c(F)cc1Nc1ncc2c(n1)N(C1CCCC1)CC(F)(F)C(=O)N2C] |
| 205 | 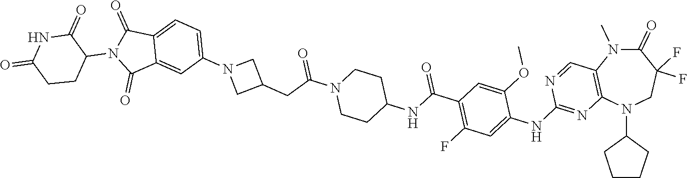 [SMILES: COc1cc(C(=O)NC2CCN(C(=O)CC3CN(c4ccc5c(c4)C(=O)N(C4CCC(=O)NC4=O)C5=O)C3)CC2)c(F)cc1Nc1ncc2c(n1)N(C1CCCC1)CC(F)(F)C(=O)N2C] |
| 206 | 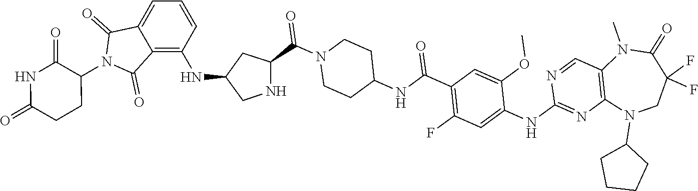 [SMILES: COc1cc(C(=O)NC2CCN(C(=O)[C@@H]3C[C@H](Nc4cccc5c4C(=O)N(C4CCC(=O)NC4=O)C5=O)CN3)CC2)c(F)cc1Nc1ncc2c(n1)N(C1CCCC1)CC(F)(F)C(=O)N2C] |
| 207 | 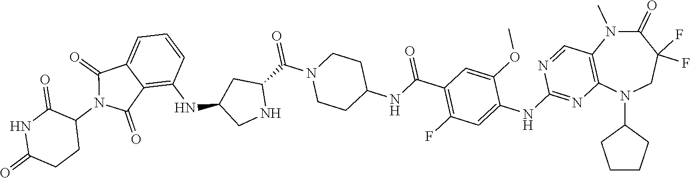 [SMILES: COc1cc(C(=O)NC2CCN(C(=O)[C@H]3C[C@H](Nc4cccc5c4C(=O)N(C4CCC(=O)NC4=O)C5=O)CN3)CC2)c(F)cc1Nc1ncc2c(n1)N(C1CCCC1)CC(F)(F)C(=O)N2C] |
| 208 | 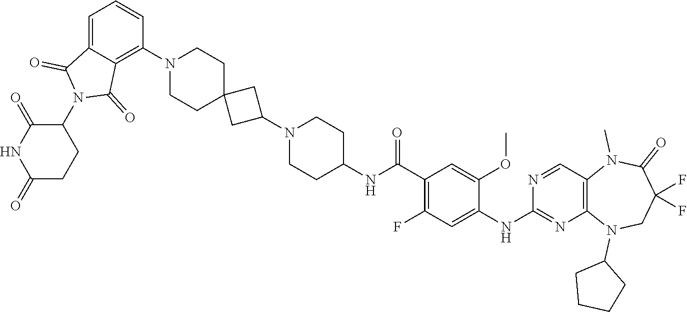 [SMILES: COc1cc(C(=O)NC2CCN(C3CC4(CCN(c5cccc6c5C(=O)N(C5CCC(=O)NC5=O)C6=O)CC4)C3)CC2)c(F)cc1Nc1ncc2c(n1)N(C1CCCC1)CC(F)(F)C(=O)N2C] |
| 209 | 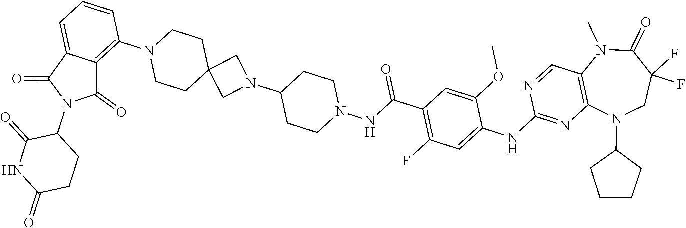 [SMILES: COc1cc(C(=O)NN2CCC(N3CC4(CCN(c5cccc6c5C(=O)N(C5CCC(=O)NC5=O)C6=O)CC4)C3)CC2)c(F)cc1Nc1ncc2c(n1)N(C1CCCC1)CC(F)(F)C(=O)N2C] |
| 210 | 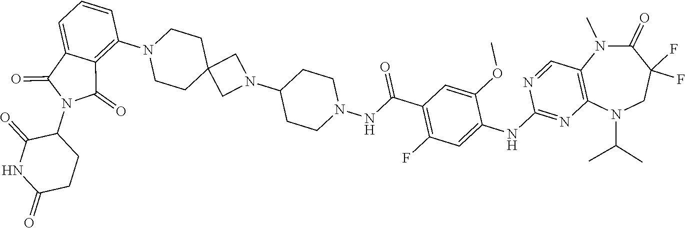 [SMILES: COc1cc(C(=O)NN2CCC(N3CC4(CCN(c5cccc6c5C(=O)N(C5CCC(=O)NC5=O)C6=O)CC4)C3)CC2)c(F)cc1Nc1ncc2c(n1)N(C(C)C)CC(F)(F)C(=O)N2C] |
| 211 | 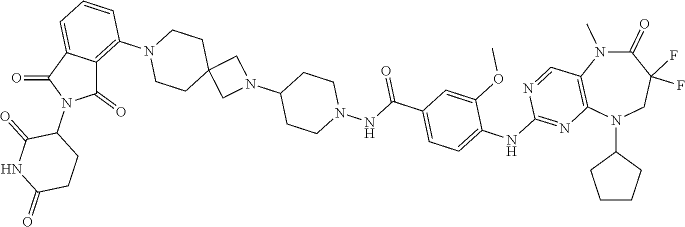 [SMILES: COc1cc(C(=O)NN2CCC(N3CC4(CCN(c5cccc6c5C(=O)N(C5CCC(=O)NC5=O)C6=O)CC4)C3)CC2)ccc1Nc1ncc2c(n1)N(C1CCCC1)CC(F)(F)C(=O)N2C] |
| 212 | 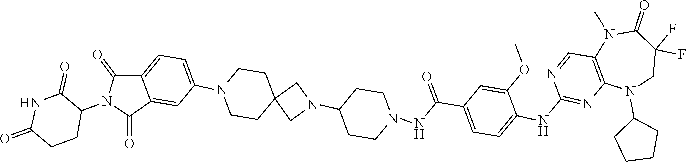 [SMILES: COc1cc(C(=O)NN2CCC(N3CC4(CCN(c5ccc6c(c5)C(=O)N(C5CCC(=O)NC5=O)C6=O)CC4)C3)CC2)ccc1Nc1ncc2c(n1)N(C1CCCC1)CC(F)(F)C(=O)N2C] |
| 213 | 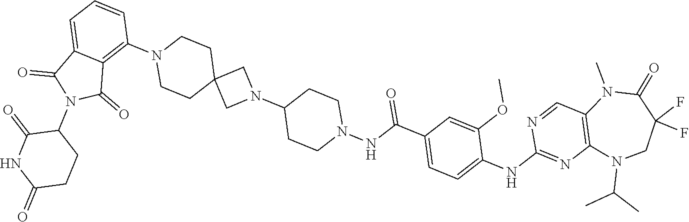 [SMILES: COc1cc(C(=O)NN2CCC(N3CC4(CCN(c5cccc6c5C(=O)N(C5CCC(=O)NC5=O)C6=O)CC4)C3)CC2)ccc1Nc1ncc2c(n1)N(C(C)C)CC(F)(F)C(=O)N2C] |
| 214 | 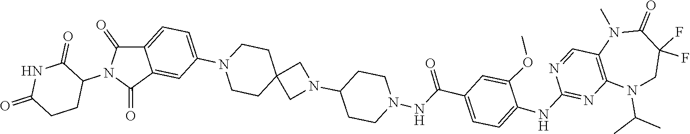 [SMILES: COc1cc(C(=O)NN2CCC(N3CC4(CCN(c5ccc6c(c5)C(=O)N(C5CCC(=O)NC5=O)C6=O)CC4)C3)CC2)ccc1Nc1ncc2c(n1)N(C(C)C)CC(F)(F)C(=O)N2C] |
| 215 | 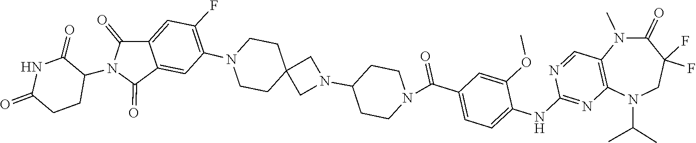 [SMILES: COc1cc(C(=O)N2CCC(N3CC4(CCN(c5cc6c(cc5F)C(=O)N(C5CCC(=O)NC5=O)C6=O)CC4)C3)CC2)ccc1Nc1ncc2c(n1)N(C(C)C)CC(F)(F)C(=O)N2C] |
| 216 | 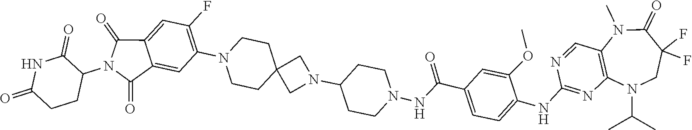 [SMILES: COc1cc(C(=O)NN2CCC(N3CC4(CCN(c5cc6c(cc5F)C(=O)N(C5CCC(=O)NC5=O)C6=O)CC4)C3)CC2)ccc1Nc1ncc2c(n1)N(C(C)C)CC(F)(F)C(=O)N2C] |
| 217 | 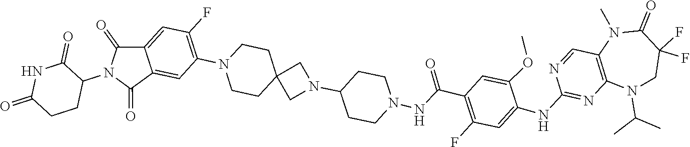 [SMILES: COc1cc(C(=O)NN2CCC(N3CC4(CCN(c5cc6c(cc5F)C(=O)N(C5CCC(=O)NC5=O)C6=O)CC4)C3)CC2)c(F)cc1Nc1ncc2c(n1)N(C(C)C)CC(F)(F)C(=O)N2C] |
| 218 | 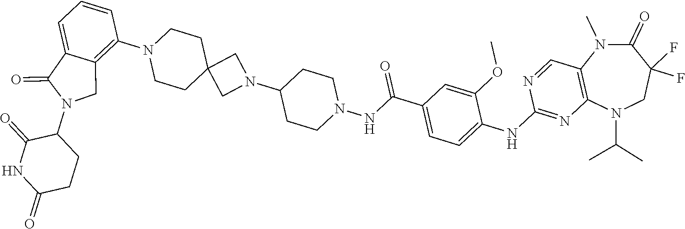 [SMILES: COc1cc(C(=O)NN2CCC(N3CC4(CCN(c5cccc6c5CN(C5CCC(=O)NC5=O)C6=O)CC4)C3)CC2)ccc1Nc1ncc2c(n1)N(C(C)C)CC(F)(F)C(=O)N2C] |
| 219 | 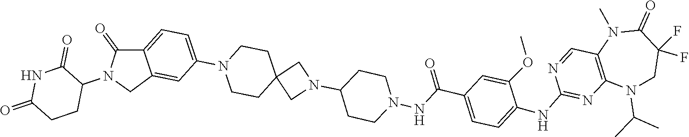 [SMILES: COc1cc(C(=O)NN2CCC(N3CC4(CCN(c5ccc6c(c5)CN(C5CCC(=O)NC5=O)C6=O)CC4)C3)CC2)ccc1Nc1ncc2c(n1)N(C(C)C)CC(F)(F)C(=O)N2C] |
| 220 | 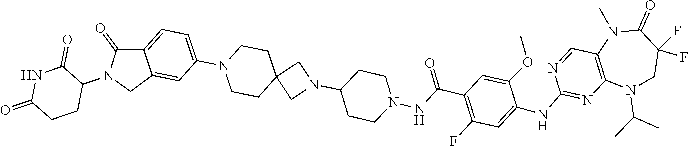 [SMILES: COc1cc(C(=O)NN2CCC(N3CC4(CCN(c5ccc6c(c5)CN(C5CCC(=O)NC5=O)C6=O)CC4)C3)CC2)c(F)cc1Nc1ncc2c(n1)N(C(C)C)CC(F)(F)C(=O)N2C] |
| 221 | 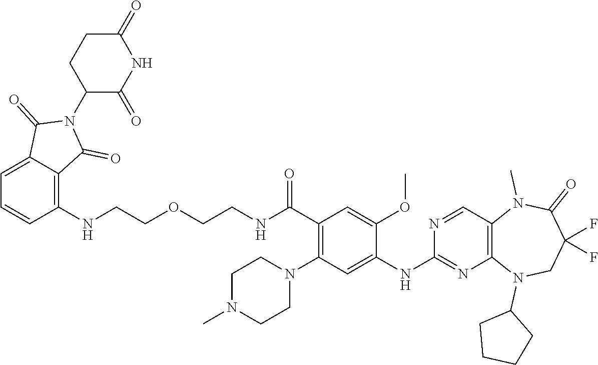 [SMILES: COc1cc(C(=O)NCCOCCNc2cccc3c2C(=O)N(C2CCC(=O)NC2=O)C3=O)c(N2CCN(C)CC2)cc1Nc1ncc2c(n1)N(C1CCCC1)CC(F)(F)C(=O)N2C] |
| 222 | 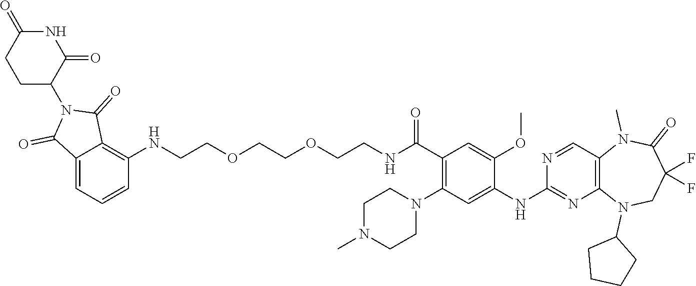 [SMILES: COc1cc(C(=O)NCCOCCOCCNc2cccc3c2C(=O)N(C2CCC(=O)NC2=O)C3=O)c(N2CCN(C)CC2)cc1Nc1ncc2c(n1)N(C1CCCC1)CC(F)(F)C(=O)N2C] |
| 223 | 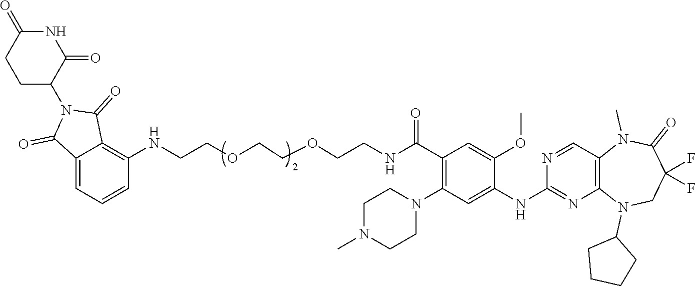 |
| 224 | 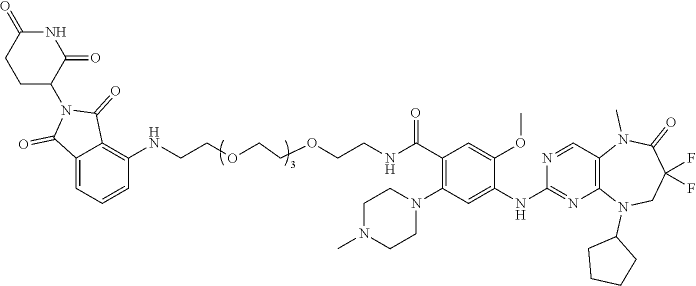 |
| 225 | 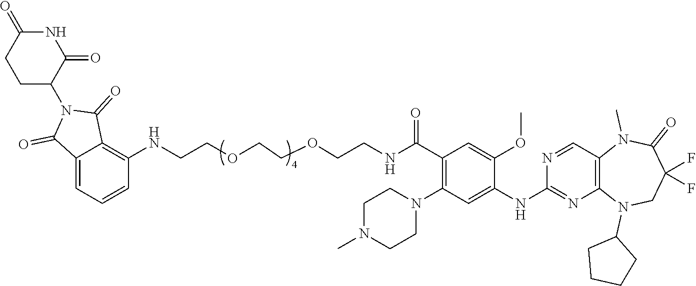 |

The compounds of the present invention were purified according to the following method and the structure was analyzed.

### Instruments

LCMS: Shimadzu LCMS-2020, Agilent 1200/G6110A, Agilent 1200/G1956A
HPLC: Agilent 1260 II LC, Agilent 1200/G6410B
NMR: BRUKER AVANCE III/400 MHz
SFC: SHIMADZU LC-30ADsf, Agilent 1260

### LCMS Analysis

LCMS data were recorded with Shimadzu LCMS-2020 or Agilent 1200/G6110A or Agilent 1200/G1956A equipped with an ESI (Electron Spray Ionization) device. 0.0375% TFA in water (solvent A) and 0.01875% TFA in ACN (solvent B) or 0.025% NH₃·H₂O in water (solvent A) and ACN (solvent B) were used as mobile phases. As a column, Kinetex EVO C18 (2.1×30 mm, 5 μm) or HALO C18 (3.0×30 mm, 2.7 μm) were used.

### HPLC Analysis

In HPLC analysis, Agilent 1260 II LC or Agilent 1200/G6410B were used. 0.0375% TFA in water (solvent A) and 0.01875% TFA in ACN (solvent B) were used as the mobile phase. As a column, Zobrax Eclipse Plus C18 (4.6×150 mm, 3.5 μm) or YMC ODS A (4.6×150 mm, 3 μm) were used.

### NMR Analysis

¹H NMR spectrum was recorded with Bruker AVANCE III 400 MHz/5 mm Probe (BBO).

### SFC Analysis

In SFC analysis, SHIMADZU LC-30ADsf or Agilent 1260 were used. CO2 (solvent A) and 0.05% DEA in IPA+ACN (solvent B) or CO₂ (solvent A) and 0.05% DEA in MeOH+ACN (solvent B) or 0.05% DEA in ACN (solvent A) and 0.05% DEA in EtOH (solvent B) were used as the mobile phase. As a column, Chiralpak AD-3 (50×4.6 mm, 3 μm) or Chiralpak AS-3 (50×4.6 mm, 3 μm) or Chiralpak OJ-3 (50×4.6 mm, 3 μm) or Chiralpak IA-3 (50×4.6 mm, 3 μm) or Chiralpak OD (50×4.6 mm, 3 μm) or Chiralpak IC-3 (50×4.6 mm, 3 μm) or (S,S)Whelk-O1 (100×4.6 mm, 3.5 μm) were used.

### Example 1. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2-(2,6-dioxopiperidin-3-yl)-1,3-dioxoisoindolin-4-yl)amino)ethyl)piperidin-4-yl)-2-fluoro-5-methoxybenzamide (Compound 1)

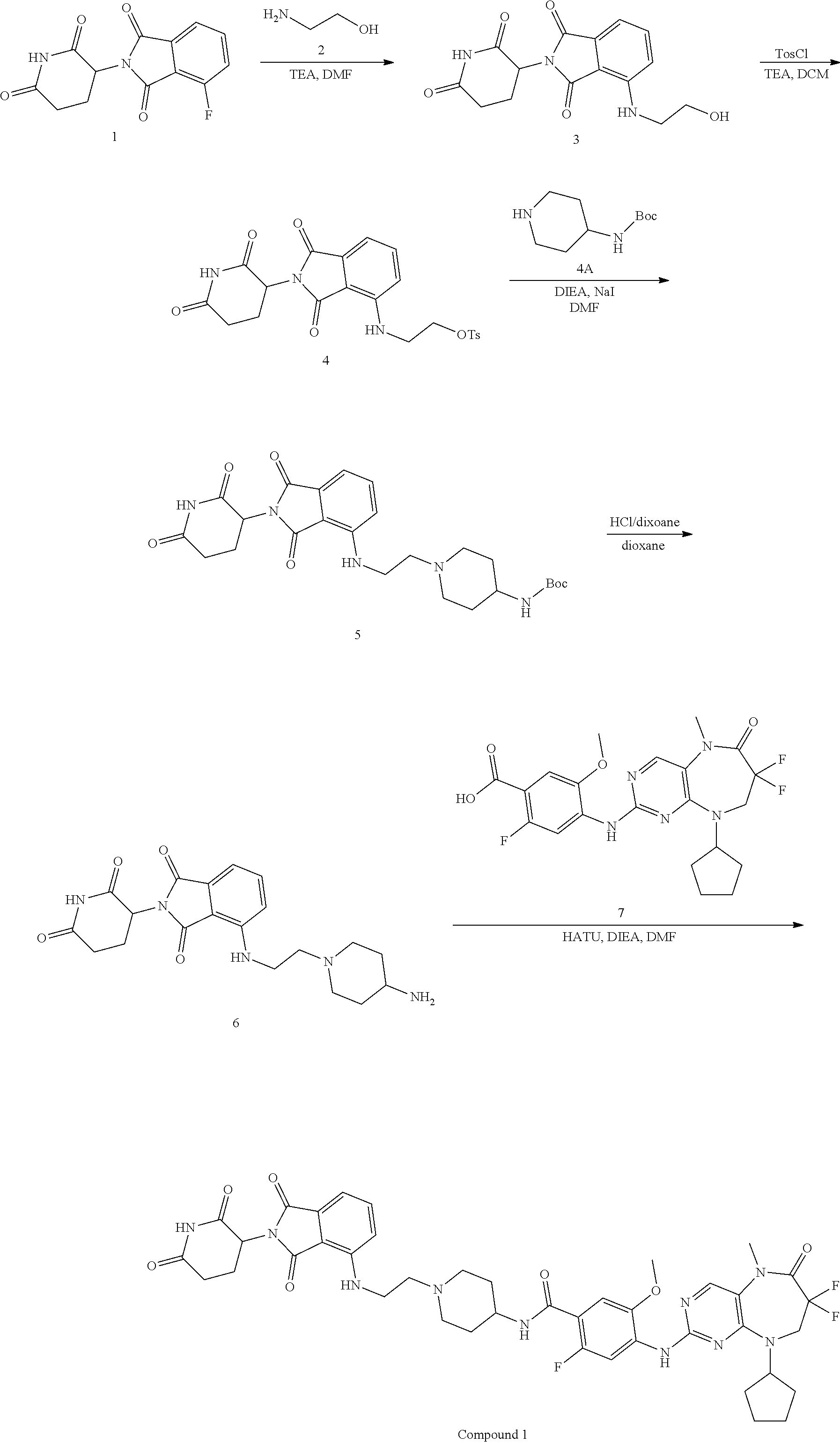

### Step 1. Synthesis of methyl 2-(2,6-dioxopiperidin-3-yl)-4-((2-hydroxyethyl)amino)isoindoline-1,3-dione (3)

A mixture of 2-(2,6-dioxopiperidin-3-yl)-4-fluoroisoindoline-1,3-dione (5 g, 18.10 mmol), 2-aminoethanol (1.44 g, 23.53 mmol, 1.42 mL) and TEA (4.58 g, 45.25 mmol, 6.30 mL) in DMF (35 mL) was stirred at 80° C. for 16 hours. LCMS showed the starting material was consumed completely and 48% of desired mass was detected. The mixture was concentrated in vacuo to afford 2-(2,6-dioxopiperidin-3-yl)-4-((2-hydroxyethyl)amino)isoindoline-1,3-dione (7.97 g) as a green oil, which was used for the next step directly. MS(M+H)⁺=317.9

### Step 2. Synthesis of 2-((2-(2,6-dioxopiperidin-3-yl)-1,3-dioxoisoindolin-4-yl)amino)ethyl 4-methylbenzenesulfonate (4)

To a solution of 2-(2,6-dioxopiperidin-3-yl)-4-((2-hydroxyethyl)amino)isoindoline-1,3-dione (7.97 g, 25.12 mmol) in DCM (40 mL) were added TEA (7.63 g, 75.36 mmol, 10.49 mL) and TosCl (9.58 g, 50.24 mmol), the mixture was stirred at 15° C. for 16 hours. LCMS showed 2-(2,6-dioxopiperidin-3-yl)-4-((2-hydroxyethyl)amino)isoindoline-1,3-dione was consumed completely and 30% of desired mass was detected. The reaction mixture was concentrated in vacuum to remove most of the solvent. The residue was diluted with H₂O (200 mL) and extracted with EtOAc (200 mL×3). The combined organic layers were washed with brine (300 mL×5), dried over Na₂SO₄, filtered and concentrated in vacuum. The residue was purified by flash silica gel chromatography (20 g SepaFlash® Silica Flash Column, Eluent of 12˜30%/Petroleum ether gradient @100 mL/min) to afford 2-((2-(2,6-dioxopiperidin-3-yl)-1,3-dioxoisoindolin-4-yl)amino)ethyl 4-methylbenzenesulfonate (860 mg, 1.82 mmol, 7.26% yield) as a green solid. MS(M+H)⁺=472.2

### Step 3. Synthesis of tert-butyl (1-(2-((2-(2,6-dioxopiperidin-3-yl)-1,3-dioxoisoindolin-4-yl)amino)ethyl)piperidin-4-yl)carbamate (5)

A mixture of 2-((2-(2,6-dioxopiperidin-3-yl)-1,3-dioxoisoindolin-4-yl)amino)ethyl 4-methylbenzenesulfonate (200 mg, 424.19 μmol), tert-butyl piperidin-4-ylcarbamate (101.95 mg, 509.03 μmol), DIEA (164.47 mg, 1.27 mmol, 221.66 μL) and NaI (6.36 mg, 42.42 μmol) in DMF (2 mL) was stirred at 80° C. for 5 hours. LCMS showed 2-((2-(2,6-dioxopiperidin-3-yl)-1,3-dioxoisoindolin-4-yl)amino)ethyl 4-methylbenzenesulfonate was consumed completely and 72% of desired mass was detected. The reaction mixture was diluted with H₂O (30 mL) and extracted with EtOAc (30 mL×3). The combined organic layers were washed with brine (50 mL×5), dried over Na₂SO₄, filtered and concentrated in vacuum. The residue was purified by flash silica gel chromatography (12 g SepaFlash® Silica Flash Column, Eluent of 25˜75% EtOAc/Petroleum ether gradient @100 mL/min) to afford tert-butyl (1-(2-((2-(2,6-dioxopiperidin-3-yl)-1,3-dioxoisoindolin-4-yl)amino)ethyl)piperidin-4-yl)carbamate (100 mg, 200.18 μmol, 47.19% yield) as a green solid. MS(M+H)⁺=500.2

### Step 4. Synthesis of 4-((2-(4-aminopiperidin-1-yl)ethyl)amino)-2-(2,6-dioxopiperidin-3-yl)isoindoline-1,3-dione (6)

To a solution of tert-butyl (1-(2-((2-(2,6-dioxopiperidin-3-yl)-1,3-dioxoisoindolin-4-yl)amino)ethyl)piperidin-4-yl)carbamate (100 mg, 200.18 μmol) in dioxane (5 mL) was added HCl/dioxane (4 M, 10 mL), the mixture was stirred at 15° C. for 2 hours. LCMS showed the starting material was consumed completely and 81% of desired mass was detected. The reaction mixture was concentrated in vacuo to afford 4-((2-(4-aminopiperidin-1-yl)ethyl)amino)-2-(2,6-dioxopiperidin-3-yl)isoindoline-1,3-dione (110 mg, HCl salt) as a green solid, which was used for the next step directly. MS(M+H)⁺=400.1

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2-(2,6-dioxopiperidin-3-yl)-1,3-dioxoisoindolin-4-yl)amino)ethyl)piperidin-4-yl)-2-fluoro-5-methoxybenzamide (Compound 1)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (90 mg, 193.37 μmol) in DMF (2 mL) were added DIEA (149.95 mg, 1.16 mmol, 202.09 μL) and HATU (110.29 mg, 290.06 μmol), the mixture was stirred at 15° C. for 15 minutes, to the mixture was added 4-((2-(4-aminopiperidin-1-yl)ethyl)amino)-2-(2,6-dioxopiperidin-3-yl)isoindoline-1,3-dione (109.58 mg, 251.38 μmol, HCl salt), the resulting mixture was stirred at 15° C. for 3 hour. LCMS showed 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid was consumed completely and 85% of desired mass was detected. To the mixture was added CH₃COOH to adjust pH<7. The resulting mixture was purified by prep-HPLC (column: Waters Xbridge 150*25 mm*5 μm; mobile phase: [water (10 mM NH₄HCO₃)-ACN]; B %: 48%-78%, min) followed by prep-HPLC (column: Phenomenex Synergi C₁₈ 150*25 mm*10 μm; mobile phase: [water (0.225% FA)-ACN]; B %: 16%-49%, 11 min), the eluent was freeze-dried to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2-(2,6-dioxopiperidin-3-yl)-1,3-dioxoisoindolin-4-yl)amino)ethyl)piperidin-4-yl)-2-fluoro-5-methoxybenzamide (42.8 mg, 49.53 μmol, 25.61% yield, 98% purity) as a yellow solid. MS(M+H)⁺=847.4

¹H NMR (400 MHz, DMSO-d₆) δ=11.09 (br s, 1H), 8.30 (s, 1H), 8.24 (d, J=13.3 Hz, 1H), 8.03 (s, 1H), 7.96 (dd, J=3.0, 7.5 Hz, 1H), 7.59 (dd, J=7.3, 8.4 Hz, 1H), 7.19 (d, J=6.7 Hz, 1H), 7.10 (d, J=8.7 Hz, 1H), 7.03 (d, J=7.0 Hz, 1H), 6.83-6.65 (m, 1H), 5.05 (dd, J=5.5, 12.9 Hz, 1H), 4.90-4.75 (m, 1H), 4.14-4.01 (m, 2H), 3.91 (s, 3H), 3.84-3.70 (m, 1H), 3.44-3.35 (m, 5H), 2.98-2.81 (m, 3H), 2.64-2.52 (m, 4H), 2.11 (t, J=10.7 Hz, 2H), 2.06-1.91 (m, 3H), 1.87-1.78 (m, 2H), 1.77-1.69 (m, 2H), 1.68-1.50 (m, 6H).

### Example 2. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2-(2,6-dioxopiperidin-3-yl)-1,3-dioxoisoindolin-4-yl)oxy)ethyl)piperidin-4-yl)-2-fluoro-5-methoxybenzamide (Compound 2)

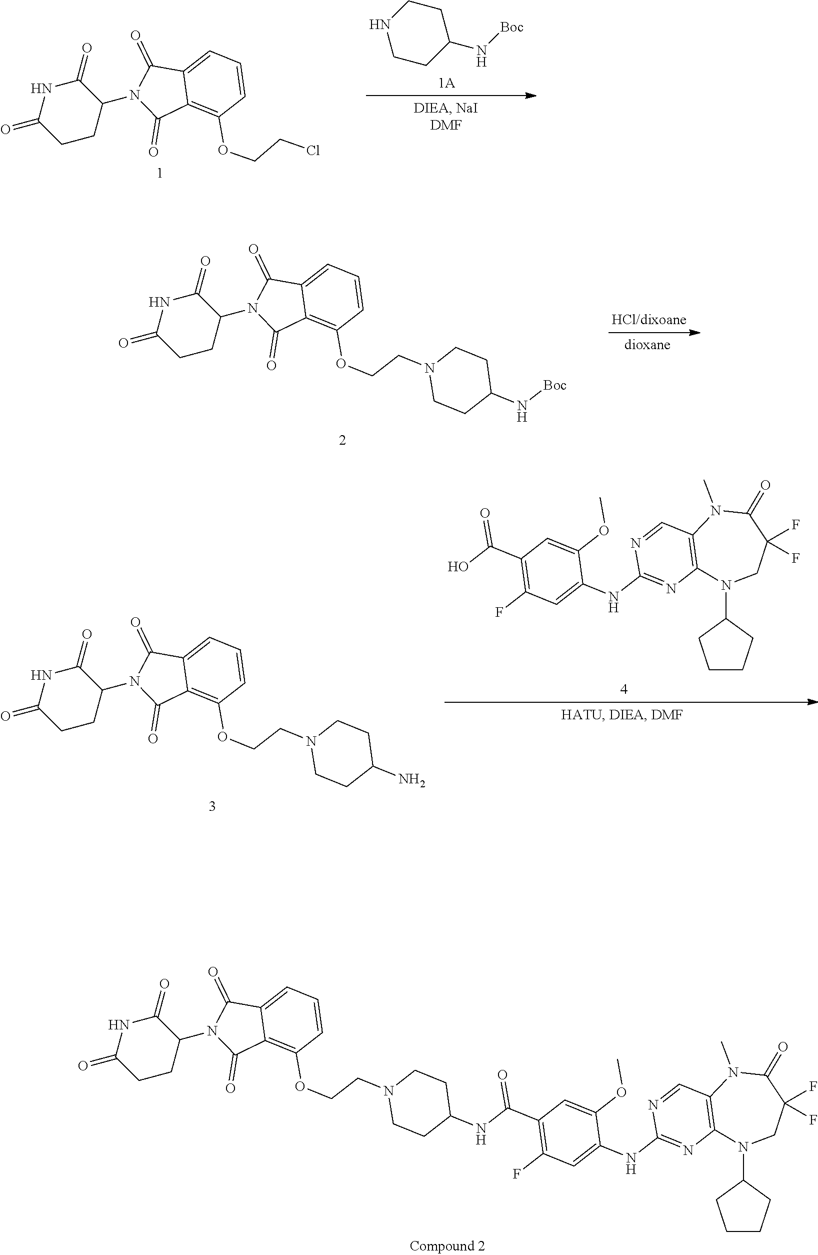 [SMILES: O=C1CCC(N2C(=O)c3cccc(OCCCl)c3C2=O)C(=O)N1 ; CC(C)(C)OC(=O)NC1CCNCC1 ; CC(C)(C)OC(=O)NC1CCN(CCOc2cccc3c2C(=O)N(C2CCC(=O)NC2=O)C3=O)CC1 ; NC1CCN(CCOc2cccc3c2C(=O)N(C2CCC(=O)NC2=O)C3=O)CC1 ; COc1cc(C(=O)O)c(F)cc1Nc1ncc2c(n1)N(C1CCCC1)CC(F)(F)C(=O)N2C ; COc1cc(C(=O)NC2CCN(CCOc3cccc4c3C(=O)N(C3CCC(=O)NC3=O)C4=O)CC2)c(F)cc1Nc1ncc2c(n1)N(C1CCCC1)CC(F)(F)C(=O)N2C]

The compound 2 was synthesized by the method described in the scheme similar to the method described in Example 1.

MS(M+H)⁺=848.7, ¹H NMR (400 MHz, DMSO-d₆) δ=11.10 (s, 1H), 8.30 (s, 1H), 8.24 (d, J=13.2 Hz, 1H), 8.03 (s, 1H), 7.89 (dd, J=2.9, 7.6 Hz, 1H), 7.85-7.78 (m, 1H), 7.55 (d, J=8.6 Hz, 1H), 7.45 (d, J=7.2 Hz, 1H), 7.19 (d, J=6.6 Hz, 1H), 5.17-5.04 (m, 1H), 4.88-4.76 (m, 1H), 4.39-4.26 (m, 2H), 4.07 (t, J=13.9 Hz, 2H), 3.91 (s, 3H), 3.80-3.70 (m, 1H), 3.44 (s, 3H), 2.98 (d, J=10.0 Hz, 2H), 2.93-2.82 (m, 1H), 2.82-2.70 (m, 2H), 2.64-2.53 (m, 2H), 2.22 (t, J=10.8 Hz, 2H), 2.08-1.88 (m, 3H), 1.87-1.68 (m, 4H), 1.68-1.47 (m, 6H).

### Example 3. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2-(2,6-dioxopiperidin-3-yl)-1,3-dioxoisoindolin-5-yl)oxy)ethyl)piperidin-4-yl)-2-fluoro-5-methoxybenzamide (Compound 3)

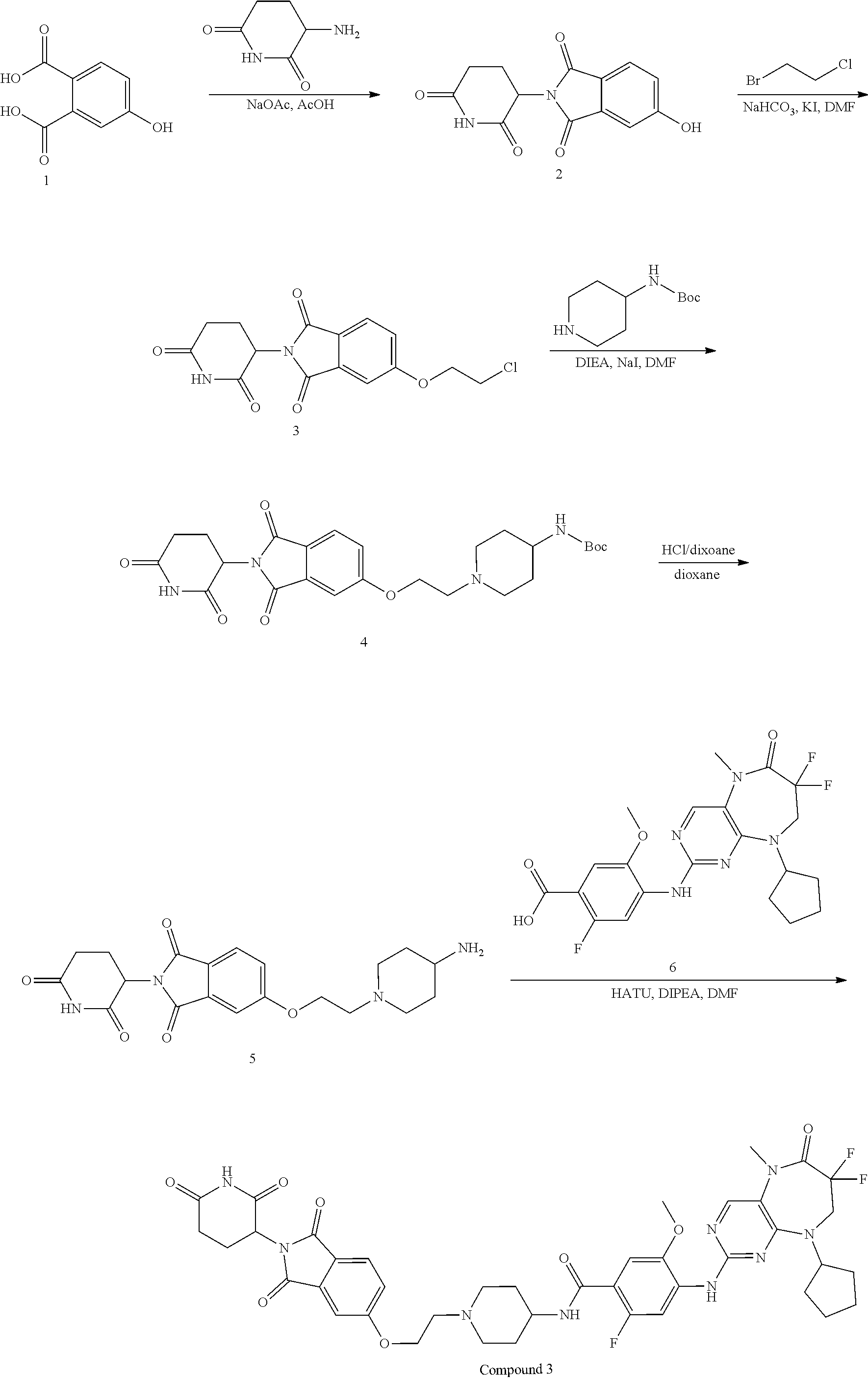

### Step 1. Synthesis of 2-(2,6-dioxopiperidin-3-yl)-5-hydroxyisoindoline-1,3-dione (2)

To a solution of 3-aminopiperidine-2,6-dione (13.56 g, 82.36 mmol, HCl salt) in CH₃COOH (100 mL) were added 4-hydroxyphthalic acid (10 g, 54.91 mmol) and NaOAc (13.51 g, 164.72 mmol), the mixture was stirred at 120° C. for 16 hr. LCMS showed desired mass, the mixture was filtered, the filter cake was washed with water (10 mL×3), MeOH (10 mL×3), then was collected and dried under vacuum to afford 2-(2,6-dioxopiperidin-3-yl)-5-hydroxyisoindoline-1,3-dione (11 g, crude) as a brown powder. MS(M+H)⁺=274.9

### Step 2. Synthesis of 5-(2-chloroethoxy)-2-(2,6-dioxopiperidin-3-yl)isoindoline-1,3-dione (3)

To a solution of 2-(2,6-dioxopiperidin-3-yl)-5-hydroxyisoindoline-1,3-dione (2 g, 7.29 mmol) in DMF (50 mL) were added NaHCO₃ (3.68 g, 43.76 mmol, 1.70 mL) and KI (242.14 mg, 1.46 mmol), then 1-bromo-2-chloroethane (3.14 g, 21.88 mmol, 1.81 mL) was added and the resulting mixture was stirred at 60° C. for 16 hr. LCMS showed desired mass, the mixture was diluted with water (20 mL), extracted with EtOAc (20 mL×3). The organic layer was dried over Na₂SO₄, filtered and concentrated under vacuum. The crude product was purified by Prep-HPLC (column: Waters Xbridge C18 150*50 mm*10 μm; mobile phase: [water(NH₄HCO₃)-ACN]; B %: 24%-54%, 11 min) and the eluent was lyophilized to afford 5-(2-chloroethoxy)-2-(2,6-dioxopiperidin-3-yl)isoindoline-1,3-dione (340 mg, 918.85 μmol, 12.60% yield, 91% purity) as a yellow powder. MS(M+H)⁺=336.9

### Step 3. Synthesis of tert-butyl (1-(2-((2-(2,6-dioxopiperidin-3-yl)-1,3-dioxoisoindolin-5-yl)oxy)ethyl)piperidin-4-yl)carbamate (4)

To a solution of 5-(2-chloroethoxy)-2-(2,6-dioxopiperidin-3-yl)isoindoline-1,3-dione (140 mg, 415.77 μmol) in DMF (8 mL) were added DIPEA (161.21 mg, 1.25 mmol, 217.26 μL) and NaI (12.46 mg, 83.15 μmol), then tert-butyl piperidin-4-ylcarbamate (166.54 mg, 831.54 μmol) was added, the mixture was stirred at 50° C. for 16 hr. LCMS showed desired mass, the mixture was diluted with water (6 mL), extracted with EtOAc (10 mL×3). The organic layer was dried over Na₂SO₄, filtered and concentrated under vacuum. The crude product was purified by Prep-HPLC (column: Phenomenex Synergi Polar-RP 100*25 mm*4 μm; water(TFA)-ACN; B %: 23%-43%, 7 min) and the eluent was lyophilized to afford tert-butyl (1-(2-((2-(2,6-dioxopiperidin-3-yl)-1,3-dioxoisoindolin-5-yl)oxy)ethyl)piperidin-4-yl)carbamate (50 mg, 94.90 μmol, 22.82% yield, 95% purity) as a red powder. MS(M+H)⁺=501.3

### Step 4. Synthesis of 5-(2-(4-aminopiperidin-1-yl)ethoxy)-2-(2,6-dioxopiperidin-3-yl)isoindoline-1,3-dione (5)

To a solution of tert-butyl (1-(2-((2-(2,6-dioxopiperidin-3-yl)-1,3-dioxoisoindolin-5-yl)oxy)ethyl)piperidin-4-yl)carbamate (50 mg, 99.89 μmol) in dioxane (4 mL) was added HCl/dioxane (4 M, 4 mL), the mixture was stirred at 25° C. for 1 h. LCMS showed desired mass, the mixture was concentrated under vacuum to afford 5-(2-(4-aminopiperidin-1-yl)ethoxy)-2-(2,6-dioxopiperidin-3-yl)isoindoline-1,3-dione (52 mg, crude, HCl salt) as a white powder. MS(M+H)⁺=400.9

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2-(2,6-dioxopiperidin-3-yl)-1,3-dioxoisoindolin-5-yl)oxy)ethyl)piperidin-4-yl)-2-fluoro-5-methoxybenzamide (Compound 3)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (85 mg, 182.63 μmol) in DMF (2 mL) were added HATU (104.16 mg, 273.94 μmol) and DIPEA (70.81 mg, 547.89 μmol, 95.43 μL), after stirring at 25° C. for 5 min, then 5-(2-(4-aminopiperidin-1-yl)ethoxy)-2-(2,6-dioxopiperidin-3-yl)isoindoline-1,3-dione (47.87 mg, 109.58 μmol, HCl salt) was added and the resulting mixture was stirred at 25° C. for 16 h. LCMS showed a major peak with desired mass. The mixture was diluted with water (3 mL), extracted with EtOAc (5 mL×3). The organic layer was dried over Na₂SO₄, filtered and concentrated under vacuum. The crude product was purified by Prep-HPLC (column: Phenomenex Synergi Polar-RP 100*25 mm*4 μm; mobile phase: [water(TFA)-ACN]; B %: 35%-55%, 7 min) and Prep-HPLC (column: Waters Xbridge 150*25 mm*5 μm; mobile phase: [water(NH₄HCO₃)-ACN]; B %: 43%-73%, 8 min), and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2-(2,6-dioxopiperidin-3-yl)-1,3-dioxoisoindolin-5-yl)oxy)ethyl)piperidin-4-yl)-2-fluoro-5-methoxybenzamide (38.2 mg, 41.18 μmol, 22.55% yield, 91.4% purity) as a white solid. MS(M+H)⁺=848.1

¹H NMR (400 MHz, DMSO-d₆) δ=11.13 (s, 1H), 8.32-8.21 (m, 2H), 8.05 (s, 1H), 7.92 (dd, J=7.64, 3.12 Hz, 1H), 7.84 (d, J=8.31 Hz, 1H), 7.47 (d, J=2.08 Hz, 1H), 7.38 (dd, J=8.31, 2.20 Hz, 1H), 7.19 (d, J=6.60 Hz, 1H), 5.13 (dd, J=12.96, 5.38 Hz, 1H), 4.82 (br t, J=8.01 Hz, 1H), 4.30 (t, J=5.50 Hz, 2H), 4.08 (t, J=13.82 Hz, 2H), 3.92 (s, 3H), 3.82-3.70 (m, 1H), 3.34 (s, 3H), 2.98-2.83 (m, 3H), 2.75 (t, J=5.44 Hz, 2H), 2.65-2.54 (m, 2H), 2.17 (t, J=10.76 Hz, 2H), 2.09-2.02 (m, 1H), 1.96 (d, J=2.81 Hz, 2H), 1.80 (d, J=10.76 Hz, 2H), 1.73 (s, 2H), 1.68-1.54 (m, 6H).

### Example 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-((2-(2,6-dioxopiperidin-3-yl)-1,3-dioxoisoindolin-4-yl)oxy)propyl)piperidin-4-yl)-2-fluoro-5-methoxybenzamide (Compound 4)

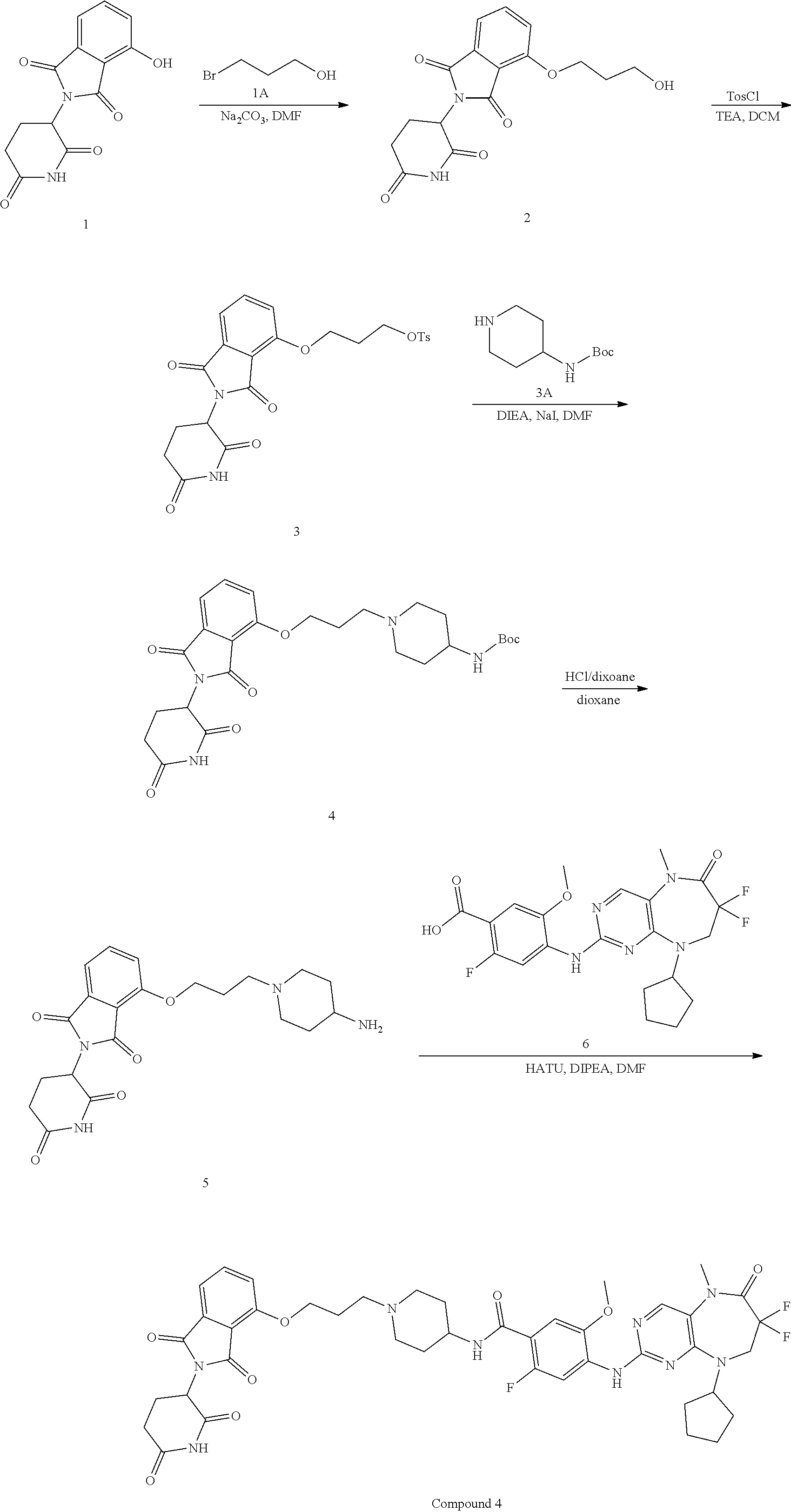

The compound 4 was synthesized by the method described in the scheme similar to the method described in Example 1.

MS(M+H)⁺=862.3, ¹H NMR (400 MHz, DMSO-d₆) δ=11.10 (s, 1H), 8.30 (s, 1H), 8.24 (d, J=13.4 Hz, 1H), 8.03 (s, 1H), 7.88 (dd, J=3.3, 7.6 Hz, 1H), 7.81 (dd, J=7.4, 8.4 Hz, 1H), 7.53 (d, J=8.6 Hz, 1H), 7.45 (d, J=7.3 Hz, 1H), 7.19 (d, J=6.6 Hz, 1H), 5.09 (dd, J=5.4, 12.8 Hz, 1H), 4.82 (quin, J=7.9 Hz, 1H), 4.25 (br t, J=6.1 Hz, 2H), 4.07 (br t, J=13.9 Hz, 2H), 3.91 (s, 3H), 3.81-3.69 (m, 1H), 3.27 (br s, 3H), 2.96-2.79 (m, 3H), 2.64-2.52 (m, 2H), 2.48-2.44 (m, 2H), 2.07-1.99 (m, 3H), 1.99-1.87 (m, 4H), 1.80 (br d, J=10.1 Hz, 2H), 1.73 (br s, 2H), 1.68-1.58 (m, 4H), 1.57-1.49 (m, 2H).

### Example 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2-(2,6-dioxopiperidin-3-yl)-1,3-dioxoisoindolin-4-yl)oxy)ethyl)piperidin-4-yl)-3-methoxybenzamide (Compound 5)

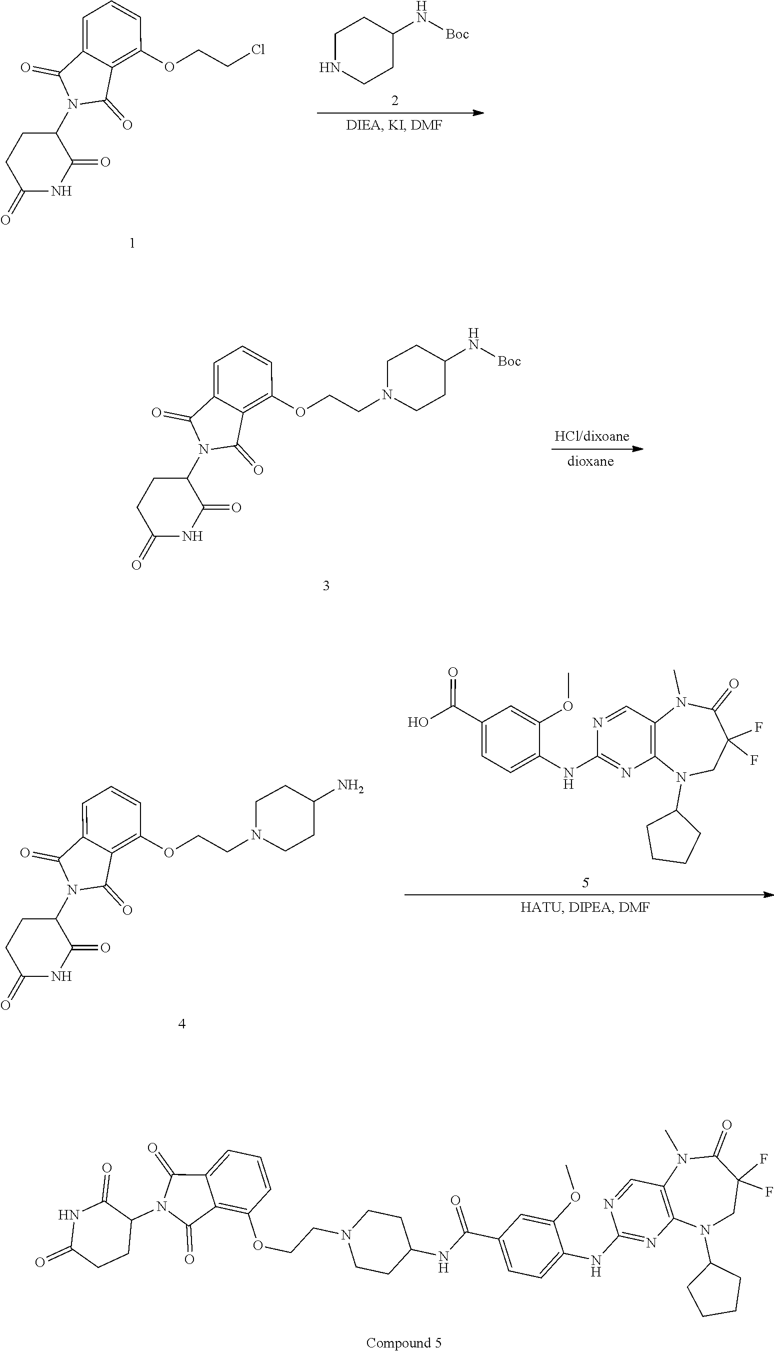 [SMILES: O=C1CCC(N2C(=O)c3cccc(OCCCl)c3C2=O)C(=O)N1 ; CC(C)(C)OC(=O)NC1CCNCC1 ; CC(C)(C)OC(=O)NC1CCN(CCOc2cccc3c2C(=O)N(C2CCC(=O)NC2=O)C3=O)CC1 ; NC1CCN(CCOc2cccc3c2C(=O)N(C2CCC(=O)NC2=O)C3=O)CC1 ; COc1cc(C(=O)O)ccc1Nc1ncc2c(n1)N(C1CCCC1)CC(F)(F)C(=O)N2C ; COc1cc(C(=O)NC2CCN(CCOc3cccc4c3C(=O)N(C3CCC(=O)NC3=O)C4=O)CC2)ccc1Nc1ncc2c(n1)N(C1CCCC1)CC(F)(F)C(=O)N2C]

The compound 5 was synthesized by the method described in the scheme similar to the method described in Example 1.

MS(M+H)⁺=830.0, ¹H NMR (400 MHz, DMSO-d₆) δ=11.11 (s, 1H), 8.29-8.25 (m, 2H), 8.08 (d, J=7.8 Hz, 1H), 7.97 (s, 1H), 7.86-7.80 (m, 1H), 7.57 (d, J=8.6 Hz, 1H), 7.51-7.44 (m, 3H), 5.10 (dd, J=5.4, 12.9 Hz, 1H), 4.82-4.71 (m, 1H), 4.38-4.31 (m, 2H), 4.07-4.02 (m, 2H), 3.94 (s, 3H), 3.82-3.72 (m, 1H), 3.32-3.28 (m, 3H), 3.06-3.02 (m, 2H), 2.95-2.83 (m, 1H), 2.81-2.78 (m, 2H), 2.64-2.57 (m, 2H), 2.26-2.19 (m, 2H), 2.07-2.00 (m, 1H), 1.98-1.89 (m, 2H), 1.83-1.76 (m, 2H), 1.74-1.68 (m, 2H), 1.65-1.56 (m, 6H).

### Example 6. Synthesis of 2-chloro-4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2-(2,6-dioxopiperidin-3-yl)-1,3-dioxoisoindolin-4-yl)oxy)ethyl)piperidin-4-yl)-5-methoxybenzamide (Compound 6)

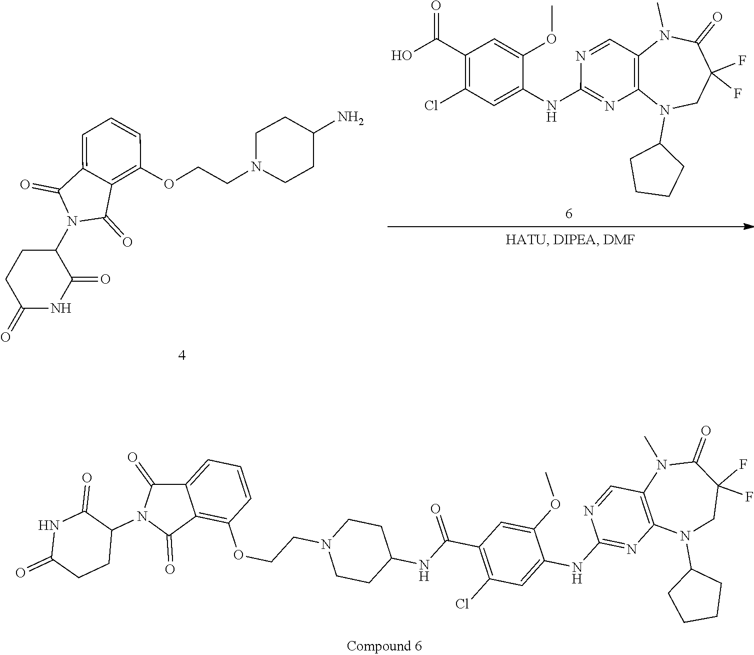 [SMILES: NC1CCN(CCOc2cccc3c2C(=O)N(C2CCC(=O)NC2=O)C3=O)CC1 ; COc1cc(C(=O)O)c(Cl)cc1Nc1ncc2c(n1)N(C1CCCC1)CC(F)(F)C(=O)N2C ; COc1cc(C(=O)NC2CCN(CCOc3cccc4c3C(=O)N(C3CCC(=O)NC3=O)C4=O)CC2)c(Cl)cc1Nc1ncc2c(n1)N(C1CCCC1)CC(F)(F)C(=O)N2C]

The compound 6 was synthesized by the method described in the scheme similar to the method described in Example 1.

MS(M+H)⁺=863.9, ¹H NMR (400 MHz, DMSO-d₆) δ=11.11 (s, 1H), 8.40 (s, 1H), 8.28 (s, 1H), 8.24 (d, J=7.8 Hz, 1H), 7.99 (s, 1H), 7.85-7.79 (m, 1H), 7.55 (d, J=8.6 Hz, 1H), 7.46 (d, J=7.2 Hz, 1H), 7.04 (s, 1H), 5.09 (dd, J=5.4, 12.7 Hz, 1H), 4.92-4.80 (m, 1H), 4.35-4.27 (m, 2H), 4.13-4.03 (m, 2H), 3.91 (s, 3H), 3.80-3.72 (m, 1H), 3.33 (s, 3H), 3.02-2.83 (m, 2H), 2.81-2.76 (m, 2H), 2.64-2.54 (m, 3H), 2.28-2.22 (m, 2H), 2.06-1.94 (m, 3H), 1.84-1.77 (m, 2H), 1.76-1.71 (m, 2H), 1.70-1.57 (m, 4H), 1.55-1.47 (m, 2H).

### Example 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((3R)-1-(2-((2-(2,6-dioxopiperidin-3-yl)-1,3-dioxoisoindolin-4-yl)oxy)ethyl)pyrrolidin-3-yl)-3-methoxybenzamide (Compound 7)

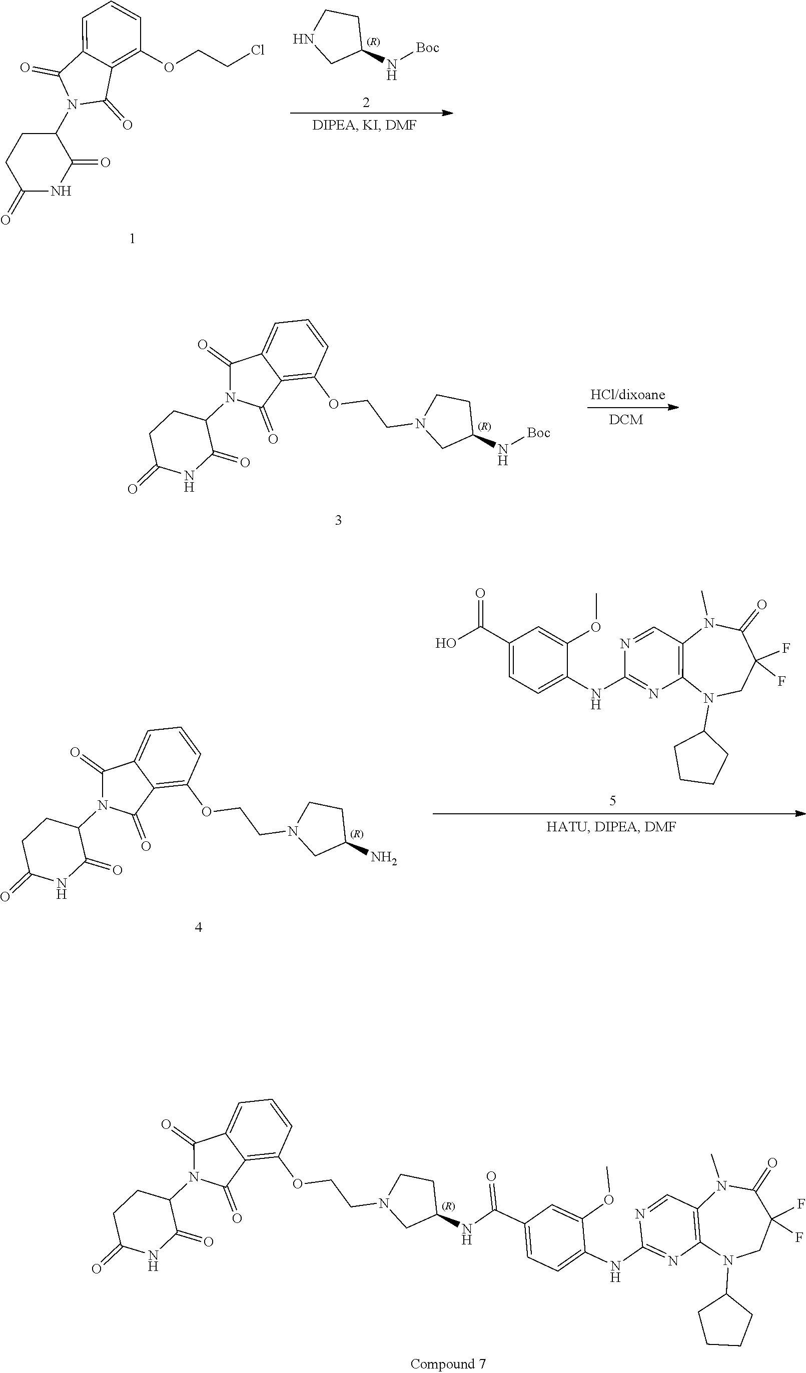

### Step 1. Synthesis of tert-butyl ((3R)-1-(2-((2-(2,6-dioxopiperidin-3-yl)-1,3-dioxoisoindolin-4-yl)oxy)ethyl)pyrrolidin-3-yl)carbamate (3)

To a solution of 4-(2-chloroethoxy)-2-(2,6-dioxopiperidin-3-yl)isoindoline-1,3-dione (500 mg, 1.48 mmol) and tert-butyl (R)-pyrrolidin-3-ylcarbamate (276.56 mg, 1.48 mmol) in DMF (7 mL) were added KI (246.49 mg, 1.48 mmol) and DIPEA (575.73 mg, 4.45 mmol, 775.92 μL). The mixture was stirred at 50° C. for 2 h. LCMS showed 4-(2-chloroethoxy)-2-(2,6-dioxopiperidin-3-yl)isoindoline-1,3-dione was consumed completely, and a peak (19%) with desired mass. The reaction mixture was diluted with water (100 mL) and extracted with EtOAc (30 mL×3). The combined organic layers were washed with brine (30 mL), dried over anhydrous sodium sulfate, filtered and concentrated under reduced pressure. The crude product was purified by reversed-phase HPLC (0.1% FA condition: Column: 120 g Flash Column Welch Ultimate XB_C₁₈ 20-40 μm; Flow rate: 85 mL/min; Mobile phase: MeCN/H₂O; Gradient B %: 5-30% 20 min; 30-100% 25 min, Instrument: TELEDYNE ISCO CombiFlashRf₁₅₀) and the eluent was lyophilized to afford tert-butyl ((3R)-1-(2-((2-(2,6-dioxopiperidin-3-yl)-1,3-dioxoisoindolin-4-yl)oxy)ethyl)pyrrolidin-3-yl)carbamate (120 mg, 239.25 μmol, 16.11% yield, 97% purity) as a white solid. MS(M+H)⁺=487.3

### Step 2. Synthesis of 4-(2-((R)-3-aminopyrrolidin-1-yl)ethoxy)-2-(2,6-dioxopiperidin-3-yl)isoindoline-1,3-dione (4)

A mixture of tert-butyl ((3R)-1-(2-((2-(2,6-dioxopiperidin-3-yl)-1,3-dioxoisoindolin-4-yl)oxy)ethyl)pyrrolidin-3-yl)carbamate (100 mg, 205.54 μmol), HCl/dioxane (4 M, 1.5 mL) in DCM (1.5 mL) was stirred at 25° C. for 0.5 h. LCMS showed tert-butyl ((3R)-1-(2-((2-(2,6-dioxopiperidin-3-yl)-1,3-dioxoisoindolin-4-yl)oxy)ethyl)pyrrolidin-3-yl)carbamate was consumed completely and a major peak (91%) with desired mass. The reaction mixture was concentrated under reduced pressure to afford 4-(2-((R)-3-aminopyrrolidin-1-yl)ethoxy)-2-(2,6-dioxopiperidin-3-yl)isoindoline-1,3-dione (100 mg, crude, HCl salt) as a white solid. MS(M+H)⁺=387.2

### Step 3. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((3R)-1-(2-((2-(2,6-dioxopiperidin-3-yl)-1,3-dioxoisoindolin-4-yl)oxy)ethyl)pyrrolidin-3-yl)-3-methoxybenzamide (Compound 7)

To a solution of 4-(2-((R)-3-aminopyrrolidin-1-yl)ethoxy)-2-(2,6-dioxopiperidin-3-yl)isoindoline-1,3-dione (100 mg, 236.48 μmol, HCl salt) and 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (105.81 mg, 236.48 μmol) in DMF (3 mL) were added HATU (134.88 mg, 354.73 μmol) and DIPEA (91.69 mg, 709.45 μmol, 123.57 μL). The mixture was stirred at 25° C. for 5 h. LCMS showed 4-(2-((R)-3-aminopyrrolidin-1-yl)ethoxy)-2-(2,6-dioxopiperidin-3-yl)isoindoline-1,3-dione was consumed completely, and a peak (77%) with desired mass. The reaction mixture was diluted with water (50 mL) and extracted with EtOAc (20 mL×3). The combined organic layers were washed with brine (20 mL), dried over anhydrous sodium sulfate, filtered and concentrated under reduced pressure. The residue was purified by column chromatography (SiO₂, MeOH/EtOAc=0/1 to 1/10). Then re-purified by prep-HPLC (neutral condition: column: Waters Xbridge 150×25 mm×5 μm; mobile phase: [water(NH₄HCO₃)-ACN]; B %: 40%-70%, 10 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-((3R)-1-(2-((2-(2,6-dioxopiperidin-3-yl)-1,3-dioxoisoindolin-4-yl)oxy)ethyl)pyrrolidin-3-yl)-3-methoxybenzamide (33.4 mg, 39.30 μmol, 16.62% yield, 96% purity) as a white solid. MS(M+H)⁺=816.0

¹H NMR (400 MHz, DMSO-d₆) δ=11.11 (s, 1H), 8.37 (d, J=7.1 Hz, 1H), 8.30-8.24 (m, 2H), 7.97 (s, 1H), 7.82 (dd, J=7.4, 8.5 Hz, 1H), 7.58-7.46 (m, 4H), 5.13-5.04 (m, 1H), 4.83-4.72 (m, 1H), 4.48-4.38 (m, 1H), 4.35-4.30 (m, 2H), 4.06-3.98 (m, 2H), 3.94 (s, 3H), 3.33 (s, 3H), 2.94-2.82 (m, 5H), 2.66-2.58 (m, 3H), 2.56-2.53 (m, 1H), 2.21-2.10 (m, 1H), 2.06-1.91 (m, 3H), 1.85-1.75 (m, 1H), 1.74-1.66 (m, 2H), 1.65-1.56 (m, 4H).

### Example 8. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((3R)-1-(3-((2-(2,6-dioxopiperidin-3-yl)-1,3-dioxoisoindolin-4-yl)oxy)propyl)pyrrolidin-3-yl)-3-methoxybenzamide (Compound 8)

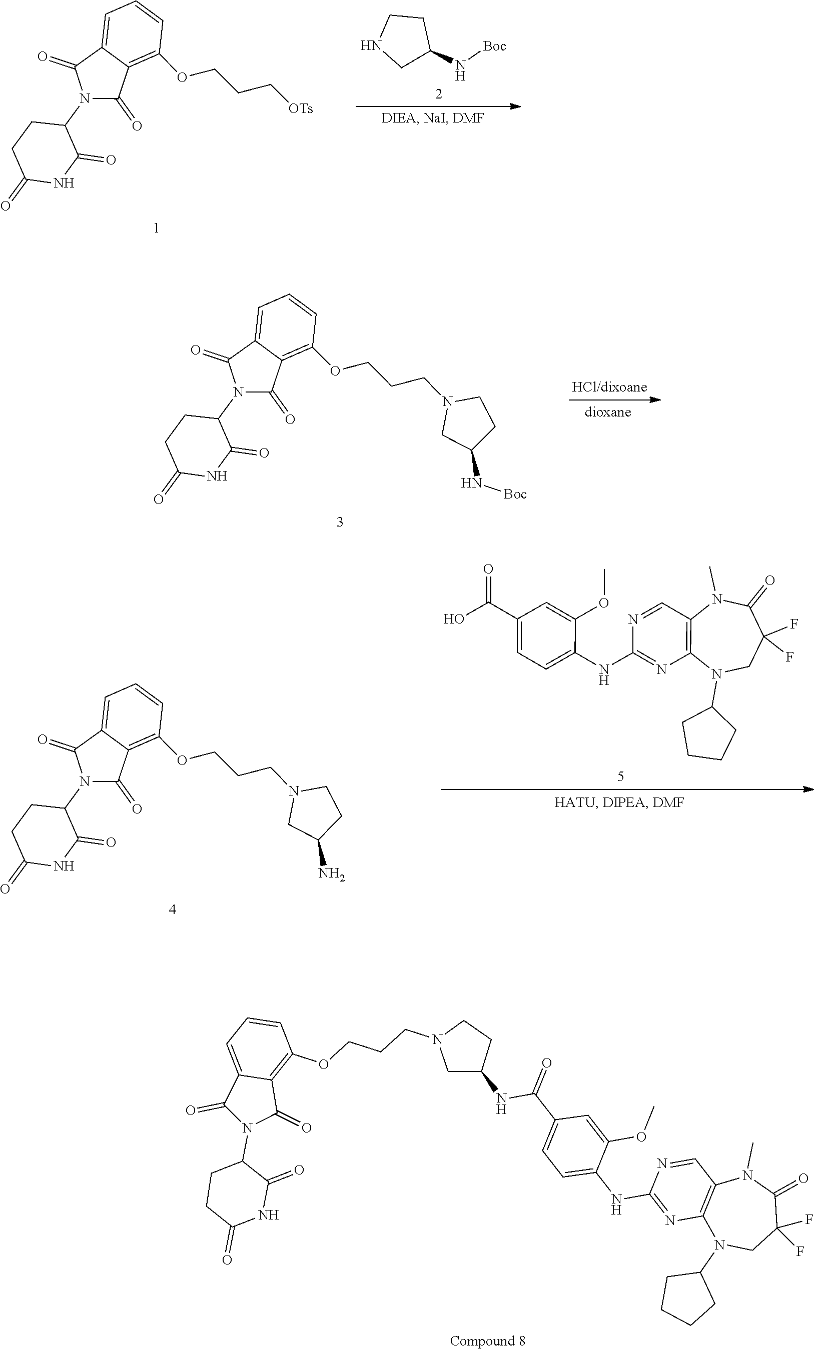 [SMILES: Cc1ccc(S(=O)(=O)OCCCOc2cccc3c2C(=O)N(C2CCC(=O)NC2=O)C3=O)cc1 ; CC(C)(C)OC(=O)N[C@@H]1CCNC1 ; CC(C)(C)OC(=O)N[C@@H]1CCN(CCCOc2cccc3c2C(=O)N(C2CCC(=O)NC2=O)C3=O)C1 ; N[C@@H]1CCN(CCCOc2cccc3c2C(=O)N(C2CCC(=O)NC2=O)C3=O)C1 ; COc1cc(C(=O)O)ccc1Nc1ncc2c(n1)N(C1CCCC1)CC(F)(F)C(=O)N2C ; COc1cc(C(=O)N[C@@H]2CCN(CCCOc3cccc4c3C(=O)N(C3CCC(=O)NC3=O)C4=O)C2)ccc1Nc1ncc2c(n1)N(C1CCCC1)CC(F)(F)C(=O)N2C]

The compound 8 was synthesized by the method described in the scheme similar to the method described in Example 7.

MS (M+H)⁺=830.3, ¹H NMR (400 MHz, DMSO-d₆) δ=11.11 (s, 1H), 8.47 (br d, J=3.6 Hz, 1H), 8.36-8.20 (m, 2H), 7.99 (s, 1H), 7.83 (dd, J=7.4, 8.4 Hz, 1H), 7.62-7.38 (m, 4H), 5.07 (dd, J=5.4, 12.6 Hz, 1H), 4.76 (quin, J=8.1 Hz, 1H), 4.54-4.41 (m, 1H), 4.30 (br t, J=5.9 Hz, 2H), 4.04 (br t, J=14.0 Hz, 2H), 3.92 (s, 3H), 3.32 (br s, 3H), 3.02-2.78 (m, 6H), 2.62-2.54 (m, 3H), 2.28-2.25 (m, 1H), 2.13-2.02 (m, 2H), 2.02-1.86 (m, 4H), 1.74-1.71 (m, 2H), 1.65-1.48 (m, 4H).

### Example 9. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((3S)-1-(2-((2-(2,6-dioxopiperidin-3-yl)-1,3-dioxoisoindolin-4-yl)oxy)ethyl)pyrrolidin-3-yl)-3-methoxybenzamide (Compound 9)

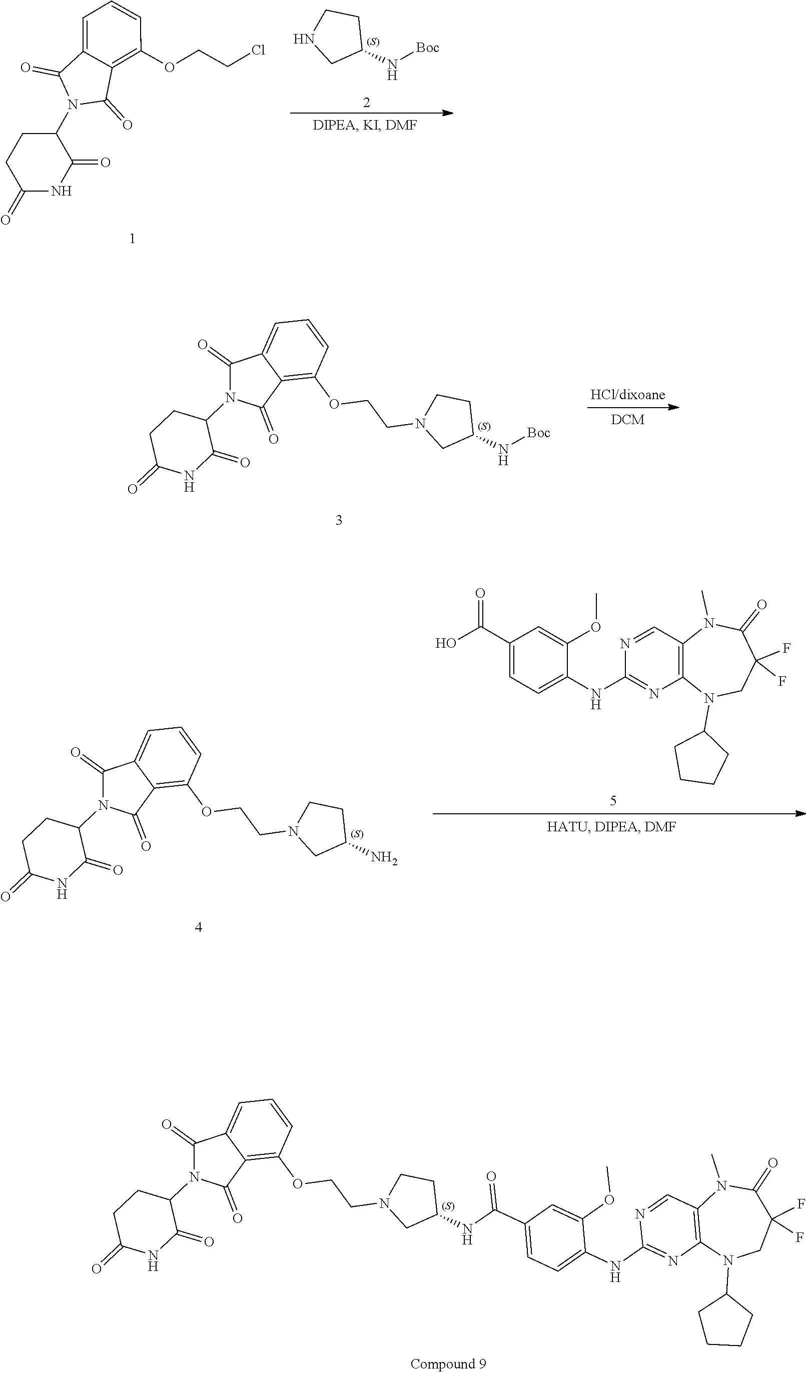

The compound 9 was synthesized by the method described in the scheme similar to the method described in Example 7.

MS(M+H)⁺=816.0, ¹H NMR (400 MHz, DMSO-d₆) δ=11.11 (s, 1H), 8.37 (d, J=7.2 Hz, 1H), 8.31-8.22 (m, 2H), 7.97 (s, 1H), 7.82 (dd, J=7.4, 8.5 Hz, 1H), 7.57-7.44 (m, 4H), 5.12-5.04 (m, 1H), 4.79-4.69 (m, 1H), 4.48-4.38 (m, 1H), 4.37-4.31 (m, 2H), 4.06-4.02 (m, 2H), 3.94 (s, 3H), 3.33 (s, 3H), 2.94-2.83 (m, 5H), 2.69-2.58 (m, 3H), 2.57-2.54 (m, 1H), 2.21-2.11 (m, 1H), 2.05-1.91 (m, 3H), 1.84-1.78 (m, 1H), 1.74-1.67 (m, 2H), 1.64-1.56 (m, 4H).

### Example 10. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((3S)-1-(3-((2-(2,6-dioxopiperidin-3-yl)-1,3-dioxoisoindolin-4-yl)oxy)propyl)pyrrolidin-3-yl)-3-methoxybenzamide (Compound 10)

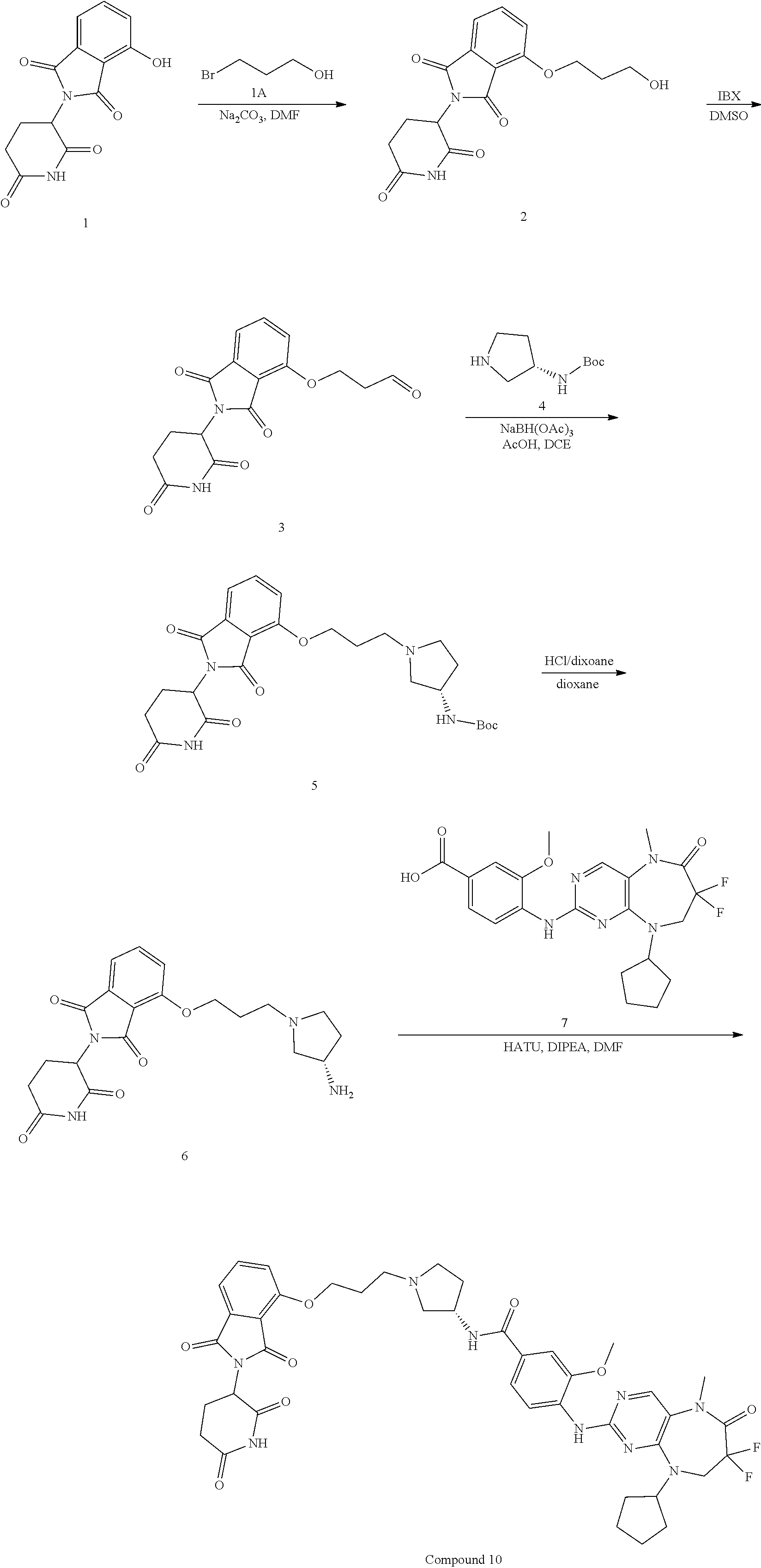

### Step 1. Synthesis of 2-(2,6-dioxopiperidin-3-yl)-4-(3-hydroxypropoxy)isoindoline-1,3-dione (2)

To a solution of 2-(2,6-dioxopiperidin-3-yl)-4-hydroxyisoindoline-1,3-dione (3 g, 10.94 mmol) and 3-bromopropan-1-ol (1.67 g, 12.03 mmol, 1.09 mL) in DMF (100 mL) was added Na₂CO₃ (5.80 g, 54.70 mmol), the mixture was stirred at 80° C. for 16 hours. LCMS showed 2-(2,6-dioxopiperidin-3-yl)-4-hydroxyisoindoline-1,3-dione was consumed completely and a peak (82%) with desired mass. The reaction mixture was diluted with H₂O (600 mL) and extracted with EtOAc (300 mL×4). The combined organic layers were washed with brine (500 mL×5), dried over Na₂SO₄, filtered and concentrated in vacuum. The residue was triturated with a mixture of MTBE (30 mL) and EtOAc (10 mL) for 10 minutes, the suspension was filtered and the filter cake was washed with MTBE (10 mL). The filter cake was collected and dried to afford 2-(2,6-dioxopiperidin-3-yl)-4-(3-hydroxypropoxy)isoindoline-1,3-dione (910 mg, 2.74 mmol, 25.03% yield) as a light brown solid. MS(M+H)⁺=333.0

### Step 2. Synthesis of 3-((2-(2,6-dioxopiperidin-3-yl)-1,3-dioxoisoindolin-4-yl)oxy)propanal (3)

To a solution of 2-(2,6-dioxopiperidin-3-yl)-4-(3-hydroxypropoxy)isoindoline-1,3-dione (410 mg, 1.23 mmol) in DMSO (3 mL) was added IBX (863.72 mg, 3.08 mmol), the mixture was stirred at 25° C. for 2 hours. LCMS showed trace of the starting material remained and a major peak (95%) of desired mass was detected. The reaction mixture was used directly. Compound (3-((2-(2,6-dioxopiperidin-3-yl-1,3-dioxoisoindolin-4-yl)oxy)propanal (410 mg)) was obtained as a brown liquid. MS(M+H)⁺=331.0

### Step 3. Synthesis of tert-butyl ((3S)-1-(3-((2-(2,6-dioxopiperidin-3-yl)-1,3-dioxoisoindolin-4-yl)oxy)propyl)pyrrolidin-3-yl)carbamate (5)

To a solution of tert-butyl (S)-pyrrolidin-3-ylcarbamate (346.80 mg, 1.86 mmol) and 3-((2-(2,6-dioxopiperidin-3-yl)-1,3-dioxoisoindolin-4-yl)oxy)propanal (410 mg, 1.24 mmol) in DCE (12 mL) was added AcOH (7.45 mg, 124.13 μmol, 7.10 μL), the mixture was stirred at 25° C. for 30 minutes, to the mixture was added NaBH(OAc)₃ (789.26 mg, 3.72 mmol), the resulting mixture was stirred at 25° C. for 12 hours. LCMS showed the trace of 3-((2-(2,6-dioxopiperidin-3-yl)-1,3-dioxoisoindolin-4-yl)oxy)propanal remained and 40% of desired mass was detected. The reaction mixture was filtered and the filter cake was washed with ACN (10 mL), the filtrate was concentrated in vacuum to afford the residue. The residue was purified by prep-HPLC (column: Waters Xbridge C18 150*50 mm*10 μm; mobile phase: [water(NH4HCO3)-ACN]; B %: 24%-54%, 11 min), the eluent was freeze-dried to afford tert-butyl ((3S)-1-(3-((2-(2,6-dioxopiperidin-3-yl)-1,3-dioxoisoindolin-4-yl)oxy)propyl)pyrrolidin-3-yl)carbamate (285 mg, 569.38 μmol, 45.87% yield) as a off-white solid. MS(M+H)⁺=501.2

### Step 4. Synthesis of 4-(3-((S)-3-aminopyrrolidin-1-yl)propoxy)-2-(2,6-dioxopiperidin-3-yl)isoindoline-1,3-dione (6)

To a solution of tert-butyl ((3S)-1-(3-((2-(2,6-dioxopiperidin-3-yl)-1,3-dioxoisoindolin-4-yl)oxy)propyl)pyrrolidin-3-yl)carbamate (150 mg, 299.67 μmol) in dioxane (1 mL) was added HCl/dioxane (4 M, 1 mL), the mixture was stirred at 25° C. for 1 hour. LCMS showed the starting material was consumed completely and 94% of desired mass was detected. The reaction mixture was concentrated in vacuum at 40° C. to afford 4-(3-((S)-3-aminopyrrolidin-1-yl)propoxy)-2-(2,6-dioxopiperidin-3-yl)isoindoline-1,3-dione (130 mg, HCl salt) as a brown solid, which was used directly. MS(M+H)⁺=401.1

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((3S)-1-(3-((2-(2,6-dioxopiperidin-3-yl)-1,3-dioxoisoindolin-4-yl)oxy)propyl)pyrrolidin-3-yl)-3-methoxybenzamide (Compound 10)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (80 mg, 178.80 μmol) in DMF (2 mL) were added HATU (101.98 mg, 268.20 μmol) and DIPEA (138.65 mg, 1.07 mmol, 186.86 μL), the mixture was stirred at 25° C. for 15 minutes, to the mixture was added 4-(3-((S)-3-aminopyrrolidin-1-yl)propoxy)-2-(2,6-dioxopiperidin-3-yl)isoindoline-1,3-dione (124.98 mg, 286.08 μmol, HCl salt), the mixture was stirred at 25° C. for 12 hours. LCMS showed 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid was consumed completely and 59% of desired mass was detected. To the mixture was added CH₃COOH to adjust pH<7. The mixture was stirred at prep-HPLC (column: Phenomenex C18 75*30 mm*3 μm; mobile phase: [water(FA)-ACN]; B %: 18%-48%, 7 min) followed by prep-HPLC (column: Waters Xbridge 150*25 mm*5 μm; mobile phase: [water(NH4HCO3)-ACN]; B %: 30%-60%, 8 min), the eluent was freeze-dried to afford 4-((9-cyclopentyl-7,7-difluoro-5-methyl-6-oxo-6,7,8,9-tetrahydro-5H-pyrimido[4,5-b][1,4]diazepin-2-yl)amino)-N-((3S)-1-(3-((2-(2,6-dioxopiperidin-3-yl)-1,3-dioxoisoindolin-4-yl)oxy)propyl)pyrrolidin-3-yl)-3-methoxybenzamide (29.3 mg, 34.25 μmol, 19.15% yield, 97% purity) as a off-white solid. MS(M+H)⁺=830.3

### Example 11. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2-(2,6-dioxopiperidin-3-yl)-1,3-dioxoisoindolin-4-yl)thio)ethyl)piperidin-4-yl)-2-fluoro-5-methoxybenzamide (Compound 11)

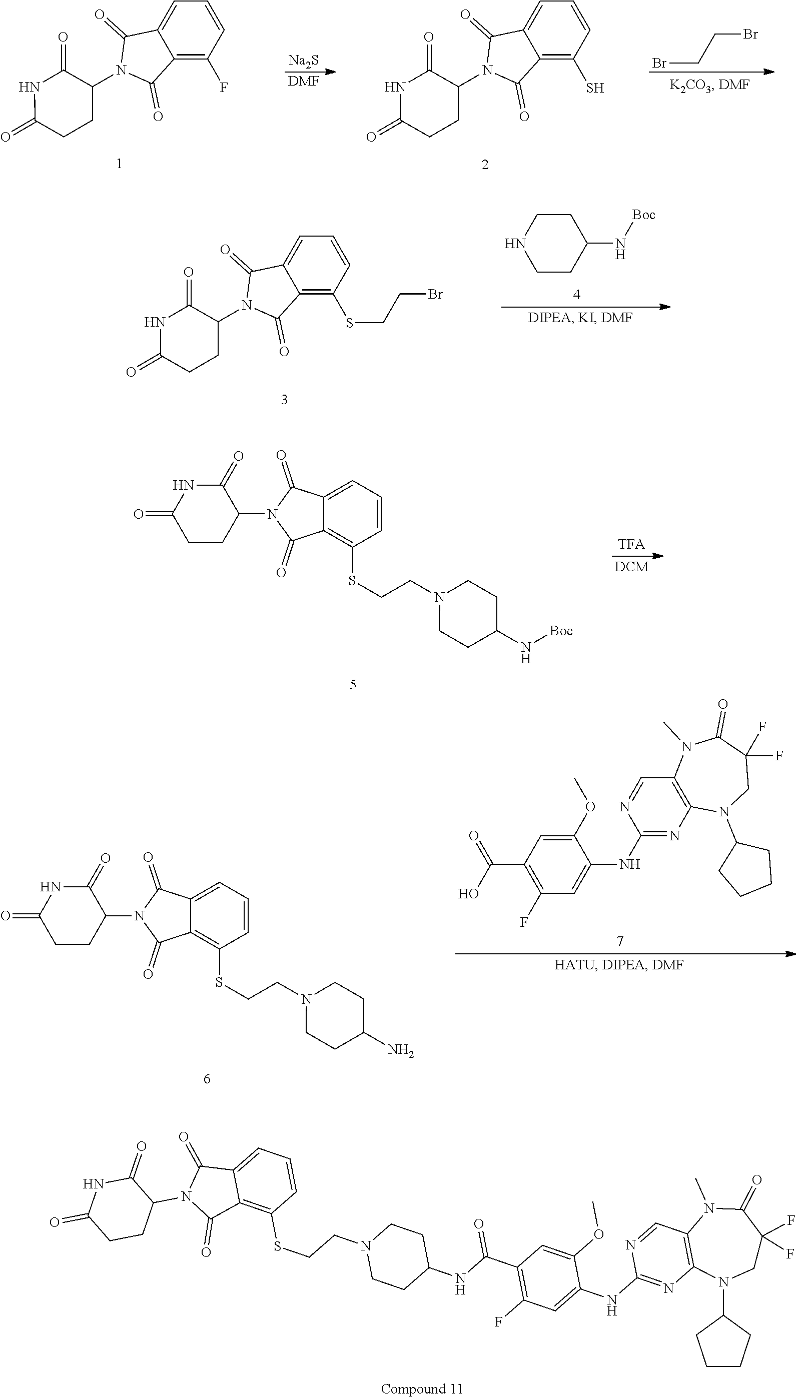

### Step 1. Synthesis of 2-(2,6-dioxopiperidin-3-yl)-4-mercaptoisoindoline-1,3-dione (2)

To a solution of 2-(2,6-dioxopiperidin-3-yl)-4-fluoroisoindoline-1,3-dione (5 g, 18.10 mmol) in DMF (50 mL) was added Na₂S (1.84 g, 23.53 mmol) in portions at 25° C. The resulting solution was stirred at 25° C. for 4 hrs. LCMS showed a main peak with desired mass. The reaction mixture was poured into ice-water (150 mL) and then 6N HCl solution was added to adjust pH to 3. The color of the mixture changed from blood red to light yellow and a lot of white solids were formed. The mixture was filtered. The filter cake was washed with water (30 mL×3), followed by acetone (30 mL×3). The resulting solid was collected and dried to afford 2-(2,6-dioxopiperidin-3-yl)-4-mercaptoisoindoline-1,3-dione (4.5 g, 15.50 mmol, 85.64% yield) as a gray solid.

### Step 2. Synthesis of 4-((2-bromoethyl)thio)-2-(2,6-dioxopiperidin-3-yl)isoindoline-1,3-dione (3)

To a mixture of 2-(2,6-dioxopiperidin-3-yl)-4-mercaptoisoindoline-1,3-dione (1.2 g, 4.13 mmol) and K₂CO₃ (1.14 g, 8.27 mmol) in DMF (15 mL) was added 1,2-dibromoethane (1.01 g, 5.37 mmol) at 25° C. The resulting mixture was stirred at 25° C. for 1 h. TLC (petroleum ether:EtOAc=1:1, R_{f}=0.75) showed the starting material was consumed completely. The reaction mixture was poured into brine (80 mL) and extracted with EtOAc (20 mL×3). The combined organic layers were washed with brine (15 mL×3), dried over Na₂SO₄ and concentrated to afford the crude product, which was purified by flash silica gel chromatography (10 g silica gel column, EtOAc/petroleum ether=10-40%, 60 mL/min) to afford 4-((2-bromoethyl)thio)-2-(2,6-dioxopiperidin-3-yl)isoindoline-1,3-dione (150 mg, 283.20 μmol, 6.85% yield, 75% purity) as a yellow solid. MS(M+H)⁺=399.0

¹H NMR (400 MHz, DMSO-d₆) δ=11.12 (s, 1H), 7.81-7.78 (m, 2H), 7.68-7.66 (m, 1H), 5.14-5.09 (m, 1H), 3.75-3.70 (m, 2H), 3.67-3.63 (m, 2H), 2.64-2.53 (m, 2H), 2.52-2.51 (m, 1H), 2.33-2.31 (m, 1H).

### Step 3. Synthesis of tert-butyl (1-(2-((2-(2,6-dioxopiperidin-3-yl)-1,3-dioxoisoindolin-4-yl)thio) ethyl)piperidin-4-yl)carbamate (5)

To a solution of 4-((2-bromoethyl)thio)-2-(2,6-dioxopiperidin-3-yl)isoindoline-1,3-dione (150 mg, 377.60 μmol) and tert-butyl N-(4-piperidyl) carbamate (113.44 mg, 566.40 μmol) in DMF (3 mL) were added DIPEA (146.41 mg, 1.13 mmol) and KI (6.27 mg, 37.76 μmol) at 25° C. The resulting mixture was stirred at 70° C. for 14 hrs. LCMS showed the starting material was consumed completely and the desired mass. The reaction mixture was poured into brine (20 mL) and extracted with EtOAc (8 mL×4). The combined organic layers were washed with brine (5 mL×2), dried over Na₂SO₄ and concentrated to afford the crude product, which was purified by flash silica gel chromatography (4 g silica gel column, EtOAc/petroleum ether=20-70%, 50 mL/min) to afford tert-butyl (1-(2-((2-(2,6-dioxopiperidin-3-yl)-1,3-dioxoisoindolin-4-yl)thio)ethyl)piperidin-4-yl)carbamate (120 mg, 213.70 μmol, 56.59% yield, 92% purity) as yellow oil. MS(M+H)⁺=517.2

### Step 4. Synthesis of 4-((2-(4-aminopiperidin-1-yl)ethyl)thio)-2-(2,6-dioxopiperidin-3-yl) isoindoline-1,3-dione (6)

A solution of tert-butyl (1-(2-((2-(2,6-dioxopiperidin-3-yl)-1,3-dioxoisoindolin-4-yl) thio)ethyl)piperidin-4-yl)carbamate (120 mg, 232.28 μmol) and TFA (924.00 mg, 8.10 mmol) in DCM (2 mL) was stirred at 25° C. for 30 mins. TLC (petroleum ether:EtOAc=0:1, R_{f}=0) showed the starting material was consumed completely. The reaction solution was concentrated to afford 4-((2-(4-aminopiperidin-1-yl)ethyl)thio)-2-(2,6-dioxopiperidin-3-yl)isoindoline-1,3-dione (120 mg, crude, TFA) was obtained as yellow oil. MS(M+H)⁺=417.1

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2-(2,6-dioxopiperidin-3-yl)-1,3-dioxoisoindolin-4-yl)thio)ethyl)piperidin-4-yl)-2-fluoro-5-methoxybenzamide (Compound 11)

To a mixture of 4-((2-(4-aminopiperidin-1-yl)ethyl)thio)-2-(2,6-dioxopiperidin-3-yl) isoindoline-1,3-dione (100 mg, 188.50 μmol, TFA), 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (78.96 mg, 169.65 μmol) and DIPEA (146.17 mg, 1.13 mmol) in DMF (2 mL) was added HATU (93.17 mg, 245.04 μmol) at 25° C. The resulting mixture was stirred at 25° C. for 14 hrs. LCMS showed a main peak with desired mass. The reaction solution was poured into brine (12 mL) and extracted with EtOAc (5 mL×4). The combined organic layers were dried over Na₂SO₄ and concentrated to afford the crude product. The crude product was purified by flash silica gel chromatography (4 g silica gel column, EtOAc/petroleum ether=20-100% and then MeOH/EtOAc=20%, 40 mL/min), followed by prep-HPLC (column: Waters Xbridge 150*25 mm*5 μm; mobile phase: [water(NH₄HCO₃)-ACN]; B %: 48%-78%, 10 min) and lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2-(2,6-dioxopiperidin-3-yl)-1,3-dioxoisoindolin-4-yl)thio)ethyl)piperidin-4-yl)-2-fluoro-5-methoxybenzamide (34 mg, 38.27 μmol, 20.30% yield, 97.23% purity) as a white solid. MS(M+H)⁺=864.0

¹H NMR (400 MHz, DMSO-d₆) δ=11.13 (s, 1H), 8.29 (s, 1H), 8.24 (d, J=14.4 Hz, 1H), 8.04 (s, 1H), 7.94-7.91 (m, 1H), 7.81-7.75 (m, 2H), 7.62 (d, J=8 Hz, 1H), 7.19 (d, J=8 Hz, 1H), 5.13-5.09 (m, 1H), 4.84-4.80 (m, 1H), 4.07 (t, J=16 Hz, 2H), 3.91 (s, 3H), 3.80-3.71 (m, 1H), 3.33 (s, 3H), 3.30-3.26 (m, 3H), 2.94-2.88 (m, 2H), 2.88-2.84 (m, 1H), 2.67-2.64 (m, 2H), 2.61-2.56 (m, 3H), 2.13-2.03 (m, 3H), 2.03-1.95 (m, 2H), 1.80-1.78 (m, 2H), 1.76-1.70 (m, 2H), 1.60-1.53 (m, 4H).

### Example 12. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-((2-(2,6-dioxopiperidin-3-yl)-1,3-dioxoisoindolin-4-yl)methyl)piperidin-4-yl)-3-methoxybenzamide (Compound 12)

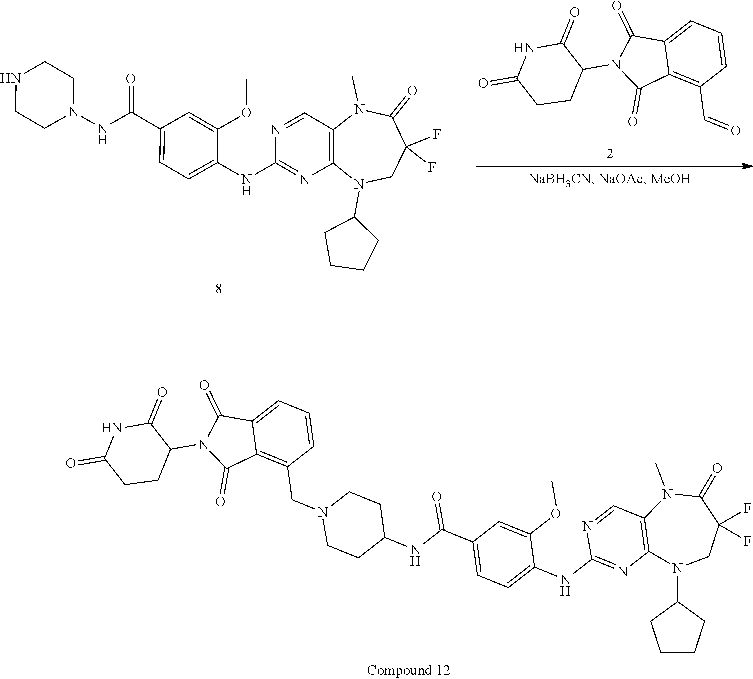 [SMILES: COc1cc(C(=O)NN2CCNCC2)ccc1Nc1ncc2c(n1)N(C1CCCC1)CC(F)(F)C(=O)N2C ; O=Cc1cccc2c1C(=O)N(C1CCC(=O)NC1=O)C2=O ; COc1cc(C(=O)NC2CCN(Cc3cccc4c3C(=O)N(C3CCC(=O)NC3=O)C4=O)CC2)ccc1Nc1ncc2c(n1)N(C1CCCC1)CC(F)(F)C(=O)N2C]

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(piperidin-4-yl)benzamide (40 mg, 70.67 μmol, HCl) in MeOH (1 mL) were added NaOAc (5.80 mg, 70.67 μmol) and 2-(2,6-dioxopiperidin-3-yl)-1,3-dioxoisoindoline-4-carbaldehyde (60 mg, 209.61 μmol) and the mixture was stirred at 20° C. for 2 h. NaBH₃CN (13.32 mg, 212.00 μmol) was added and the mixture was stirred at 20° C. for 3 h. LCMS showed 35% of desired mass. The mixture was concentrated under reduced pressure. The residue was purified by prep-HPLC (column: Phenomenex C18 75*30 mm*3 μm; mobile phase: [water (FA)-ACN]; B %: 12%-42%, 7 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-((2-(2,6-dioxopiperidin-3-yl)-1,3-dioxoisoindolin-4-yl) methyl)piperidin-4-yl)-3-methoxybenzamide (6.3 mg, 7.17 μmol, 10.14% yield, 91% purity) as white solid. MS(M+H)⁺=800.1

¹H NMR (400 MHz, DMSO-d₆) δ=11.20-11.09 (m, 1H), 8.33-8.23 (m, 2H), 8.17-8.08 (m, 1H), 8.01-7.79 (m, 4H), 7.52-7.39 (m, 2H), 5.26-5.06 (m, 1H), 4.82-4.70 (m, 1H), 4.09-4.00 (m, 2H), 3.99-3.95 (m, 1H), 3.95-3.91 (m, 3H), 3.88-3.75 (m, 1H), 3.32-3.30 (m, 3H), 2.97-2.84 (m, 2H), 2.65-2.57 (m, 1H), 2.45-2.35 (m, 3H), 2.25-2.12 (m, 2H), 2.11-2.02 (m, 1H), 1.99-1.88 (m, 2H), 1.86-1.75 (m, 1H), 1.74-1.52 (m, 9H).

### Example 13. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(2-(2,6-dioxopiperidin-3-yl)-1,3-dioxoisoindolin-4-yl)ethyl)piperidin-4-yl)-3-methoxybenzamide (Compound 13)

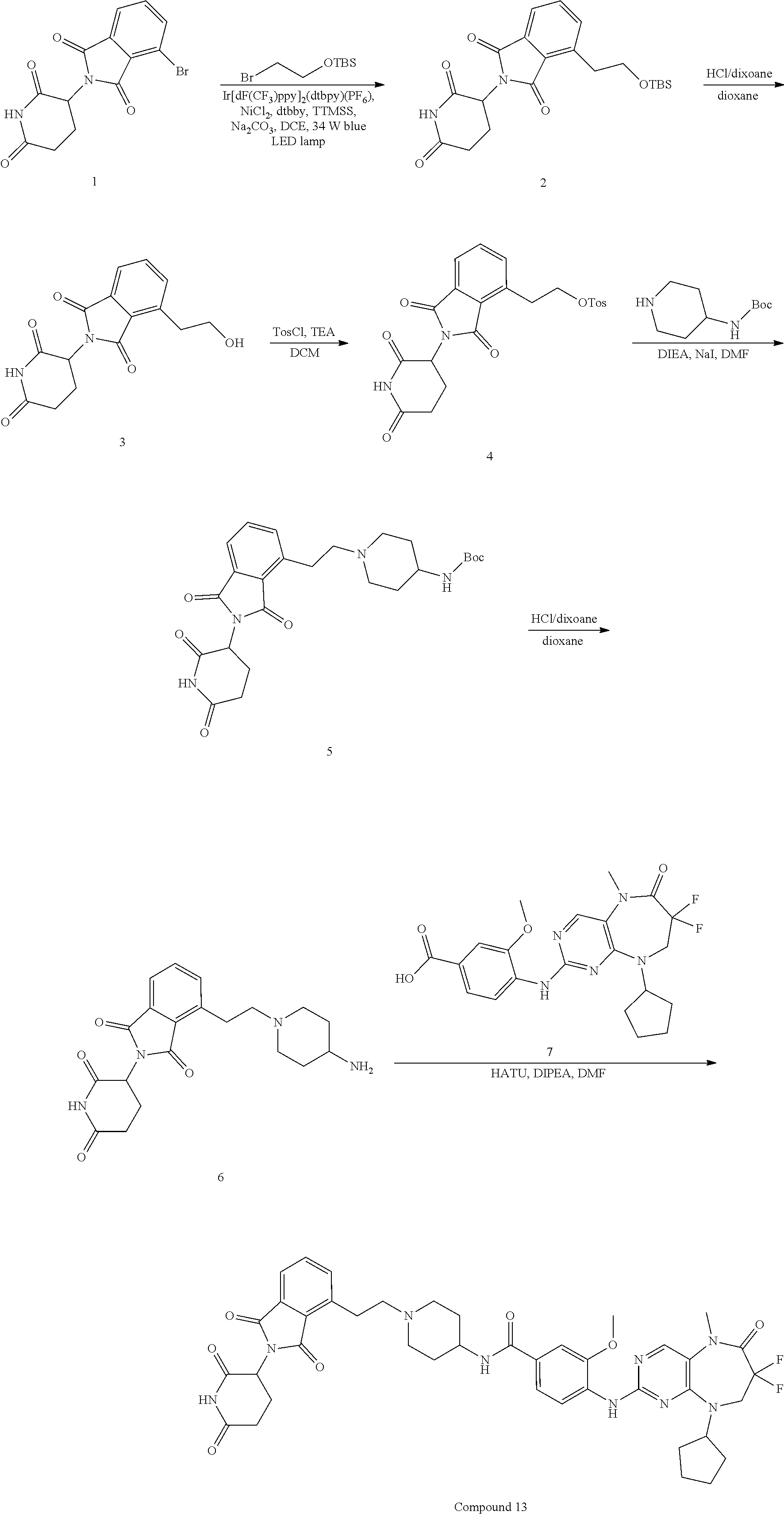

### Step 1. Synthesis of 4-(2-((tert-butyldimethylsilyl)oxy)ethyl)-2-(2,6-dioxopiperidin-3-yl)isoindoline-1,3-dione (2)

To an 100 mL vial equipped with a stir bar were added 4-bromo-2-(2,6-dioxopiperidin-3-yl)isoindoline-1,3-dione (1 g, 2.97 mmol), (2-bromoethoxy)(tert-butyl)dimethylsilane (922.48 mg, 3.86 mmol), Dtbbpy (5.90 mg, 14.83 μmol), TTMSS (737.59 mg, 2.97 mmol, 915.13 μL), Na₂CO₃ (628.78 mg, 5.93 mmol) in DCE (10 mL). The vial was sealed and placed under nitrogen. The reaction was stirred and irradiated with a 34 W blue LED lamp (7 cm away), with cooling fan to keep the reaction temperature at 25° C. for 14 hr. LCMS showed a peak (66%) with desired mass. The reaction mixture was diluted with brine (20 mL), extracted with EtOAc (20 mL×3). The combined organic layers were washed with brine (20 mL×2), dried over Na₂SO₄, filtered. The filtrate was concentrated under reduced pressure to give a residue. The residue was purified by flash silica gel chromatography (Biotage; 20 g SepaFlash® Silica Flash Column, Eluent of 20˜70% EtOAc/Petroleum ether gradient @40 mL/min) to afford 4-(2-((tert-butyldimethylsilyl)oxy)ethyl)-2-(2,6-dioxopiperidin-3-yl)isoindoline-1,3-di one (1.1 g, 2.27 mmol, 76.56% yield, 86% purity) as a yellow solid. MS(M+H)⁺=417.1

### Step 2. Synthesis of 2-(2,6-dioxopiperidin-3-yl)-4-(2-hydroxyethyl)isoindoline-1,3-dione (3)

To a solution of 4-(2-((tert-butyldimethylsilyl)oxy)ethyl)-2-(2,6-dioxopiperidin-3-yl)isoindoline-1,3-di one (650 mg, 1.56 mmol) in dioxane (25 mL) was added HCl/dioxane (4 M, 2.50 mL), the mixture was stirred at 0° C. for 0.5 h. LCMS showed a main peak with desired mass. The reaction mixture was diluted with H₂O (20 mL) and extracted with EtOAc (20 mL×3). The combined organic layers were washed with brine (50 mL×2), dried over Na₂SO₄, filtered. The filtrate was concentrated under reduced pressure to afford 2-(2,6-dioxopiperidin-3-yl)-4-(2-hydroxyethyl)isoindoline-1,3-dione (460 mg, crude) as a white solid. MS(M+H)⁺=303.3

### Step 3. Synthesis of 2-(2-(2,6-dioxopiperidin-3-yl)-1,3-dioxoisoindolin-4-yl)ethyl 4-methylbenzenesulfonate (4)

To a solution of 2-(2,6-dioxopiperidin-3-yl)-4-(2-hydroxyethyl)isoindoline-1,3-dione (500 mg, 1.65 mmol) in DCM (5 mL) were added TosCl (378.42 mg, 1.98 mmol), TEA (502.13 mg, 4.96 mmol, 690.68 μL), the mixture was stirred at 25° C. for 16 h. LCMS showed a peak (50%) with desired mass. The reaction mixture was diluted with H₂O (20 mL). The organic phase was separated, the aqueous phase was extracted with EtOAc (10 mL×2). The combined organic layers were washed with brine (20 mL×2), dried over Na₂SO₄, filtered. The filtrate was concentrated under reduced pressure to give a residue. The residue was purified by flash silica gel chromatography (Biotage; 4 g SepaFlash® Silica Flash Column, Eluent of 15˜50% EtOAc/Petroleum ether gradient @40 mL) to afford 2-(2-(2,6-dioxopiperidin-3-yl)-1,3-dioxoisoindolin-4-yl)ethyl 4-methylbenzenesulfonate (450 mg, 916.82 μmol, 55.43% yield, 93% purity) as a yellow oil. MS(M+H)⁺=456.9

### Step 4. Synthesis of tert-butyl (1-(2-(2-(2,6-dioxopiperidin-3-yl)-1,3-dioxoisoindolin-4-yl)ethyl)piperidin-4-yl)carbamate (5)

To a solution of 2-(2-(2,6-dioxopiperidin-3-yl)-1,3-dioxoisoindolin-4-yl)ethyl 4-methylbenzenesulfonate (440 mg, 963.92 μmol) in DMF (5 mL) were added tert-butyl N-(4-piperidyl)carbamate (212.36 mg, 1.06 mmol), DIPEA (124.58 mg, 963.92 μmol, 167.90 μL) and NaI (14.45 mg, 96.39 μmol), the mixture was stirred at 60° C. for 16 h. LCMS showed a peak (46%) with desired mass. The reaction mixture was diluted with H₂O (20 mL), extracted with EtOAc (10 mL×2). The combined organic layers were washed with brine (20 mL×2), dried over Na₂SO₄, filtered, and the filtrate was concentrated under reduced pressure to give a residue. The residue was purified by flash silica gel chromatography (Biotage; 12 g SepaFlash® Silica Flash Column, Eluent of 20˜70% EtOAc/Petroleum ether gradient @40 mL/min) to afford tert-butyl (1-(2-(2-(2,6-dioxopiperidin-3-yl)-1,3-dioxoisoindolin-4-yl)ethyl)piperidin-4-yl)carbamate (220 mg, 404.09 μmol, 41.92% yield, 89% purity) as a yellow oil. MS(M+H)⁺=485.2

### Step 5. Synthesis of 4-(2-(4-aminopiperidin-1-yl)ethyl)-2-(2,6-dioxopiperidin-3-yl)isoindoline-1,3-dione (6)

To a solution of tert-butyl (1-(2-(2-(2,6-dioxopiperidin-3-yl)-1,3-dioxoisoindolin-4-yl)ethyl)piperidin-4-yl)carbamate (100 mg, 206.38 μmol) in dioxane (2 mL) was added HCl/dioxane (4 M, 2 mL), the mixture was stirred at 25° C. for 1 h. LCMS showed a peak (79%) with desired mass. The reaction mixture was concentrated under reduced pressure to afford 4-(2-(4-aminopiperidin-1-yl)ethyl)-2-(2,6-dioxopiperidin-3-yl)isoindoline-1,3-dione (86 mg, crude, HCl) was obtained as a white solid. MS(M+H)⁺=385.0

### Step 6. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(2-(2,6-dioxopiperidin-3-yl)-1,3-dioxoisoindolin-4-yl)ethyl)piperidin-4-yl)-3-methoxybenzamide (Compound 13)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (85.05 mg, 190.07 μmol) in DMF (2 mL) were added HATU (108.41 mg, 285.11 μmol), DIPEA (73.70 mg, 570.22 μmol, 99.32 μL), the mixture was stirred at 25° C. for 0.5 h, 4-(2-(4-aminopiperidin-1-yl)ethyl)-2-(2,6-dioxopiperidin-3-yl)isoindoline-1,3-dione (80 mg, crude, HCl) was added, the mixture was stirred at 25° C. for 16 h. LCMS showed a peak (80%) with desired mass. The reaction mixture was diluted with H₂O (5 mL). The organic phase was separated, the aqueous phase was extracted with EtOAc (5 mL×2). The combined organic layers were washed with brine (5 mL×2), dried over Na₂SO₄, filtered, and the filtrate was concentrated under reduced pressure to give a residue. The residue was purified by prep-HPLC (column: Phenomenex Synergi C18 150*25 mm*10 μm; mobile phase: [water(FA)-ACN]; B %: 19%-55%, 12 min) followed by HPLC (column: Waters Xbridge 150*25 mm*5 μm; mobile phase: [water(NH₄HCO₃)-ACN]; B %: 40%-70%, 10 min) followed by prep-HPLC (column: Unisil 3-100 C18 Ultra 150*50 mm*3 μm; mobile phase: [water (FA)-ACN]; B %: 15%-35%, 10 min). The eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(2-(2,6-dioxopiperidin-3-yl)-1,3-dioxoisoindolin-4-yl)ethyl)piperidin-4-yl)-3-methoxybenzamide (64.1 mg, 77.19 μmol, 40.61% yield, 98% purity) as a white solid. MS(M+H)⁺=814.3

¹H NMR (400 MHz, DMSO-d₆) δ=11.13 (s, 1H), 8.35-8.23 (m, 2H), 8.11 (d, J=7.6 Hz, 1H), 7.96 (s, 1H), 7.81-7.72 (m, 3H), 7.53-7.44 (m, 2H), 5.18-5.10 (m, 1H), 4.81-4.70 (m, 1H), 4.04 (t, J=14.1 Hz, 2H), 3.93 (s, 3H), 3.84-3.71 (m, 1H), 3.32 (s, 3H), 3.25-3.21 (m, 2H), 3.00-2.94 (m, 2H), 2.91-2.83 (m, 1H), 2.64-2.54 (m, 4H), 2.13-2.02 (m, 3H), 1.99-1.89 (m, 2H), 1.82-1.75 (m, 2H), 1.74-1.66 (m, 2H), 1.65-1.52 (m, 6H).

### Example 14. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(2-(2,6-dioxopiperidin-3-yl)-1,3-dioxoisoindolin-4-yl)propyl)piperidin-4-yl)-3-methoxybenzamide (Compound 14)

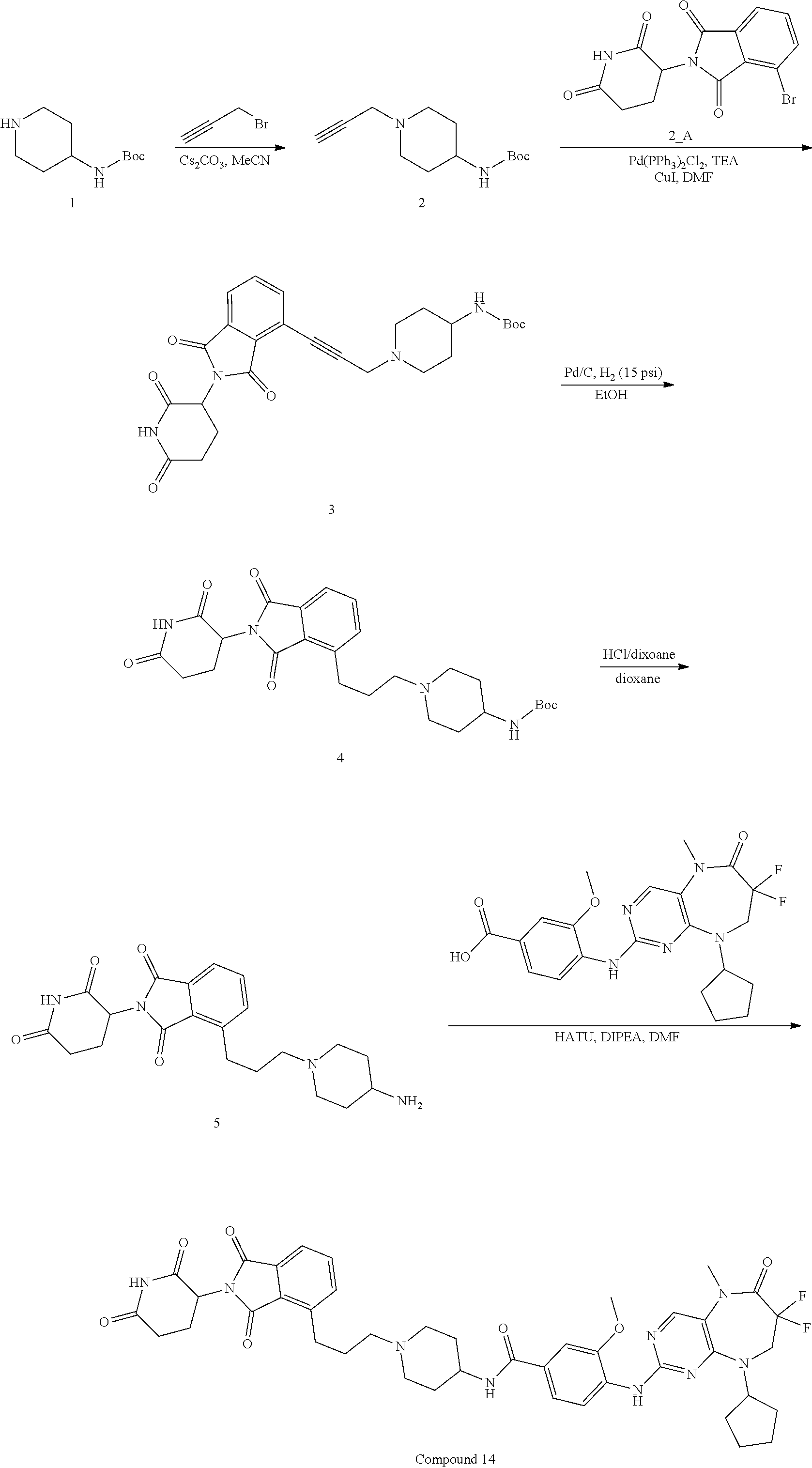

### Step 1. Synthesis of tert-butyl (1-(prop-2-yn-1-yl)piperidin-4-yl)carbamate (2)

To a solution of tert-butyl piperidin-4-ylcarbamate (2 g, 9.99 mmol) in ACN (20 mL) were added Cs₂CO₃ (6.51 g, 19.97 mmol) and 3-bromoprop-1-yne (1.48 g, 9.99 mmol, 1.08 mL, 80% purity), the mixture was stirred at 25° C. for 16 hr. TLC (100% EtOAc) showed a new spot by developing with KMnO₄. The mixture was diluted with water (10 mL), extracted with EtOAc (5 mL×3). The organic layer was dried over Na₂SO₄, filtered and concentrated in vacuum. The crude product was purified by flash silica gel chromatography (Biotage, 4 g SepaFlash® Silica Flash Column, Eluent of 4˜100% EtOAc/Petroleum ether gradient @20 mL/min) to afford tert-butyl (1-(prop-2-yn-1-yl)piperidin-4-yl)carbamate (1.4 g, 5.87 mmol, 58.82% yield) as a yellow powder. MS(M+H)⁺=239.3

### Step 2. Synthesis of tert-butyl (1-(3-(2-(2,6-dioxopiperidin-3-yl)-1,3-dioxoisoindolin-4-yl)prop-2-yn-1-yl)piperidin-4-yl)carbamate (3)

To a solution of 4-bromo-2-(2,6-dioxopiperidin-3-yl)isoindoline-1,3-dione (100 mg, 296.63 μmol) and tert-butyl (1-(prop-2-yn-1-yl)piperidin-4-yl)carbamate (84.83 mg, 355.95 μmol) in DMF (2 mL) were added Pd(PPh₃)₂Cl₂ (20.82 mg, 29.66 μmol), CuI (5.65 mg, 29.66 μmol) and TEA (300.15 mg, 2.97 mmol, 412.86 μL), the mixture was stirred at 80° C. for 16 hr. LCMS showed desired mass, the mixture was diluted with water (5 mL) and extracted with EtOAc (5 mL×3). The organic layer was dried over Na₂SO₄, filtered and concentrated under vacuum. The crude product was purified by flash silica gel chromatography (Biotage, 4 g SepaFlash® Silica Flash Column, Eluent of 4˜100% EtOAc/Petroleum ether gradient @35 mL/min) to afford tert-butyl (1-(3-(2-(2,6-dioxopiperidin-3-yl)-1,3-dioxoisoindolin-4-yl)prop-2-yn-1-yl)piperidin-4-yl)carbamate (130 mg, 252.36 μmol, 85.08% yield, 96% purity) as yellow oil. MS(M+H)⁺=495.0

### Step 3. Synthesis of tert-butyl (1-(3-(2-(2,6-dioxopiperidin-3-yl)-1,3-dioxoisoindolin-4-yl)propyl)piperidin-4-yl)carbamate (4)

A solution of tert-butyl (1-(3-(2-(2,6-dioxopiperidin-3-yl)-1,3-dioxoisoindolin-4-yl)prop-2-yn-1-yl)piperidin-4-yl)carbamate (130 mg, 262.87 μmol) in EtOH (2 mL) was added Pd/C (1.30 g, 10% purity) under N₂, then H₂ (529.90 g, 262.87 μmol) was bubbled into the mixture, the mixture was stirred under 15 psi of H₂ at 25° C. for 16 hr. LCMS showed main peak with desired mass, the mixture was filtered and concentrated under vacuum to afford tert-butyl (1-(3-(2-(2,6-dioxopiperidin-3-yl)-1,3-dioxoisoindolin-4-yl)propyl)piperidin-4-yl)carbamate (120 mg, crude) as yellow oil, which was used in the next step directly. MS(M+H)⁺=499.0

### Step 4. Synthesis of 4-(3-(4-aminopiperidin-1-yl)propyl)-2-(2,6-dioxopiperidin-3-yl)isoindoline-1,3-dione (5)

To a solution of tert-butyl (1-(3-(2-(2,6-dioxopiperidin-3-yl)-1,3-dioxoisoindolin-4-yl)propyl)piperidin-4-yl)carbamate (120 mg, 240.69 μmol) in dioxane (1 mL) was added HC/dioxane (4 M, 1 mL), the mixture was stirred at 25° C. for 1 hr. LCMS showed desired mass, the mixture was concentrated in vacuum to afford 4-(3-(4-aminopiperidin-1-yl)propyl)-2-(2,6-dioxopiperidin-3-yl)isoindoline-1,3-dione (90 mg, 225.87 μmol, 93.84% yield) as a yellow powder. MS(M+H)⁺=399.0

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(2-(2,6-dioxopiperidin-3-yl)-1,3-dioxoisoindolin-4-yl)propyl)piperidin-4-yl)-3-methoxybenzamide (Compound 14)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (90 mg, 201.15 μmol) in DMF (1 mL) were added HATU (114.72 mg, 301.72 μmol) and DIEA (77.99 mg, 603.44 μmol, 105.11 μL), after stirring at 25° C. for 0.5 h, then 4-(3-(4-aminopiperidin-1-yl)propyl)-2-(2,6-dioxopiperidin-3-yl)isoindoline-1,3-dione (87.48 mg, 201.15 μmol, HCl salt) was added and the resulting mixture was stirred at 25° C. for 16 h. LCMS showed desired mass. The mixture was diluted with water (3 mL) and extracted with EtOAc (5 mL×3). The organic layer was dried over Na₂SO₄, filtered and concentrated under vacuum, the crude product was purified by flash silica gel chromatography (Biotage, 4 g SepaFlash® Silica Flash Column, Eluent of 4˜10% MeOH/DCM@30 mL/min) followed by Prep-HPLC (column: Waters Xbridge 150*25 mm*5 μm; mobile phase: [water(NH₄HCO₃)-ACN]; B %: 45%-75%, 8 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(2-(2,6-dioxopiperidin-3-yl)-1,3-dioxoisoindolin-4-yl)propyl)piperidin-4-yl)-3-methoxybenzamide (7.8 mg, 9.20 μmol, 4.57% yield, 97.6% purity) as a white powder. MS(M+H)⁺=828.0

¹H NMR (400 MHz, DMSO-d₆) δ=11.13 (s, 1H), 8.24-8.30 (m, 2H), 8.11 (br d, J=7.58 Hz, 1H), 7.96 (s, 1H), 7.71-7.82 (m, 3H), 7.46-7.52 (m, 2H), 5.14 (dd, J=12.90, 5.44 Hz, 1H), 4.77 (t, J=8.07 Hz, 1H), 4.05 (t, J=14.12 Hz, 2H), 3.94 (s, 3H), 3.72-3.81 (m, 1H), 3.42 (s, 3H), 3.04-3.09 (m, 2H), 2.85-2.93 (m, 3H), 2.62-2.70 (m, 2H), 2.32-2.37 (m, 2H), 2.03-2.11 (m, 1H), 1.95 (t, J=10.58 Hz, 4H), 1.69-1.84 (m, 6H), 1.54-1.66 (m, 6H).

### Example 15. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-((3-((2-(2,6-dioxopiperidin-3-yl)-1,3-dioxoisoindolin-4-yl)oxy)propyl)(methyl)amino)cyclohexyl)-3-methoxybenzamide (trans) (Compound 15)

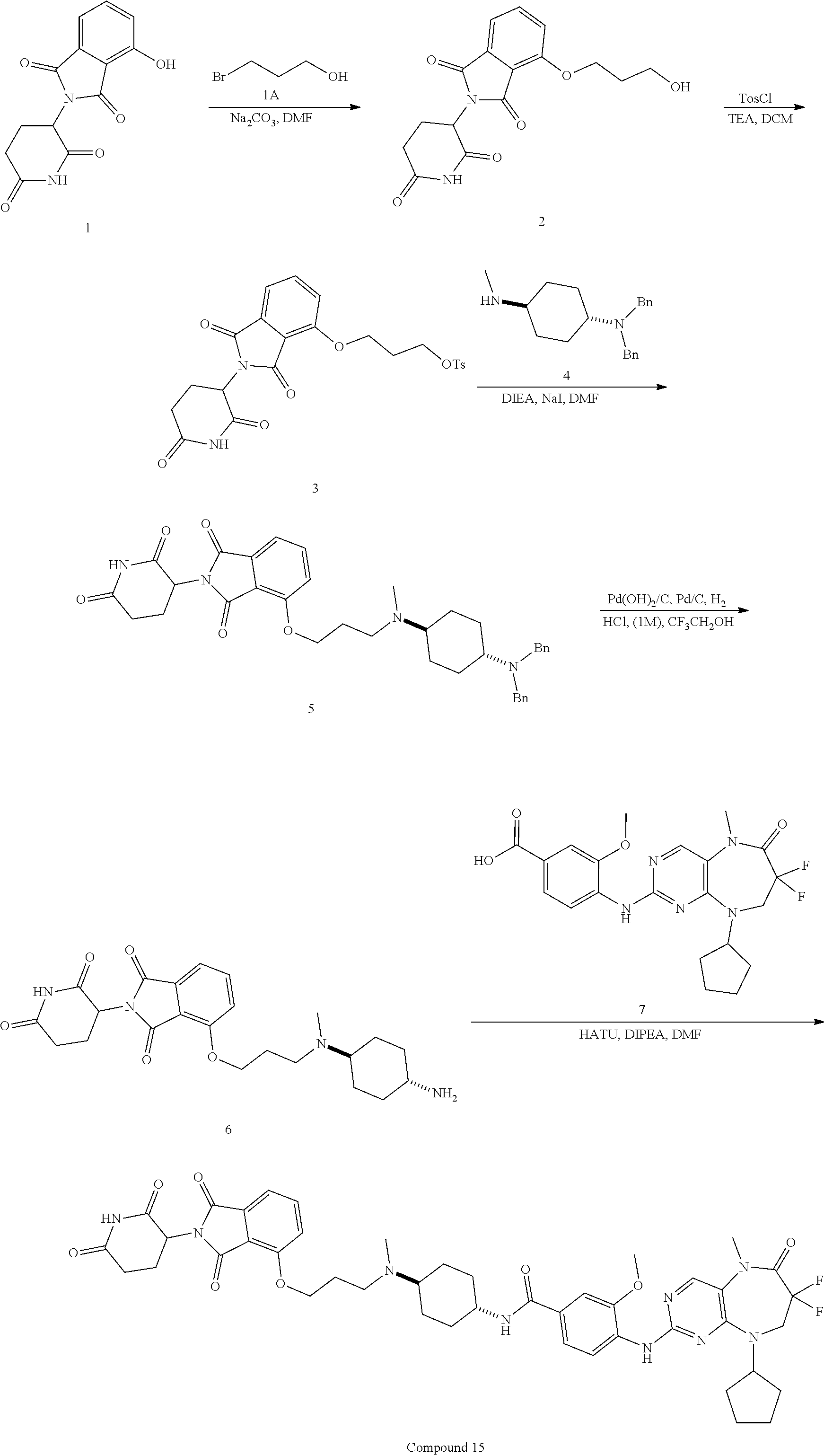

### Step 1. Synthesis of 2-(2,6-dioxopiperidin-3-yl)-4-(3-hydroxypropoxy)isoindoline-1,3-dione (2)

To a solution of 2-(2,6-dioxopiperidin-3-yl)-4-hydroxyisoindoline-1,3-dione (5 g, 18.23 mmol) and 3-bromopropan-1-ol (2.79 g, 20.06 mmol, 1.81 mL) in DMF (130 mL) was added Na₂CO₃ (9.66 g, 91.16 mmol), the mixture was stirred at 80° C. for 16 hours. LCMS showed desired mass. The reaction mixture was filtered and the filter cake was washed with EtOAc (60 mL). The filtrate was diluted with brine (600 mL) and extracted with EtOAc (400 mL×5). The combined organic layers were concentrated in vacuum to remove some solvent and the resulting mixture was washed with brine (300 mL×5), dried over Na₂SO₄, filtered and concentrated in vacuum. The residue was triturated with a mixture of MTBE (25 mL) and EtOAc (5 mL) for 10 minutes, the suspension was filtered and the filter cake was washed with MTBE (20 mL). The filter cake was collected and dried to afford 2-(2,6-dioxopiperidin-3-yl)-4-(3-hydroxypropoxy)isoindoline-1,3-dione (2.3 g, 6.92 mmol, 37.96% yield) as an off-white solid. MS(M+H)⁺=333.0

¹H NMR (400 MHz, DMSO-d₆) δ=11.10 (br s, 1H), 7.81 (t, J=7.9 Hz, 1H), 7.52 (d, J=8.5 Hz, 1H), 7.44 (d, J=7.3 Hz, 1H), 5.08 (dd, J=5.4, 12.8 Hz, 1H), 4.56 (t, J=5.1 Hz, 1H), 4.27 (t, J=6.2 Hz, 2H), 3.60 (q, J=5.8 Hz, 2H), 2.95-2.81 (m, 1H), 2.63-2.52 (m, 2H), 2.09-1.99 (m, 1H), 1.95-1.85 (m, 2H).

### Step 2. Synthesis of 3-((2-(2,6-dioxopiperidin-3-yl)-1,3-dioxoisoindolin-4-yl)oxy)propyl 4-methylbenzenesulfonate (3)

To a solution of 2-(2,6-dioxopiperidin-3-yl)-4-(3-hydroxypropoxy)isoindoline-1,3-dione (1 g, 3.01 mmol) in DCM (20 mL) were added TEA (1.22 g, 12.04 mmol, 1.68 mL) and TosCl (1.15 g, 6.02 mmol), the mixture was stirred at 25° C. for 16 hours. LCMS s showed 17% of the starting material remained and 65% of desired mass. The resulting mixture was concentrated in vacuum. The residue was purified by flash silica gel chromatography (20 g SepaFlash® Silica Flash Column, Eluent of from 25˜100% EtOAc/Petroleum ether to 10-17% Methanol/EtOAc gradient @100 mL/min). The eluent was concentrated in vacuum to afford the crude product. The crude product was purified by prep-HPLC (column: Phenomenex luna C18 150*40 mm*15 μm; mobile phase: [water(FA)-ACN]; B %: 32%-62%, 10 min) and the eluent was freeze-dried to afford 3-((2-(2,6-dioxopiperidin-3-yl)-1,3-dioxoisoindolin-4-yl)oxy)propyl 4-methylbenzenesulfonate (490 mg, 1.01 mmol, 33.47% yield) as a white solid. MS(M+H)⁺=487.0.

¹H NMR (400 MHz, DMSO-d₆) δ=11.13 (s, 1H), 7.77 (dd, J=7.5, 8.3 Hz, 1H), 7.68 (d, J=8.3 Hz, 2H), 7.45 (d, J=7.3 Hz, 1H), 7.34 (d, J=8.6 Hz, 1H), 7.26 (d, J=8.1 Hz, 2H), 5.10 (dd, J=5.5, 12.7 Hz, 1H), 4.28 (t, J=5.8 Hz, 2H), 4.07 (t, J=5.6 Hz, 2H), 2.96-2.82 (m, 1H), 2.65-2.53 (m, 2H), 2.20 (s, 3H), 2.12-1.95 (m, 3H).

### Step 3. Synthesis of 4-(3-(((1r,4r)-4-(dibenzylamino)cyclohexyl)(methyl)amino)propoxy)-2-(2,6-dioxopiperidin-3-yl)isoindoline-1,3-dione (trans) (5)

To a solution of 3-((2-(2,6-dioxopiperidin-3-yl)-1,3-dioxoisoindolin-4-yl)oxy)propyl 4-methylbenzenesulfonate (245 mg, 503.60 μmol) and (1r,4r)-N1,N1-dibenzyl-N4-methylcyclohexane-1,4-diamine (trans) (310.68 mg, 1.01 mmol) in DMF (6 mL) were added DIPEA (260.34 mg, 2.01 mmol, 350.87 μL) and NaI (7.55 mg, 50.36 μmol), the mixture was stirred at 60° C. for 16 hours. LCMS showed the starting material was consumed completely and the desired mass. The resulting mixture was diluted with H₂O (60 mL) and extracted with EtOAc (40 mL×3). The combined organic layers were washed with brine (20 mL×5), dried over Na₂SO₄, filtered and concentrated in vacuum. The residue was purified by pre-TLC (SiO₂, DCM:Methanol=5:1) to afford 4-(3-(((1r,4r)-4-(dibenzylamino)cyclohexyl)(methyl)amino)propoxy)-2-(2,6-dioxopiperidin-3-yl)isoindoline-1,3-dione (trans) (195 mg, 313.13 μmol, 62.18% yield) as a brown solid. MS(M+H)⁺=623.2

### Step 4. Synthesis of 4-(3-(((1r,4r)-4-aminocyclohexyl)(methyl)amino)propoxy)-2-(2,6-dioxopiperidin-3-yl)isoindoline-1,3-dione (trans) (6)

To a solution of 4-(3-(((1r,4r)-4-(dibenzylamino)cyclohexyl)(methyl)amino)propoxy)-2-(2,6-dioxopiperidin-3-yl)isoindoline-1,3-dione (trans) (185.00 mg, 297.07 μmol) in CF₃CH₂OH (30 mL) were added Pd(OH)₂/C (100 mg, 10% purity), HCl (1 M in H₂O, 297.07 μL) and Pd/C (100 mg, 10% purity) under N₂ atmosphere, the mixture was degassed and purged with H₂ for 3 times and the suspension was stirred at 50° C. under H₂ (15 psi) atmosphere for 16 hours. LCMS showed the starting material was consumed completely and a main peak with desired mass. The reaction mixture was filtered and the filter cake was washed with CF₃CH₂OH (50 mL), the filtrate was concentrated in vacuum. The residue was purified by prep-TLC (SiO₂, DCM:Methanol=5:1) to afford 4-(3-(((1r,4r)-4-aminocyclohexyl)(methyl) amino)propoxy)-2-(2,6-dioxopiperidin-3-yl)isoindoline-1,3-dione (trans) (180 mg, HCl salt) as a brown solid. MS(M+H)⁺=443.1

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-((3-((2-(2,6-dioxopiperidin-3-yl)-1,3-dioxoisoindolin-4-yl)oxy)propyl)(methyl)amino)cyclohexyl)-3-methoxybenzamide (trans) (Compound 15)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (105 mg, 234.67 μmol) in DMF (3 mL) were added HATU (116.00 mg, 305.07 μmol) and DIPEA (151.65 mg, 1.17 mmol, 204.38 μL), the mixture was stirred at 25° C. for 15 minutes. To the mixture was added 4-(3-(((1r,4r)-4-aminocyclohexyl)(methyl)amino)propoxy)-2-(2,6-dioxopiperidin-3-yl) isoindoline-1,3-dione (179.84 mg, 375.47 μmol, HCl salt) and the resulting mixture was stirred at 25° C. for 1 hour. LCMS showed the starting material was consumed completely and the desired mass. To the mixture was added CH₃COOH to adjust pH<7. The resulting mixture was purified by prep-HPLC (column: Phenomenex C18 75*30 mm*3 μm; mobile phase: [water(FA)-ACN]; B %: 18%-48%, 7 min) followed by prep-HPLC (column: Waters Xbridge 150*25 mm*5 μm; mobile phase: [water(NH₄HCO₃)-ACN]; B %: 48%-78%, 8 min) and prep-HPLC (column: Phenomenex C18 75*30 mm*3 μm; mobile phase: [water(FA)-ACN]; B %: 18%-48%, 7 min), the eluent was freeze-dried to afford 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-((3-((2-(2,6-dioxopiperidin-3-yl)-1,3-dioxoisoindolin-4-yl)oxy)propyl)(methyl)amino)cyclohexyl)-3-methoxybenzamide (7.4 mg, 8.32 μmol, 3.54% yield, 98% purity) as a white solid. MS (M+H)⁺=872.3

¹H NMR (400 MHz, DMSO-d₆) δ=11.09 (s, 1H), 8.33-8.24 (m, 2H), 8.06 (br d, J=7.9 Hz, 1H), 7.95 (s, 1H), 7.87-7.76 (m, 1H), 7.61-7.38 (m, 4H), 5.10 (dd, J=5.4, 12.8 Hz, 1H), 4.87-4.66 (m, 1H), 4.25 (br t, J=5.9 Hz, 2H), 4.04 (br t, J=14.1 Hz, 2H), 3.93 (s, 3H), 3.71-3.60 (m, 1H), 3.32 (s, 3H), 2.95-2.85 (m, 1H), 2.65-2.58 (m, 4H), 2.45-2.35 (m, 1H), 2.23 (s, 3H), 2.07-2.00 (m, 1H), 1.95-1.87 (m, 2H), 1.91-1.81 (m, 4H), 1.75-1.64 (m, 4H), 1.65-1.51 (m, 4H), 1.48-1.26 (m, 4H).

### Example 16. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1s,4s)-4-((3-((2-(2,6-dioxopiperidin-3-yl)-1,3-dioxoisoindolin-4-yl)amino)propyl)(methyl)amino)cyclohexyl)-3-methoxybenzamide (Compound 16)

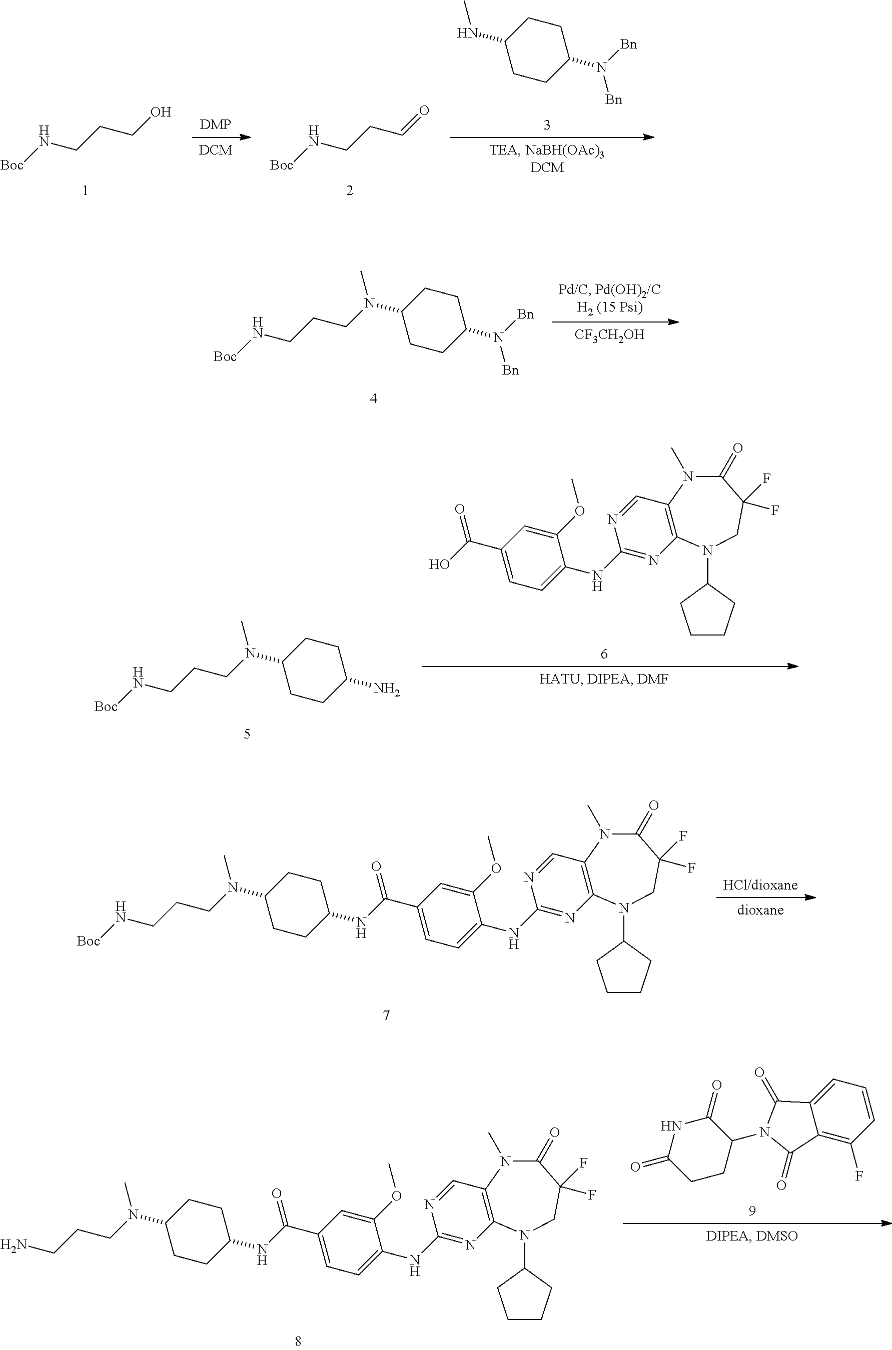

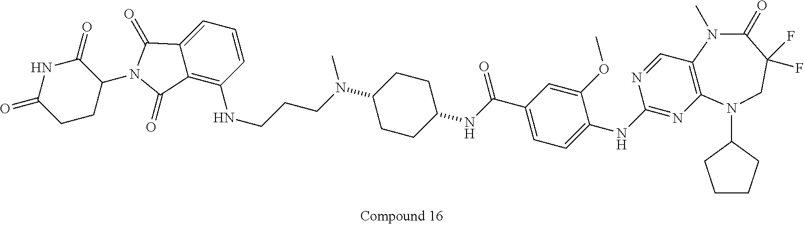 [SMILES: COc1cc(C(=O)N[C@H]2CC[C@@H](N(C)CCCNc3cccc4c3C(=O)N(C3CCC(=O)NC3=O)C4=O)CC2)ccc1Nc1ncc2c(n1)N(C1CCCC1)CC(F)(F)C(=O)N2C]

The compound 16 was synthesized by the method described in the scheme similar to the method described in Example 15.

MS(M+H)⁺=871.1, ¹H NMR (400 MHz, DMSO-d₆) δ=11.07 (s, 1H), 8.31-8.22 (m, 2H), 8.02-7.90 (m, 2H), 7.63-7.54 (m, 1H), 7.52-7.45 (m, 2H), 7.11 (d, J=8.6 Hz, 1H), 7.02 (d, J=7.1 Hz, 1H), 6.78 (t, J=5.8 Hz, 1H), 5.09-4.99 (m, 1H), 4.83-4.70 (m, 1H), 4.10-3.98 (m, 2H), 3.98-3.88 (m, 4H), 3.35-3.31 (m, 5H), 2.93-2.80 (m, 1H), 2.61-2.53 (m, 4H), 2.37-2.30 (m, 1H), 2.22 (s, 3H), 2.04-1.90 (m, 3H), 1.87-1.67 (m, 8H), 1.65-1.43 (m, 8H).

### Example 17. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(20-((2-(2,6-dioxopiperidin-3-yl)-1,3-dioxoisoindolin-4-yl)amino)-3,6,9,12,15,18-hexaoxaicosyl)piperidin-4-yl)-2-fluoro-5-methoxybenzamide (Compound 17)

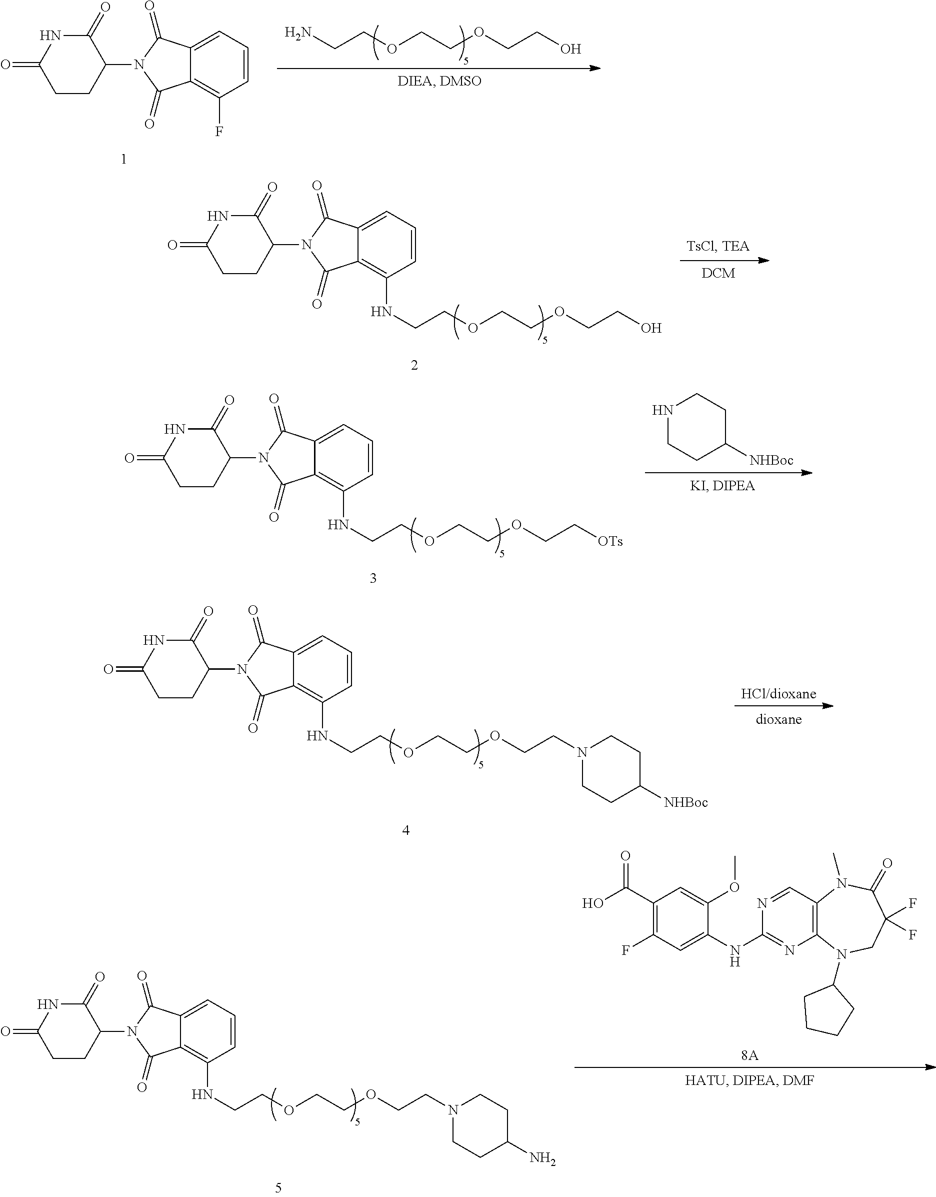

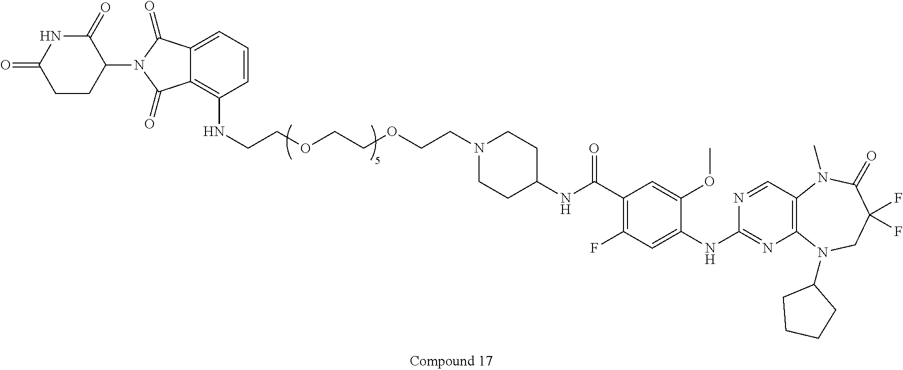

The compound 17 was synthesized by the method described in the scheme similar to the method described in Example 1.

MS(M+H)⁺=1111.9, ¹H NMR (400 MHz, DMSO-d₆) δ 11.09 (s, 1H), 8.30 (s, 1H), 8.24 (d, J=13.3 Hz, 1H), 8.03 (s, 1H), 7.91-7.83 (m, 1H), 7.62-7.52 (m, 1H), 7.19 (d, J=6.7 Hz, 1H), 7.14 (d, J=8.5 Hz, 1H), 7.03 (d, J=7.0 Hz, 1H), 6.60 (t, J=5.8 Hz, 1H), 5.11-4.99 (m, 1H), 4.87-4.76 (m, 1H), 4.13-3.99 (m, 2H), 3.91 (s, 3H), 3.78-3.68 (m, 1H), 3.64-3.59 (m, 2H), 3.57-3.44 (m, 24H), 3.33 (s, 3H), 2.94-2.80 (m, 3H), 2.62-2.53 (m, 2H), 2.48-2.44 (m, 2H), 2.10-1.90 (m, 5H), 1.81-1.68 (m, 4H), 1.67-1.48 (m, 6H).

### Example 18. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(2-(2-((2-(2,6-dioxopiperidin-3-yl)-1-oxoisoindolin-4-yl)amino)ethoxy)ethoxy)ethyl)piperidin-4-yl)-3-methoxybenzamide (Compound 18)

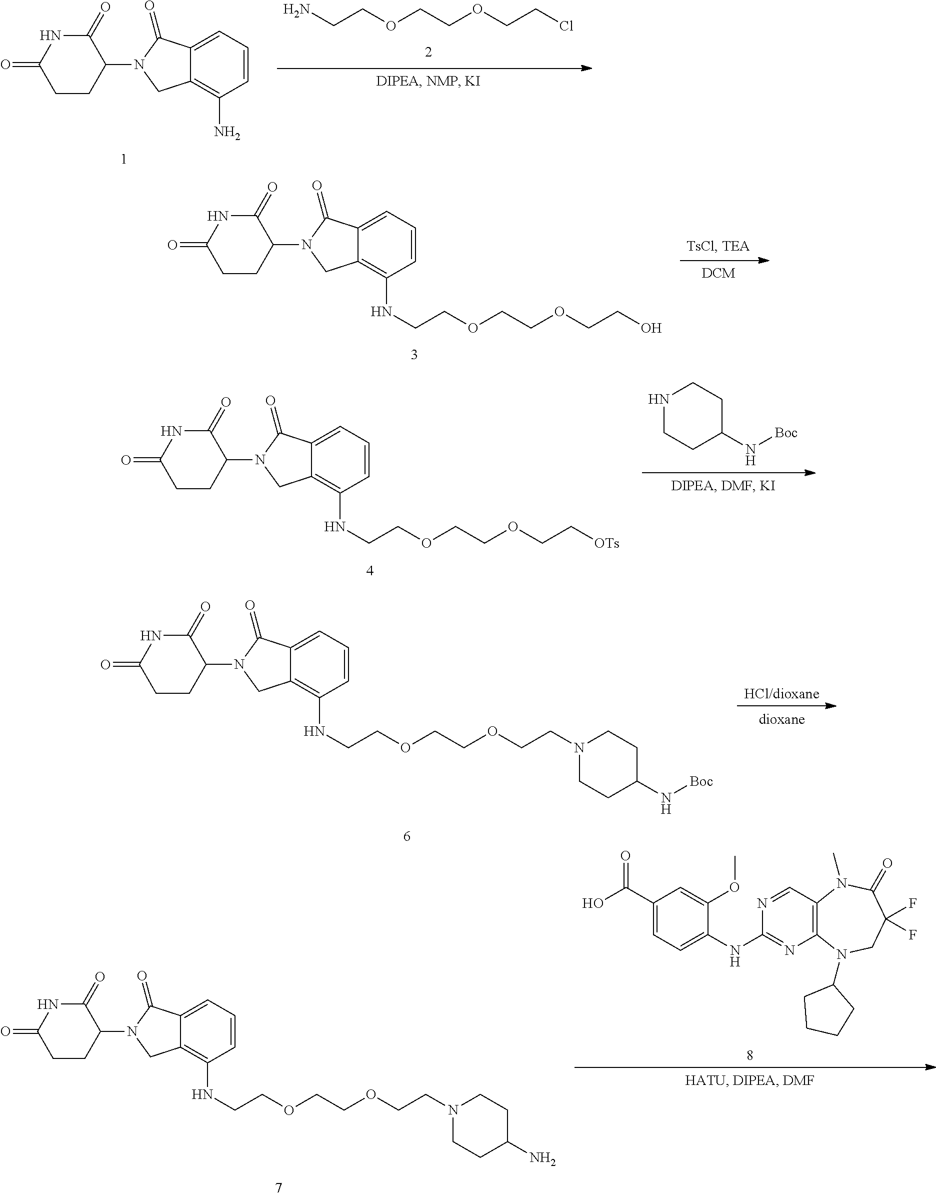

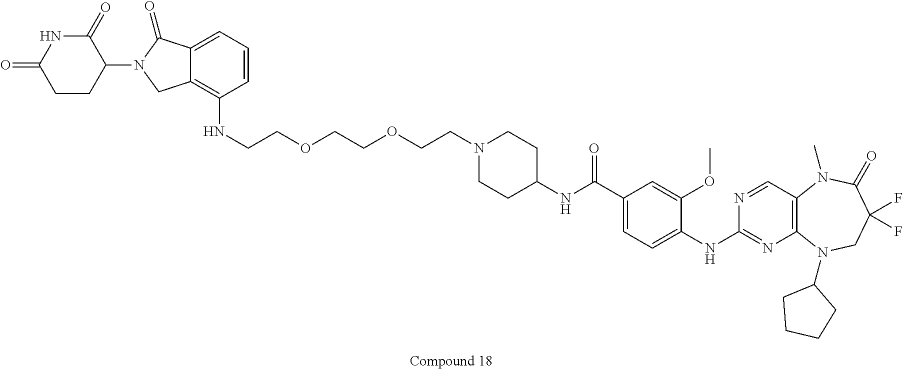 [SMILES: COc1cc(C(=O)NC2CCN(CCOCCOCCNc3cccc4c3CN(C3CCC(=O)NC3=O)C4=O)CC2)ccc1Nc1ncc2c(n1)N(C1CCCC1)CC(F)(F)C(=O)N2C]

The compound 18 was synthesized by the method described in the scheme similar to the method described in Example 1.

MS(M+H)⁺=903.7, ¹H NMR (400 MHz, DMSO-d₆) δ 11.01 (s, 1H), 8.33-8.26 (m, 2H), 8.15-8.05 (m, 1H), 7.97 (s, 1H), 7.52-7.47 (m, 2H), 7.30 (t, J=7.7 Hz, 1H), 6.95 (d, J=7.4 Hz, 1H), 6.82 (d, J=7.9 Hz, 1H), 5.60 (t, J=5.8 Hz, 1H), 5.15-5.05 (m, 1H), 4.81-4.72 (m, 1H), 4.27-4.01 (m, 4H), 3.94 (s, 3H), 3.78-3.71 (m, 1H), 3.61 (t, J=5.9 Hz, 2H), 3.58-3.49 (m, 7H), 3.31 (s, 3H), 2.93-2.86 (m, 3H), 2.65-2.58 (m, 2H), 2.46-2.42 (m, 2H), 2.34-2.29 (m, 1H), 2.07-1.94 (m, 5H), 1.83-1.65 (m, 4H), 1.64-1.50 (m, 6H).

### Example 19. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(2-(2-((2-(2,6-dioxopiperidin-3-yl)-1-oxoisoindolin-4-yl)oxy)ethoxy)ethoxy)ethyl)piperidin-4-yl)-3-methoxybenzamide (Compound 19)

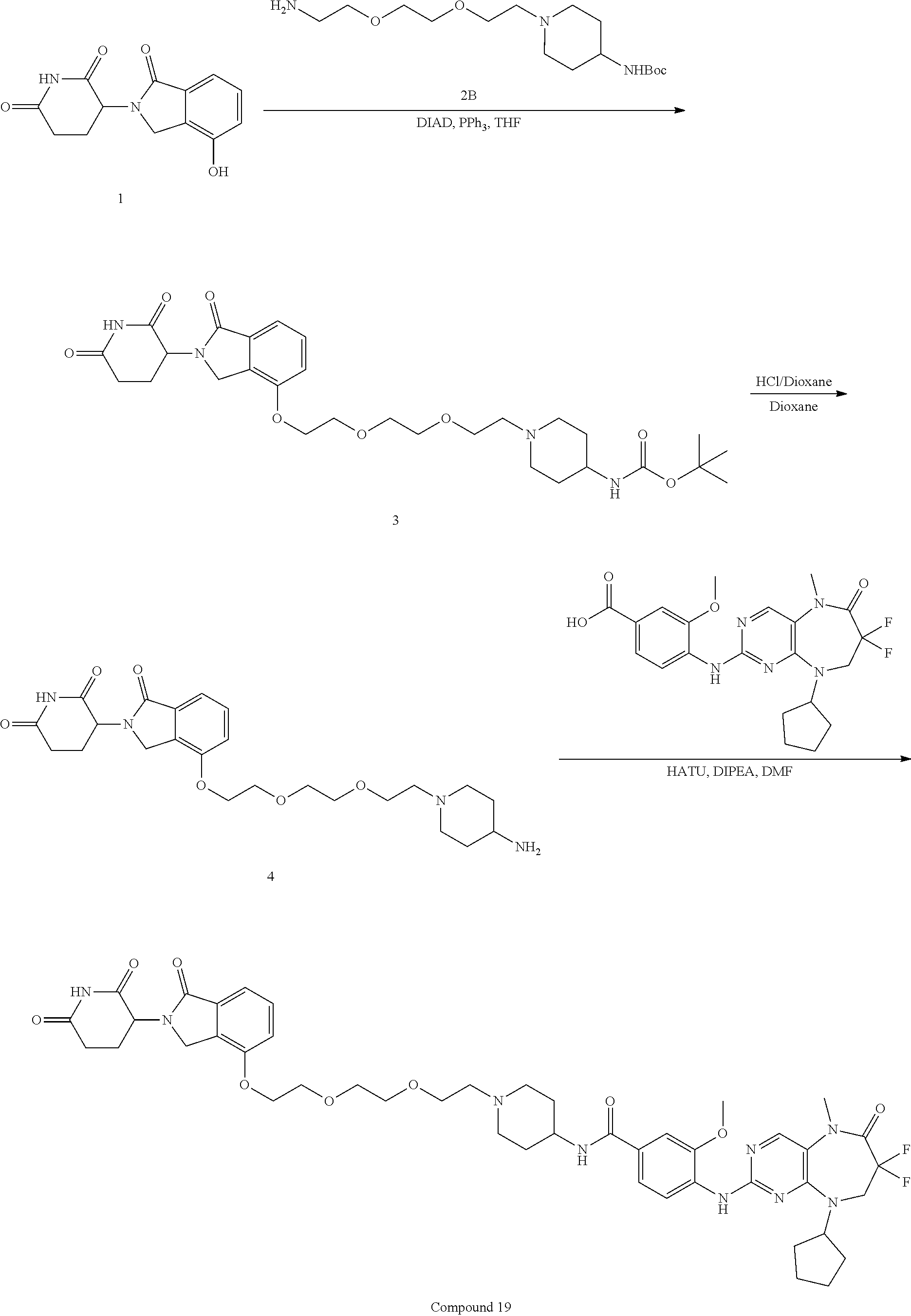

The compound 19 was synthesized by the method described in the scheme similar to the method described in Example 1.

MS (M+H)⁺=904.7, ¹H NMR (400 MHz, DMSO-d₆) δ 11.05-10.91 (m, 1H), 10.97 (s, 1H), 8.33-8.23 (m, 2H), 8.15-8.07 (m, 1H), 7.97 (s, 1H), 7.55-7.44 (m, 3H), 7.39-7.25 (m, 2H), 5.15-5.01 (m, 1H), 4.86-4.69 (m, 1H), 4.41-4.35 (m, 1H), 4.29-4.20 (m, 3H), 4.09-3.99 (m, 2H), 3.94 (s, 3H), 3.82-3.70 (m, 3H), 3.65-3.59 (m, 2H), 3.57-3.49 (m, 4H), 3.32-3.29 (m, 3H), 2.96-2.83 (m, 3H), 2.60-2.56 (m, 1H), 2.48-2.43 (m, 3H), 2.07-1.91 (m, 5H), 1.79-1.67 (m, 4H), 1.67-1.52 (m, 6H).

### Example 20. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(3-(3-(2-((2-(2,6-dioxopiperidin-3-yl)-1-oxoisoindolin-4-yl)oxy)ethoxy)propoxy)propyl)-3-methoxybenzamide (Compound 20)

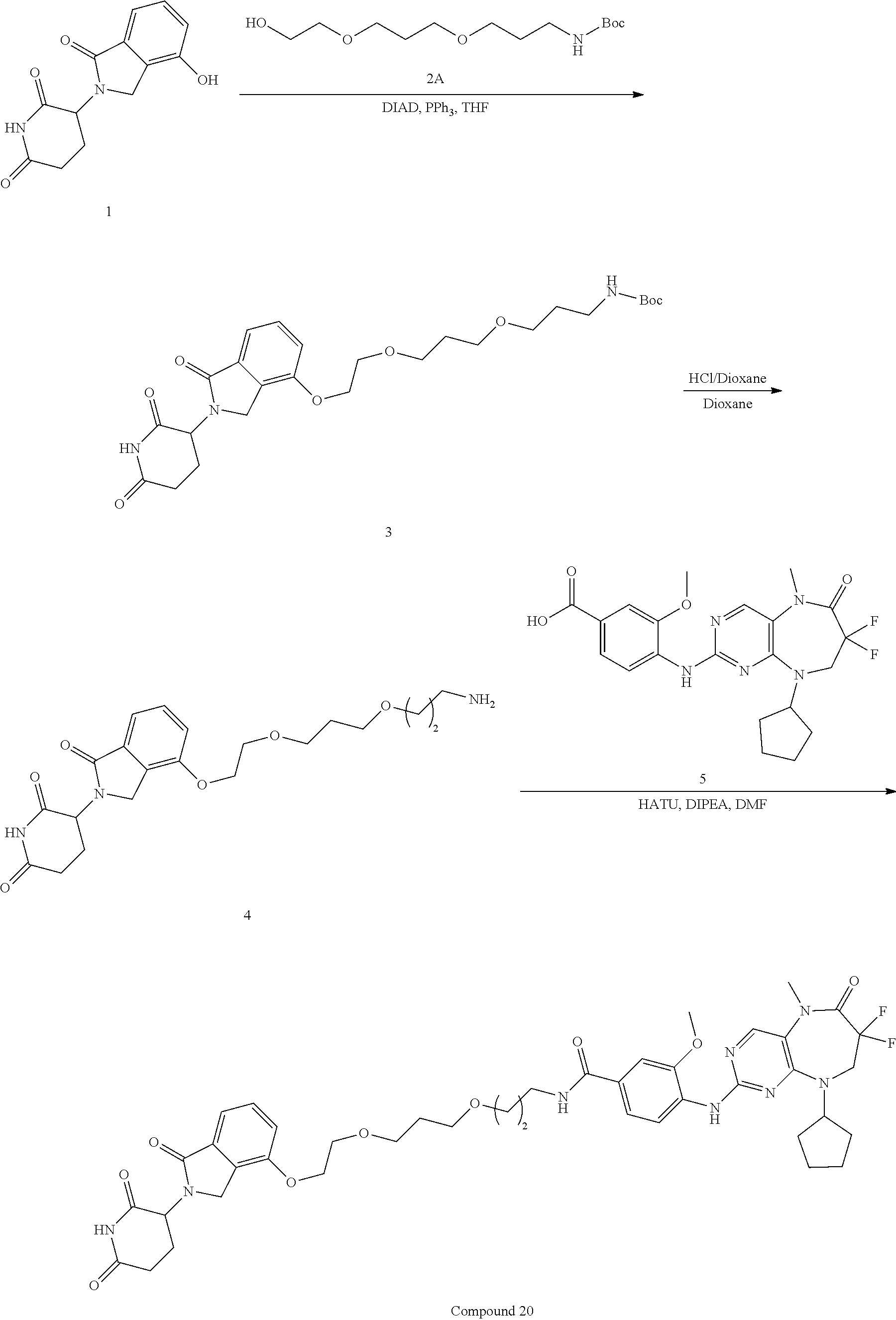

The compound 20 was synthesized by the method described in the scheme similar to the method described in Example 1.

MS (M+H)⁺=849.6, ¹H NMR (400 MHz, DMSO-d₆) δ=10.94 (s, 1H), 8.39-8.31 (m, 1H), 8.30-8.20 (m, 2H), 7.97 (s, 1H), 7.55-7.45 (m, 3H), 7.36-7.21 (m, 2H), 5.16-5.07 (m, 1H), 4.81-4.71 (m, 1H), 4.41-4.32 (m, 1H), 4.28-4.20 (m, 3H), 4.12-3.99 (m, 2H), 3.93 (s, 3H), 3.76-3.69 (m, 2H), 3.52 (t, J=6.5 Hz, 2H), 3.45-3.37 (m, 6H), 3.32-3.31 (m, 3H), 2.96-2.84 (m, 1H), 2.61-2.56 (m, 1H), 2.48-2.41 (m, 1H), 2.02-1.88 (m, 3H), 1.80-1.68 (m, 6H), 1.65-1.55 (m, 4H).

### Example 21. Synthesis of (S)-4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-(((2-(2,6-dioxopiperidin-3-yl)-1-oxoisoindolin-4-yl)oxy)methyl)benzyl)piperidin-4-yl)-3-methoxybenzamide (Compound 21)

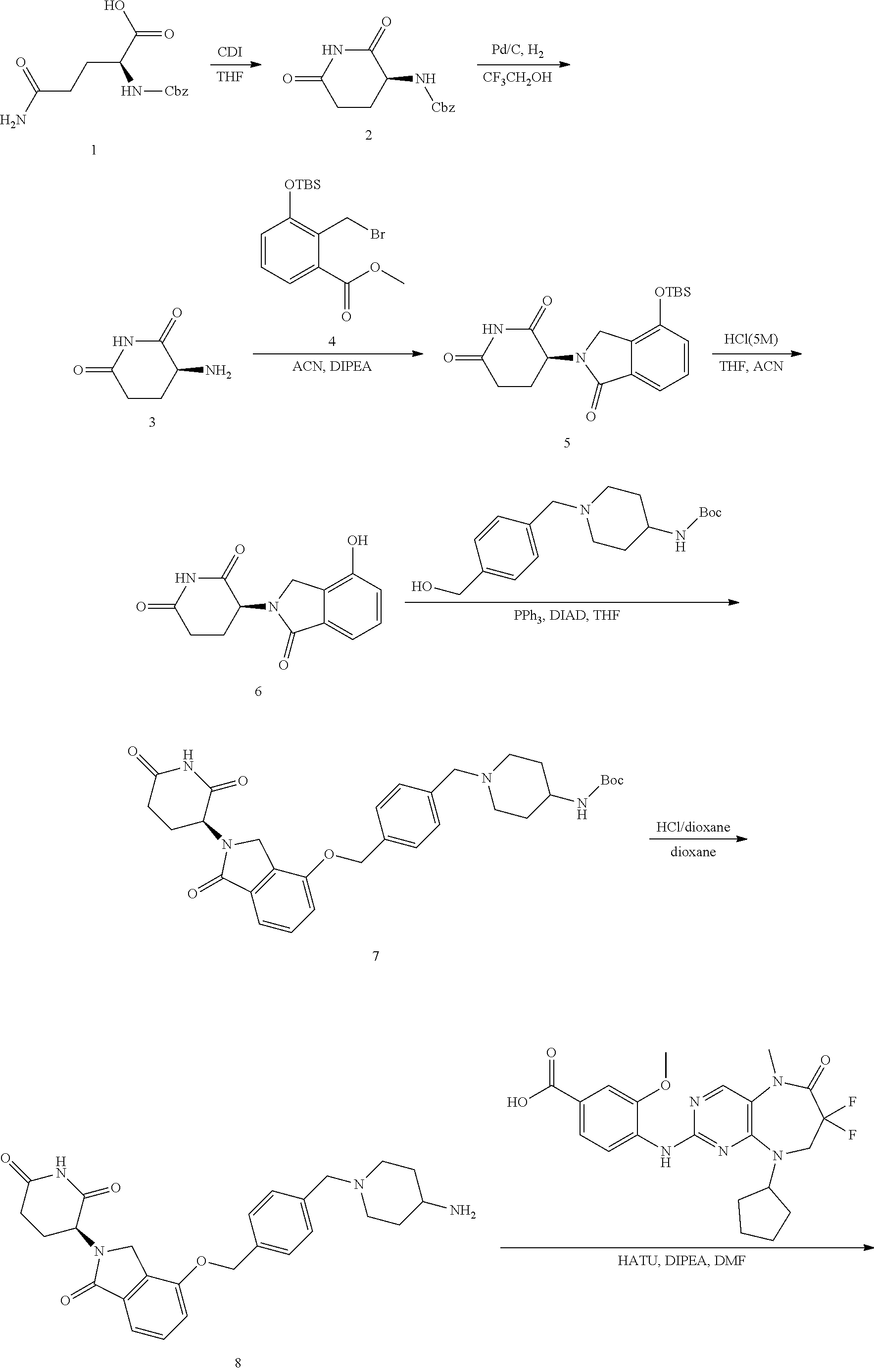

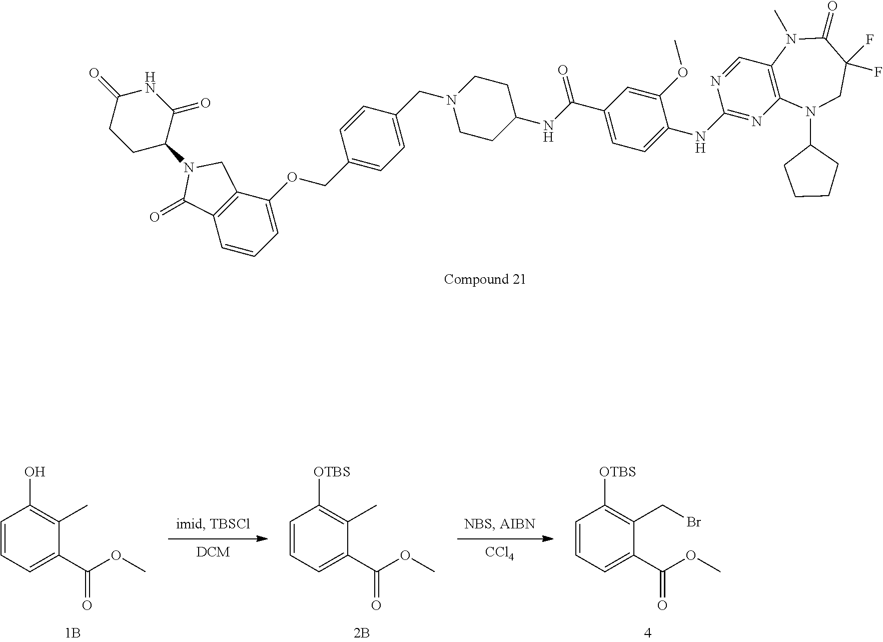

### Step 1. Synthesis of (S)-benzyl (2,6-dioxopiperidin-3-yl)carbamate (2)

To a solution of (S)-5-amino-2-(((benzyloxy)carbonyl)amino)-5-oxopentanoic acid (24 g, 85.63 mmol) in THF (400 mL) was added CDI (20 g, 123.34 mmol) at 25° C. The mixture was stirred at 70° C. for 16 hr under N₂ atmosphere. LCMS showed main peak with the desired mass. The reaction mixture was concentrated, then it was diluted with CHCl₃ (500 mL). The mixture was washed with brine 900 mL (300 mL×3), dried over Na₂SO₄, filtered and the filtrate was concentrated under reduced pressure. The crude product was triturated with MTBE (300 mL) at 25° C. for 30 min, filtered and the cake was dried under reduced pressure to afford (S)-benzyl (2,6-dioxopiperidin-3-yl)carbamate (16.5 g, 62.91 mmol, 73.47% yield) as a white solid. MS(M+H)⁺=263.2

### Step 2. Synthesis of (S)-3-aminopiperidine-2,6-dione (3)

To a solution of (S)-benzyl (2,6-dioxopiperidin-3-yl)carbamate (5 g, 19.06 mmol) in CF₃CH₂OH (200 mL) was added Pd/C (1 g, 10% purity) under N₂ atmosphere, then the mixture was stirred at 20° C. for 12 hr under H₂ atmosphere (15 Psi). LCMS showed main peak with the desired mass was detected and no peak with the starting material. The reaction mixture was filtered and the filtrate was concentrated under reduced pressure to afford (S)-3-aminopiperidine-2,6-dione (2.4 g, 18.73 mmol, 98.25% yield) as a gray solid. MS (M+H)⁺=129.2

### Step 3. Synthesis of methyl 3-((tert-butyldimethylsilyl)oxy)-2-methylbenzoate (2B)

To a solution of methyl 3-hydroxy-2-methylbenzoate (6 g, 36.11 mmol, 1 eq) in DCM (120 mL) were added imidazole (2.46 g, 36.11 mmol) and TBSCl (9 g, 59.71 mmol, 7.32 mL) at 25° C. The mixture was stirred at 25° C. for 16 hr under N₂ atmosphere. LCMS showed main peak with the desired mass was detected and no peak with the starting material. The reaction mixture was concentrated to give a residue and then H₂O (80 mL) was added, the mixture was extracted with EtOAc 300 mL (100 mL×3), then the combined organic layers were washed with brine (100 mL×2), dried over Na₂SO₄, filtered and the filtrate was concentrated under reduced pressure. The residue was purified by flash silica gel chromatography (Biotage; 40 g SepaFlash® Silica Flash Column, Eluent of 0˜20% EtOAc:Petroleum ether gradient, 60 mL/min) to afford methyl 3-((tert-butyldimethylsilyl)oxy)-2-methylbenzoate (10 g, 35.66 mmol, 98.76% yield) as a light yellow oil. MS(M+H)⁺=281.2

### Step 4. Synthesis of methyl 2-(bromomethyl)-3-((tert-butyldimethylsilyl)oxy)benzoate (4)

To a solution of methyl 3-((tert-butyldimethylsilyl)oxy)-2-methylbenzoate (4 g, 14.26 mmol) in CCl₄ (15 mL) were added NBS (2.80 g, 15.73 mmol) and AIBN (240.00 mg, 1.46 mmol) at 25° C. The mixture was stirred at 80° C. for 16 hr under N₂ atmosphere. LCMS showed main peak with the desired mass was detected and no peak with the starting material. To the reaction mixture was added Na₂SO₃ (sat. aq, 40 mL) and extracted with DCM (40 mL×3), the combined organic layers were dried over Na₂SO₄, filtered and the filtrate was concentrated under reduced pressure. The residue was purified by flash silica gel chromatography (Biotage; 40 g SepaFlash® Silica Flash Column, Eluent of 0˜20% EtOAc:Petroleum ether gradient, 60 mL/min) to afford methyl 2-(bromomethyl)-3-((tert-butyldimethylsilyl)oxy)benzoate (5 g, 13.91 mmol, 97.55% yield) as a light yellow oil. MS(M+H)⁺=359.1

### Step 5. Synthesis of (S)-3-(4-((tert-butyldimethylsilyl)oxy)-1-oxoisoindolin-2-yl)piperidine-2,6-dione (5)

To a solution of (S)-3-aminopiperidine-2,6-dione (600 mg, 4.68 mmol) in ACN (20 mL) were added DIPEA (2.23 g, 17.22 mmol, 3.0 mL) and methyl 2-(bromomethyl)-3-((tert-butyldimethylsilyl)oxy)benzoate (1.86 g, 5.18 mmol) at 25° C. The mixture was stirred at 90° C. for 16 hr under N₂ atmosphere. LCMS showed 75% peak with the desired mass was detected and no peak with the starting material. The reaction mixture was concentrated under reduced pressure. H₂O (10 mL) and MTBE (15 mL) were added and the mixture was stirred at 25° C. for 1 hr, Then it was filtrated and the filter cake was washed with MTBE (10 mL×2) and dried under reduced pressure to afford (S)-3-(4-((tert-butyldimethylsilyl)oxy)-1-oxoisoindolin-2-yl)piperidine-2,6-dione (1.1 g, crude) as a gray solid. MS(M+H)⁺=375.2

SFC indicated two peak (49.6%: 50.3%) and the product maybe was a racemization.

### Step 6. Synthesis of (S)-3-(4-hydroxy-1-oxoisoindolin-2-yl)piperidine-2,6-dione (6)

To a solution of (S)-3-(4-((tert-butyldimethylsilyl)oxy)-1-oxoisoindolin-2-yl)piperidine-2,6-dione (500 mg, 1.34 mmol) in THF (5 mL) and CH₃CN (5 mL) were added HCl (5 M, 10.00 mL) at 0° C. The mixture was stirred at 25° C. for 16 hr under N₂ atmosphere. LCMS showed main peak with the desired mass. The reaction mixture was concentrated under reduced pressure and followed by lyophilization to afford (S)-3-(4-hydroxy-1-oxoisoindolin-2-yl)piperidine-2,6-dione (340 mg, crude) as a light yellow solid. MS(M+H)⁺=261.2

### Step 7. Synthesis of (S)-tert-butyl (1-(4-(((2-(2,6-dioxopiperidin-3-yl)-1-oxoisoindolin-4-yl)oxy)methyl)benzyl)piperidin-4-yl)carbamate (7)

To a solution of (S)-3-(4-hydroxy-1-oxoisoindolin-2-yl)piperidine-2,6-dione (300 mg, 1.15 mmol) in THF (50 mL) were added PPh₃ (907.06 mg, 3.46 mmol), tert-butyl (1-(4-(hydroxymethyl)benzyl)piperidin-4-yl)carbamate (420 mg, 1.31 mmol) and DIAD (1.23 g, 6.09 mmol, 1.18 mL) at 0° C. The mixture was stirred at 25° C. for 16 hr under N₂ atmosphere. LCMS showed 8% peak with the desired mass. The reaction mixture was concentrated under reduced pressure to afford (S)-tert-butyl (1-(4-(((2-(2,6-dioxopiperidin-3-yl)-1-oxoisoindolin-4-yl)oxy)methyl)benzyl)piperidin-4-yl)carbamate (600 mg, crude) as a light yellow oil. MS(M+H)⁺=563.4

### Step 8. Synthesis of (S)-3-(4-((4-((4-aminopiperidin-1-yl)methyl)benzyl)oxy)-1-oxoisoindolin-2-yl)piperidine-2,6-dione (8)

To a solution of (S)-tert-butyl (1-(4-(((2-(2,6-dioxopiperidin-3-yl)-1-oxoisoindolin-4-yl)oxy)methyl)benzyl)piperidin-4-yl)carbamate (600 mg, 1.07 mmol) in dioxane (10 mL) were added HCl/dioxane (4 M, 12.00 mL) at 25° C. The mixture was stirred at 25° C. for 1 hr under N₂ atmosphere. LCMS showed 13% peak with the desired mass was detected and no peak with the starting material. The reaction mixture was concentrated under reduced pressure. To the residue was added H₂O (30 mL), the mixture was washed with EtOAc (30 mL×3), the aqueous phase was freeze dried and purified by prep-HPLC (column: Phenomenex Synergi Polar-RP 100*25 mm*4 μm; mobile phase: [water(TFA)-ACN]; B %: 13%-33%, 7 min; Column Temp: 30° C.) followed by lyophilization to afford (S)-3-(4-((4-((4-aminopiperidin-1-yl)methyl)benzyl)oxy)-1-oxoisoindolin-2-yl)piperidine-2,6-dione (190 mg, 329.54 μmol, 30.90% yield, TFA) as a light yellow solid. MS(M+H)⁺=463.3

¹H NMR (400 MHz, DMSO-d₆) δ 10.98 (s, 1H), 8.18-8.05 (m, 3H), 7.66-7.55 (m, 2H), 7.54-7.45 (m, 3H), 7.37-7.31 (m, 2H), 5.30 (s, 2H), 5.16-5.08 (m, 1H), 4.49-4.39 (m, 1H), 4.33-4.17 (m, 3H), 3.29-3.15 (m, 2H), 3.08-2.86 (m, 3H), 2.65-2.56 (m, 1H), 2.44-2.39 (m, 1H), 2.15-1.96 (m, 4H), 1.79-1.66 (m, 2H).

### Step 9. Synthesis of (S)-4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-(((2-(2,6-dioxopiperidin-3-yl)-1-oxoisoindolin-4-yl)oxy)methyl)benzyl)piperidin-4-yl)-3-methoxybenzamide (Compound 21)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (60 mg, 134.10 μmol) in DMF (3 mL) were added HATU (120.00 mg, 315.60 μmol), DIPEA (148.40 mg, 1.15 mmol, 200 μL) and (S)-3-(4-((4-((4-aminopiperidin-1-yl)methyl)benzyl)oxy)-1-oxoisoindolin-2-yl)piperidine-2,6-dione (80 mg, 138.75 μmol, TFA) at 25° C. The mixture was stirred at 25° C. for 12 h under N₂ atmosphere. LCMS showed 45% peak with the desired mass was detected and no peak with the starting material. To the reaction mixture was added H₂O (10 mL), the mixture was extracted with EtOAc (30 mL×2), the combined organic layers were washed with brine (20 mL×3), dried over Na₂SO₄, filtered and the filtrate was concentrated under reduced pressure. The residue was purified by prep-HPLC (column: Phenomenex Synergi Polar-RP 100*25 mm*4 μm; mobile phase: [water (TFA)-ACN]; B %: 36%-56%, 7 min; Column Temp: 30° C.) and re-purified by prep-HPLC (column: Waters Xbridge 150*25 mm*5 μm; mobile phase: [water(NH₄HCO₃)-ACN]; B %: 46%-76%, 9 min; Column Temp: 30° C.) followed by lyophilization to afford (S)-4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-(((2-(2,6-dioxopiperidin-3-yl)-1-oxoisoindolin-4-yl)oxy)methyl)benzyl)piperidin-4-yl)-3-methoxybenzamide (28.1 mg, 29.93 μmol, 22.32% yield, 95% purity) as a white solid. MS(M+H)⁺=892.4

SFC indicated two peak (45.6%: 54.4%) and the product maybe was a racemization.

¹H NMR (400 MHz, DMSO-d₆) δ=10.97 (s, 1H), 8.31-8.23 (m, 2H), 8.15-8.08 (m, 1H), 7.96 (s, 1H), 7.52-7.42 (m, 5H), 7.37-7.30 (m, 4H), 5.24 (s, 2H), 5.15-5.08 (m, 1H), 4.82-4.71 (m, 1H), 4.47-4.38 (m, 1H), 4.31-4.20 (m, 1H), 4.08-4.00 (m, 2H), 3.94 (s, 3H), 3.84-3.73 (m, 1H), 3.49 (s, 2H), 3.30 (s, 3H), 2.93-2.81 (m, 3H), 2.63-2.58 (m, 1H), 2.09-1.91 (m, 6H), 1.84-1.69 (m, 3H), 1.66-1.51 (m, 7H).

### Example 22. (S)-4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-(((2-(2,6-dioxopiperidin-3-yl)-1-oxoisoindolin-5-yl)oxy)methyl)benzyl)piperidin-4-yl)-3-methoxybenzamide (Compound 22)

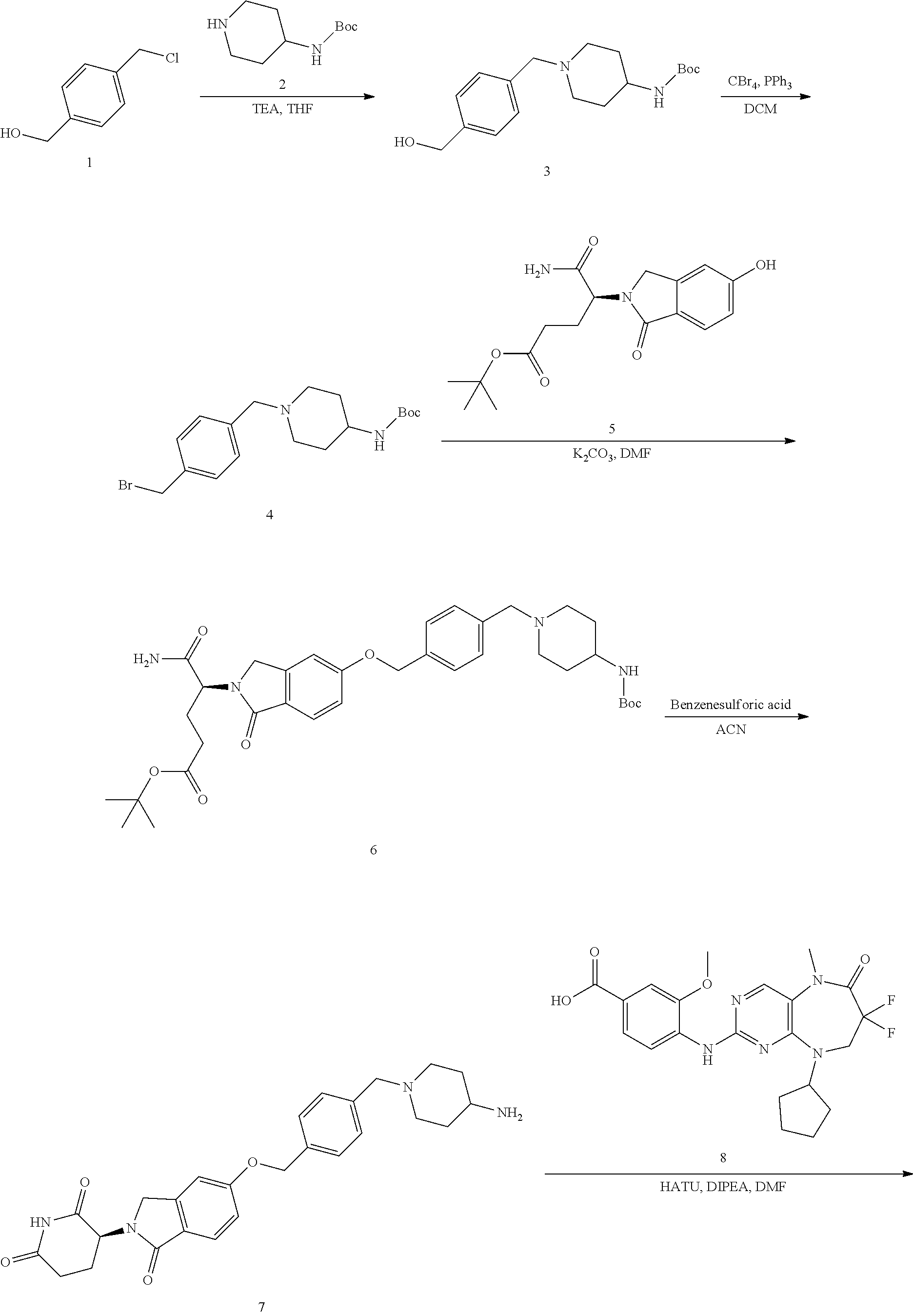

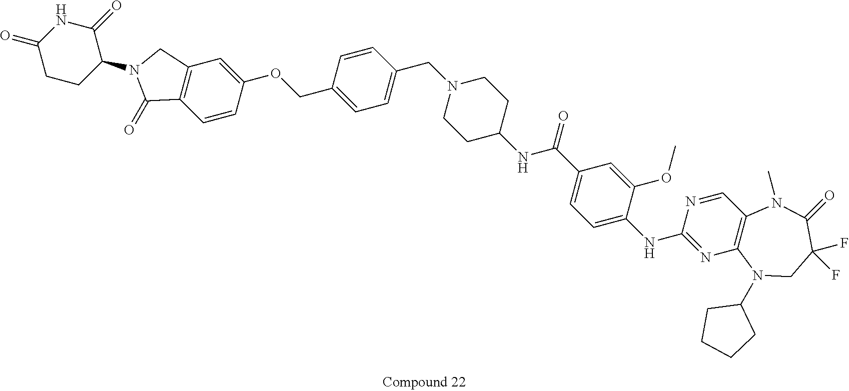 [SMILES: COc1cc(C(=O)NC2CCN(Cc3ccc(COc4ccc5c(c4)CN([C@H]4CCC(=O)NC4=O)C5=O)cc3)CC2)ccc1Nc1ncc2c(n1)N(C1CCCC1)CC(F)(F)C(=O)N2C]

### Step 1. Synthesis of tert-butyl (1-(4-(hydroxymethyl)benzyl)piperidin-4-yl)carbamate (3)

A mixture of (4-(chloromethyl)phenyl)methanol (2 g, 12.77 mmol), tert-butyl piperidin-4-ylcarbamate (2.56 g, 12.77 mmol) and TEA (2.58 g, 25.54 mmol, 3.56 mL) in THF (40 mL) was stirred at 25° C. for 4 hours. LCMS showed the starting material was consumed completely and a peak (58%) with desired mass. The reaction was concentrated in vacuum and purified by flash silica gel chromatography (20 g SepaFlash® Silica Flash Column, Eluent of 50˜100% EtOAc/Petroleum ether gradient @100 mL/min) to afford tert-butyl (1-(4-(hydroxymethyl)benzyl)piperidin-4-yl)carbamate (2.36 g, 7.37 mmol, 57.67% yield) as a white solid. MS(M+H)⁺=321.2

### Step 2. Synthesis of tert-butyl (1-(4-(bromomethyl)benzyl)piperidin-4-yl)carbamate (4)

To a solution of tert-butyl (1-(4-(hydroxymethyl)benzyl)piperidin-4-yl)carbamate (500 mg, 1.56 mmol) in DCM (20 mL) were added PPh₃ (613.92 mg, 2.34 mmol) and CBr₄ (776.22 mg, 2.34 mmol) at 0° C., the mixture was stirred at 25° C. for 12 hours. LCMS showed a peak (30%) with desired mass. The resulting mixture was concentrated in vacuum. The residue was purified by flash silica gel chromatography (12 g SepaFlash® Silica Flash Column, Eluent of 8˜19% EtOAc/Petroleum ether gradient @100 mL/min) to afford tert-butyl (1-(4-(bromomethyl)benzyl)piperidin-4-yl)carbamate (285 mg, 743.50 μmol, 47.65% yield) as a brown solid. MS(M+H)⁺=383.0

### Step 3. Synthesis of tert-butyl (S)-5-amino-4-(5-((4-((4-((tert-butoxycarbonyl)amino)piperidin-1-yl)methyl)benzyl)oxy)-1-oxoisoindolin-2-yl)-5-oxopentanoate (6)

To a solution of tert-butyl (S)-5-amino-4-(5-hydroxy-1-oxoisoindolin-2-yl)-5-oxopentanoate (250 mg, 747.68 μmol) and K₂CO₃ (206.67 mg, 1.50 mmol) in DMF (10 mL) was added a solution of tert-butyl (1-(4-(bromomethyl)benzyl)piperidin-4-yl)carbamate (285 mg, 743.50 μmol) in DMF (5 mL) dropwise at 0° C., the resulting mixture was stirred at 25° C. for 2 hours. LCMS showed 15% of tert-butyl (S)-5-amino-4-(5-hydroxy-1-oxoisoindolin-2-yl)-5-oxopentanoate remained and 45% of desired mass. The reaction mixture was diluted with H₂O (80 mL) and extracted with EtOAc (50 mL×4). The combined organic layers were washed with brine (100 mL×5), dried over Na₂SO₄, filtered and concentrated in vacuum. The residue was purified by pre-TLC (SiO₂, EtOAc:Methanol=10:1) followed by prep-TLC (SiO₂, EtOAc) to afford tert-butyl (S)-5-amino-4-(5-((4-((4-((tert-butoxycarbonyl)amino)piperidin-1-yl)methyl)benzyl)oxy)-1-oxoisoindolin-2-yl)-5-oxopentanoate (145 mg, 227.71 μmol, 30.46% yield) as an off-white solid. MS(M+H)⁺=637.2

### Step 4. Synthesis of (S)-3-(5-((4-((4-aminopiperidin-1-yl)methyl)benzyl)oxy)-1-oxoisoindolin-2-yl)piperidine-2,6-dione (7)

To a solution of tert-butyl (S)-5-amino-4-(5-((4-((4-((tert-butoxycarbonyl)amino)piperidin-1-yl)methyl)benzyl)oxy)-1-oxoisoindolin-2-yl)-5-oxopentanoate (110 mg, 172.75 μmol) in ACN (6 mL) was added benzenesulfonic acid (54.65 mg, 345.49 μmol), the mixture was stirred at 80° C. for 16 hours. LCMS showed the starting material was consumed completely and a peak (45%) with desired mass. The reaction mixture was concentrated in vacuum at 35° C. to afford (S)-3-(5-((4-((4-aminopiperidin-1-yl)methyl)benzyl)oxy)-1-oxoisoindolin-2-yl)piperidine-2,6-dione (85 mg) as a brown solid, which was used directly. MS(M+H)⁺=463.1

### Step 5. Synthesis of (S)-4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-(((2-(2,6-dioxopiperidin-3-yl)-1-oxoisoindolin-5-yl)oxy)methyl)benzyl)piperidin-4-yl)-3-methoxybenzamide (Compound 22)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (80 mg, 178.80 μmol) in DMF (3 mL) were added HATU (101.98 mg, 268.20 μmol) and DIPEA (138.65 mg, 1.07 mmol, 186.86 μL), the mixture was stirred at 15° C. for 10 minutes, then (S)-3-(5-((4-((4-aminopiperidin-1-yl)methyl)benzyl)oxy)-1-oxoisoindolin-2-yl)piperidine-2,6-dione (82.70 mg, 178.80 μmol) was added and the resulting mixture was stirred at 15° C. for 30 minutes. LCMS showed 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid was consumed completely and a peak (38%) with desired mass. To the mixture was added CH₃COOH to adjust pH<7 and purified by prep-HPLC (column: Phenomenex Synergi Polar-RP 100*25 mm*4 μm; mobile phase: [water(TFA)-ACN]; B %: 33%-53%, 7 min) followed by prep-HPLC (column: Shim-pack C18 150*25*10 μm; mobile phase: [water(TFA)-ACN]; B %: 18%-48%, 10 min). The impure product was re-purified by prep-HPLC (column: Phenomenex Luna C18 150*25 mm*10 μm; mobile phase: [water(FA)-ACN]; B %: 14%-44%, 10 min), the eluent was freeze-dried to afford (S)-4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-(((2-(2,6-dioxopiperidin-3-yl)-1-oxoisoindolin-5-yl)oxy)methyl)benzyl)piperidin-4-yl)-3-methoxybenzamide (7.9 mg, 8.68 μmol, 4.85% yield, 98% purity) as a white solid. MS(M+H)⁺=892.3

¹H NMR (400 MHz, DMSO-d₆) δ=10.97 (s, 1H), 8.31-8.24 (m, 2H), 8.12 (d, J=7.8 Hz, 1H), 7.96 (s, 1H), 7.64 (d, J=8.4 Hz, 1H), 7.53-7.46 (m, 2H), 7.45-7.40 (m, 2H), 7.34 (d, J=8.1 Hz, 2H), 7.26 (d, J=1.7 Hz, 1H), 7.13 (dd, J=2.1, 8.4 Hz, 1H), 5.19 (s, 2H), 5.07 (dd, J=5.0, 13.3 Hz, 1H), 4.84-4.69 (m, 1H), 4.47-4.22 (m, 2H), 4.04 (br t, J=14.1 Hz, 2H), 3.93 (s, 3H), 3.86-3.70 (m, 1H), 3.49 (s, 2H), 3.32 (s, 3H), 2.98-2.76 (m, 3H), 2.62-2.57 (m, 1H), 2.48-2.34 (m, 3H), 2.10-1.97 (m, 3H), 1.97-1.88 (m, 2H), 1.83-1.66 (m, 4H), 1.62-1.56 (m, 4H).

### Example 23. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-((2-(2,6-dioxopiperidin-3-yl)-1,3-dioxoisoindolin-4-yl)glycyl)piperidin-4-yl)-2-fluoro-5-methoxybenzamide (Compound 23)

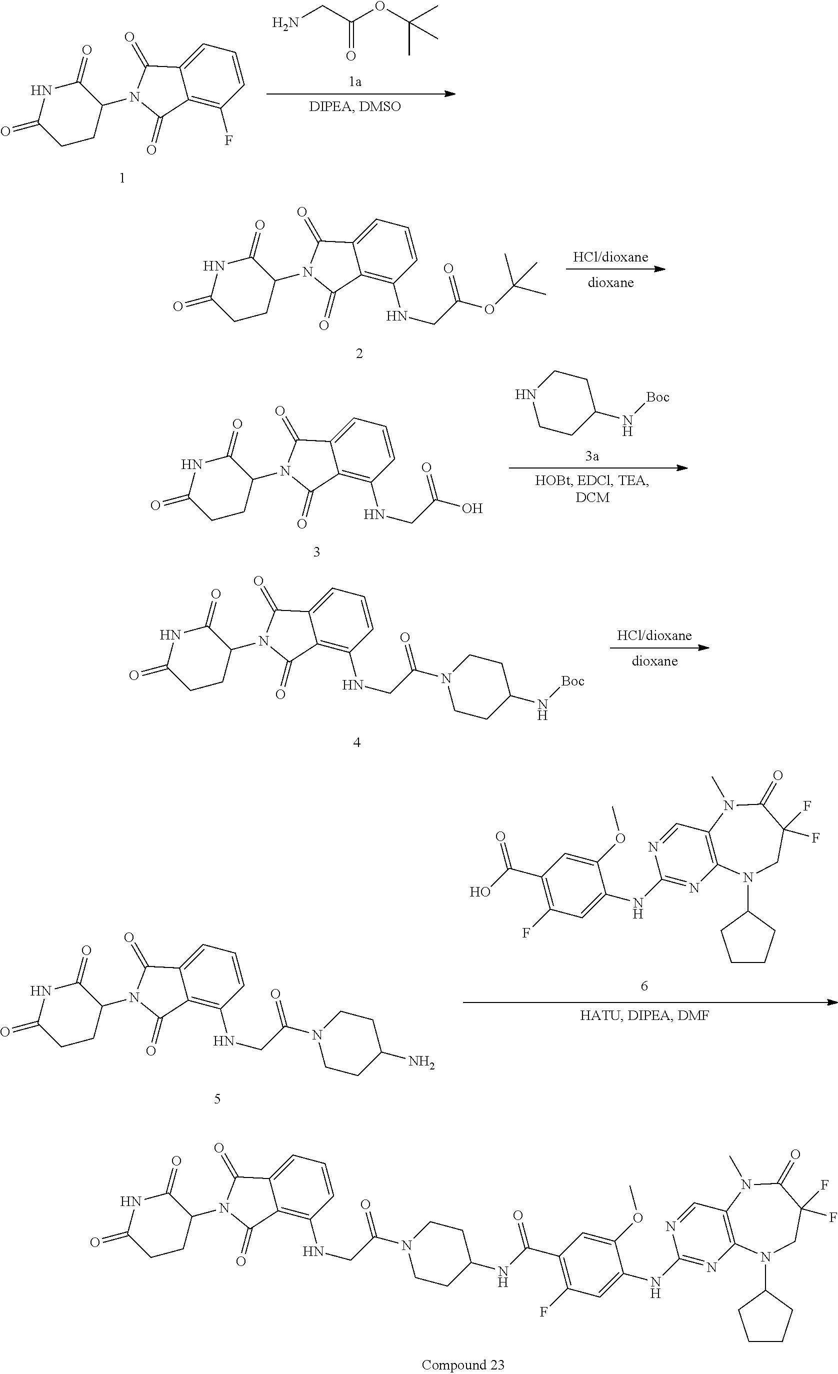

### Step 1. Synthesis of tert-butyl (2-(2,6-dioxopiperidin-3-yl)-1,3-dioxoisoindolin-4-yl)glycinate (2)

A solution of 2-(2,6-dioxopiperidin-3-yl)-4-fluoroisoindoline-1,3-dione (6 g, 21.72 mmol), tert-butyl glycinate (3.42 g, 26.06 mmol) and DIEA (5.61 g, 43.44 mmol, 7.57 mL) in DMSO (50 mL) was stirred at 90° C. for 24 hr. LCMS showed a peak (23%) with mass of 2-(2,6-dioxopiperidin-3-yl)-4-fluoroisoindoline-1,3-dione and a peak (43%) with desired mass. Another portion of tert-butyl glycinate (1.71 g, 13.03 mmol) was added to the reaction mixture at 25° C. The resulting mixture was stirred at 90° C. for another 12 hrs. LCMS showed the 2-(2,6-dioxopiperidin-3-yl)-4-fluoroisoindoline-1,3-dione was consumed completely and a peak (48%) with desired mass. The mixture was poured into water (150 mL) and extracted with EtOAc (30 mL×4). The combined organic phase was washed with brine (30 mL×3), dried over Na₂SO₄, filtered and concentrated under reduced pressure. The residue was purified by flash silica gel chromatography (45 g SepaFlash® Silica Flash Column, Eluent of 0˜50% EtOAc/Petroleum ether gradient @80 mL/min) to afford tert-butyl (2-(2,6-dioxopiperidin-3-yl)-1,3-dioxoisoindolin-4-yl)glycinate (1.8 g, crude) as yellow solid, which was used for the next step directly. MS(M−56)⁺=332.1

¹H NMR (400 MHz, CDCl₃) δ=8.24 (s, 1H), 7.51 (dd, J=7.2, 8.3 Hz, 1H), 7.15 (d, J=7.1 Hz, 1H), 6.76 (d, J=8.4 Hz, 1H), 4.99-4.88 (m, 1H), 3.95 (s, 2H), 2.97-2.63 (m, 3H), 2.18-2.08 (m, 1H), 1.50 (s, 9H).

### Step 2. Synthesis of (2-(2,6-dioxopiperidin-3-yl)-1,3-dioxoisoindolin-4-yl)glycine (3)

To a solution of tert-butyl (2-(2,6-dioxopiperidin-3-yl)-1,3-dioxoisoindolin-4-yl)glycinate (1.8 g, 4.65 mmol) in dioxane (5 mL) was added HCl/dioxane (4 M, 40 mL) at 25° C. The resulting mixture was stirred at 25° C. for 24 hr. LCMS showed the starting material was consumed completely and a main peak with desired mass. The mixture was concentrated under reduced pressure to afford (2-(2,6-dioxopiperidin-3-yl)-1,3-dioxoisoindolin-4-yl)glycine (1.5 g, crude) as yellow solid, which was used for the next step directly. MS(M+H)⁺=332.0

### Step 3. Synthesis of tert-butyl (1-((2-(2,6-dioxopiperidin-3-yl)-1,3-dioxoisoindolin-4-yl)glycyl)piperidin-4-yl)carbamate (4)

To a solution of (2-(2,6-dioxopiperidin-3-yl)-1,3-dioxoisoindolin-4-yl)glycine (500 mg, 1.51 mmol), HOBt (244.73 mg, 1.81 mmol), EDCI (347.20 mg, 1.81 mmol) and TEA (458.17 mg, 4.53 mmol, 630.22 μL) in DCM (5 mL) was added tert-butyl piperidin-4-ylcarbamate (392.96 mg, 1.96 mmol) at 25° C. The mixture was stirred at 25° C. for 12 h. LCMS showed the (2-(2,6-dioxopiperidin-3-yl)-1,3-dioxoisoindolin-4-yl)glycine was consumed completely and a peak (69%) with desired mass. The mixture was diluted with EtOAc (30 mL) and concentrated. The residue was purified by flash silica gel chromatography (12 g SepaFlash® Silica Flash Column, Eluent of 0˜80% EtOAc/Petroleum ether gradient @70 mL/min) to afford tert-butyl (1-((2-(2,6-dioxopiperidin-3-yl)-1,3-dioxoisoindolin-4-yl)glycyl)piperidin-4-yl)carbamate (0.6 g, 1.09 mmol, 71.99% yield, 93% purity) as yellow solid, which was used for the next step directly. MS(M−56)⁺=458.0

¹H NMR (400 MHz, CDCl₃) δ=8.28-8.13 (m, 1H), 7.50 (dd, J=7.3, 8.2 Hz, 1H), 7.13 (d, J=7.1 Hz, 1H), 6.79 (d, J=7.9 Hz, 1H), 4.94 (dd, J=5.4, 12.2 Hz, 1H), 4.65-4.49 (m, 2H), 4.03 (s, 2H), 3.79-3.63 (m, 2H), 3.26-3.12 (m, 1H), 2.94-2.73 (m, 4H), 2.18-2.06 (m, 2H), 1.99 (d, J=8.4 Hz, 2H), 1.46 (s, 9H).

### Step 4. Synthesis of 4-((2-(4-aminopiperidin-1-yl)-2-oxoethyl)amino)-2-(2,6-dioxopiperidin-3-yl)isoindoline-1,3-dione (5)

To a solution of tert-butyl (1-((2-(2,6-dioxopiperidin-3-yl)-1,3-dioxoisoindolin-4-yl)glycyl)piperidin-4-yl)carbamate (0.6 g, 1.17 mmol) in dioxane (2 mL) was added HCl/dioxane (4 M, 10 mL) at 25° C. The resulting mixture was stirred at 25° C. for 0.5 hr. LCMS showed the starting material was consumed completely and a main peak with desired mass. The mixture solution was concentrated under reduced pressure to afford 4-((2-(4-aminopiperidin-1-yl)-2-oxoethyl)amino)-2-(2,6-dioxopiperidin-3-yl)isoindoline-1,3-dione (0.4 g, crude, HCl) as yellow solid, which was used for the next step directly. MS (M+H)⁺=414.0

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-((2-(2,6-dioxopiperidin-3-yl)-1,3-dioxoisoindolin-4-yl)glycyl)piperidin-4-yl)-2-fluoro-5-methoxybenzamide (Compound 23)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (60 mg, 128.91 μmol, 1 eq) in DMF (1 mL) was added HATU (73.53 mg, 193.37 μmol, 1.5 eq) and DIPEA (49.98 mg, 386.74 μmol, 67.36 μL). The mixture was stirred at 30° C. for 10 min. To mixture was added 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (58.00 mg, 128.91 μmol, HCl). The mixture was stirred at 30° C. for 12 h. LCMS showed the 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid was consumed completely and a main peak with desired mass. The mixture was purified by prep-HPLC (column: Phenomenex Synergi Polar-RP 100×25 mm×4 um; mobile phase: [water(TFA)-ACN]; B %: 48%-68%, 7 min) and re-purified by prep-HPLC (column: Waters Xbridge 150×25 mm×5 μm; mobile phase: [water(NH₄HCO₃)-ACN]; B %: 42%-72%, min) and lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-((2-(2,6-dioxopiperidin-3-yl)-1,3-dioxoisoindolin-4-yl) glycyl)piperidin-4-yl)-2-fluoro-5-methoxybenzamide (18 mg, 20.07 μmol, 15.57% yield, 96% purity) as yellow solid. MS(M+H)⁺=861.3

¹H NMR (400 MHz, DMSO-d₆) δ=11.33-10.80 (m, 1H), 8.33-8.21 (m, 2H), 8.09-7.98 (m, 2H), 7.61 (t, J=7.8 Hz, 1H), 7.20 (d, J=6.7 Hz, 1H), 7.16-7.03 (m, 3H), 5.07 (dd, J=5.4, 12.9 Hz, 1H), 4.91-4.75 (m, 1H), 4.39-4.02 (m, 6H), 3.98-3.86 (m, 4H), 3.33-3.32 (m, 3H), 3.20 (t, J=11.6 Hz, 1H), 2.95-2.80 (m, 2H), 2.63-2.55 (m, 2H), 2.06-1.84 (m, 5H), 1.75-1.41 (m, 8H).

### Example 24. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-((2-(2,6-dioxopiperidin-3-yl)-1,3-dioxoisoindolin-4-yl)amino)propanoyl)piperidin-4-yl)-2-fluoro-5-methoxybenzamide (Compound 24)

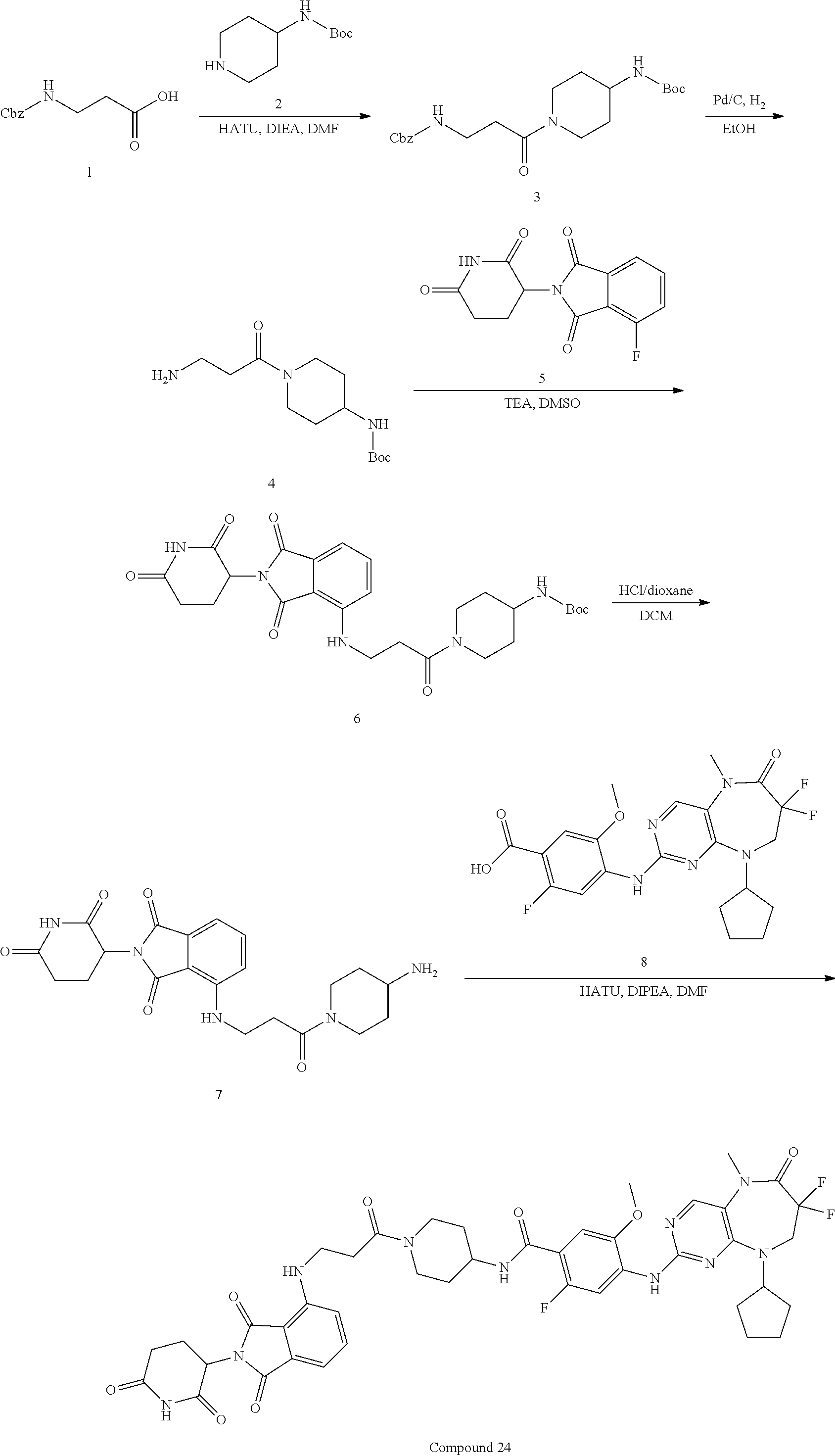

### Step 1. Synthesis of benzyl (3-(4-((tert-butoxycarbonyl)amino)piperidin-1-yl)-3-oxopropyl) carbamate (3)

To a solution of 3-(((benzyloxy)carbonyl)amino)propanoic acid (0.5 g, 2.24 mmol) and tert-butyl piperidin-4-ylcarbamate (448.60 mg, 2.24 mmol) in DMF (7 mL) were added HATU (1.28 g, 3.36 mmol) and DIEA (868.47 mg, 6.72 mmol, 1.17 mL). The mixture was stirred at 25 C for 16 h. LCMS showed a peak (68%) with desired mass. The reaction mixture was diluted with water (100 mL) and extracted with EtOAc (30 mL×3). The combined organic layers were washed with brine (30 mL), dried over anhydrous sodium sulfate, filtered and concentrated under reduced pressure. The residue was purified by column chromatography (SiO₂, Petroleum ether/EtOAc=1/0 to 1/1) to afford benzyl (3-(4-((tert-butoxycarbonyl)amino)piperidin-1-yl)-3-oxopropyl)carbamate (800 mg, 1.95 mmol, 87.20% yield, 99% purity) as a black brown solid. MS(M+H)⁺=406.2

### Step 2. Synthesis of tert-butyl (1-(3-aminopropanoyl)piperidin-4-yl)carbamate (4)

To a solution of benzyl (3-(4-((tert-butoxycarbonyl)amino)piperidin-1-yl)-3-oxopropyl)carbamate (0.8 g, 1.97 mmol) in EtOH (10 mL) was added Pd/C (0.1 g, 10% purity) under N₂ atmosphere. The mixture was stirred at 25° C. for 16 h under H₂ (15 psi). LCMS showed the starting material was consumed completely and a peak (73%) with desired mass. The reaction mixture was filtered and filter cake was washed with EtOH (100 mL). The filtrate was concentrated in vacuo to afford tert-butyl (1-(3-aminopropanoyl)piperidin-4-yl)carbamate (480 mg, 1.29 mmol, 65.45% yield, 73% purity) as colorless oil. MS(M+H)⁺=272.2

### Step 3. Synthesis of tert-butyl (1-(3-((2-(2,6-dioxopiperidin-3-yl)-1,3-dioxoisoindolin-4-yl)amino)propanoyl)piperidin-4-yl)carbamate (6)

To a solution of tert-butyl (1-(3-aminopropanoyl)piperidin-4-yl)carbamate (450 mg, 1.66 mmol) in DMSO (7 mL) were added TEA (503.42 mg, 4.98 mmol, 692.46 μL) and 2-(2,6-dioxopiperidin-3-yl)-4-fluoroisoindoline-1,3-dione (458.07 mg, 1.66 mmol). The mixture was stirred at 80° C. for 1 h. LCMS showed ˜44% of tert-butyl (1-(3-aminopropanoyl)piperidin-4-yl)carbamate remained and a peak (˜37%) with desired mass. The reaction mixture was diluted with water (100 mL) and extracted with EtOAc (30 mL×3). The combined organic layers were washed with brine (30 mL), dried over anhydrous sodium sulfate, filtered and concentrated under reduced pressure. The residue was purified by column chromatography (SiO₂, Petroleum ether/EtOAc=1/0 to 0/1) to afford tert-butyl (1-(3-((2-(2,6-dioxopiperidin-3-yl)-1,3-dioxoisoindolin-4-yl)amino)propanoyl)piperidin-4-yl)carbamate. MS(M−56+H)⁺=472.0

### Step 4. Synthesis of 4-((3-(4-aminopiperidin-1-yl)-3-oxopropyl)amino)-2-(2,6-dioxopiperidin-3-yl)isoindoline-1,3-dione (7)

A mixture of tert-butyl (1-(3-((2-(2,6-dioxopiperidin-3-yl)-1,3-dioxoisoindolin-4-yl)amino)propanoyl)piperidin-4-yl)carbamate (100 mg, 189.55 μmol), HCl/dioxane (2 M, 1 mL) in DCM (2 mL) was stirred at 25° C. for 0.5 h. LCMS showed the starting material was consumed completely and one main peak with desired mass was detected. The reaction mixture was concentrated under reduced pressure to afford 4-((3-(4-aminopiperidin-1-yl)-3-oxopropyl)amino)-2-(2,6-dioxopiperidin-3-yl)isoindoline-1,3-dione (100 mg, crude, HCl salt) as a yellow solid. MS(M+H)⁺=428.1

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-((2-(2,6-dioxopiperidin-3-yl)-1,3-dioxoisoindolin-4-yl)amino)propanoyl)piperidin-4-yl)-2-fluoro-5-methoxybenzamide (Compound 24)

To a solution of 4-((3-(4-aminopiperidin-1-yl)-3-oxopropyl)amino)-2-(2,6-dioxopiperidin-3-yl)isoindoline-1,3-dione (100 mg, 233.94 μmol, HCl salt) and 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (108.88 mg, 233.94 μmol) in DMF (3 mL) were added HATU (133.43 mg, 350.92 μmol) and DIEA (90.71 mg, 701.83 μmol, 122.25 μL). The mixture was stirred at 25° C. for 5 h. LCMS showed 4-((3-(4-aminopiperidin-1-yl)-3-oxopropyl)amino)-2-(2,6-dioxopiperidin-3-yl)isoindoline-1,3-dione was consumed completely and one main peak with desired mass was detected. The reaction mixture was diluted with water (50 mL) and extracted with EtOAc (20 mL×3). The combined organic layers were washed with brine (20 mL), dried over anhydrous sodium sulfate, filtered and concentrated under reduced pressure. The residue was purified by prep-HPLC (Instrument: ACSWH-GX-K; Column: Phenomenex Gemini-NX C18 75×30 mm×3 μm; eluent A: 0.225% formic acid in water, eluent B: acetonitrile; gradient: 7 min 42-72% B; flow 25 ml/min; temperature: room temperature; Detector: UV 220/254 nm) to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-((2-(2,6-dioxopiperidin-3-yl)-1,3-dioxoisoindolin-4-yl)amino)propanoyl)piperidin-4-yl)-2-fluoro-5-methoxybenzamide (40.6 mg, 45.48 umol, 19.44% yield, 98% purity) as a yellow solid. MS(M+H)⁺=875.0

¹H NMR (400 MHz, DMSO-d₆) δ=11.10 (s, 1H), 8.31 (s, 1H), 8.25 (d, J=13.3 Hz, 1H), 8.09 (s, 1H), 7.97-7.95 (m, 7.7 Hz, 1H), 7.60 (dd, J=7.1, 8.5 Hz, 1H), 7.21 (d, J=6.8 Hz, 1H), 7.16 (d, J=8.6 Hz, 1H), 7.04 (d, J=7.0 Hz, 1H), 6.80-6.75 (m, 1H), 5.05 (dd, J=5.4, 12.9 Hz, 1H), 4.87-4.79 (m, 1H), 4.35-4.31 (m, 1H), 4.09-4.05 (m, 2H), 4.04-3.98 (m, 1H), 3.92 (s, 3H), 3.90-3.81 (m, 1H), 3.56-3.52 (m, 2H), 3.34 (s, 3H), 3.20-3.13 (m, 1H), 2.93-2.74 (m, 2H), 2.70-2.65 (m, 2H), 2.63-2.55 (m, 2H), 2.05-1.94 (m, 3H), 1.89-1.81 (m, 2H), 1.80-1.70 (m, 2H), 1.68-1.58 (m, 4H), 1.52-1.35 (m, 2H).

### Example 25. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1,3-dioxoisoindolin-4-yl)amino)butanoyl)piperidin-4-yl)-3-methoxybenzamide (Compound 25)

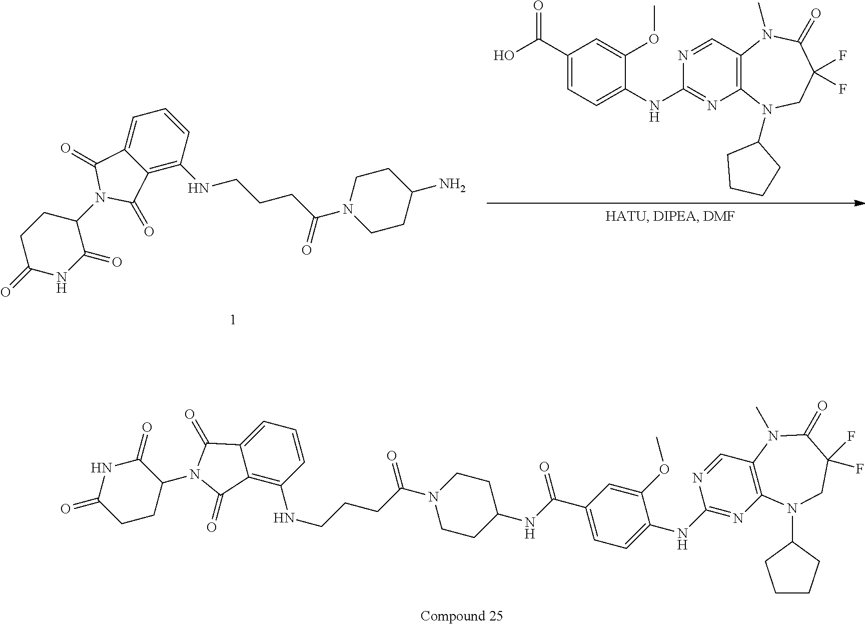 [SMILES: NC1CCN(C(=O)CCCNc2cccc3c2C(=O)N(C2CCC(=O)NC2=O)C3=O)CC1 ; COc1cc(C(=O)O)ccc1Nc1ncc2c(n1)N(C1CCCC1)CC(F)(F)C(=O)N2C ; "HATU, DIPEA, DMF" ; COc1cc(C(=O)NC2CCN(C(=O)CCCNc3cccc4c3C(=O)N(C3CCC(=O)NC3=O)C4=O)CC2)ccc1Nc1ncc2c(n1)N(C1CCCC1)CC(F)(F)C(=O)N2C]

The compound 25 was synthesized by the method described in the scheme similar to the method described in Example 24.

MS(M+H)⁺=871.3, ¹H NMR (400 MHz, DMSO-d₆) δ=8.30-8.25 (m, 2H), 8.19-8.09 (m, 1H), 7.97 (s, 1H), 7.60 (t, J=7.7 Hz, 1H), 7.51-7.45 (m, 2H), 7.19 (d, J=8.8 Hz, 1H), 7.03 (d, J=7.1 Hz, 1H), 6.69 (t, J=5.9 Hz, 1H), 5.09-5.01 (m, 1H), 4.82-4.72 (m, 1H), 4.45-4.37 (m, 1H), 4.08-4.00 (m, 3H), 3.93 (s, 3H), 3.91-3.85 (m, 1H), 3.32-3.28 (m, 5H), 3.18-3.06 (m, 1H), 2.95-2.81 (m, 1H), 2.62-2.58 (m, 2H), 2.45-2.40 (m, 3H), 2.06-1.99 (m, 1H), 1.98-1.91 (m, 2H), 1.86-1.77 (m, 4H), 1.74-1.67 (m, 2H), 1.63-1.55 (m, 4H), 1.49-1.36 (m, 2H).

### Example 26. 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1-oxoisoindolin-4-yl)amino)butanoyl)piperidin-4-yl)-3-methoxybenzamide (Compound 26)

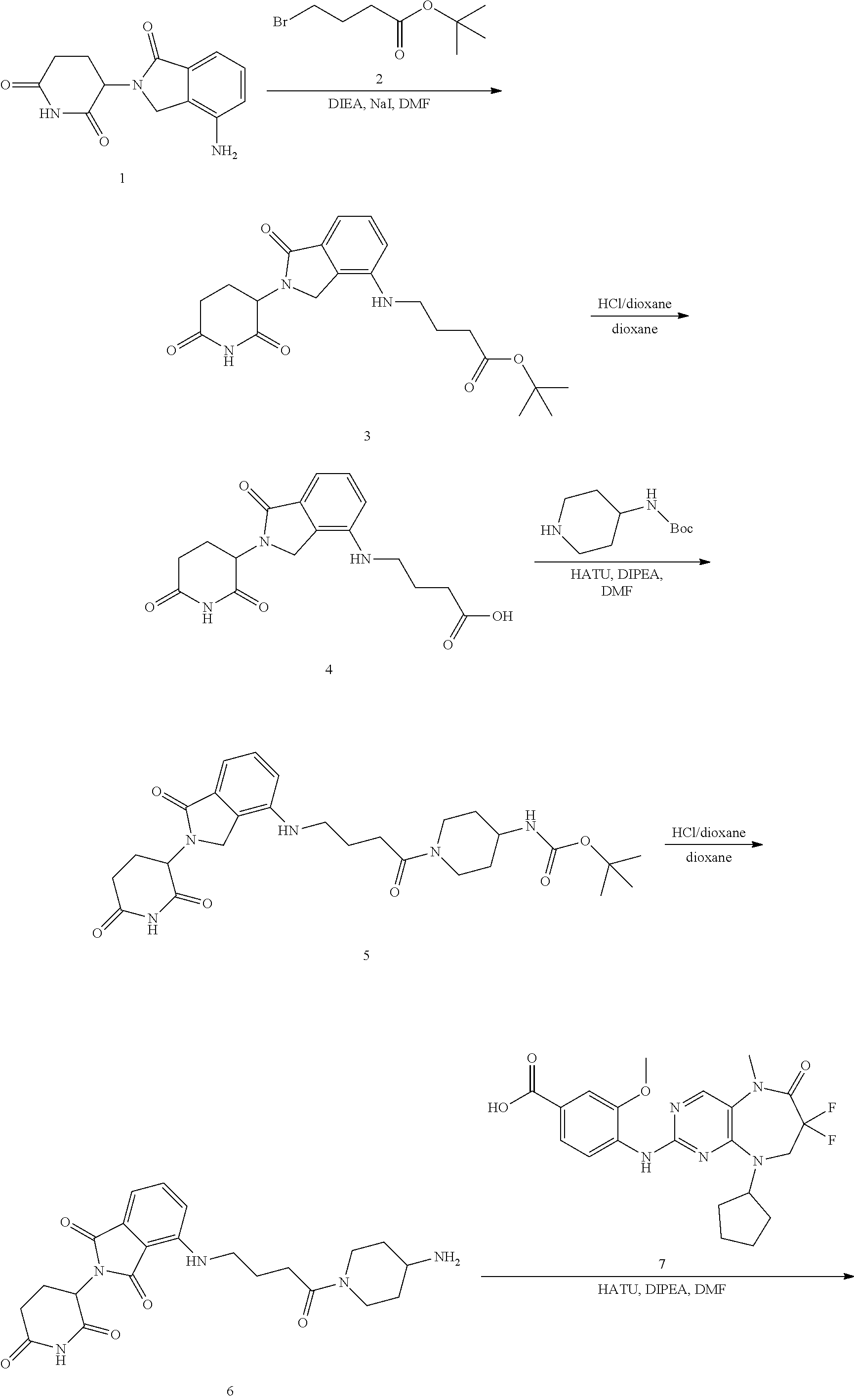

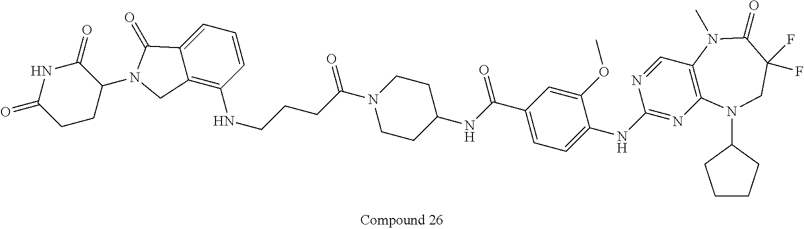 [SMILES: COc1cc(C(=O)NC2CCN(C(=O)CCCNc3cccc4c3CN(C3CCC(=O)NC3=O)C4=O)CC2)ccc1Nc1ncc2c(n1)N(C1CCCC1)CC(F)(F)C(=O)N2C]

### Step 1. Synthesis of tert-butyl 4-((2-(2,6-dioxopiperidin-3-yl)-1-oxoisoindolin-4-yl)amino)butanoate (3)

To the solution of 3-(4-amino-1-oxoisoindolin-2-yl)piperidine-2,6-dione (1 g, 3.86 mmol) and tert-butyl 4-bromobutanoate (1.03 g, 4.63 mmol) in DMF (10 mL) was added DIPEA (997.01 mg, 7.71 mmol, 1.34 mL) and NaI (57.82 mg, 385.71 μmol) and the resulting mixture was stirred at 100° C. for 12 h. LCMS showed that the reaction was completed, the mixture was poured into water (100 mL) and extracted with EtOAc (50 mL×3), the combined organic layer was washed with brine (100 mL), dried over Na₂SO₄, filtered and concentrated. The residue was purified by flash silica gel chromatography (ISCO®; 20 g SepaFlash® Silica Flash Column, Eluent of 0˜50% EtOAc/Petroleum ether gradient @80 mL/min) to afford tert-butyl 4-((2-(2,6-dioxopiperidin-3-yl)-1-oxoisoindolin-4-yl)amino)butanoate (390 mg, 0.689 mmol, 17.88% yield, 71% purity) as yellow solid. MS(M+H)⁺=402.2

### Step 2. Synthesis of 4-((2-(2,6-dioxopiperidin-3-yl)-1-oxoisoindolin-4-yl)amino)butanoic acid (4)

To the solution of tert-butyl 4-((2-(2,6-dioxopiperidin-3-yl)-1-oxoisoindolin-4-yl)amino)butanoate (390 mg, 971.47 μmol) in dioxane (5 mL) was added HCl/dioxane (4 M, 5 mL) and the resulting mixture was stirred at 20° C. for 12 h. LCMS showed that reaction was completed, the mixture was concentrated to afford 4-((2-(2,6-dioxopiperidin-3-yl)-1-oxoisoindolin-4-yl)amino)butanoic acid (350 mg, 0.917 mmol, 94.36% yield, HCl) as yellow solid. MS(M+H)⁺=346.0

### Step 3. Synthesis of tert-butyl (1-(4-((2-(2,6-dioxopiperidin-3-yl)-1-oxoisoindolin-4-yl)amino)butanoyl)piperidin-4-yl)carbamate (5)

To the solution of 4-((2-(2,6-dioxopiperidin-3-yl)-1-oxoisoindolin-4-yl)amino)butanoic acid (150 mg, 392.87 μmol HCl) and tert-butyl N-(4-piperidyl) carbamate (78.68 mg, 392.87 μmol) in DMF (2 mL) was added HATU (224.07 mg, 589.30 μmol) and DIPEA (203.10 mg, 1.57 mmol, 273.72 μL) and the resulting mixture was stirred at 20° C. for 12 h. LCMS showed that the reaction was completed, the mixture was adjusted pH=7 by FA and concentrated, the residue was purified by prep-HPLC (column: Unisil 3-100 CIs Ultra 150*50 mm*3 μm; mobile phase: [water (0.225% FA)-ACN]; B %: 26%-56%, 10 min) and the eluant was concentrated to afford tert-butyl (1-(4-((2-(2,6-dioxopiperidin-3-yl)-1-oxoisoindolin-4-yl)amino)butanoyl)piperidin-4-yl)carbamate (130 mg, 229.15 umol, 58.33% yield, 93% purity) as yellow oil. MS(M+H)⁺=528.2

### Step 4. Synthesis of 4-((4-(4-aminopiperidin-1-yl)-4-oxobutyl)amino)-2-(6-oxopiperidin-3-yl)isoindoline-1,3-dione (6)

To the solution of tert-butyl (1-(4-((2-(2,6-dioxopiperidin-3-yl)-1-oxoisoindolin-4-yl)amino)butanoyl)piperidin-4-yl)carbamate (130 mg, 246.39 μmol) in dioxane (2 mL) was added HCl/dioxane (4 M, 2 mL) and the resulting mixture was stirred at 20° C. for 12 h. LCMS showed that the reaction was completed, the mixture was concentrated to afford 4-((4-(4-aminopiperidin-1-yl)-4-oxobutyl)amino)-2-(6-oxopiperidin-3-yl)isoindoline-1,3-dione (0.1 g, 233.92 μmol, 94.94% yield) as yellow solid. MS(M+H)⁺=428.2

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1-oxoisoindolin-4-yl)amino)butanoyl)piperidin-4-yl)-3-methoxybenzamide (Compound 26)

To the solution of 4-((4-(4-aminopiperidin-1-yl)-4-oxobutyl)amino)-2-(6-oxopiperidin-3-yl)isoindoline-1,3-dione (60 mg, 129.32 μmol, HCl) and 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (57.86 mg, 129.32 μmol) in DMF (2 mL) was added HATU (73.76 mg, 193.98 μmol) and DIPEA (66.86 mg, 517.29 μmol, 90.10 μL) and the resulting mixture was stirred at 20° C. for 2 h. LCMS showed that the reaction was completed, the mixture was poured into water (20 mL) and extracted with EtOAc (20 mL×3), the combined organic layer was washed with brine (50 mL), dried over Na₂SO₄, filtered and concentrated. The residue was purified by prep-HPLC (column: Waters Xbridge 150*25 mm*5 μm; mobile phase: [water (10 mM NH₄HCO₃)-ACN]; B %: 32%-62%, 2 min) and re-purified by prep-TLC (EtOAc/Methanol=10/1) to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1-oxoisoindolin-4-yl)amino)butanoyl)piperidin-4-yl)-3-methoxybenzamide (15 mg, 15.58 μmol, 12.05% yield, 89% purity) as white solid. MS(M+H)⁺=857.5

¹H NMR (400 MHz, DMSO-d₆) δ=11.00 (s, 1H), 8.30-8.23 (m, 2H), 8.13 (d, J=7.9 Hz, 1H), 7.97 (s, 1H), 7.49-7.45 (m, 2H), 7.29 (t, J=7.6 Hz, 1H), 6.95-6.90 (m, 1H), 6.80 (d, J=8.1 Hz, 1H), 5.68 (t, J=5.7 Hz, 1H), 5.11 (dd, J=5.4, 13.4 Hz, 1H), 4.75 (d, J=8.3 Hz, 1H), 4.41 (d, J=13.9 Hz, 1H), 4.27-4.19 (m, 1H), 4.16-4.10 (m, 1H), 4.04 (t, J=14.3 Hz, 3H), 3.93 (s, 3H), 3.31-3.29 (m, 3H), 3.20-3.09 (m, 2H), 2.98-2.85 (m, 1H), 2.71-2.58 (m, 2H), 2.45-2.40 (m, 4H), 2.35-2.32 (m, 1H), 2.06-2.00 (m, 1H), 1.95-1.92 (m, 2H), 1.89-1.78 (m, 4H), 1.73-1.68 (m, 2H), 1.61-1.56 (m, 4H), 1.52-1.33 (m, 2H).

### Example 27. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1,3-dioxoisoindolin-4-yl)amino)butanoyl)piperidin-4-yl)-3-fluorobenzamide (Compound 27)

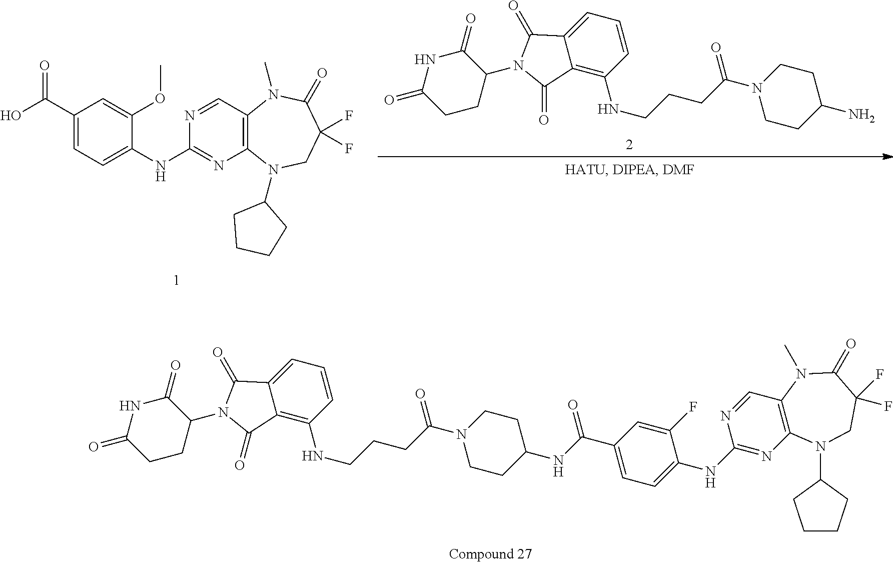

The compound 27 was synthesized by the method described in the scheme similar to the method described in Example 26.

MS(M+H)⁺=859.0, ¹H NMR (400 MHz, DMSO-d₆) δ=11.21-10.93 (m, 1H), 9.09 (s, 1H), 8.26-8.19 (m, 2H), 8.00 (t, J=8.3 Hz, 1H), 7.74-7.64 (m, 2H), 7.62-7.57 (m, 1H), 7.19 (d, J=8.7 Hz, 1H), 7.02 (d, J=7.0 Hz, 1H), 6.69 (br t, J=6.1 Hz, 1H), 5.05 (dd, J=5.3, 12.9 Hz, 1H), 4.73-4.63 (m, 1H), 4.38 (br d, J=13.3 Hz, 1H), 4.01 (br t, J=14.0 Hz, 3H), 3.89 (br d, J=13.2 Hz, 1H), 3.32 (s, 5H), 3.11 (br t, J=12.2 Hz, 1H), 2.95-2.82 (m, 1H), 2.68 (br d, J=9.9 Hz, 1H), 2.62-2.54 (m, 2H), 2.43 (br t, J=7.1 Hz, 2H), 2.07-1.97 (m, 1H), 1.91-1.75 (m, 6H), 1.72-1.61 (m, 2H), 1.60-1.32 (m, 6H).

### Example 28. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1,3-dioxoisoindolin-4-yl)amino)butanoyl)piperidin-4-yl)-3-(trifluoromethoxy)benzamide (Compound 28)

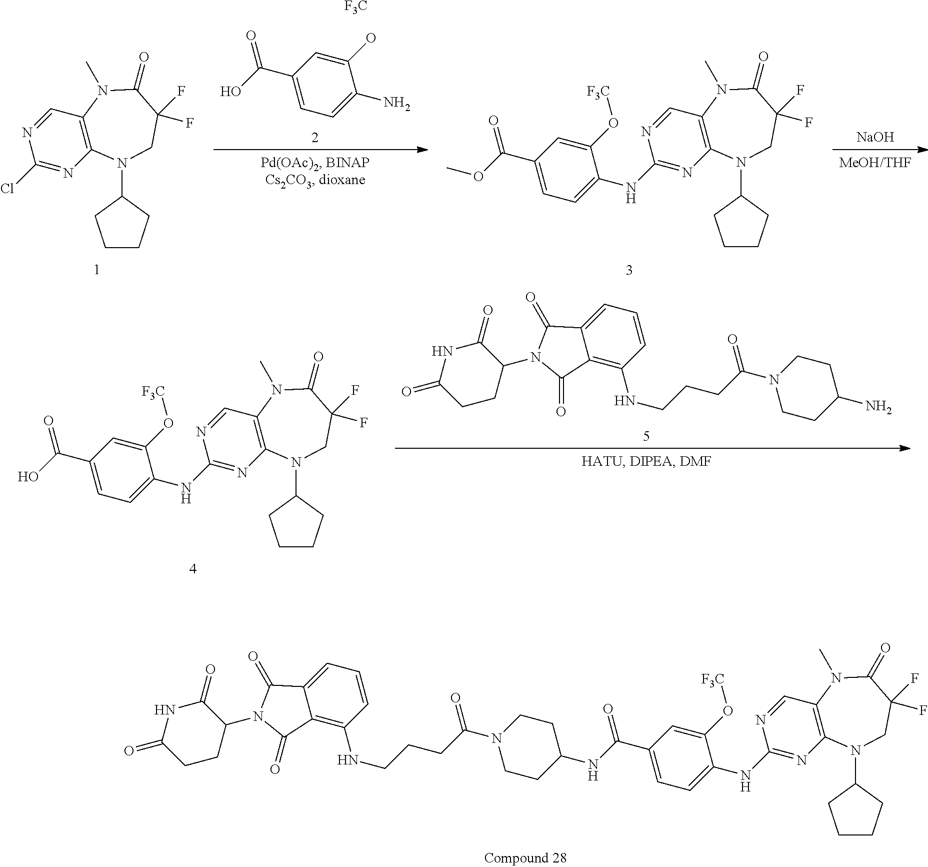

### Step 1. Synthesis of methyl 4-((9-cyclopentyl-7,7-difluoro-5-methyl-6-oxo-6,7,8,9-tetrahydro-5H-pyrimido[4,5-b][1,4]diazepin-2-yl)amino)-3-(trifluoromethoxy)benzoate (3)

To a solution of 2-chloro-9-cyclopentyl-7,7-difluoro-5-methyl-5,7,8,9-tetrahydro-6H-pyrimido[4,5-b][1,4]diazepin-6-one (200 mg, 631.45 μmol) and methyl 4-amino-3-(trifluoromethoxy) benzoate (178.19 mg, 757.73 μmol) in dioxane (10 mL) were added Pd(OAc)₂ (14.18 mg, 63.14 μmol,), BINAP (39.32 mg, 63.14 μmol) and Cs₂CO₃ (617.21 mg, 1.89 mmol) at 20° C. under N₂ atmosphere and the resulting mixture was stirred at 100° C. for 16 h under N₂ atmosphere. LCMS showed the starting material was consumed completely and a peak (65%) with desired mass. The reaction mixture was concentrated in vacuum. The residue was purified by flash silica gel chromatography (10 g SepaFlash® Silica Flash Column, Eluent of 0˜15% EtOAc/Petroleum ether gradient @100 ml/min) to afford methyl 4-((9-cyclopentyl-7,7-difluoro-5-methyl-6-oxo-6,7,8,9-tetrahydro-5H-pyrimido[4,5-b][1,4]diazepin-2-yl)amino)-3-(trifluoromethoxy)benzoate (316 mg, 588.55 μmol, 93.21% yield, 96% purity) as a white solid. MS(M+H)⁺=516.1

### Step 2. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-3-(trifluoromethoxy)benzoic acid (4)

To a solution of methyl 4-((9-cyclopentyl-7,7-difluoro-5-methyl-6-oxo-6,7,8,9-tetrahydro-5H-pyrimido[4,5-b][1,4]diazepin-2-yl)amino)-3-(trifluoromethoxy)benzoate (316 mg, 613.08 μmol) in THF (6 mL) and MeOH (6 mL) was added NaOH (2 M, 3 mL) at 20° C. and the resulting mixture was stirred at 20° C. for 16 h. LCMS showed the starting material was consumed completely and a peak (35%) with desired mass. The reaction mixture was diluted with H₂O (10 mL), then HCl (12 N) was added to adjust the PH<3. The suspension was concentrated in vacuum. The crude product was triturated with a mixture solvent (MTBE/EtOAc (4 mL/4 mL)) at 20° C. for 0.5 h, then filtered. The filtrate was concentrated in vacuum to afford 4-((9-cyclopentyl-7,7-difluoro-5-methyl-6-oxo-6,7,8,9-tetrahydro-5H-pyrimido[4,5-b][1,4]diazepin-2-yl)amino)-3-(trifluoromethoxy)benzoic acid (256 mg, 500.35 μmol, 81.61% yield, 98% purity) as an off-white solid. MS(M+H)⁺=502.2

### Step 3. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1,3-dioxoisoindolin-4-yl)amino)butanoyl)piperidin-4-yl)-3-(trifluoromethoxy)benzamide (Compound 28)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-(trifluoromethoxy)benzoic acid (80 mg, 159.55 μmol) in DMF (2 mL) were added HATU (66.73 mg, 175.51 μmol) and DIPEA (41.24 mg, 319.10 μmol, 55.58 μL), after stirring at 20° C. for 10 min and a solution of 4-((4-(4-aminopiperidin-1-yl)-4-oxobutyl)amino)-2-(2,6-dioxopiperidin-3-yl)isoindoline-1,3-dione (77.48 mg, 175.51 μmol) in DMF (2 mL) with DIPEA (41.24 mg, 319.10 μmol, 55.58 μL) was added and the resulting mixture was stirred at 20° C. for 1 h. LCMS showed starting material was consumed completely and a peak (31%) with desired mass. The reaction mixture was diluted with H₂O (12 mL) and extracted with EtOAc (12 mL×3). The organic layer was dried over Na₂SO₄, filtered and concentrated. The residue was purified by prep-HPLC (column: Waters Xbridge 150*25 mm*5 μm; mobile phase: [water(NH₄HCO₃)-ACN]; B %: 50%-80%, 7 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1,3-dioxoisoindolin-4-yl)amino)butanoyl)piperidin-4-yl)-3-(trifluoromethoxy)benzamide (14.2 mg, 14.43 μmol, 9.05% yield, 94% purity) as a yellow solid. MS(M+H)⁺=925.0

¹H NMR (400 MHz, DMSO-d₆) δ=11.22-10.95 (m, 1H), 9.01 (s, 1H), 8.34 (br d, J=7.6 Hz, 1H), 8.24 (s, 1H), 8.15 (d, J=9.1 Hz, 1H), 7.89-7.81 (m, 2H), 7.60 (dd, J=7.3, 8.4 Hz, 1H), 7.19 (d, J=8.6 Hz, 1H), 7.02 (d, J=7.0 Hz, 1H), 6.69 (br t, J=6.1 Hz, 1H), 5.05 (dd, J=5.4, 12.8 Hz, 1H), 4.73-4.62 (m, 1H), 4.39 (br d, J=12.6 Hz, 1H), 4.08-3.98 (m, 3H), 3.89 (br d, J=13.1 Hz, 1H), 3.34-3.30 (m, 5H), 3.16-3.05 (m, 1H), 2.93-2.83 (m, 1H), 2.73-2.65 (m, 1H), 2.62-2.53 (m, 2H), 2.43 (br t, J=7.0 Hz, 2H), 2.06-1.97 (m, 1H), 1.89-1.77 (m, 6H), 1.72-1.63 (m, 2H), 1.60-1.45 (m, 5H), 1.42-1.32 (m, 1H).

### Example 29. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(4-((2-(2,6-dioxopiperidin-3-yl)-1,3-dioxoisoindolin-4-yl)amino)butanoyl)piperazin-1-yl)-2-fluoro-5-methoxybenzamide (Compound 29)

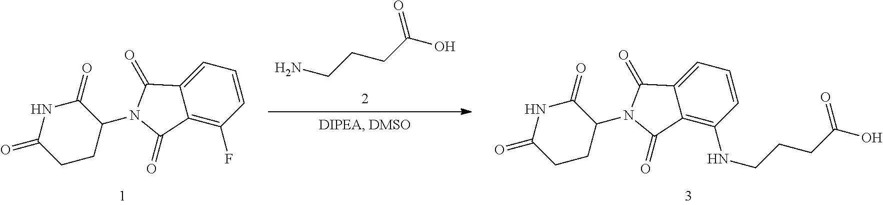

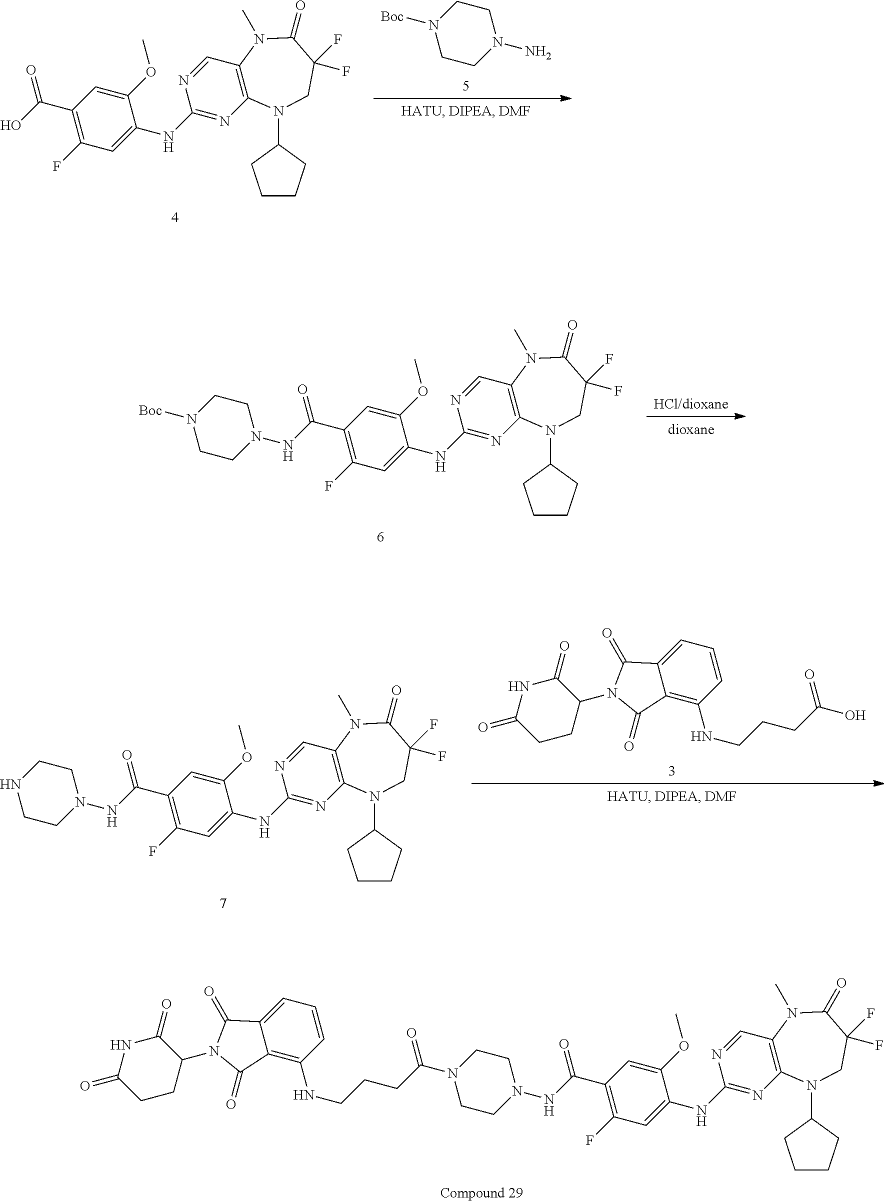

### Step 1. Synthesis of 4-((2-(2,6-dioxopiperidin-3-yl)-1,3-dioxoisoindolin-4-yl)amino) butanoic acid (3)

To a solution of 2-(2,6-dioxopiperidin-3-yl)-4-fluoroisoindoline-1,3-dione (1 g, 3.62 mmol, 1 eq) and 4-aminobutanoic acid (485.32 mg, 4.71 mmol) in DMSO (7 mL) was added DIEA (2.34 g, 18.10 mmol, 3.15 mL). The mixture was stirred at 120° C. for 2 hours. LCMS showed the reaction was completed. To the mixture was added CH₃COOH to adjust pH<7. The resulting solution was purified by reverse column (TFA condition) (330 g Flash Column, Welch μltimate XB_C18 20-40 μm; 120 A, 40% 10 min;% min 60 min @100 mL/min) to afford (two batches of the product with different purity) 4-((2-(2,6-dioxopiperidin-3-yl)-1,3-dioxoisoindolin-4-yl)amino) butanoic acid (480 mg, 601.11 μmol, 16.60% yield, 45% purity) and 4-((2-(2,6-dioxopiperidin-3-yl)-1,3-dioxoisoindolin-4-yl)amino) butanoic acid (410 mg, 353.71 μmol, 9.77% yield, 31% purity) as a yellow solid. MS(M+H)⁺=360.1

### Step 2. Synthesis of tert-butyl 4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzamido)piperazine-1-carboxylate (6)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (150 mg, 322.29 μmol) in DMF (2 mL) were added HATU (183.81 mg, 483.43 μmol) and DIEA (166.61 mg, 1.29 mmol, 224.55 μL). The mixture was stirred at 25° C. for 15 minutes. To the mixture was added tert-butyl 4-aminopiperazine-1-carboxylate (77.84 mg, 386.74 μmol) and the resulting mixture was stirred at 25° C. for 12 hours. LCMS showed the reaction was completed. The mixture was diluted with H₂O (30 mL) and extracted with EtOAc (20 mL×3). The combined organic layers were washed with brine (10 mL×3), dried over Na₂SO₄, filtered and concentrated in vacuum. The residue was purified by flash silica gel chromatography (12 g SepaFlash® Silica Flash Column, Eluent of 25˜75% EtOAc/Petroleum ether gradient @80 mL/min) to afford tert-butyl 4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzamido)piperazine-1-carboxylate (160 mg, 246.66 μmol, 76.53% yield) as a brown solid. MS(M+H)⁺=649.2

### Step 3. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxy-N-(piperazin-1-yl)benzamide (7)

To a solution of tert-butyl 4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzamido)piperazine-1-carboxylate (160 mg, 246.66 μmol) in dioxane (3 mL) was added HCl/dioxane (4 M, 5 mL). The mixture was stirred at 25° C. for 2 hours. LCMS showed a main peak with desired mass. The reaction mixture was concentrated in vacuum to afford 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxy-N-(piperazin-1-yl)benzamide (180 mg, HCl salt) as a white solid. MS(M+H)⁺=549.2

### Step 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(4-((2-(2,6-dioxopiperidin-3-yl)-1,3-dioxoisoindolin-4-yl)amino)butanoyl)piperazin-1-yl)-2-fluoro-5-methoxybenzamide (Compound 29)

To a solution of 4-((2-(2,6-dioxopiperidin-3-yl)-1,3-dioxoisoindolin-4-yl)amino)butanoic acid (170 mg, 473.10 μmol) in DMF (2 mL) were added HATU (269.83 mg, 709.65 μmol) and DIEA (305.72 mg, 2.37 mmol, 412.03 μL). The mixture was stirred at 25° C. for 15 minutes. To the mixture was added 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxy-N-(piperazin-1-yl)benzamide (166.06 mg, 283.86 μmol, HCl salt). The resulting mixture was stirred at 25° C. for 1 hour. LCMS showed the reaction was completed. To the reaction mixture was added CH₃COOH to adjust pH<7. The resulting mixture was purified by prep-HPLC (column: Phenomenex luna C18 150*40 mm*15 μm; mobile phase: [water(FA)-ACN]; B %: 38%-68%, 10 min) followed by prep-TLC (SiO₂, Dichloromethane:Methanol=10:1) and prep-HPLC (column: Waters Xbridge 150*25 mm*5 μm; mobile phase: [water(NH₄HCO₃)-ACN]; B %: 38%-68%, 10 min). The eluent was freeze-dried to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(4-((2-(2,6-dioxopiperidin-3-yl)-1,3-dioxoisoindolin-4-yl)amino)butanoyl)piperazin-1-yl)-2-fluoro-5-methoxybenzamide (16.3 mg, 17.95 μmol, 3.79% yield, 98% purity) as a yellow solid. MS(M+H)⁺=890.2

¹H NMR (400 MHz, DMSO-d6) δ=11.12 (s, 1H), 9.23 (s, 1H), 8.41-8.19 (m, 2H), 8.12-7.95 (m, 1H), 7.60 (t, J=7.8 Hz, 1H), 7.28-7.09 (m, 2H), 7.02 (d, J=7.0 Hz, 1H), 6.68 (t, J=5.9 Hz, 1H), 5.05 (dd, J=5.3, 13.0 Hz, 1H), 4.90-4.72 (m, 1H), 4.08 (t, J=13.8 Hz, 2H), 3.98 (s, 3H), 3.70-3.43 (m, 4H), 2.98-2.73 (m, 5H), 2.63-2.50 (m, 7H), 2.41-2.35 (m, 2H), 2.07-1.88 (m, 3H), 1.86-1.76 (m, 2H), 1.74-1.68 (m, 2H), 1.52-1.38 (m, 4H).

### Example 30. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1,3-dioxoisoindolin-4-yl)oxy)butanoyl)piperidin-4-yl)-2-fluoro-5-methoxybenzamide (Compound 30)

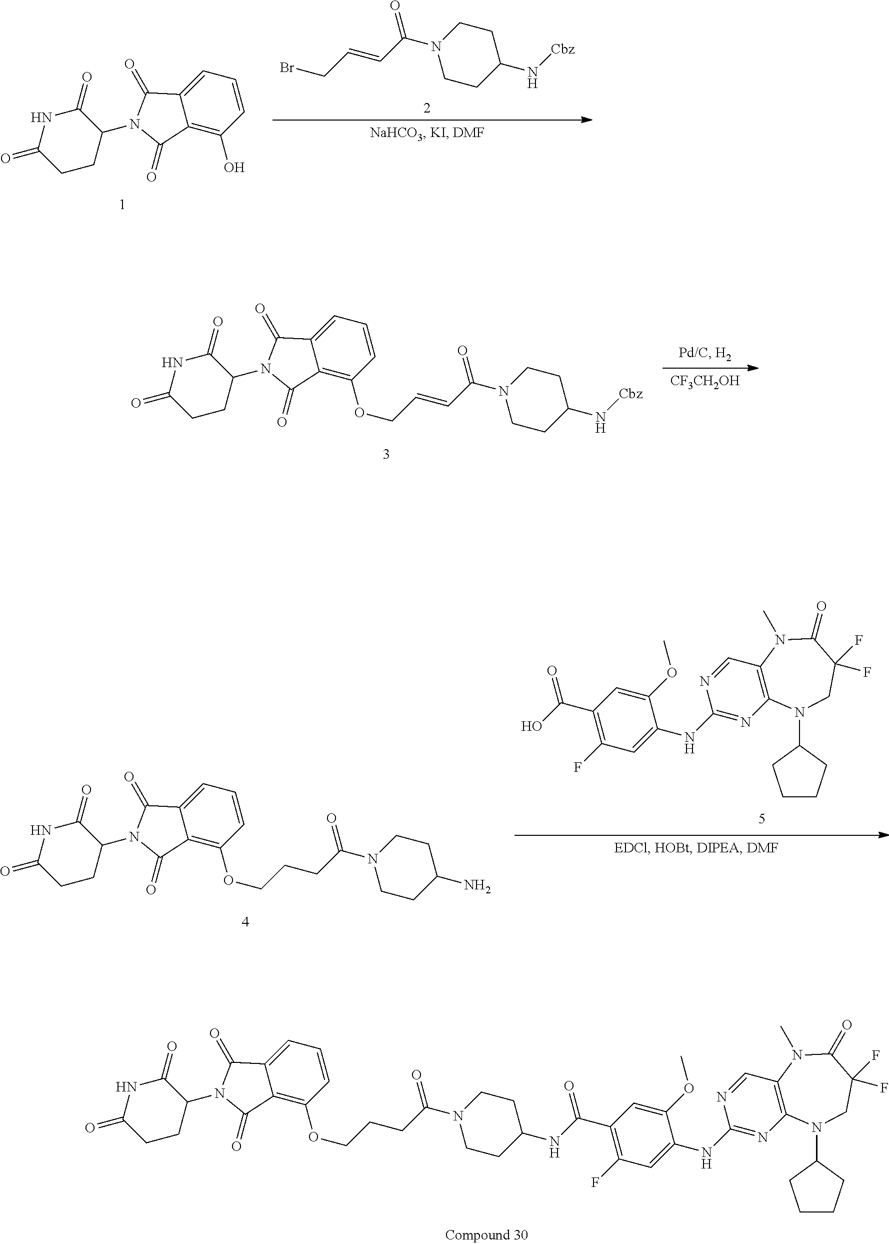

### Step 1. Synthesis of benzyl (E)-(1-(4-((2-(2,6-dioxopiperidin-3-yl)-1,3-dioxoisoindolin-4-yl)oxy)but-2-enoyl)piperidin-4-yl)carbamate (3)

To a solution of 2-(2,6-dioxopiperidin-3-yl)-4-hydroxyisoindoline-1,3-dione (200 mg, 729.32 μmol) and benzyl (E)-(1-(4-bromobut-2-enoyl)piperidin-4-yl)carbamate (278.06 mg, 729.32 μmol) in DMF (5 mL) were added NaHCO₃ (367.61 mg, 4.38 mmol, 170.19 μL) and KI (121.07 mg, 729.32 μmol). The mixture was stirred at 70° C. for 10 h. LCMS showed 2-(2,6-dioxopiperidin-3-yl)-4-hydroxyisoindoline-1,3-dione was consumed completely, a peak (47%) with desired mass. The reaction mixture was diluted with water (100 mL) and extracted with EtOAc (30 mL×3). The combined organic layers were washed with brine (30 mL), dried over anhydrous sodium sulfate, filtered and concentrated under reduced pressure. The residue was purified by column chromatography (SiO₂, Petroleum ether/EtOAc=4/1 to 3/1) to afford benzyl (E)-(1-(4-((2-(2,6-dioxopiperidin-3-yl)-1,3-dioxoisoindolin-4-yl)oxy)but-2-enoyl)piperidin-4-yl)carbamate (200 mg, 344.60 μmol, 47.25% yield, 99% purity) as a white solid. MS(M+H)⁺=575.1

### Step 2. Synthesis of 4-(4-(4-aminopiperidin-1-yl)-4-oxobutoxy)-2-(2,6-dioxopiperidin-3-yl)isoindoline-1,3-dione (4)

To a solution of benzyl (E)-(1-(4-((2-(2,6-dioxopiperidin-3-yl)-1,3-dioxoisoindolin-4-yl)oxy)but-2-enoyl)piperidin-4-yl)carbamate (290 mg, 504.72 μmol) in CF₃CH₂OH (6 mL) was added Pd/C (10%, 50 mg) under N₂ atmosphere. The suspension was degassed and purged with H₂ for 3 times. The mixture was stirred at 25° C. for 10 h under H₂ (15 Psi). LCMS showed benzyl (E)-(1-(4-((2-(2,6-dioxopiperidin-3-yl)-1,3-dioxoisoindolin-4-yl)oxy)but-2-enoyl)piperidin-4-yl)carbamate was consumed completely and a peak (88%) with desired mass. The reaction mixture was filtered and filter cake was washed with EtOAc (100 mL), the filtrate was concentrated in vacuo to afford 4-(4-(4-aminopiperidin-1-yl)-4-oxobutoxy)-2-(2,6-dioxopiperidin-3-yl)isoindoline-1,3-dione (210 mg, crude) as a green solid. MS(M+H)⁺=443.3

### Step 3. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1,3-dioxoisoindolin-4-yl)oxy)butanoyl)piperidin-4-yl)-2-fluoro-5-methoxybenzamide (Compound 30)

To a solution of 4-(4-(4-aminopiperidin-1-yl)-4-oxobutoxy)-2-(2,6-dioxopiperidin-3-yl)isoindoline-1,3-dione (100 mg, crude) and 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (105.19 mg, 226.01 μmol) in DMF (3 mL) were added DIPEA (87.63 mg, 678.02 μmol, 118.10 μL) and EDCI (64.99 mg, 339.01 μmol), HOBt (45.81 mg, 339.01 μmol). The mixture was stirred at 25° C. for 2 h. LCMS showed 4-(4-(4-aminopiperidin-1-yl)-4-oxobutoxy)-2-(2,6-dioxopiperidin-3-yl)isoindoline-1,3-dione was consumed completely and a peak (38%) with desired mass. The reaction mixture was diluted with water (50 mL) and extracted with EtOAc (20 mL×3). The combined organic layers were washed with brine (20 mL), dried over anhydrous sodium sulfate, filtered and concentrated under reduced pressure. The residue was purified by column chromatography (SiO₂, MeOH/EtOAc=1/10 to 1/5) and prep-HPLC (neutral condition: column: Waters Xbridge 150×25 mm×5 μm; mobile phase: [water(NH₄HCO₃)-ACN]; B %: 42%-72%, 8 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1,3-dioxoisoindolin-4-yl)oxy)butanoyl)piperidin-4-yl)-2-fluoro-5-methoxybenzamide (12.5 mg, 13.63 μmol, 6.03% yield, 96.8% purity) as a white solid. MS(M+H)⁺=890.6.

¹H NMR (400 MHz, DMSO-d₆) δ=11.25-10.98 (m, 1H), 8.34-8.21 (m, 2H), 8.05 (s, 1H), 7.96 (dd, J=3.2, 7.6 Hz, 1H), 7.83 (dd, J=7.5, 8.3 Hz, 1H), 7.55 (d, J=8.6 Hz, 1H), 7.46 (d, J=7.2 Hz, 1H), 7.21 (d, J=6.7 Hz, 1H), 5.09 (dd, J=5.3, 12.9 Hz, 1H), 4.91-4.73 (m, 1H), 4.42-4.20 (m, 3H), 4.17-4.00 (m, 3H), 3.96-3.85 (m, 4H), 3.34 (s, 3H), 3.20-3.13 (m, 1H), 2.96-2.84 (m, 1H), 2.81-2.72 (m, 1H), 2.63-2.56 (m, 4H), 2.07-1.93 (m, 5H), 1.91-1.78 (m, 2H), 1.76-1.57 (m, 6H), 1.53-1.34 (m, 2H).

### Example 31. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1,3-dioxoisoindolin-4-yl)oxy)butanoyl)piperidin-4-yl)-3-methoxybenzamide (Compound 31)

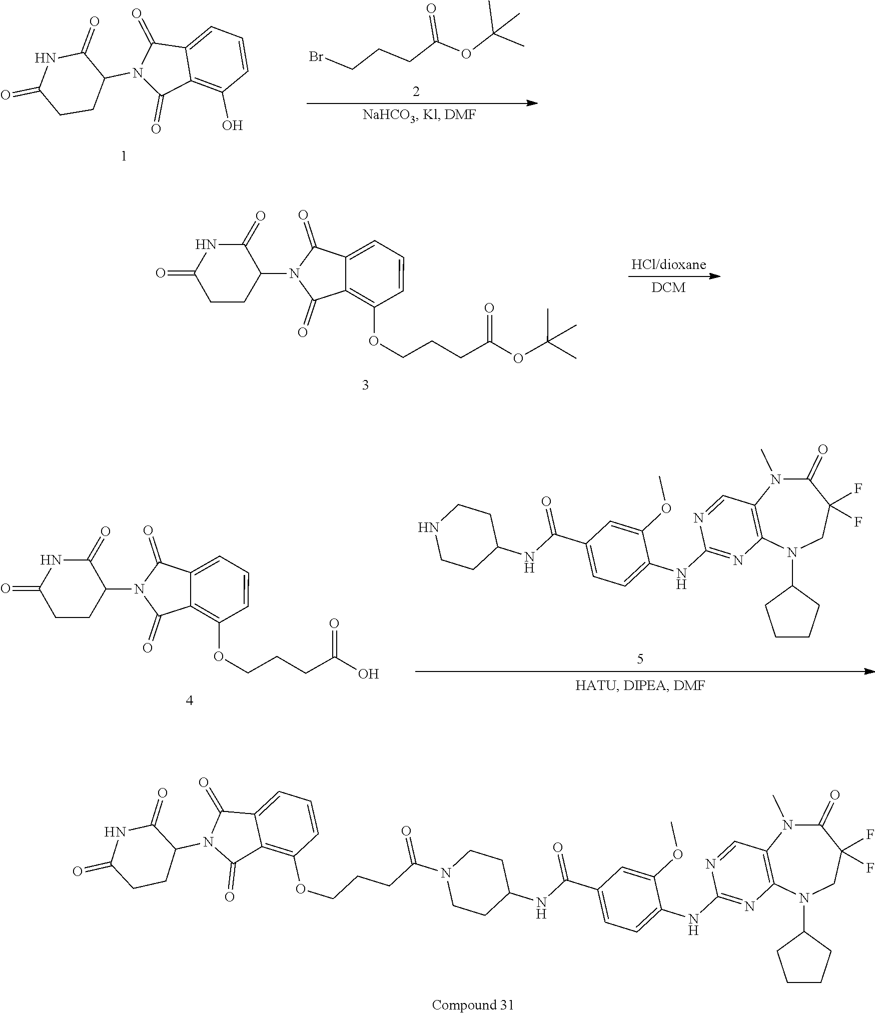 [SMILES: O=C1CCC(N2C(=O)c3cccc(O)c3C2=O)C(=O)N1 ; CC(C)(C)OC(=O)CCCBr ; CC(C)(C)OC(=O)CCCOc1cccc2c1C(=O)N(C1CCC(=O)NC1=O)C2=O ; O=C(O)CCCOc1cccc2c1C(=O)N(C1CCC(=O)NC1=O)C2=O ; COc1cc(C(=O)NC2CCNCC2)ccc1Nc1ncc2c(n1)N(C1CCCC1)CC(F)(F)C(=O)N2C ; COc1cc(C(=O)NC2CCN(C(=O)CCCOc3cccc4c3C(=O)N(C3CCC(=O)NC3=O)C4=O)CC2)ccc1Nc1ncc2c(n1)N(C1CCCC1)CC(F)(F)C(=O)N2C]

The compound 31 was synthesized by the method described in the scheme similar to the method described in Example 26.

MS(M+H)⁺=872.0, ¹H NMR (400 MHz, DMSO-d₆) δ=11.16-10.96 (m, 1H), 8.32-8.24 (m, 2H), 8.14 (d, J=7.8 Hz, 1H), 7.97 (s, 1H), 7.87-7.78 (m, 1H), 7.59-7.41 (m, 4H), 5.09 (dd, J=5.4, 12.9 Hz, 1H), 4.81-4.68 (m, 1H), 4.48-4.39 (m, 1H), 4.27 (t, J=6.3 Hz, 2H), 4.12-4.00 (m, 3H), 3.97-3.92 (m, 4H), 3.32 (s, 3H), 3.20-3.07 (m, 1H), 2.94-2.82 (m, 1H), 2.75-2.65 (m, 1H), 2.62-2.54 (m, 4H), 2.06-1.80 (m, 7H), 1.75-1.67 (m, 2H), 1.66-1.55 (m, 4H), 1.52-1.36 (m, 2H).

### Example 32. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1-oxoisoindolin-4-yl)oxy)butanoyl)piperidin-4-yl)-3-methoxybenzamide (Compound 32)

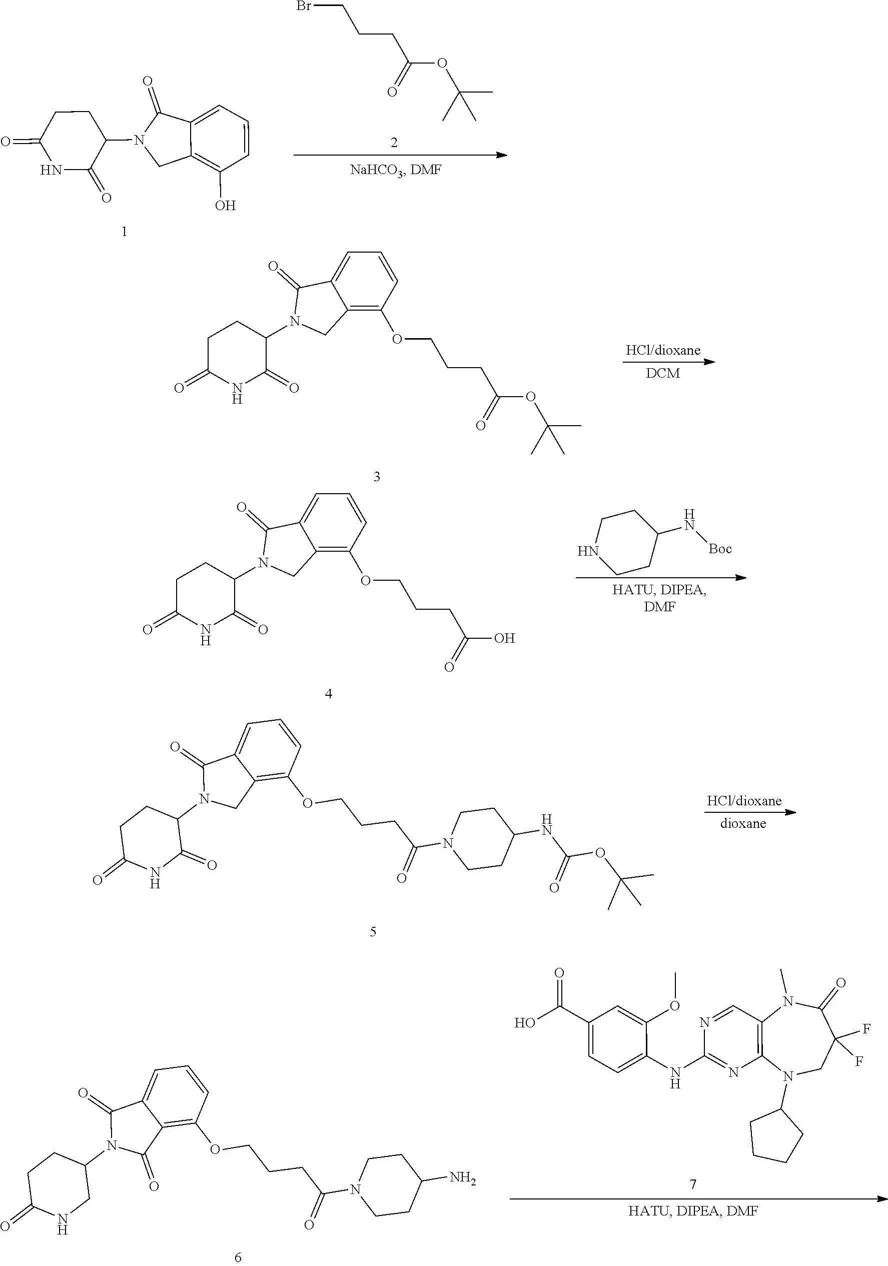

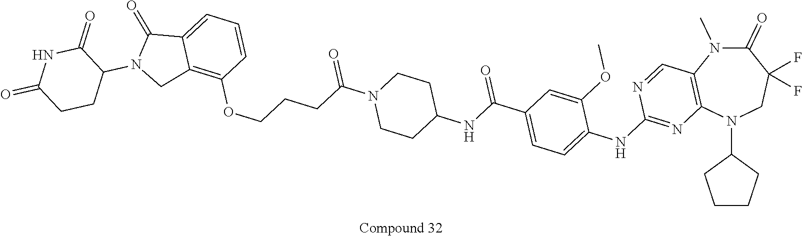 [SMILES: COc1cc(C(=O)NC2CCN(C(=O)CCCOc3cccc4c3CN(C3CCC(=O)NC3=O)C4=O)CC2)ccc1Nc1ncc2c(n1)N(C1CCCC1)CC(F)(F)C(=O)N2C]

The compound 32 was synthesized by the method described in the scheme similar to the method described in Example 26.

MS (M+H)⁺=858.4, ¹H NMR (400 MHz, DMSO-d₆) δ=10.98 (s, 1H), 8.31-8.22 (m, 2H), 8.14 (d, J=7.6 Hz, 1H), 7.99 (s, 1H), 7.53-7.43 (m, 3H), 7.31 (d, J=7.5 Hz, 1H), 7.25 (d, J=8.1 Hz, 1H), 5.11 (dd, J=5.1, 13.3 Hz, 1H), 4.77 (t, J=8.2 Hz, 1H), 4.40-4.37 (m, 2H), 4.27-4.21 (m, 1H), 4.16 (t, J=6.4 Hz, 2H), 4.05 (t, J=14.1 Hz, 2H), 3.93 (s, 3H), 3.32-3.31 (m, 3H), 3.12 (t, J=11.9 Hz, 1H), 2.98-2.84 (m, 1H), 2.73-2.66 (m, 1H), 2.61-2.54 (m, 1H), 2.44-2.42 (m, 4H), 2.03-1.94 (m, 6H), 1.88-1.82 (m, 2H), 1.75-1.68 (m, 2H), 1.65-1.42 (m, 6H).

### Example 33. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1,3-dioxoisoindolin-4-yl)amino)butanoyl)piperidin-4-yl)-2-fluoro-5-methoxybenzamide (Compound 33)

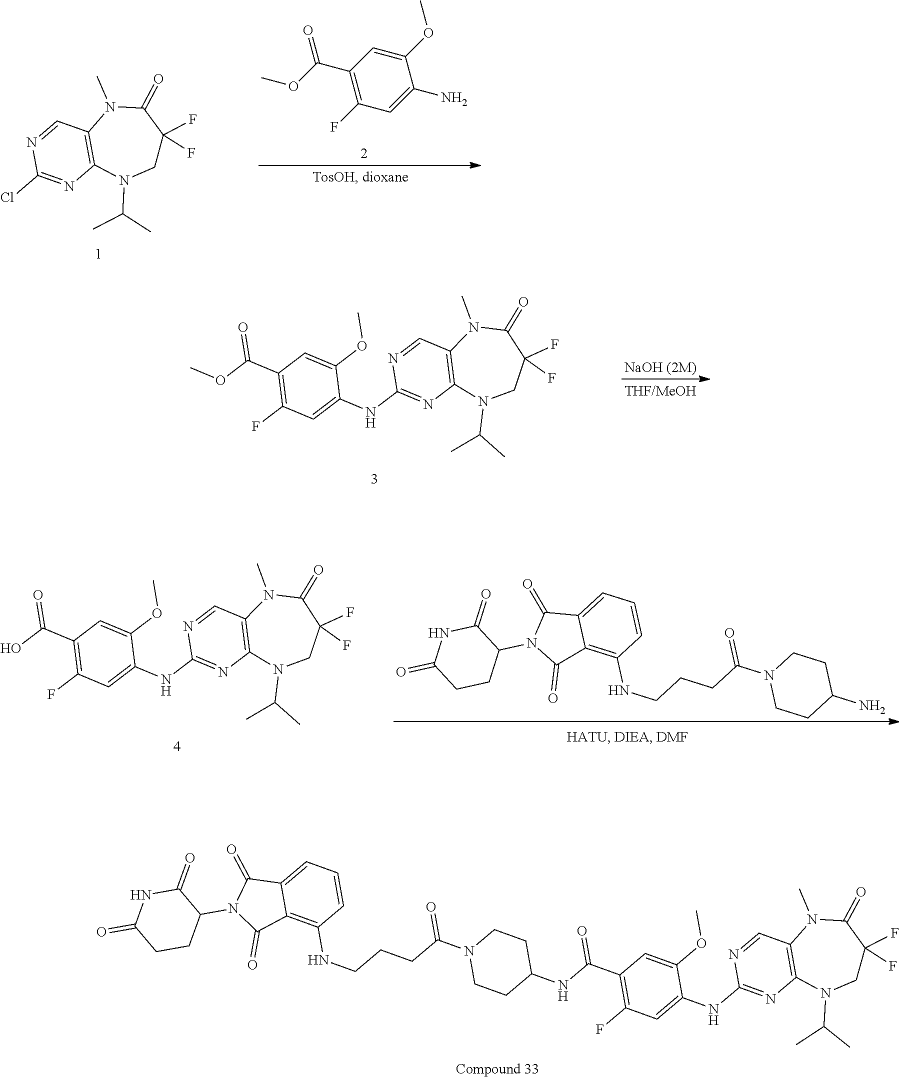 [SMILES: CC(C)N1CC(F)(F)C(=O)N(C)c2cnc(Cl)nc21 ; COC(=O)c1cc(OC)c(N)cc1F ; COC(=O)c1cc(OC)c(Nc2ncc3c(n2)N(C(C)C)CC(F)(F)C(=O)N3C)cc1F ; COc1cc(C(=O)O)c(F)cc1Nc1ncc2c(n1)N(C(C)C)CC(F)(F)C(=O)N2C ; NC1CCN(C(=O)CCCNc2cccc3c2C(=O)N(C2CCC(=O)NC2=O)C3=O)CC1 ; COc1cc(C(=O)NC2CCN(C(=O)CCCNc3cccc4c3C(=O)N(C3CCC(=O)NC3=O)C4=O)CC2)c(F)cc1Nc1ncc2c(n1)N(C(C)C)CC(F)(F)C(=O)N2C]

### Step 1. Synthesis of methyl 4-((7,7-difluoro-9-isopropyl-5-methyl-6-oxo-6,7,8,9-tetrahydro-5H-pyrimido[4,5-b][1,4]diazepin-2-yl)amino)-2-fluoro-5-methoxybenzoate (3)

A mixture of 2-chloro-7,7-difluoro-9-isopropyl-5-methyl-8,9-dihydro-5H-pyrimido[4,5-b][1,4]diazepin-6(7H)-one (1 g, 3.44 mmol), methyl 4-amino-2-fluoro-5-methoxybenzoate (1.03 g, 5.16 mmol) and TosOH (1.78 g, 10.32 mmol) in dioxane (15 mL) was stirred at 100° C. for 16 hours. LCMS showed trace of the 2-chloro-7,7-difluoro-9-isopropyl-5-methyl-8,9-dihydro-5H-pyrimido[4,5-b][1,4]diazepin-6(7H)-one remained and 63% of desired mass was detected. The mixture was poured in to the H₂O (100 mL), the mixture was extracted with EtOAc (100 mL×3). The combined organic layers were washed with saturated Na₂CO₃ solution (100 mL×2), dried over Na₂SO₄, filtered and concentrated in vacuo. The residue was purified by flash silica gel chromatography (40 g SepaFlash® Silica Flash Column, Eluent of 10˜80% EtOAc/Petroleum ether gradient @100 mL/min) to afford the crude product, the crude product was triturated with MTBE (15 mL) for 5 minutes, the suspension was filtered and the filter cake was washed with MTBE (15 mL), the filter cake was collected and dried to afford methyl 4-((7,7-difluoro-9-isopropyl-5-methyl-6-oxo-6,7,8,9-tetrahydro-5H-pyrimido[4,5-b][1,4]diazepin-2-yl)amino)-2-fluoro-5-methoxybenzoate (980 mg, 2.16 mmol, 62.83% yield) as a white solid. MS(M+H)⁺=454.1

¹H NMR (400 MHz, DMSO-d₆) δ=8.35 (d, J=13.9 Hz, 1H), 8.28 (s, 1H), 8.09 (s, 1H), 7.36 (d, J=6.7 Hz, 1H), 4.98-4.83 (m, 1H), 4.15-4.01 (m, 2H), 3.93 (s, 3H), 3.83 (s, 3H), 3.23 (s, 3H), 1.25 (d, J=6.7 Hz, 6H).

### Step 2. Synthesis of 4-((7,7-difluoro-9-isopropyl-5-methyl-6-oxo-6,7,8,9-tetrahydro-5H-pyrimido[4,5-b][1,4]diazepin-2-yl)amino)-2-fluoro-5-methoxybenzoic acid (4)

To a mixture of methyl 4-((7,7-difluoro-9-isopropyl-5-methyl-6-oxo-6,7,8,9-tetrahydro-5H-pyrimido[4,5-b][1, 4]diazepin-2-yl)amino)-2-fluoro-5-methoxybenzoate (980 mg, 2.16 mmol) in THF (4 mL) and MeOH (4 mL) was added NaOH (2 M in H₂O, 4 mL), the mixture was stirred at 25° C. for 12 hours. LCMS showed the starting material was consumed completely and 98% of desired mass was detected. The mixture was concentrated in vacuum to remove most of the organic solvent. The residue was poured into H₂O (30 mL), to the mixture was added HCl (12 M) to adjust pH<3, the suspension was filtered and the filter cake was washed with H₂O (30 mL), the filter cake was collected and dried. The residue was diluted with HCl solution (20 mL, 4 M) and stirred at 25° C. for 1 hour, the suspension was concentrated in vacuum at 70° C. to afford 4-((7,7-difluoro-9-isopropyl-5-methyl-6-oxo-6,7,8,9-tetrahydro-5H-pyrimido[4,5-b][1, 4]diazepin-2-yl)amino)-2-fluoro-5-methoxybenzoic acid (1 g, crude) as an off-white solid, which was used directly. MS(M+H)⁺=440.0

¹H NMR (400 MHz, DMSO-d₆) δ=9.37 (br s, 1H), 8.41 (s, 1H), 8.10 (br d, J=13.1 Hz, 1H), 7.41 (br d, J=6.6 Hz, 1H), 4.98-4.91 (m, 1H), 4.24 (t, J=12.4 Hz, 2H), 3.91 (s, 3H), 3.33 (s, 3H), 1.28 (br d, J=6.6 Hz, 6H).

### Step 3. Synthesis of 4-((7,7-difluoro-9-isopropyl-5-methyl-6-oxo-6,7,8,9-tetrahydro-5H-pyrimido[4,5-b][1, 4]diazepin-2-yl)amino)-N-(1-(4-((2-(2,6-dioxopiperidin-3-yl)-1,3-dioxoisoindolin-4-yl)amino)butanoyl)piperidin-4-yl)-2-fluoro-5-methoxybenzamide (Compound 33)

To a solution of 4-((7,7-difluoro-9-isopropyl-5-methyl-6-oxo-6,7,8,9-tetrahydro-5H-pyrimido[4,5-b][1, 4]diazepin-2-yl)amino)-2-fluoro-5-methoxybenzoic acid (200 mg, 455.18 μmol) in DMF (2 mL) were added DIEA (235.31 mg, 1.82 mmol, 317.14 μL) and HATU (224.99 mg, 591.73 μmol), the mixture was stirred at 25° C. for 15 minutes, to the mixture was added 4-((4-(4-aminopiperidin-1-yl)-4-oxobutyl)amino)-2-(2,6-dioxopiperidin-3-yl)isoindoline-1,3-dione (217.55 mg, 455.18 μmol, HCl), the resulting mixture was stirred at 25° C. for 1 hour. LCMS showed 4-((7,7-difluoro-9-isopropyl-5-methyl-6-oxo-6,7,8,9-tetrahydro-5H-pyrimido[4,5-b][1, 4]diazepin-2-yl)amino)-2-fluoro-5-methoxybenzoic acid was consumed completely and 90% of desired mass was detected. The reaction mixture was combined with another batch (50 mg) for further work-up and purification. To the mixture was added CH₃COOH to adjust pH<7. The resulting mixture was purified by prep-HPLC (column: Waters Xbridge 150*25 mm*5 μm; mobile phase: [water(NH₄HCO₃)-ACN]; B %: 40%-70%, 9 min) followed by prep-HPLC (column: Phenomenex luna C₁₈ 150*40 mm*15 μm; mobile phase: [water(FA)-ACN]; B %: 38%-68%, 10 min), the eluent was freeze-dried to afford 4-((7,7-difluoro-9-isopropyl-5-methyl-6-oxo-6,7,8,9-tetrahydro-5H-pyrimido[4,5-b][1, 4]diazepin-2-yl)amino)-N-(1-(4-((2-(2,6-dioxopiperidin-3-yl)-1,3-dioxoisoindolin-4-yl)amino)butanoyl)piperidin-4-yl)-2-fluoro-5-methoxybenzamide (196.1 mg, 222.72 μmol, 48.93% yield, 98% purity) as a yellow solid. MS(M+H)⁺=863.1

¹H NMR (400 MHz, DMSO-d₆) δ=11.24-10.90 (m, 1H), 8.38-8.22 (m, 2H), 8.08-7.89 (m, 2H), 7.68-7.55 (m, 1H), 7.29-7.14 (m, 2H), 7.02 (d, J=7.0 Hz, 1H), 6.69 (t, J=6.0 Hz, 1H), 5.05 (dd, J=5.3, 12.9 Hz, 1H), 4.95-4.80 (m, 1H), 4.32 (d, J=13.4 Hz, 1H), 4.13-3.97 (m, 3H), 3.91 (s, 3H), 3.89-3.77 (m, 1H), 3.33-3.31 (m, 5H), 3.13 (t, J=11.8 Hz, 1H), 2.96-2.82 (m, 1H), 2.75 (t, J=11.6 Hz, 1H), 2.63-2.53 (m, 2H), 2.42 (t, J=7.0 Hz, 2H), 2.07-1.97 (m, 1H), 1.91-1.72 (m, 4H), 1.49-1.33 (m, 2H), 1.25 (d, J=6.7 Hz, 6H).

### Example 34. Synthesis of 4-((9-cyclopropyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1,3-dioxoisoindolin-4-yl)amino)butanoyl)piperidin-4-yl)-2-fluoro-5-methoxybenzamide (Compound 34)

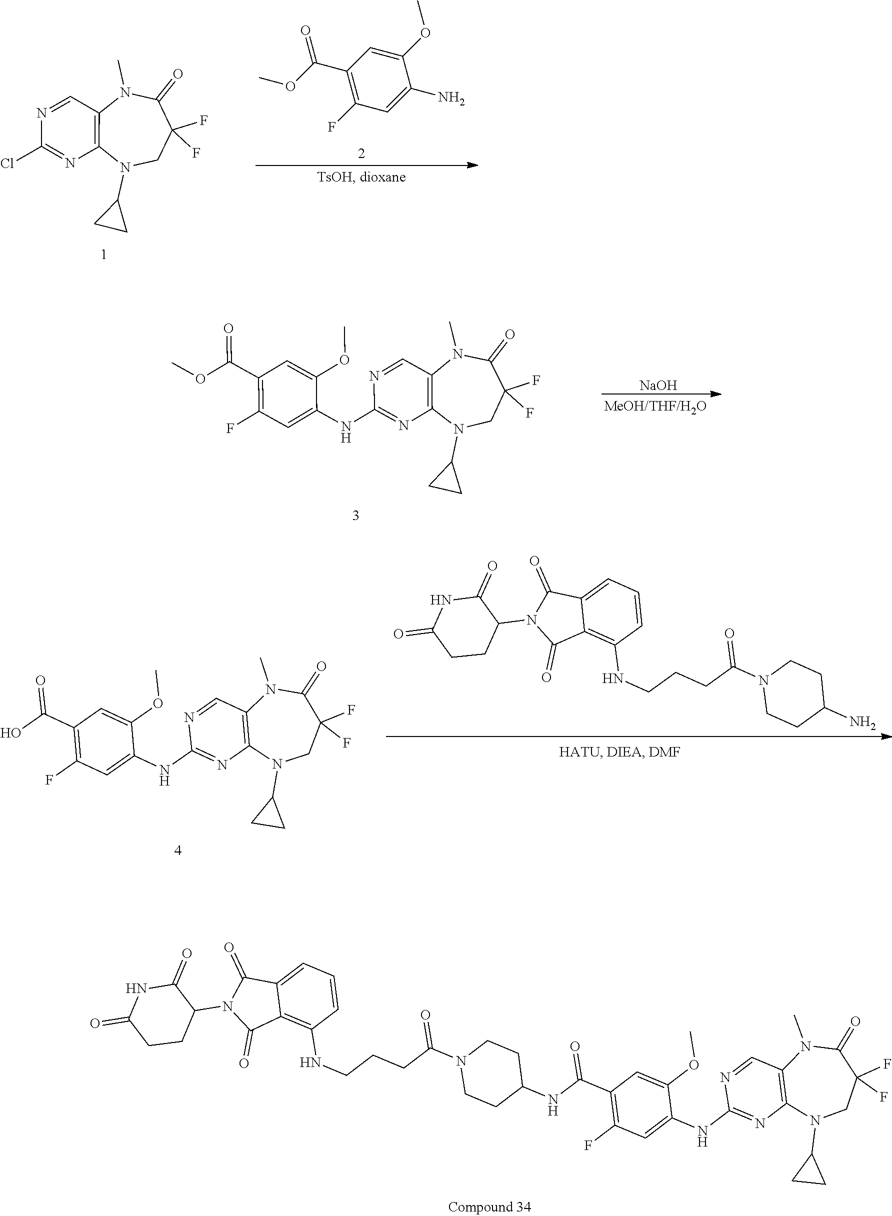

The compound 34 was synthesized by the method described in the scheme similar to the method described in Example 33.

MS(M+H)⁺=861.7, ¹H NMR (400 MHz, DMSO-d₆) δ=11.33-10.81 (m, 1H), 8.55 (d, J=13.7 Hz, 1H), 8.38 (s, 1H), 8.13 (s, 1H), 7.93 (dd, J=3.5, 7.5 Hz, 1H), 7.59 (dd, J=7.3, 8.4 Hz, 1H), 7.29-7.12 (m, 2H), 7.02 (d, J=7.0 Hz, 1H), 6.67 (t, J=6.0 Hz, 1H), 5.05 (dd, J=5.4, 12.8 Hz, 1H), 4.36-4.17 (m, 3H), 4.07-3.97 (m, 1H), 3.92 (s, 3H), 3.84 (d, J=14.2 Hz, 1H), 3.32-3.28 (m, 5H), 3.19-3.05 (m, 1H), 2.96-2.82 (m, 2H), 2.75 (t, J=11.4 Hz, 1H), 2.64-2.53 (m, 2H), 2.41 (t, J=7.0 Hz, 2H), 2.07-1.96 (m, 1H), 1.92-1.69 (m, 4H), 1.53-1.29 (m, 2H), 0.92-0.78 (m, 2H), 0.77-0.63 (m, 2H).

### Example 35. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1,3-dioxoisoindolin-5-yl)amino)butanoyl)piperidin-4-yl)-2-fluoro-5-methoxybenzamide (Compound 35)

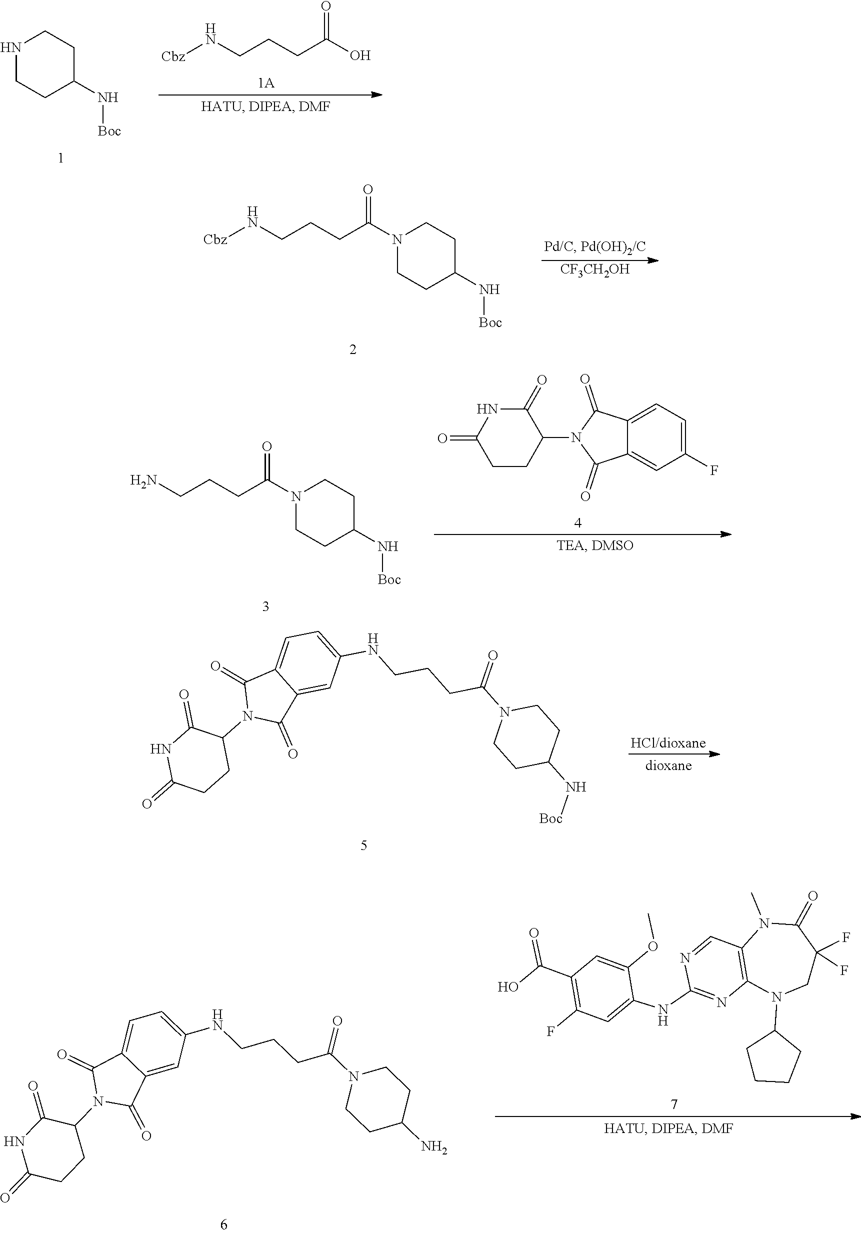

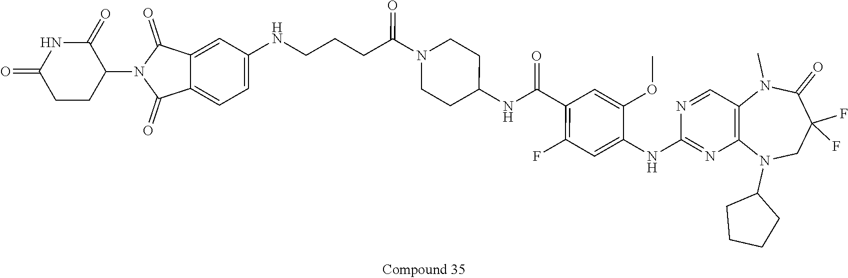 [SMILES: COc1cc(C(=O)NC2CCN(C(=O)CCCNc3ccc4c(c3)C(=O)N(C3CCC(=O)NC3=O)C4=O)CC2)c(F)cc1Nc1ncc2c(n1)N(C1CCCC1)CC(F)(F)C(=O)N2C]

The compound 35 was synthesized by the method described in the scheme similar to the method described in Example 24.

MS (M+H)⁺=889.6, ¹H NMR (400 MHz, DMSO-d₆) δ=11.06 (s, 1H), 8.33-8.22 (m, 2H), 8.05 (s, 1H), 8.00-7.94 (m, 1H), 7.58 (d, J=8.4 Hz, 1H), 7.24-7.16 (m, 2H), 6.97 (d, J=1.6 Hz, 1H), 6.90-6.83 (m, 1H), 5.10-4.97 (m, 1H), 4.89-4.78 (m, 1H), 4.40-4.28 (m, 1H), 4.15-3.99 (m, 3H), 3.92 (s, 3H), 3.90-3.81 (m, 1H), 3.47-3.40 (m, 1H), 3.35-3.34 (m, 3H), 3.26-3.11 (m, 2H), 2.98-2.69 (m, 4H), 2.47-2.44 (m, 2H), 2.02-1.94 (m, 3H), 1.89-1.79 (m, 4H), 1.74-1.58 (m, 6H), 1.52-1.32 (m, 2H).

### Example 36. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1-oxoisoindolin-5-yl)amino)butanoyl)piperidin-4-yl)-2-fluoro-5-methoxybenzamide (Compound 36)

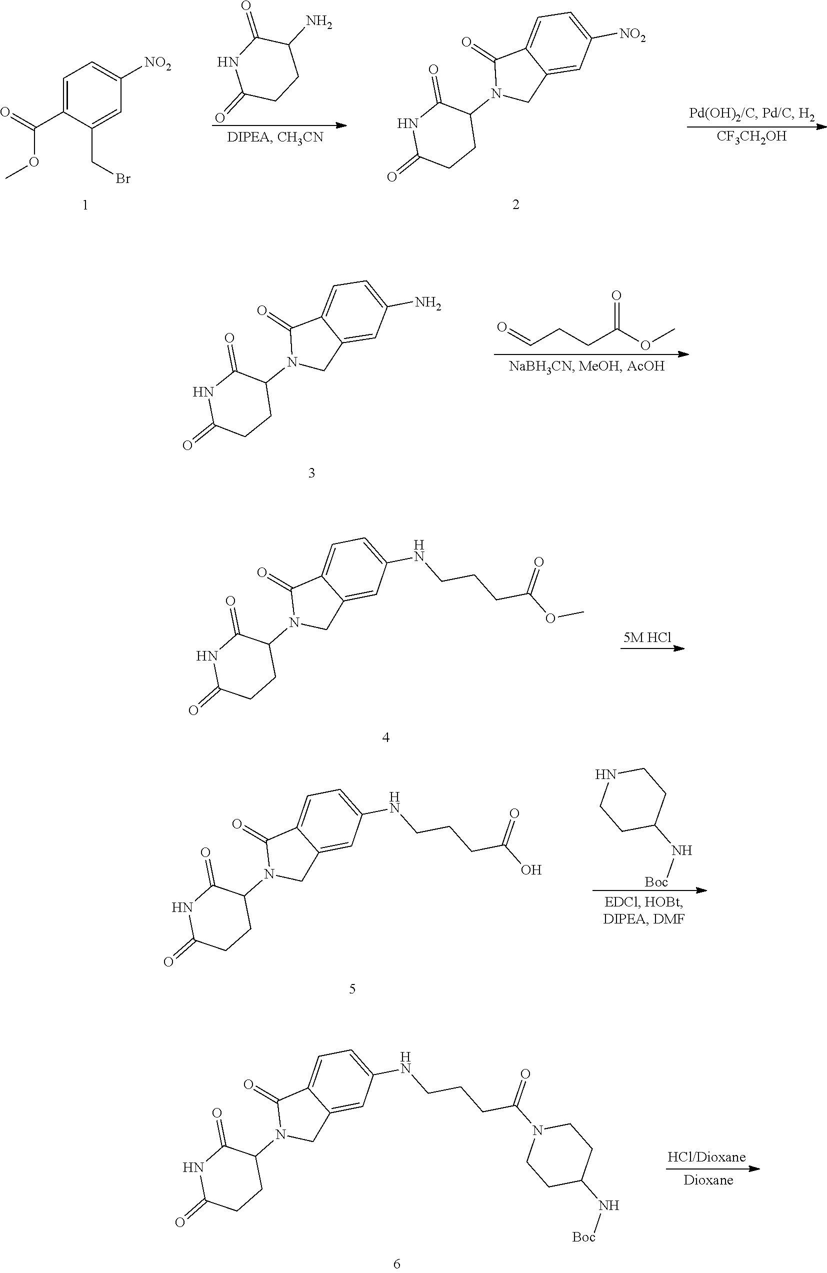

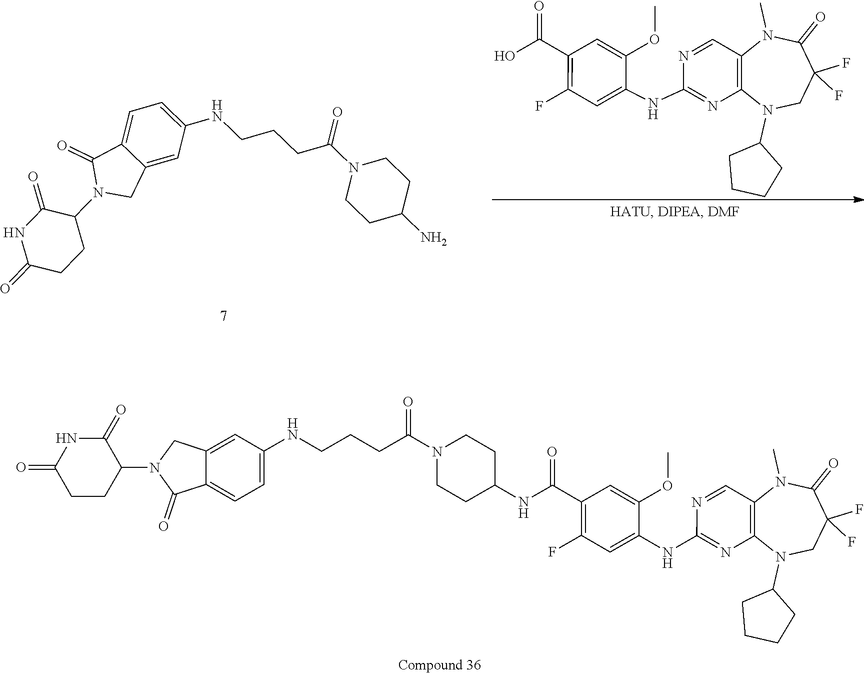 [SMILES: NC1CCN(C(=O)CCCNc2ccc3c(c2)CN(C2CCC(=O)NC2=O)C3=O)CC1 ; COc1cc(C(=O)O)c(F)cc1Nc1ncc2c(n1)N(C1CCCC1)CC(F)(F)C(=O)N2C ; COc1cc(C(=O)NC2CCN(C(=O)CCCNc3ccc4c(c3)CN(C3CCC(=O)NC3=O)C4=O)CC2)c(F)cc1Nc1ncc2c(n1)N(C1CCCC1)CC(F)(F)C(=O)N2C]

### Step 1. Synthesis of 3-(5-nitro-1-oxoisoindolin-2-yl)piperidine-2,6-dione (2)

To a solution of methyl methyl 2-(bromomethyl)-4-nitrobenzoate (2.8 g, 10.22 mmol) in CH₃CN (30 mL) was added DIPEA (5.94 g, 45.93 mmol, 8 mL) and 3-aminopiperidine-2,6-dione; hydrochloride (1.6 g, 9.72 mmol) at 25° C. The mixture was stirred at 90° C. for 16 hr under N₂ atmosphere. LCMS showed 34% peak with the desired mass. To the mixture was added H₂O (20 mL) and the mixture was stirred at 25° C. for 1 hr, The resulting mixture was filtered and the filter cake was washed with MTBE (30 mL×2), dried under reduced pressure to afford 3-(5-nitro-1-oxoisoindolin-2-yl)piperidine-2,6-dione (1.7 g, crude) as a gray solid. MS(M+H)⁺=290.1

¹H NMR (400 MHz, DMSO-d₆) δ=11.06 (s, 1H), 8.53 (s, 1H), 8.40-8.32 (m, 1H), 7.98 (d, J=8.4 Hz, 1H), 5.26-5.10 (m, 1H), 4.67-4.43 (m, 2H), 2.99-2.87 (m, 1H), 2.68-2.57 (m, 1H), 2.48-2.38 (m, 1H), 2.12-1.97 (m, 1H).

### Step 2. Synthesis of 3-(5-amino-1-oxoisoindolin-2-yl)piperidine-2,6-dione (3)

To a solution of 3-(5-nitro-1-oxoisoindolin-2-yl)piperidine-2,6-dione (1 g, 3.46 mmol) in CF₃CH₂OH (60 mL) was added Pd(OH)₂/C (200 mg, 20% purity) and Pd/C (200 mg, 10% purity), the mixture was stirred at 20° C. for 12 hr under H₂ atmosphere (15 Psi). LCMS showed main peak with the desired mass. The reaction mixture was filtered and the filtrate was concentrated under reduced pressure to afford 3-(5-amino-1-oxoisoindolin-2-yl)piperidine-2,6-dione (500 mg, crude) was obtained as a yellow solid. MS(M+H)⁺=260.2

¹H NMR (400 MHz, DMSO-d₆) δ=10.93 (s, 1H), 7.86-7.72 (m, 1H), 7.43-7.37 (m, 2H), 6.74-6.52 (m, 2H), 5.07-4.90 (m, 1H), 4.30-4.08 (m, 2H), 2.96-2.82 (m, 1H), 2.64-2.53 (m, 1H), 2.38-2.26 (m, 1H), 1.98-1.89 (m, 1H).

### Step 3. Synthesis of methyl 4-((2-(2,6-dioxopiperidin-3-yl)-1-oxoisoindolin-5-yl)amino)butanoate (4)

To a solution of 3-(5-amino-1-oxoisoindolin-2-yl)piperidine-2,6-dione (350 mg, 1.35 mmol) in MeOH (50 mL) were added methyl 4-oxobutanoate (160 mg, 1.38 mmol) and AcOH (81.07 mg, 1.35 mmol, 77.21 μL). The mixture was stirred at 25° C. for 2 hr. Then NaBH₃CN (900 mg, 14.32 mmol) was added to the mixture at 25° C., the mixture was stirred at 25° C. for 14 hr. LCMS showed main peak with the desired mass. To the reaction mixture was added H₂O (10 mL) and it was concentrated. The mixture was diluted with EtOAc (20 mL) and then NaHCO₃ (sat., 20 mL) was added to adjust the pH=9, The mixture was extracted with EtOAc (30 mL×3), then the combined organic layers were washed with brine 8 (40 mL×2), dried over Na₂SO₄, filtered and the filtrate was concentrated under reduced pressure. The residue was purified by flash silica gel chromatography (Biotage; 10 g SepaFlash® Silica Flash Column, Eluent of 30˜100% EtOAc:Petroleum ether gradient, 50 mL/min) to afford methyl 4-((2-(2,6-dioxopiperidin-3-yl)-1-oxoisoindolin-5-yl)amino)butanoate (480 mg, 1.34 mmol, 98.94% yield) as a light yellow oil. MS(M+H)⁺=360.2

### Step 4. Synthesis of 4-((2-(2,6-dioxopiperidin-3-yl)-1-oxoisoindolin-5-yl)amino)butanoic acid (5)

A mixture of methyl 4-((2-(2,6-dioxopiperidin-3-yl)-1-oxoisoindolin-5-yl)amino)butanoate (300 mg, 834.78 μmol) in HCl (5 M, 14.59 mL) was stirred at 25° C. for 2 hr under N₂ atmosphere. LCMS showed main peak with the desired mass. The reaction mixture was concentrated under reduced pressure followed by lyophilization to afford 4-((2-(2,6-dioxopiperidin-3-yl)-1-oxoisoindolin-5-yl)amino)butanoic acid (280 mg, crude) as a light yellow oil. MS(M+H)⁺=346.1

### Step 5. Synthesis of tert-butyl (1-(4-((2-(2,6-dioxopiperidin-3-yl)-1-oxoisoindolin-5-yl)amino)butanoyl)piperidin-4-yl)carbamate (6)

To a solution of 4-((2-(2,6-dioxopiperidin-3-yl)-1-oxoisoindolin-5-yl)amino)butanoic acid (240 mg, 694.95 μmol) in DMF (15 mL) was added DIPEA (534.24 mg, 4.13 mmol, 720.00 μL), HOBt (180.00 mg, 1.33 mmol), EDCI (240.00 mg, 1.25 mmol) and tert-butyl piperidin-4-ylcarbamate (216.00 mg, 1.08 mmol) at 25° C. The mixture was stirred at 25° C. for 16 h under N₂ atmosphere. LCMS showed 68% peak with the desired mass and no peak with the starting material. To the reaction mixture was added H₂O (20 mL), the mixture was extracted with EtOAc (40 mL×2), the combined organic layers were washed with brine (30 mL×3), dried over Na₂SO₄, filtered and the filtrate was concentrated under reduced pressure. The residue was purified by flash silica gel chromatography (Biotage; 10 g SepaFlash® Silica Flash Column, Eluent of 0˜20% Methanol: EtOAc gradient, 50 mL/min) to afford tert-butyl (1-(4-((2-(2,6-dioxopiperidin-3-yl)-1-oxoisoindolin-5-yl)amino)butanoyl)piperidin-4-yl)carbamate (100 mg, 189.53 μmol, 27.27% yield) as a yellow solid. MS(M+H)⁺=528.3

¹H NMR (400 MHz, DMSO-d₆) δ=10.92 (s, 1H), 7.39 (d, J=8.3 Hz, 1H), 6.93-6.80 (m, 1H), 6.70-6.56 (m, 2H), 6.41 (t, J=5.4 Hz, 1H), 5.06-4.96 (m, 1H), 4.30-4.20 (m, 2H), 4.19-4.10 (m, 1H), 3.85-3.75 (m, 1H), 3.53-3.43 (m, 1H), 3.11-3.05 (m, 2H), 2.93-2.85 (m, 1H), 2.73-2.59 (m, 2H), 2.43-2.39 (m, 2H), 1.98-1.91 (m, 1H), 1.80-1.68 (m, 4H), 1.39 (s, 9H), 1.38-1.32 (m, 2H), 1.29-1.18 (m, 2H).

### Step 6. Synthesis of 3-(5-((4-(4-aminopiperidin-1-yl)-4-oxobutyl)amino)-1-oxoisoindolin-2-yl)piperidine-2,6-dione (7)

To a mixture of tert-butyl (1-(4-((2-(2,6-dioxopiperidin-3-yl)-1-oxoisoindolin-5-yl)amino)butanoyl)piperidin-4-yl)carbamate (260 mg, 492.79 μmol) in dioxane (10 mL) was added HCl/dioxane (4 M, 10 mL) at 20° C., the mixture was stirred at 20° C. for 2 h under N₂ atmosphere. LCMS showed 87% peak with the desired mass and no peak with the starting material. The reaction mixture was concentrated under reduce pressure to afford 3-(5-((4-(4-aminopiperidin-1-yl)-4-oxobutyl)amino)-1-oxoisoindolin-2-yl)piperidine-2,6-dione (245 mg, crude, 2HCl) as a light yellow solid. MS(M+H)⁺=428.3

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1-oxoisoindolin-5-yl)amino)butanoyl)piperidin-4-yl)-2-fluoro-5-methoxybenzamide (Compound 36)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (220 mg, 472.69 μmol) in DMF (5 mL) were added HATU (345.71 mg, 909.22 μmol), DIPEA (305.46 mg, 2.36 mmol, 411.67 μL) and 3-(5-((4-(4-aminopiperidin-1-yl)-4-oxobutyl)amino)-1-oxoisoindolin-2-yl)piperidine-2,6-dione (245 mg, 489.59 μmol, 2HCl) at 25° C. The mixture was stirred at 25° C. for 12 h under N₂ atmosphere. LCMS showed a main peak with the desired mass and no peak with the starting material. To the reaction mixture was added H₂O (20 mL), the mixture was extracted with EtOAc (40 mL×2), the combined organic layers were washed with brine (30 mL×3), dried over Na₂SO₄, filtered and the filtrate was concentrated under reduced pressure. The residue was purified by prep-HPLC (column: Phenomenex luna C₁₈ 150*40 mm*15 μm; mobile phase: [water(HCl)-ACN]; B %: 30%-60%, 11 min; Column Temp: 30° C.) and repurified by prep-HPLC (column: Waters Xbridge C₁₈ 150*50 mm*10 μm; mobile phase: [water(NH₄HCO₃)-ACN]; B %: 30%-60%, 11 min; Column Temp: 30° C.) followed by lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1-oxoisoindolin-5-yl)amino)butanoyl)piperidin-4-yl)-2-fluoro-5-methoxybenzamide (159.4 mg, 178.55 μmol, 37.77% yield, 98% purity) as a white solid. MS(M+H)⁺=875.6

¹H NMR (400 MHz, DMSO-d₆) δ=10.92 (s, 1H), 8.32-8.23 (m, 2H), 8.05-7.95 (m, 2H), 7.39 (d, m, 1H), 7.21 (d, J=6.7 Hz, 1H), 6.71-6.61 (m, 2H), 6.43 (t, J=5.3 Hz, 1H), 5.05-4.95 (m, 1H), 4.87-4.78 (m, 1H), 4.37-4.25 (m, 2H), 4.17-3.99 (m, 4H), 3.92 (s, 3H), 3.90-3.83 (m, 1H), 3.33-3.31 (m, 3H), 3.19-3.09 (m, 3H), 2.94-2.83 (m, 1H), 2.78-2.68 (m, 1H), 2.62-2.55 (m, 1H), 2.47-2.42 (m, 2H), 2.38-2.30 (m, 1H), 2.02-1.93 (m, 3H), 1.88-1.71 (m, 6H), 1.66-1.55 (m, 4H), 1.54-1.36 (m, 2H).

### Example 37. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2-(2,6-dioxopiperidin-3-yl)-1,3-dioxoisoindolin-4-yl)oxy)acetyl)piperidin-4-yl)-2-fluoro-5-methoxybenzamide (Compound 37)

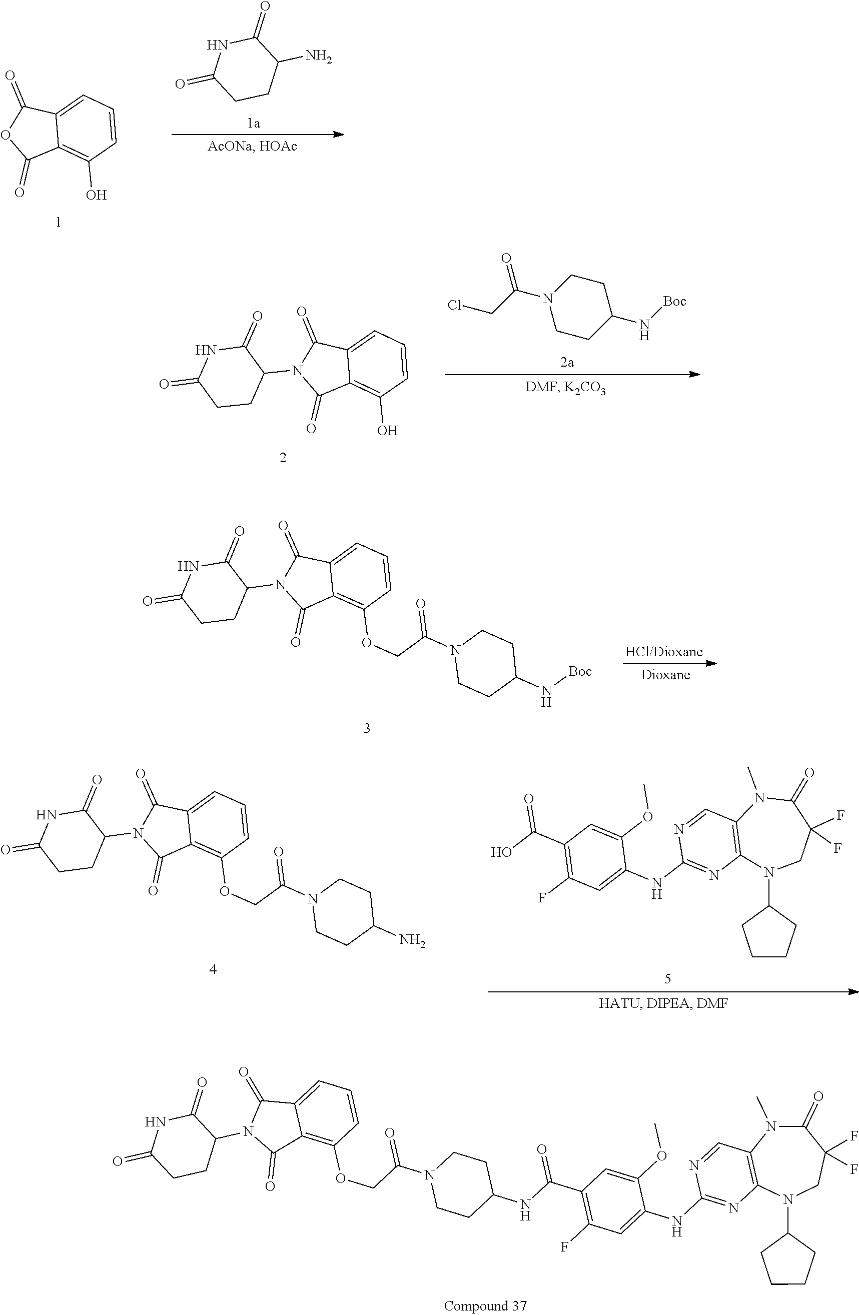

### Step 1. Synthesis of 2-(2,6-dioxopiperidin-3-yl)-4-hydroxyisoindoline-1,3-dione (2)

To a mixture of 4-hydroxyisobenzofuran-1,3-dione (25 g, 152.33 mmol) in CH₃COOH (400 mL) was added 3-aminopiperidine-2,6-dione (27.58 g, 167.57 mmol, HCl) and AcONa (14.99 g, 182.80 mmol, 1.2 eq) in one portion at 25° C. The mixture was stirred at 120° C. for 12 h. LCMS showed the 4-hydroxyisobenzofuran-1,3-dione was consumed completely and a main peak with desired mass. The reaction mixture was filtrated. The cake was washed with H₂O (80 mL×3) and MeOH (60 mL×3). The cake was dried in vacuum to afford 2-(2,6-dioxopiperidin-3-yl)-4-hydroxyisoindoline-1,3-dione (36 g, 131.28 mmol, 86.18% yield, 100% purity) as purple solid, which was used for the next step directly. MS(M+H)⁺=275.1

¹H NMR (400 MHz, DMSO-d₆) δ=11.18 (s, 1H), 11.09 (s, 1H), 7.65 (dd, J=7.3, 8.4 Hz, 1H), 7.32 (d, J=7.0 Hz, 1H), 7.25 (d, J=8.2 Hz, 1H), 5.07 (dd, J=5.4, 12.8 Hz, 1H), 2.96-2.79 (m, 1H), 2.63-2.50 (m, 2H), 2.08-1.96 (m, 1H).

### Step 2. Synthesis of tert-butyl (1-(2-((2-(2,6-dioxopiperidin-3-yl)-1,3-dioxoisoindolin-4-yl)oxy)acetyl)piperidin-4-yl)carbamate (3)

To a solution of 2-(2,6-dioxopiperidin-3-yl)-4-hydroxyisoindoline-1,3-dione (500 mg, 1.82 mmol) in DMF (10 mL) was added K₂CO₃ (377.99 mg, 2.73 mmol) and tert-butyl (1-(2-chloroacetyl)piperidin-4-yl)carbamate (504.61 mg, 1.82 mmol) at 25° C. The mixture was stirred at 25° C. for 1 hr. LCMS showed the 2-(2,6-dioxopiperidin-3-yl)-4-hydroxyisoindoline-1,3-dione was consumed completely, and a peak (41%) with desired mass. The reaction mixture was filtered through a celite pad. The filtrate was purified by prep-HPLC (column: Phenomenex luna C₁₈ 150×40 mm×15 μm; mobile phase: [water(TFA)-ACN]; B %: 22%-52%, 11 min) and lyophilized to afford tert-butyl (1-(2-((2-(2,6-dioxopiperidin-3-yl)-1,3-dioxoisoindolin-4-yl)oxy)acetyl)piperidin-4-yl) carbamate (0.5 g, 874.59 μmol, 47.97% yield, 90% purity) as white solid. MS(M+H)⁺=515.2

### Step 3. Synthesis of 4-(2-(4-aminopiperidin-1-yl)-2-oxoethoxy)-2-(2,6-dioxopiperidin-3-yl)isoindoline-1,3-dione (4)

To a solution of tert-butyl (1-(2-((2-(2,6-dioxopiperidin-3-yl)-1,3-dioxoisoindolin-4-yl)oxy)acetyl)piperidin-4-yl) carbamate (0.5 g, 971.77 μmol) in dioxane (2 mL) was added HCl/dioxane (4 M, 10 mL) at 25° C. The resulting mixture was stirred at 25° C. for 0.5 hr. LCMS showed the starting material was consumed completely, and a main peak with desired mass. The mixture solution was concentrated under reduced pressure to give 4-(2-(4-aminopiperidin-1-yl)-2-oxoethoxy)-2-(2,6-dioxopiperidin-3-yl)isoindoline-1,3-dione (0.4 g, crude, HCl) as white solid, which was used for the next step directly. MS(M+H)⁺=415.0

### Step 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2-(2,6-dioxopiperidin-3-yl)-1,3-dioxoisoindolin-4-yl)oxy)acetyl)piperidin-4-yl)-2-fluoro-5-methoxybenzamide (Compound 37)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (70 mg, 150.40 μmol) in DMF (1 mL) was added HATU (85.78 mg, 225.60 μmol) and DIPEA (58.31 mg, 451.20 μmol, 78.59 μL). The mixture was stirred at 25° C. for 10 min. To mixture was added 4-(2-(4-aminopiperidin-1-yl)-2-oxoethoxy)-2-(2,6-dioxopiperidin-3-yl)isoindoline-1,3-dione (67.81 mg, 150.40 μmol, HCl). The mixture was stirred at 25° C. for 12 h. LCMS showed the 4-(2-(4-aminopiperidin-1-yl)-2-oxoethoxy)-2-(2,6-dioxopiperidin-3-yl)isoindoline-1,3-dione was consumed completely and a main peak with desired mass. The mixture was purified by prep-HPLC (column: Waters Xbridge 150×25 mm×5 μm; mobile phase: [water(NH₄HCO₃)-ACN]; B %: 40%-70%, min) and re-purified by prep-TLC (Dichloromethane:Methanol=20:1; Rf=0.3) to give the crude product. The crude product was purified by prep-HPLC (column: Waters Xbridge 150×25 mm×5 μm; mobile phase: [water(NH₄HCO₃)-ACN]; B %: 39%-69%, 9 min) and lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2-(2,6-dioxopiperidin-3-yl)-1,3-dioxoisoindolin-4-yl)oxy)acetyl)piperidin-4-yl)-2-fluoro-5-methoxybenzamide (46.6 mg, 48.66 μmol, 32.36% yield, 90% purity) as white solid. MS(M+H)⁺=862.1

¹H NMR (400 MHz, DMSO-d₆) δ=11.36-10.82 (m, 1H), 8.35-8.21 (m, 2H), 8.12-7.96 (m, 2H), 7.77 (dd, J=7.5, 8.4 Hz, 1H), 7.45 (d, J=7.2 Hz, 1H), 7.32 (d, J=8.7 Hz, 1H), 7.20 (d, J=6.6 Hz, 1H), 5.32-5.15 (m, 2H), 5.10 (dd, J=5.4, 13.0 Hz, 1H), 4.91-4.74 (m, 1H), 4.31-4.16 (m, 1H), 4.15-3.99 (m, 3H), 3.92 (s, 3H), 3.87-3.74 (m, 1H), 3.33-3.27 (m, 3H), 3.26-3.12 (m, 1H), 2.96-2.78 (m, 2H), 2.64-2.54 (m, 2H), 2.06-1.82 (m, 5H), 1.78-1.33 (m, 8H).

### Example 38. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2-(2,6-dioxopiperidin-3-yl)-1,3-dioxoisoindolin-4-yl)oxy)acetyl)piperidin-4-yl)-3-methoxybenzamide (Compound 38)

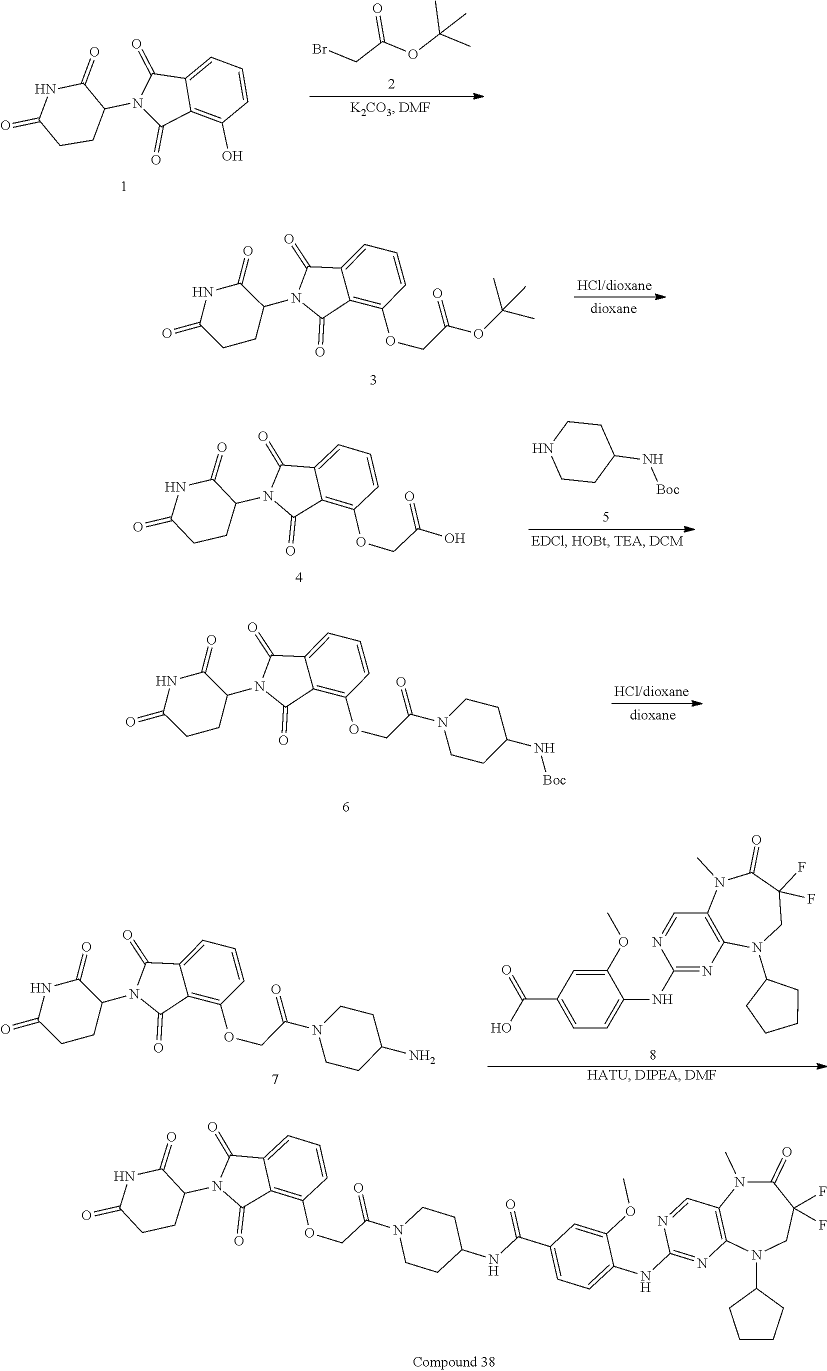

The compound 38 was synthesized by the method described in the scheme similar to the method described in Example 23.

MS(M+H)⁺=844.2, ¹H NMR (400 MHz, DMSO-d₆) δ=11.10 (s, 1H), 8.33-8.24 (m, 2H), 8.21 (d, J=7.6 Hz, 1H), 7.97 (s, 1H), 7.83-7.73 (m, 1H), 7.54-7.42 (m, 3H), 7.33 (d, J=8.6 Hz, 1H), 5.33-5.15 (m, 2H), 5.10 (dd, J=5.3, 12.9 Hz, 1H), 4.84-4.69 (m, 1H), 4.36-4.23 (m, 1H), 4.13-3.99 (m, 3H), 3.94 (s, 3H), 3.91-3.80 (m, 1H), 3.50-3.34 (m, 3H), 3.19 (t, J=11.9 Hz, 1H), 2.95-2.83 (m, 1H), 2.83-2.73 (m, 1H), 2.63-2.55 (m, 2H), 2.07-2.00 (m, 1H), 1.99-1.81 (m, 4H), 1.78-1.67 (m, 2H), 1.66-1.53 (m, 5H), 1.52-1.39 (m, 1H).

### Example 39. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((3R)-1-(2-((2-(2,6-dioxopiperidin-3-yl)-1,3-dioxoisoindolin-4-yl)oxy)acetyl)pyrrolidin-3-yl)-3-methoxybenzamide (Compound 39)

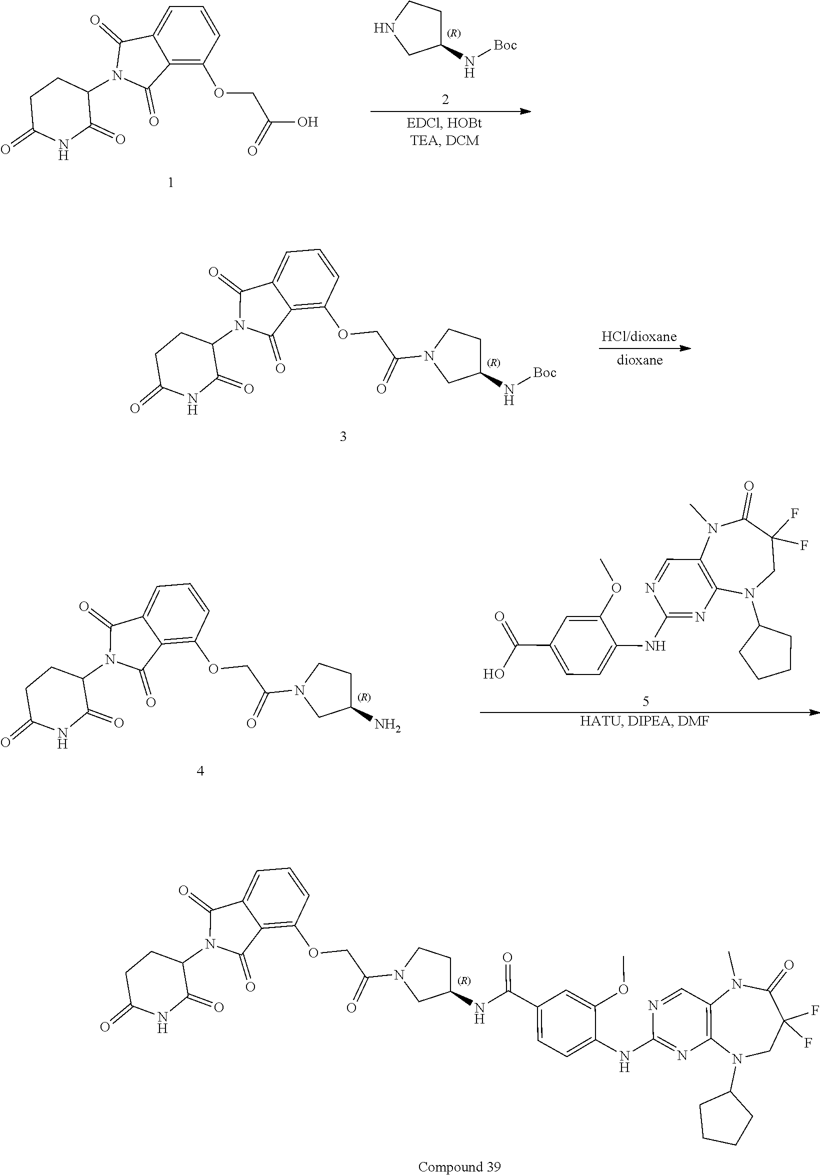

The compound 39 was synthesized by the method described in the scheme similar to the method described in Example 23.

MS (M+H)⁺=830.2, ¹H NMR (400 MHz, DMSO-d6) δ=11.11 (s, 1H), 8.58-8.37 (m, 1H), 8.37-8.22 (m, 2H), 7.99 (d, J=2.7 Hz, 1H), 7.86-7.71 (m, 1H), 7.59-7.48 (m, 2H), 7.45 (dd, J=2.8, 7.1 Hz, 1H), 7.38 (dd, J=4.5, 8.6 Hz, 1H), 5.23-4.99 (m, 3H), 4.83-4.70 (m, 1H), 4.63-4.42 (m, 1H), 4.15-4.01 (m, 2H), 3.95 (s, 4H), 3.79-3.45 (m, 3H), 3.43-3.40 (m, 3H), 2.98-2.81 (m, 1H), 2.62-2.56 (m, 2H), 2.30-2.10 (m, 1H), 2.10-1.90 (m, 4H), 1.70-1.72 (m, 2H), 1.66-1.53 (m, 4H).

### Example 40. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((3S)-1-(2-((2-(2,6-dioxopiperidin-3-yl)-1,3-dioxoisoindolin-4-yl)oxy)acetyl)pyrrolidin-3-yl)-3-methoxybenzamide (Compound 40)

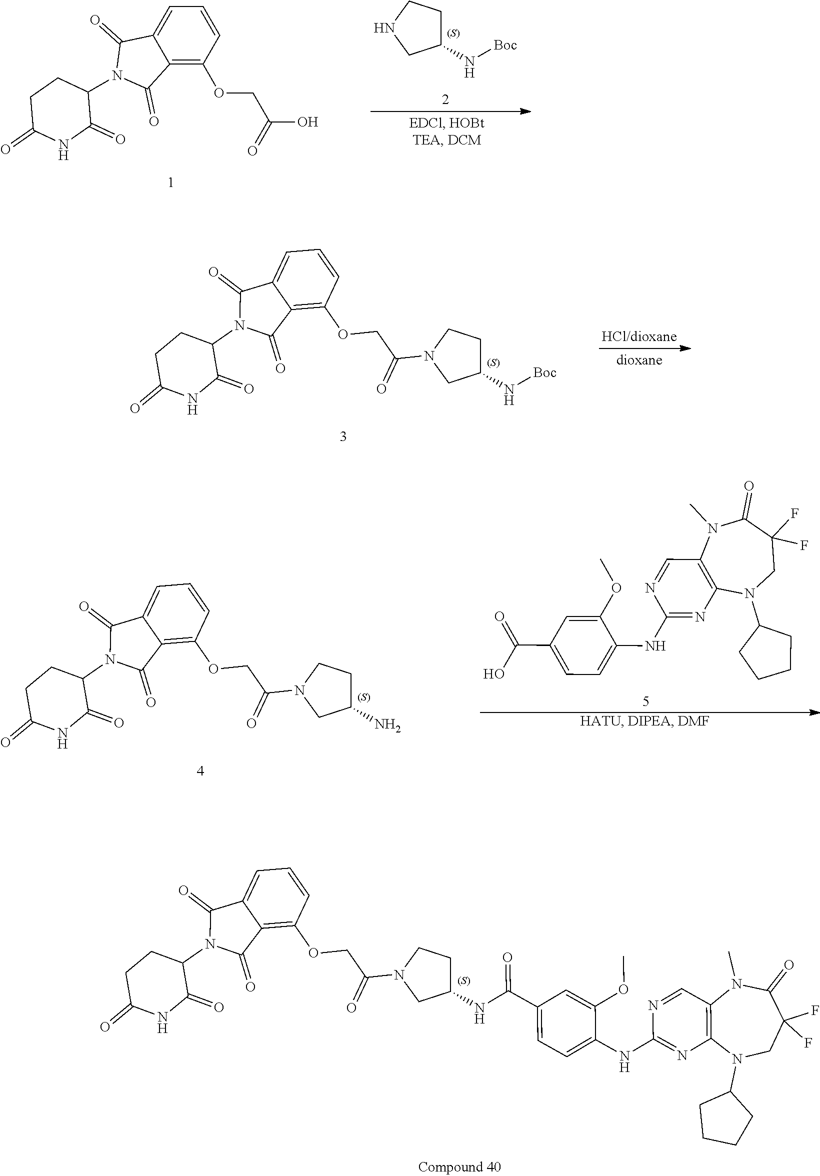

The compound 40 was synthesized by the method described in the scheme similar to the method described in Example 23.

MS(M+H)⁺=830.2, ¹H NMR (400 MHz, DMSO-d₆) δ=11.10 (s, 1H), 8.52-8.36 (m, 1H), 8.34-8.20 (m, 2H), 7.99 (d, J=2.8 Hz, 1H), 7.77 (dt, J=3.5, 7.9 Hz, 1H), 7.56-7.48 (m, 2H), 7.45 (dd, J=2.8, 7.2 Hz, 1H), 7.38 (dd, J=4.4, 8.6 Hz, 1H), 5.17-4.97 (m, 3H), 4.85-4.70 (m, 1H), 4.64-4.41 (m, 1H), 4.05 (t, J=14.1 Hz, 2H), 3.99-3.82 (m, 4H), 3.76-3.51 (m, 2H), 3.48-3.39 (m, 1H), 3.37 (s, 3H), 2.96-2.83 (m, 1H), 2.64-2.53 (m, 2H), 2.29-2.10 (m, 1H), 2.08-1.90 (m, 4H), 1.84-1.72 (m, 2H), 1.65-1.54 (m, 4H).

### Example 41. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2-(2,6-dioxopiperidin-3-yl)-1,3-dioxoisoindolin-4-yl)thio)acetyl)piperidin-4-yl)-2-fluoro-5-methoxybenzamide (Compound 41)

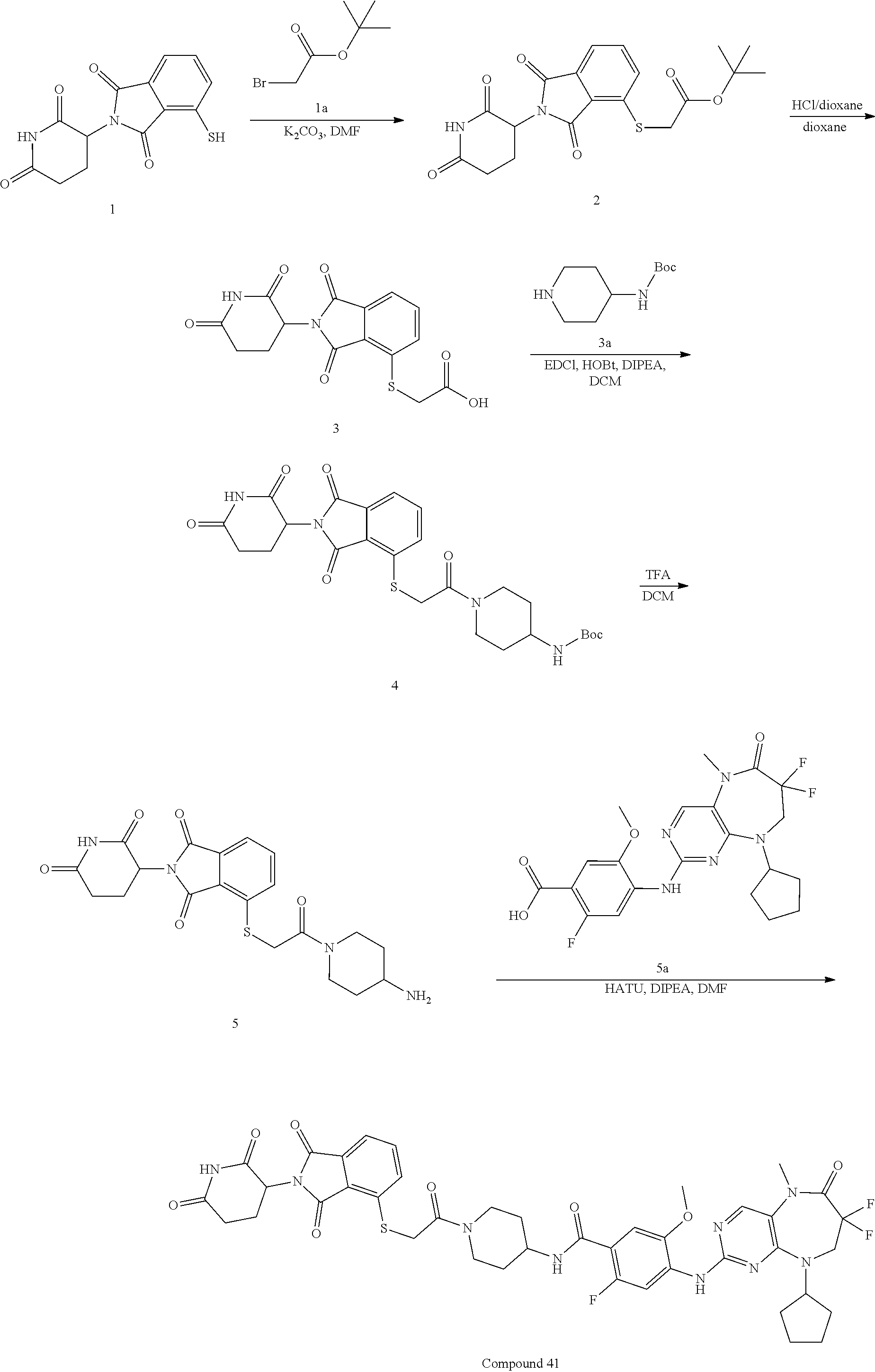

The compound 41 was synthesized by the method described in the scheme similar to the method described in Examples 11 and 23.

MS(M+H)⁺=878.9, ¹H NMR (400 MHz, DMSO-d₆) δ=11.14 (br s, 1H), 8.30 (s, 1H), 8.25 (d, J=13.3 Hz, 1H), 8.08-8.00 (m, 2H), 7.88-7.74 (m, 2H), 7.64 (d, J=6.8 Hz, 1H), 7.20 (d, J=6.6 Hz, 1H), 5.13 (dd, J=5.4, 12.7 Hz, 1H), 4.88-4.75 (m, 1H), 4.37-4.22 (m, 3H), 4.15-3.99 (m, 4H), 3.92 (s, 3H), 3.33 (br s, 3H), 3.29-3.21 (m, 1H), 2.96-2.77 (m, 2H), 2.65-2.52 (m, 2H), 2.10-1.81 (m, 5H), 1.75-1.55 (m, 7H), 1.49-1.33 (m, 1H).

### Example 42. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1,3-dioxoisoindolin-4-yl)thio)butanoyl)piperidin-4-yl)-2-fluoro-5-methoxybenzamide (Compound 42)

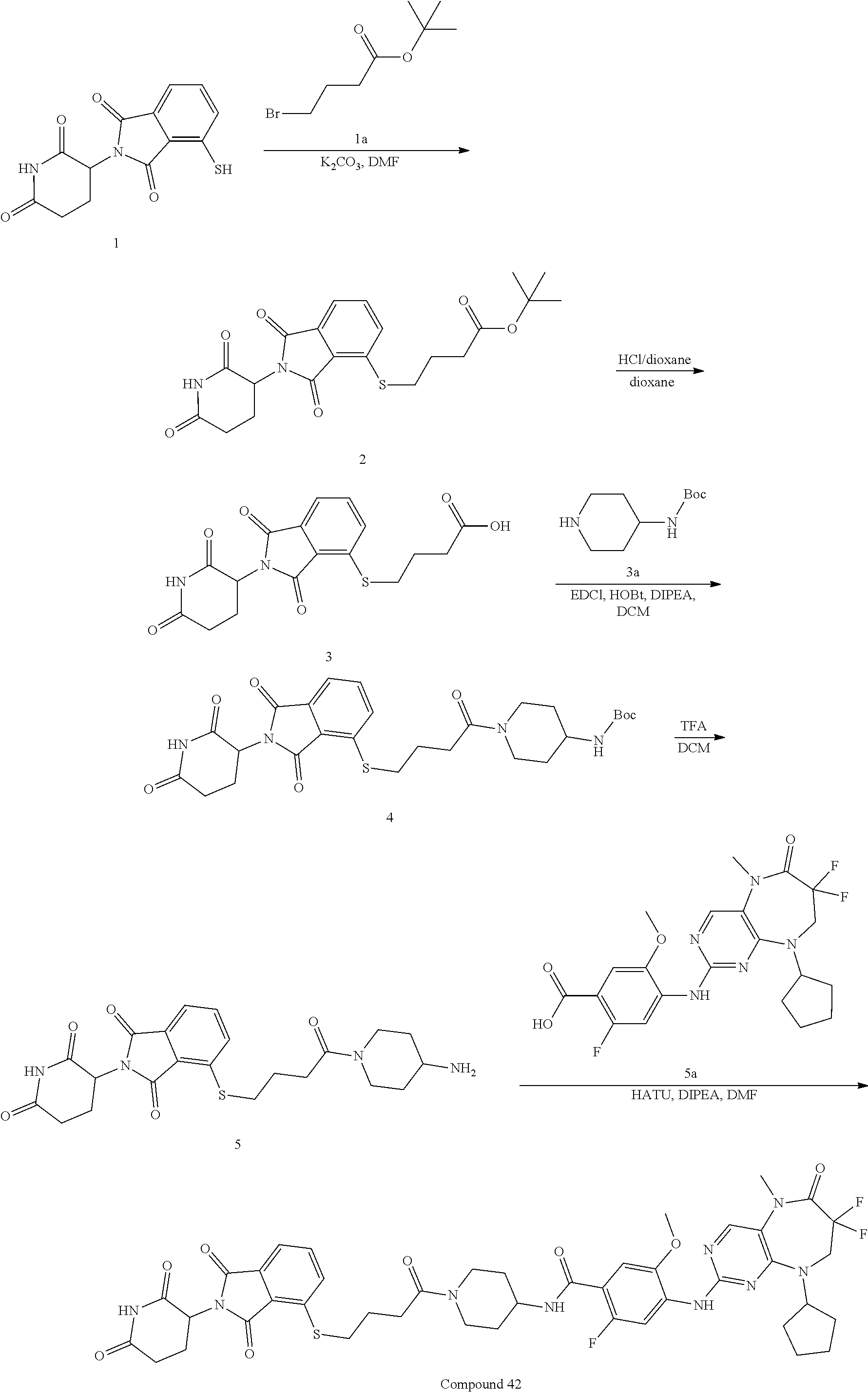

The compound 42 was synthesized by the method described in the scheme similar to the method described in Examples 11 and 23.

MS(M+H)⁺=906.4, ¹H NMR (400 MHz, DMSO-d₆) δ=11.09 (br s, 1H), 8.32-8.21 (m, 2H), 8.04 (s, 1H), 7.96 (dd, J=3.2, 7.7 Hz, 1H), 7.90-7.84 (m, 1H), 7.83-7.76 (m, 1H), 7.63 (d, J=6.9 Hz, 1H), 7.20 (d, J=6.8 Hz, 1H), 5.11 (dd, J=5.4, 12.8 Hz, 1H), 4.88-4.75 (m, 1H), 4.32 (br d, J=12.9 Hz, 1H), 4.14-3.96 (m, 3H), 3.95-3.80 (m, 4H), 3.33 (br s, 3H), 3.22-3.06 (m, 3H), 2.94-2.82 (m, 1H), 2.75 (br t, J=11.3 Hz, 1H), 2.63-2.52 (m, 4H), 2.09-1.79 (m, 7H), 1.76-1.55 (m, 6H), 1.53-1.31 (m, 2H).

### Example 43. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1,3-dioxoisoindolin-4-yl)amino)-4-oxobutanoyl)piperidin-4-yl)-2-fluoro-5-methoxybenzamide (Compound 43)

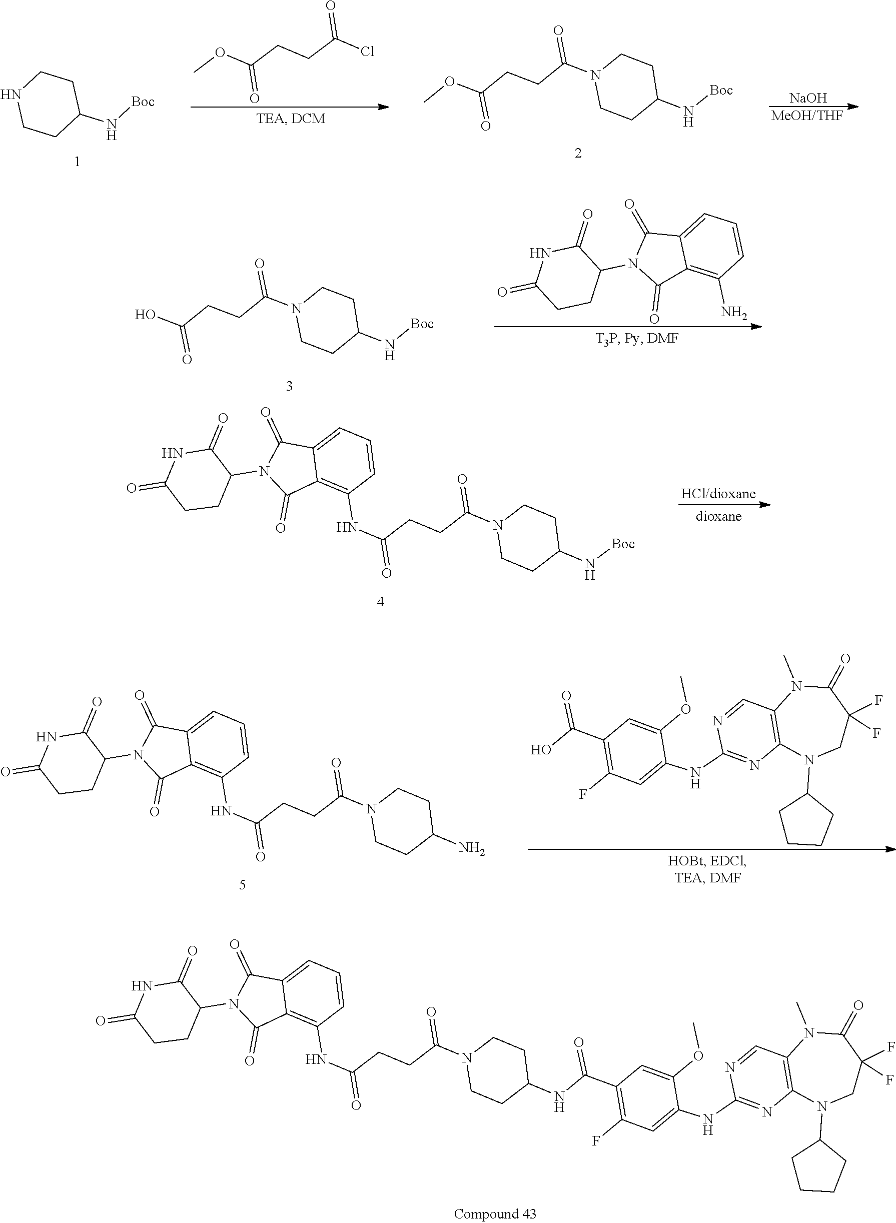

### Step 1. Synthesis of methyl 4-(4-((tert-butoxycarbonyl)amino)piperidin-1-yl)-4-oxobutanoate (2)

To the solution of tert-butyl N-(4-piperidyl) carbamate (2 g, 9.99 mmol) and TEA (3.03 g, 29.96 mmol, 4.17 mL) in DCM (50 mL) was added methyl 4-chloro-4-oxo-butanoate (1.65 g, 10.98 mmol, 1.36 mL) and the resulting mixture was stirred at 20° C. for 12 h. LCMS showed that the reaction was completed. The mixture was poured into water (100 mL) and extracted with DCM (50 mL×3), the combined organic layer was washed with brine (200 mL), dried over Na₂SO₄, filtered and concentrated. The residue was purified by flash silica gel chromatography (45 g SepaFlash® Silica Flash Column, Eluent of 0˜50% EtOAc/Petroleum ether gradient @80 mL/min) to afford methyl 4-(4-((tert-butoxycarbonyl)amino)piperidin-1-yl)-4-oxobutanoate (2.7 g, 7.13 mmol, 71.38% yield, 83% purity) as yellow solid. MS(M+H)⁺=315.1

### Step 2. Synthesis of 4-(4-((tert-butoxycarbonyl)amino)piperidin-1-yl)-4-oxobutanoic acid (3)

To the solution of methyl 4-(4-((tert-butoxycarbonyl)amino)piperidin-1-yl)-4-oxobutanoate (2.5 g, 7.95 mmol) in THF (10 mL) and MeOH (10 mL) was added a solution of NaOH (1.59 g, 39.76 mmol) in H₂O (10 mL) and the resulting mixture was stirred at 20° C. for 12 h. LCMS showed that the reaction was completed. The organic solvent was concentrated and the residue was adjusted pH=6 by 1 M HCl, the resulting mixture was extracted with EtOAc (50 mL×3), the combined organic layer was washed with brine (100 mL×2), dried over Na₂SO₄, filtered and concentrated to afford 4-(4-((tert-butoxycarbonyl)amino)piperidin-1-yl)-4-oxobutanoic acid (1.6 g, 5.33 mmol, 66.99% yield) as yellow solid. MS(M+H)⁺=301.1

### Step 3. Synthesis of tert-butyl (1-(4-((2-(2,6-dioxopiperidin-3-yl)-1,3-dioxoisoindolin-4-yl)amino)-4-oxobutanoyl) piperidin-4-yl)carbamate (4)

To the solution of 4-(4-((tert-butoxycarbonyl)amino)piperidin-1-yl)-4-oxobutanoic acid (1 g, 3.33 mmol) and 4-amino-2-(2,6-dioxo-3-piperidyl) isoindoline-1,3-dione (1.36 g, 4.99 mmol) in DMF (20 mL) was added T₃P (12.71 g, 19.98 mmol, 11.88 mL, 50% purity) and Py (2.63 g, 33.29 mmol, 2.69 mL) and the resulting mixture was stirred at 80° C. for 12 h. TLC (Petroleum ether/EtOAc=1/1) showed that the reaction was completed. The mixture was poured into water (100 mL) and extracted with EtOAc (50 mL×3), the combined organic layer was washed with brine (100 mL), dried over Na₂SO₄, filtered and concentrated. The residue was purified by flash silica gel chromatography (10 g SepaFlash® Silica Flash Column, Eluent of 0˜50% EtOAc/Petroleum ether gradient @50 mL/min) to afford tert-butyl (1-(4-((2-(2,6-dioxopiperidin-3-yl)-1,3-dioxoisoindolin-4-yl)amino)-4-oxobutanoyl)piperidin-4-yl)carbamate (0.7 g, 1.26 mmol, 37.84% yield) as yellow oil. MS(M+H)⁺=556.2

### Step 4. Synthesis of 4-(4-aminopiperidin-1-yl)-N-(2-(2,6-dioxopiperidin-3-yl)-1,3-dioxoisoindolin-4-yl)-4-oxobutanamide (5)

To the solution of tert-butyl (1-(4-((2-(2,6-dioxopiperidin-3-yl)-1,3-dioxoisoindolin-4-yl)amino)-4-oxobutanoyl)piperidin-4-yl)carbamate (0.7 g, 1.26 mmol) in dioxane (5 mL) was added HCl/dioxane (4 M, 5 mL) and the mixture was stirred at 25° C. for 1 h. LCMS showed that the reaction was completed, the mixture was concentrated to afford 4-(4-aminopiperidin-1-yl)-N-(2-(2,6-dioxopiperidin-3-yl)-1,3-dioxoisoindolin-4-yl)-4-oxobutanamide (0.7 g, crude, HCl) as yellow oil. MS(M+H)⁺=456.0

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1,3-dioxoisoindolin-4-yl)amino)-4-oxobutanoyl)piperidin-4-yl)-2-fluoro-5-methoxybenzamide (Compound 43)

To the solution of 4-(4-aminopiperidin-1-yl)-N-(2-(2,6-dioxopiperidin-3-yl)-1,3-dioxoisoindolin-4-yl)-4-oxobutanamide (300 mg, 609.85 μmol HCl) and 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (283.84 mg, 609.85 μmol) in DMF (10 mL) was added HOBt (123.61 mg, 914.78 μmol), EDCI (175.36 mg, 914.78 μmol) and TEA (185.13 mg, 1.83 mmol, 254.65 μL) and the resulting mixture was stirred at 25° C. for 12 h. LCMS showed the reaction was completed, the mixture was poured into water (20 mL) and extracted with EtOAc (20 mL×3), and the combined organic layer was washed with brine (50 mL), dried over Na₂SO₄, filtered and concentrated in vacuo. The residue was purified by prep-TLC (EtOAc/Methanol=10/1) and re-purified by prep-HPLC (column: Waters Xbridge 150*25 mm*5 μm; mobile phase: [water(NH₄HCO₃)-ACN]; B %: 43%-73%, 10 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-((2-(2,6-dioxopiperidin-3-yl)-1,3-dioxoisoindolin-4-yl)amino)-4-oxobutanoyl)piperidin-4-yl)-2-fluoro-5-methoxybenzamide (67.9 mg, 73.70 μmol, 12.08% yield, 98% purity) as white solid. MS(M+H)⁺=903.0

¹H NMR (DMSO-d₆) δ=11.15 (br s, 1H), 9.76 (s, 1H), 8.51 (d, J=8.4 Hz, 1H), 8.34-8.22 (m, 2H), 8.07-7.97 (m, 2H), 7.89-7.78 (m, 1H), 7.61 (d, J=7.3 Hz, 1H), 7.21 (d, J=6.6 Hz, 1H), 5.16 (dd, J=5.4, 12.9 Hz, 1H), 4.89-4.78 (m, 1H), 4.29 (d, J=12.6 Hz, 1H), 4.14-4.00 (m, 3H), 3.95-3.91 (m, 4H), 3.34 (s, 3H), 3.18 (t, J=11.6 Hz, 1H), 2.97-2.86 (m, 1H), 2.82-2.54 (m, 8H), 2.11-1.89 (m, 4H), 1.82-1.61 (m, 6H), 1.53-1.33 (m, 2H).

### Example 44. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-(2-(2,6-dioxopiperidin-3-yl)-1,3-dioxoisoindolin-4-yl)pyrrolidin-3-yl)acetyl)piperidin-4-yl)-2-fluoro-5-methoxybenzamide (Compound 44)

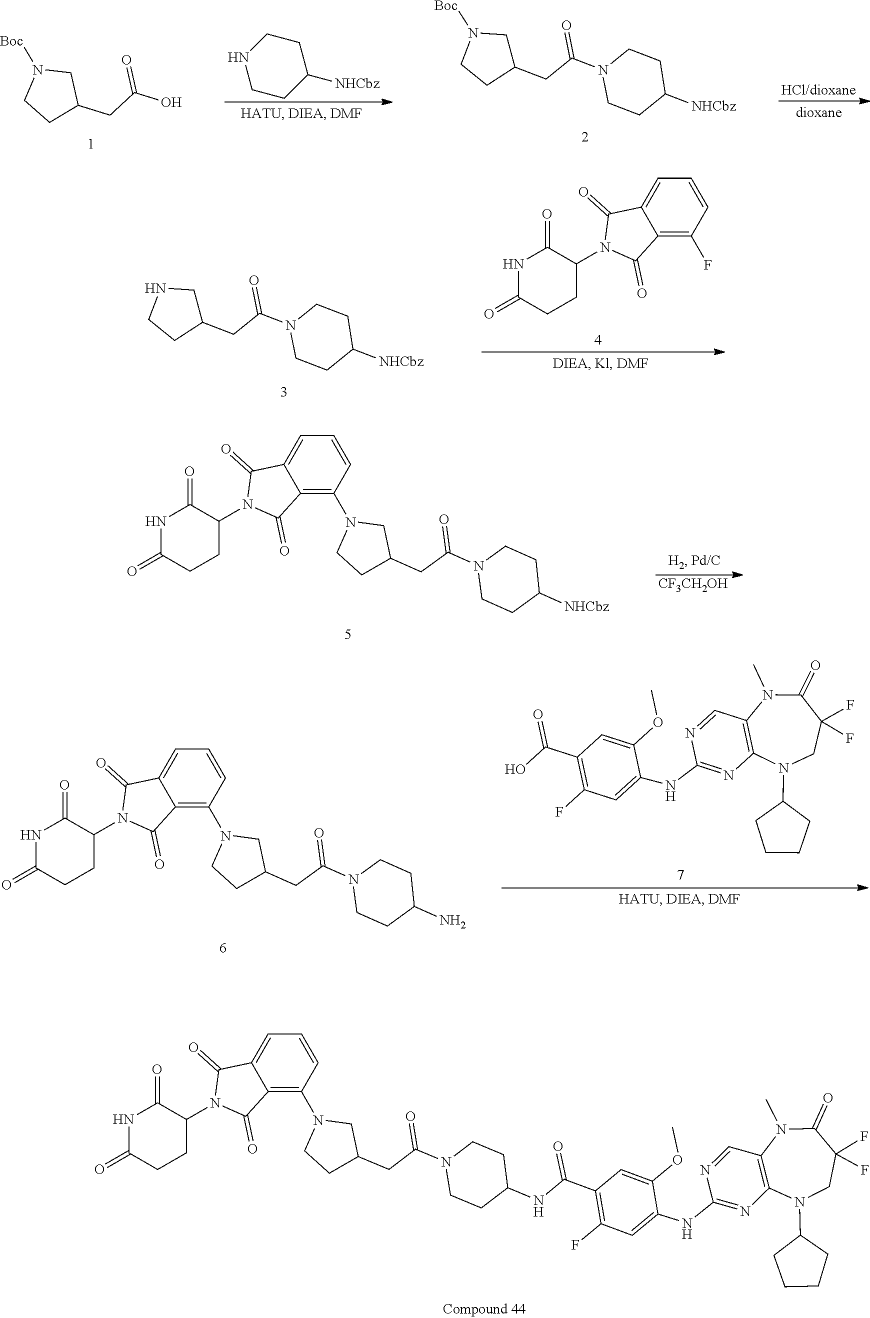

### Step 1. Synthesis of tert-butyl 3-(2-(4-(((benzyloxy)carbonyl)amino)piperidin-1-yl)-2-oxoethyl) pyrrolidine-1-carboxylate (2)

To a solution of 2-(1-(tert-butoxycarbonyl)pyrrolidin-3-yl)acetic acid (500 mg, 2.18 mmol) and benzyl piperidin-4-ylcarbamate (510.95 mg, 2.18 mmol) in DMF (10 mL) were added HATU (1.24 g, 3.27 mmol) and DIEA (845.57 mg, 6.54 mmol, 1.14 mL). The mixture was stirred at 25° C. for 2 h. TLC (Petroleum ether:EtOAc=1:1) indicated 2-(1-(tert-butoxycarbonyl)pyrrolidin-3-yl)acetic acid was consumed completely and one new spot was formed. The reaction mixture was diluted with water (100 mL) and extracted with EtOAc (30 mL×3). The combined organic layers were washed with brine (30 mL), dried over anhydrous sodium sulfate, filtered and concentrated under reduced pressure. The residue was purified by column chromatography (SiO₂, Petroleum ether/EtOAc=2/1 to 1/1) to afford tert-butyl 3-(2-(4-(((benzyloxy)carbonyl)amino)piperidin-1-yl)-2-oxoethyl)pyrrolidine-1-carboxylate (830 mg, 1.79 mmol, 82.00% yield, 96% purity) as light red gum. MS(M+H)⁺=446.6.

### Step 2. Synthesis of benzyl (1-(2-(pyrrolidin-3-yl)acetyl)piperidin-4-yl)carbamate (3)

A mixture of tert-butyl 3-(2-(4-(((benzyloxy)carbonyl)amino)piperidin-1-yl)-2-oxoethyl)pyrrolidine-1-carboxylate (830 mg, 1.86 mmol) and HCl/dioxane (4 M, 5 mL) in DCM (5 mL) was stirred at 25° C. for 1 h. LCMS showed the starting material was consumed completely and one main peak with desired mass was detected. The reaction mixture was concentrated under reduced pressure to afford benzyl (1-(2-(pyrrolidin-3-yl)acetyl)piperidin-4-yl)carbamate (800 mg, crude, HCl salt) as a light yellow solid. MS(M+H)⁺=346.2.

### Step 3. Synthesis of benzyl (1-(2-(1-(2-(2,6-dioxopiperidin-3-yl)-1,3-dioxoisoindolin-4-yl)pyrrolidin-3-yl)acetyl)piperidin-4-yl)carbamate (5)

To a solution of benzyl (1-(2-(pyrrolidin-3-yl)acetyl)piperidin-4-yl)carbamate (300 mg, 868.47 μmol) and 2-(2,6-dioxopiperidin-3-yl)-4-fluoroisoindoline-1,3-dione (239.89 mg, 868.47 μmol) in DMF (6 mL) were added KI (144.17 mg, 868.47 μmol) and DIEA (336.73 mg, 2.61 mmol, 453.82 μL). The mixture was stirred at 80° C. for 16 h. LCMS showed ˜66% of 2-(2,6-dioxopiperidin-3-yl)-4-fluoroisoindoline-1,3-dione was remained and ˜27% desired mass was detected. The reaction mixture was diluted with H₂O (50 mL) and extracted with EtOAc (20 mL×3). The combined organic layers were washed with brine (20 mL), dried over anhydrous sodium sulfate, filtered and concentrated under reduced pressure. The residue was purified by column chromatography (SiO₂, Petroleum ether/EtOAc=1/0 to 0/1) to afford benzyl (1-(2-(1-(2-(2,6-dioxopiperidin-3-yl)-1,3-dioxoisoindolin-4-yl)pyrrolidin-3-yl)acetyl)piperidin-4-yl)carbamate (250 mg, 411.37 μmol, 47.37% yield, 99% purity) as a yellow solid. MS(M+H)⁺=602.1

### Step 4. Synthesis of 4-(3-(2-(4-aminopiperidin-1-yl)-2-oxoethyl)pyrrolidin-1-yl)-2-(2,6-dioxopiperidin-3-yl)isoindoline-1,3-dione (6)

To a solution of benzyl (1-(2-(1-(2-(2,6-dioxopiperidin-3-yl)-1,3-dioxoisoindolin-4-yl)pyrrolidin-3-yl)acetyl)piperidin-4-yl)carbamate (100 mg, 166.21 μmol) in CF₃CH₂OH (4 mL) was added Pd/C (10% purity, 20 mg) under N₂ atmosphere. The suspension was degassed and purged with H₂ for 3 times. The mixture was stirred under H₂ (15 Psi) at 25° C. for 4 h. LCMS showed the starting material was consumed completely and one main peak with desired mass was detected. The reactipon mixture was filtered and filter cake was washed with CF₃CH₂OH (50 mL), the filtrate was concentrated in vacuo to afford 4-(3-(2-(4-aminopiperidin-1-yl)-2-oxoethyl)pyrrolidin-1-yl)-2-(2,6-dioxopiperidin-3-yl)isoindoline-1,3-dione (80 mg, 155.72 μmol, 93.69% yield, 91% purity) as a yellow solid. MS(M+H)⁺=468.1

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-(2-(2,6-dioxopiperidin-3-yl)-1,3-dioxoisoindolin-4-yl)pyrrolidin-3-yl)acetyl)piperidin-4-yl)-2-fluoro-5-methoxybenzamide (Compound 44)

To a solution of 4-(3-(2-(4-aminopiperidin-1-yl)-2-oxoethyl)pyrrolidin-1-yl)-2-(2,6-dioxopiperidin-3-yl)isoindoline-1,3-dione (80 mg, 171.12 μmol) and 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (79.64 mg, 171.12 μmol) in DMF (5 mL) were added HATU (97.60 mg, 256.68 μmol) and DIEA (66.35 mg, 513.35 μmol, 89.42 μL). The mixture was stirred at 25° C. for 2 h. LCMS showed 4-(3-(2-(4-aminopiperidin-1-yl)-2-oxoethyl)pyrrolidin-1-yl)-2-(2,6-dioxopiperidin-3-yl)isoindoline-1,3-dione was consumed completely, ˜72% desired mass was detected. The reaction mixture was diluted with H₂O (50 mL) and extracted with EtOAc (20 mL×3). The combined organic layers were washed with brine (20 mL), dried over anhydrous sodium sulfate, filtered and concentrated under reduced pressure. The residue was purified by prep-HPLC (column: Phenomenex luna C18150×25 mm×10 μm; mobile phase: [water(FA)-ACN]; B %: 47%-77%, 10 min) followed by prep-HPLC (column: Waters Xbridge 150×25 mm×5 μm; mobile phase: [water(NH₄HCO₃)-ACN]; B %: 44%-77%, 10 min). Then the impure product was re-purified by prep-TLC (SiO₂, DCM:MeOH=10:1) and prep-HPLC (column: Waters Xbridge 150×25 mm×5 μm; mobile phase: [water(NH₄HCO₃)-ACN]; B %: 45%-75%, 10 min) to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-(2-(2,6-dioxopiperidin-3-yl)-1,3-dioxoisoindolin-4-yl)pyrrolidin-3-yl)acetyl)piperidin-4-yl)-2-fluoro-5-methoxybenzamide (23.6 mg, 25.79 μmol, 15.07% yield) as a yellow solid. MS(M+H)⁺=915.5 ¹H NMR (400 MHz, DMSO-d₆) δ=11.04 (br s, 1H), 8.33-8.21 (m, 2H), 8.05 (s, 1H), 7.97-7.93 (m, 1H), 7.57 (dd, J=7.1, 8.4 Hz, 1H), 7.21 (d, J=6.6 Hz, 1H), 7.16-7.04 (m, 2H), 5.11-5.04 (m, 1H), 4.88-4.78 (m, 1H), 4.38-4.26 (m, 1H), 4.14-3.99 (m, 3H), 3.92 (s, 3H), 3.90-3.85 (m, 1H), 3.72-3.59 (m, 2H), 3.58-3.50 (m, 1H), 3.34 (s, 3H), 3.18-3.10 (m, 1H), 2.93-2.82 (m, 1H), 2.81-2.72 (m, 1H), 2.64-2.54 (m, 5H), 2.21-2.12 (m, 1H), 2.06-1.93 (m, 3H), 1.91-1.79 (m, 2H), 1.78-1.55 (m, 8H), 1.55-1.34 (m, 2H).

### Example 45. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-(2-(2,6-dioxopiperidin-3-yl)-1,3-dioxoisoindolin-5-yl)pyrrolidin-3-yl)acetyl)piperidin-4-yl)-2-fluoro-5-methoxybenzamide (Compound 45)

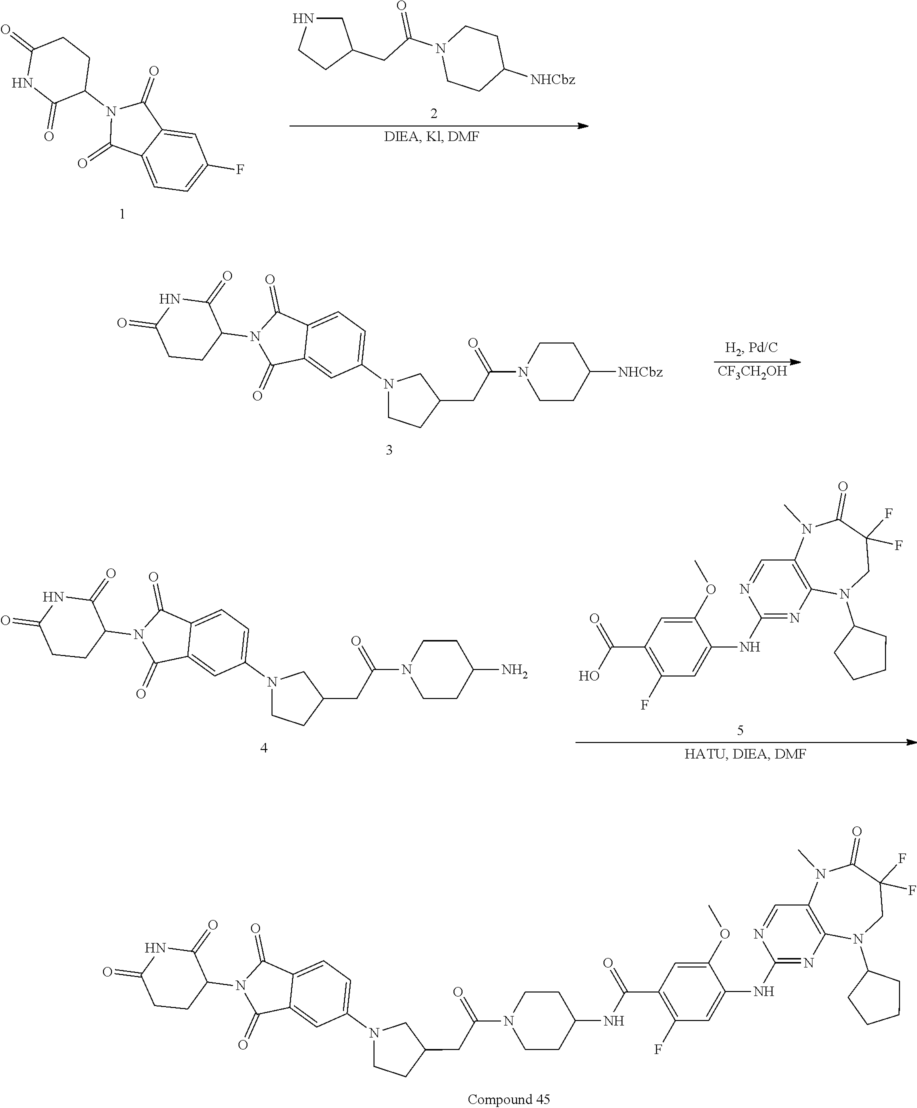

The compound 45 was synthesized by the method described in the scheme similar to the method described in Example 44.

MS(M+H)⁺=915.5, ¹H NMR (400 MHz, DMSO-d₆) δ=11.07-11.02 (m, 1H), 8.31-8.21 (m, 2H), 8.06 (s, 1H), 8.02-7.96 (m, 1H), 7.64 (d, J=8.5 Hz, 1H), 7.20 (d, J=6.6 Hz, 1H), 6.88 (d, J=1.8 Hz, 1H), 6.80 (d, J=1.9, 8.6 Hz, 1H), 5.05 (dd, J=5.4, 12.9 Hz, 1H), 4.88-4.77 (m, 1H), 4.39-4.29 (m, 1H), 4.12-3.99 (m, 3H), 3.92 (s, 3H), 3.90-3.83 (m, 1H), 3.68-3.61 (m, 1H), 3.33 (s, 3H), 3.19-3.05 (m, 2H), 2.94-2.73 (m, 3H), 2.70-2.54 (m, 5H), 2.27-2.16 (m, 1H), 2.04-1.81 (m, 5H), 1.79-1.56 (m, 8H), 1.52-1.35 (m, 2H).

### Example 46. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-((1-(2-(2,6-dioxopiperidin-3-yl)-1,3-dioxoisoindolin-4-yl)pyrrolidin-3-yl)methyl)piperidin-4-yl)-3-methoxybenzamide (Compound 46)

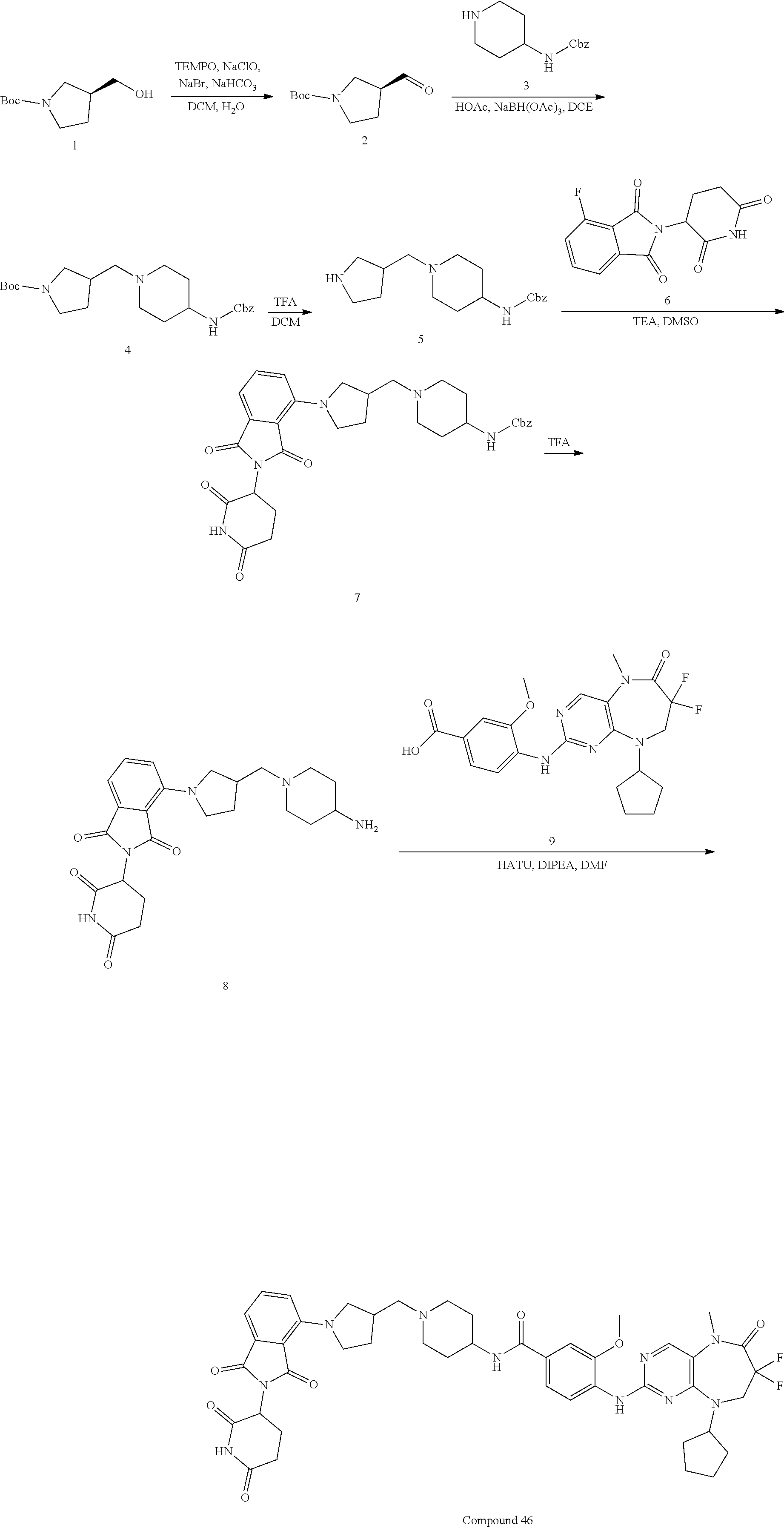

The compound 46 was synthesized by the method described in the scheme similar to the method described in Example 44.

MS(M+H)⁺=869.5, ¹H NMR (400 MHz, DMSO-d₆) δ=11.05 (s, 1H), 8.27-8.25 (m, 2H), 8.10 (d, J=8.8 Hz, 1H), 7.96 (s, 1H), 7.58-7.54 (m, 1H), 7.49-7.47 (m, 2H), 7.13-7.08 (m, 2H), 5.08-5.04 (m, 1H), 4.78-4.74 (m, 1H), 4.04 (br t, J=14.0 Hz, 2H), 3.93 (s, 3H), 3.82-3.74 (m, 1H), 3.65-3.52 (m, 4H), 3.43-3.40 (m, 1H), 3.28 (s, 3H), 2.93-2.83 (m, 3H), 2.59-2.54 (m, 3H), 2.34-2.32 (m, 2H), 2.07-1.91 (m, 6H), 1.79-1.58 (m, 10H).

### Example 47. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(((3S)-1-(2-(2,6-dioxopiperidin-3-yl)-1,3-dioxoisoindolin-4-yl)pyrrolidin-3-yl)methyl)piperidin-4-yl)-3-methoxybenzamide (Compound 47)

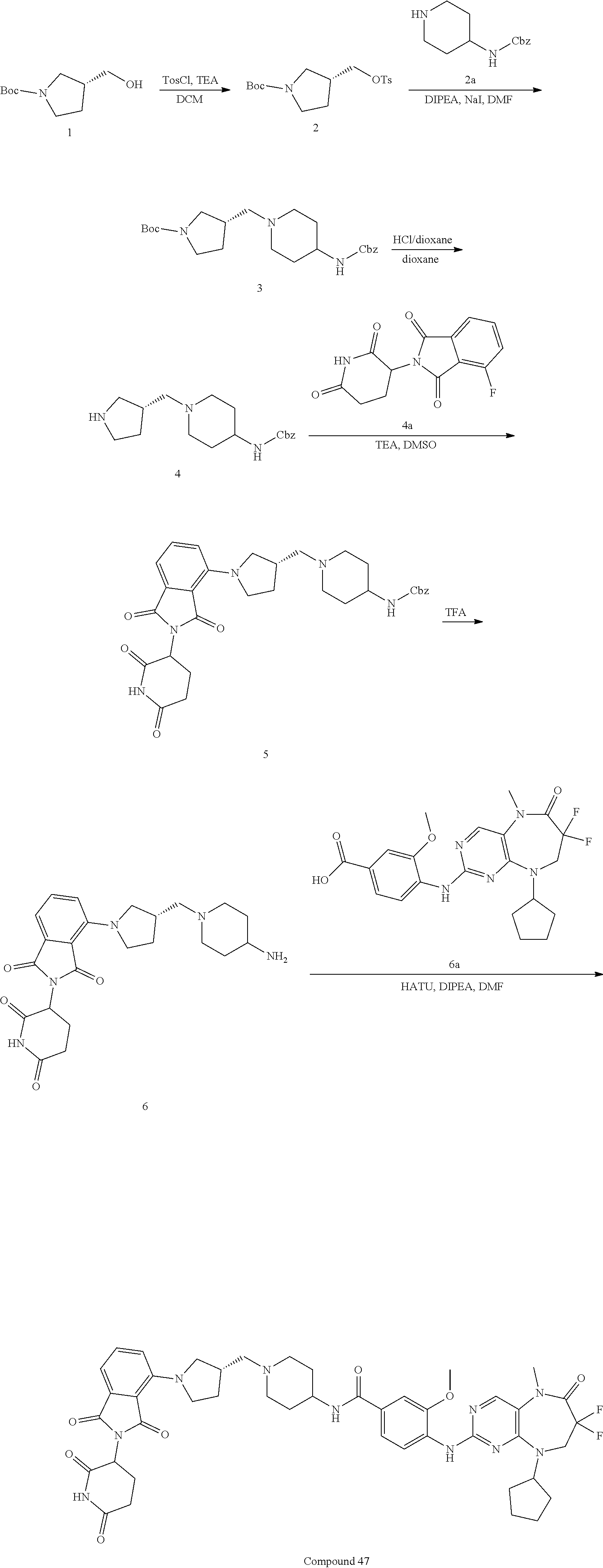

The compound 47 was synthesized by the method described in the scheme similar to the method described in Example 44.

MS(M+H)⁺=869.3, ¹H NMR (400 MHz, DMSO-d₆) δ=11.05 (s, 1H), 8.31-8.22 (m, 2H), 8.15-8.05 (m, 1H), 7.96 (s, 1H), 7.56 (t, J=7.8 Hz, 1H), 7.52-7.43 (m, 2H), 7.11 (br t, J=8.4 Hz, 2H), 5.06 (dd, J=5.5, 12.8 Hz, 1H), 4.83-4.70 (m, 1H), 4.04 (br t, J=14.1 Hz, 2H), 3.93 (s, 3H), 3.86-3.71 (m, 1H), 3.68-3.39 (m, 5H), 3.39-3.35 (m, 3H), 3.03-2.80 (m, 3H), 2.64-2.53 (m, 3H), 2.39-2.31 (m, 2H), 2.13-1.85 (m, 6H), 1.81-1.53 (m, 10H).

### Example 48. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(((3S)-1-(2-(2,6-dioxopiperidin-3-yl)-1,3-dioxoisoindolin-5-yl)pyrrolidin-3-yl)methyl)piperidin-4-yl)-3-methoxybenzamide (Compound 48)

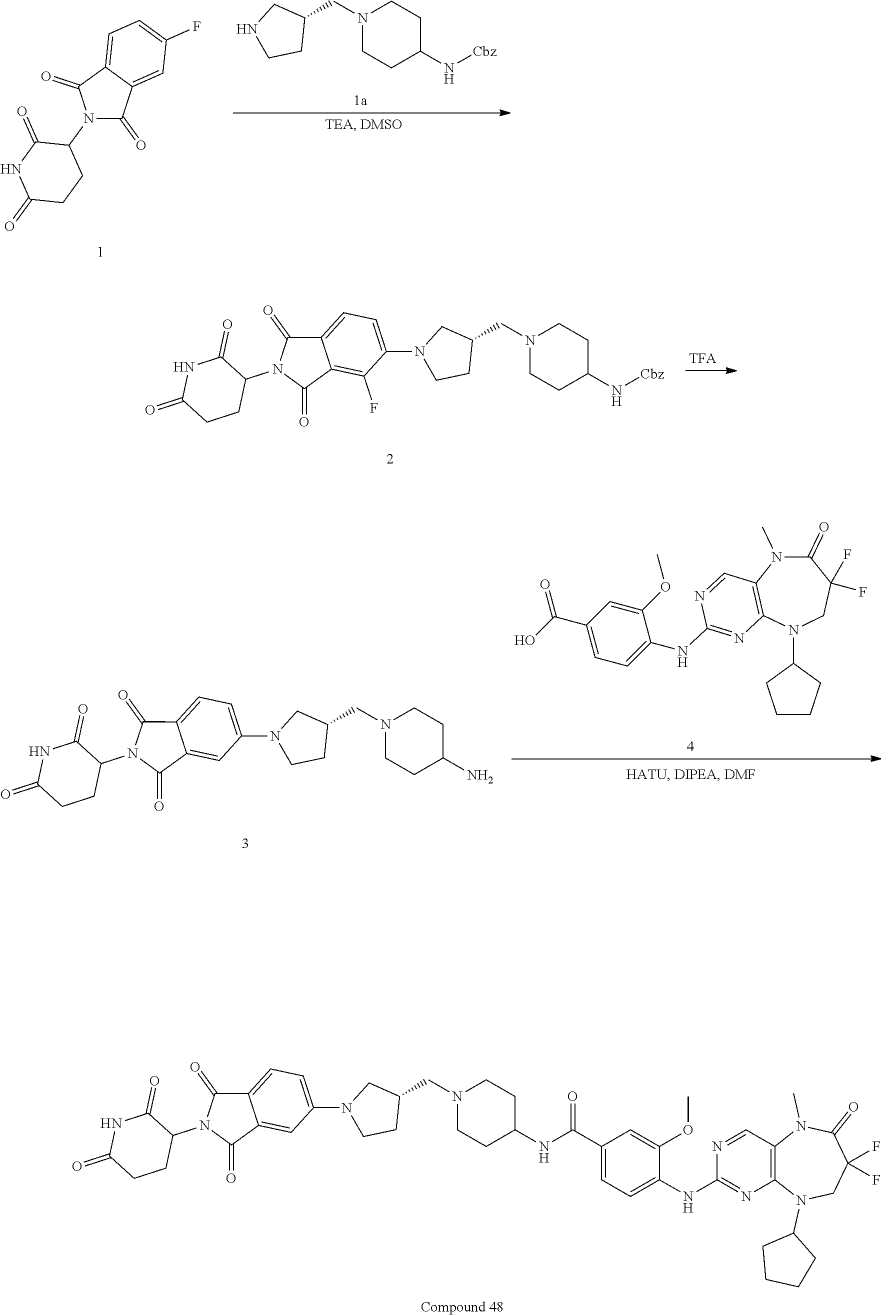 [SMILES: O=C1CCC(N2C(=O)c3ccc(F)cc3C2=O)C(=O)N1 ; O=C(NC1CCN(C[C@@H]2CCNC2)CC1)OCc1ccccc1 ; O=C1CCC(N2C(=O)c3ccc(N4CC[C@@H](CN5CCC(NC(=O)OCc6ccccc6)CC5)C4)c(F)c3C2=O)C(=O)N1 ; NC1CCN(C[C@@H]2CCN(c3ccc4c(c3)C(=O)N(C3CCC(=O)NC3=O)C4=O)C2)CC1 ; COc1cc(C(=O)O)ccc1Nc1ncc2c(n1)N(C1CCCC1)CC(F)(F)C(=O)N2C ; COc1cc(C(=O)NC2CCN(C[C@@H]3CCN(c4ccc5c(c4)C(=O)N(C4CCC(=O)NC4=O)C5=O)C3)CC2)ccc1Nc1ncc2c(n1)N(C1CCCC1)CC(F)(F)C(=O)N2C]

The compound 48 was synthesized by the method described in the scheme similar to the method described in Example 44.

MS(M+H)⁺=869.5, ¹H NMR (400 MHz, CDCl₃) δ=8.49 (d, J=8.4 Hz, 1H), 8.28-8.15 (m, 1H), 8.06 (s, 1H), 7.77 (s, 1H), 7.66 (d, J=8.4 Hz, 1H), 7.45 (d, J=1.6 Hz, 1H), 7.36-7.28 (m, 2H), 6.95 (d, J=1.8 Hz, 1H), 6.73-6.66 (m, 1H), 5.01-4.89 (m, 1H), 4.89-4.77 (m, 1H), 4.23-4.07 (m, 1H), 3.99 (s, 3H), 3.94-3.84 (m, 2H), 3.71-3.59 (m, 1H), 3.56-3.43 (m, 2H), 3.41 (s, 3H), 3.28-3.09 (m, 2H), 2.94-2.68 (m, 5H), 2.44-2.24 (m, 3H), 2.20-2.03 (m, 6H), 1.96-1.68 (m, 10H).

### Example 49. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(((3R)-1-(2-(2,6-dioxopiperidin-3-yl)-1,3-dioxoisoindolin-5-yl)pyrrolidin-3-yl)methyl)piperidin-4-yl)-3-methoxybenzamide (Compound 49)

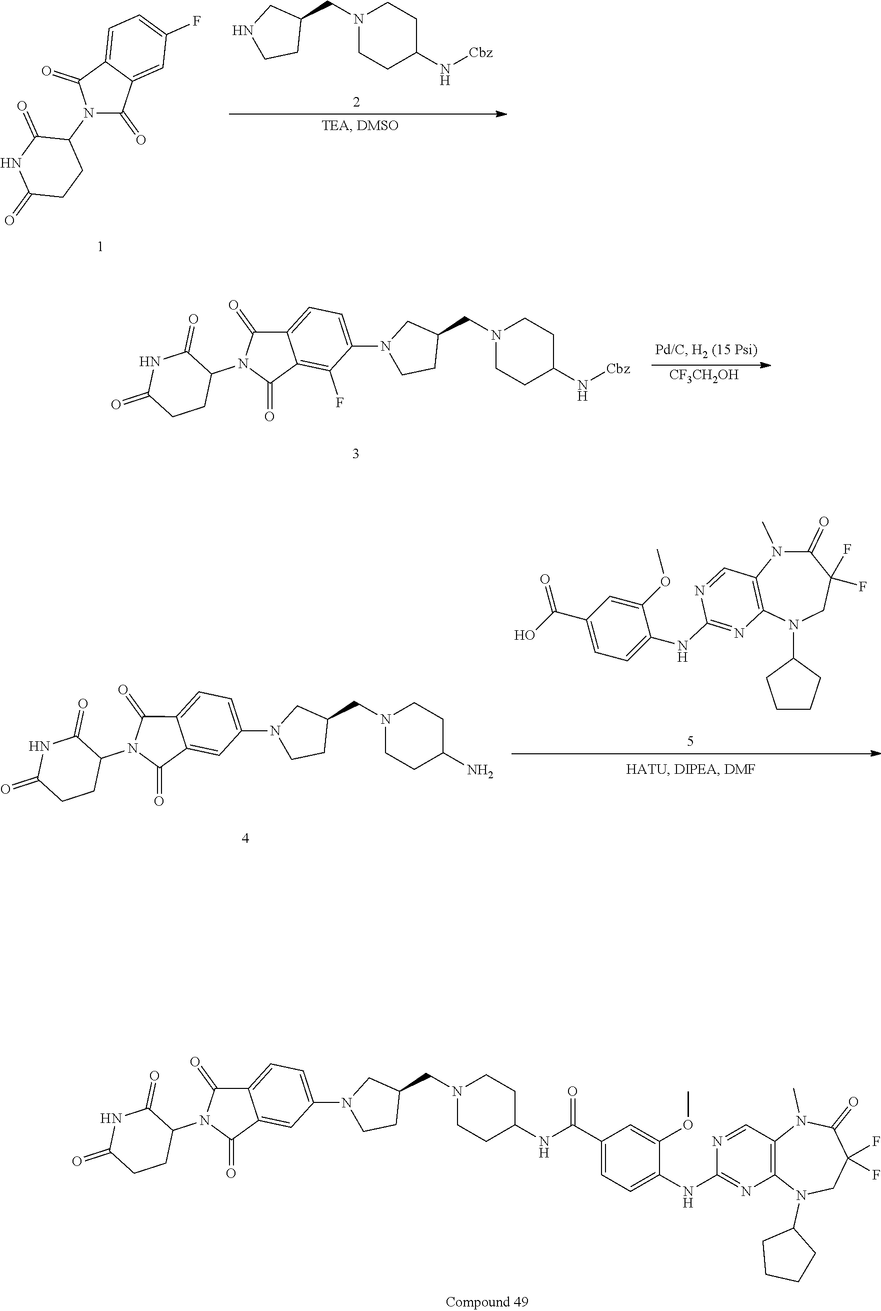

The compound 49 was synthesized by the method described in the scheme similar to the method described in Example 44.

MS(M+H)⁺=869.7, ¹H NMR (400 MHz, DMSO-d₆) δ=11.06 (s, 1H), 8.33-8.23 (m, 2H), 8.10 (d, J=7.6 Hz, 1H), 7.96 (s, 1H), 7.65 (d, J=8.4 Hz, 1H), 7.54-7.43 (m, 2H), 6.89 (d, J=2.0 Hz, 1H), 6.80 (dd, J=2.0, 8.6 Hz, 1H), 5.05 (dd, J=5.4, 12.8 Hz, 1H), 4.78-4.74 (m, 1H), 4.05 (t, J=14.1 Hz, 2H), 3.94 (s, 3H), 3.86-3.74 (m, 1H), 3.59-3.46 (m, 2H), 3.46-3.37 (m, 1H), 3.30 (s, 3H), 3.18-3.13 (m, 1H), 3.02-2.82 (m, 3H), 2.68-2.52 (m, 4H), 2.40-2.33 (m, 2H), 2.15-1.90 (m, 6H), 1.80-1.57 (m, 10H).

### Example 50. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1-(2-(1-(2-(2,6-dioxopiperidin-3-yl)-1,3-dioxoisoindolin-4-yl)pyrrolidin-3-yl)ethyl)piperidin-4-yl)methyl)-3-methoxybenzamide (Compound 50)

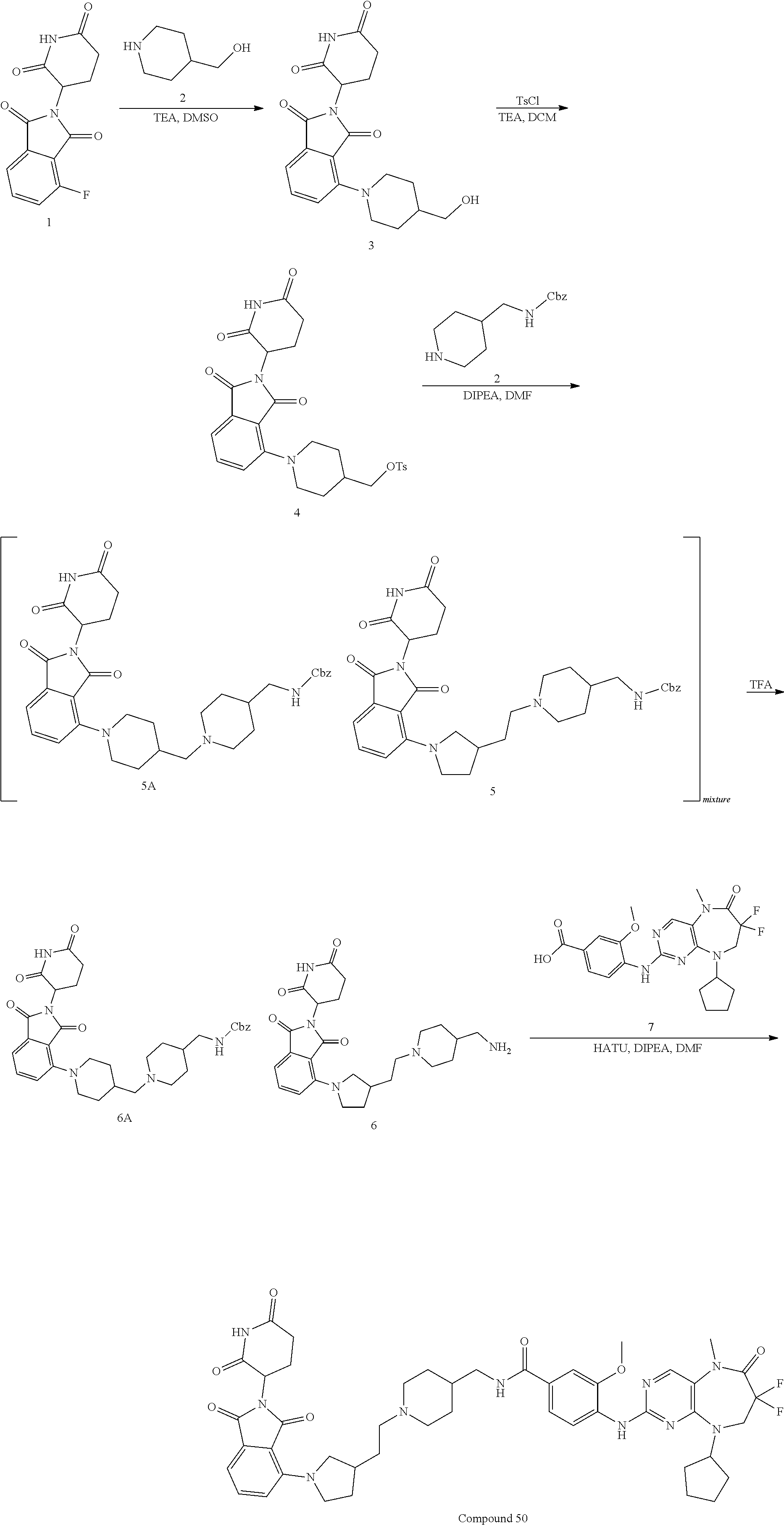

### Step 1. Synthesis of 2-(2,6-dioxopiperidin-3-yl)-4-(4-(hydroxymethyl)piperidin-1-yl)isoindoline-1,3-dione (3)

To a solution of 2-(2,6-dioxopiperidin-3-yl)-4-fluoroisoindoline-1,3-dione (2 g, 7.24 mmol) in DMSO (20 mL) were added TEA (3.66 g, 36.20 mmol, 5.04 mL) and piperidin-4-ylmethanol (833.92 mg, 7.24 mmol). The mixture was stirred at 100° C. for 16 hr. LCMS showed a main peak with desired mass. The reaction mixture was diluted with brine (60 mL), extracted with EtOAc (40 mL×3). The combined organic layers were washed with brine (100 mL×2), dried over Na₂SO₄, filtered. The filtrate was concentrated under reduced pressure to afford 2-(2,6-dioxopiperidin-3-yl)-4-(4-(hydroxymethyl)piperidin-1-yl)isoindoline-1,3-dione (2.7 g, crude) as a yellow oil. MS(M+H)⁺=371.9

### Step 2. Synthesis of (1-(2-(2,6-dioxopiperidin-3-yl)-1,3-dioxoisoindolin-4-yl)piperidin-4-yl)methyl 4-methylbenzenesulfonate (4)

To a solution of 2-(2,6-dioxopiperidin-3-yl)-4-(4-(hydroxymethyl)piperidin-1-yl)isoindoline-1,3-dione (2.7 g, crude) and TosCl (2.08 g, 10.91 mmol) in DCM (80 mL) was added TEA (2.21 g, 21.81 mmol, 3.04 mL). The solution was stirred at 25° C. for 16 hr. LCMS showed a main peak with desired mass. The mixture was concentrated under reduced pressure to give the residue. The residue was purified by flash silica gel chromatography (25 g SepaFlash® Silica Flash Column, Eluent of 0˜100% EtOAc/Petroleum ether gradient @100 mL/min) to afford (1-(2-(2,6-dioxopiperidin-3-yl)-1,3-dioxoisoindolin-4-yl)piperidin-4-yl)methyl 4-methylbenzenesulfonate (1.7 g, 3.01 mmol, 41.38% yield, 93% purity) as a yellow solid. MS(M+H)⁺=525.9

### Step 3. Synthesis of a mixture of benzyl ((1-((1-(2-(2,6-dioxopiperidin-3-yl)-1,3-dioxoisoindolin-4-yl)piperidin-4-yl)methyl) piperidin-4-yl)methyl)carbamate (5A) and benzyl ((1-(2-(1-(2-(2,6-dioxopiperidin-3-yl)-1,3-dioxoisoindolin-4-yl)pyrrolidin-3-yl)ethyl)piperidin-4-yl)methyl)carbamate (5)

To a solution of (1-(2-(2,6-dioxopiperidin-3-yl)-1,3-dioxoisoindolin-4-yl)piperidin-4-yl)methyl 4-methylbenzenesulfonate (200 mg, 380.54 μmol) in DMF (4 mL) were added benzyl (piperidin-4-ylmethyl)carbamate (113.39 mg, 456.64 μmol), DIPEA (147.55 mg, 1.14 mmol, 198.85 μL), the mixture was stirred at 60° C. for 16 h. LCMS showed a main peak with desired mass. The reaction mixture was diluted with brine (10 mL), extracted with EtOAc (10 mL×3). The combined organic layers were washed with brine (20 mL×2), dried over Na₂SO₄, filtered. The filtrate was concentrated under reduced pressure to give a residue. The residue was purified by flash silica gel chromatography (Biotage; 10 g SepaFlash® Silica Flash Column, Eluent of 20˜100% EtOAc/Petroleum ether to 10% Methanol/EtOAc gradient @40 mL/min) to afford a mixture of benzyl ((1-((1-(2-(2,6-dioxopiperidin-3-yl)-1,3-dioxoisoindolin-4-yl)piperidin-4-yl)methyl)piperidin-4-yl)methyl)carbamate and benzyl ((1-(2-(1-(2-(2,6-dioxopiperidin-3-yl)-1,3-dioxoisoindolin-4-yl)pyrrolidin-3-yl)ethyl)piperidin-4-yl)methyl)carbamate (160 mg, 265.92 μmol, 69.88% yield, 100% purity) as a yellow solid. MS(M+H)⁺=602.1

### Step 4. Synthesis of 4-(4-((4-(aminomethyl)piperidin-1-yl)methyl)piperidin-1-yl)-2-(2,6-dioxopiperidin-3-yl)isoindoline-1,3-dione (6A) and 4-(3-(2-(4-(aminomethyl)piperidin-1-yl)ethyl)pyrrolidin-1-yl)-2-(2,6-dioxopiperidin-3-yl)isoindoline-1,3-dione (6)

A mixture of benzyl ((1-((1-(2-(2,6-dioxopiperidin-3-yl)-1,3-dioxoisoindolin-4-yl)piperidin-4-yl)methyl)piperidin-4-yl)methyl)carbamate and benzyl ((1-(2-(1-(2-(2,6-dioxopiperidin-3-yl)-1,3-dioxoisoindolin-4-yl)pyrrolidin-3-yl)ethyl)piperidin-4-yl)methyl)carbamate (160 mg, 265.92 μmol) in TFA (2 mL) was stirred at 60° C. for 16 h. LCMS showed a main peak with desired mass. The mixture was concentrated under reduced pressure. The residue was purified by reversed-phase HPLC (column: Phenomenex Synergi Polar-RP 100*25 mm*4 μm; mobile phase: [water (TFA)-ACN]; B %: 14%-34%, 7 min). The eluent was lyophilized to afford 4-(4-((4-(aminomethyl)piperidin-1-yl)methyl)piperidin-1-yl)-2-(2,6-dioxopiperidin-3-yl)isoindoline-1,3-dione (20 mg, 29.51 μmol, 11.10% yield, 69% purity) and 4-(3-(2-(4-(aminomethyl)piperidin-1-yl)ethyl)pyrrolidin-1-yl)-2-(2,6-dioxopiperidin-3-yl)isoindoline-1,3-dione (70 mg, 146.72 μmol, 55.18% yield, 98% purity) as a yellow solid. MS(M+H)⁺=468.0

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1-(2-(1-(2-(2,6-dioxopiperidin-3-yl)-1,3-dioxoisoindolin-4-yl)pyrrolidin-3-yl)ethyl)piperidin-4-yl)methyl)-3-methoxybenzamide (Compound 50)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (66.99 mg, 149.71 μmol) in DMF (2 mL) were added HATU (85.39 mg, 224.57 μmol) and DIPEA (58.05 mg, 449.14 μmol, 78.23 μL), the mixture was stirred at 20° C. for 0.5 h, 4-(3-(2-(4-(aminomethyl)piperidin-1-yl)ethyl)pyrrolidin-1-yl)-2-(2,6-dioxopiperidin-3-yl)isoindoline-1,3-dione (70 mg, 149.71 μmol) was added, the mixture was stirred at 20° C. for 1 h. LCMS showed a main peak with desired mass. The reaction mixture was diluted with brine (10 mL), extracted with EtOAc (10 mL×3). The combined organic layers were washed with brine (20 mL×2), dried over Na₂SO₄, filtered. The filtrate was concentrated under reduced pressure to give a residue. The residue was purified by prep-TLC (SiO₂, DCM:MeOH=10:1) and re-purified by reversed-phase HPLC (column: Waters Xbridge 150*25 mm*5 μm; mobile phase: [water(NH₄HCO₃)-ACN]; B %: 40%-70%, 9 min). The eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-((1-(2-(1-(2-(2,6-dioxopiperidin-3-yl)-1,3-dioxoisoindolin-4-yl)pyrrolidin-3-yl)ethyl)piperidin-4-yl)methyl)-3-methoxybenzamide (50.2 mg, 54.29 μmol, 36.26% yield, 97% purity) as a yellow solid. MS(M+H)⁺=897.0

¹H NMR (400 MHz, DMSO-d₆) δ=11.05 (s, 1H), 8.39-8.34 (m, 1H), 8.28-8.23 (m, 2H), 7.96 (s, 1H), 7.58-7.46 (m, 3H), 7.13-7.06 (m, 2H), 5.11-5.02 (m, 1H), 4.82-4.70 (m, 1H), 4.04 (t, J=13.9 Hz, 2H), 3.93 (s, 3H), 3.68-3.55 (m, 2H), 3.52-3.42 (m, 1H), 3.31-3.27 (m, 5H), 3.18-3.11 (m, 2H), 2.95-2.80 (m, 3H), 2.62-2.54 (m, 2H), 2.33-2.29 (m, 2H), 2.25-2.18 (m, 1H), 2.13-2.06 (m, 1H), 2.04-1.92 (m, 3H), 1.87-1.77 (m, 2H), 1.74-1.68 (m, 2H), 1.67-1.62 (m, 3H), 1.61-1.54 (m, 6H), 1.23-1.12 (m, 2H).

Ir[dF(CF₃)ppy]₂ (dtbpy)(PF₆) (33.28 mg, 29.66 μmol), NiCl₂

### Example 51. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(1-(2-(2,6-dioxopiperidin-3-yl)-1,3-dioxoisoindolin-4-yl)pyrrolidin-3-yl)piperazin-1-yl)-3-methoxybenzamide (Compound 51)

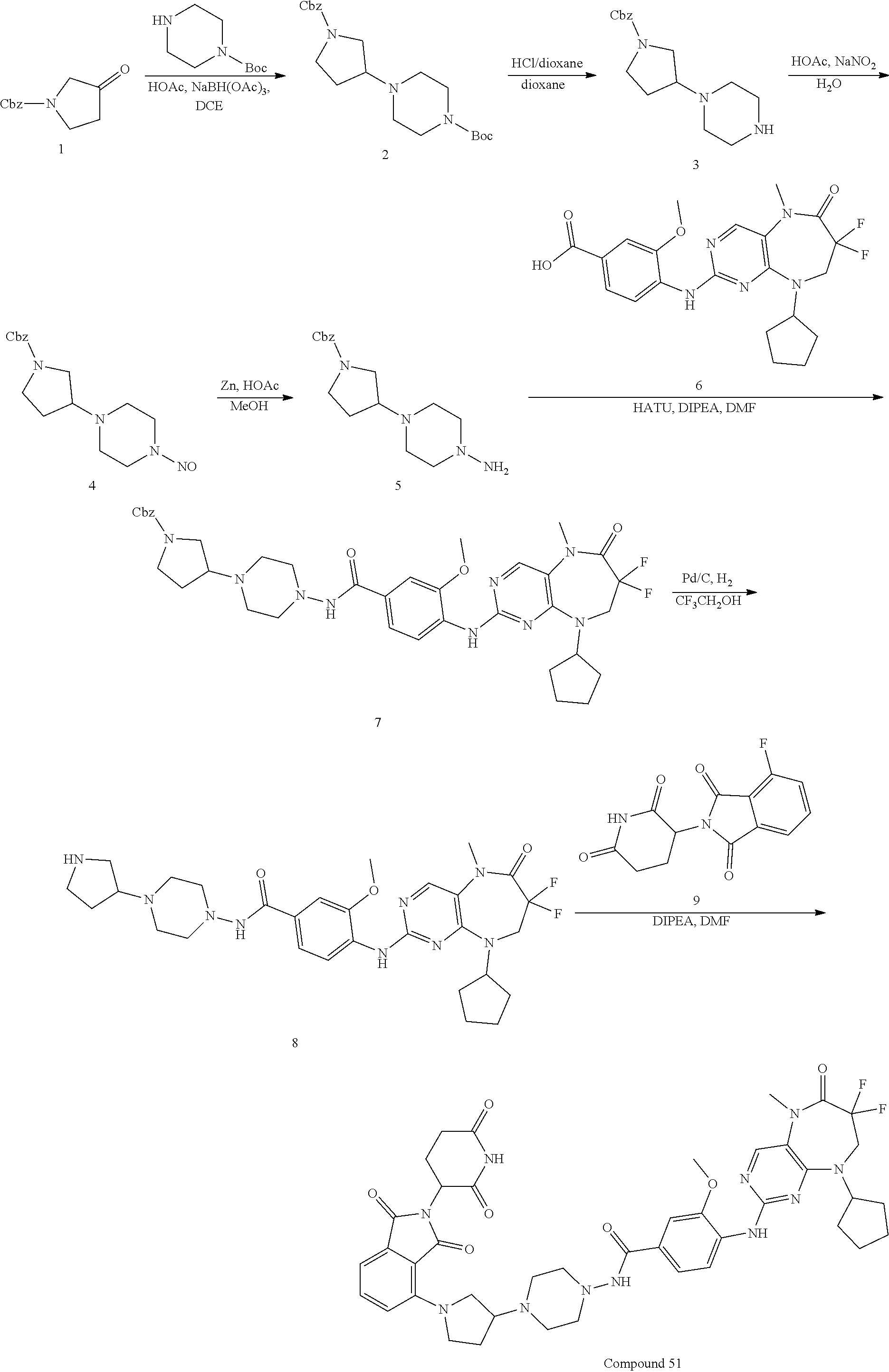

### Step 1. Synthesis of tert-butyl 4-(1-((benzyloxy)carbonyl)pyrrolidin-3-yl)piperazine-1-carboxylate (2)

To a solution of benzyl 3-oxopyrrolidine-1-carboxylate (1 g, 4.56 mmol) in DCE (10 mL) were added HOAc (328.70 mg, 5.47 mmol, 313.05 μL) and tert-butyl piperazine-1-carboxylate (934.50 mg, 5.02 mmol). Then NaBH(OAc)₃ (1.93 g, 9.12 mmol) was added at 0° C. and the resulting mixture was stirred at 25° C. for 12 h. LCMS showed a peak (˜90%) with desired mass. Saturated sodium bicarbonate solution (150 mL) was added to the mixture slowly at 0° C. Then the mixture was extracted with DCM (50 mL×3). The combined organic layers were washed with saturated brine (50 mL), dried over anhydrous sodium sulfate, filtered and concentrated under reduced pressure. The residue was purified by flash silica gel chromatography (ISCO®; 20 g SepaFlash® Silica Flash Column, Eluent of 0˜20% petroleum ether:EtOAc/EtOH (v/v=5/1) gradient @60 mL/min) to afford tert-butyl 4-(1-((benzyloxy)carbonyl)pyrrolidin-3-yl)piperazine-1-carboxylate (1.75 g, 4.43 mmol, 97.22% yield, 98.7% purity) as light yellow oil. MS(M+H)⁺=390.3

### Step 2. Synthesis of benzyl 3-(piperazin-1-yl)pyrrolidine-1-carboxylate (3)

To a solution of tert-butyl 4-(1-((benzyloxy)carbonyl)pyrrolidin-3-yl)piperazine-1-carboxylate (1.75 g, 4.49 mmol) in dioxane (5 mL) was added HCl/dioxane (4 M, 10 mL) at 25° C. The mixture was stirred at 25° C. for 2 h. TLC (Petroleum ether:EtOAc=1:2) indicated one new spot formed. The mixture was concentrated under reduced pressure to afford benzyl 3-(piperazin-1-yl)pyrrolidine-1-carboxylate (2 g, 3.89 mmol, 86.48% yield, 63.3% purity, HCl) as a white solid. MS(M+H)⁺=290.2

### Step 3. Synthesis of benzyl 3-(4-nitrosopiperazin-1-yl)pyrrolidine-1-carboxylate (4)

To a solution of benzyl 3-(piperazin-1-yl)pyrrolidine-1-carboxylate (2 g, 6.14 mmol, HCl) in H₂O (5 mL) were added a solution of NaNO₂ (635.25 mg, 9.21 mmol) in H₂O (5 mL) and HOAc (552.91 mg, 9.21 mmol, 526.58 μL) at 0° C. The mixture was stirred at 25° C. for 4 h under N₂. LCMS showed ˜68% of desired mass was detected. The reaction mixture was diluted with water (50 mL) and extracted with EtOAc (50 mL×3). The combined organic layers were washed with saturated brine (50 mL), dried over anhydrous sodium sulfate, filtered and concentrated under reduced pressure to give benzyl 3-(4-nitrosopiperazin-1-yl)pyrrolidine-1-carboxylate (2 g, crude) as yellow oil. MS(M+H)⁺=319.2

### Step 4. Synthesis of benzyl 3-(4-aminopiperazin-1-yl)pyrrolidine-1-carboxylate (5)

To a solution of benzyl 3-(4-nitrosopiperazin-1-yl)pyrrolidine-1-carboxylate (2 g, crude) in MeOH (10 mL) was added Zn (1.77 g, 27.07 mmol) at 0° C. Then HOAc (754.49 mg, 12.56 mmol, 718.56 μL) was added slowly to the mixture at 0° C. The mixture was stirred at 25° C. for 2 h. TLC (petroleum ether:EtOAc=1:2) indicated one new spot formed. The mixture was filtered to remove insoluble solid. The filtrate was concentrated under reduced pressure to give a residue. The residue was purified by reverse flash column (column: 330 g Flash Column Welch Ultimate XB_C18 20-40 μm; 120 A; mobile phase: [water(NH₃·H₂O)-ACN]; B %: 0%-30%, 20 min) followed by lyophilization to afford benzyl 3-(4-aminopiperazin-1-yl)pyrrolidine-1-carboxylate (0.46 g, 1.27 mmol, 20.18% yield, 83.9% purity) as light yellow oil. MS(M+H)⁺=305.1

### Step 5. Synthesis of benzyl 3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperazin-1-yl)pyrrolidine-1-carboxylate (7)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (293.99 mg, 657.06 μmol) in DMF (5 mL) were added DIPEA (254.76 mg, 1.97 mmol, 343.34 μL) and HATU (499.67 mg, 1.31 mmol). Then benzyl 3-(4-aminopiperazin-1-yl)pyrrolidine-1-carboxylate (0.2 g, 657.06 μmol) was added to the mixture after 0.5 h. The mixture was stirred at 25° C. for 2 h. LCMS showed ˜45% of desired mass was detected. The reaction mixture was diluted with water (50 mL) and extracted with EtOAc (50 mL×3). The combined organic layers were washed with saturated brine (50 mL), dried over anhydrous sodium sulfate, filtered and concentrated under reduced pressure to give a residue. The residue was purified by flash silica gel chromatography (ISCO®; 20 g Sepa Flash® Silica Flash Column, Eluent of 0˜70% petroleum ether:EtOAc/ethanol (1:1) gradient @60 mL/min) to afford benzyl 3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperazin-1-yl)pyrrolidine-1-carboxylate (470 mg, 620.64 μmol, 94.46% yield, 96.9% purity) as brown oil. MS(M+H)⁺=734.3

### Step 6. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(4-(pyrrolidin-3-yl)piperazin-1-yl)benzamide (8)

To a mixture of Pd/C (0.1 g, 64.05 μmol, 10% purity) in CF₃CH₂OH (10 mL) was added benzyl 3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperazin-1-yl)pyrrolidine-1-carboxylate (0.47 g, 640.50 μmol). The mixture was stirred at 25° C. for 12 h under H₂ (15 psi). LCMS showed ˜90% of desired mass was detected. The mixture was filtered to remove the catalyst. The filtrate was concentrated under reduced pressure to give 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(4-(pyrrolidin-3-yl)piperazin-1-yl)benzamide (320 mg, crude) as yellow oil. MS(M+H)⁺=600.3

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(1-(2-(2,6-dioxopiperidin-3-yl)-1,3-dioxoisoindolin-4-yl)pyrrolidin-3-yl)piperazin-1-yl)-3-methoxybenzamide (Compound 51)

To a solution of 2-(2,6-dioxopiperidin-3-yl)-4-fluoroisoindoline-1,3-dione (46.06 mg, 166.76 μmol) in DMF (3 mL) were added DIPEA (43.10 mg, 333.51 μmol, 58.09 μL) and 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(4-(pyrrolidin-3-yl)piperazin-1-yl)benzamide (100 mg, 166.76 μmol) at 25° C. The mixture was stirred at 60° C. for 2 h. LCMS showed ˜30% of desired mass was detected. The reaction mixture was diluted with water (20 mL) and extracted with EtOAc (30 mL×3). The combined organic layers were washed with saturated brine (50 mL), dried over anhydrous sodium sulfate, filtered and concentrated under reduced pressure to give a residue. The residue was purified by prep-HPLC (column: Phenomenex luna C18 150×25 mm×10 μm; mobile phase: [water(FA)-ACN]; B %: 14%-44%, 10 min) and then by prep-HPLC (column: Waters Xbridge 150×25 mm×5 μm; mobile phase: [water(NH₄HCO₃)-ACN]; B %: 38%-68%, 8 min) followed by lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(1-(2-(2,6-dioxopiperidin-3-yl)-1,3-dioxoisoindolin-4-yl)pyrrolidin-3-yl)piperazin-1-yl)-3-methoxybenzamide (15 mg, 17.21 μmol, 10.32% yield, 98.2% purity) as a yellow solid. MS(M+H)⁺=856.2

¹H NMR (400 MHz, DMSO-d₆) δ=11.06 (s, 1H), 9.37 (s, 1H), 8.32-8.23 (m, 2H), 7.97 (s, 1H), 7.57 (t, J=7.6 Hz, 1H), 7.46-7.39 (m, 2H), 7.17-7.09 (m, 2H), 5.08 (dd, J=5.3, 12.5 Hz, 1H), 4.82-4.72 (m, 1H), 4.04 (t, J=14.1 Hz, 2H), 3.93 (s, 3H), 3.71-3.51 (m, 4H), 3.32-3.31 (m, 3H), 3.01-2.83 (m, 6H), 2.67-2.56 (m, 6H), 2.25-2.16 (m, 1H), 2.04-1.91 (m, 3H), 1.85-1.69 (m, 3H), 1.62-1.58 (m, 4H).

### Example 52. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(1-(2-(2,6-dioxopiperidin-3-yl)-1,3-dioxoisoindolin-4-yl)pyrrolidin-3-yl)piperazin-1-yl)-3-methoxybenzamide (Compound 52)

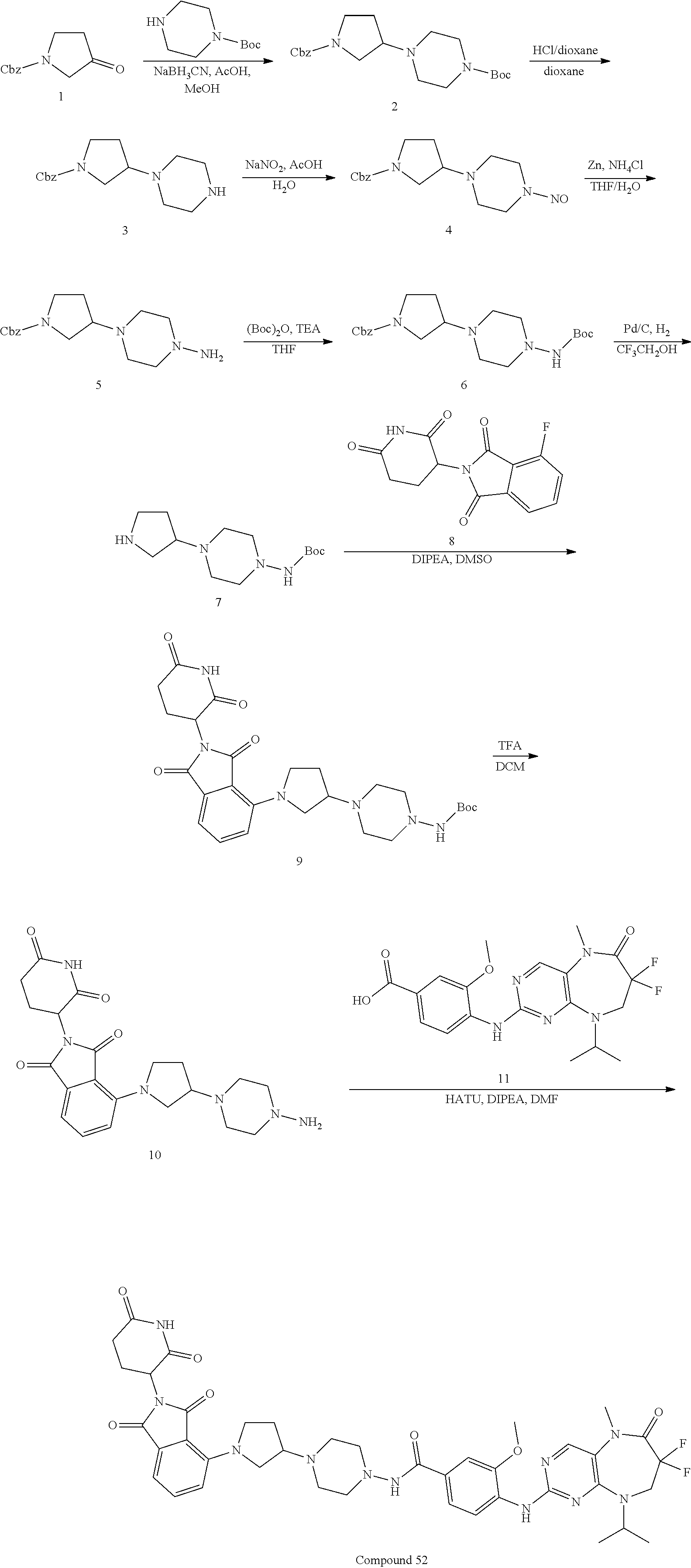

### Step 1. Synthesis of tert-butyl 4-(1-((benzyloxy)carbonyl)pyrrolidin-3-yl)piperazine-1-carboxylate (2)

To a solution of benzyl 3-oxopyrrolidine-1-carboxylate (9 g, 41.05 mmol) and tert-butyl piperazine-1-carboxylate (7.65 g, 41.05 mmol) in MeOH (100 mL) was added AcOH (2.47 g, 41.05 mmol, 2.35 mL) at 0° C. Then was slowly added NaBH₃CN (7.74 g, 123.15 mmol) at 0° C. and the resulting mixture was stirred at 20° C. for 16 h. LCMS showed starting material was consumed completely and 31% peak with desired mass was detected. The reaction mixture was concentrated in vacuum. The residue was purified by flash silica gel chromatography (80 g SepaFlash® Silica Flash Column, Eluent of 0˜80% EtOAc/Petroleum ether gradient @100 mL/min) to afford tert-butyl 4-(1-((benzyloxy)carbonyl)pyrrolidin-3-yl)piperazine-1-carboxylate (8.9 g, 22.85 mmol, 55.66% yield) as a white solid. MS(M+H)⁺=390.2

### Step 2. Synthesis of benzyl 3-(piperazin-1-yl)pyrrolidine-1-carboxylate (3)

To a solution of tert-butyl 4-(1-((benzyloxy)carbonyl)pyrrolidin-3-yl)piperazine-1-carboxylate (8.9 g, 22.85 mmol) in dioxane (10 mL) was added HCl/dioxane (4 M, 40 mL) at 20° C. and the resulting mixture was stirred at 20° C. for 1 h. LCMS showed starting material was consumed completely and a peak with desired mass. The reaction mixture was concentrated in vacuum to afford benzyl 3-(piperazin-1-yl)pyrrolidine-1-carboxylate (7.5 g, crude, HCl salt) as a white solid. MS(M+H)⁺=290.2

### Step 3. Synthesis of benzyl 3-(4-nitrosopiperazin-1-yl)pyrrolidine-1-carboxylate (4)

To a solution of benzyl 3-(piperazin-1-yl)pyrrolidine-1-carboxylate (7.5 g, 23.02 mmol, HCl salt) in H₂O (100 mL) was added NaNO₂ (4.76 g, 69.05 mmol) at 0° C., then AcOH (5.53 g, 92.07 mmol, 5.27 mL) was added drop-wise at 0° C. and the resulting mixture was stirred at 20° C. for 12 h. LCMS showed the starting material was consumed completely and a peak (82%) with desired mass. The reaction mixture at 0° C. was adjusted the pH=10 by adding saturated-NaHCO₃, the resulting mixture was extracted with EtOAc (150 mL×3). The organic layer was dried over Na₂SO₄, filtered and concentrated to afford benzyl 3-(4-nitrosopiperazin-1-yl)pyrrolidine-1-carboxylate (4.74 g, 14.89 mmol, 64.68% yield) as a yellow oil. MS(M+H)⁺=319.4

### Step 4. Synthesis of benzyl 3-(4-aminopiperazin-1-yl)pyrrolidine-1-carboxylate (5)

To a solution of benzyl 3-(4-nitrosopiperazin-1-yl)pyrrolidine-1-carboxylate (4.7 g, 14.76 mmol) in THF (60 mL) and H₂O (20 mL) was added NH₄Cl (3.16 g, 59.05 mmol) at 0° C., then Zn (3.86 g, 59.05 mmol) was added portion wise at 0° C. and the resulting mixture was stirred at 20° C. for 12 h. LCMS showed the starting material was consumed completely and a peak with desired mass. The reaction mixture was diluted with THF (200 mL) and filtered. The filtrate was concentrated in vacuum to afford benzyl 3-(4-aminopiperazin-1-yl)pyrrolidine-1-carboxylate (9 g, crude) as a white solid. MS(M+H)⁺=305.1

### Step 5. Synthesis of benzyl 3-(4-((tert-butoxycarbonyl)amino)piperazin-1-yl)pyrrolidine-1-carboxylate (6)

To a solution of benzyl 3-(4-aminopiperazin-1-yl)pyrrolidine-1-carboxylate (9 g, 29.57 mmol) in THF (100 mL) were added TEA (8.98 g, 88.70 mmol, 12.35 mL) and (Boc)₂O (6.45 g, 29.57 mmol, 6.79 mL) at 20° C. and the resulting mixture was stirred at 20° C. for 12 h. LCMS showed the starting material was consumed completely and a peak (79%) with desired mass. The reaction mixture was diluted with H₂O (200 mL) and extracted with EtOAc (200 mL×3). The organic layer was dried over Na₂SO₄, filtered and concentrated. The residue was purified by flash silica gel chromatography (50 g SepaFlash® Silica Flash Column, Eluent of 0˜100% EtOAc/Petroleum ether to 0˜10% Dichloromethane/Methanol gradient @100 mL/min) and re-purified by flash silica gel chromatography (50 g SepaFlash® Silica Flash Column, Eluent of 0˜100% EtOAc/Petroleum ether gradient @100 mL/min) to afford benzyl 3-(4-((tert-butoxycarbonyl)amino)piperazin-1-yl)pyrrolidine-1-carboxylate (1.2 g, 2.82 mmol, 9.53% yield, 95% purity) as a Colorless oil. MS(M+H)⁺=405.2

### Step 6. Synthesis of tert-butyl (4-(pyrrolidin-3-yl)piperazin-1-yl)carbamate (7)

To a solution of benzyl 3-(4-((tert-butoxycarbonyl)amino)piperazin-1-yl)pyrrolidine-1-carboxylate (600 mg, 1.48 mmol) in CF₃CH₂OH (12 mL) was added Pd/C (0.2 g, 10% purity) under N₂ atmosphere. The suspension was degassed and purged with H₂ for 3 times. The mixture was stirred at 20° C. for 12 h under H₂ (15 Psi). LCMS showed 42% of starting material remained and a peak with desired mass. The reaction mixture was stirred at 40° C. for 12 h. LCMS showed starting material was consumed completely. The reaction mixture was diluted with CF₃CH₂OH (15 mL) and filtered. The filtrate was concentrated in vacuum to tert-butyl (4-(pyrrolidin-3-yl)piperazin-1-yl)carbamate (404 mg, crude) as a yellow oil. MS(M+H)⁺=271.1

### Step 7. Synthesis of tert-butyl (4-(1-(2-(2,6-dioxopiperidin-3-yl)-1,3-dioxoisoindolin-4-yl)pyrrolidin-3-yl)piperazin-1-yl)carbamate (9)

To a solution of 2-(2,6-dioxopiperidin-3-yl)-4-fluoroisoindoline-1,3-dione (200 mg, 724.06 μmol) in DMSO (4 mL) were added DIPEA (280.74 mg, 2.17 mmol, 378.36 μL) and tert-butyl (4-(pyrrolidin-3-yl)piperazin-1-yl)carbamate (195.76 mg, 724.06 μmol) at 20° C. and the resulting mixture was stirred at 100° C. for 12 h. LCMS showed the starting material was consumed completely and a peak (46%) with desired mass. The reaction mixture was diluted with H₂O (12 mL) and extracted with EtOAc (12 mL×3). The organic layer was washed with brine (12 mL×3), dried over Na₂SO₄, filtered and concentrated. The residue was purified by flash silica gel chromatography (10 g SepaFlash® Silica Flash Column, Eluent of 0˜100% EtOAc/Petroleum ether gradient @100 mL/min) to afford tert-butyl (4-(1-(2-(2,6-dioxopiperidin-3-yl)-1,3-dioxoisoindolin-4-yl)pyrrolidin-3-yl)piperazin-1-yl)carbamate (134 mg, 249.38 μmol, 34.44% yield, 98% purity) as a yellow oil. MS(M+H)⁺=527.3

### Step 8. Synthesis of 4-(3-(4-aminopiperazin-1-yl)pyrrolidin-1-yl)-2-(2,6-dioxopiperidin-3-yl)isoindoline-1,3-dione (10)

To a solution of tert-butyl (4-(1-(2-(2,6-dioxopiperidin-3-yl)-1,3-dioxoisoindolin-4-yl)pyrrolidin-3-yl)piperazin-1-yl)carbamate (134 mg, 254.47 μmol) in DCM (2 mL) was added TFA (145.08 mg, 1.27 mmol, 94.21 μL) at 20° C. and the resulting mixture was stirred at 20° C. for 1 h. LCMS showed starting material was consumed completely and a peak (90%) with desired mass. The reaction mixture was concentrated in vacuum to afford 4-(3-(4-aminopiperazin-1-yl)pyrrolidin-1-yl)-2-(2,6-dioxopiperidin-3-yl)isoindoline-1,3-dione (138 mg, crude, TFA) as a yellow oil. MS(M+H)⁺=427.3

### Step 9. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(1-(2-(2,6-dioxopiperidin-3-yl)-1,3-dioxoisoindolin-4-yl)pyrrolidin-3-yl)piperazin-1-yl)-3-methoxybenzamide (Compound 52)

To a solution of 4-((7,7-difluoro-9-isopropyl-5-methyl-6-oxo-6,7,8,9-tetrahydro-5H-pyrimido[4,5-b][1,4]diazepin-2-yl)amino)-3-methoxybenzoic acid (80 mg, 189.84 μmol) in DMF (2 mL) were added HATU (79.40 mg, 208.83 μmol) and DIPEA (49.07 mg, 379.69 μmol, 66.14 μL). The mixture was stirred at 20° C. for 10 min and a solution of 4-(3-(4-aminopiperazin-1-yl)pyrrolidin-1-yl)-2-(2,6-dioxopiperidin-3-yl)isoindoline-1,3-dione (123.13 mg, 227.81 μmol, TFA) in DMF (2 mL) with DIPEA (98.14 mg, 759.38 μmol, 132.27 μL) was added and the resulting mixture was stirred at 20° C. for 1 h. LCMS showed all starting material was consumed completely and a peak (70%) with desired mass. The reaction mixture was diluted with H₂O (12 mL) and extracted with EtOAc (12 mL×3). The organic layer was dried over Na₂SO₄, filtered and concentrated. The residue was purified by prep-HPLC (column: Phenomenex Synergi Polar-RP 100*25 mm*4 μm; mobile phase: [water(TFA)-ACN]; B %: 27%-47%, 7 min) and the eluent was lyophilized to afford 4-((7,7-difluoro-9-isopropyl-5-methyl-6-oxo-6,7,8,9-tetrahydro-5H-pyrimido[4,5-b][1,4]diazepin-2-yl)amino)-N-(4-(1-(2-(2,6-dioxopiperidin-3-yl)-1,3-dioxoisoindolin-4-yl) pyrrolidin-3-yl)piperazin-1-yl)-3-methoxybenzamide (62.3 mg, 61.38 μmol, 32.33% yield, 93% purity, TFA salt) as a yellow solid. MS(M+H)⁺=830.4

¹H NMR (400 MHz, CD₃CN) δ=9.64 (br s, 1H), 8.97 (br d, J=7.0 Hz, 1H), 8.31 (br s, 1H), 8.05 (d, J=8.4 Hz, 1H), 7.92 (s, 1H), 7.62-7.54 (m, 1H), 7.47-7.36 (m, 2H), 7.23 (d, J=7.0 Hz, 1H), 7.12-7.04 (m, 1H), 5.03-4.90 (m, 2H), 4.29-4.13 (m, 2H), 4.01 (br t, J=12.2 Hz, 2H), 3.95-3.78 (m, 6H), 3.76-3.62 (m, 3H), 3.57-3.47 (m, 1H), 3.31 (s, 7H), 2.85-2.60 (m, 3H), 2.52-2.32 (m, 2H), 2.15-2.06 (m, 1H), 1.23 (d, J=6.7 Hz, 6H).

### Example 53. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1-((1-(2-(2,6-dioxopiperidin-3-yl)-1,3-dioxoisoindolin-4-yl)piperidin-4-yl)methyl)piperidin-4-yl)methyl)-3-methoxybenzamide (Compound 53)

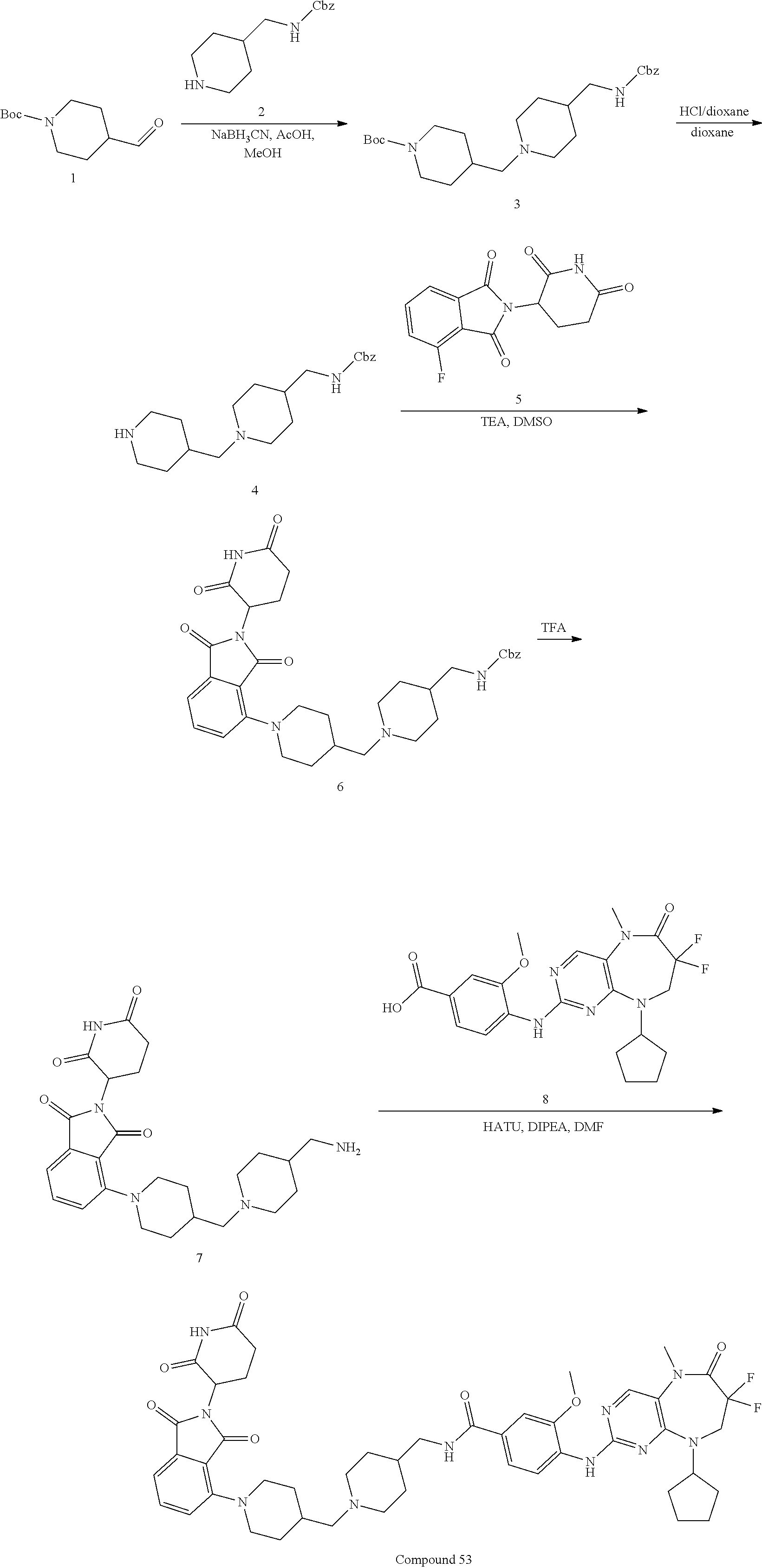

### Step 1. Synthesis of tert-butyl 4-((4-((((benzyloxy)carbonyl)amino)methyl)piperidin-1-yl)methyl)piperidine-1-carboxylate (3)

To a solution of tert-butyl 4-formylpiperidine-1-carboxylate (500 mg, 2.34 mmol) in MeOH (10 mL) were added benzyl (piperidin-4-ylmethyl)carbamate (640.38 mg, 2.58 mmol) and AcOH (14.08 mg, 234.44 μmol, 13.41 μL), the mixture was stirred at 20° C. for 0.5 h, then NaBH₃CN (220.99 mg, 3.52 mmol) was added and the resulting mixture was stirred at 20° C. for 16 h. LCMS showed a main peak with desired mass. The reaction mixture was diluted with H₂O (20 mL), extracted with EtOAc (3×20 mL). The combined organic layers were washed with brine (20 mL×2), dried over Na₂SO₄, filtered. The filtrate was concentrated under reduced pressure. The residue was purified by flash silica gel chromatography (Biotage; 20 g SepaFlash® Silica Flash Column, Eluent of 20˜70% EtOAc/Petroleum ether @40 mL/min) to afford tert-butyl 4-((4-((((benzyloxy)carbonyl)amino)methyl)piperidin-1-yl)methyl)piperidine-1-carboxylate (800 mg, 1.47 mmol, 62.80% yield, 82% purity) as colorless oil. MS(M+H)⁺=446.1

### Step 2. Synthesis of benzyl ((1-(piperidin-4-ylmethyl)piperidin-4-yl)methyl)carbamate (4)

To a solution of tert-butyl 4-((4-((((benzyloxy)carbonyl)amino)methyl)piperidin-1-yl)methyl)piperidine-1-carboxylate (200 mg, 448.84 μmol) in dioxane (2 mL) was added HCl/dioxane (4 M, 2 mL, 17.82 eq), the mixture was stirred at 20° C. for 1 h. LCMS showed a main peak with desired mass. The mixture was concentrated under reduced pressure to afford benzyl ((1-(piperidin-4-ylmethyl)piperidin-4-yl)methyl)carbamate (150 mg, crude, HCl salt) as a white solid, which was used into the next step directly. MS(M+H)⁺=346.0

### Step 3. Synthesis of benzyl ((1-((1-(2-(2,6-dioxopiperidin-3-yl)-1,3-dioxoisoindolin-4-yl)piperidin-4-yl)methyl) piperidin-4-yl)methyl)carbamate (6)

To a solution of benzyl ((1-(piperidin-4-ylmethyl)piperidin-4-yl)methyl)carbamate (150 mg, 392.73 μmol, HCl salt) and 2-(2,6-dioxopiperidin-3-yl)-4-fluoroisoindoline-1,3-dione (130.18 mg, 471.28 μmol) in DMSO (2 mL) was added TEA (119.22 mg, 1.18 mmol, 163.99 μL), the mixture was stirred at 100° C. for 16 h. LCMS showed a peak (45%) with desired mass. The reaction mixture was diluted with H₂O (10 mL), extracted with EtOAc (10 mL×3). The combined organic layers were washed with brine (20 mL×2), dried over Na₂SO₄, filtered. The filtrate was concentrated under reduced pressure. The residue was purified by flash silica gel chromatography (Biotage; 20 g SepaFlash® Silica Flash Column, Eluent of 20˜100% EtOAc/Petroleum ether to 10% Methanol/EtOAc gradient @40 mL/min) to afford benzyl ((1-((1-(2-(2,6-dioxopiperidin-3-yl)-1,3-dioxoisoindolin-4-yl)piperidin-4-yl)methyl)piperidin-4-yl)methyl)carbamate (200 mg, 309.13 μmol, 78.71% yield, 93% purity) as a yellow solid. MS(M+H)⁺=602.4

### Step 4. Synthesis of 4-(4-((4-(aminomethyl)piperidin-1-yl)methyl)piperidin-1-yl)-2-(2,6-dioxopiperidin-3-yl)isoindoline-1,3-dione (7)

A mixture of benzyl ((1-((1-(2-(2,6-dioxopiperidin-3-yl)-1,3-dioxoisoindolin-4-yl)piperidin-4-yl)methyl)piperidin-4-yl)methyl)carbamate (100 mg, 166.20 μmol) in TFA (1 mL) was stirred at 60° C. for 16 h. LCMS showed a peak (78%) with desired mass. The mixture was concentrated under reduced pressure to afford 4-(4-((4-(aminomethyl)piperidin-1-yl)methyl)piperidin-1-yl)-2-(2,6-dioxopiperidin-3-yl)isoindoline-1,3-dione (90 mg, crude, TFA salt) as a brown oil, which was used into the next step directly. MS(M+H)⁺=468.2

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1-((1-(2-(2,6-dioxopiperidin-3-yl)-1,3-dioxoisoindolin-4-yl)piperidin-4-yl)methyl)piperidin-4-yl)methyl)-3-methoxybenzamide (Compound 53)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (69.24 mg, 154.75 μmol) in DMF (2 mL) were added HATU (88.26 mg, 232.13 μmol) and DIPEA (60.00 mg, 464.25 μmol, 80.86 μL), the mixture was stirred at 20° C. for 0.5 h. Then 4-(4-((4-(aminomethyl)piperidin-1-yl)methyl)piperidin-1-yl)-2-(2,6-dioxopiperidin-3-yl)isoindoline-1,3-dione (90 mg, 154.75 μmol, TFA salt) was added and the resulting mixture was stirred at 20° C. for 16 h. LCMS showed a peak (47%) with desired mass. The reaction mixture was diluted with H₂O (5 mL), extracted with EtOAc (5 mL×3). The combined organic layers were washed with brine (10 mL×2), dried over Na₂SO₄, filtered. The filtrate was concentrated under reduced pressure. The residue was purified by prep-TLC (SiO₂, DCM:MeOH=10:1) and re-purified by reversed-phase HPLC (column: Waters Xbridge 150*25 mm*5 μm; mobile phase: [water(NH₄HCO₃)-ACN]; B %: 46%-76%, 8 min). The eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-((1-((1-(2-(2,6-dioxopiperidin-3-yl)-1,3-dioxoisoindolin-4-yl)piperidin-4-yl)methyl)piperidin-4-yl)methyl)-3-methoxybenzamide (28.7 mg, 31.36 μmol, 20.26% yield, 98% purity) as a yellow solid. MS(M+H)⁺=897.4

¹H NMR (400 MHz, DMSO-d₆) δ=11.09 (s, 1H), 8.39 (t, J=5.7 Hz, 1H), 8.29-8.24 (m, 2H), 7.97 (s, 1H), 7.70-7.64 (m, 1H), 7.53-7.47 (m, 2H), 7.32 (t, J=6.8 Hz, 2H), 5.12-5.04 (m, 1H), 4.81-4.71 (m, 1H), 4.04 (t, J=14.1 Hz, 2H), 3.93 (s, 3H), 3.72-3.63 (m, 2H), 3.35 (s, 3H), 3.16 (t, J=5.8 Hz, 2H), 2.95-2.79 (m, 5H), 2.63-2.53 (m, 2H), 2.18-2.12 (m, 2H), 2.06-1.90 (m, 3H), 1.89-1.75 (m, 4H), 1.74-1.50 (m, 10H), 1.37-1.11 (m, 4H).

### Example 54. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-((1-(2-(2,6-dioxopiperidin-3-yl)-1,3-dioxoisoindolin-4-yl)piperidin-3-yl)methyl)piperidin-4-yl)-3-methoxybenzamide (Compound 54)

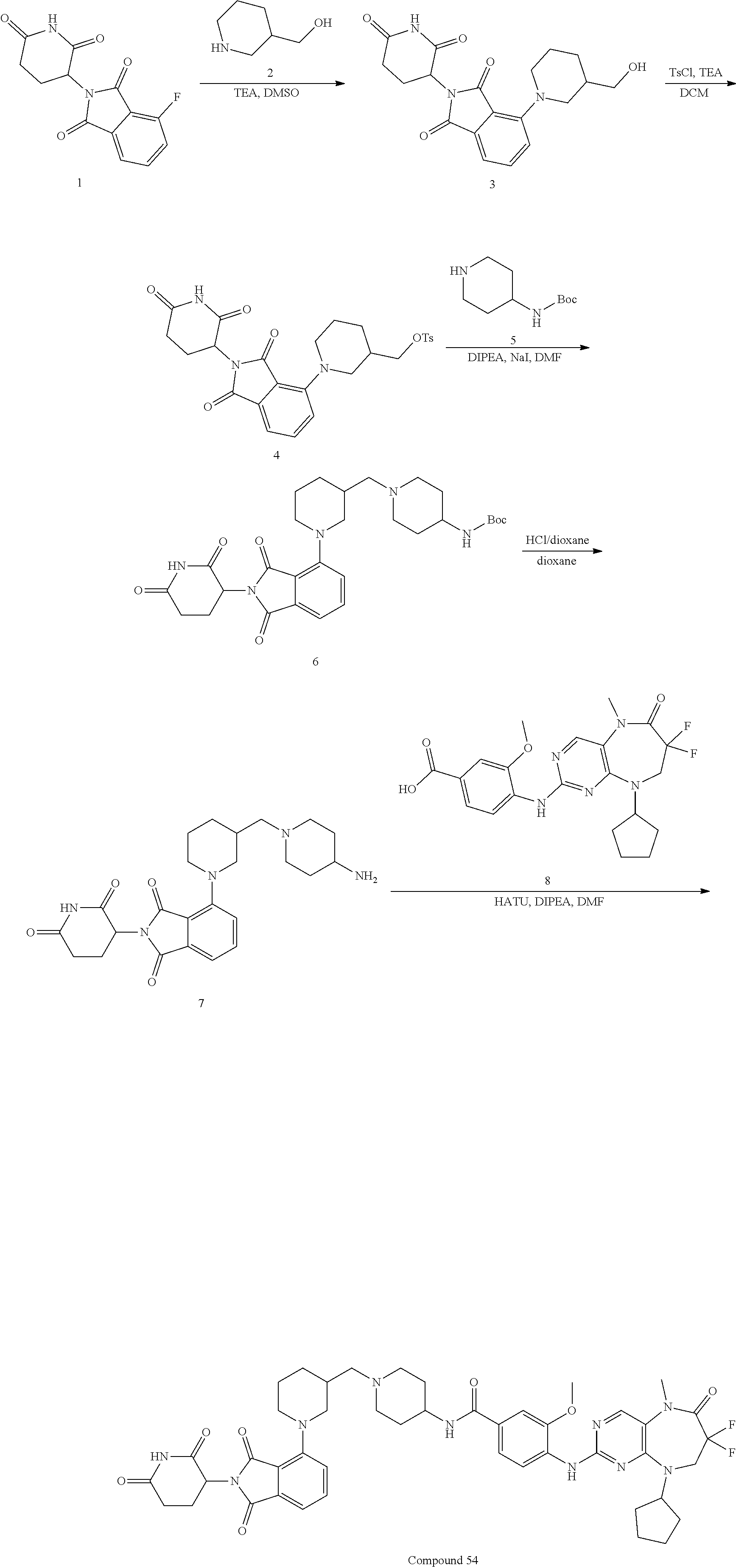

The compound 54 was synthesized by the method described in the scheme similar to the method described in Example 50.

MS(M+H)⁺=883.0, ¹H NMR (400 MHz, DMSO-d₆) δ=11.09 (s, 1H), 8.32-8.24 (m, 2H), 8.06 (d, J=7.3 Hz, 1H), 7.96 (s, 1H), 7.70 (t, J=7.8 Hz, 1H), 7.53-7.45 (m, 2H), 7.38-7.30 (m, 2H), 5.16-5.06 (m, 1H), 4.83-4.72 (m, 1H), 4.05 (t, J=14.0 Hz, 2H), 3.94 (s, 3H), 3.83-3.70 (m, 2H), 3.68-3.59 (m, 1H), 3.33 (s, 3H), 3.32-3.29 (m, 1H), 3.06-2.97 (m, 1H), 2.94-2.83 (m, 2H), 2.82-2.74 (m, 1H), 2.66-2.56 (m, 2H), 2.27-2.15 (m, 2H), 2.08-1.89 (m, 6H), 1.82-1.69 (m, 6H), 1.67-1.52 (m, 7H), 1.18-1.04 (m, 1H).

### Example 55. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-(2-(2,6-dioxopiperidin-3-yl)-1,3-dioxoisoindolin-4-yl)piperidin-3-yl)ethyl)piperidin-4-yl)-3-methoxybenzamide (Compound 55)

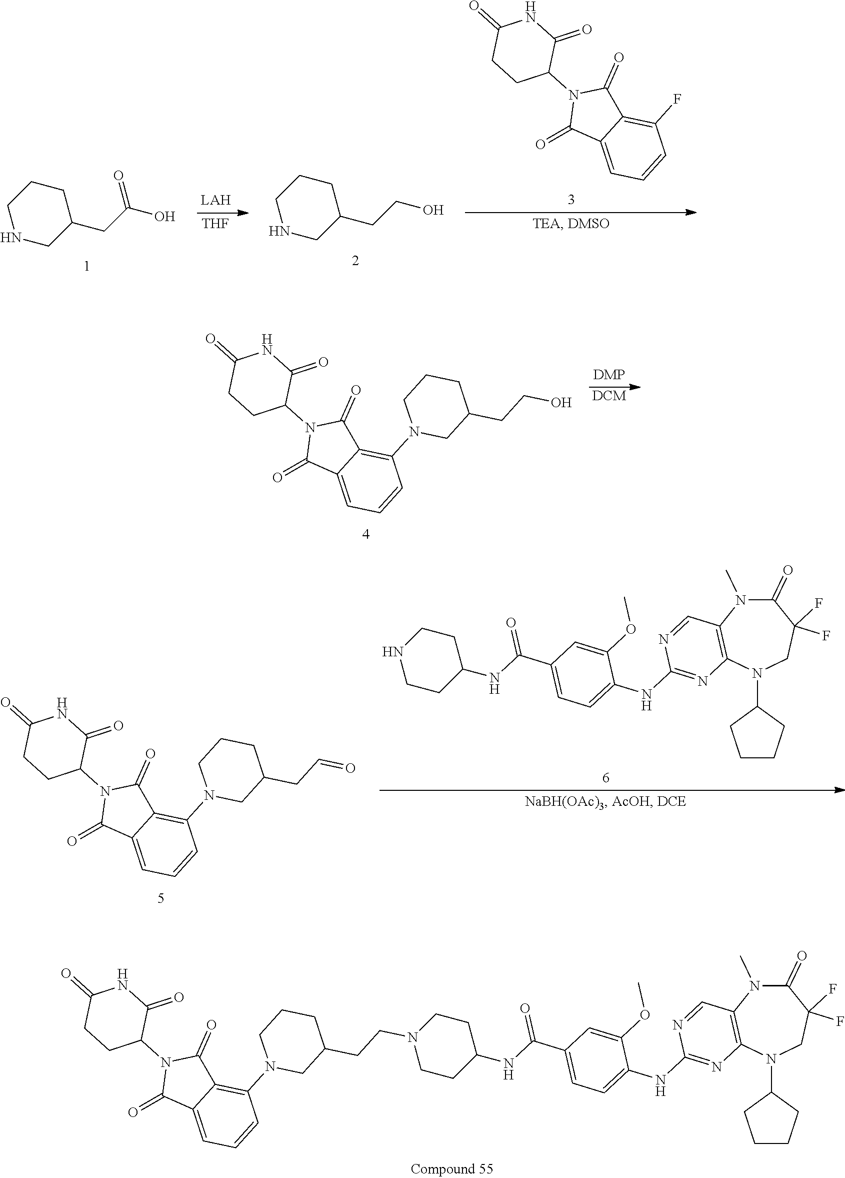

### Step 1. Synthesis of 2-(piperidin-3-yl)ethan-1-ol (2)

To a mixture of LiAlH₄ (238.57 mg, 6.29 mmol) in THF (5 mL) was added a solution of 2-(piperidin-3-yl)acetic acid (0.3 g, 2.10 mmol) in THF (5 mL) at 0° C. The mixture was stirred at 60° C. for 12 h. LCMS showed desired mass was detected. The mixture was quenched with saturated potassium sodium tartrate solution (20 mL). The mixture was stirred at 25° C. for 10 minutes and then filtered. The filtrate was concentrated under reduced pressure to afford 2-(piperidin-3-yl)ethan-1-ol (0.3 g, crude) as yellow oil. MS(M+H)⁺=130.1

### Step 2. Synthesis of 2-(2,6-dioxopiperidin-3-yl)-4-(3-(2-hydroxyethyl)piperidin-1-yl)isoindoline-1,3-dione (4)

To a solution of 2-(2,6-dioxopiperidin-3-yl)-4-fluoroisoindoline-1,3-dione (513.10 mg, 1.86 mmol) in DMSO (10 mL) were added TEA (704.88 mg, 6.97 mmol, 969.57 μL) and 2-(piperidin-3-yl)ethan-1-ol (0.3 g, 2.32 mmo) at 25° C. The mixture was stirred at 80° C. for 12 h. LCMS showed 80% of desired mass was detected. The reaction mixture was diluted with water (50 mL) and extracted with EtOAc (50 mL×4). The combined organic layers were washed with saturated brine (100 mL), dried over anhydrous sodium sulfate, filtered and concentrated under reduced pressure to give a residue. The residue was purified by flash silica gel chromatography (ISCO®; 40 g SepaFlash® Silica Flash Column, Eluent of 0˜40% petroleum ether:EtOAc/ethanol (v/v=5/1) gradient @60 mL/min) to afford 2-(2,6-dioxopiperidin-3-yl)-4-(3-(2-hydroxyethyl)piperidin-1-yl)isoindoline-1,3-dione (0.89 g, 2.30 mmol, 98.95% yield, 99.5% purity) as yellow oil. MS(M+H)⁺=386.2

### Step 3. Synthesis of 2-(1-(2-(2,6-dioxopiperidin-3-yl)-1,3-dioxoisoindolin-4-yl)piperidin-3-yl)acetaldehyde (5)

To a solution of 2-(2,6-dioxopiperidin-3-yl)-4-(3-(2-hydroxyethyl)piperidin-1-yl)isoindoline-1,3-dione (0.8 g, 2.08 mmol) in DCM (20 mL) was added DMP (1.76 g, 4.15 mmol, 1.29 mL). The mixture was stirred at 25° C. for 2 h. TLC (petroleum ether:EtOAc=1:2) indicated one new spot formed. The mixture was filtered to remove the insoluble solid. The filtrate was concentrated under reduced pressure to give a residue. The residue was purified by flash silica gel chromatography (ISCO®; 40 g SepaFlash® Silica Flash Column, Eluent of 0˜50% petroleum ether:EtOAc gradient @80 mL/min) to afford 2-(1-(2-(2,6-dioxopiperidin-3-yl)-1,3-dioxoisoindolin-4-yl)piperidin-3-yl)acetaldehyde (0.47 g, 950.06 μmol, 45.77% yield, 77.5% purity) as a yellow solid. MS(M+H)⁺=383.9

¹H NMR (400 MHz, CDCl₃) δ=9.83 (s, 1H), 8.02-7.89 (m, 1H), 7.58 (dd, J=7.2, 8.4 Hz, 1H), 7.38 (d, J=7.2 Hz, 1H), 7.22-7.17 (m, 1H), 4.98-4.94 (m, 1H), 3.71-3.57 (m, 2H), 3.05-2.69 (m, 5H), 2.56-2.38 (m, 3H), 2.18-2.10 (m, 1H), 1.97-1.80 (m, 3H), 1.70-1.63 (m, 1H).

### Step 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-(2-(2,6-dioxopiperidin-3-yl)-1,3-dioxoisoindolin-4-yl)piperidin-3-yl)ethyl)piperidin-4-yl)-3-methoxybenzamide (Compound 55)

To a solution of 2-(1-(2-(2,6-dioxopiperidin-3-yl)-1,3-dioxoisoindolin-4-yl)piperidin-3-yl)acetaldehyde (80 mg, 208.66 μmol) in DCE (5 mL) were added NaBH(OAc)₃ (132.67 mg, 625.98 μmol), 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(piperidin-4-yl)benzamide (124.02 mg, 219.09 μmol, HCl) and AcOH (12.53 mg, 208.66 μmol, 11.93 μL). The mixture was stirred at 25° C. for 12 h. LCMS showed ˜75% of desired mass was detected. The mixture was quenched with saturated sodium bicarbonate solution (50 mL) and then extracted with EtOAc (50 mL×3). The combined organic layers were washed with saturated brine (50 mL), dried over anhydrous sodium sulfate, filtered and concentrated under reduced pressure to give a residue. The residue was purified by prep-HPLC (column: Phenomenex Synergi C18 150×25 mm×10 μm; mobile phase: [water(FA)-ACN]; B %: 20%-50%, 10 min) and then by prep-HPLC (column: Waters Xbridge 150×25 mm×5 μm; mobile phase: [water(NH₄HCO₃)-ACN]; B %: 50%-80%, 8 min) followed by lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-(2-(2,6-dioxopiperidin-3-yl)-1,3-dioxoisoindolin-4-yl)piperidin-3-yl)ethyl)piperidin-4-yl)-3-methoxybenzamide (54.6 mg, 59.17 μmol, 28.36% yield, 97.2% purity) as a yellow solid. MS(M+H)⁺=897.2

¹H NMR (400 MHz, DMSO-d₆) δ=11.08 (s, 1H), 8.29-8.23 (m, 2H), 8.10-8.07 (m, 1H), 7.96 (s, 1H), 7.72-7.65 (m, 1H), 7.50-7.44 (m, 2H), 7.37 (d, J=8.3 Hz, 1H), 7.31 (d, J=7.0 Hz, 1H), 5.09 (dd, J=5.2, 12.7 Hz, 1H), 4.82-4.72 (m, 1H), 4.04 (t, J=14.1 Hz, 2H), 3.93 (s, 3H), 3.80-3.63 (m, 3H), 3.36-3.34 (m, 3H), 2.96-2.80 (m, 4H), 2.64-2.52 (m, 5H), 2.47-2.43 (m, 2H), 2.03-1.89 (m, 4H), 1.87-1.71 (m, 6H), 1.63-1.35 (m, 8H), 1.17-1.07 (m, 2H).

### Example 56. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(1-(2-(2,6-dioxopiperidin-3-yl)-1,3-dioxoisoindolin-5-yl)pyrrolidin-3-yl)piperazin-1-yl)-3-methoxybenzamide (Compound 56)

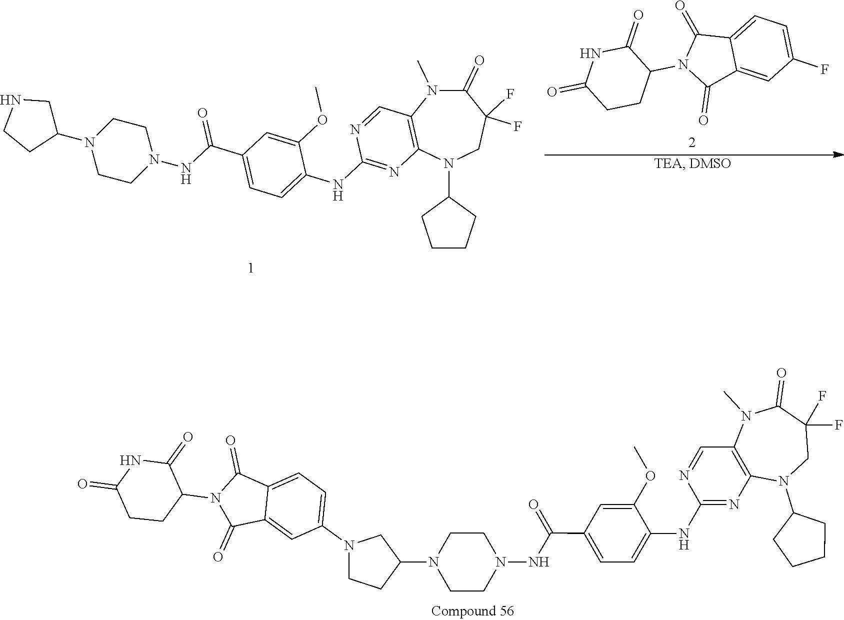

The compound 56 was synthesized by the method described in the scheme similar to the method described in Example 51.

MS(M+H)⁺=856.2, ¹H NMR (400 MHz, DMSO-d₆) δ=11.06 (s, 1H), 9.36 (s, 1H), 8.30-8.23 (m, 2H), 7.97 (s, 1H), 7.65 (d, J=8.4 Hz, 1H), 7.45-7.40 (m, 2H), 6.97 (s, 1H), 6.85 (d, J=8.4 Hz, 1H), 5.06 (dd, J=5.5, 12.6 Hz, 1H), 4.80-4.73 (m, 1H), 4.04 (t, J=14.3 Hz, 2H), 3.93 (s, 3H), 3.75-3.66 (m, 1H), 3.63-3.54 (m, 1H), 3.43-3.38 (m, 1H), 3.29-3.28 (m, 3H), 3.23-3.20 (m, 1H), 3.02-2.87 (m, 6H), 2.63-2.59 (m, 6H), 2.28-2.20 (m, 1H), 2.03-1.86 (m, 4H), 1.74-1.57 (m, 6H).

### Example 57. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(1-(2-(2,6-dioxopiperidin-3-yl)-1,3-dioxoisoindolin-4-yl)piperidin-3-yl)propyl)piperidin-4-yl)-3-methoxybenzamide (Compound 57)

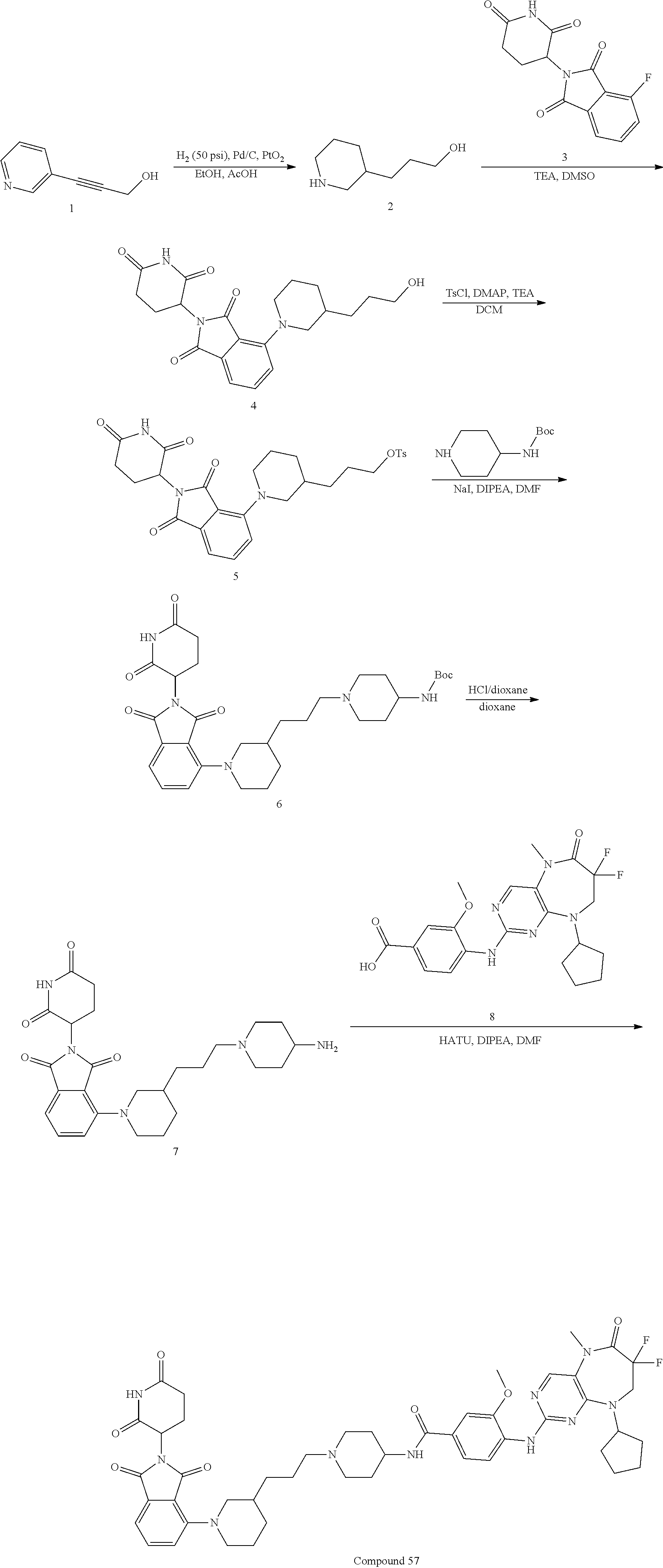

### Step 1. Synthesis of 3-(piperidin-3-yl)propan-1-ol (2)

To a solution of 3-(pyridin-3-yl)prop-2-yn-1-ol (0.5 g, 3.76 mmol) in AcOH (10 mL) and EtOH (10 mL) were added Pd/C (2 g, 18.78 mmol, 10% purity) and PtO₂ (426.37 mg, 1.88 mmol, 100% purity) at 25° C. The mixture was stirred at 50° C. for 24 h under H₂ (50 psi) atmosphere. LCMS a main peak of desired mass was detected. The mixture was filtered to remove the catalyst. The filtrate was concentrated under reduced pressure to afford 3-(piperidin-3-yl)propan-1-ol (0.6 g, crude) as yellow oil. MS(M+H)⁺=144.2

### Step 2. Synthesis of 2-(2,6-dioxopiperidin-3-yl)-4-(3-(3-hydroxypropyl)piperidin-1-yl)isoindoline-1,3-dione (4)

To a solution of 2-(2,6-dioxopiperidin-3-yl)-4-fluoroisoindoline-1,3-dione (0.9 g, 3.26 mmol) in DMSO (10 mL) were added TEA (989.11 mg, 9.77 mmol, 1.36 mL) and 3-(piperidin-3-yl)propan-1-ol (466.67 mg, 3.26 mmol) at 25° C. The mixture was stirred at 90° C. for 3 h. LCMS showed 44% of desired mass was detected. The mixture was diluted with water (50 mL) and extracted with EtOAc (50 mL×3). The combined organic layers were washed with saturated brine (50 mL), dried over anhydrous sodium sulfate, filtered and concentrated under reduced pressure to give a residue. The residue was purified by flash silica gel chromatography (ISCO®; 20 g SepaFlash® Silica Flash Column, Eluent of 0˜80% petroleum ether:EtOAc gradient @60 mL/min) to afford 2-(2,6-dioxopiperidin-3-yl)-4-(3-(3-hydroxypropyl)piperidin-1-yl)isoindoline-1,3-dione (0.8 g, 1.83 mmol, 56.24% yield, 91.5% purity) as a yellow solid. MS(M+H)⁺=400.0

¹H NMR (400 MHz, DMSO-d₆) δ=11.08 (s, 1H), 7.67 (dd, J=7.3, 8.3 Hz, 1H), 7.32 (dd, J=7.9, 11.2 Hz, 2H), 5.13-5.05 (m, 1H), 4.38-4.35 (m, 1H), 3.71-3.60 (m, 2H), 3.39 (q, J=6.2 Hz, 2H), 2.94-2.76 (m, 2H), 2.63-2.54 (m, 2H), 2.08-1.98 (m, 1H), 1.85-1.82 (m, 1H), 1.79-1.59 (m, 3H), 1.54-1.39 (m, 2H), 1.28-1.17 (m, 2H), 1.09-0.99 (m, 1H).

### Step 3. Synthesis of 3-(1-(2-(2,6-dioxopiperidin-3-yl)-1,3-dioxoisoindolin-4-yl)piperidin-3-yl)propyl 4-methylbenzenesulfonate (5)

To a solution of 2-(2,6-dioxopiperidin-3-yl)-4-(3-(3-hydroxypropyl)piperidin-1-yl)isoindoline-1,3-dione (0.4 g, 1.00 mmol) in DCM (5 mL) were added TsCl (286.37 mg, 1.50 mmol) and TEA (202.66 mg, 2.00 mmol, 278.76 μL). The mixture was stirred at 25° C. for 12 h. LCMS showed ˜50% of 2-(2,6-dioxopiperidin-3-yl)-4-(3-(3-hydroxypropyl)piperidin-1-yl)isoindoline-1,3-dione remained and ˜28% of desired mass was detected. TsCl (286.37 mg, 1.50 mmol), DMAP (24.47 mg, 200.28 μmol) and TEA (303.99 mg, 3.00 mmol, 418.15 μL) were added to the mixture. The mixture was stirred for another 4 h at 25° C. TLC (petroleum ether:EtOAc=1:2) indicated one new spot formed. The reaction mixture was diluted with water (50 mL) and extracted with DCM (50 mL×3). The combined organic layers were washed with saturated brine (50 mL), dried over anhydrous sodium sulfate, filtered and concentrated under reduced pressure to give a residue. The residue was purified by flash silica gel chromatography (ISCO®; 20 g SepaFlash® Silica Flash Column, Eluent of 0˜20% petroleum ether:EtOAc/ethanol (v/v=5/1) gradient @80 mL/min) to afford 3-(1-(2-(2,6-dioxopiperidin-3-yl)-1,3-dioxoisoindolin-4-yl)piperidin-3-yl)propyl 4-methylbenzenesulfonate (0.36 g, 617.10 μmol, 61.62% yield, 94.9% purity) as a yellow solid. MS(M+H)⁺=554.1

### Step 4. Synthesis of tert-butyl (1-(3-(1-(2-(2,6-dioxopiperidin-3-yl)-1,3-dioxoisoindolin-4-yl)piperidin-3-yl)propyl)piperidin-4-yl)carbamate (6)

To a solution of 3-(1-(2-(2,6-dioxopiperidin-3-yl)-1,3-dioxoisoindolin-4-yl)piperidin-3-yl)propyl 4-methylbenzenesulfonate (310 mg, 559.95 μmol) in DMF (5 mL) were added tert-butyl piperidin-4-ylcarbamate (134.57 mg, 671.93 μmol), DIPEA (144.74 mg, 1.12 mmol, 195.06 μL) and NaI (41.97 mg, 279.97 μmol) at 25° C. The mixture was stirred at 80° C. for 12 h. TLC (dichloromethane:methanol=10:1) indicated several new spots formed. The reaction mixture was diluted with water (50 mL) and extracted with EtOAc (50 mL×3). The combined organic layers were washed with saturated brine (150 mL), dried over anhydrous sodium sulfate, filtered and concentrated under reduced pressure to give a residue. The residue was purified by flash silica gel chromatography (ISCO®; 20 g SepaFlash® Silica Flash Column, Eluent of 0˜20% petroleum ether/dichloromethane:methanol (v:v=2:1) gradient @80 mL/min) to afford tert-butyl (1-(3-(1-(2-(2,6-dioxopiperidin-3-yl)-1,3-dioxoisoindolin-4-yl)piperidin-3-yl)propyl)piperidin-4-yl)carbamate (0.2 g, 337.97 μmol, 60.36% yield, 98.3% purity) as a yellow solid. MS(M+H)⁺=582.6

### Step 5. Synthesis of 4-(3-(3-(4-aminopiperidin-1-yl)propyl)piperidin-1-yl)-2-(2,6-dioxopiperidin-3-yl)isoindoline-1,3-dione (7)

To a solution of tert-butyl (1-(3-(1-(2-(2,6-dioxopiperidin-3-yl)-1,3-dioxoisoindolin-4-yl)piperidin-3-yl)propyl)piperidin-4-yl)carbamate (240 mg, 412.58 μmol) in dioxane (5 mL) was added HCl/dioxane (4 M, 10 mL). The mixture was stirred at 25° C. for 2 h. TLC (dichloromethane:methanol=10:1) indicated one new spot formed. The mixture was filtered. The filter cake was dried under reduced pressure to give 4-(3-(3-(4-aminopiperidin-1-yl)propyl)piperidin-1-yl)-2-(2,6-dioxopiperidin-3-yl)isoindoline-1,3-dione (0.22 g, crude, HCl) as a yellow solid. MS(M+H)⁺=482.3

¹H NMR (400 MHz, DMSO-d₆) δ=11.09 (s, 1H), 10.84-10.63 (m, 1H), 8.39-8.38 (m, 2H), 7.68 (dd, J=7.3, 8.3 Hz, 1H), 7.34 (t, J=7.8 Hz, 2H), 5.16-5.07 (m, 1H), 3.71-3.58 (m, 2H), 3.57-3.54 (m, 1H), 3.35-3.21 (m, 1H), 3.10-2.79 (m, 6H), 2.62-2.52 (m, 4H), 2.19-1.57 (m, 12H), 1.30-1.19 (m, 2H).

### Step 6. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(1-(2-(2,6-dioxopiperidin-3-yl)-1,3-dioxoisoindolin-4-yl)piperidin-3-yl)propyl)piperidin-4-yl)-3-methoxybenzamide (Compound 57)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (80 mg, 178.80 μmol) in DMF (3 mL) were added HATU (101.98 mg, 268.20 μmol) and DIPEA (69.32 mg, 536.39 mol, 93.43 μL). Then 4-(3-(3-(4-aminopiperidin-1-yl)propyl)piperidin-1-yl)-2-(2,6-dioxopiperidin-3-yl)isoindoline-1,3-dione (100.04 mg, crude, HCl) was added to the mixture after 0.5 h. The mixture was stirred at 25° C. for 2 h. LCMS showed ˜70% of desired mass was detected. The reaction mixture was diluted with water (50 mL) and extracted with EtOAc (50 mL×3). The combined organic layers were washed with saturated brine (50 mL), dried over anhydrous sodium sulfate, filtered and concentrated under reduced pressure to give a residue. The residue was purified by prep-HPLC (column: Phenomenex luna C18 150×40 mm×15 μm; mobile phase: [water(FA)-ACN]; B %: 23%-53%, 10 min) and then prep-HPLC (column: Waters Xbridge 150×25 mm×5 μm; mobile phase: [water(NH₄HCO₃)-ACN]; B %: 54%-84%, 10 min) followed by lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(1-(2-(2,6-dioxopiperidin-3-yl)-1,3-dioxoisoindolin-4-yl)piperidin-3-yl)propyl)piperidin-4-yl)-3-methoxybenzamide (58.2 mg, 63.57 μmol, 35.55% yield, 99.5% purity) as a yellow solid. MS(M+H)⁺=911.3

¹H NMR (400 MHz, DMSO-d₆) δ=11.10 (br s, 1H), 8.29-8.24 (m, 2H), 8.10 (d, J=7.7 Hz, 1H), 7.97 (s, 1H), 7.67 (t, J=7.9 Hz, 1H), 7.52-7.46 (m, 2H), 7.39-7.30 (m, 2H), 5.10 (dd, J=5.6, 12.7 Hz, 1H), 4.83-4.73 (m, 1H), 4.05 (t, J=14.1 Hz, 2H), 3.93 (s, 3H), 3.80-3.61 (m, 3H), 3.33-3.32 (m, 3H), 2.93-2.78 (m, 4H), 2.62-2.55 (m, 5H), 2.29-2.23 (m, 2H), 2.06-2.00 (m, 1H), 1.96-1.89 (m, 4H), 1.86-1.82 (m, 1H), 1.79-1.67 (m, 6H), 1.60-1.45 (m, 7H), 1.27-1.20 (m, 2H), 1.11-1.00 (m, 1H).

### Example 58. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(3-(1-(2-(2,6-dioxopiperidin-3-yl)-1-oxoisoindolin-4-yl)piperidin-4-yl)propanoyl)piperazin-1-yl)-3-methoxybenzamide (Compound 58)

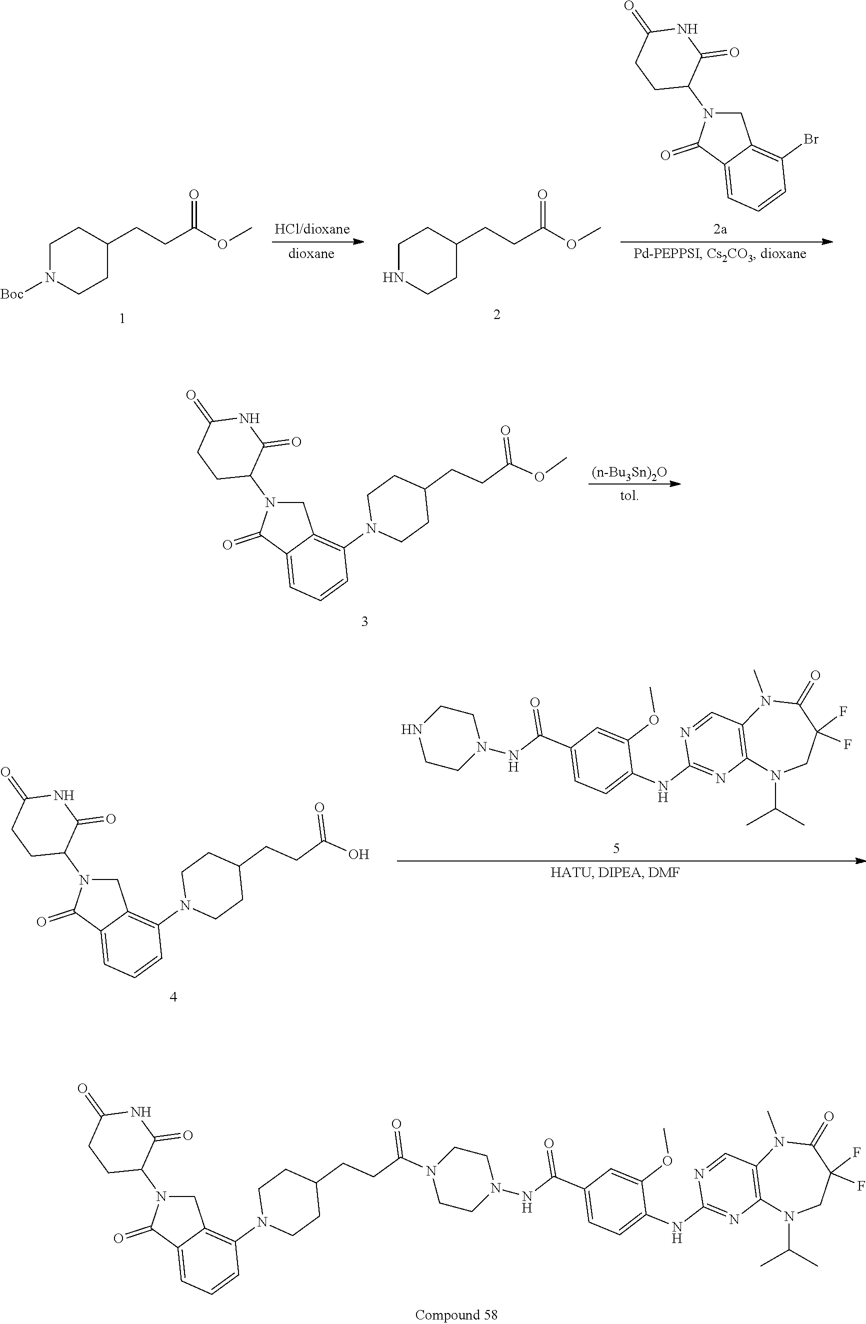

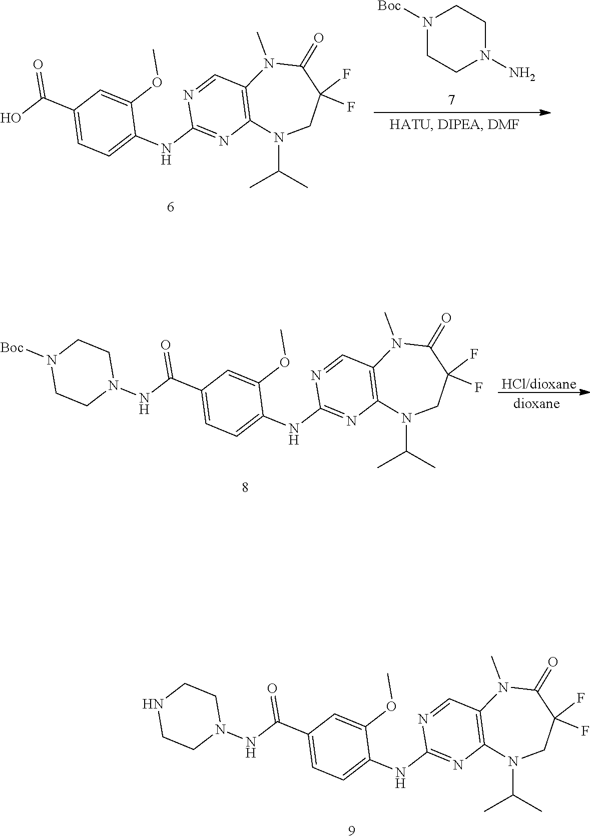

### Step 1. Synthesis of tert-butyl 4-(4-((7,7-difluoro-9-isopropyl-5-methyl-6-oxo-6,7,8,9-tetrahydro-5H-pyrimido[4,5-b][1,4]diazepin-2-yl)amino)-3-methoxybenzamido)piperazine-1-carboxylate (8)

To a solution of 4-((7,7-difluoro-9-isopropyl-5-methyl-6-oxo-6,7,8,9-tetrahydro-5H-pyrimido[4,5-b][1, 4]diazepin-2-yl)amino)-3-methoxybenzoic acid (400 mg, 949.22 μmol) in DMF (5 mL) were added DIPEA (368.04 mg, 2.85 mmol, 496.01 μL) and HATU (541.38 mg, 1.42 mmol). The mixture was stirred at 25° C. for 10 min. Then tert-butyl 4-aminopiperazine-1-carboxylate (210.15 mg, 1.04 mmol) was added and the resulting mixture was stirred at 25° C. for 1 h. LCMS showed a peak (61%) with desired mass. The mixture solution was poured into water (40 mL) and extracted with EtOAc (20 mL×4). The combined organic layers were dried over Na₂SO₄ and concentrated. The crude product was purified by prep-HPLC (column: Phenomenex luna C18 150×40 mm×15 μm; mobile phase: [water(FA)-ACN]; B %: 35%-65%, 10 min) and the eluent was lyophilized to afford tert-butyl 4-(4-((7,7-difluoro-9-isopropyl-5-methyl-6-oxo-6,7,8,9-tetrahydro-5H-pyrimido[4,5-b][1,4]diazepin-2-yl)amino)-3-methoxybenzamido)piperazine-1-carboxylate (260 mg, 417.10 μmol, 43.94% yield, 97% purity) as a white solid, which was used for the next step directly. MS(M+H)⁺=605.4

### Step 2. Synthesis of 4-((7,7-difluoro-9-isopropyl-5-methyl-6-oxo-6,7,8,9-tetrahydro-5H-pyrimido[4,5-b][1,4]diazepin-2-yl)amino)-3-methoxy-N-(piperazin-1-yl)benzamide (9)

To a solution of tert-butyl 4-(4-((7,7-difluoro-9-isopropyl-5-methyl-6-oxo-6,7,8,9-tetrahydro-5H-pyrimido[4,5-b][1,4]diazepin-2-yl)amino)-3-methoxybenzamido)piperazine-1-carboxylate (220 mg, 363.85 μmol) in dioxane (2 mL) was added HCl/dioxane (4 M, 24.44 mL) at 25° C. The resulting mixture was stirred at 25° C. for 0.5 hr. LCMS showed the starting material was consumed completely and a main peak with desired mass. The mixture solution was concentrated under reduced pressure to afford 4-((7,7-difluoro-9-isopropyl-5-methyl-6-oxo-6,7,8,9-tetrahydro-5H-pyrimido[4,5-b][1, 4]diazepin-2-yl)amino)-3-methoxy-N-(piperazin-1-yl)benzamide (200 mg, crude, HCl salt) as a brown solid, which was used for the next step directly. MS(M+H)⁺=505.3

### Step 3. Synthesis of methyl 3-(piperidin-4-yl)propanoate (2)

To a solution of tert-butyl 4-(3-methoxy-3-oxopropyl)piperidine-1-carboxylate (1 g, 3.69 mmol) in dioxane (2 mL) was added HCl/dioxane (4 M, 37.50 mL) at 25° C. The resulting mixture was stirred at 25° C. for 0.5 hr. TLC (petroleum ether:EtOAc=3:1; Rf=0) showed the starting material was consumed completely. The mixture solution was concentrated under reduced pressure to afford methyl 3-(piperidin-4-yl)propanoate (760 mg, crude, HCl salt) as white solid, which was used for the next step directly. MS(M+H)⁺=172.2

### Step 4. Synthesis of methyl 3-(1-(2-(2,6-dioxopiperidin-3-yl)-1-oxoisoindolin-4-yl)piperidin-4-yl)propanoate (3)

To a solution of methyl 3-(piperidin-4-yl)propanoate (208.89 mg, crude, HCl salt), 3-(4-bromo-1-oxoisoindolin-2-yl)piperidine-2,6-dione (250 mg, 773.65 μmol) and Cs₂CO₃ (756.21 mg, 2.32 mmol) in dioxane (10 mL) was added Pd-PEPPSI (37.63 mg, 38.68 μmol) at 25° C. under N₂ atmosphere. The mixture was stirred at 100° C. for 12 h under N₂ atmosphere. LCMS showed the starting material was consumed completely and a main peak with desired mass. The mixture was diluted with EtOAc (40 mL) and washed with brine (10 mL×3). The organic layer was dried over Na₂SO₄, filtered and concentrated under reduced pressure. The residue was purified by flash silica gel chromatography (12 g SepaFlash® Silica Flash Column, Eluent of 0˜60% EtOAc/Petroleum ether gradient @60 mL/min) to afford methyl 3-(1-(2-(2,6-dioxopiperidin-3-yl)-1-oxoisoindolin-4-yl)piperidin-4-yl)propanoate (0.2 g, crude) as white solid, which was used for the next step directly. MS(M+H)⁺=414.2

### Step 5. Synthesis of 3-(1-(2-(2,6-dioxopiperidin-3-yl)-1-oxoisoindolin-4-yl)piperidin-4-yl)propanoic acid (4)

A mixture of methyl 3-(1-(2-(2,6-dioxopiperidin-3-yl)-1-oxoisoindolin-4-yl)piperidin-4-yl)propanoate (0.2 g, 483.72 μmol) and tributyl(tributylstannyloxy)stannane (1.17 g, 1.93 mmol, 999.13 μL) in toluene (5 mL) was stirred at 110° C. for 16 hr. LCMS showed the starting material was consumed completely and a peak (11%) with desired mass. The mixture was poured into KF (2.5M, 30 mL) and extracted with EtOAc (20 mL×5). The combined organic phase was washed with brine (20 mL×3), dried over Na₂SO₄, filtered and concentrated under reduced pressure. The residue was purified by flash silica gel chromatography (12 g SepaFlash® Silica Flash Column, Eluent of 0˜100% EtOAc/Petroleum ether gradient @60 mL/min; Eluent of 0˜50% Methanol/EtOAc @60 mL/min) and re-purified by prep-HPLC (column: Phenomenex Synergi Polar-RP 100×25 mm×4 μm; mobile phase: [water(TFA)-ACN]; B %: 18%-38%, 7 min), and the eluent was lyophilized to afford 3-(1-(2-(2,6-dioxopiperidin-3-yl)-1-oxoisoindolin-4-yl)piperidin-4-yl)propanoic acid (100 mg, crude) as white solid, which was used for the next step directly. MS(M+H)⁺=400.2

### Step 6. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(3-(1-(2-(2,6-dioxopiperidin-3-yl)-1-oxoisoindolin-4-yl)piperidin-4-yl)propanoyl)piperazin-1-yl)-3-methoxybenzamide (Compound 58)

To a solution of 3-(1-(2-(2,6-dioxopiperidin-3-yl)-1-oxoisoindolin-4-yl)piperidin-4-yl)propanoic acid (80 mg, crude) in DMF (2 mL) was added HATU (114.23 mg, 300.42 μmol) and DIPEA (77.65 mg, 600.84 μmol, 104.65 μL). The mixture was stirred at 25° C. for 10 min. Then 4-((7,7-difluoro-9-isopropyl-5-methyl-6-oxo-6,7,8,9-tetrahydro-5H-pyrimido[4,5-b][1, 4]diazepin-2-yl)amino)-3-methoxy-N-(piperazin-1-yl)benzamide (86.52 mg, crude, HCl salt) was added and the resulting mixture was stirred at 25° C. for 2 h. LCMS showed a peak (58%) with desired mass. The mixture was diluted with EtOAc (30 mL) and concentrated under reduced pressure. The residue was purified by flash silica gel chromatography (4 g SepaFlash® Silica Flash Column, Eluent of 0˜100% EtOAc/Petroleum ether gradient @60 mL/min; Eluent of 0˜50% Methanol/EtOAc @60 mL/min) followed by prep-HPLC (column: Waters Xbridge 150×25 mm×5 μm; mobile phase: [water(NH₄HCO₃)-ACN]; B %: 34%-64%, 8 min) and the eluent was lyophilized to afford 4-((7,7-difluoro-9-isopropyl-5-methyl-6-oxo-6,7,8,9-tetrahydro-5H-pyrimido[4,5-b][1, 4]diazepin-2-yl)amino)-N-(4-(3-(1-(2-(2,6-dioxopiperidin-3-yl)-1-oxoisoindolin-4-yl)piperidin-4-yl)propanoyl)piperazin-1-yl)-3-methoxybenzamide (50.9 mg, 53.43 μmol, 26.68% yield, 93% purity) as white solid. MS(M+H)⁺=886.3

¹H NMR (400 MHz, DMSO-d₆) δ=11.05-10.89 (m, 1H), 9.43 (s, 1H), 8.31 (d, J=8.3 Hz, 1H), 8.22 (s, 1H), 7.89 (s, 1H), 7.48-7.37 (m, 3H), 7.30 (d, J=6.9 Hz, 1H), 7.17 (d, J=7.5 Hz, 1H), 5.11 (dd, J=5.1, 13.1 Hz, 1H), 4.87 (q, J=6.8 Hz, 1H), 4.49-4.37 (m, 1H), 4.34-4.23 (m, 1H), 4.03 (t, J=13.4 Hz, 2H), 3.93 (s, 3H), 3.62-3.52 (m, 4H), 3.42-3.37 (m, 2H), 3.32 (s, 3H), 2.98-2.81 (m, 5H), 2.76-2.65 (m, 2H), 2.63-2.56 (m, 1H), 2.48-2.35 (m, 3H), 2.05-1.93 (m, 1H), 1.80 (d, J=11.6 Hz, 2H), 1.57-1.47 (m, 2H), 1.45-1.36 (m, 1H), 1.35-1.26 (m, 2H), 1.24 (d, J=6.6 Hz, 6H).

### Example 59. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(3-(1-(2-(2,6-dioxopiperidin-3-yl)-1-oxoisoindolin-5-yl)piperidin-4-yl)propanoyl)piperazin-1-yl)-3-methoxybenzamide (Compound 59)

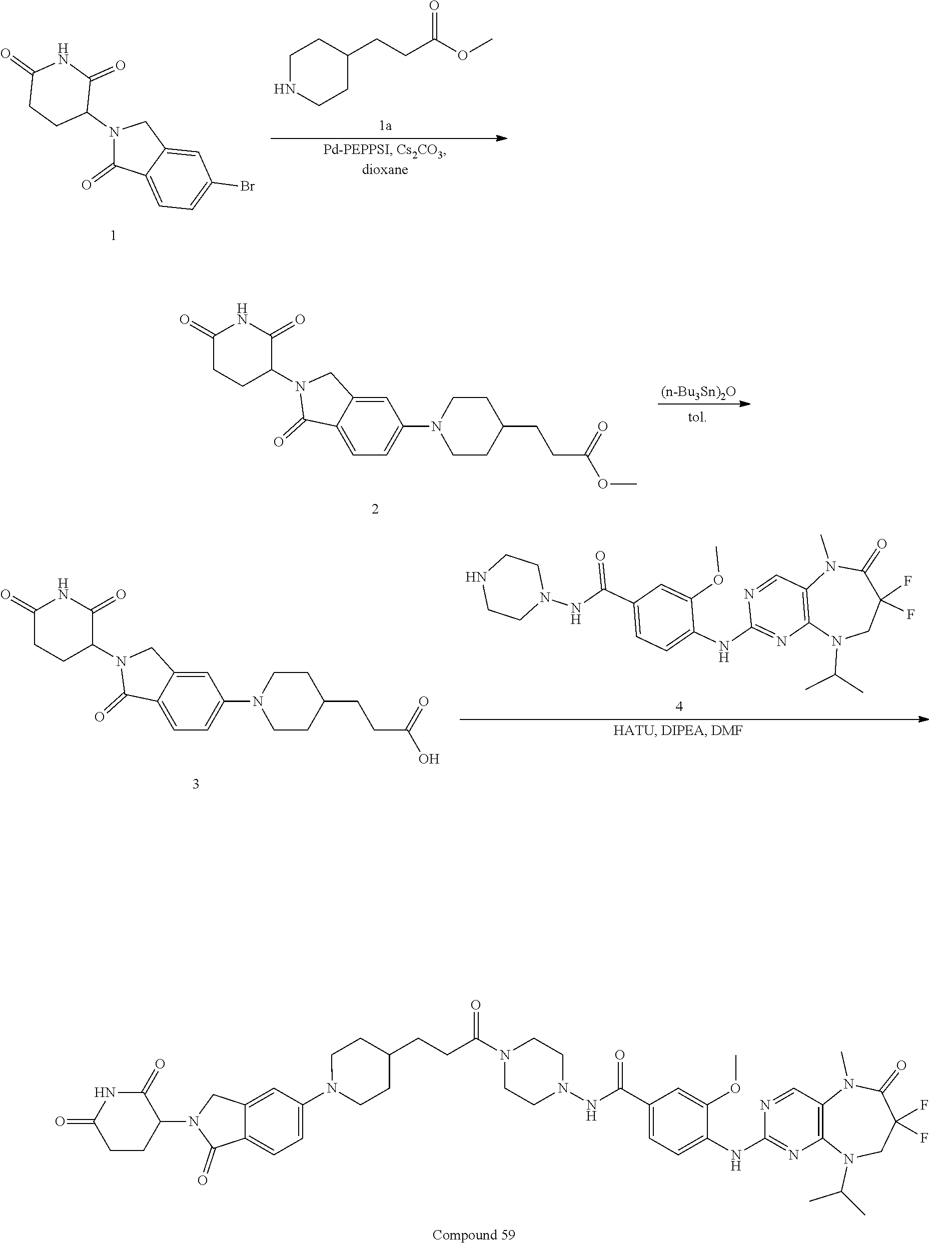

The compound 59 was synthesized by the method described in the scheme similar to the method described in Example 58.

MS(M+H)⁺=886.5, ¹H NMR (400 MHz, CDCl₃) δ=8.48 (d, J=8.5 Hz, 1H), 8.12-7.99 (m, 2H), 7.79-7.67 (m, 2H), 7.41 (s, 1H), 7.38-7.27 (m, 1H), 7.08-6.94 (m, 2H), 6.88 (d, J=1.5 Hz, 1H), 5.19 (dd, J=5.1, 13.2 Hz, 1H), 5.04-4.92 (m, 1H), 4.47-4.33 (m, 1H), 4.30-4.20 (m, 1H), 3.98 (s, 3H), 3.93-3.78 (m, 6H), 3.71-3.61 (m, 2H), 3.41 (s, 3H), 3.06-2.98 (m, 3H), 2.93-2.77 (m, 4H), 2.48-2.26 (m, 3H), 2.25-2.14 (m, 1H), 1.83 (d, J=12.5 Hz, 2H), 1.72-1.61 (m, 4H), 1.45-1.34 (m, 2H), 1.32 (d, J=6.8 Hz, 6H).

### Example 60. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propyl)piperidin-4-yl)-3-methoxybenzamide (Compound 60)

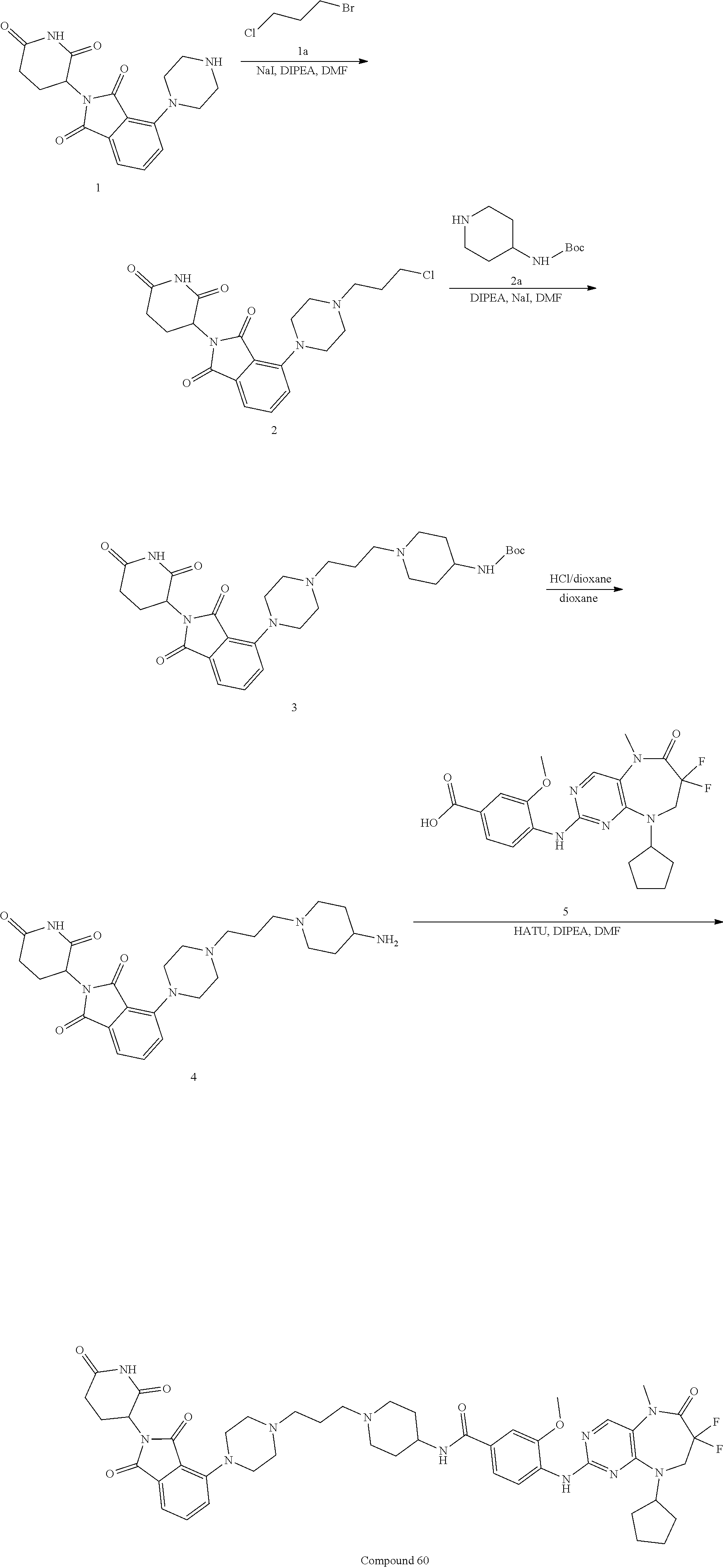

### Step 1. Synthesis of 4-(4-(3-chloropropyl)piperazin-1-yl)-2-(2,6-dioxopiperidin-3-yl)isoindoline-1,3-dione (2)

A mixture 2-(2,6-dioxopiperidin-3-yl)-4-(piperazin-1-yl)isoindoline-1,3-dione (500 mg, 1.46 mmol), 1-bromo-3-chloro-propane (252.93 mg, 1.61 mmol, 158.08 μL), DIEA (566.28 mg, 4.38 mmol, 763.18 μL) and NaI (21.89 mg, 146.05 μmol) in DMF (5 mL) was stirred at 70° C. for 1 hr. LCMS showed the 2-(2,6-dioxopiperidin-3-yl)-4-(piperazin-1-yl)isoindoline-1,3-dione was consumed completely and a main peak with desired mass. The mixture solution was concentrated under reduced pressure to give 4-(4-(3-chloropropyl)piperazin-1-yl)-2-(2,6-dioxopiperidin-3-yl)isoindoline-1,3-dione (611 mg, crude) as brown oil, which was used for the next step directly. MS(M+H)⁺=419.1

### Step 2. Synthesis of tert-butyl (1-(3-(4-(2-(2,6-dioxopiperidin-3-yl)-1,3-dioxoisoindolin-4-yl)piperazin-1-yl)propyl)piperidin-4-yl)carbamate (3)

A mixture of 4-(4-(3-chloropropyl)piperazin-1-yl)-2-(2,6-dioxopiperidin-3-yl)isoindoline-1,3-dione (600 mg, 1.43 mmol), tert-butyl piperidin-4-ylcarbamate (372.94 mg, 1.86 mmol), DIEA (555.38 mg, 4.30 mmol, 748.48 μL) and NaI (21.47 mg, 143.24 μmol) in DMF (5 mL) was stirred at 70° C. for 1 hr. LCMS showed the 4-(4-(3-chloropropyl)piperazin-1-yl)-2-(2,6-dioxopiperidin-3-yl)isoindoline-1,3-dione was consumed completely, and a peak (51%) with desired mass. The mixture was purified by prep-HPLC (column: Waters Xbridge C18 150×50 mm×10 μm; mobile phase: [water(NH₄HCO₃)-ACN]; B %: 28%-58%, 11 min) followed by prep-HPLC (column: Waters Xbridge C18 150×50 mm×10 μm; mobile phase: [water(NH₄HCO₃)-ACN]; B %: 28%-58%, 11 min) and lyophilized to give tert-butyl (1-(3-(4-(2-(2,6-dioxopiperidin-3-yl)-1,3-dioxoisoindolin-4-yl)piperazin-1-yl)propyl)piperidin-4-yl)carbamate (0.3 g, crude) as yellow solid, which was used for the next step directly. MS(M+H)⁺=583.3

### Step 3. Synthesis of 4-(4-(3-(4-aminopiperidin-1-yl)propyl)piperazin-1-yl)-2-(2,6-dioxopiperidin-3-yl)isoindoline-1,3-dione (4)

To a solution of tert-butyl (1-(3-(4-(2-(2,6-dioxopiperidin-3-yl)-1,3-dioxoisoindolin-4-yl)piperazin-1-yl)propyl)piperidin-4-yl)carbamate (0.3 g, 514.85 μmol) in dioxane (1 mL) was added HCl/dioxane (4 M, 27.27 mL) at 25° C. The resulting mixture was stirred at 25° C. for 0.5 hr. LCMS showed the starting material was consumed completely. The mixture solution was concentrated under reduced pressure to give the crude product. The crude product was purified by prep-HPLC (column: Phenomenex Synergi Polar-RP 100×25 mm×4 μm; mobile phase: [water(TFA)-ACN]; B %: 6%-26%, 7 min) and lyophilized to give 4-(4-(3-(4-aminopiperidin-1-yl)propyl)piperazin-1-yl)-2-(2,6-dioxopiperidin-3-yl)isoindoline-1,3-dione (50 mg, 82.13 μmol, 21.31% yield, 98% purity, TFA) as yellow solid, which was used for the next step directly. MS(M+H)⁺=483.1

### Step 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propyl)piperidin-4-yl)-3-methoxybenzamide (Compound 60)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (45 mg, 100.57 μmol) in DMF (2 mL) was added HATU (57.36 mg, 150.86 μmol) and DIPEA (38.99 mg, 301.72 μmol, 52.55 μL). The mixture was stirred at 25° C. for 10 min. To mixture was added 4-(4-(3-(4-aminopiperidin-1-yl)propyl)piperazin-1-yl)-2-(2,6-dioxopiperidin-3-yl)isoindoline-1,3-dione (60.00 mg, 100.57 μmol, TFA). The mixture was stirred at 25° C. for 12 h. LCMS showed the 4-(4-(3-(4-aminopiperidin-1-yl)propyl)piperazin-1-yl)-2-(2,6-dioxopiperidin-3-yl)isoindoline-1,3-dione was consumed completely, and a peak (54%) with desired mass. The mixture was poured into water (10 mL) and extracted with EtOAc (20 mL×3). The combined organic phase was washed with brine (10 mL×3), dried over Na₂SO₄, filtered and concentrated under reduced pressure to give a residue. The residue was purified by prep-TLC (Dichloromethane:Methanol=8:1; Rf=0.4) to give the crude product. The crude product was purified by prep-HPLC (column: Waters Xbridge 150×25 mm×5 μm; mobile phase: [water(NH₄HCO₃)-ACN]; B %: 35%-68%, 8 min) and lyophilized to give 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propyl)piperidin-4-yl)-3-methoxybenzamide (31.4 mg, 33.74 μmol, 33.55% yield, 98% purity) as yellow solid. MS(M+H)⁺=912.1

¹H NMR (400 MHz, DMSO-d₆) δ=11.09 (s, 1H), 8.30-8.23 (m, 2H), 8.10 (d, J=7.6 Hz, 1H), 7.96 (s, 1H), 7.75-7.65 (m, 1H), 7.54-7.44 (m, 2H), 7.35 (t, J=7.5 Hz, 2H), 5.09 (dd, J=5.4, 12.8 Hz, 1H), 4.76 (q, J=8.1 Hz, 1H), 4.04 (t, J=14.1 Hz, 2H), 3.93 (s, 3H), 3.83-3.70 (m, 1H), 3.32-3.26 (m, 7H), 2.93-2.80 (m, 3H), 2.65-2.58 (m, 1H), 2.56-2.49 (m, 6H), 2.42-2.25 (m, 5H), 2.06-2.00 (m, 1H), 1.99-1.89 (m, 4H), 1.82-1.75 (m, 2H), 1.74-1.66 (m, 2H), 1.63-1.57 (m, 6H).

### Example 61. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1-oxoisoindolin-5-yl)piperazin-1-yl)propanoyl)piperidin-4-yl)-2-fluoro-5-methoxybenzamide (Compound 61)

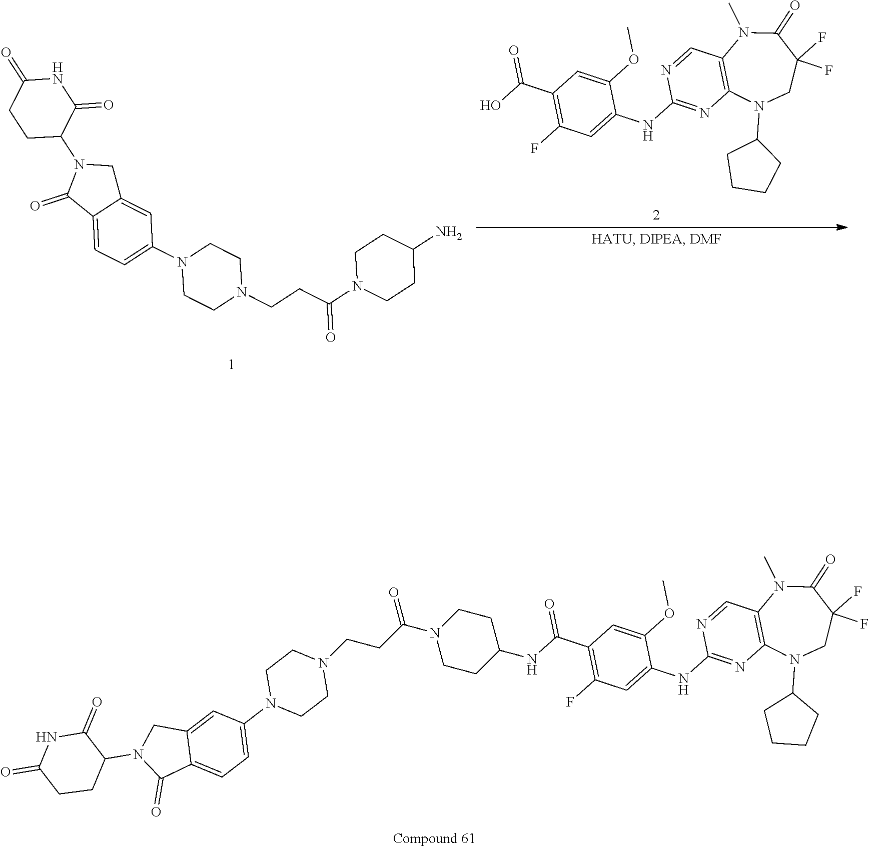 [SMILES: NC1CCN(C(=O)CCN2CCN(c3ccc4c(c3)CN(C3CCC(=O)NC3=O)C4=O)CC2)CC1 ; COc1cc(C(=O)O)c(F)cc1Nc1ncc2c(n1)N(C1CCCC1)CC(F)(F)C(=O)N2C ; COc1cc(C(=O)NC2CCN(C(=O)CCN3CCN(c4ccc5c(c4)CN(C4CCC(=O)NC4=O)C5=O)CC3)CC2)c(F)cc1Nc1ncc2c(n1)N(C1CCCC1)CC(F)(F)C(=O)N2C]

The compound 61 was synthesized by the method described in the scheme similar to the method described in Example 44.

MS(M+H)⁺=930.0, ¹H NMR (400 MHz, DMSO-d₆) δ=10.94 (s, 1H), 8.34-8.21 (m, 2H), 8.07-7.92 (m, 2H), 7.53 (d, J=8.8 Hz, 1H), 7.21 (d, J=6.7 Hz, 1H), 7.12-7.02 (m, 2H), 5.11-5.00 (m, 1H), 4.89-4.77 (m, 1H), 4.38-4.17 (m, 3H), 4.15-4.00 (m, 3H), 3.97-3.85 (m, 4H), 3.34-3.24 (m, 8H), 3.21-3.12 (m, 1H), 2.96-2.84 (m, 1H), 2.79-2.71 (m, 1H), 2.63-2.55 (m, 8H), 2.40-2.32 (m, 1H), 2.00-1.80 (m, 5H), 1.77-1.69 (m, 2H), 1.68-1.57 (m, 4H), 1.55-1.35 (m, 2H).

### Example 62. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(1-(2-(2,6-dioxopiperidin-3-yl)-1,3-dioxoisoindolin-4-yl)piperidin-4-yl)propanoyl)piperidin-4-yl)-3-methoxybenzamide (Compound 62)

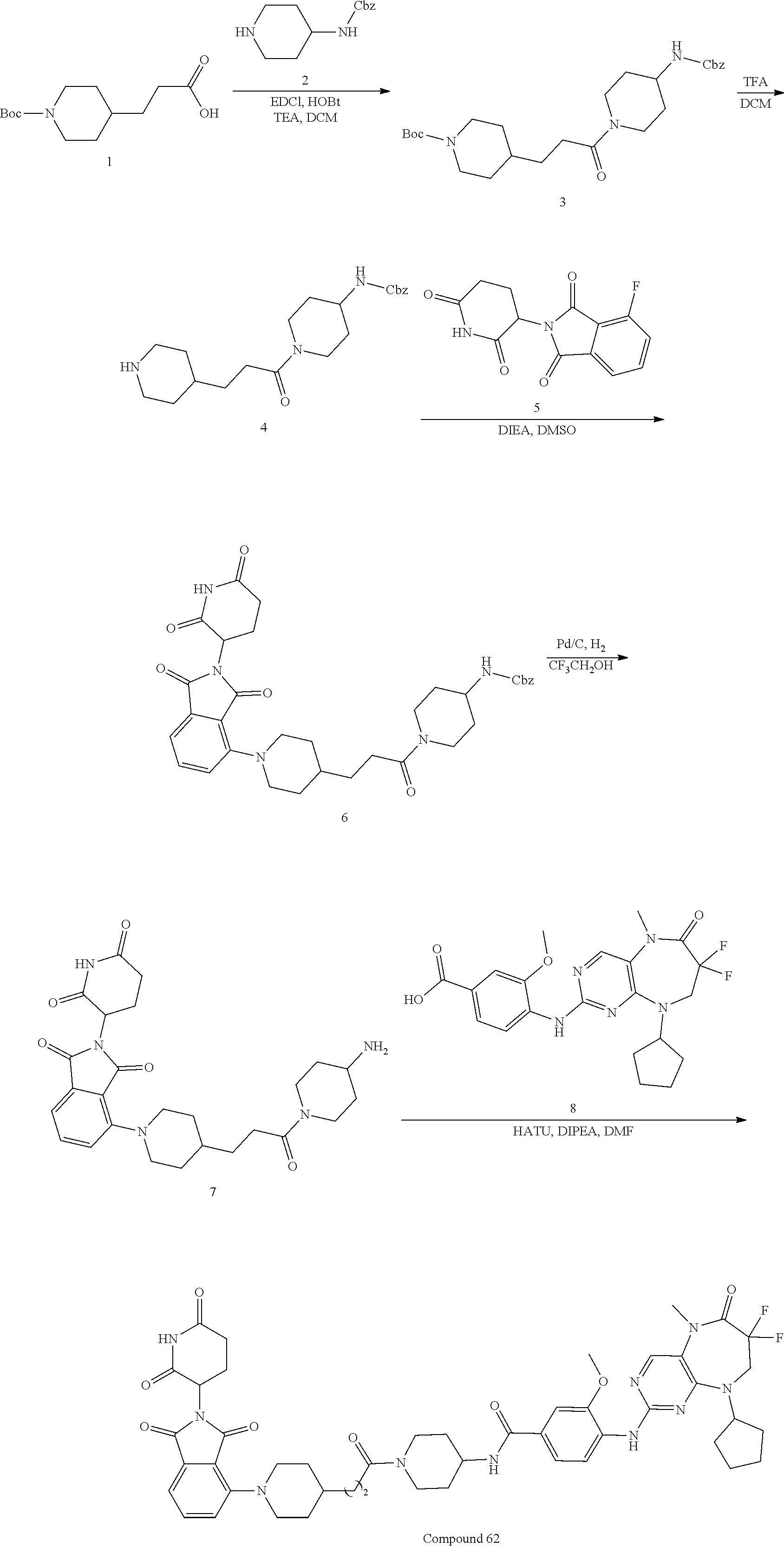

The compound 62 was synthesized by the method described in the scheme similar to the method described in Example 44.

MS(M+H)⁺=925.3, ¹H NMR (400 MHz, DMSO-d₆) δ=11.29-10.81 (m, 1H), 8.36-8.22 (m, 2H), 8.15 (br d, J=7.8 Hz, 1H), 7.96 (s, 1H), 7.67 (dd, J=7.2, 8.3 Hz, 1H), 7.58-7.40 (m, 2H), 7.39-7.22 (m, 2H), 5.09 (dd, J=5.5, 12.9 Hz, 1H), 4.76 (quin, J=8.1 Hz, 1H), 4.48-4.33 (m, 1H), 4.14-4.00 (m, 3H), 3.93 (s, 4H), 3.80-3.60 (m, 2H), 3.43-3.32 (m, 3H), 3.13 (t, J=12.4 Hz, 1H), 2.96-2.77 (m, 3H), 2.72-2.55 (m, 3H), 2.39 (t, J=7.4 Hz, 2H), 2.07-1.99 (m, 1H), 1.99-1.86 (m, 3H), 1.83-1.79 (m, 3H), 1.72-1.69 (m, 2H), 1.61-1.56 (m, 4H), 1.55-1.42 (m, 4H), 1.42-1.25 (m, 3H).

### Example 63. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(1-(2-(2,6-dioxopiperidin-3-yl)-1,3-dioxoisoindolin-4-yl)-4-hydroxypiperidin-4-yl)propanoyl)piperidin-4-yl)-3-methoxybenzamide (Compound 63)

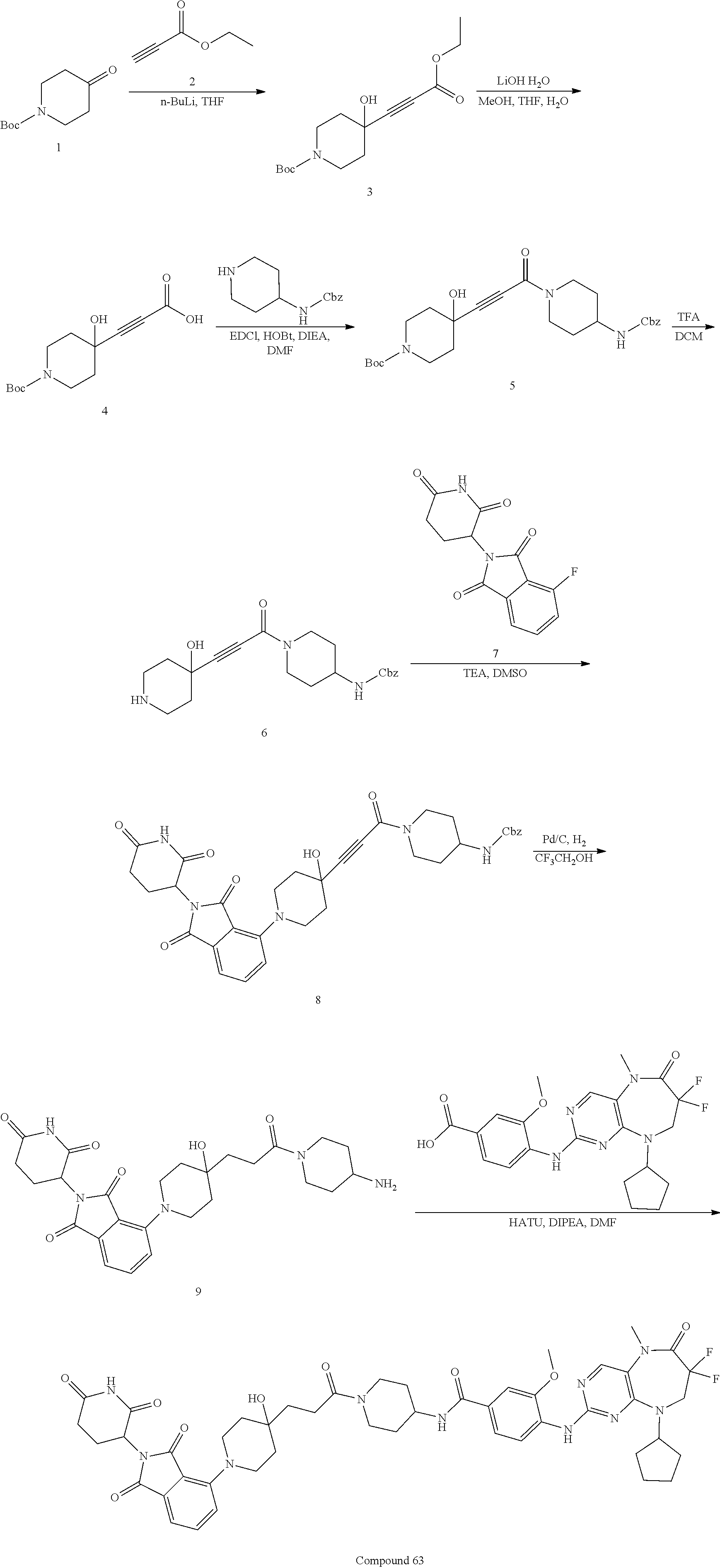

### Step 1. Synthesis of tert-butyl 4-(3-ethoxy-3-oxoprop-1-yn-1-yl)-4-hydroxypiperidine-1-carboxylate (3)

To a solution of ethyl propiolate (1.70 g, 17.33 mmol, 1.70 mL) in THF (10 mL) was slowly added n-BuLi (2.5 M, 9.30 mL) at −70° C. and the mixture was stirred at −70° C. for 15 min. A solution of tert-butyl 4-oxopiperidine-1-carboxylate (1 g, 5.02 mmol) in THF (10 mL) was slowly added at −70° C. and the mixture was stirred at −70° C. for 1 h. LCMS showed 55% of the desired mass was detected. AcOH (1.17 g, 19.51 mmol, 1.12 mL) was added and the mixture was warmed to 20° C. The mixture was diluted with NaHCO₃ (20 mL) and EtOAc (20 mL), and extracted with EtOAc (10 mL×2), the combined organic layer was washed with H₂O (10 mL×3), dried over Na₂SO₄ and filtered. The filtrate was concentrated under reduced pressure. The residue was purified by flash silica gel chromatography (12 g SepaFlash® Silica Flash Column, Eluent of 10˜100% EtOAc/Petroleum ether gradient @50 mL/min) to afford tert-butyl 4-(3-ethoxy-3-oxoprop-1-yn-1-yl)-4-hydroxypiperidine-1-carboxylate (1.37 g, 4.15 mmol, 82.62% yield, 90% purity) as a yellow oil. MS(M+H)⁺=298.3

### Step 2. Synthesis of 3-(1-(tert-butoxycarbonyl)-4-hydroxypiperidin-4-yl)propiolic acid (4)

To a solution of tert-butyl 4-(3-ethoxy-3-oxoprop-1-yn-1-yl)-4-hydroxypiperidine-1-carboxylate (1.17 g, 3.93 mmol) in THF (12 mL), H₂O (0.3 mL) and MeOH (0.3 mL) was added LiOHH₂O (231 mg, 5.50 mmol) and the mixture was stirred at 25° C. for 4 h. LCMS showed 83% of the desired mass was detected after work up. The mixture combined with other batch (0.2 g scale) was concentrated under reduced pressure to afford 3-(1-(tert-butoxycarbonyl)-4-hydroxypiperidin-4-yl)propiolic acid (1.25 g, crude) as a black brown solid. MS(M+H)⁺=270.3

### Step 3. Synthesis of tert-butyl 4-(3-(4-(((benzyloxy)carbonyl)amino)piperidin-1-yl)-3-oxoprop-1-yn-1-yl)-4-hydroxypiperidine-1-carboxylate (5)

To a solution of 3-(1-(tert-butoxycarbonyl)-4-hydroxypiperidin-4-yl)propiolic acid (1.25 g, 4.54 mmol) and benzyl piperidin-4-ylcarbamate (1.17 g, 5.00 mmol) in DMF (20 mL) were added EDCI (1.31 g, 6.81 mmol), HOBt (1.23 g, 9.08 mmol) and DIPEA (1.76 g, 13.63 mmol, 2.37 mL) and the mixture was stirred at 25° C. for 14 h. LCMS showed 63% of the desired mass was detected. The mixture was diluted with H₂O (40 mL) and extracted with EtOAc (15 mL×3), the combined organic layer was washed with brine (10 mL×3), dried over Na₂SO₄ and filtered. The filtrate was concentrated under reduced pressure. The residue was purified by flash silica gel chromatography (12 g SepaFlash® Silica Flash Column, Eluent of 10˜40% EtOAc/Petroleum ether gradient @50 mL/min) to afford tert-butyl 4-(3-(4-(((benzyloxy)carbonyl)amino)piperidin-1-yl)-3-oxoprop-1-yn-1-yl)-4-hydroxy piperidine-1-carboxylate (850 mg, 1.72 mmol, 37.77% yield, 98% purity) as a yellow oil. MS(M+H)⁺=486.2

### Step 4. Synthesis of benzyl (1-(3-(4-hydroxypiperidin-4-yl)propioloyl)piperidin-4-yl)carbamate (6)

To a solution of tert-butyl 4-(3-(4-(((benzyloxy)carbonyl)amino)piperidin-1-yl)-3-oxoprop-1-yn-1-yl)-4-hydroxy piperidine-1-carboxylate (300 mg, 617.83 μmol) in DCM (6 mL) was added TFA (415.80 mg, 3.65 mmol, 270 μL) and the mixture was stirred at 25° C. for 14 h. LCMS showed 75% of the desired mass was detected. The mixture was concentrated under reduced pressure to afford benzyl (1-(3-(4-hydroxypiperidin-4-yl)propioloyl)piperidin-4-yl)carbamate (310 mg, crude, TFA) as a yellow oil. MS(M+H)⁺=386.0

### Step 5. Synthesis of benzyl (1-(3-(1-(2-(2,6-dioxopiperidin-3-yl)-1,3-dioxoisoindolin-4-yl)-4-hydroxypiperidin-4-yl)propioloyl)piperidin-4-yl)carbamate (8)

To a solution of benzyl (1-(3-(4-hydroxypiperidin-4-yl)propioloyl)piperidin-4-yl)carbamate (310 mg, 620.65 μmol, TFA) and 2-(2,6-dioxopiperidin-3-yl)-4-fluoroisoindoline-1,3-dione (163 mg, 590.11 μmol) in DMSO (15 mL) was added TEA (378.04 mg, 3.74 mmol, 520 μL) and the mixture was stirred at 100° C. for 14 h. LCMS showed 92% of the desired mass was detected. The mixture was filtered and the filter cake was washed with EtOAc (30 mL) and H₂O (20 mL). The filtrate was extracted with EtOAc (10 mL×3), the combined organic layer was washed with brine (20 mL×3), dried over Na₂SO₄ and filtered. The filtrate was concentrated under reduced pressure. The residue was purified by flash silica gel chromatography (5 g SepaFlash® Silica Flash Column, Eluent of 20˜98% EtOAc/Petroleum ether gradient @50 mL/min) to afford benzyl (1-(3-(1-(2-(2,6-dioxopiperidin-3-yl)-1,3-dioxoisoindolin-4-yl)-4-hydroxypiperidin-4-yl)propioloyl)piperidin-4-yl)carbamate (0.3 g, 453.50 μmol, 73.07% yield, 97% purity) as a yellow solid. MS(M+H)⁺=642.0

### Step 6. Synthesis of 4-(4-(3-(4-aminopiperidin-1-yl)-3-oxopropyl)-4-hydroxypiperidin-1-yl)-2-(2,6-dioxopiperidin-3-yl)isoindoline-1,3-dione (9)

To a solution of benzyl (1-(3-(1-(2-(2,6-dioxopiperidin-3-yl)-1,3-dioxoisoindolin-4-yl)-4-hydroxypiperidin-4-yl)propioloyl)piperidin-4-yl)carbamate (0.3 g, 467.53 μmol) in CF₃CH₂OH (10 mL) was added Pd/C (50 mg, 10% purity) and the mixture was stirred at 25° C. under H₂ (15 Psi) for 14 h. LCMS showed 46% of the desired mass was detected. The mixture was stirred at 50° C. under H₂ (15 Psi) for 6 h. LCMS showed 80% of the desired mass was detected. The mixture was diluted with THF (10 mL) and then filtered. The filter cake was washed with THF (10 mL) and CF₃CH₂OH (10 mL). The filtrate was concentrated under reduced pressure to afford 4-(4-(3-(4-aminopiperidin-1-yl)-3-oxopropyl)-4-hydroxypiperidin-1-yl)-2-(2,6-dioxopiperidin-3-yl)isoindoline-1,3-dione (240 mg, crude) as a yellow solid. MS(M+H)⁺=512.0

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(1-(2-(2,6-dioxopiperidin-3-yl)-1,3-dioxoisoindolin-4-yl)-4-hydroxypiperidin-4-yl)propanoyl)piperidin-4-yl)-3-methoxybenzamide (Compound 63)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (70 mg, 156.45 μmol) and HATU (89.23 mg, 234.67 μmol) in DMF (1.5 mL) was added DIPEA (60.66 mg, 469.34 μmol, 81.75 μL) and the mixture was stirred at 20° C. for 15 min. 4-(4-(3-(4-aminopiperidin-1-yl)-3-oxopropyl)-4-hydroxypiperidin-1-yl)-2-(2,6-dioxopiperidin-3-yl)isoindoline-1,3-dione (96.04 mg, 187.74 μmol) in DMF (1 mL) was added and the mixture was stirred at 20° C. for 1 h. LCMS showed 79% of the desired mass was detected. The mixture was diluted with H₂O (10 mL) and extracted with EtOAc (10 mL×3), the combined organic layer was washed with brine (10 mL×3), dried over Na₂SO₄ and filtered. The filtrate was concentrated under reduced pressure. The residue was purified by prep-HPLC (column: Waters Xbridge 150*25 mm*5 μm; mobile phase: [water(NH₄HCO₃)-ACN]; B %: 37%-67%, 9 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(1-(2-(2,6-dioxopiperidin-3-yl)-1,3-dioxoisoindolin-4-yl)-4-hydroxypiperidin-4-yl)propanoyl)piperidin-4-yl)-3-methoxybenzamide (44 mg, 44.42 μmol, 28.39% yield, 95% purity) as a yellow solid. MS(M+H)⁺=941.1

¹H NMR (400 MHz, DMSO-d₆) δ=11.08 (s, 1H), 8.30-8.25 (m, 2H), 8.18-8.13 (m, 1H), 7.97 (s, 1H), 7.69-7.65 (m, 1H), 7.51-7.47 (m, 2H), 7.36 (d, J=8.5 Hz, 1H), 7.31 (d, J=7.0 Hz, 1H), 5.11-5.05 (m, 1H), 4.82-4.71 (m, 1H), 4.45-4.36 (m, 2H), 4.09-4.00 (m, 3H), 3.97-3.90 (m, 4H), 3.48-3.39 (m, 2H), 3.32 (s, 3H), 3.26-3.09 (m, 3H), 2.93-2.82 (m, 1H), 2.72-2.63 (m, 1H), 2.62-2.53 (m, 2H), 2.47-2.42 (m, 2H), 2.06-1.79 (m, 5H), 1.74-1.54 (m, 12H), 1.52-1.33 (m, 2H).

### Example 64. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-(4-(2-(2,6-dioxopiperidin-3-yl)-1,3-dioxoisoindolin-4-yl)piperazin-1-yl)butanoyl)piperidin-4-yl)-3-methoxybenzamide (Compound 64)

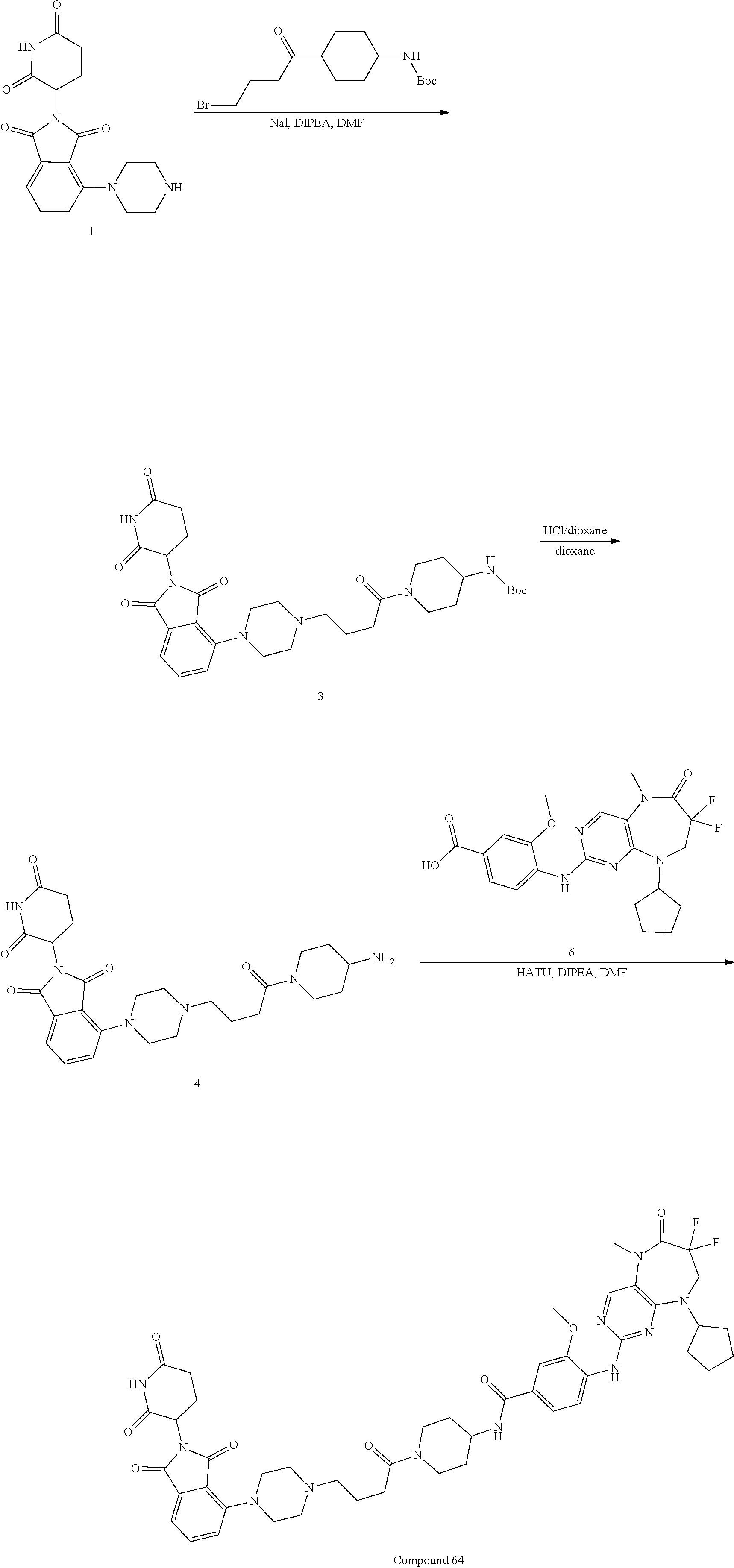 [SMILES: O=C1CCC(N2C(=O)c3cccc(N4CCNCC4)c3C2=O)C(=O)N1 ; CC(C)(C)OC(=O)NC1CCC(C(=O)CCCBr)CC1 ; CC(C)(C)OC(=O)NC1CCN(C(=O)CCCN2CCN(c3cccc4c3C(=O)N(C3CCC(=O)NC3=O)C4=O)CC2)CC1 ; NC1CCN(C(=O)CCCN2CCN(c3cccc4c3C(=O)N(C3CCC(=O)NC3=O)C4=O)CC2)CC1 ; COc1cc(C(=O)O)ccc1Nc1ncc2c(n1)N(C1CCCC1)CC(F)(F)C(=O)N2C ; COc1cc(C(=O)NC2CCN(C(=O)CCCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C]

The compound 64 was synthesized by the method described in the scheme similar to the method described in Example 60.

MS(M+H)⁺=940.1, ¹H NMR (400 MHz, DMSO-d₆) δ=11.08 (s, 1H), 8.31-8.23 (m, 2H), 8.15 (d, J=7.6 Hz, 1H), 7.96 (s, 1H), 7.69 (t, J=7.8 Hz, 1H), 7.51-7.46 (m, 2H), 7.34 (t, J=7.6 Hz, 2H), 5.09 (dd, J=5.3, 12.8 Hz, 1H), 4.82-4.71 (m, 1H), 4.40 (d, J=12.2 Hz, 1H), 4.04 (t, J=14.0 Hz, 3H), 3.93 (s, 4H), 3.34-3.30 (m, 7H), 3.12 (t, J=11.9 Hz, 1H), 2.93-2.81 (m, 1H), 2.71-2.61 (m, 2H), 2.57-2.52 (m, 4H), 2.41-2.30 (m, 4H), 2.07-1.77 (m, 6H), 1.76-1.67 (m, 4H), 1.65-1.54 (m, 4H), 1.50-1.33 (m, 2H).

### Example 65. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(5-(4-(2-(2,6-dioxopiperidin-3-yl)-1,3-dioxoisoindolin-4-yl)piperazin-1-yl)pentanoyl)piperidin-4-yl)-3-methoxybenzamide (Compound 65)

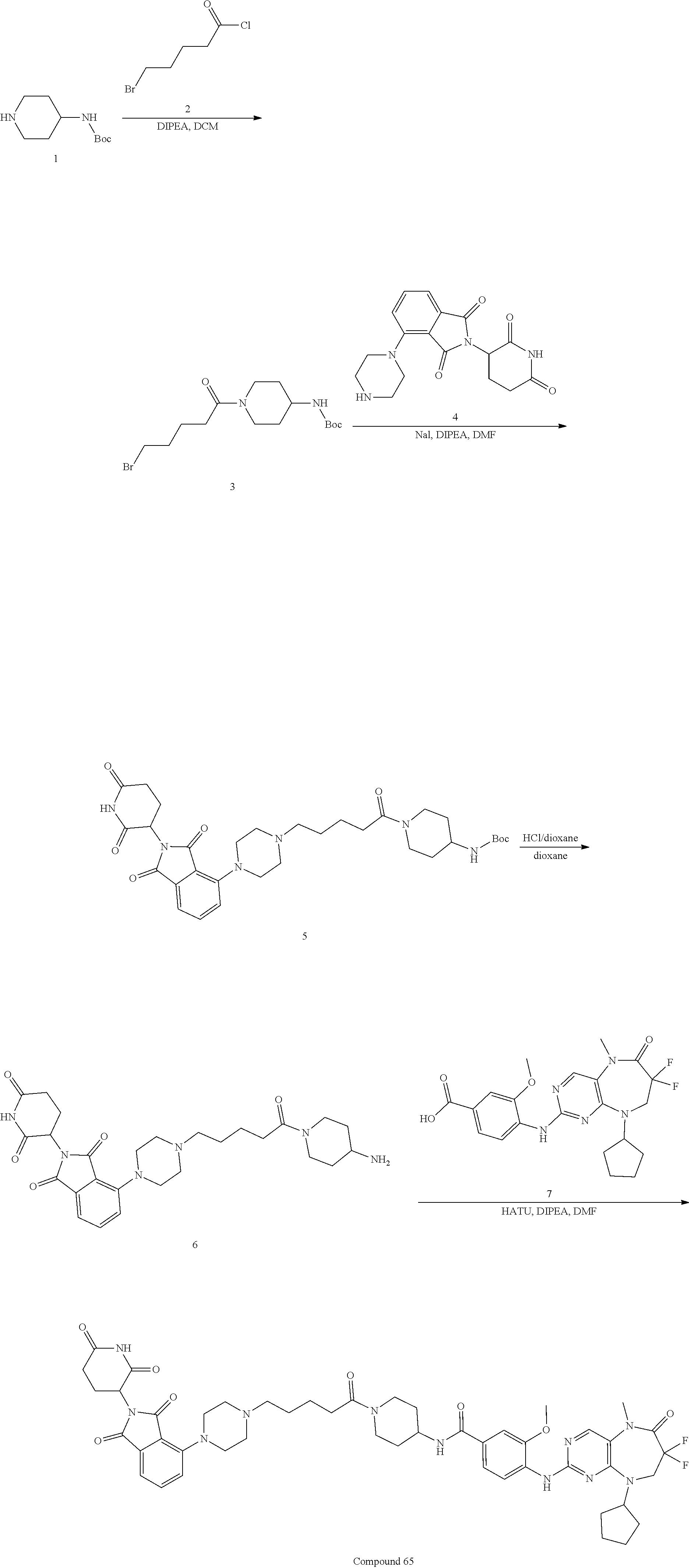

The compound 65 was synthesized by the method described in the scheme similar to the method described in Example 60.

MS(M+H)⁺=954.1, ¹H NMR (400 MHz, DMSO-d₆) δ=11.08 (br s, 1H), 8.29-8.25 (m, 2H), 8.17-8.15 (m, 1H), 7.96 (s, 1H), 7.70-7.67 (m, 1H), 7.50-7.46 (m, 2H), 7.36-7.31 (m, 2H), 5.11-5.07 (m, 1H), 4.81-4.71 (m, 1H), 4.45-4.36 (m, 1H), 4.09-3.98 (m, 3H), 3.93 (s, 3H), 3.32 (s, 3H), 3.31-3.28 (m, 4H), 3.15-3.05 (m, 1H), 2.92-2.81 (m, 1H), 2.72-2.59 (m, 2H), 2.55-2.51 (m, 4H), 2.37-2.33 (m, 4H), 2.06-1.98 (m, 1H), 1.97-1.84 (m, 3H), 1.81-1.80 (m, 1H), 176-1.32 (m, 13H).

### Example 66. Synthesis of 4 4-((7,7-difluoro-9-isopropyl-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (Compound 66)

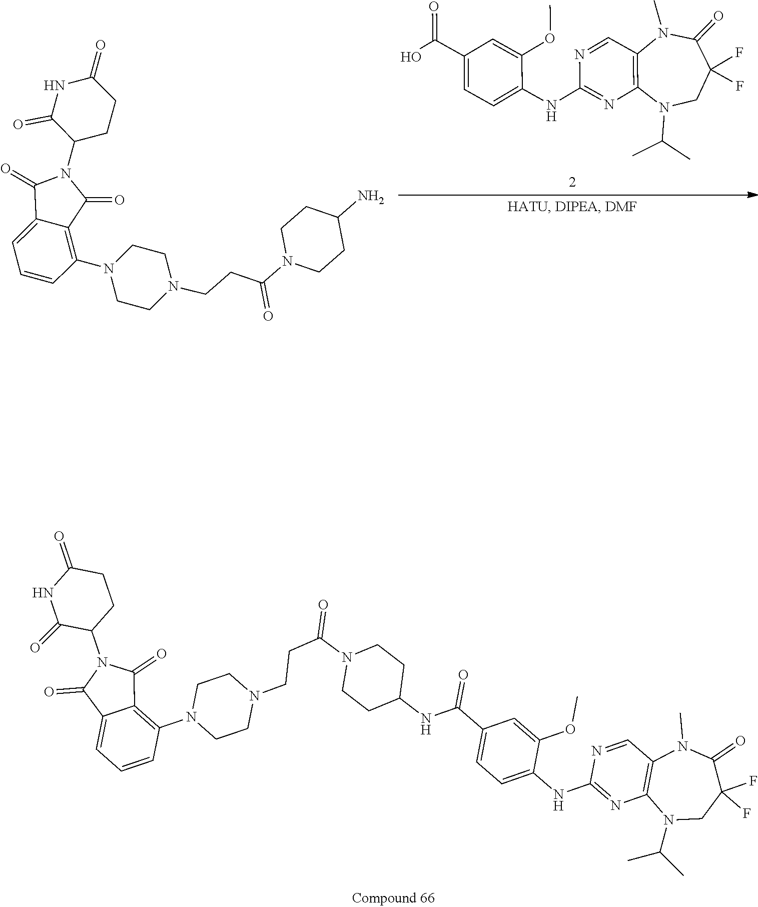 [SMILES: NC1CCN(C(=O)CCN2CCN(c3cccc4c3C(=O)N(C3CCC(=O)NC3=O)C4=O)CC2)CC1 ; COc1cc(C(=O)O)ccc1Nc1ncc2c(n1)N(C(C)C)CC(F)(F)C(=O)N2C ; COc1cc(C(=O)NC2CCN(C(=O)CCN3CCN(c4cccc5c4C(=O)N(C4CCC(=O)NC4=O)C5=O)CC3)CC2)ccc1Nc1ncc2c(n1)N(C(C)C)CC(F)(F)C(=O)N2C]

The compound 66 was synthesized by the method described in the scheme similar to the method described in Example 60.

MS(M+H)⁺=900.4, ¹H NMR (400 MHz, DMSO-d₆) δ=11.25-10.90 (m, 1H), 8.31 (d, J=8.3 Hz, 1H), 8.22 (s, 1H), 8.15 (br d, J=7.8 Hz, 1H), 7.88 (s, 1H), 7.70 (dd, J=7.4, 8.3 Hz, 1H), 7.56-7.46 (m, 2H), 7.41-7.30 (m, 2H), 5.09 (dd, J=5.4, 12.8 Hz, 1H), 4.88 (td, J=6.6, 13.4 Hz, 1H), 4.39 (br d, J=12.4 Hz, 1H), 4.12-3.95 (m, 4H), 3.93 (s, 3H), 3.30 (br s, 6H), 3.20-3.07 (m, 1H), 2.93-2.82 (m, 1H), 2.80-2.54 (m, 12H), 2.11-1.97 (m, 1H), 1.94-1.76 (m, 2H), 1.59-1.33 (m, 2H), 1.24 (d, J=6.8 Hz, 6H).

### Example 67. Synthesis of 4-((9-cyclopropyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (Compound 67)

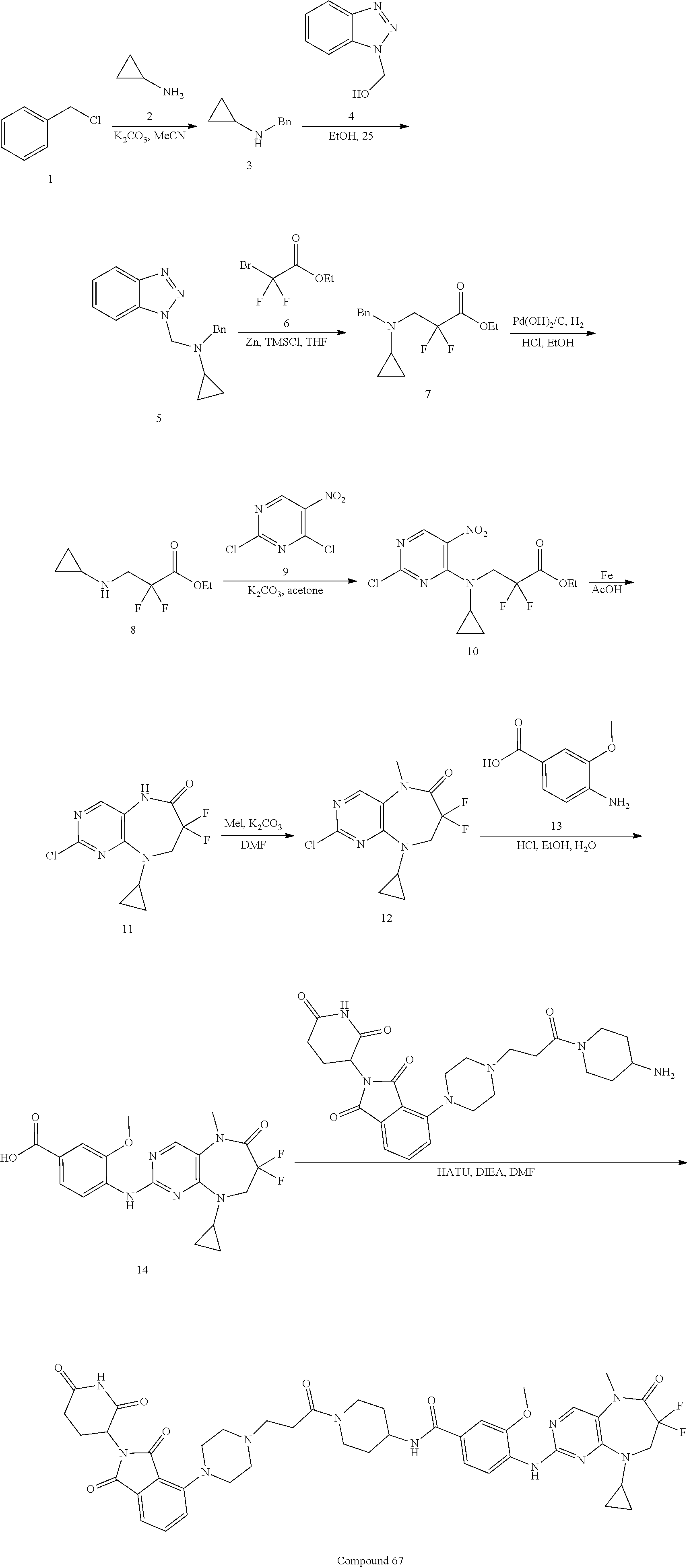

### Step 1. Synthesis of N-benzylcyclopropanamine (3)

To a solution of (chloromethyl)benzene (10 g, 79.00 mmol, 9.09 mL) and cyclopropanamine (11.28 g, 197.50 mmol, 13.68 mL) in ACN (100 mL) was added K₂CO₃ (16.38 g, 118.50 mmol), the mixture was stirred at 80° C. for 4 hours. TLC (Petroleum ether:EtOAc=10:1) indicated (chloromethyl)benzene was consumed completely and two new spots with larger polarity were detected. The mixture was filtered and the filter cake was washed with ACN (100 mL), the filtrate was concentrated in vacuum. The residue was diluted with toluene (30 mL) and concentrated in vacuum to remove most of cyclopropanamine to afford N-benzylcyclopropanamine (11.35 g) as yellow oil, which was used for the next step directly. MS(M+H)⁺=148.2

### Step 2. Synthesis of N-((1H-benzo[d][1,2,3]triazol-1-yl)methyl)-N-benzylcyclopropanamine (5)

A mixture of N-benzylcyclopropanamine (11.35 g, 77.10 mmol) and (1H-benzo[d][1,2,3]triazol-1-yl)methanol (11.50 g, 77.10 mmol) in EtOH (100 mL) was stirred at 25° C. for 16 hours. TLC (Petroleum ether:EtOAc=3:1) indicated N-benzylcyclopropanamine was consumed completely and one major new spot with lower polarity was detected. The reaction mixture was concentrated in vacuum to remove most of the solvent. The residue was diluted with H₂O (100 mL) and EtOAc (100 mL), the organic phase was separated, the aqueous phase was extracted with EtOAc (100 mL×1), the combined organic layers were dried over Na₂SO₄, filtered and concentrated in vacuum to afford N-((1H-benzo[d][1,2,3]triazol-1-yl)methyl)-N-benzylcyclopropanamine (20.9 g, crude) as light yellow oil, which was used directly. MS(M+H)⁺=279.4

### Step 3. Synthesis of ethyl 3-(benzyl(cyclopropyl)amino)-2,2-difluoropropanoate (7)

To a suspension of Zn (12.27 g, 187.71 mmol) in THF (100 mL) was added TMSCl (20.39 g, 187.71 mmol, 23.82 mL) dropwise at 0° C. under N₂. During which the temperature was maintained below 5° C. After stirring 20 minutes, ethyl 2-bromo-2,2-difluoroacetate (30.48 g, 150.17 mmol, 19.29 mL) was added slowly at 0° C. under N₂. During which the temperature was maintained below 30° C. The mixture was stirred for 20 minutes and cooled to 0° C. N-((1H-benzo[d][1,2,3]triazol-1-yl)methyl)-N-benzylcyclopropanamine (20.9 g, 75.09 mmol) in THF (100 mL) was added at 0° C. During which the temperature was maintained below 10° C. After 20 minutes the suspension was warmed to 15° C. and stirred for 5 hours. LCMS showed the reactant 1 was consumed completely and 55% of desired mass was detected. The mixture was filtered and the filter cake was washed with EtOAc (200 mL) and THF (200 mL), the filtrate was concentrated in vacuum. The residue was filtered and the filter cake was washed with the solvent (200 mL, Petroleum ether/EtOAc=6/1), the filtrate was concentrated in vacuum to afford ethyl 3-(benzyl(cyclopropyl)amino)-2,2-difluoropropanoate (54.5 g, crude) as a light yellow oil. MS(M+H)⁺=284.1

### Step 4. Synthesis of ethyl 3-(cyclopropylamino)-2,2-difluoropropanoate (8)

To a solution of ethyl 3-(benzyl(cyclopropyl)amino)-2,2-difluoropropanoate (54.5 g, 192.37 mmol) and HCl (12 M, 32.06 mL) in EtOH (200 mL) was added Pd(OH)₂/C (10 g, 10% purity) under N₂ atmosphere, the suspension was stirred at 25° C. under H₂ (15 psi) for 16 hours. LCMS showed 48% of the starting material remained, the mixture was stirred at 25° C. under H₂ (15 psi) for further 24 hours, LCMS showed trace of the starting material remained and desired mass was detected. The reaction mixture was filtered and the filter cake was washed with EtOH (500 mL), the filtrate was concentrated in vacuum to afford ethyl 3-(cyclopropylamino)-2,2-difluoropropanoate (45.4 g, HCl) as a red gum, which was used for the next step directly. MS(M+H)⁺=194.2

### Step 5. Synthesis of ethyl 3-((2-chloro-5-nitropyrimidin-4-yl)(cyclopropyl)amino)-2,2-difluoropropanoate (10)

To a solution of ethyl 3-(cyclopropylamino)-2,2-difluoropropanoate (45.4 g, 197.69 mmol, HCl) and 2,4-dichloro-5-nitropyrimidine (19.17 g, 98.85 mmol) in acetone (400 mL) was added K₂CO₃ (81.97 g, 593.07 mmol) at 0° C., during which the temperature was maintained below 10° C. The suspension was stirred at 25° C. for 16 hours. LCMS showed ethyl 3-(cyclopropylamino)-2,2-difluoropropanoate was consumed completely and 34% of desired mass was detected. After the reaction mixture was static and stratified, the supernatant was filtered and the filter cake was washed with EtOAc (500 mL), the sediment was diluted with H₂O (500 mL) and extracted with EtOAc (500 mL×2). The combined organic phase was concentrated in vacuum. The residue was purified by flash silica gel chromatography (80 g SepaFlash® Silica Flash Column, Eluent of 2˜12% EtOAc/Petroleum ether gradient @100 mL/min), the eluent was concentrated in vacuum. The residue was triturated with (Petroleum ether:EtOAc=15 1, 20 mL), the suspension was filtered and the filter cake was washed with (20 mL, Petroleum ether:EtOAc=15:1), the filter cake collected and dried to afford (ethyl 3-((2-chloro-5-nitropyrimidin-4-yl)(cyclopropyl)amino)-2,2-difluoropropanoate (8.95 g, 25.52 mmol, 12.91% yield)) as a white solid. The filtrate was concentrated in vacuum to afford the Product 2 (ethyl 3-((2-chloro-5-nitropyrimidin-4-yl)(cyclopropyl)amino)-2,2-difluoropropanoate (1 g) as a yellow gum. MS(M+H)⁺=350.7

### Step 6. Synthesis of 2-chloro-9-cyclopropyl-7,7-difluoro-8,9-dihydro-5H-pyrimido[4,5-b][1,4]diazepin-6(7H)-one (11)

A mixture of ethyl 3-((2-chloro-5-nitropyrimidin-4-yl)(cyclopropyl)amino)-2,2-difluoropropanoate (8.95 g, 25.52 mmol) and Fe (5.70 g, 102.08 mmol) in AcOH (80 mL) was stirred at 60° C. for 16 hours. LCMS showed the starting material was consumed completely and 97% of desired mass was detected. The mixture was concentrated in vacuo to remove most of the solvent. The residue was diluted with H₂O (300 mL) and EtOAc (200 mL), the suspension was filtered and the filter cake was washed with EtOAc (100 mL), the filtrate was stratified and the organic was collected, the aqueous phase was extracted with EtOAc (200 mL×2), the combined organic layers were washed with NaHCO₃ solution (100 mL×2), dried over Na₂SO₄, filtered and concentrated in vacuum. The residue was triturated with MTBE (20 mL) for 10 minutes, the suspension was filtered and the filter cake was washed with MTBE (10 mL), the filter cake was collected and dried to afford 2-chloro-9-cyclopropyl-7,7-difluoro-8,9-dihydro-5H-pyrimido[4,5-b][1,4]diazepin-6(7H)-one (6.2 g, 22.57 mmol, 88.44% yield) as a off-white solid, which was confirmed by H NMR. MS(M+H)⁺=275.0. The filtrate was concentrated in vacuum to afford 2-chloro-9-cyclopropyl-7,7-difluoro-8,9-dihydro-5H-pyrimido[4,5-b][1,4]diazepin-6(7H)-one (0.6 g) as a brown solid. MS(M+H)⁺=275.7

### Step 7. Synthesis of 2-chloro-9-cyclopropyl-7,7-difluoro-5-methyl-8,9-dihydro-5H-pyrimido[4,5-b][1,4]diazepin-6(7H)-one (12)

To a solution of 2-chloro-9-cyclopropyl-7,7-difluoro-8,9-dihydro-5H-pyrimido[4,5-b][1,4]diazepin-6(7H)-one (6.2 g, 22.57 mmol) in DMF (30 mL) were added K₂CO₃ (6.24 g, 45.15 mmol) and MeI (4.17 g, 29.35 mmol, 1.83 mL), the suspension was stirred at 25° C. for 3 hours. LCMS showed the starting material was consumed completely and 93% of desired mass was detected. The reaction mixture was quenched by addition of H₂O (30 mL) at 0° C., then diluted with ice water (300 mL) and extracted with EtOAc (150 mL×3). The combined organic layers were washed with brine (300 mL×5), dried over Na₂SO₄, filtered and concentrated in vacuo. The residue was purified by flash silica gel chromatography (20 g SepaFlash® Silica Flash Column, Eluent of 9˜40% EtOAc/Petroleum ether gradient @100 mL/min) to afford 2-chloro-9-cyclopropyl-7,7-difluoro-5-methyl-8,9-dihydro-5H-pyrimido[4,5-b][1,4]diazepin-6(7H)-one (6.12 g, 21.20 mmol, 93.91% yield) as a white solid. MS(M+H)⁺=288.7

### Step 8. Synthesis of 4-((9-cyclopropyl-7,7-difluoro-5-methyl-6-oxo-6,7,8,9-tetrahydro-5H-pyrimido[4,5-b][1,4]diazepin-2-yl)amino)-3-methoxybenzoic acid (14)

To a solution of 2-chloro-9-cyclopropyl-7,7-difluoro-5-methyl-8,9-dihydro-5H-pyrimido[4,5-b][1,4]diazepin-6(7H)-one (0.5 g, 1.73 mmol) and 4-amino-3-methoxybenzoic acid (434.29 mg, 2.60 mmol) in EtOH (3 mL) and H₂O (12 mL) was added HCl (12 M, 305.99 μL), the mixture was stirred at 100° C. for 16 hours. LCMS showed the reactant 1 was consumed completely and 77% of desired mass was detected. The reaction was concentrated in vacuo. The residue was triturated with H₂O (10 mL) for 5 minutes, the suspension was filtered and the filter cake was washed with H₂O (10 mL), the filter cake was collected and dried to give the crude product. The crude product was triturated with MTBE (10 mL) for 5 minutes, the suspension was filtered and the filter cake was washed with MTBE (10 mL), the filter cake was collected and dried to afford 4-((9-cyclopropyl-7,7-difluoro-5-methyl-6-oxo-6,7,8,9-tetrahydro-5H-pyrimido[4,5-b][1,4]diazepin-2-yl)amino)-3-methoxybenzoic acid (237 mg, 565.12 μmol, 32.63% yield) as a gray solid, which was confirmed by H NMR. MS(M+H)⁺=420.0

The filtrate was concentrated in vacuum to afford 4-((9-cyclopropyl-7,7-difluoro-5-methyl-6-oxo-6,7,8,9-tetrahydro-5H-pyrimido[4,5-b][1,4]diazepin-2-yl)amino)-3-methoxybenzoic acid (0.5 g) as a gray solid.

¹H NMR (400 MHz, DMSO-d₆) δ=8.76 (s, 1H), 8.63-8.51 (m, 1H), 8.40 (s, 1H), 7.64 (dd, J=1.3, 8.6 Hz, 1H), 7.54 (s, 1H), 4.30 (t, J=14.3 Hz, 2H), 3.95 (s, 3H), 3.30 (s, 3H), 3.02-2.97 (m, 1H), 0.95-0.81 (m, 2H), 0.78-0.60 (m, 2H).

### Step 9. Synthesis of 4-((9-cyclopropyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (Compound 67)

To a solution of 4-((9-cyclopropyl-7,7-difluoro-5-methyl-6-oxo-6,7,8,9-tetrahydro-5H-pyrimido[4,5-b][1,4]diazepin-2-yl)amino)-3-methoxybenzoic acid (70 mg, 166.91 μmol) in DMF (1 mL) were added HATU (82.50 mg, 216.99 μmol) and DIEA (86.29 mg, 667.65 μmol, 116.29 μL), the mixture was stirred at 25° C. for 15 minutes. To the mixture was added 4-(4-(3-(4-aminopiperidin-1-yl)-3-oxopropyl)piperazin-1-yl)-2-(2,6-dioxopiperidin-3-yl)isoindoline-1,3-dione (80.07 mg, 150.22 μmol, HCl), the mixture was stirred at 25° C. for 1 hour. LCMS showed 4-((9-cyclopropyl-7,7-difluoro-5-methyl-6-oxo-6,7,8,9-tetrahydro-5H-pyrimido[4,5-b][1,4]diazepin-2-yl)amino)-3-methoxybenzoic acid was consumed completely and 91% of desired mass was detected. To the mixture was added CH₃COOH to adjust pH<7. The mixture was purified by prep-HPLC (column: Waters Xbridge 150*25 mm*5 μm; mobile phase: [water(NH₄HCO₃)-ACN]; B %: 39%-69%, 9 min) followed by prep-HPLC (column: Unisil 3-100 C₁₈ μLtra 150*50 mm*3 μm; mobile phase: [water(FA)-ACN]; B %: 13%-43%, 10 min) and (column: Waters Xbridge 150*25 mm*5 μm; mobile phase: [water(NH₄HCO₃)-ACN]; B %: 33%-63%, 9 min), the eluent was freeze-dried to afford 4-((9-cyclopropyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (19.0 mg, 20.95 μmol, 12.55% yield, 99% purity) as a yellow solid. MS(M+H)⁺=898.2

¹H NMR (400 MHz, DMSO-d₆) δ=11.25-10.93 (m, 1H), 8.58 (d, J=8.4 Hz, 1H), 8.35 (s, 1H), 8.14 (d, J=7.7 Hz, 1H), 8.03 (s, 1H), 7.70 (t, J=7.8 Hz, 1H), 7.60-7.45 (m, 2H), 7.35 (t, J=7.3 Hz, 2H), 5.19-5.00 (m, 1H), 4.39 (d, J=12.8 Hz, 1H), 4.20 (t, J=15.5 Hz, 2H), 4.11-3.88 (m, 5H), 3.32-3.24 (m, 7H), 3.20-3.07 (m, 1H), 2.94-2.80 (m, 2H), 2.73-2.54 (m, 11H), 2.09-1.97 (m, 1H), 1.95-1.74 (m, 2H), 1.61-1.30 (m, 2H), 0.83 (br d, J=5.9 Hz, 2H), 0.72-0.55 (m, 2H).

### Example 68. Synthesis of 4-((9-cyclobutyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (Compound 68)

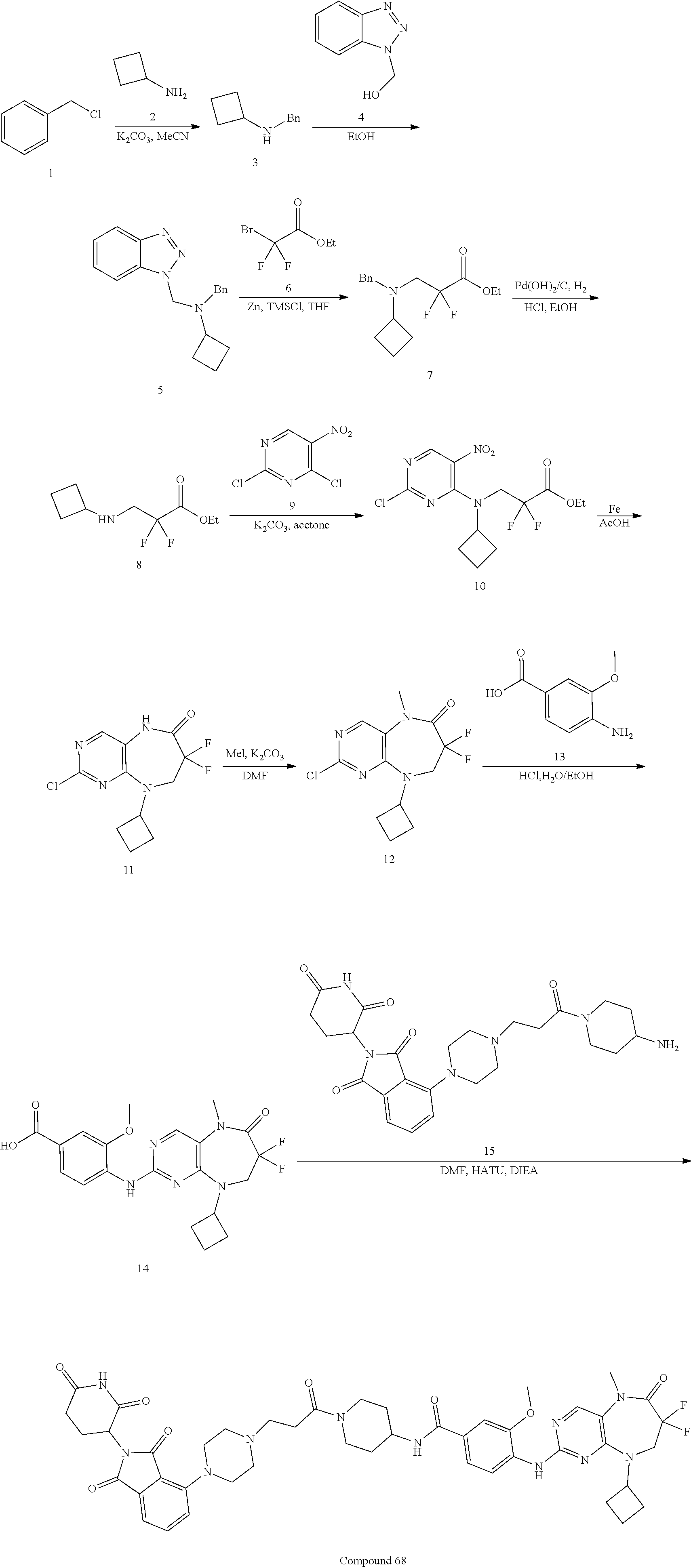

### Step 1. Synthesis of N-benzylcyclobutanamine (3)

To a solution of (chloromethyl)benzene (5 g, 39.50 mmol, 4.55 mL) in MeCN (50 mL) were added K₂CO₃ (16.38 g, 118.50 mmol) and cyclobutanamine (5.06 g, 71.10 mmol, 6.09 mL) at 25° C. The mixture was stirred at 80° C. for 5 h. TLC (petroleum ether:EtOAc=5:1) indicated one new spot was formed. The mixture was filtered. The filtrate was concentrated under reduced pressure. The residue was dissolved in toluene (30 ml) and then concentrated under reduced pressure to afford N-benzylcyclobutanamine (7.1 g, crude) as yellow oil. MS(M+H)⁺=162.3

¹H NMR (400 MHz, DMSO-d₆) δ=7.32-7.27 (m, 5H), 3.57 (s, 2H), 3.11-3.07 (m, 1H), 2.07-2.01 (m, 2H), 1.89-1.77 (m, 1H), 1.71-1.62 (m, 2H), 1.60-1.53 (m, 1H), 1.52-1.46 (m, 1H).

### Step 2. Synthesis of N-((1H-benzo[d][1,2,3]triazol-1-yl)methyl)-N-benzylcyclobutanamine (5)

To a solution of N-benzylcyclobutanamine (7.1 g, 44.03 mmol) in EtOH (80 mL) was added (1H-benzo[d][1,2,3]triazol-1-yl)methanol (6.57 g, 44.03 mmol) at 25° C. The mixture was stirred at 25° C. for 12 h. TLC (petroleum ether:EtOAc=1:1) indicated one new spot was formed. The reaction mixture was concentrated under reduced pressure. The residue was diluted with water (50 mL) and extracted with EtOAc (50 mL×3). The combined organic layers were washed with saturated brine (50 mL), dried over anhydrous sodium sulfate, filtered and concentrated under reduced pressure to afford N-((1H-benzo[d][1,2,3]triazol-1-yl)methyl)-N-benzylcyclobutanamine (13 g, crude) as yellow oil. MS(M+H)⁺=293.4

¹H NMR (400 MHz, DMSO-d₆) δ=8.05 (d, J=8.0 Hz, 1H), 7.69-7.67 (m, 1H), 7.56-7.52 (m, 1H), 7.46-7.39 (m, 1H), 7.37-7.35 (m, 5H), 5.44 (s, 2H), 3.63 (s, 2H), 3.30-3.26 (m, 1H), 1.94-1.90 (m, 2H), 1.87-1.82 (m, 2H), 1.59-1.50 (m, 2H).

### Step 3. Synthesis of ethyl 3-(benzyl(cyclobutyl)amino)-2,2-difluoropropanoate (7)

To a mixture of Zn (7.27 g, 111.16 mmol) in THF (100 ml) was added TMSCl (12.08 g, 111.16 mmol, 14.11 mL) at 0° C. After stirring for 30 minutes, ethyl 2-bromo-2,2-difluoroacetate (18.05 g, 88.93 mmol, 11.42 mL) was added to the mixture at 0° C., during which the temperature was maintained below 30° C. The mixture was stirred for 30 minutes and cooled to 0° C., then N-((1H-benzo[d][1,2,3]triazol-1-yl)methyl)-N-benzylcyclobutanamine (13 g, 44.46 mmol) in THF (100 ml) was added to at 0° C. and the resulting mixture was stirred at 25° C. for 4 h under N₂. LCMS showed ˜50% of desired mass was detected. The mixture was filtered and the filter cake was washed with EtOAc (200 mL) and THF (200 mL), the filtrate was washed with saturated sodium bicarbonate solution (200 mL) and concentrated in vacuo. The residue was purified by flash silica gel chromatography (ISCO®; 40 g SepaFlash® Silica Flash Column, Eluent of 0˜20% petroleum ether:EtOAc gradient @80 mL/min) to afford ethyl 3-(benzyl(cyclobutyl)amino)-2,2-difluoropropanoate (9.6 g, 21.63 mmol, 48.65% yield, 67% purity) as yellow oil. MS(M+H)⁺=298.1

### Step 4. Synthesis of ethyl 3-(cyclobutylamino)-2,2-difluoropropanoate (8)

To a mixture of ethyl 3-(benzyl(cyclobutyl)amino)-2,2-difluoropropanoate (9.6 g, 32.29 mmol) in EtOH (50 mL) were added Pd(OH)₂/C (2 g, 10% purity) and HCl (12 M, 5.38 mL) at 25° C. under nitrogen atmosphere. The mixture was stirred at 25° C. for 14 h under H₂ atmosphere (15 psi). TLC (petroleum ether:EtOAc=5:1) indicated two new spots were formed. The mixture was filtered. The filtrate was concentrated under reduced pressure to afford ethyl 3-(cyclobutylamino)-2,2-difluoropropanoate (8 g, crude) as yellow oil. MS(M+H)⁺=208.2

¹H NMR (400 MHz, DMSO-d₆)=4.36-4.30 (q, J=7.2 Hz, 2H), 3.45-3.40 (q, J=7.2 Hz, 1H), 2.31-2.24 (m, 2H), 2.18-2.13 (m, 2H), 1.79-1.70 (m, 2H), 1.28 (t, J=7.2 Hz, 3H), 1.04 (t, J=6.8 Hz, 2H).

### Step 5. Synthesis of ethyl 3-((2-chloro-5-nitropyrimidin-4-yl)(cyclobutyl)amino)-2,2-difluoropropanoate (10)

To a solution of ethyl 3-(cyclobutylamino)-2,2-difluoropropanoate (8 g, 38.61 mmol) in acetone (50 mL) were added K₂CO₃ (10.67 g, 77.21 mmol) and 2,4-dichloro-5-nitropyrimidine (8.24 g, 42.47 mmol) at 25° C. The mixture was stirred at 25° C. for 12 h. LCMS showed ˜46% of desired mass was detected. The mixture was filtered. The filtrate was concentrated under reduced pressure. The residue was purified by flash silica gel chromatography (ISCO®; 40 g SepaFlash® Silica Flash Column, Eluent of 0˜10% petroleum ether:EtOAc ether gradient @80 mL/min) to afford ethyl 3-((2-chloro-5-nitropyrimidin-4-yl)(cyclobutyl)amino)-2,2-difluoropropanoate (3.9 g, 10.41 mmol, 26.98% yield, 97.4% purity) as yellow oil. MS(M+H)⁺=364.9

¹H NMR (400 MHz, DMSO-d₆)=9.05 (s, 1H), 4.59 (t, J=14.8 Hz, 2H), 4.28-4.22 (q, J=7.2 Hz, 2H), 4.04-3.98 (m, 1H), 2.02-1.98 (m, 4H), 1.62-1.52 (m, 2H), 1.20 (t, J=7.2 Hz, 3H).

### Step 6. Synthesis of 2-chloro-9-cyclobutyl-7,7-difluoro-8,9-dihydro-5H-pyrimido[4,5-b][1,4]diazepin-6(7H)-one (11)

To a solution of ethyl 3-((2-chloro-5-nitropyrimidin-4-yl)(cyclobutyl)amino)-2,2-difluoropropanoate (3.9 g, 10.69 mmol) in HOAc (20 mL) was added Fe (2.39 g, 42.77 mmol) at 25° C. The mixture was stirred at 80 C for 5 h. LCMS showed main peak with desired mass. The mixture was filtered. The filtrate was diluted with water (100 mL) and extracted with EtOAc (100 mL×3). The combined organic layers were washed with saturated sodium bicarbonate solution (200 ml) and brine (200 mL), dried over anhydrous sodium sulfate, filtered and concentrated under reduced pressure to afford 2-chloro-9-cyclobutyl-7,7-difluoro-8,9-dihydro-5H-pyrimido[4,5-b][1,4]diazepin-6(7H)-one (3 g, crude) as a brown solid. MS(M+H)⁺=289.0

### Step 7. Synthesis of 2-chloro-9-cyclobutyl-7,7-difluoro-5-methyl-8,9-dihydro-5H-pyrimido[4,5-b][1,4]diazepin-6(7H)-one (12)

To a solution of 2-chloro-9-cyclobutyl-7,7-difluoro-8,9-dihydro-5H-pyrimido[4,5-b][1,4]diazepin-6(7H)-one (3 g, 10.39 mmol) in DMF (30 mL) were added K₂CO₃ (4.31 g, 31.18 mmol) and MeI (2.95 g, 20.78 mmol, 1.29 mL) at 25° C. The mixture was stirred at 25° C. for 5 h. LCMS showed ˜60% of desired mass was detected. The reaction mixture was diluted with water (50 mL) and extracted with EtOAc (50 mL×3). The combined organic layers were washed with saturated brine (50 mL), dried over anhydrous sodium sulfate, filtered and concentrated under reduced pressure. The residue was purified by flash silica gel chromatography (ISCO®; 20 g SepaFlash® Silica Flash Column, eluent of 0-20% petroleum ether/EtOAc gradient @60 mL/min) to afford 2-chloro-9-cyclobutyl-7,7-difluoro-5-methyl-8,9-dihydro-5H-pyrimido[4,5-b][1,4]diazepin-6(7H)-one (2.3 g, 7.45 mmol, 71.65% yield, 98% purity) as a light yellow solid. MS(M+H)⁺=303.1

¹H NMR (400 MHz, DMSO-d₆) 8.41 (s, 1H), 4.45-4.36 (m, 1H), 4.18 (t, J=14.4 Hz, 2H), 3.35 (s, 3H), 2.21-2.20 (m, 2H), 2.10-2.07 (m, 2H), 1.70-1.66 (m, 2H).

### Step 8. Synthesis of 4-((9-cyclobutyl-7,7-difluoro-5-methyl-6-oxo-6,7,8,9-tetrahydro-5H-pyrimido[4,5-b][1,4]diazepin-2-yl)amino)-3-methoxybenzoic acid (14)

To a solution of 2-chloro-9-cyclobutyl-7,7-difluoro-5-methyl-8,9-dihydro-5H-pyrimido[4,5-b][1,4]diazepin-6(7H)-one (0.5 g, 1.65 mmol) in H₂O (6 mL) and EtOH (2 mL) were added HCl (12 M, 275.00 μL) and 4-amino-3-methoxybenzoic acid (330.98 mg, 1.98 mmol) at 25° C. The mixture was stirred at 100° C. for 12 h. LCMS showed ˜60% of desired mass. The residue was triturated by EtOAc/ethanol (v/v=1:1) (10 ml×3) to afford 4-((9-cyclobutyl-7,7-difluoro-5-methyl-6-oxo-6,7,8,9-tetrahydro-5H-pyrimido[4,5-b][1, 4]diazepin-2-yl)amino)-3-methoxybenzoic acid (400 mg, 756.79 μmol, 45.87% yield, 82% purity) as a gray solid. MS(M+H)⁺=434.1

### Step 9. Synthesis of 4-((9-cyclobutyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (Compound 68)

To a solution of 4-((9-cyclobutyl-7,7-difluoro-5-methyl-6-oxo-6,7,8,9-tetrahydro-5H-pyrimido[4,5-b][1, 4]diazepin-2-yl)amino)-3-methoxybenzoic acid (0.2 g, 461.46 μmol) in DMF (3 mL) were added HATU (350.92 mg, 922.92 μmol) and DIEA (178.92 mg, 1.38 mmol, 241.13 μL) at 25° C., after stirring for 0.5 h, then 4-(4-(3-(4-aminopiperidin-1-yl)-3-oxopropyl)piperazin-1-yl)-2-(2,6-dioxopiperidin-3-yl)isoindoline-1,3-dione (229.14 mg, 461.46 μmol, HCl salt) was added and the resulting mixture was stirred at 25° C. for 12 h. LCMS showed ˜55% of desired mass was detected. The reaction mixture was diluted with water (50 mL) and extracted with EtOAc (50 mL×5). The combined organic layers were washed with saturated brine (150 mL), dried over anhydrous sodium sulfate, filtered and concentrated under reduced pressure. The residue was purified by prep-HPLC (column: Phenomenex luna C18 150×40 mm×15 μm; mobile phase: [water(FA)-ACN]; B %: 17%-47%, 10 min) and further purified by prep-HPLC (column: Waters Xbridge 150×25 mm×5 μm; mobile phase: [water(NH₄HCO₃)-ACN]; B %: 38%-68%, 10 min) followed by lyophilization to afford 4-((9-cyclobutyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (64.3 mg, 65.93 μmol, 14.29% yield, 93.5% purity) as a yellow solid. MS(M+H)⁺=912.4

¹H NMR (400 MHz, DMSO-d₆) δ=11.08 (s, 1H), 8.35-8.32 (m, 2H), 8.17 (d, J=8 Hz, 1H), 7.99 (s, 1H), 7.71-7.67 (m, 1H), 7.53-7.51 (dd, J=8.4, 1.6 Hz, 1H), 7.49-7.48 (m, 1H), 7.36-7.32 (m, 2H), 5.10-5.05 (m, 1H), 4.40-4.36 (m, 2H), 4.07-3.97 (m, 4H), 3.92 (s, 3H), 3.33 (s, 3H), 3.31-3.27 (m, 4H), 3.16-3.13 (m, 1H), 2.91-2.83 (m, 1H), 2.59-2.55 (m, 11H), 2.23-2.20 (m, 2H), 2.06-2.00 (m, 3H), 1.90-1.80 (m, 2H), 1.71-1.65 (m, 2H), 1.50-1.38 (m, 2H).

### Example 69. Synthesis of 4-((7-allyl-9-cyclopentyl-5-methyl-6-oxo-6,7,8,9-tetrahydro-5H-pyrimido[4,5-b][1, 4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (Compound 69)

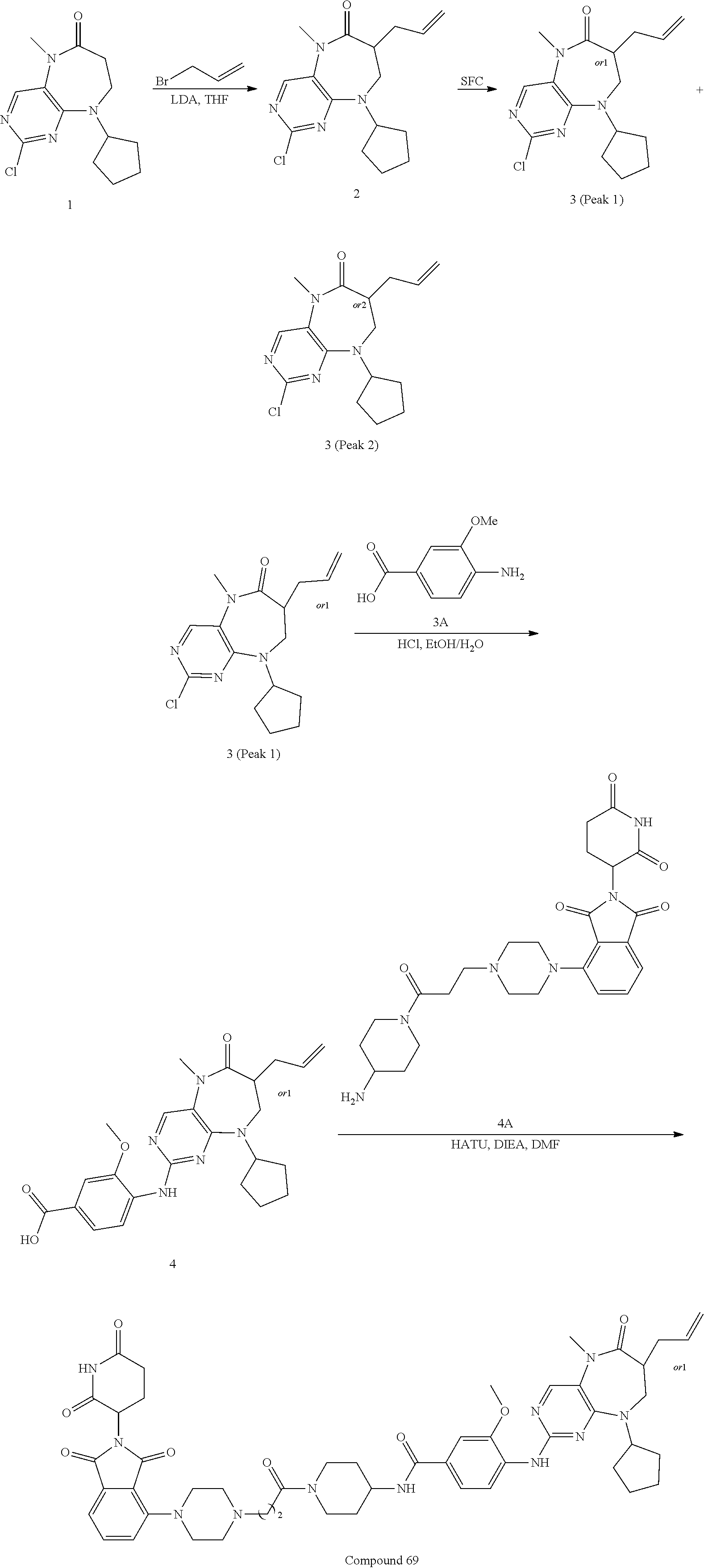

### Step 1. Synthesis of 7-allyl-2-chloro-9-cyclopentyl-5-methyl-8,9-dihydro-5H-pyrimido[4,5-b][1,4]diazepin-6(7H)-one (2)

To the solution of 2-chloro-9-cyclopentyl-5-methyl-8,9-dihydro-5H-pyrimido[4,5-b][1,4]diazepin-6(7H)-one (0.4 g, 1.42 mmol) in THF (8 mL) was added LDA (2 M, 854.85 μL) at −78° C., the mixture was stirred at −78° C. for 30 min, 3-bromoprop-1-ene (689.44 mg, 5.70 mmol, 4 eq) was added and the resulting mixture was stirred at −78° C. for 2 h and stirred at 20° C. for another 12 h. LCMS showed that the reaction was completed. The mixture was quenched with ammonium chloride (sat. aq., 50 mL) and water (20 mL), extracted with EtOAc (2×50 mL) and the combined organic extracts were washed with brine (50 mL), dried over Na₂SO₄, filtered and concentrated in vacuo. The residue was purified by flash silica gel chromatography (5 g SepaFlash® Silica Flash Column, Eluent of 0˜50% EtOAc/Petroleum ether gradient @80 mL/min) to afford 7-allyl-2-chloro-9-cyclopentyl-5-methyl-8,9-dihydro-5H-pyrimido[4,5-b][1,4]diazepin-6(7H)-one (0.3 g, 907.06 μmol, 63.66% yield, 97% purity) as yellow solid. MS(M+H)⁺=321.2

### Step 2. Synthesis of 7-allyl-2-chloro-9-cyclopentyl-5-methyl-8,9-dihydro-5H-pyrimido[4,5-b][1,4]diazepin-6(7H)-one(3 (Peak1)) and 7-allyl-2-chloro-9-cyclopentyl-5-methyl-8,9-dihydro-5H-pyrimido[4,5-b][1,4]diazepin-6(7H)-one(3 (Peak2))

7-allyl-2-chloro-9-cyclopentyl-5-methyl-8,9-dihydro-5H-pyrimido[4,5-b][1,4]diazepin-6(7H)-one (0.3 g, 0.935 mmol) was separated by SFC (column: DAICEL CHIRALCEL OJ (250 mm*30 mm, 10 m); mobile phase: [0.1% NH₃H₂O IPA]; B %: 25%-25%, 3.7 min; 30 min) to afford 7-allyl-2-chloro-9-cyclopentyl-5-methyl-8,9-dihydro-5H-pyrimido[4,5-b][1,4]diazepin-6(7H)-one (110 mg, 336.02 μmol, 35.93% yield, 98% purity) (Peak1) and 7-allyl-2-chloro-9-cyclopentyl-5-methyl-8,9-dihydro-5H-pyrimido[4,5-b][1,4]diazepin-6(7H)-one (120 mg, 370.31 μmol, 39.60% yield, 99% purity) (Peak2) as yellow oil. MS(M+H)⁺=321.0

### Step 3. Synthesis of 4-((7-allyl-9-cyclopentyl-5-methyl-6-oxo-6,7,8,9-tetrahydro-5H-pyrimido[4,5-b][1, 4]diazepin-2-yl)amino)-3-methoxybenzoic acid (4)

To the solution of 7-allyl-2-chloro-9-cyclopentyl-5-methyl-8,9-dihydro-5H-pyrimido[4,5-b][1,4]diazepin-6(7H)-one (Peak1, 100 mg, 311.70 μmol) and 4-amino-3-methoxy-benzoic acid (57.32 mg, 342.87 μmol) in H₂O (3 mL) and EtOH (1.5 mL) was added HCl (12 M, 57.15 μL) and the mixture was stirred at 90° C. for 12 h. LCMS showed that the reaction was completed. The mixture was filtered and filter cake was collected to afford 4-((7-allyl-9-cyclopentyl-5-methyl-6-oxo-6,7,8,9-tetrahydro-5H-pyrimido[4,5-b][1,4]diazepin-2-yl)amino)-3-methoxybenzoic acid (110 mg, 238.75 μmol, 76.60% yield, 98% purity) as white solid. MS(M+H)⁺=452.2

### Step 4. Synthesis of 4-((7-allyl-9-cyclopentyl-5-methyl-6-oxo-6,7,8,9-tetrahydro-5H-pyrimido[4,5-b][1, 4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (Compound 69)

To the solution of 4-((7-allyl-9-cyclopentyl-5-methyl-6-oxo-6,7,8,9-tetrahydro-5H-pyrimido[4,5-b][1,4]diazepin-2-yl)amino)-3-methoxybenzoic acid (100.00 mg, 221.48 μmol) and 4-(4-(3-(4-aminopiperidin-1-yl)-3-oxopropyl)piperazin-1-yl)-2-(2,6-dioxopiperidin-3-yl)isoindoline-1,3-dione (118.05 mg, 221.48 μmol, HCl) in DMF (3 mL) was added HATU (126.32 mg, 332.21 μmol) and DIPEA (85.87 mg, 664.43 μmol, 115.73 μL) and the resulting mixture was stirred at 20° C. for 12 h. LCMS showed that the reaction was completed. The reaction was adjusted pH=7 by FA, the resulting mixture was purified by prep-HPLC (column: Phenomenex Luna C₁₈ 150*25 mm*10 μm; mobile phase: [water(FA)-ACN]; B %: 25%-55%, 10 min) and the eluent was lyophilized to afford 4-((7-allyl-9-cyclopentyl-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl) piperazin-1-yl)propanoyl)piperidin-4-yl)-3-methoxybenzamide (97.3 mg, 102.52 μmol, 46.29% yield, 98% purity) as yellow solid. MS(M+H)⁺=930.4

¹H NMR (400 MHz, DMSO-d₆) δ=11.09 (s, 1H), 8.38 (d, J=8.9 Hz, 1H), 8.17-8.11 (m, 2H), 8.10 (s, 1H), 7.76 (s, 1H), 7.73-7.65 (m, 1H), 7.50-7.45 (m, 2H), 7.37-7.35 (m, 2H), 5.86-5.73 (m, 1H), 5.12-5.01 (m, 3H), 4.87-4.78 (m, 1H), 4.40 (d, J=12.9 Hz, 1H), 4.10-4.03 (m, 1H), 3.98-3.91 (m, 4H), 3.45-3.35 (m, 3H), 3.19 (s, 3H), 3.18-3.08 (m, 2H), 2.93-2.75 (m, 3H), 2.72-2.56 (m, 11H), 2.46-2.39 (m, 1H), 2.08-1.99 (m, 3H), 1.90-1.78 (m, 3H), 1.76-1.53 (m, 6H), 1.52-1.38 (m, 3H).

### Example 70. Synthesis of 4-((7-allyl-9-cyclopentyl-5-methyl-6-oxo-6,7,8,9-tetrahydro-5H-pyrimido[4,5-b][1, 4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (Compound 70)

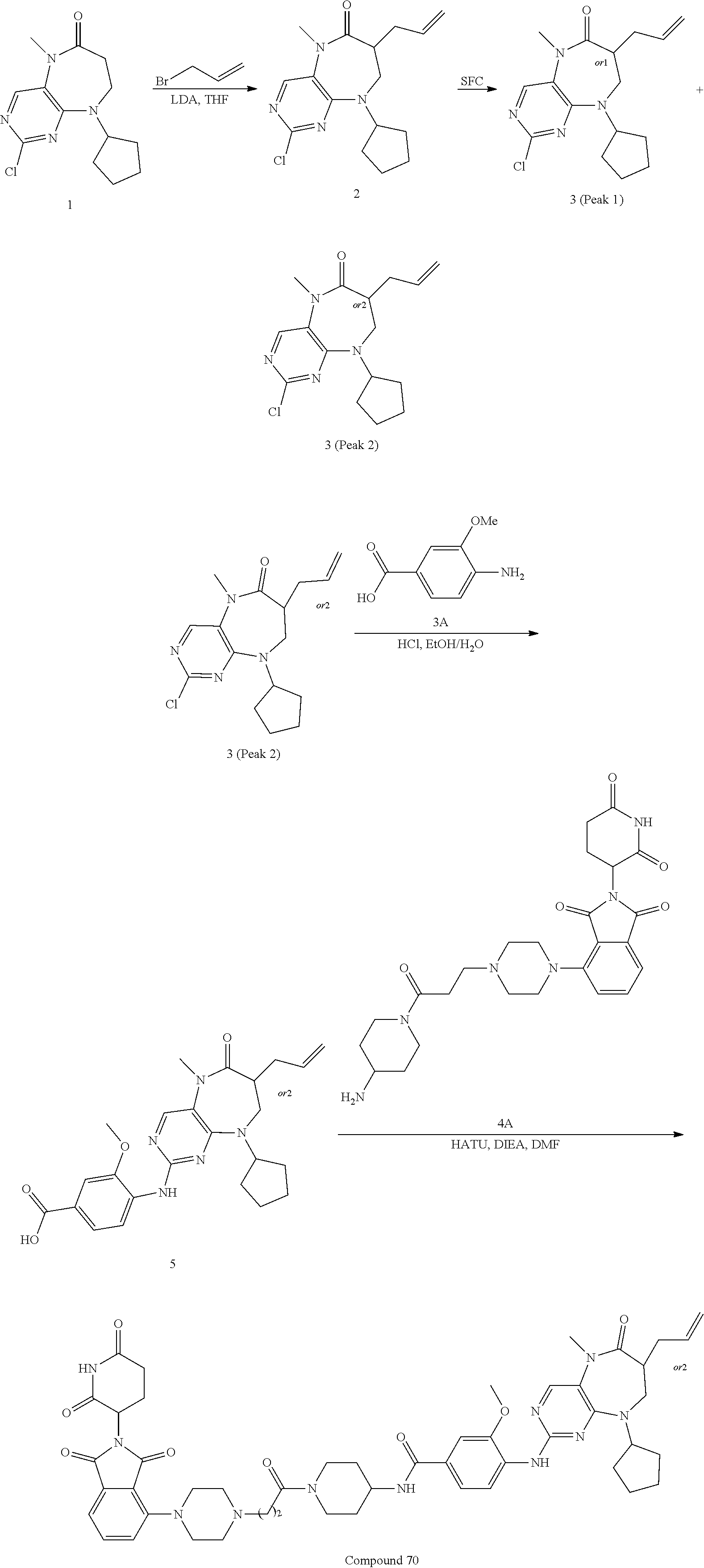

### Step 1. Synthesis of 4-((7-allyl-9-cyclopentyl-5-methyl-6-oxo-6,7,8,9-tetrahydro-5H-pyrimido[4,5-b][1,4]diazepin-2-yl)amino)-3-methoxybenzoic acid (5)

To the solution of 7-allyl-2-chloro-9-cyclopentyl-5-methyl-8,9-dihydro-5H-pyrimido[4,5-b][1,4]diazepin-6(7H)-one (Peak2, 120 mg, 374.05 μmol) and 4-amino-3-methoxy-benzoic acid (68.78 mg, 411.45 μmol) in H₂O (3 mL) and EtOH (1.5 mL) was added HCl (12 M, 68.58 μL) and the mixture was stirred at 90° C. for 12 h. LCMS showed that the reaction was completed. The mixture was filtered and filter cake was collected to afford 4-((7-allyl-9-cyclopentyl-5-methyl-6-oxo-6,7,8,9-tetrahydro-5H-pyrimido[4,5-b][1,4]diazepin-2-yl)amino)-3-methoxybenzoic acid (120 mg, 265.77 μmol, 71.05% yield, 100% purity) was obtained as white solid. MS(M+H)⁺=452.2

### Step 2. Synthesis of 4-((7-allyl-9-cyclopentyl-5-methyl-6-oxo-6,7,8,9-tetrahydro-5H-pyrimido[4,5-b][1, 4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (Compound 70)

To the solution of 4-((7-allyl-9-cyclopentyl-5-methyl-6-oxo-6,7,8,9-tetrahydro-5H-pyrimido[4,5-b][1,4]diazepin-2-yl)amino)-3-methoxybenzoic acid (120 mg, 265.77 μmol) and 4-(4-(3-(4-aminopiperidin-1-yl)-3-oxopropyl)piperazin-1-yl)-2-(2,6-dioxopiperidin-3-yl)isoindoline-1,3-dione (141.66 mg, 265.77 μmol, HCl) in DMF (3 mL) was added HATU (151.58 mg, 398.66 μmol) and DIPEA (103.04 mg, 797.31 μmol, 138.87 μL) and the resulting mixture was stirred at 20° C. for 12 h. LCMS showed that the reaction was completed. The reaction was adjusted pH=7 by FA, the resulting mixture was purified by prep-HPLC (column: Phenomenex Luna C₁₈ 150*25 mm*10 μm; mobile phase: [water(FA)-ACN]; B %: 25%-55%, 10 min) and the eluent was lyophilized to afford 4-((7-allyl-9-cyclopentyl-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl) piperazin-1-yl)propanoyl)piperidin-4-yl)-3-methoxybenzamide (144.3 mg, 140.44 μmol, 52.84% yield, 95% purity, FA) as yellow solid. MS(M+H)⁺=930.4

¹H NMR (400 MHz, DMSO-d₆) δ=11.11 (s, 1H), 8.39 (d, J=8.8 Hz, 1H), 8.20-8.12 (m, 2H), 8.11 (s, 1H), 7.78 (s, 1H), 7.73 (t, J=7.8 Hz, 1H), 7.50-7.47 (m, 2H), 7.41-7.36 (m, 2H), 5.85-5.76 (m, 1H), 5.13-5.02 (m, 3H), 4.86-4.82 (m, 1H), 4.43-4.39 (m, 1H), 4.07-4.03 (m, 1H), 3.95 (s, 4H), 3.42-3.41 (m, 3H), 3.20 (s, 3H), 3.17-3.12 (m, 2H), 2.94-2.83 (m, 3H), 2.82-2.56 (m, 11H), 2.46-2.40 (m, 1H), 2.09-2.00 (m, 3H), 1.92-1.76 (m, 4H), 1.71-1.57 (m, 5H), 1.52-1.40 (m, 3H).

### Example 71. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methylbenzamide (Compound 71)

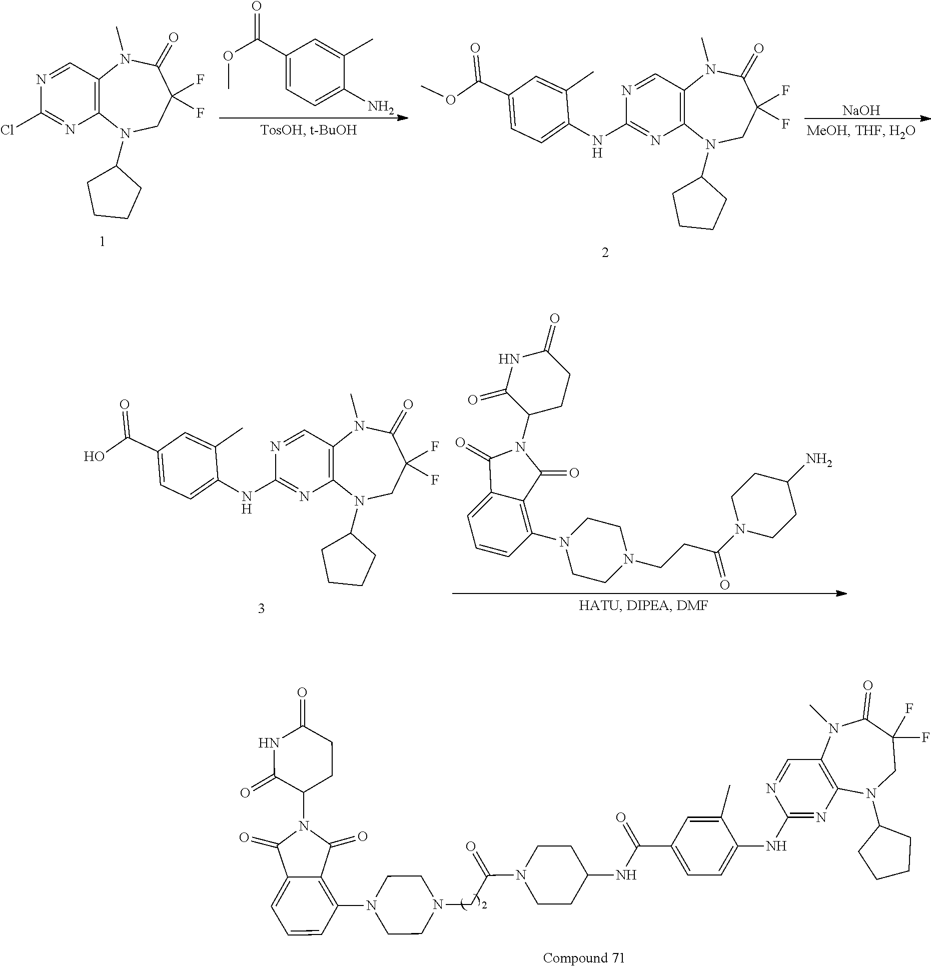

### Step 1. Synthesis of methyl 4-((9-cyclopentyl-7,7-difluoro-5-methyl-6-oxo-6,7,8,9-tetrahydro-5H-pyrimido[4,5-b][1,4]diazepin-2-yl)amino)-3-methylbenzoate (2)

To a solution of 2-chloro-9-cyclopentyl-7,7-difluoro-5-methyl-8,9-dihydro-5H-pyrimido[4,5-b][1,4]diazepin-6(7H)-one (100 mg, 315.72 μmol) and methyl 4-amino-3-methylbenzoate (62.58 mg, 378.87 μmol) in t-BuOH (2 mL) was added TsOH (163.10 mg, 947.17 μmol), the mixture was stirred at 100° C. for 16 hours. LCMS showed the starting material was consumed completely 68% of the mass of product was detected. The mixture was concentrated in vacuo to afford methyl 4-((9-cyclopentyl-7,7-difluoro-5-methyl-6-oxo-6,7,8,9-tetrahydro-5H-pyrimido[4,5-b][1,4]diazepin-2-yl)amino)-3-methylbenzoate (200 mg) as a brown oil, which was used for the next step directly. MS(M+H)⁺=446.2

### Step 2. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-3-methylbenzoic acid (3)

To a solution of methyl 4-((9-cyclopentyl-7,7-difluoro-5-methyl-6-oxo-6,7,8,9-tetrahydro-5H-pyrimido[4,5-b][1,4]diazepin-2-yl)amino)-3-methylbenzoate (200 mg, 448.97 μmol) in MeOH (2 mL), THF (2 mL) and H₂O (2 mL) was added NaOH (538.73 mg, 13.47 mmol), the mixture was stirred at 25° C. for 16 hours. LCMS showed the starting material was consumed completely 84% of desired mass was detected. The mixture was concentrated in vacuo to remove most of the solvent. To the residue was added HCl solution (12 M) to adjust pH<3 at 0° C. The suspension was filtered and the filter cake was collected and dried to afford 4-((9-cyclopentyl-7,7-difluoro-5-methyl-6-oxo-6,7,8,9-tetrahydro-5H-pyrimido[4,5-b][1,4]diazepin-2-yl)amino)-3-methylbenzoic acid (140 mg) as a brown solid, which was used for the next step directly. MS(M+H)⁺=432.2

### Step 3. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methylbenzamide (Compound 71)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methylbenzoic acid (65 mg, 150.66 μmol) in DMF (3 mL) were added HATU (68.74 mg, 180.79 μmol) and DIEA (116.83 mg, 903.96 μmol, 157.45 μL), the mixture was stirred at 15° C. for 15 minutes, to the mixture was added 4-(4-(3-(4-aminopiperidin-1-yl)-3-oxopropyl)piperazin-1-yl)-2-(2,6-dioxopiperidin-3-yl)isoindoline-1,3-dione (72.27 mg, 135.59 μmol, HCl salt), the resulting mixture was stirred at 15° C. for 1 hour. LCMS showed 4-((9-cyclopentyl-7,7-difluoro-5-methyl-6-oxo-6,7,8,9-tetrahydro-5H-pyrimido[4,5-b][1,4]diazepin-2-yl)amino)-3-methylbenzoic acid was consumed completely and 69% of desired mass was detected. To the mixture was added CH₃COOH to adjust pH<7. The resulting mixture was purified by prep-HPLC (column: Waters Xbridge 150*25 mm*5 μm; mobile phase: [water (10 mM NH₄HCO₃)-ACN]; B %: 34%-64%, min) followed by prep-HPLC (column: Phenomenex luna C₁₈ 150*25 mm*10 μm; mobile phase: [water (0.225% FA)-ACN]; B %: 14%-44%, 10 min), the eluent was freeze-dried to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methylbenzamide (25.0 mg, 26.92 μmol, 17.87% yield, 98% purity) as a yellow solid. MS(M+H)⁺=910.3

¹H NMR (400 MHz, DMSO-d₆) δ=11.31-10.77 (m, 1H), 8.59 (s, 1H), 8.19 (s, 1H), 8.12 (d, J=7.7 Hz, 1H), 7.75 (d, J=8.5 Hz, 1H), 7.73-7.67 (m, 2H), 7.64 (dd, J=1.9, 8.4 Hz, 1H), 7.40-7.29 (m, 2H), 5.08 (dd, J=5.3, 12.9 Hz, 1H), 4.60 (q, J=8.3 Hz, 1H), 4.37 (d, J=13.1 Hz, 1H), 4.07-3.89 (m, 4H), 3.32-3.28 (m, 7H), 3.13 (t, J=11.7 Hz, 1H), 2.92-2.81 (m, 1H), 2.67-2.52 (m, 11H), 2.29 (s, 3H), 2.07-1.97 (m, 1H), 1.90-1.76 (m, 4H), 1.65-1.60 (m, 2H), 1.58-1.43 (m, 5H), 1.43-1.37 (m, 1H).

### Example 72. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-hydroxybenzamide (Compound 72)

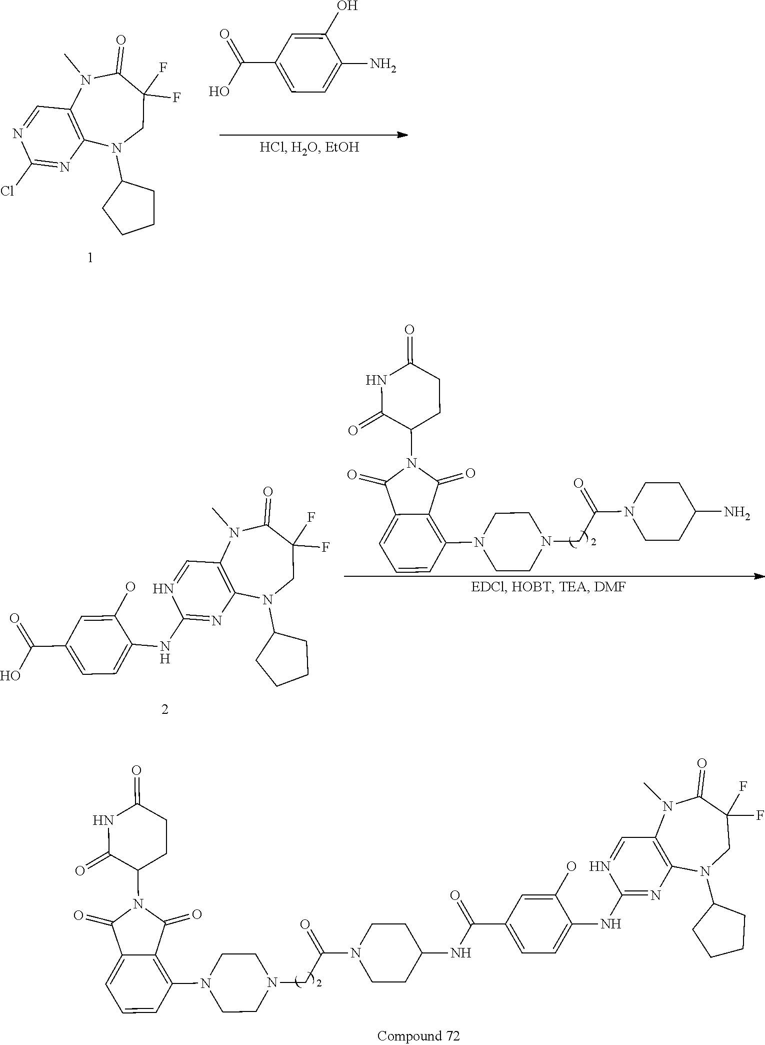

### Step 1. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-3-hydroxybenzoic acid (2)

To the solution of 2-chloro-9-cyclopentyl-7,7-difluoro-5-methyl-8,9-dihydro-5H-pyrimido[4,5-b][1,4]diazepin-6(7H)-one (0.8 g, 2.53 mmol) and 4-amino-3-hydroxybenzoic acid (348.11 mg, 2.27 mmol) in EtOH (16 mL) and H₂O (48 mL) was added HCl (12 M, 463.06 μL) and the resulting mixture was stirred at 100° C. for 12 h. LCMS showed that the reaction was completed. The reaction mixture was concentrated, the residue was triturated with EtOAc (20 mL) and filtered, the filter cake was collected to afford 4-((9-cyclopentyl-7,7-difluoro-5-methyl-6-oxo-6,7,8,9-tetrahydro-5H-pyrimido[4,5-b][1,4]diazepin-2-yl)amino)-3-hydroxybenzoic acid (0.7 g, 1.50 mmol, 59.47% yield, 93% purity) as brown solid. MS(M+H)⁺=434.1

### Step 2. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-hydroxybenzamide (Compound 72)

To the solution of 4-((9-cyclopentyl-7,7-difluoro-5-methyl-6-oxo-6,7,8,9-tetrahydro-5H-pyrimido[4,5-b][1,4]diazepin-2-yl)amino)-3-hydroxybenzoic acid (150 mg, 346.09 μmol) and 4-(4-(3-(4-aminopiperidin-1-yl)-3-oxopropyl)piperazin-1-yl)-2-(2,6-dioxopiperidin-3-yl)isoindoline-1,3-dione (202.92 mg, 380.70 μmol, HCl) in DMF (2 mL) was added HOBt (70.15 mg, 519.14 μmol), EDCI (99.52 mg, 519.14 μmol) and TEA (105.06 mg, 1.04 mmol, 144.52 μL) and the resulting mixture was stirred at 20° C. for 12 h. LCMS showed that the reaction was completed, the mixture was poured into water (20 mL) and extracted with EtOAc (20 mL×3). The combined organic layer was washed with brine (50 mL), dried over Na₂SO₄, filtered and concentrated. The residue was purified by prep-HPLC (column: Phenomenex luna C₁₈ 150*25 mm*10 μm; mobile phase: [water(FA)-ACN]; B %: 13%-43%, 10 min) and the eluent lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-hydroxybenzamide (136 mg, 137.71 μmol, 39.79% yield, 97% purity, FA) as yellow solid. MS(M+H)⁺=912.1

¹H NMR (400 MHz, DMSO-d₆) δ=11.09 (s, 1H), 10.34 (br s, 1H), 8.26 (s, 1H), 8.18 (s, 1H), 8.15 (d, J=8.4 Hz, 1H), 8.10 (d, J=7.8 Hz, 1H), 7.98 (s, 1H), 7.71 (dd, J=7.3, 8.4 Hz, 1H), 7.40-7.32 (m, 4H), 5.10 (dd, J=5.3, 12.8 Hz, 1H), 4.79 (t, J=8.1 Hz, 1H), 4.38 (d, J=12.4 Hz, 1H), 4.12-3.90 (m, 5H), 3.35-3.32 (m, 4H), 3.15-3.11 (m, 1H), 2.93-2.85 (m, 1H), 2.66-2.53 (m, 12H), 2.07-1.78 (m, 6H), 1.74-1.71 (m, 2H), 1.67-1.58 (m, 4H), 1.49-1.33 (m, 2H).

### Example 73. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-(2-hydroxyethoxy)benzamide (Compound 73)

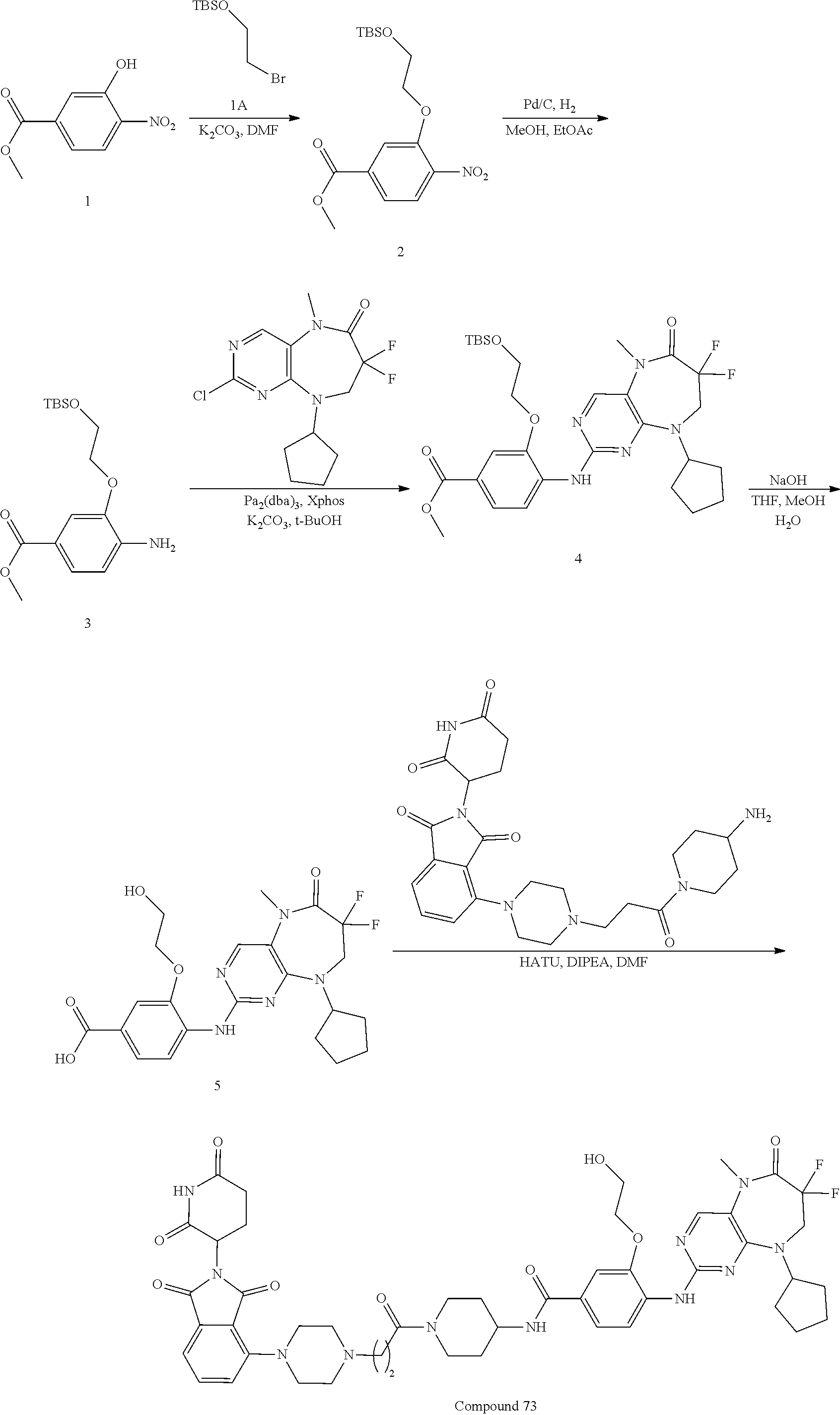

### Step 1. Synthesis of methyl 3-(2-((tert-butyldimethylsilyl)oxy)ethoxy)-4-nitrobenzoate (2)

To a solution of methyl 3-hydroxy-4-nitrobenzoate (1 g, 5.07 mmol) in DMF (10 mL) was added K₂CO₃ (1.40 g, 10.14 mmol), the mixture was stirred at 15° C. for 30 minutes, then to the mixture was added a solution of (2-bromoethoxy)(tert-butyl)dimethylsilane (1.82 g, 7.61 mmol) in DMF (5 mL) slowly at 15° C., the resulting mixture was heated to 100° C. for 4 hours under N₂ atmosphere. TLC (SiO₂, Petroleum ether:EtOAc=5:1) indicated the starting material was consumed completely and one major new spot with lower polarity was detected. The mixture was diluted with H₂O (200 mL) and extracted with EtOAc (100 mL×2). The combined organic layers were washed with brine (100 mL×5), dried over Na₂SO₄, filtered and concentrated in vacuo. The residue was purified by flash silica gel chromatography (20 g SepaFlash® Silica Flash Column, Eluent of 5˜80% EtOAc/Petroleum ether gradient @100 mL/min) to afford methyl 3-(2-((tert-butyldimethylsilyl)oxy)ethoxy)-4-nitrobenzoate (1.5 g, 4.22 mmol, 83.19% yield) as a off-white solid. MS(M+H)⁺=356.5

### Step 2. Synthesis of methyl 4-amino-3-(2-((tert-butyldimethylsilyl)oxy)ethoxy)benzoate (3)

To a solution of methyl 3-(2-((tert-butyldimethylsilyl)oxy)ethoxy)-4-nitrobenzoate (1.5 g, 4.22 mmol) in MeOH (50 mL) and EtOAc (20 mL) was added Pd/C (1 g, 4.22 mmol, 10% purity) under N₂ atmosphere. The suspension was stirred at 15° C. for 48 hours under H₂ (15 psi) atmosphere. LCMS showed the starting material was consumed completely and 89% of desired mass was detected. The mixture was degassed under vacuum and purged with N₂ several times, the mixture was filtered and the filtrate was concentrated in vacuo to afford methyl 4-amino-3-(2-((tert-butyldimethylsilyl)oxy)ethoxy)benzoate (1.5 g) as a brown solid, which was used for the next step directly. MS(M+H)⁺=326.1

### Step 3. Synthesis of methyl 3-(2-((tert-butyldimethylsilyl)oxy)ethoxy)-4-((9-cyclopentyl-7,7-difluoro-5-methyl-6-oxo-6,7,8,9-tetrahydro-5H-pyrimido[4,5-b][1,4]diazepin-2-yl)amino)benzoate (4)

To a solution of methyl 4-amino-3-(2-((tert-butyldimethylsilyl)oxy)ethoxy)benzoate (200 mg, 614.49 μmol), 2-chloro-9-cyclopentyl-7,7-difluoro-5-methyl-8,9-dihydro-5H-pyrimido[4,5-b][1,4]diazepin-6(7H)-one (175.17 mg, 553.04 μmol), Pd₂(dba)₃ (56.27 mg, 61.45 μmol) and XPhos (43.94 mg, 92.17 μmol) in t-BuOH (5 mL) was added K₂CO₃ (339.70 mg, 2.46 mmol), the suspension was stirred at 100° C. under N₂ atmosphere for 4 hours. LCMS showed 12% of methyl 4-amino-3-(2-((tert-butyldimethylsilyl)oxy)ethoxy)benzoate remained and 47% of desired mass was detected. The reaction mixture was concentrated in vacuo. The residue was purified by flash silica gel chromatography (12 g SepaFlash® Silica Flash Column, Eluent of 4˜15% EtOAc/Petroleum ether gradient @100 mL/min) to afford methyl 3-(2-((tert-butyldimethylsilyl)oxy)ethoxy)-4-((9-cyclopentyl-7,7-difluoro-5-methyl-6-oxo-6,7,8,9-tetrahydro-5H-pyrimido[4,5-b][1, 4]diazepin-2-yl)amino)benzoate (220 mg, 363.19 μmol, 59.10% yield) as a white solid. MS(M+H)⁺=606.2

### Step 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-3-(2-hydroxyethoxy)benzoic acid (5)

To a solution of methyl 3-(2-((tert-butyldimethylsilyl)oxy)ethoxy)-4-((9-cyclopentyl-7,7-difluoro-5-methyl-6-oxo-6,7,8,9-tetrahydro-5H-pyrimido[4,5-b][1,4]diazepin-2-yl)amino)benzoate (210 mg, 346.68 μmol) in MeOH (2 mL) and THF (2 mL) was added a solution of NaOH (277.32 mg, 6.93 mmol) in H₂O (2 mL), the mixture was stirred at 25° C. for 16 hours. LCMS showed the starting material was consumed completely and 72% of desired mass of 4-((9-cyclopentyl-7,7-difluoro-5-methyl-6-oxo-6,7,8,9-tetrahydro-5H-pyrimido[4,5-b][1,4]diazepin-2-yl)amino)-3-(2-hydroxyethoxy)benzoic acid was detected. The mixture was concentrated in vacuo. The residue was diluted with H₂O (8 mL) and extracted with EtOAc (10 mL×2). To the water phase was added HCl (12 M in water) to afford pH<3, the suspension was filtered, the filter cake was washed with H₂O (15 mL) and the filter cake was collected and dried to afford 4-[(9-cyclopentyl-7,7-difluoro-5-methyl-6-oxo-8H-pyrimido[4,5-b][1,4]diazepin-2-yl)amino]-3-(2-hydroxyethoxy)benzoic acid (130 mg) as a orange solid, which was used for the next step directly. MS(M+H)⁺=478.1.

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-(2-hydroxyethoxy)benzamide (Compound 73)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-(2-hydroxyethoxy)benzoic acid (65 mg, 136.14 μmol) in DMF (2 mL) were added HATU (77.65 mg, 204.21 μmol) and DIEA (105.57 mg, 816.82 μmol, 142.28 μL), the mixture was stirred at 15° C. for 15 minutes, to the mixture was added 4-(4-(3-(4-aminopiperidin-1-yl)-3-oxopropyl)piperazin-1-yl)-2-(2,6-dioxopiperidin-3-yl)isoindoline-1,3-dione (94.33 mg, 176.98 μmol, HCl), the resulting mixture was stirred at 15° C. for 1 hour. LCMS showed trace of 4-((9-cyclopentyl-7,7-difluoro-5-methyl-6-oxo-6,7,8,9-tetrahydro-5H-pyrimido[4,5-b][1,4]diazepin-2-yl)amino)-3-(2-hydroxyethoxy)benzoic acid remained and 88% of desired mass was detected. To the mixture was added CH₃COOH to adjust pH<7. The mixture was purified by prep-HPLC (column: Waters Xbridge 150*25 mm*5 μm; mobile phase: [water (10 mM NH₄HCO₃)-ACN]; B %: 31%-61%, min) followed by prep-HPLC (column: Phenomenex luna C₁₈ 150*25 mm*10 μm; mobile phase: [water (0.225% FA)-ACN]; B %: 15%-45%, 10 min), the eluent was freeze-dried to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-(2-hydroxyethoxy)benzamide (26.3 mg, 26.96 μmol, 19.80% yield, 98% purity) as a yellow solid. MS(M+H)⁺=956.3

¹H NMR (400 MHz, DMSO-d₆) δ=11.22-10.90 (m, 1H), 8.41 (s, 1H), 8.36-8.22 (m, 2H), 8.13 (d, J=7.8 Hz, 1H), 7.70 (dd, J=7.3, 8.3 Hz, 1H), 7.54-7.43 (m, 2H), 7.35 (t, J=7.2 Hz, 2H), 5.22 (s, 1H), 5.09 (dd, J=5.4, 12.8 Hz, 1H), 4.77 (q, J=7.8 Hz, 1H), 4.39 (d, J=13.2 Hz, 1H), 4.11 (t, J=4.6 Hz, 3H), 3.95 (d, J=12.9 Hz, 1H), 3.78 (d, J=3.4 Hz, 2H), 3.33 (bs, 3H), 3.30-3.29 (m, 4H), 3.13 (t, J=12.0 Hz, 1H), 2.94-2.81 (m, 1H), 2.70-2.52 (m, 11H), 2.08-1.99 (m, 1H), 1.99-1.90 (m, 2H), 1.90-1.77 (m, 2H), 1.77-1.67 (m, 2H), 1.66-1.55 (m, 4H), 1.53-1.34 (m, 2H).

### Example 74. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-(trifluoromethoxy)benzamide (Compound 74)

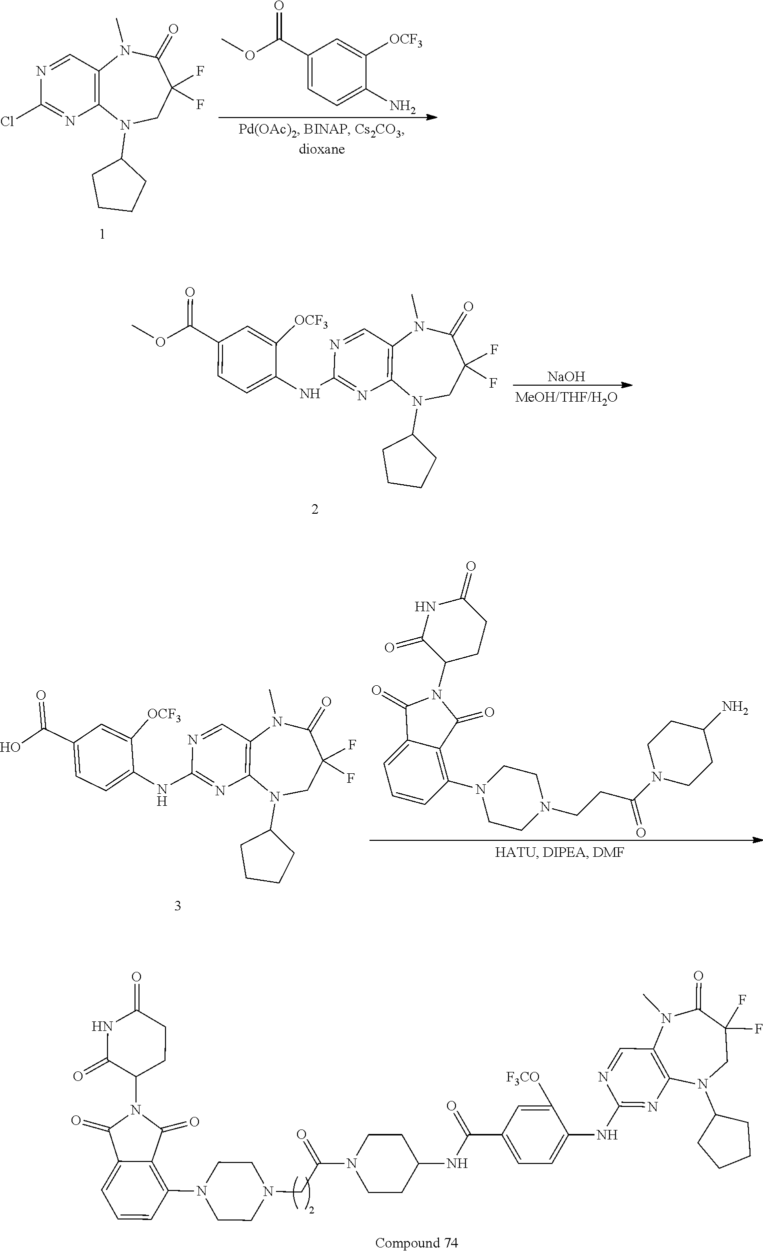

### Step 1. Synthesis of methyl 4-((9-cyclopentyl-7,7-difluoro-5-methyl-6-oxo-6,7,8,9-tetrahydro-5H-pyrimido[4,5-b][1,4]diazepin-2-yl)amino)-3-(trifluoromethoxy)benzoate (2)

A mixture of 2-chloro-9-cyclopentyl-7,7-difluoro-5-methyl-8,9-dihydro-5H-pyrimido[4,5-b][1,4]diazepin-6(7H)-one (200 mg, 631.45 μmol), methyl 4-amino-3-(trifluoromethoxy)benzoate (148.49 mg, 631.45 μmol), BINAP (78.64 mg, 126.29 μmol), Cs₂CO₃ (617.21 mg, 1.89 mmol) and Pd(OAc)₂ (14.18 mg, 63.14 μmol) in dioxane (2 mL) was de-gassed N₂ and then heated to 100° C. for 12 h under N₂. LCMS showed a peak (58%) with desired mass. The reaction mixture was diluted with H₂O (10 mL) and extracted with EtOAc (15 mL×3). The combined organic layers were washed with brine (15 mL), dried over Na₂SO₄, filtered and concentrated under reduced pressure to give a residue. The residue was purified by flash silica gel chromatography (Biotage, 12 g SepaFlash® Silica Flash Column, Eluent of 20˜40% Petroleum ether/EtOAc gradient @60 mL/min) to afford methyl 4-((9-cyclopentyl-7,7-difluoro-5-methyl-6-oxo-6,7,8,9-tetrahydro-5H-pyrimido[4,5-b][1,4]diazepin-2-yl)amino)-3-(trifluoromethoxy)benzoate (210 mg, 407.42 μmol, 64.52% yield) as white solid. MS(M+H)⁺=516.2

### Step 2. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-3-(trifluoromethoxy)benzoic acid (3)

To a solution of methyl 4-((9-cyclopentyl-7,7-difluoro-5-methyl-6-oxo-6,7,8,9-tetrahydro-5H-pyrimido[4,5-b][1,4]diazepin-2-yl)amino)-3-(trifluoromethoxy)benzoate (210 mg, 407.42 μmol) in THF (1 mL) and MeOH (1 mL) was added a solution of NaOH (81.48 mg, 2.04 mmol) in H₂O (1 mL) dropwise, the mixture was stirred at 25° C. for 2 h. LCMS showed main peak with the mass [501+H₂O+H]⁺ was detected. The mixture was concentrated in vacuo and added 1N HCl to adjusted pH=3, the mixture was concentrated in vacuo and to afford 4-((9-cyclopentyl-7,7-difluoro-5-methyl-6-oxo-6,7,8,9-tetrahydro-5H-pyrimido[4,5-b][1,4]diazepin-2-yl)amino)-3-(trifluoromethoxy)benzoic acid (290 mg, crude) as white solid. MS(M+H)⁺=502.2

### Step 3. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-(trifluoromethoxy)benzamide (Compound 74)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-(trifluoromethoxy)benzoic acid (50 mg, 99.72 μmol) in DMF (1 mL) were added HATU (75.83 mg, 199.44 μmol) and DIPEA (38.66 mg, 299.16 μmol, 52.11 μL), the mixture was stirred at 25° C. for 10 min, then 4-(4-(3-(4-aminopiperidin-1-yl)-3-oxopropyl)piperazin-1-yl)-2-(2,6-dioxopiperidin-3-yl)isoindoline-1,3-dione (49.52 mg, 92.90 μmol, HCl) was added, the mixture was stirred at 25° C. for another 1 h. LCMS showed a peak (77%) with desired mass. The mixture was purified by prep-HPLC (column: Waters Xbridge 150*25 mm*5 μm; mobile phase: [water (10 mM NH₄HCO₃)-ACN]; B %: 41%-71%, min) followed by lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-(trifluoromethoxy)benzamide (26.9 mg, 26.08 μmol, 26.15% yield, 95.0% purity) as yellow solid. MS(M+H)⁺=980.3

¹H NMR (400 MHz, DMSO-d₆) δ=11.10 (s, 1H), 9.01 (s, 1H), 8.38-8.32 (m, 1H), 8.25 (s, 1H), 8.15 (d, J=9.0 Hz, 1H), 7.89-7.83 (m, 2H), 7.73-7.67 (m, 1H), 7.36 (t, J=7.2 Hz, 2H), 5.13-5.06 (m, 1H), 4.73-4.62 (m, 1H), 4.43-4.34 (m, 1H), 4.10-3.91 (m, 4H), 3.33-3.32 (m, 6H), 3.17-3.10 (m, 1H), 2.92-2.83 (m, 1H), 2.67-2.53 (m, 12H), 2.07-1.98 (m, 1H), 1.91-1.80 (m, 4H), 1.71-1.62 (m, 2H), 1.60-1.46 (m, 5H), 1.43-1.32 (m, 1H)

### Example 75. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-(trifluoromethyl)benzamide (Compound 75)

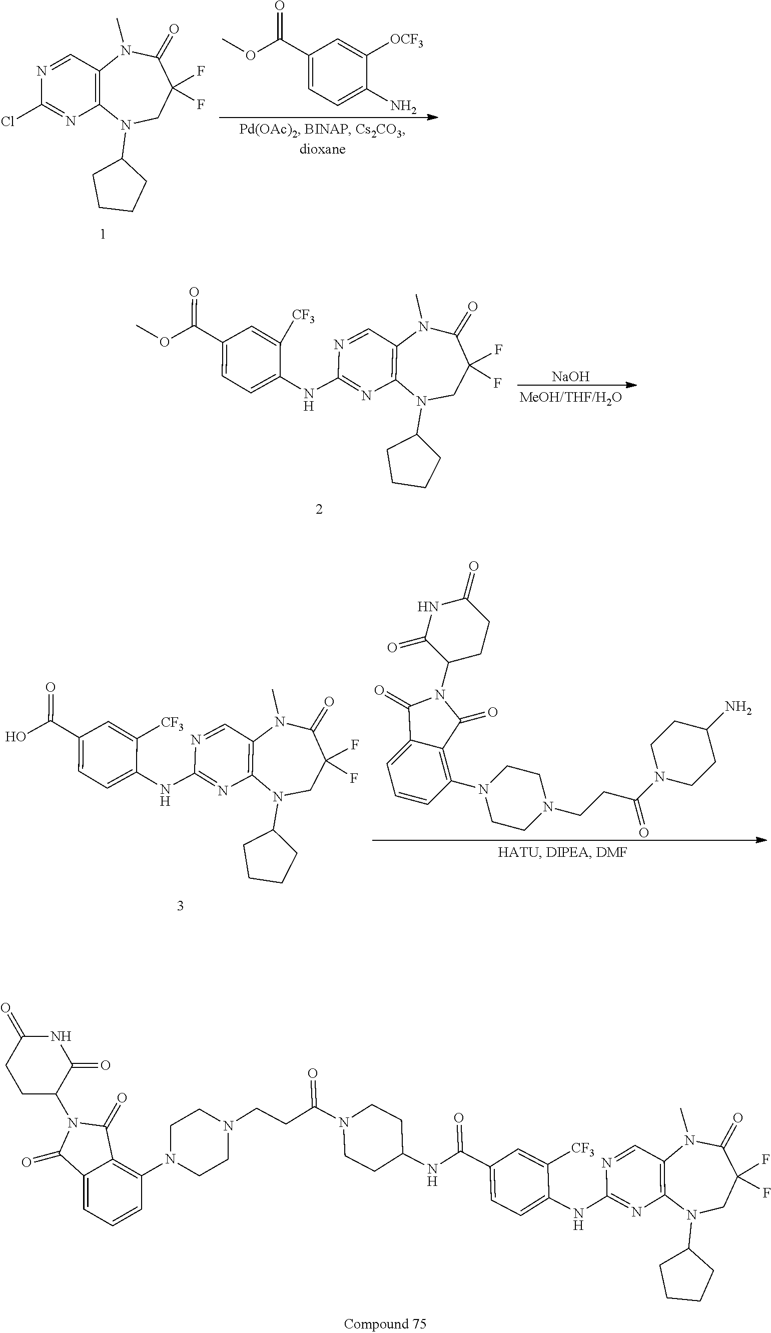

### Step 1. Synthesis of methyl 4-((9-cyclopentyl-7,7-difluoro-5-methyl-6-oxo-6,7,8,9-tetrahydro-5H-pyrimido[4,5-b][1,4]diazepin-2-yl)amino)-3-(trifluoromethyl)benzoate (2)

A mixture of 2-chloro-9-cyclopentyl-7,7-difluoro-5-methyl-8,9-dihydro-5H-pyrimido[4,5-b][1,4]diazepin-6(7H)-one (150 mg, 473.58 μmol), methyl 4-amino-3-(trifluoromethyl)benzoate (114.17 mg, 520.94 μmol), Cs₂CO₃ (462.91 mg, 1.42 mmol), BINAP (58.98 mg, 94.72 μmol) and Pd(OAc)₂ (10.63 mg, 47.36 μmol) in dioxane (5 mL) was de-gassed N₂ and then heated at 100° C. for 12 h under N₂. LCMS showed a peak (41%) with desired mass. The reaction mixture was diluted with H₂O (10 mL) and extracted with EtOAc (20 mL×2). The combined organic layers were washed with brine (20 mL), dried over Na₂SO₄, filtered and concentrated under reduced pressure to give a residue. The residue was purified by flash silica gel chromatography (Biotage, 20 g SepaFlash® Silica Flash Column, Eluent of 10˜30% Petroleum ether/EtOAc gradient @60 mL/min) to afford methyl 4-((9-cyclopentyl-7,7-difluoro-5-methyl-6-oxo-6,7,8,9-tetrahydro-5H-pyrimido[4,5-b][1,4]diazepin-2-yl)amino)-3-(trifluoromethyl)benzoate (118 mg, 236.27 μmol, 49.89% yield) as white solid. MS(M+H)⁺=500.1

### Step 2. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-3-(trifluoromethyl)benzoic acid (3)

To a solution of methyl 4-((9-cyclopentyl-7,7-difluoro-5-methyl-6-oxo-6,7,8,9-tetrahydro-5H-pyrimido[4,5-b][1,4]diazepin-2-yl)amino)-3-(trifluoromethyl)benzoate (118 mg, 236.27 μmol) in THF (1 mL) and MeOH (1 mL) was added a solution of NaOH (47.25 mg, 1.18 mmol) in H₂O (1 mL) dropwise, the mixture was stirred at 25° C. for 2 h. LCMS showed main peak with the mass [485+H₂O+H]⁺ was detected. The mixture was concentrated in vacuo and added 1N HCl to adjusted pH=3, the mixture was concentrated in vacuo and to afford 4-((9-cyclopentyl-7,7-difluoro-5-methyl-6-oxo-6,7,8,9-tetrahydro-5H-pyrimido[4,5-b][1,4]diazepin-2-yl)amino)-3-(trifluoromethyl)benzoic acid (110 mg, crude) as white solid. MS(M+H)⁺=486.1

### Step 3. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-(trifluoromethyl)benzamide (Compound 75)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-(trifluoromethyl)benzoic acid (50 mg, 103.01 μmol) and 4-(4-(3-(4-aminopiperidin-1-yl)-3-oxopropyl)piperazin-1-yl)-2-(2,6-dioxopiperidin-3-yl)isoindoline-1,3-dione (54.90 mg, 103.01 μmol, HCl) in DMF (1 mL) were added HATU (78.33 mg, 206.01 μmol) and DIPEA (39.94 mg, 309.02 μmol, 53.83 μL), the mixture was stirred at 25° C. for 1 h. LCMS showed a peak (71%) with desired mass. The mixture was purified by prep-HPLC (column: Phenomenex Synergi C₁₈ 150*25 mm*10 μm; mobile phase: [water (0.225% FA)-ACN]; B %: 19%-55%, 12 min) and prep-HPLC (column: Waters Xbridge 150*25 mm*5 μm; mobile phase: [water (10 mM NH₄HCO₃)-ACN]; B %: 40%-70%, min) followed by lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-(trifluoromethyl)benzamide (10.6 mg, 10.85 μmol, 10.53% yield, 98.65% purity) as yellow solid. MS(M+H)⁺=964.4

¹H NMR (400 MHz, DMSO-d₆) δ=11.15-11.02 (m, 1H), 8.58-8.44 (m, 2H), 8.19 (s, 2H), 8.12 (d, J=9.1 Hz, 1H), 7.99-7.93 (m, 1H), 7.70 (t, J=7.8 Hz, 1H), 7.35 (t, J=7.3 Hz, 2H), 5.13-5.05 (m, 1H), 4.54-4.44 (m, 1H), 4.42-4.32 (m, 1H), 4.11-3.91 (m, 4H), 3.32-3.31 (m, 6H), 3.19-3.09 (m, 1H), 2.93-2.81 (m, 1H), 2.63-2.54 (m, 12H), 2.06-1.97 (m, 1H), 1.92-1.79 (m, 2H), 1.79-1.70 (m, 2H), 1.63-1.56 (m, 2H), 1.55-1.46 (m, 3H), 1.43-1.35 (m, 3H).

### Example 76. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(3-(1-(2-(2,6-dioxopiperidin-3-yl)-1,3-dioxoisoindolin-4-yl)piperidin-4-yl)propanoyl)piperazin-1-yl)-3-methoxybenzamide (Compound 76)

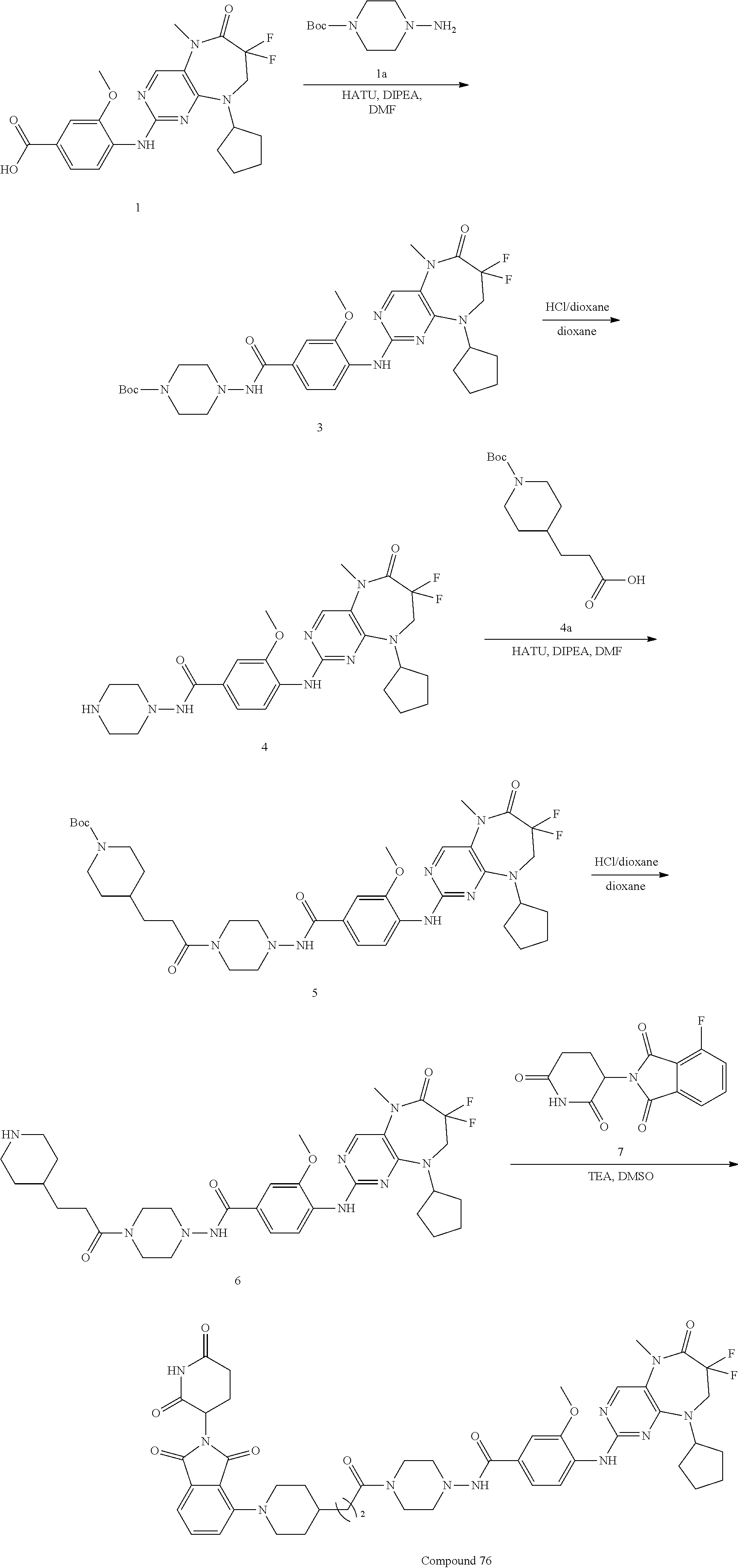

### Step 1. Synthesis of tert-butyl 4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperazine-1-carboxylate (3)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (0.5 g, 1.12 mmol) in DMF (2 mL) was added HATU (637.35 mg, 1.68 mmol) and DIPEA (433.27 mg, 3.35 mmol, 583.92 μL). The mixture was stirred at 30° C. for 10 min. To mixture was added tert-butyl 4-aminopiperazine-1-carboxylate (247.40 mg, 1.23 mmol). The mixture was stirred at 30° C. for 12 h. LCMS showed the 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid was consumed completely and a peak (42%) with desired mass. The mixture was diluted with EtOAc (20 mL) and concentrated under reduced pressure. The residue was purified by flash silica gel chromatography (5 g SepaFlash® Silica Flash Column, Eluent of 0˜100% EtOAc/Petroleum ether gradient @50 mL/min) to afford tert-butyl 4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperazine-1-carboxylate (0.8 g, crude) as brown oil, which was used for the next step directly. MS(M+H)⁺=631.2

### Step 2. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(piperazin-1-yl)benzamide (4)

To a solution of tert-butyl 4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperazine-1-carboxylate (0.8 g, 1.27 mmol) in dioxane (1 mL) was added HCl/dioxane (4 M, 10 mL) at 25° C. The resulting mixture was stirred at 25° C. for 0.5 hr. LCMS showed the starting material was consumed completely, and a main peak with desired mass. The mixture solution was concentrated under reduced pressure to afford the 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(piperazin-1-yl)benzamide (0.9 g, crude, HCl) as brown solid, which was used for the next step directly. MS(M+H)⁺=531.1

### Step 3. Synthesis of tert-butyl 4-(3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperazin-1-yl)-3-oxo propyl)piperidine-1-carboxylate (5)

To a solution of 3-(1-(tert-butoxycarbonyl)piperidin-4-yl)propanoic acid (300 mg, 1.17 mmol) in DMF (4 mL) was added HATU (664.93 mg, 1.75 mmol) and DIPEA (452.02 mg, 3.50 mmol, 609.19 μL). The mixture was stirred at 25° C. for 10 min. To mixture was added 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(piperazin-1-yl)benzamide (661.07 mg, 1.17 mmol, HCl). The mixture was stirred at 25° C. for 12 h. LCMS showed the 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(piperazin-1-yl)benzamide was consumed completely and a peak (68%) with desired mass. The mixture was diluted with EtOAc (30 mL) and concentrated. The residue was purified by flash silica gel chromatography (12 g SepaFlash® Silica Flash Column, Eluent of 0˜100% EtOAc/Petroleum ether gradient @50 mL/min; Eluent of 0˜50% Methanol/EtOAc @50 mL/min) to afford tert-butyl 4-(3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperazin-1-yl)-3-oxopropyl) piperidine-1-carboxylate as brown solid, which was used for the next step directly. MS(M+H)⁺=770.1

### Step 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(4-(3-(piperidin-4-yl)propanoyl)piperazin-1-yl)benzamide (6)

To a solution of tert-butyl 4-(3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperazin-1-yl)-3-oxopropyl) piperidine-1-carboxylate (0.5 g, 649.45 μmol) in dioxane (2 mL) was added HCl/dioxane (4 M, 20 mL) at 25° C. The resulting mixture was stirred at 25° C. for 0.5 hr. LCMS showed the starting material was consumed completely, and a main peak with desired mass. The mixture solution was concentrated under reduced pressure to give 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(4-(3-(piperidin-4-yl)propanoyl)piperazin-1-yl)benzamide (0.5 g, crude, HCl) as brown solid, which was used for the next step directly. MS(M+H)⁺=670.4

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(3-(1-(2-(2,6-dioxopiperidin-3-yl)-1,3-dioxoisoindolin-4-yl)piperidin-4-yl)propanoyl)piperazin-1-yl)-3-methoxybenzamide (Compound 76)

A solution of 2-(2,6-dioxopiperidin-3-yl)-4-fluoroisoindoline-1,3-dione (234.67 mg, 849.59 μmol), 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(4-(3-(piperidin-4-yl)propanoyl)piperazin-1-yl)benzamide (500 mg, 707.99 μmol, HCl) and TEA (214.92 mg, 2.12 mmol, 295.63 μL) in DMSO (5 mL) was stirred at 90° C. for 12 hr. LCMS showed the 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(4-(3-(piperidin-4-yl)propanoyl)piperazin-1-yl)benzamide was consumed completely and a peak (33%) with desired mass. The crude product was purified by prep-HPLC (column: Phenomenex luna C18 150*40 mm*15 μm; mobile phase: [water(TFA)-ACN]; B %: 32%-62%, 11 min) and re-purified by prep-HPLC (column: Waters Xbridge C18 150*50 mm*10 μm; mobile phase: [water(NH₄HCO₃)-ACN]; B %: 34%-64%, 11 min) to give the product. The product was combined with other batch and dissolved with cold deionized water (10 mL), ACN (30 mL) and lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(3-(1-(2-(2,6-dioxopiperidin-3-yl)-1,3-dioxoisoindolin-4-yl)piperidin-4-yl)propanoyl)piperazin-1-yl)-3-methoxybenzamide (227.4 mg, 0.241 mmol, 98% purity) as yellow solid. MS(M+H)⁺=926.5

¹H NMR (400 MHz, DMSO-d₆) δ=11.08 (s, 1H), 9.46 (s, 1H), 8.32-8.23 (m, 2H), 7.97 (s, 1H), 7.68 (dd, J=7.1, 8.4 Hz, 1H), 7.46-7.39 (m, 2H), 7.33 (t, J=7.4 Hz, 2H), 5.09 (dd, J=5.4, 12.8 Hz, 1H), 4.84-4.69 (m, 1H), 4.04 (t, J=14.0 Hz, 2H), 3.93 (s, 3H), 3.70 (d, J=11.1 Hz, 2H), 3.57 (d, J=1.0 Hz, 3H), 3.30 (s, 3H), 3.02-2.78 (m, 7H), 2.64-2.53 (m, 2H), 2.41 (t, J=7.3 Hz, 2H), 2.12-1.26 (m, 17H)

### Example 77. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxy-N-methylbenzamide (Compound 77)

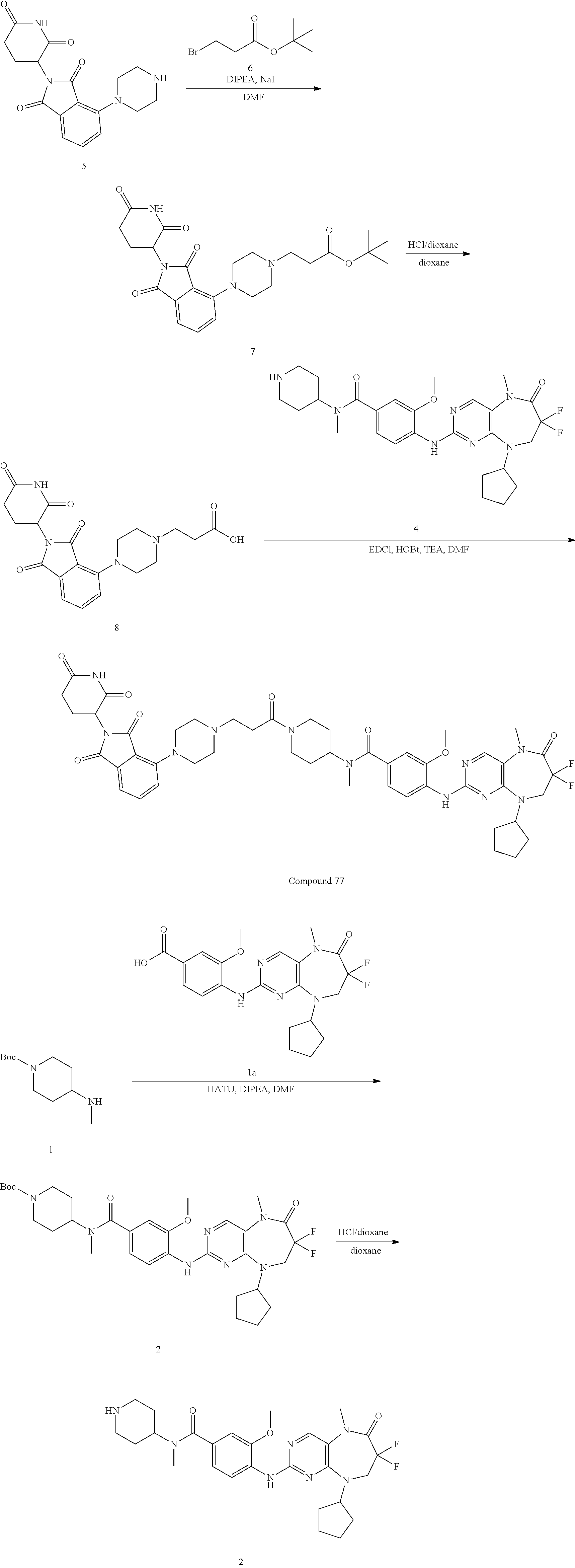

### Step 1. Synthesis of tert-butyl 4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-methylbenzamido)piperidine-1-carboxylate (2)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (100 mg, 223.50 μmol) in DMF (2 mL) was added HATU (127.47 mg, 335.24 μmol) and DIPEA (86.65 mg, 670.49 μmol, 116.78 μL). The mixture was stirred at 25° C. for 10 min. To mixture was added tert-butyl 4-(methylamino)piperidine-1-carboxylate (47.90 mg, 223.50 μmol). The mixture was stirred at 25° C. for 12 h. LCMS showed the 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid was consumed completely and a main peak with desired mass. The reaction mixture was purified by prep-HPLC (column: Phenomenex Synergi Polar-RP 100×25 mm×4 um; mobile phase: [water (TFA)-ACN]; B %: 50%-70%, 7 min) and lyophilized to afford tert-butyl 4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-methylbenzamido)piperidine-1-carboxylate (120 mg, 178.96 μmol, 80.07% yield, 96% purity) as brown solid, which was used for the next step directly. MS(M+H)⁺=644.4

### Step 2. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-methyl-N-(piperidin-4-yl)benzamide (3)

To a solution of tert-butyl 4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-methylbenzamido)piperidine-1-carboxylate (120 mg, 186.42 μmol) in dioxane (1 mL) was added HCl/dioxane (4 M, 10 mL) at 25° C. The resulting mixture was stirred at 25° C. for 0.5 hr. LCMS showed the starting material was consumed completely and a main peak with desired mass. The mixture was concentrated under reduced pressure to afford 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-methyl-N-(piperidin-4-yl)benzamide (100 mg, crude) as brown solid, which was used for the next step directly. MS(M+H)⁺=544.2

### Step 3. Synthesis of tert-butyl 3-(4-(2-(2,6-dioxopiperidin-3-yl)-1,3-dioxoisoindolin-4-yl)piperazin-1-yl)propanoate (7)

A mixture 2-(2,6-dioxopiperidin-3-yl)-4-(piperazin-1-yl)isoindoline-1,3-dione (0.2 g, 584.20 μmol), tert-butyl 3-bromopropanoate (122.14 mg, 584.20 μmol, 97.72 μL), DIEA (226.51 mg, 1.75 mmol, 305.26 μL) and NaI (8.76 mg, 58.42 μmol) in DMF (4 mL) was stirred at 80° C. for 2 h. LCMS showed the starting material was consumed completely and a main peak with desired mass. The mixture solution was concentrated under reduced pressure. The residue was purified by flash silica gel chromatography (25 g SepaFlash® Silica Flash Column, Eluent of 0˜100% EtOAc/Petroleum ether gradient @70 mL/min; Eluent of 0˜50% Methanol/EtOAc @70 mL/min) to afford tert-butyl 3-(4-(2-(2,6-dioxopiperidin-3-yl)-1,3-dioxoisoindolin-4-yl)piperazin-1-yl)propanoate (0.3 g, crude) as yellow solid, which was used for the next step directly. MS (M+H)⁺=471.2

### Step 4. Synthesis of 3-(4-(2-(2,6-dioxopiperidin-3-yl)-1,3-dioxoisoindolin-4-yl)piperazin-1-yl)propanoic acid (8)

To a solution of tert-butyl 3-(4-(2-(2,6-dioxopiperidin-3-yl)-1,3-dioxoisoindolin-4-yl)piperazin-1-yl)propanoate (0.3 g, 637.60 μmol) in dioxane (1 mL) was added HCl/dioxane (4 M, 10 mL) at 25° C. The resulting mixture was stirred at 25° C. for 0.5 hr. TLC (Dichloromethane:Methanol=10:1; Rf=0.5) showed the starting material was consumed completely and found new spot. The mixture solution was concentrated under reduced pressure to give 3-(4-(2-(2,6-dioxopiperidin-3-yl)-1,3-dioxoisoindolin-4-yl)piperazin-1-yl)propanoic acid (260 mg, crude) as yellow solid, which was used for the next step directly. MS(M+H)⁺=415.0

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxy-N-methylbenzamide (Compound 77)

To a mixture of 3-(4-(2-(2,6-dioxopiperidin-3-yl)-1,3-dioxoisoindolin-4-yl)piperazin-1-yl)propanoic acid (50 mg, 120.65 μmol) and 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-methyl-N-(piperidin-4-yl)benzamide (69.99 mg, 120.65 μmol, HCl) in DMF (1 mL) was added HOBt (17.93 mg, 132.72 μmol), EDCI (25.44 mg, 132.72 μmol) and TEA (36.63 mg, 361.96 μmol, 50.38 μL, 3 eq) at 25° C. The mixture was stirred at 25° C. for 2 h. LCMS showed the 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-methyl-N-(piperidin-4-yl)benzamide was consumed completely and a main peak with desired mass. The mixture was poured into water (10 mL) and extracted with EtOAc (20 mL×3). The combined organic phase was washed with brine (10 mL×3), dried over Na₂SO₄, filtered and concentrated under reduced pressure to give a residue. The residue was purified by prep-HPLC (column: Phenomenex Synergi Polar-RP 100×25 mm×4 μm; mobile phase: [water (TFA)-ACN]; B %: 31%-51%, 7 min) and re-purified by prep-HPLC (column: Waters Xbridge 150×25 mm×5 μm; mobile phase: [water(NH₄HCO₃)-ACN]; B %: 36%-66%, 9 min) and lyophilized to give 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxy-N-methylbenzamide (26 mg, 26.00 μmol, 21.55% yield, 94% purity) as yellow solid. MS(M+H)⁺=940.5

¹H NMR (400 MHz, CDCl₃) δ=8.45 (d, J=8.1 Hz, 1H), 8.23 (br s, 1H), 8.06 (s, 1H), 7.69 (s, 1H), 7.62 (t, J=7.8 Hz, 1H), 7.43 (d, J=7.1 Hz, 1H), 7.18 (d, J=8.4 Hz, 1H), 7.04-6.95 (m, 2H), 4.96 (dd, J=5.3, 12.1 Hz, 1H), 4.88-4.71 (m, 2H), 4.12-3.74 (m, 7H), 3.49-3.35 (m, 7H), 3.01-2.57 (m, 16H), 2.19-2.02 (m, 3H), 1.93-1.69 (m, 11H).

### Example 78. Synthesis of 2-chloro-4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-5-methoxybenzamide (Compound 78)

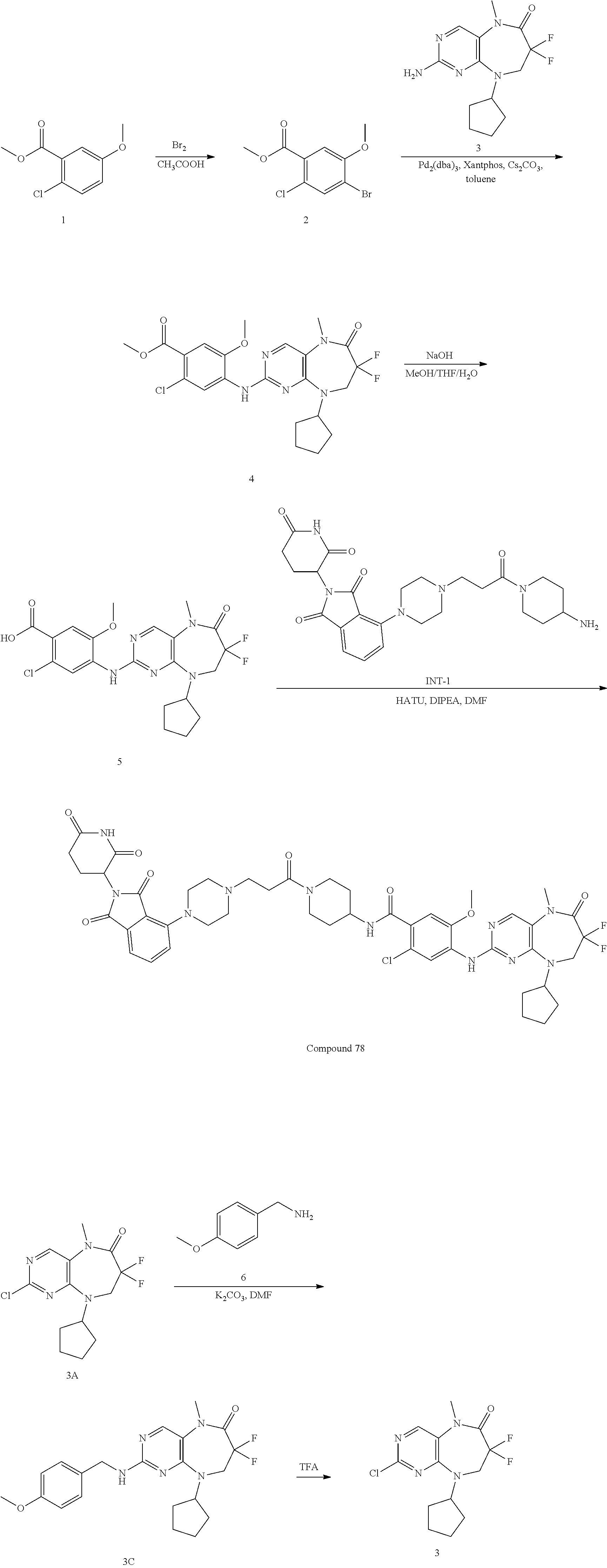

### Step 1. Synthesis of 9-cyclopentyl-7,7-difluoro-2-((4-methoxybenzyl)amino)-5-methyl-5,7,8,9-tetrahydro-6H-pyrimido[4,5-b][1,4]diazepin-6-one (3C)

To a solution of 2-chloro-9-cyclopentyl-7,7-difluoro-5-methyl-5,7,8,9-tetrahydro-6H-pyrimido[4,5-b][1,4]diazepin-6-one (2 g, 6.31 mmol) and (4-methoxyphenyl)methanamine (1.30 g, 9.47 mmol, 1.23 mL) in DMF (20 mL) was added K₂CO₃ (2.18 g, 15.79 mmol), the mixture was stirred at 100° C. for 16 hours under N₂ atmosphere. LCMS showed the 2-chloro-9-cyclopentyl-7,7-difluoro-5-methyl-5,7,8,9-tetrahydro-6H-pyrimido[4,5-b][1,4]diazepin-6-one was consumed completely and the desired mass. To the mixture was added H₂O (100 mL) and extracted with EtOAc (50 mL×3). The combined organic layers were washed with brine (20 mL×5), dried over Na₂SO₄, filtered and concentrated in vacuum. The residue was triturated with MTBE (5 mL) and EtOAc (5 mL) for 5 minutes, the suspension was filtered and the filter cake was washed with EtOAc (5 mL) and MTBE (10 mL) in turn, the filtrate was concentrated in vacuum to afford 9-cyclopentyl-7,7-difluoro-2-((4-methoxybenzyl)amino)-5-methyl-5,7,8,9-tetrahydro-6H-pyrimido[4,5-b][1,4]diazepin-6-one (1.5 g) as a yellow solid. MS(M+H)⁺=418.0

### Step 2. Synthesis of 2-amino-9-cyclopentyl-7,7-difluoro-5-methyl-5,7,8,9-tetrahydro-6H-pyrimido[4,5-b][1,4]diazepin-6-one (3)

A mixture of 9-cyclopentyl-7,7-difluoro-2-((4-methoxybenzyl)amino)-5-methyl-5,7,8,9-tetrahydro-6H-pyrimido[4,5-b][1,4]diazepin-6-one (1.5 g) in TFA (15 mL) was stirred at 25° C. for 16 hours. LCMS showed the starting material was consumed completely and desired mass. The reaction mixture was concentrated in vacuum. The residue was diluted with EtOAc (50 mL), the mixture was washed with Na₂CO₃ solution (sat, 30 mL), the organic phase was dried over Na₂SO₄, filtered and concentrated in vacuum. The residue was purified by prep-HPLC (column: Waters Xbridge C18 150×50 mm×10 μm; mobile phase: [water(NH₄HCO₃)-ACN]; B %: 24%-54%, 11 min) and freeze-dried to afford 2-amino-9-cyclopentyl-7,7-difluoro-5-methyl-5,7,8,9-tetrahydro-6H-pyrimido[4,5-b][1,4]diazepin-6-one (200 mg, 672.71 μmol, 28.08% yield) as a brown gum. MS(M+H)⁺=298.2

### Step 3. Synthesis of methyl 4-bromo-2-chloro-5-methoxybenzoate (2)

To a solution of methyl methyl 2-chloro-5-methoxybenzoate (2.6 g, 12.96 mmol) in CH₃COOH (14 mL) was added a solution of Br₂ (2.28 g, 14.26 mmol, 734.91 μL) in CH₃COOH (6 mL) at 0° C. The mixture was stirred at 25° C. for 12 hours. Then to the mixture was added a solution of Br₂ (2.07 g, 12.96 mmol, 668.10 μL) in CH₃COOH (4 mL) at 0° C. The resulting material was stirred at 25° C. for 16 hours. LCMS showed 25% of the starting material remained and 24% of desired mass was detected. To the mixture was added a solution of Br₂ (2.07 g, 12.96 mmol, 668.10 μL) in CH₃COOH (4 mL), the mixture was stirred at 25° C. for 12 hours, LCMS showed 8% of the starting material remained and 31% of desired mass. The mixture was poured into NaHCO₃ (300 mL, sat). The resulting mixture was extracted with EtOAc (100 mL×3). The combined organic layers were washed with NaHCO₃ (100 mL, sat), dried over Na₂SO₄, filtered and concentrated in vacuum. The residue was purified by pre-HPLC (column: Phenomenex luna C18 150×40 mm×15 μm; mobile phase: [water(FA)-ACN]; B %: 55%-65%, 10 min) and freeze-dried to afford methyl 4-bromo-2-chloro-5-methoxybenzoate (270 mg, 965.96 μmol, 7.45% yield) as a gray solid. MS(M+H)⁺=280.8

### Step 4. Synthesis of methyl 2-chloro-4-((9-cyclopentyl-7,7-difluoro-5-methyl-6-oxo-6,7,8,9-tetrahydro-5H-pyrimido[4,5-b][1,4]diazepin-2-yl)amino)-5-methoxybenzoate (4)

Under N₂ atmosphere, to a mixture of methyl 4-bromo-2-chloro-5-methoxybenzoate (130 mg, 465.09 μmol) and 2-amino-9-cyclopentyl-7,7-difluoro-5-methyl-5,7,8,9-tetrahydro-6H-pyrimido[4,5-b][1,4]diazepin-6-one (207.41 mg, 697.64 μmol) in toluene (20 mL) were added Pd₂(dba)₃ (42.59 mg, 46.51 μmol), Xantphos (40.37 mg, 69.76 μmol) and Cs₂CO₃ (454.61 mg, 1.40 mmol) under nitrogen atmosphere. The resulting mixture was stirred at 100° C. for 16 hours under N₂ atmosphere. LCMS showed methyl 4-bromo-2-chloro-5-methoxybenzoate was consumed completely and the desired mass. The reaction mixture was filtered and the filtrate was concentrated in vacuum. The residue was purified by prep-TLC (SiO₂, Petroleum ether:EtOAc=1:1) to afford methyl 2-chloro-4-((9-cyclopentyl-7,7-difluoro-5-methyl-6-oxo-6,7,8,9-tetrahydro-5H-pyrimido[4,5-b][1,4]diazepin-2-yl)amino)-5-methoxybenzoate (218 mg, 439.60 μmol, 94.52% yield) as a brown solid. MS(M+H)⁺=496.0

### Step 5. Synthesis of 2-chloro-4-((9-cyclopentyl-7,7-difluoro-5-methyl-6-oxo-6,7,8,9-tetrahydro-5H-pyrimido[4,5-b][1,4]diazepin-2-yl)amino)-5-methoxybenzoic acid (5)

To a solution of methyl 2-chloro-4-((9-cyclopentyl-7,7-difluoro-5-methyl-6-oxo-6,7,8,9-tetrahydro-5H-pyrimido[4,5-b][1,4]diazepin-2-yl)amino)-5-methoxybenzoate (215 mg, 433.55 μmol) in MeOH (3 mL), H2O (3 mL) and THF (3 mL) was added NaOH (2 M in H₂O, 0.7 mL), the resulting mixture was stirred at 25° C. for 16 hours. LCMS showed trace of the starting material remained. To the mixture was added NaOH (2 M in H₂O, 0.5 mL) and the resulting mixture was stirred at 25° C. for another 4 hours. LCMS showed trace of the starting material remained. The mixture was concentrated in vacuum to remove most of the solvent, the residue was diluted with H₂O (10 mL). To the suspension was added HCl solution (12 M) to adjust pH<3. To the mixture was added EtOAc (10 mL) and the suspension was stirred for 10 minutes. The suspension was filtered and the filter cake was washed with MTBE (20 mL), collected and dried. To the residue was added HCl solution (15 mL, 4 M) and the suspension was stirred for 5 minutes. The suspension was concentrated in vacuum to afford 2-chloro-4-((9-cyclopentyl-7,7-difluoro-5-methyl-6-oxo-6,7,8,9-tetrahydro-5H-pyrimido[4,5-b][1,4]diazepin-2-yl)amino)-5-methoxybenzoic acid (200 mg, 415.04 μmol, 95.73% yield) as a brown solid. MS(M+H)⁺=482.0

¹H NMR (400 MHz, DMSO-d6) δ=8.81 (br s, 1H), 8.36 (d, J=10.1 Hz, 2H), 7.47 (s, 1H), 5.00-4.85 (m, 1H), 4.18 (br t, J=13.2 Hz, 2H), 3.93 (s, 3H), 3.33 (s, 3H), 2.02-1.91 (m, 2H), 1.85-1.70 (m, 2H), 1.7-1.50 (m, 5H).

### Step 6. Synthesis of 2-chloro-4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-5-methoxybenzamide (Compound 78)

To a solution of 2-chloro-4-((9-cyclopentyl-7,7-difluoro-5-methyl-6-oxo-6,7,8,9-tetrahydro-5H-pyrimido[4,5-b][1,4]diazepin-2-yl)amino)-5-methoxybenzoic acid (70 mg, 145.26 μmol) in DMF (2 mL) were added HATU (82.85 mg, 217.90 μmol) and DIPEA (93.87 mg, 726.32 μmol, 126.51 μL), the mixture was stirred at 25° C. for 15 minutes. To the mixture was added 4-(4-(3-(4-aminopiperidin-1-yl)-3-oxopropyl)piperazin-1-yl)-2-(2,6-dioxopiperidin-3-yl)isoindoline-1,3-dione (100.66 mg, 188.84 μmol, HCl salt). The resulting mixture was stirred at 25° C. for 1 hour. LCMS showed 2-chloro-4-((9-cyclopentyl-7,7-difluoro-5-methyl-6-oxo-6,7,8,9-tetrahydro-5H-pyrimido[4,5-b][1,4]diazepin-2-yl)amino)-5-methoxybenzoic acid was consumed completely and the desired mass. The mixture was combined with another batch (20 mg). To the combined reaction mixture was added CH₃COOH to adjust pH<7. The mixture was purified by prep-HPLC (column: Phenomenex Synergi C18 150×25 mm×10 μm; mobile phase: [water(FA)-ACN]; B %: 15%-51%, 11 min) followed by prep-HPLC (column: Waters Xbridge 150×25 mm×5 μm; mobile phase: [water(NH₄HCO₃)-ACN]; B %: 45%-75%, 7 min) and freeze-dried to afford 2-chloro-4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-5-methoxybenzamide (69.7 mg, 71.85 μmol, 49.46% yield, 99% purity) as a yellow solid. MS(M+H)⁺=960.3

¹H NMR (400 MHz, DMSO-d₆) δ=11.08 (br s, 1H), 8.45-8.36 (m, 1H), 8.35-8.22 (m, 2H), 7.98 (s, 1H), 7.70 (dd, J=7.3, 8.3 Hz, 1H), 7.34 (t, J=7.4 Hz, 2H), 7.05 (s, 1H), 5.09 (dd, J=5.3, 12.9 Hz, 1H), 4.90-4.76 (m, 1H), 4.26 (br d, J=13.3 Hz, 1H), 4.06 (br t, J=13.9 Hz, 2H), 4.00-3.91 (m, 1H), 3.91 (s, 4H), 3.32 (br s, 3H), 3.30-3.25 (m, 4H), 3.22-3.13 (m, 1H), 2.93-2.75 (m, 2H), 2.63-2.51 (m, 10H), 2.08-1.94 (m, 3H), 1.93-1.86 (m, 1H), 1.84-1.75 (m, 1H), 1.75-1.66 (m, 2H), 1.63-1.50 (m, 4H), 1.51-1.29 (m, 2H).

### Example 79. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((1-(2-(2,6-dioxopiperidin-3-yl)-1,3-dioxoisoindolin-4-yl)piperidin-4-yl)oxy)acetyl)piperidin-4-yl)-3-methoxybenzamide (Compound 79)

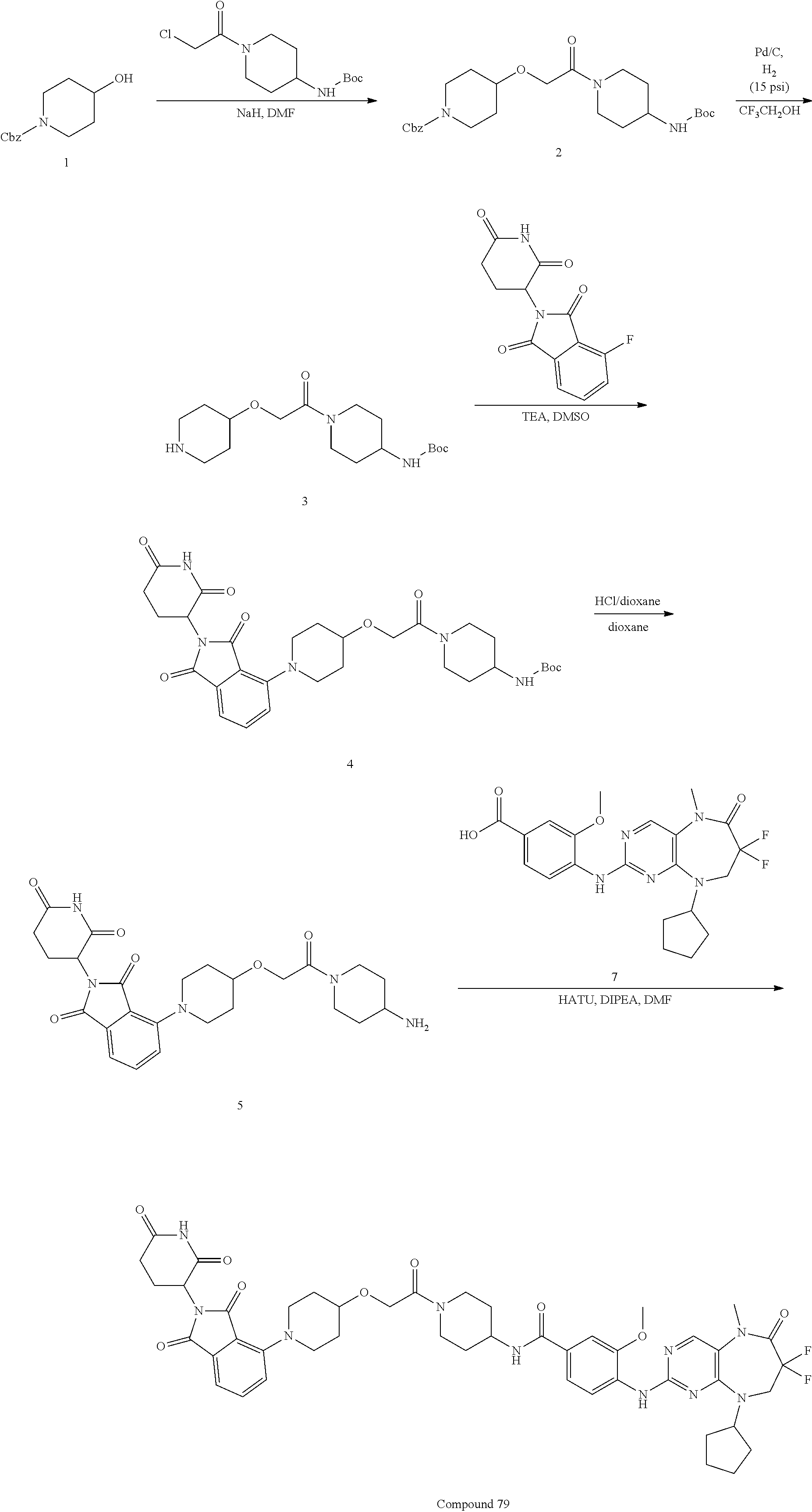

### Step 1. Synthesis of benzyl 4-(2-(4-((tert-butoxycarbonyl)amino)piperidin-1-yl)-2-oxoethoxy) piperidine-1-carboxylate (2)

To a solution of benzyl 4-hydroxypiperidine-1-carboxylate (200 mg, 850.06 μmol) in DMF (10 mL) was added NaH (51.00 mg, 1.28 mmol, 60% purity) at 0° C. after stirring for 0.5 h, then tert-butyl (1-(2-chloroacetyl)piperidin-4-yl)carbamate (258.79 mg, 935.06 μmol) was added and the resulting mixture was stirred at 25° C. for 16 hr. LCMS showed desired product. The mixture was diluted with brine (10 mL), extracted with EtOAc (5 mL×3), the organic layer was dried over Na₂SO₄, filtered and concentrated in vacuum. The crude product was purified by flash silica gel chromatography (Biotage, 4 g SepaFlash® Silica Flash Column, Eluent of 4˜100% EtOAc/Petroleum ether gradient @20 mL/min) to afford benzyl 4-(2-(4-((tert-butoxycarbonyl)amino)piperidin-1-yl-2-oxoethoxy)piperidine-1-carboxylate (210 mg, 362.09 μmol, 42.60% yield, 82% purity) as yellow oil. MS(M+H)⁺=476.1

### Step 2. Synthesis of tert-butyl (1-(2-(piperidin-4-yloxy)acetyl)piperidin-4-yl)carbamate (3)

To a solution of benzyl 4-(2-(4-((tert-butoxycarbonyl)amino)piperidin-1-yl)-2-oxoethoxy)piperidine-1-carboxylate (160 mg, 336.43 μmol) in CF₃CH₂OH (3 mL) was added Pd/C (0.16 g, 33.64 μmol, 10% purity) under N₂, then H₂ (0.16 g, 79.37 mmol) was bubbled into the solution, the mixture was stirred at 25° C. for 2 hr under H₂ (15 psi). LCMS showed desired mass. The mixture was filtered. The filtrate was concentrated in vacuum to afford tert-butyl (1-(2-(piperidin-4-yloxy)acetyl)piperidin-4-yl)carbamate (180 mg, 437.55 μmol, 130.06% yield, 83% purity) as yellow oil. MS(M+H)⁺=342.1

### Step 3. Synthesis of tert-butyl (1-(2-((1-(2-(2,6-dioxopiperidin-3-yl)-1,3-dioxoisoindolin-4-yl)piperidin-4-yl)oxy)acetyl)piperidin-4-yl)carbamate (4)

To a solution of tert-butyl (1-(2-(piperidin-4-yloxy)acetyl)piperidin-4-yl)carbamate (130 mg, 380.74 μmol) in DMSO (2 mL) were added TEA (115.58 mg, 1.14 mmol, 158.98 μL) and 2-(2,6-dioxopiperidin-3-yl)-4-fluoroisoindoline-1,3-dione (105.17 mg, 380.74 μmol), the mixture was stirred at 90° C. for 16 hr. LCMS showed desired mass was detected, TLC (100% EA) showed a main spot was formed. The mixture was diluted with brine (10 mL), extracted with EtOAc (10 mL×3), the organic layer was dried over Na₂SO₄, filtered and concentrated in vacuum. The crude product was purified by flash silica gel chromatography (Biotage, 4 g SepaFlash® Silica Flash Column, Eluent of 4˜100% EtOAc/Petroleum ether gradient @30 mL/min) to afford tert-butyl (1-(2-((1-(2-(2,6-dioxopiperidin-3-yl)-1,3-dioxoisoindolin-4-yl)piperidin-4-yl)oxy)acetyl)piperidin-4-yl)carbamate (110 mg, 184.05 μmol, 48.34% yield, 100% purity) as yellow powder. MS(M+H)⁺=598.7

¹H NMR (400 MHz, DMSO-d₆) δ=11.10 (s, 1H), 7.72-7.64 (m, 1H), 7.34 (dd, J=5.7, 7.5 Hz, 2H), 6.89 (br d, J=7.1 Hz, 1H), 5.10 (dd, J=5.3, 13.0 Hz, 1H), 4.25-4.10 (m, 3H), 3.85-3.73 (m, 1H), 3.63-3.48 (m, 4H), 3.12-2.98 (m, 4H), 2.95-2.81 (m, 2H), 2.76-2.64 (m, 2H), 2.63-2.53 (m, 2H), 2.03-2.12 (m, 2H), 1.80-1.62 (m, 4H), 1.38 (s, 9H).

### Step 4. Synthesis of 4-(4-(2-(4-aminopiperidin-1-yl)-2-oxoethoxy)piperidin-1-yl)-2-(2,6-dioxopiperidin-3-yl)isoindoline-1,3-dione (5)

To a solution of tert-butyl (1-(2-((1-(2-(2,6-dioxopiperidin-3-yl)-1,3-dioxoisoindolin-4-yl)piperidin-4-yl)oxy)acetyl)piperidin-4-yl)carbamate (50 mg, 83.66 μmol) in dioxane (1 mL), was added HCl/dioxane (4 M, 20.91 μL), the mixture was stirred at 25° C. for 1 hr. LCMS showed desired mass. The mixture was concentrated under vacuum to afford 4-(4-(2-(4-aminopiperidin-1-yl)-2-oxoethoxy)piperidin-1-yl)-2-(2,6-dioxopiperidin-3-yl)isoindoline-1,3-dione (46 mg, 80.11 μmol, 95.76% yield, 93% purity, HCl salt) as yellow powder, which was used for the next step directly. MS(M+H)⁺=498.1

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((1-(2-(2,6-dioxopiperidin-3-yl)-1,3-dioxoisoindolin-4-yl)piperidin-4-yl)oxy)acetyl)piperidin-4-yl)-3-methoxybenzamide (Compound 79)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (50 mg, 111.75 μmol) in DMF (1 mL) were added DIPEA (43.33 mg, 335.24 μmol, 58.39 μL) and HATU (63.74 mg, 167.62 μmol), after stirring at 25° C. for 0.5 h, then 4-(4-(2-(4-aminopiperidin-1-yl)-2-oxoethoxy)piperidin-1-yl)-2-(2,6-dioxopiperidin-3-yl)isoindoline-1,3-dione (41.77 mg, 78.22 μmol, HCl salt) was added and the resulting mixture was stirred at 25° C. for 16 h. LCMS showed desired mass. The mixture was purified by Prep-HPLC (column: Phenomenex Synergi Polar-RP 100*25 mm*4 μm; mobile phase: [water(TFA)-ACN]; B %: 44%-64%, 7 min) and Prep-HPLC (column: Waters Xbridge 150*25 mm*5 μm; mobile phase: [water(NH₄HCO 3)-ACN]; B %: 40%-70%) followed by lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((1-(2-(2,6-dioxopiperidin-3-yl)-1,3-dioxoisoindolin-4-yl)piperidin-4-yl)oxy)acetyl)piperidin-4-yl)-3-methoxybenzamide (28.3 mg, 30.04 μmol, 26.88% yield, 98.4% purity) as yellow powder. MS(M+H)⁺=927.1

¹H NMR (400 MHz, DMSO-d₆) δ=11.17-10.94 (m, 1H), 8.31-8.25 (m, 2H), 8.18 (d, J=7.6 Hz, 1H), 7.97 (s, 1H), 7.69 (t, J=7.7 Hz, 1H), 7.52-7.46 (m, 2H), 7.35 (t, J=7.7 Hz, 2H), 5.10 (dd, J=5.4, 12.7 Hz, 1H), 4.77 (t, J=7.9 Hz, 1H), 4.35 (d, J=12.7 Hz, 1H), 4.30-4.15 (m, 2H), 4.05 (t, J=14.0 Hz, 3H), 3.96-3.85 (m, 4H), 3.64-3.50 (m, 3H), 3.32 (s, 3H), 3.18-3.03 (m, 3H), 2.96-2.81 (m, 1H), 2.78-2.65 (m, 1H), 2.64-2.54 (m, 2H), 2.09-1.78 (m, 7H), 1.77-1.50 (m, 9H), 1.49-1.36 (m, 1H).

### Example 80. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((1-(2-(2,6-dioxopiperidin-3-yl)-1,3-dioxoisoindolin-4-yl)azetidin-3-yl)oxy)acetyl)piperidin-4-yl)-3-methoxybenzamide (Compound 80)

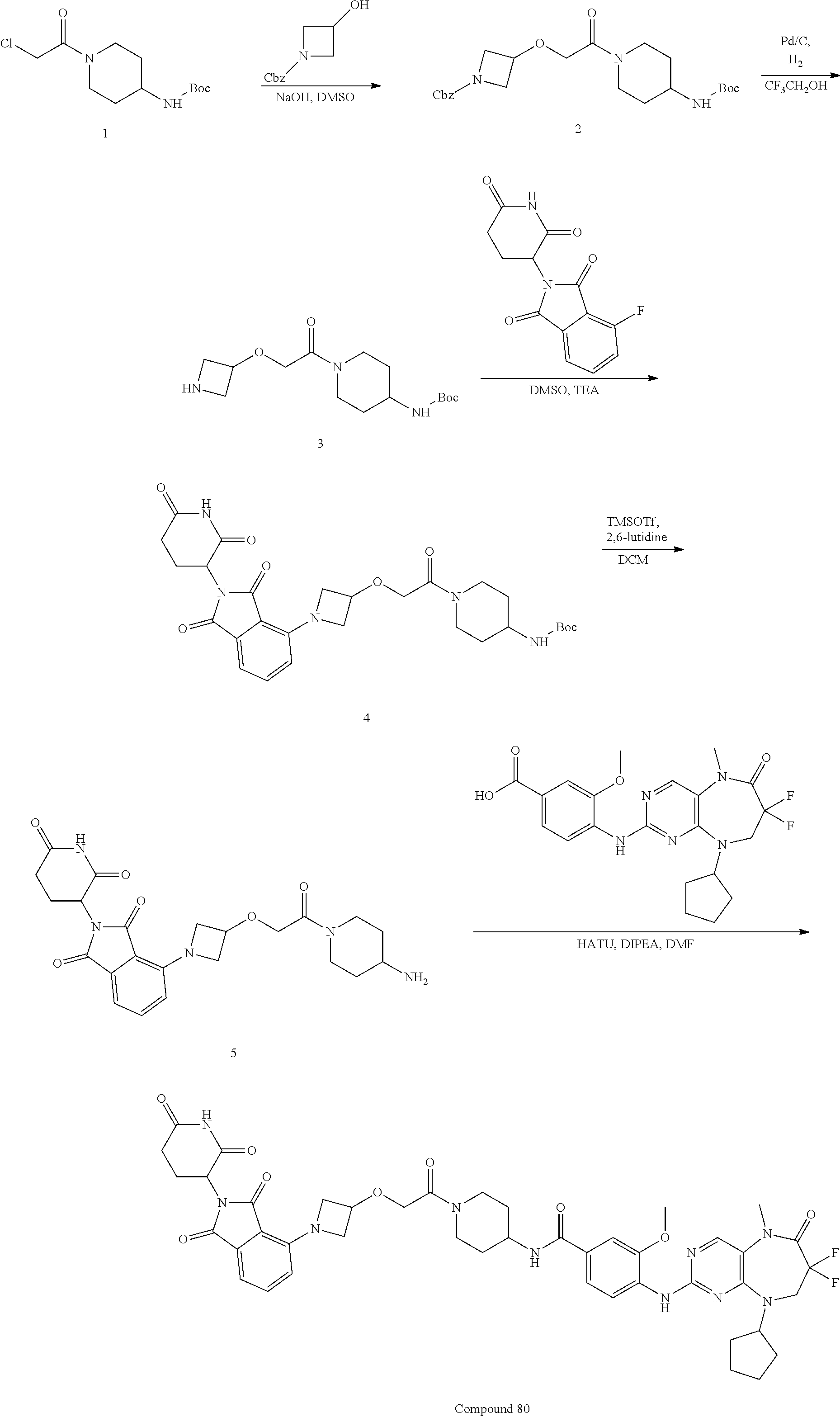

### Step 1. Synthesis of benzyl 3-(2-(4-((tert-butoxycarbonyl)amino)piperidin-1-yl)-2-oxoethoxy)azetidine-1-carboxylate (2)

To a solution of benzyl 3-hydroxyazetidine-1-carboxylate (300 mg, 1.45 mmol) in DMSO (8 mL) was added the solution of NaOH (98.44 mg, 2.46 mmol) in Water (5 mL) at 20° C. under N₂, then tert-butyl (1-(2-chloroacetyl)piperidin-4-yl)carbamate (480.80 mg, 1.74 mmol) was added, the mixture was stirred at 20° C. for 16 h. LCMS showed desired mass. The mixture was diluted with brine (10 mL), extracted with EtOAc (10 mL×3), the organic layer was dried over Na₂SO₄, filtered and concentrated under vacuum. The crude product was purified by flash silica gel chromatography (Biotage, 4 g SepaFlash® Silica Flash Column, Eluent of 1˜100% EtOAc/Petroleum ether gradient @30 mL/min) to afford benzyl 3-(2-(4-((tert-butoxycarbonyl)amino)piperidin-1-yl)-2-oxoethoxy)azetidine-1-carboxylate (350 mg, 649.13 μmol, 44.84% yield, 83% purity) as a white solid. MS(M+H)⁺=448.1

### Step 2. Synthesis of tert-butyl (1-(2-(azetidin-3-yloxy)acetyl)piperidin-4-yl)carbamate (3)

To a solution of benzyl 3-(2-(4-((tert-butoxycarbonyl)amino)piperidin-1-yl)-2-oxoethoxy)azetidine-1-carboxylate (250 mg, 558.63 μmol) in CF₃CH₂OH (6 mL) was added Pd/C (200 mg, 187.93 μmol, 10% purity) under N₂, the mixture was stirred under 15 psi of H₂ at 25° C. for 2 hr. LCMS showed desired mass. The mixture was filtered to obtain the filter liquor, which was concentrated under vacuum to afford tert-butyl (1-(2-(azetidin-3-yloxy)acetyl)piperidin-4-yl)carbamate (190 mg, 297.07 μmol, 53.18% yield, 49% purity) as yellow oil, used directly. MS(M+H)⁺=314.0

### Step 3. Synthesis of tert-butyl (1-(2-((1-(2-(2,6-dioxopiperidin-3-yl)-1,3-dioxoisoindolin-4-yl)azetidin-3-yl)oxy)acetyl)piperidin-4-yl)carbamate (4)

To a solution of tert-butyl (1-(2-(azetidin-3-yloxy)acetyl)piperidin-4-yl)carbamate (190 mg, 606.27 μmol) in DMSO (10 mL) were added TEA (184.04 mg, 1.82 mmol, 253.16 μL) and 2-(2,6-dioxopiperidin-3-yl)-4-fluoroisoindoline-1,3-dione (167.46 mg, 606.27 μmol), the mixture was stirred at 90° C. for 16 hr. LCMS showed desired product, TLC (EA) showed a main spot. The mixture was diluted with brine (10 mL), extracted with EtOAc (10 mL×3), the organic layer was dried over Na₂SO₄, filtered and concentrated under vacuum. The crude product was purified by flash silica gel chromatography (Biotage, 4 g SepaFlash® Silica Flash Column, Eluent of 4˜100% EtOAc/Petroleum ether gradient @30 mL/min) to afford tert-butyl (1-(2-((1-(2-(2,6-dioxopiperidin-3-yl)-1,3-dioxoisoindolin-4-yl)azetidin-3-yl)oxy)acetyl)piperidin-4-yl)carbamate (224 mg, 334.27 μmol, 55.13% yield, 85% purity) as yellow powder. MS(M+H)⁺=570.1

¹H NMR (400 MHz, DMSO-d₆) δ=7.58 (t, J=7.8 Hz, 1H), 7.14 (d, J=6.8 Hz, 1H), 6.88 (d, J=7.5 Hz, 1H), 6.81 (d, J=8.4 Hz, 1H), 5.06 (dd, J=5.4, 12.8 Hz, 1H), 4.47-4.36 (m, 3H), 4.28-4.14 (m, 3H), 4.03 (q, J=7.0 Hz, 2H), 3.73-3.65 (m, 1H), 3.54-3.42 (m, 1H), 3.34-3.30 (m, 2H), 3.08-2.96 (m, 1H), 2.94-2.82 (m, 1H), 2.77-2.66 (m, 1H), 2.63-2.56 (m, 1H), 1.99 (s, 2H), 1.79-1.65 (m, 2H), 1.41-1.37 (m, 9H)

### Step 4. Synthesis of 4-(3-(2-(4-aminopiperidin-1-yl)-2-oxoethoxy)azetidin-1-yl)-2-(2,6-dioxopiperidin-3-yl)isoindoline-1,3-dione (5)

To a solution of tert-butyl (1-(2-((1-(2-(2,6-dioxopiperidin-3-yl)-1,3-dioxoisoindolin-4-yl)azetidin-3-yl)oxy)acetyl)piperidin-4-yl)carbamate (90 mg, 158.00 μmol) in DCM (4 mL) was added 2,6-LUTIDINE (414.00 mg, 3.86 mmol, 450.00 μL), then TMSOTf (553.50 mg, 2.49 mmol, 450.00 μL) was added, the mixture was stirred at 25° C. for 2 h. LCMS showed desired product. The mixture was diluted with water (2 mL), extracted with EtOAc (5 mL×3), the aqueous layer was concentrated by freeze drying to obtained the crude product. The crude product was purified by Prep-HPLC (column: Waters Xbridge 150*25 mm*5 μm; mobile phase: [water(NH₄HCO₃)-ACN]; B %: 10%-40%, min) and followed by lyophilization to afford 4-(3-(2-(4-aminopiperidin-1-yl)-2-oxoethoxy)azetidin-1-yl)-2-(2,6-dioxopiperidin-3-yl)isoindoline-1,3-dione (20 mg, 40.47 μmol, 25.61% yield, 95% purity) as yellow powder. MS(M+H)⁺=470.0

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((1-(2-(2,6-dioxopiperidin-3-yl)-1,3-dioxoisoindolin-4-yl)azetidin-3-yl)oxy)acetyl)piperidin-4-yl)-3-methoxybenzamide (Compound 80)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (20 mg, 44.70 μmol) in DMF (1 mL) were added HATU (17.00 mg, 44.70 μmol) and DIEA (17.33 mg, 134.10 μmol, 23.36 μL) and the mixture was stirred at 25° C. for 0.5 h, then 4-(3-(2-(4-aminopiperidin-1-yl)-2-oxoethoxy)azetidin-1-yl)-2-(2,6-dioxopiperidin-3-yl)isoindoline-1,3-dione (19.94 mg, 42.46 μmol) was added, the mixture was stirred at 25° C. for 16 h. LCMS showed the main peak with desired mass. The mixture was diluted with water (2 mL), extracted with EtOAc (2 mL×3), the organic layer was dried over Na₂SO₄, filtered and concentrated under vacuum. The crude product was purified by prep-TLC (DCM:MeOH=10:1) and prep-HPLC (column: Waters Xbridge 150*25 mm*5 μm; mobile phase: [water(NH₄HCO₃)-ACN]; B %: 38%-68%, min) followed by freeze drying to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((1-(2-(2,6-dioxopiperidin-3-yl)-1,3-dioxoisoindolin-4-yl)azetidin-3-yl)oxy)acetyl)piperidin-4-yl)-3-methoxybenzamide (18.2 mg, 19.56 μmol, 43.76% yield, 96.6% purity) as yellow powder. MS(M+H)⁺=899.0

¹H NMR (400 MHz, DMSO-d₆) δ=11.28-10.90 (m, 1H), 8.34-8.24 (m, 2H), 8.18 (d, J=7.8 Hz, 1H), 7.98 (s, 1H), 7.59 (t, J=7.7 Hz, 1H), 7.54-7.45 (m, 2H), 7.15 (d, J=7.1 Hz, 1H), 6.83 (d, J=8.4 Hz, 1H), 5.06 (dd, J=5.3, 12.8 Hz, 1H), 4.83-4.71 (m, 1H), 4.52-4.17 (m, 7H), 4.11-4.00 (m, 5H), 3.94 (s, 3H), 3.85-3.77 (m, 1H), 3.33 (s, 3H), 3.17-3.04 (m, 1H), 2.60-2.82 (m, 4H), 2.04-1.82 (m, 5H), 1.71-1.44 (m, 7H).

### Example 81. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1-oxoisoindolin-4-yl)piperazin-1-yl)propanoyl)piperidin-4-yl)-3-methoxybenzamide (Compound 81)

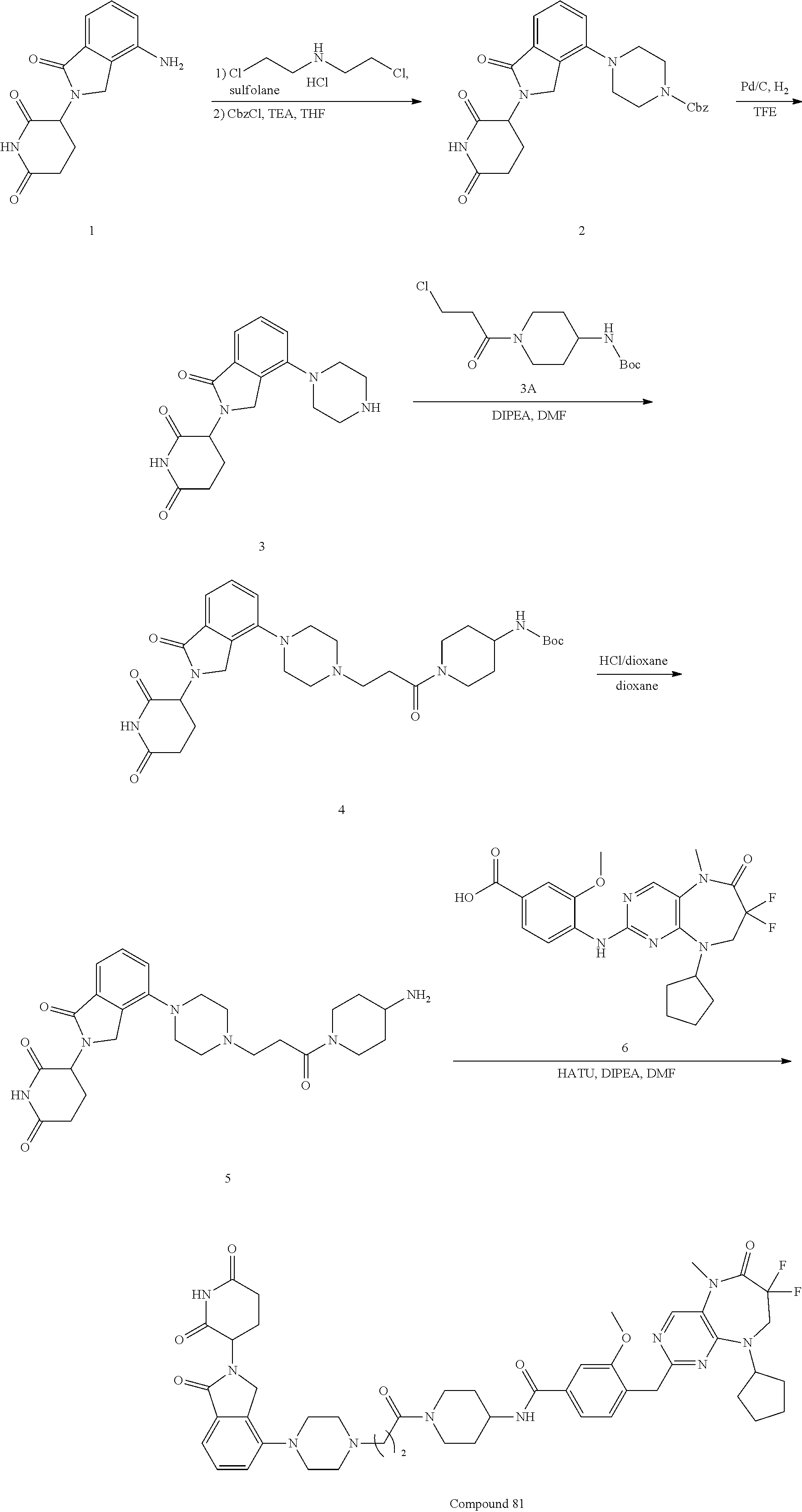

### Step 1. Synthesis of benzyl 4-(2-(2,6-dioxopiperidin-3-yl)-1-oxoisoindolin-4-yl)piperazine-1-carboxylate (2)

A solution of 3-(4-amino-1-oxoisoindolin-2-yl)piperidine-2,6-dione (10 g, 38.57 mmol) and bis(2-chloroethyl)amine (5.48 g, 30.69 mmol, HCl) in thiolane 1,1-dioxide (60 mL) was heated at 140° C. for 12 h. LCMS showed a peak (48%) of reactant remained, the mixture was heated at 140° C. for another 12 h. LCMS showed a peak (45%) with desired mass. To the mixture were added TEA (11.71 g, 115.71 mmol, 16.11 mL) and a solution CbzCl (6.58 g, 38.57 mmol, 5.48 mL) in THF (20 mL) and it was stirred at 25° C. for another 1 h. LCMS showed a peak (68%) with desired mass. The reaction mixture was slowly poured into the H₂O (300 mL). The mixture was filtered, The filtrate was extracted with EtOAc (400 mL×2). The combined organic layers were dried over Na₂SO₄ and combined with filter cake and were concentrated under reduced pressure to give a residue. The residue were purified by silica gel column chromatography (EtOAc:DCM=10:1) to afford benzyl 4-(2-(2,6-dioxopiperidin-3-yl)-1-oxoisoindolin-4-yl)piperazine-1-carboxylate (3.1 g, 6.70 mmol, 17.38% yield) as yellow solid. MS(M+H)⁺=463.1

### Step 2. Synthesis of 3-(1-oxo-4-(piperazin-1-yl)isoindolin-2-yl)piperidine-2,6-dione (3)

To a solution of benzyl 4-(2-(2,6-dioxopiperidin-3-yl)-1-oxoisoindolin-4-yl)piperazine-1-carboxylate (2.5 g, 5.41 mmol) in TFE (20 mL) was added Pd/C (200 mg, 324.33 μmol, 10% purity) under N₂. The suspension was degassed under vacuum and purged with H₂ several times. The mixture was stirred under H₂ (15 psi) at 20° C. for 12 h. LCMS showed a peak (68%) of reactant remained, the mixture was stirred at 20° C. for another 12 h. LCMS showed a peak (89%) with desired mass. The mixture was filtered, the filtrate was concentrated in vacuo to afford 3-(1-oxo-4-(piperazin-1-yl)isoindolin-2-yl)piperidine-2,6-dione (2.1 g, crude) as yellow solid. MS(M+H)⁺=329.1

### Step 3. Synthesis of tert-butyl (1-(3-(4-(2-(2,6-dioxopiperidin-3-yl)-1-oxoisoindolin-4-yl)piperazin-1-yl)propanoyl)piperidin-4-yl)carbamate (4)

To a solution of 3-(1-oxo-4-(piperazin-1-yl)isoindolin-2-yl)piperidine-2,6-dione (300 mg, 913.62 μmol) and tert-butyl (1-(3-chloropropanoyl)piperidin-4-yl)carbamate (1.06 g, 3.65 mmol) in DMF (5 mL) was added DIPEA (354.24 mg, 2.74 mmol, 477.41 μL), the mixture was heated at 70° C. for 24 h. LCMS showed a peak (36%) with desired mass. The mixture was concentrated in vacuo. The residue was purified by flash silica gel chromatography (Biotage, 12 g SepaFlash® Silica Flash Column, Eluent of 10˜30% EtOAc/MeOH gradient @60 mL/min) to afford tert-butyl (1-(3-(4-(2-(2,6-dioxopiperidin-3-yl)-1-oxoisoindolin-4-yl)piperazin-1-yl)propanoyl)piperidin-4-yl)carbamate (170 mg, 291.75 μmol, 31.93% yield) as white solid. MS(M+H)⁺=583.2

### Step 4. Synthesis of 3-(4-(4-(3-(4-aminopiperidin-1-yl)-3-oxopropyl)piperazin-1-yl)-1-oxoisoindolin-2-yl)piperidine-2,6-dione (5)

A solution of tert-butyl N-[1-[3-[4-[2-(2,6-dioxo-3-piperidyl)-1-oxo-isoindolin-4-yl]piperazin-1-yl]propanoyl]-4-piperidyl]carbamate (170 mg, 291.75 μmol) and HCl/dioxane (4 M, 2 mL) in dioxane (2 mL) was stirred at 20° C. for 1 h. LCMS showed a peak (61%) with desired mass. The mixture was concentrated in vacuo to afford 3-(4-(4-(3-(4-aminopiperidin-1-yl)-3-oxopropyl)piperazin-1-yl)-1-oxoisoindolin-2-yl)piperidine-2,6-dione (150 mg, crude, HCl) was obtained as white solid. MS(M+H)⁺=483.2

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1-oxoisoindolin-4-yl)piperazin-1-yl)propanoyl)piperidin-4-yl)-3-methoxybenzamide (Compound 81)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (68.96 mg, 154.13 μmol) in DMF (1.5 mL) were added HATU (117.21 mg, 308.26 μmol) and DIPEA (59.76 mg, 462.40 μmol, 80.54 μL), the mixture was stirred at 25° C. for 10 min, then 3-(4-(4-(3-(4-aminopiperidin-1-yl)-3-oxopropyl)piperazin-1-yl)-1-oxoisoindolin-2-yl)piperidine-2,6-dione (80 mg, 154.13 μmol, HCl) was added, the mixture was stirred at 25° C. for another 1 h. LCMS showed a peak (48%) with desired mass. The reaction mixture was diluted with H₂O (8 mL) and extracted with EtOAc (10 mL×3). The combined organic layers were dried over Na₂SO₄, filtered and concentrated under reduced pressure to give a residue. The residue was purified by prep-HPLC (column: Waters Xbridge 150*25 mm*5 μm; mobile phase: [water (10 mM NH₄HCO₃)-ACN]; B %: 33%-63%, min) followed by lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1-oxoisoindolin-4-yl)piperazin-1-yl)propanoyl)piperidin-4-yl)-3-methoxybenzamide (26.1 mg, 26.59 μmol, 17.25% yield, 92.9% purity) as white solid. MS(M+H)⁺=912.3

¹H NMR (400 MHz, DMSO-d₆) δ=10.98 (s, 1H), 8.30-8.24 (m, 2H), 8.15 (d, J=7.8 Hz, 1H), 7.97 (s, 1H), 7.51-7.41 (m, 3H), 7.31 (d, J=7.3 Hz, 1H), 7.16 (d, J=7.9 Hz, 1H), 5.15-5.08 (m, 1H), 4.81-4.71 (m, 1H), 4.47-4.28 (m, 3H), 4.08-4.00 (m, 3H), 3.95-3.92 (m, 4H), 3.31-3.31 (m, 3H), 3.19-3.01 (m, 6H), 2.97-2.87 (m, 1H), 2.64-2.55 (m, 10H), 2.02-1.88 (m, 4H), 1.86-1.80 (m, 1H), 1.72-1.69 (m, 2H), 1.69-1.51 (m, 4H), 1.51-1.36 (m, 2H)

### Example 82. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1-oxoisoindolin-5-yl)piperazin-1-yl)propanoyl)piperidin-4-yl)-3-methoxybenzamide (Compound 82)

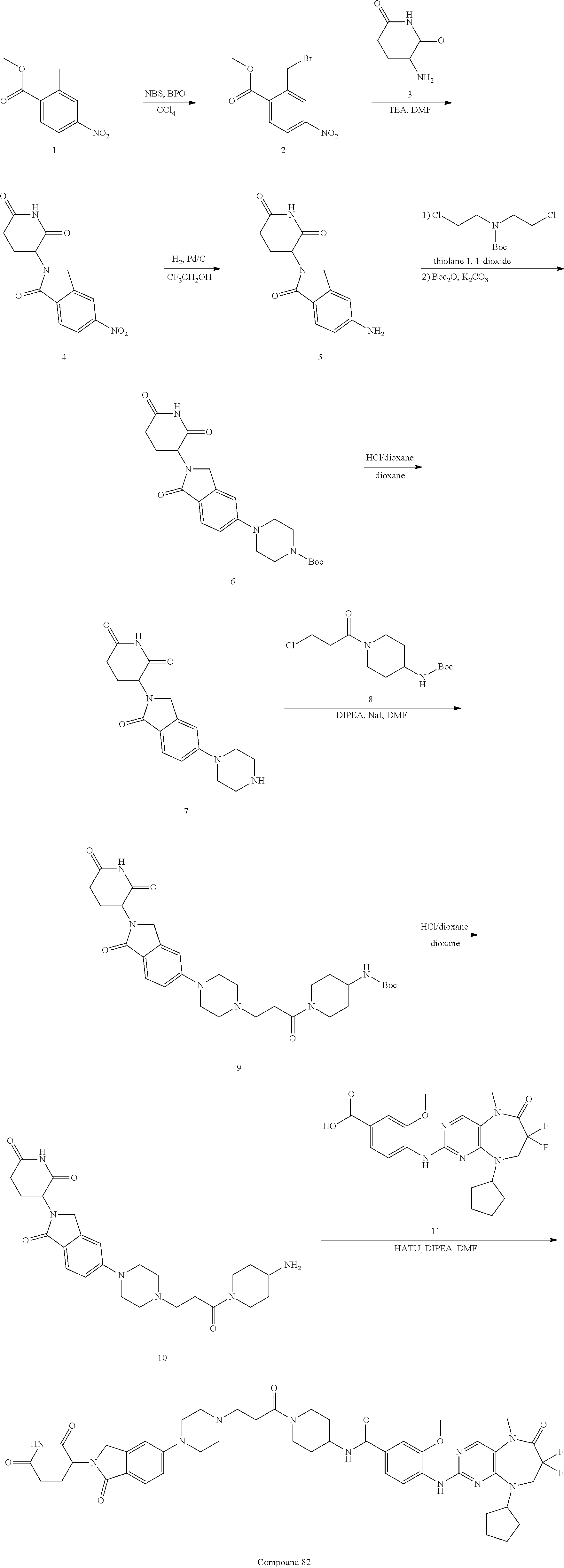

### Step 1. Synthesis of methyl 2-(bromomethyl)-4-nitrobenzoate (2)

To a solution of methyl 2-methyl-4-nitrobenzoate (10 g, 51.24 mmol) in CCl₄ (200 mL) was added NBS (10.93 g, 61.43 mmol) and BPO (266.67 mg, 1.10 mmol). The reaction mixture was stirred at 85° C. for 12 hrs. TLC (Petroleum ether:EtOAc=10:1) indicated the starting material was consumed completely and one new spot with larger polarity was formed. The mixture was washed with saturated NaHCO₃ (60 mL) and brine (150.0 mL). The organic layer was dried over anhydrous NaSO₄, filtered and concentrated in vacuo. The residue was purified by flash silica gel chromatography (Biotage; 80 g SepaFlash® Silica Flash Column, Eluent of 0˜2% EtOAc/Petroleum ether gradient @100 mL/min) to afford methyl 2-(bromomethyl)-4-nitrobenzoate (8.5 g, 31.01 mmol, 60.53% yield) as a white solid. MS(M+H)⁺=275.1

### Step 2. Synthesis of 3-(5-nitro-1-oxoisoindolin-2-yl)piperidine-2,6-dione (4)

To a solution of methyl 2-(bromomethyl)-4-nitrobenzoate (2 g, 7.30 mmol) in DMF (20 mL) were added 3-aminopiperidine-2,6-dione (935.02 mg, 7.30 mmol) and TEA (2.22 g, 21.89 mmol, 3.05 mL), the resulting mixture was stirred at 75° C. for 16 h. LCMS showed a main peak with desired mass. The reaction mixture was diluted with brine (60 mL), extracted with EtOAc (60 mL×5). The combined organic layers were washed with brine (100 mL×2), dried over Na₂SO₄, filtered and concentrated in vacuo. The residue was triturated with EtOAc (10 mL) at 20° C. for 10 min to afford 3-(5-nitro-1-oxoisoindolin-2-yl)piperidine-2,6-dione (1.2 g, 3.69 mmol, 50.60% yield, 89% purity) as a blue solid. MS(M+H)⁺=290.0

### Step 3. Synthesis of 3-(5-amino-1-oxoisoindolin-2-yl)piperidine-2,6-dione (5)

A mixture of 3-(5-nitro-1-oxoisoindolin-2-yl)piperidine-2,6-dione (1.2 g, 4.15 mmol) and Pd/C (100 mg, 10% purity) in CF₃CH₂OH (120 mL) was degassed and purged with H₂ for 3 times, the resulting mixture was stirred at 20° C. for 6 h under H₂ (15 Psi) atmosphere. LCMS showed a main peak with desired mass. The suspension was filtered through a pad of celite, the filter cake was washed with CF₃CH₂OH (100 mL). The combined filtrates were concentrated to dryness to give a residue. The residue was triturated with EtOAc (20 mL) at 20° C. for 10 min to afford 3-(5-amino-1-oxoisoindolin-2-yl)piperidine-2,6-dione (900 mg, crude) as a blue solid. MS(M+H)⁺=260.0

### Step 4. Synthesis of tert-butyl 4-(2-(2,6-dioxopiperidin-3-yl)-1-oxoisoindolin-5-yl)piperazine-1-carboxylate (6)

A mixture of 3-(5-amino-1-oxoisoindolin-2-yl)piperidine-2,6-dione (500 mg, 1.93 mmol) and tert-butyl bis(2-chloroethyl)carbamate (466.99 mg, 1.93 mmol) in thiolane 1,1-dioxide (10 mL) was stirred at 140° C. for 16 h. LCMS showed 63% of 3-(5-amino-1-oxoisoindolin-2-yl)piperidine-2,6-dione remained and a peak (16%) with mass of de-Boc product. Additional tert-butyl bis(2-chloroethyl)carbamate (500 mg) was added and the mixture was stirred at 140° C. for another 24 h. LCMS showed trace of 3-(5-amino-1-oxoisoindolin-2-yl)piperidine-2,6-dione remained and a peak (66%) with mass of de-Boc product. The mixture was adjusted to pH=9 using K₂CO₃ (aq., 2 M), Boc₂O (505.08 mg, 2.31 mmol, 531.67 μL) was added and the resulting mixture was stirred at 20° C. for 2 h. LCMS showed a peak (81%) with desired mass. The reaction mixture was diluted with brine (20 mL), extracted with EtOAc (20 mL×3). The combined organic layers were washed with brine (20 mL×2), dried over Na₂SO₄, filtered. The filtrate was concentrated under reduced pressure. The residue was purified by flash silica gel chromatography (Biotage; 80 g SepaFlash® Silica Flash Column, Eluent of 20˜100% EtOAc/Petroleum ether gradient @100 mL/min) followed by triturated with MTBE (10 mL) at 25° C. for 10 min to afford tert-butyl 4-(2-(2,6-dioxopiperidin-3-yl)-1-oxoisoindolin-5-yl)piperazine-1-carboxylate (450 mg, 1.05 mmol, 54.46% yield, 100% purity) as a yellow solid. MS(M+H)⁺=429.0

### Step 5. Synthesis of 3-(1-oxo-5-(piperazin-1-yl)isoindolin-2-yl)piperidine-2,6-dione (7)

To a solution of tert-butyl 4-(2-(2,6-dioxopiperidin-3-yl)-1-oxoisoindolin-5-yl)piperazine-1-carboxylate (200 mg, 466.77 μmol) in dioxane (4 mL) was added HCl/dioxane (4 M, 4 mL) and the mixture was stirred at 20° C. for 1 h. LCMS showed a peak (98%) with desired mass. The mixture was concentrated under reduced pressure to afford 3-(1-oxo-5-(piperazin-1-yl)isoindolin-2-yl)piperidine-2,6-dione (170 mg, crude, HCl salt) as a yellow solid. MS(M+H)⁺=329.0

### Step 6. Synthesis of tert-butyl (1-(3-(4-(2-(2,6-dioxopiperidin-3-yl)-1-oxoisoindolin-5-yl)piperazin-1-yl)propanoyl)piperidin-4-yl)carbamate (9)

To a solution of 3-(1-oxo-5-(piperazin-1-yl)isoindolin-2-yl)piperidine-2,6-dione (150 mg, 411.16 μmol, HCl salt) in DMF (5 mL) were added tert-butyl (1-(3-chloropropanoyl)piperidin-4-yl)carbamate (179.34 mg, 616.73 μmol), DIPEA (265.69 mg, 2.06 mmol, 358.08 μL) and NaI (6.75 mg, 45.03 μmol), the resulting mixture was stirred at 60° C. for 16 h. LCMS showed 56% of 3-(1-oxo-5-(piperazin-1-yl)isoindolin-2-yl)piperidine-2,6-dione remained and a peak (16%) with desired mass. Additional tert-butyl (1-(3-chloropropanoyl)piperidin-4-yl)carbamate (200 mg) was added and the mixture was stirred at 60° C. for 16 h. LCMS showed the starting material was consumed completely and a peak (38%) with desired mass. The reaction mixture was diluted with brine (15 mL), then extracted with EtOAc (15 mL×3). The combined organic layers were washed with brine (15 mL×2), dried over Na₂SO₄, filtered. The filtrate was concentrated under reduced pressure. The residue was purified by flash silica gel chromatography (Biotage; 20 g SepaFlash® Silica Flash Column, Eluent of 20˜100% EtOAc/Petroleum ether gradient @80 mL/min) to afford tert-butyl (1-(3-(4-(2-(2,6-dioxopiperidin-3-yl)-1-oxoisoindolin-5-yl)piperazin-1-yl)propanoyl)piperidin-4-yl)carbamate (200 mg, 326.07 μmol, 79.31% yield, 95% purity) as a yellow solid. MS(M+H)⁺=583.1

### Step 7. Synthesis of 3-(5-(4-(3-(4-aminopiperidin-1-yl)-3-oxopropyl)piperazin-1-yl)-1-oxoisoindolin-2-yl)piperidine-2,6-dione (10)

To a solution of tert-butyl (1-(3-(4-(2-(2,6-dioxopiperidin-3-yl)-1-oxoisoindolin-5-yl)piperazin-1-yl)propanoyl)piperidin-4-yl)carbamate (200 mg, 343.24 μmol) in dioxane (2 mL) was added HCl/dioxane (4 M, 85.81 μL), the mixture was stirred at 20° C. for 1 h. LCMS showed a peak (97%) with desired mass. The mixture was concentrated under reduced pressure to afford 3-(5-(4-(3-(4-aminopiperidin-1-yl)-3-oxopropyl)piperazin-1-yl)-1-oxoisoindolin-2-yl)piperidine-2,6-dione (160 mg, crude) as a yellow solid, which was used into the next step directly. MS(M+H)⁺=483.3

### Step 8. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1-oxoisoindolin-5-yl)piperazin-1-yl)propanoyl)piperidin-4-yl)-3-methoxybenzamide (Compound 82)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (74.17 mg, 165.78 μmol) in DMF (2 mL) were added HATU (94.55 mg, 248.67 μmol), DIPEA (64.28 mg, 497.33 μmol, 86.63 μL), the mixture was stirred at 20° C. for 0.5 h, then 3-(5-(4-(3-(4-aminopiperidin-1-yl)-3-oxopropyl)piperazin-1-yl)-1-oxoisoindolin-2-yl)piperidine-2,6-dione (80 mg, 165.78 μmol) was added and the resulting mixture was stirred at 20° C. for 16 h. LCMS showed a peak (48%) with desired mass. The reaction mixture was diluted with brine (10 mL), then extracted with EtOAc (10 mL×3). The combined organic layers were washed with brine (10 mL×2), dried over Na₂SO₄, filtered. The filtrate was concentrated under reduced pressure. The residue was purified by reversed-phase HPLC (column: Phenomenex Synergi C18 150*25 mm*10 μm; mobile phase: [water(FA)-ACN]; B %: 18%-51%, 11 min) and re-purified by reversed-phase HPLC (column: Waters Xbridge 150*25 mm*5 μm; mobile phase: [water(NH₄HCO₃)-ACN]; B %: 30%-60%, 8 min). The eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1-oxoisoindolin-5-yl)piperazin-1-yl)propanoyl)piperidin-4-yl)-3-methoxybenzamide (33.1 mg, 35.21 μmol, 21.24% yield, 97% purity) as a white solid. MS(M+H)⁺=912.1

¹H NMR (400 MHz, DMSO-d₆) δ=10.94 (s, 1H), 8.31-8.24 (m, 2H), 8.15 (d, J=7.8 Hz, 1H), 7.96 (s, 1H), 7.55-7.45 (m, 3H), 7.11-7.03 (m, 2H), 5.09-5.00 (m, 1H), 4.83-4.71 (m, 1H), 4.45-4.15 (m, 3H), 4.12-4.00 (m, 3H), 3.98-3.88 (m, 4H), 3.32-3.25 (m, 8H), 3.18-3.07 (m, 1H), 2.97-2.83 (m, 1H), 2.72-2.56 (m, 9H), 2.40-2.31 (m, 1H), 2.01-1.81 (m, 5H), 1.75-1.66 (m, 2H), 1.64-1.53 (m, 4H), 1.52-1.35 (m, 2H).

### Example 83. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(4-((1-(2-(2,6-dioxopiperidin-3-yl)-1,3-dioxoisoindolin-4-yl)piperidin-4-yl)methyl)piperazin-1-yl)cyclohexyl)-2-fluoro-5-methoxybenzamide (Compound 83)

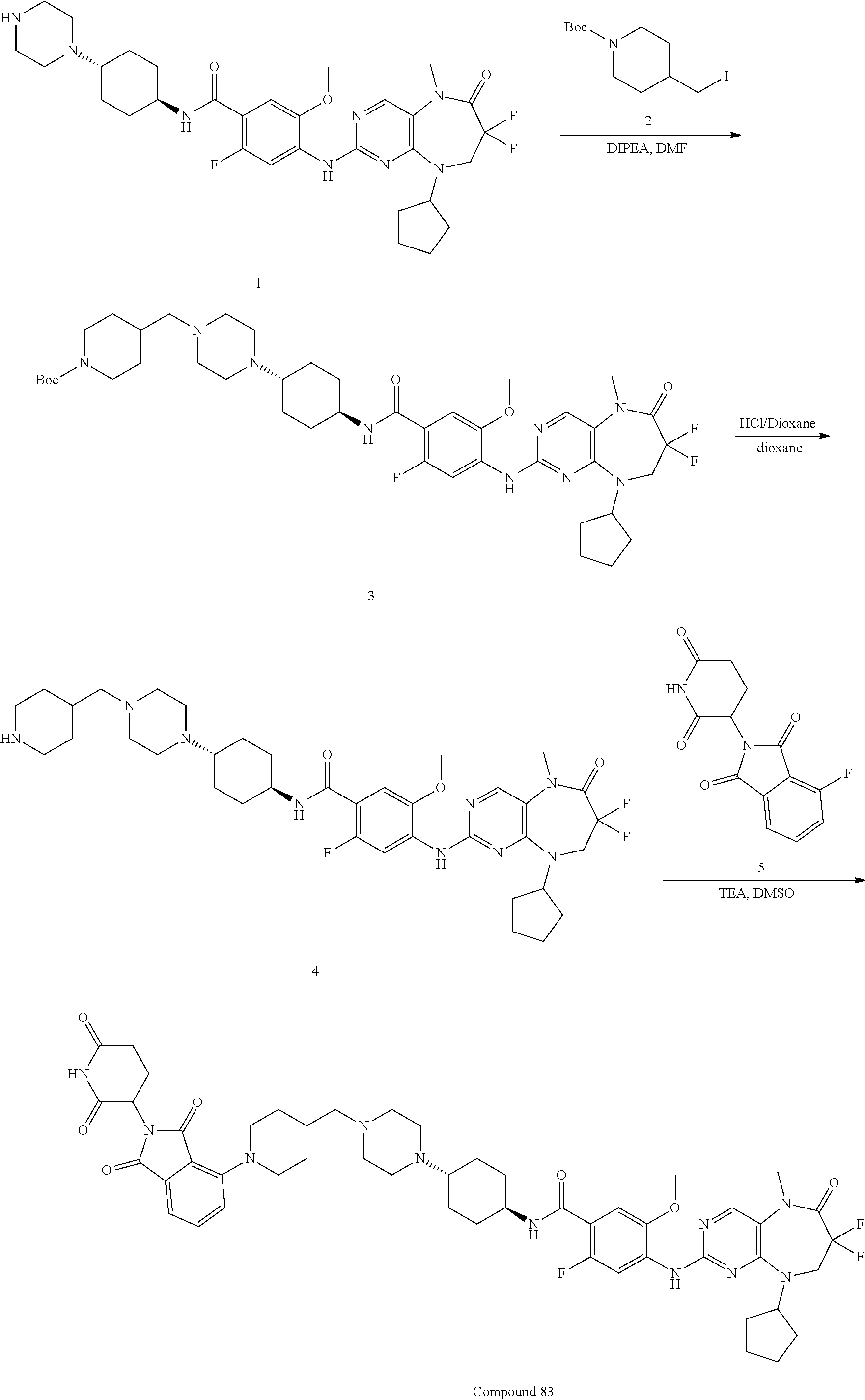

### Step 1. Synthesis of tert-butyl 4-((4-((1r,4r)-4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzamido)cyclohexyl)piperazin-1-yl)methyl)piperidine-1-carboxylate (3)

To a solution of 4-[(9-cyclopentyl-7,7-difluoro-5-methyl-6-oxo-8H-pyrimido[4,5-b][1,4]diazepin-2-yl)amino]-2-fluoro-5-methoxy-N-(4-piperazin-1-ylcyclohexyl) benzamide (150 mg, 237.83 μmol) in DMF (4 mL) were added DIPEA (92.21 mg, 713.49 μmol, 124.28 μL) and tert-butyl 4-(iodomethyl) piperidine-1-carboxylate (85.07 mg, 261.61 μmol) at 20° C. and the resulting mixture was stirred at 60° C. for 16 h. LCMS showed starting material was consumed completely and 67% peak with desired mass was detected. The reaction mixture was diluted with H₂O (12 mL) and extracted with EtOAc (12 mL×3). The organic layer was washed with brine (12 mL×3), dried over Na₂SO₄, filtered and concentrated. The residue was purified by flash silica gel chromatography (4 g SepaFlash® Silica Flash Column, Eluent of 0˜100% EtOAc/Petroleum ether to 0˜10% Dichloromethane/Methanol gradient @60 mL/min) to afford tert-butyl 4-((4-((1r,4r)-4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzamido)cyclohexyl) piperazin-1-yl)methyl)piperidine-1-carboxylate (99 mg, 119.57 μmol, 50.27% yield) as an orange oil. MS(M+H)⁺=828.2

### Step 2. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxy-N-((1r,4r)-4-(4-(piperidin-4-ylmethyl)piperazin-1-yl)cyclohexyl)benzamide (4)

To a solution of tert-butyl 4-((4-((1r,4r)-4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzamido)cyclohexyl) piperazin-1-yl)methyl)piperidine-1-carboxylate (99 mg, 119.57 μmol) in dioxane (1 mL) was added HCl/dioxane (4 M, 6 mL) at 20° C. and the resulting was stirred at 20° C. for 1 h. LCMS showed starting material was consumed completely and 84% peak with desired mass was detected. The reaction mixture was concentrated in vacuum to afford 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxy-N-((1r,4r)-4-(4-(piperidin-4-ylmethyl)piperazin-1-yl)cyclohexyl)benzamide (99 mg, crude, HCl) as an off-white solid. MS(M+H)⁺=728.2

### Step 3. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(4-((1-(2-(2,6-dioxopiperidin-3-yl)-1,3-dioxoisoindolin-4-yl)piperidin-4-yl)methyl)piperazin-1-yl)cyclohexyl)-2-fluoro-5-methoxybenzamide (Compound 83)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxy-N-((1r,4r)-4-(4-(piperidin-4-ylmethyl)piperazin-1-yl)cyclohexyl)benzamide (99 mg, 129.53 μmol, HCl) in DMSO (10 mL) were added TEA (39.32 mg, 388.58 μmol, 54.09 μL) and 2-(2,6-dioxo-3-piperidyl)-4-fluoro-isoindoline-1,3-dione (42.93 mg, 155.43 μmol) at 20° C. and the resulting mixture was stirred at 100° C. for 16 h. LCMS showed 75% of 2-(2,6-dioxo-3-piperidyl)-4-fluoro-isoindoline-1,3-dione remained and 17% peak with desired mass was detected. The reaction mixture was diluted with H₂O (12 mL) and extracted with EtOAc (12 mL×3). The organic layer was dried over Na₂SO₄, filtered and concentrated. The residue was purified by prep-HPLC (column: Unisil 3-100 C₁₈ μLtra 150*50 mm*3 μm; mobile phase: [water(FA)-ACN]; B %: 16%-46%, 15 min) and lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(4-((1-(2-(2,6-dioxopiperidin-3-yl)-1,3-dioxoisoindolin-4-yl)piperidin-4-yl)methyl)piperazin-1-yl)cyclohexyl)-2-fluoro-5-methoxybenzamide (21 mg, 18.96 μmol, 14.64% yield, 93% purity, FA) as a yellow solid. MS(M+H)⁺=984.1

¹H NMR (400 MHz, DMSO-d₆) δ=11.09 (br s, 1H), 8.35 (s, 1H), 8.30 (s, 1H), 8.24 (d, J=13.3 Hz, 1H), 8.02 (s, 1H), 7.82 (dd, J=3.5, 7.9 Hz, 1H), 7.70-7.64 (m, 1H), 7.32 (t, J=7.0 Hz, 2H), 7.18 (d, J=6.8 Hz, 1H), 5.08 (dd, J=5.4, 12.8 Hz, 1H), 4.82 (q, J=8.0 Hz, 1H), 4.07 (t, J=13.9 Hz, 2H), 3.91 (s, 3H), 3.68 (d, J=11.3 Hz, 3H), 3.33 (s, 3H), 2.94-2.80 (m, 3H), 2.63-2.50 (m, 8H), 2.41-2.29 (m, 3H), 2.16 (d, J=7.0 Hz, 2H), 2.06-1.88 (m, 5H), 1.86-1.54 (m, 11H), 1.42-1.20 (m, 6H).

### Example 84. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((3S)-1-(3-(4-(2-(2,6-dioxopiperidin-3-yl)-1-oxoisoindolin-5-yl)piperazin-1-yl)propanoyl)pyrrolidin-3-yl)-2-fluoro-5-methoxybenzamide (Compound 84)

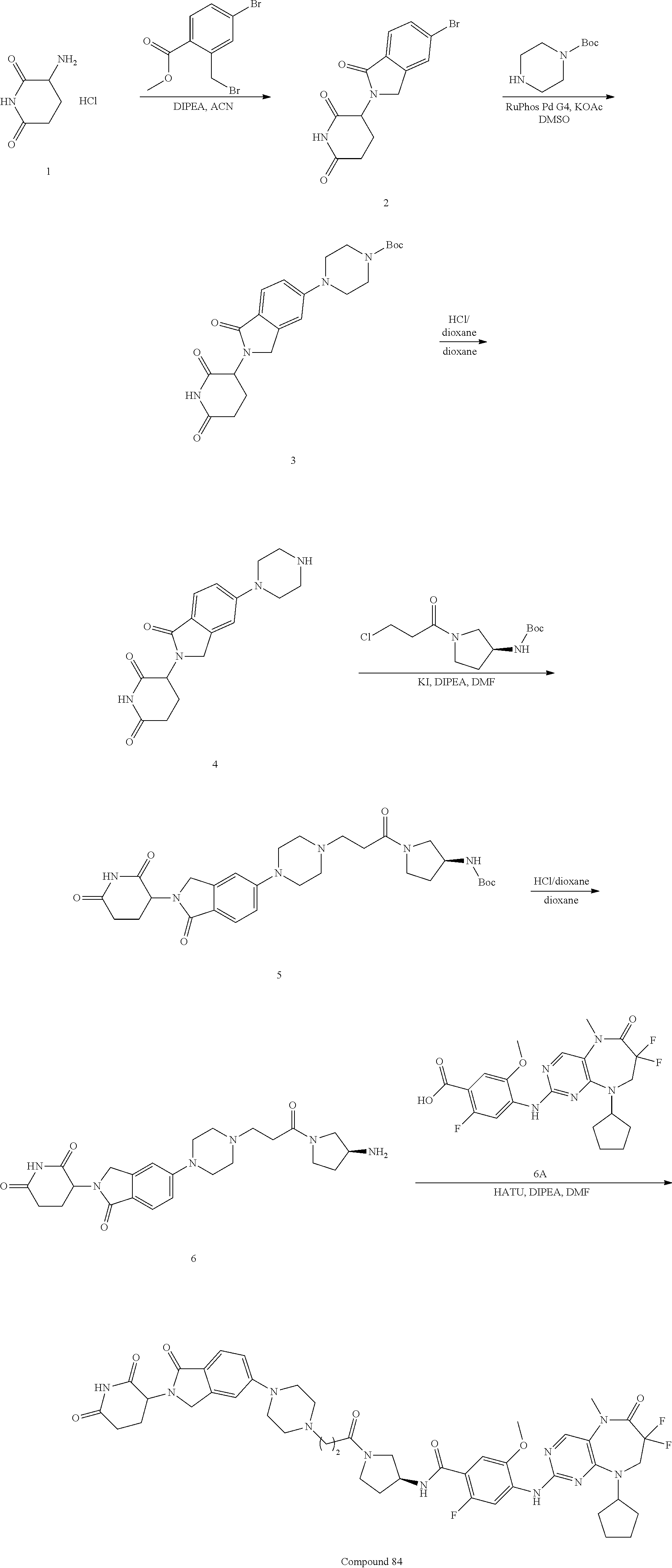

### Step 1. Synthesis of 3-(5-bromo-1-oxoisoindolin-2-yl)piperidine-2,6-dione (2)

To a solution of methyl 4-bromo-2-(bromomethyl)benzoate (6 g, 19.48 mmol) in CH₃CN (60 mL) was added DIPEA (12.61 g, 97.60 mmol, 17 mL) and 3-aminopiperidine-2,6-dione; hydrochloride (3.22 g, 19.56 mmol) at 25° C. The mixture was stirred at 90° C. for 16 hr under N₂ atmosphere. LCMS showed 34% peak with the desired mass was detected. The mixture was stirred at 25° C. for 1 hr, Then it was filtrated and the cake was washed with MTBE (20 mL×3) and dried under reduced pressure to afford 3-(5-bromo-1-oxoisoindolin-2-yl)piperidine-2,6-dione (3 g, crude) as a gray solid. MS(M+H)⁺=323.1

### Step 2. Synthesis of tert-butyl 4-(2-(2,6-dioxopiperidin-3-yl)-1-oxoisoindolin-5-yl)piperazine-1-carboxylate (3)

To a solution of 3-(5-bromo-1-oxoisoindolin-2-yl)piperidine-2,6-dione (500 mg, 1.55 mmol) in DMSO (12 mL) were added RuPhos Pd G₄ (200.00 mg, 235.18 μmol), KOAc (833.33 mg, 8.49 mmol) and tert-butyl piperazine-1-carboxylate (500.00 mg, 2.68 mmol). The mixture was stirred at 100° C. for 16 hr under N₂ atmosphere. LCMS showed 24% peak with the desired mass. The reaction mixture was filtered and to the filtrate was added H₂O (20 mL), the mixture was extracted with EtOAc (30 mL×2), the combined organic layers were washed with brine 90 mL (30 mL×3), dried over Na₂SO₄, filtered and the filtrate was concentrated under reduced pressure. The residue was purified by flash silica gel chromatography (Biotage; 20 g SepaFlash® Silica Flash Column, Eluent of 0˜10% Methanol: EtOAc ether gradient, 50 mL/min) to afford tert-butyl 4-(2-(2,6-dioxopiperidin-3-yl)-1-oxoisoindolin-5-yl)piperazine-1-carboxylate (400 mg, 933.53 μmol, 60.33% yield) as a light yellow solid. MS(M+H)⁺=429.3

### Step 3. Synthesis of 3-(1-oxo-5-(piperazin-1-yl)isoindolin-2-yl)piperidine-2,6-dione (4)

To a solution of tert-butyl 4-(2-(2,6-dioxopiperidin-3-yl)-1-oxoisoindolin-5-yl)piperazine-1-carboxylate (400 mg, 933.53 μmol) in dioxane (20 mL) were added HCl/dioxane (4 M, 20 mL) at 25° C. The mixture was stirred at 25° C. for 2 hr under N₂ atmosphere. LCMS showed main peak with the desired mass and no peak with the starting material was remained. The reaction mixture was concentrated under reduced pressure to afford 3-(1-oxo-5-(piperazin-1-yl)isoindolin-2-yl)piperidine-2,6-dione (370 mg, crude, 2HCl) as a light yellow solid. MS (M+H)⁺=329.3

### Step 4. Synthesis of tert-butyl ((3S)-1-(3-(4-(2-(2,6-dioxopiperidin-3-yl)-1-oxoisoindolin-5-yl)piperazin-1-yl)propanoyl)pyrrolidin-3-yl)carbamate (5)

To a solution of 3-(1-oxo-5-(piperazin-1-yl)isoindolin-2-yl)piperidine-2,6-dione (370 mg, 1.13 mmol) in DMF (10 mL) were added KI (200 mg, 1.20 mmol), DIPEA (890.40 mg, 6.89 mmol, 1.2 mL) and (S)-tert-butyl (1-(3-chloropropanoyl)pyrrolidin-3-yl)carbamate (1.0 g, 3.61 mmol). The mixture was stirred at 60° C. for 16 hr under N₂ atmosphere. LCMS showed the desired mass was detected and no peak with the starting material was detected. The mixture was filtered and the filtrate was purified by prep-HPLC (column: Phenomenex luna C₁₈ 150*40 mm*15 μm; mobile phase: [water (0.225% FA)-ACN]; B %: 5%-35%, 10 min, Column Temp: 30° C.) followed by lyophilization to afford tert-butyl ((3S)-1-(3-(4-(2-(2,6-dioxopiperidin-3-yl)-1-oxoisoindolin-5-yl)piperazin-1-yl)propanoyl)pyrrolidin-3-yl)carbamate (180 mg, 316.53 μmol, 28.09% yield) as a light yellow oil. MS(M+H)⁺=569.6

### Step 5. Synthesis of 3-(5-(4-(3-((S)-3-aminopyrrolidin-1-yl)-3-oxopropyl)piperazin-1-yl)-1-oxoisoindolin-2-yl)piperidine-2,6-dione (6)

To a solution of tert-butyl ((3S)-1-(3-(4-(2-(2,6-dioxopiperidin-3-yl)-1-oxoisoindolin-5-yl)piperazin-1-yl)propanoyl)pyrrolidin-3-yl)carbamate (180 mg, 316.53 μmol) in dioxane (5 mL) were added HCl/dioxane (4 M, 7.53 mL) at 25° C. The mixture was stirred at 25° C. for 2 hr under N₂ atmosphere. LCMS showed main peak with the desired mass and no peak with the starting material was remained. The reaction mixture was concentrated under reduced pressure to afford 3-(5-(4-(3-((S)-3-aminopyrrolidin-1-yl)-3-oxopropyl)piperazin-1-yl)-1-oxoisoindolin-2-yl)piperidine-2,6-dione (170 mg, crude, 2HCl) as a light yellow solid. MS(M+H)⁺=469.3

### Step 6. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((3S)-1-(3-(4-(2-(2,6-dioxopiperidin-3-yl)-1-oxoisoindolin-5-yl)piperazin-1-yl)propanoyl)pyrrolidin-3-yl)-2-fluoro-5-methoxybenzamide (Compound 84)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (120 mg, 257.83 μmol) in DMF (4 mL) were added HATU (203.08 mg, 534.09 μmol), DIPEA (222.60 mg, 1.72 mmol, 300.00 μL) and 3-(5-(4-(3-((S)-3-aminopyrrolidin-1-yl)-3-oxopropyl)piperazin-1-yl)-1-oxoisoindolin-2-yl)piperidine-2,6-dione (160 mg, 295.49 μmol, 2HCl) at 25° C. The mixture was stirred at 25° C. for 16 hr under N₂ atmosphere. LCMS showed the 54% desired mass was detected. To the reaction mixture was added H₂O (8 mL), the mixture was extracted with EtOAc (20 mL×2), the combined organic layers were washed with brine 60 mL (20 mL×3), dried over anhydrous Na₂SO₄, filtered and the filtrate was concentrated under reduced pressure. The residue was purified by prep-HPLC (column: 3_Phenomenex Luna C₁₈ 75*30 mm*3 μm; mobile phase: [water (0.1% TFA)-ACN]; B %: 28%-48%, 7 min, Column Temp: 30° C.) and re-purified by prep-HPLC (column: Waters Xbridge C₁₈ 150*50 mm*10 μm; mobile phase: [water (10 mM NH₄HCO₃)-ACN]; B %: 26%-56%, 11 min, Column Temp: 30° C.) followed by lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-((3S)-1-(3-(4-(2-(2,6-dioxopiperidin-3-yl)-1-oxoisoindolin-5-yl)piperazin-1-yl)propanoyl)pyrrolidin-3-yl)-2-fluoro-5-methoxybenzamide (78.0 mg, 80.05 μmol, 31.05% yield, 94% purity) as a white solid. MS(M+H)⁺=916.6

¹H NMR (400 MHz, DMSO-d₆) δ=10.94 (s, 1H), 8.35-8.20 (m, 3H), 8.04 (s, 1H), 7.57-7.44 (m, 1H), 7.20 (d, J=6.5 Hz, 1H), 7.11-7.00 (m, 2H), 5.08-4.99 (m, 1H), 4.86-4.76 (m, 1H), 4.52-4.38 (m, 1H), 4.35-4.27 (m, 1H), 4.24-4.15 (m, 1H), 4.12-4.02 (m, 2H), 3.91 (d, J=3.0 Hz, 3H), 3.81-3.55 (m, 2H), 3.53-3.39 (m, 2H), 3.33-3.32 (m, 3H), 3.30-3.26 (m, 4H), 2.95-2.85 (m, 1H), 2.64-2.51 (m, 9H), 2.38-2.07 (m, 2H), 2.02-1.86 (m, 4H), 1.76-1.55 (m, 6H).

### Example 85. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((3S)-1-(4-(4-(2-(2,6-dioxopiperidin-3-yl)-1-oxoisoindolin-4-yl)piperazin-1-yl)-4-oxobutanoyl)pyrrolidin-3-yl)-3-methoxybenzamide (Compound 85)

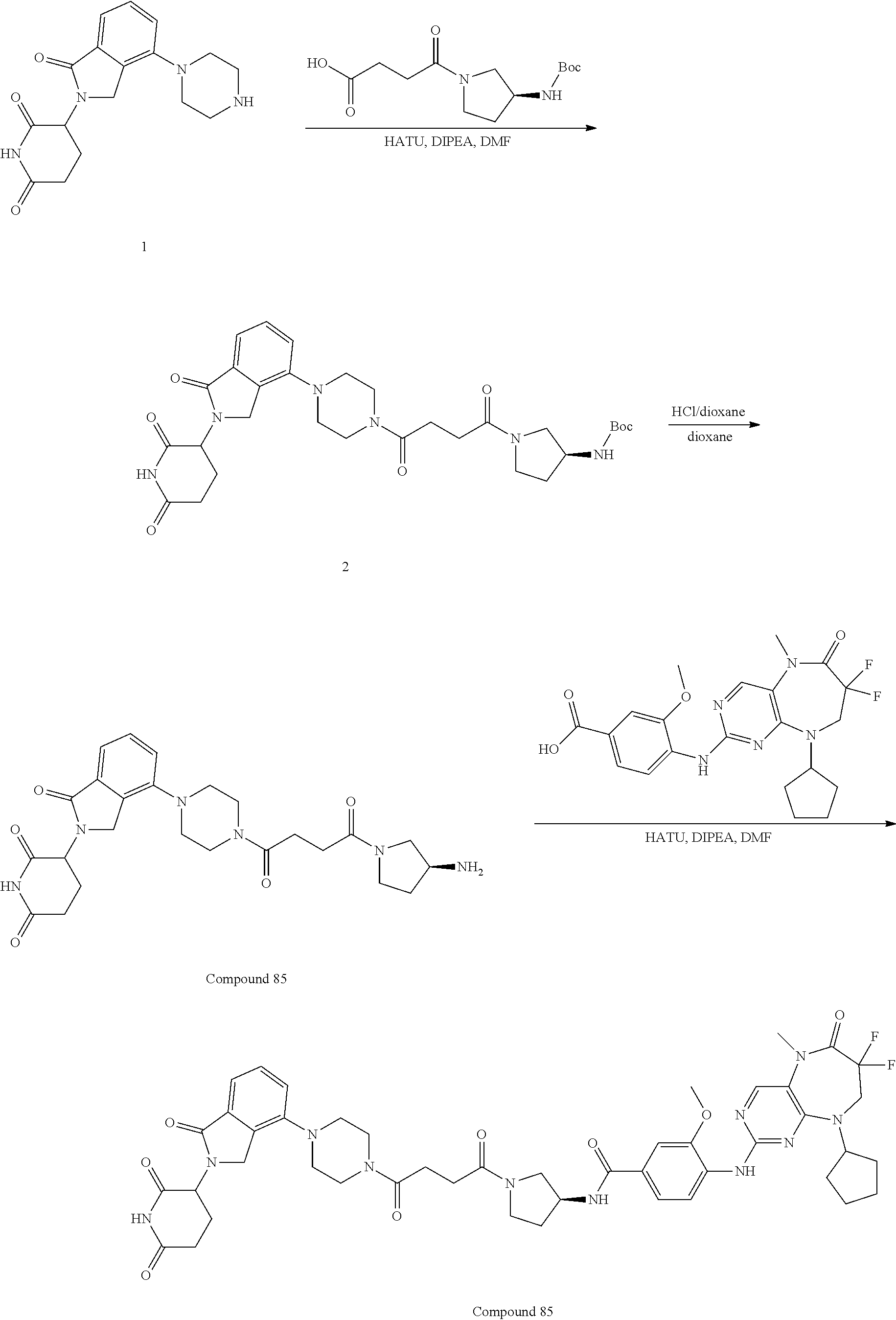 [SMILES: O=C1CCC(N2Cc3c(cccc3N3CCNCC3)C2=O)C(=O)N1 ; CC(C)(C)OC(=O)N[C@H]1CCN(C(=O)CCC(=O)O)C1 ; CC(C)(C)OC(=O)N[C@H]1CCN(C(=O)CCC(=O)N2CCN(c3cccc4c3CN(C3CCC(=O)NC3=O)C4=O)CC2)C1 ; N[C@H]1CCN(C(=O)CCC(=O)N2CCN(c3cccc4c3CN(C3CCC(=O)NC3=O)C4=O)CC2)C1 ; COc1cc(C(=O)O)ccc1Nc1ncc2c(n1)N(C1CCCC1)CC(F)(F)C(=O)N2C ; COc1cc(C(=O)N[C@H]2CCN(C(=O)CCC(=O)N3CCN(c4cccc5c4CN(C4CCC(=O)NC4=O)C5=O)CC3)C2)ccc1Nc1ncc2c(n1)N(C1CCCC1)CC(F)(F)C(=O)N2C]

The compound 85 was synthesized by the method described in the scheme similar to the method described in Example 81.

MS(M+H)⁺=926.4, ¹H NMR (400 MHz, DMSO-d₆) δ=8.49-8.38 (m, 1H), 8.31-8.25 (m, 2H), 7.99 (d, J=2.3 Hz, 1H), 7.53-7.48 (m, 2H), 7.48-7.42 (m, 1H), 7.35 (d, J=7.4 Hz, 1H), 7.18 (d, J=7.6 Hz, 1H), 5.17-5.09 (m, 1H), 4.82-4.71 (m, 1H), 4.57-4.39 (m, 2H), 4.36-4.27 (m, 1H), 4.05 (br t, J=13.9 Hz, 2H), 3.94 (s, 3H), 3.65-3.58 (m, 4H), 3.45-3.36 (m, 1H), 3.33-3.30 (m, 3H), 3.30-3.23 (m, 1H), 3.13-2.87 (m, 5H), 2.64-2.56 (m, 3H), 2.49-2.48 (m, 4H), 2.27-2.17 (m, 1H), 2.16-2.05 (m, 1H), 2.03-1.90 (m, 4H), 1.75-1.67 (m, 2H), 1.65-1.52 (m, 4H).

### Example 86. Synthesis of 4-(4-(2-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperazin-1-yl)ethyl)piperazin-1-yl)-2-(2,6-dioxopiperidin-3-yl)isoindoline-1,3-dione (Compound 86)

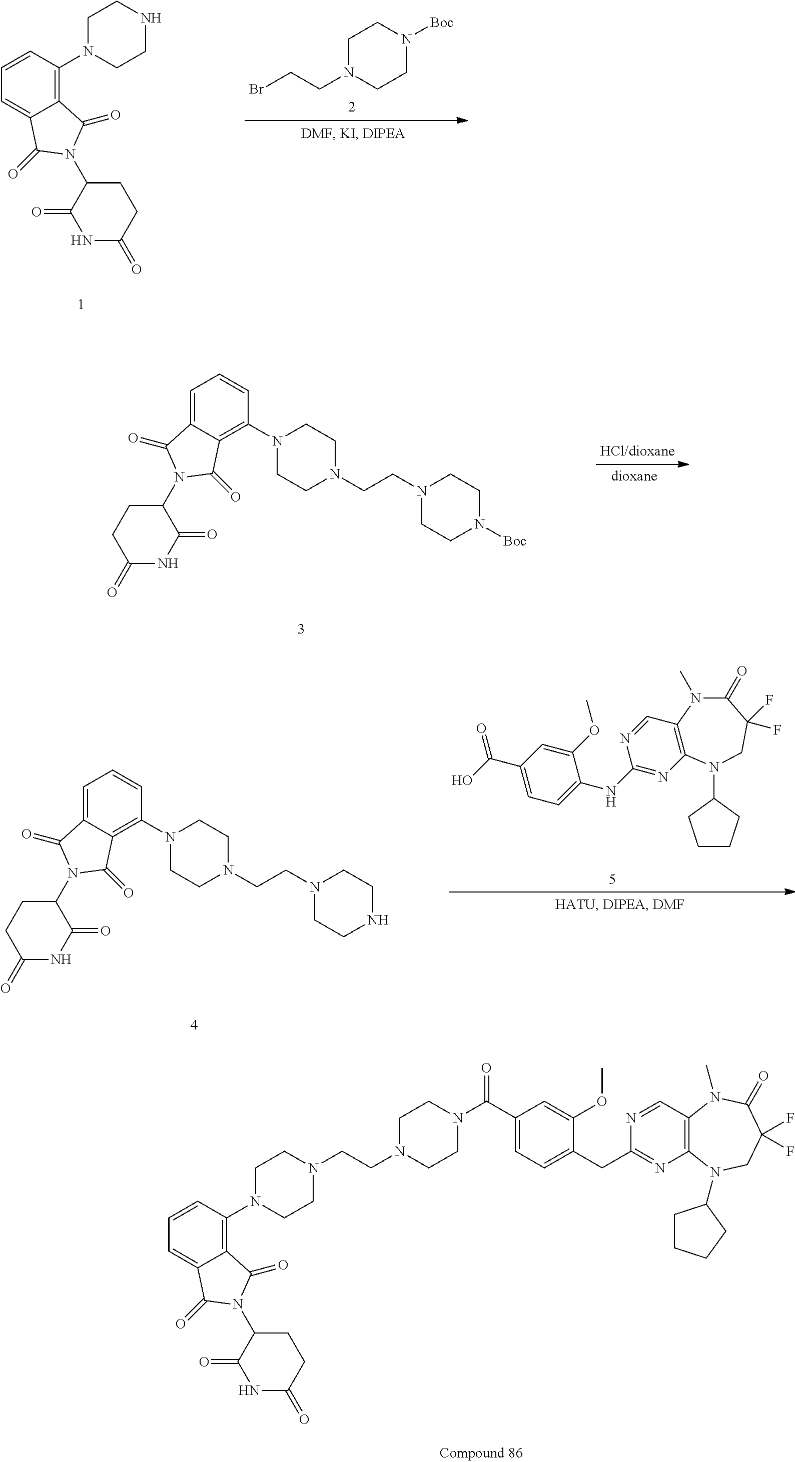 [SMILES: O=C1CCC(N2C(=O)c3cccc(N4CCNCC4)c3C2=O)C(=O)N1 ; CC(C)(C)OC(=O)N1CCN(CCBr)CC1 ; CC(C)(C)OC(=O)N1CCN(CCN2CCN(c3cccc4c3C(=O)N(C3CCC(=O)NC3=O)C4=O)CC2)CC1 ; O=C1CCC(N2C(=O)c3cccc(N4CCN(CCN5CCNCC5)CC4)c3C2=O)C(=O)N1 ; COc1cc(C(=O)O)ccc1Nc1ncc2c(n1)N(C1CCCC1)CC(F)(F)C(=O)N2C ; COc1cc(C(=O)N2CCN(CCN3CCN(c4cccc5c4C(=O)N(C4CCC(=O)NC4=O)C5=O)CC3)CC2)ccc1Cc1ncc2c(n1)N(C1CCCC1)CC(F)(F)C(=O)N2C]

The compound 86 was synthesized by the method described in the scheme similar to the method described in Example 81.

MS (M+H)⁺=884.4, ¹H NMR (400 MHz, DMSO-d₆) δ=11.08 (s, 1H), 8.23 (s, 1H), 8.15 (d, J=8.4 Hz, 1H), 7.99 (s, 1H), 7.69 (t, J=0.8 Hz, 1H), 7.34 (t, J=7.2 Hz, 2H), 7.04 (d, J=1.6 Hz, 1H), 6.96 (dd, J=1.2, 8.0 Hz, 1H), 5.10-5.06 (m, 1H), 4.74-4.66 (m, 1H), 4.02 (t, J=14 Hz, 2H), 3.88 (s, 3H), 3.56-3.43 (m, 4H), 3.32 (s, 3H), 3.29-3.27 (m, 4H), 2.88-2.82 (m, 2H), 2.59-2.51 (m, 6H), 2.47-2.43 (m, 6H), 2.03-1.89 (m, 4H), 1.70-1.64 (m, 2H), 1.58-1.54 (m, 4H).

### Example 87. Synthesis of 4-(4-(3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperazin-1-yl)propyl)piperazin-1-yl)-2-(2,6-dioxopiperidin-3-yl)isoindoline-1,3-dione (Compound 87)

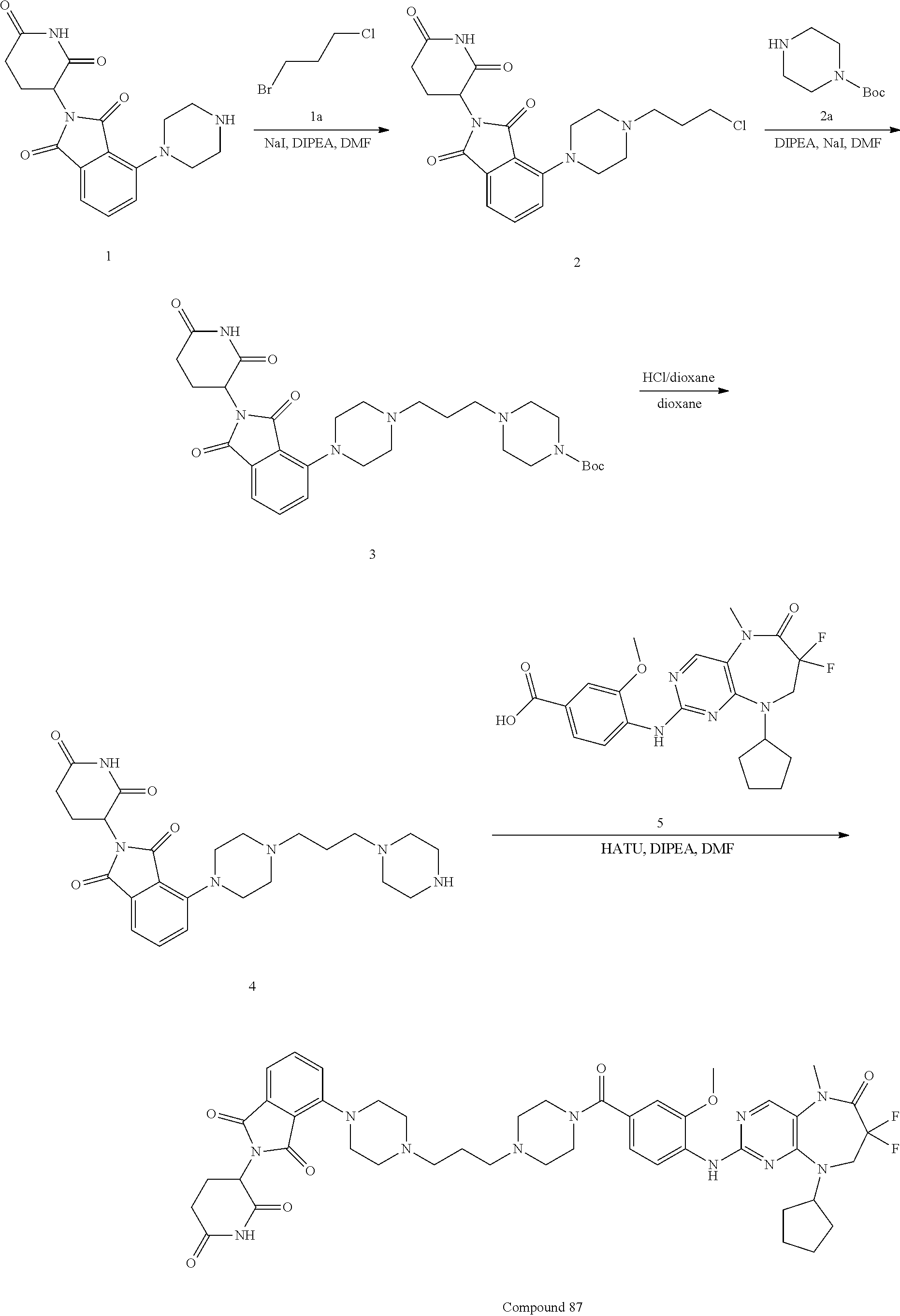

The compound 87 was synthesized by the method described in the scheme similar to the method described in Example 60.

MS(M+H)⁺=898.6, ¹H NMR (400 MHz, DMSO-d₆) δ=11.18-10.98 (m, 1H), 8.23 (s, 1H), 8.15 (d, J=8.1 Hz, 1H), 7.98 (s, 1H), 7.70 (dd, J=7.4, 8.2 Hz, 1H), 7.38-7.30 (m, 2H), 7.04 (d, J=1.6 Hz, 1H), 6.96 (dd, J=1.5, 8.3 Hz, 1H), 5.08 (dd, J=5.4, 12.7 Hz, 1H), 4.77-4.65 (m, 1H), 4.02 (t, J=14.2 Hz, 2H), 3.88 (s, 3H), 3.58-3.47 (m, 4H), 3.32 (s, 3H), 3.30-3.26 (m, 4H), 2.94-2.80 (m, 1H), 2.63-2.53 (m, 6H), 2.43-2.30 (m, 8H), 2.08-1.97 (m, 1H), 1.96-1.83 (m, 2H), 1.73-1.51 (m, 8H).

### Example 88. Synthesis of 4-(4-(4-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperazin-1-yl)butyl)piperazin-1-yl)-2-(2,6-dioxopiperidin-3-yl)isoindoline-1,3-dione (Compound 88)

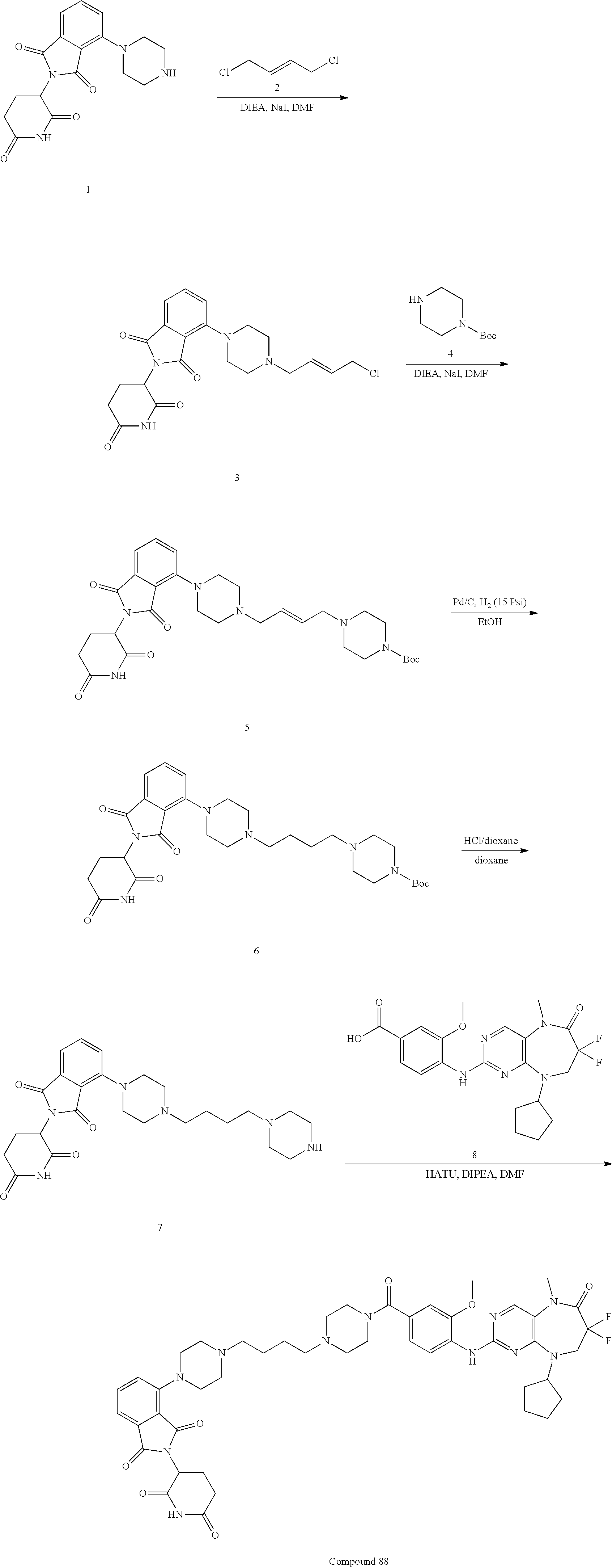

### Step 1. Synthesis of (E)-4-(4-(4-chlorobut-2-en-1-yl)piperazin-1-yl)-2-(2,6-dioxopiperidin-3-yl)isoindoline-1,3-dione (3)

To a solution of 2-(2,6-dioxopiperidin-3-yl)-4-(piperazin-1-yl)isoindoline-1,3-dione (1 g, 2.64 mmol, HCl) and (E)-1,4-dichlorobut-2-ene (1.66 g, 13.28 mmol, 1.41 mL) in DMF (10 mL) were added DIEA (1.04 g, 8.04 mmol, 1.40 mL) and NaI (7.91 mg, 52.80 μmol) and the mixture was stirred at 20° C. for 14 h. TLC (Petroleum ether:EtOAc=10:1) showed the desired spot was detected. The mixture was diluted with H₂O (10 mL) and extracted with EtOAc (10 mL×3), the combined organic layer was washed with brine (10 mL×3), dried over Na₂SO₄ and filtered. The filtrate was concentrated under reduced pressure. The residue was purified by flash silica gel chromatography (12 g SepaFlash® Silica Flash Column, Eluent of 10˜100% EtOAc/Petroleum ether to 10-100% EtOH/EtOAc gradient @50 mL/min) to afford (E)-4-(4-(4-chlorobut-2-en-1-yl)piperazin-1-yl)-2-(2,6-dioxopiperidin-3-yl)isoindoline-1,3-dione (460 mg, 1.07 mmol, 40.44% yield) as a yellow solid. MS(M+H)⁺=431.1

### Step 2. Synthesis of (E)-tert-butyl 4-(4-(4-(2-(2,6-dioxopiperidin-3-yl)-1,3-dioxoisoindolin-4-yl)piperazin-1-yl)but-2-en-1-yl)piperazine-1-carboxylate (5)

To a solution of (E)-4-(4-(4-chlorobut-2-en-1-yl)piperazin-1-yl)-2-(2,6-dioxopiperidin-3-yl)isoindoline-1,3-dione (460 mg, 1.07 mmol) and tert-butyl piperazine-1-carboxylate (285.31 mg, 1.28 mmol) in DMF (10 mL) were added NaI (8.00 mg, 53.38 μmol) and DIPEA (413.93 mg, 3.20 mmol, 557.86 μL) and the mixture was stirred at 20° C. for 14 h. LCMS showed 91% of the desired mass was detected. The mixture was diluted with H₂O (10 mL) and extracted with EtOAc (10 mL×3), the combined organic layer was washed with brine (10 mL×3), dried over Na₂SO₄ and filtered. The filtrate was concentrated under reduced pressure. The residue was diluted with MTBE (10 mL) and stirred at 20° C. for 1 h. The mixture was filtered and the filter cake was washed with MTBE (10 mL). The filter cake was collected and dried under reduced pressure to afford (E)-tert-butyl 4-(4-(4-(2-(2,6-dioxopiperidin-3-yl)-1,3-dioxoisoindolin-4-yl)piperazin-1-yl)but-2-en-1-yl)piperazine-1-carboxylate (0.4 g, 681.97 μmol, 63.88% yield, 99% purity) as a yellow solid. MS(M+H)⁺=581.3

### Step 3. Synthesis of tert-butyl 4-(4-(4-(2-(2,6-dioxopiperidin-3-yl)-1,3-dioxoisoindolin-4-yl)piperazin-1-yl)butyl)piperazine-1-carboxylate (6)

To a solution of (E)-tert-butyl 4-(4-(4-(2-(2,6-dioxopiperidin-3-yl)-1,3-dioxoisoindolin-4-yl)piperazin-1-yl)but-2-en-1-yl)piperazine-1-carboxylate (200 mg, 344.43 μmol) in EtOH (6 mL) was added Pd/C (30 mg, 10% purity) and the mixture was stirred at 20° C. under H₂ (15 Psi) for 14 h. LCMS showed 88% of the desired mass was detected. The mixture was diluted with THF (10 mL) and then filtered and the filter cake was washed with EtOH (20 mL) and THF (10 mL). The filtrate was concentrated under reduced pressure to afford tert-butyl 4-(4-(4-(2-(2,6-dioxopiperidin-3-yl)-1,3-dioxoisoindolin-4-yl)piperazin-1-yl)butyl)piperazine-1-carboxylate (150 mg, 239.41 μmol, 69.51% yield, 93% purity) as a yellow solid. MS(M+H)⁺=583.4

### Step 4. Synthesis of 2-(2,6-dioxopiperidin-3-yl)-4-(4-(4-(piperazin-1-yl)butyl)piperazin-1-yl)isoindoline-1,3-dione (7)

To a solution of tert-butyl 4-[4-[4-[2-(2,6-dioxo-3-piperidyl)-1,3-dioxo-isoindolin-4-yl]piperazin-1-yl]butyl]piperazine-1-carboxylate (150 mg, 257.43 μmol) in dioxane (2 mL) was added HCl/dioxane (4 M, 4 mL) and the mixture was stirred at 20° C. for 2 h. LCMS showed 76% of the desired mass was detected after work up. The mixture was concentrated under reduced pressure to afford 2-(2,6-dioxopiperidin-3-yl)-4-(4-(4-(piperazin-1-yl)butyl)piperazin-1-yl)isoindoline-1,3-dione (120 mg, crude, HCl) as a yellow solid. MS(M+H)⁺=483.1

### Step 5. Synthesis of 4-(4-(4-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperazin-1-yl)butyl)piperazin-1-yl)-2-(2,6-dioxopiperidin-3-yl)isoindoline-1,3-dione (Compound 88)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (80 mg, 178.80 μmol) and HATU (101.98 mg, 268.20 μmol) in DMF (1 mL) was added DIPEA (46.22 mg, 357.59 μmol, 62.29 μL) and the mixture was stirred at 20° C. for 15 min. Then a solution of 2-(2,6-dioxopiperidin-3-yl)-4-(4-(4-(piperazin-1-yl)butyl)piperazin-1-yl)isoindoline-1,3-dione (120 mg, 231.20 μmol, HCl) and DIPEA (69.32 mg, 536.39 μmol, 93.43 μL) in DMF (2 mL) was added and the mixture was stirred at 20° C. for 45 min. LCMS showed 65% of the desired mass was detected. The mixture was diluted with H₂O (10 mL) and extracted with EtOAc (10 mL×3), the combined organic layer was washed with brine (10 mL×3), dried over Na₂SO₄ and filtered. The filtrate was concentrated under reduced pressure. The residue was purified by prep-TLC (Dichloromethane:Methanol=10:1) to afford 4-(4-(4-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperazin-1-yl)butyl)piperazin-1-yl)-2-(2,6-dioxopiperidin-3-yl)isoindoline-1,3-dione (47.4 mg, 48.34 μmol, 27.03% yield, 93% purity) as a yellow solid. MS(M+H)⁺=912.2

¹H NMR (400 MHz, DMSO-d₆) δ=11.08 (s, 1H), 8.23 (s, 1H), 8.15 (d, J=8.3 Hz, 1H), 7.98 (s, 1H), 7.70 (t, J=7.8 Hz, 1H), 7.34 (t, J=7.9 Hz, 2H), 7.04 (s, 1H), 6.96 (d, J=8.1 Hz, 1H), 5.13-5.05 (m, 1H), 4.76-4.66 (m, 1H), 4.02 (t, J=14.2 Hz, 2H), 3.88 (s, 3H), 3.58-3.42 (m, 4H), 3.31-3.25 (m, 7H), 2.93-2.81 (m, 1H), 2.68-2.53 (m, 6H), 2.41-2.29 (m, 8H), 2.06-1.97 (m, 1H), 1.95-1.85 (m, 2H), 1.71-1.63 (m, 2H), 1.62-1.44 (m, 8H)

### Example 89. Synthesis of 5-(4-(2-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperazin-1-yl)ethyl)piperazin-1-yl)-2-(2,6-dioxopiperidin-3-yl)isoindoline-1,3-dione (Compound 89)

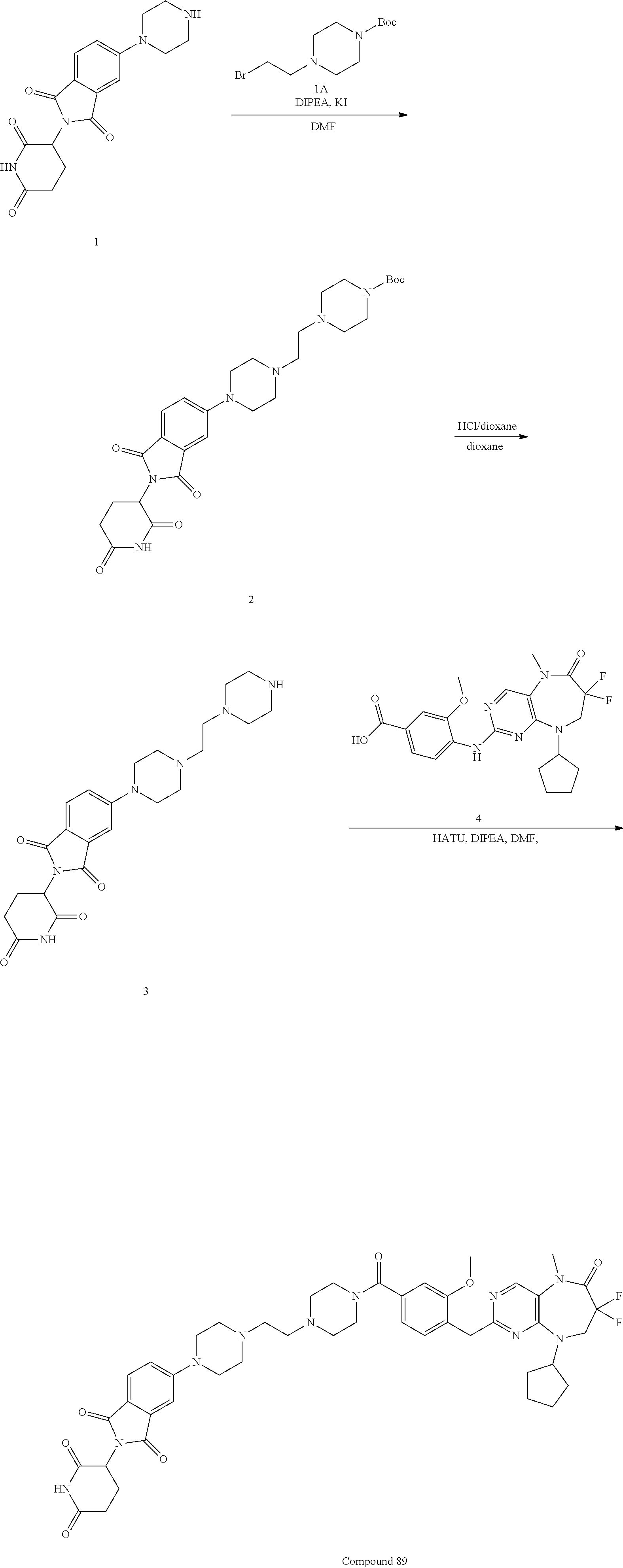

### Step 1. Synthesis of tert-butyl 4-(2-(4-(2-(2,6-dioxopiperidin-3-yl)-1,3-dioxoisoindolin-5-yl)piperazin-1-yl)ethyl)piperazine-1-carboxylate (2)

A mixture 2-(2,6-dioxopiperidin-3-yl)-5-(piperazin-1-yl)isoindoline-1,3-dione (0.4 g, 1.17 mmol), tert-butyl 4-(2-bromoethyl)piperazine-1-carboxylate (342.57 mg, 1.17 mmol), KI (19.40 mg, 116.84 μmol) and DIPEA (453.02 mg, 3.51 mmol, 610.54 μL) in DMF (5 mL) was stirred at 70° C. for 12 hr. LCMS showed the 2-(2,6-dioxopiperidin-3-yl)-5-(piperazin-1-yl)isoindoline-1,3-dione was consumed completely, and a main peak with desired mass. The mixture was diluted with EtOAc (20 mL) concentrated under reduced pressure. The residue was purified by flash silica gel chromatography (10 g SepaFlash® Silica Flash Column, Eluent of 0˜100% EtOAc/Petroleum ether gradient @65 mL/min; Eluent of 0˜50% Methanol/EtOAc @65 mL/min) to afford tert-butyl 4-(2-(4-(2-(2,6-dioxopiperidin-3-yl)-1,3-dioxoisoindolin-5-yl)piperazin-1-yl)ethyl)piperazine-1-carboxylate (0.6 g, 1.05 mmol, 89.81% yield, 97% purity) as yellow solid, which was used for the next step directly. MS(M+H)⁺=555.2

¹H NMR (400 MHz, CDCl3) δ=8.18 (br s, 1H), 7.71 (d, J=8.5 Hz, 1H), 7.29 (d, J=2.3 Hz, 1H), 7.07 (dd, J=2.3, 8.6 Hz, 1H), 5.00-4.90 (m, 1H), 3.76-3.52 (m, 4H), 3.52-3.42 (m, 4H), 3.02-2.65 (m, 15H), 2.20-2.10 (m, 1H), 1.47 (s, 9H).

### Step 2. Synthesis of 2-(2,6-dioxopiperidin-3-yl)-5-(4-(2-(piperazin-1-yl)ethyl)piperazin-1-yl)isoindoline-1,3-dione (3)

To a solution of tert-butyl 4-(2-(4-(2-(2,6-dioxopiperidin-3-yl)-1,3-dioxoisoindolin-5-yl)piperazin-1-yl)ethyl)piperazine-1-carboxylate (0.6 g, 1.08 mmol) in dioxane (2 mL) was added HCl/dioxane (4 M, 20 mL) at 25° C. The resulting mixture was stirred at 25° C. for 0.5 hr. TLC (Dichloromethane:Methanol=10:1; Rf=0.5) showed the starting material was consumed completely and found new spot. The mixture solution was concentrated under reduced pressure to give 2-(2,6-dioxopiperidin-3-yl)-5-(4-(2-(piperazin-1-yl)ethyl)piperazin-1-yl)isoindoline-1,3-dione (0.4 g, HCl) as yellow solid, which was used for the next step directly. MS(M+H)⁺=455.1

### Step 3. Synthesis of 5-(4-(2-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperazin-1-yl)ethyl)piperazin-1-yl)-2-(2,6-dioxopiperidin-3-yl)isoindoline-1,3-dione (Compound 89)

To a solution of 2-(2,6-dioxopiperidin-3-yl)-5-(4-(2-(piperazin-1-yl)ethyl)piperazin-1-yl)isoindoline-1,3-dione (76.81 mg, 156.45 μmol, HCl) in DMF (1 mL) was added HATU (89.23 mg, 234.67 μmol) and DIPEA (60.66 mg, 469.34 μmol, 81.75 μL). The mixture was stirred at 25° C. for 10 min. To mixture was added 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (70 mg, 156.45 μmol). The mixture was stirred at 25° C. for 12 h. LCMS showed the 2-(2,6-dioxopiperidin-3-yl)-5-(4-(2-(piperazin-1-yl)ethyl)piperazin-1-yl)isoindoline-1,3-dione was consumed completely and peak (67%) with desired mass. The reaction mixture was purified by prep-HPLC (column: Phenomenex Synergi Polar-RP 100*25 mm*4 μm; mobile phase: [water(TFA)-ACN]; B %: 28%-48%, 7 min) and re-purified by prep-HPLC (column: Waters Xbridge 150*25 mm*5 μm; mobile phase: [water(NH₄HCO₃)-ACN]; B %: 38%-68%, min) and lyophilized to afford 5-(4-(2-(4-(4-((9-cyclopentyl-7,7-difluoro-5-methyl-6-oxo-6,7,8,9-tetrahydro-5H-pyrimido [4,5-b][1,4]diazepin-2-yl)amino)-3-methoxybenzoyl)piperazin-1-yl)ethyl)piperazin-1-yl)-2-(2,6-dioxopiperidin-3-yl)isoindoline-1,3-dione (34.9 mg, 37.51 μmol, 23.97% yield, 95% purity) as yellow solid. MS(M+H)⁺=884.5

¹H NMR (400 MHz, DMSO-d₆) δ=11.08 (br s, 1H), 8.23 (s, 1H), 8.15 (d, J=8.2 Hz, 1H), 8.00 (s, 1H), 7.67 (d, J=8.6 Hz, 1H), 7.33 (d, J=1.8 Hz, 1H), 7.25 (dd, J=2.0, 8.7 Hz, 1H), 7.04 (d, J=1.6 Hz, 1H), 6.96 (dd, J=1.7, 8.3 Hz, 1H), 5.07 (dd, J=5.4, 12.8 Hz, 1H), 4.80-4.63 (m, 1H), 4.02 (t, J=14.1 Hz, 2H), 3.88 (s, 3H), 3.68-3.45 (m, 4H), 3.44-2.41 (m, 4H), 3.32 (s, 3H), 2.96-2.81 (m, 1H), 2.64-2.51 (m, 11H), 2.46-2.42 (m, 3H), 2.08-1.97 (m, 1H), 1.96-1.83 (m, 2H), 1.75-1.63 (m, 2H), 1.62-1.45 (m, 4H).

### Example 90. Synthesis of 5-(4-(3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperazin-1-yl)propyl)piperazin-1-yl)-2-(2,6-dioxopiperidin-3-yl)isoindoline-1,3-dione (Compound 90)

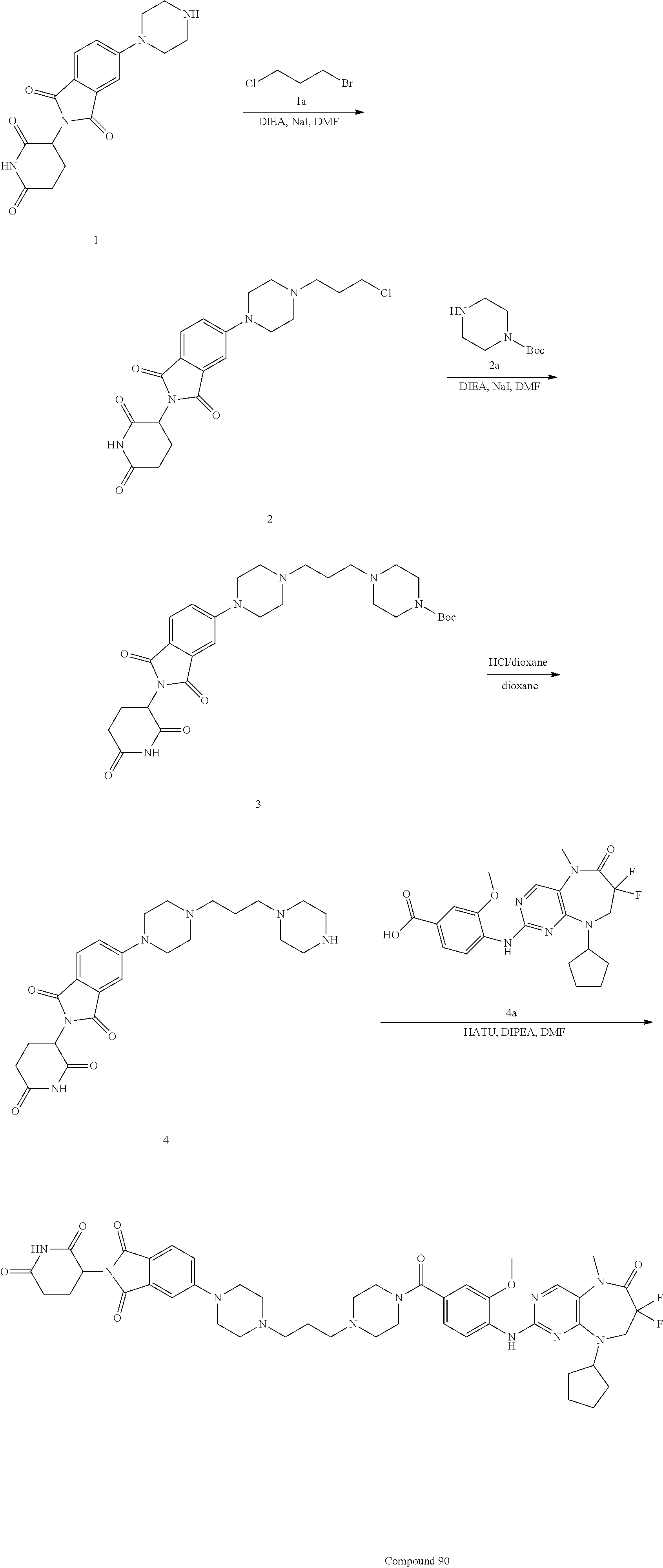

The compound 90 was synthesized by the method described in the scheme similar to the method described in Example 60.

MS(M+H)⁺=898.6, ¹H NMR (400 MHz, DMSO-d₆) δ 11.33-10.77 (m, 1H), 8.23 (s, 1H), 8.15 (d, J=8.1 Hz, 1H), 7.98 (s, 1H), 7.67 (d, J=8.5 Hz, 1H), 7.33 (d, J=2.0 Hz, 1H), 7.25 (dd, J=2.2, 8.7 Hz, 1H), 7.04 (d, J=1.8 Hz, 1H), 6.96 (dd, J=1.7, 8.2 Hz, 1H), 5.12-5.00 (m, 1H), 4.80-4.63 (m, 1H), 4.02 (t, J=14.1 Hz, 2H), 3.88 (s, 3H), 3.71-3.39 (m, 8H), 3.30 (s, 3H), 2.95-2.80 (m, 1H), 2.62-2.51 (m, 6H), 2.43-2.29 (m, 8H), 2.08-1.97 (m, 1H), 1.96-1.82 (m, 2H), 1.71-1.50 (m, 8H).

### Example 91. Synthesis of 5-(4-(4-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperazin-1-yl)butyl)piperazin-1-yl)-2-(2,6-dioxopiperidin-3-yl)isoindoline-1,3-dione (Compound 91)

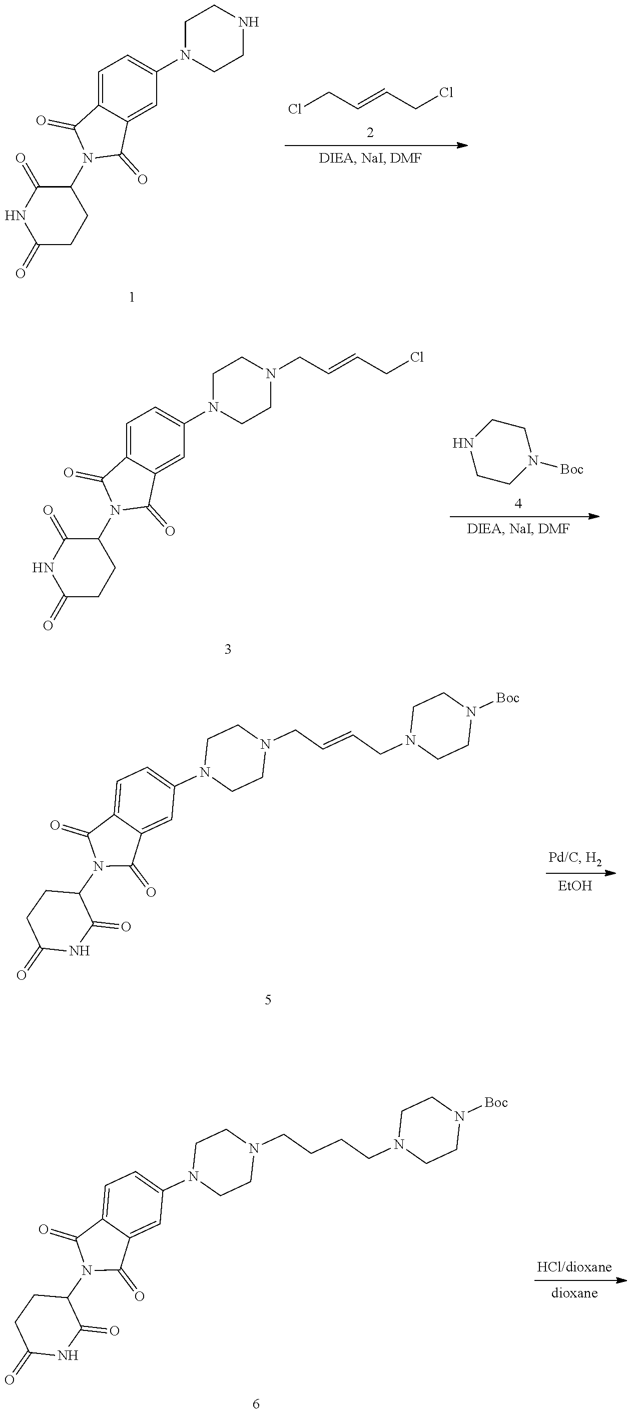

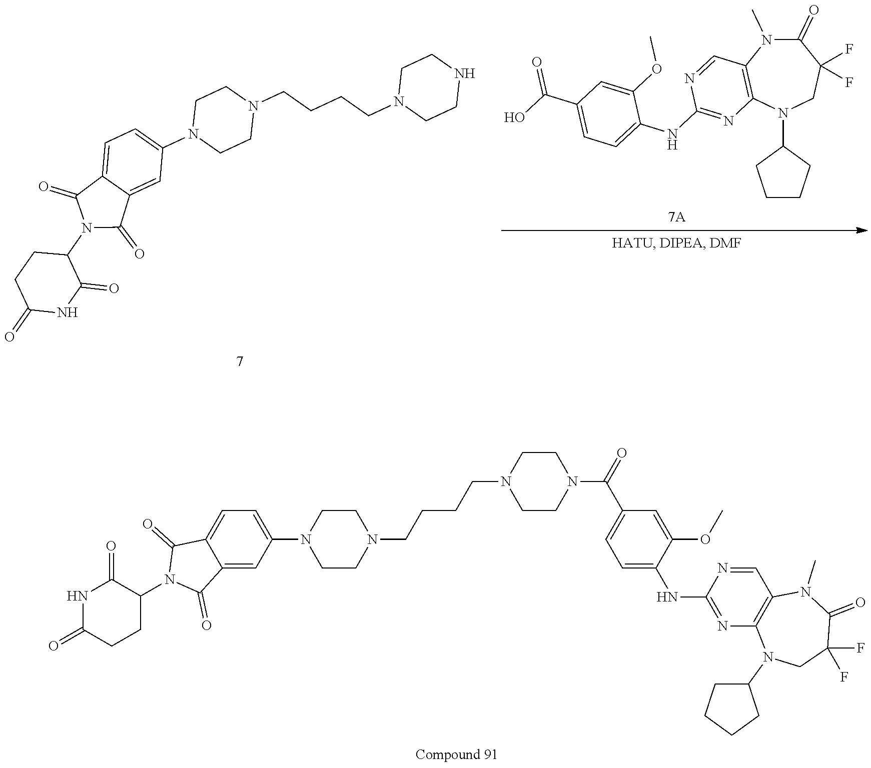 [SMILES: O=C1CCC(N2C(=O)c3ccc(N4CCN(CCCCN5CCNCC5)CC4)cc3C2=O)C(=O)N1 ; COc1cc(C(=O)O)ccc1Nc1ncc2c(n1)N(C1CCCC1)CC(F)(F)C(=O)N2C ; "HATU, DIPEA, DMF" ; COc1cc(C(=O)N2CCN(CCCCN3CCN(c4ccc5c(c4)C(=O)N(C4CCC(=O)NC4=O)C5=O)CC3)CC2)ccc1Nc1ncc2c(n1)N(C1CCCC1)CC(F)(F)C(=O)N2C]

The compound 91 was synthesized by the method described in the scheme similar to the method described in Example 88.

MS(M+H)⁺=912.2, ¹H NMR (400 MHz, DMSO-d₆) δ=11.01 (s, 1H), 8.23 (s, 1H), 8.15 (d, J=8.2 Hz, 1H), 7.99 (s, 1H), 7.67 (d, J=8.4 Hz, 1H), 7.33 (d, J=1.7 Hz, 1H), 7.25 (dd, J=1.8, 8.7 Hz, 1H), 7.03 (d, J=1.5 Hz, 1H), 7.00-6.91 (m, 1H), 5.07 (dd, J=5.3, 12.9 Hz, 1H), 4.81-4.63 (m, 1H), 4.02 (br t, J=14.1 Hz, 2H), 3.88 (s, 3H), 3.42 (br d, J=4.4 Hz, 8H), 3.32 (s, 3H), 2.95-2.80 (m, 1H), 2.63-2.56 (m, 2H), 2.55-2.41 (m, 4H), 2.43-2.25 (m, 8H), 2.07-1.96 (m, 1H), 1.96-1.82 (m, 2H), 1.73-1.63 (br s, 2H), 1.63-1.51 (m, 4H), 1.48-1.35 (m, 4H).

### Example 92. Synthesis of (E)-5-(4-(4-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperazin-1-yl)but-2-en-1-yl)piperazin-1-yl)-2-(2,6-dioxopiperidin-3-yl)isoindoline-1,3-dione (Compound 92)

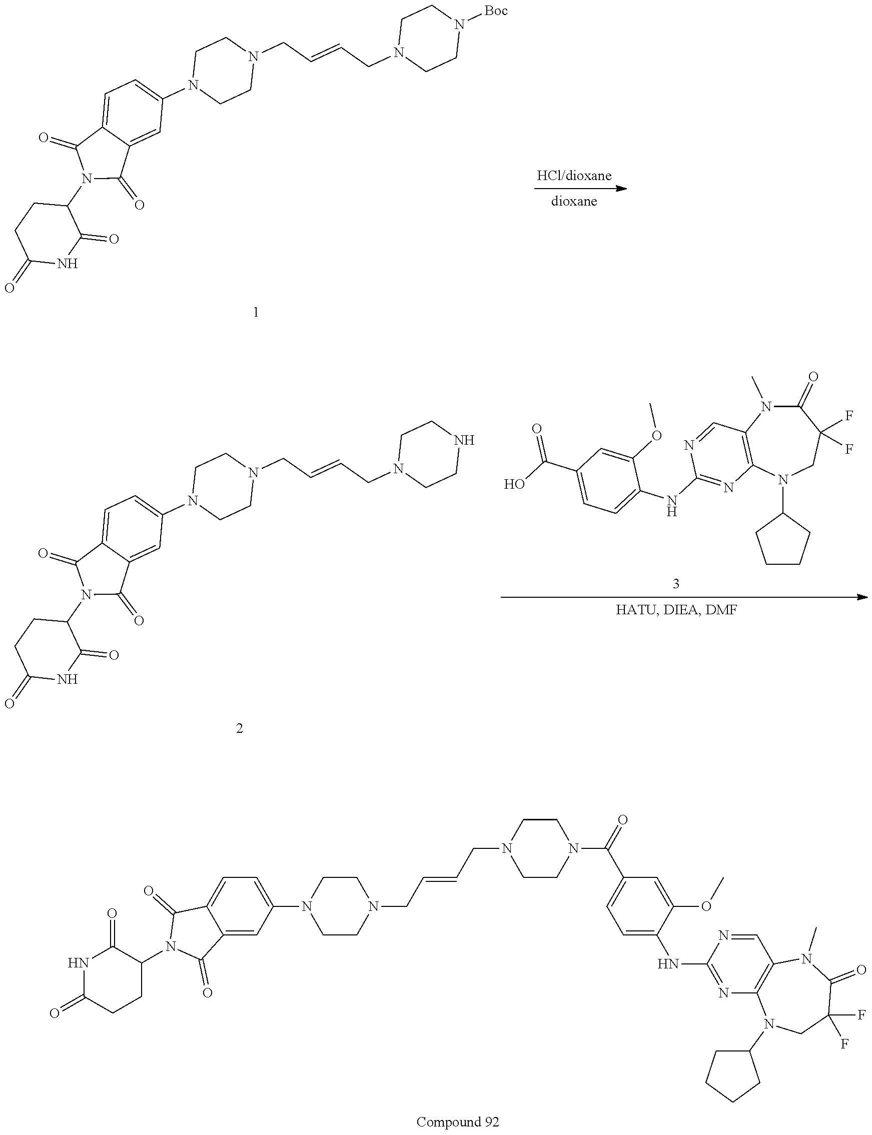 [SMILES: CC(C)(C)OC(=O)N1CCN(C/C=C/CN2CCN(c3ccc4c(c3)C(=O)N(C3CCC(=O)NC3=O)C4=O)CC2)CC1 ; "dioxane" ; O=C1CCC(N2C(=O)c3ccc(N4CCN(C/C=C/CN5CCNCC5)CC4)cc3C2=O)C(=O)N1 ; COc1cc(C(=O)O)ccc1Nc1ncc2c(n1)N(C1CCCC1)CC(F)(F)C(=O)N2C ; "HATU, DIEA, DMF" ; COc1cc(C(=O)N2CCN(C/C=C/CN3CCN(c4ccc5c(c4)C(=O)N(C4CCC(=O)NC4=O)C5=O)CC3)CC2)ccc1Nc1ncc2c(n1)N(C1CCCC1)CC(F)(F)C(=O)N2C]

The compound 92 was synthesized by the method described in the scheme similar to the method described in Example 88.

MS(M+H)⁺=910.4, ¹H NMR (400 MHz, DMSO-d₆) δ=11.20-10.94 (m, 1H), 8.23 (s, 1H), 8.14 (d, J=8.3 Hz, 1H), 7.99 (s, 1H), 7.67 (d, J=8.5 Hz, 1H), 7.32 (d, J=1.9 Hz, 1H), 7.24 (dd, J=2.1, 8.7 Hz, 1H), 7.04 (d, J=1.6 Hz, 1H), 6.96 (dd, J=1.4, 8.2 Hz, 1H), 5.69-5.57 (m, 2H), 5.06 (dd, J=5.3, 12.9 Hz, 1H), 4.71 (quin, J=8.1 Hz, 1H), 4.02 (br t, J=14.1 Hz, 2H), 3.88 (s, 3H), 3.66-3.45 (m, 4H), 3.43 (br s, 4H), 3.32 (s, 3H), 2.98 (br s, 4H), 2.93-2.83 (m, 1H), 2.62-2.54 (m, 2H), 2.38 (br s, 8H), 2.07-1.97 (m, 1H), 1.95-1.83 (m, 2H), 1.68 (br s, 2H), 1.62-1.49 (m, 4H).

### Example 93. Synthesis of 4-(4-((1-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperidin-4-yl)methyl)piperazin-1-yl)-2-(2,6-dioxopiperidin-3-yl)isoindoline-1,3-dione (Compound 93)

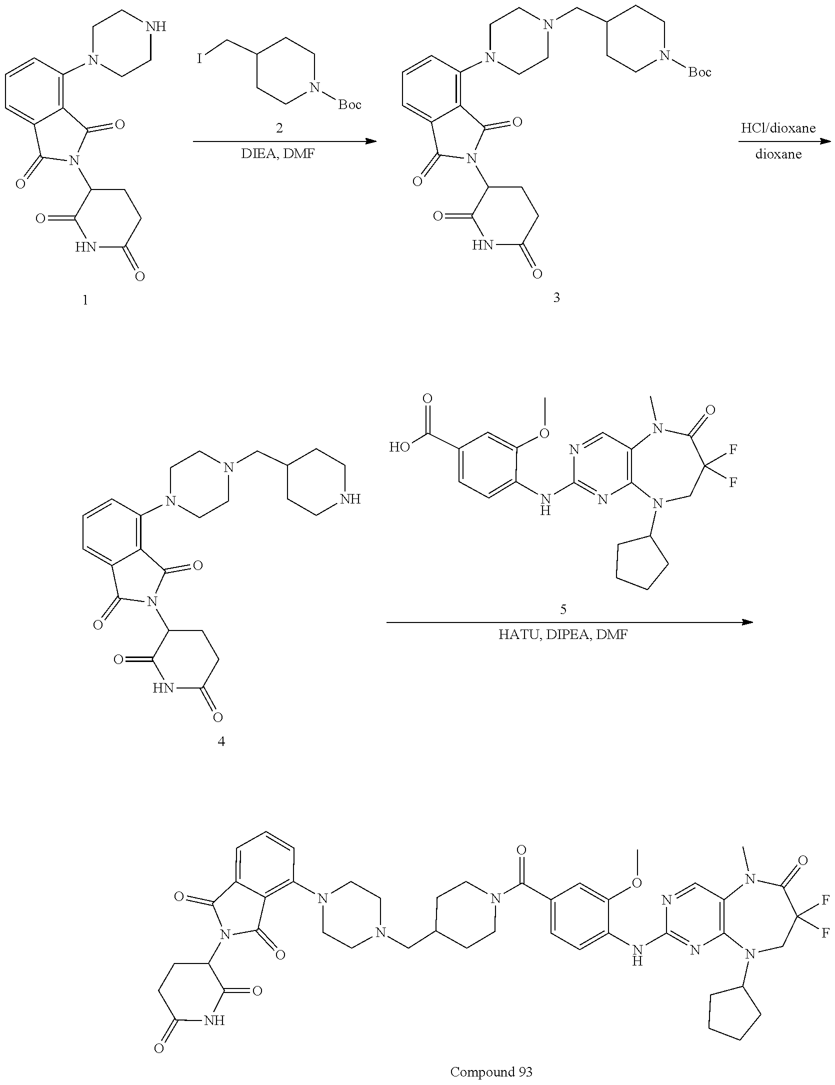

The compound 93 was synthesized by the method described in the scheme similar to the method described in Example 89.

MS(M+H)⁺=869.2, ¹H NMR (400 MHz, DMSO-d₆) δ=11.13-11.05 (m, 1H), 8.24 (s, 1H), 8.13 (d, J=8.2 Hz, 1H), 8.00 (s, 1H), 7.73-7.68 (m, 1H), 7.38-7.31 (m, 2H), 7.04-7.01 (m, 1H), 6.98-6.93 (m, 1H), 5.13-5.06 (m, 1H), 4.76-4.66 (m, 1H), 4.03 (t, J=14.1 Hz, 2H), 3.88 (s, 3H), 3.33-3.26 (m, 7H), 2.93-2.82 (m, 2H), 2.63-2.53 (m, 7H), 2.27-2.21 (m, 2H), 2.07-1.98 (m, 1H), 1.96-1.49 (m, 13H), 1.18-1.03 (m, 2H).

### Example 94. Synthesis of 4-(4-(2-(1-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperidin-4-yl)ethyl)piperazin-1-yl)-2-(2,6-dioxopiperidin-3-yl)isoindoline-1,3-dione (Compound 94)

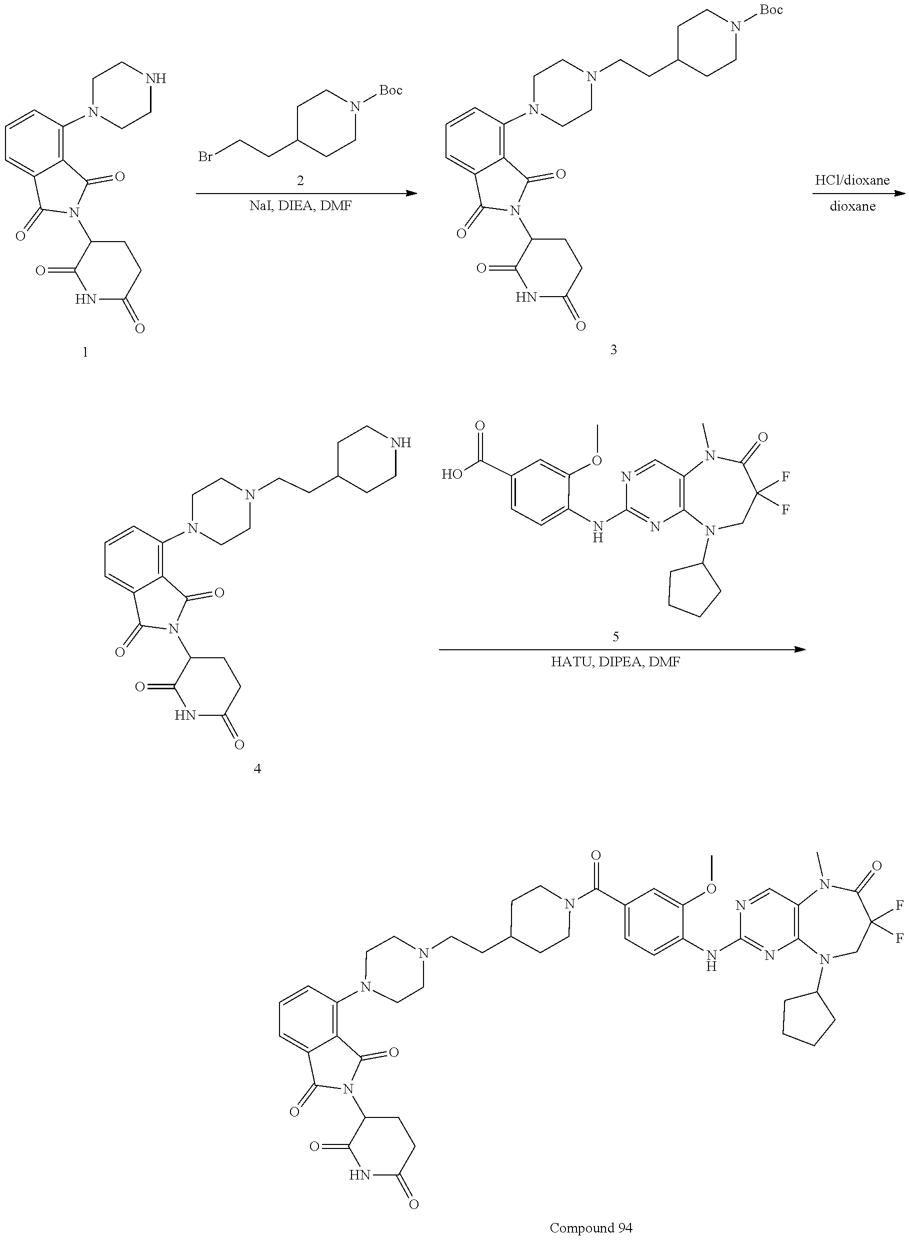

The compound 94 was synthesized by the method described in the scheme similar to the method described in Example 89.

MS(M+H)⁺=883.1, ¹H NMR (400 MHz, DMSO-d₆) δ=11.08 (s, 1H), 8.23 (s, 1H), 8.13 (d, J=8.0 Hz, 1H), 7.99 (s, 1H), 7.70 (t, J=7.8 Hz, 1H), 7.34 (t, J=8.2 Hz, 2H), 7.02 (s, 1H), 6.96-6.92 (m, 1H), 5.12-5.05 (m, 1H), 4.76-4.66 (m, 1H), 4.07-3.97 (m, 2H), 3.88 (s, 3H), 3.29-3.25 (m, 7H), 2.93-2.80 (m, 2H), 2.63-2.52 (m, 7H), 2.41-2.35 (m, 2H), 2.06-1.98 (m, 1H), 1.94-1.85 (m, 2H), 1.75-1.41 (m, 13H), 1.20-1.07 (m, 2H).

### Example 95. Synthesis of 4-(4-(3-(1-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperidin-4-yl)propyl)piperazin-1-yl)-2-(2,6-dioxopiperidin-3-yl)isoindoline-1,3-dione (Compound 95)

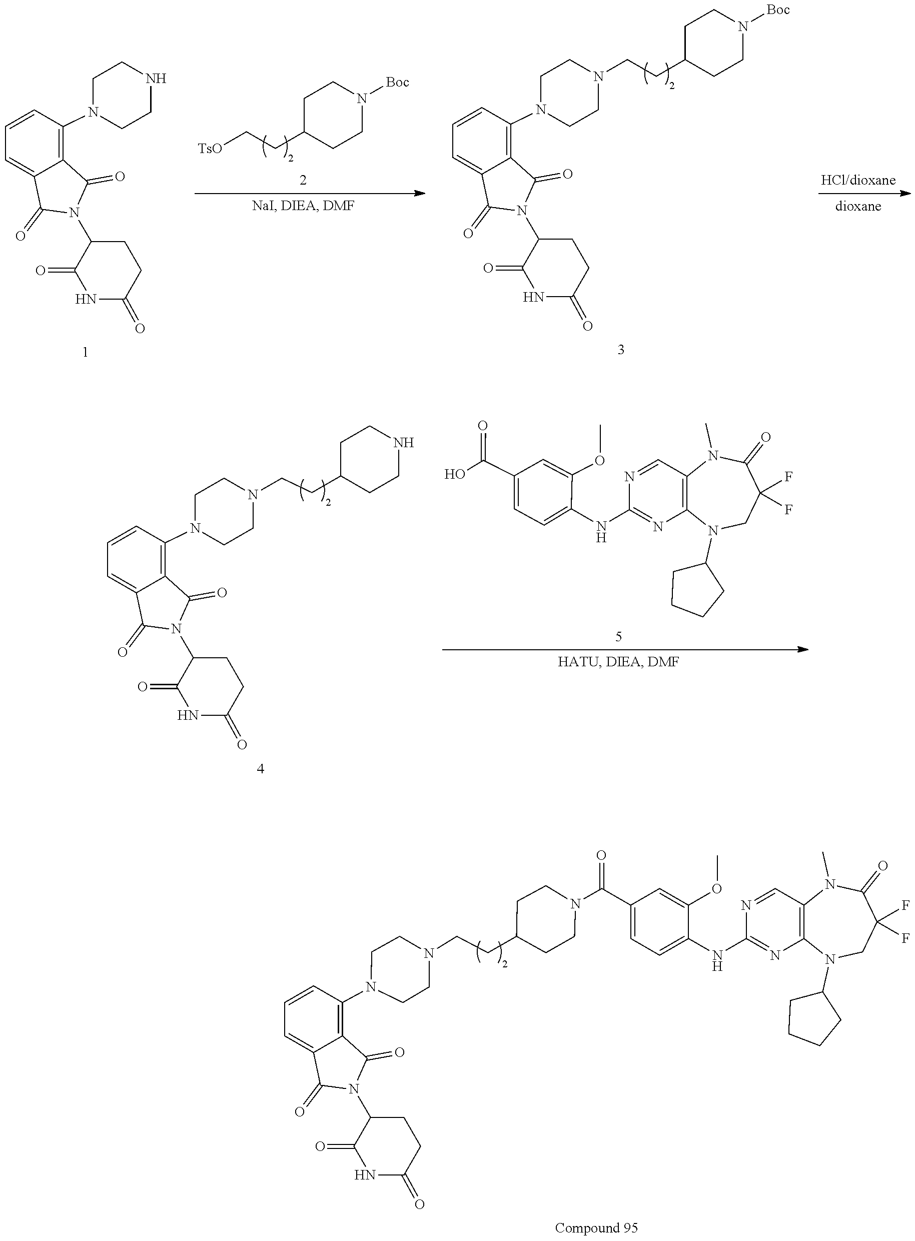

The compound 95 was synthesized by the method described in the scheme similar to the method described in Example 89.

MS(M+H)⁺=897.2, ¹H NMR (400 MHz, DMSO-d₆) δ=11.07 (s, 1H), 8.23 (s, 1H), 8.12 (d, J=8.2 Hz, 1H), 7.98 (s, 1H), 7.72-7.66 (m, 1H), 7.34 (t, J=8.1 Hz, 2H), 7.03-7.00 (d, J=1.5 Hz, 1H), 6.96-6.92 (m, 1H), 5.11-5.05 (m, 1H), 4.76-4.65 (m, 1H), 4.07-3.96 (m, 2H), 3.87 (s, 3H), 3.30-3.25 (m, 7H), 2.92-2.80 (m, 2H), 2.62-2.51 (m, 7H), 2.36-2.29 (m, 2H), 2.06-1.97 (m, 1H), 1.95-1.84 (m, 2H), 1.75-1.44 (m, 13H), 1.31-1.21 (m, 2H), 1.15-1.02 (m, 2H).

### Example 96. Synthesis of 4-(4-(4-(1-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperidin-4-yl)butyl)piperazin-1-yl)-2-(2,6-dioxopiperidin-3-yl)isoindoline-1,3-dione (Compound 96)

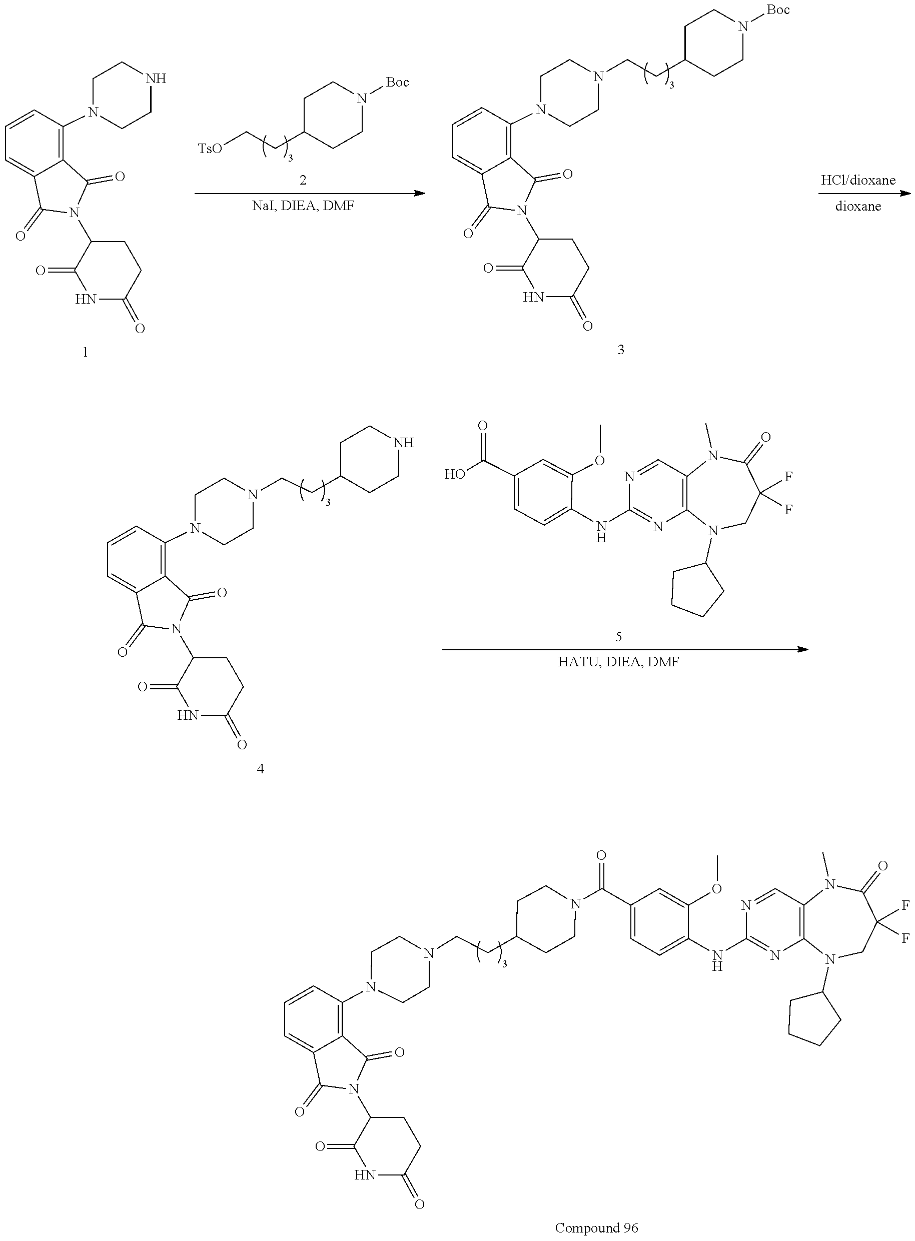

The compound 96 was synthesized by the method described in the scheme similar to the method described in Example 89.

MS(M+H)⁺=911.3, ¹H NMR (400 MHz, DMSO-d₆) δ=11.07 (s, 1H), 8.22 (s, 1H), 8.11 (d, J=8.1 Hz, 1H), 7.98 (s, 1H), 7.71-7.66 (m, 1H), 7.36-7.30 (m, 2H), 7.02-7.00 (m, 1H), 6.95-6.91 (m, 1H), 5.11-5.03 (m, 1H), 4.74-4.65 (m, 1H), 4.06-3.96 (m, 2H), 3.87 (s, 3H), 3.30-3.25 (m, 7H), 2.93-2.80 (m, 2H), 2.62-2.53 (m, 7H), 2.34-2.29 (m, 2H), 2.05-1.96 (m, 1H), 1.95-1.82 (m, 2H), 1.76-1.40 (m, 13H), 1.37-1.20 (m, 4H), 1.14-1.00 (m, 2H).

### Example 97. Synthesis of 5-(4-((1-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperidin-4-yl)methyl)piperazin-1-yl)-2-(2,6-dioxopiperidin-3-yl)isoindoline-1,3-dione (Compound 97)

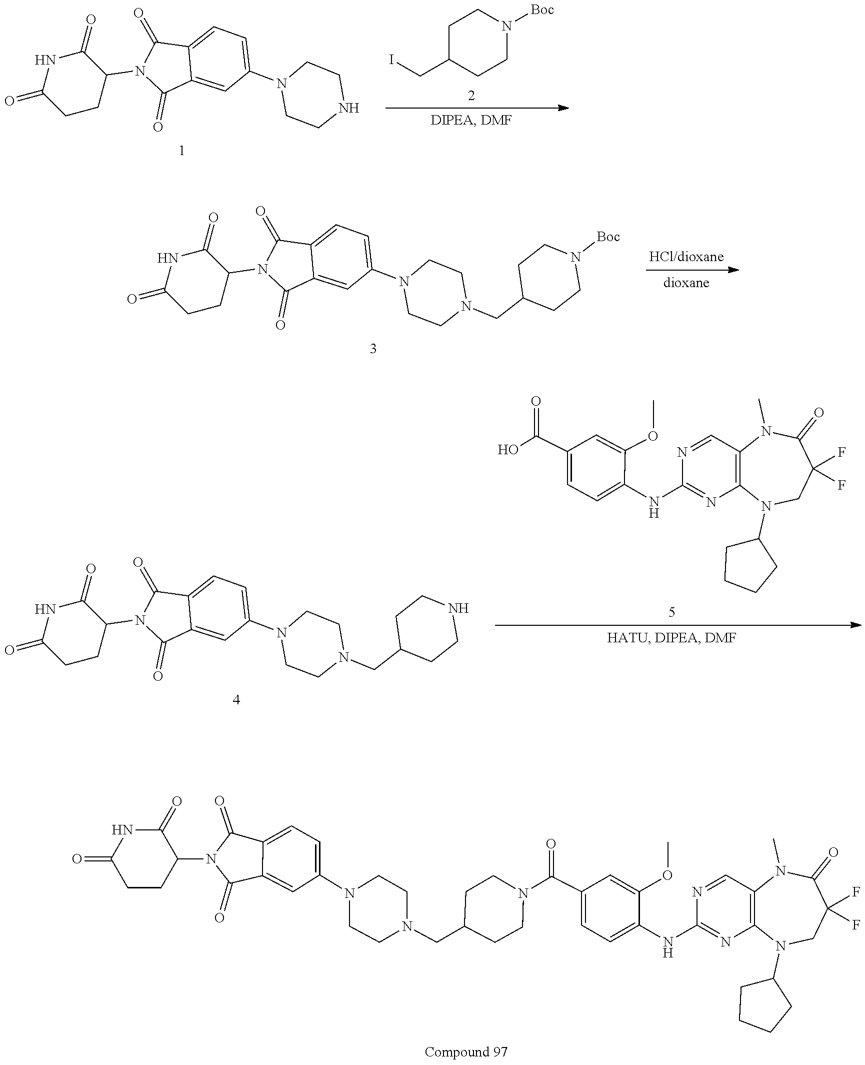

The compound 97 was synthesized by the method described in the scheme similar to the method described in Example 89.

MS(M+H)⁺=869.1, ¹H NMR (400 MHz, DMSO-d₆) δ=11.08 (s, 1H), 8.23 (s, 1H), 8.15-8.11 (m, 1H), 7.99 (s, 1H), 7.69 (d, J=8.3 Hz, 1H), 7.36 (s, 1H), 7.26 (d, J=7.5 Hz, 1H), 7.02 (s, 1H), 6.98-6.92 (m, 1H), 5.07 (dd, J=5.4, 12.8 Hz, 1H), 4.76-4.65 (m, 1H), 4.02 (t, J=14.1 Hz, 2H), 3.88 (s, 3H), 3.53-3.39 (m, 4H), 3.33 (s, 3H), 2.94-2.83 (m, 2H), 2.63-2.51 (m, 7H), 2.31-2.09 (m, 2H), 2.04-1.97 (m, 1H), 1.96-1.46 (m, 13H), 1.18-1.05 (m, 2H).

### Example 98. Synthesis of 5-(4-(2-(1-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperidin-4-yl)ethyl)piperazin-1-yl)-2-(2,6-dioxopiperidin-3-yl)isoindoline-1,3-dione (Compound 98)

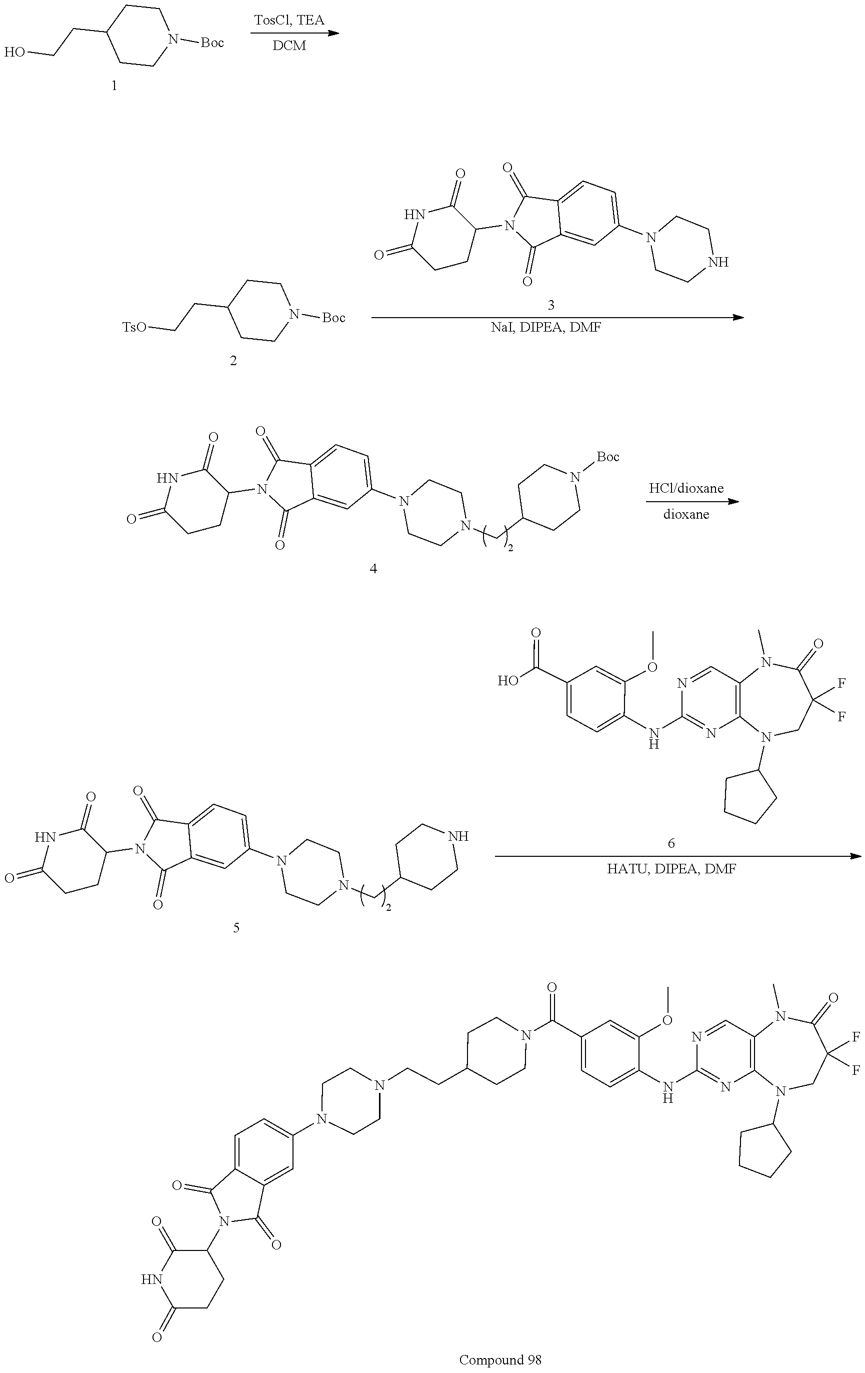

The compound 98 was synthesized by the method described in the scheme similar to the method described in Example 89.

MS(M+H)⁺=883.2, ¹H NMR (400 MHz, DMSO-d₆) δ=11.09 (s, 1H), 8.23 (s, 1H), 8.15-8.11 (m, 1H), 8.01-7.98 (m, 1H), 7.70 (d, J=8.4 Hz, 1H), 7.38 (s, 1H), 7.28 (d, J=8.4 Hz, 1H), 7.02 (d, J=1.5 Hz, 1H), 6.95 (dd, J=1.4, 8.2 Hz, 1H), 5.08 (dd, J=5.3, 12.8 Hz, 1H), 4.76-4.65 (m, 1H), 4.02 (t, J=14.1 Hz, 2H), 3.88 (s, 3H), 3.57-3.41 (m, 4H), 3.32 (s, 3H), 2.93-2.82 (m, 2H), 2.63-2.51 (m, 7H), 2.27-1.83 (m, 5H), 1.80-1.43 (m, 13H), 1.20-1.09 (m, 2H).

### Example 99. Synthesis of 5-(4-(3-(1-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperidin-4-yl)propyl)piperazin-1-yl)-2-(2,6-dioxopiperidin-3-yl)isoindoline-1,3-dione (Compound 99)

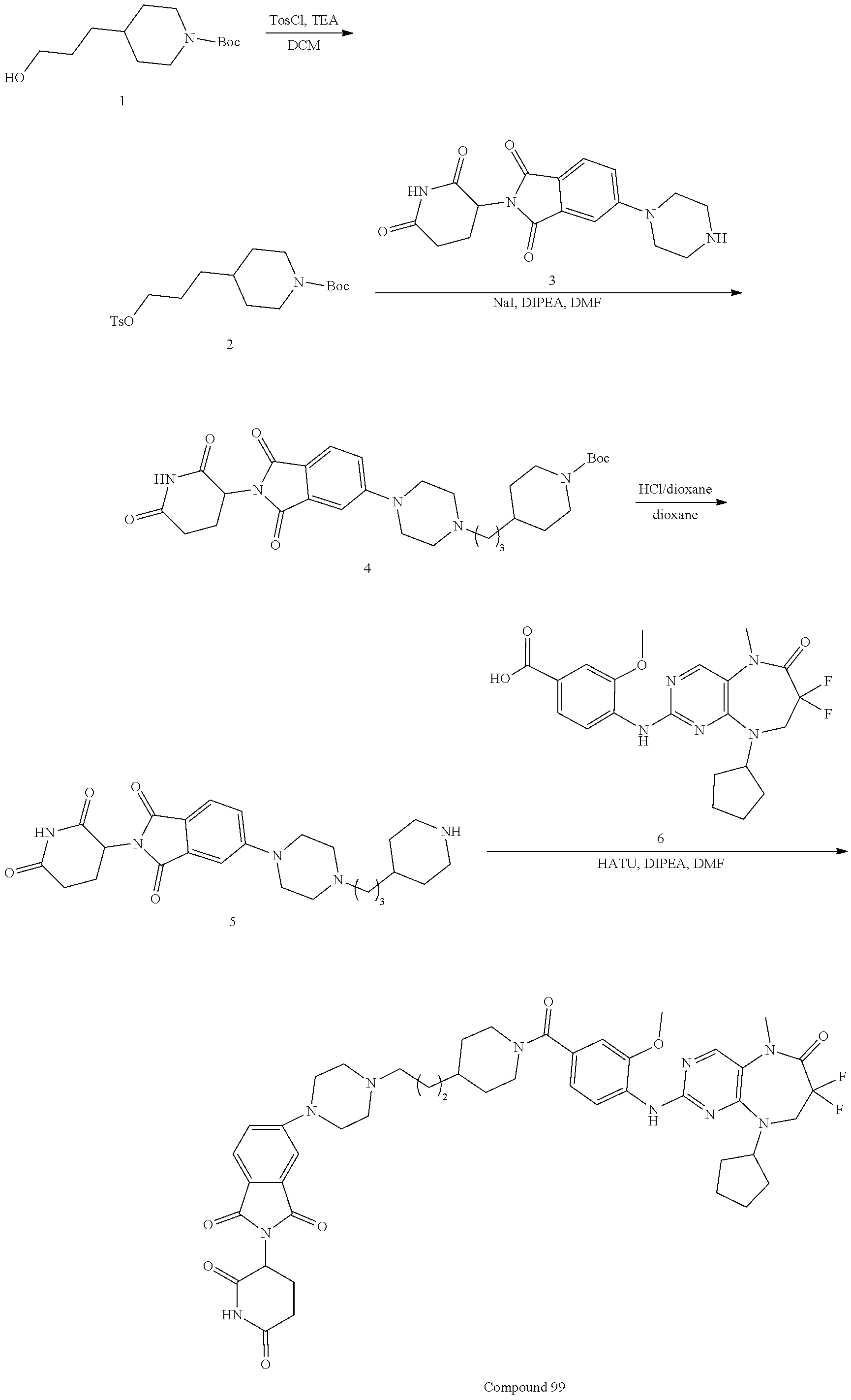

The compound 99 was synthesized by the method described in the scheme similar to the method described in Example 89.

MS(M+H)⁺=897.0, ¹H NMR (400 MHz, DMSO-d₆) δ=11.09 (s, 1H), 8.23 (s, 1H), 8.15-8.11 (m, 1H), 7.99 (s, 1H), 7.68 (d, J=8.6 Hz, 1H), 7.34 (d, J=1.6 Hz, 1H), 7.25 (dd, J=2.0, 8.7 Hz, 1H), 7.02 (d, J=1.5 Hz, 1H), 6.94 (dd, J=1.5, 8.1 Hz, 1H), 5.07 (dd, J=5.4, 12.9 Hz, 1H), 4.76-4.65 (m, 1H), 4.02 (t, J=14.1 Hz, 2H), 3.88 (s, 3H), 4.00-3.42 (m, 4H), 3.32 (s, 3H), 2.94-2.82 (m, 2H), 2.63-2.51 (m, 7H), 2.32 (t, J=6.0 Hz, 2H), 2.05-1.97 (m, 1H), 1.94-1.86 (m, 2H), 1.81-1.40 (m, 13H), 1.31-1.21 (m, 2H), 1.10-1.01 (m, 2H).

### Example 100. Synthesis of 5-(4-(4-(1-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperidin-4-yl)butyl)piperazin-1-yl)-2-(2,6-dioxopiperidin-3-yl)isoindoline-1,3-dione (Compound 100)

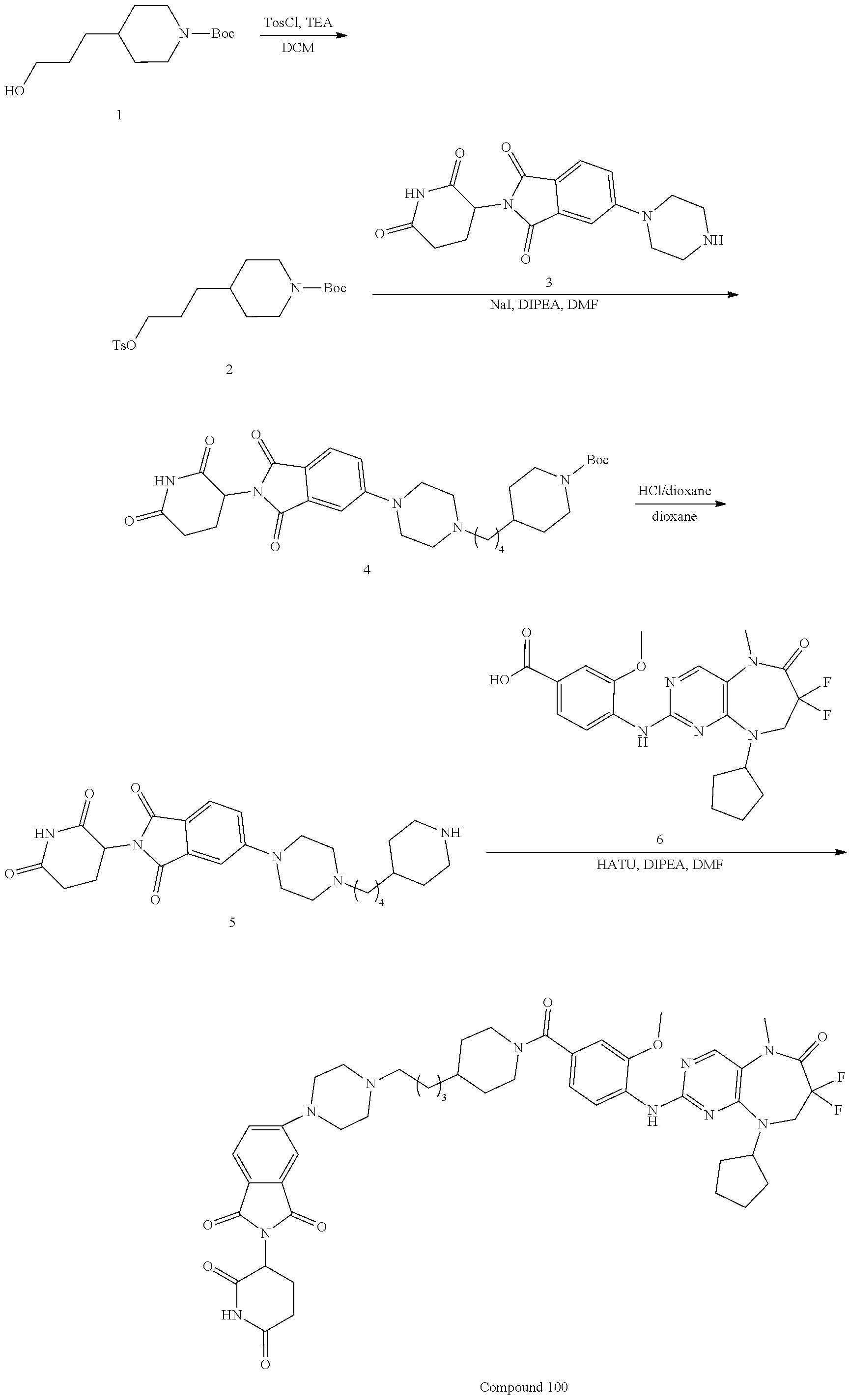

The compound 100 was synthesized by the method described in the scheme similar to the method described in Example 89.

MS(M+H)⁺=911.2, ¹H NMR (400 MHz, DMSO-d₆) δ=11.07 (br s, 1H), 8.29-8.23 (m, 1H), 8.12 (d, J=8.2 Hz, 1H), 7.98 (s, 1H), 7.67 (d, J=8.6 Hz, 1H), 7.33 (s, 1H), 7.25 (dd, J=1.7, 8.7 Hz, 1H), 7.02 (d, J=1.2 Hz, 1H), 6.94 (d, J=8.2 Hz, 1H), 5.07 (dd, J=5.3, 12.9 Hz, 1H), 4.77-4.65 (m, 1H), 4.02 (t, J=14.2 Hz, 2H), 3.87 (s, 3H), 3.42-3.40 (m, 4H), 3.32 (s, 3H), 2.94-2.82 (m, 2H), 2.62-2.51 (m, 5H), 2.48-2.47 (m, 2H), 2.31 (t, J=7.1 Hz, 2H), 2.05-1.97 (m, 1H), 1.94-1.85 (m, 2H), 1.79-1.39 (m, 13H), 1.37-1.24 (m, 4H), 1.16-1.01 (m, 2H).

### Example 101. Synthesis of 5-(4-((1-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperidin-4-yl)methyl)piperazin-1-yl)-2-(2,6-dioxopiperidin-3-yl)-6-fluoroisoindoline-1,3-dione (Compound 101)

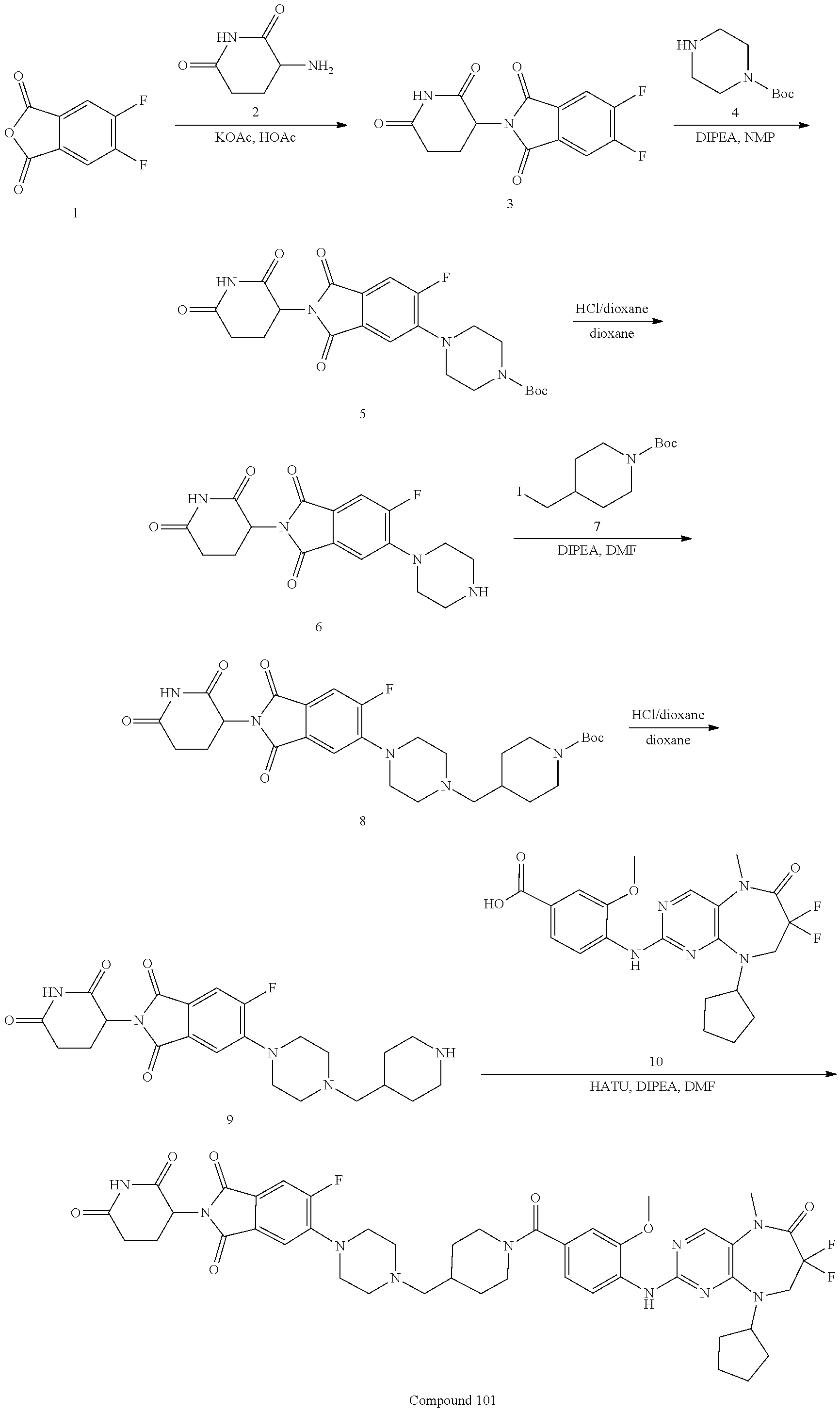

### Step 1. Synthesis of 2-(2,6-dioxopiperidin-3-yl)-5,6-difluoroisoindoline-1,3-dione (3)

To the solution of 5,6-difluoroisobenzofuran-1,3-dione (10 g, 54.32 mmol) and 3-aminopiperidine-2,6-dione (9.39 g, 57.04 mmol, HCl salt) in AcOH (200 mL) was added KOAc (5.86 g, 59.75 mmol) and the resulting mixture was stirred at 90° C. for 12 h. LCMS showed that the reaction was completed, the reaction mixture was poured into water (1000 mL) and the resulting suspension was filtered, the filter cake was collected and dried in vacuo to afford 2-(2,6-dioxopiperidin-3-yl)-5,6-difluoroisoindoline-1,3-dione (13 g, 43.74 mmol, 80.53% yield, 99% purity) as a brown solid. MS(M+H)⁺=295.1

### Step 2. Synthesis of tert-butyl 4-(2-(2,6-dioxopiperidin-3-yl)-6-fluoro-1,3-dioxoisoindolin-5-yl)piperazine-1-carboxylate (5)

To the solution of 2-(2,6-dioxopiperidin-3-yl)-5,6-difluoroisoindoline-1,3-dione (3 g, 10.20 mmol) and tert-butyl piperazine-1-carboxylate (2.09 g, 11.22 mmol) in NMP (30 mL) was added DIPEA (3.95 g, 30.59 mmol, 5.33 mL) and the resulting mixture was stirred at 110° C. for 12 h. LCMS showed completion of reaction. The mixture was poured into water (100 mL) and extracted with EtOAc (50 mL×3). The combined organic layer was washed with brine (100 mL), dried over Na₂SO₄, filtered and concentrated to afford tert-butyl 4-(2-(2,6-dioxopiperidin-3-yl)-6-fluoro-1,3-dioxoisoindolin-5-yl)piperazine-1-carboxylate (4.3 g, 9.15 mmol, 89.75% yield, 98% purity) as yellow oil. MS(M−55+H)⁺=405.0

### Step 3. Synthesis of 2-(2,6-dioxopiperidin-3-yl)-5-fluoro-6-(piperazin-1-yl)isoindoline-1,3-dione (6)

To the solution of tert-butyl 4-(2-(2,6-dioxopiperidin-3-yl)-6-fluoro-1,3-dioxoisoindolin-5-yl)piperazine-1-carboxylate (4.3 g, 9.34 mmol) in dioxane (20 mL) was added HCl/dioxane (4 M, 20 mL) and the resulting mixture was stirred at 20° C. for 12 h. LCMS showed completion of reaction, the mixture was concentrated to afford 2-(2,6-dioxopiperidin-3-yl)-5-fluoro-6-(piperazin-1-yl)isoindoline-1,3-dione (3.5 g, 8.11 mmol, 86.90% yield, 92% purity, HCl salt) as a yellow solid. MS(M+H)⁺=360.8

### Step 4. Synthesis of tert-butyl 4-((4-(2-(2,6-dioxopiperidin-3-yl)-6-fluoro-1,3-dioxoisoindolin-5-yl)piperazin-1-yl) methyl)piperidine-1-carboxylate (8)

To the solution of 2-(2,6-dioxopiperidin-3-yl)-5-fluoro-6-(piperazin-1-yl)isoindoline-1,3-dione (2 g, 5.04 mmol, HCl salt) and tert-butyl 4-(iodomethyl) piperidine-1-carboxylate (1.64 g, 5.04 mmol) in DMF (50 mL) was added DIPEA (1.95 g, 15.12 mmol, 2.63 mL) and the resulting mixture was stirred at 60° C. for 12 h. LCMS showed completion of reaction, the mixture was poured into water (300 mL) and extracted with EtOAc (100 mL×3). The combined organic layer was washed with brine (200 mL), dried over Na₂SO₄, filtered and concentrated. The residue was purified by flash silica gel chromatography (ISCO®; 50 g Sepa Flash® Silica Flash Column, Eluent of 0˜10% MeOH/EtOAc gradient @80 mL/min) to afford tert-butyl 4-((4-(2-(2,6-dioxopiperidin-3-yl)-6-fluoro-1,3-dioxoisoindolin-5-yl)piperazin-1-yl)methyl)piperidine-1-carboxylate (1 g, 1.79 mmol, 35.58% yield) as yellow oil. MS(M+H)⁺=558.3

¹H NMR (400 MHz, CDCl₃) δ=8.38 (s, 1H), 7.47 (d, J=11.0 Hz, 1H), 7.39 (br d, J=7.1 Hz, 1H), 4.98-4.85 (m, 1H), 4.23-4.02 (m, 2H), 3.45-3.22 (m, 4H), 2.87-2.56 (m, 10H), 2.41-2.23 (m, 2H), 2.17-2.10 (m, 1H), 1.88-1.86 (m, 2H), 1.46 (s, 9H), 1.19-1.04 (m, 2H).

### Step 5. Synthesis of 2-(2,6-dioxopiperidin-3-yl)-5-fluoro-6-(4-(piperidin-4-ylmethyl)piperazin-1-yl)isoindoline-1,3-dione (9)

To the solution of tert-butyl 4-((4-(2-(2,6-dioxopiperidin-3-yl)-6-fluoro-1,3-dioxoisoindolin-5-yl)piperazin-1-yl)methyl)piperidine-1-carboxylate (1 g, 1.79 mmol) in dioxane (10 mL) was added HCl/dioxane (4 M, 10.00 mL) and the resulting mixture was stirred at 20° C. for 12 h. LCMS showed completion of reaction. The reaction mixture was concentrated to afford 2-(2,6-dioxopiperidin-3-yl)-5-fluoro-6-(4-(piperidin-4-ylmethyl)piperazin-1-yl)isoindoline-1,3-dione (880 mg, 1.78 mmol, 99.34% yield, HCl salt) as a yellow solid. MS(M+H)⁺=458.2

### Step 6. Synthesis of 5-(4-((1-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperidin-4-yl)methyl)piperazin-1-yl)-2-(2,6-dioxopiperidin-3-yl)-6-fluoroisoindoline-1,3-dione (Compound 101)

To the solution of 2-(2,6-dioxopiperidin-3-yl)-5-fluoro-6-(4-(piperidin-4-ylmethyl)piperazin-1-yl)isoindoline-1,3-dione (880 mg, 1.78 mmol, HCl salt) and 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (797.12 mg, 1.78 mmol) in DMF (20 mL) were added HATU (1.02 g, 2.67 mmol) and DIPEA (690.75 mg, 5.34 mmol) and the resulting mixture was stirred at 25° C. for 1 h. LCMS showed completion of reaction, the mixture was poured into water (100 mL) and extracted with EtOAc (50 mL×3). The combined organic layer was washed with brine (100 mL), dried over Na₂SO₄, filtered and concentrated. The residue was purified by flash silica gel chromatography (10 g Sepa Flash® Silica Flash Column, Eluent of 0˜10% MeOH/EtOAc gradient @100 mL/min) followed by prep-HPLC (column: Waters Xbridge C18 150*50 mm*10 um; mobile phase: [water(NH₄HCO₃)-ACN]; B %: 43%-73%, 11 min) and the eluent was lyophilized to afford 5-(4-((1-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)piperidin-4-yl)methyl)piperazin-1-yl)-2-(2,6-dioxopiperidin-3-yl)-6-fluoroisoindoline-1,3-dione (243 mg, 268.50 μmol, 15.07% yield, 98% purity) as a yellow solid. MS(M+H)⁺=887.2

¹H NMR (400 MHz, DMSO-d₆) δ=11.11 (s, 1H), 8.24 (s, 1H), 8.14 (d, J=8.3 Hz, 1H), 8.00 (s, 1H), 7.73 (d, J=11.5 Hz, 1H), 7.46 (d, J=7.6 Hz, 1H), 7.04 (s, 1H), 6.96 (d, J=8.3 Hz, 1H), 5.11 (br dd, J=5.4, 13.0 Hz, 1H), 4.77-4.66 (m, 1H), 4.03 (br t, J=13.9 Hz, 2H), 3.89 (s, 3H), 3.32 (br s, 3H), 3.27-3.25 (m, 4H), 2.95-2.83 (m, 2H), 2.70-2.54 (m, 7H), 2.25-2.23 (m, 2H), 2.10-1.99 (m, 1H), 1.97-1.52 (m, 13H), 1.21-1.03 (m, 2H).

### Example 102. Synthesis of 4-(4-(3-(4-(6-((9-cyclopentyl-7,7-difluoro-5-methyl-6-oxo-6,7,8,9-tetrahydro-5H-pyrimido[4,5-b][1,4]diazepin-2-yl)amino)-7-methoxy-1-oxo-3,4-dihydroisoquinolin-2(1H)-yl)piperidin-1-yl)-3-oxopropyl)piperazin-1-yl)-2-(2,6-dioxopiperidin-3-yl)isoindoline-1,3-dione (Compound 102)

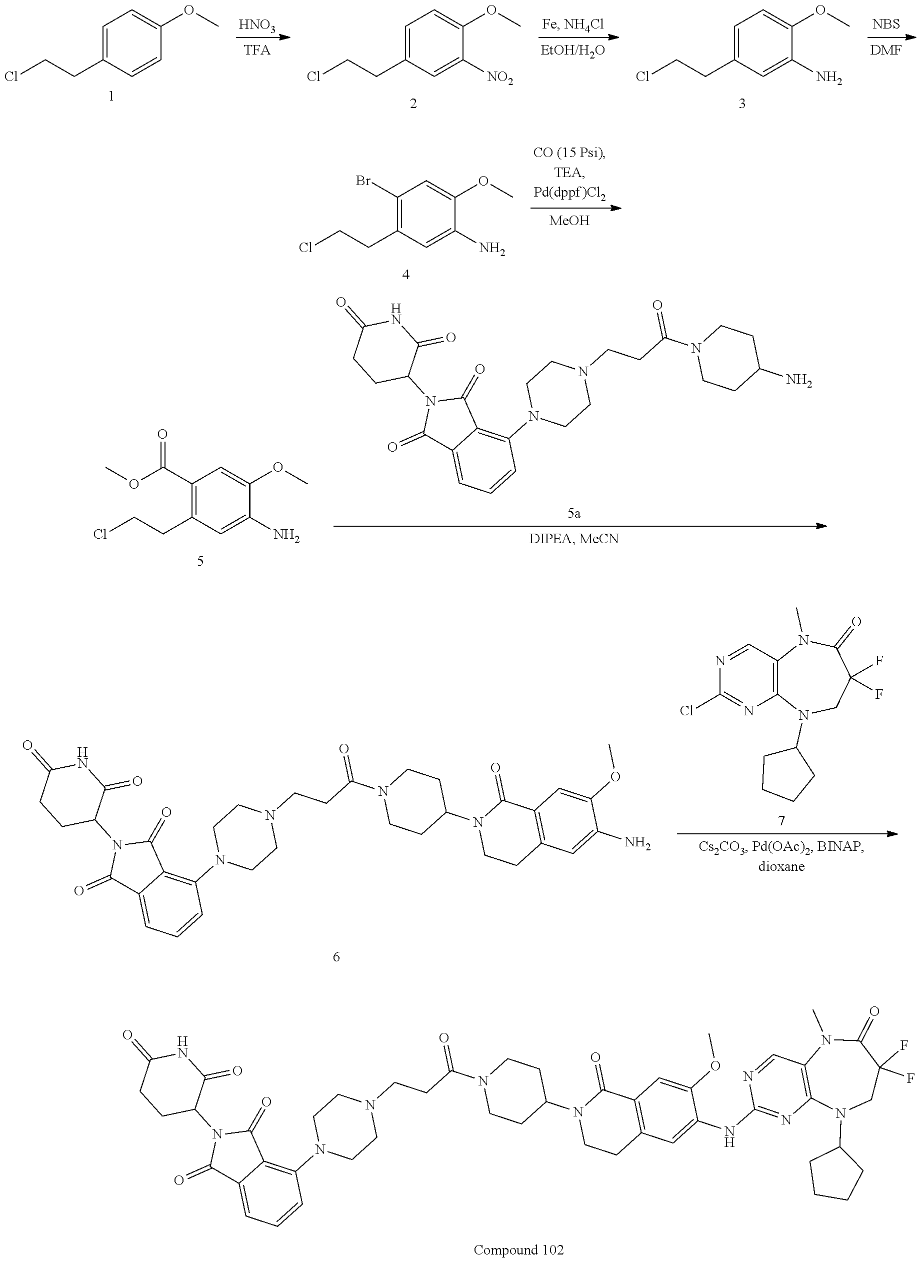

### Step 1. Synthesis of 4-(2-chloroethyl)-1-methoxy-2-nitrobenzene (2)

To a solution of 1-(2-chloroethyl)-4-methoxybenzene (10 g, 58.60 mmol) in TFA (100 mL) was added HNO₃ (6.14 g, 63.38 mmol, 4.39 mL, 65% purity) drop-wise at 0° C. The resulting mixture was stirred at 25° C. for 2 h. TLC (petroleum ether:EtOAc 5:1, Rf=0.4) showed the starting material was consumed completely. The reaction mixture was poured into ice water (400 mL) and extracted with EtOAc (50 mL×4). The combined organic layers were washed with NaHCO₃ (50 mL×3) and brine (50 mL), dried over Na₂SO₄ and concentrated to afford the 4-(2-chloroethyl)-1-methoxy-2-nitro-benzene (11 g, crude) as black brown oil. MS(M+H)⁺=216.6

### Step 2. Synthesis of 5-(2-chloroethyl)-2-methoxyaniline (3)

To a mixture of 4-(2-chloroethyl)-1-methoxy-2-nitrobenzene (9 g, 41.74 mmol) and NH₄Cl (11.16 g, 208.69 mmol) in EtOH (60 mL) and H₂O (30 mL) was added Fe (11.65 g, 208.69 mmol) at 25° C. The resulting mixture was stirred at 80° C. for 1 h. HPLC showed the starting material was consumed completely. The reaction mixture was filtered through celite pad while hot and washed with EtOH (50 mL). The filtrate was concentrated to remove the organic phase. The residue was diluted with water (100 mL), extracted with EtOAc (50 mL×4). The combined organic layers were dried over Na₂SO₄ and concentrated to give the 5-(2-chloroethyl)-2-methoxy-aniline (6.5 g, crude) as black brown oil. MS(M+H)⁺=186.1

### Step 3. Synthesis of 4-bromo-5-(2-chloroethyl)-2-methoxyaniline (4)

To a solution of 5-(2-chloroethyl)-2-methoxyaniline (6.5 g, 35.01 mmol) in DMF (60 mL) was added a solution of NBS (5.61 g, 31.51 mmol) in DMF (20 mL) drop-wise at 0° C. The resulting solution was warmed to 25° C. slowly and stirred for 1 h. LCMS showed the starting material was consumed completely and a main peak with desired mass. The reaction mixture was poured into water (400 mL), then extracted petroleum ether/EtOAc (10/1, 200 mL×3). The combined organic layers were washed with brine (100 mL×2), dried over Na₂SO₄ and concentrated. The residue was purified by flash silica gel chromatography (40 g silica gel column, EtOAc/petroleum ether=0-5%, 100 mL/min) to afford the 4-bromo-5-(2-chloroethyl)-2-methoxyaniline (6 g, 22.68 mmol, 64.78% yield) as yellow oil. MS(M+H)⁺=266.0

### Step 4. Synthesis of methyl 4-amino-2-(2-chloroethyl)-5-methoxybenzoate (5)

To a solution of 4-bromo-5-(2-chloroethyl)-2-methoxyaniline (4.5 g, 17.01 mmol) and TEA (8.61 g, 85.05 mmol, 11.84 mL) in MeOH (80 mL) was added Pd(dppf)Cl₂ (622.32 mg, 850.51 μmol) at 25° C. The resulting mixture was purged and degassed with CO for three times, heated to 70° C. and stirred under CO (15 Psi) for 14 h. LCMS showed the starting material remained and a peak with desired mass. The reaction mixture was and filtered. The filtrate was concentrated to give the crude product. The crude product was purified by flash silica gel chromatography (40 g silica gel column, EtOAc/petroleum ether=0-8%, 80 mL/min) to afford the methyl 4-amino-2-(2-chloroethyl)-5-methoxybenzoate (600 mg, 2.46 mmol, 14.47% yield, N/A purity) as yellow oil. MS(M+H)⁺=244.0

### Step 5. Synthesis of 4-(4-(3-(4-(6-amino-7-methoxy-1-oxo-3,4-dihydroisoquinolin-2(1H)-yl)piperidin-1-yl)-3-oxopropyl)piperazin-1-yl)-2-(2,6-dioxopiperidin-3-yl)isoindoline-1,3-dione (6)

A mixture of 4-(4-(3-(4-aminopiperidin-1-yl)-3-oxopropyl)piperazin-1-yl)-2-(2,6-dioxopiperidin-3-yl)isoindoline-1,3-dione (400 mg, 750.44 μmol, HCl), methyl 4-amino-2-(2-chloroethyl)-5-methoxybenzoate (182.87 mg, 750.44 μmol) and DIPEA (484.95 mg, 3.75 mmol, 653.57 μL) in ACN (8 mL) was stirred at 80° C. for 16 h. LCMS showed the starting material remained and a peak with desired mass. The reaction mixture was stirred at 80° C. for another 16 h. LCMS showed a main peak with desired mass. The reaction mixture was concentrated. The residue was purified by flash silica gel chromatography (4 g silica gel column, EtOAc/petroleum ether=20-100% and then methanol/EtOAc=10-30%, 40 mL/min) to afford the 4-(4-(3-(4-(6-amino-7-methoxy-1-oxo-3,4-dihydroisoquinolin-2(1H)-yl)piperidin-1-yl)-3-oxopropyl)piperazin-1-yl)-2-(2,6-dioxopiperidin-3-yl)isoindoline-1,3-dione (430 mg, 640.13 μmol, 85.30% yield, 100% purity) as yellow solid. MS(M+H)⁺=672.3

### Step 6. Synthesis of 4-(4-(3-(4-(6-((9-cyclopentyl-7,7-difluoro-5-methyl-6-oxo-6,7,8,9-tetrahydro-5H-pyrimido[4,5-b][1,4]diazepin-2-yl)amino)-7-methoxy-1-oxo-3,4-dihydroisoquinolin-2(1H)-yl)piperidin-1-yl)-3-oxopropyl)piperazin-1-yl)-2-(2,6-dioxopiperidin-3-yl)isoindoline-1,3-dione (Compound 102)

To a solution of 4-(4-(3-(4-(6-amino-7-methoxy-1-oxo-3,4-dihydroisoquinolin-2(1H)-yl)piperidin-1-yl)-3-oxopropyl)piperazin-1-yl)-2-(2,6-dioxopiperidin-3-yl)isoindoline-1,3-dione (200 mg, 297.73 μmol), 2-chloro-9-cyclopentyl-7,7-difluoro-5-methyl-5,7,8,9-tetrahydro-6H-pyrimido[4,5-b][1,4]diazepin-6-one (103.73 mg, 327.51 μmol) in dioxane (3 mL) were added Cs₂CO₃ (291.02 mg, 893.20 μmol), BINAP (92.69 mg, 148.87 μmol), Pd(OAc)₂ (16.71 mg, 74.43 μmol) at 25° C. The mixture was stirred at 100° C. for 16 h under N₂ atmosphere. LCMS showed the 4-(4-(3-(4-(6-amino-7-methoxy-1-oxo-3,4-dihydroisoquinolin-2(1H)-yl)piperidin-1-yl)-3-oxopropyl)piperazin-1-yl)-2-(2,6-dioxopiperidin-3-yl)isoindoline-1,3-dione was consumed completely, and a peak (29%) with desired mass. The mixture solution was concentrated under reduced pressure. The residue was purified by prep-HPLC (column: Phenomenex Synergi C18 150×25 mm×10 um; mobile phase: [water(FA)-ACN]; B %: 9%-39%, 10 min) and the eluent was lyophilized. The residue was re-purified by prep-HPLC (column: Waters Xbridge BEH C18 150×25 mm×5 um; mobile phase: [water(NH₄HCO₃)-ACN]; B %: 50%-70%, min) and the eluent was lyophilized to afford 4-(4-(3-(4-(6-((9-cyclopentyl-7,7-difluoro-5-methyl-6-oxo-6,7,8,9-tetrahydro-5H-pyrimido[4,5-b][1,4]diazepin-2-yl)amino)-7-methoxy-1-oxo-3,4-dihydroisoquinolin-2(1H)-yl)piperidin-1-yl)-3-oxopropyl)piperazin-1-yl)-2-(2,6-dioxopiperidin-3-yl)isoindoline-1,3-dione (12.9 mg, 12.74 μmol, 4.28% yield, 94% purity) as yellow solid. MS (M+H)⁺=952.5

¹H NMR (400 MHz, DMSO-d₆) δ=11.43-10.80 (m, 1H), 8.27 (s, 1H), 8.16 (s, 1H), 7.95 (s, 1H), 7.70 (dd, J=7.3, 8.4 Hz, 1H), 7.51 (s, 1H), 7.39-7.31 (m, 2H), 5.09 (dd, J=5.4, 12.8 Hz, 1H), 4.83 (quin, J=8.4 Hz, 1H), 4.30 (t, J=5.7 Hz, 2H), 4.23-4.13 (m, 1H), 4.11-3.94 (m, 3H), 3.92-3.83 (m, 4H), 3.31-3.17 (m, 7H), 2.99-2.80 (m, 5H), 2.76-2.72 (m, 1H), 2.68-2.60 (m, 5H), 2.59-2.52 (m, 2H), 2.06-1.91 (m, 3H), 1.82-1.25 (m, 12H).

### Example 103. Synthesis of 4-(4-((1-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxyphenyl)piperidin-4-yl)methyl)piperazin-1-yl)-2-(2,6-dioxopiperidin-3-yl)isoindoline-1,3-dione (Compound 103)

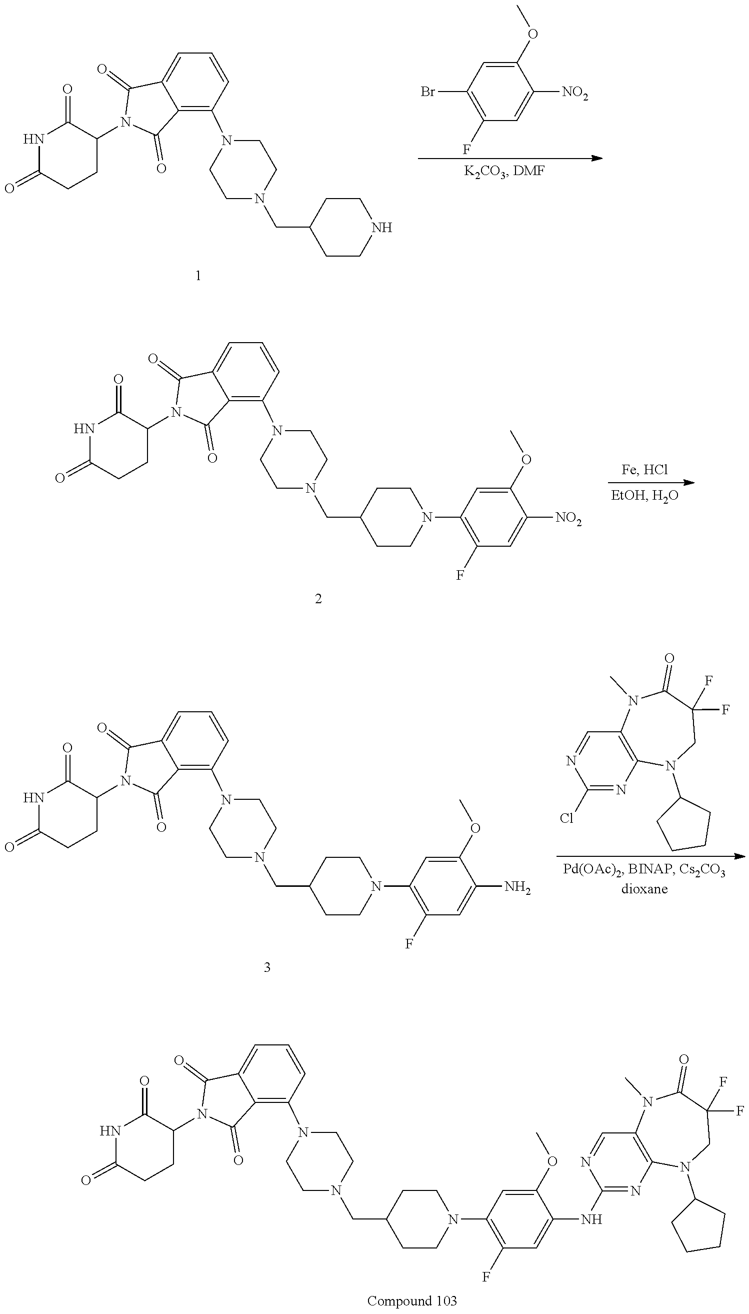

### Step 1. Synthesis of 2-(2,6-dioxopiperidin-3-yl)-4-(4-((1-(2-fluoro-5-methoxy-4-nitrophenyl)piperidin-4-yl)methyl)piperazin-1-yl)isoindoline-1,3-dione (2)

To a solution of 2-(2,6-dioxopiperidin-3-yl)-4-(4-(piperidin-4-ylmethyl)piperazin-1-yl)isoindoline-1,3-dione (1.3 g, 2.73 mmol, HCl salt) and 1-bromo-2-fluoro-5-methoxy-4-nitrobenzene (614 mg, 2.46 mmol) in DMF (15 mL) was added K₂CO₃ (1.13 g, 8.19 mmol) and the mixture was stirred at 80° C. for 14 h. LCMS showed a peak (49%) with desired mass. The mixture was filtered and the filter cake was washed with EtOAc (20 mL) and H₂O (20 mL). The filtrate was extracted with EtOAc (15 mL×3), the combined organic layer was washed with brine (10 mL×3), concentrated under reduced pressure. The crude was diluted with EtOAc (20 mL) and stirred at 20° C. for 1 h. The mixture was filtered and the filter cake was washed with EtOAc (10 mL). The filter cake was collected and dried in vacuo to afford 2-(2,6-dioxopiperidin-3-yl)-4-(4-((1-(2-fluoro-5-methoxy-4-nitrophenyl)piperidin-4-yl)methyl)piperazin-1-yl)isoindoline-1,3-dione (520 mg, 786.05 μmol, 28.78% yield, 92% purity) as a yellow solid. MS(M+H)⁺=609.1

### Step 2. Synthesis of 4-(4-((1-(4-amino-2-fluoro-5-methoxyphenyl)piperidin-4-yl)methyl)piperazin-1-yl)-2-(2,6-dioxopiperidin-3-yl)isoindoline-1,3-dione (3)

To a solution of 2-(2,6-dioxopiperidin-3-yl)-4-(4-((1-(2-fluoro-5-methoxy-4-nitrophenyl)piperidin-4-yl)methyl)piperazin-1-yl)isoindoline-1,3-dione (520 mg, 854.40 μmol) in EtOH (10 mL) and H₂O (10 mL) was added Fe (286.28 mg, 5.13 mmol) and HCl (12 M, 420 μL) and the mixture was stirred at 80° C. for 1 h. LCMS showed the starting material was consumed and desired mass was detected. The mixture was diluted with DMF (20 mL) and H₂O (40 mL), then adjusted the pH=8 with saturated NaHCO₃ at 0° C. The mixture was extracted with EtOAc (20 mL×3) and washed with brine (10 mL×2), dried over Na₂SO₄, filtered and concentrated under reduced pressure to afford 4-(4-((1-(4-amino-2-fluoro-5-methoxyphenyl)piperidin-4-yl)methyl)piperazin-1-yl)-2-(2,6-dioxopiperidin-3-yl)isoindoline-1,3-dione (240 mg, crude) as a yellow solid. MS(M+H)⁺=579.3

### Step 3. Synthesis of 4-(4-((1-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxyphenyl)piperidin-4-yl)methyl)piperazin-1-yl)-2-(2,6-dioxopiperidin-3-yl)isoindoline-1,3-dione (Compound 103)

To a solution of 4-(4-((1-(4-amino-2-fluoro-5-methoxyphenyl)piperidin-4-yl)methyl)piperazin-1-yl)-2-(2,6-dioxopiperidin-3-yl)isoindoline-1,3-dione (240 mg, 414.77 μmol) and 2-chloro-9-cyclopentyl-7,7-difluoro-5-methyl-5,7,8,9-tetrahydro-6H-pyrimido[4,5-b][1,4]diazepin-6-one (120.00 mg, 378.87 μmol) in dioxane (6 mL) were added Pd(OAc)₂ (4.66 mg, 20.74 μmol), BINAP (25.83 mg, 41.48 μmol) and Cs₂CO₃ (405.42 mg, 1.24 mmol) and the mixture was stirred at 100° C. for 14 h. LCMS showed a peak (22%) with desired mass and 36% of reagent 1 remained. Additional 2-chloro-9-cyclopentyl-7,7-difluoro-5-methyl-5,7,8,9-tetrahydro-6H-pyrimido[4,5-b][1,4]diazepin-6-one (120 mg, 378.87 μmol), Pd(OAc)₂ (4.66 mg, 20.74 μmol) and BINAP (25.83 mg, 41.48 μmol) were added and the mixture was stirred at 100° C. for 14 h. LCMS showed a peak (28%) with desired mass. The mixture was diluted with H₂O (10 mL) and extracted with EtOAc (10 mL×3). The combined organic layer was washed with brine (10 mL×3), dried over Na₂SO₄ and filtered. The filtrate was concentrated under reduced pressure. The crude was purified by prep-TLC (Dichloromethane:Methanol=20/1) and then purified by prep-HPLC (column: Waters Xbridge 150*25 mm*5 um; mobile phase: [water(NH₄HCO₃)-ACN]; B %: 60%-90%, 10 min) and the eluent was lyophilized to afford 4-(4-((1-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxyphenyl)piperidin-4-yl)methyl)piperazin-1-yl)-2-(2,6-dioxopiperidin-3-yl)isoindoline-1,3-dione (33 mg, 35.73 μmol, 8.61% yield, 93% purity) as a yellow solid. MS(M+H)⁺=859.0

¹H NMR (400 MHz, DMSO-d₆) δ=11.09 (br s, 1H), 8.20 (s, 1H), 7.91-7.80 (m, 2H), 7.73-7.68 (m, 1H), 7.35 (t, J=7.2 Hz, 2H), 6.69 (d, J=7.9 Hz, 1H), 5.09 (br dd, J=5.4, 13.0 Hz, 1H), 4.74-4.63 (m, 1H), 4.01 (br t, J=14.2 Hz, 2H), 3.84 (s, 3H), 3.32-3.28 (m, 7H), 2.93-2.81 (m, 1H), 2.71-2.63 (m, 3H), 2.62-2.53 (m, 5H), 2.28-2.23 (m, 2H), 2.06-1.98 (m, 1H), 1.94-1.50 (m, 13H), 1.36-1.22 (m, 2H).

### Example 104. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(3-(((1-(2-(2,6-dioxopiperidin-3-yl)-1,3-dioxoisoindolin-5-yl)piperidin-4-yl)methyl)(methyl)amino)cyclobutyl)-3-methoxybenzamide (Compound 104)

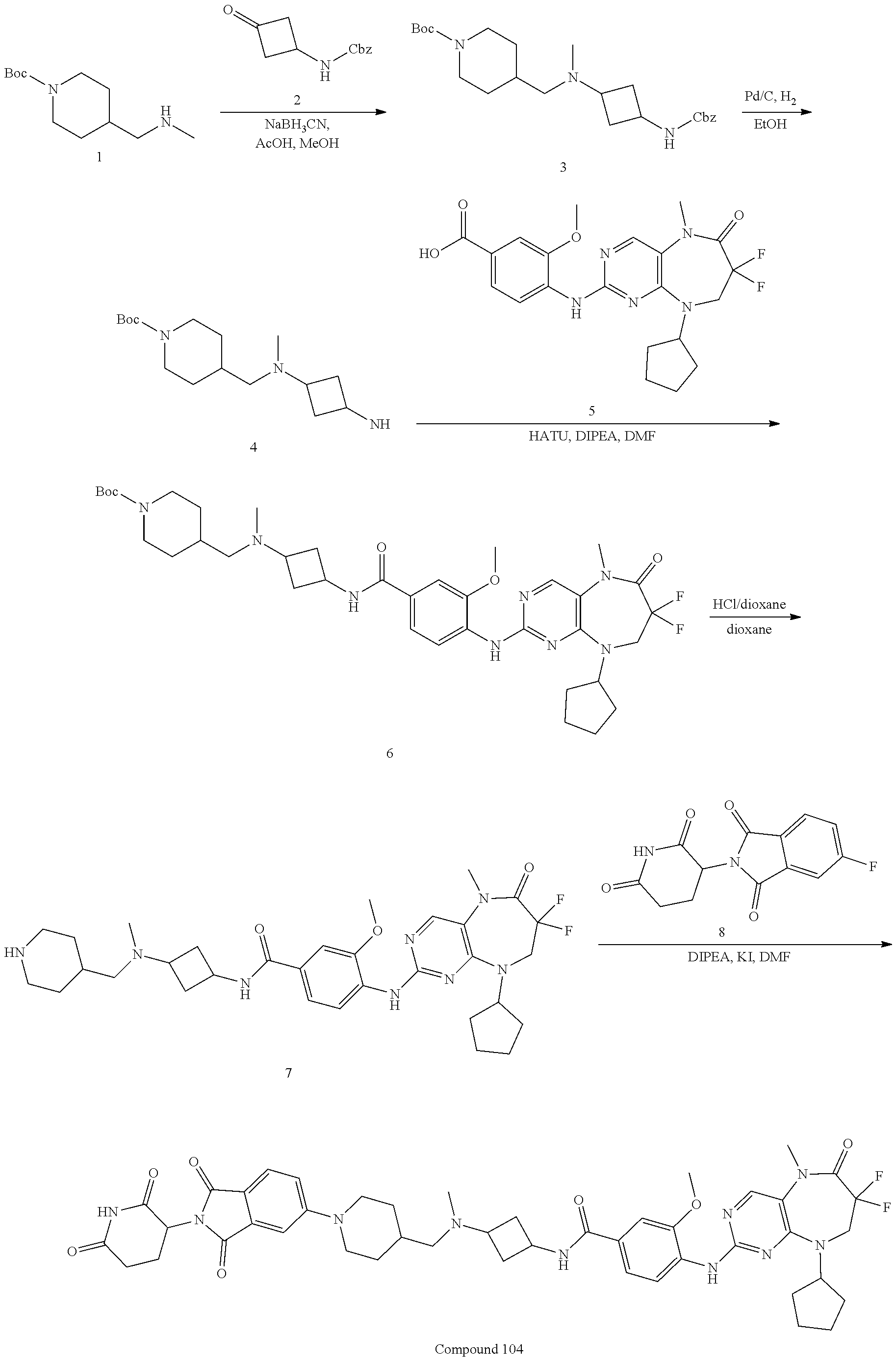

### Step 1. Synthesis of tert-butyl 4-(((3-(((benzyloxy)carbonyl)amino)cyclobutyl)(methyl)amino) methyl)piperidine-1-carboxylate (3)

To a solution of tert-butyl 4-((methylamino)methyl)piperidine-1-carboxylate (1 g, 4.38 mmol) and benzyl (3-oxocyclobutyl)carbamate (960.17 mg, 4.38 mmol) in MeOH (15 mL) was added NaBH₃CN (550.45 mg, 8.76 mmol) and AcOH (263.01 mg, 4.38 mmol, 250.48 μL). The mixture was stirred at 25° C. for 2 h. LC-MS showed benzyl (3-oxocyclobutyl)carbamate was consumed completely and one main peak with desired mass was detected. The reaction mixture was diluted with water 100 mL and extracted with EtOAc (30 mL×3). The combined organic layers were washed with brine 30 mL, dried over anhydrous sodium sulfate, filtered and concentrated under reduced pressure to give a residue. The residue was purified by column chromatography (SiO₂, Petroleum ether/EtOAc=1/0 to 0/1) to afford tert-butyl 4-(((3-(((benzyloxy)carbonyl)amino)cyclobutyl)(methyl)amino)methyl)piperidine-1-carboxylate (1.2 g, 2.03 mmol, 46.35% yield, 73% purity) as colorless oil. MS(M+H)⁺=432.2

### Step 2. Synthesis of tert-butyl 4-(((3-aminocyclobutyl)(methyl)amino)methyl)piperidine-1-carboxylate (4)

To a solution of tert-butyl 4-(((3-(((benzyloxy)carbonyl)amino)cyclobutyl)(methyl)amino)methyl)piperidine-1-carboxylate (1.2 g, 2.78 mmol) in EtOH (15 mL) was added Pd/C (10%, 150 mg) under N₂ atmosphere. The suspension was degassed and purged with H₂ for 3 times. The mixture was stirred under H₂ (15 Psi) at 25° C. for 16 h. TLC (Petroleum ether:EtOAc=1:1) indicated tert-butyl 4-(((3-(((benzyloxy)carbonyl)amino)cyclobutyl)(methyl)amino)methyl)piperidine-1-carboxylate was consumed completely and one new spot formed. The mixture was filtered and filter cake was washed with EtOAc (100 mL), the filtrate was concentrated in vacuo to afford tert-butyl 4-(((3-aminocyclobutyl)(methyl)amino)methyl)piperidine-1-carboxylate (700 mg, 2.35 mmol, 84.64% yield) as colorless oil. MS(M+H)⁺=298.4

### Step 3. Synthesis of tert-butyl 4-(((3-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)cyclobutyl)(methyl)amino)methyl)piperidine-1-carboxylate (6)

To a solution of tert-butyl 4-(((3-aminocyclobutyl)(methyl)amino)methyl)piperidine-1-carboxylate (400 mg, 1.34 mmol) and 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (601.72 mg, 1.34 mmol) in DMF (8 mL) was added HATU (767.02 mg, 2.02 mmol) and DIPEA (521.43 mg, 4.03 mmol, 702.74 μL). The mixture was stirred at 25° C. for 1 h. LC-MS showed 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid was consumed completely and one main peak with desired mass was detected. The reaction mixture was diluted with water 100 mL and extracted with EtOAc (30 mL×3). The combined organic layers were washed with brine 30 mL, dried over anhydrous sodium sulfate, filtered and concentrated under reduced pressure to give a residue. The residue was purified by column chromatography (SiO₂, Petroleum ether/EtOAc=1/0 to 0/1) to afford tert-butyl 4-(((3-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)cyclobutyl)(methyl)amino)methyl)piperidine-1-carboxylate (630 mg, 589.39 μmol, 43.83% yield, 68% purity) as a yellow solid. MS(M+H)⁺=727.4

### Step 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(3-(methyl(piperidin-4-ylmethyl)amino)cyclobutyl)benzamide (7)

A mixture of tert-butyl 4-(((3-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)cyclobutyl)(methyl)amino)methyl)piperidine-1-carboxylate (500 mg, 687.90 μmol) and HCl/dioxane (4 M, 3 mL) in DCM (3 mL) was stirred at 25° C. for 1 h. LC-MS showed tert-butyl 4-(((3-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)cyclobutyl)(methyl)amino)methyl)piperidine-1-carboxylate was consumed completely and one main peak with desired mass was detected. The reaction mixture was concentrated under reduced pressure to remove solvent to afford 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(3-(methyl(piperidin-4-ylmethyl)amino)cyclobutyl)benzamide (500 mg, crude, HCl salt) as a yellow solid. MS(M+H)⁺=627.4

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(3-(((1-(2-(2,6-dioxopiperidin-3-yl)-1,3-dioxoisoindolin-5-yl)piperidin-4-yl)methyl)(methyl)amino)cyclobutyl)-3-methoxybenzamide (Compound 104)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(3-(methyl(piperidin-4-ylmethyl)amino)cyclobutyl)benzamide (200 mg, 301.57 μmol, HCl salt) and 2-(2,6-dioxopiperidin-3-yl)-5-fluoroisoindoline-1,3-dione (83.30 mg, 301.57 μmol) in DMF (4 mL) were added KI (50.06 mg, 301.57 μmol) and DIPEA (116.93 mg, 904.71 μmol, 157.58 μL). The mixture was stirred at 80° C. for 2 h. LC-MS showed ˜9% of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(3-(methyl(piperidin-4-ylmethyl)amino)cyclobutyl)benzamide remained, several new peaks were shown on LC-MS and ˜18% of desired compound was detected. The reaction mixture was diluted with water 100 mL and extracted with EtOAc (30 mL×3). The combined organic layers were washed with brine 30 mL, dried over anhydrous sodium sulfate, filtered and concentrated under reduced pressure to give a residue. The residue was purified by prep-HPLC (column: Phenomenex luna C18 250×50 mm×15 μm; mobile phase: [water(FA)-ACN]; B %: 21%-51%, 10 min) to afford the product as Formic acid salt. Then the residue was purified by prep-HPLC (column: Waters Xbridge 150×25 mm×5 μm; mobile phase: [water(NH₄HCO₃)-ACN]; B %: 55%-85%, 7 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(3-(((1-(2-(2,6-dioxopiperidin-3-yl)-1,3-dioxoisoindolin-5-yl)piperidin-4-yl)methyl)(methyl)amino)cyclobutyl)-3-methoxybenzamide (29 mg, 32.52 μmol, 10.78% yield, 99% purity) as a yellow solid. MS(M+H)⁺=883.2

¹H NMR (400 MHz, DMSO-d₆) δ=11.07 (s, 1H), 8.44 (d, J=7.6 Hz, 1H), 8.31-8.23 (m, 2H), 7.96 (s, 1H), 7.65 (d, J=8.6 Hz, 1H), 7.55-7.46 (m, 2H), 7.31 (d, J=2.0 Hz, 1H), 7.23 (dd, J=2.1, 8.6 Hz, 1H), 5.06 (dd, J=5.4, 12.8 Hz, 1H), 4.83-4.72 (m, 1H), 4.17-4.00 (m, 5H), 3.94 (s, 3H), 3.31 (s, 3H), 3.04-2.92 (m, 2H), 2.90-2.83 (m, 1H), 2.63-2.54 (m, 2H), 2.46-2.36 (m, 3H), 2.08-1.91 (m, 8H), 1.87-1.69 (m, 7H), 1.66-1.56 (m, 4H), 1.19-1.07 (m, 2H).

### Example 105. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(3-(((1-(2-(2,6-dioxopiperidin-3-yl)-1,3-dioxoisoindolin-4-yl)piperidin-4-yl)methyl)(methyl)amino)cyclobutyl)-3-methoxybenzamide (Compound 105)

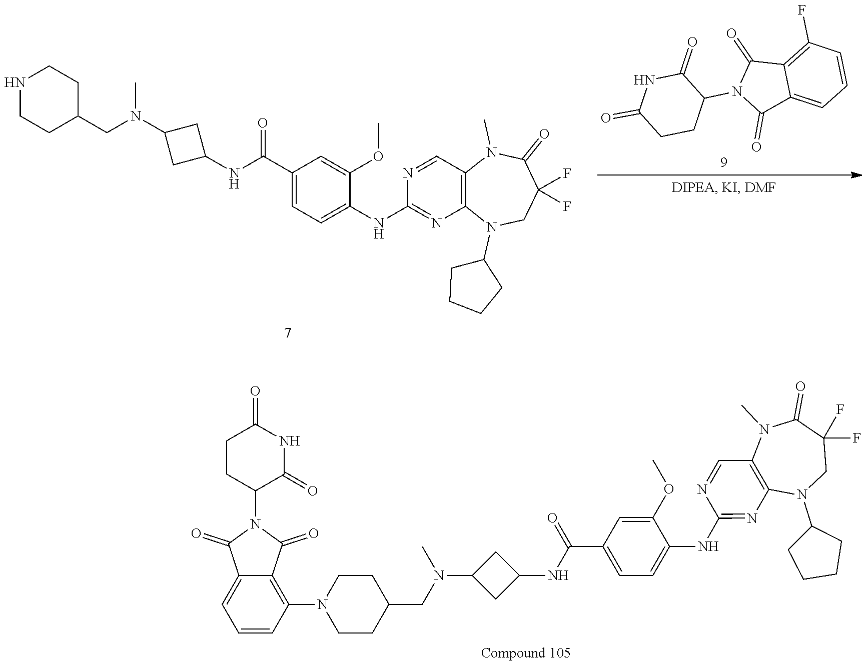 [SMILES: COc1cc(C(=O)NC2CC(N(C)CC3CCNCC3)C2)ccc1Nc1ncc2c(n1)N(C1CCCC1)CC(F)(F)C(=O)N2C ; O=C1CCC(N2C(=O)c3cccc(F)c3C2=O)C(=O)N1 ; "DIPEA, KI, DMF" ; COc1cc(C(=O)NC2CC(N(C)CC3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C]

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(3-(methyl(piperidin-4-ylmethyl)amino)cyclobutyl)benzamide (200 mg, 319.11 μmol) and 2-(2,6-dioxopiperidin-3-yl)-4-fluoroisoindoline-1,3-dione (88.15 mg, 319.11 μmol) in DMF (4 mL) were added KI (52.97 mg, 319.11 μmol) and DIPEA (123.73 mg, 957.34 μmol, 166.75 μL). The mixture was stirred at 90° C. for 3 h. LC-MS showed ˜55% of 2-(2,6-dioxopiperidin-3-yl)-4-fluoroisoindoline-1,3-dione remained, ˜10% m/z=517.1, ˜30% of desired compound was detected. The reaction mixture was diluted with water 100 mL and extracted with EtOAc (30 mL×3). The combined organic layers were washed with brine 30 mL, dried over anhydrous sodium sulfate, filtered and concentrated under reduced pressure to give a residue. The residue was purified by prep-HPLC (column: Phenomenex luna C18 250×50 mm×15 μm; mobile phase: [water (FA)-ACN]; B %: 21%-51%, 10 min) to afford the product as Formic acid salt. Then the residue was purified by prep-HPLC (column: Waters Xbridge 150×25 mm×5 μm; mobile phase: [water(NH₄HCO₃)-ACN]; B %: 58%-88%, 7 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(3-(((1-(2-(2,6-dioxopiperidin-3-yl)-1,3-dioxoisoindolin-4-yl)piperidin-4-yl)methyl)(methyl)amino)cyclobutyl)-3-methoxybenzamide (33.6 mg, 37.67 μmol, 11.81% yield, 99% purity) as a yellow solid. MS(M+H)⁺=883.3.

¹H NMR (400 MHz, DMSO-d₆) δ=11.08 (s, 1H), 8.45 (d, J=7.6 Hz, 1H), 8.31-8.24 (m, 2H), 7.96 (s, 1H), 7.68 (dd, J=7.3, 8.3 Hz, 1H), 7.55-7.48 (m, 2H), 7.37-7.29 (m, 2H), 5.08 (dd, J=5.4, 12.7 Hz, 1H), 4.83-4.72 (m, 1H), 4.17-4.01 (m, 3H), 3.95 (s, 3H), 3.75-3.63 (m, 2H), 3.33-3.29 (m, 3H), 2.93-2.83 (m, 3H), 2.63-2.57 (m, 2H), 2.47-2.38 (m, 3H), 2.14-2.00 (m, 7H), 1.97-1.90 (m, 2H), 1.89-1.81 (m, 4H), 1.74-1.68 (m, 2H), 1.65-1.57 (m, 4H), 1.38-1.23 (m, 2H).

### Example 106. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(3-((2-(1-(2-(2,6-dioxopiperidin-3-yl)-1,3-dioxoisoindolin-5-yl)piperidin-4-yl)ethyl)(methyl)amino)cyclobutyl)-3-methoxybenzamide (Compound 106)

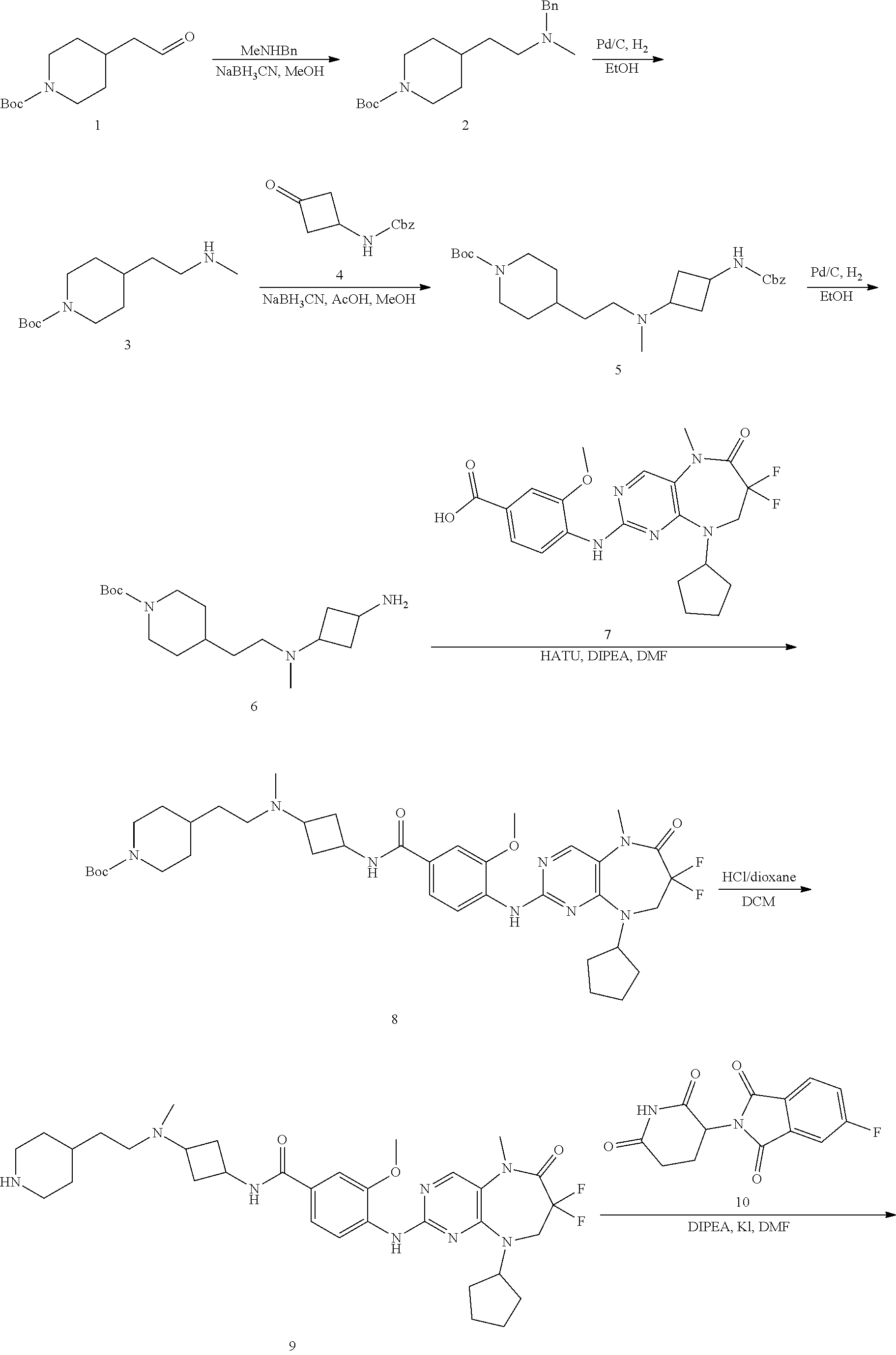

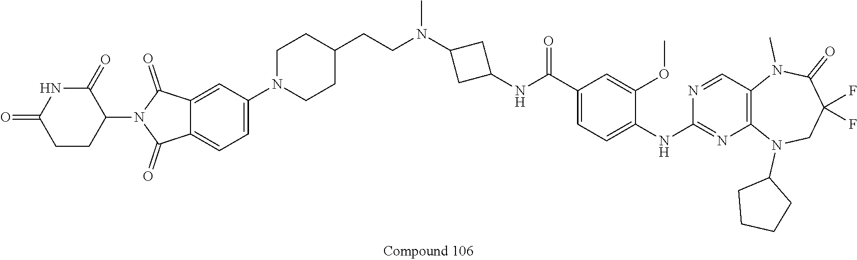 [SMILES: COc1cc(C(=O)NC2CC(N(C)CCC3CCN(c4ccc5c(c4)C(=O)N(C4CCC(=O)NC4=O)C5=O)CC3)C2)ccc1Nc1ncc2c(n1)N(C1CCCC1)CC(F)(F)C(=O)N2C]

### Step 1. Synthesis of tert-butyl 4-(2-(benzyl(methyl)amino)ethyl)piperidine-1-carboxylate (2)

To a solution of tert-butyl 4-(2-oxoethyl)piperidine-1-carboxylate (1.5 g, 6.60 mmol) and N-methyl-1-phenylmethanamine (799.69 mg, 6.60 mmol, 851.64 μL) in MeOH (6 mL) was added NaBH₃CN (1.24 g, 19.80 mmol) and AcOH (396.30 mg, 6.60 mmol, 377.43 μL). The mixture was stirred at 25° C. for 2 h. LCMS showed N-methyl-1-phenylmethanamine was consumed completely and one main peak with desired mass. The reaction mixture was diluted with water 200 mL and extracted with EtOAc (50 mL×3). The combined organic layers were washed with brine (50 mL), dried over anhydrous sodium sulfate, filtered and concentrated under reduced pressure. The residue was purified by reversed-phase HPLC (0.1% NH₃*H₂O) and the eluent was lyophilized to afford tert-butyl 4-(2-(benzyl(methyl)amino)ethyl)piperidine-1-carboxylate (2 g, 5.53 mmol, 83.86% yield, 92% purity) as colorless oil. MS(M+H)⁺=333.4

### Step 2. Synthesis of tert-butyl 4-(2-(methylamino)ethyl)piperidine-1-carboxylate (3)

To a solution of tert-butyl 4-(2-(methylamino)ethyl)piperidine-1-carboxylate (1.8 g, 5.41 mmol) in EtOH (20 mL) was added Pd/C (10%, 200 mg) under N₂ atmosphere. The suspension was degassed and purged with H₂ for 3 times. The mixture was stirred under H₂ (15 Psi) at 25° C. for 5 h. TLC (Petroleum ether/EtOAc=1/1) indicated tert-butyl 4-(2-(methylamino)ethyl)piperidine-1-carboxylate was consumed completely and one new spot formed. The mixture was filtered and filter cake was washed with EtOAc (200 mL), the filtrate was concentrated in vacuo to afford tert-butyl 4-(2-(methylamino)ethyl)piperidine-1-carboxylate (1.3 g, 5.36 mmol, 99.08% yield) as colorless oil. MS(M+H)⁺=243.4

### Step 3. Synthesis of tert-butyl 4-(2-((3-(((benzyloxy)carbonyl)amino)cyclobutyl)(methyl)amino)ethyl)piperidine-1-carboxylate (5)

To a solution of tert-butyl 4-(2-(methylamino)ethyl)piperidine-1-carboxylate (500 mg, 2.06 mmol) and benzyl (3-oxocyclobutyl)carbamate (452.30 mg, 2.06 mmol) in MeOH (7 mL) was added NaBH₃CN (324.12 mg, 5.16 mmol) and AcOH (123.89 mg, 2.06 mmol, 117.99 μL). The mixture was stirred at 25° C. for 2 h. LCMS showed benzyl (3-oxocyclobutyl)carbamate was consumed completely and a peak (71%) with desired mass. The reaction mixture was diluted with water (100 mL) and extracted with EtOAc (30 mL×3). The combined organic layers were washed with brine 30 mL, dried over anhydrous sodium sulfate, filtered and concentrated under reduced pressure. The residue was purified by reversed-phase HPLC (0.1% NH₃*H₂O) and the eluent was lyophilized to afford tert-butyl 4-(2-((3-(((benzyloxy)carbonyl)amino)cyclobutyl)(methyl)amino)ethyl)piperidine-1-carboxylate (800 mg, 1.80 mmol, 87.02% yield) as colorless oil. MS(M+H)⁺=446.8

### Step 4. Synthesis of tert-butyl 4-(2-((3-aminocyclobutyl)(methyl)amino)ethyl)piperidine-1-carboxylate (6)

To a solution of tert-butyl 4-(2-((3-(((benzyloxy)carbonyl)amino)cyclobutyl)(methyl)amino)ethyl)piperidine-1-carboxylate (800 mg, 1.80 mmol) in EtOH (10 mL) was added Pd/C (10%, 80 mg) under N₂ atmosphere. The suspension was degassed and purged with H₂ for 3 times. The mixture was stirred under H₂ (15 Psi) at 25° C. for 5 h. TLC (Petroleum ether/EtOAc=1/1) indicated tert-butyl 4-(2-((3-(((benzyloxy)carbonyl)amino)cyclobutyl)(methyl)amino)ethyl)piperidine-1-carboxylate was consumed completely and one new spot formed. The reaction mixture was filtered and filter cake was washed with EtOAc (100 mL), the filtrate was concentrated in vacuo to afford tert-butyl 4-(2-((3-aminocyclobutyl)(methyl)amino)ethyl)piperidine-1-carboxylate (500 mg, 1.61 mmol, 89.42% yield) as colorless oil. MS(M+H)⁺=312.5

### Step 5. Synthesis of tert-butyl 4-(2-((3-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)cyclobutyl) (methyl)amino)ethyl)piperidine-1-carboxylate (8)

To a solution of tert-butyl 4-(2-((3-aminocyclobutyl)(methyl)amino)ethyl)piperidine-1-carboxylate (400 mg, 1.28 mmol) and 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (574.63 mg, 1.28 mmol) in DMF (6 mL) was added HATU (732.48 mg, 1.93 mmol) and DIPEA (497.95 mg, 3.85 mmol, 671.09 μL). The mixture was stirred at 25° C. for 2 h. LCMS showed tert-butyl 4-(2-((3-aminocyclobutyl)(methyl)amino)ethyl)piperidine-1-carboxylate was consumed completely and one main peak with desired mass. The reaction mixture was diluted with water (100 mL) and extracted with EtOAc (30 mL×3). The combined organic layers were washed with brine (30 mL), dried over anhydrous sodium sulfate, filtered and concentrated under reduced pressure. The residue was purified by column chromatography (SiO₂, Petroleum ether/EtOAc=0/1 to 1/0) to afford tert-butyl 4-(2-((3-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)cyclobutyl)(methyl)amino)ethyl)piperidine-1-carboxylate (600 mg, 801.75 μmol, 62.43% yield, 99% purity) as a light yellow solid. MS(M+H)⁺=741.6

### Step 6. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(3-(methyl(2-(piperidin-4-yl)ethyl)amino)cyclobutyl)benzamide (9)

A mixture of tert-butyl 4-(2-((3-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)cyclobutyl)(methyl)amino)ethyl)piperidine-1-carboxylate (400 mg, 539.90 μmol), HCl/dioxane (4 M, 2.5 mL) in DCM (2.5 mL) was stirred at 25° C. for 1 h. LCMS showed starting material was consumed completely and one main peak with desired mass. The reaction mixture was concentrated under reduced pressure to afford 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(3-(methyl(2-(piperidin-4-yl)ethyl)amino)cyclobutyl)benzamide (400 mg, crude, HCl salt) as a yellow solid. MS(M+H)⁺=641.5

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(3-((2-(1-(2-(2,6-dioxopiperidin-3-yl)-1,3-dioxoisoindolin-5-yl)piperidin-4-yl)ethyl)(methyl)amino)cyclobutyl)-3-methoxybenzamide (Compound 106)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(3-(methyl(2-(piperidin-4-yl)ethyl)amino)cyclobutyl)benzamide (200 mg, 295.32 μmol, HCl salt) and 2-(2,6-dioxopiperidin-3-yl)-5-fluoroisoindoline-1,3-dione (81.57 mg, 295.32 μmol) in DMF (4 mL) was added KI (49.02 mg, 295.32 μmol) and DIPEA (114.51 mg, 885.97 μmol, 154.32 μL). The mixture was stirred at 90° C. for 5 h. LCMS showed 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(3-(methyl(2-(piperidin-4-yl)ethyl)amino)cyclobutyl)benzamide was consumed completely and a peak (19%) with desired mass. The reaction mixture was diluted with water (100 mL) and extracted with EtOAc (30 mL×3). The combined organic layers were washed with brine (30 mL), dried over anhydrous sodium sulfate, filtered and concentrated under reduced pressure. The residue was purified by prep-HPLC (condition: column: Waters Xbridge 150×25 mm×5 μm; mobile phase: [water(NH₄HCO₃)-ACN]; B %: 46%-76%, 9 min) and the eluent was lyophilized. The residue was purified by column chromatography (SiO₂, Petroleum ether/EtOAc=1/1 to 0/1) to afford 4-((9-cyclopentyl-7,7-difluoro-5-methyl-6-oxo-6,7,8,9-tetrahydro-5H-pyrimido[4,5-b][1,4]diazepin-2-yl)amino)-N-(3-((2-(1-(2-(2,6-dioxopiperidin-3-yl)-1,3-dioxoisoindolin-5-yl)piperidin-4-yl)ethyl)(methyl)amino)cyclobutyl)-3-methoxybenzamide (26.1 mg, 28.81 μmol, 9.75% yield, 99% purity) as a yellow solid. MS(M+H)⁺=897.0.

¹H NMR (400 MHz, DMSO-d₆) δ=11.07 (s, 1H), 8.46 (d, J=7.7 Hz, 1H), 8.31-8.23 (m, 2H), 8.00-7.94 (m, 1H), 7.65 (d, J=8.6 Hz, 1H), 7.56-7.46 (m, 2H), 7.29 (s, 1H), 7.22 (dd, J=2.0, 8.7 Hz, 1H), 5.06 (dd, J=5.3, 12.9 Hz, 1H), 4.83-4.69 (m, 1H), 4.21-4.10 (m, 1H), 4.08-4.00 (m, 4H), 3.93 (s, 3H), 3.33 (s, 3H), 2.98-2.84 (m, 3H), 2.62-2.55 (m, 3H), 2.48-2.37 (m, 2H), 2.31-2.22 (m, 2H), 2.02 (s, 3H), 1.99-1.84 (m, 5H), 1.79-1.68 (m, 4H), 1.67-1.56 (m, 5H), 1.38-1.32 (m, 2H), 1.27-1.09 (m, 2H).

### Example 107. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(3-((2-(1-(2-(2,6-dioxopiperidin-3-yl)-1,3-dioxoisoindolin-4-yl)piperidin-4-yl)ethyl)(methyl)amino)cyclobutyl)-3-methoxybenzamide (Compound 107)

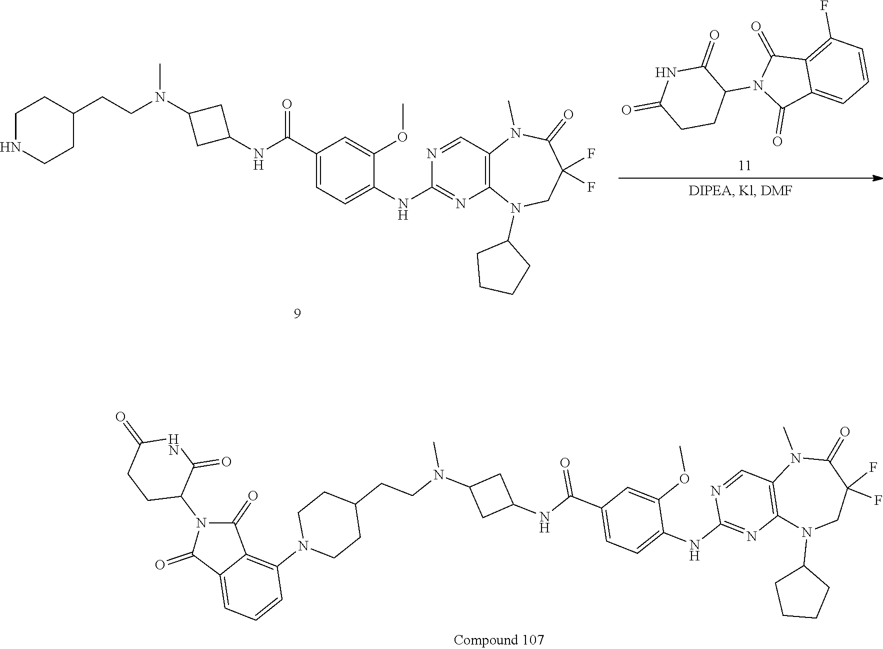 [SMILES: COc1cc(C(=O)NC2CC(N(C)CCC3CCNCC3)C2)ccc1Nc1ncc2c(n1)N(C1CCCC1)CC(F)(F)C(=O)N2C ; O=C1CCC(N2C(=O)c3cccc(F)c3C2=O)C(=O)N1 ; COc1cc(C(=O)NC2CC(N(C)CCC3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C]

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(3-(methyl(2-(piperidin-4-yl)ethyl)amino)cyclobutyl)benzamide (200 mg, 295.32 μmol, HCl salt) and 2-(2,6-dioxopiperidin-3-yl)-4-fluoroisoindoline-1,3-dione (81.57 mg, 295.32 μmol) in DMF (4 mL) was added KI (49.02 mg, 295.32 μmol) and DIPEA (114.51 mg, 885.97 μmol, 154.32 μL). The mixture was stirred at 90° C. for 5 h. LCMS showed 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(3-(methyl(2-(piperidin-4-yl)ethyl)amino)cyclobutyl)benzamide was consumed completely and a peak (46%) with desired mass. The reaction mixture was diluted with water (100 mL) and extracted with EtOAc (30 mL×3). The combined organic layers were washed with brine (30 mL), dried over anhydrous sodium sulfate, filtered and concentrated under reduced pressure. The residue was purified by column chromatography (SiO₂, Petroleum ether/EtOAc=1/1 to 0/1). The crude product was purified by prep-HPLC (condition column: Waters Xbridge 150×25 mm×5 μm; mobile phase: [water(NH₄HCO₃)-ACN]; B %: 47%-77%, 9 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(3-((2-(1-(2-(2,6-dioxopiperidin-3-yl)-1,3-dioxoisoindolin-4-yl)piperidin-4-yl)ethyl)(methyl)amino)cyclobutyl)-3-methoxybenzamide (51.9 mg, 57.28 μmol, 19.40% yield, 99% purity) as a yellow solid. MS(M+H)⁺=897.0.

¹H NMR (400 MHz, DMSO-d₆) δ=11.09 (s, 1H), 8.46 (d, J=7.7 Hz, 1H), 8.30-8.23 (m, 2H), 7.97 (s, 1H), 7.75-7.62 (m, 1H), 7.56-7.46 (m, 2H), 7.37-7.27 (m, 2H), 5.08 (dd, J=5.4, 12.8 Hz, 1H), 4.80-4.67 (m, 1H), 4.21-4.10 (m, 1H), 4.09-4.02 (m, 2H), 3.94 (s, 3H), 3.76-3.63 (m, 2H), 3.33 (s, 3H), 2.91-2.81 (m, 3H), 2.63-2.53 (m, 3H), 2.47-2.41 (m, 2H), 2.28-2.22 (m, 2H), 2.04 (s, 3H), 2.01-1.84 (m, 5H), 1.81-1.67 (m, 4H), 1.65-1.55 (m, 4H), 1.53-1.46 (m, 1H), 1.44-1.30 (m, 4H).

### Example 108. Synthesis of 4-(((R)-9-cyclopentyl-7-fluoro-5-methyl-6-oxo-7-vinyl-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (Compound 108)

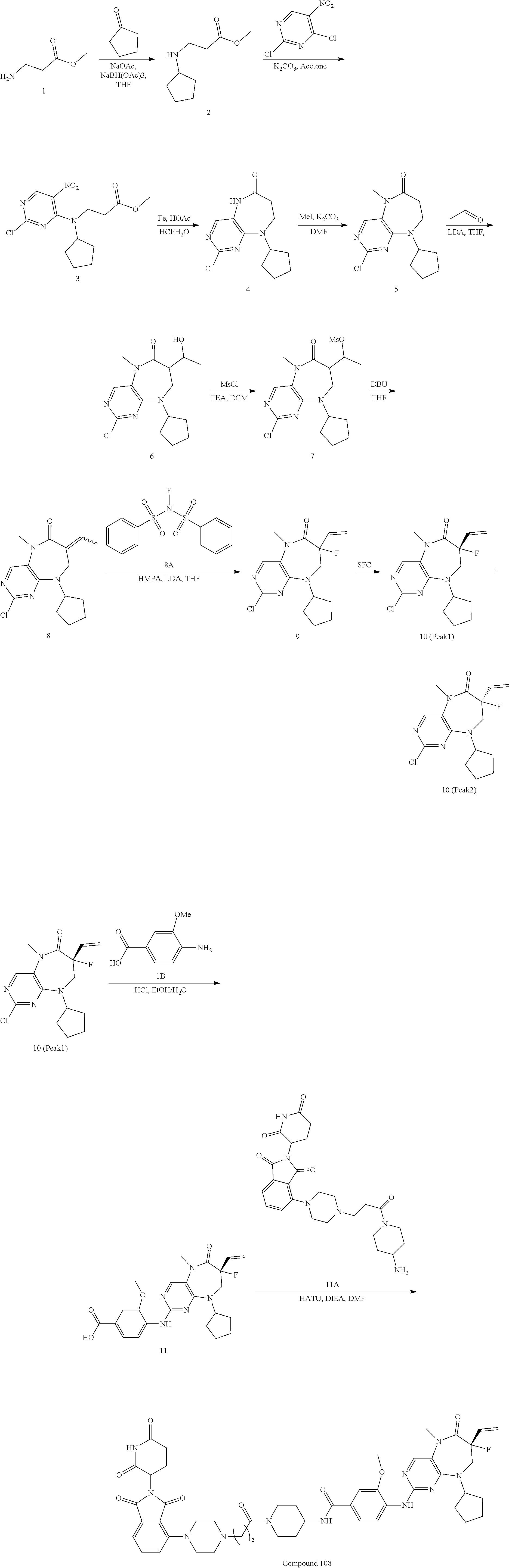

### Step 1. Synthesis of methyl 3-(cyclopentylamino)propanoate (2)

To the solution of ethyl 3-aminopropanoate (5 g, 32.55 mmol, HCl) and cyclopentanone (3.01 g, 35.81 mmol, 3.17 mL) in THF (100 mL) was added NaBH(OAc) 3 (10.64 g, 50.19 mmol) and NaOAc (2.95 g, 36.02 mmol) and the resulting mixture was stirred at 20° C. for 5 h. TLC (EtOAc) showed that starting material was consumed and new spots were formed. The mixture was poured into water (200 mL) and extracted with EtOAc (100 mL×3), the combined organic layer was washed with brine (200 mL×2), dried over Na₂SO₄, filtered and concentrated to afford methyl 3-(cyclopentylamino)propanoate (5.5 g, crude) as a yellow oil. MS(M+H)⁺=172.2

### Step 2. Synthesis of methyl 3-((2-chloro-5-nitropyrimidin-4-yl)(cyclopentyl)amino)propanoate (3)

To the solution of 2,4-dichloro-5-nitro-pyrimidine (6.23 g, 32.12 mmol) and methyl 3-(cyclopentylamino)propanoate (5.5 g, 32.12 mmol) in acetone (100 mL) was added K₂CO₃ (8.88 g, 64.24 mmol) and the resulting mixture was stirred at 20° C. for 12 h. TLC (Petroleum ether/EtOAc=5/1) showed that starting material was consumed and new spots were formed; LCMS showed that 74% desired mass was detected. The mixture was filtered and the filtrate was concentrated, the residue was purified by silica gel column (Petroleum ether/EtOAc=10/1-5/1) to afford methyl 3-((2-chloro-5-nitropyrimidin-4-yl)(cyclopentyl)amino)propanoate (5.5 g, 15.56 mmol, 48.44% yield, 93% purity) as a yellow solid. MS(M+H)⁺=329.1

### Step 3. Synthesis of 2-chloro-9-cyclopentyl-5,7,8,9-tetrahydro-6H-pyrimido[4,5-b][1,4]diazepin-6-one (4)

To the solution of methyl 3-((2-chloro-5-nitropyrimidin-4-yl)(cyclopentyl)amino)propanoate (4.5 g, 13.69 mmol) in AcOH (100 mL) and H₂O (100 mL) was added Fe (4.59 g, 82.13 mmol) and HCl (12 M, 3.6 mL) the resulting mixture stirred at 60° C. for 12 h. LCMS showed that starting material was consumed and 83% desired mass was detected. The mixture was filtered and concentrated, the residue was diluted with NaHCO₃ (sat, aq. 100 mL) and the resulting mixture was extracted with EtOAc (100 mL×3), the combined organic layer was washed with NaHCO₃ (200 mL×2), dried over Na₂SO₄, filtered and concentrated. The residue was triturated with (50 mL, Petroleum ether/EtOAc=5/1) and filtered and collected filter cake to afford 2-chloro-9-cyclopentyl-5,7,8,9-tetrahydro-6H-pyrimido[4,5-b][1,4]diazepin-6-one (3.2 g, 11.52 mmol, 84.14% yield, 96% purity) as a brown solid. MS(M+H)⁺=467.1

### Step 4. Synthesis of 2-chloro-9-cyclopentyl-5-methyl-5,7,8,9-tetrahydro-6H-pyrimido[4,5-b][1,4]diazepin-6-one (5)

To a mixture of 2-chloro-9-cyclopentyl-5,7,8,9-tetrahydro-6H-pyrimido[4,5-b][1,4]diazepin-6-one (2.7 g, 10.12 mmol) in DMF (50 mL) was added MeI (1.58 g, 11.14 mmol, 693.20 μL) and K₂CO₃ (2.80 g, 20.25 mmol) and the resulting mixture was stirred at 20° C. for 12 h. LCMS showed that starting material was consumed and 86% desired mass was detected. The mixture was poured into water (300 mL) and extracted with EtOAc (100 mL×3), the combined organic layer was washed with brine (300 mL), dried over Na₂SO₄, filtered and concentrated. The residue was purified by flash silica gel chromatography (50 g SepaFlash® Silica Flash Column, Eluent of 0˜50% EtOAc/Petroleum ether gradient @80 mL/min) to afford 2-chloro-9-cyclopentyl-5-methyl-5,7,8,9-tetrahydro-6H-pyrimido[4,5-b][1,4]diazepin-6-one (2 g, 7.05 mmol, 69.67% yield, 99% purity) as a yellow solid. MS(M+H)⁺=281.1

### Step 5. Synthesis of 2-chloro-9-cyclopentyl-7-(1-hydroxyethyl)-5-methyl-5,7,8,9-tetrahydro-6H-pyrimido[4,5-b][1,4]diazepin-6-one (6)

To the solution of 2-chloro-9-cyclopentyl-5-methyl-5,7,8,9-tetrahydro-6H-pyrimido[4,5-b][1,4]diazepin-6-one (1.5 g, 5.34 mmol) in THF (30 mL) was added LDA (2 M, 3.21 mL) at −78° C., the mixture was stirred at −78° C. for 30 min, acetaldehyde (941.45 mg, 21.37 mmol, 1.20 mL) was added and the resulting mixture was stirred at −78° C. for 2 h and stirred at 20° C. for another 12 h. LCMS showed that starting material was consumed and 83% desired mass was detected. The reaction was quenched with ammonium chloride (sat. aq. 50 mL), the mixture was extracted with EtOAc (2×50 mL) and the combined organic extracts were washed with brine (100 mL), dried over Na₂SO₄, filtered and concentrated in vacuo. The residue was purified by flash silica gel chromatography (20 g SepaFlash® Silica Flash Column, Eluent of 0˜50% EtOAc/Petroleum ether gradient @80 mL/min) to afford 2-chloro-9-cyclopentyl-7-(1-hydroxyethyl)-5-methyl-5,7,8,9-tetrahydro-6H-pyrimido[4,5-b][1,4]diazepin-6-one (2.0 g, crude) as a yellow oil. MS(M+H)⁺=452.2

### Step 6. Synthesis of 1-(2-chloro-9-cyclopentyl-5-methyl-6-oxo-6,7,8,9-tetrahydro-5H-pyrimido[4,5-b][1,4]diazepin-7-yl)ethyl methanesulfonate (7)

To a solution of 2-chloro-9-cyclopentyl-7-(1-hydroxyethyl)-5-methyl-5,7,8,9-tetrahydro-6H-pyrimido[4,5-b][1,4]diazepin-6-one (1.5 g, 4.62 mmol) and TEA (934.61 mg, 9.24 mmol, 1.29 mL) in DCM (30 mL) was added MsCl (793 mg, 6.92 mmol, 535.81 μL) at 0° C. and the resulting mixture was stirred at 20° C. for 12 h. LCMS showed that starting material was consumed and 88% desired mass was detected, the reaction mixture was diluted with ice-water (100 mL) and extracted with DCM (50 mL×3), the organic layer was washed with brine (100 mL×3), dried over Na₂SO₄, filtered and concentrated to afford 1-(2-chloro-9-cyclopentyl-5-methyl-6-oxo-6,7,8,9-tetrahydro-5H-pyrimido[4,5-b][1,4]diazepin-7-yl)ethyl methanesulfonate (1.6 g, 3.97 mmol, 85.99% yield) as a yellow oil. MS(M+H)⁺=403.1

### Step 7. Synthesis of 2-chloro-9-cyclopentyl-7-ethylidene-5-methyl-5,7,8,9-tetrahydro-6H-pyrimido[4,5-b][1,4]diazepin-6-one (8)

To the solution of 1-(2-chloro-9-cyclopentyl-5-methyl-6-oxo-6,7,8,9-tetrahydro-5H-pyrimido[4,5-b][1,4]diazepin-7-yl)ethyl methanesulfonate (1.6 g, 3.97 mmol) in THF (50 mL) was added DBU (1.81 g, 11.91 mmol, 1.80 mL) and the resulting mixture was stirred at 20° C. for 12 h. LCMS showed that starting material was consumed and 36% desired mass was detected. The reaction mixture was slowly added to ice cold water (20 mL) and extracted with EtOAc (2×20 mL), the combined organic layer was dried over Na₂SO₄, filtered and concentrated. The residue was purified by flash silica gel chromatography (20 g SepaFlash® Silica Flash Column, Eluent of 0˜50% EtOAc/Petroleum ether gradient @80 mL/min) to afford 2-chloro-9-cyclopentyl-7-ethylidene-5-methyl-5,7,8,9-tetrahydro-6H-pyrimido[4,5-b][1,4]diazepin-6-one (600 mg, 1.96 mmol, 49.25% yield, 100% purity) as a yellow solid. MS(M+H)⁺=307.1.

### Step 8. Synthesis of rel-(R)-2-chloro-9-cyclopentyl-7-fluoro-5-methyl-7-vinyl-5,7,8,9-tetrahydro-6H-pyrimido[4,5-b][1,4]diazepin-6-one (10 (Peak1)) and rel-(S)-2-chloro-9-cyclopentyl-7-fluoro-5-methyl-7-vinyl-5,7,8,9-tetrahydro-6H-pyrimido[4,5-b][1,4]diazepin-6-one (10 (Peak2))

To the solution of 2-chloro-9-cyclopentyl-7-ethylidene-5-methyl-5,7,8,9-tetrahydro-6H-pyrimido[4,5-b][1,4]diazepin-6-one (0.3 g, 977.87 μmol) in THF (10 mL) was LDA (2 M, 977.87 μL) and HMPA (350.47 mg, 1.96 mmol, 343.60 μL) at −78° C. and the resulting mixture was stirred at −78° C. for 0.5 h, then, N-(benzenesulfonyl)-N-fluoro-benzenesulfonamide (616.72 mg, 1.96 mmol) was added and the resulting mixture was stirred at −78° C. for 0.5 h and the elevated the temperature to 0° C. for 0.5 h. LCMS showed that starting material was consumed and desired mass was detected. The mixture was poured into water (50 mL) and extracted with EtOAc (30 mL×3), the combined organic layer was washed with brine (100 mL), dried over Na₂SO₄, filtered and concentrated. The residue was purified by flash silica gel chromatography (5 g SepaFlash® Silica Flash Column, Eluent of 0˜30% EtOAc/Petroleum ether gradient @50 mL/min) to afford 270 mg of mesomer. The mesomer was separated with SFC (column: DAICEL CHIRALPAK AY-H (250 mm*30 mm, 10 um); mobile phase: [0.1% NH₃H₂O ETOH]; B %: 50%-50%, 3.5; 45 min) to afford rel-(R)-2-chloro-9-cyclopentyl-7-fluoro-5-methyl-7-vinyl-5,7,8,9-tetrahydro-6H-pyrimido[4,5-b][1,4]diazepin-6-one (10 (Peak1)) (120 mg, 362.09 μmol, 18.51% yield, 98% purity) and rel-(S)-2-chloro-9-cyclopentyl-7-fluoro-5-methyl-7-vinyl-5,7,8,9-tetrahydro-6H-pyrimido[4,5-b][1,4]diazepin-6-one (10 (Peak2)) (100 mg, 301.74 μmol, 15.43% yield, 98% purity) as a yellow solid. MS(M+H)⁺=325.1.

### Step 9. Synthesis of (R)-4-((9-cyclopentyl-7-fluoro-5-methyl-6-oxo-7-vinyl-6,7,8,9-tetrahydro-5H-pyrimido[4,5-b][1,4]diazepin-2-yl)amino)-3-methoxybenzoic acid (11)

To the solution of rel-(R)-2-chloro-9-cyclopentyl-7-fluoro-5-methyl-7-vinyl-5,7,8,9-tetrahydro-6H-pyrimido[4,5-b][1,4]diazepin-6-one (10 (Peak1)) (100 mg, 307.90 μmol) and 4-amino-3-methoxybenzoic acid (77.20 mg, 461.85 μmol) in EtOH (1 mL) and H₂O (3 mL) was added HCl (12 M, 56.45 μL) and the resulting mixture was stirred at 100° C. for 12 h. LCMS showed that starting material was consumed and 60% desired mass was detected. The reaction mixture was concentrated, the residue was purified by prep-HPLC (column: 3_Phenomenex Luna C18 75*30 mm*3 um; mobile phase: [water (HCl)-ACN]; B %: 26%-46%, 7 min) and the eluent was lyophilized to (R)-4-((9-cyclopentyl-7-fluoro-5-methyl-6-oxo-7-vinyl-6,7,8,9-tetrahydro-5H-pyrimido [4,5-b][1,4]diazepin-2-yl)amino)-3-methoxybenzoic acid (32 mg, 70.26 μmol, 22.82% yield) as a brown solid. MS(M+H)⁺=456.1.

### Step 10. Synthesis of 4-(((R)-9-cyclopentyl-7-fluoro-5-methyl-6-oxo-7-vinyl-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (Compound 108)

To the solution of (R)-4-((9-cyclopentyl-7-fluoro-5-methyl-6-oxo-7-vinyl-6,7,8,9-tetrahydro-5H-pyrimido[4,5-b][1,4]diazepin-2-yl)amino)-3-methoxybenzoic acid (30 mg, 65.86 μmol) and 4-(4-(3-(4-aminopiperidin-1-yl)-3-oxopropyl)piperazin-1-yl)-2-(2,6-dioxopiperidin-3-yl)isoindoline-1,3-dione (35.11 mg, 65.86 μmol HCl) in DMF (1 mL) was added HATU (37.57 mg, 98.79 μmol) and DIPEA (25.54 mg, 197.58 μmol, 34.42 μL) and the resulting mixture was stirred at 20° C. for 1 h. LCMS showed that starting material was consumed and desired mass was detected, the mixture was poured into water (20 mL) and extracted with EtOAc (20 mL×3), the combined organic layer was washed with brine (50 mL), dried over Na₂SO₄, filtered and concentrated, the resulting mixture was purified by prep-HPLC (column: Waters Xbridge 150*25 mm*5 um; mobile phase: [water(NH₄HCO₃)-ACN]; B %: 37%-67%, 9 min) to afford 4-(((R)-9-cyclopentyl-7-fluoro-5-methyl-6-oxo-7-vinyl-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (25 mg, 26.50 μmol, 40.24% yield, 99% purity) as a yellow solid. MS(M+H)⁺=934.4.

¹H NMR (400 MHz, CDCl₃) δ=8.49 (d, J=8.5 Hz, 1H), 8.15 (br s, 1H), 7.96 (s, 1H), 7.71 (s, 1H), 7.60 (dd, J=3, 8.3 Hz, 1H), 7.45-7.39 (m, 2H), 7.24 (dd, J=0.6, 8.5 Hz, 1H), 7.18 (d, J=8.4 Hz, 1H), 6.10-5.96 (m, 2H), 5.50 (dd, J=2.1, 17.3 Hz, 1H), 5.37 (d, J=11.0 Hz, 1H), 4.96 (dd, J=5.3, 12.2 Hz, 2H), 4.65 (d, J=13.8 Hz, 1H), 4.31-4.17 (m, 1H), 3.99 (s, 3H), 3.95-3.90 (m, 1H), 3.84-3.78 (m, 1H), 3.78-3.68 (m, 1H), 3.42-3.36 (m, 5H), 3.27-3.22 (m, 1H), 2.93-2.79 (m, 5H), 2.78-2.71 (m, 5H), 2.65-2.59 (m, 2H), 2.23-2.19 (m, 1H), 2.15-2.02 (m, 4H), 1.81-1.66 (m, 5H), 1.59-1.40 (m, 5H).

### Example 109. Synthesis of 4-(((S)-9-cyclopentyl-7-fluoro-5-methyl-6-oxo-7-vinyl-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (Compound 109)

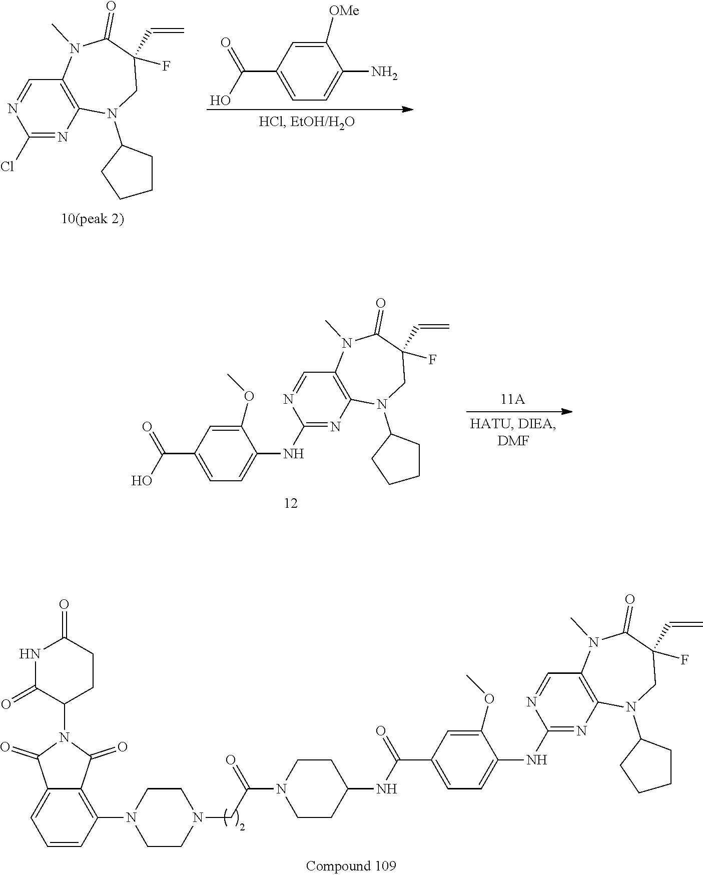

### Step 1. Synthesis of (S)-4-((9-cyclopentyl-7-fluoro-5-methyl-6-oxo-7-vinyl-6,7,8,9-tetrahydro-5H-pyrimido[4,5-b][1,4]diazepin-2-yl)amino)-3-methoxybenzoic acid (12)

To the solution of rel-(S)-2-chloro-9-cyclopentyl-7-fluoro-5-methyl-7-vinyl-5,7,8,9-tetrahydro-6H-pyrimido[4,5-b][1,4]diazepin-6-one (10 (Peak2)) (95 mg, 292.51 μmol,) and 4-amino-3-methoxybenzoic acid (73.34 mg, 438.76 μmol) in EtOH (1 mL) and H₂O (3 mL) was added HCl (12 M, 53.63 μL) and the resulting mixture was stirred at 100° C. for 12 h. LCMS showed that starting material was consumed and 66% desired mass was detected, the reaction mixture was concentrated, the residue was purified by prep-HPLC (column: 3_Phenomenex Luna C18 75*30 mm*3 um; mobile phase: [water (HCl)-ACN]; B %: 27%-47%, 7 min) and the eluent was lyophilized to afford (S)-4-((9-cyclopentyl-7-fluoro-5-methyl-6-oxo-7-vinyl-6,7,8,9-tetrahydro-5H-pyrimido[4,5-b][1,4]diazepin-2-yl)amino)-3-methoxybenzoic acid (35 mg, 76.84 μmol, 26.27% yield) as a brown solid. MS(M+H)⁺=456.2.

### Step 2. Synthesis of 4-(((S)-9-cyclopentyl-7-fluoro-5-methyl-6-oxo-7-vinyl-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (Compound 109)

To the solution of (S)-4-((9-cyclopentyl-7-fluoro-5-methyl-6-oxo-7-vinyl-6,7,8,9-tetrahydro-5H-pyrimido[4,5-b][1,4]diazepin-2-yl)amino)-3-methoxybenzoic acid (35 mg, 76.84 μmol) and 4-(4-(3-(4-aminopiperidin-1-yl)-3-oxopropyl)piperazin-1-yl)-2-(2,6-dioxopiperidin-3-yl)isoindoline-1,3-dione (40.96 mg, 76.84 μmol, HCl) in DMF (1 mL) was added HATU (43.83 mg, 115.26 μmol) and DIPEA (29.79 mg, 230.52 μmol, 40.15 μL) and the resulting mixture was stirred at 20° C. for 1 h. LCMS showed that starting material was consumed desired mass was detected. The mixture was poured into water (20 mL) and extracted with EtOAc (20 mL×3), the combined organic layer was washed with brine (50 mL), dried over Na₂SO₄, filtered and concentrated. The resulting mixture was purified by prep-HPLC (column: Waters Xbridge 150*25 mm*5 um; mobile phase: [water(NH₄HCO₃)-ACN]; B %: 36%-66%, 9 min) to afford 4-(((S)-9-cyclopentyl-7-fluoro-5-methyl-6-oxo-7-vinyl-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (24 mg, 25.18 μmol, 32.77% yield, 98% purity) as a yellow solid. MS(M+H)⁺=934.4

¹H NMR (400 MHz, CDCl₃) δ=8.49 (d, J=8.4 Hz, 1H), 8.08 (br s, 1H), 7.97 (s, 1H), 7.71 (s, 1H), 7.61 (dd, J=7.2, 8.3 Hz, 1H), 7.44-7.40 (m, 2H), 7.24 (dd, J=1.9, 8.6 Hz, 1H), 7.18 (d, J=8.1 Hz, 1H), 6.10-5.95 (m, 2H), 5.51 (dd, J=2.1, 17.3 Hz, 1H), 5.37 (d, J=11.0 Hz, 1H), 5.00-4.92 (m, 2H), 4.65 (d, J=12.9 Hz, 1H), 4.31-4.17 (m, 1H), 3.99 (s, 3H), 3.95-3.91 (m, 1H), 3.84-3.78 (m, 1H), 3.78-3.67 (m, 1H), 3.42-3.37 (m, 5H), 3.27-3.22 (m, 1H), 2.95-2.80 (m, 5H), 2.79-2.71 (m, 5H), 2.67-2.63 (m, 2H), 2.23-2.19 (m, 1H), 2.15-2.01 (m, 4H), 1.80-1.65 (m, 5H), 1.57-1.40 (m, 5H).

### Example 110. Synthesis of 4-(4-(2-(1-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxyphenyl)piperidin-4-yl)ethyl)piperazin-1-yl)-2-(2,6-dioxopiperidin-3-yl)isoindoline-1,3-dione (Compound 110)

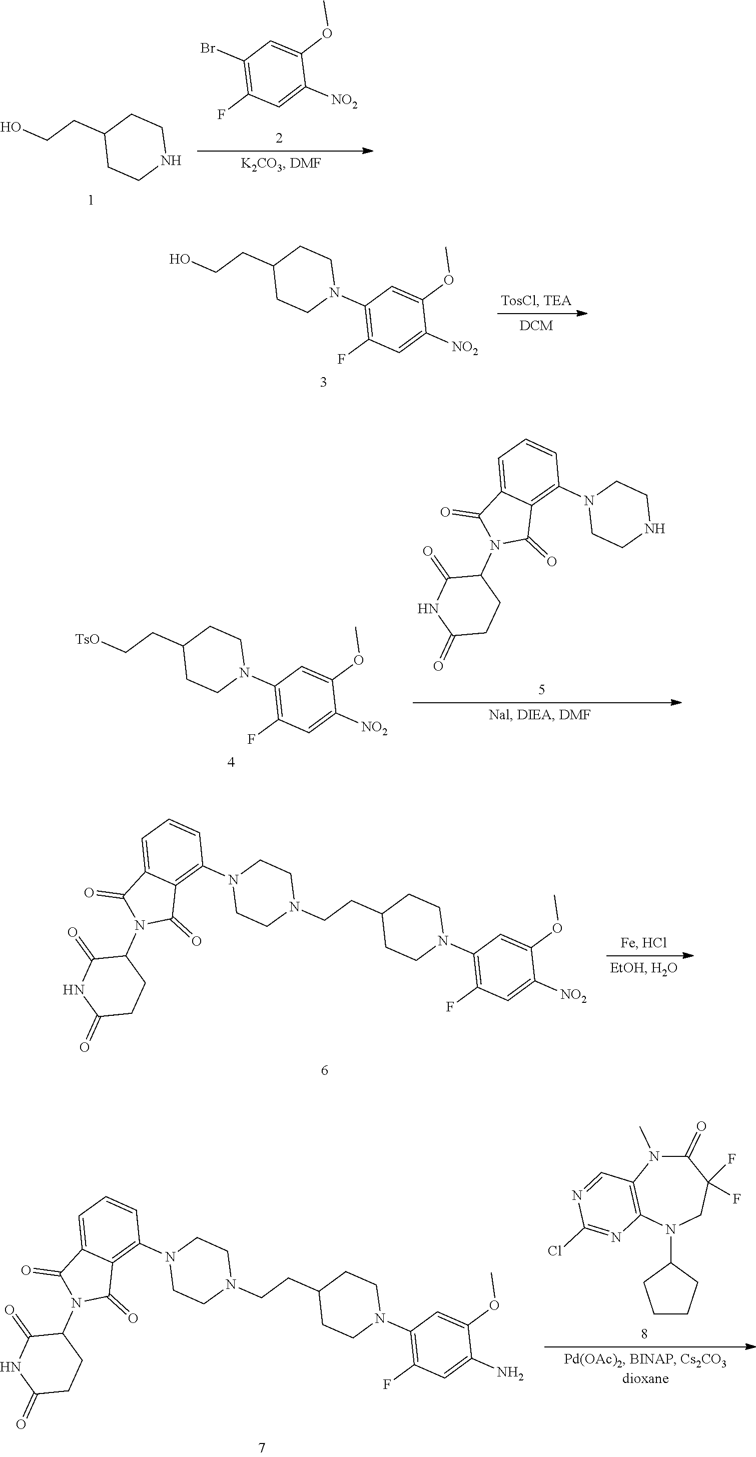

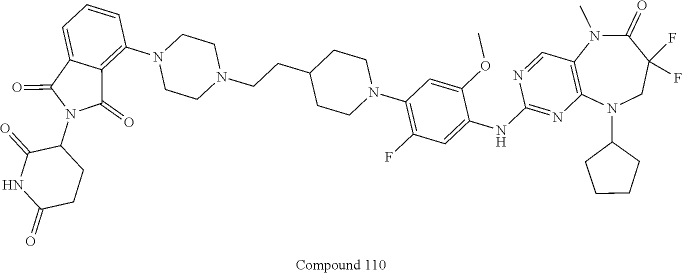 [SMILES: COc1cc(N2CCC(CCN3CCN(c4cccc5c4C(=O)N(C4CCC(=O)NC4=O)C5=O)CC3)CC2)c(F)cc1Nc1ncc2c(n1)N(C1CCCC1)CC(F)(F)C(=O)N2C]

### Step 1. Synthesis of 2-(1-(2-fluoro-5-methoxy-4-nitrophenyl)piperidin-4-yl)ethan-1-ol (3)

To a solution of 1-bromo-2-fluoro-5-methoxy-4-nitrobenzene (1.5 g, 6.00 mmol) and 2-(piperidin-4-yl)ethan-1-ol (1 g, 7.74 mmol) in DMF (15 mL) was added K₂CO₃ (1.66 g, 12.00 mmol) and the mixture was stirred at 80° C. for 14 h. LCMS showed a main peak (100%) with desired mass. The mixture was diluted with H₂O (10 mL) and extracted with EtOAc (10 mL×3). The combined organic layer was washed with brine (10 mL×3) and saturation citric acid (10 mL×3), dried over Na₂SO₄ and filtered. The filtrate was concentrated under reduced pressure to afford 2-(1-(2-fluoro-5-methoxy-4-nitrophenyl)piperidin-4-yl)ethan-1-ol (1.1 g, crude) as a yellow oil. MS(M+H)⁺=299.0

### Step 2. Synthesis of 2-(1-(2-fluoro-5-methoxy-4-nitrophenyl)piperidin-4-yl)ethyl 4-methylbenzenesulfonate (4)

To a solution of 2-(1-(2-fluoro-5-methoxy-4-nitrophenyl)piperidin-4-yl)ethan-1-ol (1.1 g, 3.69 mmol) in DCM (20 mL) were added TEA (1.12 g, 11.06 mmol, 1.54 mL) and TosCl (1.41 g, 7.37 mmol) and the mixture was stirred at 25° C. for 14 h. LCMS showed a peak (71%) with desired mass. The mixture was concentrated under reduced pressure. The residue was purified by flash silica gel chromatography (12 g SepaFlash® Silica Flash Column, Eluent of 30˜70% EtOAc/Petroleum ether gradient @50 mL/min) to afford 2-(1-(2-fluoro-5-methoxy-4-nitrophenyl)piperidin-4-yl)ethyl 4-methylbenzenesulfonate (1.4 g, 3.00 mmol, 81.39% yield, 97% purity) as a yellow solid. MS(M+H)⁺=453.2

### Step 3. Synthesis of 2-(2,6-dioxopiperidin-3-yl)-4-(4-(2-(1-(2-fluoro-5-methoxy-4-nitrophenyl) piperidin-4-yl)ethyl)piperazin-1-yl)isoindoline-1,3-dione (6)

To a solution of 2-(1-(2-fluoro-5-methoxy-4-nitrophenyl)piperidin-4-yl)ethyl 4-methylbenzenesulfonate (1.4 g, 3.09 mmol) and 2-(2,6-dioxopiperidin-3-yl)-4-(piperazin-1-yl)isoindoline-1,3-dione (780 mg, 2.06 mmol, HCl salt) in DMF (15 mL) were added NaI (15.46 mg, 103.13 μmol) and DIPEA (799.74 mg, 6.19 mmol, 1.08 mL) and the mixture was stirred at 50° C. for 50 h. LCMS showed a peak (46%) with desired mass. The mixture was diluted with H₂O (20 mL) and extracted with EtOAc (10 mL×3). The combined organic layer was washed with brine (10 mL×3), dried over Na₂SO₄ and filtered. The filtrate was concentrated under reduced pressure. The crude was diluted with EtOAc (10 mL) and stirred at 25° C. for 1 h. The mixture was filtered and the filter cake was washed with EtOAc (10 mL). The filter cake was collected and dried in vacuo to afford 2-(2,6-dioxopiperidin-3-yl)-4-(4-(2-(1-(2-fluoro-5-methoxy-4-nitrophenyl)piperidin-4-yl)ethyl)piperazin-1-yl)isoindoline-1,3-dione (590 mg, 871.77 μmol, 42.26% yield, 92% purity) as a yellow solid. MS(M+H)⁺=623.0

### Step 4. Synthesis of 4-(4-(2-(1-(4-amino-2-fluoro-5-methoxyphenyl)piperidin-4-yl)ethyl)piperazin-1-yl)-2-(2,6-dioxopiperidin-3-yl)isoindoline-1,3-dione (7)

To a solution of 2-(2,6-dioxopiperidin-3-yl)-4-(4-(2-(1-(2-fluoro-5-methoxy-4-nitrophenyl)piperidin-4-yl)ethyl)piperazin-1-yl)isoindoline-1,3-dione (0.59 g, 947.57 μmol) in EtOH (5 mL) and H₂O (5 mL) were Fe (317.50 mg, 5.69 mmol) and HCl (12 M, 470 μL) and the mixture was stirred at 80° C. for 1 h. LCMS showed a main peak (94%) with desired mass. The mixture was adjusted the pH=8 with saturated NaHCO₃ at 0° C. and diluted with DMF (20 mL) and H₂O (40 mL). The mixture was filtered and the filter cake was washed with DMF (50 mL). The filtrate was extracted with EtOAc (20 mL×3), the organic layer was washed with brine (10 mL×2), dried over Na₂SO₄, filtered and concentrated under reduced pressure to afford 4-(4-(2-(1-(4-amino-2-fluoro-5-methoxyphenyl)piperidin-4-yl)ethyl)piperazin-1-yl)-2-(2,6-dioxopiperidin-3-yl)isoindoline-1,3-dione (0.4 g, crude) as a yellow solid. MS(M+H)⁺=593.0

### Step 5. Synthesis of 4-(4-(2-(1-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxyphenyl)piperidin-4-yl)ethyl)piperazin-1-yl)-2-(2,6-dioxopiperidin-3-yl)isoindoline-1,3-dione (Compound 110)

To a solution of 2-chloro-9-cyclopentyl-7,7-difluoro-5-methyl-5,7,8,9-tetrahydro-6H-pyrimido[4,5-b][1,4]diazepin-6-one (118 mg, 372.55 μmol) and 4-(4-(2-(1-(4-amino-2-fluoro-5-methoxyphenyl)piperidin-4-yl)ethyl)piperazin-1-yl)-2-(2,6-dioxopiperidin-3-yl)isoindoline-1,3-dione (200 mg, 337.46 μmol) in dioxane (6 mL) were added Pd(OAc)₂ (7.58 mg, 33.75 μmol), Cs₂CO₃ (330.00 mg, 1.01 mmol) and BINAP (42.03 mg, 67.49 μmol) and the mixture was stirred at 100° C. for 28 h. LCMS showed a peak (41%) with desired mass. The mixture was diluted with THF (5 mL) and DCM (5 mL) and then filtered. The filter cake was washed with THF (10 mL) and DCM (10 mL). The filtrate was concentrated under reduced pressure. The residue was purified by flash silica gel chromatography (5 g SepaFlash® Silica Flash Column, Eluent of 60˜100% EtOAc/Petroleum ether to 5% MeOH/EtOAc gradient @50 mL/min) followed by prep-HPLC (column: Unisil 3-100 C18 Ultra 150*50 mm*3 um; mobile phase: [water(FA)-ACN]; B %: 15%-45%, 10 min) and the eluent was lyophilized to afford 4-(4-(2-(1-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxyphenyl)piperidin-4-yl)ethyl) piperazin-1-yl)-2-(2,6-dioxopiperidin-3-yl)isoindoline-1,3-dione (54.2 mg, 60.85 μmol, 18.03% yield, 98% purity, 0.25 FA) as a yellow solid. MS(M+H)⁺=873.0

¹H NMR (400 MHz, DMSO-d₆) δ=11.09 (s, 1H), 8.32 (s, 0.25H), 8.20 (s, 1H), 7.88 (d, J=14.7 Hz, 1H), 7.81 (s, 1H), 7.73-7.67 (m, 1H), 7.37-7.32 (m, 2H), 6.68 (d, J=8.3 Hz, 1H), 5.13-5.05 (m, 1H), 4.74-4.64 (m, 1H), 4.01 (br t, J=14.1 Hz, 2H), 3.84 (s, 3H), 3.37-3.32 (m, 7H), 2.93-2.82 (m, 1H), 2.66-2.64 (m, 1H), 2.63-2.59 (m, 2H), 2.58-2.55 (m, 5H), 2.43-2.38 (m, 2H), 2.07-1.98 (m, 1H), 1.94-1.85 (m, 2H), 1.82-1.75 (m, 2H), 1.72-1.24 (m, 13H).

### Example 111. Synthesis of 5-(4-((1-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxyphenyl)piperidin-4-yl)methyl)piperazin-1-yl)-2-(2,6-dioxopiperidin-3-yl)isoindoline-1,3-dione (Compound 111)

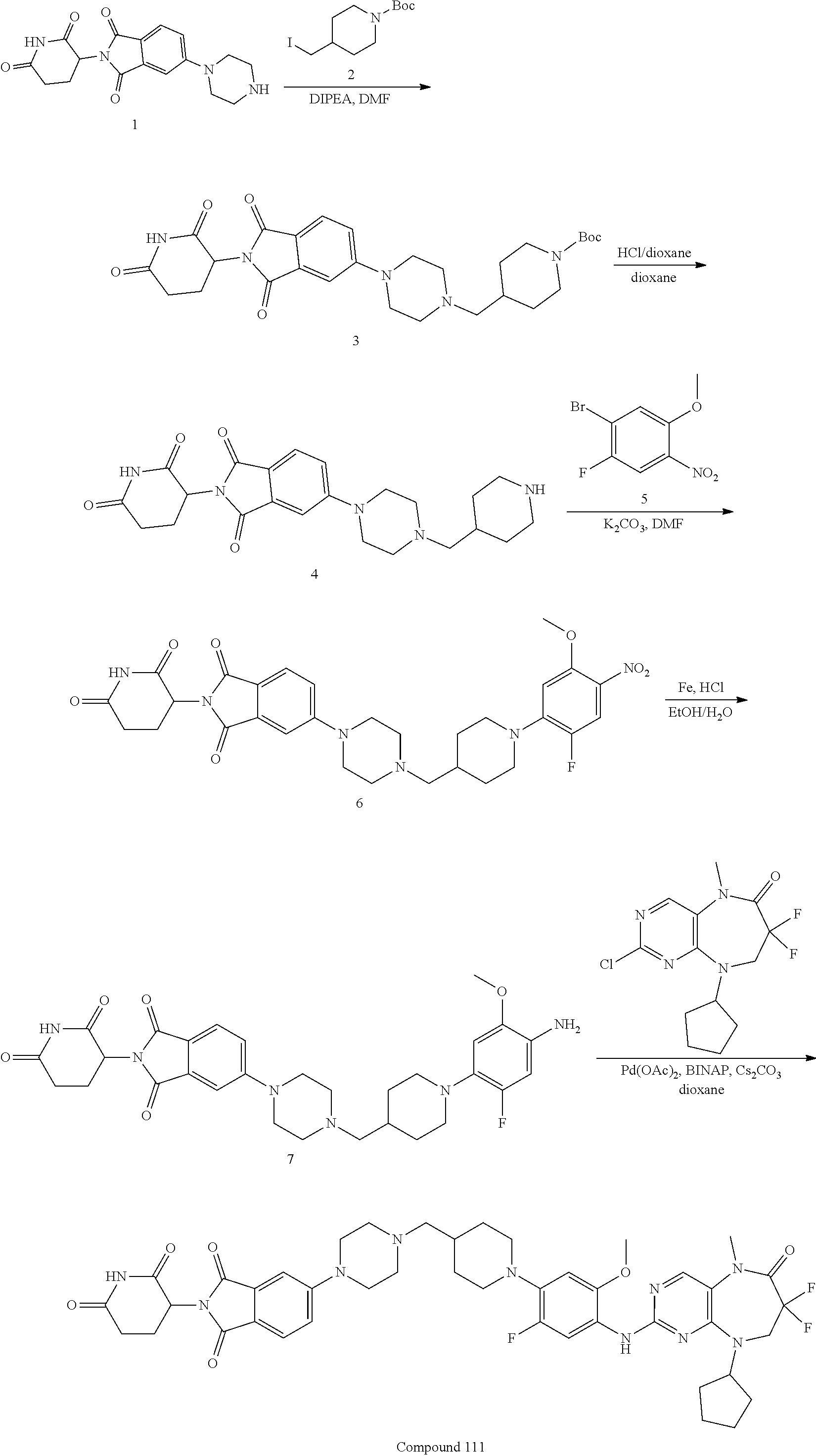

The compound 111 was synthesized by the method described in the scheme similar to the method described in Examples 89 and 103.

MS(M+H)⁺=859.4, ¹H NMR (400 MHz, DMSO-d₆) δ=11.08 (s, 1H), 8.20 (s, 1H), 7.88 (d, J=14.7 Hz, 1H), 7.81 (s, 1H), 7.68 (d, J=8.6 Hz, 1H), 7.36-7.33 (m, 1H), 7.28-7.23 (m, 1H), 6.69 (d, J=8.2 Hz, 1H), 5.07 (dd, J=5.4, 13.0 Hz, 1H), 4.74-4.62 (m, 1H), 4.01 (t, J=14.1 Hz, 2H), 3.84 (s, 3H), 3.48-3.42 (m, 4H), 3.31 (s, 3H), 2.94-2.82 (m, 1H), 2.73-2.64 (m, 2H), 2.57-2.51 (m, 6H), 2.27-2.22 (m, 2H), 2.05-1.97 (m, 1H), 1.95-1.79 (m, 4H), 1.73-1.49 (m, 8H), 1.36-1.21 (m, 3H).

### Example 112. Synthesis of 5-(4-(2-(1-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxyphenyl)piperidin-4-yl)ethyl)piperazin-1-yl)-2-(2,6-dioxopiperidin-3-yl)isoindoline-1,3-dione (Compound 112)

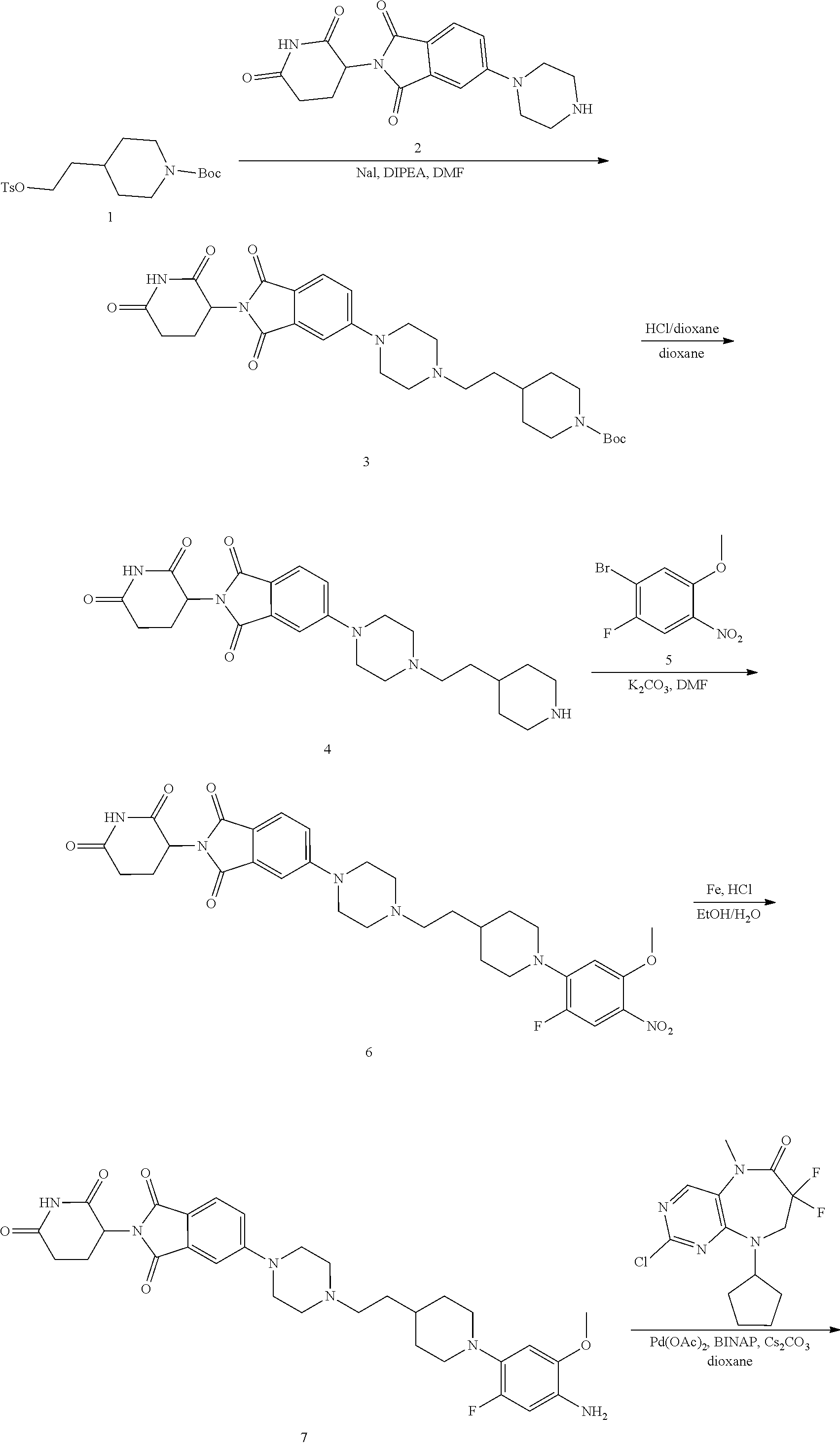

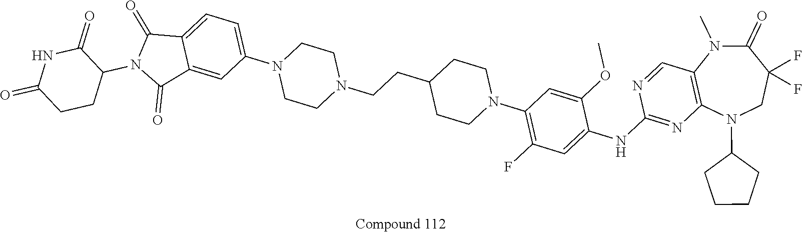 [SMILES: COc1cc(N2CCC(CCN3CCN(c4ccc5c(c4)C(=O)N(C4CCC(=O)NC4=O)C5=O)CC3)CC2)c(F)cc1Nc1ncc2c(n1)N(C1CCCC1)CC(F)(F)C(=O)N2C]

The compound 111 was synthesized by the method described in the scheme similar to the method described in Examples 89 and 103.

MS(M+H)⁺=873.0, ¹H NMR (400 MHz, CDCl₃) δ=8.20 (d, J=14.7 Hz, 1H), 8.07-8.04 (m, 1H), 8.01 (s, 1H), 7.71 (d, J=8.6 Hz, 1H), 7.47 (s, 1H), 7.31-7.29 (m, 1H), 7.10-7.06 (m, 1H), 6.57 (d, J=7.7 Hz, 1H), 4.95 (dd, J=5.2, 12.4 Hz, 1H), 4.88-4.79 (m, 1H), 3.94-3.83 (m, 5H), 3.49-3.44 (m, 3H), 3.42-3.36 (m, 4H), 2.94-2.61 (m, 9H), 2.55-2.46 (m, 2H), 2.18-2.04 (m, 3H), 1.87-1.73 (m, 7H), 1.61-1.44 (m, 8H).

### Example 113. Synthesis of 5-(4-(3-(1-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxyphenyl)piperidin-4-yl) propyl)piperazin-1-yl)-2-(2,6-dioxopiperidin-3-yl)isoindoline-1,3-dione (Compound 113)

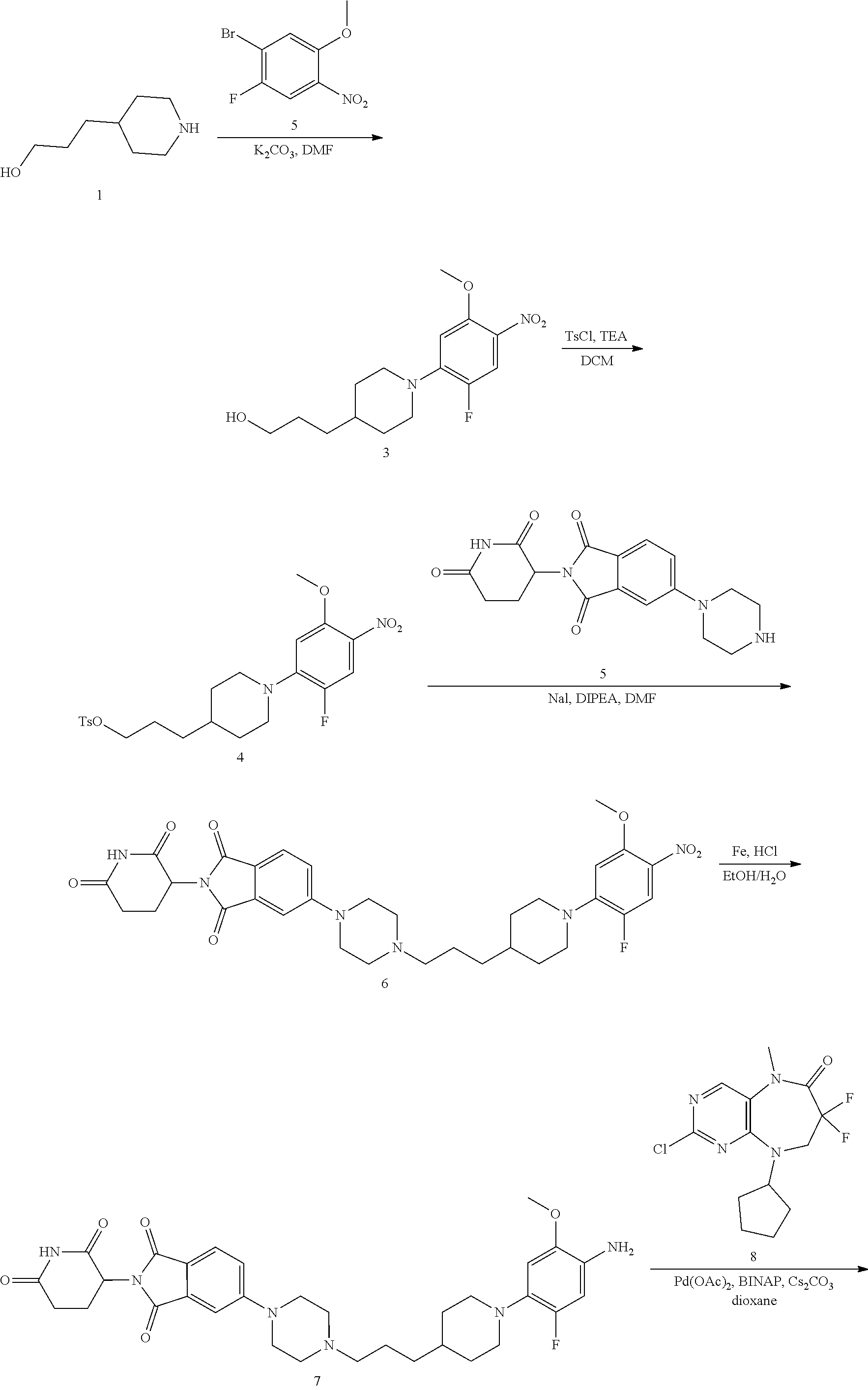

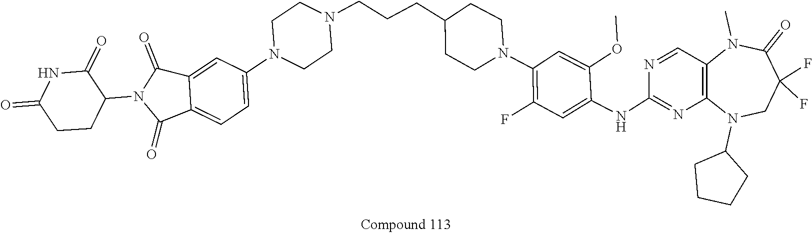 [SMILES: COc1cc(N2CCC(CCCN3CCN(c4ccc5c(c4)C(=O)N(C4CCC(=O)NC4=O)C5=O)CC3)CC2)c(F)cc1Nc1ncc2c(n1)N(C1CCCC1)CC(F)(F)C(=O)N2C]

The compound 113 was synthesized by the method described in the scheme similar to the method described in Example 110.

MS(M+H)⁺=887.3, ¹H NMR (400 MHz, CDCl₃) δ=8.34-8.13 (m, 2H), 8.00 (s, 1H), 7.71 (d, J=8.4 Hz, 1H), 7.51 (s, 1H), 7.31 (d, J=2.0 Hz, 1H), 7.11-7.03 (m, 1H), 6.57 (d, J=7.8 Hz, 1H), 4.99-4.91 (m, 1H), 4.90-4.79 (m, 1H), 3.95-3.82 (m, 5H), 3.53-3.46 (m, 4H), 3.42-3.36 (m, 5H), 2.93-2.64 (m, 9H), 2.54-2.45 (m, 2H), 2.16-2.06 (m, 4H), 1.86-1.78 (m, 2H), 1.76-1.72 (m, 3H), 1.67-1.55 (m, 4H), 1.49-1.41 (m, 2H), 1.40-1.30 (m, 3H).

### Example 114. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(4-(2-(1-(2-(2,6-dioxopiperidin-3-yl)-1,3-dioxoisoindolin-4-yl)piperidin-4-yl)ethyl)piperazin-1-yl)cyclohexyl)-2-fluoro-5-methoxybenzamide (Compound 114)

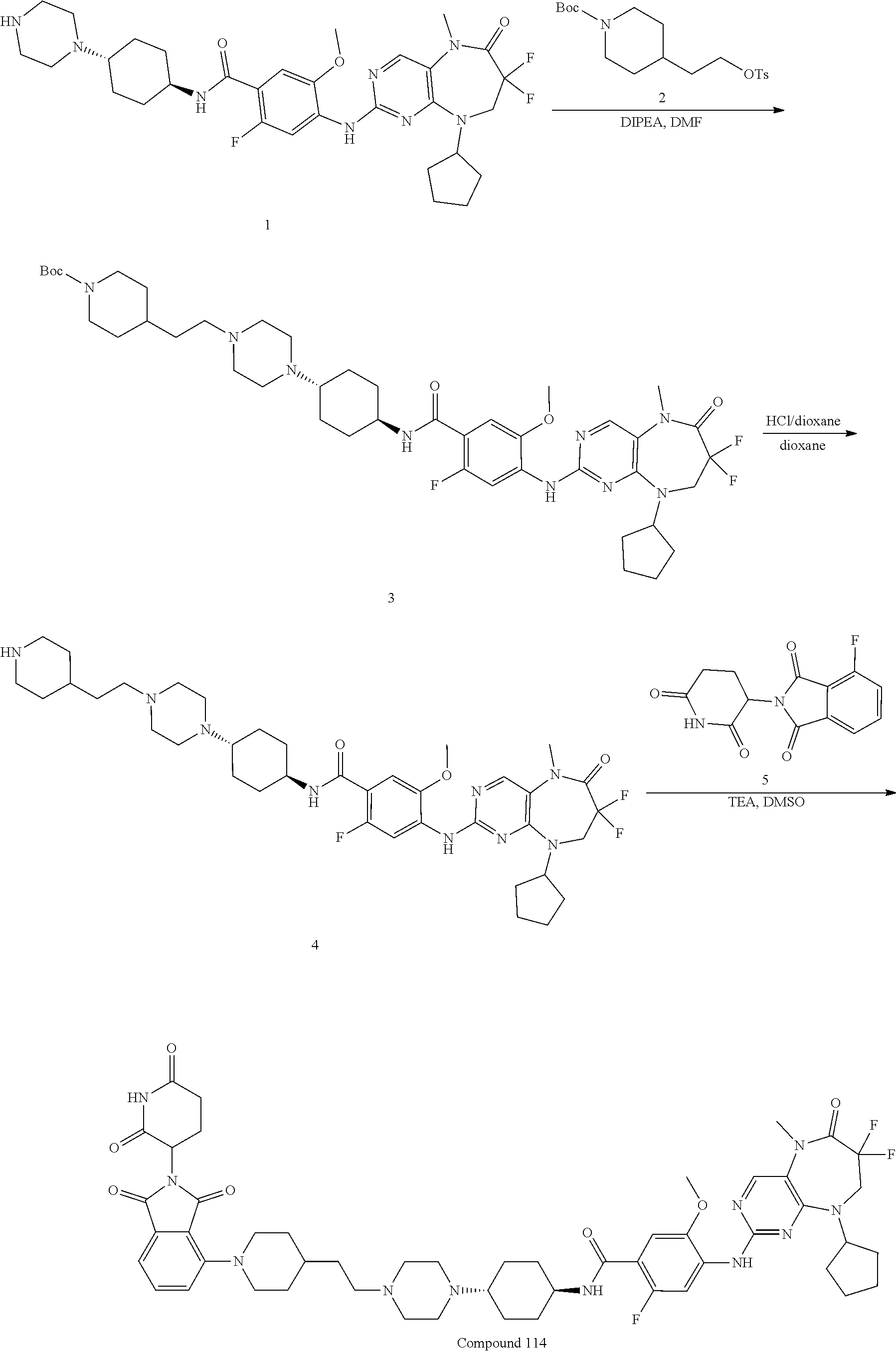

The compound 114 was synthesized by the method described in the scheme similar to the method described in Example 83.

MS(M+H)⁺=998.1, ¹H NMR (400 MHz, DMSO-d₆) δ=11.07 (br s, 1H), 8.29 (s, 1H), 8.24 (d, J=13.3 Hz, 1H), 8.02 (s, 1H), 7.80 (dd, J=3.6, 7.8 Hz, 1H), 7.67 (dd, J=7.3, 8.4 Hz, 1H), 7.32 (dd, J=5.1, 7.7 Hz, 2H), 7.18 (d, J=6.8 Hz, 1H), 5.08 (dd, J=5.4, 12.8 Hz, 1H), 4.87-4.76 (m, 1H), 4.13-4.01 (m, 2H), 3.91 (s, 3H), 3.73-3.62 (m, 3H), 3.30 (s, 3H), 2.93-2.80 (m, 3H), 2.63-2.52 (m, 8H), 2.37-2.34 (m, 1H), 2.31-2.25 (m, 2H), 2.24-2.15 (m, 1H), 2.06-1.87 (m, 5H), 1.86-1.69 (m, 6H), 1.68-1.22 (m, 14H).

### Example 115. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-((1-(2-(2,6-dioxopiperidin-3-yl)-1,3-dioxoisoindolin-4-yl)piperidin-4-yl)oxy)cyclohexyl)-2-fluoro-5-methoxybenzamide (Compound 115)

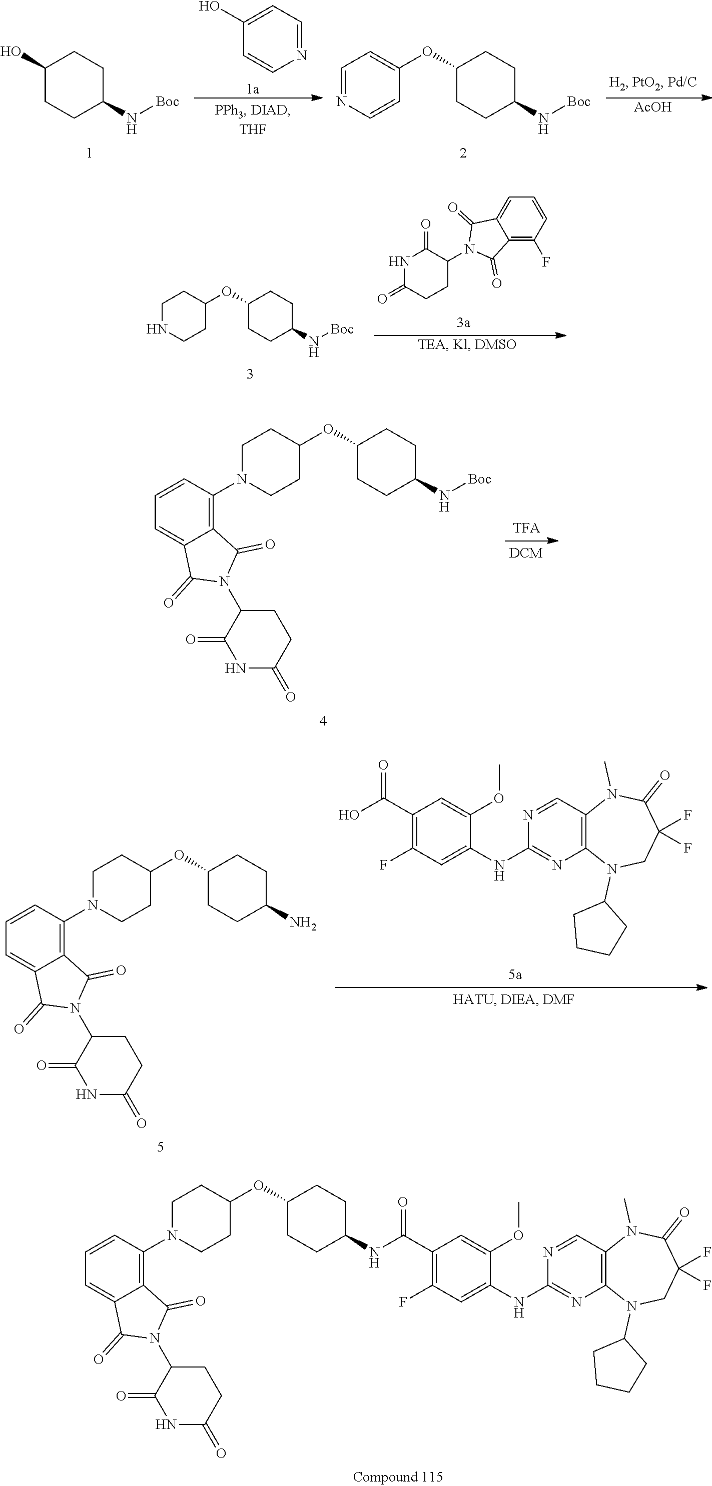

### Step 1. Synthesis of tert-butyl ((1r,4r)-4-(pyridin-4-yloxy)cyclohexyl)carbamate (2)

To a solution of tert-butyl ((1s,4s)-4-hydroxycyclohexyl)carbamate (3 g, 13.93 mmol), pyridin-4-ol (1.33 g, 13.93 mmol) and PPh₃ (5.48 g, 20.90 mmol) in THF (60 mL) was added DIAD (4.23 g, 20.90 mmol, 4.06 mL) at 0° C. The mixture was stirred at 25° C. for 12 hr. LCMS showed a peak (5%) with desired mass. The mixture was poured into cold HCl (0.5M, 80 mL) and washed with EtOAc (10 mL×3). The Na₂CO₃ aqueous solution was added to the aqueous layer to adjust pH to 9, and extracted with EtOAc (20 mL×4). The combined organic layers were dried over Na₂SO₄ and concentrated to afford tert-butyl ((1r,4r)-4-(pyridin-4-yloxy)cyclohexyl)carbamate (1 g, crude) as white solid, which was used for the next step directly. MS (M+H)⁺=293.1

### Step 2. Synthesis of tert-butyl ((1r,4r)-4-(piperidin-4-yloxy)cyclohexyl)carbamate (3)

To a solution of tert-butyl ((1r,4r)-4-(pyridin-4-yloxy)cyclohexyl)carbamate (1.1 g, 3.76 mmol) in AcOH (20 mL) was added Pd/C (300 mg, 3.76 mmol, 10% purity), PtO₂ (512.60 mg, 2.26 mmol). The mixture was stirred at 55° C. under H₂ (45 PSI) for 24 hr. LCMS showed the starting material was consumed completely and desired mass. The reaction mixture was filtered through a celite pad and the filtrate was concentrated. The crude product was dissolved with deionized water (40 mL) and lyophilized to afford tert-butyl N-[4-(4-piperidyloxy) cyclohexyl]carbamate (130 mg, crude, HOAC) as white solid, which was used for the next step directly. MS(M+H)⁺=299.2

### Step 3. Synthesis of tert-butyl ((1r,4r)-4-((1-(2-(2,6-dioxopiperidin-3-yl)-1,3-dioxoisoindolin-4-yl)piperidin-4-yl)oxy)cyclohexyl)carbamate (4)

A mixture of 2-(2,6-dioxopiperidin-3-yl)-4-fluoroisoindoline-1,3-dione (150.26 mg, 543.98 μmol), tert-butyl N-[4-(4-piperidyloxy) cyclohexyl]carbamate (130 mg, 362.65 μmol, HOAC), KI (25.15 mg, 151.43 μL) and TEA (110.09 mg, 1.09 mmol, 151.43 L) in DMSO (3 mL) was stirred at 80° C. for 16 hours. LCMS showed the tert-butyl N-[4-(4-piperidyloxy) cyclohexyl]carbamate was consumed completely and a peak (24%) with desired mass. The mixture was poured into water (80 mL) and extracted with EtOAc (20 mL×3). The combined organic phase was washed with brine (10 mL×3), dried over Na₂SO₄, filtered and concentrated under reduced pressure to give a residue. The residue was purified by flash silica gel chromatography (5 g SepaFlash® Silica Flash Column, Eluent of 0˜30% EtOAc/Petroleum ether gradient @60 mL/min) to afford tert-butyl ((1r,4r)-4-((1-(2-(2,6-dioxopiperidin-3-yl)-1,3-dioxoisoindolin-4-yl)piperidin-4-yl)oxy)cyclohexyl)carbamate (0.2 g, crude) as yellow oil, which was used for the next step directly. MS(M+H)⁺=555.2

### Step 4. Synthesis of 4-(4-(((1r,4r)-4-aminocyclohexyl)oxy)piperidin-1-yl)-2-(2,6-dioxopiperidin-3-yl)isoindoline-1,3-dione (6)

To a solution of ((1r,4r)-4-((1-(2-(2,6-dioxopiperidin-3-yl)-1,3-dioxoisoindolin-4-yl)piperidin-4-yl)oxy)cyclohexyl)carbamate (0.2 g, 360.60 μmol) in DCM (5 mL) was added TFA (1.54 g, 13.51 mmol, 1 mL) at 25° C. The resulting mixture was stirred at 25° C. for 0.5 hr. LCMS showed the starting material was consumed completely, and a peak (62%) with desired mass. The mixture was concentrated under reduced pressure to afford 4-(4-(((1r,4r)-4-aminocyclohexyl)oxy)piperidin-1-yl)-2-(2,6-dioxopiperidin-3-yl)isoindoline-1,3-dione (0.3 g, crude, TFA) as brown oil, which was used for the next step directly. MS(M+H)⁺=455.1

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-((1-(2-(2,6-dioxopiperidin-3-yl)-1,3-dioxoisoindolin-4-yl)piperidin-4-yl)oxy)cyclohexyl)-2-fluoro-5-methoxybenzamide (Compound 115)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (60 mg, 128.91 μmol) in DMF (1 mL) was added HATU (58.82 mg, 154.70 μmol) and DIPEA (99.97 mg, 773.49 μmol, 134.72 μL). The mixture was stirred at 25° C. for 10 min. To mixture was added 4-(4-(((1r,4r)-4-aminocyclohexyl)oxy)piperidin-1-yl)-2-(2,6-dioxopiperidin-3-yl)isoindoline-1,3-dione (102.61 mg, 180.48 μmol, TFA). The mixture was stirred at 25° C. for 2 h. LCMS showed 4-(4-(((1r,4r)-4-aminocyclohexyl)oxy)piperidin-1-yl)-2-(2,6-dioxopiperidin-3-yl)isoindoline-1,3-dione was consumed completely and a main peak with desired mass. The mixture solution was diluted with EtOAc (20 mL) concentrated under reduced pressure to give the residue. The residue was purified by flash silica gel chromatography (4 g SepaFlash® Silica Flash Column, Eluent of 0˜100% EtOAc/Petroleum ether gradient @60 mL/min; Eluent of 0˜50% Methanol/EtOAc @60 mL/min) to give crude product. The crude product was purified by prep-HPLC (column: Waters Xbridge 150×25 mm×5 um; mobile phase: [water(NH₄HCO₃)-ACN]; B %: 52%-82%, 8 min) and lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-((1-(2-(2,6-dioxopiperidin-3-yl)-1,3-dioxoisoindolin-4-yl)piperidin-4-yl)oxy)cyclohexyl)-2-fluoro-5-methoxybenzamide (50.7 mg, 54.53 μmol, 42.30% yield, 97% purity) as yellow solid. MS(M+H)⁺=902.5

¹H NMR (400 MHz, DMSO-d₆) δ=11.08 (br s, 1H), 8.29 (s, 1H), 8.24 (d, J=13.3 Hz, 1H), 8.03 (s, 1H), 7.85 (dd, J=3.4, 7.6 Hz, 1H), 7.68 (dd, J=7.3, 8.3 Hz, 1H), 7.33 (t, J=7.5 Hz, 2H), 7.18 (d, J=6.6 Hz, 1H), 5.10 (dd, J=5.4, 12.9 Hz, 1H), 4.82 (q, J=8.1 Hz, 1H), 4.07 (t, J=13.9 Hz, 2H), 3.91 (s, 3H), 3.81-3.60 (m, 2H), 3.59-3.48 (m, 2H), 3.44-3.38 (m, 1H), 3.33 (s, 3H), 3.07 (t, J=9.4 Hz, 2H), 2.95-2.80 (m, 1H), 2.64-2.53 (m, 2H), 2.05-1.82 (m, 9H), 1.76-1.55 (m, 8H), 1.45-1.22 (m, 4H).

### Example 116. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-((1-(2-(2,6-dioxopiperidin-3-yl)-1,3-dioxoisoindolin-5-yl)piperidin-4-yl)oxy)cyclohexyl)-2-fluoro-5-methoxybenzamide (Compound 116)

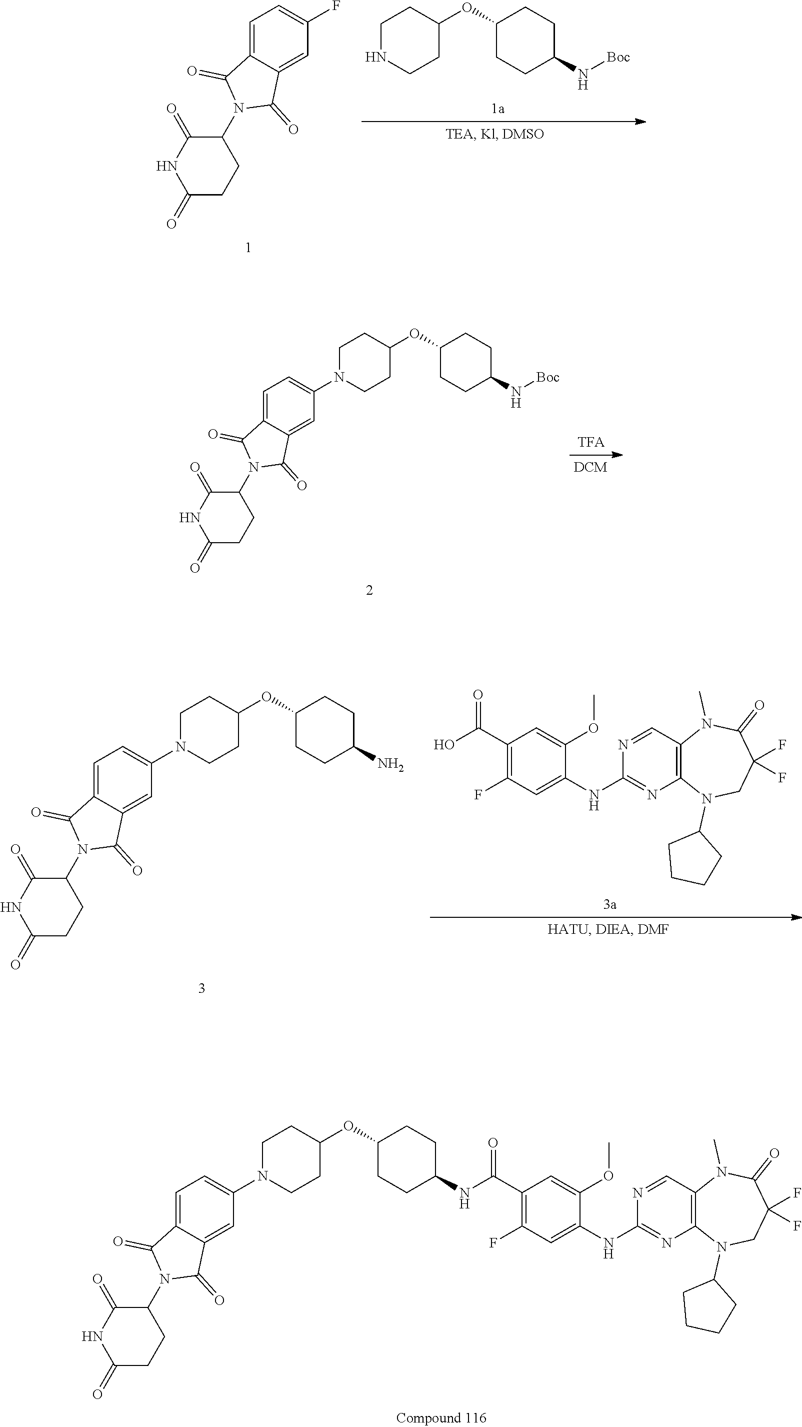 [SMILES: O=C1CCC(N2C(=O)c3ccc(F)cc3C2=O)C(=O)N1 ; CC(C)(C)OC(=O)N[C@H]1CC[C@H](OC2CCNCC2)CC1 ; CC(C)(C)OC(=O)N[C@H]1CC[C@H](OC2CCN(c3ccc4c(c3)C(=O)N(C3CCC(=O)NC3=O)C4=O)CC2)CC1 ; N[C@H]1CC[C@H](OC2CCN(c3ccc4c(c3)C(=O)N(C3CCC(=O)NC3=O)C4=O)CC2)CC1 ; COc1cc(C(=O)O)c(F)cc1Nc1ncc2c(n1)N(C1CCCC1)CC(F)(F)C(=O)N2C ; COc1cc(C(=O)N[C@H]2CC[C@H](OC3CCN(c4ccc5c(c4)C(=O)N(C4CCC(=O)NC4=O)C5=O)CC3)CC2)c(F)cc1Nc1ncc2c(n1)N(C1CCCC1)CC(F)(F)C(=O)N2C]

The compound 116 was synthesized by the method described in the scheme similar to the method described in Example 115.

MS(M+H)⁺=902.5, ¹H NMR (400 MHz, DMSO-d₆) δ=11.17-10.94 (m, 1H), 8.29 (s, 1H), 8.24 (d, J=13.3 Hz, 1H), 8.03 (s, 1H), 7.84 (dd, J=3.2, 7.3 Hz, 1H), 7.65 (d, J=8.5 Hz, 1H), 7.32 (s, 1H), 7.24 (dd, J=1.8, 8.7 Hz, 1H), 7.18 (d, J=6.6 Hz, 1H), 5.06 (dd, J=5.4, 12.9 Hz, 1H), 4.89-4.75 (m, 1H), 4.07 (t, J=13.8 Hz, 2H), 3.91 (s, 3H), 3.85-3.66 (m, 4H), 3.45-3.39 (m, 1H), 3.33-3.30 (m, 3H), 3.24 (t, J=10.1 Hz, 2H), 2.94-2.82 (m, 1H), 2.63-2.53 (m, 2H), 2.06-1.84 (m, 9H), 1.76-1.57 (m, 6H), 1.51-1.22 (m, 6H).

### Example 117. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(2-((1-(2-(2,6-dioxopiperidin-3-yl)-1,3-dioxoisoindolin-4-yl)piperidin-4-yl)methyl)-2-azaspiro[3.5]nonan-7-yl)-3-methoxybenzamide (Compound 117)

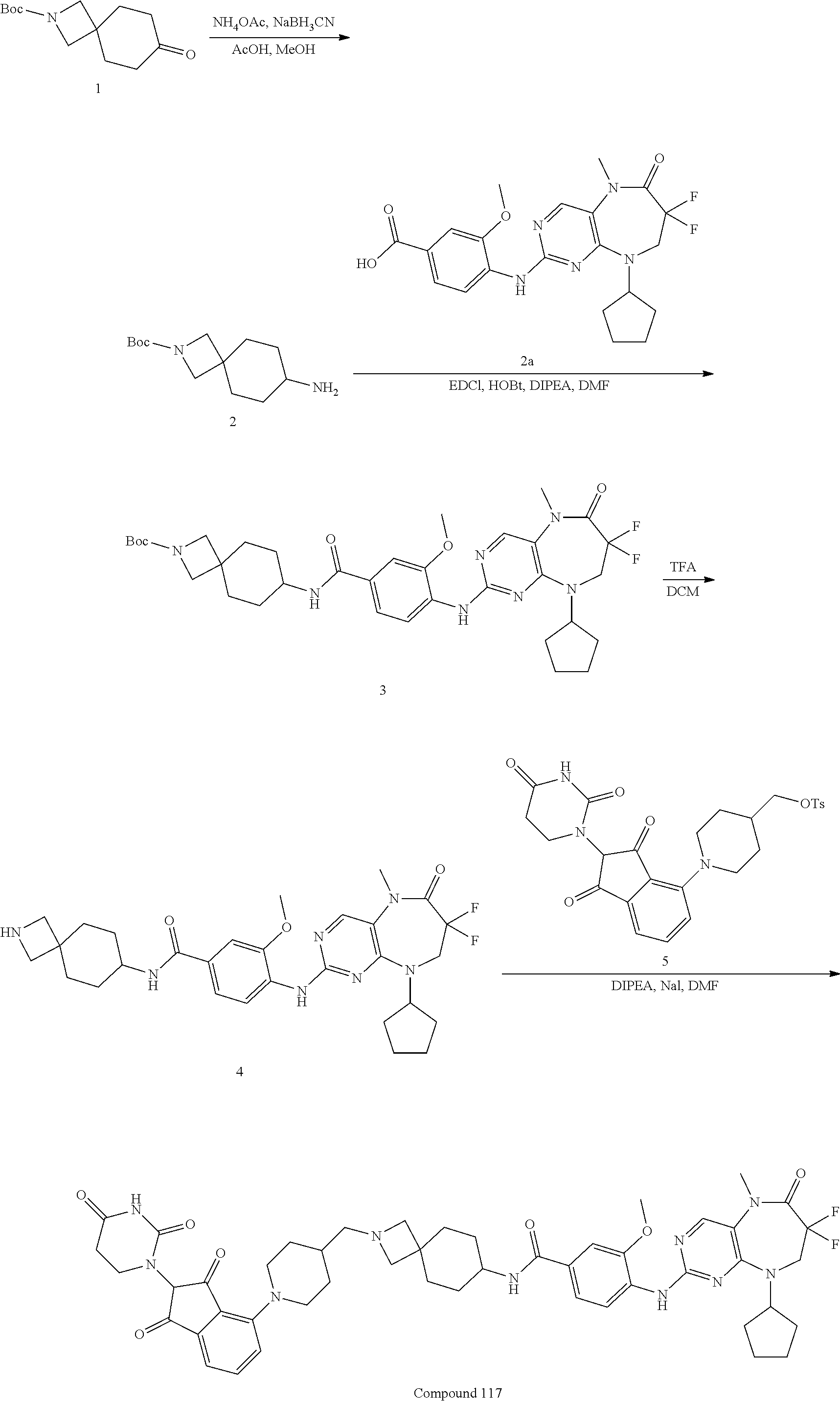

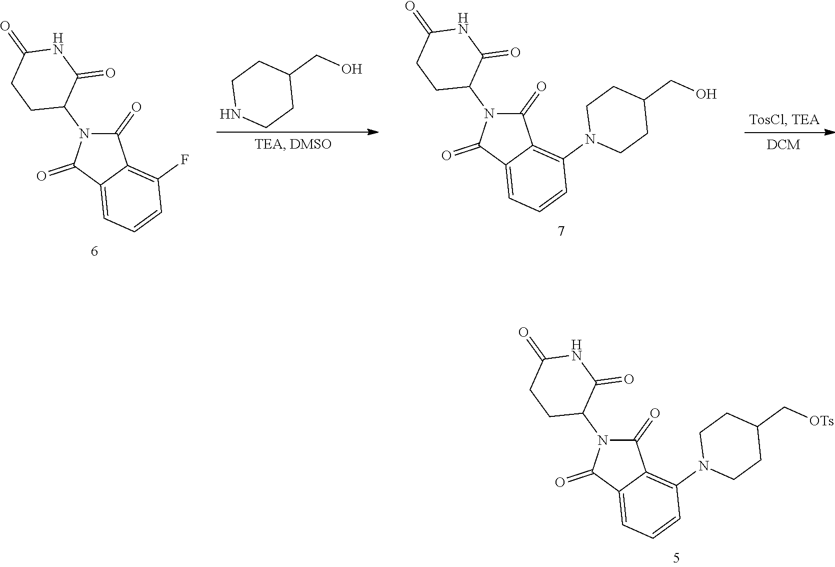 [SMILES: O=C1CCC(N2C(=O)c3cccc(F)c3C2=O)C(=O)N1 ; OCC1CCNCC1 ; O=C1CCC(N2C(=O)c3cccc(N4CCC(CO)CC4)c3C2=O)C(=O)N1 ; Cc1ccc(S(=O)(=O)OCC2CCN(c3cccc4c3C(=O)N(C3CCC(=O)NC3=O)C4=O)CC2)cc1]

### Step 1. Synthesis of 2-(2,6-dioxopiperidin-3-yl)-4-(4-(hydroxymethyl)piperidin-1-yl)isoindoline-1,3-dione (7)

To a solution of 2-(2,6-dioxopiperidin-3-yl)-4-fluoroisoindoline-1,3-dione (1 g, 3.62 mmol) in DMSO (10 mL) was added TEA (1.83 g, 18.10 mmol, 2.52 mL) and piperidin-4-ylmethanol (416.96 mg, 3.62 mmol) at 25° C. The mixture was stirred at 100° C. for 16 hr. LCMS showed the reagent 1 was consumed completely, and a main peak with desired mass. The mixture was poured into water (80 mL) and extracted with EtOAc (20 mL×3). The combined organic phase was dried over Na₂SO₄, filtered and concentrated under reduced pressure to afford 2-(2,6-dioxopiperidin-3-yl)-4-(4-(hydroxymethyl)piperidin-1-yl)isoindoline-1,3-dione (1.7 g, crude) as yellow oil, which was used for the next step directly. MS(M+H)⁺=372.1

### Step 2. Synthesis of (1-(2-(2,6-dioxopiperidin-3-yl)-1,3-dioxoisoindolin-4-yl)piperidin-4-yl)methyl 4-methylbenzenesulfonate (5)

To a solution 2-(2,6-dioxopiperidin-3-yl)-4-(4-(hydroxymethyl)piperidin-1-yl)isoindoline-1,3-dione (1.7 g, 4.58 mmol), TEA (1.39 g, 13.73 mmol, 1.91 mL) in DCM (40 mL) was added TosCl (1.31 g, 6.87 mmol) at 25° C. and the resulting mixture was stirred at 25° C. for 12 hr. LCMS showed reagent 1 was consumed completely, and a main peak with desired mass. The mixture was concentrated under reduced pressure. The crude product was purified by flash silica gel chromatography (25 g Sepa Flash® Silica Flash Column, Eluent of 0˜100% EtOAc/Petroleum ether gradient @100 mL/min) to afford (1-(2-(2,6-dioxopiperidin-3-yl)-1,3-dioxoisoindolin-4-yl)piperidin-4-yl)methyl 4-methylbenzenesulfonate (1.5 g, 2.65 mmol, 57.99% yield, 93% purity) as a yellow solid, which was used for the next step directly. MS(M+H)⁺=526.1

### Step 3. Synthesis of tert-butyl 7-amino-2-azaspiro[3.5]nonane-2-carboxylate (2)

To a solution of tert-butyl 7-oxo-2-azaspiro[3.5]nonane-2-carboxylate (300 mg, 1.25 mmol) in MeOH (15 mL) was added HOAc (75.28 mg, 1.25 mmol, 71.70 μL) and NH₄OAc (450.00 mg, 5.84 mmol). The mixture was stirred at 25° C. for 1 hr. Then NaBH₃CN (600.00 mg, 9.55 mmol) was added at 25° C. and the resulting mixture was stirred at 25° C. for 14 hr. LCMS showed a peak (21%) with the desired mass. The reaction mixture was added saturated Na₂CO₃ (sat. Aq, 15 mL) slowly at 0° C., and extracted with EtOAc 90 mL (30 mL×3). The combined organic layers were dried over anhydrous Na₂SO₄, filtered and the filtrate was concentrated under reduced pressure to afford tert-butyl 7-amino-2-azaspiro[3.5]nonane-2-carboxylate (300 mg, crude) as a light yellow oil. MS(M+H)⁺=241.2

### Step 4. Synthesis of tert-butyl 7-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)-2-azaspiro[3.5]nonane-2-carboxylate (3)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (550 mg, 1.23 mmol) in DMF (8 mL) were added EDCI (550.00 mg, 2.87 mmol), HOBt (320.83 mg, 2.37 mmol), DIPEA (680.17 mg, 5.26 mmol, 916.67 μL) and tert-butyl 7-amino-2-azaspiro[3.5]nonane-2-carboxylate (295.43 mg, 1.23 mmol) at 25° C. The mixture was stirred at 25° C. for 12 hr under N₂ atmosphere. LCMS showed a peak (51%) with the desired mass. The reaction mixture was diluted with H₂O (20 mL), and extracted with EtOAc (50 mL×2). The combined organic layers were washed with brine (30 mL×3), dried over anhydrous Na₂SO₄, filtered. The filtrate was concentrated under reduced pressure. The residue was purified by flash silica gel chromatography (Biotage; 20 g SepaFlash® Silica Flash Column, Eluent of 20˜80% EtOAc:Petroleum ether gradient, 60 mL/min) to afford tert-butyl 7-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)-2-azaspiro[3.5]nonane-2-carboxylate (500 mg, 746.54 μmol, 60.73% yield) as a light yellow solid. MS(M+H)⁺=670.4

¹H NMR (400 MHz, DMSO-d₆) δ=8.30-8.24 (m, 2H), 8.03 (br d, J=4.4 Hz, 1H), 7.96 (s, 1H), 7.54-7.44 (m, 2H), 4.84-4.71 (m, 1H), 4.13-4.01 (m, 2H), 3.94 (s, 3H), 3.79-3.70 (m, 1H), 3.61-3.48 (m, 4H), 3.33 (s, 3H), 3.18 (d, J=5.3 Hz, 1H), 1.97-1.83 (m, 4H), 1.79-1.68 (m, 4H), 1.62-1.49 (m, 5H), 1.39 (s, 9H), 1.37-1.31 (m, 2H).

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(2-azaspiro[3.5]nonan-7-yl)benzamide (4)

To a solution of tert-butyl 7-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)-2-azaspiro[3.5]nonane-2-carboxylate (0.1 g, 149.31 μmol) in DCM (5 mL) was added TFA (3.08 g, 27.01 mmol, 2.00 mL) at 25° C. and the resulting mixture was stirred at 25° C. for 0.5 hr. LCMS showed the starting material was consumed completely, and a main peak with desired mass. The mixture was concentrated under reduced pressure to afford 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(2-azaspiro[3.5]nonan-7-yl)benzamide (0.1 g, crude, TFA salt) as brown oil, which was used for the next step directly. MS(M+H)⁺=570.2

### Step 6. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(2-((1-(2-(2,6-dioxopiperidin-3-yl)-1,3-dioxoisoindolin-4-yl)piperidin-4-yl)methyl)-2-azaspiro[3.5]nonan-7-yl)-3-methoxybenzamide (Compound 117)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(2-azaspiro[3.5]nonan-7-yl)benzamide (0.1 g, 146.27 μmol, TFA salt) and (1-(2-(2,6-dioxopiperidin-3-yl)-1,3-dioxoisoindolin-4-yl)piperidin-4-yl)methyl 4-methylbenzenesulfonate (92.25 mg, 175.52 μmol) in DMF (1 mL) were added DIPEA (56.71 mg, 438.81 μmol, 76.43 μL) and NaI (4.38 mg, 29.25 μmol) at 25° C. The reaction mixture was heated to 60° C. for 16 hours. LCMS showed a peak (44%) with mass of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(2-azaspiro[3.5]nonan-7-yl)benzamide. Additional (1-(2-(2,6-dioxopiperidin-3-yl)-1,3-dioxoisoindolin-4-yl)piperidin-4-yl)methyl 4-methylbenzenesulfonate (46.13 mg, 87.76 μmol) was added and the resulting mixture was stirred at 60° C. for another 12 hrs. LCMS showed a peak (18%) with desired mass. The mixture was concentrated under reduced pressure. The residue was purified by flash silica gel chromatography (5 g SepaFlash® Silica Flash Column, Eluent of 0˜100% EtOAc/Petroleum ether gradient @60 mL/min; Eluent of 0˜50% Methanol/EtOAc @60 mL/min) followed by prep-HPLC (column: Phenomenex Synergi Polar-RP 100×25 mm×4 um; mobile phase: [water(TFA)-ACN]; B %: 35%-55%, 7 min) and the eluent was lyophilized. The residue was re-purified by prep-TLC (DCM:Methanol=10:1; Rf=0.4) and prep-HPLC (column: Waters Xbridge 150×25 mm×4 um; mobile phase: [water(NH₄HCO₃)-ACN]; B %: 45%-75%, 8 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(2-((1-(2-(2,6-dioxopiperidin-3-yl)-1,3-dioxoisoindolin-4-yl)piperidin-4-yl)methyl)-2-azaspiro[3.5]nonan-7-yl)-3-methoxybenzamide (11.5 mg, 12.21 μmol, 8.35% yield, 98% purity) as a yellow solid. MS(M+H)⁺=923.5

¹H NMR (400 MHz, DMSO-d₆) δ=11.04 (br s, 1H), 8.28-8.22 (m, 2H), 8.02 (br d, J=7.6 Hz, 1H), 7.94 (s, 1H), 7.55 (dd, J=7.0, 8.6 Hz, 1H), 7.51-7.44 (m, 2H), 7.14-7.04 (m, 2H), 5.11-5.01 (m, 1H), 4.83-4.70 (m, 1H), 4.04 (t, J=14.0 Hz, 2H), 3.93 (s, 3H), 3.80-3.67 (m, 1H), 3.66-3.54 (m, 2H), 3.53-3.43 (m, 1H), 3.32-3.32 (m, 3H), 2.99-2.77 (m, 6H), 2.62-2.53 (m, 2H), 2.43 (br t, J=6.8 Hz, 2H), 2.26-2.15 (m, 1H), 2.13-2.04 (m, 1H), 2.02-1.83 (m, 5H), 1.71 (br s, 4H), 1.65-1.54 (m, 5H), 1.49-1.30 (m, 6H).

### Example 118. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-fluorobenzamide (Compound 118)

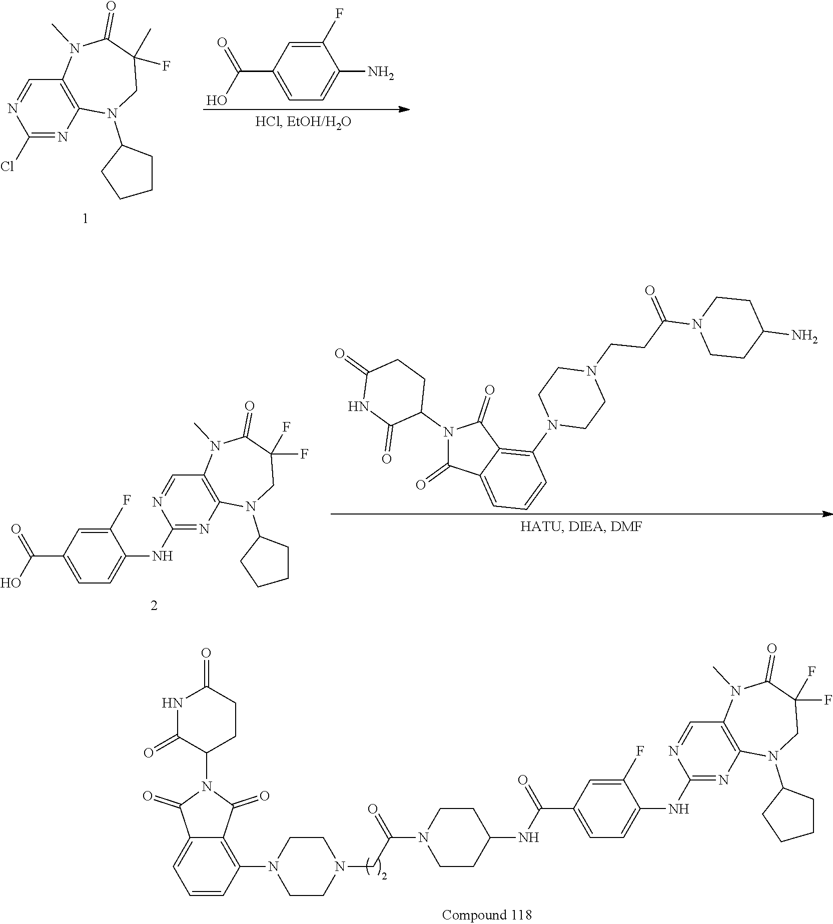

### Step 1. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-3-fluorobenzoic acid (2)

To a solution of 2-chloro-9-cyclopentyl-7,7-difluoro-5-methyl-5,7,8,9-tetrahydro-6H-pyrimido[4,5-b][1,4]diazepin-6-one (200 mg, 631.45 μmol) and 4-amino-3-fluoro-benzoic acid (127.34 mg, 820.88 μmol) in EtOH (3 mL) and H₂O (9 mL) was added HCl (12 M, 115.77 μL) at 20° C. and the resulting mixture was stirred at 100° C. for 32 h. LCMS showed 2-chloro-9-cyclopentyl-7,7-difluoro-5-methyl-5,7,8,9-tetrahydro-6H-pyrimido[4,5-b][1,4]diazepin-6-one was consumed completely and 79% peak with desired mass was detected. The reaction mixture was concentrated in vacuum. The crude product was triturated with EtOAc (1 mL) and ACN (1 mL) and DMF (1 mL) at 20° C. for 0.5 h and filtered. The filter cake was dried in vacuum to afford 4-((9-cyclopentyl-7,7-difluoro-5-methyl-6-oxo-6,7,8,9-tetrahydro-5H-pyrimido[4,5-b][1,4]diazepin-2-yl)amino)-3-fluorobenzoic acid (202 mg, 454.66 μmol, 72.00% yield, 98% purity) as a white solid. MS(M+H)⁺=436.1

### Step 2. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-fluorobenzamide (Compound 118)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-fluorobenzoic acid (80 mg, 183.74 μmol) in DMF (4 mL) were added HATU (76.85 mg, 202.11 μmol) and DIPEA (47.49 mg, 367.48 μmol, 64.01 μL). The mixture was stirred at 20° C. for 10 min and a solution of 4-(4-(3-(4-aminopiperidin-1-yl)-3-oxopropyl)piperazin-1-yl)-2-(2,6-dioxopiperidin-3-yl)isoindoline-1,3-dione (107.73 mg, 202.11 μmol, HCl) in DMF (4 mL) with DIPEA (47.49 mg, 367.48 μmol, 64.01 μL) was added and the resulting mixture was stirred at 20° C. for 1 h. LCMS showed starting material was consumed completely and 83% peak with desired mass was detected. The reaction mixture was diluted with H₂O (20 mL) and extracted with EtOAc (20 mL×3). The organic layer was washed with brine (20 mL×3), dried over Na₂SO₄, filtered and concentrated. The residue was purified by prep-HPLC (column: 3_Phenomenex Luna C18 75*30 mm*3 um; mobile phase: [water (TFA)-ACN]; B %: 24%-54%, 7 min) and re-purified by prep-HPLC (column: Waters Xbridge BEH C18 150*25 mm*5 um; mobile phase: [water(NH₄HCO₃)-ACN]; B %: 36%-66%, min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-fluorobenzamide (30.8 mg, 33.36 μmol, 18.16% yield, 99% purity) as a yellow solid. MS(M+H)⁺=914.1

¹H NMR (400 MHz, DMSO-d₆) δ=11.07 (br s, 1H), 9.07 (s, 1H), 8.28-8.18 (m, 2H), 8.01 (t, J=8.4 Hz, 1H), 7.76-7.62 (m, 3H), 7.40-7.30 (m, 2H), 5.09 (dd, J=5.4, 12.8 Hz, 1H), 4.74-4.63 (m, 1H), 4.37 (br d, J=12.8 Hz, 1H), 4.09-3.89 (m, 4H), 3.30 (br s, 7H), 3.13 (br t, J=12.1 Hz, 1H), 2.93-2.81 (m, 1H), 2.75-2.68 (m, 1H), 2.65-2.54 (m, 10H), 2.07-1.97 (m, 1H), 1.92-1.76 (m, 4H), 1.70-1.61 (m, 2H), 1.60-1.46 (m, 5H), 1.43-1.32 (m, 1H).

### Example 119. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-5-methoxy-2-(trifluoromethyl) benzamide (Compound 119)

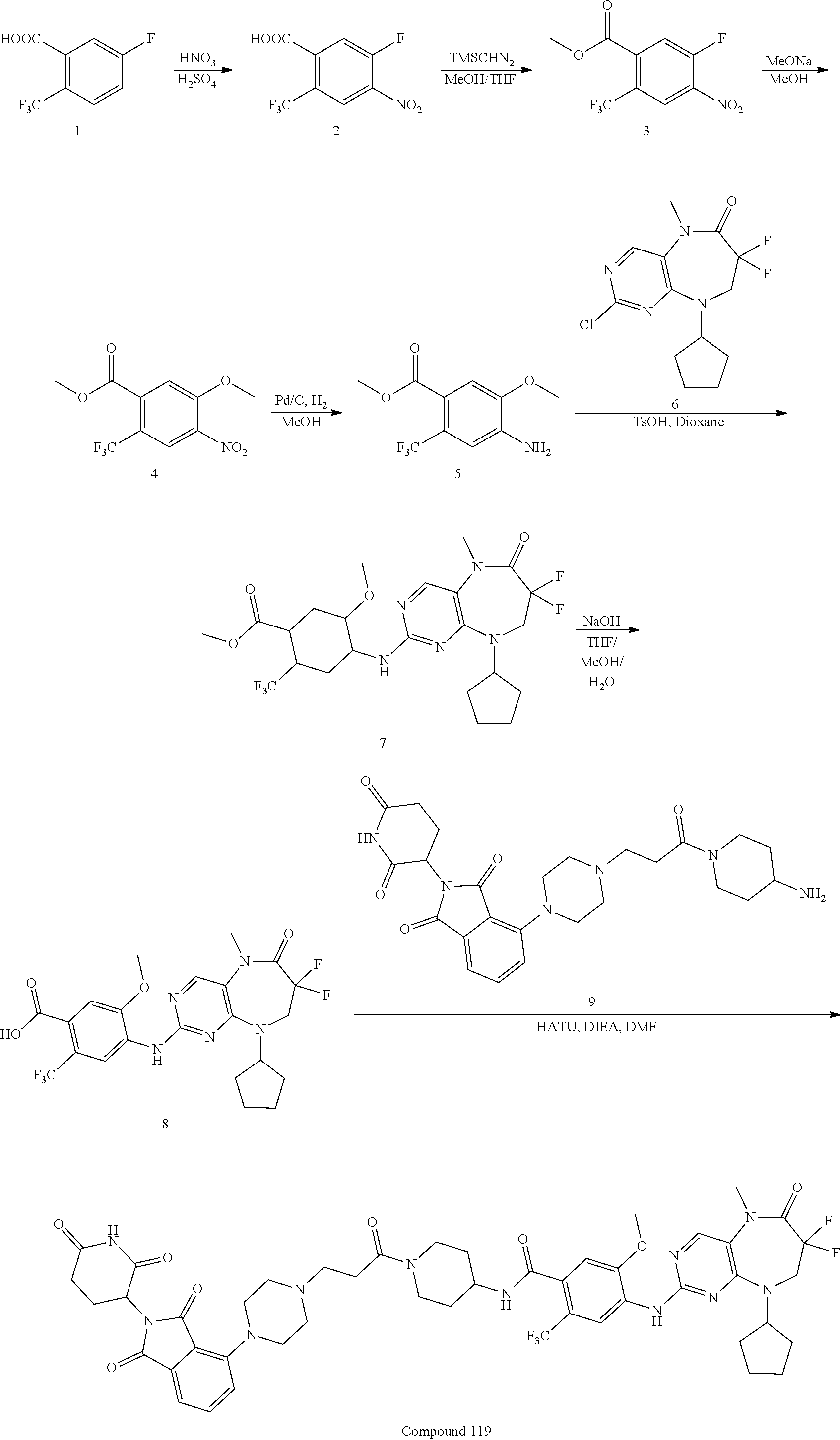

### Step 1. Synthesis of 5-fluoro-4-nitro-2-(trifluoromethyl)benzoic acid (2)

To the solution of 5-fluoro-2-(trifluoromethyl)benzoic acid (5 g, 24.03 mmol) in H₂SO₄ (25 mL) was added HNO₃ (35.00 g, 388.81 mmol, 25.00 mL, 70% purity) dropwise at 0° C. and the resulting mixture was stirred at 100° C. for 12 h. TLC (Petroleum ether/EtOAc=1/1) showed that most of started material was consumed and new spot formed. The reaction mixture was poured into ice water (300 mL) and extracted with EtOAc (150 mL×3). The combined organic layers were washed with brine (300 mL), dried over anhydrous sodium sulfate, filtered and concentrated under reduced pressure to afford 5-fluoro-4-nitro-2-(trifluoromethyl)benzoic acid (6 g, 11.85 mmol, crude) as yellow oil. MS(M+H)⁺=254.1

¹H NMR (400 MHz, CDCl₃) δ=10.69 (br s, 1H), 8.43 (d, J=6.6 Hz, 1H), 7.83 (d, J=10.1 Hz, 1H).

### Step 2. Synthesis of methyl 5-fluoro-4-nitro-2-(trifluoromethyl)benzoate (3)

To the solution of 5-fluoro-4-nitro-2-(trifluoromethyl)benzoic acid (6 g, 11.85 mmol) in MeOH (20 mL) and THF (80 mL) was added TMSCHN₂ (2 M, 17.78 mL) at 0° C. and the resulting mixture was stirred at 20° C. for 1 h. TLC (Petroleum ether/EtOAc=3/1) showed that 5-fluoro-4-nitro-2-(trifluoromethyl)benzoic acid was consumed and new spot formed. The reaction was concentrated. The residue was purified by flash silica gel chromatography (40 g SepaFlash® Silica Flash Column, Eluent of 0˜20% EtOAc/Petroleum ether gradient @80 mL/min) to afford methyl 5-fluoro-4-nitro-2-(trifluoromethyl)benzoate (2.9 g, crude) as yellow oil. MS(M+H)⁺=268.1

¹H NMR (400 MHz, CDCl₃) δ=8.47 (d, J=6.8 Hz, 1H), 7.75 (d, J=10.1 Hz, 1H), 3.99 (s, 3H).

### Step 3. Synthesis of methyl 5-methoxy-4-nitro-2-(trifluoromethyl)benzoate (4)

To the solution of methyl 5-fluoro-4-nitro-2-(trifluoromethyl)benzoate (1 g, 1.87 mmol) in MeOH (10 mL) was added NaOMe (5 M in MeOH, 1 mL) and the resulting mixture was stirred at 20° C. for 2 h. TLC (Petroleum ether/EtOAc=5/1) showed that 5-fluoro-4-nitro-2-(trifluoromethyl)benzoic acid was consumed and new spot formed. The mixture was poured into water (50 mL) and extracted with EtOAc (50 mL×3). The combined organic layer was washed by brine (100 mL), dried over Na₂SO₄, filtered and concentrated to afford methyl 5-methoxy-4-nitro-2-(trifluoromethyl)benzoate (1.1 g, crude) as yellow oil. MS(M+H)⁺=280.2

¹H NMR (400 MHz, CDCl₃) δ=8.22 (s, 1H), 7.48 (s, 1H), 4.07 (s, 3H), 3.92 (s, 3H).

### Step 4. Synthesis of methyl 4-amino-5-methoxy-2-(trifluoromethyl)benzoate (5)

To the solution of methyl 5-methoxy-4-nitro-2-(trifluoromethyl)benzoate (1.1 g, 1.97 mmol) in MeOH (20 mL) was added Pd/C (100 mg, 10% purity) under N₂ and the resulting mixture was stirred under H₂ (15 psi) at 20° C. for 12 h. TLC (Petroleum ether/EtOAc=5/1) showed the starting material was consumed and new spots formed. The mixture was filtered and concentrated. The residue was purified by flash silica gel chromatography (10 g SepaFlash® Silica Flash Column, Eluent of 0˜20% EtOAc/Petroleum ether gradient @80 mL/min) to afford methyl 4-amino-5-methoxy-2-(trifluoromethyl)benzoate (450 mg, 1.81 mmol, 91.66% yield) as a yellow solid. MS(M+H)⁺=235.2

¹H NMR (400 MHz, DMSO-d₆) δ=7.29 (s, 1H), 7.04 (s, 1H), 5.89 (s, 2H), 3.86 (s, 3H), 3.77 (s, 3H).

### Step 5. Synthesis of methyl 4-((9-cyclopentyl-7,7-difluoro-5-methyl-6-oxo-6,7,8,9-tetrahydro-5H-pyrimido[4,5-b][1,4]diazepin-2-yl)amino)-5-methoxy-2-(trifluoromethyl)benzoate (7)

To the solution of methyl 4-amino-5-methoxy-2-(trifluoromethyl)benzoate (0.35 g, 1.40 mmol) and 2-chloro-9-cyclopentyl-7,7-difluoro-5-methyl-5,7,8,9-tetrahydro-6H-pyrimido[4,5-b][1,4]diazepin-6-one (444.88 mg, 1.40 mmol) in dioxane (10 mL) was added TsOH (725.61 mg, 4.21 mmol) and the resulting mixture was stirred at 100° C. for 12 h. LCMS showed that methyl 4-amino-5-methoxy-2-(trifluoromethyl)benzoate was consumed and a peak (75%) with desired mass. The mixture was poured into brine (50 mL) and extracted with EtOAc (50 mL×3). The combined organic layer was dried over Na₂SO₄, filtered and concentrated. The residue was purified by silica gel column (Petroleum ether/EtOAc=1/1) to afford methyl 4-((9-cyclopentyl-7,7-difluoro-5-methyl-6-oxo-6,7,8,9-tetrahydro-5H-pyrimido[4,5-b][1,4]diazepin-2-yl)amino)-5-methoxy-2-(trifluoromethyl)benzoate (500 mg, 944.36 μmol, 67.23% yield, 100% purity) as a yellow solid. MS(M+H)⁺=530.1

### Step 6. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-5-methoxy-2-(trifluoromethyl)benzoic acid (8)

To the solution of methyl 4-((9-cyclopentyl-7,7-difluoro-5-methyl-6-oxo-6,7,8,9-tetrahydro-5H-pyrimido[4,5-b][1,4]diazepin-2-yl)amino)-5-methoxy-2-(trifluoromethyl)benzoate (250 mg, 472.18 μmol) in THF (2.5 mL), H₂O (2.5 mL) and MeOH (2.5 mL) was added NaOH (2 M in H₂O, 2.5 mL) and the resulting mixture was stirred at 25° C. for 12 h. LCMS showed that starting material was consumed and a peak (69%) with hydrate mass. The organic solvent was concentrated and the residue was adjusted pH=6 by 1 M HCl, the suspensions was filtered and collected filter cake to afford 4-((9-cyclopentyl-7,7-difluoro-5-methyl-6-oxo-6,7,8,9-tetrahydro-5H-pyrimido[4,5-b][1,4]diazepin-2-yl)amino)-5-methoxy-2-(trifluoromethyl)benzoic acid (180 mg, 349.22 μmol, 73.96% yield) as a yellow solid. MS(M+H₂O+H)⁺=534.1

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-5-methoxy-2-(trifluoromethyl) benzamide (Compound 119)

To the solution of 4-((9-cyclopentyl-7,7-difluoro-5-methyl-6-oxo-6,7,8,9-tetrahydro-5H-pyrimido[4,5-b][1,4]diazepin-2-yl)amino)-5-methoxy-2-(trifluoromethyl)benzoic acid (100 mg, 194.01 μmol) and 4-(4-(3-(4-aminopiperidin-1-yl)-3-oxopropyl)piperazin-1-yl)-2-(2,6-dioxopiperidin-3-yl)isoindoline-1,3-dione (103.41 mg, 194.01 μmol, HCl) in DMF (2 mL) were added HATU (110.65 mg, 291.02 μmol) and DIPEA (75.22 mg, 582.04 μmol, 101.38 μL) and the resulting mixture was stirred at 25° C. for 1 h. LCMS showed that 4-((9-cyclopentyl-7,7-difluoro-5-methyl-6-oxo-6,7,8,9-tetrahydro-5H-pyrimido[4,5-b][1,4]diazepin-2-yl)amino)-5-methoxy-2-(trifluoromethyl)benzoic acid was consumed and a peak (81%) with desired mass. The mixture was poured into water (20 mL) and extracted with EtOAc (20 mL×3). The combined organic layer was washed with brine (50 mL), dried over Na₂SO₄, filtered and concentrated. The resulting mixture was purified by prep-HPLC (column: Waters Xbridge 150*25 mm*5 μm; mobile phase: [water(NH₄HCO₃)-ACN]; B %: 40%-70%, 10 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-5-methoxy-2-(trifluoromethyl)benzamide (72 mg, 68.81 μmol, 35.47% yield, 95% purity) as a yellow solid. MS(M+H)⁺=994.2.

¹H NMR (400 MHz, DMSO-d₆) δ=11.09 (s, 1H), 8.58 (s, 1H), 8.40 (d, J=7.6 Hz, 1H), 8.29 (s, 1H), 8.11 (s, 1H), 7.74-7.65 (m, 1H), 7.35 (t, J=7.7 Hz, 2H), 7.11 (s, 1H), 5.09 (dd, J=5.3, 12.8 Hz, 1H), 4.92-4.73 (m, 1H), 4.36-4.21 (m, 1H), 4.05 (t, J=14.0 Hz, 2H), 4.00-3.85 (m, 5H), 3.35-3.28 (m, 6H), 3.22-3.14 (m, 1H), 2.93-2.76 (m, 2H), 2.64-2.53 (m, 11H), 2.07-1.81 (m, 5H), 1.73-1.64 (m, 2H), 1.63-1.50 (m, 4H), 1.46-1.29 (m, 2H).

### Example 120. Synthesis of 4-((9′-cyclopentyl-5′-methyl-6′-oxo-5′,6′,8′,9′-tetrahydrospiro[cyclopropane-1,7′-pyrimido[4,5-b][1,4]diazepin]-2′-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (Compound 120)

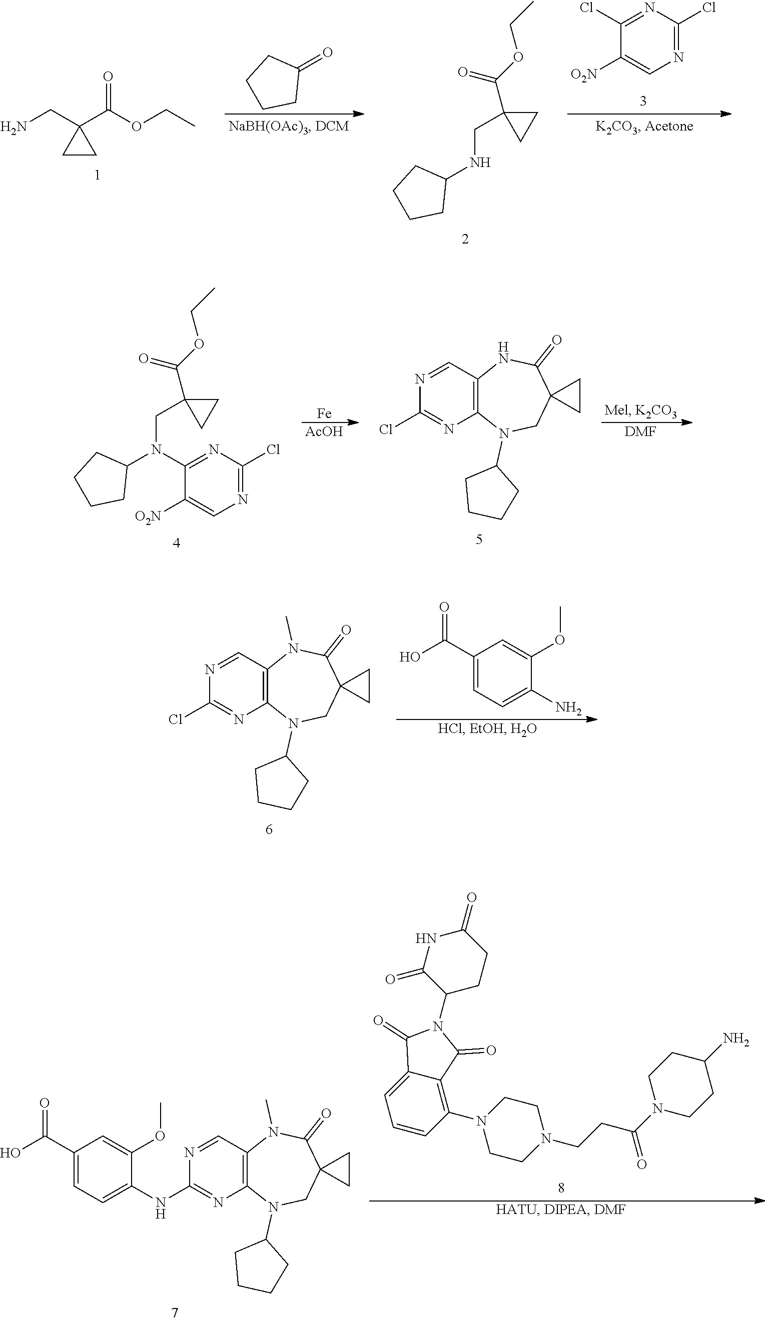

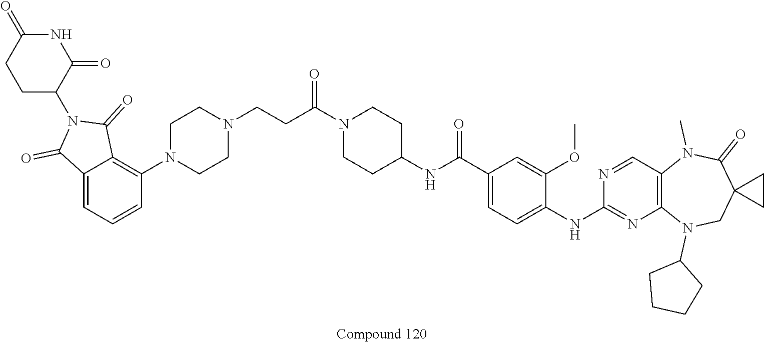 [SMILES: COc1cc(C(=O)NC2CCN(C(=O)CCN3CCN(c4cccc5c4C(=O)N(C4CCC(=O)NC4=O)C5=O)CC3)CC2)ccc1Nc1ncc2c(n1)N(C1CCCC1)CC1(CC1)C(=O)N2C]

### Step 1. Synthesis of ethyl 1-((cyclopentylamino)methyl)cyclopropane-1-carboxylate (2)

To a solution of ethyl 1-(aminomethyl)cyclopropane-1-carboxylate (1 g, 6.98 mmol) and cyclopentanone (587.47 mg, 6.98 mmol, 618.39 μL) in DCM (20 mL) was added NaBH(OAc (3.70 g 17.46 mmol). The mixture was stirred at 25° C. for 2 h. TLC (Petroleum ether:EtOAc=1:1) indicated ethyl 1-(aminomethyl)cyclopropane-1-carboxylate was consumed completely and one new spot was formed. The reaction mixture was diluted with water (200 mL) and extracted with EtOAc (50 mL×3). The combined organic layers were washed with brine (50 mL), dried over anhydrous sodium sulfate, filtered. The filtrate was adjusted to PH=4 with conc. HCl and concentrated under reduced pressure to afford ethyl 1-((cyclopentylamino)methyl)cyclopropane-1-carboxylate (350 mg, 1.41 mmol, 20.23% yield, HCl salt) as a white solid. MS(M+H)⁺=212.3

### Step 2. Synthesis of ethyl 1-(((2-chloro-5-nitropyrimidin-4-yl)(cyclopentyl)amino)methyl)cyclopropane-1-carboxylate (4)

To a solution of ethyl 1-((cyclopentylamino)methyl)cyclopropane-1-carboxylate (350 mg, 1.66 mmol) in ACETONE (5 mL) was added K₂CO₃ (686.78 mg, 4.97 mmol) and 2,4-dichloro-5-nitropyrimidine (321.30 mg, 1.66 mmol). The mixture was stirred at 25° C. for 3 h. LCMS showed a peak (11%) with desired mass. The reaction mixture was diluted with water (100 mL) and extracted with EtOAc (30 mL×3). The combined organic layers were washed with brine (30 mL), dried over anhydrous sodium sulfate, filtered. The filtrate was concentrated under reduced pressure. The residue was purified by column chromatography (SiO₂, Petroleum ether/EtOAc=1/0 to 100/5) to afford ethyl 1-(((2-chloro-5-nitropyrimidin-4-yl)(cyclopentyl)amino)methyl)cyclopropane-1-carboxylate (250 mg, 671.07 μmol, 40.51% yield, 99% purity) as a white solid. MS(M+H)⁺=369.1

### Step 3. Synthesis of 2′-chloro-9′-cyclopentyl-8′,9′-dihydrospiro[cyclopropane-1,7′-pyrimido[4,5-b][1,4]diazepin]-6′(5′H)-one (5)

To a solution of ethyl 1-(((2-chloro-5-nitropyrimidin-4-yl)(cyclopentyl)amino)methyl)cyclopropane-1-carboxylate (200 mg, 542.28 μmol) in AcOH (4 mL) was added Fe (151.42 mg, 2.71 mmol). The mixture was stirred at 60° C. for 2 h. LCMS showed ethyl 1-(((2-chloro-5-nitropyrimidin-4-yl)(cyclopentyl)amino)methyl)cyclopropane-1-carboxylate was consumed completely and one main peak with desired mass. The reaction mixture was filtered and filter cake was washed with EtOAc (50 mL). The filtrate was washed with brine (20 mL), dried over anhydrous sodium sulfate, filtered and concentrated. The residue was purified by column chromatography (SiO₂, Petroleum ether/EtOAc=5/1 to 3/1) to afford 2′-chloro-9′-cyclopentyl-8′,9′-dihydrospiro[cyclopropane-1,7′-pyrimido[4,5-b][1,4]diazepin]-6′(5′H)-one (150 mg, 507.24 μmol, 93.54% yield, 99% purity) as a white solid. MS(M+H)⁺=293.2.

### Step 4. Synthesis of 2′-chloro-9′-cyclopentyl-5′-methyl-8′,9′-dihydrospiro[cyclopropane-1,7′-pyrimido[4,5-b][1,4]diazepin]-6′(5′H)-one (6)

To a solution of 2′-chloro-9′-cyclopentyl-8′,9′-dihydrospiro[cyclopropane-1,7′-pyrimido[4,5-b][1,4]diazepin]-6′(5′H)-one (120 mg, 409.89 μmol) in DMF (3 mL) were added K₂CO₃ (141.62 mg, 1.02 mmol) and MeI (87.27 mg, 614.83 μmol, 38.28 μL). The mixture was stirred at 10° C. for 2 h. LCMS showed 2′-chloro-9′-cyclopentyl-8′,9′-dihydrospiro[cyclopropane-1,7′-pyrimido[4,5-b][1,4]diazepin]-6′(5′H)-one was consumed completely and one main peak with desired mass. The reaction mixture was diluted with water (50 mL) and extracted with EtOAc (20 mL×3). The combined organic layers were washed with brine (20 mL), dried over anhydrous sodium sulfate, filtered and concentrated under reduced pressure. The residue was purified by column chromatography (SiO₂, Petroleum ether/EtOAc=3/1 to 1/1) to afford 2′-chloro-9′-cyclopentyl-5′-methyl-8′,9′-dihydrospiro[cyclopropane-1,7′-pyrimido[4,5-b][1,4]diazepin]-6′(5′H)-one (100 mg, 322.70 μmol, 78.73% yield, 99% purity) as a white solid. MS(M+H)⁺=307.2

### Step 5. Synthesis of 4-((9′-cyclopentyl-5′-methyl-6′-oxo-5′,6′,8′,9′-tetrahydrospiro[cyclopropane-1,7′-pyrimido[4,5-b][1,4]diazepin]-2′-yl)amino)-3-methoxybenzoic acid (7)

A mixture of 2′-chloro-9′-cyclopentyl-5′-methyl-8′,9′-dihydrospiro[cyclopropane-1,7′-pyrimido[4,5-b][1,4]diazepin]-6′(5′H)-one (100 mg, 325.96 μmol), 4-amino-3-methoxybenzoic acid (54.49 mg, 325.96 μmol), HCl (12 M, 59.76 μL) in H₂O (3 mL) and EtOH (1 mL) was stirred at 100° C. for 10 h. LCMS showed 2′-chloro-9′-cyclopentyl-5′-methyl-8′,9′-dihydrospiro[cyclopropane-1,7′-pyrimido[4,5-b][1,4]diazepin]-6′(5′H)-one was consumed completely, and a peak (˜73%) with desired mass. The reaction mixture was concentrated under reduced pressure to afford 4-((9′-cyclopentyl-5′-methyl-6′-oxo-5′,6′,8′,9′-tetrahydrospiro[cyclopropane-1,7′-pyrimido[4,5-b][1,4]diazepin]-2′-yl)amino)-3-methoxybenzoic acid (100 mg, crude) as a gray solid. MS(M+H)⁺=438.3

### Step 6. Synthesis of 4-((9′-cyclopentyl-5′-methyl-6′-oxo-5′,6′,8′,9′-tetrahydrospiro[cyclopropane-1,7′-pyrimido[4,5-b][1,4]diazepin]-2′-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (Compound 120)

To a solution of 4-((9′-cyclopentyl-5′-methyl-6′-oxo-5′,6′,8′,9′-tetrahydrospiro[cyclopropane-1,7′-pyrimido[4,5-b][1,4]diazepin]-2′-yl)amino)-3-methoxybenzoic acid (80 mg, 182.86 μmol) and 4-(4-(3-(4-aminopiperidin-1-yl)-3-oxopropyl)piperazin-1-yl)-2-(2,6-dioxopiperidin-3-yl)isoindoline-1,3-dione (97.47 mg, 182.86 μmol, HCl salt) in DMF (3 mL) were added HATU (104.29 mg, 274.29 μmol) and DIPEA (70.90 mg, 548.58 μmol, 95.55 μL). The mixture was stirred at 25° C. for 2 h. LCMS showed 4-((9′-cyclopentyl-5′-methyl-6′-oxo-5′,6′,8′,9′-tetrahydrospiro[cyclopropane-1,7′-pyrimido[4,5-b][1,4]diazepin]-2′-yl)amino)-3-methoxybenzoic acid was consumed completely, and a peak (71%) with desired mass. The reaction mixture was diluted with H₂O (50 mL) and extracted with EtOAc (20 mL×3). The combined organic layers were washed with brine (20 ml), dried over anhydrous sodium sulfate, filtered. The filtrate was concentrated under reduced pressure. The residue was purified by column chromatography (SiO₂, MeOH/EtOAc=0/1 to 1/10) followed by prep-HPLC (neutral condition: column: Waters Xbridge 150×25 mm×5 μm; mobile phase: [water (NH₄HCO₃)-ACN]; B %: 36%-66%, 10 min) to afford 4-((9′-cyclopentyl-5′-methyl-6′-oxo-5′,6′,8′,9′-tetrahydrospiro[cyclopropane-1,7′-pyrimido[4,5-b][1,4]diazepin]-2′-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (30.4 mg, 32.52 μmol, 17.79% yield, 98% purity) as a yellow solid. MS(M+H)⁺=916.1

¹H NMR (400 MHz, DMSO-d₆) δ=11.09 (s, 1H), 8.41 (d, J=8.3 Hz, 1H), 8.10-8.05 (m, 1H), 7.99 (s, 1H), 7.79-7.67 (m, 2H), 7.53-7.45 (m, 2H), 7.43-7.32 (m, 2H), 5.11 (dd, J=5.4, 12.7 Hz, 1H), 4.93-4.82 (m, 1H), 4.47-4.36 (m, 1H), 4.14-4.02 (m, 1H), 3.95 (s, 4H), 3.48 (s, 2H), 3.32-3.30 (m, 4H), 3.17 (s, 4H), 2.97-2.83 (m, 2H), 2.77-2.71 (m, 1H), 2.66-2.52 (m, 9H), 2.09-2.00 (m, 1H), 1.94-1.82 (m, 4H), 1.74-1.66 (m, 2H), 1.64-1.57 (m, 2H), 1.56-1.40 (m, 4H), 0.93-0.89 (m, 2H), 0.70-0.65 (m, 2H).

### Example 121. Synthesis of 4-((9-cyclopentyl-5,7,7-trimethyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (Compound 121)

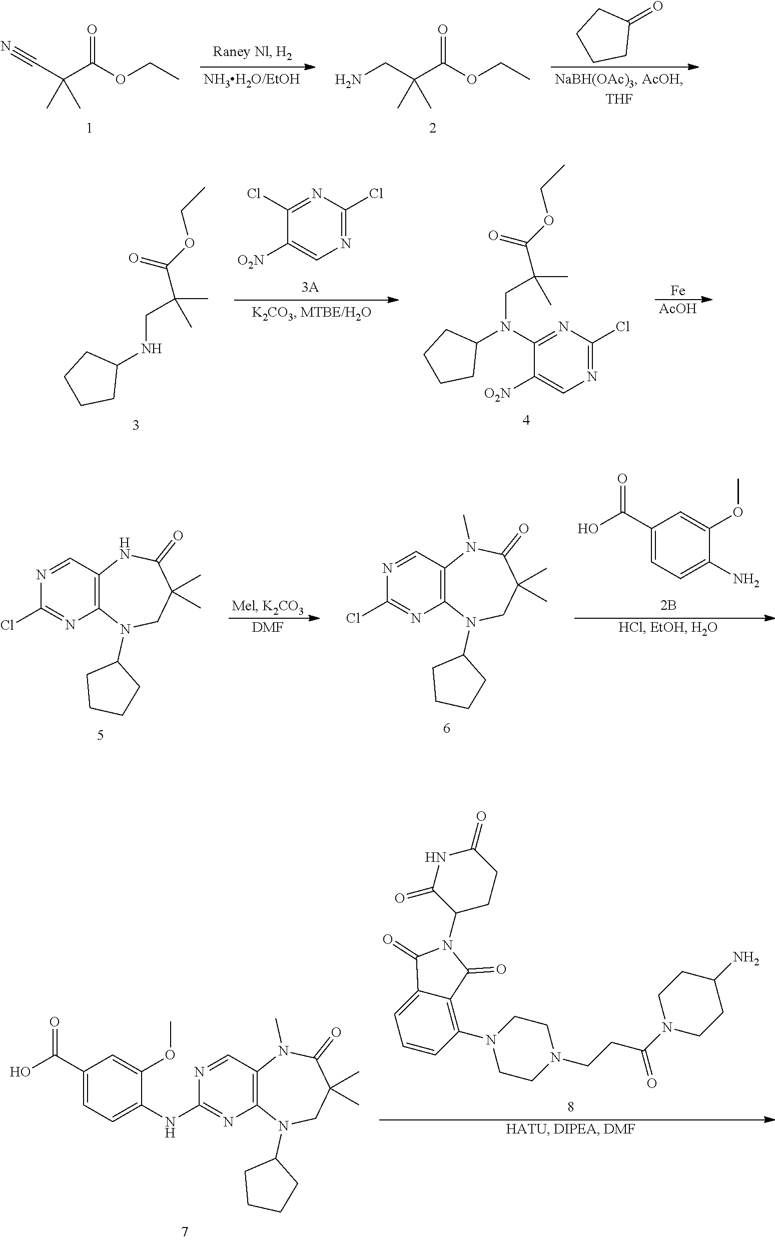

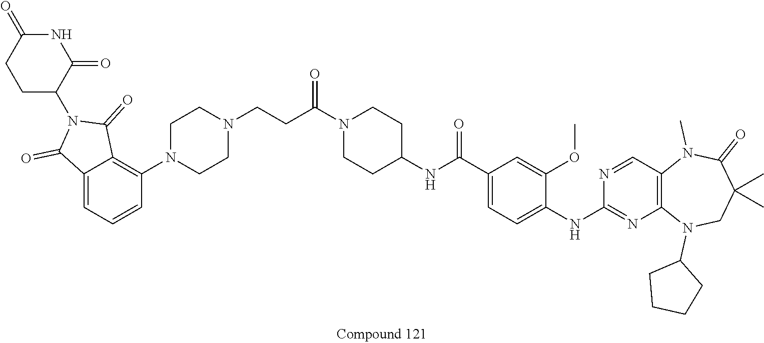 [SMILES: COc1cc(C(=O)NC2CCN(C(=O)CCN3CCN(c4cccc5c4C(=O)N(C4CCC(=O)NC4=O)C5=O)CC3)CC2)ccc1Nc1ncc2c(n1)N(C1CCCC1)CC(C)(C)C(=O)N2C]

### Step 1. Synthesis of ethyl 3-amino-2,2-dimethylpropanoate (2)

To a solution of ethyl 2-cyano-2-methyl-propanoate (15 g, 106.26 mmol) in EtOH (150 mL) and NH₃·H₂O (15 mL) was added Raney-Ni (18.21 g, 212.51 mmol) under N₂ atmosphere. The suspension was degassed and purged with H₂ for 3 times. The mixture was stirred at 20° C. for 16 h under H₂ (50 Psi). TLC (SiO₂, Petroleum ether:EtOAc=2:1) indicated starting material was consumed completely and one new spot was detected. The reaction mixture was diluted with EtOH (500 mL) and filtered. The filtrate was concentrated in vacuum to afford ethyl 3-amino-2,2-dimethylpropanoate (13.6 g, crude) as a colorless oil. MS(M+H)⁺=146.2

### Step 2. Synthesis of ethyl 3-(cyclopentylamino)-2,2-dimethylpropanoate (3)

To a solution of ethyl 3-amino-2,2-dimethylpropanoate (12.6 g, 86.78 mmol) and cyclopentanone (8.76 g, 104.13 mmol, 9.22 mL) in THF (126 mL) and AcOH (12.6 mL) was added NaBH(OAc)₃ (27.59 g, 130.17 mmol) at 20° C. and the resulting mixture was stirred at 20° C. for 16 h. LCMS showed starting material was consumed completely and desired mass. TLC (SiO₂, Petroleum ether:EtOAc=3:1) indicated starting material was consumed completely and two new spots were formed. The reaction mixture was diluted with H₂O (150 mL) and extracted with EtOAc (150 mL×3). The combined organic layers were washed with brine (150 mL×3), dried over Na₂SO₄, filtered and concentrated to afford ethyl 3-(cyclopentylamino)-2,2-dimethylpropanoate (7.6 g, 35.63 mmol, 41.06% yield) as a white solid. MS(M+H)⁺=214.3

### Step 3. Synthesis of ethyl 3-((2-chloro-5-nitropyrimidin-4-yl)(cyclopentyl)amino)-2,2-dimethylpropanoate (4)

To a solution of ethyl 3-(cyclopentylamino)-2,2-dimethylpropanoate (6.6 g, 30.94 mmol) and 2,4-dichloro-5-nitro-pyrimidine (9.00 g, 46.41 mmol) in MTBE (70 mL) was added a solution of K₂CO₃ (17.10 g, 123.76 mmol) in H₂O (35 mL) drop-wise at 0° C. and the resulting mixture was stirred at 20° C. for 16 h. LCMS showed a peak (60%) with desired mass. The reaction mixture was combined with another batch (1 g scale) for work-up. The reaction mixture was diluted with H₂O (400 mL) and extracted with EtOAc (400 mL×3). The organic layer was dried over Na₂SO₄, filtered and concentrated to afford ethyl 3-((2-chloro-5-nitropyrimidin-4-yl)(cyclopentyl)amino)-2,2-dimethylpropanoate (8.6 g, crude) as a yellow oil. MS(M+H)⁺=371.1

### Step 4. Synthesis of 2-chloro-9-cyclopentyl-7,7-dimethyl-5,7,8,9-tetrahydro-6H-pyrimido[4,5-b][1,4]diazepin-6-one (5)

To a solution of ethyl 3-((2-chloro-5-nitropyrimidin-4-yl)(cyclopentyl)amino)-2,2-dimethylpropanoate (8.6 g, 23.19 mmol) in AcOH (80 mL) was added Fe (5.18 g, 92.76 mmol) at 20° C. and the resulting mixture was stirred at 60° C. for 2 h. LCMS showed starting material was consumed completely and a peak (88%) with desired mass. The reaction mixture was concentrated in vacuum. The residue was diluted with H₂O (100 mL) and extracted with EtOAc (100 mL×3). The organic layer was dried over Na₂SO₄, filtered and concentrated in vacuum to afford 2-chloro-9-cyclopentyl-7,7-dimethyl-5,7,8,9-tetrahydro-6H-pyrimido[4,5-b][1,4]diazepin-6-one (7.2 g, crude) as a yellow solid. MS(M+H)⁺=295.2

### Step 5. Synthesis of 2-chloro-9-cyclopentyl-5,7,7-trimethyl-5,7,8,9-tetrahydro-6H-pyrimido[4,5-b][1,4]diazepin-6-one (6)

To a solution of 2-chloro-9-cyclopentyl-7,7-dimethyl-5,7,8,9-tetrahydro-6H-pyrimido[4,5-b][1,4]diazepin-6-one (7.2 g, 24.43 mmol) in DMF (80 mL) were added K₂CO₃ (10.13 g, 73.28 mmol) and MeI (4.16 g, 29.31 mmol, 1.82 mL) at 20° C. and the resulting mixture was stirred at 20° C. for 4 h. LCMS showed a peak (23%) of starting material remained and a peak (53%) with desired mass. Additional MeI (2.08 g, 14.66 mmol, 912.33 μL) was added and the resulting mixture was stirred at 20° C. for 16 h. LCMS showed starting material was consumed completely and a peak (56%) with desired mass. The reaction mixture was diluted with H₂O (160 mL) and extracted with EtOAc (160 mL×3). The organic layer was washed with brine (160 mL×3), dried over Na₂SO₄, filtered and concentrated. The residue was purified by flash silica gel chromatography (40 g SepaFlash® Silica Flash Column, Eluent of 0˜15% EtOAc/Petroleum ether gradient @100 mL/min) to afford 2-chloro-9-cyclopentyl-5,7,7-trimethyl-5,7,8,9-tetrahydro-6H-pyrimido[4,5-b][1,4]diazepin-6-one (2.7 g, 8.39 mmol, 34.36% yield, 96% purity) as an orange solid. MS(M+H)⁺=309.2

### Step 6. Synthesis of 4-((9-cyclopentyl-5,7,7-trimethyl-6-oxo-6,7,8,9-tetrahydro-5H-pyrimido[4,5-b][1,4]diazepin-2-yl)amino)-3-methoxybenzoic acid (7)

To a solution of 2-chloro-9-cyclopentyl-5,7,7-trimethyl-5,7,8,9-tetrahydro-6H-pyrimido[4,5-b][1,4]diazepin-6-one (300 mg, 971.48 μmol) and 4-amino-3-methoxy-benzoic acid (211.11 mg, 1.26 mmol) in EtOH (3 mL) and H₂O (9 mL) was added HCl (12 M, 178.11 μL) at 20° C. and the resulting mixture was stirred at 100° C. for 16 h. LCMS showed starting material was consumed completely and a peak (68%) with desired mass. The reaction mixture was concentrated in vacuum. The crude product was triturated with a mixture solvent (EtOAc/EtOH/DMF (9 mL, 1/1/1)) at 25° C. for 0.5 h and filtered. The filter cake was dried in vacuum to afford 4-((9-cyclopentyl-5,7,7-trimethyl-6-oxo-6,7,8,9-tetrahydro-5H-pyrimido[4,5-b][1,4]diazepin-2-yl)amino)-3-methoxybenzoic acid (286 mg, 598.67 μmol, 61.62% yield, 92% purity) as an off-white solid. MS(M+H)⁺=440.2

### Step 7. Synthesis of 4-((9-cyclopentyl-5,7,7-trimethyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (Compound 121)

To a solution of 4-((9-cyclopentyl-5,7,7-trimethyl-6-oxo-6,7,8,9-tetrahydro-5H-pyrimido[4,5-b][1,4]diazepin-2-yl)amino)-3-methoxybenzoic acid (80 mg, 182.02 μmol) in DMF (2 mL) were added HATU (76.13 mg, 200.22 μmol) and DIPEA (47.05 mg, 364.04 μmol, 63.41 μL). The mixture was stirred at 20° C. for 10 min and a solution of 4-(4-(3-(4-aminopiperidin-1-yl)-3-oxopropyl)piperazin-1-yl)-2-(2,6-dioxopiperidin-3-yl)isoindoline-1,3-dione (97.02 mg, 182.02 μmol, HCl) in DMF (2 mL) with DIPEA (47.05 mg, 364.04 μmol, 63.41 μL) was added and the resulting mixture was stirred at 20° C. for 12 h. LCMS showed all starting material was consumed completely and a peak (73%) with desired mass. The reaction mixture was diluted with H₂O (15 mL) and extracted with EtOAc (15 mL×3). The organic layer was washed with brine (15 mL×3), dried over Na₂SO₄, filtered and concentrated. The residue was purified by prep-HPLC (column: Unisil 3-100 C18 Ultra 150*50 mm*3 μm; mobile phase: [water(FA)-ACN]; B %: 8%-38%, 10 min) and the eluent was lyophilized to afford 4-((9-cyclopentyl-5,7,7-trimethyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-4-yl)-3-methoxybenzamide (78 mg, 80.10 μmol, 44.00% yield, 99% purity, FA) as a yellow solid. MS(M+H)⁺=918.2

¹H NMR (400 MHz, DMSO-d₆) δ=11.10 (s, 1H), 8.37 (d, J=8.3 Hz, 1H), 8.29 (s, 1H), 8.14 (br d, J=7.6 Hz, 1H), 7.98 (s, 1H), 7.74-7.66 (m, 2H), 7.51-7.44 (m, 2H), 7.35 (t, J=7.4 Hz, 2H), 5.23-5.13 (m, 1H), 5.09 (dd, J=5.5, 12.7 Hz, 1H), 4.40 (br d, J=11.9 Hz, 1H), 4.11-4.00 (m, 1H), 3.94 (s, 3H), 3.30 (br s, 6H), 3.21-3.08 (m, 5H), 2.93-2.81 (m, 1H), 2.66-2.54 (m, 10H), 2.08-1.96 (m, 1H), 1.93-1.78 (m, 4H), 1.77-1.67 (m, 2H), 1.61 (br s, 4H), 1.52-1.33 (m, 2H), 1.09 (s, 6H).

### Example 122. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(2-(2,6-dioxopiperidin-3-yl)-1,3-dioxoisoindolin-4-yl)piperazin-1-yl)acetyl)piperidin-3-yl)-3-methoxybenzamide (Compound 122)

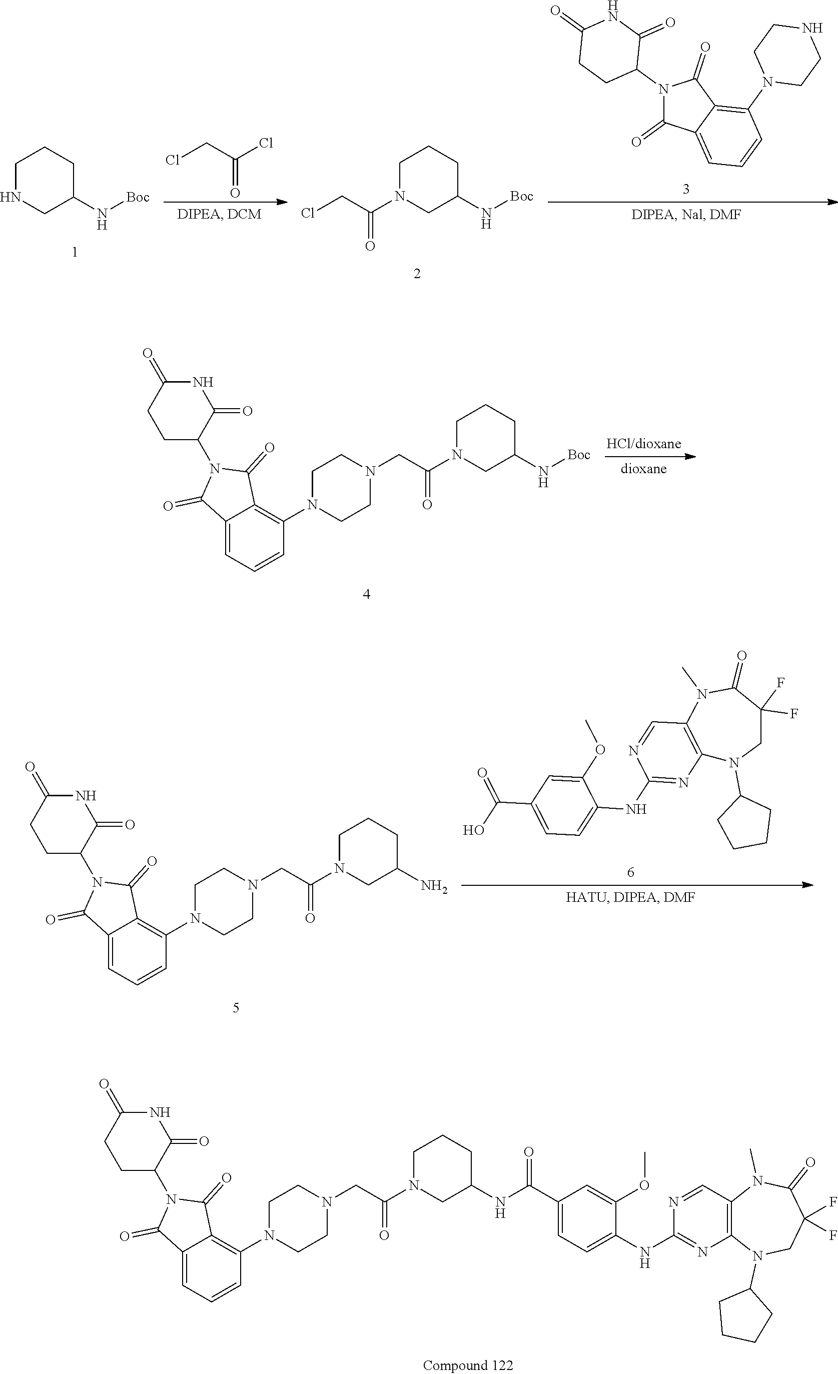

The compound 122 was synthesized by the method described in the scheme similar to the method described in Example 81.

MS(M+H)⁺=912.6, ¹H NMR (400 MHz, DMSO-d₆) δ=10.89-10.79 (m, 1H), 8.30-8.23 (m, 2H), 7.91 (d, J=6.3 Hz, 1H), 7.86 (s, 1H), 7.67 (t, J=7.7 Hz, 1H), 7.52-7.47 (m, 2H), 7.36-7.28 (m, 2H), 5.05 (dd, J=5.4, 12.7 Hz, 1H), 4.81-4.70 (m, 1H), 4.00 (br t, J=14.1 Hz, 4H), 3.95 (s, 3H), 3.40-3.20 (m, 12H), 2.93-2.81 (m, 1H), 2.69-2.62 (m, 6H), 2.09-2.03 (m, 1H), 2.00-1.94 (m, 3H), 1.84-1.77 (m, 1H), 1.75-1.69 (m, 2H), 1.67-1.57 (m, 6H).

### Example 123. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-3-yl)-3-methoxybenzamide (Compound 123)

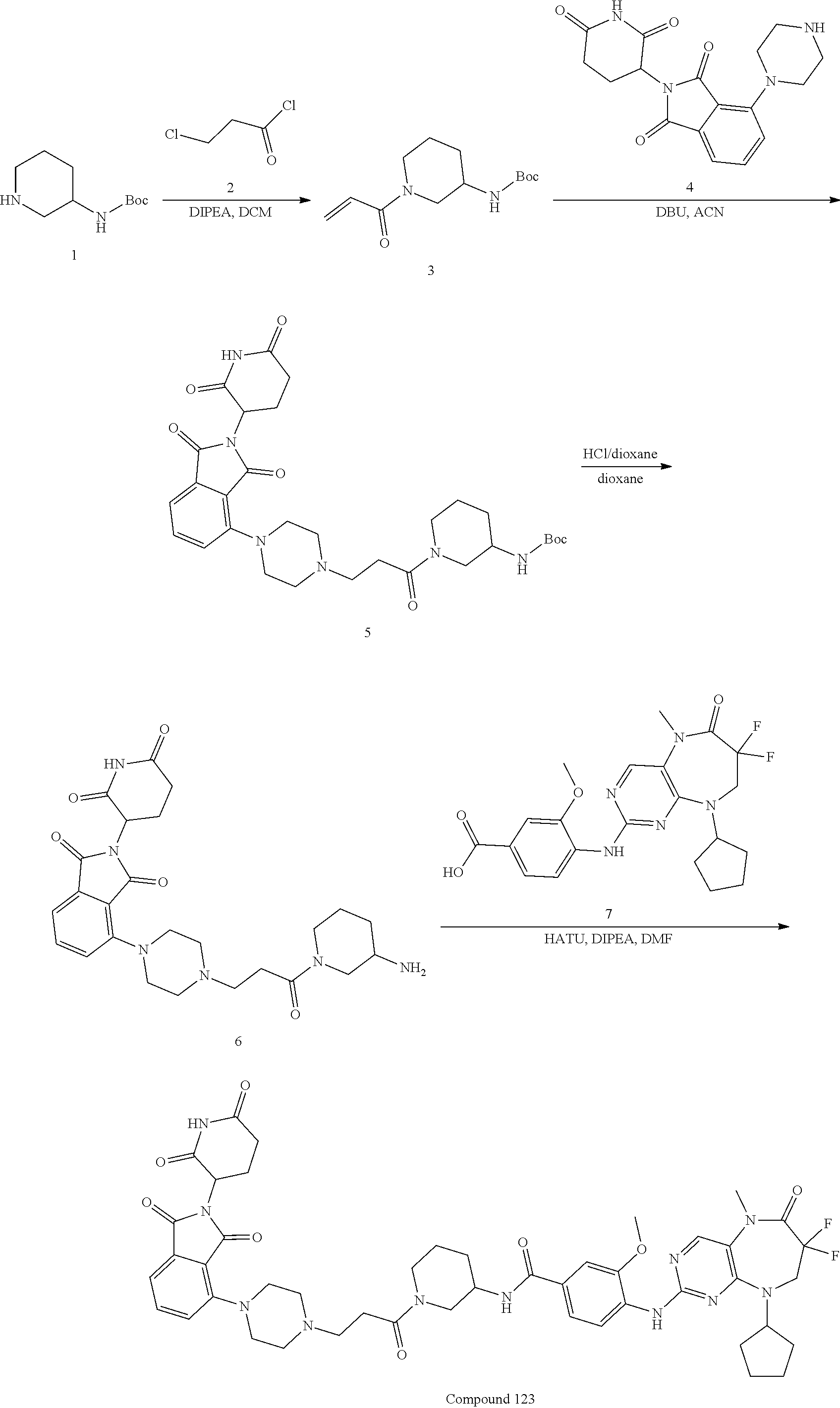

### Step 1. Synthesis of tert-butyl (1-acryloylpiperidin-3-yl)carbamate (3)

To a solution of tert-butyl piperidin-3-ylcarbamate (500 mg, 2.50 mmol) in DCM (5 mL) was added DIPEA (967.98 mg, 7.49 mmol, 1.30 mL), then 3-chloropropanoyl chloride (633.97 mg, 4.99 mmol, 480.28 μL) was added slowly at 0° C. and the mixture was stirred at 0° C. for 0.5 h. LCMS showed a peak (38%) with desired mass. The mixture was concentrated under reduced pressure. The residue was purified by flash silica gel chromatography (5 g SepaFlash® Silica Flash Column, Eluent of 0˜30% EtOAc/Petroleum ether gradient @20 mL/min) to afford tert-butyl (1-acryloylpiperidin-3-yl)carbamate (530 mg, 1.60 mmol, 64.27% yield, 77% purity) as a yellow oil. MS(M+H)⁺=255.1

### Step 2. Synthesis of tert-butyl (1-(3-(4-(2-(2,6-dioxopiperidin-3-yl)-1,3-dioxoisoindolin-4-yl)piperazin-1-yl)propanoyl)piperidin-3-yl)carbamate (5)

To a solution of 2-(2,6-dioxopiperidin-3-yl)-4-(piperazin-1-yl)isoindoline-1,3-dione (480 mg, 1.27 mmol, HCl salt) in ACN (10 mL) was added DBU (447.96 mg, 2.94 mmol, 443.52 μL). Then a solution of tert-butyl (1-acryloylpiperidin-3-yl)carbamate (480 mg, 1.45 mmol, 77% purity) in ACN (5 mL) was added and the mixture was stirred at 20° C. for 14 h. LCMS showed the desired mass. The mixture was diluted with H₂O (10 mL) and EtOAc (10 mL), then extracted with EtOAc (15 mL×3). The combined organic layers was concentrated under reduced pressure. The residue was purified by column chromatography (SiO₂, EtOAc/Methanol=1/0 to 10/1) to afford tert-butyl (1-(3-(4-(2-(2,6-dioxopiperidin-3-yl)-1,3-dioxoisoindolin-4-yl)piperazin-1-yl)propanoyl)piperidin-3-yl)carbamate (160 mg, 260.11 μmol, 26.66% yield, 97% purity) as a yellow solid. MS(M+H)⁺=597.3

### Step 3. Synthesis of 4-(4-(3-(3-aminopiperidin-1-yl)-3-oxopropyl)piperazin-1-yl)-2-(2,6-dioxopiperidin-3-yl)isoindoline-1,3-dione (6)

To a solution of tert-butyl (1-(3-(4-(2-(2,6-dioxopiperidin-3-yl)-1,3-dioxoisoindolin-4-yl)piperazin-1-yl)propanoyl)piperidin-3-yl)carbamate (160 mg, 268.15 μmol) in dioxane (2 mL) was added HCl/dioxane (4 M, 2 mL). The mixture was stirred at 20° C. for 1 h. LCMS showed the desired mass. The mixture was concentrated under reduced pressure to afford 4-(4-(3-(3-aminopiperidin-1-yl)-3-oxopropyl)piperazin-1-yl)-2-(2,6-dioxopiperidin-3-yl)isoindoline-1,3-dione (130 mg, crude, HCl salt) as a yellow solid. MS(M+H)⁺=497.2

### Step 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-3-yl)-3-methoxybenzamide (Compound 123)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (80 mg, 178.80 μmol) in DMF (1 mL) were HATU (101.71 mg, 267.51 μmol) and DIPEA (44.52 mg, 344.47 μmol, 60.00 μL) and the mixture was stirred at 25° C. for 15 min. Then a solution of 4-(4-(3-(3-aminopiperidin-1-yl)-3-oxopropyl)piperazin-1-yl)-2-(2,6-dioxopiperidin-3-yl)isoindoline-1,3-dione (130 mg, 243.89 μmol, HCl salt) and DIPEA (44.52 mg, 344.47 μmol, 60 μL) in DMF (1 mL) was added and the mixture was stirred at 25° C. for 1 h. The mixture was diluted with H₂O (10 mL) and extracted with EtOAc (10 mL×3). The combined organic layer was washed with brine (10 mL×3), dried over Na₂SO₄ and filtered. The filtrate was concentrated under reduced pressure. The crude was purified by prep-HPLC (column: Waters Xbridge 150*25 mm*5 μm; mobile phase: [water(NH₄HCO₃)-ACN]; B %: 39%-69%, 10 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propanoyl)piperidin-3-yl)-3-methoxybenzamide (54 mg, 54.23 μmol, 30.33% yield, 93% purity) as a yellow solid. MS(M+H)⁺=926.1.

¹H NMR (400 MHz, DMSO-d₆) δ=10.82 (br s, 1H), 8.28 (d, J=8.3 Hz, 1H), 8.25 (s, 1H), 7.98-7.89 (m, 1H), 7.86 (s, 1H), 7.66 (t, J=7.8 Hz, 1H), 7.54-7.46 (m, 2H), 7.34-7.27 (m, 2H), 5.05 (dd, J=5.6, 12.6 Hz, 1H), 4.81-4.71 (m, 1H), 4.05-3.94 (m, 6H), 3.89-3.78 (m, 1H), 3.34 (s, 3H), 3.33-3.28 (m, 4H), 2.92-2.82 (m, 1H), 2.71-2.66 (m, 2H), 2.65-2.52 (m, 9H), 2.11-1.90 (m, 5H), 1.85-1.35 (m, 10H).

### Example 124. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-((4-(2-(2,6-dioxopiperidin-3-yl)-1,3-dioxoisoindolin-4-yl)piperazin-1-yl)methyl)piperidin-1-yl)-3-methoxybenzamide (Compound 124)

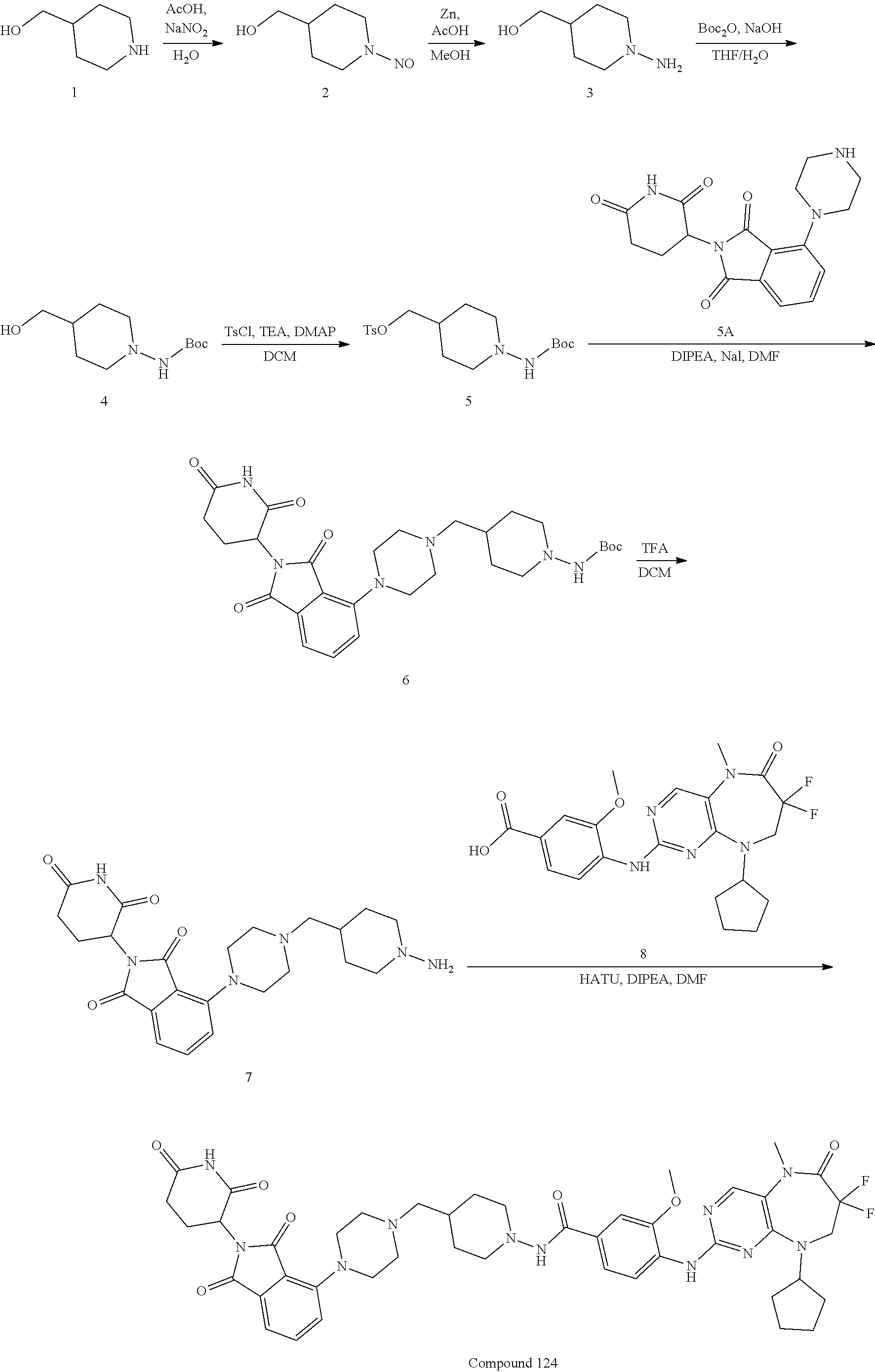

### Step 1. Synthesis of (1-nitrosopiperidin-4-yl)methanol (2)

To a solution of 4-piperidylmethanol (5 g, 43.41 mmol) in H₂O (40 mL) was added NaNO₂ (5.99 g, 86.83 mmol) in portions at 0° C. Then AcOH (7.82 g, 130.24 mmol) was added drop-wise at 0° C. The resulting mixture was allowed to warm to 25° C. slowly and stirred for 4 hrs. TLC (EtOAc:methanol=10:1, R_{f}=0.4) showed a big new spot with strong absorption under 254 nm UV was formed. LCMS showed a main peak with desired mass. The reaction solution was adjust pH to 8 with NaHCO₃ solution (about 150 mL). The resulting mixture was extracted with EtOAc/methanol (10:1, 50 mL×10) (Note: The product has good solubility in water which makes the extraction difficult.). The combined organic layers were dried over Na₂SO₄ and concentrated to afford (1-nitrosopiperidin-4-yl)methanol (6.5 g, crude) as yellow oil. MS(M+H)⁺=145.2.

### Step 2. Synthesis of (1-aminopiperidin-4-yl)methanol (3)

To a solution of (1-nitrosopiperidin-4-yl)methanol (6.5 g, 45.09 mmol) in MeOH (60 mL) was added Zn (15.57 g, 238.11 mmol) at 0° C. followed by the addition of AcOH (40.61 g, 676.28 mmol) drop-wise at 0° C. The resulting mixture was allowed to warm to 25° C. slowly and stirred for 1 h. TLC (EtOAc/methanol=10:1, R_{f}=0) showed the starting material was consumed completely. LCMS showed the starting material was consumed completely and the desired mass. The reaction mixture was filtered through celite pad and washed with MeOH (200 mL). The filtrate was concentrated to afford a yellow solid. The yellow solid was treated with MeOH (80 mL) and stirred for 30 mins and white solid formed (Note: the white solid was inorganic salts). The mixture was filtered and the filtrate was concentrated to afford (1-aminopiperidin-4-yl)methanol (16 g, crude) as yellow oil. The crude product was used for the next step directly. MS(M+H)⁺=131.2.

¹H NMR (400 MHz, Methanol-d₄) δ=3.43 (d, J=6.2 Hz, 2H), 3.42-3.37 (m, 2H), 2.78 (br t, J=11.4 Hz, 2H), 1.91 (br d, J=14.2 Hz, 2H), 1.68-1.58 (m, 1H), 1.52-1.41 (m, 2H)

### Step 3. Synthesis of tert-butyl (4-(hydroxymethyl)piperidin-1-yl)carbamate (4)

To a solution of (1-aminopiperidin-4-yl)methanol (14 g, 107.54 mmol) in THF (100 mL) was added a solution of NaOH (12.90 g, 322.61 mmol) in H₂O (60 mL) at 25° C. followed by the addition of Boc₂O (23.47 g, 107.54 mmol, 24.71 mL). The resulting mixture was stirred at 25° C. for 16 hrs. LCMS showed the desired mass. The reaction mixture was filtered. LCMS showed no product in the filter cake. Water (100 mL) was added to the filtrate and the mixture was extracted with EtOAc (60 mL×4). The combined organic layers were dried over Na₂SO₄ and concentrated to give the crude product. The crude product was treated with petroleum ether (50 mL) and stirred for 30 mins. The mixture was filtered. The filter cake was collected and dried to afford a yellow solid which was treated with petroleum ether/EtOAc (30 mL, 10:1) for 30 mins. The mixture was filtered. The filter cake was collected and dried to afford tert-butyl (4-(hydroxymethyl)piperidin-1-yl)carbamate (4 g, 17.37 mmol, 16.15% yield) as a white solid. MS(M+Na)⁺=253.1.

¹H NMR (400 MHz, DMSO-d₆) δ=7.87 (s, 1H), 4.43 (s, 1H), 3.23-3.19 (m, 2H), 2.86-2.81 (m, 2H), 2.45-2.40 (m, 2H), 1.63-1.58 (m, 2H), 1.36 (s, 9H), 1.26-1.21 (m, 1H), 1.19-1.10 (m, 2H).

### Step 4. Synthesis of (1-((tert-butoxycarbonyl)amino)piperidin-4-yl)methyl 4-methylbenzenesulfonate (5)

To a solution of tert-butyl (4-(hydroxymethyl)piperidin-1-yl)carbamate (1 g, 4.34 mmol) and TsCl (1.24 g, 6.51 mmol) in DCM (20 mL) were added TEA (1.32 g, 13.03 mmol) and DMAP (53.05 mg, 434.21 μmol) at 25° C. The resulting mixture was stirred at 25° C. for 16 hrs. LCMS showed the starting material was consumed completely and the desired mass. The reaction solution was concentrated to give the crude product. The crude product was purified by flash silica gel chromatography (20 g silca gel column, EtOAc/petroleum ether=10-35%, 80 mL/min) to afford (1-((tert-butoxycarbonyl)amino)piperidin-4-yl)methyl 4-methylbenzenesulfonate (500 mg, 1.22 mmol, 28.15% yield, 94% purity) as a white solid. MS(M+H)⁺=384.9.

¹H NMR (400 MHz, CDCl₃) δ=7.77 (d, J=8.0 Hz, 2H), 7.35 (d, J=8.0 Hz, 2H), 5.42-5.40 (m, 1H), 3.83 (d, J=8.0 Hz, 2H), 3.15-3.07 (m, 2H), 2.46 (s, 3H), 2.38-2.32 (m, 2H), 1.71-1.67 (m, 2H), 1.46-1.45 (m, 1H), 1.44 (s, 9H), 1.41-1.35 (m, 2H).

### Step 5. Synthesis of tert-butyl (4-((4-(2-(2,6-dioxopiperidin-3-yl)-1,3-dioxoisoindolin-4-yl)piperazin-1-yl)methyl) piperidin-1-yl)carbamate (6)

To a solution of (1-((tert-butoxycarbonyl)amino)piperidin-4-yl)methyl 4-methylbenzenesulfonate (350 mg, 910.30 μmol) and 2-(2,6-dioxopiperidin-3-yl)-4-(piperazin-1-yl)isoindoline-1,3-dione (378.02 mg, 910.30 μmol, 2HCl) in DMF (6 mL) were added DIPEA (470.60 mg, 3.64 mmol) and NaI (27.29 mg, 182.06 μmol) at 25° C. The resulting mixture was stirred at 60° C. for 14 hrs. LCMS showed the starting material was consumed completely and the desired mass. The reaction mixture was poured into brine (20 mL). The mixture was extracted with EtOAc (10 mL×4). The combined organic layers were washed with brine (10 mL×2), dried over Na₂SO₄ and concentrated to give the crude product. The crude product was purified by flash silica gel chromatography (10 g silica gel column, EtOAc/petroleum ether=20-70%, 50 mL/min) to afford tert-butyl (4-((4-(2-(2,6-dioxopiperidin-3-yl)-1,3-dioxoisoindolin-4-yl)piperazin-1-yl)methyl)piperidin-1-yl)carbamate (80 mg, crude) as yellow solid and tert-butyl (4-((4-(2-(2,6-dioxopiperidin-3-yl)-1,3-dioxoisoindolin-4-yl)piperazin-1-yl)methyl)piperidin-1-yl)carbamate (180 mg, crude) as a yellow solid. MS(M+H)⁺=555.3.

### Step 6. Synthesis of 4-(4-((1-aminopiperidin-4-yl)methyl)piperazin-1-yl)-2-(2,6-dioxopiperidin-3-yl)isoindoline-1,3-dione (7)

A solution of tert-butyl (4-((4-(2-(2,6-dioxopiperidin-3-yl)-1,3-dioxoisoindolin-4-yl)piperazin-1-yl)methyl)piperidin-1-yl)carbamate (90 mg, 162.27 μmol) and TFA (770.00 mg, 6.75 mmol, 0.5 mL) in DCM (2 mL) was stirred at 25° C. for 1 h. LCMS showed the starting material consumed completely and the desired mass. The reaction solution was concentrated to afford 4-(4-((1-aminopiperidin-4-yl)methyl)piperazin-1-yl)-2-(2,6-dioxopiperidin-3-yl)isoindoline-1,3-dione (90 mg, crude, TFA) as yellow oil. The crude product was used for the next step directly. MS(M+H)⁺=455.3.

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-((4-(2-(2,6-dioxopiperidin-3-yl)-1,3-dioxoisoindolin-4-yl)piperazin-1-yl)methyl)piperidin-1-yl)-3-methoxybenzamide (Compound 124)

To a solution of 4-(4-((1-aminopiperidin-4-yl)methyl)piperazin-1-yl)-2-(2,6-dioxopiperidin-3-yl)isoindoline-1,3-dione (90 mg, crude, TFA), 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (63.75 mg, 142.47 μmol) and DIPEA (122.75 mg, 949.79 μmol) in DMF (2 mL) was added HATU (78.25 mg, 205.79 μmol) at 25° C. The resulting mixture was stirred at 25° C. for 2 hrs. LCMS showed a main peak with desired mass. The reaction solution was poured into brine (12 mL). The resulting mixture was extracted with EtOAc (5 mL×4). The combined organic layers were dried over Na₂SO₄ and concentrated to afford the crude product. The crude product was purified by flash silica gel chromatography (4 g silica gel column, EtOAc/petroleum ether=20-100% and then MeOH/EtOAc=20%, 40 mL/min), followed by prep-HPLC (column: Waters Xbridge 150*25 mm*5 μm; mobile phase: [water(NH₄HCO₃)-ACN]; B %: 44%-64%, 10 min) and lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-((4-(2-(2,6-dioxopiperidin-3-yl)-1,3-dioxoisoindolin-4-yl)piperazin-1-yl)methyl)piperidin-1-yl)-3-methoxybenzamide (10.6 mg, 11.30 μmol, 7.14% yield, 94.2% purity) as a yellow solid. MS(M+H)⁺=884.4.

¹H NMR (400 MHz, CDCl₃) δ=8.47 (d, J=8.4 Hz, 1H), 8.41-8.17 (m, 1H), 8.06 (s, 1H), 7.76 (s, 1H), 7.64-7.56 (m, 1H), 7.41 (d, J=6.7 Hz, 2H), 7.23 (s, 1H), 7.18 (d, J=8.4 Hz, 1H), 7.04-6.81 (m, 1H), 4.97 (dd, J=5.4, 12.3 Hz, 1H), 4.82 (q, J=8.4 Hz, 1H), 3.98 (s, 3H), 3.90 (t, J=13.4 Hz, 2H), 3.41 (s, 3H), 3.38-3.34 (m, 4H), 3.32-3.34 (m, 1H), 3.07-2.96 (m, 1H), 2.94-2.81 (m, 2H), 2.79-2.57 (m, 7H), 2.50-2.38 (m, 2H), 2.37-2.28 (m, 1H), 2.20-2.00 (m, 5H), 1.74-1.66 (m, 4H), 1.64-1.54 (m, 4H).

### Example 125. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(((1-(2-(2,6-dioxopiperidin-3-yl)-1,3-dioxoisoindolin-4-yl)piperidin-4-yl)oxy)methyl)phenyl)-2-fluoro-5-methoxybenzamide (Compound 125)

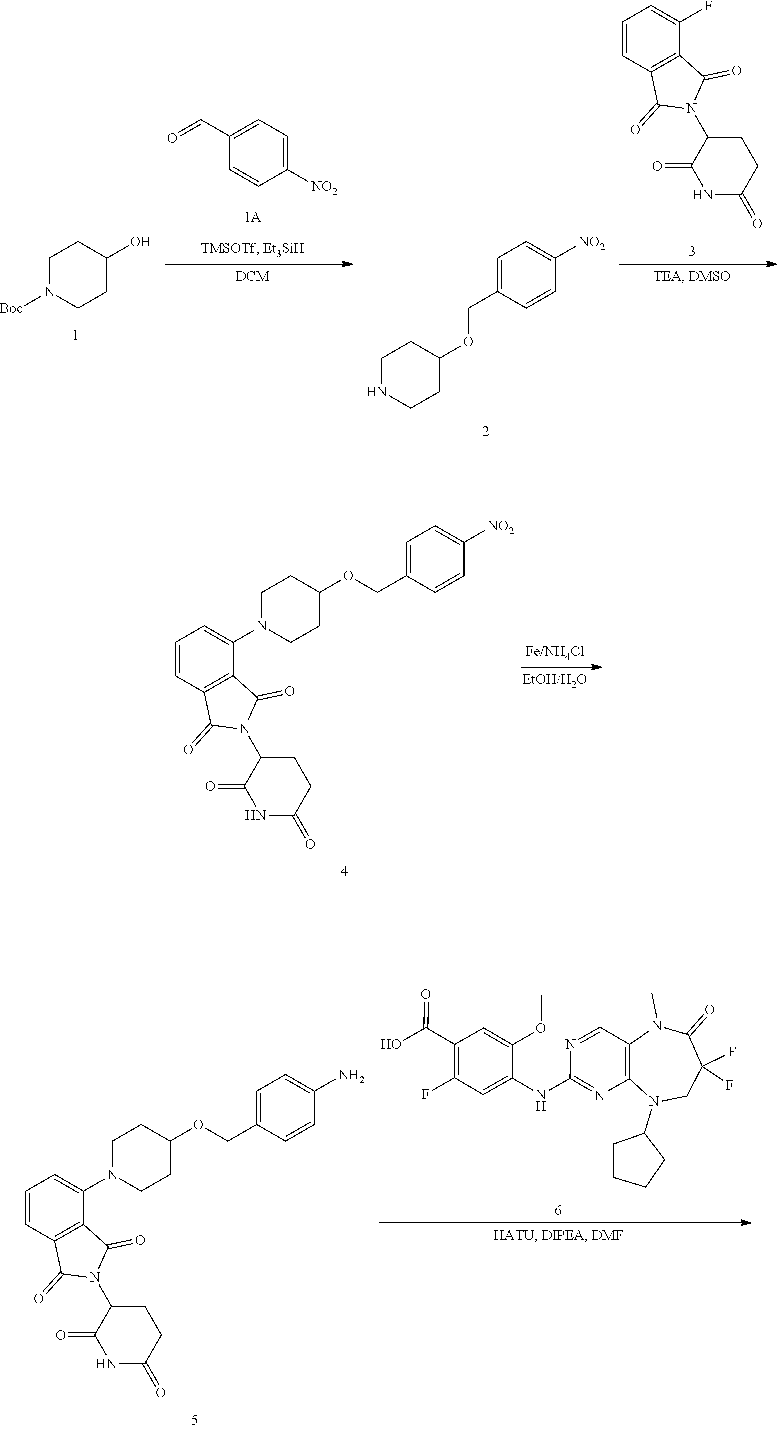

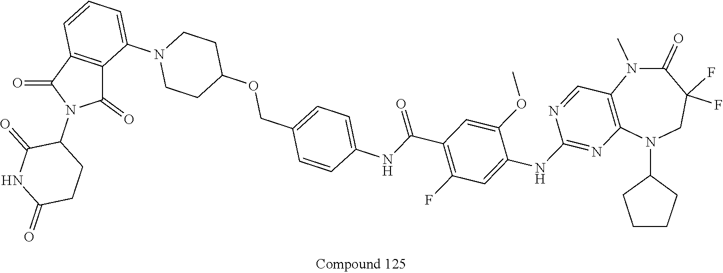 [SMILES: COc1cc(C(=O)Nc2ccc(COC3CCN(c4cccc5c4C(=O)N(C4CCC(=O)NC4=O)C5=O)CC3)cc2)c(F)cc1Nc1ncc2c(n1)N(C1CCCC1)CC(F)(F)C(=O)N2C]

### Step 1. Synthesis of 4-((4-nitrobenzyl)oxy)piperidine (2)

To a solution of tert-butyl 4-hydroxypiperidine-1-carboxylate (2 g, 9.94 mmol) and 4-nitrobenzaldehyde (1.50 g, 9.94 mmol) in DCM (20 mL) was added TESiH (3.47 g, 29.81 mmol, 4.76 mL) at 0° C. Then TMSOTf (4.42 g, 19.87 mmol, 3.59 mL) was added to the mixture at 0° C. after 1 h. The mixture was stirred at 25° C. for 1 h. LCMS showed 35% of desired mass was detected. The mixture solution was concentrated under reduced pressure to afford 4-((4-nitrobenzyl)oxy)piperidine (8 g, crude) as yellow oil, which was used for the next step directly. MS(M+H)⁺=237.1

### Step 2. Synthesis of 2-(2,6-dioxopiperidin-3-yl)-4-(4-((4-nitrobenzyl)oxy)piperidin-1-yl)isoindoline-1,3-dione (4)

To a solution of 4-((4-nitrobenzyl)oxy)piperidine (8 g, crude) in DMSO (20 mL) were added TEA (3.60 g, 35.55 mmol, 4.95 mL) and 2-(2,6-dioxopiperidin-3-yl)-4-fluoroisoindoline-1,3-dione (3.27 g, 11.85 mmol) at 25° C. The mixture was stirred at 100° C. for 12 h. LCMS showed ˜35% of desired mass was detected. The reaction mixture was diluted with water (100 mL) and extracted with EtOAc (100 mL×3). The combined organic layers were washed with saturated brine (150 mL), dried over anhydrous sodium sulfate, filtered and concentrated under reduced pressure to give a residue. The residue was purified by flash silica gel chromatography (ISCO®; 80 g SepaFlash® Silica Flash Column, Eluent of 0˜70% petroleum ether:EtOAc gradient @80 mL/min) to afford 2-(2,6-dioxopiperidin-3-yl)-4-(4-((4-nitrobenzyl)oxy)piperidin-1-yl)isoindoline-1,3-dione (1.82 g, 3.57 mmol, 30.12% yield, 96.6% purity) as a yellow solid. MS(M+H)⁺=493.1

¹H NMR (400 MHz, DMSO-d₆) δ=11.08 (s, 1H), 8.22 (d, J=8.4 Hz, 2H), 7.70-7.66 (m, 1H), 7.65-7.63 (m, 2H), 7.34 (t, J=8.4 Hz, 2H), 5.11-5.07 (m, 1H), 4.72 (s, 2H), 3.69-3.66 (m, 1H), 3.57-3.53 (m, 2H), 3.13-3.08 (m, 2H), 2.88-2.82 (m, 1H), 2.63-2.60 (m, 1H), 2.04-1.98 (m, 4H), 1.79-1.72 (m, 2H).

### Step 3. Synthesis of 4-(4-((4-aminobenzyl)oxy)piperidin-1-yl)-2-(2,6-dioxopiperidin-3-yl)isoindoline-1,3-dione (5)

To a solution of 2-(2,6-dioxopiperidin-3-yl)-4-(4-((4-nitrobenzyl)oxy)piperidin-1-yl)isoindoline-1,3-dione (0.3 g, 609.16 μmol) in H₂O (5 mL) and EtOH (10 mL) were added Fe (204.11 mg, 3.65 mmol) and NH₄Cl (195.51 mg, 3.65 mmol) at 25° C. The mixture was stirred at 25° C. for 2 h. LCMS showed a main peak with desired mass was detected. The reaction mixture was diluted with water (30 mL) and extracted with EtOAc (50 mL×5). The combined organic layers were washed with saturated brine (50 mL), dried over anhydrous sodium sulfate, filtered and concentrated under reduced pressure to afford 4-(4-((4-aminobenzyl)oxy)piperidin-1-yl)-2-(2,6-dioxopiperidin-3-yl)isoindoline-1,3-dione (0.2 g, 327.35 μmol, 53.74% yield, 75.7% purity) as yellow oil, which was used for the next step directly. MS(M+H)⁺=463.2

### Step 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(((1-(2-(2,6-dioxopiperidin-3-yl)-1,3-dioxoisoindolin-4-yl)piperidin-4-yl)oxy)methyl)phenyl)-2-fluoro-5-methoxybenzamide (Compound 125)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (80 mg, 171.89 μmol) in DMF (2 mL) were added HATU (130.71 mg, 343.77 μmol) and DIPEA (66.65 mg, 515.66 μmol, 89.82 μL) at 25° C. Then 4-(4-((4-aminobenzyl)oxy)piperidin-1-yl)-2-(2,6-dioxopiperidin-3-yl)isoindoline-1,3-dione (105.02 mg, 171.89 μmol, 75.7% purity) was added to the mixture 0.5 h later. The mixture was stirred at 25° C. for 1 h. LCMS showed ˜27% of desired mass was detected. The reaction mixture was diluted with water (30 mL) and extracted with EtOAc (50 mL×3). The combined organic layers were washed with saturated brine (50 mL), dried over anhydrous sodium sulfate, filtered and concentrated under reduced pressure to give a residue. The residue was purified by flash silica gel chromatography (ISCO®; 20 g SepaFlash® Silica Flash Column, Eluent of 0˜80% petroleum ether:EtOAc gradient @60 mL/min) and then prep-HPLC (column: Waters Xbridge 150×25 mm×5 μm; mobile phase: [water(NH₄HCO₃)-ACN]; B %: 58%-88%, 10 min) followed by lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(((1-(2-(2,6-dioxopiperidin-3-yl)-1,3-dioxoisoindolin-4-yl)piperidin-4-yl)oxy)methyl)phenyl)-2-fluoro-5-methoxybenzamide (57 mg, 58.45 μmol, 34.00% yield, 93.3% purity) as yellow solid. MS (M+H)⁺=910.4.

¹H NMR (400 MHz, DMSO-d₆) δ=11.08 (br s, 1H), 10.15 (s, 1H), 8.32-8.29 (m, 2H), 8.10 (s, 1H), 7.70-7.65 (m, 3H), 7.36-7.32 (m, 4H), 7.27 (d, J=6.8 Hz, 1H), 5.11-5.06 (m, 1H), 4.85-4.81 (m, 1H), 4.52 (s, 2H), 4.07 (t, J=14 Hz, 2H), 3.94 (s, 3H), 3.62-3.60 (m, 1H), 3.55-3.52 (m, 2H), 3.33 (s, 3H), 3.11-3.06 (m, 2H), 2.90-2.82 (m, 1H), 2.60-2.53 (m, 2H), 2.03-1.96 (m, 5H), 1.72-1.61 (m, 8H).

### Example 126. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-((1-(2-(2,6-dioxopiperidin-3-yl)-1,3-dioxoisoindolin-4-yl)piperidin-4-yl)methyl)piperidin-3-yl)-3-methoxybenzamide (Compound 126)

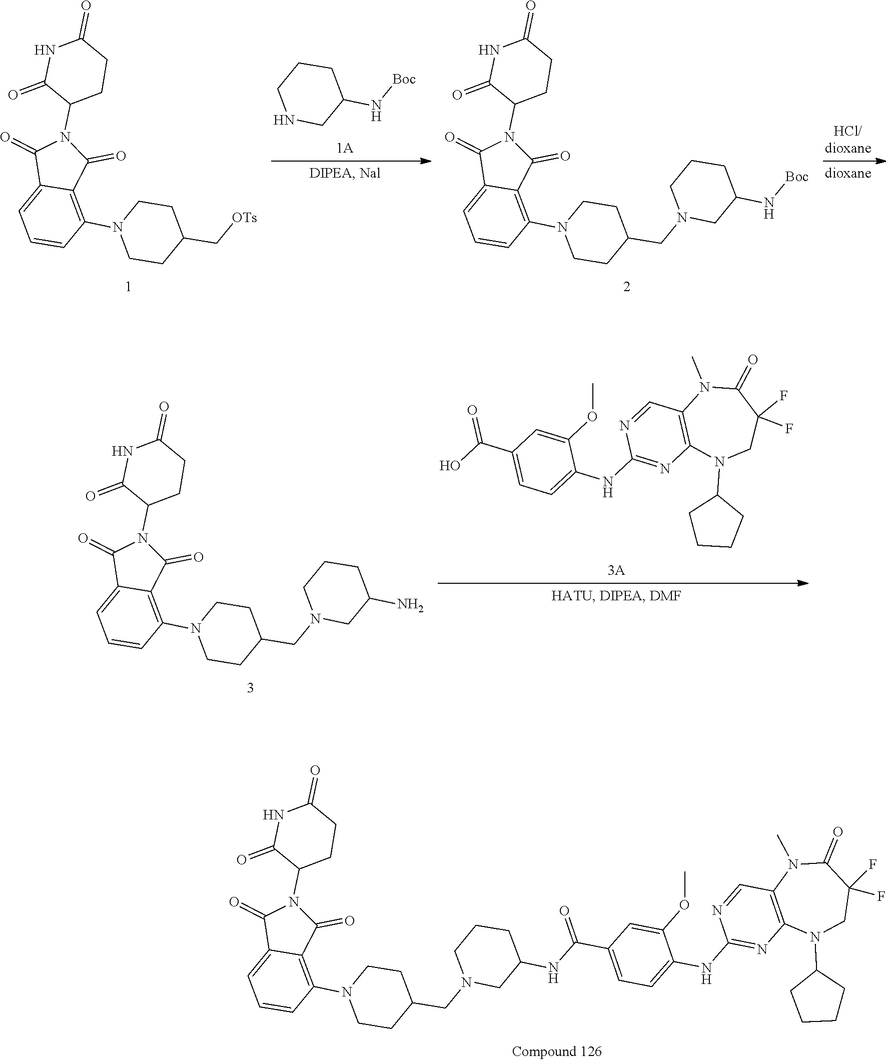 [SMILES: Cc1ccc(S(=O)(=O)OCC2CCN(c3cccc4c3C(=O)N(C3CCC(=O)NC3=O)C4=O)CC2)cc1 ; CC(C)(C)OC(=O)NC1CCCNC1 ; CC(C)(C)OC(=O)NC1CCCN(CC2CCN(c3cccc4c3C(=O)N(C3CCC(=O)NC3=O)C4=O)CC2)C1 ; NC1CCCN(CC2CCN(c3cccc4c3C(=O)N(C3CCC(=O)NC3=O)C4=O)CC2)C1 ; COc1cc(C(=O)O)ccc1Nc1ncc2c(n1)N(C1CCCC1)CC(F)(F)C(=O)N2C ; COc1cc(C(=O)NC2CCCN(CC3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C]

The compound 126 was synthesized by the method described in the scheme similar to the method described in Example 60.

MS(M+H)⁺=883.4, ¹H NMR (400 MHz, DMSO-d₆) δ=11.05 (s, 1H), 8.31-8.21 (m, 2H), 8.07-7.99 (m, 1H), 7.95 (s, 1H), 7.60-7.42 (m, 3H), 7.14-7.05 (m, 2H), 5.11-5.01 (m, 1H), 4.83-4.70 (m, 1H), 4.10-3.99 (m, 2H), 3.98-3.87 (m, 4H), 3.70-3.53 (m, 2H), 3.53-3.45 (m, 1H), 3.32 (s, 3H), 2.97-2.72 (m, 3H), 2.62-2.52 (m, 3H), 2.42-2.34 (m, 2H), 2.28-2.18 (m, 1H), 2.15-2.08 (m, 1H), 2.02-1.79 (m, 6H), 1.75-1.66 (m, 3H), 1.65-1.46 (m, 8H), 1.44-1.29 (m, 1H).

### Example 127. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(2-(2,6-dioxopiperidin-3-yl)-1,3-dioxoisoindolin-5-yl)piperazin-1-yl)acetyl)piperidin-4-yl)-3-methoxybenzamide (Compound 127)

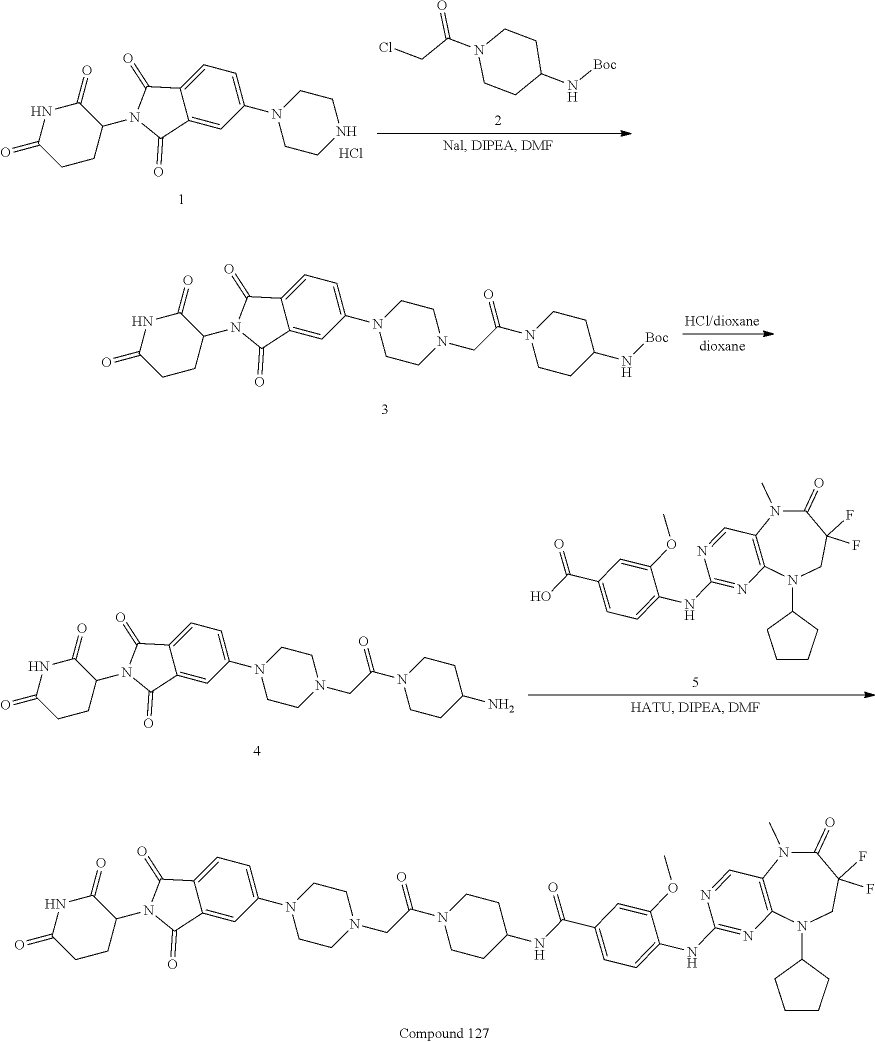

The compound 127 was synthesized by the method described in the scheme similar to the method described in Example 60.

MS(M+H)⁺=912.5, ¹H NMR (400 MHz, DMSO-d₆) δ=11.08 (s, 1H), 8.23 (s, 1H), 8.15-8.13 (m, 1H), 8.00 (s, 1H), 7.74 (d, J=8.0 Hz, 1H), 7.68 (d, J=8.0 Hz, 1H), 7.34 (d, J=2.0 Hz, 1H), 7.27-7.24 (m, 1H), 7.02 (d, J=1.6 Hz, 1H), 6.96-6.94 (m, 1H), 5.09-5.04 (m, 1H), 4.72-4.68 (m, 1H), 4.05-3.85 (m, 7H), 3.49-3.47 (m, 4H), 3.31-3.30 (m, 7H), 2.99 (s, 2H), 2.91-2.83 (m, 1H), 2.61-2.56 (m, 5H), 2.03-1.97 (m, 1H), 1.92-1.85 (m, 2H), 1.77-1.62 (m, 4H), 1.60-1.44 (in, 6H).

### Example 128. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1s,4s)-4-((2-(4-(2-(2,6-dioxopiperidin-3-yl)-1,3-dioxoisoindolin-4-yl)piperazin-1-yl)ethyl)(methyl)amino)cyclohexyl)-3-methoxybenzamide (cis) (Compound 128)

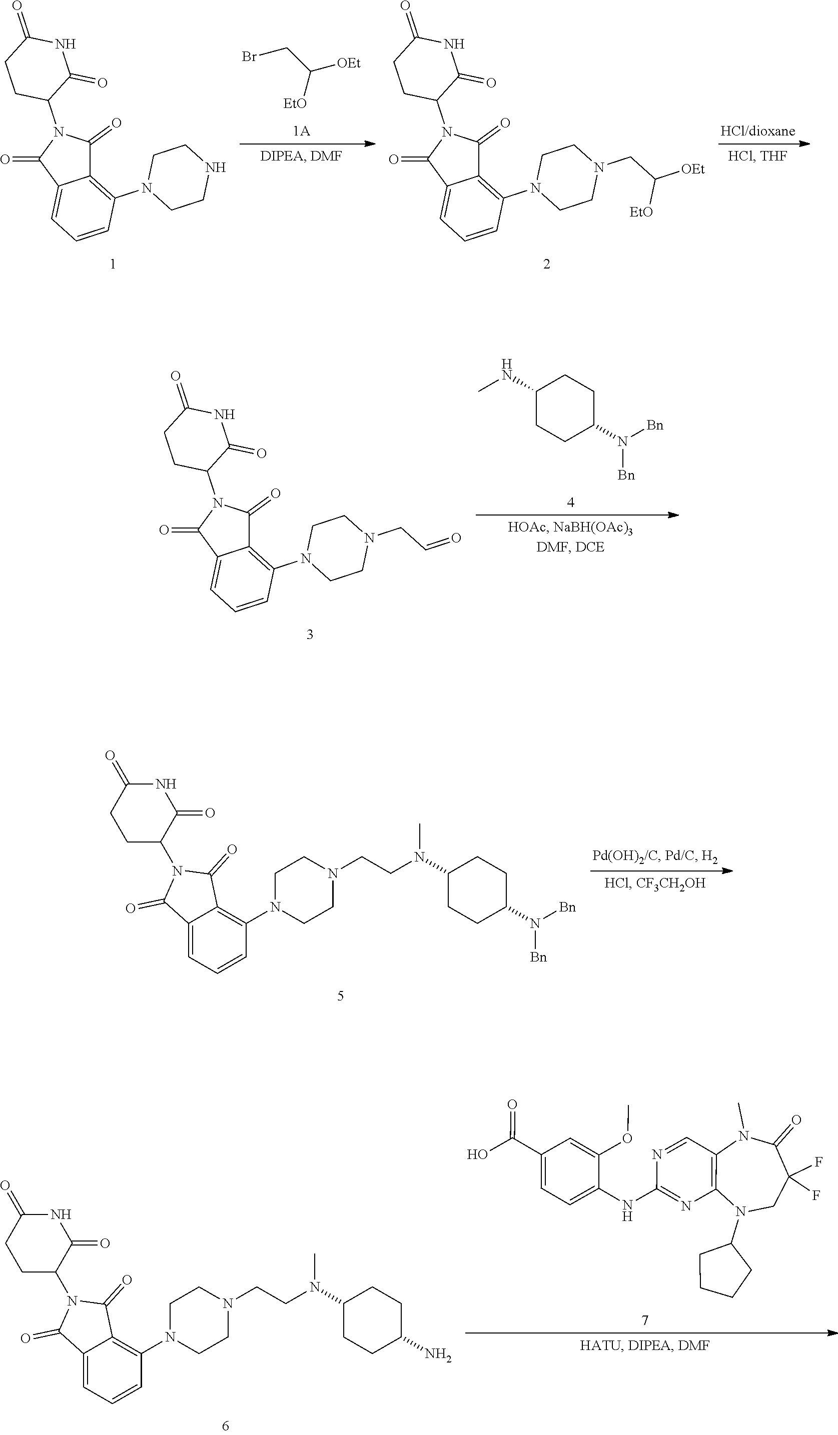

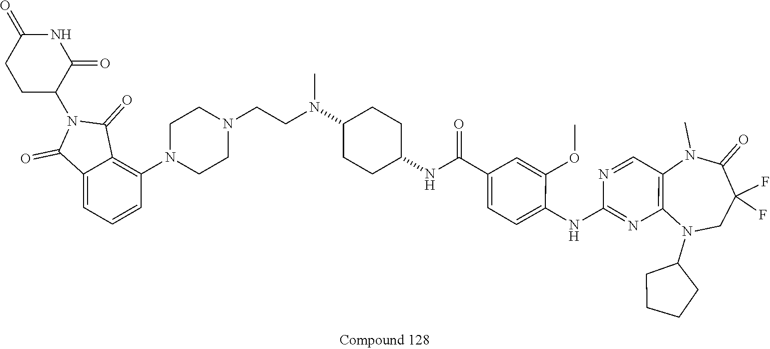

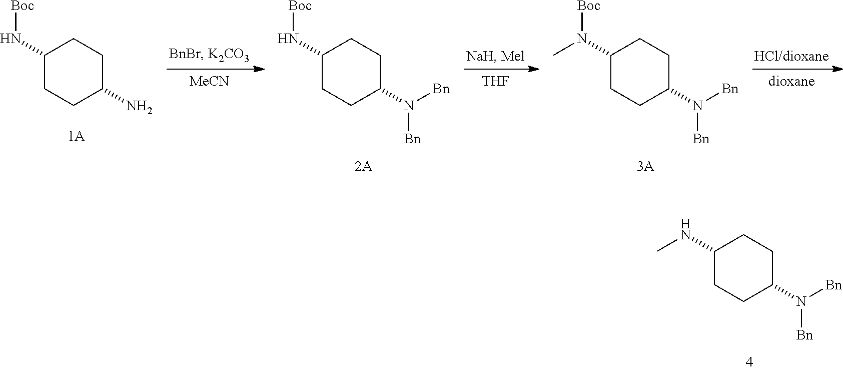 [SMILES: "MeCN" ; CC(C)(C)OC(=O)N[C@H]1CC[C@@H](N)CC1 ; CC(C)(C)OC(=O)N[C@H]1CC[C@@H](N(Cc2ccccc2)Cc2ccccc2)CC1 ; CN(C(=O)OC(C)(C)C)[C@H]1CC[C@@H](N(Cc2ccccc2)Cc2ccccc2)CC1 ; "THF" ; CN[C@H]1CC[C@@H](N(Cc2ccccc2)Cc2ccccc2)CC1]

### Step 1. Synthesis of tert-butyl ((1s,4s)-4-(dibenzylamino)cyclohexyl)carbamate (cis) (2A)

A mixture of tert-butyl ((1s,4s)-4-aminocyclohexyl)carbamate (cis) (600.00 mg, 2.80 mmol), (bromomethyl)benzene (1.20 g, 7.00 mmol, 831.35 μL) and K₂CO₃ (1.55 g, 11.20 mmol) in ACN (30 mL) was stirred at 60° C. for 16 hours. LCMS showed tert-butyl ((1s,4s)-4-aminocyclohexyl)carbamate (cis) was consumed and a peak (41%) with desired mass. The mixture was filtered and the filter cake was washed with EtOAc (50 mL) and ACN (30 mL), the filtrate was concentrated in vacuum to afford tert-butyl ((1s,4s)-4-(dibenzylamino)cyclohexyl)carbamate (cis) (1.1 g, crude) as a white solid which was used to next step directly. MS(M+H)⁺=395.2

### Step 2. Synthesis of tert-butyl ((1s,4s)-4-(dibenzylamino)cyclohexyl)(methyl)carbamate (cis) (3A)

To a solution of tert-butyl ((1s,4s)-4-(dibenzylamino)cyclohexyl)carbamate (cis) (1 g, 2.53 mmol) in THF (25 mL) was added NaH (253.45 mg, 6.34 mmol, 60% purity) at 5° C., the suspension was stirred at 5° C. for 30 minutes. To the mixture was added iodomethane (467.67 mg, 3.29 mmol, 205.12 μL, 1.3 eq), the mixture was stirred at 25° C. for 1.5 hours. TLC (SiO2, Petroleum ether:EtOAc=10:1) indicated the starting material was consumed completely and one major new spot with lower polarity was detected. The mixture was poured into ice water (60 mL) at 0° C., and then extracted with EtOAc (60 mL×3). The combined organic layers were washed with brine (100 mL×2), dried over Na₂SO₄, filtered and concentrated in vacuum. The residue was purified by flash silica gel chromatography (12 g SepaFlash® Silica Flash Column, Eluent of 2˜3% EtOAc/Petroleum ether gradient @100 mL/min), the eluent was concentrated in vacuum to afford tert-butyl ((1s,4s)-4-(dibenzylamino)cyclohexyl)(methyl)carbamate (cis) (820 mg, 2.01 mmol, 79.18% yield) as a colorless oil. MS(M+H)⁺=409.6

### Step 3. Synthesis of (1s,4s)-N1,N1-dibenzyl-N4-methylcyclohexane-1,4-diamine (cis) (4)

To a solution of tert-butyl ((1s,4s)-4-(dibenzylamino)cyclohexyl)(methyl)carbamate (cis) (820.00 mg, 2.01 mmol) in dioxane (5 mL) was added HCl/dioxane (4 M, 10 mL), the mixture was stirred at 25° C. for 2 hours. TLC (SiO2, Petroleum ether:EtOAc=20:1) indicated the starting material was consumed completely and one major new spot with larger polarity was detected. The reaction mixture was concentrated in vacuum. The residue was diluted with H₂O (30 mL), to the mixture was added saturated NaHCO₃ solution to adjust pH>7, the resulting mixture was extracted with EtOAc (30 mL×3). The combined organic layers were dried over Na₂SO₄, filtered and concentrated in vacuum to afford (1s,4s)-N1,N1-dibenzyl-N4-methylcyclohexane-1,4-diamine (cis) (545 mg, crude) as a brown solid. MS(M+H)⁺=309.2

### Step 4. Synthesis of 4-(4-(2,2-diethoxyethyl)piperazin-1-yl)-2-(2,6-dioxopiperidin-3-yl)isoindoline-1,3-dione (2)

To a solution of 2-(2,6-dioxopiperidin-3-yl)-4-(piperazin-1-yl)isoindoline-1,3-dione (600 mg, 1.58 mmol, HCl salt) and 2-bromo-1,1-diethoxyethane (936.42 mg, 4.75 mmol, 714.83 μL) in DMF (12 mL) was added DIPEA (1.02 g, 7.92 mmol, 1.38 mL), the mixture was stirred at 80° C. for 16 hours. LCMS showed 2-(2,6-dioxopiperidin-3-yl)-4-(piperazin-1-yl)isoindoline-1,3-dione was consumed completely and a peak (77%) with desired mass. The mixture was combined with another batch (200 mg). The resulting mixture was diluted with H₂O (100 mL) and extracted with EtOAc (60 mL×3). The combined organic layers were washed with brine (100 mL×5), dried over Na₂SO₄, filtered and concentrated in vacuum. The residue was triturated with a mixture of MTBE (15 mL) and EtOAc (5 mL) for 10 minutes, the suspension was filtered and the filter cake was washed with MTBE (10 mL), the filter cake was collected and dried to afford 4-(4-(2,2-diethoxyethyl)piperazin-1-yl)-2-(2,6-dioxopiperidin-3-yl)isoindoline-1,3-dione (679 mg, 1.48 mmol, 93.50% yield) as a yellow solid. MS(M+H)⁺=459.1

### Step 5. Synthesis of 2-(4-(2-(2,6-dioxopiperidin-3-yl)-1,3-dioxoisoindolin-4-yl)piperazin-1-yl)acetaldehyde (3)

To a solution of 4-(4-(2,2-diethoxyethyl)piperazin-1-yl)-2-(2,6-dioxopiperidin-3-yl)isoindoline-1,3-dione (620 mg, 1.35 mmol) in THF (5 mL) was added HCl (1 M, 0.35 mL) and HCl/dioxane (4 M, 5 mL), the mixture was stirred at 25° C. for 3 hours. LCMS showed 25% of the starting material remained and a peak (74%) with desired mass. The reaction mixture was poured into ice water (100 mL), NaHCO₃ solution was added to adjust pH>7, the resulting mixture was extracted with EtOAc (100 mL×3). The combined organic layers were dried over Na₂SO₄, filtered and concentrated in vacuum to afford 2-(4-(2-(2,6-dioxopiperidin-3-yl)-1,3-dioxoisoindolin-4-yl)piperazin-1-yl)acetaldehyde (550 mg) as a yellow solid, which was used directly. MS(M+H₂O+H)⁺=403.0

### Step 6. Synthesis of 4-(4-(2-(((1s,4s)-4-(dibenzylamino)cyclohexyl)(methyl)amino)ethyl)piperazin-1-yl)-2-(2,6-dioxopiperidin-3-yl)isoindoline-1,3-dione (cis) (5)

To a solution of (1s,4s)-N1,N1-dibenzyl-N4-methylcyclohexane-1,4-diamine (cis) (110.34 mg, 357.72 μmol) in DCE (10 mL) were added 2-(4-(2-(2,6-dioxopiperidin-3-yl)-1,3-dioxoisoindolin-4-yl)piperazin-1-yl)acetaldehyde (275 mg, 715.43 μmol) in DMF (2 mL) and AcOH (4.30 mg, 71.54 μmol, 4.09 μL), the mixture was stirred at 25° C. for 30 minutes, to the mixture was added NaBH(OAc)₃ (454.89 mg, 2.15 mmol) at 5° C., the resulting mixture was stirred at 25° C. for 12 hours. LCMS showed the 2-(4-(2-(2,6-dioxopiperidin-3-yl)-1,3-dioxoisoindolin-4-yl)piperazin-1-yl)acetaldehyde was consumed completely and desired mass was detected. The reaction mixture was diluted with H₂O (100 mL), to the mixture was added NaHCO₃ solution to adjust pH>7, the mixture was extracted with EtOAc (100 mL×4). To the combined organic layers was added FA to adjust pH<7, the organic phase was concentrated in vacuum at 35° C. The residue was combined with another batch for further purification. The residue was diluted with DMF and to the mixture was added DIPEA to adjust pH>7, the resulting mixture was filtered and the filtrate was purified by prep-HPLC (column: Waters Xbridge C18 150*50 mm*10 μm; mobile phase: [water(NH₄HCO₃)-ACN]; B %: 62%-92%, 11 min) followed by prep-HPLC (column: Waters Xbridge C18 150*50 mm*10 μm; mobile phase: [water(NH₄HCO₃)-ACN]; B %: 50%-80%, 11 min), the eluent was freeze-dried to afford 4-(4-(2-(((1s,4s)-4-(dibenzylamino)cyclohexyl)(methyl)amino)ethyl)piperazin-1-yl)-2-(2,6-dioxopiperidin-3-yl)isoindoline-1,3-dione (cis) (135 mg, 199.45 μmol, 27.88% yield) as a green solid. MS(M+H)⁺=677.2

### Step 7. Synthesis of 4-(4-(2-(((1s,4s)-4-aminocyclohexyl)(methyl)amino)ethyl)piperazin-1-yl)-2-(2,6-dioxopiperidin-3-yl)isoindoline-1,3-dione (cis) (6)

To a solution of 4-(4-(2-(((1s,4s)-4-(dibenzylamino)cyclohexyl)(methyl)amino)ethyl)piperazin-1-yl)-2-(2,6-dioxopiperidin-3-yl)isoindoline-1,3-dione (cis) (135 mg, 199.45 μmol) in CF₃CH₂OH (20 mL) were added HCl (12 M, 8.31 μL), Pd(OH)₂/C (100 mg, 10% purity) and Pd/C (100 mg, 10% purity) at N₂ atmosphere, the suspension was degassed under vacuum and purged with H₂ several times. The mixture was stirred under H₂ (15 psi) at 60° C. for 16 hours. LCMS showed the starting material was consumed completely and desired mass was detected. The reaction mixture was filtered and the filter cake was washed with CF₃CH₂OH (30 mL), the filtrate was concentrated in vacuum to afford 4-(4-(2-(((1s,4s)-4-aminocyclohexyl)(methyl)amino)ethyl)piperazin-1-yl)-2-(2,6-dioxopiperidin-3-yl)isoindoline-1,3-dione (cis) (130 mg, HCl salt) as a yellow gum, which was used to next step directly. MS(M+H)⁺=497.1

### Step 8. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1s,4s)-4-((2-(4-(2-(2,6-dioxopiperidin-3-yl)-1,3-dioxoisoindolin-4-yl)piperazin-1-yl)ethyl)(methyl)amino)cyclohexyl)-3-methoxybenzamide (cis) (Compound 128)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (80 mg, 178.80 μmol) in DMF (3 mL) were added HATU (101.98 mg, 268.20 μmol) and DIPEA (115.54 mg, 893.99 μmol, 155.72 μL), the mixture was stirred at 25° C. for 15 minutes, to the mixture was added 4-(4-(2-(((1s,4s)-4-aminocyclohexyl)(methyl)amino)ethyl)piperazin-1-yl)-2-(2,6-dioxopiperidin-3-yl)isoindoline-1,3-dione (cis) (128.67 mg, crude, HCl salt), the mixture was stirred at 25° C. for 1 hour. LCMS showed 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid was consumed completely and a peak (35%) with desired mass. To the reaction mixture was added CH₃COOH to adjust pH<7. The mixture was purified by prep-HPLC (column: Phenomenex C18 75*30 mm*3 μm; mobile phase: [water(FA)-ACN]; B %: 18%-48%, 7 min) followed by prep-HPLC (column: Waters Xbridge 150*25 mm*5 μm; mobile phase: [water(NH₄HCO₃)-ACN]; B %: 48%-78%, 8 min), the eluent was freeze-dried to afford 4-((9-cyclopentyl-7,7-difluoro-5-methyl-6-oxo-6,7,8,9-tetrahydro-5H-pyrimido[4,5-b][1,4]diazepin-2-yl)amino)-N-((1 s,4s)-4-((2-(4-(2-(2,6-dioxopiperidin-3-yl)-1,3-dioxoisoindolin-4-yl)piperazin-1-yl)ethyl)(methyl)amino)cyclohexyl)-3-methoxybenzamide (cis) (7.3 mg, 7.33 μmol, 4.10% yield, 93% purity) as a yellow solid. MS(M+H)⁺=926.4

¹H NMR (400 MHz, DMSO-d₆) δ=11.21-10.95 (m, 1H), 8.31-8.19 (m, 2H), 8.02 (d, J=7.1 Hz, 1H), 7.95 (s, 1H), 7.73-7.64 (m, 1H), 7.49 (dd, J=2.6, 4.4 Hz, 2H), 7.33 (dd, J=7.9, 10.5 Hz, 2H), 5.08 (dd, J=5.6, 12.7 Hz, 1H), 4.82-4.69 (m, 1H), 4.03 (t, J=13.9 Hz, 2H), 3.93 (s, 4H), 3.32 (s, 3H), 3.29 (dd, J=2.2, 3.6 Hz, 4H), 2.91-2.82 (m, 1H), 2.65-2.55 (m, 8H), 2.47-2.35 (m, 3H), 2.24 (s, 3H), 2.06-1.99 (m, 1H), 1.97-1.90 (m, 2H), 1.87-1.75 (m, 4H), 1.74-1.67 (m, 2H), 1.60-1.54 (m, 4H), 1.55-1.44 (m, 4H).

### Example 129. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-((2-(4-(2-(2,6-dioxopiperidin-3-yl)-1,3-dioxoisoindolin-4-yl)piperazin-1-yl)ethyl)(methyl)amino)cyclohexyl)-3-methoxybenzamide (trans) (Compound 129)

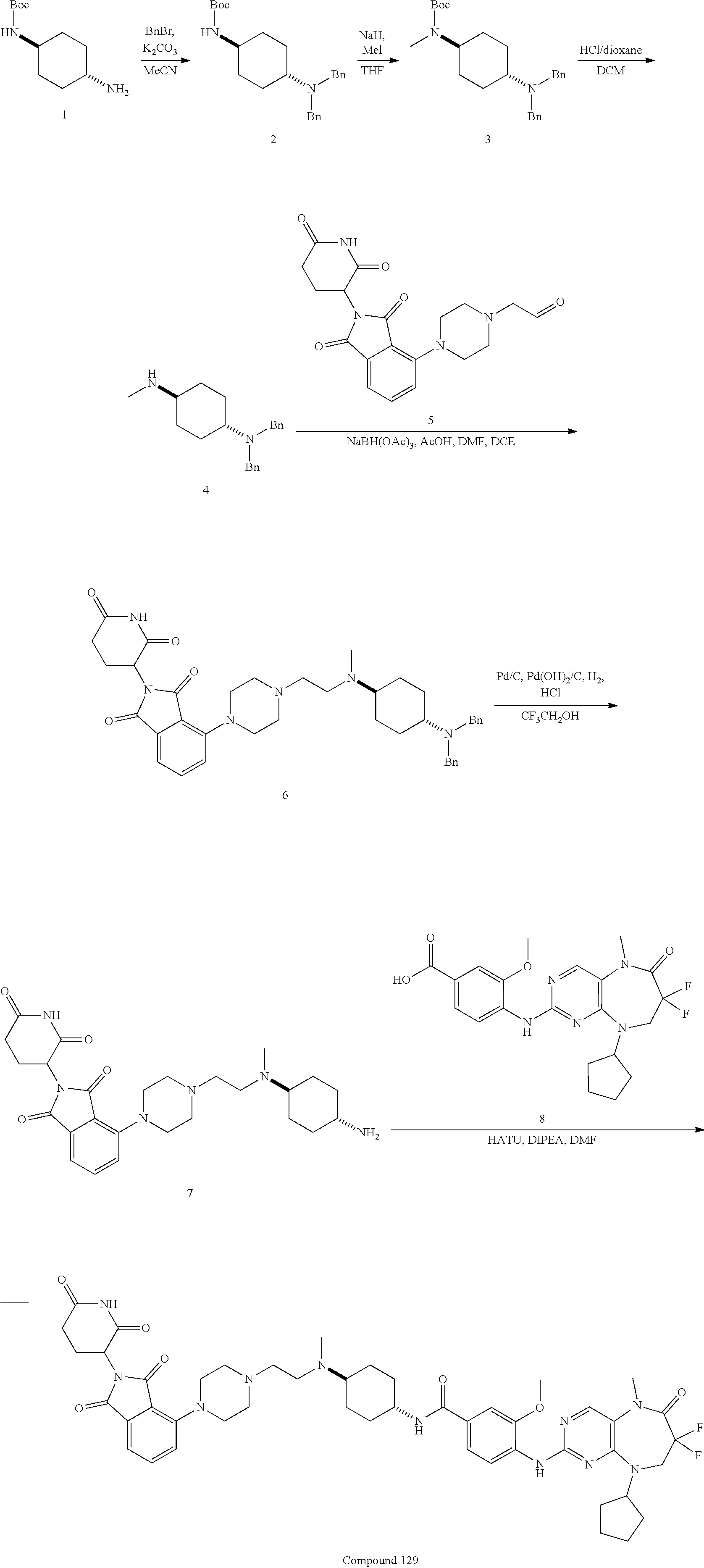

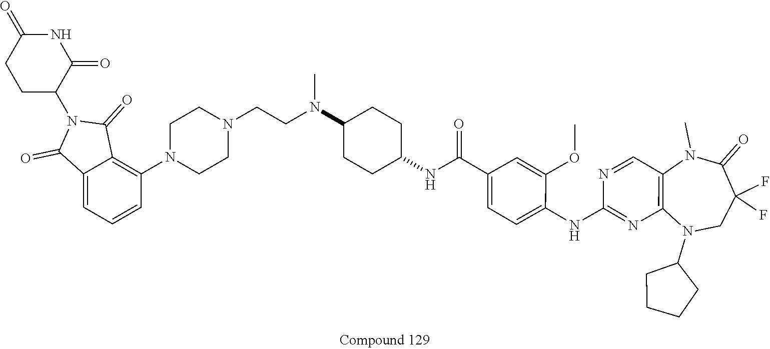

### Step 1. Synthesis of tert-butyl ((1r,4r)-4-(dibenzylamino)cyclohexyl)carbamate (trans) (2)

To a solution of tert-butyl ((1r,4r)-4-aminocyclohexyl)carbamate (trans) (5.00 g, 23.33 mmol) in ACN (80 mL) was added BnBr (7.98 g, 46.66 mmol, 5.54 mL) and K₂CO₃ (12.90 g, 93.33 mmol). The mixture was stirred at 60° C. for 5 h. LCMS showed tert-butyl ((1r,4r)-4-aminocyclohexyl)carbamate (trans) was consumed completely, and a peak (˜53%) with desired mass. The reaction mixture was filtered and filter cake was washed with ACN (100 mL), the filtrate was concentrated in vacuo to afford tert-butyl ((1r,4r)-4-(dibenzylamino)cyclohexyl)carbamate (trans) (6 g, 14.90 mmol, 63.88% yield, 98% purity) as a white solid. MS(M+H)⁺=395.1

### Step 2. Synthesis of tert-butyl ((1r,4r)-4-(dibenzylamino)cyclohexyl)(methyl)carbamate (trans) (3)

To a solution of tert-butyl ((1r,4r)-4-(dibenzylamino)cyclohexyl)carbamate (trans) (5.90 g, 14.95 mmol) in THF (70 mL) was added NaH (897.14 mg, 22.43 mmol, 60% purity) at 0° C., after stirring for 15 minutes, MeI (3.18 g, 22.43 mmol, 1.40 mL) was added at 0° C. The resulting mixture was stirred at 25° C. for 2 h. LCMS showed tert-butyl ((1r,4r)-4-(dibenzylamino)cyclohexyl)carbamate (trans) was consumed completely and one main peak with desired mass. The reaction mixture was quenched by addition of water (400 mL) at 0° C., then extracted with EtOAc (100 mL×3). The combined organic layers were washed with brine (100 mL), dried over anhydrous sodium sulfate, filtered and concentrated under reduced pressure. The residue was purified by column chromatography (SiO₂, Petroleum ether/EtOAc=1/0 to 3/10) to afford tert-butyl ((1r,4r)-4-(dibenzylamino)cyclohexyl)(methyl)carbamate (trans) (5.2 g, 12.60 mmol, 84.26% yield, 99% purity) as a white solid. MS(M+H)⁺=409.9

### Step 3. Synthesis of (1r,4r)-N1,N1-dibenzyl-N4-methylcyclohexane-1,4-diamine (trans) (4)

A mixture of tert-butyl ((1r,4r)-4-(dibenzylamino)cyclohexyl)(methyl)carbamate (trans) (5.00 g, 12.24 mmol) and HCl/dioxane (4 M, 3.06 mL) in DCM (25 mL) was stirred at 25° C. for 1 h. LCMS showed tert-butyl ((1r,4r)-4-(dibenzylamino)cyclohexyl)(methyl)carbamate (trans) was consumed completely and one main peak with desired mass. The reaction mixture was concentrated under reduced pressure. The residue was adjusted to pH=8 with saturated sodium bicarbonate solution and extracted with DCM (100 mL×3). The combined organic layers were washed with brine (100 mL), dried over anhydrous sodium sulfate, filtered and concentrated under reduced pressure to afford (1r,4r)-N1,N1-dibenzyl-N4-methylcyclohexane-1,4-diamine (trans) (3.5 g, 11.35 mmol, 92.72% yield) as a white solid. MS(M+H)⁺=309.2

### Step 4. Synthesis of 4-(4-(2-(((1r,4r)-4-(dibenzylamino)cyclohexyl)(methyl)amino)ethyl)piperazin-1-yl)-2-(2,6-dioxopiperidin-3-yl)isoindoline-1,3-dione(trans) (6)

To a solution of (1r,4r)-N1,N1-dibenzyl-N4-methylcyclohexane-1,4-diamine (trans) (110.34 mg, 357.72 μmol) in DCE (10 mL) were added 2-(4-(2-(2,6-dioxopiperidin-3-yl)-1,3-dioxoisoindolin-4-yl)piperazin-1-yl)acetaldehyde (275 mg, 715.43 μmol) in DMF (2 mL) and AcOH (4.30 mg, 71.54 μmol, 4.09 μL), the mixture was stirred at 25° C. for 30 minutes, to the mixture was added NaBH(OAc)₃ (454.89 mg, 2.15 mmol) at 5° C., the suspension was stirred at 25° C. for 12 hours. LCMS showed the (1r,4r)-N1,N1-dibenzyl-N4-methylcyclohexane-1,4-diamine(trans) was consumed completely and 96% of desired mass was detected. The reaction mixture was diluted with H₂O (100 mL), to the mixture was added NaHCO₃ solution to adjust pH>7, the mixture was extracted with EtOAc (100 mL×4). To the combined organic layers was concentrated in vacuum at 35° C. The residue was purified by prep-HPLC (column: Waters Xbridge C18 150×50 mm×10 μm; mobile phase: [water(NH₄HCO₃)-ACN]; B %: 50%-80%, 11 min), the eluent was lyophilized to afford 4-(4-(2-(((1r,4r)-4-(dibenzylamino)cyclohexyl)(methyl)amino)ethyl)piperazin-1-yl)-2-(2,6-dioxopiperidin-3-yl)isoindoline-1,3-dione (trans) (150 mg, 221.62 μmol, 30.98% yield) as a yellow solid. MS(M+H)⁺=677.3

### Step 5. Synthesis of 4-(4-(2-(((1r,4r)-4-aminocyclohexyl)(methyl)amino)ethyl)piperazin-1-yl)-2-(2,6-dioxopiperidin-3-yl)isoindoline-1,3-dione(trans) (7)

To a solution of 4-(4-(2-(((1r,4r)-4-(dibenzylamino)cyclohexyl)(methyl)amino)ethyl)piperazin-1-yl)-2-(2,6-dioxopiperidin-3-yl)isoindoline-1,3-dione (trans) (120 mg, 177.29 μmol) in CF₃CH₂OH (5 mL) was added Pd/C (10%, 20 mg) and Pd(OH)₂/C (10%, 20 mg), HCl (12 M, 73.87 μL) under N₂ atmosphere. The suspension was degassed and purged with H₂ for 3 times. The mixture was stirred under H₂ (15 Psi) at 60° C. for 16 h. LCMS showed 4-(4-(2-(((1r,4r)-4-(dibenzylamino)cyclohexyl)(methyl)amino)ethyl)piperazin-1-yl)-2-(2,6-dioxopiperidin-3-yl)isoindoline-1,3-dione was consumed completely, 9% m/z=587.5, 19% of toluene, 73% desired mass was detected. The reaction mixture was filtered and filter cake was washed with EtOAc (50 mL), the filtrate was concentrated in vacuo to afford 4-(4-(2-(((1r,4r)-4-aminocyclohexyl)(methyl)amino)ethyl)piperazin-1-yl)-2-(2,6-dioxopiperidin-3-yl)isoindoline-1,3-dione (trans) (85 mg, 159.46 μmol, 89.94% yield, HCl salt) as a yellow solid. MS(M+H)⁺=497.4

### Step 6. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-((2-(4-(2-(2,6-dioxopiperidin-3-yl)-1,3-dioxoisoindolin-4-yl)piperazin-1-yl)ethyl)(methyl)amino)cyclohexyl)-3-methoxybenzamide (trans) (Compound 129)

To a solution of 4-(4-(2-(((1r,4r)-4-aminocyclohexyl)(methyl)amino)ethyl)piperazin-1-yl)-2-(2,6-dioxopiperidin-3-yl)isoindoline-1,3-dione (trans) (85 mg, 171.16 μmol) and 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (76.58 mg, 171.16 μmol) in DMF (3 mL) was added HATU (97.62 mg, 256.75 μmol) and DIPEA (66.37 mg, 513.49 μmol, 89.44 μL). The mixture was stirred at 25° C. for 2 h. LCMS showed 4-(4-(2-(((1r,4r)-4-aminocyclohexyl)(methyl)amino)ethyl)piperazin-1-yl)-2-(2,6-dioxopiperidin-3-yl)isoindoline-1,3-dione was consumed completely, a peak (69%) with desired mass. The reaction mixture was diluted with water (50 mL) and extracted with EtOAc (20 mL×3). The combined organic layers were washed with brine (20 mL), dried over anhydrous sodium sulfate, filtered and concentrated under reduced pressure. The residue was purified by column chromatography (SiO₂, MeOH/EtOAc=0/1 to 1/10) followed by prep-HPLC (neutral condition: column: Waters Xbridge 150×25 mm×5 μm; mobile phase: [water(NH₄HCO₃)-ACN]; B %: 40%-70%, 10 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-((2-(4-(2-(2,6-dioxopiperidin-3-yl)-1,3-dioxoisoindolin-4-yl)piperazin-1-yl)ethyl)(methyl)amino)cyclohexyl)-3-methoxybenzamide (33.3 mg, 33.80 μmol, 19.75% yield, 93.8% purity) as a yellow solid. MS(M+H)⁺=926.6.

¹H NMR (400 MHz, DMSO-d₆) δ=11.18-10.97 (m, 1H), 8.30-8.21 (m, 2H), 8.08-8.04 (m, 1H), 7.96 (s, 1H), 7.71 (dd, J=7.2, 8.3 Hz, 1H), 7.53-7.44 (m, 2H), 7.35 (t, J=7.7 Hz, 2H), 5.09 (dd, J=5.4, 12.8 Hz, 1H), 4.83-4.71 (m, 1H), 4.05-3.98 (m, 2H), 3.94 (s, 3H), 3.80-3.68 (m, 1H), 3.33 (s, 3H), 3.32-3.29 (m, 4H), 2.93-2.83 (m, 1H), 2.64-2.54 (m, 8H), 2.46-2.37 (m, 3H), 2.22 (s, 3H), 2.06-1.88 (m, 5H), 1.81-1.68 (m, 4H), 1.65-1.55 (m, 4H), 1.45-1.31 (m, 4H).

### Example 130. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1-oxoisoindolin-5-yl)piperazin-1-yl)propanoyl)piperidin-4-yl)-3-methoxybenzamide (Compound 130)

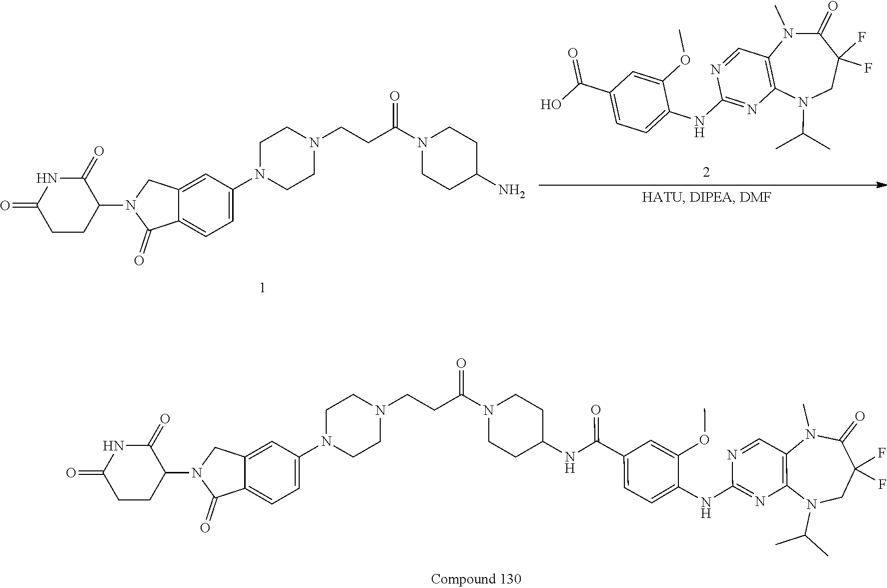 [SMILES: NC1CCN(C(=O)CCN2CCN(c3ccc4c(c3)CN(C3CCC(=O)NC3=O)C4=O)CC2)CC1 ; COc1cc(C(=O)O)ccc1Nc1ncc2c(n1)N(C(C)C)CC(F)(F)C(=O)N2C ; COc1cc(C(=O)NC2CCN(C(=O)CCN3CCN(c4ccc5c(c4)CN(C4CCC(=O)NC4=O)C5=O)CC3)CC2)ccc1Nc1ncc2c(n1)N(C(C)C)CC(F)(F)C(=O)N2C]

The compound 130 was synthesized by the method described in the scheme similar to the method described in Example 44.

MS(M+H)⁺=886.4, ¹H NMR (400 MHz, DMSO-d₆) δ=10.95 (s, 1H), 8.32 (d, J=8.3 Hz, 1H), 8.23 (s, 1H), 8.16 (d, J=8.0 Hz, 1H), 7.89 (s, 1H), 7.56-7.48 (m, 3H), 7.14-7.04 (m, 2H), 5.06 (dd, J=4.8, 13.3 Hz, 1H), 4.93-4.84 (m, 1H), 4.44-4.30 (m, 2H), 4.26-4.18 (m, 1H), 4.12-3.96 (m, 4H), 3.94 (s, 3H), 3.32-3.24 (m, 7H), 3.19-3.11 (m, 1H), 2.97-2.91 (m, 1H), 2.72-2.70 (m, 1H), 2.62-2.53 (m, 9H), 2.44-2.36 (m, 1H), 2.01-1.93 (m, 1H), 1.91-1.78 (m, 2H), 1.55-1.38 (m, 2H), 1.26 (s, 3H), 1.24 (s, 3H).

### Example 131. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(4-(2-(2,6-dioxopiperidin-3-yl)-1,3-dioxoisoindolin-4-yl)piperazin-1-yl)cyclohexyl)-3-methoxybenzamide (trans) (Compound 131)

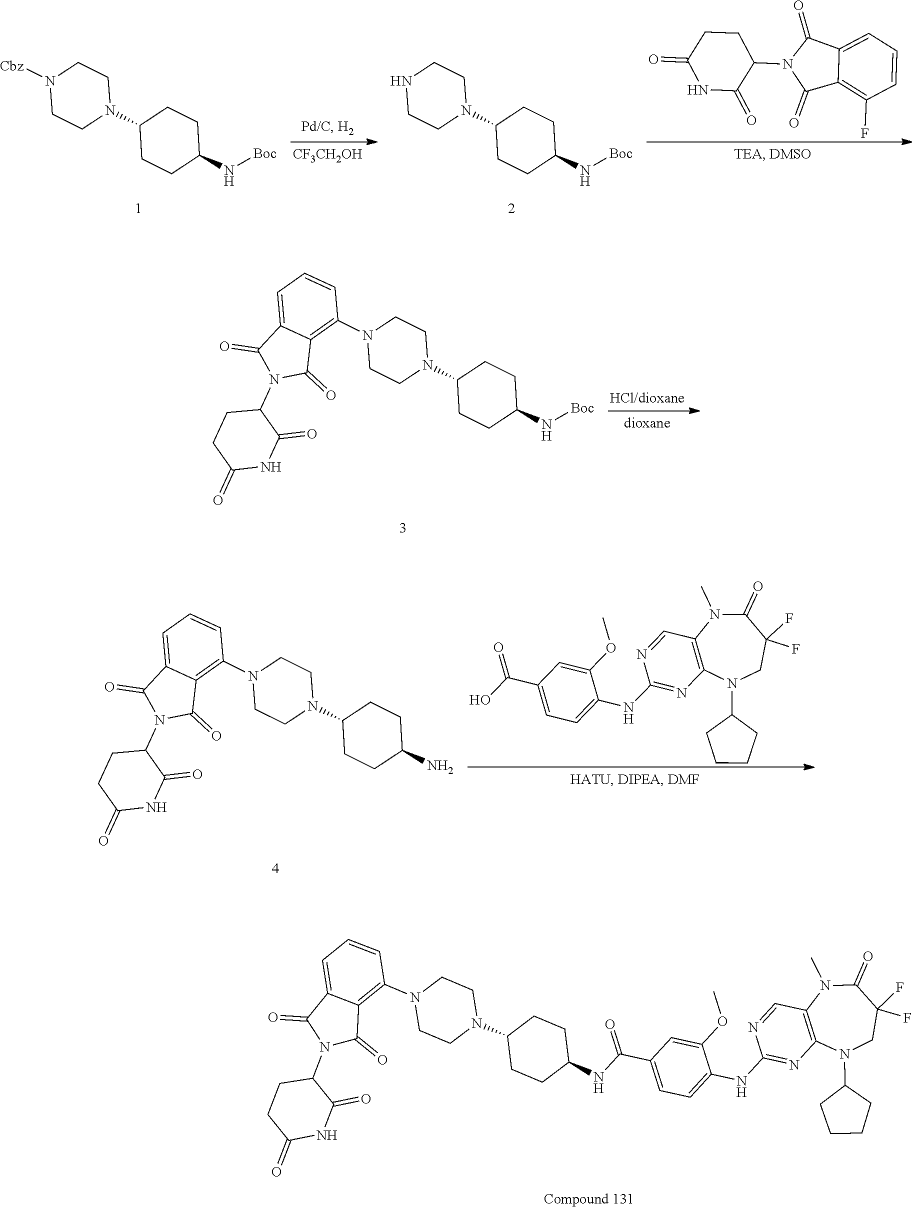

The compound 131 was synthesized by the method described in the scheme similar to the method described in Example 51.

MS(M+H)⁺=869.3, ¹H NMR (400 MHz, DMSO-d₆) δ=11.09 (s, 1H), 8.31-8.17 (m, 3H), 8.09 (d, J=7.9 Hz, 1H), 7.96 (s, 1H), 7.74-7.66 (m, 1H), 7.52-7.44 (m, 2H), 7.34 (t, J=7.6 Hz, 2H), 5.09 (dd, J=5.4, 12.8 Hz, 1H), 4.82-4.70 (m, 1H), 4.04 (t, J=14.0 Hz, 2H), 3.93 (s, 3H), 3.85-3.62 (m, 2H), 3.32 (s, 3H), 3.30-3.28 (m, 4H), 2.93-2.82 (m, 1H), 2.75-2.68 (m, 4H), 2.63-2.54 (m, 2H), 2.09-1.99 (m, 1H), 1.98-1.85 (m, 6H), 1.77-1.67 (m, 2H), 1.66-1.54 (m, 4H), 1.47-1.31 (m, 4H)

### Example 132. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-((1-(2-(2,6-dioxopiperidin-3-yl)-1,3-dioxoisoindolin-4-yl)piperidin-4-yl)oxy)phenyl)-2-fluoro-5-methoxybenzamide (Compound 132)

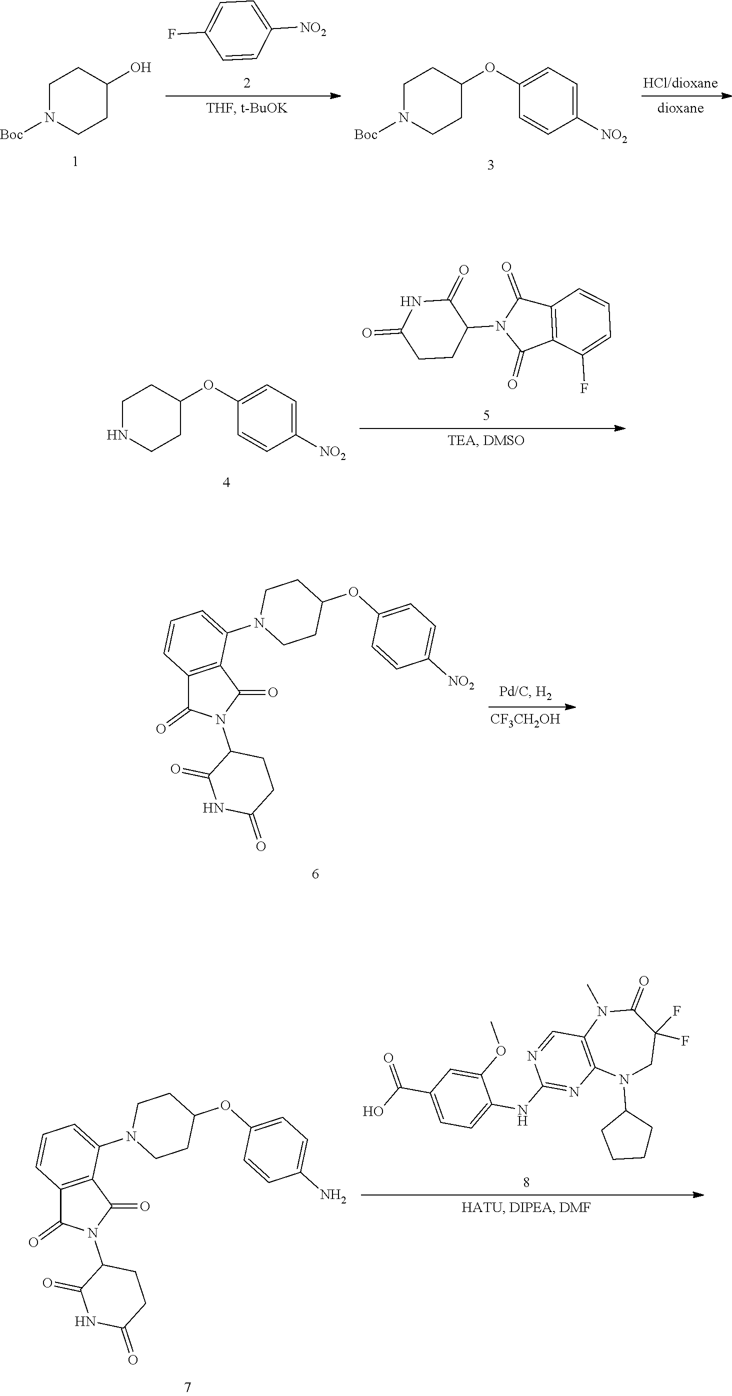

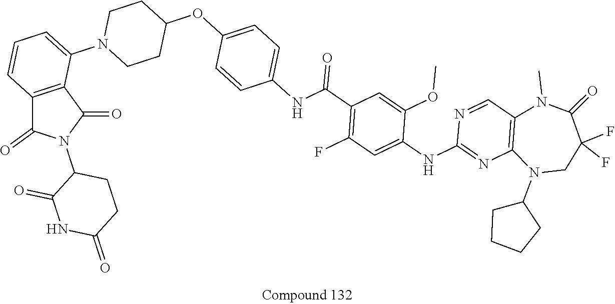 [SMILES: COc1cc(C(=O)Nc2ccc(OC3CCN(c4cccc5c4C(=O)N(C4CCC(=O)NC4=O)C5=O)CC3)cc2)c(F)cc1Nc1ncc2c(n1)N(C1CCCC1)CC(F)(F)C(=O)N2C]

### Step 1. Synthesis of tert-butyl 4-(4-nitrophenoxy)piperidine-1-carboxylate (3)

To a solution of 1-fluoro-4-nitro-benzene (1.05 g, 7.45 mmol, 790.69 μL) in THF (20 mL) were added tert-butyl 4-hydroxypiperidine-1-carboxylate (1 g, 4.97 mmol) and t-BuOK (1.12 g, 9.94 mmol) slowly at 20° C. under N₂ and the resulting mixture was stirred at 20° C. for 0.5 h. LCMS showed starting material was consumed completely and a peak (74%) with desired mass (−100). The reaction mixture was diluted with H₂O (40 mL) and extracted with EtOAc (40 mL×3). The organic layer was dried over Na₂SO₄, filtered and concentrated. The residue was purified by flash silica gel chromatography (10 g SepaFlash® Silica Flash Column, Eluent of 0˜33% EtOAc/Petroleum ether gradient @100 mL/min) to afford tert-butyl 4-(4-nitrophenoxy)piperidine-1-carboxylate (1.4 g, 3.95 mmol, 79.54% yield, 91% purity) as a yellow oil. MS(M−100+H)⁺=223.1

### Step 2. Synthesis of 4-(4-nitrophenoxy)piperidine (4)

To a solution of tert-butyl 4-(4-nitrophenoxy)piperidine-1-carboxylate (500 mg, 1.55 mmol) in dioxane (5 mL) was added HCl/dioxane (4 M, 12 mL) at 20° C. and the resulting mixture was stirred at 20° C. for 0.5 h. LCMS showed starting material was consumed completely and a main peak (100%) with desired mass. The reaction mixture was concentrated in vacuum to afford 4-(4-nitrophenoxy)piperidine (402 mg, crude, HCl salt) as a white solid. MS(M+H)⁺=223.2

### Step 3. Synthesis of 2-(2,6-dioxopiperidin-3-yl)-4-(4-(4-nitrophenoxy)piperidin-1-yl)isoindoline-1,3-dione (6)

To a solution of 2-(2,6-dioxo-3-piperidyl)-4-fluoro-isoindoline-1,3-dione (450 mg, 1.63 mmol) and 4-(4-nitrophenoxy)piperidine (398.27 mg, crude) in DMSO (8 mL) was added TEA (494.55 mg, 4.89 mmol) at 20° C. and the resulting mixture was stirred at 100° C. for 16 h. LCMS showed starting material was consumed completely and a peak (84%) with desired mass. The reaction mixture was diluted with H₂O (30 mL) and extracted with EtOAc (30 mL×3). The organic layer was washed with brine (30 mL×3), dried over Na₂SO₄, filtered and concentrated. The residue was purified by flash silica gel chromatography (10 g SepaFlash® Silica Flash Column, Eluent of 0˜60% EtOAc/Petroleum ether gradient @100 mL/min) to afford 2-(2,6-dioxopiperidin-3-yl)-4-(4-(4-nitrophenoxy)piperidin-1-yl)isoindoline-1,3-dione (743 mg, 1.55 mmol, 95.32% yield) as a yellow solid. MS(M+H)⁺=478.9

### Step 4. Synthesis of 4-(4-(4-aminophenoxy)piperidin-1-yl)-2-(2,6-dioxopiperidin-3-yl)isoindoline-1,3-dione (7)

To a solution of 2-(2,6-dioxopiperidin-3-yl)-4-(4-(4-nitrophenoxy)piperidin-1-yl)isoindoline-1,3-dione (250 mg, 522.52 μmol) in CF₃CH₂OH (10 mL) was added Pd/C (80 mg, 10% purity) under N₂ atmosphere. The suspension was degassed and purged with H₂ for 3 times. The mixture was stirred under H₂ (15 Psi) at 20° C. for 12 h. LCMS showed starting material was consumed completely and a peak (82%) with desired mass. The reaction mixture was diluted with H₂O (20 mL) and filtered, the filtrate was concentrated in vacuum to afford 4-(4-(4-aminophenoxy)piperidin-1-yl)-2-(2,6-dioxopiperidin-3-yl)isoindoline-1,3-dione (235 mg, crude) as a yellow solid. MS(M+H)⁺=449.3

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-((1-(2-(2,6-dioxopiperidin-3-yl)-1,3-dioxoisoindolin-4-yl)piperidin-4-yl)oxy)phenyl)-2-fluoro-5-methoxybenzamide (Compound 132)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (80 mg, 171.89 μmol) in DMF (3 mL) were added HATU (71.89 mg, 189.07 μmol) and DIPEA (44.43 mg, 343.77 μmol, 59.88 μL). The mixture was stirred at 20° C. for 10 min and a solution of 4-(4-(4-aminophenoxy)piperidin-1-yl)-2-(2,6-dioxopiperidin-3-yl)isoindoline-1,3-dione (92.50 mg, 206.26 μmol) in DMF (3 mL) and DIPEA (44.43 mg, 343.77 μmol, 59.88 μL) was added and the resulting mixture was stirred at 20° C. for 1 h. LCMS showed starting material was consumed completely and a peak (70%) with desired mass. The reaction mixture was diluted with H₂O (16 mL) and extracted with EtOAc (16 mL×3). The organic layer was washed with brine (16 mL×3), dried over Na₂SO₄, filtered and concentrated. The residue was purified by prep-HPLC (column: Unisil 3-100 C18 Ultra 150*50 mm*3 μm; mobile phase: [water(FA)-ACN]; B %: 50%-80%, 10 min) and re-purified by prep-HPLC (column: Waters Xbridge 150*25 mm*5 μm; mobile phase: [water(NH₄HCO₃)-ACN]; B %: 66%-86%, 8 min), the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-((1-(2-(2,6-dioxopiperidin-3-yl)-1,3-dioxoisoindolin-4-yl)piperidin-4-yl)oxy)phenyl)-2-fluoro-5-methoxybenzamide (23.2 mg, 25.38 μmol, 14.76% yield, 98% purity) as a yellow solid. MS(M+H)⁺=896.3

¹H NMR (400 MHz, DMSO-d₆) δ=11.07 (br s, 1H), 10.01 (d, J=1.3 Hz, 1H), 8.37-8.25 (m, 2H), 8.09 (s, 1H), 7.74-7.59 (m, 3H), 7.37 (dd, J=7.7, 15.3 Hz, 2H), 7.27 (d, J=6.6 Hz, 1H), 7.01 (d, J=9.0 Hz, 2H), 5.11 (dd, J=5.4, 12.8 Hz, 1H), 4.90-4.79 (m, 1H), 4.59 (d, J=3.5 Hz, 1H), 4.09 (t, J=13.9 Hz, 2H), 3.95 (s, 3H), 3.63-3.52 (m, 2H), 3.34 (s, 3H), 3.24 (t, J=9.0 Hz, 2H), 2.94-2.82 (m, 1H), 2.63-2.54 (m, 2H), 2.16-2.06 (m, 2H), 2.06-1.94 (m, 3H), 1.89-1.77 (m, 2H), 1.76-1.72 (br s, 2H), 1.69-1.57 (m, 4H).

### Example 133. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1-oxoisoindolin-4-yl)piperazin-1-yl)propanoyl)piperidin-4-yl)-3-methoxybenzamide (Compound 133)

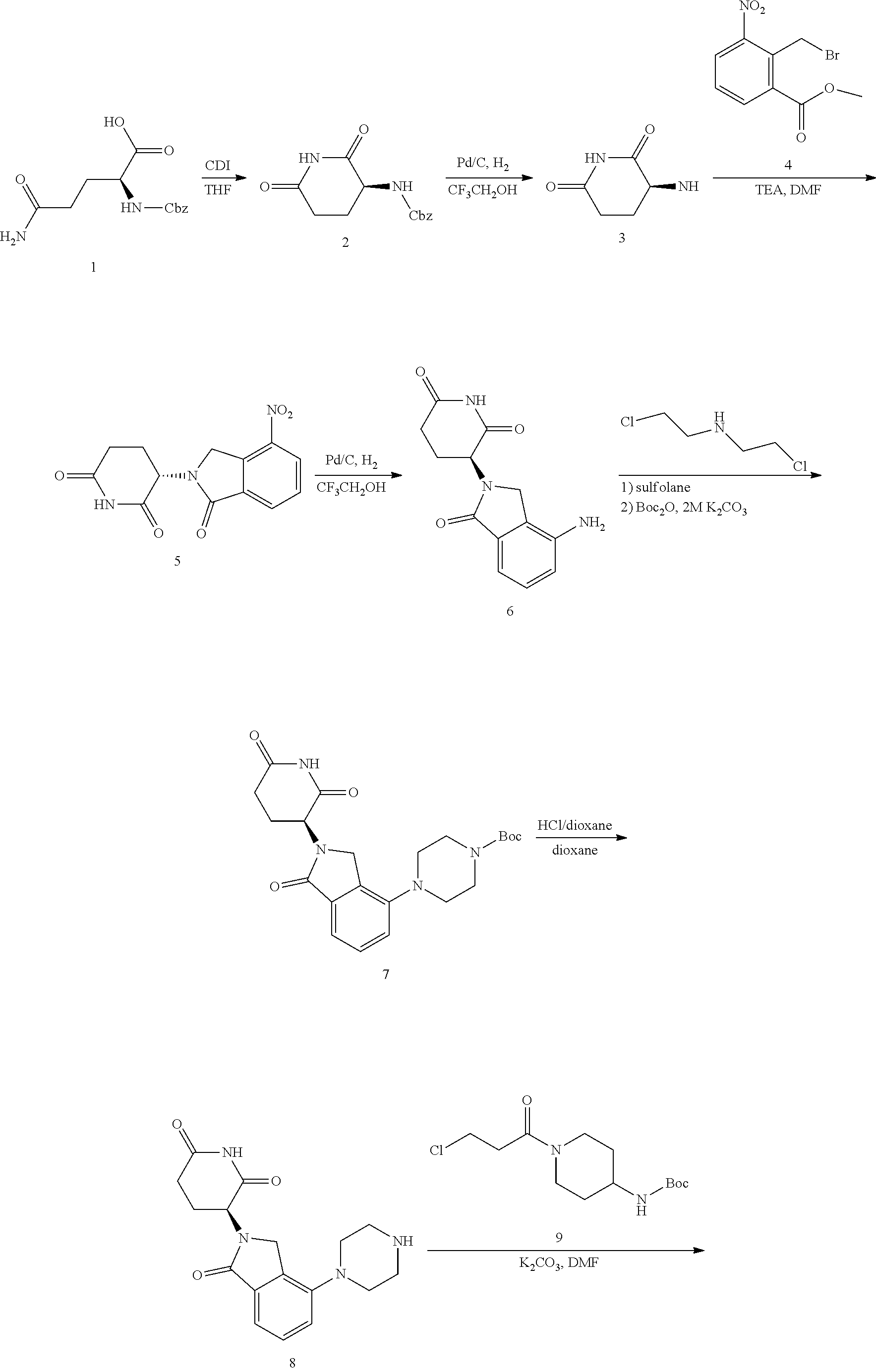

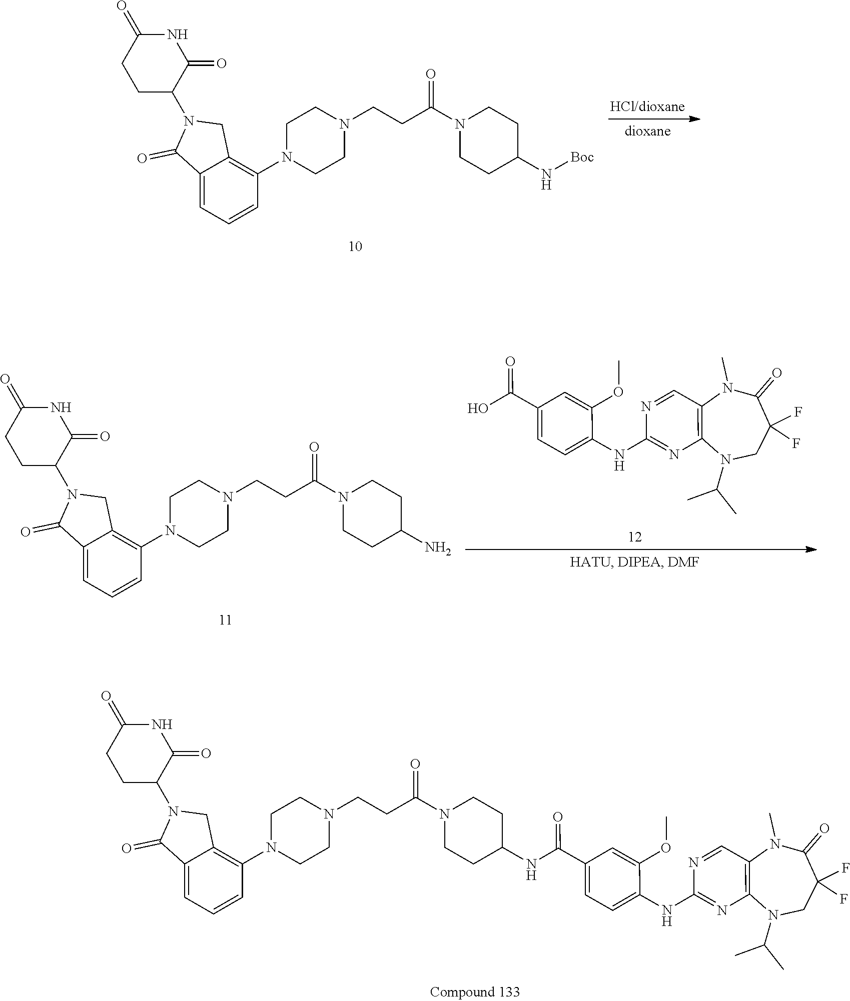 [SMILES: CC(C)(C)OC(=O)NC1CCN(C(=O)CCN2CCN(c3cccc4c3CN(C3CCC(=O)NC3=O)C4=O)CC2)CC1 ; "HCl/dioxane" ; NC1CCN(C(=O)CCN2CCN(c3cccc4c3CN(C3CCC(=O)NC3=O)C4=O)CC2)CC1 ; COc1cc(C(=O)O)ccc1Nc1ncc2c(n1)N(C(C)C)CC(F)(F)C(=O)N2C ; COc1cc(C(=O)NC2CCN(C(=O)CCN3CCN(c4cccc5c4CN(C4CCC(=O)NC4=O)C5=O)CC3)CC2)ccc1Nc1ncc2c(n1)N(C(C)C)CC(F)(F)C(=O)N2C]

### Step 1. Synthesis of benzyl (S)-(2,6-dioxopiperidin-3-yl)carbamate (2)

To a solution of ((benzyloxy)carbonyl)-L-glutamine (25 g, 89.20 mmol) in THF (500 mL) was added CDI (21.70 g, 133.80 mmol). The mixture was stirred at 80° C. for 16 h under N₂ atmosphere. LCMS showed a main peak with desired mass. The reaction mixture was concentrated under reduced pressure and diluted with EtOAc (500 mL), washed with brine (300 mL×3). The organic layer were dried over anhydrous Na₂SO₄, filtered and the filtrate was concentrated under reduced pressure and then triturated with MTBE (300 mL) at 25° C. for 30 min. Then it was filtered and the cake was dried under reduced pressure to afford benzyl (S)-(2,6-dioxopiperidin-3-yl)carbamate (20 g, 71.68 mmol, 80.37% yield, 94% purity) as a white solid. MS(M+H)⁺=263.0

### Step 2. Synthesis of (S)-3-aminopiperidine-2,6-dione (3)

To a solution of benzyl (S)-(2,6-dioxopiperidin-3-yl)carbamate (10 g, 38.13 mmol) in CF₃CH₂OH (300 mL) was added Pd/C (2 g, 10% purity) under N₂ atmosphere, then the mixture was stirred at 20° C. for 12 h under H₂ atmosphere (15 Psi). LCMS showed no peak with desired mass. TLC indicated the starting material was consumed completely and one major new spot with larger polarity was detected. The reaction mixture was filtered and the filtrate was concentrated under reduced pressure to give a residue to afford (S)-3-aminopiperidine-2,6-dione (10 g, crude) as a blue solid.

### Step 3. Synthesis of (S)-3-(4-nitro-1-oxoisoindolin-2-yl)piperidine-2,6-dione (5)

To a solution of methyl 2-(bromomethyl)-3-nitrobenzoate (6.42 g, 23.41 mmol) in DMF (60 mL) was added (S)-3-aminopiperidine-2,6-dione (3 g, crude), TEA (7.11 g, 70.24 mmol, 9.78 mL), the mixture was stirred at 75° C. for 16 h. LCMS showed a peak (91%) with desired mass. The mixture was pour into H₂O (300 mL) and EtOAc (200 mL). Then the mixture was filtered and the cake was dried under reduced pressure. The filter cake was triturated with MTBE (20 mL) at 25° C. for 30 min to afford (S)-3-(4-nitro-1-oxoisoindolin-2-yl)piperidine-2,6-dione (5 g, crude) as a yellow solid. MS(M+H)⁺=290.0

### Step 4. Synthesis of (S)-3-(4-amino-1-oxoisoindolin-2-yl)piperidine-2,6-dione (6)

A mixture of (S)-3-(4-nitro-1-oxoisoindolin-2-yl)piperidine-2,6-dione (5 g, crude) and Pd/C (1 g, 10% purity) in CF₃CH₂OH (100 mL) was degassed and purged with N₂ for 3 times, and then the mixture was stirred at 25° C. for 16 h under H₂ atmosphere. LCMS showed a peak (94%) with desired mass. The reaction mixture was filtered and the filtrate was concentrated under reduced pressure to afford (S)-3-(4-amino-1-oxoisoindolin-2-yl)piperidine-2,6-dione (4 g, crude) as a yellow solid. MS(M+H)⁺=260.0

### Step 5. Synthesis of tert-butyl (S)-4-(2-(2,6-dioxopiperidin-3-yl)-1-oxoisoindolin-4-yl)piperazine-1-carboxylate (7)

A solution of (S)-3-(4-amino-1-oxoisoindolin-2-yl)piperidine-2,6-dione (4 g, crude) and bis(2-chloroethyl)amine (5.51 g, 30.86 mmol, HCl) in sulfolane (40 mL) was stirred at 140° C. for 14 h. LCMS showed (S)-3-(1-oxo-4-(piperazin-1-yl)isoindolin-2-yl)piperidine-2,6-dione mass was detected. The mixture was adjusted to pH=9 with K₂CO₃ (2 M, 10 mL), Boc₂O (4.04 g, 18.51 mmol, 4.25 mL) was added and the mixture was stirred at 25° C. for 2 h. LCMS showed (S)-3-(1-oxo-4-(piperazin-1-yl)isoindolin-2-yl)piperidine-2,6-dione mass remained and 12% tert-butyl (S)-4-(2-(2,6-dioxopiperidin-3-yl)-1-oxoisoindolin-4-yl)piperazine-1-carboxylate mass was detected. K₂CO₃ (2 M, 5.40 mL) was added to pH=9 and the mixture was stirred at 25° C. for 2 h. LCMS showed the desired mass was detected. The mixture was diluted with H₂O (100 mL) and extracted with MTBE (100 mL×3), the combined organic layer was washed with brine (100 mL), dried over Na₂SO₄ and filtered. The filtrate was concentrated under reduced pressure. The crude was diluted with MTBE (50 mL) and stirred at 25° C. for 1 h. The mixture was filtered and the filter cake was washed with MTBE (50 mL). The filter cake was collected to afford tert-butyl (S)-4-(2-(2,6-dioxopiperidin-3-yl)-1-oxoisoindolin-4-yl)piperazine-1-carboxylate (1.07 g, 2.40 mmol, 15.54% yield, 96% purity) as yellow solid. The filtrate was concentrated under reduced pressure and then purified by flash silica gel chromatography (40 g SepaFlash® Silica Flash Column, Eluent of 50˜90% EtOAc/Petroleum ether gradient @50 mL/min) to afford tert-butyl (S)-4-(2-(2,6-dioxopiperidin-3-yl)-1-oxoisoindolin-4-yl)piperazine-1-carboxylate (1.43 g, 2.90 mmol, 18.82% yield, 87% purity) as yellow solid. MS(M+H)⁺=429.2

### Step 6. Synthesis of (S)-3-(1-oxo-4-(piperazin-1-yl)isoindolin-2-yl)piperidine-2,6-dione (8)

To a solution of tert-butyl (S)-4-(2-(2,6-dioxopiperidin-3-yl)-1-oxoisoindolin-4-yl)piperazine-1-carboxylate (1.07 g, 2.50 mmol) in dioxane (10 mL) was added HCl/dioxane (4 M, 10 mL) and the mixture was stirred at 25° C. for 1 h. LCMS showed 100% of the desired mass was detected after work up. The mixture was concentrated under reduced pressure to afford (S)-3-(1-oxo-4-(piperazin-1-yl)isoindolin-2-yl)piperidine-2,6-dione (0.9 g, crude, HCl) as yellow solid. MS(M+H)⁺=329.1

### Step 7. Synthesis of tert-butyl (1-(3-(4-(2-(2,6-dioxopiperidin-3-yl)-1-oxoisoindolin-4-yl)piperazin-1-yl)propanoyl)piperidin-4-yl)carbamate (10)

To a solution of (S)-3-(1-oxo-4-(piperazin-1-yl)isoindolin-2-yl)piperidine-2,6-dione (0.8 g, crude, HCl) in DMF (10 mL) were added K₂CO₃ (912.00 mg, 6.60 mmol) and tert-butyl (1-(3-chloropropanoyl)piperidin-4-yl)carbamate (1.28 g, 4.39 mmol) and the mixture was stirred at 80° C. for 14 h. LCMS showed the desired mass was detected. The mixture was diluted with H₂O (10 mL) and extracted with EtOAc (10 mL×3), the combined organic layer was washed with brine (10 mL×3), dried over Na₂SO₄ and filtered. The filtrate was concentrated under reduced pressure. The residue was purified by flash silica gel chromatography (12 g SepaFlash® Silica Flash Column, Eluent of 0˜50% EtOH/EtOAc gradient @50 mL/min) to afford tert-butyl (1-(3-(4-(2-(2,6-dioxopiperidin-3-yl)-1-oxoisoindolin-4-yl)piperazin-1-yl)propanoyl)piperidin-4-yl)carbamate (720 mg, 1.16 mmol, 52.97% yield, 94% purity) as yellow solid. MS(M+H)⁺=583.4

### Step 8. Synthesis of 3-(4-(4-(3-(4-aminopiperidin-1-yl)-3-oxopropyl)piperazin-1-yl)-1-oxoisoindolin-2-yl)piperidine-2,6-dione (11)

To a solution of tert-butyl (1-(3-(4-(2-(2,6-dioxopiperidin-3-yl)-1-oxoisoindolin-4-yl)piperazin-1-yl)propanoyl)piperidin-4-yl)carbamate (0.7 g, 1.20 mmol) in dioxane (10 mL) was added HCl/dioxane (4 M, 10.45 mL) and the mixture was stirred at 25° C. for 0.5 h. LCMS showed 89% of the desired mass was detected after work up. The mixture was concentrated under reduced pressure to afford 3-(4-(4-(3-(4-aminopiperidin-1-yl)-3-oxopropyl)piperazin-1-yl)-1-oxoisoindolin-2-yl)piperidine-2,6-dione (630 mg, crude, HCl) as yellow solid. MS(M+H)⁺=483.1

### Step 9. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1-oxoisoindolin-4-yl)piperazin-1-yl)propanoyl)piperidin-4-yl)-3-methoxybenzamide (Compound 133)

To a solution of 4-((7,7-difluoro-9-isopropyl-5-methyl-6-oxo-6,7,8,9-tetrahydro-5H-pyrimido[4,5-b][1, 4]diazepin-2-yl)amino)-3-methoxybenzoic acid (370 mg, 878.03 μmol) in DMF (5 mL) was added HATU (400.62 mg, 1.05 mmol) and DIPEA (137.27 mg, 1.06 mmol, 185.00 μL) and the mixture was stirred at 25° C. for 15 min. Then a solution of 3-(4-(4-(3-(4-aminopiperidin-1-yl)-3-oxopropyl)piperazin-1-yl)-1-oxoisoindolin-2-yl)piperidine-2,6-dione (595 mg, crude, 2HCl) and DIPEA (274.54 mg, 2.12 mmol, 370.00 L) in DMF (5 mL) was added and the mixture was stirred at 25° C. for 1 h. LCMS showed 71% of the desired mass was detected. The mixture was diluted with H₂O (20 mL) and extracted with EtOAc (10 mL×3), the combined organic layer was washed with H₂O (10 mL), dried over Na₂SO₄ and filtered. The filtrate was concentrated under reduced pressure. The residue was purified by prep-HPLC (column: Waters Xbridge C18 150*50 mm*10 μm; mobile phase: [water(NH₄HCO₃)-ACN]; B %: 24%-54%, 10 min) and the eluent was lyophilized to afford 4-((7,7-difluoro-9-isopropyl-5-methyl-6-oxo-6,7,8,9-tetrahydro-5H-pyrimido[4,5-b][1, 4]diazepin-2-yl)amino)-N-(1-(3-(4-(2-(2,6-dioxopiperidin-3-yl)-1-oxoisoindolin-4-yl)piperazin-1-yl)propanoyl)piperidin-4-yl)-3-methoxybenzamide (383 mg, 428.84 μmol, 48.84% yield, 99.2% purity) as white solid. MS(M+H)⁺=886.6

¹H NMR (400 MHz, DMSO-d₆) δ=10.98 (s, 1H), 8.31 (d, J=8.4 Hz, 1H), 8.22 (s, 1H), 8.17-8.12 (m, 1H), 7.88 (s, 1H), 7.53-7.47 (m, 2H), 7.46-7.40 (m, 1H), 7.31 (d, J=7.5 Hz, 1H), 7.16 (d, J=7.7 Hz, 1H), 5.12 (dd, J=5.1, 13.3 Hz, 1H), 4.91-4.83 (m, 1H), 4.49-4.35 (m, 2H), 4.34-4.26 (m, 1H), 4.09-3.88 (m, 7H), 3.31-3.29 (m, 3H), 3.18-3.01 (m, 5H), 2.97-2.86 (m, 1H), 2.73-2.54 (m, 11H), 2.04-1.95 (m, 1H), 1.93-1.77 (m, 2H), 1.55-1.33 (m, 2H), 1.24 (d, J=6.7 Hz, 6H).

### Example 134. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-((1-(2-(2,6-dioxopiperidin-3-yl)-1,3-dioxoisoindolin-5-yl)azetidin-3-yl)methyl)piperidin-4-yl)-3-methoxybenzamide (Compound 134)

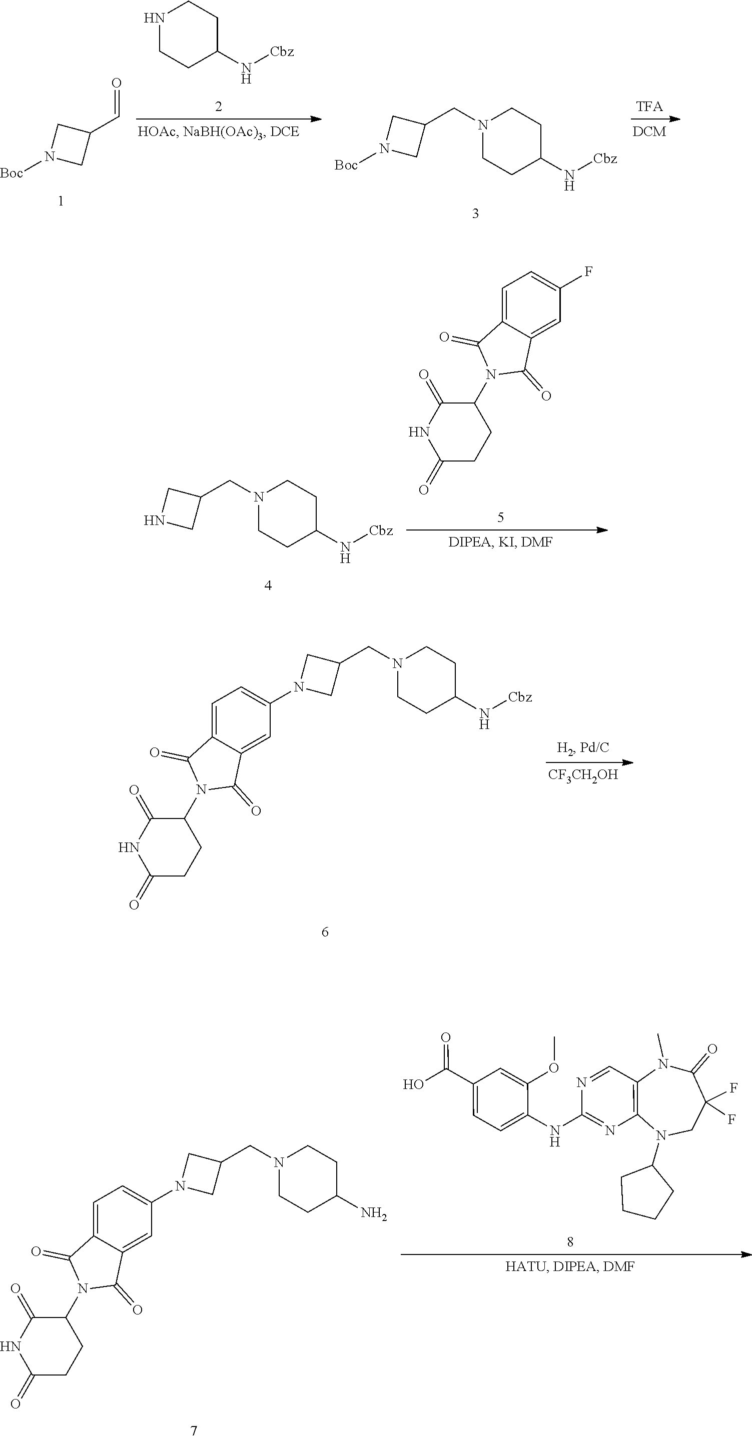

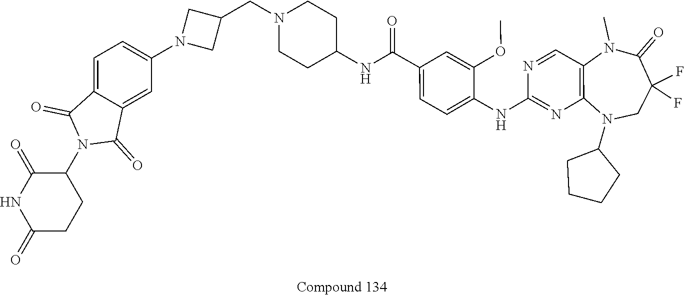 [SMILES: COc1cc(C(=O)NC2CCN(CC3CN(c4ccc5c(c4)C(=O)N(C4CCC(=O)NC4=O)C5=O)C3)CC2)ccc1Nc1ncc2c(n1)N(C1CCCC1)CC(F)(F)C(=O)N2C]

### Step 1. Synthesis of tert-butyl 3-((4-(((benzyloxy)carbonyl)amino)piperidin-1-yl)methyl)azetidine-1-carboxylate (3)

To a mixture of tert-butyl 3-formylazetidine-1-carboxylate (2.0 g, 10.80 mmol), benzyl piperidin-4-ylcarbamate (2.53 g, 10.80 mmol) and HOAc (1.95 g, 32.39 mmol, 1.85 mL) in DCE (20 mL) was added NaBH(OAc)₃ (6.87 g, 32.39 mmol). The mixture was stirred at 25° C. for 12 h. LCMS showed a main peak with desired mass. The reaction mixture was diluted with water (100 mL) and extracted with EtOAc (100 mL×3). The combined organic layers were washed with saturated sodium bicarbonate solution (50 mL) and then brine (50 mL), dried over anhydrous sodium sulfate, filtered and concentrated under reduced pressure to give a residue. The residue was purified by flash silica gel chromatography (ISCO®; 20 g Sepa Flash® Silica Flash Column, eluent of 0˜30% petroleum ether:EtOAc/methanol (v/v=1/1) gradient @60 mL/min) to afford tert-butyl 3-((4-(((benzyloxy)carbonyl)amino)piperidin-1-yl)methyl)azetidine-1-carboxylate (4.8 g, crude) as yellow oil. MS(M+H)⁺=404.3

### Step 2. Synthesis of benzyl (1-(azetidin-3-ylmethyl)piperidin-4-yl)carbamate (4)

To a solution of tert-butyl 3-((4-(((benzyloxy)carbonyl)amino)piperidin-1-yl)methyl)azetidine-1-carboxylate (0.5 g. 1.24 mmol) in DCM (4 mL) was added TFA (141.29 mg. 1.24 mmol. 91.75 μL). The mixture was stirred at 25° C. for 1 h. TLC (methanol:dichloromethane=10:1) showed the starting material was consumed completely. The reaction mixture was concentrated under reduced pressure to afford benzyl (1-(azetidin-3-ylmethyl)piperidin-4-yl)carbamate (520 mg, crude, TFA) as yellow oil, which was used for the next step directly. MS(M+H)⁺=303.9

### Step 3. Synthesis of benzyl (1-((1-(2-(2,6-dioxopiperidin-3-yl)-1,3-dioxoisoindolin-5-yl)azetidin-3-yl)methyl)piperidin-4-yl)carbamate (6)

To a solution of benzyl (1-(azetidin-3-ylmethyl)piperidin-4-yl)carbamate (0.5 g, 1.20 mmol, TFA) in DMF (8 mL) were added KI (59.65 mg, 359.35 μmol), 2-(2,6-dioxopiperidin-3-yl)-5-fluoroisoindoline-1,3-dione (330.86 mg, 1.20 mmol) and DIPEA (464.43 mg, 3.59 mmol, 625.92 μL) at 25° C. The mixture was stirred at 90° C. for 12 h. LCMS showed the desired mass. The reaction mixture was diluted with water (50 mL) and extracted with EtOAc (20 mL×4). The combined organic layers were washed with saturated brine (20 mL), dried over anhydrous sodium sulfate, filtered and concentrated under reduced pressure to give a residue. The residue was purified by flash silica gel chromatography (ISCO®; 20 g Sepa Flash® Silica Flash Column, eluent of 0˜50% petroleum ether/EtOAc:ethanol (v/v=1:1) gradient @60 mL/min) and then by prep-HPLC (column: Phenomenex luna C18 150×40 mm×15 μm; mobile phase: [water(FA)-ACN]; B %: 10%-40%, 10 min) followed by lyophilization to afford benzyl (1-((1-(2-(2,6-dioxopiperidin-3-yl)-1,3-dioxoisoindolin-5-yl)azetidin-3-yl)methyl)piperidin-4-yl)carbamate (120 mg, 213.15 μmol, 17.79% yield, 99.4% purity) as a yellow solid. MS(M+H)⁺=560.3

### Step 4. Synthesis of 5-(3-((4-aminopiperidin-1-yl)methyl)azetidin-1-yl)-2-(2,6-dioxopiperidin-3-yl) isoindoline-1,3-dione (7)

To a mixture of Pd/C (30 mg, 10% purity) in CF₃CH₂OH (5 mL) was added benzyl (1-((1-(2-(2,6-dioxopiperidin-3-yl)-1,3-dioxoisoindolin-5-yl)azetidin-3-yl)methyl)piperidin-4-yl)carbamate (120 mg, 214.43 μmol) at 25° C. The mixture was stirred at 25° C. for 12 h under H₂ atmosphere (15 Psi). TLC (dichloromethane:methanol=10:1) showed the starting material was consumed completely and one new spot was formed. The mixture was filtered to remove the catalyst. The filtrate was concentrated under reduced pressure to afford 5-(3-((4-aminopiperidin-1-yl)methyl)azetidin-1-yl)-2-(2,6-dioxopiperidin-3-yl)isoindoline-1,3-dione (90 mg, crude) as yellow oil, which was used directly without further purification. MS(M+H)⁺=426.0

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-((1-(2-(2,6-dioxopiperidin-3-yl)-1,3-dioxoisoindolin-5-yl)azetidin-3-yl)methyl)piperidin-4-yl)-3-methoxybenzamide (Compound 134)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (80 mg, 178.80 μmol) in DMF (2 mL) were added HATU (135.97 mg, 357.59 μmol) and DIPEA (69.32 mg, 536.39 μmol, 93.43 μL). Then 5-(3-((4-aminopiperidin-1-yl)methyl)azetidin-1-yl)-2-(2,6-dioxopiperidin-3-yl)isoindoline-1,3-dione (76.07 mg, 178.80 μmol) was added to the mixture after 0.5 h. The mixture was stirred at 25° C. for 12 h. LCMS showed the desired mass. The reaction mixture was diluted with water (20 mL) and extracted with EtOAc (10 mL×3). The combined organic layers were washed with saturated brine (10 mL), dried over anhydrous sodium sulfate, filtered and concentrated under reduced pressure to give a residue. The residue was purified by prep-HPLC (column: Phenomenex luna C18 150×40 mm×15 μm; mobile phase: [water(FA)-ACN]; B %: 18%-48%, 10 min) and then by prep-HPLC (column: Waters Xbridge 150×25 mm×5 μm; mobile phase: [water(NH₄HCO₃)-ACN]; B %: 38%-68%, 8 min) followed by lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-((1-(2-(2,6-dioxopiperidin-3-yl)-1,3-dioxoisoindolin-5-yl)azetidin-3-yl)methyl)piperidin-4-yl)-3-methoxybenzamide (21 mg, 24.34 μmol, 13.61% yield, 99.1% purity) as a yellow solid. MS(M+H)⁺=855.7.

¹H NMR (400 MHz, DMSO-d₆) δ=11.08 (s, 1H), 8.32-8.24 (m, 2H), 8.22-8.10 (m, 1H), 7.97 (s, 1H), 7.64 (d, J=8.1 Hz, 1H), 7.54-7.46 (m, 2H), 6.78 (s, 1H), 6.64 (d, J=8.6 Hz, 1H), 5.05 (dd, J=5.1, 12.8 Hz, 1H), 4.82-4.71 (m, 1H), 4.18-4.10 (m, 2H), 4.05 (t, J=14.0 Hz, 2H), 3.94 (s, 3H), 3.90-3.66 (m, 3H), 3.33 (s, 3H), 3.14-2.82 (m, 4H), 2.62-2.55 (m, 5H), 2.07-1.90 (m, 4H), 1.86-1.54 (m, 10H).

### Example 135. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-((1-(2-(2,6-dioxopiperidin-3-yl)-1,3-dioxoisoindolin-4-yl)azetidin-3-yl)methyl)piperidin-4-yl)-3-methoxybenzamide (Compound 135)

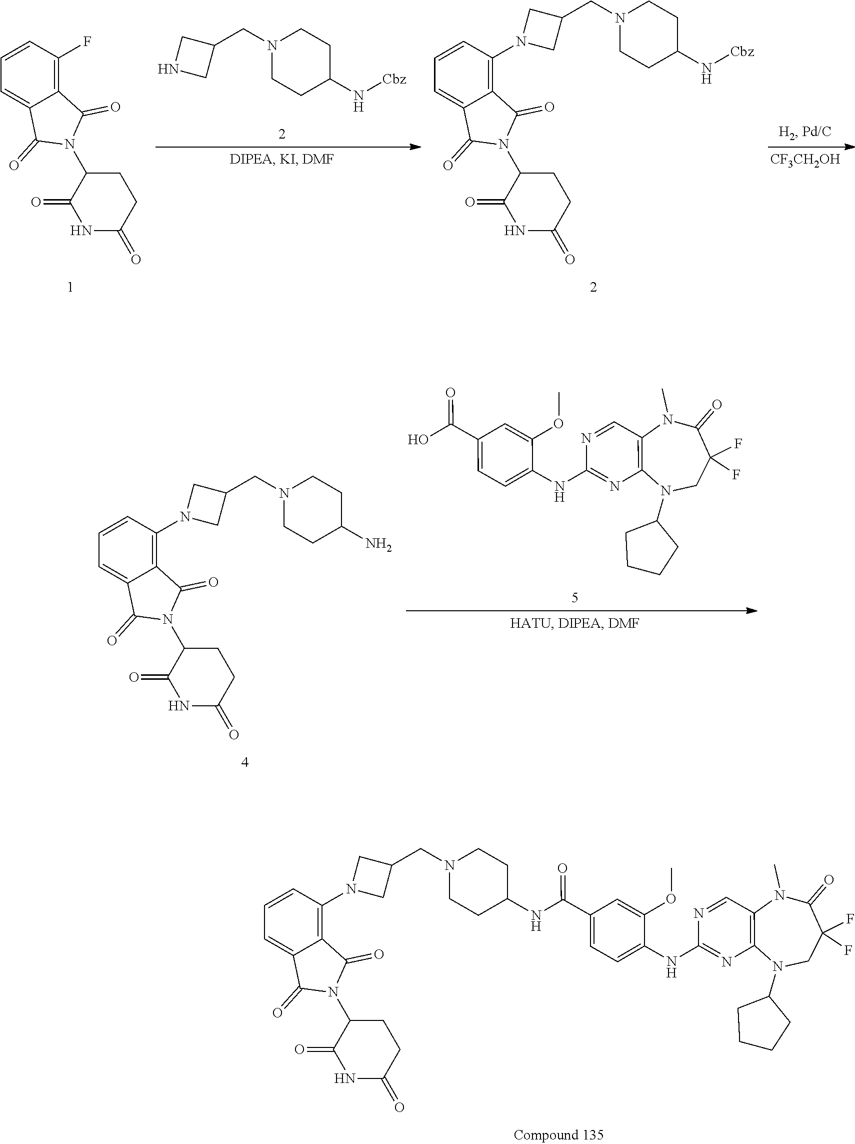

The compound 135 was synthesized by the method described in the scheme similar to the method described in Example 134.

MS(M+H)⁺=855.3, ¹H NMR (400 MHz, DMSO-d₆) δ=11.07 (s, 1H), 8.32-8.25 (m, 2H), 8.13-8.11 (m, 1H), 7.96 (s, 1H), 7.59-7.53 (m, 1H), 7.51-7.46 (m, 2H), 7.11 (d, J=7.0 Hz, 1H), 6.78 (d, J=8.6 Hz, 1H), 5.05 (dd, J=5.3, 12.7 Hz, 1H), 4.78-4.74 (m, 1H), 4.34-4.25 (m, 2H), 4.04 (t, J=14.1 Hz, 2H), 3.94 (s, 3H), 3.92-3.69 (m, 4H), 3.32-3.27 (m, 3H), 2.98-2.80 (m, 4H), 2.62-2.53 (m, 4H), 2.05-1.91 (m, 4H), 1.86-1.76 (m, 2H), 1.74-1.68 (m, 2H), 1.64-1.58 (m, 6H).

### Example 136. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(2-(2,6-dioxopiperidin-3-yl)-1,3-dioxoisoindolin-4-yl)piperidin-4-yl)methyl)-7-azaspiro[3.5]nonan-2-yl)-3-methoxybenzamide (Compound 136)

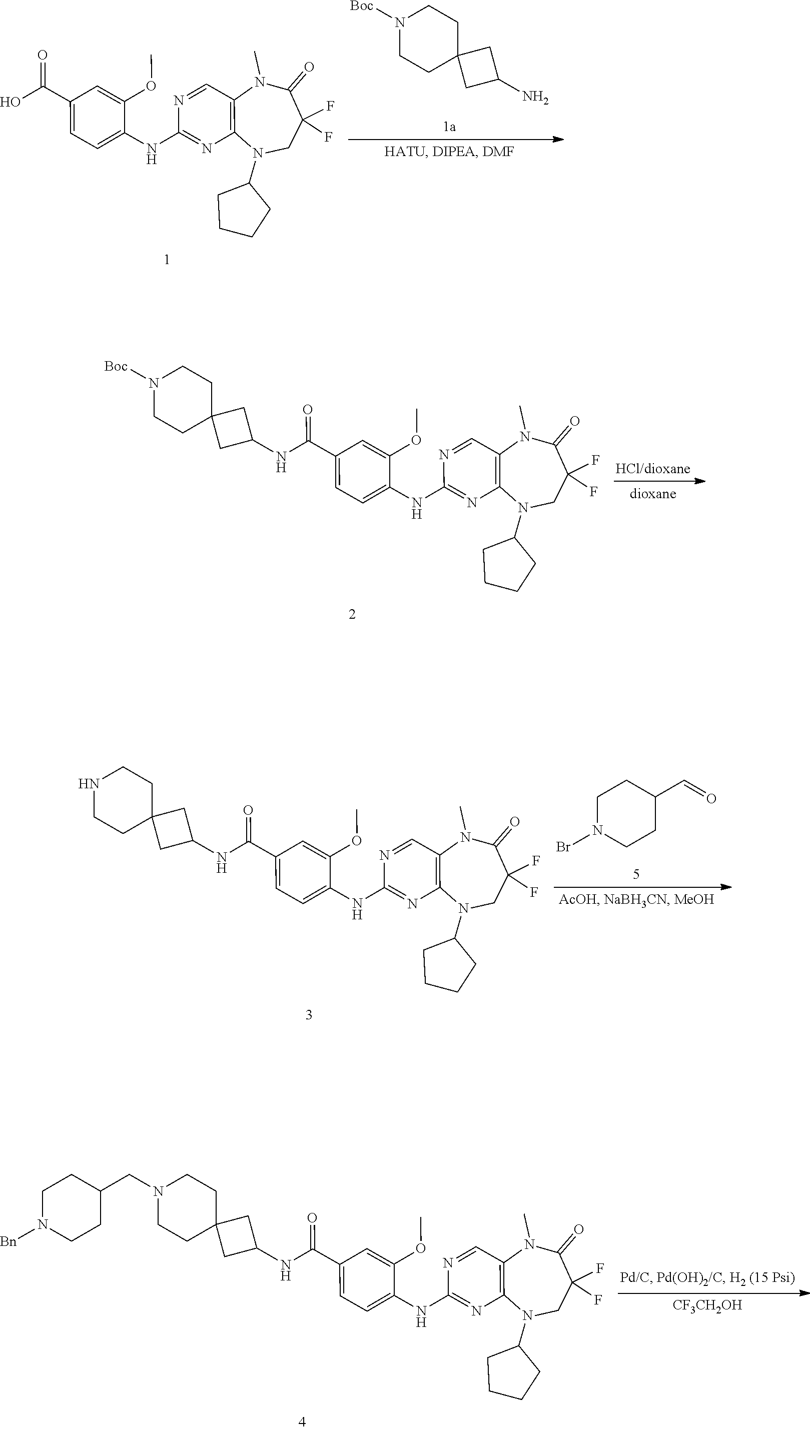

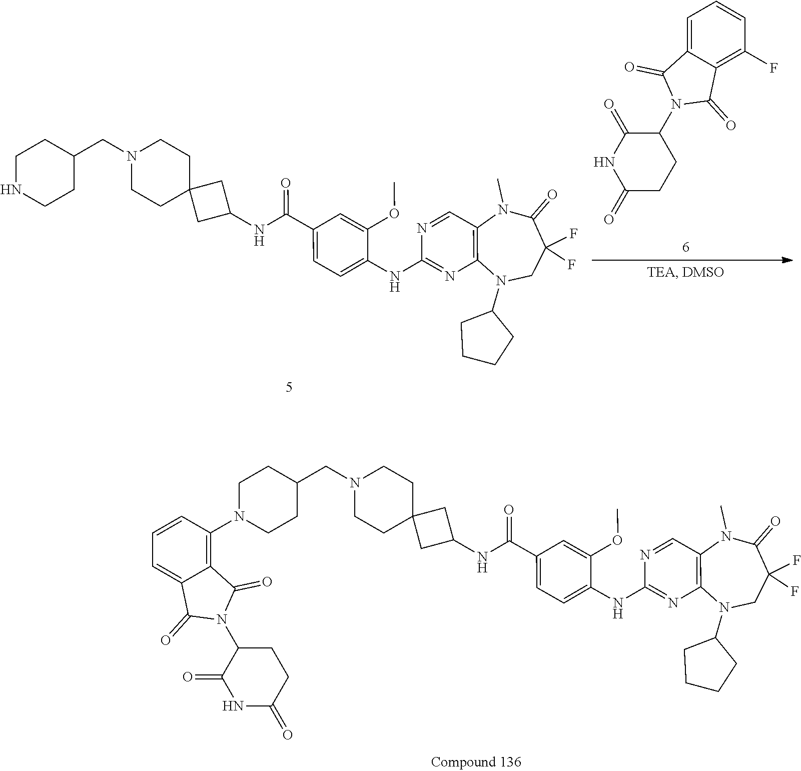

### Step 1. Synthesis of tert-butyl 2-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)-7-azaspiro[3.5]nonane-7-carboxylate (2)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (796 mg, 1.78 mmol) in DMF (5 mL) was added HATU (811.73 mg, 2.13 mmol) and DIPEA (689.77 mg, 5.34 mmol, 929.60 μL). The mixture was stirred at 25° C. for 10 min. To the mixture was added tert-butyl 2-amino-7-azaspiro[3.5]nonane-7-carboxylate (427.57 mg, 1.78 mmol). The resulting mixture was stirred at 25° C. for 2 h. LCMS showed the starting material was consumed completely and a main peak with desired mass. The mixture was poured into water (30 mL) and extracted with EtOAc (10 mL×3). The combined organic phase was washed with brine (10 mL×3), dried over Na₂SO₄, filtered and concentrated under reduced pressure to give a residue. The residue was purified by flash silica gel chromatography (25 g SepaFlash® Silica Flash Column, Eluent of 0˜100% EtOAc/Petroleum ether gradient @70 mL/min) to afford tert-butyl 2-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)-7-azaspiro[3.5]nonane-7-carboxylate (1.2 g, crude) as brown oil, which was used for the next step directly. MS(M+H)⁺=670.4

### Step 2. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(7-azaspiro[3.5]nonan-2-yl)benzamide (3)

To a solution of tert-butyl 2-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)-7-azaspiro[3.5]nonane-7-carboxylate (1.2 g, 1.79 mmol) in dioxane (5 mL) was added HCl/dioxane (4 M, 30 mL) at 25° C. The resulting mixture was stirred at 25° C. for 0.5 hr. LCMS showed the starting material was consumed completely and a main peak with desired mass. The mixture solution was concentrated to afford 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(7-azaspiro[3.5]nonan-2-yl)benzamide (1.5 g, crude, HCl) as brown solid, which was used for the next step directly. MS(M+H)⁺=570.3

### Step 3. Synthesis of N-(7-((1-benzylpiperidin-4-yl)methyl)-7-azaspiro[3.5]nonan-2-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (4)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(7-azaspiro[3.5]nonan-2-yl)benzamide (1.3 g, 2.28 mmol), 1-benzylpiperidine-4-carbaldehyde (463.91 mg, 2.28 mmol) in MeOH (5 mL) were added HOAc (274.08 mg, 4.56 mmol, 261.03 μL). The mixture was stirred at 25° C. for 10 min. To the reaction mixture was added NaBH₃CN (286.83 mg, 4.56 mmol) at 25° C. The mixture was stirred at 25° C. for 12 h. LCMS showed the starting material was consumed completely and a main peak with desired mass. The reaction solution was concentrated to remove the organic phase. The crude product was dissolved with DCM (50 mL) washed with saturated NaHCO₃ (30 mL), dried over Na₂SO₄, filtered and concentrated under reduced pressure to give a residue. The residue was purified by flash silica gel chromatography (10 g SepaFlash® Silica Flash Column, Eluent of 0˜100% EtOAc/Petroleum ether gradient @70 mL/min; Eluent of 0˜50% Methanol/EtOAc @70 mL/min) to afford N-(7-((1-benzylpiperidin-4-yl)methyl)-7-azaspiro[3.5]nonan-2-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (1 g, 1.28 mmol, 56.15% yield, 97% purity) as white solid, which was used for the next step directly. MS(M+H)⁺=757.3

### Step 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(7-(piperidin-4-ylmethyl)-7-azaspiro[3.5]nonan-2-yl)benzamide (5)

To a solution of N-(7-((1-benzylpiperidin-4-yl)methyl)-7-azaspiro[3.5]nonan-2-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (0.2 g, 264.23 μmol) in CF₃CH₂OH (10 mL) was added Pd/C (0.2 g, 264.23 μmol, 10% purity). The mixture was stirred at 25° C. under H₂ (15 Psi) for 12 hr. LCMS showed most of the starting material was still remained. Another portion of Pd/C (0.1 g, 264.23 μmol, 10% purity) and Pd(OH)₂/C (0.1 g, 264.23 μmol, 20% purity) was added to the reaction mixture at 25° C. The resulting mixture was stirred at 60° C. under H₂ (15 Psi) for 12 hr. LCMS showed the starting material was consumed completely, and a peak (53%) with desired mass. The reaction mixture was filtered through a celite pad and the filtrate was concentrated to afford 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(7-(piperidin-4-ylmethyl)-7-azaspiro[3.5]nonan-2-yl)benzamide (176 mg, crude) as colorless oil, which was used for the next step directly. MS(M+H)⁺=667.4

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(2-(2,6-dioxopiperidin-3-yl)-1,3-dioxoisoindolin-4-yl)piperidin-4-yl)methyl)-7-azaspiro[3.5]nonan-2-yl)-3-methoxybenzamide (Compound 136)

A solution of 2-(2,6-dioxopiperidin-3-yl)-4-fluoroisoindoline-1,3-dione (32.81 mg, 118.78 μmol), 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(7-(piperidin-4-ylmethyl)-7-azaspiro[3.5]nonan-2-yl)benzamide (88 mg, 131.97 μmol) and TEA (40.06 mg, 395.92 μmol, 55.11 μL) in DMSO (2 mL) was stirred at 90° C. for 4 hr. LCMS showed the starting material was consumed completely and the desired mass. The mixture was poured into water (30 mL) and extracted with EtOAc (20 mL×3). The combined organic phase was washed with brine (10 mL×3), dried over Na₂SO₄, filtered and concentrated under reduced pressure to give a residue. The residue was purified by prep-TLC (Dichloromethane Methanol=10:1; R_{f}=0.4). The crude product was purified by prep-HPLC (column: Waters Xbridge 150×25 mm×5 μm; mobile phase: [water(NH₄HCO₃)-ACN]; B %: 47%-77%, 8 min) and lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(2-(2,6-dioxopiperidin-3-yl)-1,3-dioxoisoindolin-4-yl)piperidin-4-yl)methyl)-7-azaspiro[3.5]nonan-2-yl)-3-methoxybenzamide (26.1 mg, 27.99 μmol, 21.21% yield, 99% purity) as yellow solid. MS(M+H)⁺=923.4

¹H NMR (400 MHz, DMSO-d₆) δ=11.08 (s, 1H), 8.43 (br d, J=7.2 Hz, 1H), 8.32-8.20 (m, 2H), 7.96 (s, 1H), 7.72-7.60 (m, 1H), 7.55-7.41 (m, 2H), 7.38-7.22 (m, 2H), 5.08 (dd, J=5.4, 12.7 Hz, 1H), 4.82-4.70 (m, 1H), 4.46-4.33 (m, 1H), 4.11-3.98 (m, 2H), 3.94 (s, 3H), 3.68 (br d, J=10.4 Hz, 2H), 3.33-3.32 (m, 3H), 2.95-2.75 (m, 3H), 2.64-2.54 (m, 2H), 2.35-2.08 (m, 8H), 2.07-1.89 (m, 3H), 1.85-1.51 (m, 15H), 1.38-1.21 (m, 2H)

### Example 137. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(7-((1-(2-(2,6-dioxopiperidin-3-yl)-1,3-dioxoisoindolin-5-yl)piperidin-4-yl)methyl)-7-azaspiro[3.5]nonan-2-yl)-3-methoxybenzamide (Compound 137)

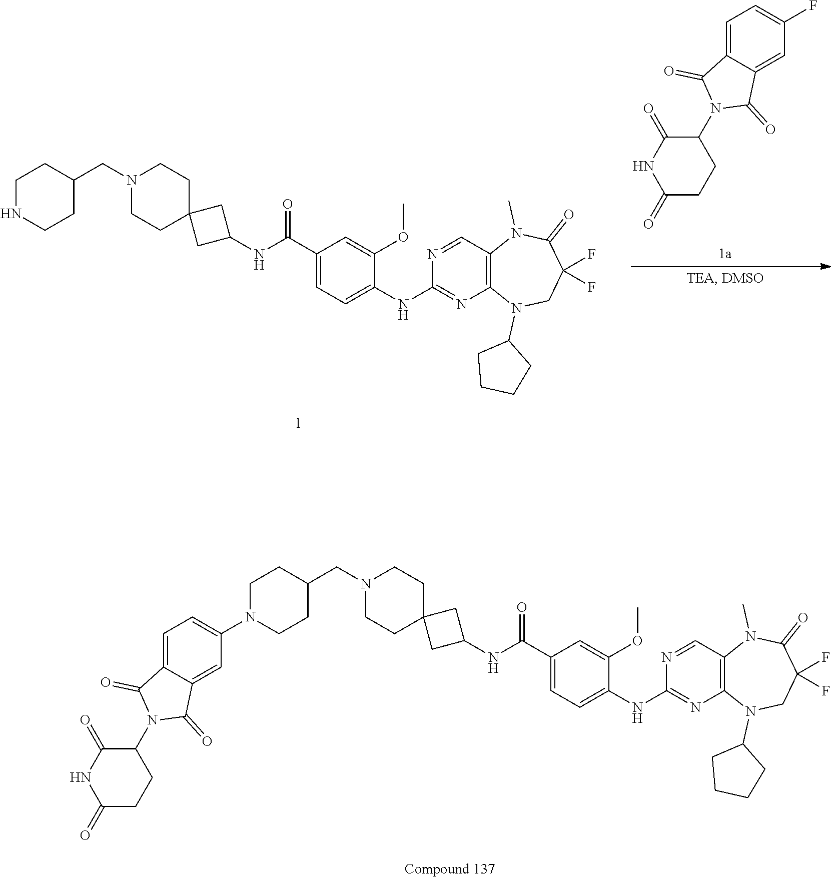 [SMILES: COc1cc(C(=O)NC2CC3(CCN(CC4CCNCC4)CC3)C2)ccc1Nc1ncc2c(n1)N(C1CCCC1)CC(F)(F)C(=O)N2C ; O=C1CCC(N2C(=O)c3ccc(F)cc3C2=O)C(=O)N1 ; COc1cc(C(=O)NC2CC3(CCN(CC4CCN(c5ccc6c(c5)C(=O)N(C5CCC(=O)NC5=O)C6=O)CC4)CC3)C2)ccc1Nc1ncc2c(n1)N(C1CCCC1)CC(F)(F)C(=O)N2C]

The compound 137 was synthesized by the method described in the scheme similar to the method described in Example 136.

MS (M+H)⁺=923.4, ¹H NMR (400 MHz, DMSO-d₆) δ=11.07 (s, 1H), 8.42 (br d, J=7.4 Hz, 1H), 8.30-8.23 (m, 2H), 7.96 (s, 1H), 7.64 (d, J=8.5 Hz, 1H), 7.52-7.45 (m, 2H), 7.30 (s, 1H), 7.22 (dd, J=1.7, 8.6 Hz, 1H), 5.06 (dd, J=5.4, 12.9 Hz, 1H), 4.76 (quin, J=8.1 Hz, 1H), 4.45-4.32 (m, 1H), 4.11-3.97 (m, 4H), 3.94 (s, 3H), 3.30 (br s, 3H), 3.02-2.82 (m, 3H), 2.64-2.53 (m, 2H), 2.34-2.08 (m, 8H), 2.04-1.91 (m, 3H), 1.84-1.53 (m, 15H), 1.23-1.06 (m, 2H).

### Example 138. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-((1-(2-(2,6-dioxopiperidin-3-yl)-1,3-dioxoisoindolin-4-yl)piperidin-4-yl)methyl)azetidin-3-yl)-3-methoxybenzamide (Compound 138)

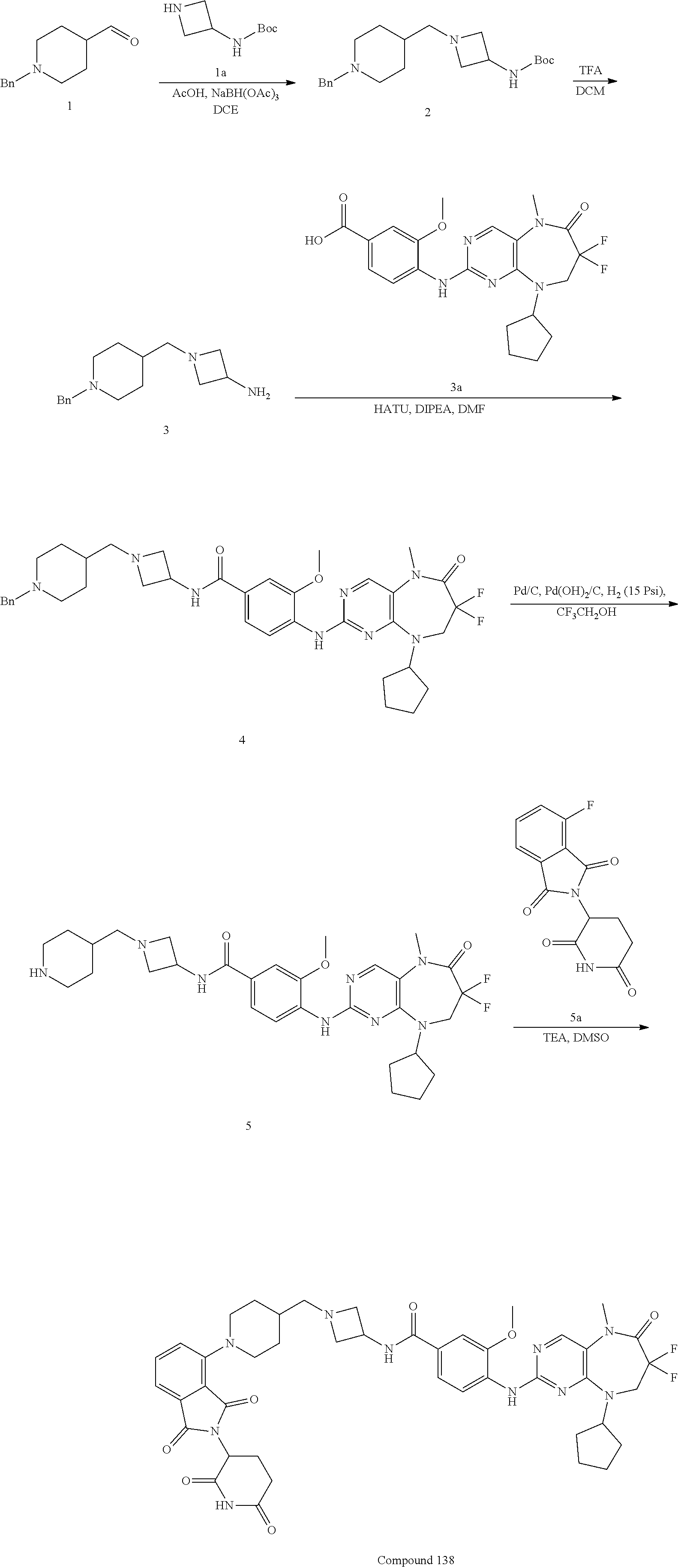

### Step 1. Synthesis of tert-butyl (1-((1-benzylpiperidin-4-yl)methyl)azetidin-3-yl)carbamate (2)

To a solution of 1-benzylpiperidine-4-carbaldehyde (0.6 g, 2.95 mmol), tert-butyl azetidin-3-ylcarbamate (508.34 mg, 2.95 mmol) in DCE (30 mL) were added AcOH (177.24 mg, 2.95 mmol, 168.80 μL). The mixture was stirred at 25° C. for 10 min. To the mixture was added NaBH(OAc)₃ (1.25 g, 5.90 mmol) at 25° C. The resulting mixture was stirred at 25° C. for 12 h. TLC (dichloromethane:methanol=10:1; R_{f}=0) showed the starting material was consumed completely and new spot was formed. The mixture was diluted with saturated NaHCO₃ (20 mL) and extracted with DCM (30 mL×3). The combined organic layer was dried over Na₂SO₄ and concentrated. The residue was purified by flash silica gel chromatography (12 g SepaFlash® Silica Flash Column, Eluent of 0˜100% EtOAc/Petroleum ether gradient @60 mL/min; Eluent of 0˜50% Methanol/EtOAc @60 mL/min) to afford tert-butyl (1-((1-benzylpiperidin-4-yl)methyl)azetidin-3-yl)carbamate (1.1 g, crude) as yellow oil, which was used for the next step directly. MS(M+H)⁺=360.2

### Step 2. Synthesis of 1-((1-benzylpiperidin-4-yl)methyl)azetidin-3-amine (3)

To a solution of tert-butyl (1-((1-benzylpiperidin-4-yl)methyl)azetidin-3-yl)carbamate (450 mg, 1.25 mmol) in DCM (10 mL) was added TFA (3.08 g, 27.01 mmol, 2 mL) at 25° C. The resulting mixture was stirred at 25° C. for 0.5 hr. LCMS showed the starting material was consumed completely and a main peak with desired mass. The solution was concentrated under reduced pressure to afford 1-((1-benzylpiperidin-4-yl)methyl)azetidin-3-amine (460 mg, crude, TFA) as yellow oil, which was used for the next step directly. MS(M+H)⁺=260.2

### Step 3. Synthesis of N-(1-((1-benzylpiperidin-4-yl)methyl)azetidin-3-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (4)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (450.95 mg, 1.01 mmol) in DMF (2 mL) was added HATU (459.86 mg, 1.21 mmol) and DIPEA (390.77 mg, 3.02 mmol, 526.64 μL). The mixture was stirred at 25° C. for 10 min. To the mixture was added 1-((1-benzylpiperidin-4-yl)methyl)azetidin-3-amine (451.62 mg, 1.21 mmol, TFA). The resulting mixture was stirred at 25° C. for 1 h. LCMS showed the starting material was consumed completely and the desired mass. The mixture was poured into water (40 mL) and extracted with EtOAc (20 mL×4). The combined organic layers were dried over Na₂SO₄ and concentrated to give the crude product. The crude product was purified by prep-HPLC (column: Phenomenex luna C18 150×40 mm×15 μm; mobile phase: [water(FA)-ACN]; B %: 10%-40%, 10 min) and lyophilized to afford N-(1-((1-benzylpiperidin-4-yl)methyl)azetidin-3-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (0.2 g, 287.45 μmol, 28.52% yield, 99% purity) as yellow oil, which was used for the next step directly. MS(M+H)⁺=689.5

### Step 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(1-(piperidin-4-ylmethyl)azetidin-3-yl) benzamide (5)

To a solution of N-(1-((1-benzylpiperidin-4-yl)methyl)azetidin-3-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (200 mg, 290.36 μmol) in CF₃CH₂OH (15 mL) was added Pd/C (100 mg, 10% purity) and Pd(OH)₂/C (100 mg, 20% purity) at 25° C. The mixture was stirred at 50° C. under H₂ (15 Psi) for 12 hr. LCMS showed the starting material was consumed completely and a main peak with desired mass. The reaction mixture was filtered through a celite pad and the filtrate was concentrated to afford 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(1-(piperidin-4-ylmethyl)azetidin-3-yl)benzamide (170 mg, crude) as yellow oil, which was used for the next step directly. MS(M+H)⁺=599.1

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-((1-(2-(2,6-dioxopiperidin-3-yl)-1,3-dioxoisoindolin-4-yl)piperidin-4-yl)methyl)azetidin-3-yl)-3-methoxybenzamide (Compound 138)

A solution of 2-(2,6-dioxopiperidin-3-yl)-4-fluoroisoindoline-1,3-dione (33.33 mg, 120.68 μmol), 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(1-(piperidin-4-ylmethyl)azetidin-3-yl)benzamide (85 mg, 141.98 μmol) and TEA (43.10 mg, 425.93 μmol, 59.28 μL) in DMSO (2 mL) was stirred at 90° C. for 3 hr. LCMS showed the starting material was consumed completely and a main peak with desired mass. The mixture solution was poured into water (20 mL) and extracted with EtOAc (10 mL×4). The combined organic layers were dried over Na₂SO₄ and concentrated to give the crude product. The residue was purified by prep-TLC (dichloromethane:Methanol=10:1; R_{f}=0.7) to give the crude product. The crude product was purified by prep-HPLC (column: Waters Xbridge 150×25 mm×5 μm; mobile phase: [water(NH₄HCO₃)-ACN]; B %: 44%-74%, 8 min) and lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-((1-(2-(2,6-dioxopiperidin-3-yl)-1,3-dioxoisoindolin-4-yl)piperidin-4-yl)methyl)azetidin-3-yl)-3-methoxybenzamide (22.4 mg, 25.94 μmol, 18.27% yield, 99% purity) as yellow solid. MS(M+H)⁺=855.5

¹H NMR (400 MHz, CDCl₃) δ=8.50 (d, J=8.5 Hz, 1H), 8.42-8.20 (m, 1H), 8.07 (s, 1H), 7.77 (s, 1H), 7.57 (dd, J=7.3, 8.3 Hz, 1H), 7.47 (d, J=1.4 Hz, 1H), 7.40-7.31 (m, 2H), 7.17 (d, J=8.5 Hz, 1H), 6.88-6.61 (m, 1H), 4.96 (dd, J=5.3, 12.2 Hz, 1H), 4.90-4.73 (m, 2H), 3.99 (s, 3H), 3.90 (t, J=13.5 Hz, 2H), 3.75 (br t, J=6.0 Hz, 4H), 3.42 (s, 3H), 3.21 (br s, 2H), 2.94-2.67 (m, 5H), 2.50 (br d, J=5.1 Hz, 2H), 2.17-2.04 (m, 3H), 1.87 (br d, J=11.3 Hz, 2H), 1.82-1.63 (m, 9H).

### Example 139. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-((1-(2-(2,6-dioxopiperidin-3-yl)-1,3-dioxoisoindolin-5-yl)piperidin-4-yl)methyl)azetidin-3-yl)-3-methoxybenzamide (Compound 139)

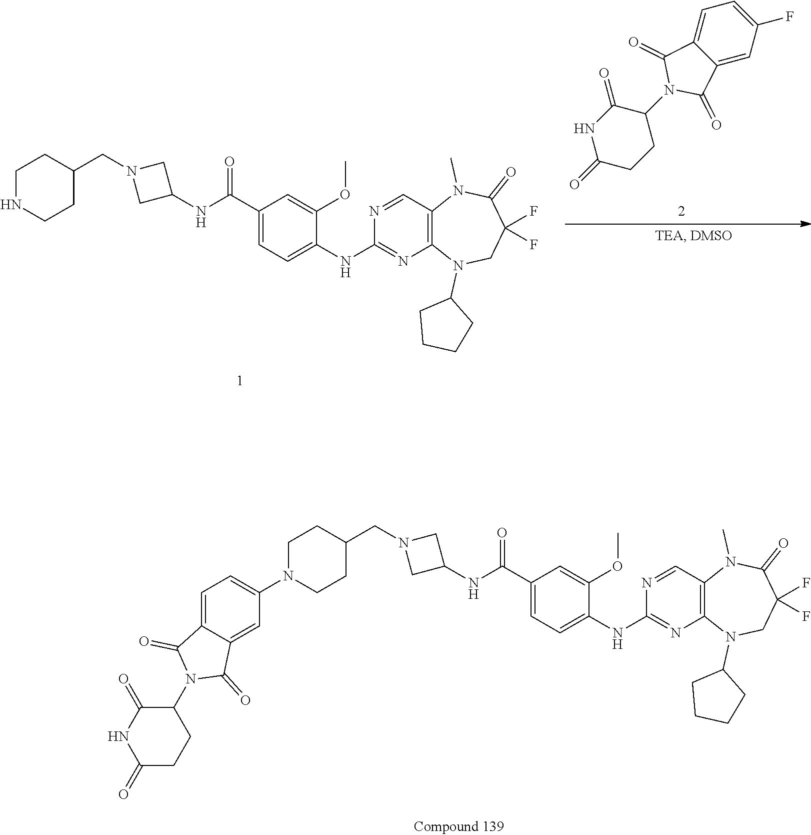 [SMILES: COc1cc(C(=O)NC2CN(CC3CCNCC3)C2)ccc1Nc1ncc2c(n1)N(C1CCCC1)CC(F)(F)C(=O)N2C ; O=C1CCC(N2C(=O)c3ccc(F)cc3C2=O)C(=O)N1 ; COc1cc(C(=O)NC2CN(CC3CCN(c4ccc5c(c4)C(=O)N(C4CCC(=O)NC4=O)C5=O)CC3)C2)ccc1Nc1ncc2c(n1)N(C1CCCC1)CC(F)(F)C(=O)N2C]

The compound 139 was synthesized by the method described in the scheme similar to the method described in Example 138.

MS(M+H)⁺=855.5, ¹H NMR (400 MHz, DMSO-d₆) δ=11.07 (s, 1H), 8.62 (d, J=6.8 Hz, 1H), 8.32-8.22 (m, 2H), 7.97 (s, 1H), 7.65 (d, J=8.5 Hz, 1H), 7.54-7.46 (m, 2H), 7.30 (d, J=1.8 Hz, 1H), 7.22 (dd, J=2.1, 8.7 Hz, 1H), 5.06 (dd, J=5.4, 12.8 Hz, 1H), 4.83-4.71 (m, 1H), 4.47 (t, J=6.9 Hz, 1H), 4.13-3.98 (m, 4H), 3.94 (s, 3H), 3.58 (t, J=7.1 Hz, 2H), 3.30 (s, 3H), 3.05-2.81 (m, 5H), 2.63-2.52 (m, 2H), 2.36-2.32 (m, 2H), 2.04-1.88 (m, 3H), 1.83-1.67 (m, 4H), 1.66-1.54 (m, 5H), 1.26-1.10 (m, 2H).

### Example 140. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-((1-(2-(2,6-dioxopiperidin-3-yl)-1,3-dioxoisoindolin-4-yl)piperidin-4-yl)methyl)phenyl)-2-fluoro-5-methoxybenzamide (Compound 140)

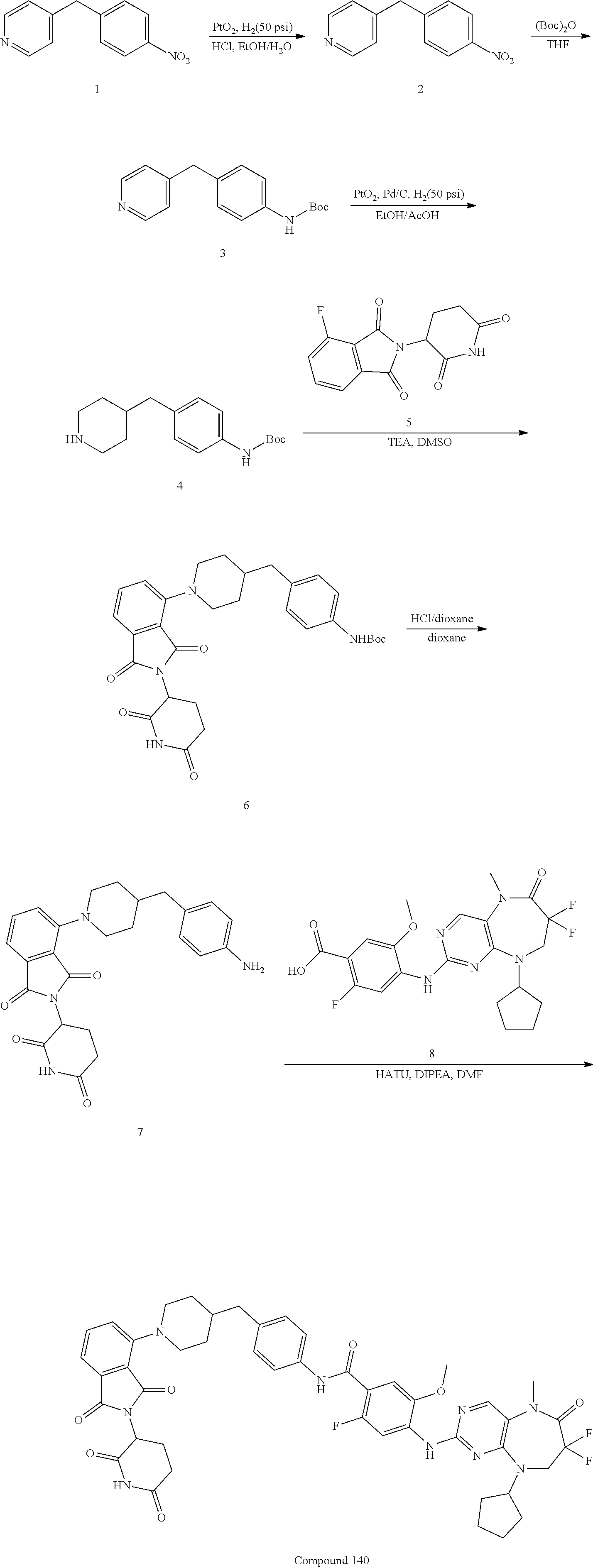

### Step 1. Synthesis of 4-(pyridin-4-ylmethyl)aniline (2)

To a solution of 4-(4-nitrobenzyl)pyridine (10 g, 46.68 mmol) in EtOH (120 mL), H₂O (3.89 mL) and HCl (12 M, 3.89 mL) was added PtO₂ (778.21 mg, 3.43 mmol) under N₂ atmosphere. The suspension was degassed and purged with H₂ for 3 times. The mixture was stirred under H₂ (50 Psi) at 20° C. for 16 h. TLC (SiO₂, Petroleum ether:EtOAc=10:1) indicated starting material remained and one new spot was detected. The reaction mixture was diluted with EtOH (300 mL) and filtered. The filtrate was concentrated in vacuum to afford 4-(4-pyridylmethyl) aniline (12 g, crude) as a yellow solid. MS(M+H)⁺=185.1

### Step 2. Synthesis of tert-butyl (4-(pyridin-4-ylmethyl)phenyl)carbamate (3)

To a solution of 4-(4-pyridylmethyl) aniline (5 g, 27.14 mmol) in THF (200 mL). Then a solution of (Boc)₂O (6.69 g, 30.67 mmol, 7.05 mL) with THF (30 mL) was added drop-wise at 0° C. and the resulting mixture was stirred at 20° C. for 16 h. LCMS showed starting material was consumed completely and a peak (87%) with desired mass. The reaction mixture was concentrated in vacuum. The residue was purified by flash silica gel chromatography (25 g SepaFlash® Silica Flash Column, Eluent of 0˜33% EtOAc/Petroleum ether gradient @100 mL/min) to afford tert-butyl (4-(pyridin-4-ylmethyl)phenyl)carbamate (4.4 g, 13.31 mmol, 49.03% yield, 86% purity) as a purple oil. MS(M+H)⁺=285.2

### Step 3. Synthesis of tert-butyl (4-(piperidin-4-ylmethyl)phenyl)carbamate (4)

To a solution of tert-butyl (4-(pyridin-4-ylmethyl)phenyl)carbamate (2 g, 7.03 mmol) in AcOH (4 mL) and EtOH (20 mL) were added Pd/C (200 mg, 10% purity) and PtO₂ (1.60 g, 7.03 mmol) under N₂ atmosphere. The suspension was degassed and purged with H₂ for 3 times. The mixture was stirred at 50° C. for 16 h under H₂ (50 Psi). LCMS showed starting material was consumed completely and a peak (63%) with desired mass. The reaction mixture was diluted with EtOH (300 mL) and filtered. The filtrate was concentrated in vacuum to afford tert-butyl (4-(piperidin-4-ylmethyl)phenyl)carbamate (2 g, crude) as an orange oil. MS(M+H)⁺=291.4

### Step 4. Synthesis of tert-butyl (4-((1-(2-(2,6-dioxopiperidin-3-yl)-1,3-dioxoisoindolin-4-yl)piperidin-4-yl)methyl) phenyl)carbamate (6)

To a solution of 2-(2,6-dioxo-3-piperidyl)-4-fluoro-isoindoline-1,3-dione (500 mg, 1.81 mmol) and tert-butyl (4-(piperidin-4-ylmethyl)phenyl)carbamate (735.93 mg, crude) in DMSO (8 mL) was added TEA (549.50 mg, 5.43 mmol, 755.85 μL) at 20° C. and the resulting mixture was stirred at 100° C. for 16 h. LCMS showed starting material was consumed completely and a peak (55%) with desired mass. The reaction mixture was diluted with H₂O (20 mL) and extracted with EtOAc (20 mL×3). The organic layer was washed with brine (20 mL×3), dried over Na₂SO₄, filtered and concentrated. The residue was purified by flash silica gel chromatography (12 g SepaFlash® Silica Flash Column, Eluent of 0˜50% EtOAc/Petroleum ether gradient @100 mL/min) to afford tert-butyl (4-((1-(2-(2,6-dioxopiperidin-3-yl)-1,3-dioxoisoindolin-4-yl)piperidin-4-yl)methyl)phenyl)carbamate (711 mg, 1.30 mmol, 71.86% yield) as a yellow solid. MS(M+H)⁺=547.0

### Step 5. Synthesis of 4-(4-(4-aminobenzyl)piperidin-1-yl)-2-(2,6-dioxopiperidin-3-yl)isoindoline-1,3-dione (7)

To a solution of tert-butyl (4-((1-(2-(2,6-dioxopiperidin-3-yl)-1,3-dioxoisoindolin-4-yl)piperidin-4-yl)methyl)phenyl)carbamate (200 mg, 365.89 μmol) in dioxane (3 mL) was added HCl/dioxane (4 M, 12 mL) at 20° C. and the resulting mixture was stirred at 20° C. for 0.5 h. LCMS showed starting material was consumed completely and a main peak (91%) with desired mass. The reaction mixture was concentrated in vacuum to afford 4-(4-(4-aminobenzyl)piperidin-1-yl)-2-(2,6-dioxopiperidin-3-yl)isoindoline-1,3-dione (177 mg, crude, HCl) as a yellow solid. MS(M+H)⁺=447.2

### Step 6. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-((1-(2-(2,6-dioxopiperidin-3-yl)-1,3-dioxoisoindolin-4-yl)piperidin-4-yl)methyl)phenyl)-2-fluoro-5-methoxybenzamide (Compound 140)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (80 mg, 171.89 μmol) in DMF (2 mL) were added HATU (71.89 mg, 189.07 μmol) and DIPEA (44.43 mg, 343.77 μmol, 59.88 μL). the mixture was stirred at 20° C. for 10 min, then a solution of 4-(4-(4-aminobenzyl)piperidin-1-yl)-2-(2,6-dioxopiperidin-3-yl)isoindoline-1,3-dione (99.62 mg, crude, HCl) and DIPEA (44.43 mg, 343.77 μmol, 59.88 μL) in DMF (2 mL) was added and the resulting mixture was stirred at 20° C. for 1 h. LCMS showed starting material was consumed completely and a peak (54%) with desired mass. The reaction mixture was diluted with H₂O (20 mL) and extracted with EtOAc (20 mL×3). The organic layer was washed with brine (20 mL×3), dried over Na₂SO₄, filtered and concentrated. The residue was purified by prep-HPLC (column: Unisil 3-100 C18 Ultra 150*50 mm*3 μm; mobile phase: [water(FA)-ACN]; B %: 65%-95%, 10 min), the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-((1-(2-(2,6-dioxopiperidin-3-yl)-1,3-dioxoisoindolin-4-yl)piperidin-4-yl)methyl)phenyl)-2-fluoro-5-methoxybenzamide (33 mg, 36.18 μmol, 21.05% yield, 98% purity) as a yellow solid. MS(M+H)⁺=894.1

¹H NMR (400 MHz, DMSO-d₆) δ=11.08 (s, 1H), 10.08 (s, 1H), 8.36-8.26 (m, 2H), 8.10 (s, 1H), 7.71-7.59 (m, 3H), 7.36-7.24 (m, 3H), 7.18 (d, J=8.4 Hz, 2H), 5.08 (dd, J=5.4, 12.8 Hz, 1H), 4.90-4.78 (m, 1H), 4.14-4.02 (m, 2H), 3.94 (s, 3H), 3.73-3.63 (m, 2H), 3.33-3.30 (m, 5H), 2.93-2.77 (m, 3H), 2.63-2.54 (m, 2H), 2.07-1.91 (m, 3H), 1.77-1.56 (m, 9H), 1.48-1.34 (m, 2H).

### Example 141. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(2-(2,6-dioxopiperidin-3-yl)-1,3-dioxoisoindolin-4-yl)piperazin-1-yl)ethoxy)phenyl)-2-fluoro-5-methoxybenzamide (Compound 141)

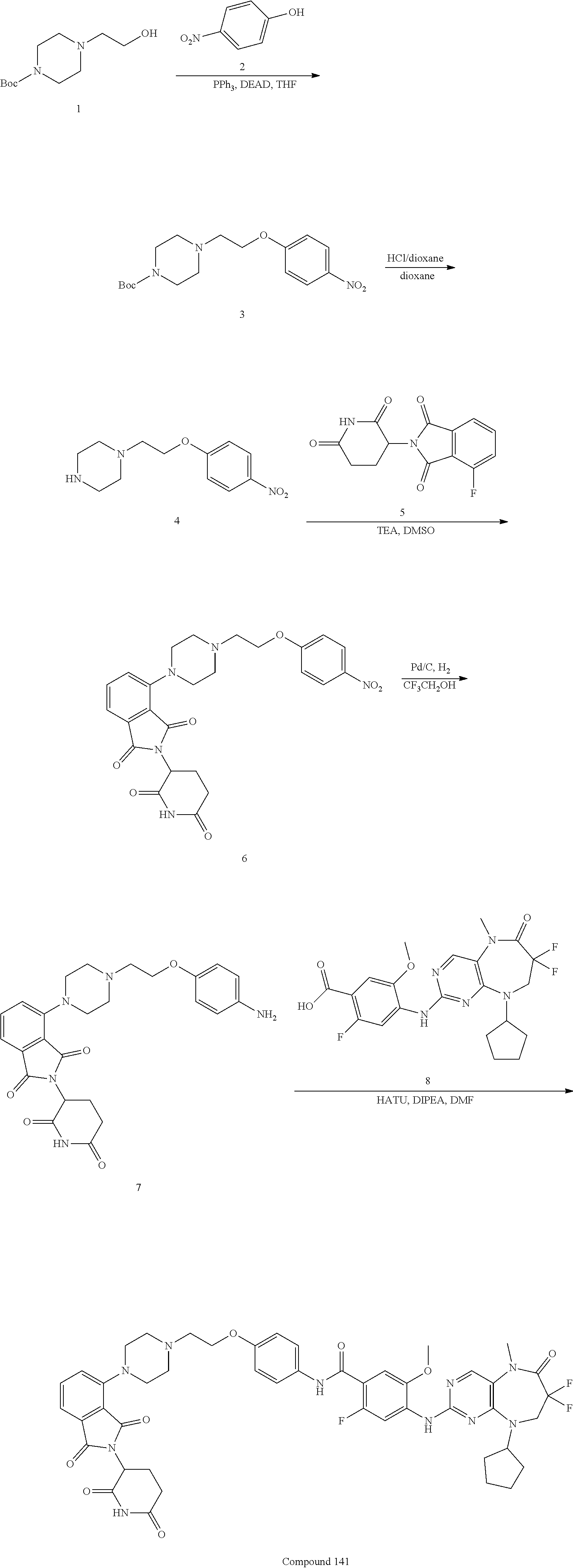

### Step 1. Synthesis of tert-butyl 4-(2-(4-nitrophenoxy)ethyl)piperazine-1-carboxylate (3)

To a mixture of tert-butyl 4-(2-hydroxyethyl) piperazine-1-carboxylate (2 g, 8.68 mmol) and 4-nitrophenol (1.45 g, 10.42 mmol), PPh₃ (3.42 g, 13.03 mmol) in THF (40 mL) was added DEAD (2.27 g, 13.03 mmol, 2.37 mL) drop-wise at 0° C. under N₂ and the resulting mixture was stirred at 20° C. for 16 h under N₂. LCMS showed starting material was consumed completely and a peak (27%) with desired mass. The reaction mixture was concentrated in vacuum. The residue was purified by flash silica gel chromatography (20 g SepaFlash® Silica Flash Column, Eluent of 0˜100% EtOAc/Petroleum ether gradient @100 mL/min) and re-purified by reversed-phase HPLC (method: 0.1% FA. Column I. D.95 mm*H₃₆₅ mm Welch Ultimate XB_C₁₈ 20-40 μm; 120 A Solvent for sample dissolution about 4.10 grams of sample dissolved in 45 mL of MeOH; Flow rate 200 mL/min; Mobile phase MeCN/H₂O; Gradient B % 5-35% 30 min; 35% 10 min Instrument Biotage Isolera One). The eluent was extracted with EtOAc (200 mL×3). The organic layer was dried over Na₂SO₄, filtered and concentrated to afford tert-butyl 4-(2-(4-nitrophenoxy)ethyl)piperazine-1-carboxylate (2.5 g, 7.04 mmol, 81.10% yield, 99% purity) as a yellow solid. MS(M+H)⁺=352.2

### Step 2. Synthesis of 1-(2-(4-nitrophenoxy)ethyl)piperazine (4)

To a solution of tert-butyl 4-(2-(4-nitrophenoxy)ethyl)piperazine-1-carboxylate (600 mg, 1.71 mmol) in dioxane (6 mL) was added HCl/dioxane (4 M, 12 mL) at 20° C. and the resulting mixture was stirred at 20° C. for 1 h. LCMS showed starting material was consumed completely and a main peak with desired mass. The reaction mixture was concentrated in vacuum to afford 1-(2-(4-nitrophenoxy)ethyl)piperazine (521 mg, crude, HCl salt) as a white solid. MS(M+H)⁺=252.4

### Step 3. Synthesis of 2-(2,6-dioxopiperidin-3-yl)-4-(4-(2-(4-nitrophenoxy)ethyl)piperazin-1-yl)isoindoline-1,3-dione (6)

To a solution of 2-(2,6-dioxo-3-piperidyl)-4-fluoro-isoindoline-1,3-dione (400 mg, 1.45 mmol) in DMSO (6 mL) were added TEA (439.60 mg, 4.34 mmol, 604.68 μL) and 1-(2-(4-nitrophenoxy)ethyl)piperazine (500.02 mg, crude, HCl salt) at 20° C. and the resulting mixture was stirred at 100° C. for 16 h. LCMS showed starting material was consumed completely and a peak (80%) with desired mass. The reaction mixture was diluted with H₂O (15 mL), yellow solid were precipitated. The solid was collected by filtration. The filter cake was dried in vacuum to afford 2-(2,6-dioxopiperidin-3-yl)-4-(4-(2-(4-nitrophenoxy)ethyl)piperazin-1-yl)isoindoline-1,3-dione (560 mg, 1.08 mmol, 74.68% yield, 98% purity) as a yellow solid. MS(M+H)⁺=508.2

### Step 4. Synthesis of 4-(4-(2-(4-aminophenoxy)ethyl)piperazin-1-yl)-2-(2,6-dioxopiperidin-3-yl)isoindoline-1,3-dione (7)

To a solution of 2-(2,6-dioxopiperidin-3-yl)-4-(4-(2-(4-nitrophenoxy)ethyl)piperazin-1-yl)isoindoline-1,3-dione (400 mg, 788.19 μmol) in CF₃CH₂OH (12 mL) was added Pd/C (100 mg, 10% purity) under N₂ atmosphere. The suspension was degassed and purged with H₂ for 3 times. The mixture was stirred at 20° C. for 16 h under H₂ (15 Psi). LCMS showed starting material was consumed completely and a main peak (94%) with desired mass. The reaction mixture was diluted with CF₃CH₂OH (20 mL) and filtered. The filtrate was concentrated in vacuum to afford 4-(4-(2-(4-aminophenoxy)ethyl)piperazin-1-yl)-2-(2,6-dioxopiperidin-3-yl)isoindoline-1,3-dione (300 mg, 628.26 μmol, 79.71% yield) as a yellow solid. MS(M+H)⁺=478.2

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(2-(2,6-dioxopiperidin-3-yl)-1,3-dioxoisoindolin-4-yl)piperazin-1-yl)ethoxy)phenyl)-2-fluoro-5-methoxybenzamide (Compound 141)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (80 mg, 171.89 μmol) in DMF (2 mL) were added HATU (71.89 mg, 189.07 μmol) and DIPEA (66.65 mg, 515.66 μmol, 89.82 μL). The mixture was stirred at 20° C. for 10 min and a solution of 4-(4-(2-(4-aminophenoxy)ethyl)piperazin-1-yl)-2-(2,6-dioxopiperidin-3-yl)isoindoline-1,3-dione (98.49 mg, crude) in DMF (2 mL) was added and the resulting mixture was stirred at 20° C. for 1 h. LCMS showed starting material was consumed completely and a peak (78%) with desired mass. The reaction mixture was diluted with H₂O (15 mL) and extracted with EtOAc (15 mL×3). The organic layer was washed with brine (15 mL×3), dried over Na₂SO₄, filtered and concentrated. The residue was purified by prep-HPLC (column: Unisil 3-100 C18 Ultra 150*50 mm*3 μm; mobile phase: [water(FA)-ACN]; B %: 20%-50%, 10 min), the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(2-(4-(2-(2,6-dioxopiperidin-3-yl)-1,3-dioxoisoindolin-4-yl)piperazin-1-yl)ethoxy)phenyl)-2-fluoro-5-methoxybenzamide (66.4 mg, 71.79 μmol, 41.77% yield, 95.9% purity) as a yellow solid. MS(M+H)⁺=925.3

¹H NMR (400 MHz, DMSO-d₆) δ=11.08 (br s, 1H), 10.01 (s, 1H), 8.37-8.26 (m, 2H), 8.09 (s, 1H), 7.74-7.67 (m, 1H), 7.63 (d, J=8.9 Hz, 2H), 7.35 (dd, J=5.8, 7.5 Hz, 2H), 7.27 (d, J=6.5 Hz, 1H), 6.95 (d, J=9.0 Hz, 2H), 5.10 (dd, J=5.4, 12.9 Hz, 1H), 4.90-4.79 (m, 1H), 4.18-4.03 (m, 4H), 3.94 (s, 3H), 3.34-3.33 (m, 3H), 3.31-3.30 (m, 4H), 2.93-2.82 (m, 1H), 2.78 (t, J=5.1 Hz, 2H), 2.72-2.68 (m, 4H), 2.65-2.57 (m, 2H), 2.06-1.94 (m, 3H), 1.80-1.70 (m, 2H), 1.69-1.57 (m, 4H).

### Example 142. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-7-fluoro-1,3-dioxoisoindolin-4-yl)piperazin-1-yl)propanoyl)piperidin-4-yl)-3-methoxybenzamide (Compound 142)

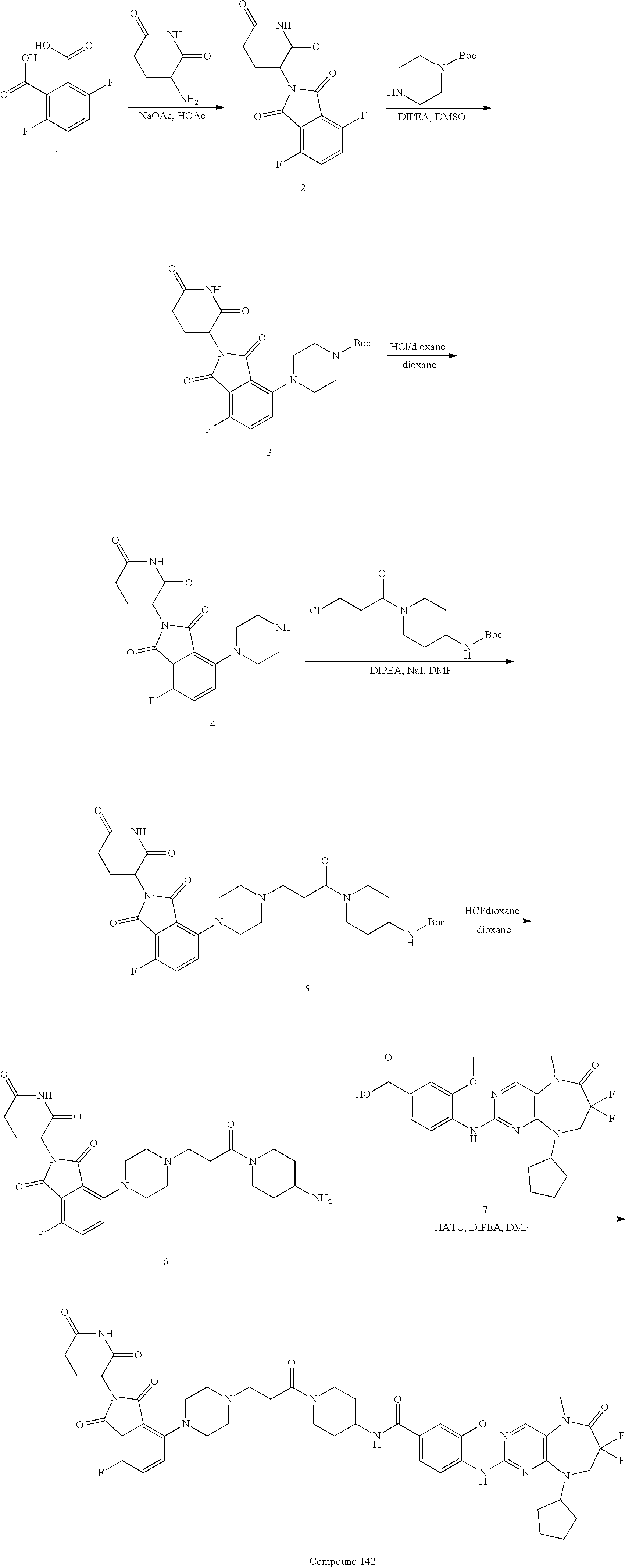

### Step 1. Synthesis of 2-(2,6-dioxopiperidin-3-yl)-4,7-difluoroisoindoline-1,3-dione (2)

A mixture of 3,6-difluorophthalic acid (2 g, 9.90 mmol), 3-aminopiperidine-2,6-dione (2.12 g, 12.86 mmol, HCl) and NaOAc (2.44 g, 29.69 mmol) in AcOH (50 mL) was stirred at 100° C. for 14 h. LCMS showed a main peak with desired mass. The reaction mixture was diluted with water (50 mL) and then filtered. The filter cake was collected and dried to afford 2-(2,6-dioxopiperidin-3-yl)-4,7-difluoroisoindoline-1,3-dione (2.3 g, 7.82 mmol, 79.00% yield) as a brown solid. MS (M+H)⁺=295.1

¹H NMR (400 MHz, DMSO-d₆) δ 11.17 (s, 1H), 7.83 (t, J=5.8 Hz, 2H), 5.16 (dd, J=5.4, 12.9 Hz, 1H), 2.96-2.82 (m, 1H), 2.65-2.57 (m, 1H), 2.50-2.43 (m, 1H), 2.13-2.00 (m, 1H).

### Step 2. Synthesis of tert-butyl 4-(2-(2,6-dioxopiperidin-3-yl)-7-fluoro-1,3-dioxoisoindolin-4-yl) piperazine-1-carboxylate (3)

To the solution of 2-(2,6-dioxopiperidin-3-yl)-4,7-difluoroisoindoline-1,3-dione (1 g, 3.40 mmol) and tert-butyl piperazine-1-carboxylate (696.36 mg, 3.74 mmol) in DMSO (10 mL) was added DIPEA (878.58 mg, 6.80 mmol, 1.18 mL) and the resulting mixture was stirred at 100° C. for 12 h. LCMS showed a main peak with desired mass. The mixture was poured into water (50 mL) and extracted with EtOAc (30 mL×3). The combined organic layer was washed with brine (50 mL), dried over Na₂SO₄, filtered and concentrated. The residue was triturated with MBTE (20 mL) to afford tert-butyl 4-(2-(2,6-dioxopiperidin-3-yl)-7-fluoro-1,3-dioxoisoindolin-4-yl) piperazine-1-carboxylate (1.5 g, 3.03 mmol, 89.13% yield, 93% purity) as a yellow solid. MS (M+H)⁺=461.2

### Step 3. Synthesis of 2-(2,6-dioxopiperidin-3-yl)-4-fluoro-7-(piperazin-1-yl)isoindoline-1,3-dione (4)

To the solution of tert-butyl 4-(2-(2,6-dioxopiperidin-3-yl)-7-fluoro-1,3-dioxoisoindolin-4-yl) piperazine-1-carboxylate (1.5 g, 3.26 mmol) in dioxane (10 mL) was added HCl/dioxane (4 M, 10 mL) and the resulting mixture was stirred at 20° C. for 1 h. LCMS showed a main peak with desired mass. The reaction was concentrated under reduced pressure to afford 2-(2,6-dioxopiperidin-3-yl)-4-fluoro-7-(piperazin-1-yl)isoindoline-1,3-dione (1.3 g, crude, HCl) as a yellow solid. MS(M+H)⁺=361.2.

### Step 4. Synthesis of tert-butyl (1-(3-(4-(2-(2,6-dioxopiperidin-3-yl)-7-fluoro-1,3-dioxoisoindolin-4-yl)piperazin-1-yl)propanoyl)piperidin-4-yl)carbamate (5)

To a solution of 2-(2,6-dioxopiperidin-3-yl)-4-fluoro-7-(piperazin-1-yl)isoindoline-1,3-dione (0.5 g, 1.26 mmol, HCl) in DMF (10 mL) were added tert-butyl (1-(3-chloropropanoyl)piperidin-4-yl)carbamate (1.10 g, 3.78 mmol), DIPEA (488.57 mg, 3.78 mmol, 658.45 μL) and NaI (18.89 mg, 126.01 μmol), the mixture was stirred at 70° C. for 16 h. LCMS showed a peak (40%) with desired mass. The mixture was poured into water (50 mL) and extracted with EtOAc (30 mL×3), the combined organic layer was washed with brine (50 mL), dried over Na₂SO₄, filtered and concentrated. The residue was purified by flash silica gel chromatography (25 g SepaFlash® Silica Flash Column, Eluent of 0˜50% MeOH/EtOAc gradient @80 mL/min) to afford tert-butyl (1-(3-(4-(2-(2,6-dioxopiperidin-3-yl)-7-fluoro-1,3-dioxoisoindolin-4-yl)piperazin-1-yl) propanoyl)piperidin-4-yl)carbamate (260 mg, 372.24 μmol, 29.54% yield, 88% purity) as a yellow solid. MS(M+H)⁺=615.4

### Step 5. Synthesis of 4-(4-(3-(4-aminopiperidin-1-yl)-3-oxopropyl)piperazin-1-yl)-2-(2,6-dioxopiperidin-3-yl)-7-fluoroisoindoline-1,3-dione (6)

To a solution of tert-butyl (1-(3-(4-(2-(2,6-dioxopiperidin-3-yl)-7-fluoro-1,3-dioxoisoindolin-4-yl)piperazin-1-yl) propanoyl)piperidin-4-yl)carbamate (260 mg, 423.00 μmol) in dioxane (5 mL) was added HCl/dioxane (4 M, 5 mL) and the resulting mixture was stirred at 20° C. for 12 h. LCMS showed a main peak with desired mass. The reaction mixture was concentrated under reduced pressure to afford 4-(4-(3-(4-aminopiperidin-1-yl)-3-oxopropyl)piperazin-1-yl)-2-(2,6-dioxopiperidin-3-yl)-7-fluoroisoindoline-1,3-dione (230 mg, 417.42 μmol, 98.68% yield, HCl) as a yellow solid. MS (M+H)⁺=515.2

### Step 6. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-7-fluoro-1,3-dioxoisoindolin-4-yl)piperazin-1-yl)propanoyl)piperidin-4-yl)-3-methoxybenzamide (Compound 142)

To a solution of 4-(4-(3-(4-aminopiperidin-1-yl)-3-oxopropyl)piperazin-1-yl)-2-(2,6-dioxopiperidin-3-yl)-7-fluoroisoindoline-1,3-dione (110 mg, 199.63 μmol, HCl) and 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (89.32 mg, 199.63 μmol) in DMF (2.5 mL) was added HATU (83.50 mg, 219.60 μmol) and DIPEA (77.40 mg, 598.90 μmol, 104.32 μL) and the resulting mixture was stirred at 20° C. for 1 h. LCMS showed that starting material was consumed and the desired mass. The mixture was poured into water (10 mL) and extracted with EtOAc (6 mL×3), the combined organic layer was washed with brine (10 mL), dried over Na₂SO₄, filtered and concentrated. The residue was purified by prep-HPLC (column: Waters Xbridge C18 150*50 mm*10 μm; mobile phase: [water(NH₄HCO₃)-ACN]; B %: 31%-61%, 10 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-7-fluoro-1,3-dioxoisoindolin-4-yl)piperazin-1-yl)propanoyl)piperidin-4-yl)-3-methoxybenzamide (23.8 mg, 23.90 μmol, 11.97% yield, 94.8% purity) as a yellow solid. MS(M+H)⁺=944.1

¹H NMR (400 MHz, DMSO-d₆) δ=11.11 (s, 1H), 8.31-8.25 (m, 2H), 8.19-8.13 (m, 1H), 7.98 (s, 1H), 7.62-7.57 (m, 1H), 7.51-7.47 (m, 2H), 7.41 (dd, J=3.8, 9.4 Hz, 1H), 5.10 (dd, J=5.5, 12.8 Hz, 1H), 4.85-4.71 (m, 1H), 4.45-4.36 (m, 1H), 4.11-3.95 (m, 4H), 3.94 (s, 3H), 3.32 (br s, 3H), 3.24-3.13 (m, 4H), 2.89-2.86 (m, 1H), 2.66-2.54 (m, 12H), 2.08-1.77 (m, 5H), 1.75-1.67 (m, 2H), 1.61-1.58 (m, 4H), 1.51-1.34 (m, 2H).

### Example 143. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(4-(2-(2,6-dioxopiperidin-3-yl)-1,3-dioxoisoindolin-4-yl)piperazin-1-yl)ethyl)piperidin-3-yl)-3-methoxybenzamide (Compound 143)

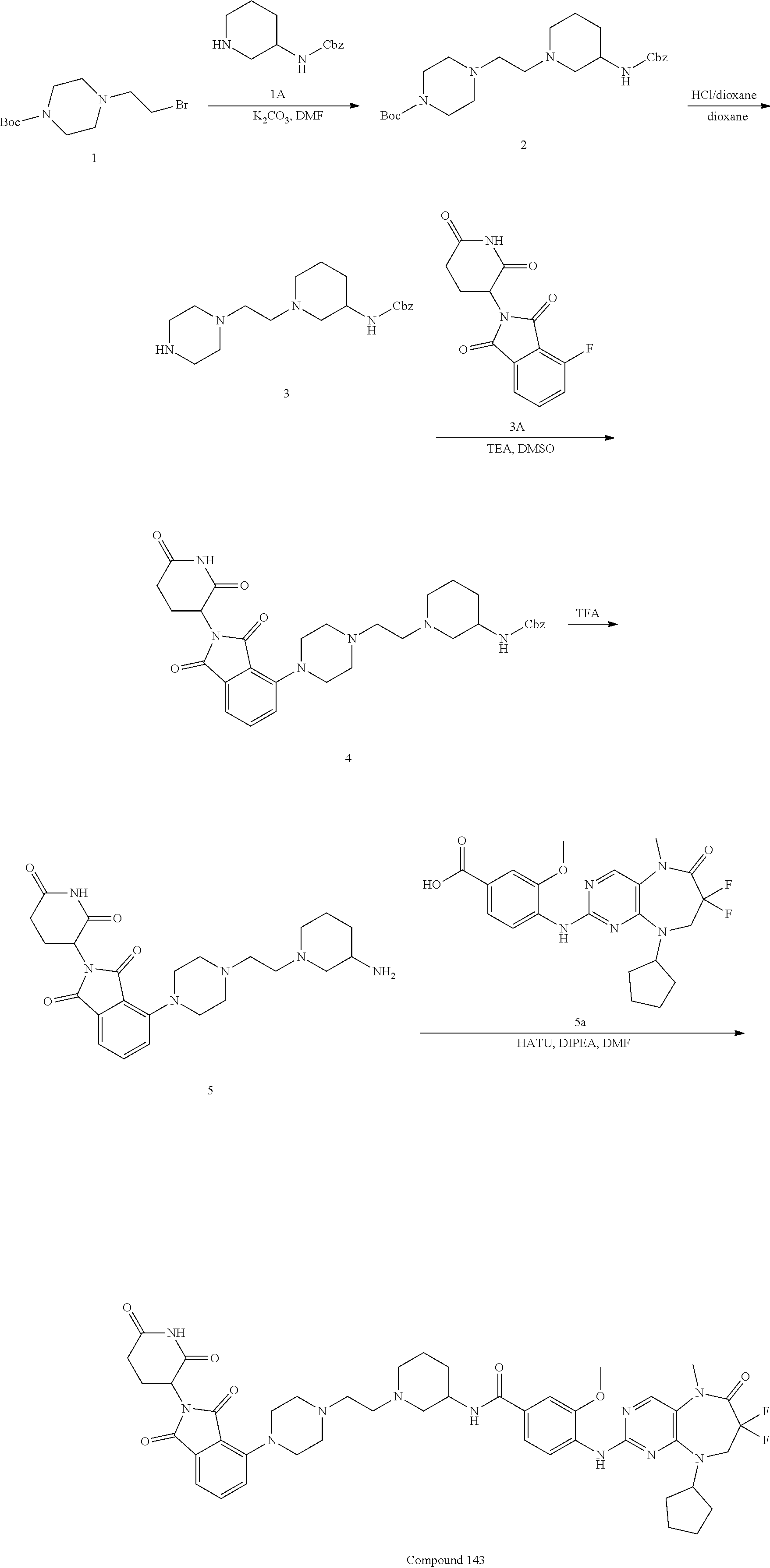

The compound 143 was synthesized by the method described in the scheme similar to the method described in Example 53.

MS(M+H)⁺=898.3, ¹H NMR (400 MHz, DMSO-d₆) δ=11.09 (s, 1H), 8.32-8.21 (m, 2H), 8.04 (br d, J=7.9 Hz, 1H), 7.97 (s, 1H), 7.75-7.63 (m, 1H), 7.57-7.44 (m, 2H), 7.33 (dd, J=7.8, 11.6 Hz, 2H), 5.09 (dd, J=5.4, 12.8 Hz, 1H), 4.76 (quin, J=8.2 Hz, 1H), 4.05 (br t, J=14.0 Hz, 2H), 3.94 (s, 4H), 3.33 (br s, 3H), 3.28 (br s, 4H), 2.96-2.75 (m, 3H), 2.66-2.56 (m, 5H), 2.52-2.48 (m, 5H), 2.09-1.86 (m, 5H), 1.82-1.75 (m, 1H), 1.77-1.66 (m, 3H), 1.65-1.45 (m, 5H), 1.44-1.31 (m, 1H).

### Example 144. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(4-(2-(2,6-dioxopiperidin-3-yl)-1,3-dioxoisoindolin-4-yl)piperazin-1-yl)propyl)piperidin-3-yl)-3-methoxybenzamide (Compound 144)

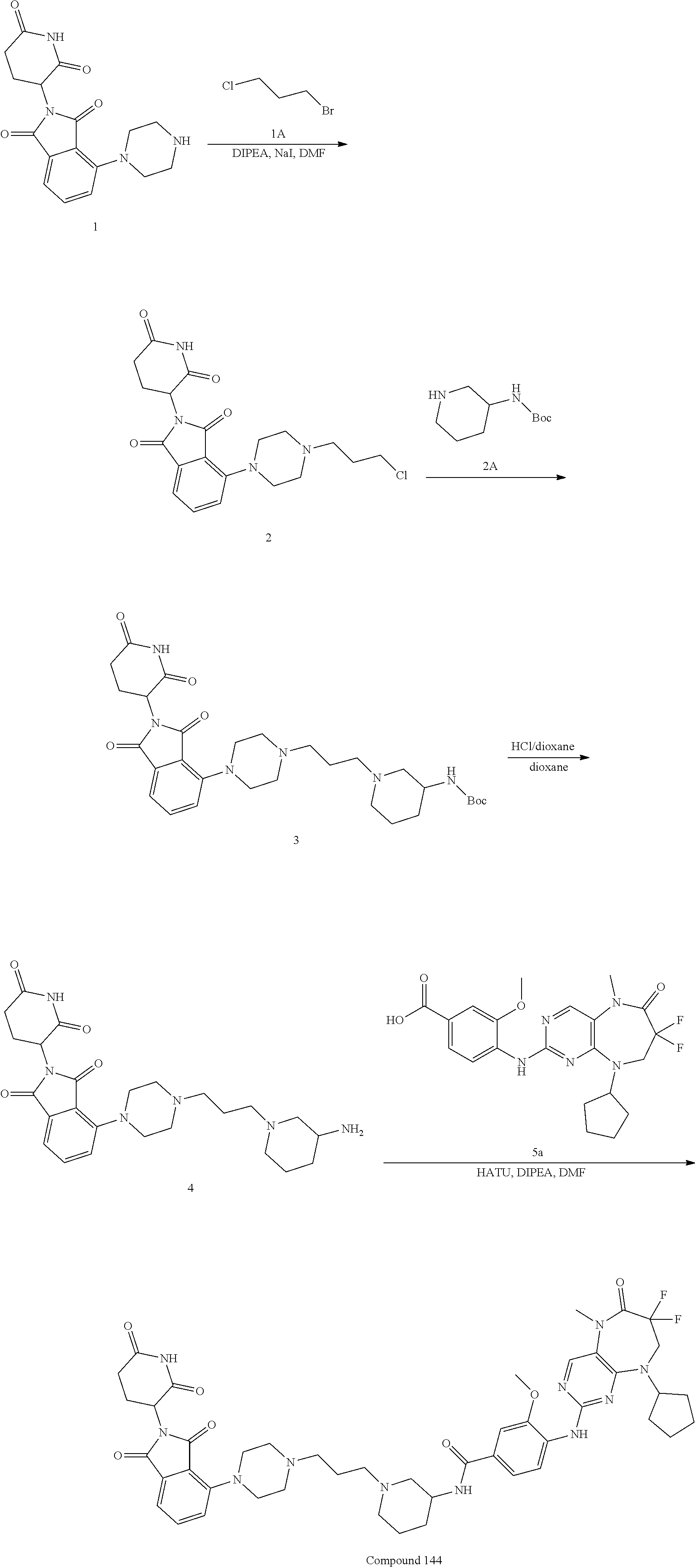

The compound 144 was synthesized by the method described in the scheme similar to the method described in Example 60.

MS(M+H)⁺=912.3, ¹H NMR (400 MHz, DMSO-d₆) δ=11.09 (s, 1H), 8.35-8.18 (m, 2H), 8.04 (br d, J=7.9 Hz, 1H), 7.96 (s, 1H), 7.69 (dd, J=7.3, 8.3 Hz, 1H), 7.56-7.42 (m, 2H), 7.40-7.23 (m, 2H), 5.09 (dd, J=5.4, 12.8 Hz, 1H), 4.84-4.67 (m, 1H), 4.04 (br t, J=14.0 Hz, 2H), 3.98-3.85 (m, 4H), 3.32 (br s, 3H), 3.31-3.27 (m, 4H), 2.96-2.82 (m, 2H), 2.80-2.76 (m, 1H), 2.62-2.52 (m, 6H), 2.39-2.30 (m, 4H), 2.07-1.99 (m, 1H), 1.98-1.90 (m, 2H), 1.90-1.77 (m, 3H), 1.65-1.60 (m, 3H), 1.66-1.46 (m, 7H), 1.35-1.25 (m, 1H).

### Example 145. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-(2-(2,6-dioxopiperidin-3-yl)-1,3-dioxoisoindolin-4-yl)piperazine-1-carbonyl)piperidin-3-yl)-3-methoxybenzamide (Compound 145)

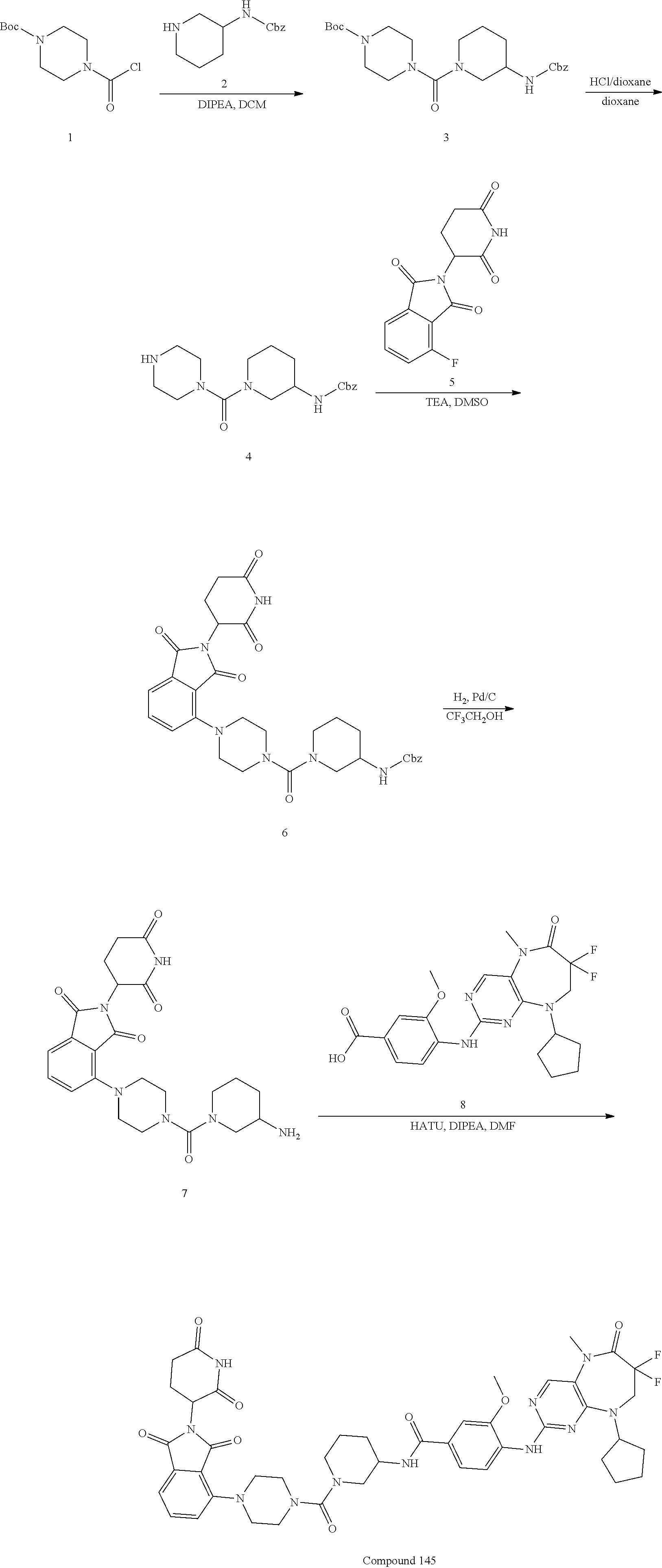

### Step 1. Synthesis of tert-butyl 4-(3-(((benzyloxy)carbonyl)amino)piperidine-1-carbonyl) piperazine-1-carboxylate (3)

To a solution of benzyl piperidin-3-ylcarbamate (100 mg, 426.82 μmol) in DCM (2 mL) were added tert-butyl 4-(chlorocarbonyl)piperazine-1-carboxylate (100 mg, 402.08 μmol) and DIPEA (155.90 mg, 1.21 mmol, 210.11 μL), the resulting mixture was stirred at 20° C. for 4 h. LCMS showed a main peak with desired mass. The reaction mixture was diluted with H₂O (2 mL), the organic phase was separated, the aqueous was extracted with EtOAc (2 mL×3). The combined organic layers were washed with brine (2 mL×2), dried over Na₂SO₄, filtered. The filtrate was concentrated under reduced pressure to give a residue. The residue was purified by flash silica gel chromatography (Biotage; 10 g SepaFlash® Silica Flash Column, Eluent of 20˜60% EtOAc/Petroleum ether @40 mL/min) to afford tert-butyl 4-(3-(((benzyloxy)carbonyl)amino)piperidine-1-carbonyl)piperazine-1-carboxylate (160 mg, 318.90 μmol, 79.31% yield, 89% purity) as a white solid. MS(M+H)⁺=447.0

### Step 2. Synthesis of benzyl (1-(piperazine-1-carbonyl)piperidin-3-yl)carbamate (4)

To a solution of tert-butyl 4-(3-(((benzyloxy)carbonyl)amino)piperidine-1-carbonyl)piperazine-1-carboxylate (160 mg, 358.31 μmol) in dioxane (2 mL) was added HCl/dioxane (4 M, 2 mL), the mixture was stirred at 20° C. for 1 h. LCMS showed a main peak with desired mass. The mixture was concentrated under reduced pressure to afford benzyl (1-(piperazine-1-carbonyl)piperidin-3-yl)carbamate (120 mg, crude) as a white solid. MS(M+H)⁺=347.0

### Step 3. Synthesis of benzyl (1-(4-(2-(2,6-dioxopiperidin-3-yl)-1,3-dioxoisoindolin-4-yl)piperazine-1-carbonyl)piperidin-3-yl)carbamate (6)

To a solution of benzyl (1-(piperazine-1-carbonyl)piperidin-3-yl)carbamate (120 mg, 346.40 μmol) in DMSO (2 mL) were added 2-(2,6-dioxopiperidin-3-yl)-4-fluoroisoindoline-1,3-dione (95.68 mg, 346.40 μmol), TEA (105.15 mg, 1.04 mmol, 144.64 μL), the mixture was stirred at 100° C. for 16 h. LCMS showed a peak (41%) with desired mass. The reaction mixture was diluted with brine (10 mL), extracted with EtOAc (10 mL×3). The combined organic layers were washed with brine (20 mL×2), dried over Na₂SO₄, filtered, and the filtrate was concentrated under reduced pressure to give a residue. The residue was purified by flash silica gel chromatography (Biotage; 10 g SepaFlash® Silica Flash Column, Eluent of 10˜60% EtOAc/Petroleum ether @40 mL/min) to afford benzyl (1-(4-(2-(2,6-dioxopiperidin-3-yl)-1,3-dioxoisoindolin-4-yl)piperazine-1-carbonyl)piperidin-3-yl)carbamate (150 mg, 243.93 μmol, 70.42% yield, 98% purity) as a yellow solid. MS(M+H)⁺=603.0

### Step 4. Synthesis of 4-(4-(3-aminopiperidine-1-carbonyl)piperazin-1-yl)-2-(2,6-dioxopiperidin-3-yl)isoindoline-1,3-dione (7)

A mixture of benzyl (1-(4-(2-(2,6-dioxopiperidin-3-yl)-1,3-dioxoisoindolin-4-yl)piperazine-1-carbonyl)piperidin-3-yl)carbamate (100 mg, 165.94 μmol) and Pd/C (20 mg, 10% purity) in CF₃CH₂OH (10 mL) was degassed and purged with H₂ for 3 times, the resulting mixture was stirred at 20° C. for 16 h under H₂ (15 Psi) atmosphere. LCMS showed a main peak with desired mass. The mixture was filtered through a pad of celite and the filtrate was concentrated in vacuum to afford 4-(4-(3-aminopiperidine-1-carbonyl)piperazin-1-yl)-2-(2,6-dioxopiperidin-3-yl) isoindoline-1,3-dione (50 mg, crude) as a yellow solid. MS(M+H)⁺=469.0

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-(2-(2,6-dioxopiperidin-3-yl)-1,3-dioxoisoindolin-4-yl)piperazine-1-carbonyl)piperidin-3-yl)-3-methoxybenzamide (Compound 145)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (47.75 mg, 106.72 μmol) in DMF (1 mL) were added HATU (60.87 mg, 160.08 μmol) and DIPEA (41.38 mg, 320.17 μmol, 55.77 μL), the mixture was stirred at 20° C. for 0.5 h. 4-(4-(3-aminopiperidine-1-carbonyl)piperazin-1-yl)-2-(2,6-dioxopiperidin-3-yl)isoindoline-1,3-dione (50 mg, 106.72 μmol) was added, the resulting mixture was stirred at 20° C. for 16 h. LCMS showed a main peak with desired mass. The reaction mixture was diluted with brine (3 mL), extracted with EtOAc (3 mL×3). The combined organic layers were washed with brine (5 mL×2), dried over Na₂SO₄, filtered. The filtrate was concentrated under reduced pressure to give a residue. The residue was purified by prep-TLC (SiO₂, DCM:MeOH=10:1) and re-purified by reversed-phase HPLC (column: Waters Xbridge 150*25 mm*5 μm; mobile phase: [water(NH₄HCO₃)-ACN]; B %: 41%-71%, 8 min). The eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-(2-(2,6-dioxopiperidin-3-yl)-1,3-dioxoisoindolin-4-yl)piperazine-1-carbonyl)piperidin-3-yl)-3-methoxybenzamide (30.2 mg, 31.28 μmol, 29.31% yield, 93% purity) as a yellow solid. MS(M+H)⁺=898.0

¹H NMR (400 MHz, DMSO-d₆) δ=11.09 (s, 1H), 8.34-8.21 (m, 3H), 7.98 (s, 1H), 7.76-7.69 (m, 1H), 7.53-7.46 (m, 2H), 7.42-7.33 (m, 2H), 5.16-5.07 (m, 1H), 4.83-4.72 (m, 1H), 4.05 (t, J=14.1 Hz, 2H), 3.97-3.84 (m, 4H), 3.70-3.63 (m, 1H), 3.58-3.49 (m, 1H), 3.43-3.38 (m, 4H), 3.33-3.25 (m, 7H), 2.95-2.77 (m, 3H), 2.64-2.56 (m, 2H), 2.09-1.88 (m, 4H), 1.81-1.51 (m, 9H).

### Example 146. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((3R)-1-(2-(4-(2-(2,6-dioxopiperidin-3-yl)-1,3-dioxoisoindolin-4-yl)piperazin-1-yl)acetyl)piperidin-3-yl)-3-methoxybenzamide (Compound 146)

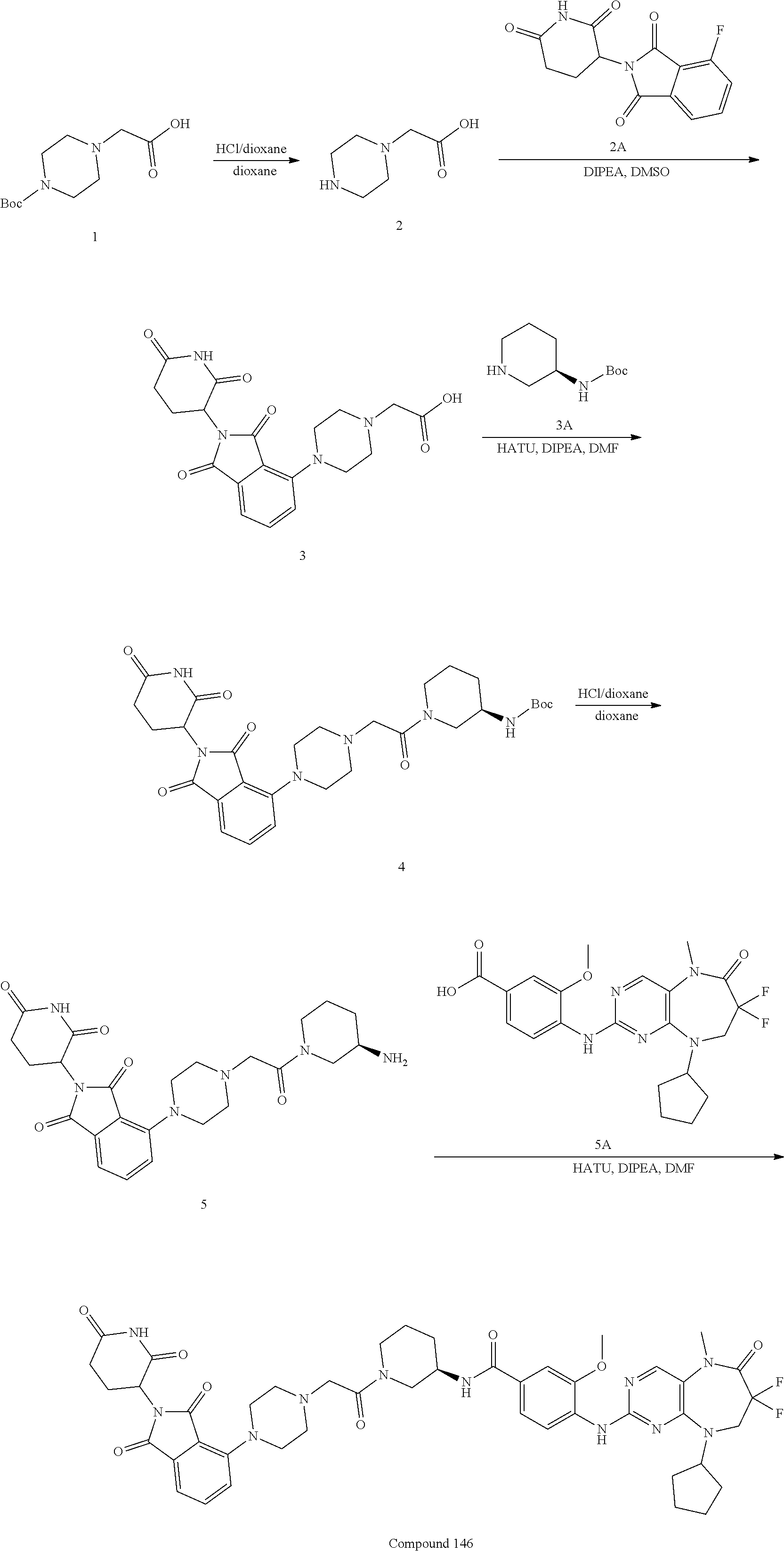

### Step 1. Synthesis of 2-(piperazin-1-yl)acetic acid (2)

To a solution of 2-(4-(tert-butoxycarbonyl)piperazin-1-yl)acetic acid (1 g, 4.09 mmol) in dioxane (5 mL) was added HCl/dioxane (4 M, 20 mL). The mixture was stirred at 25° C. for 2 hrs. TLC (Petroleum ether:EtOAc=1:1) indicated one new spot was formed. The reaction mixture was concentrated under reduced pressure to afford 2-(piperazin-1-yl)acetic acid (600 mg, 3.95 mmol, 96.58% yield, 95% purity, HCl salt) as a white solid.

### Step 2. Synthesis of 2-(4-(2-(2,6-dioxopiperidin-3-yl)-1,3-dioxoisoindolin-4-yl)piperazin-1-yl)acetic acid (3)

To a solution of 2-(piperazin-1-yl)acetic acid (500 mg, 3.47 mmol) in DMSO (5 mL) was added DIPEA (1.34 g, 10.40 mmol, 1.81 mL) and 2-(2,6-dioxopiperidin-3-yl)-4-fluoroisoindoline-1,3-dione (957.96 mg, 3.47 mmol). The mixture was stirred at 80° C. for 16 hrs. LCMS showed a peak with desired mass. The reaction mixture was diluted with water (20 mL) and extracted with EtOAc (20 mL×3). The combined organic layers were washed with bine (10 mL×3), dried over Na₂SO₄, filtered. The filtrate was concentrated under reduced pressure. The residue was purified by prep-HPLC (column: Phenomenex luna C18 150*40 mm*15 μm; mobile phase: [water(TFA)-ACN]; B %: 1%-25%, 10 min), the eluent was freeze dried to afford 2-(4-(2-(2,6-dioxopiperidin-3-yl)-1,3-dioxoisoindolin-4-yl)piperazin-1-yl)acetic acid (1 g, 2.50 mmol, 72.02% yield) as a yellow solid. MS(M+H)⁺=400.9

### Step 3. Synthesis of tert-butyl ((3R)-1-(2-(4-(2-(2,6-dioxopiperidin-3-yl)-1,3-dioxoisoindolin-4-yl)piperazin-1-yl)acetyl)piperidin-3-yl)carbamate (4)

To a solution of 2-(4-(2-(2,6-dioxopiperidin-3-yl)-1,3-dioxoisoindolin-4-yl)piperazin-1-yl)acetic acid (350 mg, 874.16 μmol) in DMF (3 mL) were added HATU (398.86 mg, 1.05 mmol) and DIPEA (225.96 mg, 1.75 mmol, 304.53 μL). The mixture was stirred at 25° C. for 10 min, then a solution of tert-butyl (R)-piperidin-3-ylcarbamate (175.07 mg, 874.16 μmol) and DIPEA (112.98 mg, 874.16 μmol, 152.26 μL) in DMF (2 mL) was added dropwise at 25° C. The resulting mixture was stirred at 25° C. for 1 hr. LCMS showed a peak with desired mass. The reaction mixture filtered. The filtrate was concentrated. The residue was purified by prep-HPLC (column: Phenomenex luna C18 150*40 mm*15 μm; mobile phase: [water(TFA)-ACN]; B %: 12%-42%, 10 min), the eluent was freeze dried to afford tert-butyl ((3R)-1-(2-(4-(2-(2,6-dioxopiperidin-3-yl)-1,3-dioxoisoindolin-4-yl)piperazin-1-yl)acetyl)piperidin-3-yl)carbamate (400 mg, 686.52 μmol, 78.54% yield) as a yellow solid. MS(M+H)⁺=583.1

### Step 4. Synthesis of 4-(4-(2-((R)-3-aminopiperidin-1-yl)-2-oxoethyl)piperazin-1-yl)-2-(2,6-dioxopiperidin-3-yl)isoindoline-1,3-dione (5)

To a solution of tert-butyl ((3R)-1-(2-(4-(2-(2,6-dioxopiperidin-3-yl)-1,3-dioxoisoindolin-4-yl)piperazin-1-yl)acetyl)piperidin-3-yl)carbamate (80 mg, 137.30 μmol) in dioxane (1 mL) was added HCl/dioxane (4 M, 34.33 μL). The mixture was stirred at 25° C. for 2 hrs. LCMS showed a peak with desired mass. The reaction mixture was concentrated under reduced pressure to afford 4-(4-(2-((R)-3-aminopiperidin-1-yl)-2-oxoethyl)piperazin-1-yl)-2-(2,6-dioxopiperidin-3-yl)isoindoline-1,3-dione (66 mg, 136.78 μmol, 99.62% yield, HCl salt) as a yellow solid. MS(M+H)⁺=483.3

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((3R)-1-(2-(4-(2-(2,6-dioxopiperidin-3-yl)-1,3-dioxoisoindolin-4-yl)piperazin-1-yl)acetyl)piperidin-3-yl)-3-methoxybenzamide (Compound 146)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (61.20 mg, 136.78 μmol) in DMF (1 mL) were added HATU (62.41 mg, 164.13 μmol) and DIPEA (88.39 mg, 683.89 μmol, 119.12 μL). The mixture was stirred at 25° C. for 10 min, then a solution of 4-(4-(2-((R)-3-aminopiperidin-1-yl)-2-oxoethyl)piperazin-1-yl)-2-(2,6-dioxopiperidin-3-yl)isoindoline-1,3-dione (66 mg, 136.78 μmol, HCl salt) in DMF (0.5 mL) was added. The mixture was stirred at 25° C. for 2 h. LCMS showed a peak with desired mass. The mixture was filtered. The filtrate was purified by prep-HPLC (column: Waters Xbridge 150*25 mm*5 μm; mobile phase: [water(NH₄HCO₃)-ACN]; B %: 37%-67%, 10 min), the eluent was freeze dried to afford 4-((9-cyclopentyl-7,7-difluoro-5-methyl-6-oxo-6,7,8,9-tetrahydro-5H-pyrimido[4,5-b][1,4]diazepin-2-yl)amino)-N-((3R)-1-(2-(4-(2-(2,6-dioxopiperidin-3-yl)-1,3-dioxoisoindolin-4-yl)piperazin-1-yl)acetyl)piperidin-3-yl)-3-methoxybenzamide (29 mg, 30.21 μmol, 22.09% yield, 95% purity) as a yellow solid. MS(M+H)⁺=912.6

¹H NMR (400 MHz, DMSO-d₆) δ=10.82 (s, 1H), 8.29-8.25 (m, 1H), 8.25-8.24 (m, 1H), 7.93-7.88 (m, 1H), 7.86 (s, 1H), 7.70-7.64 (m, 1H), 7.51-7.46 (m, 2H), 7.35-7.28 (m, 2H), 5.09-5.01 (m, 1H), 4.80-4.70 (m, 1H), 4.08-3.96 (m, 4H), 3.95 (s, 3H), 3.46-3.14 (m, 12H), 2.92-2.85 (m, 1H), 2.70-2.61 (m, 6H), 2.10-2.03 (m, 1H), 2.00-2.93 (m, 3H), 1.84-1.77 (m, 1H), 1.74-1.69 (m, 2H), 1.67-1.56 (m, 6H).

### Example 147. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((3S)-1-(2-(4-(2-(2,6-dioxopiperidin-3-yl)-1,3-dioxoisoindolin-4-yl)piperazin-1-yl)acetyl)piperidin-3-yl)-3-methoxybenzamide (Compound 147)

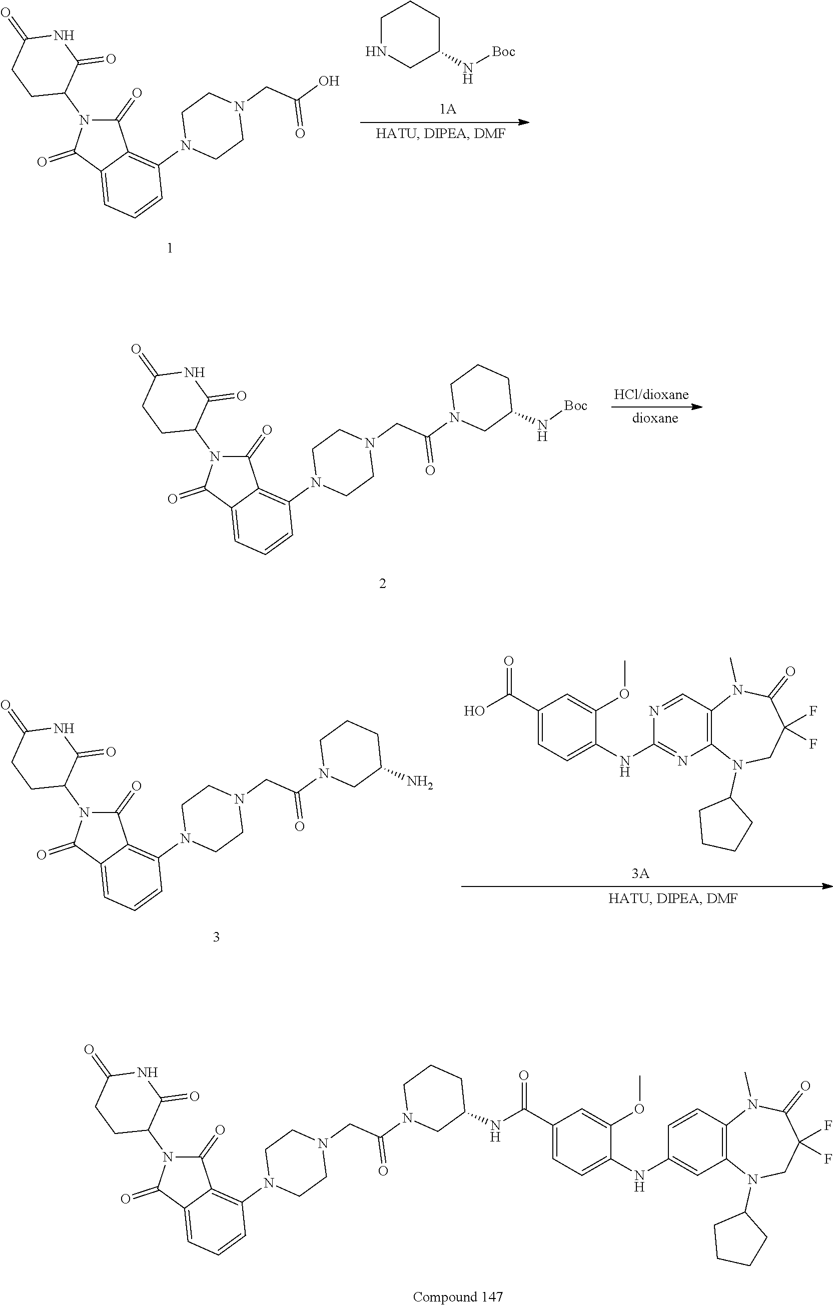 [SMILES: O=C(O)CN1CCN(c2cccc3c2C(=O)N(C2CCC(=O)NC2=O)C3=O)CC1 ; CC(C)(C)OC(=O)N[C@H]1CCCNC1 ; CC(C)(C)OC(=O)N[C@H]1CCCN(C(=O)CN2CCN(c3cccc4c3C(=O)N(C3CCC(=O)NC3=O)C4=O)CC2)C1 ; N[C@H]1CCCN(C(=O)CN2CCN(c3cccc4c3C(=O)N(C3CCC(=O)NC3=O)C4=O)CC2)C1 ; COc1cc(C(=O)O)ccc1Nc1ncc2c(n1)N(C1CCCC1)CC(F)(F)C(=O)N2C ; COc1cc(C(=O)N[C@H]2CCCN(C(=O)CN3CCN(c4cccc5c4C(=O)N(C4CCC(=O)NC4=O)C5=O)CC3)C2)ccc1Nc1ccc2c(c1)N(C1CCCC1)CC(F)(F)C(=O)N2C]

The compound 147 was synthesized by the method described in the scheme similar to the method described in Example 146.

MS(M+H)⁺=912.4, ¹H NMR (400 MHz, DMSO-d₆) δ=10.98-10.69 (m, 1H), 8.44-8.15 (m, 2H), 8.02-7.83 (m, 2H), 7.72-7.62 (m, 1H), 7.56-7.45 (m, 2H), 7.41-7.20 (m, 2H), 5.16-4.98 (m, 1H), 4.86-4.69 (m, 1H), 4.22-3.98 (m, 4H), 3.98-3.92 (m, 3H), 3.53-3.20 (m, 12H), 2.92-2.85 (m, 1H), 2.72-2.63 (m, 6H), 2.10-1.92 (m, 4H), 1.87-1.54 (m, 9H).

### Example 148. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-((4-(2-(2,6-dioxopiperidin-3-yl)-1,3-dioxoisoindolin-5-yl)piperazin-1-yl)methyl)piperidin-1-yl)-3-methoxybenzamide (Compound 148)

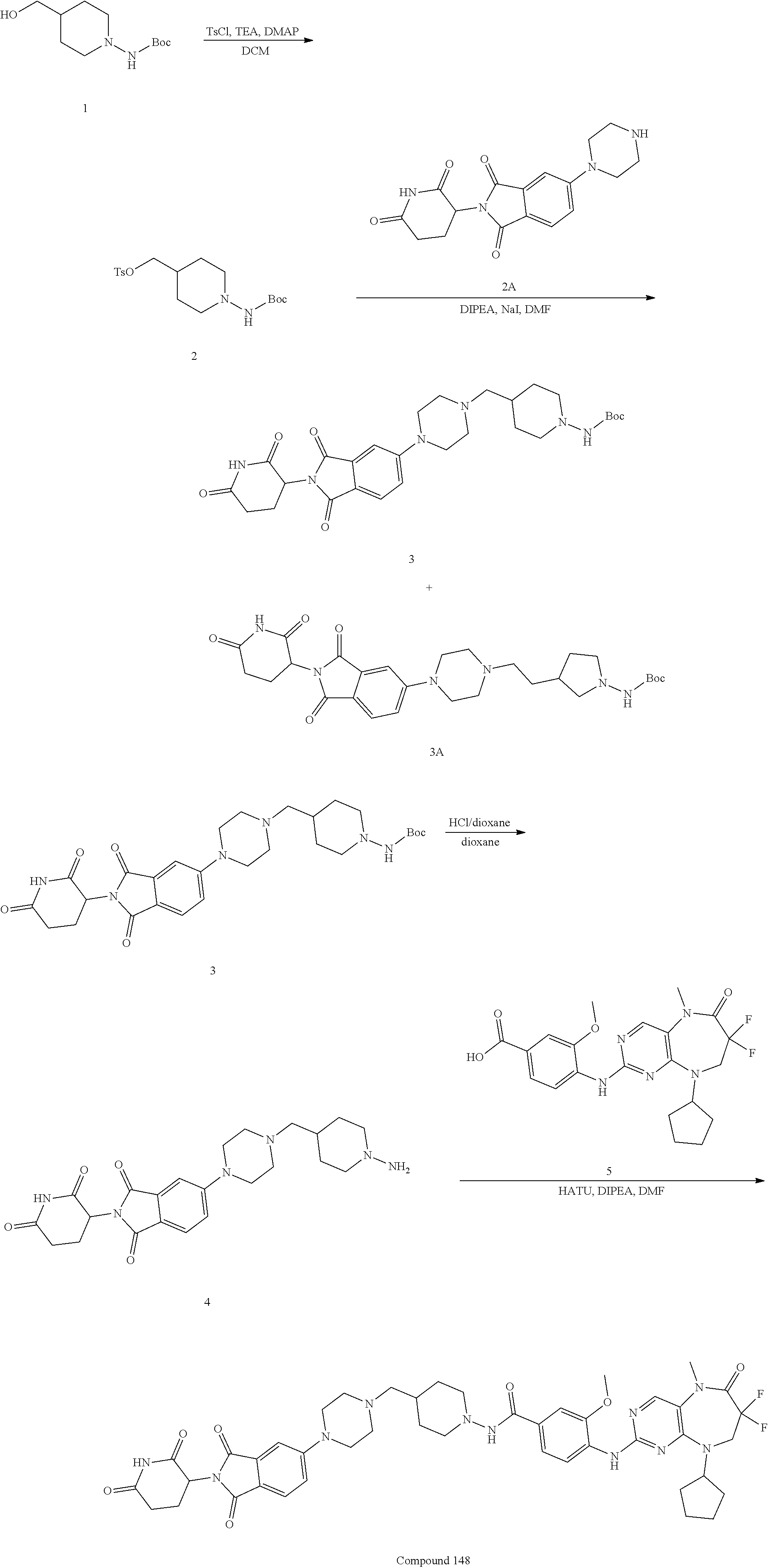

### Step 1. Synthesis of (1-((tert-butoxycarbonyl)amino)piperidin-4-yl)methyl 4-methylbenzenesulfonate (2)

To a solution of tert-butyl (4-(hydroxymethyl)piperidin-1-yl)carbamate (2.5 g, 10.86 mmol) in DCM (20 mL) were added 4-methylbenzenesulfonyl chloride (3.10 g, 16.28 mmol), TEA (3.30 g, 32.57 mmol, 4.53 mL) and DMAP (132.62 mg, 1.09 mmol), the mixture was stirred at 25° C. for 16 hr. LCMS showed desired mass, the mixture was diluted with water (3 mL), and extracted with EtOAc (5 mL×3). The organic layer was dried over Na₂SO₄, filtered and concentrated in vacuum. The crude product was purified by flash silica gel chromatography (Biotage, 4 g SepaFlash® Silica Flash Column, Eluent of 4˜80% EtOAc/Petroleum ether gradient @20 mL/min) to afford (1-((tert-butoxycarbonyl)amino)piperidin-4-yl)methyl 4-methylbenzenesulfonate (1.26 g, 2.95 mmol, 27.17% yield, 90% purity) as a white solid. MS(M+H)⁺=385.2

### Step 2. Synthesis of tert-butyl (4-((4-(2-(2,6-dioxopiperidin-3-yl)-1,3-dioxoisoindolin-5-yl)piperazin-1-yl)methyl) piperidin-1-yl)carbamate (3) and tert-butyl (3-(2-(4-(2-(2,6-dioxopiperidin-3-yl)-1,3-dioxoisoindolin-5-yl)piperazin-1-yl)ethyl) pyrrolidin-1-yl)carbamate (3A)

To a solution of (1-((tert-butoxycarbonyl)amino)piperidin-4-yl)methyl 4-methylbenzenesulfonate (500 mg, 1.30 mmol) and 2-(2,6-dioxopiperidin-3-yl)-5-(piperazin-1-yl)isoindoline-1,3-dione (534.24 mg, 1.56 mmol) in DMF (20 mL) were added DIPEA (504.21 mg, 3.90 mmol, 679.53 μL) and NaI (38.99 mg, 260.09 μmol), the mixture was stirred at 80° C. for 16 hr. LCMS showed the starting material consumed completely, and a peak with desired mass. The reaction mixture was diluted with water (10 mL), extracted with EtOAc (15 mL×3). The combined organic layers were dried over Na₂SO₄, filtered and concentrated in vacuum. The crude product was purified by flash silica gel chromatography (Biotage, 4 g SepaFlash® Silica Flash Column, Eluent of 4˜10% MeOH/DCM gradient @20 mL/min) to afford tert-butyl (4-((4-(2-(2,6-dioxopiperidin-3-yl)-1,3-dioxoisoindolin-5-yl)piperazin-1-yl)methyl)piperidin-1-yl)carbamate (120 mg, 212.03 μmol, 16.30% yield, 98% purity) as a yellow solid and tert-butyl (3-(2-(4-(2-(2,6-dioxopiperidin-3-yl)-1,3-dioxoisoindolin-5-yl)piperazin-1-yl)ethyl)pyrrolidin-1-yl)carbamate (240 mg, 389.44 μmol, 29.95% yield, 90% purity) as a yellow solid. MS(M+H)⁺=555.1

### Step 3. Synthesis of 5-(4-((1-aminopiperidin-4-yl)methyl)piperazin-1-yl)-2-(2,6-dioxopiperidin-3-yl)isoindoline-1,3-dione (4)

To a solution of tert-butyl (4-((4-(2-(2,6-dioxopiperidin-3-yl)-1,3-dioxoisoindolin-5-yl)piperazin-1-yl)methyl)piperidin-1-yl)carbamate (120 mg, 216.36 μmol) in dioxane (4 mL) was added HCl/dioxane (4 M, 54.09 μL) and the resulting mixture was stirred at 25° C. for 1 hr. LCMS showed a peak with desired mass. The mixture was concentrated in vacuum to afford 5-(4-((1-aminopiperidin-4-yl)methyl)piperazin-1-yl)-2-(2,6-dioxopiperidin-3-yl)isoindoline-1,3-dione (100 mg, crude, HCl salt) as a yellow solid. MS(M+H)⁺=455.2

### Step 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-((4-(2-(2,6-dioxopiperidin-3-yl)-1,3-dioxoisoindolin-5-yl)piperazin-1-yl)methyl)piperidin-1-yl)-3-methoxybenzamide (Compound 148)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (90 mg, 201.15 μmol) in DMF (2 mL) were added HATU (114.72 mg, 301.72 μmol) and DIPEA (77.99 mg, 603.44 μmol, 105.11 μL), then 5-(4-((1-aminopiperidin-4-yl)methyl)piperazin-1-yl)-2-(2,6-dioxopiperidin-3-yl)isoindoline-1,3-dione (98.76 mg, 201.15 μmol, HCl salt) was added and the resulting mixture was stirred at 25° C. for 16 hr. LCMS showed the starting material consumed completely and a peak with desired mass. The mixture was diluted with water (3 mL), extracted with EtOAc (5 mL×3). The organic layer was dried over Na₂SO₄, filtered and concentrated in vacuum. The crude product was purified by Prep-HPLC (column: Phenomenex Synergi Polar-RP 100*25 mm*4 μm; mobile phase: [water (TFA)-ACN]; B %: 30%-50%, 7 min) and Prep-HPLC (column: Waters Xbridge 150*25 mm*5 μm; mobile phase: [water(NH₄HCO₃)-ACN]; B %: 39%-69%, 10 min), the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-((4-(2-(2,6-dioxopiperidin-3-yl)-1,3-dioxoisoindolin-5-yl)piperazin-1-yl)methyl)piperidin-1-yl)-3-methoxybenzamide (15.8 mg, 17.14 μmol, 8.52% yield, 95.9% purity) as a yellow solid. MS(M+H)⁺=884.4.

¹H NMR (400 MHz, DMSO-d₆) δ=11.06 (s, 1H), 9.32 (s, 1H), 8.31-8.23 (m, 2H), 7.97 (s, 1H), 7.68 (d, J=8.44 Hz, 1H), 7.47-7.39 (m, 2H), 7.34 (s, 1H), 7.26 (d, J=7.09 Hz, 1H), 5.07 (dd, J=12.78, 5.44 Hz, 1H), 4.83-4.70 (m, 1H), 4.05 (t, J=14.06 Hz, 2H), 3.94 (s, 3H), 3.45 (s, 4H), 3.33 (s, 3H), 3.03 (d, J=9.66 Hz, 2H), 2.92-2.84 (m, 1H), 2.79 (t, J=10.33 Hz, 2H), 2.64-2.57 (m, 2H), 2.57-2.53 (m, 4H), 2.22 (d, J=6.48 Hz, 2H), 2.07-1.99 (m, 1H), 1.95 (s, 2H), 1.83-1.68 (m, 4H), 1.59 (d, J=1.10 Hz, 5H), 1.32-1.20 (m, 2H).

### Example 149. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(3-(2-(4-(2-(2,6-dioxopiperidin-3-yl)-1,3-dioxoisoindolin-5-yl)piperazin-1-yl)ethyl)pyrrolidin-1-yl)-3-methoxybenzamide (Compound 149)

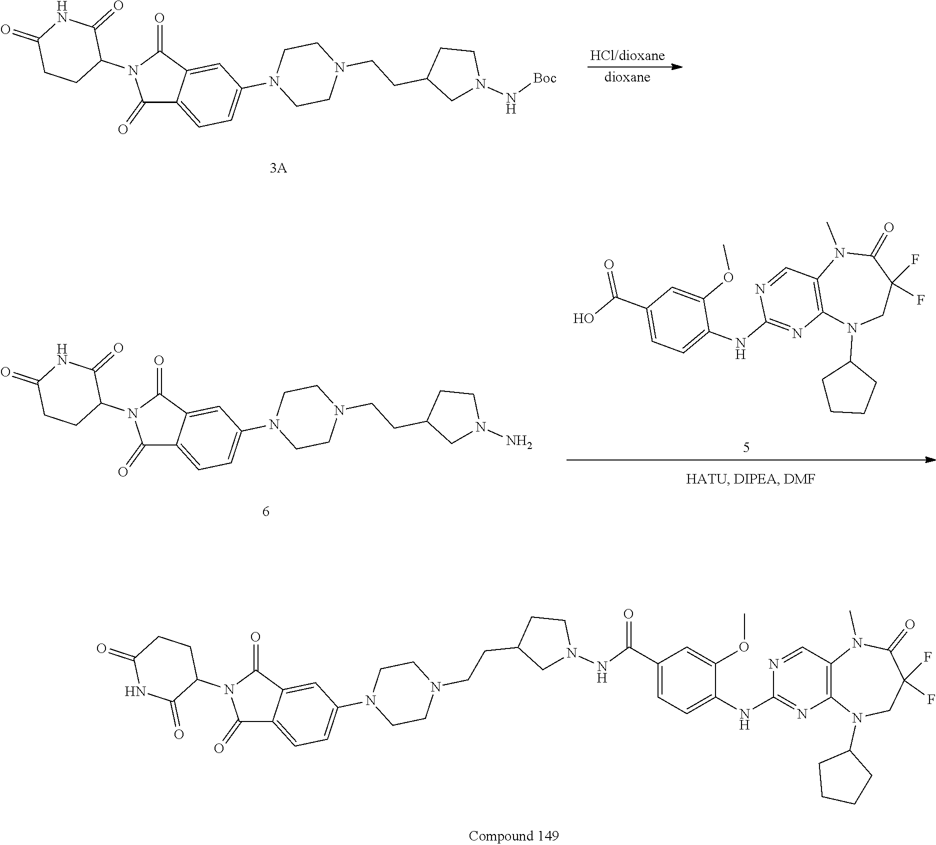 [SMILES: CC(C)(C)OC(=O)NN1CCC(CCN2CCN(c3ccc4c(c3)C(=O)N(C3CCC(=O)NC3=O)C4=O)CC2)C1 ; NN1CCC(CCN2CCN(c3ccc4c(c3)C(=O)N(C3CCC(=O)NC3=O)C4=O)CC2)C1 ; COc1cc(C(=O)O)ccc1Nc1ncc2c(n1)N(C1CCCC1)CC(F)(F)C(=O)N2C ; COc1cc(C(=O)NN2CCC(CCN3CCN(c4ccc5c(c4)C(=O)N(C4CCC(=O)NC4=O)C5=O)CC3)C2)ccc1Nc1ncc2c(n1)N(C1CCCC1)CC(F)(F)C(=O)N2C]

### Step 1. Synthesis of 5-(4-(2-(1-aminopyrrolidin-3-yl)ethyl)piperazin-1-yl)-2-(2,6-dioxopiperidin-3-yl)isoindoline-1,3-dione (6)

To a solution of tert-butyl (3-(2-(4-(2-(2,6-dioxopiperidin-3-yl)-1,3-dioxoisoindolin-5-yl)piperazin-1-yl)ethyl)pyrrolidin-1-yl)carbamate (240.00 mg, 432.72 μmol) in dioxane (4 mL) was added HCl/dioxane (4 M, 108.18 μL), the mixture was stirred at 25° C. for 1 hr. LCMS showed a peak with desired mass, the mixture was concentrated in vacuum to afford 5-(4-(2-(1-aminopyrrolidin-3-yl)ethyl)piperazin-1-yl)-2-(2,6-dioxopiperidin-3-yl)isoindoline-1,3-dione (200 mg, crude, HCl salt) as a yellow solid. MS(M+H)⁺=455.0

### Step 2. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(3-(2-(4-(2-(2,6-dioxopiperidin-3-yl)-1,3-dioxoisoindolin-5-yl)piperazin-1-yl)ethyl)pyrrolidin-1-yl)-3-methoxybenzamide (Compound 149)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (90 mg, 201.15 μmol) in DMF (2 mL) were added HATU (114.72 mg, 301.72 μmol) and DIPEA (77.99 mg, 603.44 μmol, 105.11 μL), then 5-(4-(2-(1-aminopyrrolidin-3-yl)ethyl)piperazin-1-yl)-2-(2,6-dioxopiperidin-3-yl)isoindoline-1,3-dione (98.76 mg, 201.15 μmol, HCl salt) was added and the reaction mixture was stirred at 25° C. for 16 hr. LCMS showed the starting material consumed completely, and a peak with desired mass. The reaction mixture was diluted with water (3 mL), extracted with EtOAc (5 mL×3). The organic layer was dried over Na₂SO₄, filtered and concentrated under vacuum. The crude product was purified by Prep-HPLC (column: Phenomenex Synergi Polar-RP 100*25 mm*4 μm; mobile phase: [water(TFA)-ACN]; B %: 30%-50%, 7 min) and Prep-HPLC (column: Waters Xbridge 150*25 mm*5 μm; mobile phase: [water(NH₄HCO₃)-ACN]; B %: 38%-68%, 10 min), the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(3-(2-(4-(2-(2,6-dioxopiperidin-3-yl)-1,3-dioxoisoindolin-5-yl)piperazin-1-yl)ethyl)pyrrolidin-1-yl)-3-methoxybenzamide (20.4 mg, 22.32 μmol, 11.09% yield, 96.7% purity) as yellow powder. MS(M+H)⁺=884.0.

¹H NMR (400 MHz, DMSO-d₆) δ=11.10 (s, 1H), 9.40 (s, 1H), 8.33-8.23 (m, 2H), 7.98 (s, 1H), 7.72 (d, J=8.38 Hz, 1H), 7.50-7.36 (m, 3H), 7.31 (d, J=8.25 Hz, 1H), 5.09 (dd, J=12.82, 5.32 Hz, 1H), 4.77 (t, J=7.82 Hz, 1H), 4.05 (t, J=13.95 Hz, 2H), 3.93 (s, 3H), 3.37-3.35 (m, 4H), 3.33 (s, 3H), 3.19-2.99 (m, 4H), 2.94-2.82 (m, 2H), 2.77-2.67 (m, 2H), 2.65-2.52 (m, 5H), 2.36-2.22 (m, J=14.57, 7.22 Hz, 1H), 2.07-1.89 (m, 4H), 1.78-1.39 (m, 9H).

### Example 150. Synthesis of 4-((7,7-difluoro-9-isopropyl-5-methyl-6-oxo-6,7,8,9-tetrahydro-5H-pyrimido[4,5-b ][1,4]diazepin-2-yl)amino)-N-((1r,4r)-4-(4-(2-(2,6-dioxopiperidin-3-yl)-1,3-dioxoisoindolin-5-yl)piperazin-1-yl)cyclohexyl)-3-methoxybenzamide (trans) (Compound 150)

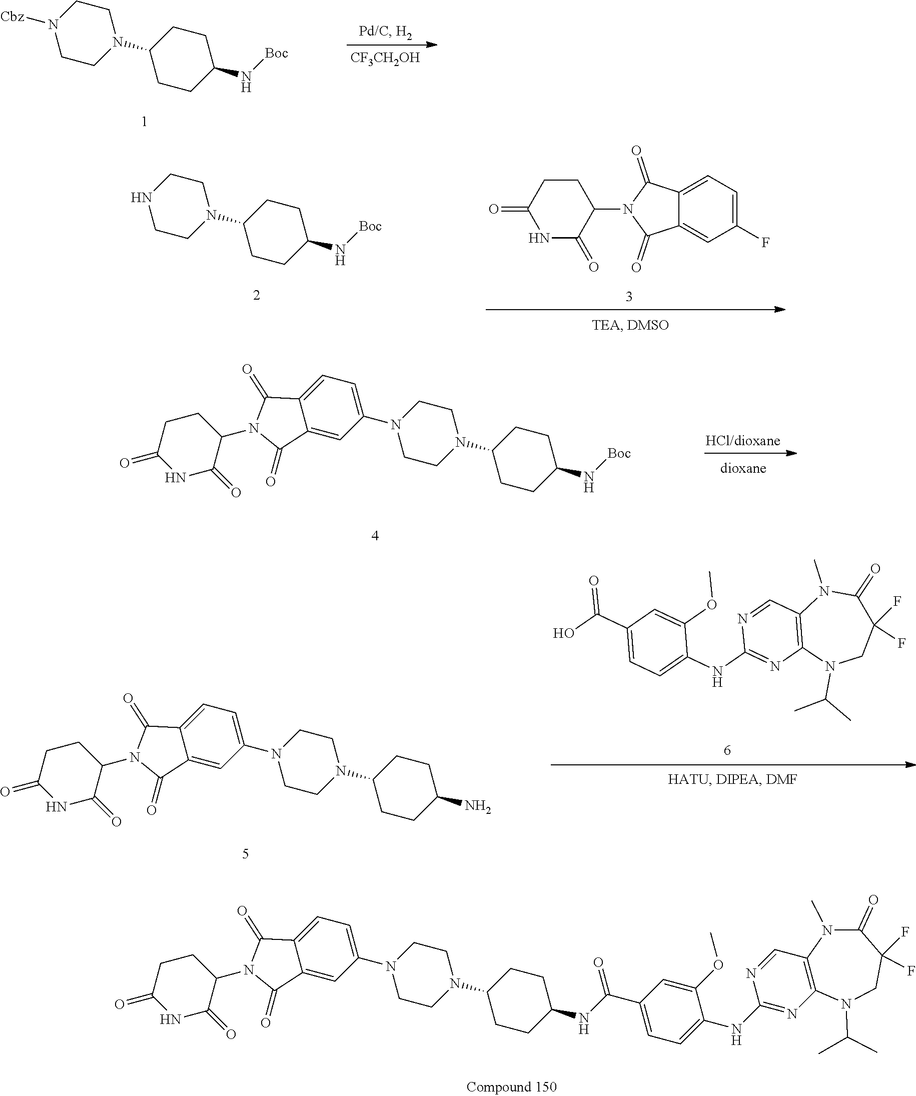

### Step 1. Synthesis of tert-butyl ((1r,4r)-4-(piperazin-1-yl)cyclohexyl)carbamate (trans) (2)

To a solution of benzyl 4-((1r,4r)-4-((tert-butoxycarbonyl)amino)cyclohexyl)piperazine-1-carboxylate (300 mg, 718.49 μmol) in CF₃CH₂OH (6 mL) was added Pd/C (0.1 g, 10% purity) under N₂ atmosphere. The suspension was degassed and purged with H₂ for 3 times. The mixture was stirred at 20° C. for 16 h under H₂ (15 Psi). LCMS showed starting material was consumed completely and a peak with desired mass. The reaction mixture was diluted with CF₃CH₂OH (30 mL) and filtered. The filtrate was concentrated to afford tert-butyl ((1r,4r)-4-(piperazin-1-yl)cyclohexyl)carbamate (trans) (170 mg, 599.84 μmol, 83.49% yield) as an off-white solid. MS(M+H)⁺=284.5

### Step 2. Synthesis of tert-butyl ((1r,4r)-4-(4-(2-(2,6-dioxopiperidin-3-yl)-1,3-dioxoisoindolin-5-yl)piperazin-1-yl)cyclohexyl)carbamate (trans) (4)

To a solution of 2-(2,6-dioxopiperidin-3-yl)-5-fluoroisoindoline-1,3-dione (150 mg, 543.05 μmol) in DMSO (4 mL) were added TEA (164.85 mg, 1.63 mmol, 226.76 μL) and tert-butyl ((1r,4r)-4-(piperazin-1-yl)cyclohexyl)carbamate (trans) (169.29 mg, 597.35 μmol) at 20° C. and the resulting mixture was stirred at 100° C. for 16 h. LCMS showed starting material was consumed completely and a peak (77%) with desired mass. The reaction mixture was diluted with H₂O (15 mL) and extracted with EtOAc (15 mL×3). The organic layer was washed with brine (15 mL×3), dried over Na₂SO₄, filtered and concentrated. The residue was purified by flash silica gel chromatography (12 g SepaFlash® Silica Flash Column, Eluent of 0˜100% EtOAc/Petroleum ether gradient @100 mL/min) to afford tert-butyl ((1r,4r)-4-(4-(2-(2,6-dioxopiperidin-3-yl)-1,3-dioxoisoindolin-5-yl)piperazin-1-yl)cyclohexyl)carbamate (trans) (272 mg, 499.02 μmol, 91.89% yield, 99% purity) as a yellow solid. MS(M+H)⁺=540.3

### Step 3. Synthesis of 5-(4-((1r,4r)-4-aminocyclohexyl)piperazin-1-yl)-2-(2,6-dioxopiperidin-3-yl)isoindoline-1,3-dione (trans) (5)

To a solution of tert-butyl ((1r,4r)-4-(4-(2-(2,6-dioxopiperidin-3-yl)-1,3-dioxoisoindolin-5-yl)piperazin-1-yl)cyclohexyl)carbamate (trans) (272 mg, 504.06 μmol) in dioxane (3 mL) was added HCl/dioxane (4 M, 12 mL) at 20° C. and the resulting mixture was stirred at 20° C. for 0.5 h. LCMS showed starting material was consumed completely and a peak (72%) with desired mass. The reaction mixture was concentrated in vacuum to afford 5-(4-((1r,4r)-4-aminocyclohexyl)piperazin-1-yl)-2-(2,6-dioxopiperidin-3-yl)isoindoline-1,3-dione (trans) (240 mg, crude, HCl salt) as a yellow solid. MS(M+H)⁺=440.4

### Step 4. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((1r,4r)-4-(4-(2-(2,6-dioxopiperidin-3-yl)-1,3-dioxoisoindolin-5-yl)piperazin-1-yl)cyclohexyl)-3-methoxybenzamide (trans) (Compound 150)

To a solution of 4-((7,7-difluoro-9-isopropyl-5-methyl-6-oxo-6,7,8,9-tetrahydro-5H-pyrimido[4,5-b][1, 4]diazepin-2-yl)amino)-3-methoxybenzoic acid (60 mg, 142.38 μmol) in DMF (1 mL) were added HATU (59.55 mg, 156.62 μmol) and DIPEA (36.80 mg, 284.77 μmol, 49.60 μL). The mixture was stirred at 20° C. for 10 min and a solution of 5-(4-((1r,4r)-4-aminocyclohexyl)piperazin-1-yl)-2-(2,6-dioxopiperidin-3-yl)isoindoline-1,3-dione (trans) (81.32 mg, 170.86 μmol, HCl salt) with DIPEA (36.80 mg, 284.77 μmol, 49.60 μL) in DMF (1 mL) was added and the resulting mixture was stirred at 20° C. for 1 h. LCMS showed starting material was consumed completely and a peak (61%) with desired mass. The reaction mixture was diluted with H₂O (10 mL) and extracted with EtOAc (10 mL×3). The organic layer was dried over Na₂SO₄, filtered and concentrated. The residue was purified by prep-HPLC (column: Phenomenex Synergi C18 150*25 mm*10 μm; mobile phase: [water(FA)-ACN]; B %: 11%-50%, 13 min) and the eluent was lyophilized to afford 4-((7,7-difluoro-9-isopropyl-5-methyl-6-oxo-6,7,8,9-tetrahydro-5H-pyrimido[4,5-b][1, 4]diazepin-2-yl)amino)-N-((1r,4r)-4-(4-(2-(2,6-dioxopiperidin-3-yl)-1,3-dioxoisoindolin-5-yl)piperazin-1-yl)cyclohexyl)-3-methoxybenzamide (trans) (72.8 mg, 79.44 μmol, 55.79% yield, 97% purity, FA) as a yellow solid. MS(M+H)⁺=843.1

¹H NMR (400 MHz, DMSO-d₆) δ=11.10 (s, 1H), 9.58-9.33 (m, 1H), 8.31 (d, J=8.3 Hz, 1H), 8.22 (s, 1H), 8.18-8.12 (m, 1H), 7.89 (s, 1H), 7.77 (br d, J=8.6 Hz, 1H), 7.54-7.44 (m, 2H), 7.38 (br d, J=8.9 Hz, 1H), 5.10 (dd, J=5.4, 12.9 Hz, 1H), 4.88 (td, J=6.7, 13.5 Hz, 1H), 4.40-4.16 (m, 2H), 4.04 (br t, J=13.4 Hz, 2H), 3.94 (s, 3H), 3.86-3.76 (m, 1H), 3.70-3.54 (m, 2H), 3.32 (s, 3H), 3.27-3.17 (m, 4H), 2.95-2.84 (m, 1H), 2.63-2.54 (m, 3H), 2.22-2.09 (m, 2H), 2.08-1.95 (m, 3H), 1.71-1.55 (m, 2H), 1.49-1.37 (m, 2H), 1.24 (d, J=6.7 Hz, 6H).

### Example 151. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(3-(2-(4-(2-(2,6-dioxopiperidin-3-yl)-1,3-dioxoisoindolin-4-yl)piperazin-1-yl)ethyl)pyrrolidin-1-yl)-3-methoxybenzamide (Compound 151)

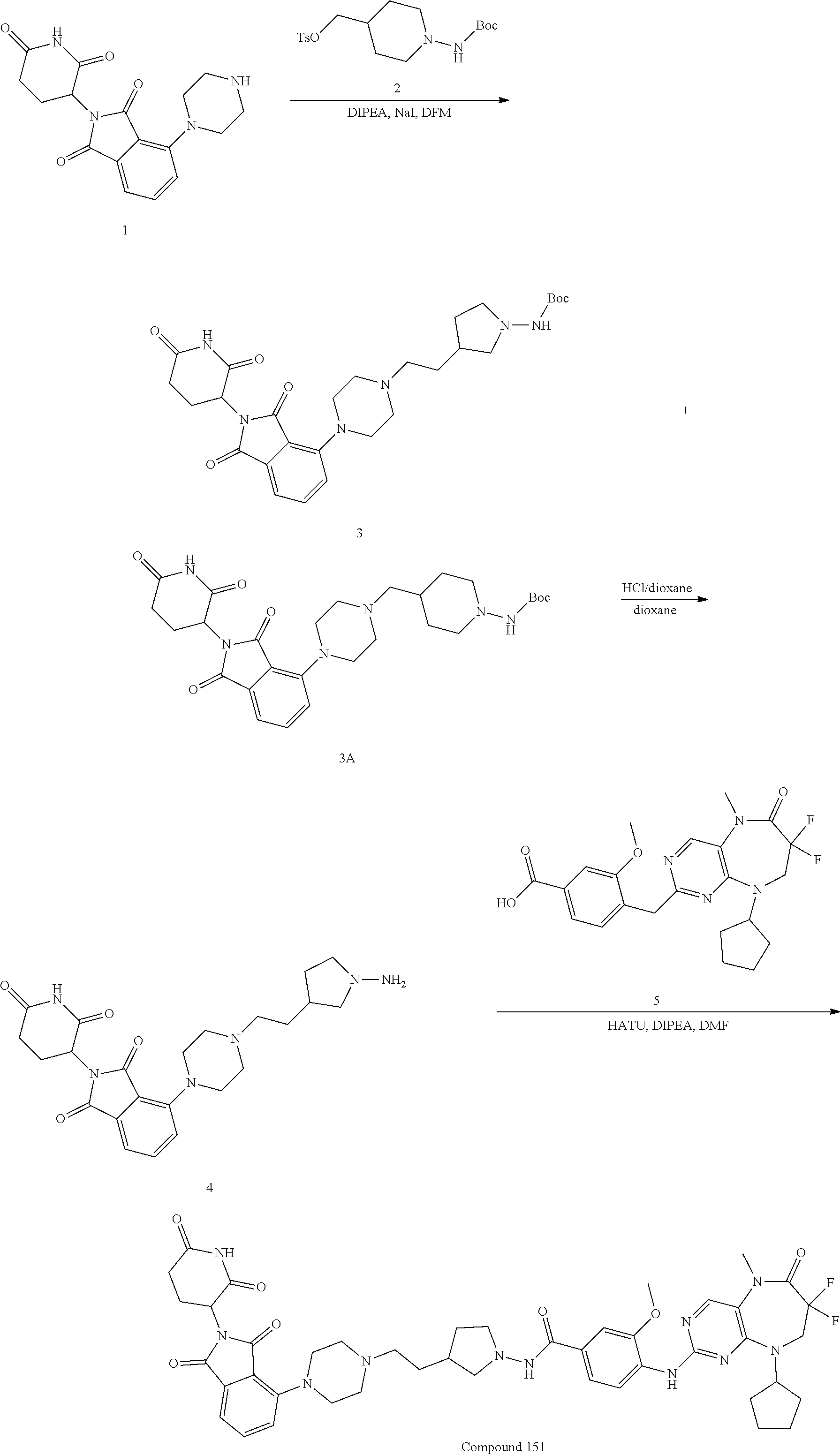

The compound 151 was synthesized by the method described in the scheme similar to the method described in Example 149.

MS(M+H)⁺=884.5, ¹H NMR (400 MHz, CDCl₃) δ=8.48 (d, J=8.2 Hz, 1H), 8.09-8.03 (m, 2H), 7.76 (s, 1H), 7.65-7.58 (m, 1H), 7.42 (br d, J=6.6 Hz, 2H), 7.24-7.22 (m, 1H), 7.18 (d, J=8.6 Hz, 1H), 6.96-6.84 (m, 1H), 5.01-4.94 (m, 1H), 4.87-4.79 (m, 1H), 3.99 (s, 3H), 3.90 (t, J=13.6 Hz, 2H), 3.41 (s, 3H), 3.39-3.34 (m, 4H), 3.31-3.24 (m, 1H), 3.06-2.98 (m, 1H), 2.95-2.84 (m, 2H), 2.83-2.63 (m, 7H), 2.51-2.32 (m, 3H), 2.21-2.03 (m, 5H), 1.84-1.63 (m, 8H).

### Example 152. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((1r,4r)-4-(((1-(2-(2,6-dioxopiperidin-3-yl)-1,3-dioxoisoindolin-4-yl)piperidin-4-yl)methyl)(methyl)amino)cyclohexyl)-3-methoxybenzamide (trans) (Compound 152)

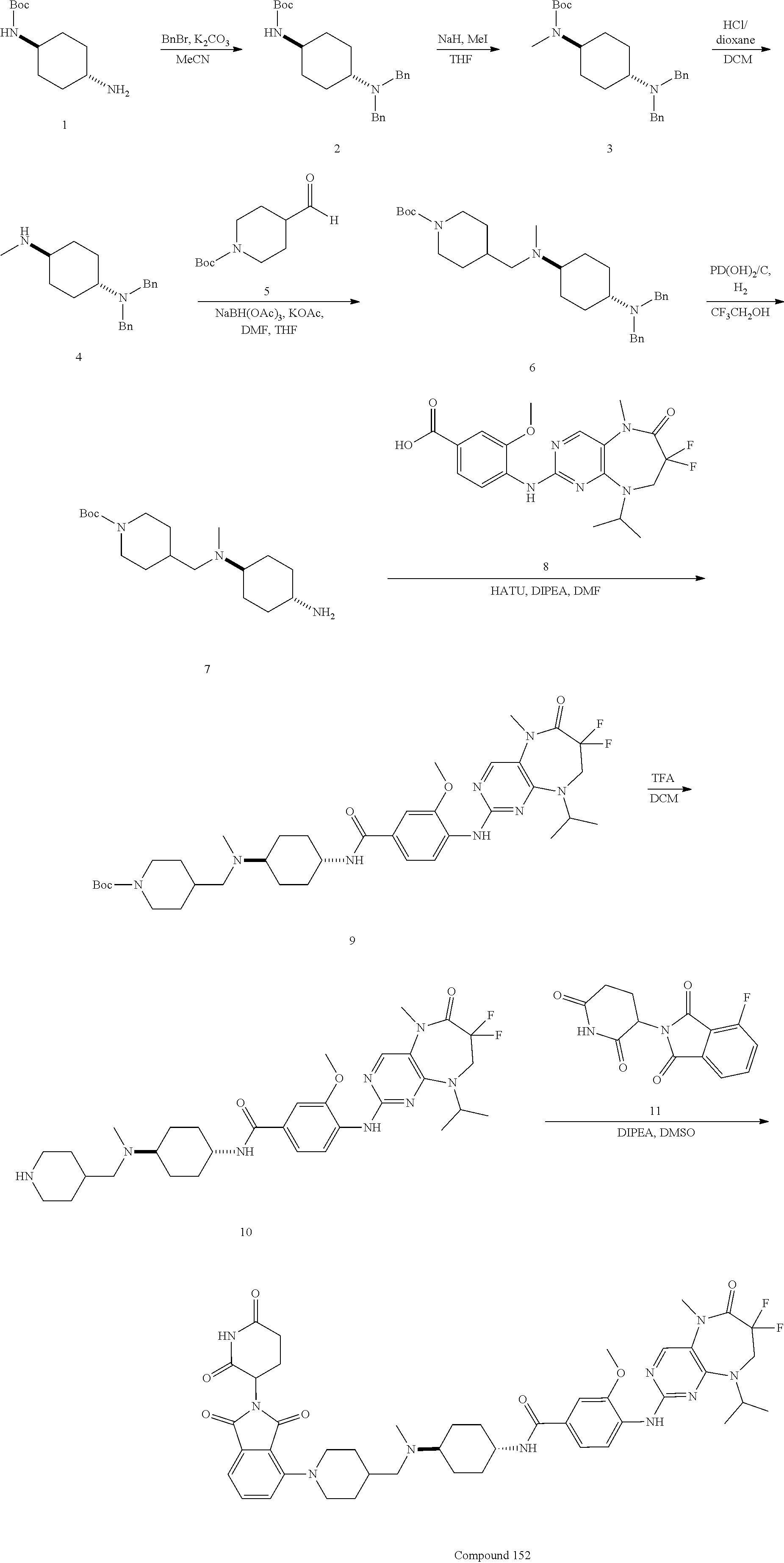

### Step 1. Synthesis of tert-butyl ((1r,4r)-4-(dibenzylamino)cyclohexyl)carbamate (trans) (2)

To a solution of tert-butyl ((1r,4r)-4-aminocyclohexyl)carbamate (trans) (25.2 g, 117.59 mmol) in MeCN (500 mL) were added K₂CO₃ (65.01 g, 470.36 mmol) and BnBr (42.64 g, 249.29 mmol, 29.61 mL), the mixture was stirred at 60° C. for 16 hours. TLC (SiO₂, Petroleum ether:EtOAc=5:1) indicated the starting material was consumed completely and one major new spot with lower polarity was detected. The reaction mixture was diluted with H₂O (500 mL) at 0° C., and extracted with EtOAc (500 mL×3) at 0° C. The combined organic layers were dried over Na₂SO₄, filtered and concentrated in vacuum. The residue was triturated with Petroleum ether (80 mL) for 10 minutes, the suspension was filtered and the filter cake was washed with Petroleum ether (50 mL). The filter cake was collected and dried to afford tert-butyl ((1r,4r)-4-(dibenzylamino)cyclohexyl)carbamate (trans) (44.06 g, 111.67 mmol, 94.97% yield) as a white solid. MS(M+H)⁺=395.2

### Step 2. Synthesis of tert-butyl ((1r,4r)-4-(dibenzylamino)cyclohexyl)(methyl)carbamate (trans) (3)

To a solution of tert-butyl ((1r,4r)-4-(dibenzylamino)cyclohexyl)carbamate (trans) (44.06 g, 111.67 mmol) in THF (400 mL) was added NaH (6.70 g, 167.51 mmol, 60% purity) slowly at 5° C. under N₂ atmosphere, the suspension was stirred at 5° C. for 30 minutes, then MeI (19.02 g, 134.01 mmol, 8.34 mL) was added dropwise at 5° C., the resulting mixture was stirred at 15° C. for 2.5 hours. TLC (SiO₂, Petroleum ether:EtOAc=5:1) indicated the starting material was consumed completely and one major new spot with lower polarity was detected. The mixture was poured into ice water (300 mL) slowly, to the mixture was added HCl solution (1 M) slowly at 0° C. to adjust pH=8˜9, the resulting mixture was extracted with EtOAc (400 mL×3). The combined organic layers were washed with brine (500 mL), dried over Na₂SO₄, filtered and concentrated in vacuum to afford tert-butyl ((1r,4r)-4-(dibenzylamino)cyclohexyl)(methyl)carbamate (trans) (48.5 g) as a light brown solid, which was used directly. MS(M+H)⁺=409.2

### Step 3. Synthesis of (1r,4r)-N1,N1-dibenzyl-N4-methylcyclohexane-1,4-diamine (trans) (4)

To a solution of tert-butyl ((1r,4r)-4-(dibenzylamino)cyclohexyl)(methyl)carbamate (trans) (48.5 g, 118.71 mmol) in DCM (100 mL) was added HCl/dioxane (4 M, 148.35 mL), the mixture was stirred at 15° C. for 12 hours. LCMS showed the starting material was consumed completely and a major peak (99%) with desired mass. The reaction mixture was concentrated in vacuum. The residue was triturated with MTBE (60 mL) for 10 minutes, the suspension was filtered and the filter cake was washed with MTBE (40 mL). The filter cake was collected and dried in vacuum to afford (1r,4r)-N1,N1-dibenzyl-N4-methylcyclohexane-1,4-diamine (trans) (43.8 g, 2HCl salt) as a light yellow solid. MS(M+H)⁺=309.2

### Step 4. Synthesis of tert-butyl 4-((((1r,4r)-4-(dibenzylamino)cyclohexyl)(methyl)amino)methyl) piperidine-1-carboxylate (trans) (6)

To a solution of (1r,4r)-N1,N1-dibenzyl-N4-methylcyclohexane-1,4-diamine (trans) (6 g, 15.73 mmol, 2HCl salt) and KOAc (3.86 g, 39.33 mmol) in DMF (30 mL) and THF (150 mL) was added tert-butyl 4-formylpiperidine-1-carboxylate (3.36 g, 15.73 mmol) at 0° C., the mixture was stirred at 0° C. for 30 minutes, then NaBH(OAc)₃ (6.67 g, 31.46 mmol) was added, the resulting mixture was stirred at 10° C. for 14 hours. LCMS showed the starting material was consumed completely and a major peak (96%) with desired mass. The reaction mixture was concentrated in vacuum to remove most of the solvent, the residue was diluted with H₂O (300 mL) and extracted with DCM (150 mL×3). The combined organic layers were dried over Na₂SO₄, filtered and concentrated under reduced pressure to afford tert-butyl 4-((((1r,4r)-4-(dibenzylamino)cyclohexyl)(methyl)amino)methyl)piperidine-1-carboxylate (trans) (8.3 g) as an off-white solid, which was used in the next step directly. MS(M+H)⁺=506.3

### Step 5. Synthesis of tert-butyl 4-((((1r,4r)-4-aminocyclohexyl)(methyl)amino)methyl)piperidine-1-carboxylate (trans) (7)

To a solution of tert-butyl 4-((((1r,4r)-4-(dibenzylamino)cyclohexyl)(methyl)amino)methyl)piperidine-1-carboxylate (trans) (8.3 g, 16.41 mmol) in CF₃CH₂OH (100 mL) was added Pd(OH)₂/C (1 g, 10% purity) under N₂ atmosphere, the mixture was degassed and purged with H₂ for 3 times, and the suspension was stirred at 15° C. for 24 hours under H₂ (15 psi) atmosphere. LCMS showed the starting material was consumed completely and 44% of tert-butyl 4-((((1r,4r)-4-(benzylamino)cyclohexyl)(methyl)amino)methyl)piperidine-1-carboxylate remained, the reaction mixture was stirred at 15° C. for further 32 hours under H₂ (15 psi) atmosphere. LCMS showed the starting material was consumed. The mixture was filtered and the filter cake was washed with CF₃CH₂OH (100 mL), the filtrate was concentrated in vacuum. The residue was triturated with MTBE (20 mL) for 20 minutes, the suspension was filtered and the filter cake was washed with MTBE (20 mL), the filtrate was concentrated in vacuum to afford tert-butyl 4-((((1r,4r)-4-aminocyclohexyl)(methyl)amino)methyl)piperidine-1-carboxylate (trans) (3.45 g, 10.60 mmol, 64.58% yield) as a pink solid. MS(M+H)⁺=326.2

### Step 6. Synthesis of tert-butyl 4-((((1r,4r)-4-(4-((7,7-difluoro-9-isopropyl-5-methyl-6-oxo-6,7,8,9-tetrahydro-5H-pyrimido[4,5-b][1,4]diazepin-2-yl)amino)-3-methoxybenzamido)cyclohexyl)(methyl)amino)methyl)piperidine-1-carboxylate (trans) (9)

To a solution of 4-((7,7-difluoro-9-isopropyl-5-methyl-6-oxo-6,7,8,9-tetrahydro-5H-pyrimido[4,5-b][1, 4]diazepin-2-yl)amino)-3-methoxybenzoic acid (500 mg, 1.19 mmol) in DMF (10 mL) were added HATU (586.50 mg, 1.54 mmol) and DIPEA (306.70 mg, 2.37 mmol, 413.34 μL), the mixture was stirred at 15° C. for 15 minutes, then tert-butyl 4-((((1r,4r)-4-aminocyclohexyl)(methyl)amino)methyl)piperidine-1-carboxylate (trans) (386.20 mg, 1.19 mmol) was added, the resulting mixture was stirred at 15° C. for 1 hour. LCMS showed a peak (80%) with desired mass. The residue was diluted with H₂O (100 mL) and extracted with EtOAc (50 mL×3). The combined organic layers were washed with brine (100 mL×3), dried over Na₂SO₄, filtered and concentrated in vacuum. The residue was purified by flash silica gel chromatography (12 g SepaFlash® Silica Flash Column, Eluent of 100% EtOAc to 10% DCM/methanol gradient @80 mL/min) to afford tert-butyl 4-((((1r,4r)-4-(4-((7,7-difluoro-9-isopropyl-5-methyl-6-oxo-6,7,8,9-tetrahydro-5H-pyrimido[4,5-b][1,4]diazepin-2-yl)amino)-3-methoxybenzamido)cyclohexyl)(methyl)amino)methyl)piperidine-1-carboxylate (trans) (420 mg, 576.23 μmol, 48.56% yield) as a brown solid. MS(M+H)⁺=729.3

### Step 7. Synthesis of 4-((7,7-difluoro-9-isopropyl-5-methyl-6-oxo-6,7,8,9-tetrahydro-5H-pyrimido[4,5-b][1,4]diazepin-2-yl)amino)-3-methoxy-N-((1r,4r)-4-(methyl(piperidin-4-ylmethyl)amino)cyclohexyl)benzamide (trans) (10)

To a solution of tert-butyl 4-((((1r,4r)-4-(4-((7,7-difluoro-9-isopropyl-5-methyl-6-oxo-6,7,8,9-tetrahydro-5H-pyrimido[4,5-b][1,4]diazepin-2-yl)amino)-3-methoxybenzamido)cyclohexyl)(methyl)amino)methyl)piperidine-1-carboxylate (trans) (160 mg, 219.52 μmol) in DCM (2 mL) was added TFA (250.30 mg, 2.20 mmol, 162.53 μL), the mixture was stirred at 15° C. for 3 hours. LCMS showed the starting material was consumed completely and a peak (83%) with desired mass. The reaction mixture was concentrated in vacuum to afford 4-((7,7-difluoro-9-isopropyl-5-methyl-6-oxo-6,7,8,9-tetrahydro-5H-pyrimido[4,5-b][1, 4]diazepin-2-yl)amino)-3-methoxy-N-((1r,4r)-4-(methyl(piperidin-4-ylmethyl)amino)cyclohexyl)benzamide (trans) (170 mg, TFA salt) as a brown gum, which was used the next step directly. MS(M+H)⁺=629.4

### Step 8. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((1r,4r)-4-(((1-(2-(2,6-dioxopiperidin-3-yl)-1,3-dioxoisoindolin-4-yl)piperidin-4-yl)methyl)(methyl)amino)cyclohexyl)-3-methoxybenzamide (trans) (Compound 152)

A mixture of 4-((7,7-difluoro-9-isopropyl-5-methyl-6-oxo-6,7,8,9-tetrahydro-5H-pyrimido[4,5-b][1, 4]diazepin-2-yl)amino)-3-methoxy-N-((1r,4r)-4-(methyl(piperidin-4-ylmethyl)amino)cyclohexyl)benzamide (trans) (85 mg, 114.44 μmol, TFA salt), 2-(2,6-dioxopiperidin-3-yl)-4-fluoroisoindoline-1,3-dione (41.09 mg, 148.77 μmol) and DIPEA (147.90 mg, 1.14 mmol, 199.33 μL) in DMSO (1.5 mL) was stirred at 100° C. for 16 hours. LCMS showed a peak (75%) with desired mass. To the mixture was added CH₃COOH to adjust pH<7. The resulting mixture was purified by prep-HPLC (column: Phenomenex C18 75*30 mm*3 μn; mobile phase: [water(FA)-ACN]; B %: 18%-48%, 2 min) followed by prep-HPLC (column: Waters Xbridge 150*25 mm*5 μm; mobile phase: [water(NH₄HCO₃)-ACN]; B %: 48%-78%, 8 min), the eluent was freeze-dried to afford 4-((7,7-difluoro-9-isopropyl-5-methyl-6-oxo-6,7,8,9-tetrahydro-5H-pyrimido[4,5-b][1, 4]diazepin-2-yl)amino)-N-((1r,4r)-4-(((1-(2-(2,6-dioxopiperidin-3-yl)-1,3-dioxoisoindolin-4-yl)piperidin-4-yl)methyl)(methyl)amino)cyclohexyl)-3-methoxybenzamide (trans) (20.2 mg, 21.68 μmol, 18.95% yield, 95% purity) as a yellow solid. MS(M+H)⁺=885.3.

¹H NMR (400 MHz, DMSO-d₆) δ=11.08 (br s, 1H), 8.35-8.25 (m, 1H), 8.22 (d, J=1.6 Hz, 1H), 8.05 (br d, J=6.9 Hz, 1H), 7.87 (s, 1H), 7.73-7.61 (m, 1H), 7.56-7.43 (m, 2H), 7.32 (br t, J=7.6 Hz, 2H), 5.15-5.02 (m, 1H), 4.95-4.80 (m, 1H), 4.03 (br t, J=13.4 Hz, 2H), 3.93 (s, 3H), 3.80-3.61 (m, 3H), 3.29 (s, 3H), 2.87 (br t, J=11.1 Hz, 3H), 2.64-2.56 (m, 2H), 2.34-2.25 (m, 3H), 2.22 (s, 3H), 2.07-1.98 (m, 1H), 1.97-1.88 (m, 2H), 1.86-1.72 (m, 4H), 1.66-1.55 (m, 1H), 1.36 (br s, 4H), 1.29 (br d, J=11.5 Hz, 2H), 1.24 (br d, J=6.5 Hz, 6H).

### Example 153. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((1r,4r)-4-(((1-(2-(2,6-dioxopiperidin-3-yl)-1,3-dioxoisoindolin-5-yl)piperidin-4-yl)methyl)(methyl)amino)cyclohexyl)-3-methoxybenzamide (trans) (Compound 153)

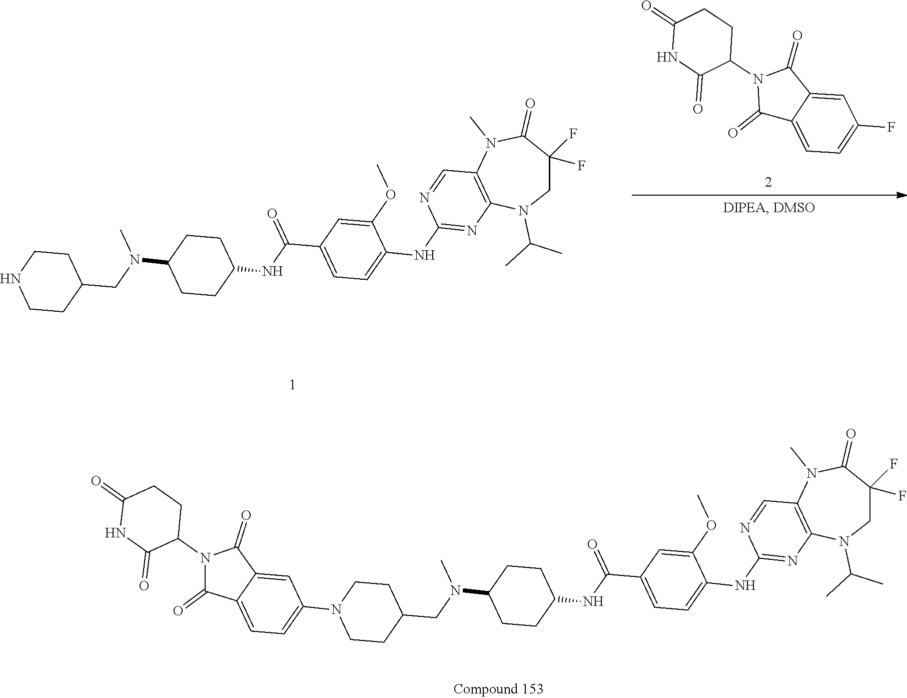

The compound 153 was synthesized by the method described in the scheme similar to the method described in Example 152.

MS(M+H)⁺=885.3, ¹H NMR (400 MHz, DMSO-d₆) δ=11.07 (s, 1H), 8.35-8.26 (m, 1H), 8.21 (s, 1H), 8.10-7.99 (m, 1H), 7.86 (s, 1H), 7.65 (d, J=8.5 Hz, 1H), 7.56-7.41 (m, 2H), 7.30 (s, 1H), 7.26-7.17 (m, 1H), 5.06 (dd, J=5.4, 12.8 Hz, 1H), 4.95-4.79 (m, 1H), 4.15-3.97 (m, 4H), 3.93 (s, 3H), 3.79-3.63 (m, 1H), 3.30 (s, 3H), 2.97 (t, J=12.1 Hz, 2H), 2.92-2.81 (m, 1H), 2.62-2.59 (m, 2H), 2.33-2.28 (m, 1H), 2.24 (d, J=6.4 Hz, 2H), 2.20 (s, 3H), 2.06-1.97 (m, 1H), 1.92-1.83 (m, 2H), 1.85-1.62 (m, 5H), 1.40-1.30 (m, 4H), 1.24 (d, J=6.6 Hz, 6H), 1.19-1.06 (m, 2H).

### Example 154. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(3-(4-(2-(2,6-dioxopiperidin-3-yl)-1,3-dioxoisoindolin-5-yl)piperazin-1-yl)pyrrolidin-1-yl)-3-methoxybenzamide (Compound 154)

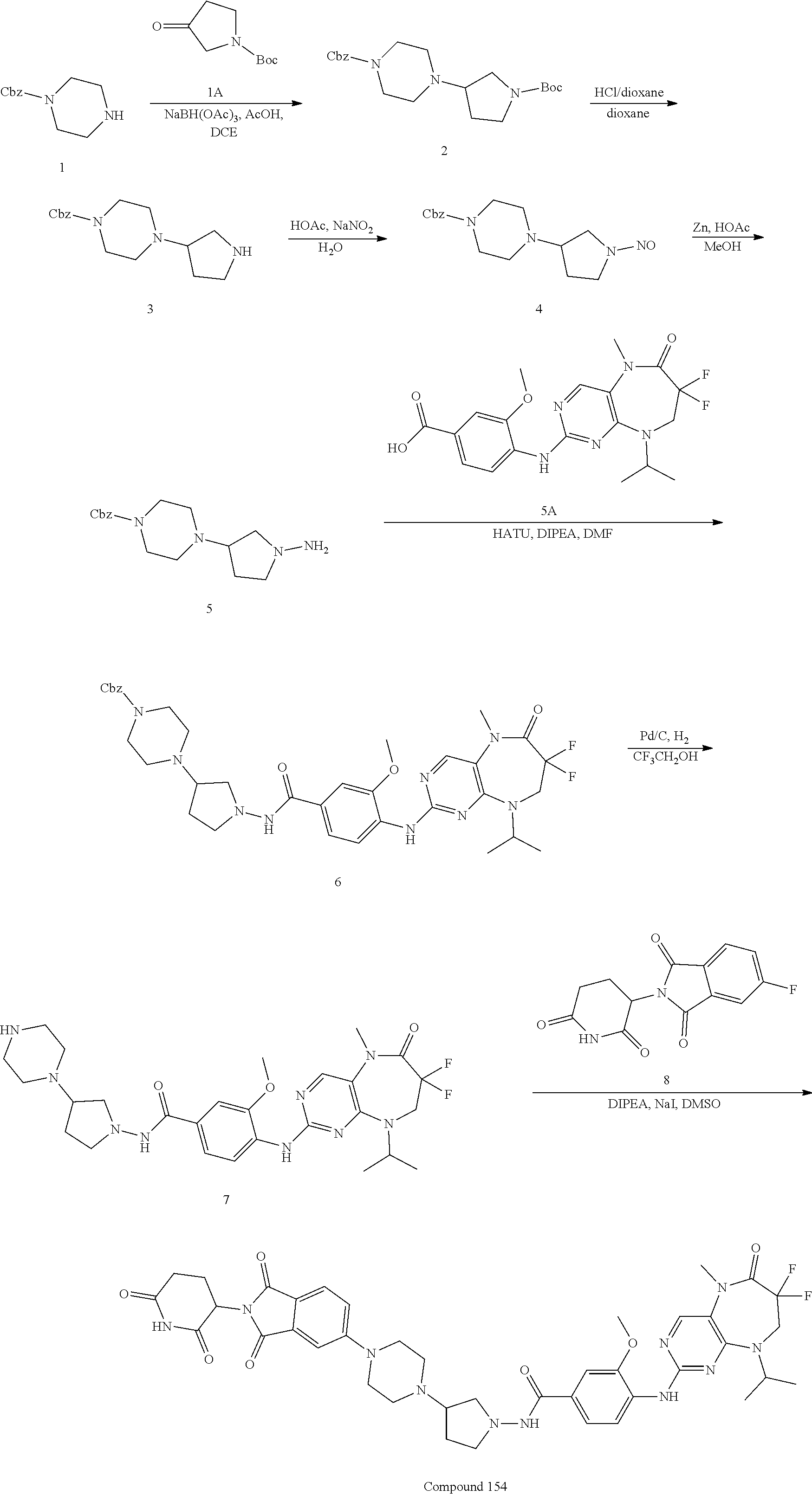

### Step 1. Synthesis of benzyl 4-(1-(tert-butoxycarbonyl)pyrrolidin-3-yl)piperazine-1-carboxylate (2)

To a solution of tert-butyl 3-oxopyrrolidine-1-carboxylate (5 g, 26.99 mmol) and benzyl piperazine-1-carboxylate (5.95 g, 26.99 mmol, 5.22 mL) in DCE (50 mL) was added AcOH (162.11 mg, 2.70 mmol, 154.39 μL), the mixture was stirred at 20° C. for 0.5 h, then NaBH(OAc)₃ (17.16 g, 80.98 mmol) was added and the resulting mixture was stirred at 20° C. for 16 h. LCMS showed desired mass, the mixture was diluted with water (50 mL), extracted with EtOAc (30 mL×3). The organic layer was dried over Na₂SO₄, filtered and the filtrate was concentrated under vacuum to afford benzyl 4-(1-(tert-butoxycarbonyl)pyrrolidin-3-yl)piperazine-1-carboxylate (9 g, crude) as yellow oil, used directly. MS(M+H)⁺=390.3

### Step 2. Synthesis of benzyl 4-(pyrrolidin-3-yl)piperazine-1-carboxylate (3)

To a solution of benzyl 4-(1-(tert-butoxycarbonyl)pyrrolidin-3-yl)piperazine-1-carboxylate (9 g, 23.11 mmol) in dioxane (10 mL) was added HCl/dioxane (4 M, 8 mL), the mixture was stirred at 20° C. for 2 hr. LCMS showed desired mass, the mixture was filtered. The filter cake was dried in vacuum to afford benzyl 4-(pyrrolidin-3-yl)piperazine-1-carboxylate (6.3 g, crude, HCl salt) as a yellow solid. MS(M+H)⁺=290.1

### Step 3. Synthesis of benzyl 4-(1-nitrosopyrrolidin-3-yl)piperazine-1-carboxylate (4)

To a solution of benzyl 4-(pyrrolidin-3-yl)piperazine-1-carboxylate (6.3 g, 19.34 mmol, HCl salt) in H₂O (50 mL) was added AcOH (56.70 g, 944.18 mmol, 54.00 mL) and NaNO₂ (1.33 g, 19.34 mmol), the mixture was stirred at 15° C. for 16 h. LCMS showed desired mass, the mixture was diluted with water (50 mL), and adjusted the pH to 7-8 with NaHCO₃, then extracted with EtOAc (50 mL×3). The organic layer was dried over Na₂SO₄, filtered, the filtrate was concentrated under vacuum to afford benzyl 4-(1-nitrosopyrrolidin-3-yl)piperazine-1-carboxylate (1.8 g, crude) as yellow oil. MS(M+H)⁺=319.1

### Step 4. Synthesis of benzyl 4-(1-aminopyrrolidin-3-yl)piperazine-1-carboxylate (5)

To a solution of benzyl 4-(1-nitrosopyrrolidin-3-yl)piperazine-1-carboxylate (0.9 g, 2.83 mmol) in MeOH (10 mL) were added Zn (1.11 g, 16.96 mmol) and AcOH (9.45 g, 157.36 mmol, 9.00 mL) at −10° C., after addition, the suspension was warmed to 15° C. and stirred at 15° C. for 2 h. LCMS showed the starting material consumed completely, and a peak with desired mass. The reaction mixture was filtered, the filtrate was concentrated under vacuum to afford benzyl 4-(1-aminopyrrolidin-3-yl)piperazine-1-carboxylate (500 mg, crude) as brown oil. MS(M+H)⁺=305.1

### Step 5. Synthesis of benzyl 4-(1-(4-((7,7-difluoro-9-isopropyl-5-methyl-6-oxo-6,7,8,9-tetrahydro-5H-pyrimido[4,5-b][1,4]diazepin-2-yl)amino)-3-methoxybenzamido)pyrrolidin-3-yl)piperazine-1-carboxylate (6)

To a solution of 4-((7,7-difluoro-9-isopropyl-5-methyl-6-oxo-6,7,8,9-tetrahydro-5H-pyrimido[4,5-b][1, 4]diazepin-2-yl)amino)-3-methoxybenzoic acid (320 mg, 759.38 μmol) in DMF (6 mL) were added HATU (433.11 mg, 1.14 mmol) and DIPEA (294.43 mg, 2.28 mmol, 396.81 μL), then benzyl 4-(1-aminopyrrolidin-3-yl)piperazine-1-carboxylate (300.49 mg, 987.19 μmol) was added and the resulting mixture was stirred at 25° C. for 16 hr. LCMS showed starting material consumed completely and desired product was detected. The reaction mixture was diluted with water (3 mL), and extracted with EtOAc (5 mL×3). The organic layer was dried over Na₂SO₄, filtered and concentrated under vacuum. The crude product was purified by flash silica gel chromatography (Biotage, 4 g SepaFlash® Silica Flash Column, Eluent of 10% MeOH/DCM gradient @20 mL/min) to afford benzyl 4-(1-(4-((7,7-difluoro-9-isopropyl-5-methyl-6-oxo-6,7,8,9-tetrahydro-5H-pyrimido[4,5-b][1,4]diazepin-2-yl)amino)-3-methoxybenzamido)pyrrolidin-3-yl)piperazine-1-carboxylate (380 mg, 504.68 μmol, 66.46% yield, 94% purity) as a brown powder. MS(M+H)⁺=708.4

### Step 6. Synthesis of 4-((7,7-difluoro-9-isopropyl-5-methyl-6-oxo-6,7,8,9-tetrahydro-5H-pyrimido[4,5-b][1,4]diazepin-2-yl)amino)-3-methoxy-N-(3-(piperazin-1-yl)pyrrolidin-1-yl)benzamide (7)

To a solution of benzyl 4-(1-(4-((7,7-difluoro-9-isopropyl-5-methyl-6-oxo-6,7,8,9-tetrahydro-5H-pyrimido[4,5-b][1,4]diazepin-2-yl)amino)-3-methoxybenzamido)pyrrolidin-3-yl)piperazine-1-carboxylate (280 mg, 395.61 μmol) in CF₃CH₂OH (12 mL) was added Pd/C (419.35 mg, 395.61 μmol, 10% purity) under N₂ atmosphere, the mixture was degassed and purged with H₂ for 3 times. The mixture was stirred at 20° C. for 16 hr under H₂ (15 psi). LCMS showed starting material consumed completely, and a peak with desired mass. The mixture was filtered, the filtrate was concentrated under vacuum to afford 4-((7,7-difluoro-9-isopropyl-5-methyl-6-oxo-6,7,8,9-tetrahydro-5H-pyrimido[4,5-b][1, 4]diazepin-2-yl)amino)-3-methoxy-N-(3-(piperazin-1-yl)pyrrolidin-1-yl)benzamide (250 mg, crude) as yellow oil. MS(M+H)⁺=574.4

### Step 7. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(3-(4-(2-(2,6-dioxopiperidin-3-yl)-1,3-dioxoisoindolin-5-yl)piperazin-1-yl)pyrrolidin-1-yl)-3-methoxybenzamide (Compound 154)

To a solution of 4-((7,7-difluoro-9-isopropyl-5-methyl-6-oxo-6,7,8,9-tetrahydro-5H-pyrimido[4,5-b][1, 4]diazepin-2-yl)amino)-3-methoxy-N-(3-(piperazin-1-yl)pyrrolidin-1-yl)benzamide (80 mg, 139.46 μmol) and 2-(2,6-dioxopiperidin-3-yl)-5-fluoroisoindoline-1,3-dione (46.23 mg, 167.35 μmol) in DMSO (2 mL) were added DIPEA (54.07 mg, 418.38 μmol, 72.87 μL) and NaI (4.18 mg, 27.89 μmol), the mixture was stirred at 90° C. for 16 hr. LCMS showed the starting material consumed completely, a peak with desired mass. The mixture was diluted with water (3 mL), and extracted with EtOAc (5 mL×3). The organic layer was dried over Na₂SO₄, filtered and concentrated under vacuum. The crude product was purified by flash silica gel chromatography (Biotage, 4 g SepaFlash® Silica Flash Column, Eluent of 4˜15% MeOH/EtOAc gradient @20 mL/min) followed by Prep-HPLC (column: Waters Xbridge 150*25 mm*5 μm; mobile phase: [water(NH₄HCO₃)-ACN]; B %: 32%-62%, 10 min), the eluent was lyophilized to afford 4-((7,7-difluoro-9-isopropyl-5-methyl-6-oxo-6,7,8,9-tetrahydro-5H-pyrimido[4,5-b][1, 4]diazepin-2-yl)amino)-N-(3-(4-(2-(2,6-dioxopiperidin-3-yl)-1,3-dioxoisoindolin-5-yl) piperazin-1-yl)pyrrolidin-1-yl)-3-methoxybenzamide (7.6 mg, 8.98 μmol, 6.44% yield, 98.0% purity) as a yellow solid. MS(M+H)⁺=830.4.

¹H NMR (400 MHz, DMSO-d₆) δ=11.08 (s, 1H), 9.37 (s, 1H), 8.32 (d, J=8.50 Hz, 1H), 8.23 (s, 1H), 7.89 (s, 1H), 7.70 (d, J=8.50 Hz, 1H), 7.48-7.42 (m, 2H), 7.36 (s, 1H), 7.28 (d, J=8.63 Hz, 1H), 5.08 (dd, J=12.82, 5.44 Hz, 1H), 4.92-4.88 (m, 1H), 4.05 (t, J=13.63 Hz, 2H), 3.94 (s, 3H), 3.55-3.46 (m, 4H), 3.31 (s, 3H), 3.20-3.09 (m, 2H), 3.09-2.98 (m, 3H), 2.98-2.82 (m, 2H), 2.66-2.54 (m, 5H), 2.07-1.96 (m, 2H), 1.83-1.74 (m, 1H), 1.25 (d, J=6.75 Hz, 6H).

### Example 155. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(3-(4-(2-(2,6-dioxopiperidin-3-yl)-1,3-dioxoisoindolin-4-yl)piperazin-1-yl)pyrrolidin-1-yl)-3-methoxybenzamide (Compound 155)

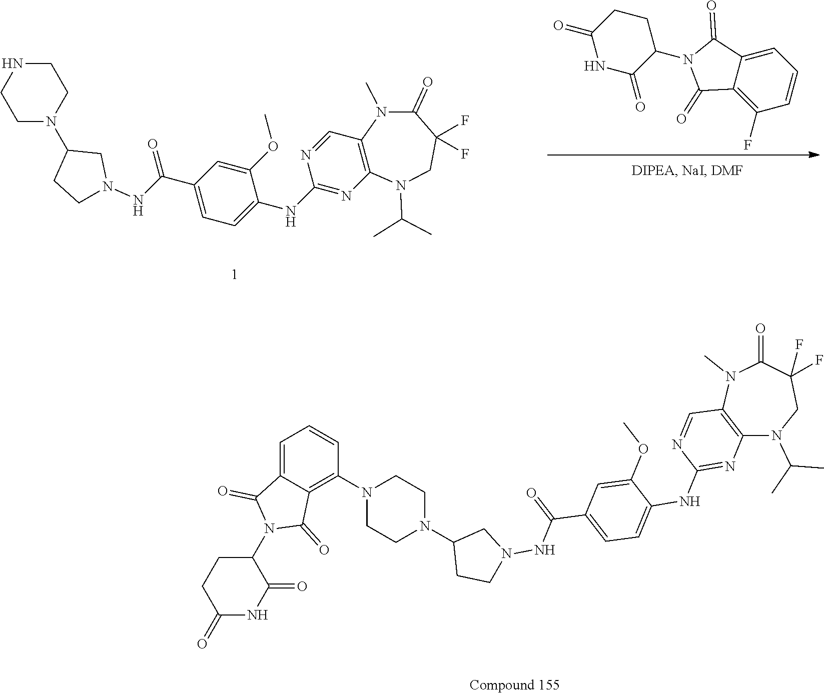 [SMILES: COc1cc(C(=O)NN2CCC(N3CCNCC3)C2)ccc1Nc1ncc2c(n1)N(C(C)C)CC(F)(F)C(=O)N2C ; O=C1CCC(N2C(=O)c3cccc(F)c3C2=O)C(=O)N1 ; COc1cc(C(=O)NN2CCC(N3CCN(c4cccc5c4C(=O)N(C4CCC(=O)NC4=O)C5=O)CC3)C2)ccc1Nc1ncc2c(n1)N(C(C)C)CC(F)(F)C(=O)N2C]

The compound 155 was synthesized by the method described in the scheme similar to the method described in Example 154. EtOAc

MS(M+H)⁺=830.5, ¹H NMR (400 MHz, DMSO-d₆) δ=11.09 (s, 1H), 9.38 (s, 1H), 8.32 (d, J=8.75 Hz, 1H), 8.23 (s, 1H), 7.89 (s, 1H), 7.72 (t, J=7.75 Hz, 1H), 7.49-7.43 (m, 2H), 7.40-7.32 (m, 2H), 5.10 (dd, J=12.76, 5.38 Hz, 1H), 4.94-4.84 (m, 1H), 4.04 (t, J=13.57 Hz, 2H), 3.94 (s, 3H), 3.33-3.32 (m, 4H), 3.31 (s, 3H), 3.20-3.10 (m, 2H), 3.09-2.99 (m, 3H), 2.99-2.82 (m, 2H), 2.72-2.57 (m, 5H), 2.08-1.96 (m, 2H), 1.85-1.73 (m, 1H), 1.25 (d, J=6.75 Hz, 6H).

### Example 156. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-((1r,4r)-4-((2-(4-(2-(2,6-dioxopiperidin-3-yl)-1,3-dioxoisoindolin-5-yl)piperazin-1-yl)ethyl)(methyl)amino)cyclohexyl)-3-methoxybenzamide (trans) (Compound 156)

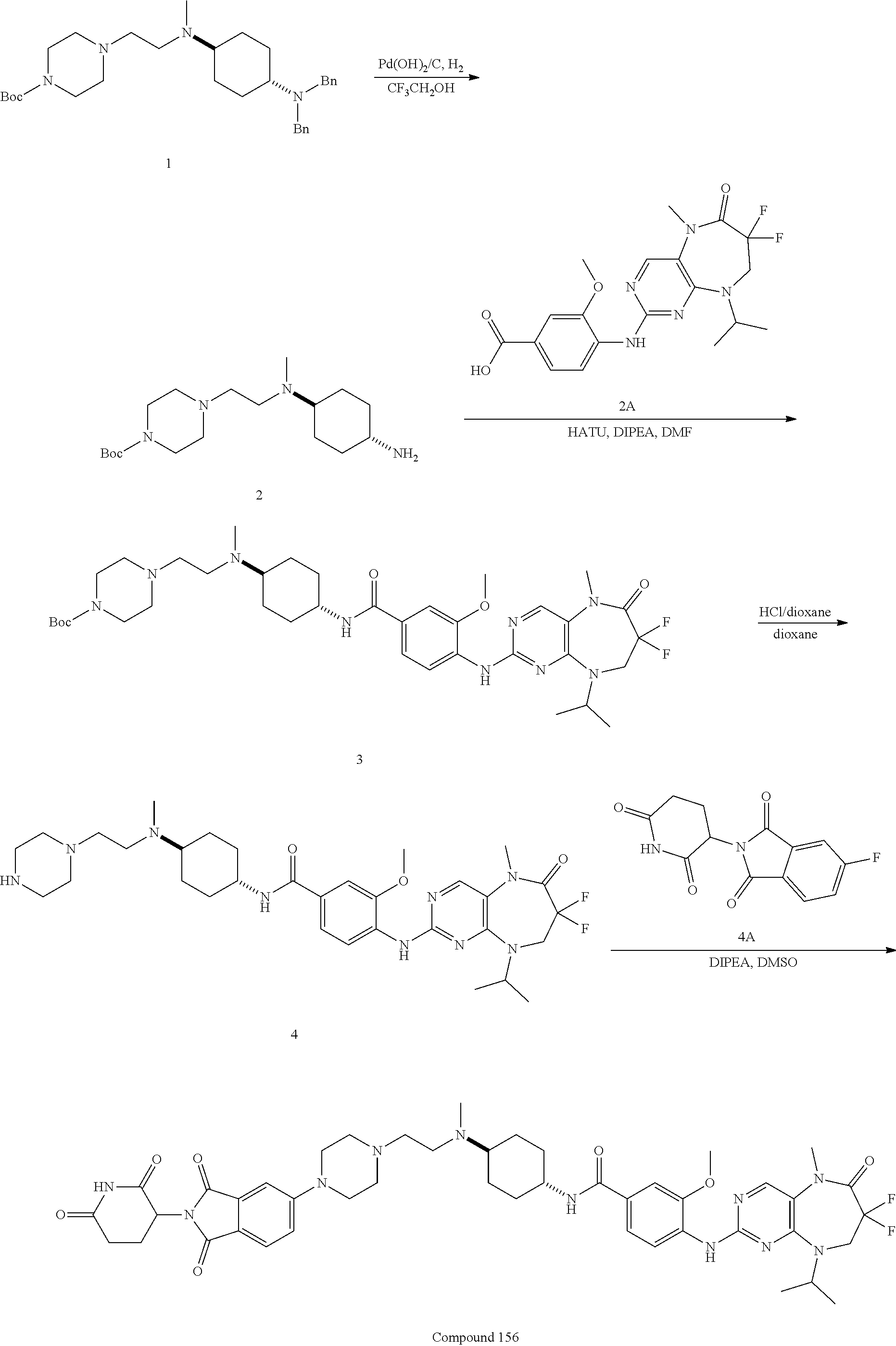

The compound 156 was synthesized by the method described in the scheme similar to the method described in Example 152.

MS(M+H)⁺=900.6, ¹H NMR (400 MHz, DMSO-d₆) δ=11.09 (br s, 1H), 8.34-8.28 (m, 1H), 8.23 (s, 1H), 8.07 (br d, J=7.3 Hz, 1H), 7.88 (s, 1H), 7.69 (d, J=8.8 Hz, 1H), 7.53-7.48 (m, 2H), 7.35 (s, 1H), 7.27 (br d, J=9.0 Hz, 1H), 5.08 (dd, J=5.8, 12.4 Hz, 1H), 4.94-4.84 (m, 1H), 4.05 (br t, J=13.8 Hz, 2H), 3.94 (s, 3H), 3.79-3.70 (m, 1H), 3.47-3.41 (m, 4H), 3.31 (s, 3H), 2.95-2.84 (m, 1H), 2.53-2.43 (m, 9H), 2.23 (s, 3H), 2.07-1.98 (m, 2H), 1.96-1.85 (m, 3H), 1.82-1.75 (m, 2H), 1.41-1.34 (m, 4H), 1.28-1.21 (m, 6H).

### Example 157. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-((4-(2-(2,6-dioxopiperidin-3-yl)-1,3-dioxoisoindolin-4-yl)piperazin-1-yl)methyl)-3,3-difluoropiperidin-1-yl)-3-methoxybenzamide (Compound 157)

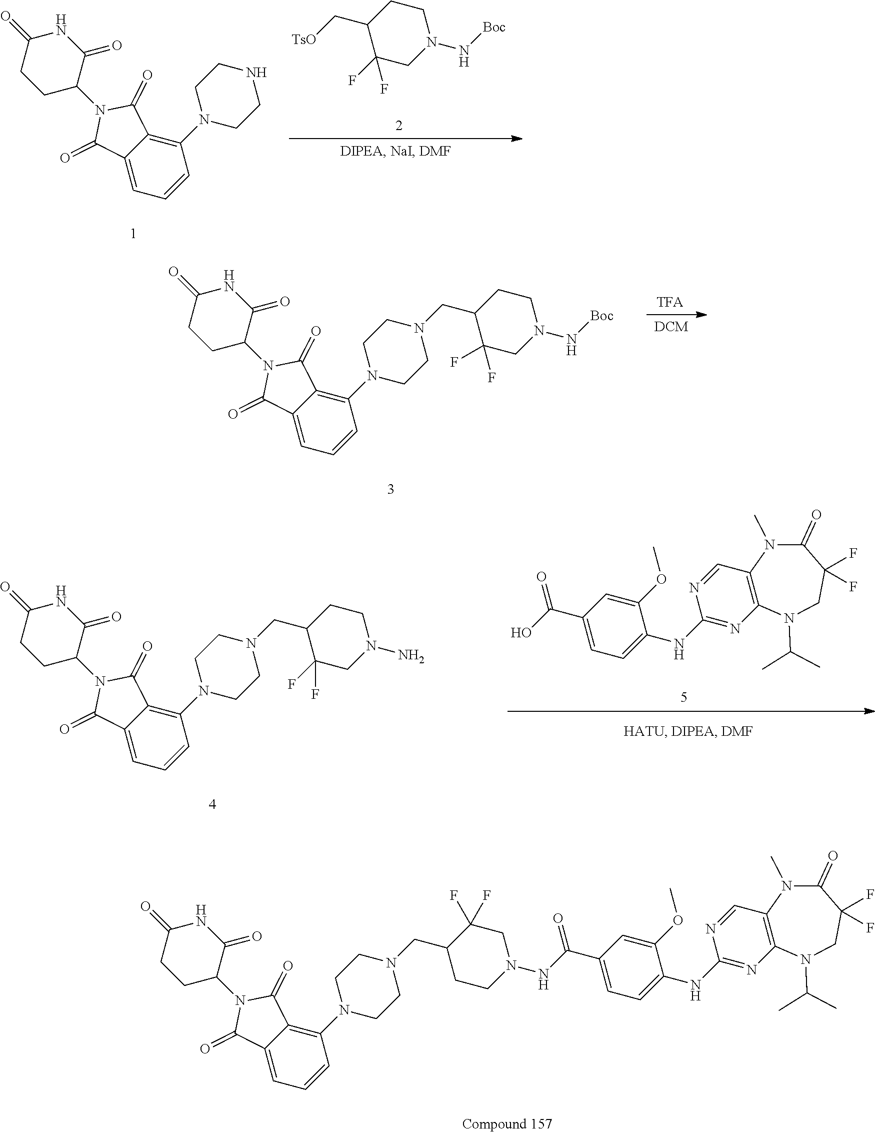 [SMILES: O=C1CCC(N2C(=O)c3cccc(N4CCNCC4)c3C2=O)C(=O)N1 ; Cc1ccc(S(=O)(=O)OCC2CCN(NC(=O)OC(C)(C)C)CC2(F)F)cc1 ; CC(C)(C)OC(=O)NN1CCC(CN2CCN(c3cccc4c3C(=O)N(C3CCC(=O)NC3=O)C4=O)CC2)C(F)(F)C1 ; NN1CCC(CN2CCN(c3cccc4c3C(=O)N(C3CCC(=O)NC3=O)C4=O)CC2)C(F)(F)C1 ; COc1cc(C(=O)O)ccc1Nc1ncc2c(n1)N(C(C)C)CC(F)(F)C(=O)N2C ; COc1cc(C(=O)NN2CCC(CN3CCN(c4cccc5c4C(=O)N(C4CCC(=O)NC4=O)C5=O)CC3)C(F)(F)C2)ccc1Nc1ncc2c(n1)N(C(C)C)CC(F)(F)C(=O)N2C]

### Step 1. Synthesis of tert-butyl (4-((4-(2-(2,6-dioxopiperidin-3-yl)-1,3-dioxoisoindolin-4-yl) piperazin-1-yl)methyl)-3,3-difluoropiperidin-1-yl)carbamate (3)

To a solution of 2-(2,6-dioxopiperidin-3-yl)-4-(piperazin-1-yl)isoindoline-1,3-dione (300 mg, 722.42 μmol, 2HCl) and (1-((tert-butoxycarbonyl)amino)-3,3-difluoropiperidin-4-yl)methyl 4-methylbenzenesulfonate (350 mg, 832.40 μmol) in DMF (6 mL) were added DIPEA (373.46 mg, 2.89 mmol) and NaI (21.66 mg, 144.48 μmol) at 20° C. The resulting mixture was stirred at 60° C. for 14 hrs. LCMS showed the starting material was consumed completely and the desired mass. The mixture was poured into water (10 mL) and extracted with EtOAc (10 mL×3). The combined organic layers were dried over Na₂SO₄ and concentrated to afford the crude product. The crude product was purified by flash silica gel chromatography (4 g silica gel column, EtOAc/petroleum ether=20-60%, 40 mL/min) to afford the crude product which was further purified by prep-TLC (pure EtOAc, Rf=0.4) to afford tert-butyl (4-((4-(2-(2,6-dioxopiperidin-3-yl)-1,3-dioxoisoindolin-4-yl)piperazin-1-yl)methyl)-3,3-difluoropiperidin-1-yl)carbamate (40 mg, 67.73 μmol, 9.37% yield) as yellow solid. MS(M+H)⁺=591.3

### Step 2. Synthesis of 4-(4-((1-amino-3,3-difluoropiperidin-4-yl)methyl)piperazin-1-yl)-2-(2,6-dioxopiperidin-3-yl)isoindoline-1,3-dione (4)

To a solution of tert-butyl (4-((4-(2-(2,6-dioxopiperidin-3-yl)-1,3-dioxoisoindolin-4-yl) piperazin-1-yl)methyl)-3,3-difluoropiperidin-1-yl)carbamate (30 mg) in DCM (5 mL) was added TFA (3.08 g, 2 mL) at 20° C. The resulting solution was stirred at 20° C. for 30 mins. LCMS showed a main peak with desired mass. The reaction solution was concentrated to afford 4-(4-((1-amino-3,3-difluoropiperidin-4-yl)methyl)piperazin-1-yl)-2-(2,6-dioxopiperidin-3-yl)isoindoline-1,3-dione (15 mg, crude, 2TFA) as yellow oil. MS(M+H)⁺=491.2

### Step 3. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-((4-(2-(2,6-dioxopiperidin-3-yl)-1,3-dioxoisoindolin-4-yl)piperazin-1-yl)methyl)-3,3-difluoropiperidin-1-yl)-3-methoxybenzamide (Compound 157)

To a solution of 4-((7,7-difluoro-9-isopropyl-5-methyl-6-oxo-6,7,8,9-tetrahydro-5H-pyrimido[4,5-b][1, 4]diazepin-2-yl)amino)-3-methoxybenzoic acid (21.11 mg, 50.10 μmol), DIPEA (35.97 mg, 278.34 μmol) and HATU (21.17 mg, 55.67 μmol) in DMF (1 mL) was added 4-(4-((1-amino-3,3-difluoropiperidin-4-yl)methyl)piperazin-1-yl)-2-(2,6-dioxopiperidin-3-yl)isoindoline-1,3-dione (40 mg, crude, 2TFA) at 20° C. The resulting mixture was stirred at 20° C. for 1 h. LCMS showed the starting material was consumed completely and the desired mass. The reaction solution was poured into water (10 mL) and extracted with EtOAc (5 mL×4). The combined organic layers were dried over Na₂SO 4 and concentrated to afford the crude product. The crude product was further purified by prep-HPLC (column: Waters Xbridge 150*25 mm*5 μm; mobile phase: [water (NH₄HCO₃)-ACN]; B %: 40%-70%, 9 min) and lyophilized to afford 4-((7,7-difluoro-9-isopropyl-5-methyl-6-oxo-6,7,8,9-tetrahydro-5H-pyrimido[4,5-b][1, 4]diazepin-2-yl)amino)-N-(4-((4-(2-(2,6-dioxopiperidin-3-yl)-1,3-dioxoisoindolin-4-yl)piperazin-1-yl)methyl)-3,3-difluoropiperidin-1-yl)-3-methoxybenzamide (7.1 mg, 7.36 μmol, 13.23% yield, 92.7% purity) as yellow solid. MS(M+H)⁺=893.9

¹H NMR (400 MHz, CDCl₃) δ=8.48 (d, J=8.6 Hz, 1H), 8.04 (s, 1H), 8.01 (s, 1H), 7.74 (s, 1H), 7.69 (br s, 1H), 7.64-7.59 (m, 1H), 7.42 (d, J=7.0 Hz, 1H), 7.38 (s, 1H), 7.26-7.21 (m, 1H), 7.18 (d, J=8.4 Hz, 1H), 5.03-4.93 (m, 2H), 3.98 (s, 3H), 3.88 (t, J=13.0 Hz, 2H), 3.69-3.55 (m, 1H), 3.52-3.33 (m, 9H), 3.30-3.26 (m, 1H), 2.95-2.84 (m, 2H), 2.84-2.72 (m, 4H), 2.70-2.59 (m, 2H), 2.57-2.47 (m, 1H), 2.19-2.02 (m, 3H), 1.75-1.68 (m, 1H), 1.33 (d, J=6.7 Hz, 6H).

### Example 158. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-((4-(2-(2,6-dioxopiperidin-3-yl)-1-oxoisoindolin-4-yl)piperazin-1-yl)methyl)-3,3-difluoropiperidin-1-yl)-3-methoxybenzamide (Compound 158)

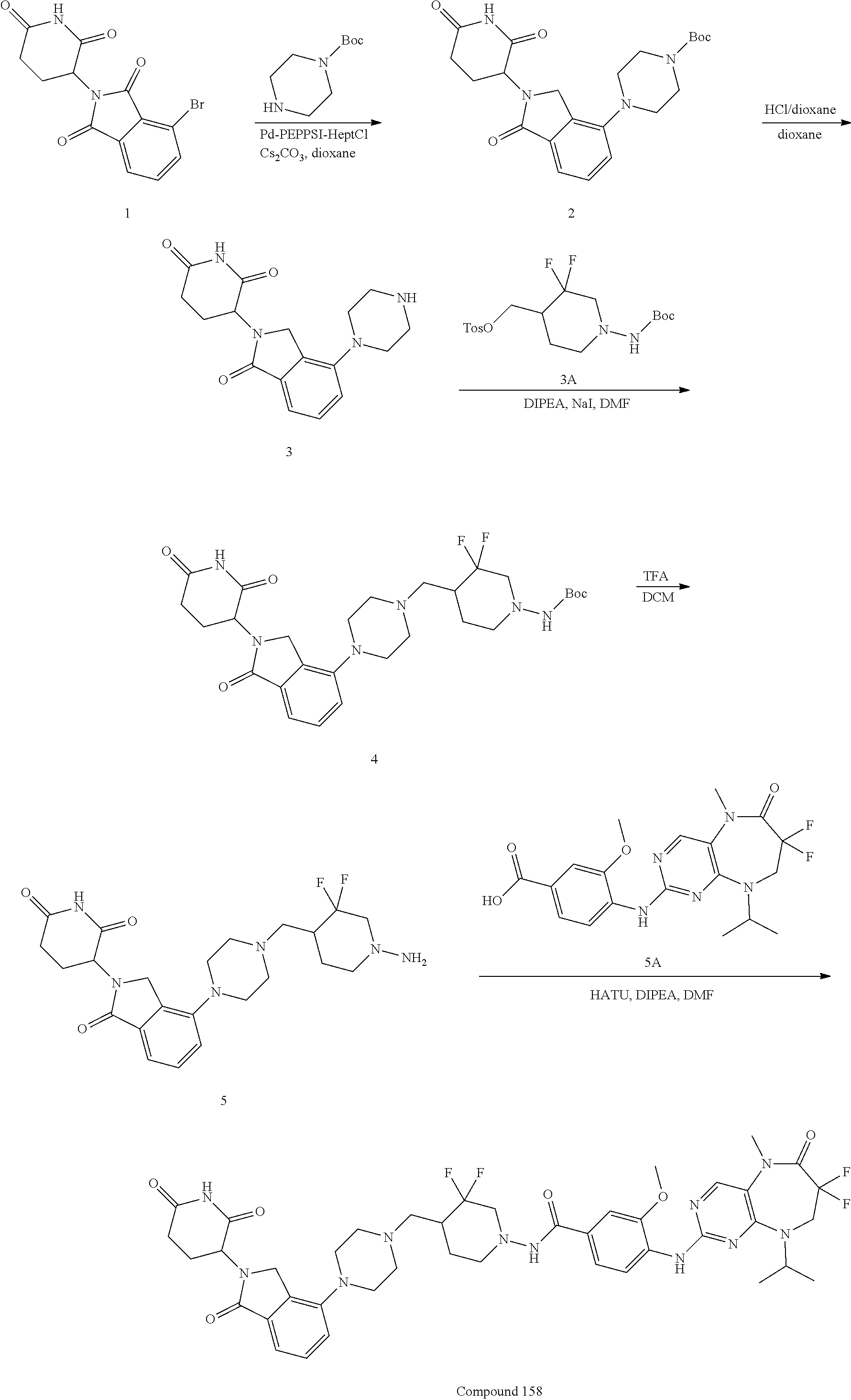

The compound 158 was synthesized by the method described in the scheme similar to the method described in Example 157.

MS(M+H)⁺=880.4. ¹H NMR (400 MHz, CDCl₃) δ=8.48 (d, J=8.44 Hz, 1H), 8.08-8.02 (m, 2H), 7.77-7.69 (m, 2H), 7.57 (d, J=7.21 Hz, 1H), 7.45 (t, J=7.70 Hz, 1H), 7.39 (s, 1H), 7.25 (s, 1H), 7.16 (d, J=7.95 Hz, 1H), 5.26 (dd, J=13.14, 5.07 Hz, 1H), 5.02-4.93 (m, 1H), 4.51-4.27 (m, 2H), 3.98 (s, 3H), 3.88 (t, J=13.08 Hz, 2H), 3.73-3.56 (m, 1H), 3.53-3.36 (m, 5H), 3.33-3.25 (m, 1H), 3.18-3.01 (m, 4H), 2.99-2.86 (m, 2H), 2.82-2.67 (m, 3H), 2.61-2.52 (m, 2H), 2.50-2.34 (m, 2H), 2.29-2.19 (m, 1H), 2.16-2.02 (m, 2H), 1.74-1.67 (m, 1H), 1.33 (d, J=6.85 Hz, 6H).

### Example 159. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(3-(4-(2-(2,6-dioxopiperidin-3-yl)-1,3-dioxoisoindolin-5-yl)piperazin-1-yl)azetidin-1-yl)-3-methoxybenzamide (Compound 159)

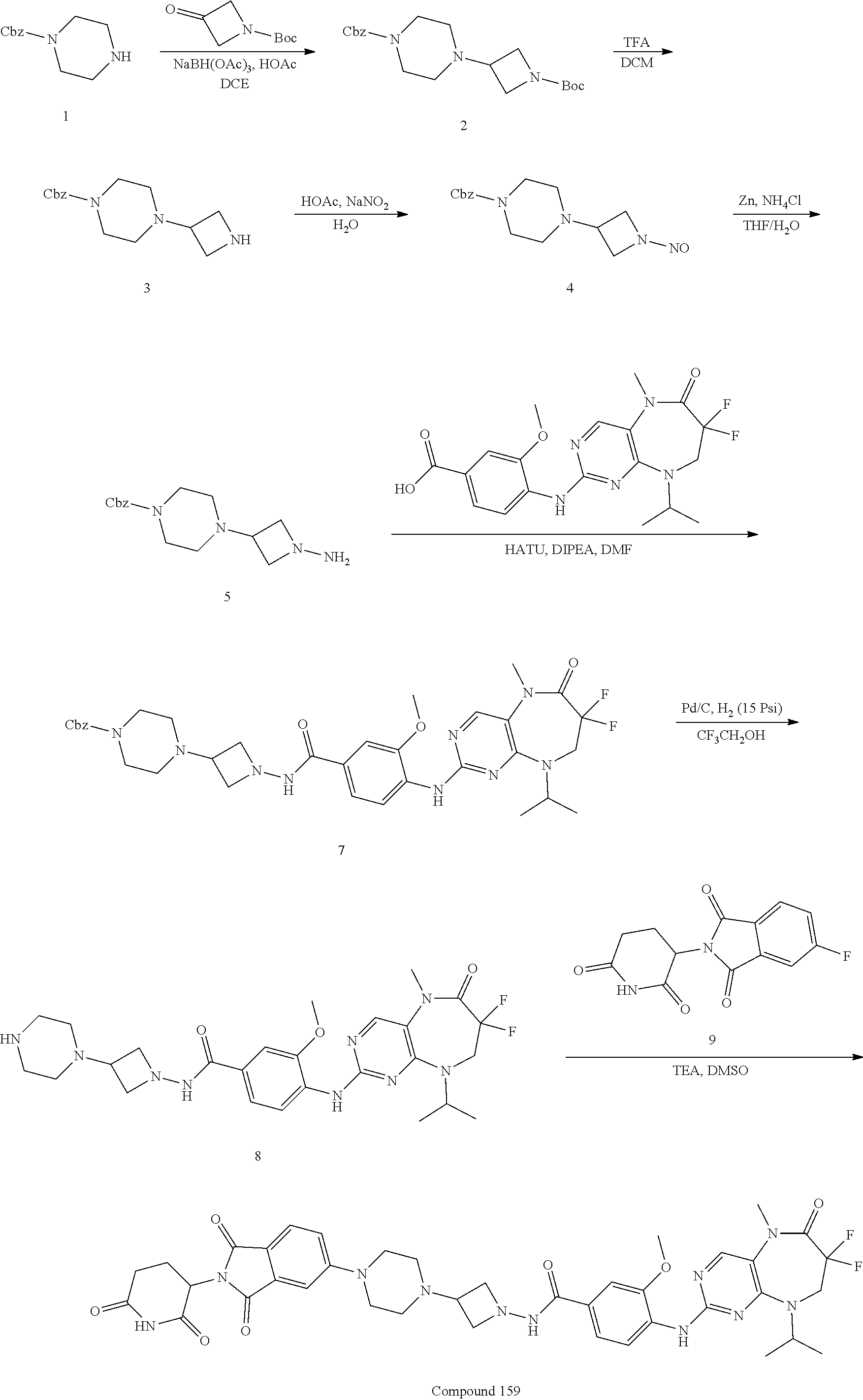

### Step 1. Synthesis of benzyl 4-(1-(tert-butoxycarbonyl)azetidin-3-yl)piperazine-1-carboxylate (2)

To a solution of benzyl piperazine-1-carboxylate (8 g, 36.32 mmol, 7.02 mL), tert-butyl 3-oxoazetidine-1-carboxylate (6.22 g, 36.32 mmol) in DCE (80 mL) were added NaBH(OAc)₃ (15.40 g, 72.64 mmol) and HOAc (1.09 g, 18.16 mmol, 1.04 mL). The mixture was stirred at 25° C. for 2 h. TLC (petroleum ether:EtOAc=1:2) indicated two new spots were formed. Saturated sodium bicarbonate solution (200 mL) was added to the mixture slowly at 0° C. Then the mixture was extracted with DCM (100 mL×3). The combined organic layers were washed with brine (150 mL), dried over anhydrous sodium sulfate, filtered and concentrated under reduced pressure to afford benzyl 4-(1-(tert-butoxycarbonyl)azetidin-3-yl)piperazine-1-carboxylate (14 g, crude) as yellow oil. MS(M+H)⁺=376.3

### Step 2. Synthesis of benzyl 4-(azetidin-3-yl)piperazine-1-carboxylate (3)

To a solution of benzyl 4-(1-(tert-butoxycarbonyl)azetidin-3-yl)piperazine-1-carboxylate (14 g, 37.29 mmol) in DCM (50 mL) was added TFA (15.40 g, 135.06 mmol, 10 mL). The mixture was stirred at 25° C. for 14 h. LCMS showed ˜60% of desired mass was detected. The mixture was concentrated under reduced pressure to afford benzyl 4-(azetidin-3-yl)piperazine-1-carboxylate (25 g, crude, TFA salt) as yellow oil. MS(M+H)⁺=275.9

### Step 3. Synthesis of benzyl 4-(1-nitrosoazetidin-3-yl)piperazine-1-carboxylate (4)

To a solution of benzyl 4-(azetidin-3-yl)piperazine-1-carboxylate (25 g, crude, TFA) in H₂O (40 mL) were added a solution of NaNO₂ (6.64 g, 96.31 mmol) in H₂O (40 mL) and HOAc (5.78 g, 96.31 mmol, 5.51 mL). The mixture was stirred at 25° C. for 2 h. LCMS showed a peak (˜70%) with desired mass. The pH of the reaction mixture was adjusted to around 8 by saturated sodium bicarbonate solution and extracted with EtOAc (100 mL×3). The combined organic layers were washed with saturated brine (150 mL), dried over anhydrous sodium sulfate, filtered and concentrated under reduced pressure to give benzyl 4-(1-nitrosoazetidin-3-yl)piperazine-1-carboxylate (12 g, crude) as yellow oil. MS(M+H)⁺=305.2

### Step 4. Synthesis of benzyl 4-(1-aminoazetidin-3-yl)piperazine-1-carboxylate (5)

To a solution of benzyl 4-(1-nitrosoazetidin-3-yl)piperazine-1-carboxylate (12 g, crude) in THF (30 mL) were added a solution of NH₄Cl (8.44 g, 157.72 mmol) in H₂O (30 mL) and Zn (5.47 g, 83.65 mmol) at 0° C. The mixture was stirred at 25° C. for 2 h. LCMS showed a peak (˜55%) with desired mass. The mixture was filtered. The filtrate was concentrated under reduced pressure. The residue was purified by reverse phase HPLC (Column: 330 g Flash Column Welch Ultimate XB_C18 20-40 μm; 120 A, Mobile phase: [water(NH₃H₂O)-ACN]; B %: 0%-60%, 100 mL/min) followed by lyophilization to afford benzyl 4-(1-aminoazetidin-3-yl)piperazine-1-carboxylate (7.3 g, 23.13 mmol, 58.66% yield, 92% purity) as yellow oil. MS(M+H)⁺=291.1

### Step 5. Synthesis of benzyl 4-(1-(4-((7,7-difluoro-9-isopropyl-5-methyl-6-oxo-6,7,8,9-tetrahydro-5H-pyrimido[4,5-b][1,4]diazepin-2-yl)amino)-3-methoxybenzamido)azetidin-3-yl)piperazine-1-carboxylate (7)

To a solution of 4-((7,7-difluoro-9-isopropyl-5-methyl-6-oxo-6,7,8,9-tetrahydro-5H-pyrimido[4,5-b][1, 4]diazepin-2-yl)amino)-3-methoxybenzoic acid (1.18 g, 2.79 mmol) in DMF (10 mL) were added HATU (1.77 g, 4.65 mmol) and DIPEA (1.20 g, 9.30 mmol, 1.62 mL). Then benzyl 4-(1-aminoazetidin-3-yl)piperazine-1-carboxylate (0.9 g, 3.10 mmol) was added to the mixture after 0.5 h. The mixture was stirred at 25° C. for 12 h. LCMS showed a peak (˜44%) with desired mass. The reaction mixture was diluted with water (50 mL) and extracted with EtOAc (100 mL×3). The combined organic layers were washed with saturated brine (150 mL), dried over anhydrous sodium sulfate, filtered and concentrated under reduced pressure. The residue was purified by flash silica gel chromatography (ISCO®; 20 g Sepa Flash® Silica Flash Column, Eluent of 0˜60% petroleum ether:EtOAc/ethanol (1:1) gradient @60 mL/min) to afford benzyl 4-(1-(4-((7,7-difluoro-9-isopropyl-5-methyl-6-oxo-6,7,8,9-tetrahydro-5H-pyrimido[4,5-b][1,4]diazepin-2-yl)amino)-3-methoxybenzamido)azetidin-3-yl)piperazine-1-carboxylate (0.96 g, 1.35 mmol, 43.53% yield, 97.5% purity) as yellow oil. MS(M+H)⁺=694.4

### Step 6. Synthesis of 4-((7,7-difluoro-9-isopropyl-5-methyl-6-oxo-6,7,8,9-tetrahydro-5H-pyrimido[4,5-b][1,4]diazepin-2-yl)amino)-3-methoxy-N-(3-(piperazin-1-yl)azetidin-1-yl)benzamide (8)

To a solution of benzyl 4-(1-(4-((7,7-difluoro-9-isopropyl-5-methyl-6-oxo-6,7,8,9-tetrahydro-5H-pyrimido[4,5-b][1,4]diazepin-2-yl)amino)-3-methoxybenzamido)azetidin-3-yl)piperazine-1-carboxylate (0.96 g, 1.38 mmol) in CF₃CH₂OH (30 mL) was added Pd/C (0.1 g, 691.90 μmol, 10% purity) under N₂ atmosphere. The mixture was degassed and purged with H₂ for 3 times and the suspension was stirred at 25° C. for 12 h under H₂ (15 psi). LCMS showed a major peak (˜90%) with desired mass. The mixture was filtered. The filtrate was concentrated under reduced pressure to give 4-((7,7-difluoro-9-isopropyl-5-methyl-6-oxo-6,7,8,9-tetrahydro-5H-pyrimido[4,5-b][1, 4]diazepin-2-yl)amino)-3-methoxy-N-(3-(piperazin-1-yl)azetidin-1-yl)benzamide (720 mg, crude) as a yellow solid. MS(M+H)⁺=560.4

### Step 7. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(3-(4-(2-(2,6-dioxopiperidin-3-yl)-1,3-dioxoisoindolin-5-yl)piperazin-1-yl)azetidin-1-yl)-3-methoxybenzamide (Compound 159)

To a solution of 4-((7,7-difluoro-9-isopropyl-5-methyl-6-oxo-6,7,8,9-tetrahydro-5H-pyrimido[4,5-b][1, 4]diazepin-2-yl)amino)-3-methoxy-N-(3-(piperazin-1-yl)azetidin-1-yl)benzamide (100 mg, crude) in DMSO (2 mL) were added TEA (54.25 mg, 536.09 μmol, 74.62 μL) and 2-(2,6-dioxopiperidin-3-yl)-5-fluoroisoindoline-1,3-dione (49.36 mg, 178.70 μmol) at 25° C. The mixture was stirred at 100° C. for 2 h. LCMS showed a peak (˜17%) with desired mass. The reaction mixture was diluted with water (20 mL) and extracted with EtOAc (20 mL×3). The combined organic layers were washed with brine (50 mL), dried over anhydrous sodium sulfate, filtered and concentrated under reduced pressure. The residue was purified by prep-HPLC (column: Phenomenex luna C18 150×25 mm×10 μm; mobile phase: [water(FA)-ACN]; B %: 11%-41%, 10 min) followed by prep-HPLC (column: Waters Xbridge 150×25 mm×5 μm; mobile phase: [water(NH₄HCO₃)-ACN]; B %: 33%-63%, 8 min), the eluent was lyophilized to afford 4-((7,7-difluoro-9-isopropyl-5-methyl-6-oxo-6,7,8,9-tetrahydro-5H-pyrimido[4,5-b][1, 4]diazepin-2-yl)amino)-N-(3-(4-(2-(2,6-dioxopiperidin-3-yl)-1,3-dioxoisoindolin-5-yl) piperazin-1-yl)azetidin-1-yl)-3-methoxybenzamide (11.6 mg, 13.85 μmol, 7.75% yield, 97.4% purity) as a yellow solid. MS(M+H)⁺=816.4.

¹H NMR (400 MHz, DMSO-d₆) δ=11.08 (s, 1H), 9.53 (s, 1H), 8.34-8.27 (m, 1H), 8.22 (s, 1H), 7.88 (s, 1H), 7.69 (d, J=8.6 Hz, 1H), 7.49-7.42 (m, 2H), 7.37 (s, 1H), 7.29 (d, J=8.6 Hz, 1H), 5.08 (dd, J=5.3, 12.9 Hz, 1H), 4.92-4.82 (m, 1H), 4.03 (t, J=13.5 Hz, 2H), 3.93 (s, 3H), 3.78 (t, J=6.4 Hz, 2H), 3.67-3.63 (m, 2H), 3.45-3.50 (m, 4H), 3.29 (s, 3H), 2.97-2.83 (m, 2H), 2.61-2.56 (m, 2H), 2.47-2.42 (m, 4H), 2.07-1.98 (m, 1H), 1.24 (d, J=6.7 Hz, 6H).

### Example 160. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(3-(4-(2-(2,6-dioxopiperidin-3-yl)-1,3-dioxoisoindolin-4-yl)piperazin-1-yl)azetidin-1-yl)-3-methoxybenzamide (Compound 160)

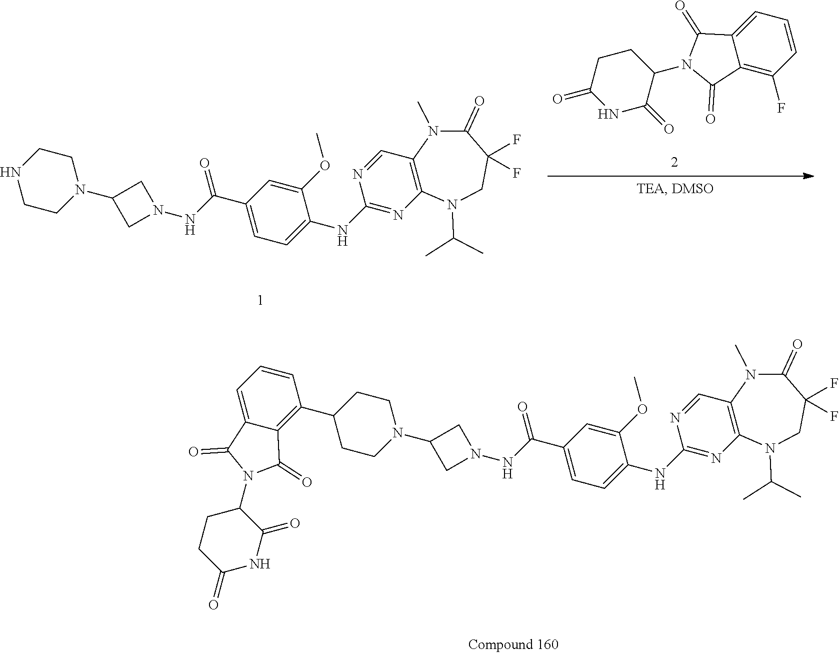 [SMILES: COc1cc(C(=O)NN2CC(N3CCNCC3)C2)ccc1Nc1ncc2c(n1)N(C(C)C)CC(F)(F)C(=O)N2C ; O=C1CCC(N2C(=O)c3cccc(F)c3C2=O)C(=O)N1 ; COc1cc(C(=O)NN2CC(N3CCC(c4cccc5c4C(=O)N(C4CCC(=O)NC4=O)C5=O)CC3)C2)ccc1Nc1ncc2c(n1)N(C(C)C)CC(F)(F)C(=O)N2C]

The compound 160 was synthesized by the method described in the scheme similar to the method described in Example 159.

MS(M+H)⁺=816.4, ¹H NMR (400 MHz, DMSO-d₆) δ=11.09 (s, 1H), 9.52 (s, 1H), 8.34-8.28 (m, 1H), 8.22 (s, 1H), 7.88 (s, 1H), 7.76-7.67 (m, 1H), 7.49-7.41 (m, 2H), 7.36 (dd, J=5.0, 7.5 Hz, 2H), 5.09 (dd, J=5.3, 12.7 Hz, 1H), 4.90-4.84 (m, 1H), 4.04 (t, J=13.6 Hz, 2H), 3.93 (s, 3H), 3.77 (t, J=6.5 Hz, 2H), 3.68-3.65 (m, 2H), 3.36-3.33 (m, 4H), 3.30 (s, 3H), 3.01-2.82 (m, 2H), 2.64-2.54 (m, 6H), 2.09-1.98 (m, 1H), 1.24 (d, J=6.7 Hz, 6H).

### Example 161. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(3-((4-(2-(2,6-dioxopiperidin-3-yl)-1,3-dioxoisoindolin-4-yl)piperazin-1-yl)methyl)azetidin-1-yl)-3-methoxybenzamide (Compound 161)

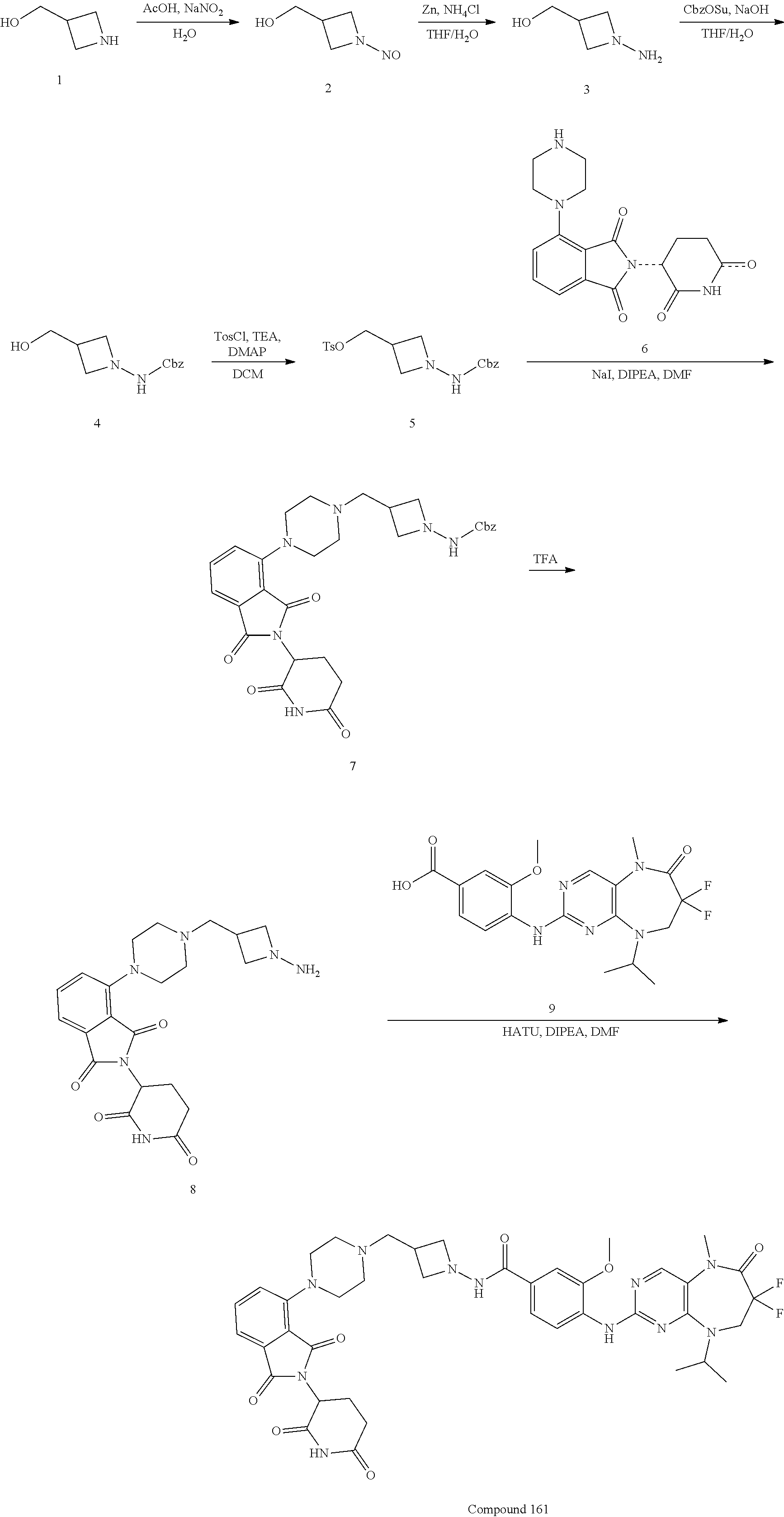

### Step 1. Synthesis of (1-nitrosoazetidin-3-yl)methanol (2)

To a solution of azetidin-3-ylmethanol (9.5 g, 76.87 mmol, HCl salt) in H₂O (40 mL) were added a solution of NaNO₂ (7.96 g, 115.31 mmol) in H₂O (40 mL) and HOAc (6.92 g, 115.31 mmol, 6.59 mL) at 0° C. The mixture was stirred at 25° C. for 5 h. TLC (petroleum ether:EtOAc=1:2) indicated azetidin-3-ylmethanol was consumed completely and one new spot was formed. The pH of the reaction mixture was adjusted to around 7 with saturated sodium bicarbonate solution. Then the mixture was extracted with EtOAc/ethanol (v/v=10/1) (100 mL×15). The combined organic layers were concentrated under reduced pressure to afford (1-nitrosoazetidin-3-yl)methanol (9 g, crude) as yellow oil. MS(M+H)⁺=117.2

### Step 2. Synthesis of (1-aminoazetidin-3-yl)methanol (3)

To a solution of (1-nitrosoazetidin-3-yl)methanol (9 g, crude) in THF (30 mL) were added Zn (18.98 g, 290.26 mmol) and a solution of NH₄Cl (16.58 g, 310.03 mmol) in H₂O (30 mL) at 0° C. The mixture was stirred at 25° C. for 2 h. TLC (petroleum ether:EtOAc=10:1) indicated (1-nitrosoazetidin-3-yl)methanol was consumed completely. The mixture was filtered to remove insoluble solid. The filtrate was concentrated under reduced pressure to afford (1-aminoazetidin-3-yl)methanol (19 g, crude) as yellow oil. MS(M+H)⁺=103.1

### Step 3. Synthesis of benzyl (3-(hydroxymethyl)azetidin-1-yl)carbamate (4)

To a solution of (1-aminoazetidin-3-yl)methanol (2 g, 19.58 mmol) in THF (10 mL) were added benzyl (2,5-dioxopyrrolidin-1-yl) carbonate (4.88 g, 19.58 mmol) and a solution of NaOH (3.92 g, 97.91 mmol) in H₂O (10 mL). The mixture was stirred at 25° C. for 2 h. LCMS showed a peak (30%) with desired mass. The reaction mixture was diluted with water (50 mL) and extracted with EtOAc (50 mL×3). The combined organic layers were washed with brine (50 mL), dried over anhydrous sodium sulfate, filtered and concentrated under reduced pressure to afford benzyl (3-(hydroxymethyl)azetidin-1-yl)carbamate (3.2 g, 9.75 mmol, 49.80% yield, 72% purity) as yellow oil. MS (M+H)⁺=237.2

### Step 4. Synthesis of (1-(((benzyloxy)carbonyl)amino)azetidin-3-yl)methyl 4-methylbenzenesulfonate (5)

To a solution of benzyl (3-(hydroxymethyl)azetidin-1-yl)carbamate (3 g, 12.70 mmol) in DCM (30 mL) were added TosCl (2.42 g, 12.70 mmol), TEA (2.57 g, 25.40 mmol, 3.53 mL) and DMAP (775.62 mg, 6.35 mmol). The mixture was stirred at 20° C. for 2 h. LCMS showed a peak (50%) with desired mass. The reaction mixture was diluted with water (50 mL) and extracted with DCM (50 mL×3). The combined organic layers were washed with brine (50 mL), dried over anhydrous sodium sulfate, filtered and concentrated under reduced pressure. The residue was purified by flash silica gel chromatography (ISCO®; 40 g SepaFlash® Silica Flash Column, Eluent of 0˜50% petroleum ether:EtOAc gradient @80 mL/min) to afford (1-(((benzyloxy)carbonyl)amino)azetidin-3-yl)methyl 4-methylbenzenesulfonate (1.4 g, 3.44 mmol, 27.11% yield, 96% purity) as yellow oil. MS(M+H)⁺=391.1

### Step 5. Synthesis of benzyl (3-((4-(2-(2,6-dioxopiperidin-3-yl)-1,3-dioxoisoindolin-4-yl)piperazin-1-yl)methyl) azetidin-1-yl)carbamate (7)

To a solution of 2-(2,6-dioxopiperidin-3-yl)-4-(piperazin-1-yl)isoindoline-1,3-dione (776.14 mg, 2.05 mmol, HCl salt) in DMF (8 mL) were added (1-(((benzyloxy)carbonyl)amino)azetidin-3-yl)methyl 4-methylbenzenesulfonate (0.8 g, 2.05 mmo), DIPEA (794.42 mg, 6.15 mmol, 1.07 mL) and NaI (153.56 mg, 1.02 mmol). The mixture was stirred at 80° C. for 12 h. LCMS showed a peak (10%) with desired mass. The mixture was diluted with water (50 mL) and extracted with EtOAc (50 mL×3). The combined organic layers were washed with brine (50 mL), dried over anhydrous sodium sulfate, filtered and concentrated under reduced pressure. The residue was purified by flash silica gel chromatography (ISCO®; 20 g SepaFlash® Silica Flash Column, Eluent of 0˜60% petroleum ether:EtOAc/ethanol (v/v=1/1) gradient @80 mL/min) to afford benzyl (3-((4-(2-(2,6-dioxopiperidin-3-yl)-1,3-dioxoisoindolin-4-yl)piperazin-1-yl)methyl)azetidin-1-yl)carbamate (320 mg, 508.03 μmol, 24.80% yield, 89% purity) as a yellow solid. MS(M+H)⁺=561.1

### Step 6. Synthesis of 4-(4-((1-aminoazetidin-3-yl)methyl)piperazin-1-yl)-2-(2,6-dioxopiperidin-3-yl)isoindoline-1,3-dione (8)

A solution of benzyl (3-((4-(2-(2,6-dioxopiperidin-3-yl)-1,3-dioxoisoindolin-4-yl)piperazin-1-yl)methyl)azetidin-1-yl)carbamate (200 mg, 356.76 μmol) in TFA (5 mL) was stirred at 40° C. for 4 h. LCMS showed a peak (15%) with desired mass. The mixture was concentrated under reduced pressure to afford 4-(4-((1-aminoazetidin-3-yl)methyl)piperazin-1-yl)-2-(2,6-dioxopiperidin-3-yl)isoindoline-1,3-dione (0.2 g, crude, TFA salt) as yellow oil. MS(M+H)⁺=427.1

### Step 7. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(3-((4-(2-(2,6-dioxopiperidin-3-yl)-1,3-dioxoisoindolin-4-yl)piperazin-1-yl)methyl)azetidin-1-yl)-3-methoxybenzamide (Compound 161)

To a solution of 4-((7,7-difluoro-9-isopropyl-5-methyl-6-oxo-6,7,8,9-tetrahydro-5H-pyrimido[4,5-b][1, 4]diazepin-2-yl)amino)-3-methoxybenzoic acid (60 mg, 142.38 μmol) in DMF (2 mL) were added HATU (81.21 mg, 213.58 μmol) and DIPEA (92.01 mg, 711.92 μmol, 124.00 μL), after stirring for 0.5 h. Then 4-(4-((1-aminoazetidin-3-yl)methyl)piperazin-1-yl)-2-(2,6-dioxopiperidin-3-yl)isoindoline-1,3-dione (192.39 mg, crude, TFA salt) was added and the resulting mixture was stirred at 25° C. for 1 h. LCMS showed a peak (50%) with desired mass. The reaction mixture was diluted with water (50 mL) and extracted with EtOAc (50 mL×3). The combined organic layers were washed with brine (50 mL), dried over anhydrous sodium sulfate, filtered and concentrated under reduced pressure. The residue was purified by flash silica gel chromatography (ISCO®; 20 g SepaFlash® Silica Flash Column, Eluent of 0˜100% petroleum ether/methanol gradient @80 mL/min) followed by prep-HPLC (column: Waters Xbridge 150×50 mm×10 μm; mobile phase: [water(NH₄HCO₃)-ACN]; B %: 27%-57%, 10 min) the eluent was lyophilized to afford 4-((7,7-difluoro-9-isopropyl-5-methyl-6-oxo-6,7,8,9-tetrahydro-5H-pyrimido[4,5-b][1, 4]diazepin-2-yl)amino)-N-(3-((4-(2-(2,6-dioxopiperidin-3-yl)-1,3-dioxoisoindolin-4-yl)piperazin-1-yl)methyl)azetidin-1-yl)-3-methoxybenzamide (47.24 mg, 53.57 μmol, 37.62% yield, 94.1% purity) as a yellow solid. MS(M+H)⁺=830.4.

¹H NMR (400 MHz, DMSO-d₆) δ=11.07 (s, 1H), 9.46 (s, 1H), 8.32-8.27 (m, 1H), 8.22 (s, 1H), 7.88 (s, 1H), 7.70 (dd, J=7.3, 8.3 Hz, 1H), 7.45-7.39 (m, 2H), 7.35 (dd, J=7.9, 9.8 Hz, 2H), 5.09 (dd, J=5.4, 12.8 Hz, 1H), 4.90-4.84 (m, 1H), 4.03 (t, J=13.5 Hz, 2H), 3.93 (s, 3H), 3.80 (t, J=6.6 Hz, 2H), 3.55-3.47 (m, 2H), 3.32-3.27 (m, 7H), 2.95-2.83 (m, 1H), 2.64-2.54 (m, 9H), 2.07-1.98 (m, 1H), 1.24 (d, J=6.6 Hz, 6H).

### Example 162. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(1-((1-(2-(2,6-dioxopiperidin-3-yl)-6-fluoro-1,3-dioxoisoindolin-5-yl)azetidin-3-yl)methyl)piperidin-4-yl)-3-methoxybenzamide (Compound 162)

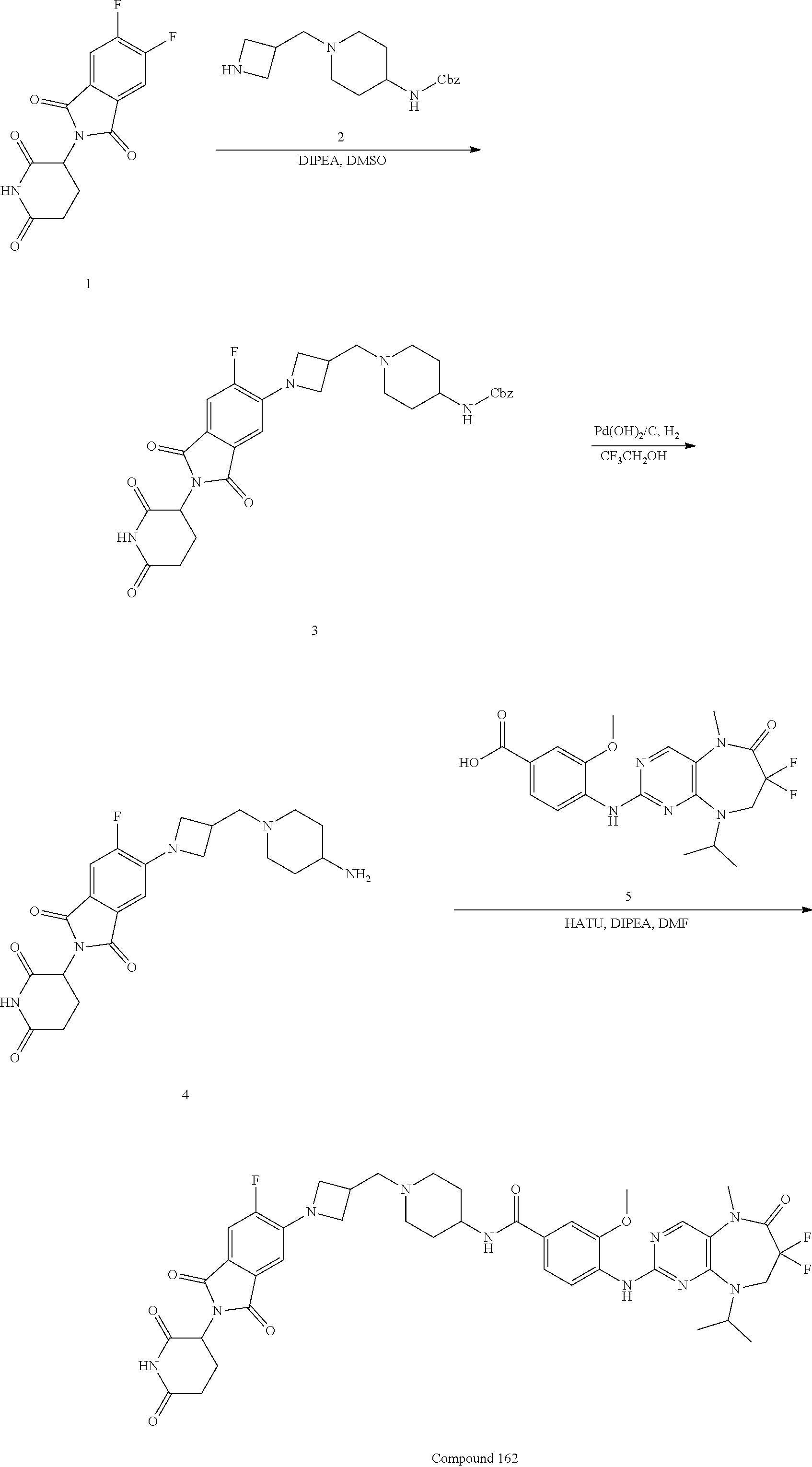

### Step 1. Synthesis of benzyl (1-((1-(2-(2,6-dioxopiperidin-3-yl)-6-fluoro-1,3-dioxoisoindolin-5-yl)azetidin-3-yl) methyl)piperidin-4-yl)carbamate (3)

To a solution of 2-(2,6-dioxopiperidin-3-yl)-5,6-difluoroisoindoline-1,3-dione (500 mg, 1.70 mmol) and benzyl (1-(azetidin-3-ylmethyl)piperidin-4-yl)carbamate (1.14 g, 2.72 mmol, TFA salt) in DMSO (10 mL) was added DIPEA (1.10 g, 8.50 mmol, 1.48 mL), the mixture was stirred at 100° C. for 16 hours. LCMS showed a peak (14%) with desired mass. The reaction mixture was diluted with H₂O (100 mL) and extracted with EtOAc (60 mL×3). The combined organic layers were washed with brine (100 mL×5), dried over Na₂SO₄, filtered and concentrated in vacuum. The residue was purified by flash silica gel chromatography (12 g SepaFlash® Silica Flash Column, Eluent of 0˜5% Methanol/EtOAc gradient @100 mL/min) followed by prep-HPLC (column: Phenomenex C18 75*30 mm*3 μm; mobile phase: [water(FA)-ACN]; B %: 8%-38%, 7 min), the eluent was freeze-dried to afford benzyl (1-((1-(2-(2,6-dioxopiperidin-3-yl)-6-fluoro-1,3-dioxoisoindolin-5-yl)azetidin-3-yl)methyl)piperidin-4-yl)carbamate (350 mg, 605.95 μmol, 35.66% yield) as a yellow solid. MS(M+H)⁺=578.1.

¹H NMR (400 MHz, DMSO-d₆) δ=11.08 (s, 1H), 7.58 (d, J=11.3 Hz, 1H), 7.45-7.27 (m, 5H), 7.23 (br d, J=7.8 Hz, 1H), 6.89 (d, J=7.8 Hz, 1H), 5.13-4.90 (m, 3H), 4.24 (br t, J=7.3 Hz, 2H), 3.80 (br t, J=6.2 Hz, 2H), 3.50-3.37 (m, 1H), 3.01-2.82 (m, 2H), 2.77 (br d, J=11.1 Hz, 2H), 2.64-2.53 (m, 4H), 2.08-1.88 (m, 3H), 1.71 (br d, J=10.5 Hz, 2H), 1.49-1.28 (m, 2H).

### Step 2. Synthesis of 5-(3-((4-aminopiperidin-1-yl)methyl)azetidin-1-yl)-2-(2,6-dioxopiperidin-3-yl)-6-fluoroisoindoline-1,3-dione (4)

To a solution of benzyl (1-((1-(2-(2,6-dioxopiperidin-3-yl)-6-fluoro-1,3-dioxoisoindolin-5-yl)azetidin-3-yl)methyl)piperidin-4-yl)carbamate (310 mg, 536.70 μmol) in CF₃CH₂OH (20 mL) was added Pd(OH)₂/C (100 mg, 20% purity) under N₂ atmosphere, the suspension was degassed and purged with H₂ several times. The mixture was stirred at 15° C. for 16 hours under H₂ (15 psi). LCMS showed the starting material was consumed completely and a peak (87%) with desired mass. The reaction mixture was filtered and washed with CF₃CH₂OH (100 mL). The filtrate was concentrated to afford 5-(3-((4-aminopiperidin-1-yl)methyl)azetidin-1-yl)-2-(2,6-dioxopiperidin-3-yl)-6-fluoroisoindoline-1,3-dione (200 mg) as a yellow solid, which was used directly. MS(M+H)⁺=444.2

### Step 3. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(1-((1-(2-(2,6-dioxopiperidin-3-yl)-6-fluoro-1,3-dioxoisoindolin-5-yl)azetidin-3-yl)methyl)piperidin-4-yl)-3-methoxybenzamide (Compound 162)

To a solution of 4-((7,7-difluoro-9-isopropyl-5-methyl-6-oxo-6,7,8,9-tetrahydro-5H-pyrimido[4,5-b][1, 4]diazepin-2-yl)amino)-3-methoxybenzoic acid (90 mg, 213.58 μmol) in DMF (3 mL) were added HATU (105.57 mg, 277.65 μmol) and DIPEA (82.81 mg, 640.73 μmol, 111.60 μL), the mixture was stirred at 15° C. for 15 minutes, then 5-(3-((4-aminopiperidin-1-yl)methyl)azetidin-1-yl)-2-(2,6-dioxopiperidin-3-yl)-6-fluoroisoindoline-1,3-dione (100 mg, 225.49 μmol) was added and the resulting mixture was stirred at 15° C. for 1 hour. LCMS showed 38% of 4-((7,7-difluoro-9-isopropyl-5-methyl-6-oxo-6,7,8,9-tetrahydro-5H-pyrimido[4,5-b][1, 4]diazepin-2-yl)amino)-3-methoxybenzoic acid remained and 22% of desired mass. To the mixture was added CH₃COOH to adjust pH<7. The resulting mixture was purified by prep-HPLC (column: Shim-pack C18 150*25*10 μm; mobile phase: [water (FA)-ACN]; B %: 10%-40%, 10 min) followed by prep-HPLC (column: Waters Xbridge 150*25 mm*5 μm; mobile phase: [water(NH₄HCO₃)-ACN]; B %: 43%-73%, 8 min), the eluent was freeze-dried to afford 4-((7,7-difluoro-9-isopropyl-5-methyl-6-oxo-6,7,8,9-tetrahydro-5H-pyrimido[4,5-b][1, 4]diazepin-2-yl)amino)-N-(1-((1-(2-(2,6-dioxopiperidin-3-yl)-6-fluoro-1,3-dioxoisoindolin-5-yl)azetidin-3-yl)methyl)piperidin-4-yl)-3-methoxybenzamide (8 mg, 8.97 μmol, 4.20% yield, 95% purity) as a yellow solid. MS(M+H)⁺=847.1.

¹H NMR (400 MHz, DMSO-d₆) δ=11.22-10.98 (m, 1H), 8.37-8.27 (m, 1H), 8.22 (s, 1H), 8.15-8.05 (m, 1H), 7.87 (s, 1H), 7.59 (d, J=11.2 Hz, 1H), 7.54-7.45 (m, 2H), 6.90 (d, J=7.6 Hz, 1H), 5.06 (dd, J=5.4, 12.7 Hz, 1H), 4.95-4.80 (m, 1H), 4.27 (br t, J=7.5 Hz, 2H), 4.04 (br t, J=13.6 Hz, 2H), 3.94 (s, 3H), 3.88-3.69 (m, 3H), 3.30 (s, 3H), 3.05-2.93 (m, 1H), 2.93-2.75 (m, 3H), 2.65-2.54 (m, 4H), 2.09-1.98 (m, 3H), 1.82-1.75 (m, 2H), 1.64-1.53 (m, 2H), 1.24 (d, J=6.7 Hz, 6H).

### Example 163. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-((3-(2-(2,6-dioxopiperidin-3-yl)-1,3-dioxoisoindolin-5-yl)azetidin-1-yl)methyl)piperidin-1-yl)-3-methoxybenzamide (Compound 163)

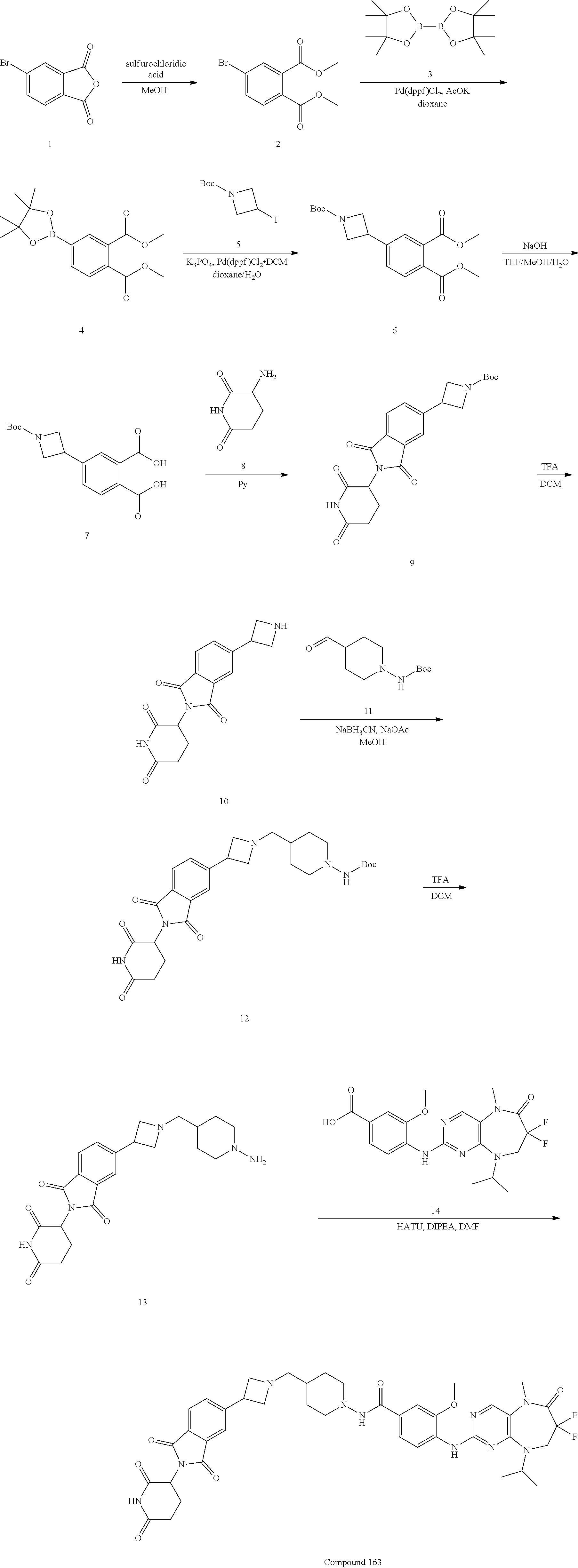

### Step 1. Synthesis of dimethyl 4-bromophthalate (2)

To a solution of 5-bromoisobenzofuran-1,3-dione (10 g, 44.05 mmol) in MeOH (100 mL) was added sulfurochloridic acid (1.75 g, 15.02 mmol, 1.00 mL) at 20° C. and the resulting mixture was stirred at 80° C. for 16 h. TLC (SiO₂, Petroleum ether:EtOAc=10:1) indicated starting material was consumed completely and one major new spot was formed. The reaction mixture was concentrated in vacuum. The residue was purified by flash silica gel chromatography (80 g SepaFlash® Silica Flash Column, Eluent of 0˜9% EtOAc/Petroleum ether gradient @100 mL/min) to afford dimethyl 4-bromophthalate (11.9 g, 43.58 mmol, 98.92% yield) as a yellow oil. MS(M+H)⁺=272.9

### Step 2. Synthesis of dimethyl 4-(4,4,5,5-tetramethyl-1,3,2-dioxaborolan-2-yl)phthalate (4)

To a solution of dimethyl 4-bromophthalate (11.9 g, 43.58 mmol) and 4,4,4′,4′,5,5,5′,5′-octamethyl-2,2′-bi(1,3,2-dioxaborolane) (13.28 g, 52.29 mmol) in dioxane (120 mL) were added Pd(dppf)Cl₂ (1.59 g, 2.18 mmol) and AcOK (8.55 g, 87.15 mmol) at 20° C. under N₂ atmosphere and the resulting mixture was stirred at 90° C. for 16 h under N₂ atmosphere. TLC (SiO₂, Petroleum ether:EtOAc=10:1) indicated starting material was consumed completely and one major new spot was formed. The reaction mixture was filtered and the filtrate was concentrated in vacuum. The residue was purified by flash silica gel chromatography (80 g SepaFlash® Silica Flash Column, Eluent of 0˜5% EtOAc/Petroleum ether gradient @100 mL/min) to afford dimethyl 4-(4,4,5,5-tetramethyl-1,3,2-dioxaborolan-2-yl)phthalate (15.1 g, crude) as a yellow oil. MS(M+H)⁺=321.1

### Step 3. Synthesis of dimethyl 4-(1-(tert-butoxycarbonyl)azetidin-3-yl)phthalate (6)

To a solution of dimethyl 4-(4,4,5,5-tetramethyl-1,3,2-dioxaborolan-2-yl)phthalate (4.6 g, 14.37 mmol) in H₂O (20 mL) and dioxane (60 mL) were added tert-butyl 3-iodoazetidine-1-carboxylate (4.88 g, 17.24 mmol), Pd(dppf)Cl₂ DCM (1.17 g, 1.44 mmol) and K₃PO₄ (9.15 g, 43.11 mmol) at 20° C. under N₂ atmosphere and the resulting mixture was stirred at 90° C. for 16 h under N₂ atmosphere. The reaction mixture was concentrated in vacuum. The residue was purified by flash silica gel chromatography (80 g SepaFlash® Silica Flash Column, Eluent of 0˜33% EtOAc/Petroleum ether gradient @100 mL/min) to afford dimethyl 4-(1-(tert-butoxycarbonyl)azetidin-3-yl)phthalate (2.3 g, 6.58 mmol, 45.82% yield) as a yellow oil. MS(M+H)⁺=350

### Step 4. Synthesis of 4-(1-(tert-butoxycarbonyl)azetidin-3-yl)phthalic acid (7)

To a solution of dimethyl 4-(1-(tert-butoxycarbonyl)azetidin-3-yl)phthalate (2.3 g, 6.58 mmol) in THF (6 mL) and MeOH (6 mL) and H₂O (6 mL) was added NaOH (1.32 g, 32.92 mmol) at 20° C. and the resulting mixture was stirred at 80° C. for 2 h. LCMS showed starting material was consumed completely. The reaction mixture was combined with another 2 batches (1.2 g and 0.5 g scale) for work-up. The reaction mixture was diluted with H₂O (20 mL) and washed with EtOAc (20 mL×3), the organic layers was discarded. The aqueous phase was adjusted the PH=6 with HCl (12 N) at 0° C., the resulting mixture was extracted with EtOAc (20 mL×3). The combined organic layer was dried over Na₂SO₄, filtered and concentrated to afford 4-(1-(tert-butoxycarbonyl)azetidin-3-yl)phthalic acid (2.3 g, 4.05 mmol, 61.46% yield) as a yellow oil. MS(M+H)⁺=322.1

### Step 5. Synthesis of tert-butyl 3-(2-(2,6-dioxopiperidin-3-yl)-1,3-dioxoisoindolin-5-yl)azetidine-1-carboxylate (9)

To a solution of 4-(1-(tert-butoxycarbonyl)azetidin-3-yl)phthalic acid (2.3 g, 7.16 mmol) in Py (20 mL) was added 3-aminopiperidine-2,6-dione (2.36 g, 14.32 mmol, HCl salt) at 20° C. and the resulting mixture was stirred at 120° C. for 16 h. LCMS showed starting material was consumed completely and a peak (48%) with desired mass. The reaction mixture was diluted with H₂O (20 mL) and extracted with EtOAc (20 mL×3). The organic layer was washed with brine (20 mL×3), dried over Na₂SO₄, filtered and concentrated. The residue was purified by flash silica gel chromatography (25 g SepaFlash® Silica Flash Column, Eluent of 0˜50% EtOAc/Petroleum ether gradient @100 mL/min) to afford tert-butyl 3-(2-(2,6-dioxopiperidin-3-yl)-1,3-dioxoisoindolin-5-yl)azetidine-1-carboxylate (1.3 g, 3.14 mmol, 43.93% yield) as an off-white solid. MS(M−56+H)⁺=358.3

### Step 6. Synthesis of 5-(azetidin-3-yl)-2-(2,6-dioxopiperidin-3-yl)isoindoline-1,3-dione (10)

To a solution of tert-butyl 3-(2-(2,6-dioxopiperidin-3-yl)-1,3-dioxoisoindolin-5-yl)azetidine-1-carboxylate (0.6 g, 1.45 mmol) in DCM (3 mL) was added TFA (827.41 mg, 7.26 mmol, 537.28 μL) at 20° C. and the resulting mixture was stirred at 20° C. for 12 h. LCMS showed starting material was consumed completely and a peak (68%) with desired mass. The reaction mixture was combined with another batch (0.2 g scale) for work-up. The combined reaction mixture was concentrated in vacuum to afford 5-(azetidin-3-yl)-2-(2,6-dioxopiperidin-3-yl)isoindoline-1,3-dione (1 g, crude, TFA salt) as a yellow oil. MS(M+H)⁺=314.4

### Step 7. Synthesis of tert-butyl (4-((3-(2-(2,6-dioxopiperidin-3-yl)-1,3-dioxoisoindolin-5-yl)azetidin-1-yl)methyl)piperidin-1-yl)carbamate (12)

To a solution of 5-(azetidin-3-yl)-2-(2,6-dioxopiperidin-3-yl)isoindoline-1,3-dione (1 g, 3.19 mmol) in MeOH (10 mL) were added NaOAc (1.57 g, 19.15 mmol) followed by tert-butyl (4-formylpiperidin-1-yl)carbamate (2.91 g, 12.77 mmol) at 20° C., after stirring 1 h. Then NaBH₃CN (601.73 mg, 9.58 mmol) was added slowly at 20° C. and the resulting mixture was stirred at 20° C. for 2 h. LCMS showed starting material was consumed completely and a peak (13%) with desired mass. The reaction mixture was concentrated in vacuum. The residue was purified by flash silica gel chromatography (12 g SepaFlash® Silica Flash Column, Eluent of 0˜100% EtOAc/Petroleum ether to 0˜60% Dichloromethane/Methanol gradient @100 mL/min) and re-purified by prep-HPLC (column: Shim-pack C18 150*25*10 μm; mobile phase: [water(TFA)-ACN]; B %: 8%-38%, 10 min), the eluent was lyophilized to afford tert-butyl (4-((3-(2-(2,6-dioxopiperidin-3-yl)-1,3-dioxoisoindolin-5-yl)azetidin-1-yl)methyl)piperidin-1-yl)carbamate (253 mg, 395.55 μmol, 12.39% yield, TFA salt) as a white solid. MS(M+H)⁺=526.3

### Step 8. Synthesis of 5-(1-((1-aminopiperidin-4-yl)methyl)azetidin-3-yl)-2-(2,6-dioxopiperidin-3-yl)isoindoline-1,3-dione (13)

To a solution of tert-butyl (4-((3-(2-(2,6-dioxopiperidin-3-yl)-1,3-dioxoisoindolin-5-yl)azetidin-1-yl)methyl)piperidin-1-yl)carbamate (128 mg, 243.53 μmol) in DCM (2 mL) was added TFA (138.84 mg, 1.22 mmol, 90.16 μL) at 20° C. and the resulting mixture was stirred at 20° C. for 1 h. LCMS showed starting material was consumed completely and a peak with desired mass. The reaction mixture was concentrated in vacuum to afford 5-(1-((1-aminopiperidin-4-yl)methyl)azetidin-3-yl)-2-(2,6-dioxopiperidin-3-yl)isoindoline-1,3-dione (186 mg, crude, TFA salt) as a yellow oil. MS(M+H)⁺=426.2

### Step 9. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-((3-(2-(2,6-dioxopiperidin-3-yl)-1,3-dioxoisoindolin-5-yl)azetidin-1-yl)methyl)piperidin-1-yl)-3-methoxybenzamide (Compound 163)

To a solution of 4-((7,7-difluoro-9-isopropyl-5-methyl-6-oxo-6,7,8,9-tetrahydro-5H-pyrimido[4,5-b][1, 4]diazepin-2-yl)amino)-3-methoxybenzoic acid (100 mg, 237.31 μmol) in DMF (2 mL) were added DIPEA (61.34 mg, 474.61 μmol, 82.67 μL) and HATU (99.25 mg, 261.04 μmol). The mixture was stirred at 20° C. for 10 min and a solution of 5-(1-((1-aminopiperidin-4-yl)methyl)azetidin-3-yl)-2-(2,6-dioxopiperidin-3-yl)isoindoline-1,3-dione (185.64 mg, 344.09 μmol, TFA salt) in DMF (2 mL) with DIPEA (122.68 mg, 949.22 μmol, 165.34 μL) was added and the resulting mixture was stirred at 20° C. for 1 h. LCMS showed starting material was consumed completely and a peak (38%) with desired mass. The reaction mixture was diluted with H₂O (15 mL) and extracted with EtOAc (15 mL×3). The organic layer was dried over Na₂SO₄, filtered and concentrated in vacuum. The residue was purified by prep-HPLC (column: Phenomenex Synergi C18 150*25 mm*10 μm; mobile phase: [water(FA)-ACN]; B %: 19%-39%, 10 min) followed by prep-TLC (SiO₂, DCM:MeOH=10:1), the residue was lyophilized to afford 4-((7,7-difluoro-9-isopropyl-5-methyl-6-oxo-6,7,8,9-tetrahydro-5H-pyrimido[4,5-b][1, 4]diazepin-2-yl)amino)-N-(4-((3-(2-(2,6-dioxopiperidin-3-yl)-1,3-dioxoisoindolin-5-yl)azetidin-1-yl)methyl)piperidin-1-yl)-3-methoxybenzamide (10.3 mg, 11.68 μmol, 23.60% yield, 95% purity, 0.2 FA salt) as a white solid. MS(M+H)⁺=829.0

¹H NMR (400 MHz, DMSO-d₆) δ=11.12 (s, 1H), 9.26 (s, 1H), 8.30 (br d, J=8.2 Hz, 1H), 8.21 (s, 1H), 8.15 (s, 0.2H), 7.96 (s, 1H), 7.93-7.83 (m, 3H), 7.46-7.39 (m, 2H), 5.15 (dd, J=5.3, 12.8 Hz, 1H), 4.92-4.82 (m, 1H), 4.03 (br t, J=13.5 Hz, 2H), 3.93 (s, 3H), 3.87-3.79 (m, 1H), 3.68 (br t, J=6.4 Hz, 2H), 3.32 (br s, 3H), 3.00 (br d, J=9.7 Hz, 2H), 2.96-2.82 (m, 2H), 2.74 (br t, J=9.8 Hz, 2H), 2.64-2.54 (m, 3H), 2.42 (br s, 2H), 2.11-1.95 (m, 2H), 1.74 (br d, J=9.2 Hz, 2H), 1.31 (br d, J=5.7 Hz, 2H), 1.25 (s, 3H), 1.23 (s, 3H).

### Example 164. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(3-((1-(2-(2,6-dioxopiperidin-3-yl)-1,3-dioxoisoindolin-4-yl)piperidin-4-yl)(methyl)amino)cyclobutyl)-3-methoxybenzamide (Compound 164)

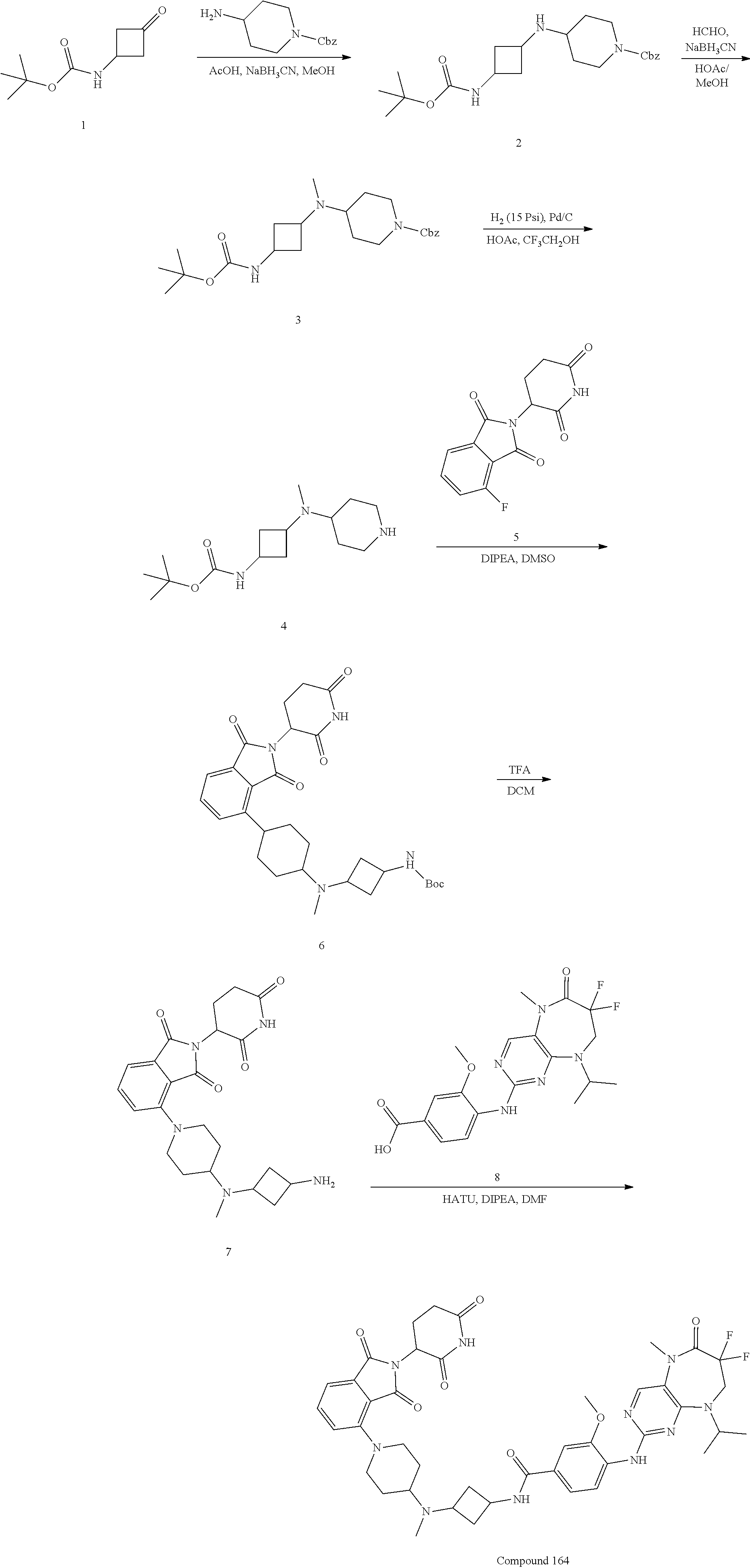

### Step 1. Synthesis of benzyl 4-((3-((tert-butoxycarbonyl)amino)cyclobutyl)amino)piperidine-1-carboxylate (2)

To a solution of tert-butyl (3-oxocyclobutyl)carbamate (1 g, 5.40 mmol) and benzyl 4-aminopiperidine-1-carboxylate (1.26 g, 5.40 mmol) in MeOH (15 mL) was added AcOH (324.22 mg, 5.40 mmol, 308.78 μL) at 20° C., then NaBH₃CN (1.02 g, 16.20 mmol) was slowly added at 20° C. and the resulting mixture was stirred at 20° C. for 12 hr. LCMS showed a peak (41%) with desired mass. The reaction mixture was diluted with H₂O (30 mL) and extracted with EtOAc (30 mL×3). The combined organic layers were washed with sat. NaHCO₃ (30 mL×3), dried over Na₂SO₄, filtered. The filtrate was concentrated to afford benzyl 4-((3-((tert-butoxycarbonyl)amino)cyclobutyl)amino)piperidine-1-carboxylate (2.1 g, 5.20 mmol, 96.39% yield) as a yellow oil. MS(M+H)⁺=404.2

### Step 2. Synthesis of benzyl 4-((3-((tert-butoxycarbonyl)amino)cyclobutyl)(methyl)amino)piperidine-1-carboxylate (3)

To a solution of benzyl 4-((3-((tert-butoxycarbonyl)amino)cyclobutyl)amino)piperidine-1-carboxylate (0.9 g, 2.23 mmol) in MeOH (10 mL) were added HOAc (133.94 mg, 2.23 mmol, 127.56 μL) and HCHO (362.00 mg, 4.46 mmol, 332.11 μL, 37% purity), the mixture was stirred at 20° C. for 30 min. Then NaBH₃CN (420.49 mg, 6.69 mmol) was added and the resulting mixture was stirred at 20° C. for 16 hr. LCMS showed the desired Mass. The reaction mixture was concentrated under reduced pressure. The residue was diluted with EtOAc (10 mL) and washed with H₂O (20 mL×3). The combined organic Layers were dried over Na₂SO₄, filtered and concentrated under reduce pressure to afford benzyl 4-((3-((tert-butoxycarbonyl)amino)cyclobutyl)(methyl)amino)piperidine-1-carboxylate (1.2 g, crude) as a colorless oil. MS(M+H)⁺=418.4

### Step 3. Synthesis of tert-butyl (3-(methyl(piperidin-4-yl)amino)cyclobutyl)carbamate (4)

To a solution of benzyl 4-((3-((tert-butoxycarbonyl)amino)cyclobutyl)(methyl)amino)piperidine-1-carboxylate (1.2 g, 2.87 mmol) in CF₃CH₂OH (10 mL) was added Pd/C (0.2 g, 187.93 μmol, 10% purity) under N₂ atmosphere, the mixture was degassed and purged with H₂ for 3 times, then the mixture was stirred at 20° C. for 16 h under H₂ (15 psi). LCMS showed most of starting material remained. HOAc (3 mL) was added, and the mixture was stirred at 20° C. for another 16 hr under H₂ (15 psi). LCMS showed that the desired Mass. The reaction mixture was filtered. The filtrate was concentrated under reduced pressure to afford tert-butyl (3-(methyl(piperidin-4-yl)amino)cyclobutyl)carbamate (1.6 g, crude) as a black solid. MS(M+H)⁺=284.4

### Step 4. Synthesis of tert-butyl (3-((1-(2-(2,6-dioxopiperidin-3-yl)-1,3-dioxoisoindolin-4-yl)piperidin-4-yl)(methyl) amino)cyclobutyl)carbamate (6)

To a solution of tert-butyl (3-(methyl(piperidin-4-yl)amino)cyclobutyl)carbamate (1.6 g, 5.65 mmol) in DMSO (10 mL) were added DIPEA (1.09 g, 8.47 mmol, 1.48 mL) and 2-(2,6-dioxopiperidin-3-yl)-4-fluoroisoindoline-1,3-dione (779.71 mg, 2.82 mmol), the mixture was stirred at 100° C. for 16 h. LCMS showed the desired Mass. The reaction mixture was diluted with H₂O (100 mL) and extracted with EtOAc (30 mL×3). The combined organic Layers were washed with brine (30 mL), dried over Na₂SO₄, filtered and concentrated under reduced pressure. The residue was purified by flash silica gel chromatography (ISCO®; 20 g SepaFlash® Silica Flash Column, Eluent of 50˜100% EtOAc/Petroleum ether to ˜100% MeOH/EtOAc gradient @100 mL/min) followed by prep-HPLC (column: Phenomenex Luna C18 150×40 mm×15 μm; mobile phase: [water(FA)-ACN]; B %: 8%-38%, 10 min), the eluent was lyophilized to afford tert-butyl (3-((1-(2-(2,6-dioxopiperidin-3-yl)-1,3-dioxoisoindolin-4-yl)piperidin-4-yl)(methyl)amino)cyclobutyl)carbamate (120 mg, 175.68 μmol, 6.22% yield, 79% purity, as a yellow solid. MS(M+H)⁺=540.5

### Step 5. Synthesis of 4-(4-((3-aminocyclobutyl)(methyl)amino)piperidin-1-yl)-2-(2,6-dioxopiperidin-3-yl)isoindoline-1,3-dione (7)

To a solution of tert-butyl (3-((1-(2-(2,6-dioxopiperidin-3-yl)-1,3-dioxoisoindolin-4-yl)piperidin-4-yl)(methyl)amino)cyclobutyl)carbamate (120 mg, 222.38 μmol) in DCM (1 mL) was added TFA (308.00 mg, 2.70 mmol, 0.2 mL) at 0° C., the mixture was stirred at 20° C. for 2 h. LCMS showed desired Mass. The reaction mixture was concentrated under reduced pressure to afford 4-(4-((3-aminocyclobutyl)(methyl)amino)piperidin-1-yl)-2-(2,6-dioxopiperidin-3-yl)isoindoline-1,3-dione (130 mg, crude, TFA) as a yellow oil. MS(M+H)⁺=440.4

### Step 6. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(3-((1-(2-(2,6-dioxopiperidin-3-yl)-1,3-dioxoisoindolin-4-yl)piperidin-4-yl)(methyl)amino)cyclobutyl)-3-methoxybenzamide (Compound 164)

A solution of 4-((7,7-difluoro-9-isopropyl-5-methyl-6-oxo-6,7,8,9-tetrahydro-5H-pyrimido[4,5-b][1, 4]diazepin-2-yl)amino)-3-methoxybenzoic acid (89.07 mg, 211.37 μmol), DIPEA (91.06 mg, 704.57 μmol, 122.72 μL) and HATU (133.95 mg, 352.28 μmol) in DMF (2 mL) was stirred at 20° C. for 20 min. Then 4-(4-((3-aminocyclobutyl)(methyl)amino)piperidin-1-yl)-2-(2,6-dioxopiperidin-3-yl)isoindoline-1,3-dione (130 mg, 234.86 μmol, TFA salt) and DIPEA (151.77 mg, 1.17 mmol, 204.54 μL) in DMF (2 mL) was added and the resulting mixture was stirred at 20° C. for 2 h. LCMS showed desired Mass. The reaction mixture was diluted with H₂O (50 mL) and extracted with EtOAc (20 mL×3). The combined organic Layers were washed with brine (20 mL), dried over Na₂SO₄, filtered and concentrated under reduced pressure. The residue was purified by prep-HPLC (column: Waters Xbridge 150×25 mm×5 μm; mobile phase: [water(NH₄HCO₃)-ACN]; B %: 42%-72%, 8 min) to afford 4-((7,7-difluoro-9-isopropyl-5-methyl-6-oxo-6,7,8,9-tetrahydro-5H-pyrimido[4,5-b][1, 4]diazepin-2-yl)amino)-N-(3-((1-(2-(2,6-dioxopiperidin-3-yl)-1,3-dioxoisoindolin-4-yl)piperidin-4-yl)(methyl)amino)cyclobutyl)-3-methoxybenzamide (31.3 mg, 35.28 μmol, 15.02% yield, 95% purity) as a yellow solid. MS(M+H)⁺=843.7

¹H NMR (400 MHz, DMSO-d₆) δ=11.08 (s, 1H), 8.43 (br d, J=7.8 Hz, 1H), 8.30 (br d, J=8.3 Hz, 1H), 8.22 (s, 1H), 7.87 (s, 1H), 7.68 (t, J=7.8 Hz, 1H), 7.56-7.49 (m, 2H), 7.37-7.29 (m, 2H), 5.10 (br dd, J=5.4, 12.8 Hz, 1H), 4.88 (td, J=6.8, 13.4 Hz, 1H), 4.21-4.12 (m, 1H), 4.03 (br t, J=13.6 Hz, 2H), 3.94 (s, 3H), 3.76 (br d, J=10.9 Hz, 2H), 3.29 (br s, 3H), 2.95-2.81 (m, 4H), 2.68-2.60 (m, 3H), 2.42 (br d, J=6.0 Hz, 2H), 2.10 (s, 3H), 2.07-2.00 (m, 1H), 1.99-1.89 (m, 2H), 1.70 (br s, 4H), 1.24 (br d, J=6.7 Hz, 6H).

### Example 165. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(3-((1-(2-(2,6-dioxopiperidin-3-yl)-1,3-dioxoisoindolin-4-yl)piperidin-4-yl)(methyl)amino)cyclopentyl)-3-methoxybenzamide (Compound 165)

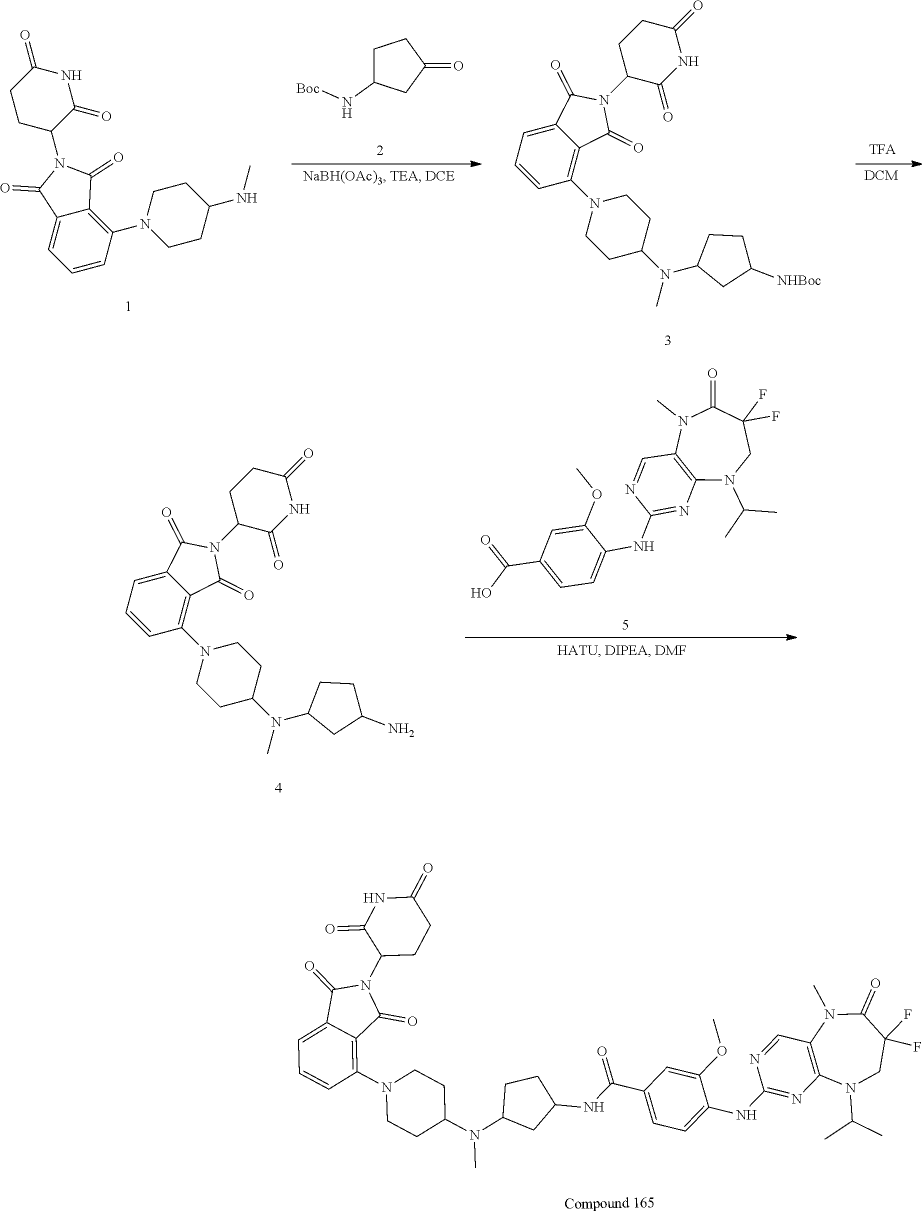 [SMILES: CNC1CCN(c2cccc3c2C(=O)N(C2CCC(=O)NC2=O)C3=O)CC1 ; CC(C)(C)OC(=O)NC1CCC(=O)C1 ; CN(C1CCN(c2cccc3c2C(=O)N(C2CCC(=O)NC2=O)C3=O)CC1)C1CCC(NC(=O)OC(C)(C)C)C1 ; CN(C1CCN(c2cccc3c2C(=O)N(C2CCC(=O)NC2=O)C3=O)CC1)C1CCC(N)C1 ; COc1cc(C(=O)O)ccc1Nc1ncc2c(n1)N(C(C)C)CC(F)(F)C(=O)N2C ; COc1cc(C(=O)NC2CCC(N(C)C3CCN(c4cccc5c4C(=O)N(C4CCC(=O)NC4=O)C5=O)CC3)C2)ccc1Nc1ncc2c(n1)N(C(C)C)CC(F)(F)C(=O)N2C]

### Step 1. Synthesis of tert-butyl (3-((1-(2-(2,6-dioxopiperidin-3-yl)-1,3-dioxoisoindolin-4-yl)piperidin-4-yl)(methyl) amino)cyclopentyl)carbamate (3)

To a solution of 2-(2,6-dioxopiperidin-3-yl)-4-(4-(methylamino)piperidin-1-yl)isoindoline-1,3-dione (120 mg, 294.94 μmol, HCl salt) and tert-butyl (3-oxocyclopentyl)carbamate (105.78 mg, 530.89 μmol) in DCE (2 mL) was added TEA (59.69 mg, 589.88 μmol, 82.10 μL). The mixture was stirred at 20° C. for 2 h. Then NaBH(OAc)₃ (187.53 mg, 884.82 μmol) was added, the mixture was stirred at 50° C. for 16 h. LCMS showed a peak (63%) with desired mass. The reaction mixture was quenched by addition of water (15 mL) at 0° C., and then extracted with dichloromethane (25 mL×2). The combined organic layers were washed with brine (15 mL), dried over Na₂SO₄, filtered and concentrated under reduced pressure, the residue was purified by flash silica gel chromatography (ISCO®; 12 g SepaFlash® Silica Flash Column, Eluent of 0˜100% MeOH/EtOAc@50 mL/min) to afford tert-butyl (3-((1-(2-(2,6-dioxopiperidin-3-yl)-1,3-dioxoisoindolin-4-yl)piperidin-4-yl)(methyl)amino)cyclopentyl)carbamate (160 mg, 288.99 μmol, 97.98% yield) as a yellow solid. MS(M+H)⁺=554.2

¹H NMR (400 MHz, CD₃OD) δ=7.72-7.65 (m, 1H), 7.41-7.39 (m, 1H), 7.34 (d, J=8.3 Hz, 1H), 5.11 (dd, J=5.4, 12.4 Hz, 1H), 4.04-4.00 (m, 1H), 3.95-3.85 (m, 2H), 3.83-3.73 (m, 1H), 3.05-2.93 (m, 2H), 2.89-2.59 (m, 7H), 2.50-2.37 (m, 1H), 2.28-1.98 (m, 8H), 1.95 (s, 2H), 1.89-1.79 (m, 1H), 1.74-1.51 (m, 2H), 1.44 (s, 9H)

### Step 2. Synthesis of 4-(4-((3-aminocyclopentyl)(methyl)amino)piperidin-1-yl)-2-(2,6-dioxopiperidin-3-yl)isoindoline-1,3-dione (4)

To a solution of tert-butyl (3-((1-(2-(2,6-dioxopiperidin-3-yl)-1,3-dioxoisoindolin-4-yl)piperidin-4-yl)(methyl)amino)cyclopentyl)carbamate (160 mg, 288.99 μmol) in DCM (1.5 mL) was added TFA (770.00 mg, 6.75 mmol, 500.00 μL). The mixture was stirred at 20° C. for 0.5 hr. LCMS one peak (80%) with desired mass. The reaction mixture was concentrated under reduced pressure to afford 4-(4-((3-aminocyclopentyl)(methyl)amino)piperidin-1-yl)-2-(2,6-dioxopiperidin-3-yl)isoindoline-1,3-dione (100 mg, 176.19 μmol, 60.97% yield, TFA salt) as a yellow solid. MS(M+H)⁺=454.3

### Step 3. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(3-((1-(2-(2,6-dioxopiperidin-3-yl)-1,3-dioxoisoindolin-4-yl)piperidin-4-yl)(methyl)amino)cyclopentyl)-3-methoxybenzamide (Compound 165)

To a solution of 4-((7,7-difluoro-9-isopropyl-5-methyl-6-oxo-6,7,8,9-tetrahydro-5H-pyrimido[4,5-b][1, 4]diazepin-2-yl)amino)-3-methoxybenzoic acid (75 mg, 177.98 μmol) and 4-(4-((3-aminocyclopentyl)(methyl)amino)piperidin-1-yl)-2-(2,6-dioxopiperidin-3-yl)isoindoline-1,3-dione (95.96 mg, 169.08 μmol, TFA salt) in DMF (1.5 mL) were added HATU (101.51 mg, 266.97 μmol,) and DIPEA (115.01 mg, 889.90 μmol, 155.00 μL). The mixture was stirred at 20° C. for 3 hr. LCMS showed one peak (39%) with desired mass. The reaction mixture was quenched by addition of water (15 mL) at 0° C., then extracted with EtOAc (30 mL×2). The combined organic layers were washed with brine (15 mL), dried over Na₂SO₄, filtered and concentrated under reduced pressure to give a residue, which was purified by prep-TLC (SiO₂, DCM:MeOH=10:1) followed by prep-HPLC (column: Waters Xbridge 150*25 mm*5 μm; mobile phase: [water (NH₄HCO₃)-ACN]; B %: 40%-70%, 8 min), and the eluent was lyophilized to afford 4-((7,7-difluoro-9-isopropyl-5-methyl-6-oxo-6,7,8,9-tetrahydro-5H-pyrimido[4,5-b][1, 4]diazepin-2-yl)amino)-N-(3-((1-(2-(2,6-dioxopiperidin-3-yl)-1,3-dioxoisoindolin-4-yl)piperidin-4-yl)(methyl)amino) cyclopentyl)-3-methoxybenzamide (13.7 mg, 14.87 μmol, 42.47% yield, 93% purity) as a yellow solid. MS(M+H)⁺=857.2

¹H NMR (400 MHz, DMSO-d₆) δ=11.09 (s, 1H), 8.37-8.29 (m, 1H), 8.25-8.17 (m, 2H), 7.92-7.83 (m, 1H), 7.73-7.62 (m, 1H), 7.55-7.44 (m, 2H), 7.38-7.28 (m, 2H), 5.14-5.03 (m, 1H), 4.94-4.83 (m, 1H), 4.33-4.31 (m, 1H), 4.09-3.99 (m, 2H), 3.94 (s, 3H), 3.79-3.72 (m, 2H), 3.30 (s, 3H), 2.91-2.84 (m, 5H), 2.77-2.75 (m, 2H), 2.17 (s, 3H), 2.05-2.03 (m, 2H), 1.96-1.85 (m, 2H), 1.79-1.71 (m, 4H), 1.63-1.40 (m, 3H), 1.28-1.23 (m, 6H).

### Example 166. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(3-((1-(2-(2,6-dioxopiperidin-3-yl)-1,3-dioxoisoindolin-4-yl)piperidin-4-yl)(methyl)amino)cyclohexyl)-3-methoxybenzamide (Compound 166)

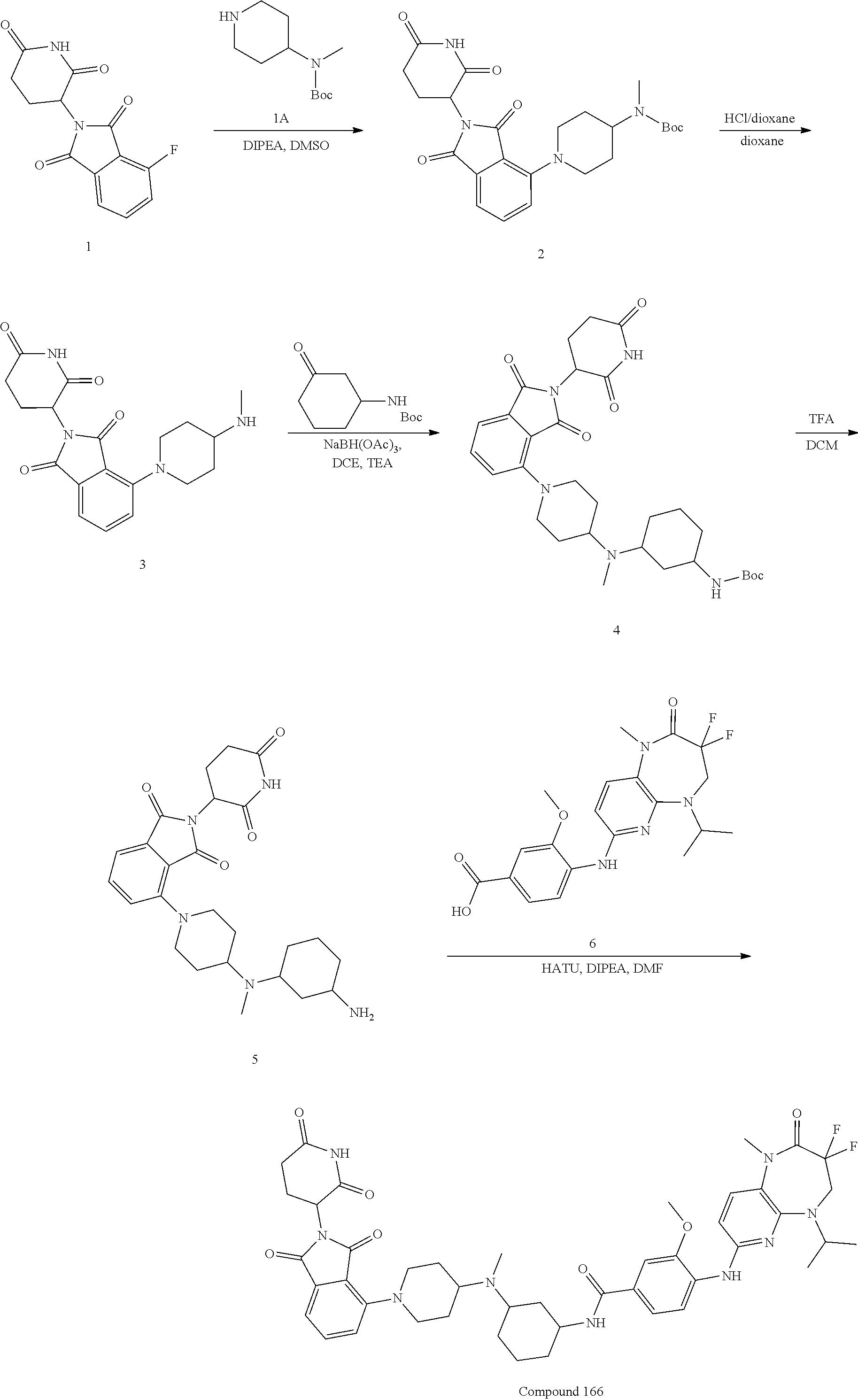

The compound 166 was synthesized by the method described in the scheme similar to the method described in Example 165.

MS(M+H)⁺=871.2, ¹H NMR (400 MHz, DMSO-d₆) δ=11.07 (s, 1H), 8.34-8.26 (m, 1H), 8.21 (s, 1H), 7.96 (d, J=7.1 Hz, 1H), 7.86 (s, 1H), 7.71-7.63 (m, 1H), 7.53-7.44 (m, 2H), 7.33 (t, J=7.0 Hz, 2H), 5.12-5.05 (m, 1H), 4.93-4.82 (m, 1H), 4.27-4.16 (m, 1H), 4.07-3.99 (m, 2H), 3.93 (s, 3H), 3.80-3.70 (m, 2H), 3.32-3.29 (m, 3H), 2.94-2.87 (m, 5H), 2.72-2.70 (m, 2H), 2.26-2.18 (m, 3H), 2.08-1.89 (m, 3H), 1.82-1.65 (m, 7H), 1.62-1.53 (m, 2H), 1.48-1.41 (m, 1H), 1.27-1.20 (m, 6H).

### Example 167. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(1-(2-(2,6-dioxopiperidin-3-yl)-1,3-dioxoisoindolin-4-yl)-3,3-difluoropiperidin-4-yl)piperazin-1-yl)-3-methoxybenzamide (Compound 167)

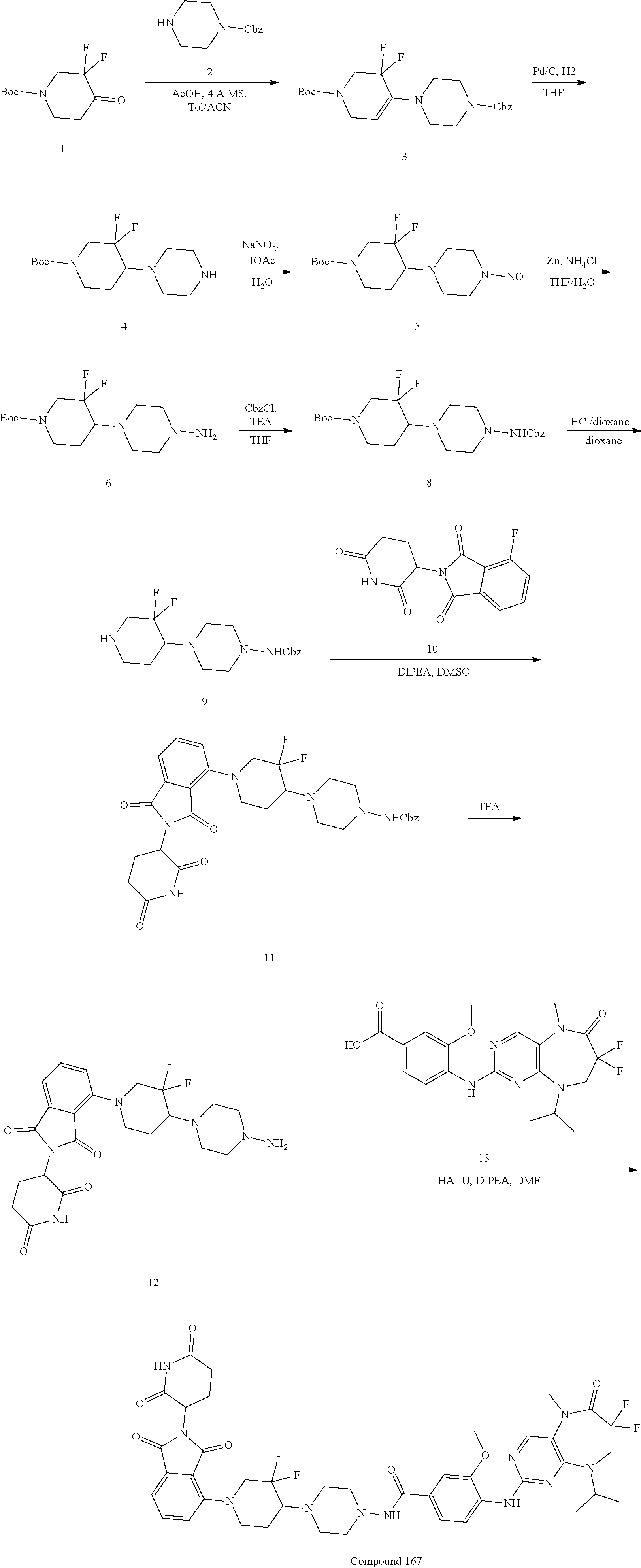

### Step 1. Synthesis of benzyl 4-(1-(tert-butoxycarbonyl)-3,3-difluoro-1,2,3,6-tetrahydropyridin-4-yl)piperazine-1-carboxylate (3)

To a solution of tert-butyl 3,3-difluoro-4-oxo-piperidine-1-carboxylate (4.7 g, 19.98 mmol) and benzyl piperazine-1-carboxylate (4.40 g, 19.98 mmol, 3.86 mL) in toluene (80 mL) and ACN (40 mL) were added AcOH (2.40 g, 39.96 mmol, 2.29 mL) and 4A MS (5 g) at 20° C. under N₂ and the resulting mixture was stirred at 120° C. for 12 h. LCMS showed all starting material was consumed completely and a peak (72%) with desired mass. The reaction mixture was filtered and the filtrate was concentrated in vacuum. The residue was purified by flash silica gel chromatography (40 g SepaFlash® Silica Flash Column, Eluent of 0˜33% EtOAc/Petroleum ether gradient @100 mL/min) to afford benzyl 4-(1-(tert-butoxycarbonyl)-3,3-difluoro-1,2,3,6-tetrahydropyridin-4-yl)piperazine-1-carboxylate (7.2 g, 16.46 mmol, 82.37% yield, 100% purity) as a white solid. MS(M+H)⁺=438.2

### Step 2. Synthesis of tert-butyl 3,3-difluoro-4-(piperazin-1-yl)piperidine-1-carboxylate (4)

To a solution of benzyl 4-(1-(tert-butoxycarbonyl)-3,3-difluoro-1,2,3,6-tetrahydropyridin-4-yl)piperazine-1-carboxylate (7.2 g, 16.46 mmol) in THF (50 mL) was added Pd/C (2 g, 10% purity) under N₂ atmosphere. The suspension was degassed and purged with H₂ for 3 times. The mixture was stirred under H₂ (15 Psi) at 20° C. for 12 h. LCMS showed starting material was consumed completely and a peak with desired mass. The reaction mixture was diluted with THF (280 mL) and filtered. The filtrate was concentrated in vacuum to afford tert-butyl 3,3-difluoro-4-(piperazin-1-yl)piperidine-1-carboxylate (5.5 g, crude) as a yellow oil. MS(M+H)⁺=306.4

### Step 3. Synthesis of tert-butyl 3,3-difluoro-4-(4-nitrosopiperazin-1-yl)piperidine-1-carboxylate (5)

To a solution of tert-butyl 3,3-difluoro-4-(piperazin-1-yl)piperidine-1-carboxylate (5.5 g, 18.01 mmol) in H₂O (60 mL) was added NaNO₂ (3.73 g, 54.03 mmol) at 0° C. Then AcOH (4.33 g, 72.05 mmol, 4.12 mL) was added drop-wise at 0° C. and the resulting mixture was stirred at 20° C. for 12 h. LCMS showed starting material was consumed completely and a main peak (93%) with mass (M−56). The pH of the reaction mixture was adjusted to 9 with saturated NaHCO₃ (60 mL), then extracted with EtOAc (120 mL×3). The organic layer was dried over Na₂SO₄, filtered and concentrated to afford tert-butyl 3,3-difluoro-4-(4-nitrosopiperazin-1-yl)piperidine-1-carboxylate (5.3 g, 15.85 mmol, 88.01% yield) as a yellow solid. MS(M−56+H)⁺=279.4

### Step 4. Synthesis of tert-butyl 4-(4-aminopiperazin-1-yl)-3,3-difluoropiperidine-1-carboxylate (6)

To a solution of tert-butyl 3,3-difluoro-4-(4-nitrosopiperazin-1-yl)piperidine-1-carboxylate (5.3 g, 15.85 mmol) in THF (50 mL) and H₂O (10 mL) was added NH₄Cl (3.39 g, 63.40 mmol) at 0° C. Then Zn (4.15 g, 63.40 mmol) was added portion wise at 0° C. and the resulting mixture was stirred at 20° C. for 12 h. LCMS showed starting material was consumed completely and a peak with desired mass. The reaction mixture was diluted with THF (60 mL) and filtered. The filtrate was concentrated in vacuum to afford tert-butyl 4-(4-aminopiperazin-1-yl)-3,3-difluoropiperidine-1-carboxylate (5.3 g, crude) as a yellow oil. MS(M+H)⁺=321.4

### Step 5. Synthesis of tert-butyl 4-(4-(((benzyloxy)carbonyl)amino)piperazin-1-yl)-3,3-difluoropiperidine-1-carboxylate (8)

To a solution of tert-butyl 4-(4-aminopiperazin-1-yl)-3,3-difluoropiperidine-1-carboxylate (5.3 g, 16.54 mmol) in THF (60 mL) were added TEA (8.37 g, 82.71 mmol, 11.51 mL) and CbzCl (4.23 g, 24.81 mmol, 3.53 mL) at 20° C. and the resulting mixture was stirred at 20° C. for 6 h. LCMS showed starting material was consumed completely and a peak (63%) with desired mass. The reaction mixture was diluted with H₂O (120 mL) and extracted with EtAOc (60 mL×3). The organic layer was dried over Na₂SO₄, filtered and concentrated. The residue was purified by flash silica gel chromatography (40 g SepaFlash® Silica Flash Column, Eluent of 0˜50% EtOAc/Petroleum ether gradient @100 mL/min) to afford tert-butyl 4-(4-(((benzyloxy)carbonyl)amino)piperazin-1-yl)-3,3-difluoropiperidine-1-carboxylate (3.2 g, 7.04 mmol, 42.56% yield) as a white solid. MS(M+H)⁺=455.3

### Step 6. Synthesis of benzyl (4-(3,3-difluoropiperidin-4-yl)piperazin-1-yl)carbamate (9)

To a solution of tert-butyl 4-(4-(((benzyloxy)carbonyl)amino)piperazin-1-yl)-3,3-difluoropiperidine-1-carboxylate (2 g, 4.40 mmol) in dioxane (10 mL) was added HCl/dioxane (4 M, 40 mL) at 20° C. and the resulting mixture was stirred at 20° C. for 0.5 h. LCMS showed starting material was consumed completely and a main peak (96%) with desired mass. The reaction mixture was concentrated in vacuum to afford benzyl (4-(3,3-difluoropiperidin-4-yl)piperazin-1-yl)carbamate (1.6 g, crude, HCl) as a white solid. MS(M+H)⁺=355.2

### Step 7. Synthesis of benzyl (4-(1-(2-(2,6-dioxopiperidin-3-yl)-1,3-dioxoisoindolin-4-yl)-3,3-difluoropiperidin-4-yl)piperazin-1-yl)carbamate (11)

To a solution of 2-(2,6-dioxo-3-piperidyl)-4-fluoro-isoindoline-1,3-dione (300 mg, 1.09 mmol) in DMSO (4 mL) were added DIPEA (561.47 mg, 4.34 mmol, 756.69 μL) and benzyl (4-(3,3-difluoropiperidin-4-yl)piperazin-1-yl)carbamate (424.50 mg, 1.09 mmol, HCl salt) at 20° C. and the resulting mixture was stirred at 120° C. for 36 h. LCMS showed starting material was consumed completely and a peak (40%) with desired mass. The reaction mixture was diluted with H₂O (15 mL) and extracted with EtOAc (15 mL×3). The organic layer was washed with brine (15 mL×3), dried over Na₂SO₄, filtered and concentrated. The residue was purified by flash silica gel chromatography (10 g SepaFlash® Silica Flash Column, Eluent of 0˜60% EtOAc/Petroleum ether gradient @100 mL/min) followed by prep-HPLC (column: Phenomenex Synergi Polar-RP 100*25 mm*4 um; mobile phase: [water(TFA)-ACN]; B %: 29%-49%, 7 min) and the eluent was lyophilized to afford benzyl (4-(1-(2-(2,6-dioxopiperidin-3-yl)-1,3-dioxoisoindolin-4-yl)-3,3-difluoropiperidin-4-yl)piperazin-1-yl)carbamate (68 mg, 111.36 μmol, 10.25% yield) as a yellow solid. MS(M+H)⁺=611.2

### Step 8. Synthesis of 4-(4-(4-aminopiperazin-1-yl)-3,3-difluoropiperidin-1-yl)-2-(2,6-dioxopiperidin-3-yl)isoindoline-1,3-dione (12)

A mixture of benzyl (4-(1-(2-(2,6-dioxopiperidin-3-yl)-1,3-dioxoisoindolin-4-yl)-3,3-difluoropiperidin-4-yl)piperazin-1-yl)carbamate (68 mg, 111.36 μmol) in TFA (3 mL) was stirred at 50° C. for 3 h. LCMS showed starting material was consumed completely and a peak (50%) with desired mass. The reaction mixture was concentrated in vacuum to afford 4-(4-(4-aminopiperazin-1-yl)-3,3-difluoropiperidin-1-yl)-2-(2,6-dioxopiperidin-3-yl)isoindoline-1,3-dione (58 mg, crude, HCl salt) as a yellow oil. MS(M+H)⁺=477.2

### Step 9. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(1-(2-(2,6-dioxopiperidin-3-yl)-1,3-dioxoisoindolin-4-yl)-3,3-difluoropiperidin-4-yl)piperazin-1-yl)-3-methoxybenzamide (Compound 167)

To a solution of 4-((7,7-difluoro-9-isopropyl-5-methyl-6-oxo-6,7,8,9-tetrahydro-5H-pyrimido[4,5-b][1, 4]diazepin-2-yl)amino)-3-methoxybenzoic acid (30 mg, 71.19 μmol) in DMF (1 mL) were added HATU (29.78 mg, 78.31 μmol) and DIPEA (18.40 mg, 142.38 μmol, 24.80 μL). The mixture was stirred at 20° C. for 10 min and a solution of 4-(4-(4-aminopiperazin-1-yl)-3,3-difluoropiperidin-1-yl)-2-(2,6-dioxopiperidin-3-yl)isoindoline-1,3-dione (54.65 mg, 92.55 μmol, TFA salt) in DMF (1 mL) with DIPEA (18.40 mg, 142.38 μmol, 24.80 μL) was added and the resulting mixture was stirred at 20° C. for 1 h. LCMS showed starting material was consumed completely and a peak (32%) with desired mass. The reaction mixture was diluted with H₂O (10 mL) and extracted with EtOAc (10 mL×3). The organic layer was washed with brine (10 mL×3), dried over Na₂SO₄, filtered and concentrated. The residue was purified by prep-HPLC (column: Phenomenex Synergi Polar-RP 100*25 mm*4 um; mobile phase: [water(TFA)-ACN]; B %: 28%-48%, 7 min) and prep-HPLC (column: Phenomenex C18 75*30 mm*3 um; mobile phase: [water(FA)-ACN]; B %: 22%-52%, 7 min) and the eluent was lyophilized to afford 4-((7,7-difluoro-9-isopropyl-5-methyl-6-oxo-6,7,8,9-tetrahydro-5H-pyrimido[4,5-b][1,4]diazepin-2-yl)amino)-N-(4-(1-(2-(2,6-dioxopiperidin-3-yl)-1,3-dioxoisoindolin-4-yl)-3,3-difluoropiperidin-4-yl)piperazin-1-yl)-3-methoxybenzamide (7.1 mg, 7.51 μmol, 10.55% yield, 95% purity, 0.4 FA) as a yellow solid. MS(M+H)⁺=880.3

¹H NMR (400 MHz, DMSO-d₆) δ=11.26-10.93 (m, 1H), 9.30 (s, 1H), 8.36-8.31 (m, 1H), 8.22 (s, 1H), 7.88 (s, 1H), 7.71 (t, J=7.8 Hz, 1H), 7.48-7.35 (m, 4H), 5.11 (dd, J=5.4, 12.8 Hz, 1H), 4.93-4.83 (m, 1H), 4.04 (br t, J=13.4 Hz, 2H), 3.98-3.83 (m, 5H), 3.32 (br s, 3H), 3.24-3.03 (m, 3H), 2.98-2.79 (m, 9H), 2.64-2.57 (m, 2H), 2.16-1.99 (m, 2H), 1.98-1.86 (m, 1H), 1.24 (d, J=6.7 Hz, 6H).

### Example 168. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(1-(2-(2,6-dioxopiperidin-3-yl)-1,3-dioxoisoindolin-5-yl)-3,3-difluoropiperidin-4-yl)piperazin-1-yl)-3-methoxybenzamide (Compound 168)

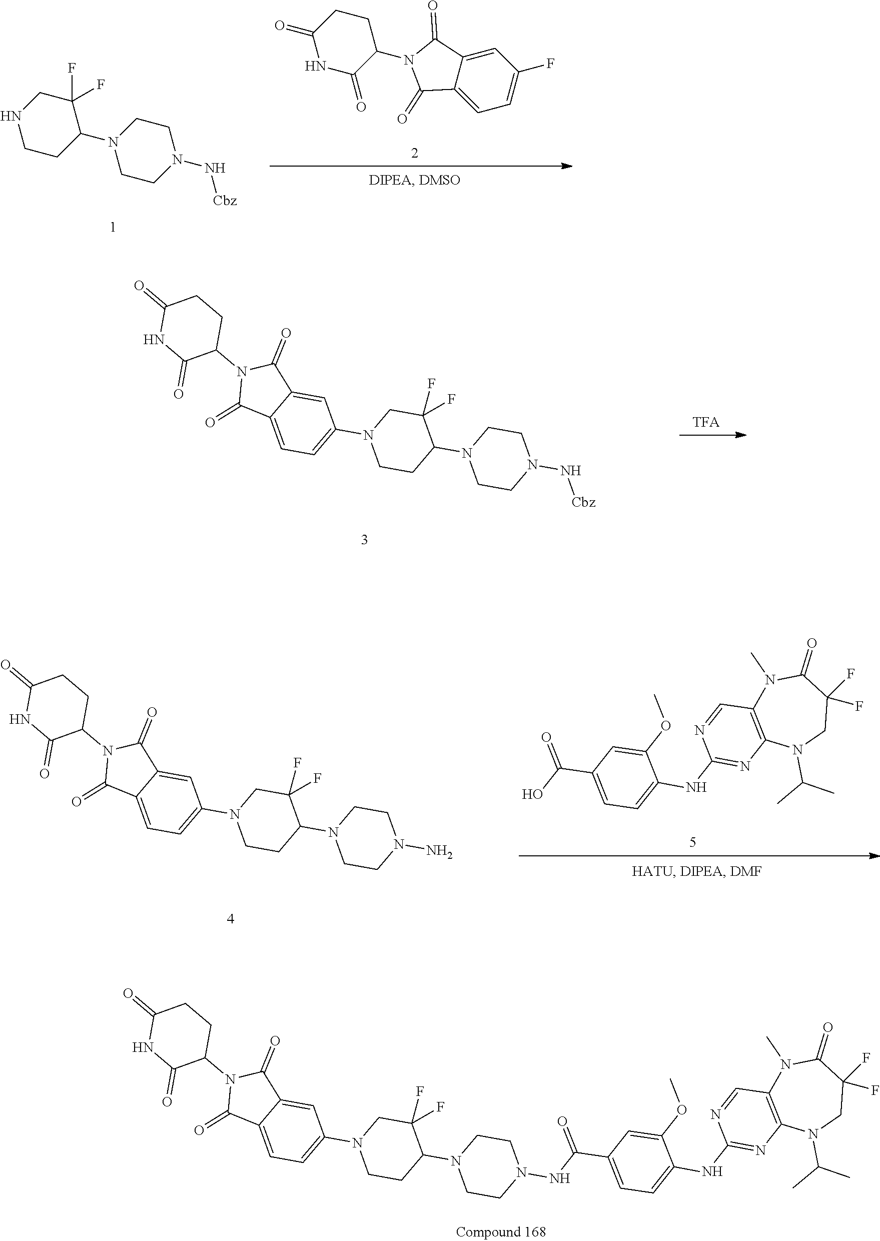 [SMILES: O=C(NN1CCN(C2CCNCC2(F)F)CC1)OCc1ccccc1 ; O=C1CCC(N2C(=O)c3ccc(F)cc3C2=O)C(=O)N1 ; O=C1CCC(N2C(=O)c3ccc(N4CCC(N5CCN(NC(=O)OCc6ccccc6)CC5)C(F)(F)C4)cc3C2=O)C(=O)N1 ; NN1CCN(C2CCN(c3ccc4c(c3)C(=O)N(C3CCC(=O)NC3=O)C4=O)CC2(F)F)CC1 ; COc1cc(C(=O)O)ccc1Nc1ncc2c(n1)N(C(C)C)CC(F)(F)C(=O)N2C ; COc1cc(C(=O)NN2CCN(C3CCN(c4ccc5c(c4)C(=O)N(C4CCC(=O)NC4=O)C5=O)CC3(F)F)CC2)ccc1Nc1ncc2c(n1)N(C(C)C)CC(F)(F)C(=O)N2C]

The compound 168 was synthesized by the method described in the scheme similar to the method described in Example 167.

MS(M+H)⁺=880.3, H NMR (400 MHz, DMSO-d₆) δ=11.08 (br s, 1H), 9.29 (s, 1H), 8.30 (br d, J=8.2 Hz, 1H), 8.22 (s, 1H), 7.88 (s, 1H), 7.68 (d, J=8.4 Hz, 1H), 7.43 (br d, J=11.4 Hz, 3H), 7.38-7.32 (m, 1H), 5.08 (dd, J=5.4, 12.8 Hz, 1H), 4.92-4.83 (m, 1H), 4.46-4.35 (m, 1H), 4.26-4.16 (m, 1H), 4.09-3.99 (m, 2H), 3.93 (s, 3H), 3.44-3.36 (m, 1H), 3.32 (s, 3H), 3.25-3.10 (m, 2H), 2.95-2.75 (m, 9H), 2.62-2.54 (m, 2H), 2.07-1.97 (m, 1H), 1.96-1.79 (m, 2H), 1.24 (d, J=6.7 Hz, 6H).

### Example 169. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-((2-(2,6-dioxopiperidin-3-yl)-1,3-dioxoisoindolin-4-yl)methyl)piperidin-4-yl)acetyl)piperidin-4-yl)-3-methoxybenzamide (Compound 169)

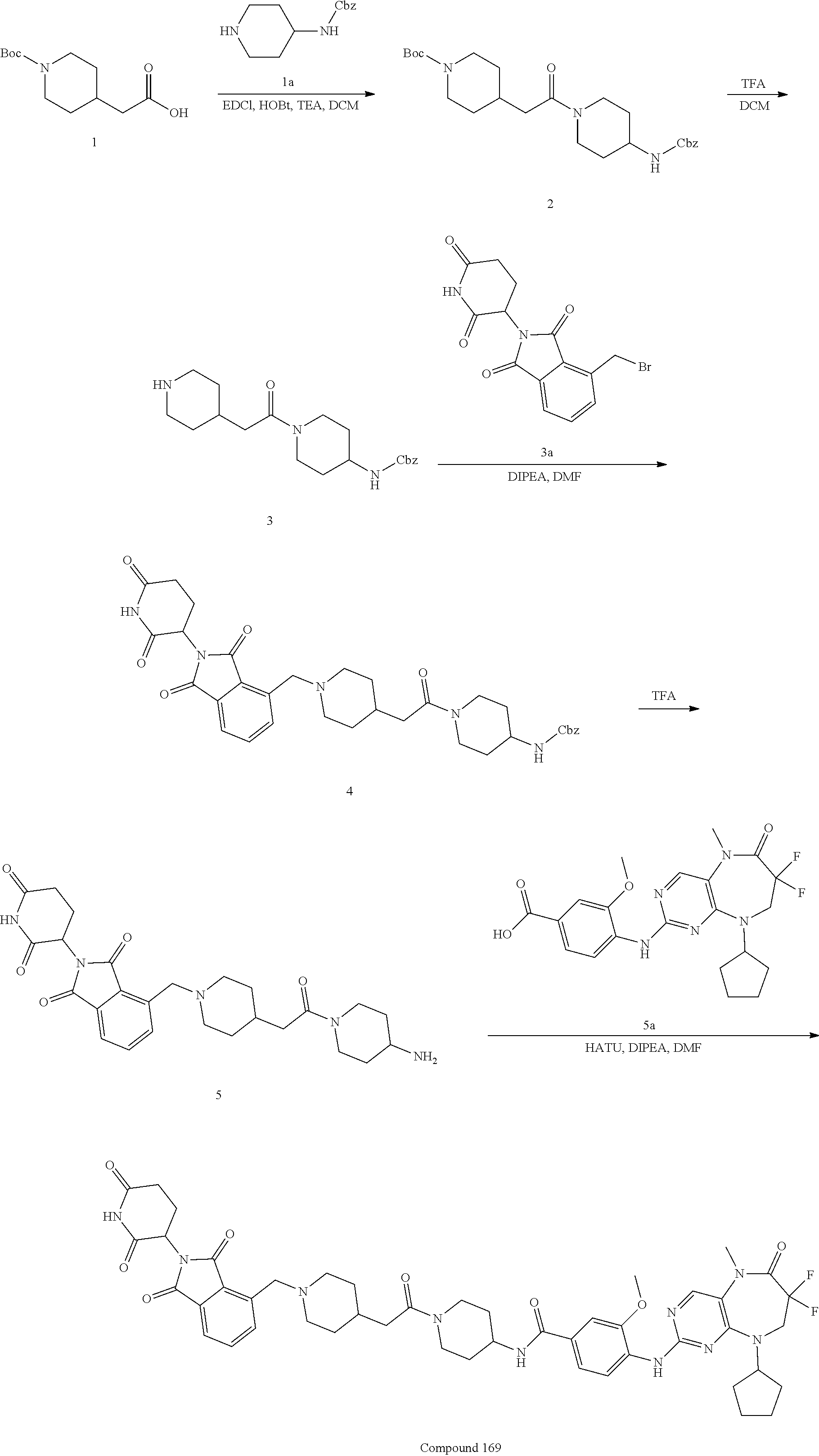

### Step 1. Synthesis of tert-butyl 4-(2-(4-(((benzyloxy)carbonyl)amino)piperidin-1-yl)-2-oxoethyl)piperidine-1-carboxylate (2)

To a mixture of 2-(1-(tert-butoxycarbonyl)piperidin-4-yl)acetic acid (0.96 g, 3.95 mmol) and benzyl piperidin-4-ylcarbamate (924.47 mg, 3.95 mmol) in DCM (30 mL) were added HOBt (586.47 mg, 4.34 mmol), EDCI (832.04 mg, 4.34 mmol) and TEA (1.20 g, 11.84 mmol, 1.65 mL) at 25° C. and the mixture was stirred at 25° C. for 12 h. LCMS showed a peak (92%) with desired mass. The mixture was washed with brine (10 mL×3), dried over Na₂SO₄, filtered and concentrated in vacuo. The residue was purified by flash silica gel chromatography (5 g SepaFlash® Silica Flash Column, Eluent of 0˜100% EtOAc/Petroleum ether gradient @60 mL/min) to afford tert-butyl 4-(2-(4-(((benzyloxy)carbonyl)amino)piperidin-1-yl)-2-oxoethyl)piperidine-1-carboxylate (1.6 g, 3.45 mmol, 87.35% yield, 99% purity) as yellow oil which was used for the next step directly. MS(M+H)⁺=460.2

### Step 2. Synthesis of benzyl (1-(2-(piperidin-4-yl)acetyl)piperidin-4-yl)carbamate (3)

To a solution of tert-butyl 4-(2-(4-(((benzyloxy)carbonyl)amino)piperidin-1-yl)-2-oxoethyl)piperidine-1-carboxylate (0.4 g, 870.37 μmol) in DCM (5 mL) was added TFA (1.54 g, 13.51 mmol, 1 mL) at 25° C. and the resulting mixture was stirred at 25° C. for 0.5 h. LCMS showed the starting material was consumed completely and a main peak with desired mass. The mixture solution was concentrated in vacuo at 30° C. to afford benzyl (1-(2-(piperidin-4-yl)acetyl)piperidin-4-yl)carbamate (0.4 g, 819.46 μmol, 94.15% yield, 97% purity, TFA) as yellow oil. MS(M+H)⁺=360.1

### Step 3. Synthesis of benzyl (1-(2-(1-((2-(2,6-dioxopiperidin-3-yl)-1,3-dioxoisoindolin-4-yl)methyl)piperidin-4-yl)acetyl)piperidin-4-yl)carbamate (4)

To a mixture of benzyl (1-(2-(piperidin-4-yl)acetyl)piperidin-4-yl)carbamate (372.07 mg, 939.76 μmol, TFA), DIPEA (441.67 mg, 3.42 mmol, 595.24 μL) in DMF (5 mL) was added 4-(bromomethyl)-2-(2,6-dioxopiperidin-3-yl)isoindoline-1,3-dione (300 mg, 854.33 μmol) at 25° C. and the mixture was stirred at 25° C. for 16 h. LCMS showed the 4-(bromomethyl)-2-(2,6-dioxopiperidin-3-yl)isoindoline-1,3-dione was consumed completely, and a peak (60%) with desired mass. The mixture was poured into water (10 mL) and extracted with EtOAc (20 mL×3). The combined organic layer was washed with brine (10 mL×3), dried over Na₂SO₄, filtered and concentrated in vacuo. The residue was purified by flash silica gel chromatography (5 g SepaFlash® Silica Flash Column, Eluent of 0˜100% EtOAc/Petroleum ether gradient @50 mL/min; Eluent of 0˜50% Methanol/EtOAc @50 mL/min) to afford benzyl (1-(2-(1-((2-(2,6-dioxopiperidin-3-yl)-1,3-dioxoisoindolin-4-yl)methyl)piperidin-4-yl) acetyl)piperidin-4-yl)carbamate (0.25 g, 381.13 μmol, 44.61% yield, 96% purity) as yellow oil. MS(M+H)⁺=630.2

### Step 4. Synthesis of 4-((4-(2-(4-aminopiperidin-1-yl)-2-oxoethyl)piperidin-1-yl)methyl)-2-(2,6-dioxopiperidin-3-yl)isoindoline-1,3-dione (5)

A solution of benzyl (1-(2-(1-((2-(2,6-dioxopiperidin-3-yl)-1,3-dioxoisoindolin-4-yl)methyl)piperidin-4-yl)acetyl)piperidin-4-yl)carbamate (0.25 g, 397.01 μmol) in TFA (3.08 g, 27.01 mmol, 2 mL) was stirred at 40° C. for 12 h. LCMS showed the starting material was consumed completely, and a peak (62%) with desired mass. The mixture solution was concentrated in vacuo at 40° C. to afford 4-((4-(2-(4-aminopiperidin-1-yl)-2-oxoethyl)piperidin-1-yl)methyl)-2-(2,6-dioxopiperidin-3-yl)isoindoline-1,3-dione (0.28 g, crude, TFA) as brown oil. MS(M+H)⁺=496.1

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-((2-(2,6-dioxopiperidin-3-yl)-1,3-dioxoisoindolin-4-yl)methyl)piperidin-4-yl)acetyl)piperidin-4-yl)-3-methoxybenzamide (Compound 169)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (80 mg, 178.80 μmol) in DMF (3 mL) were added HATU (88.38 mg, 232.44 μmol) and DIPEA (138.65 mg, 1.07 mmol, 186.86 μL) and the mixture was stirred at 25° C. for 10 min. To mixture was added 4-((4-(2-(4-aminopiperidin-1-yl)-2-oxoethyl)piperidin-1-yl)methyl)-2-(2,6-dioxopiperidin-3-yl)isoindoline-1,3-dione (196.19 mg, 321.83 μmol, TFA) and the resulting mixture was stirred at 25° C. for 12 h. LCMS showed the 4-((4-(2-(4-aminopiperidin-1-yl)-2-oxoethyl) piperidin-1-yl)methyl)-2-(2,6-dioxopiperidin-3-yl)isoindoline-1,3-dione was consumed completely and a peak (75%) with desired mass. The mixture solution was concentrated in vacuo. The residue was purified by flash silica gel chromatography (5 g SepaFlash® Silica Flash Column, Eluent of 0˜100% EtOAc/Petroleum ether gradient @50 mL/min; Eluent of 0˜50% Methanol/EtOAc @50 mL/min). The crude product was re-purified by prep-HPLC (column: Unisil 3-100 C18 ULtra 150×50 mm×3 um; mobile phase: [water(FA)-ACN]; B %: 10%-40%, 10 min) and the eluent lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-((2-(2,6-dioxopiperidin-3-yl)-1,3-dioxoisoindolin-4-yl)methyl)piperidin-4-yl)acetyl)piperidin-4-yl)-3-methoxybenzamide (37.9 mg, 38.11 μmol, 11.75% yield, 93% purity) as a white solid. MS(M+H)⁺=925.2

¹H NMR (400 MHz, DMSO-d₆) δ=11.12 (s, 1H), 8.30-8.22 (m, 2H), 8.18-8.10 (m, 1H), 7.96 (s, 1H), 7.92-7.87 (m, 1H), 7.87-7.77 (m, 2H), 7.51-7.43 (m, 2H), 5.14 (br d, J=5.5 Hz, 1H), 4.83-4.69 (m, 1H), 4.45-4.40 (m, 1H), 4.10-4.01 (m, 4H), 3.96-3.86 (m, 5H), 3.32 (br s, 3H), 3.13-3.06 (m, 1H), 2.94-2.80 (m, 3H), 2.72-2.55 (m, 3H), 2.55-2.51 (m, 2H), 2.31-2.20 (m, 2H), 2.12-2.02 (m, 2H), 1.99-1.90 (m, 2H), 1.89-1.76 (m, 2H), 1.73-1.64 (m, 4H), 1.63-1.50 (m, 4H), 1.50-1.34 (m, 2H), 1.32-1.19 (m, 2H).

### Example 170. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(1-((2-(2,6-dioxopiperidin-3-yl)-1,3-dioxoisoindolin-4-yl)methyl)piperidin-4-yl)propanoyl)piperidin-4-yl)-3-methoxybenzamide (Compound 170)

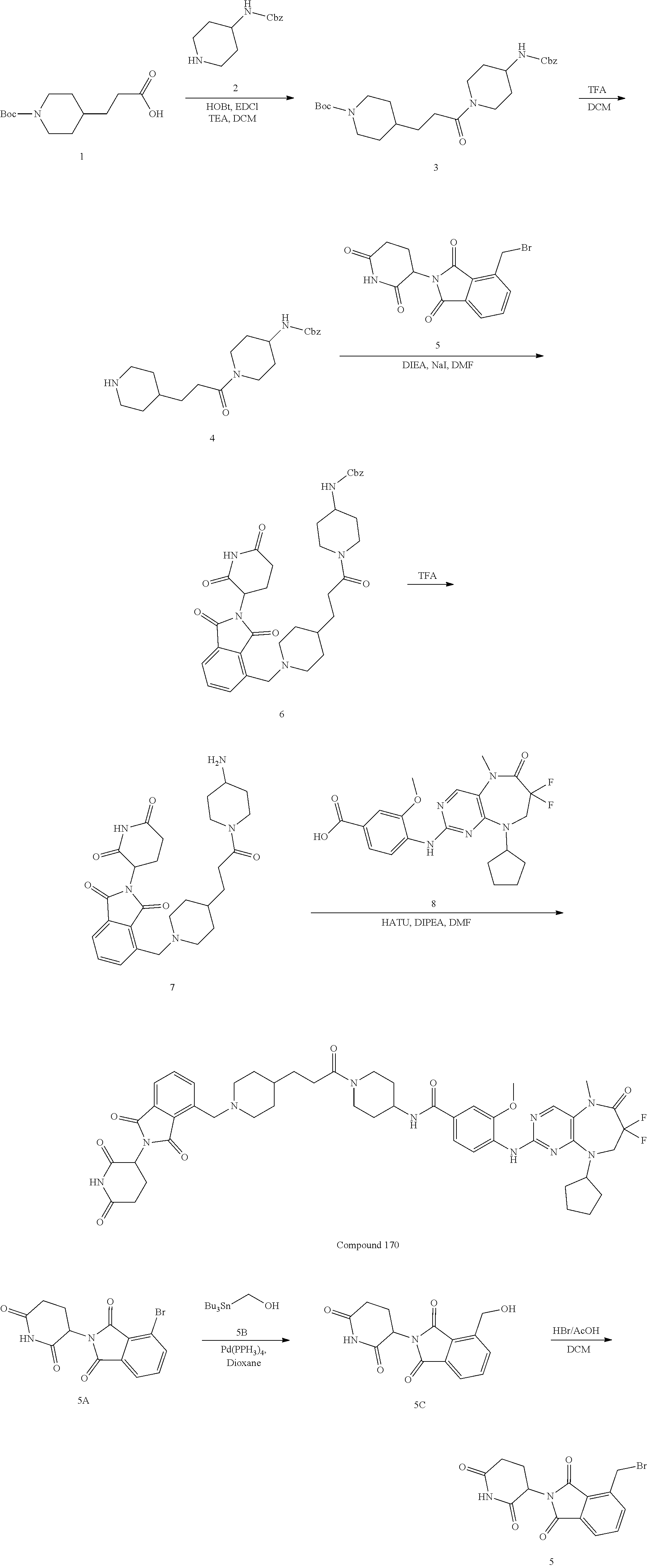

The compound 170 was synthesized by the method described in the scheme similar to the method described in Example 169.

MS(M+H)⁺=939.3, H NMR (400 MHz, DMSO-d₆) δ=11.12 (s, 1H), 8.33-8.22 (m, 2H), 8.14 (br d, J=7.6 Hz, 1H), 7.97 (s, 1H), 7.93-7.87 (m, 1H), 7.87-7.72 (m, 2H), 7.53-7.39 (m, 2H), 5.13 (dd, J=5.4, 12.7 Hz, 1H), 4.76 (quin, J=8.0 Hz, 1H), 4.38 (br d, J=12.8 Hz, 1H), 4.13-3.99 (m, 3H), 3.98-3.82 (m, 6H), 3.42 (br d, J=6.6 Hz, 3H), 3.11 (br t, J=12.1 Hz, 1H), 2.95-2.79 (m, 3H), 2.66-2.55 (m, 3H), 2.35-2.31 (m, 2H), 2.11-1.98 (m, 3H), 1.93 (br dd, J=3.7, 6.7 Hz, 2H), 1.89-1.78 (m, 2H), 1.74-1.63 (m, 4H), 1.63-1.50 (m, 4H), 1.50-1.33 (m, 4H), 1.28-1.12 (m, 3H).

### Example 171. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2R)-4-((2-(2,6-dioxopiperidin-3-yl)-1,3-dioxoisoindolin-4-yl)methyl)morpholin-2-yl)acetyl)piperidin-4-yl)-3-methoxybenzamide (Compound 171)

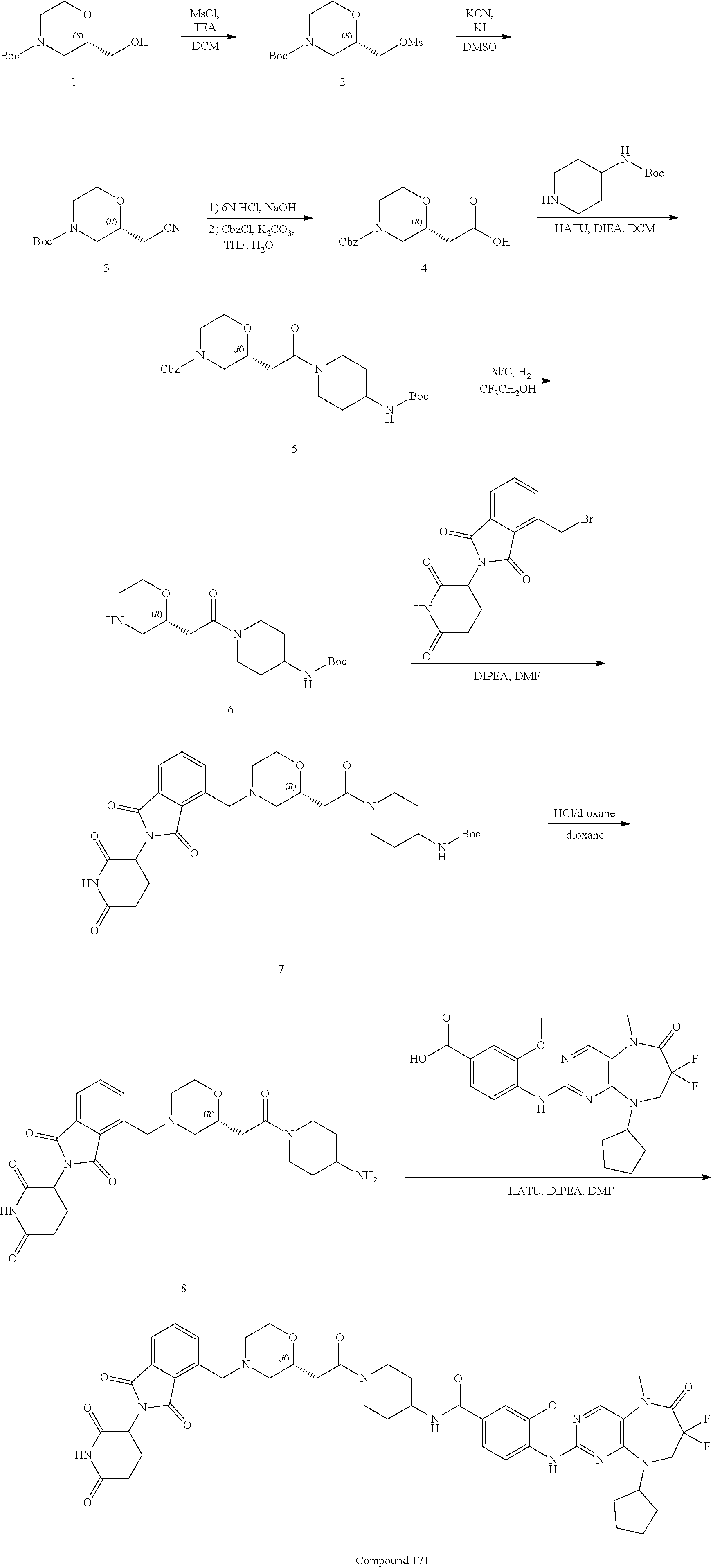

### Step 1. Synthesis of(S)-tert-butyl 2-(((methylsulfonyl)oxy)methyl)morpholine-4-carboxylate (2)

To a solution of (S)-tert-butyl 2-(hydroxymethyl)morpholine-4-carboxylate (5 g, 23.01 mmol) in DCM (50 mL) were added TEA (4.66 g, 46.03 mmol, 6.41 mL) and MsCl (3.43 g, 29.92 mmol, 2.32 mL) at 0° C. and the resulting mixture was stirred at 20° C. for 16 h. LCMS showed starting material was consumed completely and one peak with desired mass was detected. The reaction mixture poured into H₂O (50 mL) at 0° C. The aqueous phase was extracted with DCM (50 mL×3). The organic layer was dried over Na₂SO₄, filtered and concentrated to afford (S)-tert-butyl 2-(((methylsulfonyl)oxy)methyl)morpholine-4-carboxylate (7.1 g, crude) as an orange oil. MS(M−100+H)⁺=196.5

### Step 2. Synthesis of (R)-tert-butyl 2-(cyanomethyl)morpholine-4-carboxylate (3)

To a solution of (S)-tert-butyl 2-(((methylsulfonyl)oxy)methyl)morpholine-4-carboxylate (7.1 g, 24.04 mmol) in DMSO (80 mL) were added KI (5.99 g, 36.06 mmol) and KCN (1.64 g, 25.19 mmol, 1.08 mL) at 20° C. and the resulting mixture was stirred at 100° C. for 16 h. LCMS showed starting material was consumed completely and peak with desired mass (mass-99) was detected. The reaction mixture was diluted with H₂O (80 mL) and extracted with EtOAc (80 mL×3). The organic layer was washed with brine (80 mL×3), dried over Na₂SO₄, filtered and concentrated. The residue was purified by flash silica gel chromatography (40 g SepaFlash® Silica Flash Column, Eluent of 0˜33% EtOAc/Petroleum ether gradient @100 mL/min) to afford (R)-tert-butyl 2-(cyanomethyl)morpholine-4-carboxylate (4.1 g, 18.12 mmol, 75.38% yield) as a white solid. MS(M−100+H)⁺=127.7

### Step 3. Synthesis of (R)-2-(4-((benzyloxy)carbonyl)morpholin-2-yl)acetic acid (4)

A mixture of (R)-tert-butyl 2-(cyanomethyl)morpholine-4-carboxylate (4.1 g, 18.12 mmol) in HCl (6 M, 40 mL) at 20° C. and the resulting mixture was stirred at 100° C. for 12 h. LCMS showed starting material was consumed completely and added NaOH to this reaction mixture at 0° C. to adjust the pH=7. To a solution of the reaction mixture in THF (40 mL) and H₂O (30 mL) were added K₂CO₃ (7.51 g, 54.36 mmol) and CbzCl (4.64 g, 27.18 mmol, 3.86 mL) at 20° C. and the resulting mixture was stirred at 20° C. for 12 h. LCMS showed starting material was consumed completely and peak with desired mass was detected. The reaction mixture was extracted with EtOAc (30 mL×3). Added HCl (12 N) to this aqueous phase to adjust the pH=4 and extracted with EtOAc (30 mL×3). The organic layer was dried over Na₂SO₄, filtered and concentrated to afford (R)-2-(4-((benzyloxy)carbonyl)morpholin-2-yl)acetic acid (3.4 g, 11.32 mmol, 62.48% yield, 93% purity) as a yellow oil. SFC (retention time=1.153, method: “Column: Chiralpak AD-3 50×4.6 mm I. D. 3 um Mobile phase: Phase A for CO₂, and Phase B for MeOH (0.05% DEA); Gradient elution: MeOH (0.05% DEA) in CO₂ from 5% to 40% Flow rate: 3 mL/min; Detector: PDA; Column Temp: 35 C; Back Pressure: 100 Bar”). MS(M+H)⁺=280.4

### Step 4. Synthesis of (R)-benzyl 2-(2-(4-((tert-butoxycarbonyl)amino)piperidin-1-yl)-2-oxoethyl)morpholine-4-carboxylate (5)

To a solution of (R)-2-(4-((benzyloxy)carbonyl)morpholin-2-yl)acetic acid (600 mg, 2.15 mmol) in DCM (6 mL) was added HATU (898.54 mg, 2.36 mmol) and DIPEA (832.97 mg, 6.44 mmol, 1.12 mL). The mixture was stirred at 20° C. for 10 min and a solution of tert-butyl N-(4-piperidyl) carbamate (516.31 mg, 2.58 mmol) in DCM (6 mL) was added drop-wise at 20° C. and the resulting mixture was stirred at 20° C. for 1 h. LCMS showed starting material was consumed completely and peak with desired mass was detected. The reaction mixture was concentrated in vacuum. The residue was purified by flash silica gel chromatography (10 g SepaFlash® Silica Flash Column, Eluent of 0˜50% EtOAc/Petroleum ether gradient @100 mL/min) to afford (R)-benzyl 2-(2-(4-((tert-butoxycarbonyl)amino)piperidin-1-yl)-2-oxoethyl)morpholine-4-carboxylate (910 mg, 1.75 mmol, 81.68% yield, 89% purity) as a yellow oil. MS(M−100+H)⁺=362.2

### Step 5. Synthesis of (R)-tert-butyl (1-(2-(morpholin-2-yl)acetyl)piperidin-4-yl)carbamate (6)

To a solution of (R)-benzyl 2-(2-(4-((tert-butoxycarbonyl)amino)piperidin-1-yl)-2-oxoethyl)morpholine-4-carboxylate (900 mg, 1.95 mmol) in CF₃CH₂OH (10 mL) was added Pd/C (90 mg, 195.00 μmol, 19.50 μL, 10% purity) under N₂ atmosphere. The suspension was degassed and purged with H₂ for 3 times. The mixture was stirred under H₂ (15 Psi) at 20° C. for 16 h. LCMS showed starting material was consumed completely and peak with desired mass was detected. The reaction mixture was diluted with CF₃CH₂OH (30 mL) and filtered. The filtrate was concentrated in vacuum to afford (R)-tert-butyl (1-(2-(morpholin-2-yl)acetyl)piperidin-4-yl)carbamate (601 mg, crude) was obtained as a colorless oil. MS(M+H)⁺=328.4

### Step 6. Synthesis of tert-butyl (1-(2-((2R)-4-((2-(2,6-dioxopiperidin-3-yl)-1,3-dioxoisoindolin-4-yl)methyl)morpholin-2-yl)acetyl)piperidin-4-yl)carbamate (7)

To a solution of (R)-tert-butyl (1-(2-(morpholin-2-yl)acetyl)piperidin-4-yl)carbamate (341.26 mg, 1.04 mmol) in DMF (6 mL) were added DIPEA (336.77 mg, 2.61 mmol, 453.87 μL) and 4-(bromomethyl)-2-(2,6-dioxo-3-piperidyl) isoindoline-1,3-dione (305 mg, 868.57 μmol) at 20° C. and the resulting mixture was stirred at 20° C. for 12 h. LCMS showed starting material was consumed completely and one peak with desired mass was detected. The reaction mixture was diluted with H₂O (12 mL) and extracted with EtOAc (12 mL×3). The organic layer was washed with brine (12 mL×3), dried over Na₂SO₄, filtered and concentrated. The residue was purified by flash silica gel chromatography (4 g SepaFlash® Silica Flash Column, Eluent of 0˜100% EtOAc/Petroleum ether gradient @60 mL/min) to afford tert-butyl (1-(2-((2R)-4-((2-(2,6-dioxopiperidin-3-yl)-1,3-dioxoisoindolin-4-yl)methyl)morpholin-2-yl)acetyl)piperidin-4-yl)carbamate (410 mg, 651.71 μmol, 75.03% yield, 95% purity) as a yellow solid. MS(M+H)⁺=598.2

### Step 7. Synthesis of 4-(((R)-2-(2-(4-aminopiperidin-1-yl)-2-oxoethyl)morpholino)methyl)-2-(2,6-dioxopiperidin-3-yl)isoindoline-1,3-dione (8)

To a solution of tert-butyl (1-(2-((2R)-4-((2-(2,6-dioxopiperidin-3-yl)-1,3-dioxoisoindolin-4-yl)methyl)morpholin-2-yl)acetyl)piperidin-4-yl)carbamate (410 mg, 686.01 μmol) in dioxane (4 mL) was added HCl/dioxane (4 M, 12 mL) at 20° C. and the resulting mixture was stirred at 20° C. for 1 h. LCMS showed starting material was consumed completely and one peak with desired mass was detected. The reaction mixture was concentrated in vacuum to afford 4-(((R)-2-(2-(4-aminopiperidin-1-yl)-2-oxoethyl)morpholino)methyl)-2-(2,6-dioxopiperidin-3-yl)isoindoline-1,3-dione (402 mg, crude, HCl) as a white solid. MS(M+H)⁺=498.3

### Step 8. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2R)-4-((2-(2,6-dioxopiperidin-3-yl)-1,3-dioxoisoindolin-4-yl)methyl)morpholin-2-yl)acetyl)piperidin-4-yl)-3-methoxybenzamide (Compound 171)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (120 mg, 268.20 μmol) in DMF (3 mL) were added HATU (112.17 mg, 295.02 μmol) and DIPEA (69.32 mg, 536.39 μmol, 93.43 μL). The mixture was stirred at 20° C. for 10 min and a solution of 4-(((R)-2-(2-(4-aminopiperidin-1-yl)-2-oxoethyl)morpholino)methyl)-2-(2,6-dioxopiperidin-3-yl)isoindoline-1,3-dione (157.54 mg, 295.02 μmol, HCl) in DMF (3 mL) and DIPEA (69.32 mg, 536.39 μmol, 93.43 μL) was added and the resulting mixture was stirred at 20° C. for 1 h. LCMS showed starting material was consumed completely and one peak with desired mass was detected. The reaction mixture was diluted with H₂O (12 mL) and extracted with EtOAc (12 mL×3). The organic layer was dried over Na₂SO₄, filtered and concentrated. The residue was purified by prep-HPLC (column: Unisil 3-100 C₁₈ μLtra 150*50 mm*3 um; mobile phase: [water(FA)-ACN]; B %: 12%-42%, 10 min) and lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2R)-4-((2-(2,6-dioxopiperidin-3-yl)-1,3-dioxoisoindolin-4-yl)methyl)morpholin-2-yl)acetyl)piperidin-4-yl)-3-methoxybenzamide (87.8 mg, 92.82 μmol, 34.61% yield, 98% purity) as a white solid. MS(M+H)⁺=926.8

SFC (retention time: peak 1=0.729, peak 2=3.253; method: “Column: Chiralpak AD-3 50×4.6 mm I. D., 3 um Mobile phase: Phase A for CO₂, and Phase B for IPA+ACN (0.05% DEA); Gradient elution: 60% IPA+ACN (0.05% DEA) in CO₂Flow rate: 3 mL/min; Detector: PDAColumn Temp: 35 C; Back Pressure: 100 Bar”)

¹H NMR (400 MHz, DMSO-d₆) δ=11.12 (s, 1H), 8.23 (s, 1H), 8.15 (d, J=8.3 Hz, 1H), 7.99 (s, 1H), 7.92 (dd, J=1.3, 7.4 Hz, 1H), 7.88-7.78 (m, 3H), 7.02 (d, J=1.6 Hz, 1H), 6.94 (dd, J=1.7, 8.2 Hz, 1H), 5.13 (dd, J=5.4, 12.8 Hz, 1H), 4.75-4.66 (m, 1H), 4.57-4.09 (m, 1H), 4.02 (t, J=14.1 Hz, 2H), 3.95 (s, 2H), 3.88 (s, 3H), 3.86-3.67 (m, 4H), 3.54-3.46 (m, 1H), 3.32 (s, 3H), 3.16-2.96 (m, 2H), 2.92-2.80 (m, 1H), 2.79-2.72 (m, 1H), 2.70-2.52 (m, 3H), 2.29-2.22 (m, 1H), 2.19-2.11 (m, 2H), 2.09-2.01 (m, 1H), 1.95-1.84 (m, 3H), 1.79-1.62 (m, 4H), 1.62-1.47 (m, 4H), 1.38-1.20 (m, 2H).

### Example 172. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-((2S)-4-((2-(2,6-dioxopiperidin-3-yl)-1,3-dioxoisoindolin-4-yl)methyl)morpholin-2-yl)acetyl)piperidin-4-yl)-3-methoxybenzamide (Compound 172)

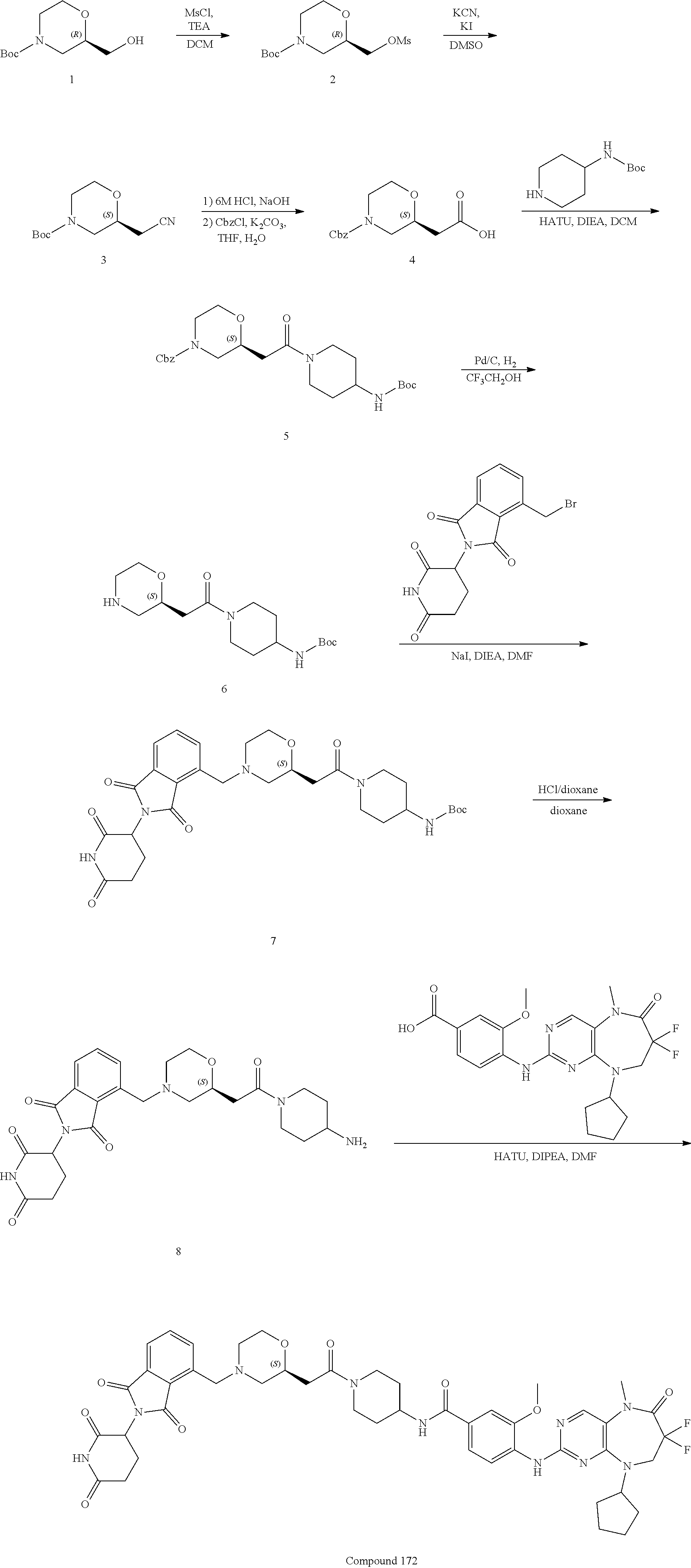

The compound 172 was synthesized by the method described in the scheme similar to the method described in Example 171.

MS(M+H)⁺=927.1, H NMR (400 MHz, DMSO-d₆) δ=11.12 (br s, 1H), 8.23 (s, 1H), 8.15 (d, J=8.2 Hz, 1H), 7.99 (s, 1H), 7.92 (d, J=7.3 Hz, 1H), 7.89-7.77 (m, 3H), 7.02 (d, J=1.5 Hz, 1H), 6.94 (dd, J=1.3, 8.3 Hz, 1H), 5.13 (dd, J=5.3, 12.8 Hz, 1H), 4.76-4.65 (m, 1H), 4.41-4.09 (m, 1H), 4.02 (t, J=14.1 Hz, 2H), 3.95 (s, 2H), 3.88 (s, 3H), 3.85-3.69 (m, 4H), 3.50 (dd, J=9.4, 11.2 Hz, 1H), 3.32 (s, 3H), 3.15-2.96 (m, 2H), 2.94-2.84 (m, 1H), 2.76 (br d, J=10.9 Hz, 1H), 2.69-2.60 (m, 2H), 2.56 (d, J=11.2 Hz, 1H), 2.29-2.21 (m, 1H), 2.19-2.10 (m, 2H), 2.08-2.05 (m, 1H), 1.95-1.85 (m, 3H), 1.79-1.63 (m, 4H), 1.62-1.51 (m, 4H), 1.38-1.21 (m, 2H).

### Example 173. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-((2R)-4-((2-(2,6-dioxopiperidin-3-yl)-1,3-dioxoisoindolin-4-yl)methyl)morpholin-2-yl)propanoyl)piperidin-4-yl)-3-methoxybenzamide (Compound 173)

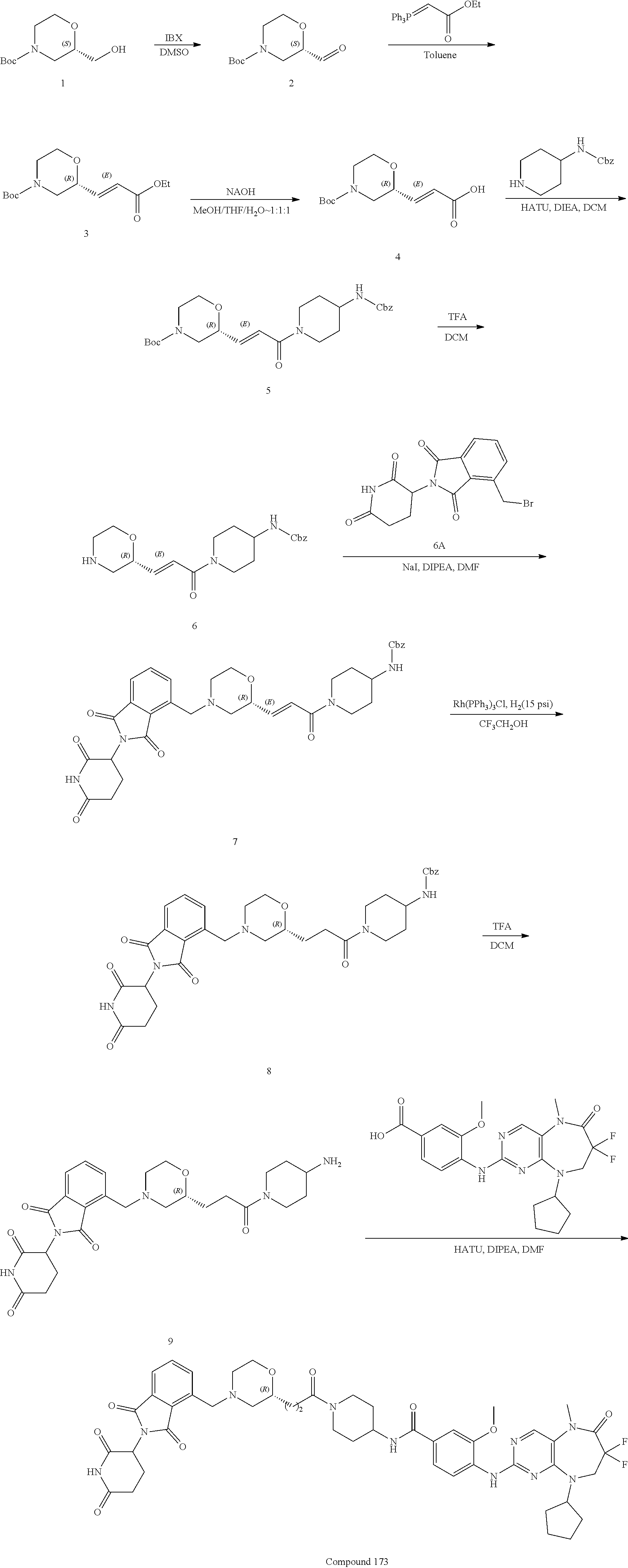

### Step 1. Synthesis of tert-butyl)-2-formylmorpholine-4-carboxylate (2)

To a solution of tert-butyl (2S)-2-(hydroxymethyl) morpholine-4-carboxylate (3 g, 13.81 mmol) in DMSO (40 mL) was added IBX (7.73 g, 27.62 mmol) at 20° C. and the resulting mixture was stirred at 20° C. for 16 h. TLC (SiO₂, EtOAc) indicated starting material was consumed completely and three new spots. The reaction mixture was combined with other scale (1 g) work-up. The reaction mixture was filtered. The filtrate was diluted with H₂O (30 mL) and extracted with EtOAc (30 mL×3). The organic layer was washed with brine (30 mL×3), dried over Na₂SO₄, filtered and concentrated to afford tert-butyl(S)-2-formylmorpholine-4-carboxylate (1.1 g, crude) as a yellow oil. MS (M+H)⁺=216.3

### Step 2. Synthesis of tert-butyl (R,E)-2-(3-ethoxy-3-oxoprop-1-en-1-yl)morpholine-4-carboxylate (3)

To a solution of tert-butyl (S)-2-formylmorpholine-4-carboxylate (1.1 g, 5.11 mmol) in toluene (10 mL) was added ethyl 2-(triphenyl-λ⁵-phosphanylidene)acetate (1.96 g, 5.62 mmol) at 20° C. and the resulting mixture was stirred at 90° C. for 16 h. TLC (SiO₂, Petroleum ether:EtOAc=5:1) indicated starting material was consumed completely and two new spots. The reaction mixture was combined with other scale (0.8 g) work-up. The reaction mixture was concentrated in vacuum. The residue was purified by flash silica gel chromatography (10 g SepaFlash® Silica Flash Column, Eluent of 0˜10% EtOAc/Petroleum ether gradient @80 mL/min) to afford tert-butyl (R,E)-2-(3-ethoxy-3-oxoprop-1-en-1-yl)morpholine-4-carboxylate (844 mg, 1.65 mmol, 32.37% yield) as a yellow oil. MS(M+H)⁺=286.3

### Step 3. Synthesis of (R,E)-3-(4-(tert-butoxycarbonyl)morpholin-2-yl)acrylic acid (4)

To a solution of tert-butyl (R,E)-2-(3-ethoxy-3-oxoprop-1-en-1-yl)morpholine-4-carboxylate (840 mg, 2.94 mmol) in MeOH (8 mL), THF (8 mL) and H₂O (8 mL) was added NaOH (1.18 g, 29.44 mmol) at 20° C. and the resulting mixture was stirred at 20° C. for 16 h. LCMS showed starting material was consumed completely and main peak with desired mass. Added HCl (12 M) to this reaction mixture to adjust the pH=5˜6. The aqueous phase was extracted with EtOAc (15 mL×4). The organic layer was dried over Na₂SO₄, filtered and concentrated to afford (R,E)-3-(4-(tert-butoxycarbonyl)morpholin-2-yl)acrylic acid (715 mg, 2.78 mmol, 94.40% yield) as a yellow oil. MS(M+Na)⁺=280.4

### Step 4. Synthesis of tert-butyl (R,E)-2-(3-(4-(((benzyloxy)carbonyl)amino)piperidin-1-yl)-3-oxoprop-1-en-1-yl)morpholine-4-carboxylate (5)

To a solution of (R,E)-3-(4-(tert-butoxycarbonyl)morpholin-2-yl)acrylic acid (400 mg, 1.55 mmol) in DCM (5 mL) were added HATU (650.26 mg, 1.71 mmol) and DIPEA (602.81 mg, 4.66 mmol, 812.41 μL). The mixture was stirred at 20° C. for 10 min and a solution of benzyl N-(4-piperidyl) carbamate (400.69 mg, 1.71 mmol) in DCM (5 mL) was added drop-wise at 20° C. and the resulting mixture was stirred at 20° C. for 1 h. LCMS showed all starting material was consumed completely and a peak (71%) with desired mass. The reaction mixture was concentrated in vacuum. The residue was purified by flash silica gel chromatography (20 g SepaFlash® Silica Flash Column, Eluent of 0˜60% EtOAc/Petroleum ether gradient @100 mL/min) to afford tert-butyl (R,E)-2-(3-(4-(((benzyloxy)carbonyl)amino)piperidin-1-yl)-3-oxoprop-1-en-1-yl) morpholine-4-carboxylate (482 mg, 916.04 μmol, 58.92% yield, 90% purity) as a yellow oil. SFC (retention time: 1.572; method: Column: Chiralcel OD-350×4.6 mm I.D., 3 um; Mobile phase: Phase A for CO₂, and Phase B for MEOH (0.05% DEA); Gradient elution: B in A from 5% to 40%; Flow rate: 3 mL/min; Detector: DAD; Column Temp: 35° C.; Back Pressure: 100 Bar). MS(M−56+H)⁺=418.3

### Step 5. Synthesis of benzyl (R,E)-(1-(3-(morpholin-2-yl)acryloyl)piperidin-4-yl)carbamate (6)

To a solution of tert-butyl (R,E)-2-(3-(4-(((benzyloxy)carbonyl)amino)piperidin-1-yl)-3-oxoprop-1-en-1-yl)morpholine-4-carboxylate (482 mg, 1.02 mmol) in DCM (10 mL) was added TFA (464.22 mg, 4.07 mmol, 301.44 μL) at 20° C. and the resulting mixture was stirred at 20° C. for 12 h. LCMS showed starting material was consumed completely and a peak (87%) with desired mass. The reaction mixture was concentrated in vacuum to afford benzyl (R,E)-(1-(3-(morpholin-2-yl)acryloyl)piperidin-4-yl)carbamate (496 mg, crude, TFA) as a yellow oil. MS(M+H)⁺=374.4

### Step 6. Synthesis of benzyl (1-((E)-3-((2R)-4-((2-(2,6-dioxopiperidin-3-yl)-1,3-dioxoisoindolin-4-yl)methyl)morpholin-2-yl)acryloyl)piperidin-4-yl)carbamate (7)

To a solution of 4-(bromomethyl)-2-(2,6-dioxopiperidin-3-yl)isoindoline-1,3-dione (100 mg, 284.78 μmol) in DMF (4 mL) were added DIPEA (184.03 mg, 1.42 mmol, 248.02 μL), NaI (8.54 mg, 56.96 μmol) and benzyl (R,E)-(1-(3-(morpholin-2-yl)acryloyl)piperidin-4-yl)carbamate (152.70 mg, 313.25 μmol, TFA) at 20° C. The mixture was stirred at 20° C. for 1 h. LCMS showed starting material was consumed completely and a peak (54%) with desired mass. The reaction mixture was diluted with H₂O (12 mL) and extracted with EtOAc (12 mL×3). The organic layer was washed with brine (12 mL×3), dried over Na₂SO₄, filtered and concentrated. The residue was purified by flash silica gel chromatography (4 g, SepaFlash® Silica Flash Column, Eluent of 0˜100% EtOAc/Petroleum ether gradient @60 mL/min) to afford benzyl (1-((E)-3-((2R)-4-((2-(2,6-dioxopiperidin-3-yl)-1,3-dioxoisoindolin-4-yl)methyl)morpholin-2-yl)acryloyl)piperidin-4-yl)carbamate (132 mg, 186.61 μmol, 66.73% yield, 91% purity) as a yellow solid. MS(M+H)⁺=644.1

### Step 7. Synthesis of benzyl (1-(3-((2R)-4-((2-(2,6-dioxopiperidin-3-yl)-1,3-dioxoisoindolin-4-yl)methyl)morpholin-2-yl)propanoyl)piperidin-4-yl)carbamate (8)

To a solution of benzyl (1-((E)-3-((2R)-4-((2-(2,6-dioxopiperidin-3-yl)-1,3-dioxoisoindolin-4-yl)methyl)morpholin-2-yl)acryloyl)piperidin-4-yl)carbamate (132 mg, 205.07 μmol) in CF₃CH₂OH (5 mL) was added Rh(PPh₃)₃Cl (189.73 mg, 205.07 μmol) under N₂ atmosphere. The suspension was degassed and purged with H₂ for 3 times and the resulting mixture was stirred under H₂ (15 Psi) at 20° C. for 16 h. LCMS showed starting material was consumed completely and a peak (23%) with desired mass was detected. The reaction mixture was concentrated in vacuum to afford benzyl (1-(3-((2R)-4-((2-(2,6-dioxopiperidin-3-yl)-1,3-dioxoisoindolin-4-yl)methyl)morpholin-2-yl)propanoyl)piperidin-4-yl)carbamate (202 mg, 96.98 μmol, 47.29% yield, 31% purity) as a yellow solid. MS(M+H)⁺=646.1

### Step 8. Synthesis of 4-(((R)-2-(3-(4-aminopiperidin-1-yl)-3-oxopropyl)morpholino)methyl)-2-(2,6-dioxopiperidin-3-yl)isoindoline-1,3-dione (9)

To a solution of benzyl (1-(3-((2R)-4-((2-(2,6-dioxopiperidin-3-yl)-1,3-dioxoisoindolin-4-yl)methyl)morpholin-2-yl)propanoyl)piperidin-4-yl)carbamate (202 mg, 96.98 μmol, 31% purity) in DCM (4 mL) was added TFA (110.58 mg, 969.80 μmol, 71.80 μL) at 20° C. and the resulting mixture was stirred at 50° C. for 32 h. LCMS showed starting material was consumed completely and a peak (12%) with desired mass. The reaction mixture was diluted with H₂O (10 mL) and extracted with EtOAc (10 mL×3). The aqueous phase was lyophilized to afford product A. The product A was purified by prep-HPLC (column: 3_Phenomenex Luna C18 75*30 mm*3 um; mobile phase: [water(HCl)-ACN]; B %: 0%-12%, 7 min) and the eluent was lyophilized to afford 4-(((R)-2-(3-(4-aminopiperidin-1-yl)-3-oxopropyl)morpholino)methyl)-2-(2,6-dioxopiperidin-3-yl)isoindoline-1,3-dione (15 mg, 26.82 μmol, 35.70% yield, 98% purity, HCl) as a white solid. MS(M+H)⁺=512.2

### Step 9. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-((2R)-4-((2-(2,6-dioxopiperidin-3-yl)-1,3-dioxoisoindolin-4-yl)methyl)morpholin-2-yl)propanoyl)piperidin-4-yl)-3-methoxybenzamide (Compound 173)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (10 mg, 22.35 μmol) in DMF (1 mL) were added HATU (9.35 mg, 24.58 μmol) and DIPEA (5.78 mg, 44.70 μmol, 7.79 μL). The mixture was stirred at 20° C. for 10 min and a solution of 4-(((R)-2-(3-(4-aminopiperidin-1-yl)-3-oxopropyl)morpholino)methyl)-2-(2,6-dioxopiperidin-3-yl)isoindoline-1,3-dione (12.25 mg, 22.35 μmol, HCl) in DMF (1 mL) with DIPEA (5.78 mg, 44.70 μmol, 7.79 μL) was added and the resulting mixture was stirred at 20° C. for 1 h. LCMS showed all starting material was consumed completely and a peak (71%) with desired mass. The reaction mixture was diluted with H₂O (8 mL) and extracted with EtOAc (8 mL×3). The organic layer was washed with brine (8 mL×3), dried over Na₂SO₄, filtered and concentrated. The residue was purified by prep-HPLC (column: Phenomenex Synergi C18 150*25 mm*10 um; mobile phase: [water(FA)-ACN]; B %: 14%-44%, 10 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-((2R)-4-((2-(2,6-dioxopiperidin-3-yl)-1,3-dioxoisoindolin-4-yl)methyl)morpholin-2-yl)propanoyl)piperidin-4-yl)-3-methoxybenzamide (7.8 mg, 7.93 μmol, 35.48% yield, 98% purity, 0.5 FA) as a white solid. SFC (retention time: peak 1=0.595, peak 2=2.530; method: “Column: Chiralpak AD-3 50×4.6 mm I.D., 3 um Mobile phase: Phase A for CO₂, and Phase B for IPA+ACN (0.05% DEA); Gradient elution: 60% IPA+ACN (0.05% DEA) in CO₂Flow rate: 3 mL/min; Detector: PDAColumn Temp: 35° C.; Back Pressure: 100 Bar”). MS(M+H)⁺=941.0.

¹H NMR (400 MHz, DMSO-d₆) δ=11.21-10.99 (m, 1H), 8.45-8.38 (m, 1H), 8.30-8.24 (m, 2H), 8.13 (br dd, J=3.1, 7.4 Hz, 1H), 7.96 (s, 1H), 7.94-7.90 (m, 1H), 7.87-7.80 (m, 2H), 7.51-7.43 (m, 2H), 5.13 (dd, J=5.4, 12.8 Hz, 1H), 4.82-4.71 (m, 1H), 4.39-4.35 (m, 1H), 4.09-4.01 (m, 3H), 3.98-3.91 (m, 5H), 3.91-3.83 (m, 1H), 3.79-3.73 (m, 1H), 3.54-3.38 (m, 5H), 3.14-3.05 (m, 1H), 2.94-2.83 (m, 1H), 2.82-2.76 (m, 1H), 2.65-2.53 (m, 4H), 2.43-2.36 (m, 2H), 2.20-2.11 (m, 1H), 2.10-2.01 (m, 1H), 1.98-1.77 (m, 5H), 1.75-1.66 (m, 2H), 1.65-1.52 (m, 6H), 1.50-1.31 (m, 2H).

### Example 174. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-((2S)-4-((2-(2,6-dioxopiperidin-3-yl)-1,3-dioxoisoindolin-4-yl)methyl)morpholin-2-yl)propanoyl)piperidin-4-yl)-3-methoxybenzamide (Compound 174)

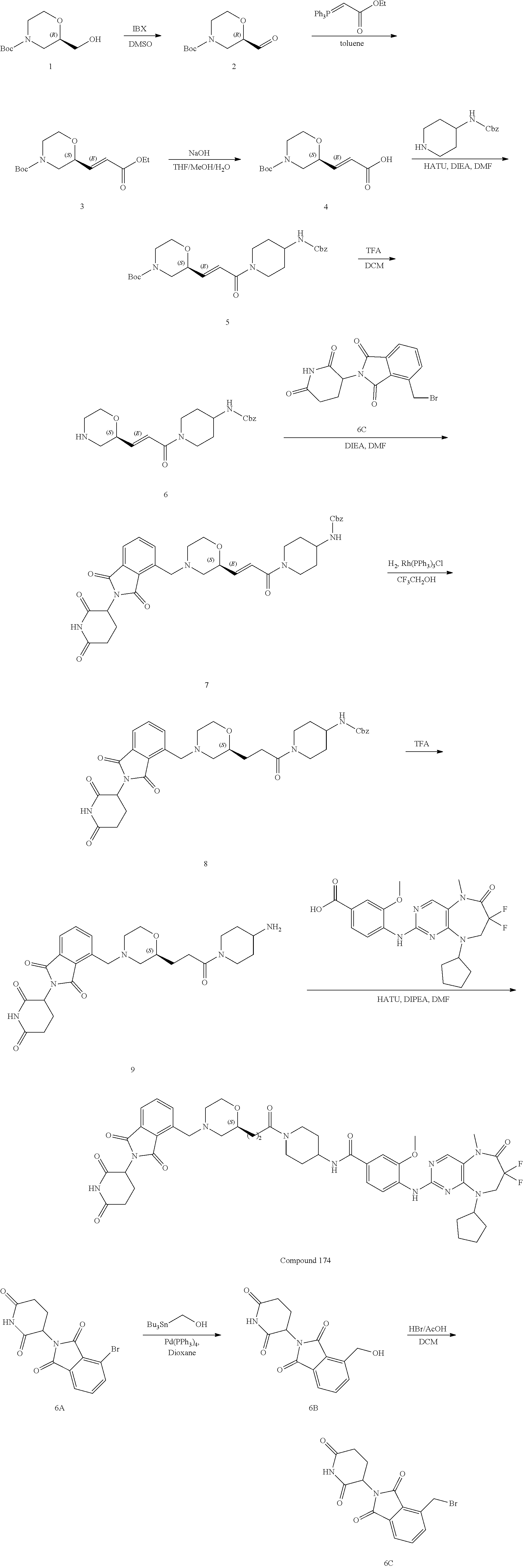

The compound 174 was synthesized by the method described in the scheme similar to the method described in Example 173.

MS(M+H)⁺=941.4, ¹H NMR (400 Hz, DMSO-d₆) δ=11.13 (br s, 1H), 8.30-8.25 (m, 2H), 8.15 (br d, J=3.4 Hz, 1H), 7.98 (s, 1H), 7.93 (br d, J=7.2 Hz, 1H), 7.84 (q, J=7.5 Hz, 2H), 7.50-7.46 (m, 2H), 5.14 (dd, J=5.3, 12.8 Hz, 1H), 4.79-4.76 (m, 1H), 4.40-4.33 (m, 1H), 4.11-3.98 (m, 4H), 3.97-3.92 (m, 2H), 3.94 (s, 3H), 3.89-3.87 (m, 1H), 3.79-3.77 (m, 1H), 3.52-3.41 (m, 1H), 3.32 (br s, 3H), 3.33-3.24 (m, 1H), 3.11-3.09 (m, 1H), 2.94-2.85 (m, 1H), 2.79-2.76 (m, 1H), 2.70-2.63 (m, 4H), 2.40-2.38 (m, 2H), 2.17-2.04 (m, 2H), 1.99-1.77 (m, 6H), 1.73-1.71 (m, 2H), 1.60-1.57 (m, 4H), 1.47-1.36 (m, 2H).

### Example 175. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(6-(2-(2,6-dioxopiperidin-3-yl)-1,3-dioxoisoindolin-4-yl)-2,6-diazaspiro[3.3]heptan-2-yl)propanoyl)piperidin-4-yl)-2-fluoro-5-methoxybenzamide (Compound 175)

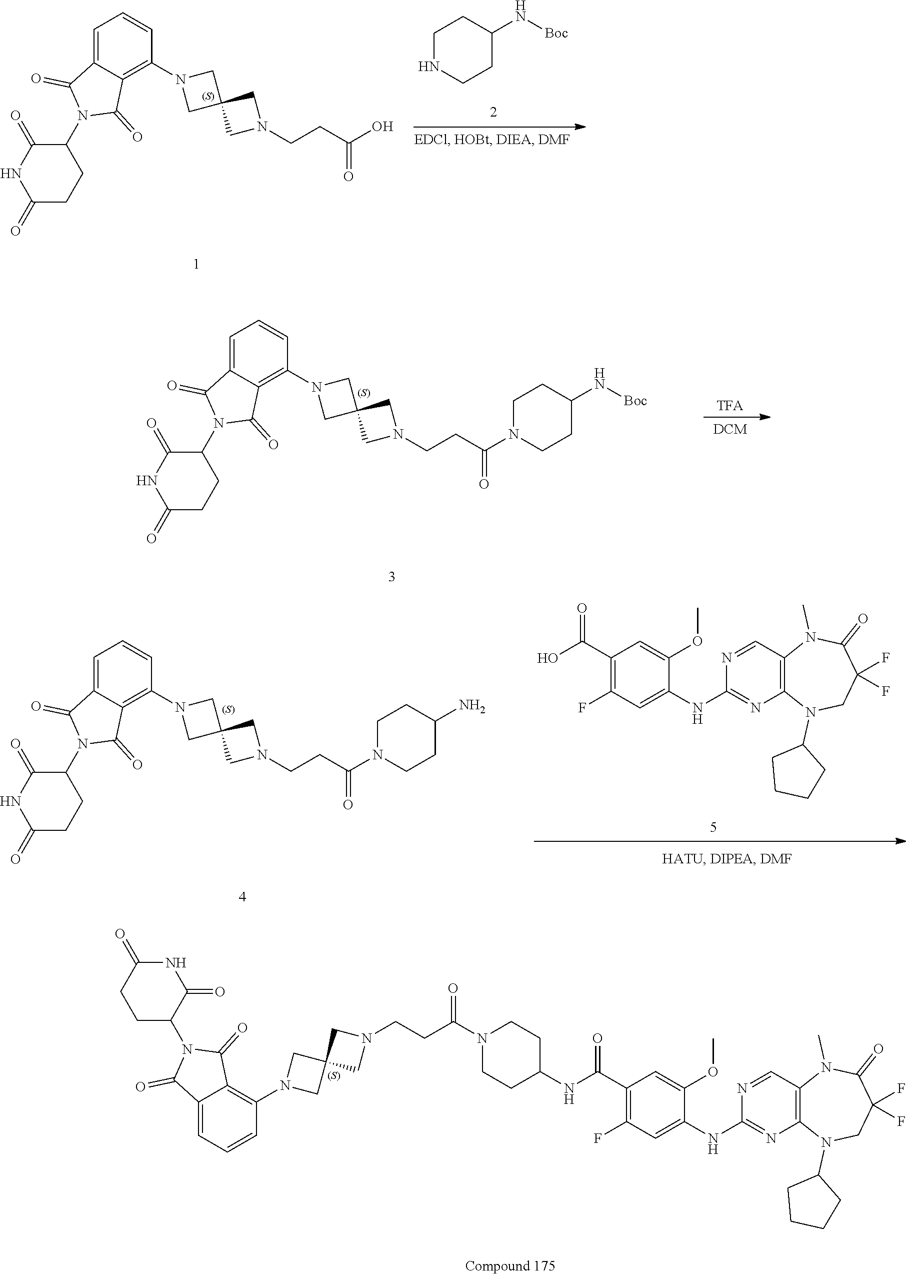

### Step 1. Synthesis of tert-butyl (1-(3-(6-(2-(2,6-dioxopiperidin-3-yl)-1,3-dioxoisoindolin-4-yl)-2,6-diazaspiro[3.3]heptan-2-yl)propanoyl)piperidin-4-yl)carbamate (3)

To a solution of 3-(6-(2-(2,6-dioxopiperidin-3-yl)-1,3-dioxoisoindolin-4-yl)-2,6-diazaspiro[3.3]heptan-2-yl)propanoic acid (1.7 g, 3.99 mmol) and tert-butyl piperidin-4-ylcarbamate (800 mg, 3.99 mmol) in DMF (15 mL) were added EDCI (1.15 g, 5.98 mmol), HOBt (1.08 g, 7.97 mmol) and DIPEA (1.55 g, 11.96 mmol, 2.08 mL) and the mixture was stirred at 20° C. for 14 h. LCMS showed 85% of the desired mass was detected and the starting material was consumed. The mixture was diluted with H₂O (30 mL) and extracted with EtOAc (10 mL×3), the combined organic layer was washed with brine (10 mL×3), dried over Na₂SO₄ and filtered. The filtrate was concentrated under reduced pressure. The residue was purified by flash silica gel chromatography (12 g SepaFlash® Silica Flash Column, Eluent of 2˜30% EtOH/EtOAc gradient @50 mL/min) to afford tert-butyl (1-(3-(6-(2-(2,6-dioxopiperidin-3-yl)-1,3-dioxoisoindolin-4-yl)-2,6-diazaspiro[3.3]heptan-2-yl)propanoyl)piperidin-4-yl)carbamate (280 mg, 460.01 μmol, 11.54% yield, 100% purity) as a yellow solid. MS(M+H)⁺=609.3

### Step 2. Synthesis of 4-(6-(3-(4-aminopiperidin-1-yl)-3-oxopropyl)-2,6-diazaspiro[3.3]heptan-2-yl)-2-(2,6-dioxopiperidin-3-yl)isoindoline-1,3-dione (4)

To a solution of tert-butyl (1-(3-(6-(2-(2,6-dioxopiperidin-3-yl)-1,3-dioxoisoindolin-4-yl)-2,6-diazaspiro[3.3]heptan-2-yl)propanoyl)piperidin-4-yl)carbamate (130 mg, 213.58 μmol) in DCM (1.3 mL) was added TFA (400.40 mg, 3.51 mmol, 260.00 μL) at −10° C. and the mixture was stirred at −10° C. for 1.5 h. LCMS showed 74% of the desired mass was detected and 19% of the starting material remained. The mixture was stirred at −5° C. for 30 min. LCMS showed 97% of the desired mass was detected and the starting material was consumed. The mixture was concentrated under reduced pressure to afford 4-(6-(3-(4-aminopiperidin-1-yl)-3-oxopropyl)-2,6-diazaspiro[3.3]heptan-2-yl)-2-(2,6-dioxopiperidin-3-yl)isoindoline-1,3-dione (160 mg, crude, 2 TFA) as yellow oil. MS(M+H)⁺=509.1

### Step 3. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(6-(2-(2,6-dioxopiperidin-3-yl)-1,3-dioxoisoindolin-4-yl)-2,6-diazaspiro[3.3]heptan-2-yl)propanoyl)piperidin-4-yl)-2-fluoro-5-methoxybenzamide (Compound 175)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (70 mg, 150.40 μmol) in DMF (1 mL) were added HATU (86 mg, 226.18 μmol) and DIPEA (29.68 mg, 229.64 μmol, 40 μL) and the mixture was stirred at 20° C. for 15 min. Then a solution of 4-(6-(3-(4-aminopiperidin-1-yl)-3-oxopropyl)-2,6-diazaspiro[3.3]heptan-2-yl)-2-(2,6-dioxopiperidin-3-yl)isoindoline-1,3-dione (160 mg, 217.21 μmol, 2 TFA) and DIPEA (192.92 mg, 1.49 mmol, 260 μL) in DMF (1 mL) was added at −15° C. and the mixture was stirred at 0° C. for 45 min. The mixture was diluted with H₂O (10 mL) and extracted with EtOAc (10 mL×3), the combined organic layer was washed with brine (10 mL×3), dried over Na₂SO₄ and filtered. The filtrate was concentrated under reduced pressure. The residue was purified by prep-TLC (Dichloromethane:Methanol=10:1) to afford the product (50 mg, 93% purity) and then purified by prep-HPLC (column: Waters Xbridge 150*25 mm*5 um; mobile phase: [water (10 mM NH₄HCO₃)-ACN]; B %: 41%-71%, min) and lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(6-(2-(2,6-dioxopiperidin-3-yl)-1,3-dioxoisoindolin-4-yl)-2,6-diazaspiro[3.3]heptan-2-yl)propanoyl)piperidin-4-yl)-2-fluoro-5-methoxybenzamide (32.7 mg, 33.52 μmol, 22.29% yield, 98% purity) as yellow solid. MS(M+H)⁺=956.3.

¹H NMR (400 MHz, DMSO-d₆) δ=11.08 (s, 1H), 8.30 (s, 1H), 8.25 (d, J=13.2 Hz, 1H), 8.05 (s, 1H), 8.01-7.97 (m, 1H), 7.59-7.54 (m, 1H), 7.20 (d, J=6.7 Hz, 1H), 7.12 (d, J=7.1 Hz, 1H), 6.78 (d, J=8.4 Hz, 1H), 5.09-5.01 (m, 1H), 4.86-4.77 (m, 1H), 4.34-4.20 (m, 5H), 4.08 (t, J=13.7 Hz, 2H), 4.03-3.96 (m, 1H), 3.92 (s, 3H), 3.88-3.80 (m, 1H), 3.30-3.24 (m, 7H), 3.18-3.07 (m, 1H), 2.94-2.81 (m, 2H), 2.77-2.69 (m, 1H), 2.62-2.53 (m, 2H), 2.35-2.28 (m, 2H), 2.04-1.93 (m, 3H), 1.91-1.76 (m, 3H), 1.76-1.69 (m, 2H), 1.68-1.56 (m, 4H), 1.52-1.31 (m, 2H).

### Example 176. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-(6-(2-(2,6-dioxopiperidin-3-yl)-1,3-dioxoisoindolin-4-yl)-2,6-diazaspiro[3.3]heptan-2-yl)butanoyl)piperidin-4-yl)-2-fluoro-5-methoxybenzamide (Compound 176)

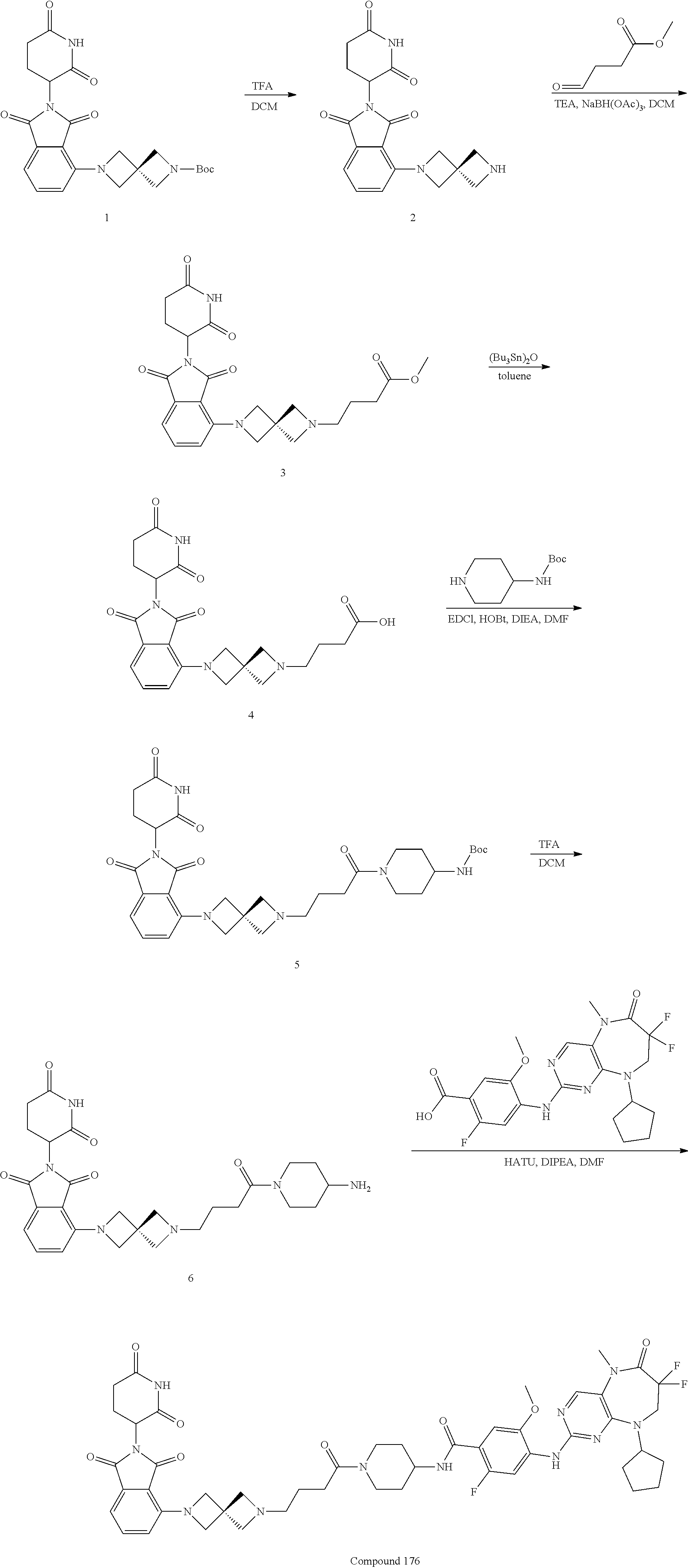

### Step 1. Synthesis of methyl 4-(6-(2-(2,6-dioxopiperidin-3-yl)-1,3-dioxoisoindolin-4-yl)-2,6-diazaspiro[3.3]heptan-2-yl)butanoate (3)

To a solution of tert-butyl 6-(2-(2,6-dioxopiperidin-3-yl)-1,3-dioxoisoindolin-4-yl)-2,6-diazaspiro[3.3]heptane-2-carboxylate (1.1 g, 2.42 mmol) in DCM (15 mL) was added TFA (2.76 g, 24.20 mmol, 1.79 mL) and the mixture was stirred at 20° C. for 5 h. LCMS showed 68% of 2-(2,6-dioxopiperidin-3-yl)-4-(2,6-diazaspiro[3.3]heptan-2-yl)isoindoline-1,3-dione mass was detected. TEA (2.45 g, 24.20 mmol, 3.37 mL) was added at 0° C. and then a solution of methyl 4-oxobutanoate (843 mg, 7.26 mmol) in DCM (5 mL) was added and the mixture was stirred at 0° C. for 15 min. NaBH(OAc)₃ (1.54 g, 7.26 mmol) was added and the mixture was stirred at 20° C. for 14 h. LCMS showed 30% of the desired mass was detected. The mixture was diluted with H₂O (30 mL) and extracted with EtOAc (20 mL×3), the combined organic layer was washed with H₂O (20 mL), dried over Na₂SO₄ and filtered. The filtrate was concentrated under reduced pressure. The residue was purified by flash silica gel chromatography (12 g SepaFlash® Silica Flash Column, Eluent of 0˜30% MeOH/EtOAc gradient @50 mL/min) to afford methyl 4-(6-(2-(2,6-dioxopiperidin-3-yl)-1,3-dioxoisoindolin-4-yl)-2,6-diazaspiro[3.3]heptan-2-yl)butanoate (420 mg, 776.28 μmol, 32.07% yield, 84% purity) as yellow solid. MS(M+H)⁺=455.1

### Step 2. Synthesis of 4-(6-(2-(2,6-dioxopiperidin-3-yl)-1,3-dioxoisoindolin-4-yl)-2,6-diazaspiro[3.3]heptan-2-yl)butanoic acid (4)

To a solution of methyl 4-(6-(2-(2,6-dioxopiperidin-3-yl)-1,3-dioxoisoindolin-4-yl)-2,6-diazaspiro[3.3]heptan-2-yl)butanoate (420 mg, 776.28 μmol, 84% purity) in toluene (10 mL) was added (Bu₃Sn)₂O (2.25 g, 3.77 mmol, 1.92 mL) and the mixture was stirred at 110° C. for 28 h. LCMS showed the desired mass was detected and trace of the starting material remained after work up. The mixture was concentrated under reduced pressure to afford 4-(6-(2-(2,6-dioxopiperidin-3-yl)-1,3-dioxoisoindolin-4-yl)-2,6-diazaspiro[3.3]heptan-2-yl)butanoic acid (2.5 g, crude) as yellow oil. MS(M+H)⁺=441.5

### Step 3. Synthesis of tert-butyl (1-(4-(6-(2-(2,6-dioxopiperidin-3-yl)-1,3-dioxoisoindolin-4-yl)-2,6-diazaspiro[3.3]heptan-2-yl)butanoyl)piperidin-4-yl)carbamate (5)

To a solution of tert-butyl piperidin-4-ylcarbamate (1.14 g, 5.68 mmol) and 4-(6-(2-(2,6-dioxopiperidin-3-yl)-1,3-dioxoisoindolin-4-yl)-2,6-diazaspiro[3.3]heptan-2-yl)butanoic acid (2.5 g, crude) in DMF (10 mL) were added DIPEA (2.20 g, 17.04 mmol, 2.97 mL), EDCI (1.63 g, 8.51 mmol) and HOBt (1.53 g, 11.35 mmol) and the mixture was stirred at 20° C. for 14 h. LCMS showed 60% of the desired mass was detected and the starting material was consumed. The mixture was diluted with H₂O (10 mL) and extracted with EtOAc (10 mL×3), the combined organic layer was washed with brine (10 mL×3), dried over Na₂SO₄ and filtered. The filtrate was concentrated under reduced pressure. The residue was purified by flash silica gel chromatography (12 g SepaFlash® Silica Flash Column, Eluent of 2˜10% MeOH/EtOAc gradient @50 mL/min) to afford tert-butyl (1-(4-(6-(2-(2,6-dioxopiperidin-3-yl)-1,3-dioxoisoindolin-4-yl)-2,6-diazaspiro[3.3]heptan-2-yl)butanoyl)piperidin-4-yl)carbamate (330 mg, 514.04 umol, 9.06% yield, 97% purity) as yellow solid. MS(M+H)⁺=623.0

### Step 4. Synthesis of 4-(6-(4-(4-aminopiperidin-1-yl)-4-oxobutyl)-2,6-diazaspiro[3.3]heptan-2-yl)-2-(2,6-dioxopiperidin-3-yl)isoindoline-1,3-dione (6)

To a solution of tert-butyl (1-(4-(6-(2-(2,6-dioxopiperidin-3-yl)-1,3-dioxoisoindolin-4-yl)-2,6-diazaspiro[3.3]heptan-2-yl)butanoyl)piperidin-4-yl)carbamate (100 mg, 160.59 μmol) in DCM (1 mL) was added TFA (308.00 mg, 2.70 mmol, 0.2 mL) at −10° C. and the mixture was stirred at −10° C. for 1 h. LCMS showed 66% of the desired mass was detected and 18% of the starting material remained. LCMS showed 92% of the desired mass was detected and 1% of the starting material remained. The mixture was concentrated under reduced pressure to afford 4-(6-(4-(4-aminopiperidin-1-yl)-4-oxobutyl)-2,6-diazaspiro[3.3]heptan-2-yl)-2-(2,6-dioxopiperidin-3-yl)isoindoline-1,3-dione (130 mg, crude, 2 TFA) as yellow oil. MS(M+H)⁺=523.1.

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-(6-(2-(2,6-dioxopiperidin-3-yl)-1,3-dioxoisoindolin-4-yl)-2,6-diazaspiro[3.3]heptan-2-yl)butanoyl)piperidin-4-yl)-2-fluoro-5-methoxybenzamide (Compound 176)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (60 mg, 128.91 μmol) and HATU (73.71 mg, 193.87 μmol) in DMF (1 mL) was added DIPEA (29.68 mg, 229.64 μmol, 40 μL) and the mixture was stirred at 20° C. for 15 min. Then a solution of 4-(6-(4-(4-aminopiperidin-1-yl)-4-oxobutyl)-2,6-diazaspiro[3.3]heptan-2-yl)-2-(2,6-dioxopiperidin-3-yl)isoindoline-1,3-dione (130.00 mg, 173.19 μmol, 2 TFA) and DIPEA (103.88 mg, 803.76 μmol, 140 μL) in DMF (1 mL) was added at −5° C. and the mixture was stirred at 0° C. for 45 min. LCMS showed 70% of the desired mass was detected. The mixture was diluted with H₂O (10 mL) and extracted with EtOAc (10 mL×3), the combined organic layer was washed with H₂O (10 mL×3), dried over Na₂SO₄ and filtered. The filtrate was concentrated under reduced pressure. The crude was purified by prep-HPLC (column: Waters Xbridge 150*25 mm*5 um; mobile phase: [water (10 mM NH₄HCO₃)-ACN]; B %: 41%-71%, min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-(6-(2-(2,6-dioxopiperidin-3-yl)-1,3-dioxoisoindolin-4-yl)-2,6-diazaspiro[3.3]heptan-2-yl)butanoyl)piperidin-4-yl)-2-fluoro-5-methoxybenzamide (35 mg, 89% purity). Then the product was purified by prep-TLC (Dichloromethane:Methanol=10/1) to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(4-(6-(2-(2,6-dioxopiperidin-3-yl)-1,3-dioxoisoindolin-4-yl)-2,6-diazaspiro[3.3]heptan-2-yl)butanoyl)piperidin-4-yl)-2-fluoro-5-methoxybenzamide (13.7 mg, 13.84 μmol, 10.74% yield, 98% purity) as yellow solid. MS(M+H)⁺=970.3.

¹H NMR (400 MHz, DMSO-d₆) δ=11.06 (s, 1H), 8.30 (s, 1H), 8.25 (d, J=13.3 Hz, 1H), 8.03 (s, 1H), 7.98-7.92 (m, 1H), 7.58-7.53 (m, 1H), 7.20 (d, J=6.7 Hz, 1H), 7.11 (d, J=7.2 Hz, 1H), 6.78 (d, J=8.7 Hz, 1H), 5.08-5.01 (m, 1H), 4.85-4.78 (m, 1H), 4.33-4.22 (m, 5H), 4.13-3.98 (m, 3H), 3.93-3.81 (m, 4H), 3.31-3.29 (m, 4H), 3.26-3.22 (m, 3H), 3.17-3.08 (m, 1H), 2.93-2.81 (m, 1H), 2.76-2.65 (m, 2H), 2.61-2.55 (m, 2H), 2.39-2.27 (m, 4H), 2.03-1.92 (m, 3H), 1.90-1.77 (m, 2H), 1.75-1.69 (m, 2H), 1.68-1.55 (m, 4H), 1.52-1.43 (m, 3H).

### Example 177. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((3S)-1-(3-(6-(2-(2,6-dioxopiperidin-3-yl)-1,3-dioxoisoindolin-4-yl)-2,6-diazaspiro[3.3]heptan-2-yl)propanoyl)pyrrolidin-3-yl)-2-fluoro-5-methoxybenzamide (Compound 177)

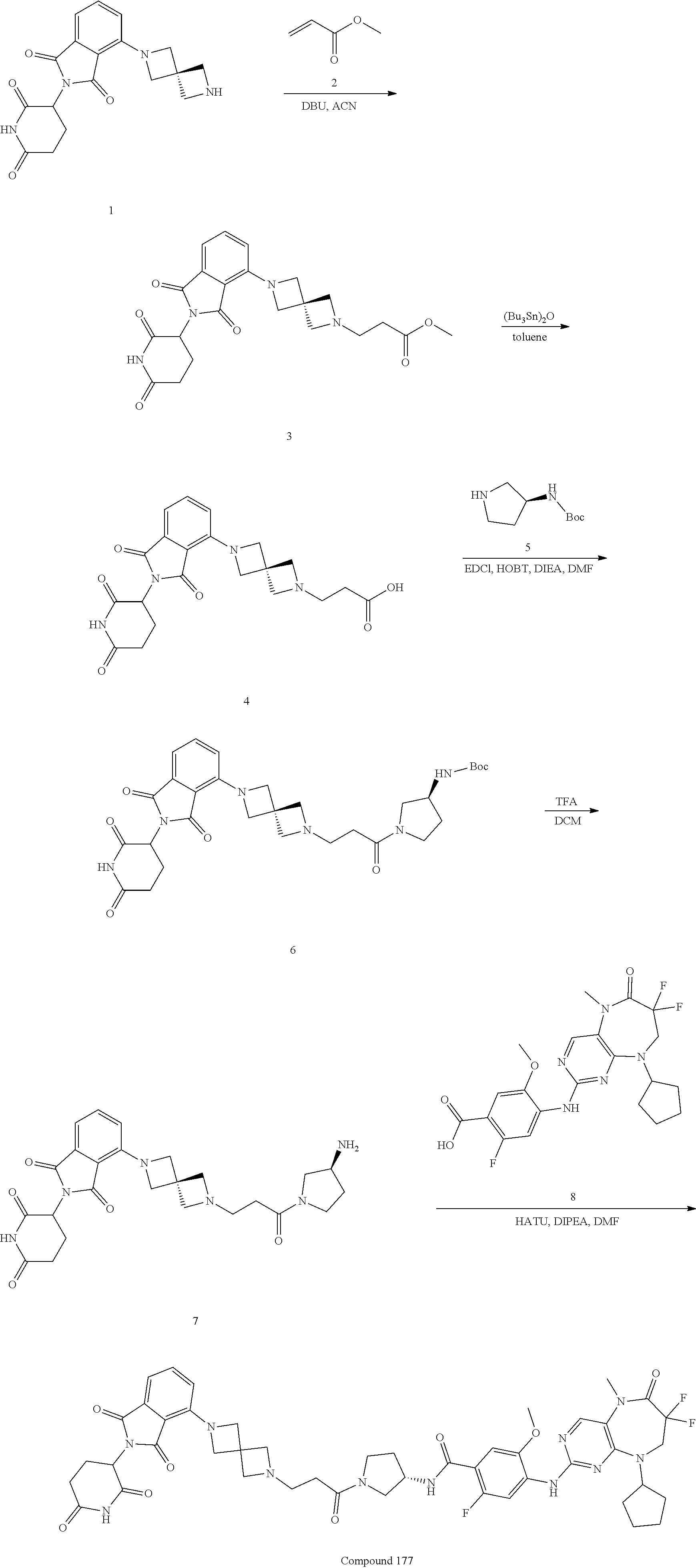

### Step 1. Synthesis of methyl 3-(6-(2-(2,6-dioxopiperidin-3-yl)-1,3-dioxoisoindolin-4-yl)-2,6-diazaspiro[3.3]heptan-2-yl)propanoate (3)

To a solution of 2-(2,6-dioxopiperidin-3-yl)-4-(2,6-diazaspiro[3.3]heptan-2-yl)isoindoline-1,3-dione (1.7 g, 3.63 mmol, TFA) and methyl acrylate (5.22 g, 60.63 mmol, 5.46 mL) in ACN (30 mL) was added DBU (11.11 g, 72.98 mmol, 11 mL) and the mixture was stirred at −30° C. for 1 h. LCMS showed the starting material was consumed and 68% of the desired mass was detected. The mixture was diluted with H₂O (20 mL) at −5° C. and extracted with EtOAc (20 mL×3), the combined organic layer was washed with brine (20 mL×3), dried over Na₂SO₄ and filtered. The filtrate was concentrated under reduced pressure. The residue was purified by flash silica gel chromatography (12 g SepaFlash® Silica Flash Column, Eluent of 0˜30% MeOH/EtOAc gradient @50 mL/min) to afford methyl 3-(6-(2-(2,6-dioxopiperidin-3-yl)-1,3-dioxoisoindolin-4-yl)-2,6-diazaspiro[3.3]heptan-2-yl)propanoate (850 mg, 1.79 mmol, 49.45% yield, 93% purity) as a yellow solid. MS(M+H)⁺=441.1

### Step 2. Synthesis of 3-(6-(2-(2,6-dioxopiperidin-3-yl)-1,3-dioxoisoindolin-4-yl)-2,6-diazaspiro[3.3]heptan-2-yl)propanoic acid (4)

To a solution of methyl 3-(6-(2-(2,6-dioxopiperidin-3-yl)-1,3-dioxoisoindolin-4-yl)-2,6-diazaspiro[3.3]heptan-2-yl)propanoate (850 mg, 1.93 mmol) in toluene (20 mL) was added (Bu₃Sn)₂O (2.81 g, 4.71 mmol, 2.40 mL) and the mixture was stirred at 110° C. for 28 h. LCMS showed 85% of the desired mass was detected. The mixture was concentrated under reduced pressure to afford 3-(6-(2-(2,6-dioxopiperidin-3-yl)-1,3-dioxoisoindolin-4-yl)-2,6-diazaspiro[3.3]heptan-2-yl)propanoic acid (3.4 g, crude) as a yellow oil. MS(M+H)⁺=427.1

### Step 3. Synthesis of tert-butyl ((3S)-1-(3-(6-(2-(2,6-dioxopiperidin-3-yl)-1,3-dioxoisoindolin-4-yl)-2,6-diazaspiro[3.3]heptan-2-yl)propanoyl)pyrrolidin-3-yl)carbamate (6)

To a solution of 3-(6-(2-(2,6-dioxopiperidin-3-yl)-1,3-dioxoisoindolin-4-yl)-2,6-diazaspiro[3.3]heptan-2-yl)propanoic acid (1.7 g, 3.99 mmol) and (S)-tert-butyl pyrrolidin-3-ylcarbamate (750 mg, 4.03 mmol) in DMF (15 mL) were added EDCI (1.15 g, 5.98 mmol), HOBt (1.08 g, 7.97 mmol) and DIPEA (1.55 g, 11.96 mmol, 2.08 mL) and the mixture was stirred at 20° C. for 14 h. LCMS showed 89% of the desired mass was detected and the starting material was consumed. The mixture was diluted with H₂O (30 mL) and extracted with EtOAc (10 mL×3), the combined organic layer was washed with brine (10 mL×3), dried over Na₂SO₄ and filtered. The filtrate was concentrated under reduced pressure. The residue was purified by flash silica gel chromatography (12 g SepaFlash® Silica Flash Column, Eluent of 0˜30% EtOH/EtOAc gradient @50 mL/min) to afford tert-butyl ((3S)-1-(3-(6-(2-(2,6-dioxopiperidin-3-yl)-1,3-dioxoisoindolin-4-yl)-2,6-diazaspiro[3.3]heptan-2-yl)propanoyl)pyrrolidin-3-yl)carbamate (350 mg, 553.26 μmol, 13.88% yield, 94% purity) as a yellow solid. MS(M+H)⁺=595.1

### Step 4. Synthesis of 4-(6-(3-((S)-3-aminopyrrolidin-1-yl)-3-oxopropyl)-2,6-diazaspiro[3.3]heptan-2-yl)-2-(2,6-dioxopiperidin-3-yl)isoindoline-1,3-dione (7)

To a solution of tert-butyl ((3S)-1-(3-(6-(2-(2,6-dioxopiperidin-3-yl)-1,3-dioxoisoindolin-4-yl)-2,6-diazaspiro[3.3]heptan-2-yl)propanoyl)pyrrolidin-3-yl)carbamate (130 mg, 218.61 μmol) in DCM (1.3 mL) was added TFA (400.40 mg, 3.51 mmol, 260.00 μL) at 0° C. and the mixture was stirred at 0° C. for 3 h. LCMS showed 79% of the desired mass was detected. The reaction mixture was concentrated under reduced pressure to afford 4-(6-(3-((S)-3-aminopyrrolidin-1-yl)-3-oxopropyl)-2,6-diazaspiro[3.3]heptan-2-yl)-2-(2,6-dioxopiperidin-3-yl)isoindoline-1,3-dione (160 mg, crude) as a yellow oil. MS(M+H)⁺=495.1

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((3S)-1-(3-(6-(2-(2,6-dioxopiperidin-3-yl)-1,3-dioxoisoindolin-4-yl)-2,6-diazaspiro[3.3]heptan-2-yl)propanoyl)pyrrolidin-3-yl)-2-fluoro-5-methoxybenzamide (Compound 177)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (80 mg, 171.89 μmol) in DMF (1.5 mL) were added HATU (98 mg, 257.74 μmol) and DIPEA (59.36 mg, 459.29 μmol, 80.00 μL) and the mixture was stirred at 20° C. for 15 min. Then a solution of 4-(6-(3-((S)-3-aminopyrrolidin-1-yl)-3-oxopropyl)-2,6-diazaspiro[3.3]heptan-2-yl)-2-(2,6-dioxopiperidin-3-yl)isoindoline-1,3-dione (160 mg, 221.43 μmol, 2 TFA) and DIPEA (237.44 mg, 1.84 mmol, 320.00 μL) in DMF (1.5 mL) was added at 0° C. and the mixture was stirred at 20° C. for 45 min. LCMS showed the desired mass was detected. The mixture was diluted with H₂O (10 mL) and extracted with EtOAc (10 mL×3), the combined organic layer was washed with brine (10 mL×3), dried over Na₂SO₄ and filtered. The filtrate was concentrated under reduced pressure. The residue was purified by prep-TLC (Dichloromethane:Methanol=10:1) and then purified by prep-HPLC (column: Waters Xbridge 150*25 mm*5 um; mobile phase: [water (10 mM NH₄HCO₃)-ACN]; B %: 37%-67%, min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-((3S)-1-(3-(6-(2-(2,6-dioxopiperidin-3-yl)-1,3-dioxoisoindolin-4-yl)-2,6-diazaspiro[3.3]heptan-2-yl)propanoyl)pyrrolidin-3-yl)-2-fluoro-5-methoxybenzamide (37.9 mg, 39.43 μmol, 22.94% yield, 98% purity) as a yellow solid. MS(M+H)⁺=942.3.

¹H NMR (400 MHz, DMSO-d₆) δ=11.06 (s, 1H), 8.30-8.20 (m, 3H), 8.05-7.98 (m, 1H), 7.57-7.48 (m, 1H), 7.23-7.17 (m, 1H), 7.12-7.04 (m, 1H), 6.78-6.67 (m, 1H), 5.08-4.99 (m, 1H), 4.86-4.74 (m, 1H), 4.50-4.35 (m, 1H), 4.20 (br d, J=15.4 Hz, 4H), 4.12-4.02 (m, 2H), 3.91 (s, 3H), 3.78-3.70 (m, 1H), 3.62-3.40 (m, 4H), 3.29-3.21 (m, 7H), 2.94-2.81 (m, 1H), 2.62-2.53 (m, 4H), 2.27-2.16 (m, 2H), 2.03-1.90 (m, 4H), 1.77-1.54 (m, 6H).

### Example 178. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((3S)-1-(5-(6-(2-(2,6-dioxopiperidin-3-yl)-1,3-dioxoisoindolin-4-yl)-2,6-diazaspiro[3.3]heptan-2-yl)pentanoyl)pyrrolidin-3-yl)-2-fluoro-5-methoxybenzamide (Compound 178)

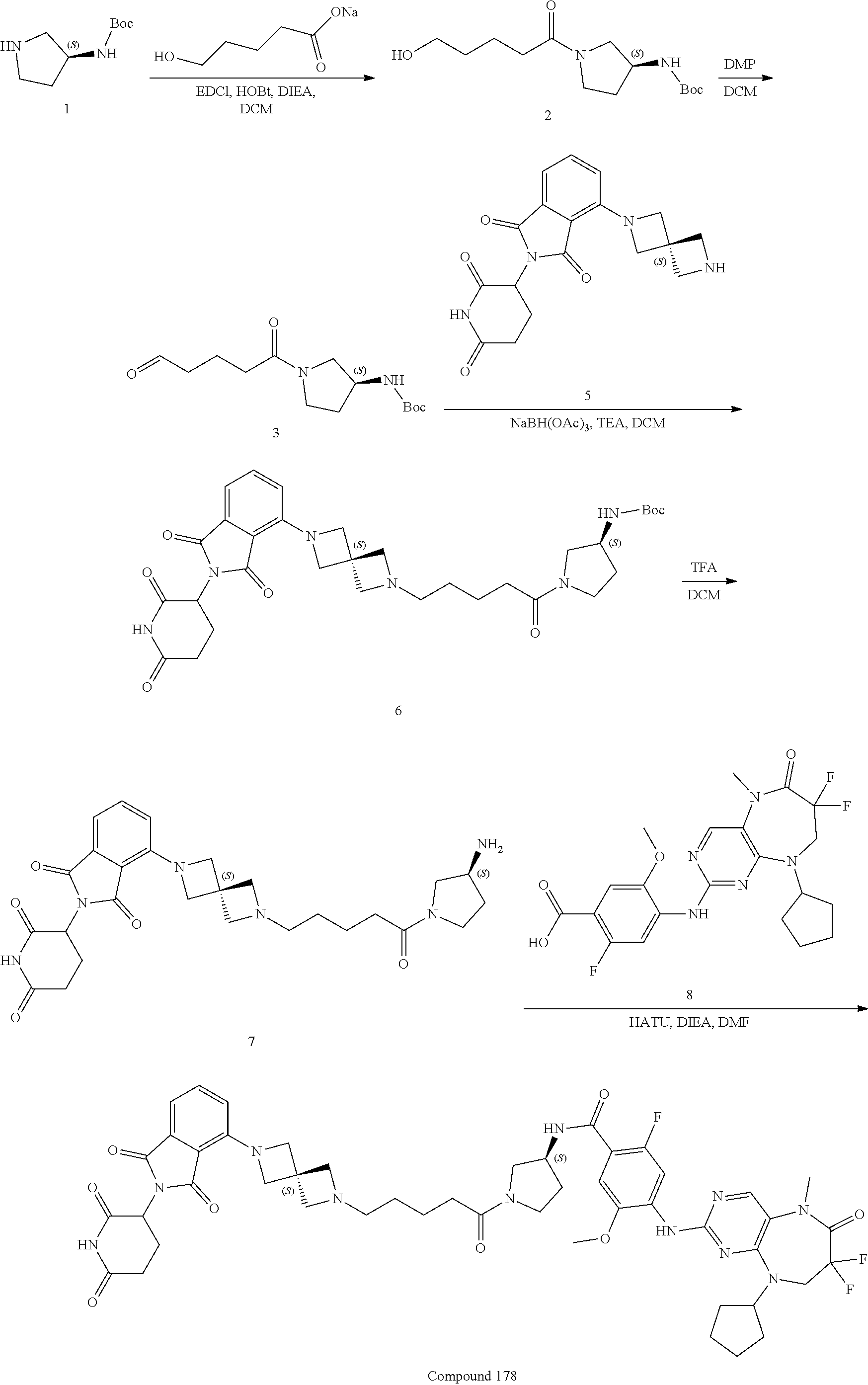

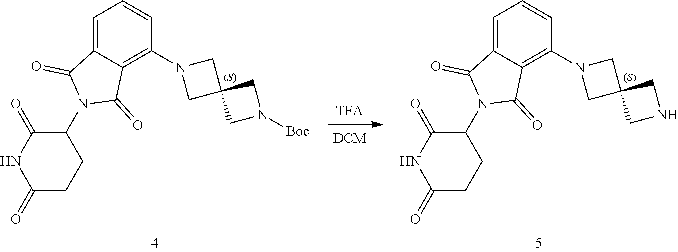

The compound 178 was synthesized by the method described in the scheme similar to the method described in Example 177.

MS(M+H)⁺=970.3, ¹H NMR (400 MHz, DMSO-d₆) δ=11.08-11.04 (m, 1H), 8.30-8.22 (m, 3H), 8.03 (s, 1H), 7.58-7.51 (m, 1H), 7.19 (d, J=6.6 Hz, 1H), 7.13-7.08 (m, 1H), 6.75 (t, J=8.5 Hz, 1H), 5.07-5.00 (m, 1H), 4.86-4.78 (m, 1H), 4.51-4.33 (m, 1H), 4.28-4.19 (m, 4H), 4.07 (t, J=14.0 Hz, 2H), 3.91 (s, 3H), 3.77-3.70 (m, 1H), 3.61-3.53 (m, 2H), 3.51-3.40 (m, 8H), 2.93-2.81 (m, 1H), 2.62-2.55 (m, 2H), 2.40-2.29 (m, 2H), 2.26-2.17 (m, 2H), 2.03-1.90 (m, 4H), 1.76-1.56 (m, 7H), 1.53-1.44 (m, 2H), 1.34-1.22 (m, 2H).

### Example 179. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(5-(2-(2,6-dioxopiperidin-3-yl)-1,3-dioxoisoindolin-4-yl)pent-4-ynoyl)piperidin-4-yl)-3-methoxybenzamide (Compound 179)

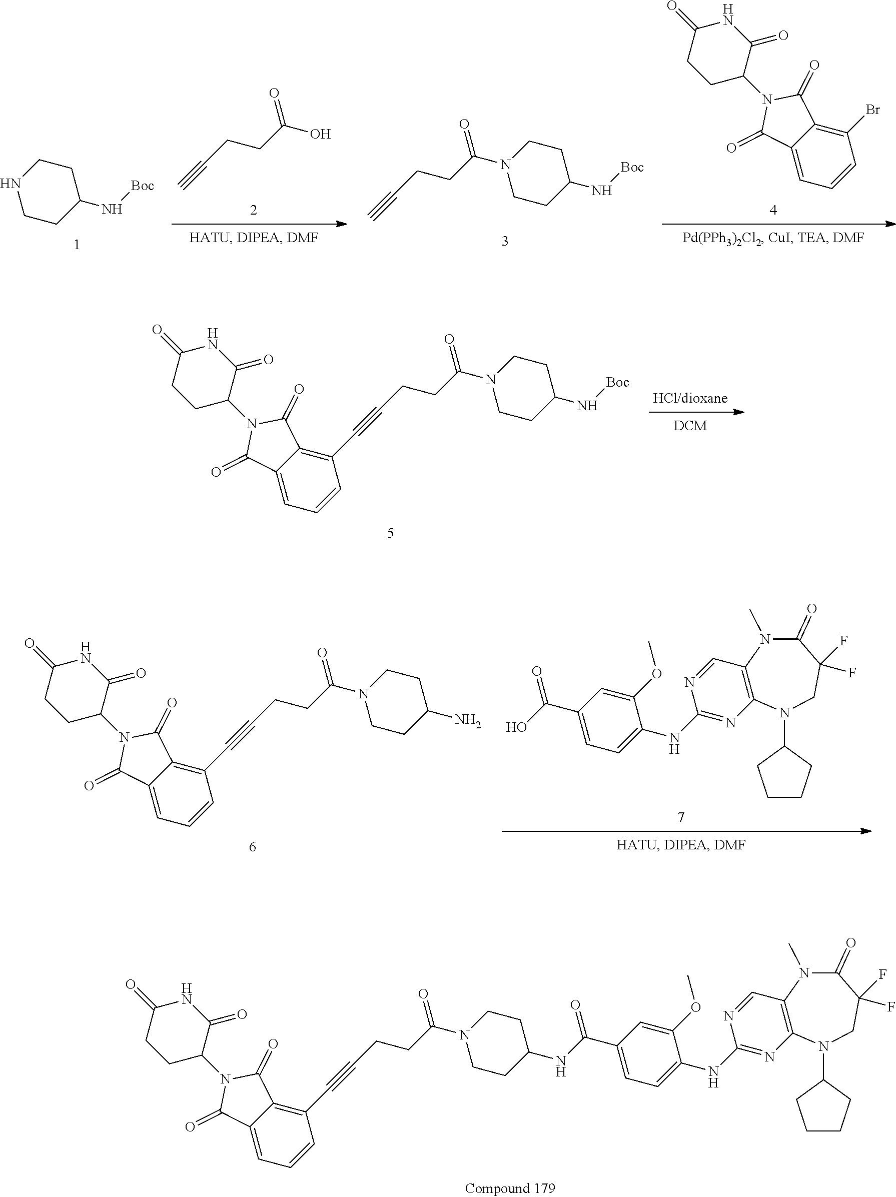

### Step 1. Synthesis of tert-butyl (1-(pent-4-ynoyl)piperidin-4-yl)carbamate (3)

To a solution of tert-butyl piperidin-4-ylcarbamate (500 mg, 2.50 mmol) and pent-4-ynoic acid (244.91 mg, 2.50 mmol) in DMF (7 mL) were added HATU (1.42 g, 3.74 mmol) and DIEA (967.98 mg, 7.49 mmol, 1.30 mL). The mixture was stirred at 25° C. for 2 h. TLC (Petroleum ether:EtOAc=1:1) indicated tert-butyl piperidin-4-ylcarbamate was consumed completely and one new spot was formed. The reaction mixture was diluted with water (100 mL) and extracted with EtOAc (30 mL×3). The combined organic layers were washed with brine (30 mL), dried over anhydrous sodium sulfate, filtered and concentrated under reduced pressure. The residue was purified by column chromatography (SiO₂, Petroleum ether/EtOAc=1/0 to 1/1) to afford tert-butyl (1-(pent-4-ynoyl)piperidin-4-yl)carbamate (520 mg, 1.85 mmol, 74.29% yield) as a white solid. MS(M+H)⁺=281.4

### Step 2. Synthesis of tert-butyl (1-(5-(2-(2,6-dioxopiperidin-3-yl)-1,3-dioxoisoindolin-4-yl)pent-4-ynoyl)piperidin-4-yl)carbamate (5)

To a solution of tert-butyl (1-(pent-4-ynoyl)piperidin-4-yl)carbamate (250 mg, 891.71 umol) and 4-bromo-2-(2,6-dioxopiperidin-3-yl)isoindoline-1,3-dione (300.62 mg, 891.71 umol) in DMF (5 mL) were added Pd(PPh₃)₂Cl₂ (62.59 mg, 89.17 umol), CuI (33.97 mg, 178.34 umol) and TEA (541.38 mg, 5.35 mmol, 744.68 uL). The mixture was stirred at 70° C. for 0.5 h under N₂ atmosphere. LCMS showed tert-butyl (1-(pent-4-ynoyl)piperidin-4-yl)carbamate was consumed completely, ˜40% desired mass was detected. The reaction mixture was diluted with water (50 mL) and extracted with EtOAc (20 mL×3). The combined organic layers were washed with brine (20 mL), dried over anhydrous sodium sulfate, filtered and concentrated under reduced pressure. The residue was purified by column chromatography (SiO₂, Petroleum ether/EtOAc=1/0 to 1/1) to afford tert-butyl (1-(5-(2-(2,6-dioxopiperidin-3-yl)-1,3-dioxoisoindolin-4-yl)pent-4-ynoyl)piperidin-4-yl)carbamate (360 mg, 530.03 umol, 59.44% yield, 79% purity) as a light yellow solid. MS(M+H)⁺=537.1

### Step 3. Synthesis of 4-(5-(4-aminopiperidin-1-yl)-5-oxopent-1-yn-1-yl)-2-(2,6-dioxopiperidin-3-yl)isoindoline-1,3-dione (6)

A mixture of tert-butyl (1-(5-(2-(2,6-dioxopiperidin-3-yl)-1,3-dioxoisoindolin-4-yl)pent-4-ynoyl)piperidin-4-yl)carbamate (100 mg, 186.37 umol) and HCl/dioxane (4 M, 1.5 mL) in DCM (1.5 mL) was stirred at 25° C. for 0.5 h. LCMS showed the starting material was consumed completely and one main peak with desired mass was detected. The reaction mixture was concentrated under reduced pressure to afford 4-(5-(4-aminopiperidin-1-yl)-5-oxopent-1-yn-1-yl)-2-(2,6-dioxopiperidin-3-yl)isoindoline-1,3-dione (100 mg, crude, HCl salt) as a light yellow solid. MS(M+H)⁺=437.0

### Step 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(5-(2-(2,6-dioxopiperidin-3-yl)-1,3-dioxoisoindolin-4-yl)pent-4-ynoyl)piperidin-4-yl)-3-methoxybenzamide (Compound 179)

To a solution of 4-(5-(4-aminopiperidin-1-yl)-5-oxopent-1-yn-1-yl)-2-(2,6-dioxopiperidin-3-yl)isoindoline-1,3-dione (100 mg, 211.45 umol, HCl salt) and 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (94.61 mg, 211.45 umol) in DMF (4 mL) were added HATU (120.60 mg, 317.18 umol) and DIEA (81.99 mg, 634.36 umol, 110.49 uL). The mixture was stirred at 25° C. for 5 h. LCMS showed 4-(5-(4-aminopiperidin-1-yl)-5-oxopent-1-yn-1-yl)-2-(2,6-dioxopiperidin-3-yl)isoindoline-1,3-dione was consumed completely, ˜57% desired mass was detected. The reaction mixture was diluted with water (50 mL) and extracted with EtOAc (20 mL×3). The combined organic layers were washed with brine (20 mL), dried over anhydrous sodium sulfate, filtered and concentrated under reduced pressure. The residue was purified by prep-HPLC (column: Phenomenex luna C18 150×25 mm×10 um; mobile phase: [water(FA)-ACN]; B %: 42%-42%, 10 min) followed by prep-TLC (SiO₂, DCM:MeOH=20:1) to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(5-(2-(2,6-dioxopiperidin-3-yl)-1,3-dioxoisoindolin-4-yl)pent-4-ynoyl)piperidin-4-yl)-3-methoxybenzamide (44.9 mg, 51.34 umol, 24.28% yield, 99% purity) as a white solid. MS(M+H)⁺=866.5.

¹H NMR (400 MHz, DMSO-d₆) δ=11.15 (s, 1H), 8.32-8.24 (m, 2H), 8.20-8.13 (m, 1H), 7.97 (s, 1H), 7.90-7.78 (m, 3H), 7.54-7.44 (m, 2H), 5.19-5.09 (m, 1H), 4.83-4.72 (m, 1H), 4.45-4.35 (m, 1H), 4.11-4.00 (m, 3H), 3.98-3.95 (m, 1H), 3.94 (s, 3H), 3.33 (s, 3H), 3.20-3.12 (m, 1H), 2.95-2.84 (m, 1H), 2.75 (s, 5H), 2.64-2.57 (m, 2H), 2.11-2.03 (m, 1H), 1.99-1.81 (m, 4H), 1.77-1.68 (m, 2H), 1.66-1.55 (m, 4H), 1.53-1.38 (m, 2H).

### Example 180. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(6-(2-(2,6-dioxopiperidin-3-yl)-1,3-dioxoisoindolin-4-yl)hex-5-ynoyl)piperidin-4-yl)-3-methoxybenzamide (Compound 180)

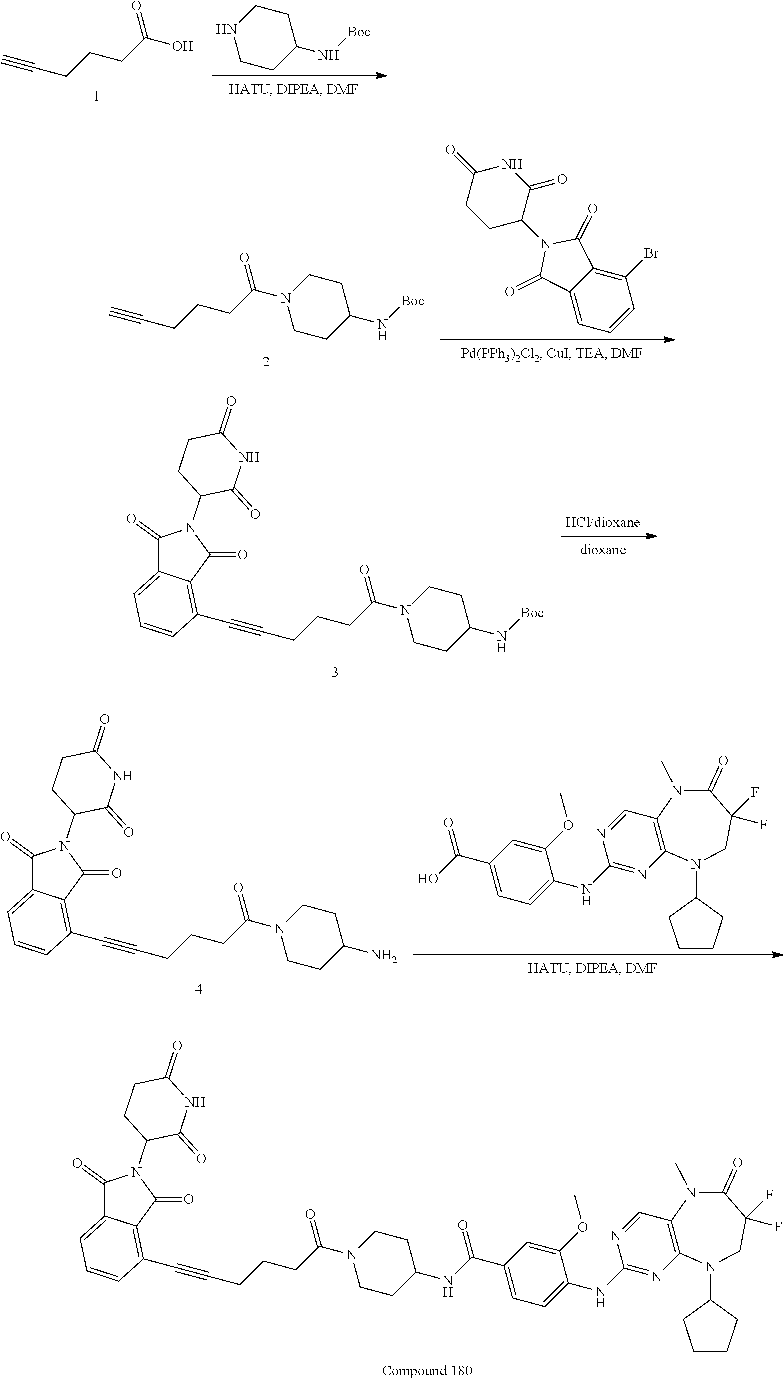

The compound 180 was synthesized by the method described in the scheme similar to the method described in Example 179.

MS(M+H)⁺=880.0, ¹H NMR (400 MHz, DMSO-d₆) δ=11.14 (s, 1H), 8.32-8.24 (m, 2H), 8.12 (dd, J=3.1, 7.1 Hz, 1H), 7.97 (s, 1H), 7.91-7.81 (m, 3H), 7.53-7.46 (m, 2H), 5.14 (dd, J=5.4, 12.6 Hz, 1H), 4.83-4.71 (m, 1H), 4.42 (d, J=11.9 Hz, 1H), 4.12-3.90 (m, 7H), 3.35 (s, 3H), 3.14 (t, J=11.6 Hz, 1H), 2.94-2.80 (m, 1H), 2.73-2.58 (m, 7H), 2.08-1.80 (m, 7H), 1.73-1.40 (m, 8H).

### Example 181. Synthesis of 4-((1′-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)-[1,4′-bipiperidin]-4-yl)ethynyl)-2-(2,6-dioxopiperidin-3-yl)isoindoline-1,3-dione (Compound 181)

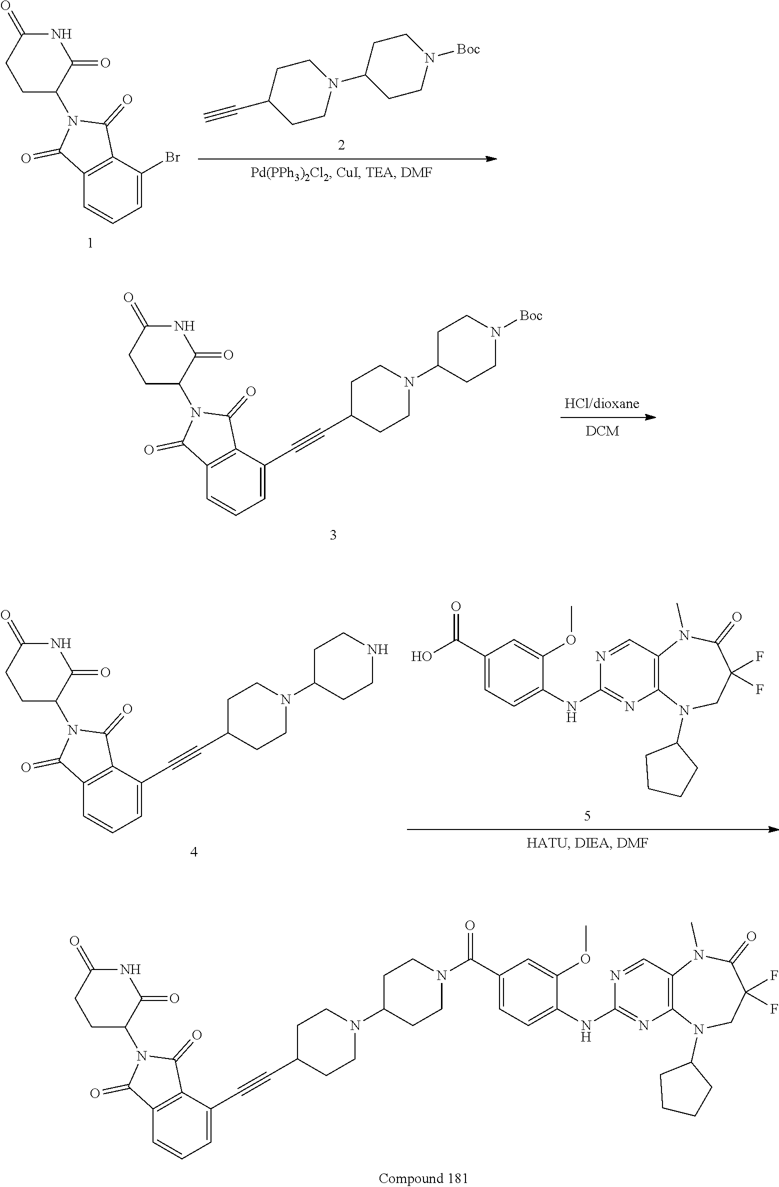 [SMILES: O=C1CCC(N2C(=O)c3cccc(Br)c3C2=O)C(=O)N1 ; C#CC1CCN(C2CCN(C(=O)OC(C)(C)C)CC2)CC1 ; CC(C)(C)OC(=O)N1CCC(N2CCC(C#Cc3cccc4c3C(=O)N(C3CCC(=O)NC3=O)C4=O)CC2)CC1 ; O=C1CCC(N2C(=O)c3cccc(C#CC4CCN(C5CCNCC5)CC4)c3C2=O)C(=O)N1 ; COc1cc(C(=O)O)ccc1Nc1ncc2c(n1)N(C1CCCC1)CC(F)(F)C(=O)N2C ; COc1cc(C(=O)N2CCC(N3CCC(C#Cc4cccc5c4C(=O)N(C4CCC(=O)NC4=O)C5=O)CC3)CC2)ccc1Nc1ncc2c(n1)N(C1CCCC1)CC(F)(F)C(=O)N2C]

### Step 1. Synthesis of tert-butyl 4-((2-(2,6-dioxopiperidin-3-yl)-1,3-dioxoisoindolin-4-yl)ethynyl)-[1,4′-bipiperidine]-1′-carboxylate (3)

To a solution of tert-butyl 4-ethynyl-[1,4′-bipiperidine]-1′-carboxylate (100 mg, 341.98 umol) and 4-bromo-2-(2,6-dioxopiperidin-3-yl)isoindoline-1,3-dione (115.29 mg, 341.98 umol) in DMF (3 mL) were added Pd(PPh₃)₂Cl₂ (24.00 mg, 34.20 umol), CuI (13.03 mg, 68.40 umol) and TEA (103.81 mg, 1.03 mmol, 142.80 uL). The mixture was stirred at 50° C. for 0.5 h. LCMS showed tert-butyl 4-ethynyl-[1,4′-bipiperidine]-1′-carboxylate was consumed completely, a peak (25%) with desired mass. The reaction mixture was diluted with water (50 mL) and extracted with EtOAc (20 mL×3). The combined organic layers were washed with brine (20 mL), dried over anhydrous sodium sulfate, filtered and concentrated under reduced pressure. The residue was purified by prep-TLC (SiO₂, EtOAc:MeOH=5:1) to afford tert-butyl 4-((2-(2,6-dioxo piperidin-3-yl)-1,3-dioxoisoindolin-4-yl)ethynyl)-[1,4′-bipiperidine]-1′-carboxylate (80 mg, 113.74 umol, 33.26% yield, 78% purity) as a light yellow solid. MS(M+H)⁺=549.3

### Step 2. Synthesis of 4-([1,4′-bipiperidin]-4-ylethynyl)-2-(2,6-dioxopiperidin-3-yl)isoindoline-1,3-dione (4)

A mixture of tert-butyl 4-((2-(2,6-dioxopiperidin-3-yl)-1,3-dioxoisoindolin-4-yl)ethynyl)-[1,4′-bipiperidine]-1′-carboxylate (80 mg, 145.82 μmol) and HCl/dioxane (4 M, 1 mL) in DCM (1 mL) was stirred at 25° C. for 0.5 h. LCMS showed the starting material was consumed completely and one main peak with desired mass was detected. The reaction mixture was concentrated under reduced pressure to afford 4-([1,4′-bipiperidin]-4-ylethynyl)-2-(2,6-dioxopiperidin-3-yl)isoindoline-1,3-dione (80 mg, crude, HCl salt) as a light yellow solid, which was used directly for next step. MS(M+H)⁺=449.3

### Step 3. Synthesis of 4-((1′-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)-[1,4′-bipiperidin]-4-yl)ethynyl)-2-(2,6-dioxopiperidin-3-yl)isoindoline-1,3-dione (Compound 181)

To a solution of 4-([1,4′-bipiperidin]-4-ylethynyl)-2-(2,6-dioxopiperidin-3-yl)isoindoline-1,3-dione (80 mg, 164.96 umol, HCl salt) and 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (73.81 mg, 164.96 umol) in DMF (3 mL) were added HATU (94.08 mg, 247.44 umol) and DIEA (63.96 mg, 494.87 umol, 86.20 uL). The mixture was stirred at 25° C. for 5 h. LCMS showed 4-([1,4′-bipiperidin]-4-ylethynyl)-2-(2,6-dioxopiperidin-3-yl)isoindoline-1,3-dione was consumed completely and one main peak with desired mass was detected. The reaction mixture was diluted with water (50 mL) and extracted with EtOAc (20 mL×3). The combined organic layers were washed with brine (20 mL), dried over anhydrous sodium sulfate, filtered and concentrated under reduced pressure. The residue was purified by prep-HPLC (column: Phenomenex luna C18 150×25 mm×10 um; mobile phase: [water(FA)-ACN]; B %: 16%-46%, 10 min) followed by prep-HPLC (column: Waters Xbridge 150×25 mm×5 um; mobile phase: [water(NH₄HCO₃)-ACN]; B %: 48%-78%) to afford 4-((1′-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)-[1,4′-bipiperidin]-4-yl)ethynyl)-2-(2,6-dioxopiperidin-3-yl)isoindoline-1,3-dione (20.8 mg, 23.46 μmol, 14.22% yield, 99% purity) as a white solid. MS(M+H)⁺=878.1.

¹H NMR (400 MHz, DMSO-d) δ=11.13 (s, 1H), 8.24 (s, 1H), 8.14 (d, J=8.1 Hz, 1H), 8.00 (s, 1H), 7.90-7.78 (m, 3H), 7.05 (d, J=1.6 Hz, 1H), 6.97 (dd, J=1.6, 8.2 Hz, 1H), 5.15 (dd, J=5.4, 12.8 Hz, 1H), 4.76-4.65 (m, 1H), 4.02 (t, J=14.1 Hz, 2H), 3.89 (s, 3H), 3.33 (s, 3H), 2.94-2.79 (m, 4H), 2.77-2.71 (m, 1H), 2.64-2.59 (m, 1H), 2.57-2.52 (m, 5H), 2.43-2.32 (m, 2H), 2.11-2.02 (m, 1H), 1.92-1.85 (m, 4H), 1.83-1.73 (m, 2H), 1.71-1.63 (m, 4H), 1.60-1.51 (m, 4H), 1.48-1.36 (m, 2H).

### Example 182. Synthesis of 5-((1′-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoyl)-[1,4′-piperidin]-4-yl)ethynyl)-2-(2,6-dioxopiperidin-3-yl)isoindoline-1,3-dione (Compound 182)

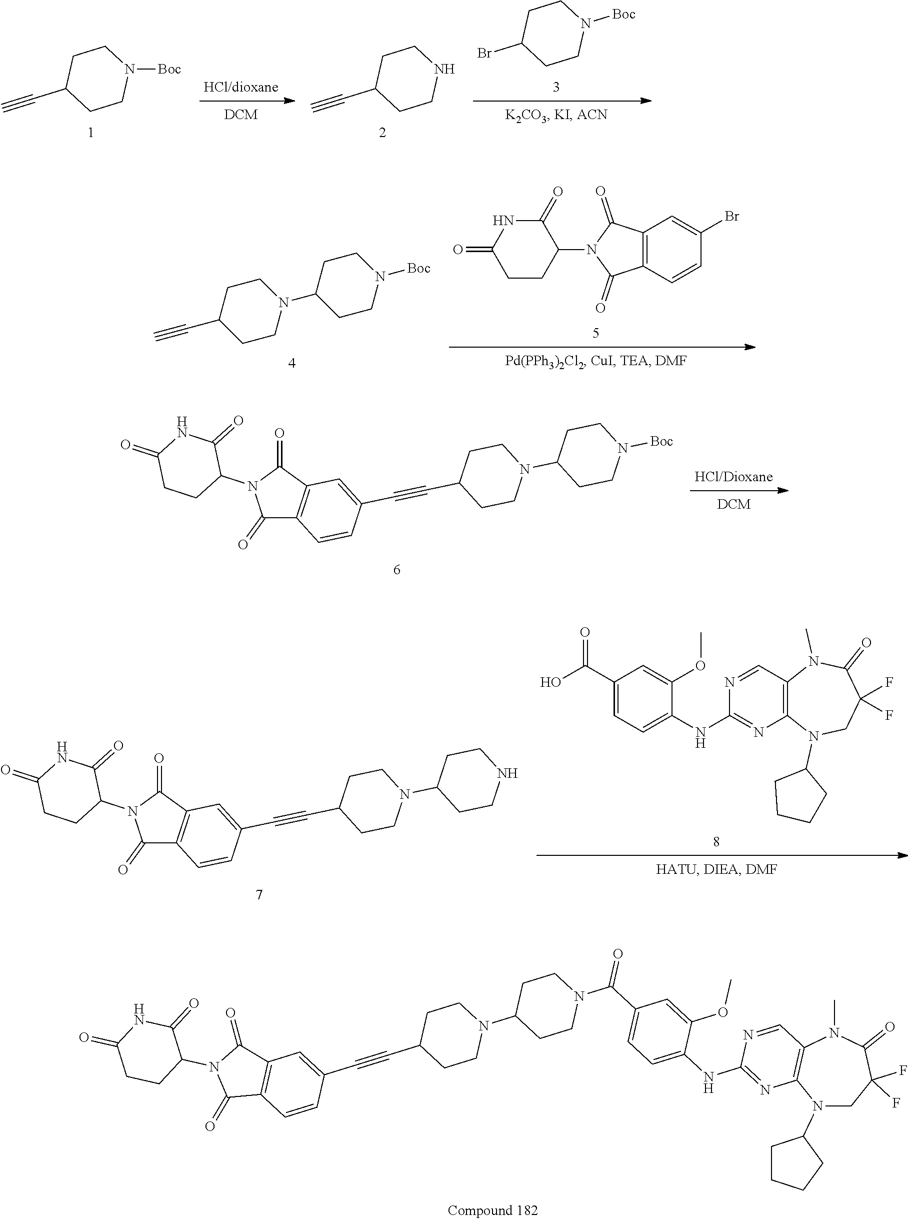

The compound 182 was synthesized by the method described in the scheme similar to the method described in Example 181.

MS(M+H)⁺=878.6, ¹H NMR (400 MHz, DMSO-d₆) δ=11.14 (s, 1H), 8.23 (s, 1H), 8.13 (d, J=8.2 Hz, 1H), 8.00 (s, 1H), 7.93-7.81 (m, 3H), 7.04 (d, J=1.3 Hz, 1H), 6.96 (dd, J=1.3, 8.3 Hz, 1H), 5.16 (dd, J=5.4, 12.8 Hz, 1H), 4.77-4.66 (m, 1H), 4.05-3.97 (m, 2H), 3.88 (s, 3H), 3.32 (s, 3H), 2.93-2.84 (m, 2H), 2.83-2.75 (m, 2H), 2.73-2.66 (m, 1H), 2.64-2.62 (m, 1H), 2.61-2.53 (m, 5H), 2.41-2.31 (m, 2H), 2.11-2.02 (m, 1H), 1.95-1.85 (m, 4H), 1.82-1.72 (m, 2H), 1.70-1.62 (m, 4H), 1.61-1.48 (m, 4H), 1.47-1.35 (m, 2H).

### Example 183. Synthesis of 4-((3-(4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoyl)piperazin-1-yl)propyl)amino)-2-(2,6-dioxopiperidin-3-yl)isoindoline-1,3-dione (Compound 183)

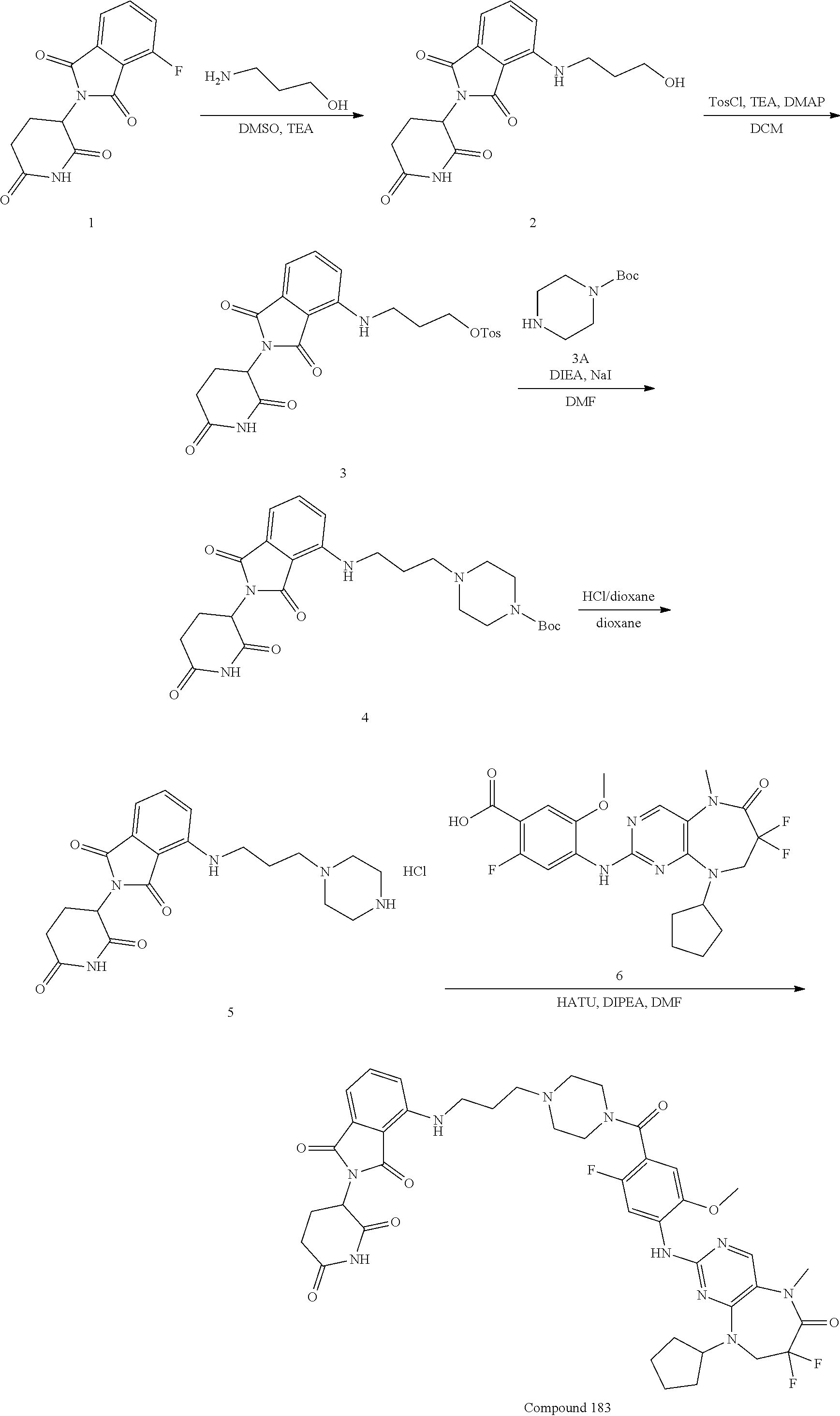

### Step 1. Synthesis of 2-(2,6-dioxopiperidin-3-yl)-4-((3-hydroxypropyl)amino)isoindoline-1,3-dione (2)

To a mixture of 2-(2,6-dioxopiperidin-3-yl)-4-fluoroisoindoline-1,3-dione (500 mg, 1.81 mmol) in DMSO (10 mL) were added 3-aminopropan-1-ol (203.94 mg, 2.72 mmol, 209.38 μL) and TEA (549.50 mg, 5.43 mmol, 755.85 μL) at 25° C. The mixture was stirred at 90° C. for 2 h. TLC (petroleum ether:EtOAc=1:2) indicated one new spot formed. The reaction mixture was diluted with water (50 mL) and extracted with EtOAc (50 mL×3). The combined organic layers were washed with saturated brine (50 mL), dried over anhydrous sodium sulfate, filtered and concentrated under reduced pressure to afford 2-(2,6-dioxopiperidin-3-yl)-4-((3-hydroxypropyl)amino)isoindoline-1,3-dione (0.2 g, 301.82 μmol, 16.67% yield, 50% purity) as yellow oil. MS(M+H)⁺=332.1.

### Step 2. Synthesis of 3-((2-(2,6-dioxopiperidin-3-yl)-1,3-dioxoisoindolin-4-yl)amino)propyl 4-methylbenzenesulfonate (3)

To a mixture of 2-(2,6-dioxopiperidin-3-yl)-4-((3-hydroxypropyl)amino)isoindoline-1,3-dione (200 mg, 301.82 μmol, 50% purity) in DCM (10 mL) were added TosCl (115.08 mg, 603.64 μmol), TEA (91.62 mg, 905.46 μmol, 126.03 μL) and DMAP (7.37 mg, 60.36 μmol) at 25° C. The mixture was stirred at 25° C. for 12 h. TLC (petroleum ether:EtOAc=1:1) indicated one new spot formed. The reaction mixture (combined with another batch) was concentrated under reduced pressure to remove DCM. The residue was diluted with water (50 mL) and extracted with EtOAc (50 mL×3). The combined organic layers were washed with brine (50 mL), dried over anhydrous sodium sulfate, filtered and concentrated under reduced pressure to give a residue. The residue was purified by flash silica gel chromatography (ISCO®; 10 g Sepa Flash® Silica Flash Column, Eluent of 0˜80% petroleum ether:EtOAc gradient @50 mL/min) to afford 3-((2-(2,6-dioxopiperidin-3-yl)-1,3-dioxoisoindolin-4-yl)amino)propyl 4-methylbenzenesulfonate (160 mg, 322.96 μmol, 71.34% yield, 98% purity) as yellow oil. MS(M+H)⁺=486.0

### Step 3. Synthesis of tert-butyl 4-(3-((2-(2,6-dioxopiperidin-3-yl)-1,3-dioxoisoindolin-4-yl)amino)propyl)piperazine-1-carboxylate (4)

To a solution of 3-((2-(2,6-dioxopiperidin-3-yl)-1,3-dioxoisoindolin-4-yl)amino)propyl 4-methylbenzenesulfonate (160 mg, 329.55 μmol) in DMF (5 mL) were added DIEA (127.78 mg, 988.65 μmol, 172.21 μL), tert-butyl piperazine-1-carboxylate (73.65 mg, 395.46 μmol) and NaI (9.88 mg, 65.91 μmol) at 25° C. The mixture was stirred at 70° C. for 12 h. LCMS showed ˜97% of desired mass was detected. The reaction mixture was diluted with water (50 mL) and extracted with EtOAc (50 mL×4). The combined organic layers were washed with saturated brine (100 mL), dried over anhydrous sodium sulfate, filtered and concentrated under reduced pressure to afford tert-butyl 4-[3-[[2-(2,6-dioxo-3-piperidyl)-1,3-dioxo-isoindolin-4-yl]amino]propyl]piperazine-1-carboxylate (160 mg, 294.66 μmol, 89.41% yield, 92% purity) as yellow oil. MS(M+H)⁺=500.3

### Step 4. Synthesis of 2-(2,6-dioxopiperidin-3-yl)-4-((3-(piperazin-1-yl)propyl)amino)isoindoline-1,3-dione (5)

To a solution of tert-butyl 4-(3-((2-(2,6-dioxopiperidin-3-yl)-1,3-dioxoisoindolin-4-yl)amino)propyl)piperazine-1-carboxylate (160 mg, 320.28 μmol) in dioxane (1 mL) was added HCl/dioxane (4 M, 1 mL) at 25° C. The mixture was stirred at 25° C. for 1 h. TLC (EtOAc:methanol=10:1) indicated one new spot formed. The mixture was concentrated under reduced pressure to give 2-(2,6-dioxopiperidin-3-yl)-4-((3-(piperazin-1-yl)propyl)amino)isoindoline-1,3-dione (130 mg, 167.01 μmol, 52.14% yield, 56% purity, HCl) as a yellow solid. MS(M+H)⁺=400.1

### Step 5. Synthesis of 4-((3-(4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoyl)piperazin-1-yl)propyl)amino)-2-(2,6-dioxopiperidin-3-yl)isoindoline-1,3-dione (Compound 183)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (48.53 mg, 104.28 μmol) in DMF (2 mL) were added HATU (79.30 mg, 208.55 μmol) and DIPEA (67.39 mg, 521.38 umol, 90.82 μL) at 25° C. 2-(2,6-dioxopiperidin-3-yl)-4-((3-(piperazin-1-yl)propyl)amino)isoindoline-1,3-dione (50 mg, 114.70 μmol, HCl) was added to the mixture after 0.5 h. The mixture was stirred at 25° C. for 14 h. LCMS showed ˜50% of desired mass was detected. The reaction mixture was diluted with water (50 mL) and extracted with EtOAc (50 mL×4). The combined organic layers were washed with saturated brine (50 mL), dried over anhydrous sodium sulfate, filtered and concentrated under reduced pressure to give a residue. The residue was purified by prep-HPLC (column: YMC Triart 30×150 mm×7 μm; mobile phase: [water(HCl)-ACN]; B %: 26%-46%, 7 min) and prep-HPLC (column: Waters Xbridge 150×25 mm×5 μm; mobile phase: [water(NH₄HCO₃)-ACN]; B %: 48%-78%, 10 min) followed by lyophilization to afford 4-((3-(4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoyl)piperazin-1-yl)propyl)amino)-2-(2,6-dioxopiperidin-3-yl)isoindoline-1,3-dione (18.6 mg, 21.77 μmol, 20.87% yield, 99.1% purity) as a yellow solid. MS(M+H)⁺=847.3

¹H NMR (400 MHz, DMSO-d₆) δ=11.10 (br. s, 1H), 8.27 (s, 1H), 8.11 (d, J=12 Hz, 1H), 8.04 (s, 1H), 7.58 (t, J=8.0 Hz, 1H), 7.12 (d, J=8.8 Hz, 1H), 7.02-6.97 (m, 2H), 6.82 (t, J=5.6 Hz, 1H), 5.04 (dd, J=5.2, 12.8 Hz, 1H), 4.79-4.75 (m, 1H), 4.06 (t, J=14 Hz, 2H), 3.88 (s, 3H), 3.70-3.65 (m, 2H), 3.43-3.40 (m, 2H), 3.32-3.31 (m, 5H), 2.92-2.88 (m, 1H), 2.59-2.55 (m, 1H), 2.42-2.33 (m, 6H), 1.76-1.23 (m, 12H).

### Example 184. Synthesis of 4-((4-(4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoyl)piperazin-1-yl)butyl)amino)-2-(2,6-dioxopiperidin-3-yl)isoindoline-1,3-dione (Compound 184)

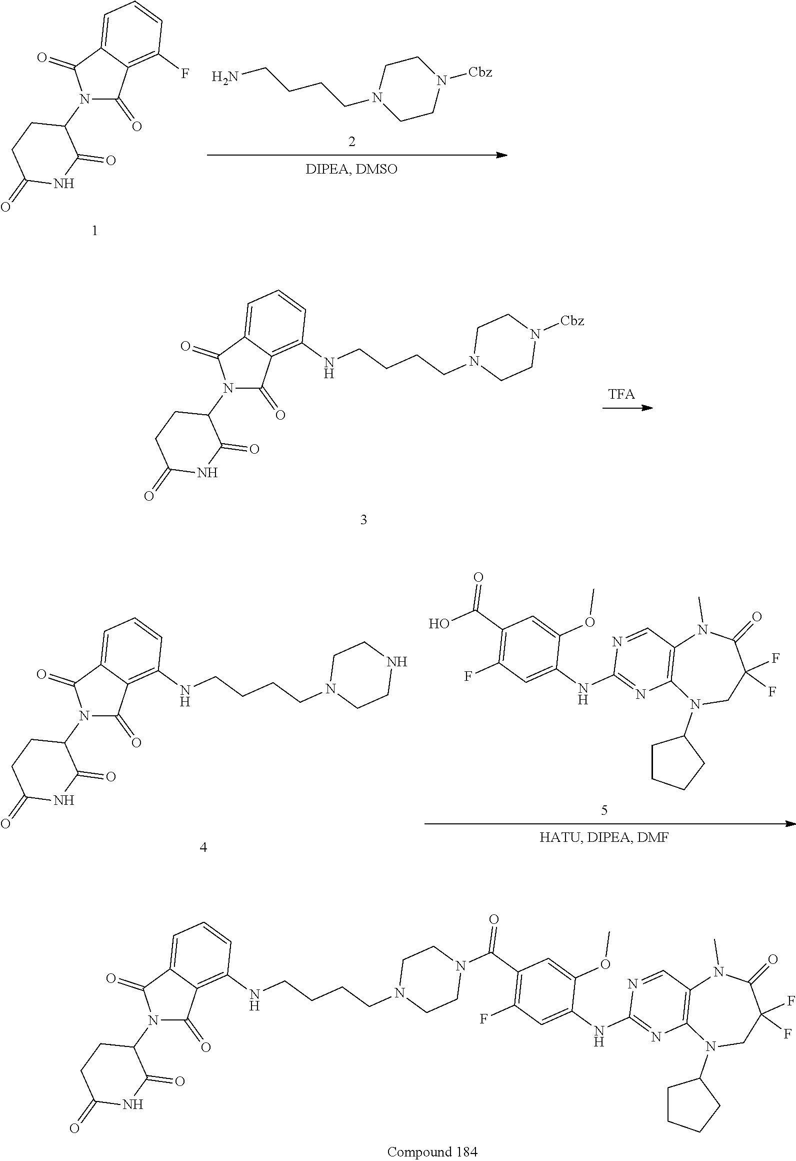 [SMILES: O=C1CCC(N2C(=O)c3cccc(F)c3C2=O)C(=O)N1 ; NCCCCN1CCN(C(=O)OCc2ccccc2)CC1 ; O=C1CCC(N2C(=O)c3cccc(NCCCCN4CCN(C(=O)OCc5ccccc5)CC4)c3C2=O)C(=O)N1 ; O=C1CCC(N2C(=O)c3cccc(NCCCCN4CCNCC4)c3C2=O)C(=O)N1 ; COc1cc(C(=O)O)c(F)cc1Nc1ncc2c(n1)N(C1CCCC1)CC(F)(F)C(=O)N2C ; COc1cc(C(=O)N2CCN(CCCCNc3cccc4c3C(=O)N(C3CCC(=O)NC3=O)C4=O)CC2)c(F)cc1Nc1ncc2c(n1)N(C1CCCC1)CC(F)(F)C(=O)N2C]

The compound 184 was synthesized by the method described in the scheme similar to the methods described in Examples 1 and 183.

MS(M+H)⁺=861.1, ¹H NMR (400 MHz, DMSO-d₆) δ=11.07 (s, 1H), 8.28 (s, 1H), 8.18 (d, J=12.1 Hz, 1H), 8.04 (s, 1H), 7.63-7.54 (m, 1H), 7.12 (d, J=8.6 Hz, 1H), 7.01 (dd, J=6.6, 16.9 Hz, 2H), 6.57 (t, J=5.8 Hz, 1H), 5.05 (dd, J=5.4, 12.9 Hz, 1H), 4.85-4.72 (m, 1H), 4.07 (t, J=13.9 Hz, 2H), 3.89 (s, 3H), 3.68-3.56 (m, 2H), 3.32-3.25 (m, 7H), 2.95-2.81 (m, 1H), 2.63-2.53 (m, 2H), 2.45-2.30 (m, 6H), 2.07-1.88 (m, 3H), 1.74-1.48 (m, 10H).

### Example 185. Synthesis of 4-((5-(4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoyl)piperazin-1-yl)pentyl)amino)-2-(2,6-dioxopiperidin-3-yl)isoindoline-1,3-dione (Compound 185)

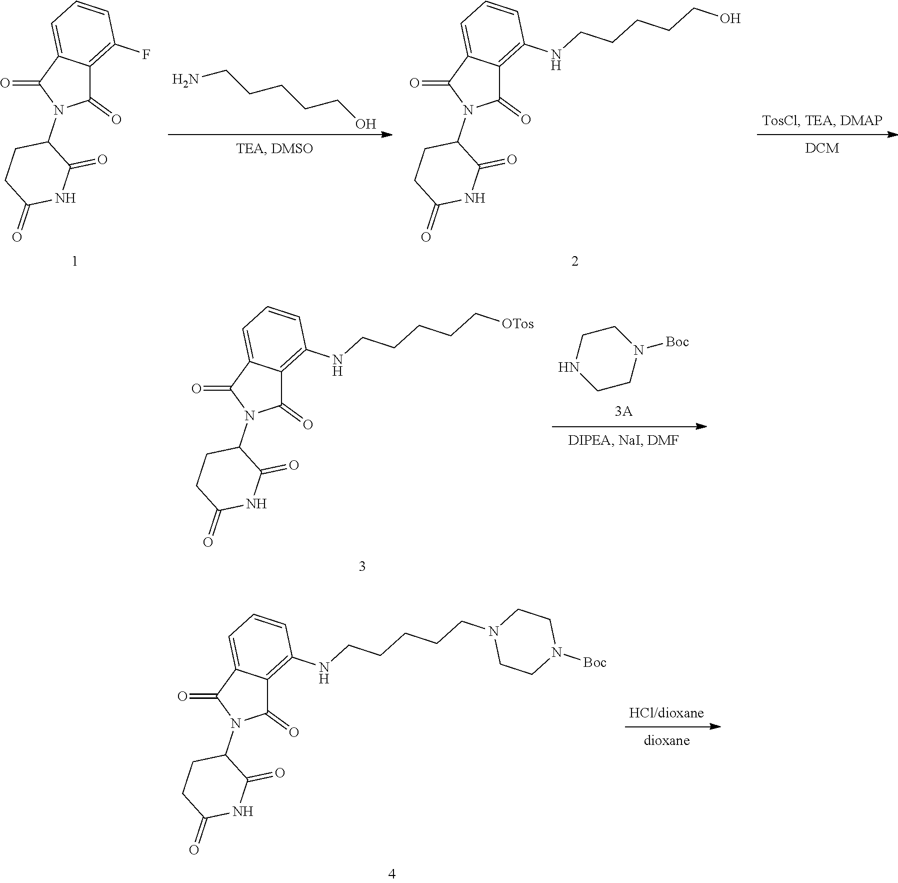

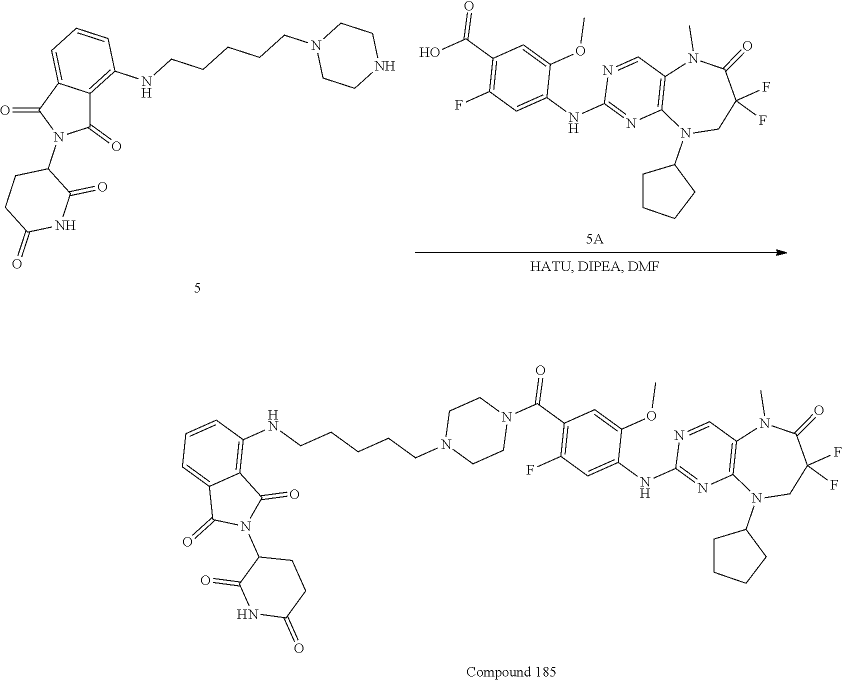 [SMILES: O=C1CCC(N2C(=O)c3cccc(NCCCCCN4CCNCC4)c3C2=O)C(=O)N1 ; COc1cc(C(=O)O)c(F)cc1Nc1ncc2c(n1)N(C1CCCC1)CC(F)(F)C(=O)N2C ; COc1cc(C(=O)N2CCN(CCCCCNc3cccc4c3C(=O)N(C3CCC(=O)NC3=O)C4=O)CC2)c(F)cc1Nc1ncc2c(n1)N(C1CCCC1)CC(F)(F)C(=O)N2C]

The compound 185 was synthesized by the method described in the scheme similar to the method described in Example 183.

MS(M+H)⁺=875.1, ¹H NMR (400 MHz, DMSO-d₆) δ=11.09 (br s, 1H), 8.29 (d, J=1.6 Hz, 1H), 8.18 (br d, J=11.6 Hz, 1H), 8.04 (br s, 1H), 7.63-7.54 (m, 1H), 7.10 (br d, J=7.6 Hz, 1H), 7.00 (br dd, J=5.4, 17.1 Hz, 2H), 6.54 (br s, 1H), 5.05 (br d, J=8.5 Hz, 1H), 4.85-4.73 (m, 1H), 4.12-4.01 (m, 2H), 3.89 (br d, J=1.0 Hz, 3H), 3.52-3.69 (m, 2H), 3.21-3.32 (m, 7H), 2.95-2.83 (m, 1H), 2.59-2.68 (m, 1H), 2.44-2.27 (m, 6H), 2.08-1.88 (m, 3H), 1.77-1.30 (m, 13H).

### Example 186. Synthesis of 5-((3-(4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoyl)piperazin-1-yl)propyl)amino)-2-(2,6-dioxopiperidin-3-yl)isoindoline-1,3-dione (Compound 186)

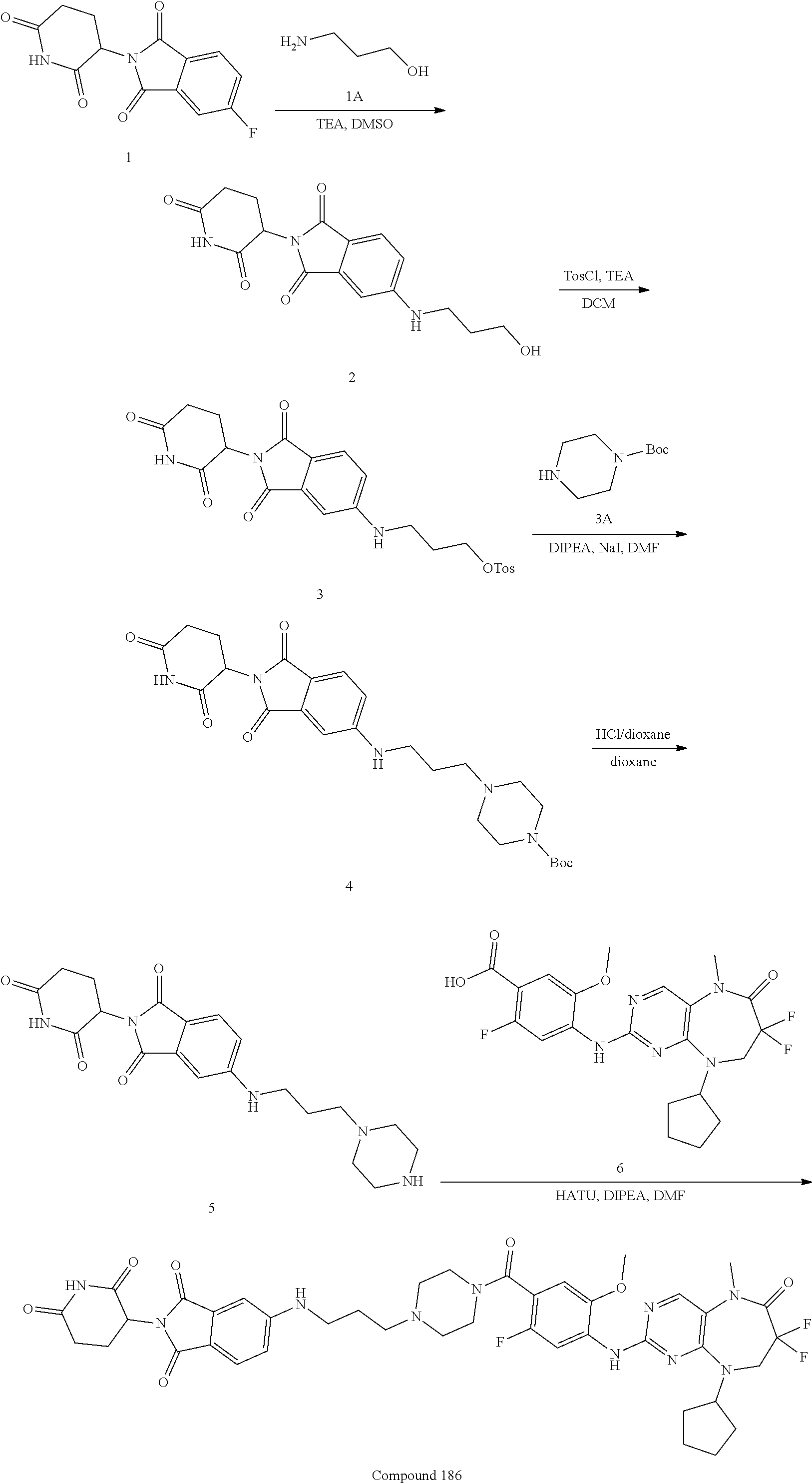

The compound 186 was synthesized by the method described in the scheme similar to the method described in Example 183.

MS(M+H)⁺=847.1, ¹H NMR (400 MHz, DMSO-d₆) δ=11.05 (s, 1H), 8.28 (s, 1H), 8.17 (d, J=12.0 Hz, 1H), 8.03 (s, 1H), 7.56 (d, J=8.4 Hz, 1H), 7.14 (t, J=5.3 Hz, 1H), 7.01-6.95 (m, 2H), 6.85 (dd, J=1.9, 8.4 Hz, 1H), 5.02 (dd, J=5.3, 12.9 Hz, 1H), 4.82-4.72 (m, 1H), 4.06 (t, J=13.9 Hz, 2H), 3.88 (s, 3H), 3.70-3.62 (m, 2H), 3.32-3.29 (m, 5H), 3.26-3.17 (m, 2H), 2.93-2.81 (m, 1H), 2.61-2.54 (m, 1H), 2.47-2.38 (m, 4H), 2.38-2.31 (m, 2H), 2.03-1.89 (m, 3H), 1.78-1.51 (m, 9H).

### Example 187. Synthesis of 5-((4-(4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoyl)piperazin-1-yl)butyl)amino)-2-(2,6-dioxopiperidin-3-yl)isoindoline-1,3-dione (Compound 187)

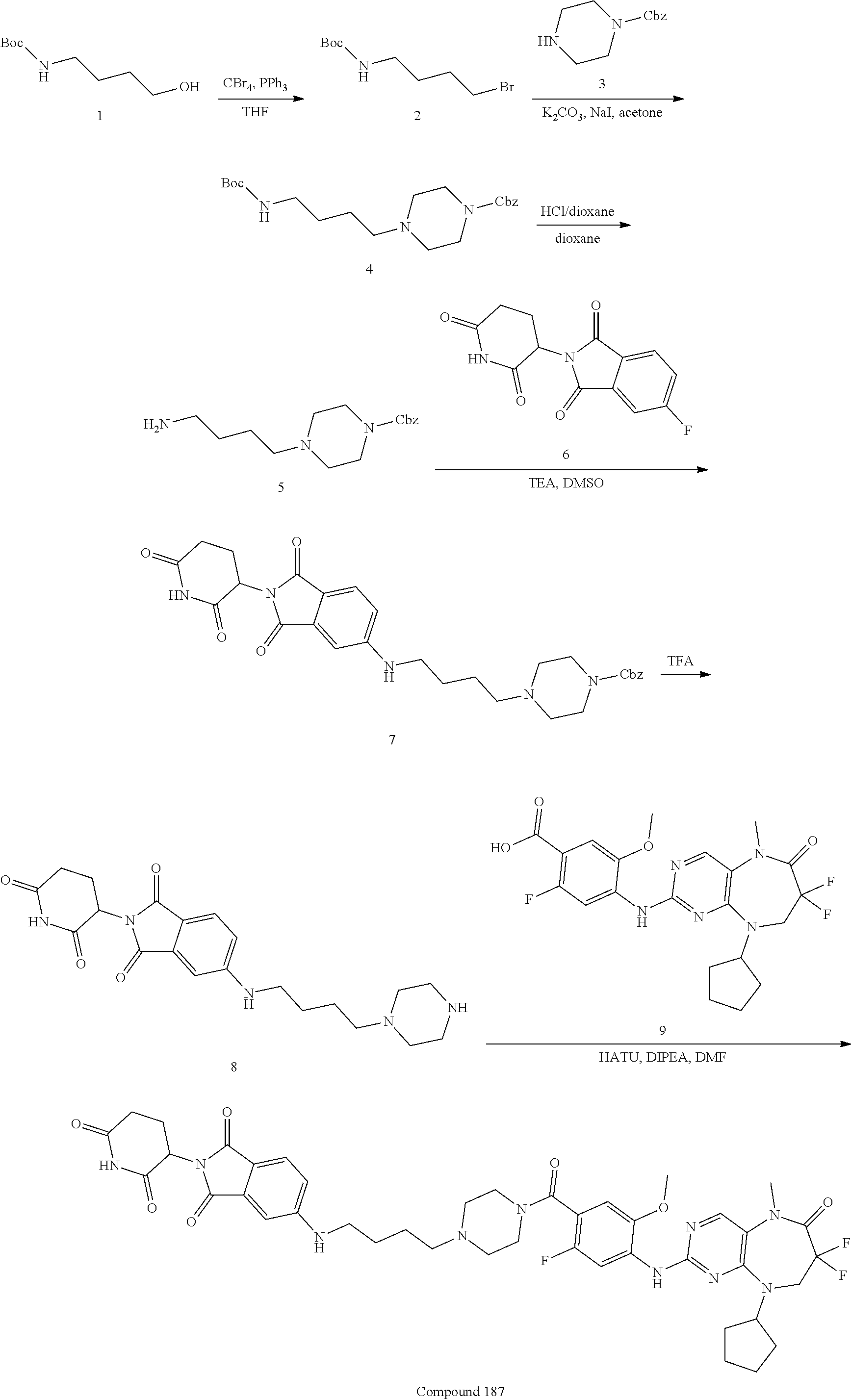

The compound 187 was synthesized by the method described in the scheme similar to the method described in Example 183.

MS(M+H)⁺=861.1, ¹H NMR (400 MHz, DMSO-d₆) δ=11.07 (s, 1H), 8.28 (s, 1H), 8.17 (d, J=12.1 Hz, 1H), 8.05 (s, 1H), 7.56 (d, J=8.4 Hz, 1H), 7.13 (t, J=5.2 Hz, 1H), 7.01-6.94 (m, 2H), 6.85 (dd, J=2.0, 8.4 Hz, 1H), 5.03 (dd, J=5.3, 12.9 Hz, 1H), 4.84-4.73 (m, 1H), 4.06 (t, J=13.9 Hz, 2H), 3.88 (s, 3H), 3.72-3.58 (m, 2H), 3.35-3.27 (m, 5H), 3.23-3.14 (m, 2H), 2.94-2.81 (m, 1H), 2.63-2.53 (m, 2H), 2.50-2.25 (m, 6H), 2.04-1.87 (m, 3H), 1.74-1.51 (m, 10H).

### Example 188. Synthesis of 5-((5-(4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoyl)piperazin-1-yl)pentyl)amino)-2-(2,6-dioxopiperidin-3-yl)isoindoline-1,3-dione (Compound 188)

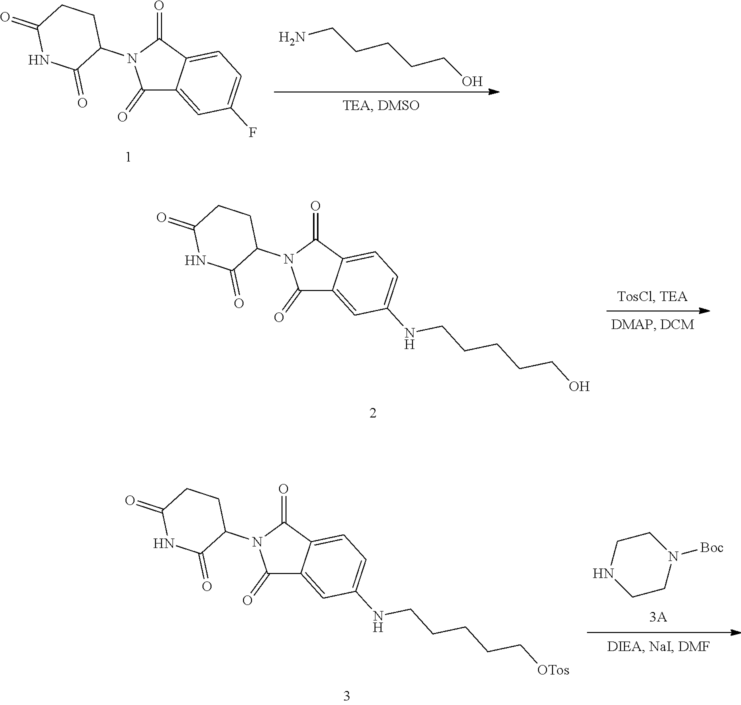

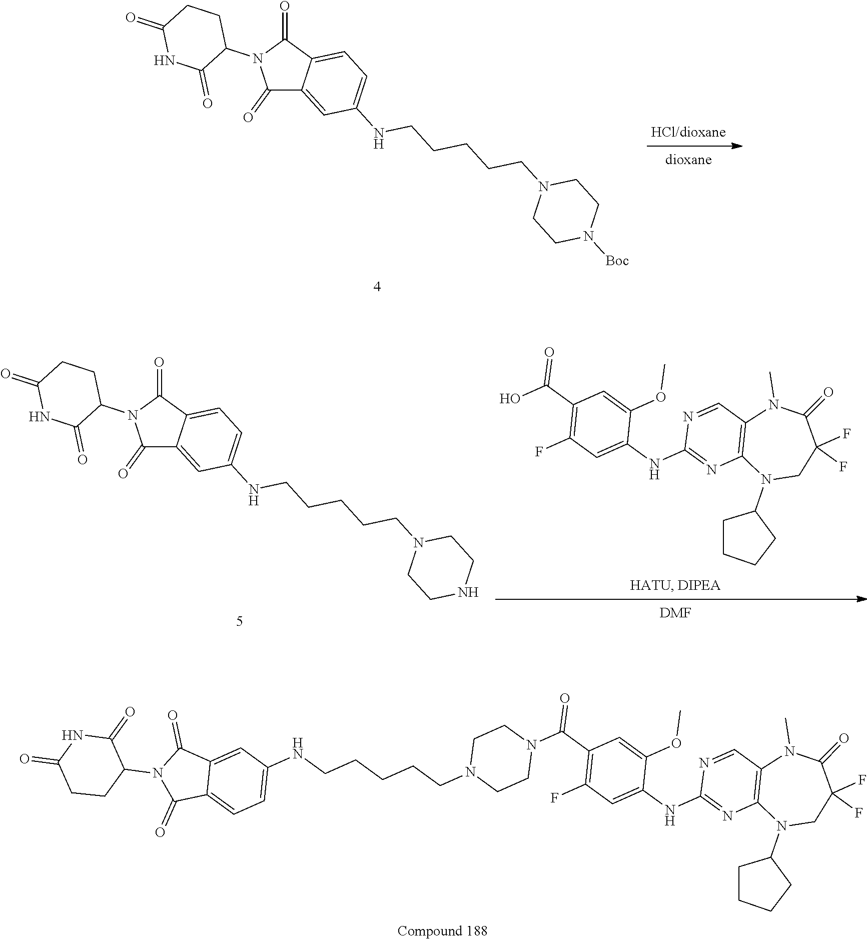

The compound 188 was synthesized by the method described in the scheme similar to the method described in Example 183.

MS(M+H)⁺=875.1, ¹H NMR (400 MHz, DMSO-d₆) δ=11.05 (s, 1H), 8.28 (s, 1H), 8.17 (d, J=12.1 Hz, 1H), 8.04 (s, 1H), 7.56 (d, J=8.3 Hz, 1H), 7.11 (t, J=5.0 Hz, 1H), 7.00-6.92 (m, 2H), 6.85 (dd, J=1.9, 8.4 Hz, 1H), 5.03 (dd, J=5.3, 12.9 Hz, 1H), 4.79 (q, J=8.0 Hz, 1H), 4.06 (t, J=13.9 Hz, 2H), 3.89 (s, 3H), 3.52-3.71 (m, 2H), 3.29-3.38 (m, 5H), 3.21-3.10 (m, 2H), 2.95-2.79 (m, 1H), 2.59-2.63 (m, 1H), 2.45-2.38 (m, 2H), 2.38-2.25 (m, 4H), 2.05-1.87 (m, 3H), 1.81-1.28 (m, 13H).

### Example 189. Synthesis of 4-((2-(2-(4-(1-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoyl)piperidin-4-yl)piperazin-1-yl)ethoxy)ethyl)amino)-2-(2,6-dioxopiperidin-3-yl)isoindoline-1,3-dione (Compound 189)

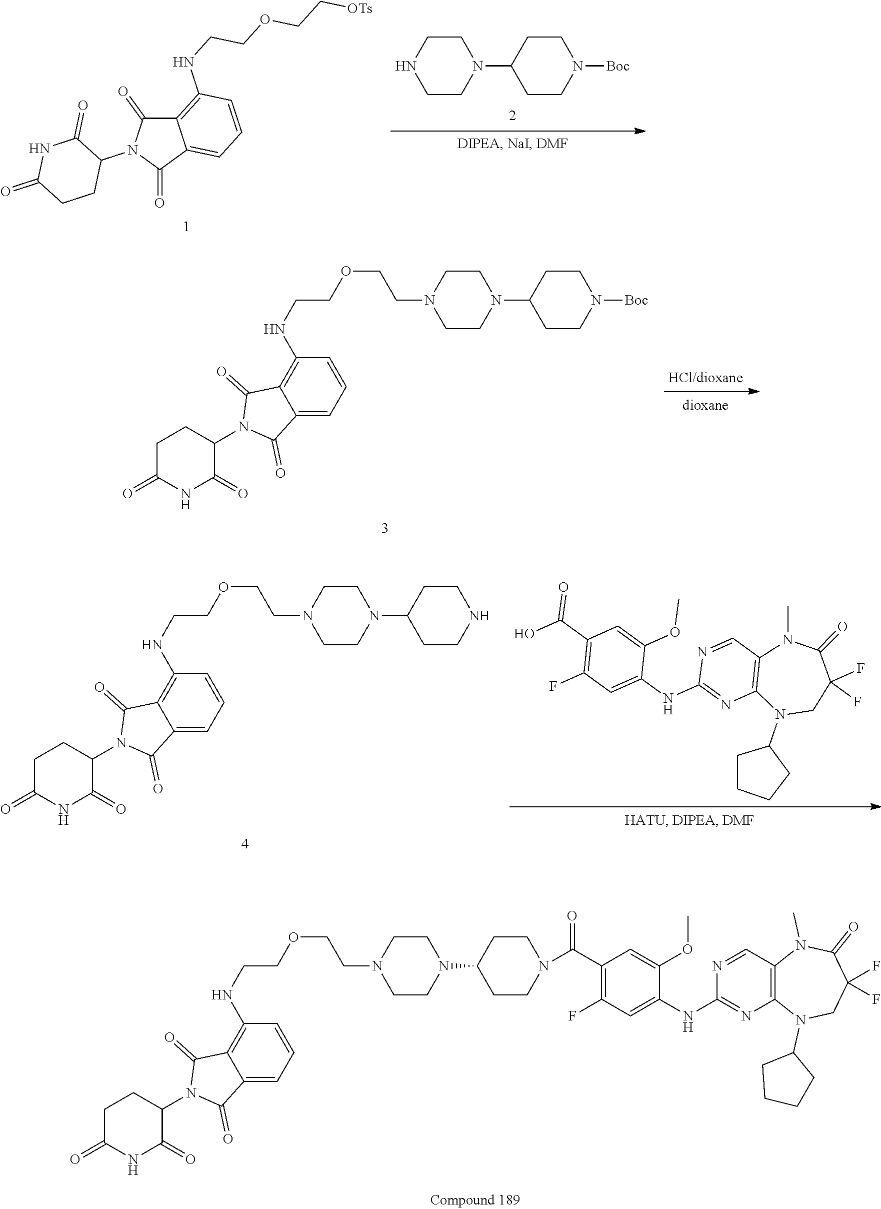

### Step 1. Synthesis of tert-butyl 4-(4-(2-(2-((2-(2,6-dioxopiperidin-3-yl)-1,3-dioxoisoindolin-4-yl)amino) ethoxy)ethyl) piperazin-1-yl) piperidine-1-carboxylate (3)

To a solution of 2-(2-((2-(2,6-dioxopiperidin-3-yl)-1,3-dioxoisoindolin-4-yl)amino)ethoxy)ethyl 4-methylbenzenesulfonate (150 mg, 290.96 μmol) and tert-butyl 4-piperazin-1-ylpiperidine-1-carboxylate (86.22 mg, 320.06 μmol) in DMF (6 mL) were added NaI (8.72 mg, 58.19 μmol) and DIPEA (112.81 mg, 872.88 μmol, 152.04 L) at 20° C. and the resulting mixture was stirred at 80° C. for 16 h. LCMS showed 2-(2-((2-(2,6-dioxopiperidin-3-yl)-1,3-dioxoisoindolin-4-yl)amino)ethoxy)ethyl 4-methylbenzenesulfonate was consumed completely and 73% peak with desired mass was detected. The reaction mixture was diluted with H₂O (15 mL) and extracted with EtOAc (15 mL×3). The organic layer was washed with brine (15 mL×3), dried over Na₂SO₄, filtered and concentrated. The residue was purified by flash silica gel chromatography (10 g SepaFlash® Silica Flash Column, Eluent of 0-100% EtOAc/Petroleum ether to 0˜10% Dichloromethane/Methanol gradient @100 mL/min) to afford tert-butyl 4-(4-(2-(2-((2-(2,6-dioxopiperidin-3-yl)-1,3-dioxoisoindolin-4-yl)amino)ethoxy)ethyl) piperazin-1-yl)piperidine-1-carboxylate (129 mg, 202.12 μmol, 69.47% yield, 96% purity) as a yellow oil. MS(M+H)⁺=613.2

### Step 2. Synthesis of 2-(2,6-dioxopiperidin-3-yl)-4-((2-(2-(4-(piperidin-4-yl)piperazin-1-yl)ethoxy)ethyl)amino)isoindoline-1,3-dione (4)

To a solution of tert-butyl 4-(4-(2-(2-((2-(2,6-dioxopiperidin-3-yl)-1,3-dioxoisoindolin-4-yl)amino)ethoxy)ethyl) piperazin-1-yl)piperidine-1-carboxylate (129 mg, 210.54 μmol) in dioxane (1 mL) was added HCl/dioxane (4 M, 9.44 mL) at 20° C. and the resulting mixture was stirred at 20° C. for 0.5 h. LCMS showed starting material was consumed completely and 92% peak with desired mass was detected. The reaction mixture was concentrated in vacuum to afford 2-(2,6-dioxopiperidin-3-yl)-4-((2-(2-(4-(piperidin-4-yl)piperazin-1-yl)ethoxy)ethyl)amino)isoindoline-1,3-dione (120 mg, crude, HCl) as a yellow solid. MS(M+H)⁺=513.3

### Step 3. Synthesis of 4-((2-(2-(4-(1-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoyl)piperidin-4-yl)piperazin-1-yl)ethoxy)ethyl)amino)-2-(2,6-dioxopiperidin-3-yl)isoindoline-1,3-dione (Compound 189)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (90 mg, 193.37 μmol) in DMF (4 mL) were added HATU (80.88 mg, 212.71 μmol) and DIPEA (49.98 mg, 386.74 μmol, 67.36 μL). The mixture was stirred at 20° C. for 10 min and a solution of 2-(2,6-dioxopiperidin-3-yl)-4-((2-(2-(4-(piperidin-4-yl)piperazin-1-yl)ethoxy)ethyl)amino)isoindoline-1,3-dione (116.79 mg, 212.71 μmol, HCl) in DMF (4 mL) and DIPEA (49.98 mg, 386.74 μmol, 67.36 μL) was added and the resulting mixture was stirred at 20° C. for 1 h. LCMS showed all starting material was consumed completely and 72% peak with desired mass was detected. The reaction mixture was diluted with H₂O (12 mL) and extracted with EtOAc (12 mL×3). The organic layer was washed with brine (12 mL×3), dried over Na₂SO₄, filtered and concentrated. The residue was purified by prep-HPLC (column: Phenomenex Synergi C18 150*25 mm*10 um; mobile phase: [water(FA)-ACN]; B %: 15%-45%, 10 min) and re-purified by prep-HPLC (column: Waters Xbridge 150*25 mm*5 um; mobile phase: [water(NH₄HCO₃)-ACN]; B %: 43%-73%, 8 min) and lyophilization to afford 4-((2-(2-(4-(1-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoyl)piperidin-4-yl)piperazin-1-yl)ethoxy)ethyl)amino)-2-(2,6-dioxopiperidin-3-yl)isoindoline-1,3-dione (34.1 mg, 35.17 μmol, 18.19% yield, 99% purity) as a yellow solid. MS(M+H)⁺=959.9

¹H NMR (400 MHz, DMSO-d₆) δ=11.09 (br s, 1H), 8.26 (s, 1H), 8.15 (d, J=12.0 Hz, 1H), 8.03 (s, 1H), 7.61-7.54 (m, 1H), 7.14 (d, J=8.6 Hz, 1H), 7.03 (d, J=7.1 Hz, 1H), 6.97 (d, J=6.1 Hz, 1H), 6.58 (br t, J=5.7 Hz, 1H), 5.04 (dd, J=5.4, 12.8 Hz, 1H), 4.77 (q, J=7.9 Hz, 1H), 4.49-4.40 (m, 1H), 4.05 (t, J=13.9 Hz, 2H), 3.87 (s, 3H), 3.60-3.56 (m, 2H), 3.52 (t, J=5.6 Hz, 3H), 3.47-3.44 (m, 2H), 3.32 (s, 3H), 3.10-2.98 (m, 1H), 2.92-2.76 (m, 2H), 2.60-2.53 (m, 2H), 2.47-2.31 (m, 11H), 2.06-1.97 (m, 1H), 1.97-1.89 (m, 2H), 1.82-1.79 (m, 1H), 1.75-1.69 (m, 3H), 1.65-1.52 (m, 4H), 1.36-1.22 (m, 2H).

### Example 190. Synthesis of 4-((3-(2-(4-(1-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoyl)piperidin-4-yl)piperazin-1-yl)ethoxy)propyl)amino)-2-(2,6-dioxopiperidin-3-yl)isoindoline-1,3-dione (Compound 190)

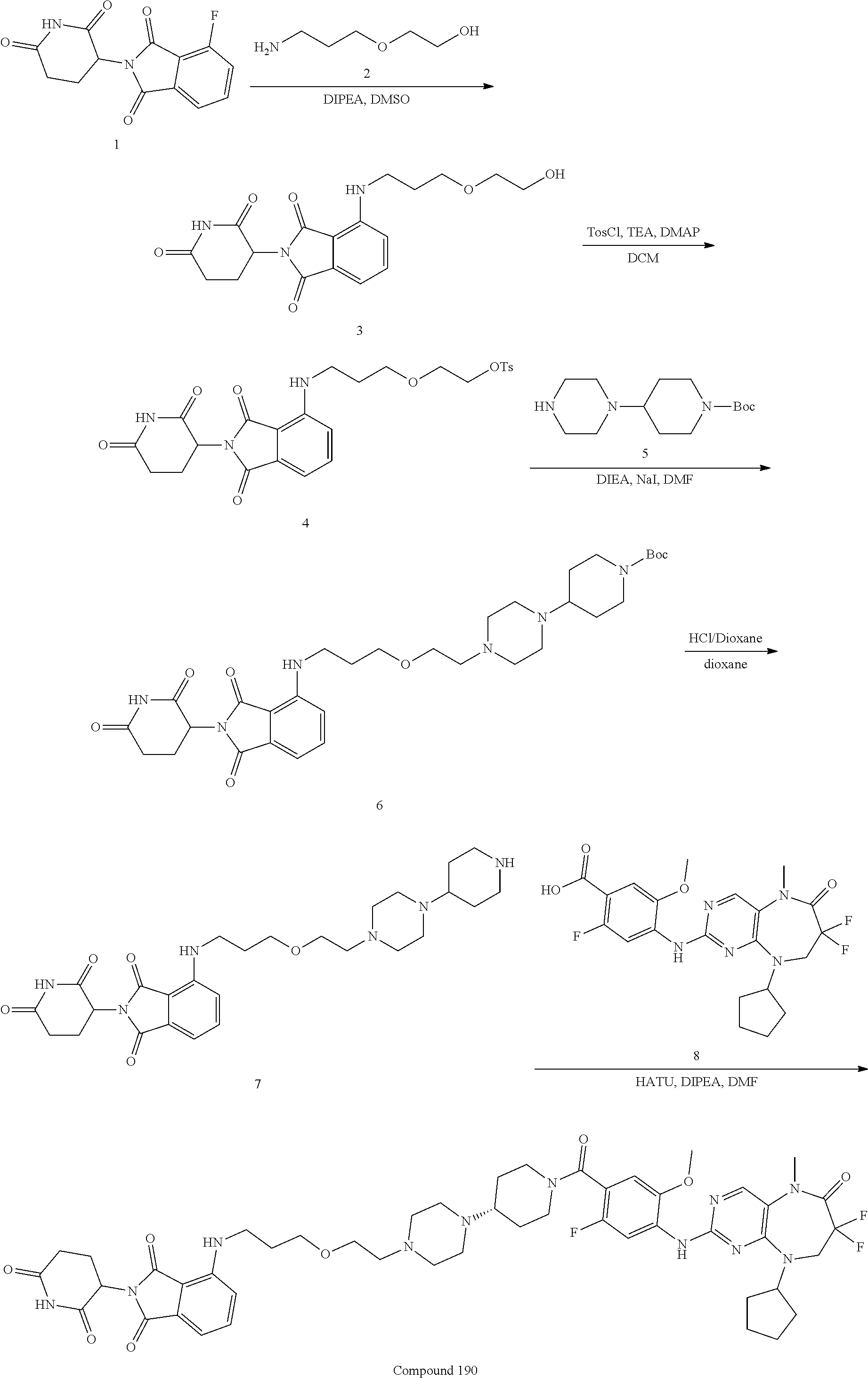

The compound 190 was synthesized by the method described in the scheme similar to the method described in Example 189.

MS (M+H)⁺=974.5, ¹H NMR (400 MHz, DMSO-d₆) δ=11.08 (s, 1H), 8.27 (s, 1H), 8.15 (d, J=12.0 Hz, 1H), 8.02 (s, 1H), 7.57 (t, J=8.0 Hz, 1H), 7.08 (d, J=8 Hz, 1H), 7.02 (d, J=8.0 Hz, 1H), 6.98 (d, J=8.0 Hz, 1H), 6.65 (t, J=4.0 Hz, 1H), 5.06-5.02 (m, 1H), 4.79-4.75 (m, 1H), 4.46-4.43 (m, 1H), 4.05 (t, J=12.0 Hz, 2H), 3.87 (s, 3H), 3.52-3.51 (m, 1H), 3.48-3.45 (m, 4H), 3.37-3.34 (m, 2H), 3.29 (s, 3H), 3.06-3.00 (m, 1H), 2.92-2.77 (m, 2H), 2.66-2.55 (m, 4H), 2.45-2.35 (m, 9H), 2.03-2.00 (m, 1H), 1.94-1.92 (m, 2H), 1.83-1.77 (m, 3H), 1.73-1.66 (m, 3H), 1.64-1.54 (m, 4H), 1.33-1.27 (m, 2H).

### Example 191. Synthesis of 5-((2-(2-(4-(1-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoyl)piperidin-4-yl)piperazin-1-yl)ethoxy)ethyl)amino)-2-(2,6-dioxopiperidin-3-yl)isoindoline-1,3-dione (Compound 191)

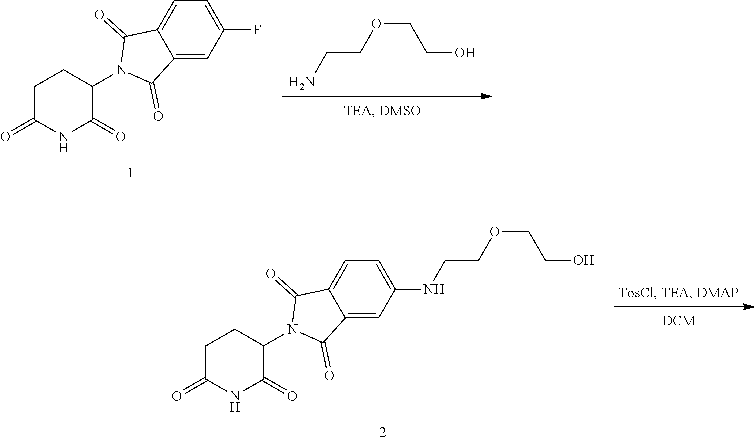 [SMILES: O=C1CCC(N2C(=O)c3ccc(F)cc3C2=O)C(=O)N1 ; NCCOCCO ; O=C1CCC(N2C(=O)c3ccc(NCCOCCO)cc3C2=O)C(=O)N1]

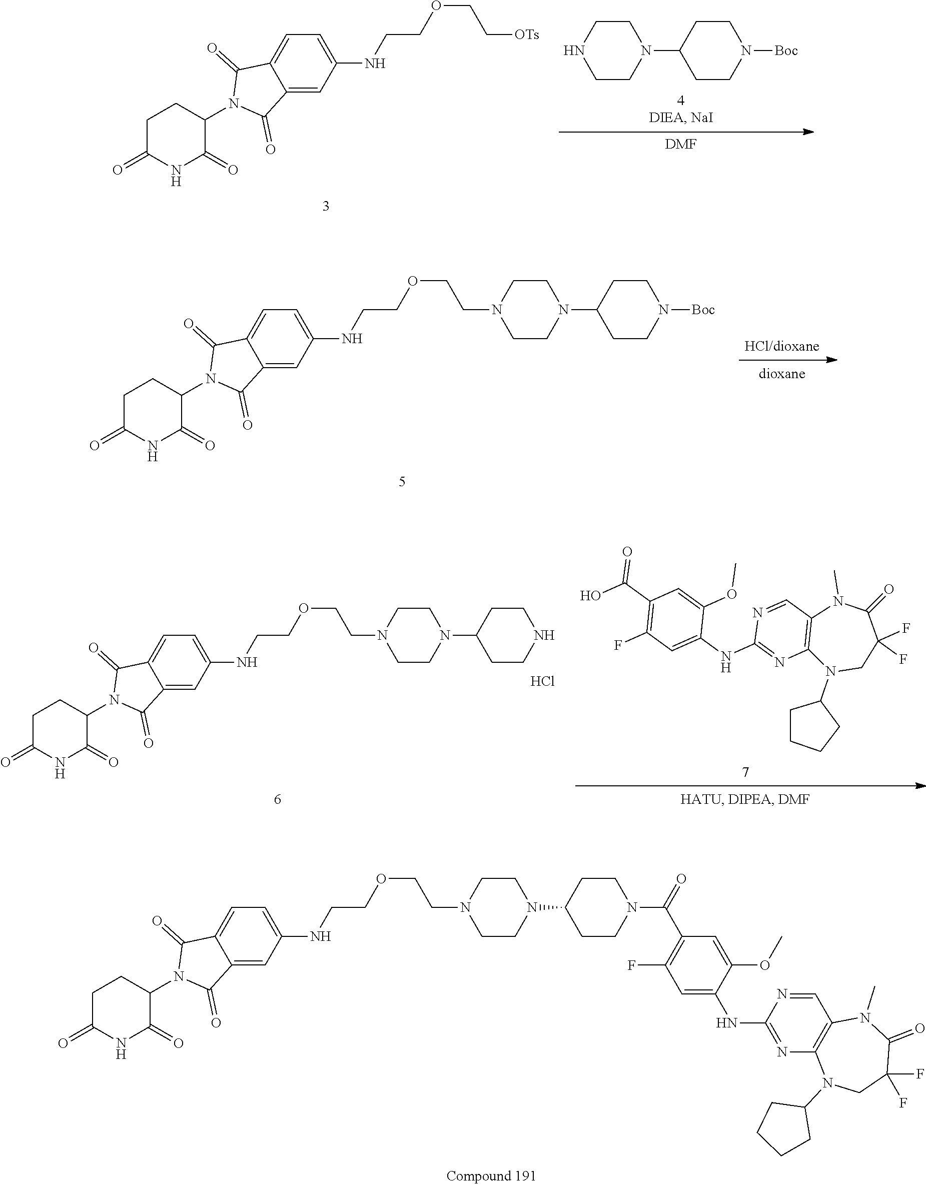

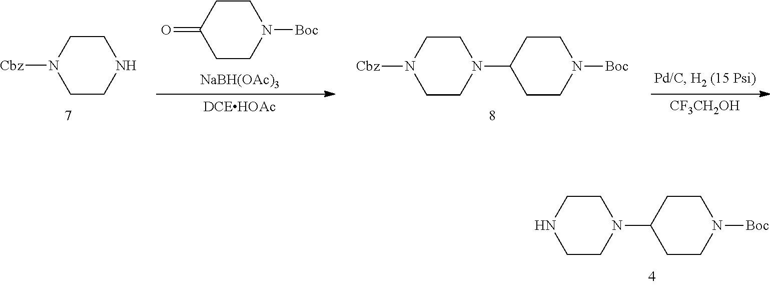

### Step 1. Synthesis of 2-(2,6-dioxopiperidin-3-yl)-5-((2-(2-hydroxyethoxy)ethyl)amino)isoindoline-1,3-dione (2)

To a solution of 2-(2,6-dioxopiperidin-3-yl)-5-fluoroisoindoline-1,3-dione (2 g, 7.24 mmol) in DMSO (10 mL) were added 2-(2-aminoethoxy)ethanol (913.49 mg, 8.69 mmol, 869.99 μL) and TEA (879.21 mg, 8.69 mmol, 1.21 mL) at 25° C. The mixture was stirred at 90° C. for 12 h. LCMS showed a main peak with desired compound mass. The reaction mixture was diluted with water (50 mL) and extracted with EtOAc (50 mL×6). The combined organic layers were washed with saturated brine (100 mL), dried over anhydrous sodium sulfate, filtered and concentrated under reduced pressure to give a residue. The residue was purified by flash silica gel chromatography (ISCO®; 20 g Sepa Flash® Silica Flash Column, Eluent of 0˜100% petroleum ether:EtOAc gradient @80 mL/min) to afford 2-(2,6-dioxopiperidin-3-yl)-5-((2-(2-hydroxyethoxy)ethyl)amino)isoindoline-1,3-dione (0.5 g, 1.38 mmol, 19.11% yield, 100% purity) as yellow oil. MS(M+H)⁺=362.2

### Step 2. Synthesis of benzyl 4-(1-(tert-butoxycarbonyl)piperidin-4-yl)piperazine-1-carboxylate (8)

To a mixture of benzyl piperazine-1-carboxylate (5 g, 22.70 mmol, 4.39 mL) in DCE (50 mL) were added tert-butyl 4-oxopiperidine-1-carboxylate (4.52 g, 22.70 mmol), HOAc (1.43 g, 23.83 mmol, 1.36 mL) at 25° C. Then NaBH(OAc)₃ (19.24 g, 90.80 mmol) was added to the mixture after stirring for 2 h at 25° C. The mixture was stirred at 25° C. for 12 h. LCMS showed ˜70% of desired mass was detected. To the reaction mixture was added H₂O (100 mL) at 0° C., then Na₂CO₃ (sat. aq. 50 mL) was added until pH=10 at 0° C., the combined water layers was extracted with EtOAc (100 mL×3), then the combined organic layers were washed with brine (100 mL×2), dried over anhydrous sodium sulfate, filtered and the filtrate was concentrated under reduced pressure to give a residue. The residue was purified by flash silica gel chromatography (80 g Sepa Flash® Silica Flash Column, eluent of 0˜50% EtOAc/petroleum ether gradient @80 mL/min) to afford benzyl 4-(1-(tert-butoxycarbonyl)piperidin-4-yl)piperazine-1-carboxylate (6.2 g, 15.20 mmol, 66.94% yield, 98.9% purity) as yellow oil. MS(M+H)⁺=404.2

### Step 3. Synthesis of tert-butyl 4-(piperazin-1-yl)piperidine-1-carboxylate (4)

To a mixture of benzyl 4-(1-(tert-butoxycarbonyl)piperidin-4-yl)piperazine-1-carboxylate (3 g, 7.43 mmol) in CF₃CH₂OH (15 mL) was added a mixture of Pd/C (0.5 g, 1.49 mmol, 10 purity) in CF₃CH₂OH (5 mL) at 25° C. under N₂ atmosphere. The mixture was stirred at 25° C. for 2 h under H₂ atmosphere (15 PSI). LCMS showed benzyl 4-(1-(tert-butoxycarbonyl)piperidin-4-yl)piperazine-1-carboxylate consumed. The mixture was filtered to remove the catalyst. The filtrate was concentrated under reduced pressure to afford tert-butyl 4-(piperazin-1-yl)piperidine-1-carboxylate (2.5 g, crude) as yellow oil. MS(M+H)⁺=270.4

¹H NMR (400 MHz, DMSO-d₆) δ=3.94-3.83 (m, 4H), 3.45-3.42 (m, 1H), 2.66-2.63 (m, 4H), 2.37-2.35 (m, 4H), 2.30-2.23 (m, 1H), 1.70-1.67 (m, 2H), 1.38 (s, 9H), 1.24-1.20 (m, 2H).

### Step 4. Synthesis of 2-(2-((2-(2,6-dioxopiperidin-3-yl)-1,3-dioxoisoindolin-5-yl)amino)ethoxy)ethyl 4-methylbenzenesulfonate (3)

To a mixture of 2-(2,6-dioxopiperidin-3-yl)-5-((2-(2-hydroxyethoxy)ethyl)amino)isoindoline-1,3-dione (0.5 g, 1.38 mmol) in DCM (10 mL) were added TosCl (395.70 mg, 2.08 mmol), DMAP (33.81 mg, 276.74 μmol) and TEA (420.05 mg, 4.15 mmol, 577.79 μL) at 25° C. The mixture was stirred at 25° C. for 12 h. LCMS showed ˜78% of desired mass was detected. The reaction mixture was concentrated under reduced pressure to remove DCM. The residue was diluted with water (50 mL) and extracted with EtOAc (50 mL×4). The combined organic layers were washed with saturated brine (100 mL), dried over anhydrous sodium sulfate, filtered and concentrated under reduced pressure to give a residue. The residue was purified by flash silica gel chromatography (ISCO®; 20 g Sepa Flash® Silica Flash Column, Eluent of 0˜70% petroleum ether:EtOAc gradient @60 mL/min) to afford 2-(2-((2-(2,6-dioxopiperidin-3-yl)-1,3-dioxoisoindolin-5-yl)amino)ethoxy)ethyl 4-methylbenzenesulfonate (330 mg, 640.11 μmol, 46.26% yield, 100% purity) as a yellow solid. MS(M+H)⁺=516.2

### Step 5. Synthesis of tert-butyl 4-(4-(2-(2-((2-(2,6-dioxopiperidin-3-yl)-1,3-dioxoisoindolin-5-yl)amino)ethoxy)ethyl)piperazin-1-yl)piperidine-1-carboxylate (5)

To a solution of 2-(2-((2-(2,6-dioxopiperidin-3-yl)-1,3-dioxoisoindolin-5-yl)amino)ethoxy)ethyl 4-methylbenzenesulfonate (330 mg, 640.11 μmol) in DMF (3 mL) were added DIEA (248.19 mg, 1.92 mmol, 334.49 μL), NaI (19.19 mg, 128.02 μmol) and tert-butyl 4-(piperazin-1-yl)piperidine-1-carboxylate (189.68 mg, 704.12 μmol) at 25° C. The mixture was stirred at 70° C. for 12 h. LCMS showed ˜86% of desired mass was detected. The reaction mixture was diluted with water (50 mL) and extracted with EtOAc (50 mL×3). The combined organic layers were washed with saturated brine (50 mL), dried over anhydrous sodium sulfate, filtered and concentrated under reduced pressure to give a residue. The residue was purified by flash silica gel chromatography (ISCO®; 20 g Sepa Flash® Silica Flash Column, Eluent of 0˜60% EtOAc:ethanol gradient @80 mL/min) to afford tert-butyl 4-(4-(2-(2-((2-(2,6-dioxopiperidin-3-yl)-1,3-dioxoisoindolin-5-yl)amino)ethoxy)ethyl) piperazin-1-yl)piperidine-1-carboxylate (0.18 g, 264.40 μmol, 41.30% yield, 90% purity) as yellow oil. MS(M+H)⁺=613.4

### Step 6. Synthesis of 2-(2,6-dioxopiperidin-3-yl)-5-((2-(2-(4-(piperidin-4-yl)piperazin-1-yl)ethoxy)ethyl)amino)isoindoline-1,3-dione (6)

To a solution of tert-butyl 4-(4-(2-(2-((2-(2,6-dioxopiperidin-3-yl)-1,3-dioxoisoindolin-5-yl)amino)ethoxy)ethyl) piperazin-1-yl)piperidine-1-carboxylate (180 mg, 293.77 μmol) in dioxane (2 mL) was added HCl/dioxane (4 M, 2 mL) at 25° C. The mixture was stirred at 25° C. for 1 h. TLC (EtOAc:methanol=2:1) indicated one new spot formed. The mixture was concentrated under reduced pressure to afford 2-(2,6-dioxopiperidin-3-yl)-5-((2-(2-(4-(piperidin-4-yl)piperazin-1-yl)ethoxy)ethyl)amino)isoindoline-1,3-dione (180 mg, 229.48 μmol, 78.12% yield, 70% purity, HCl) as a yellow solid. MS(M+H)⁺=513.4

### Step 7. Synthesis of 5-((2-(2-(4-(1-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoyl)piperidin-4-yl)piperazin-1-yl)ethoxy)ethyl)amino)-2-(2,6-dioxopiperidin-3-yl)isoindoline-1,3-dione (Compound 191)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (60 mg, 128.91 μmol) in DMF (2 mL) was added DIPEA (83.31 mg, 644.57 μmol, 112.27 μL) and HATU (147.05 mg, 386.74 μmol) at 25° C. 2-(2,6-dioxopiperidin-3-yl)-5-((2-(2-(4-(piperidin-4-yl)piperazin-1-yl)ethoxy)ethyl)amino)isoindoline-1,3-dione (106.17 mg, 193.37 μmol, HCl) was added to the mixture after 0.5 h. The mixture was stirred at 25° C. for 12 h. LCMS showed ˜66% of desired mass was detected. The reaction mixture was diluted with water (50 mL) and extracted with EtOAc (50 mL×5). The combined organic layers were washed with brine (100 mL), dried over anhydrous sodium sulfate, filtered and concentrated under reduced pressure to give a residue. The residue was purified by prep-HPLC (column: YMC Triart 30×150 mm×7 μm; mobile phase: [water(HCl)-ACN]; B %: 23%-43%, 9 min) and prep-HPLC (column: Waters Xbridge 150×25 mm×5 μm; mobile phase: [water (NH₄HCO₃)-ACN]; B %: 41%-71%, 10 min) followed by lyophilization, to afford 5 5-((2-(2-(4-(1-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoyl)piperidin-4-yl)piperazin-1-yl)ethoxy)ethyl)amino)-2-(2,6-dioxopiperidin-3-yl)isoindoline-1,3-dione (28 mg, 27.12 μmol, 21.04% yield, 93% purity) as a yellow solid. MS(M+H)⁺=960.4

¹H NMR (400 MHz, DMSO-d₆) δ=11.05 (br s, 1H), 8.27 (s, 1H), 8.14 (d, J=12 Hz, 1H), 8.03 (s, 1H), 7.55 (d, J=8.4 Hz, 1H), 7.15 (t, J=5.2 Hz, 1H), 7.00-6.97 (m, 2H), 6.88 (dd, J=2.0, 8.4 Hz, 1H), 5.02 (dd, J=4.8, 12.4 Hz, 1H), 4.79-4.75 (m, 1H), 4.46-4.43 (m, 1H), 4.05 (t, J=14 Hz, 2H), 3.87 (s, 3H), 3.56 (t, J=5.2 Hz, 2H), 3.51 (t, J=6.0 Hz, 2H), 3.32 (s, 3H), 3.06-2.98 (m, 2H), 2.89-2.74 (m, 4H), 2.49-2.32 (m, 10H), 1.99-1.54 (m, 14H), 1.32-1.28 (m, 2H).

### Example 192. Synthesis of 5-((3-(2-(4-(1-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoyl)piperidin-4-yl)piperazin-1-yl)ethoxy)propyl)amino)-2-(2,6-dioxopiperidin-3-yl)isoindoline-1,3-dione (Compound 192)

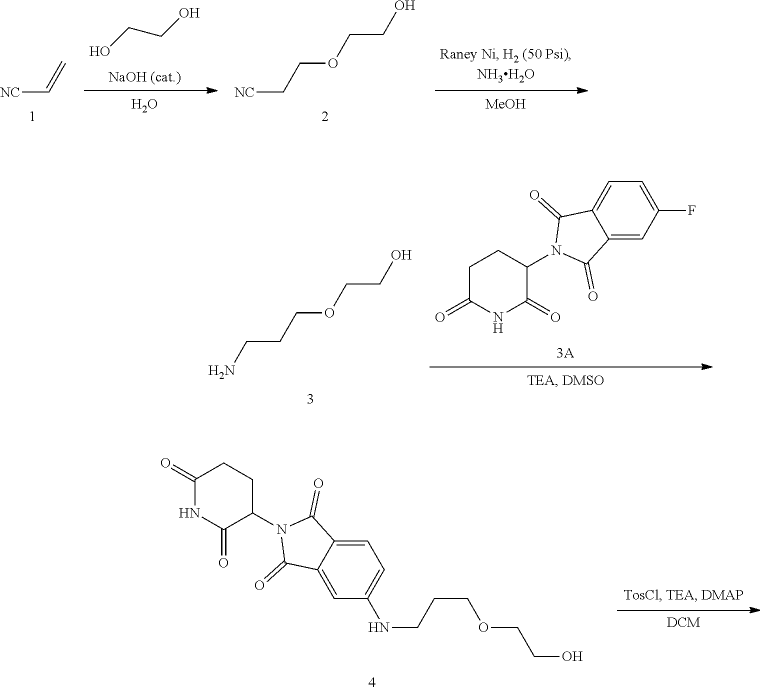

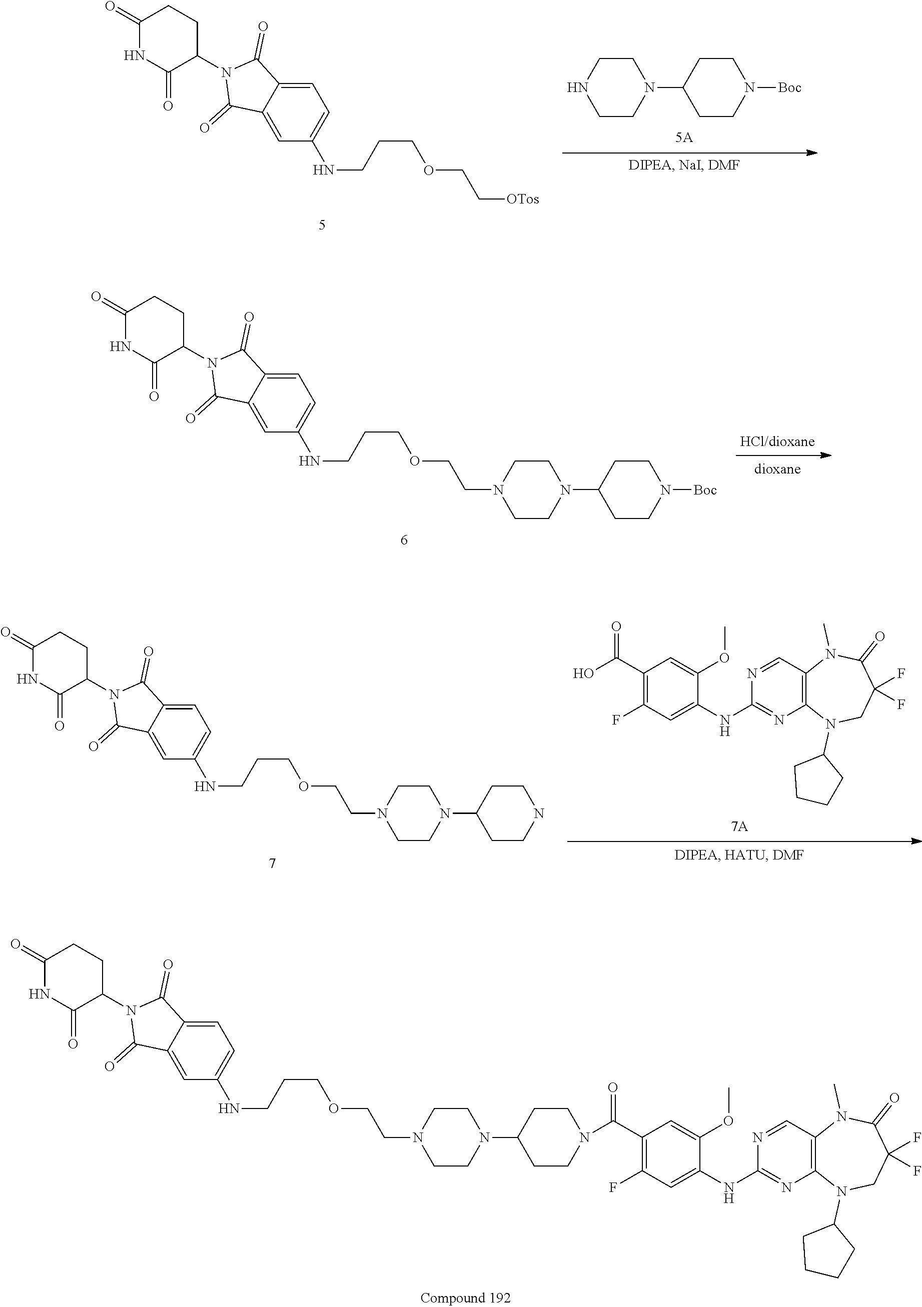

The compound 192 was synthesized by the method described in the scheme similar to the method described in Example 191.

MS(M+H)⁺=974.0, ¹H NMR (400 MHz, DMSO-d₆) δ=11.05 (s, 1H), 8.27 (s, 1H), 8.15 (d, J=12 Hz, 1H), 8.03 (s, 1H), 7.55 (d, J=16 Hz, 1H), 7.10 (t, J=8 Hz, 1H), 6.98 (d, J=6 Hz, 1H), 6.93 (d, J=1.6 Hz, 1H), 6.84 (dd, J=8 Hz, 1.6 Hz, 1H), 5.04-4.99 (m, 1H), 4.81-4.73 (m, 1H), 4.48-4.40 (m, 1H), 4.05 (t, J=14 Hz, 2H), 3.87 (s, 3H), 3.52-3.42 (m, 5H), 3.31-3.29 (m, 2H), 3.24-3.19 (m, 2H), 3.06-2.99 (m, 1H), 2.91-2.74 (m, 2H), 2.60-2.53 (m, 3H), 2.46-2.35 (m, 10H), 2.01-1.83 (m, 4H), 1.83-1.55 (m, 10H), 1.32-1.28 (m, 2H).

### Example 193. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(4-(2-(2-((2-(2,6-dioxopiperidin-3-yl)-1,3-dioxoisoindolin-5-yl)amino)ethoxy)ethyl)piperazin-1-yl)cyclohexyl)-2-fluoro-5-methoxybenzamide (Compound 193)

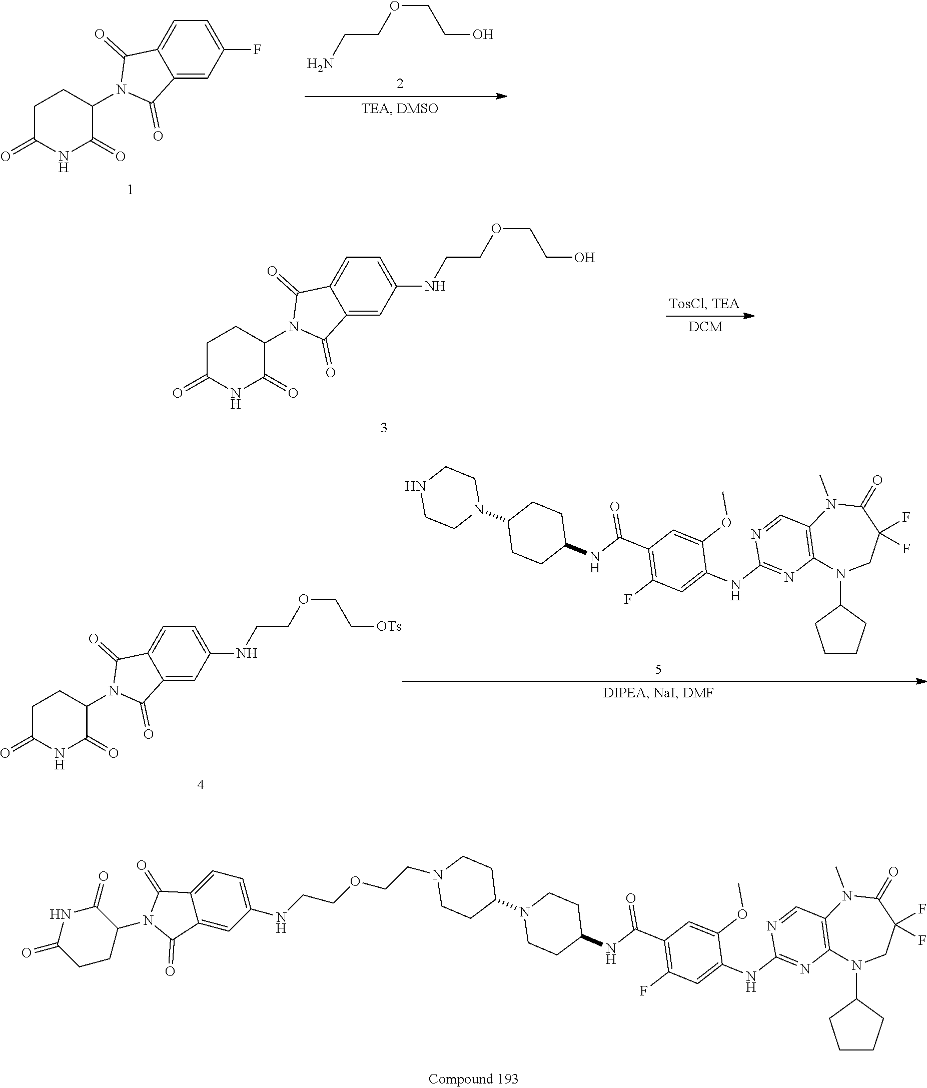

### Step 1. Synthesis of 2-(2,6-dioxopiperidin-3-yl)-5-((2-(2-hydroxyethoxy)ethyl)amino)isoindoline-1,3-dione (3)

To a solution of 2-(2,6-dioxopiperidin-3-yl)-5-fluoroisoindoline-1,3-dione (1.2 g, 4.34 mmol) in DMSO (5 mL) were added TEA (1.32 g, 13.03 mmol, 1.81 mL) and 2-(2-aminoethoxy)ethanol (685.12 mg, 6.52 mmol, 652.49 μL) at 25° C. The mixture was stirred at 90° C. for 12 h. LCMS showed a main peak with desired mass. The mixture was diluted with water (30 mL) and extracted with EtOAc (50 mL×3). The combined organic layers were washed with brine (50 mL), dried over anhydrous sodium sulfate, filtered and concentrated under reduced pressure. The residue was purified by flash silica gel chromatography (ISCO®; 12 g Sepa Flash® Silica Flash Column, Eluent of 0˜100% petroleum ether:EtOAc gradient @40 mL/min) to afford 2-(2,6-dioxopiperidin-3-yl)-5-((2-(2-hydroxyethoxy)ethyl)amino)isoindoline-1,3-dione (0.4 g, 1.11 mmol, 25.48% yield, 100% purity) as yellow oil. MS(M+H)⁺=362.0

### Step 2. Synthesis of 2-(2-((2-(2,6-dioxopiperidin-3-yl)-1,3-dioxoisoindolin-5-yl)amino)ethoxy)ethyl 4-methylbenzenesulfonate (4)

To a solution of 2-(2,6-dioxopiperidin-3-yl)-5-((2-(2-hydroxyethoxy)ethyl)amino)isoindoline-1,3-dione (0.4 g, 1.11 mmol) in DCM (10 mL) were added TosCl (633.12 mg, 3.32 mmol) and TEA (336.04 mg, 3.32 mmol, 462.23 μL) at 25° C. The mixture was stirred at 25° C. for 12 h. LCMS showed a main peak with desired mass. The reaction mixture was concentrated under reduced pressure. The residue was diluted with water (50 mL) and extracted with EtOAc (50 mL×3). The combined organic layers were washed with brine (50 mL), dried over anhydrous sodium sulfate, filtered and concentrated under reduced pressure. The residue was purified by flash silica gel chromatography (ISCO®; 20 g SepaFlash® Silica Flash Column, Eluent of 0˜80% petroleum ether:EtOAc gradient @60 mL/min) to afford 2-(2-((2-(2,6-dioxopiperidin-3-yl)-1,3-dioxoisoindolin-5-yl)amino)ethoxy)ethyl 4-methylbenzenesulfonate (270 mg, 466.12 μmol, 42.11% yield, 89% purity) as yellow oil. MS(M+H)⁺=516.1

### Step 3. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(4-(2-(2-((2-(2,6-dioxopiperidin-3-yl)-1,3-dioxoisoindolin-5-yl)amino)ethoxy)ethyl)piperazin-1-yl)cyclohexyl)-2-fluoro-5-methoxybenzamide (Compound 193)

To a solution of 2-(2-((2-(2,6-dioxopiperidin-3-yl)-1,3-dioxoisoindolin-5-yl)amino)ethoxy)ethyl 4-methylbenzenesulfonate (150 mg, 290.96 μmol) in DMF (2 mL) were added NaI (8.72 mg, 58.19 μmol), 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxy-N-((1r,4r)-4-(piperazin-1-yl)cyclohexyl) benzamide (201.86 mg, 320.06 μmol) and DIPEA (112.81 mg, 872.88 μmol, 152.04 μL) at 25° C. The mixture was stirred at 70° C. for 12 h. LCMS showed ˜70% of desired mass was detected. The reaction mixture was diluted with water (30 mL) and extracted with EtOAc (30 mL×4). The combined organic layers were washed with brine (50 mL), dried over anhydrous sodium sulfate, filtered and concentrated under reduced pressure. The residue was purified by prep-HPLC (column: 3_Phenomenex Luna C18 75×30 mm×3 μm; mobile phase: [water(HCl)-ACN]; B %: 26%-46%, 7 min) followed by prep-HPLC (column: Waters Xbridge 150×25 mm×5 μm; mobile phase: [water(NH₄HCO₃)-ACN]; B %: 42%-72%, 9 min) followed by lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(4-(2-(2-((2-(2,6-dioxopiperidin-3-yl)-1,3-dioxoisoindolin-5-yl)amino)ethoxy)ethyl)piperazin-1-yl)cyclohexyl)-2-fluoro-5-methoxybenzamide (56.2 mg, 57.47 μmol, 19.75% yield, 99.6% purity) as a yellow solid. MS (M+H)⁺=974.5.

¹H NMR (400 MHz, DMSO-d₆) δ=11.05 (s, 1H), 8.29 (s, 1H), 8.23 (d, J=9.2 Hz, 1H), 8.02 (s, 1H), 7.82-7.79 (m, 1H), 7.56 (d, J=8.0 Hz, 1H), 7.17 (d, J=6.8 Hz, 1H), 7.14 (t, J=5.6 Hz, 1H), 7.01-7.00 (m, 1H), 6.90-6.88 (m, 1H), 5.06-5.01 (m, 1H), 4.84-4.79 (m, 1H), 4.07 (t, J=14 Hz, 2H), 3.90 (s, 3H), 3.68-3.65 (m, 1H), 3.56 (t, J=5.2 Hz, 2H), 3.52 (t, J=6.0 Hz, 2H), 3.39-3.36 (m, 2H), 3.30 (s, 3H), 2.91-2.83 (m, 1H), 2.59-2.53 (m, 1H), 2.45-2.12 (m, 12H), 2.01-1.58 (m, 13H), 1.33-1.23 (m, 4H).

### Example 194. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(4-(2-(2-((2-(2,6-dioxopiperidin-3-yl)-1,3-dioxoisoindolin-4-yl)amino)ethoxy)ethyl)piperazin-1-yl)cyclohexyl)-3-methoxybenzamide (Compound 194)

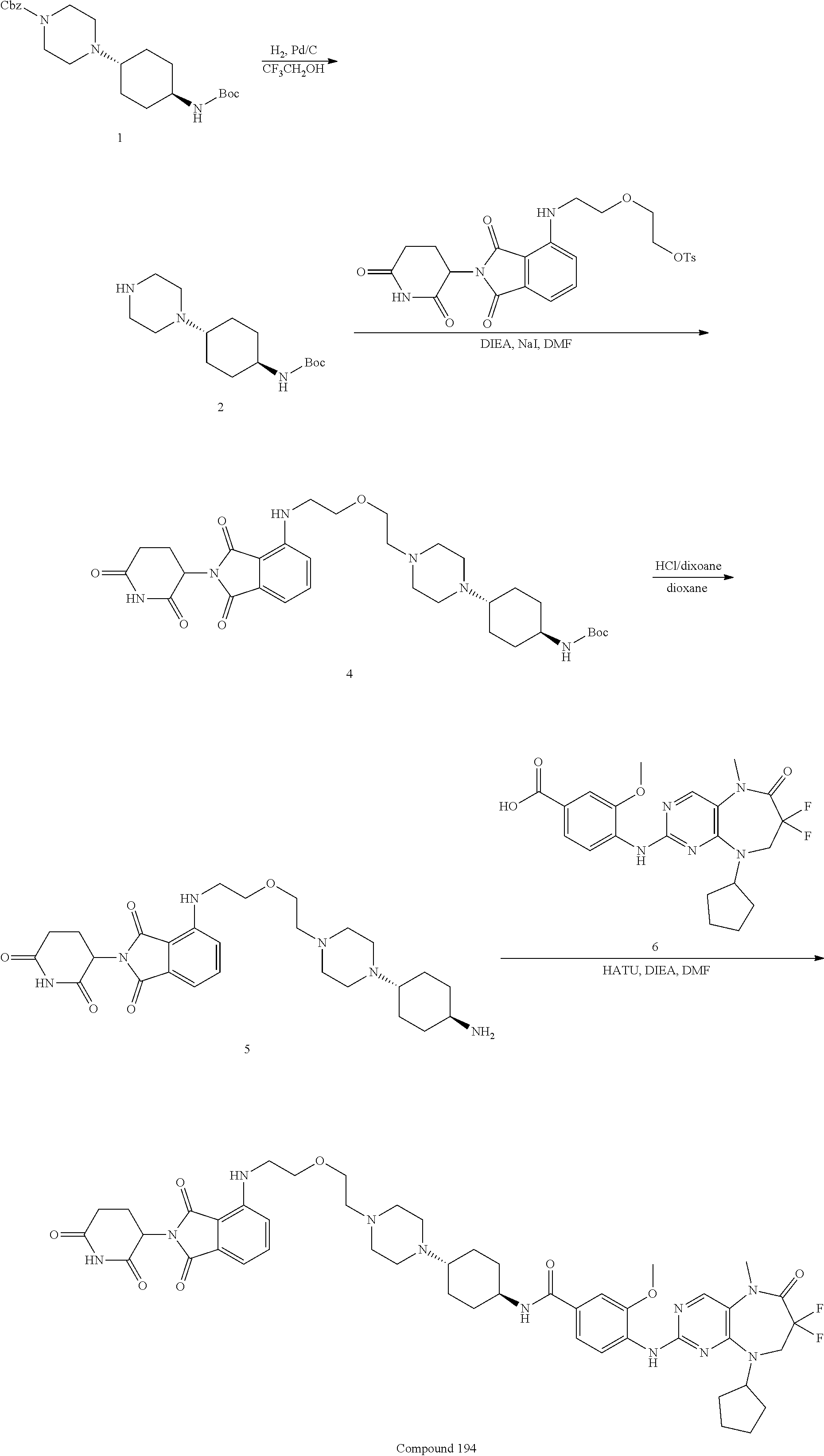

The compound 194 was synthesized by the method described in the scheme similar to the method described in Example 191.

MS(M+H)⁺=956.4, ¹H NMR (400 MHz, DMSO-d₆) δ=11.09 (s, 1H), 8.27-8.25 (m, 2H), 8.05 (d, J=8.0 Hz, 1H), 7.95 (s, 1H), 7.58 (t, J=8.0 Hz, 1H), 7.48-7.46 (m, 2H), 7.15 (d, J=8.4 Hz, 1H), 7.04 (d, J=7.2 Hz, 1H), 6.59 (t, J=5.6 Hz, 1H), 5.08-5.03 (m, 1H), 4.78-4.74 (m, 1H), 4.04 (t, J=14 Hz, 2H), 3.93 (s, 3H), 3.71-3.68 (m, 1H), 3.59 (t, J=5.2 Hz, 2H), 3.53 (t, J=5.6 Hz, 2H), 3.48-3.44 (m, 2H), 3.33 (s, 3H), 2.94-2.85 (m, 1H), 2.61-2.55 (m, 1H), 2.45-2.38 (m, 10H), 2.19-2.14 (m, 2H), 2.04-1.58 (m, 13H), 1.40-1.21 (m, 4H).

### Example 195. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(4-(2-(2-((2-(2,6-dioxopiperidin-3-yl)-1,3-dioxoisoindolin-4-yl)amino)ethoxy)acetyl)piperazin-1-yl)cyclohexyl)-2-fluoro-5-methoxybenzamide (Compound 195)

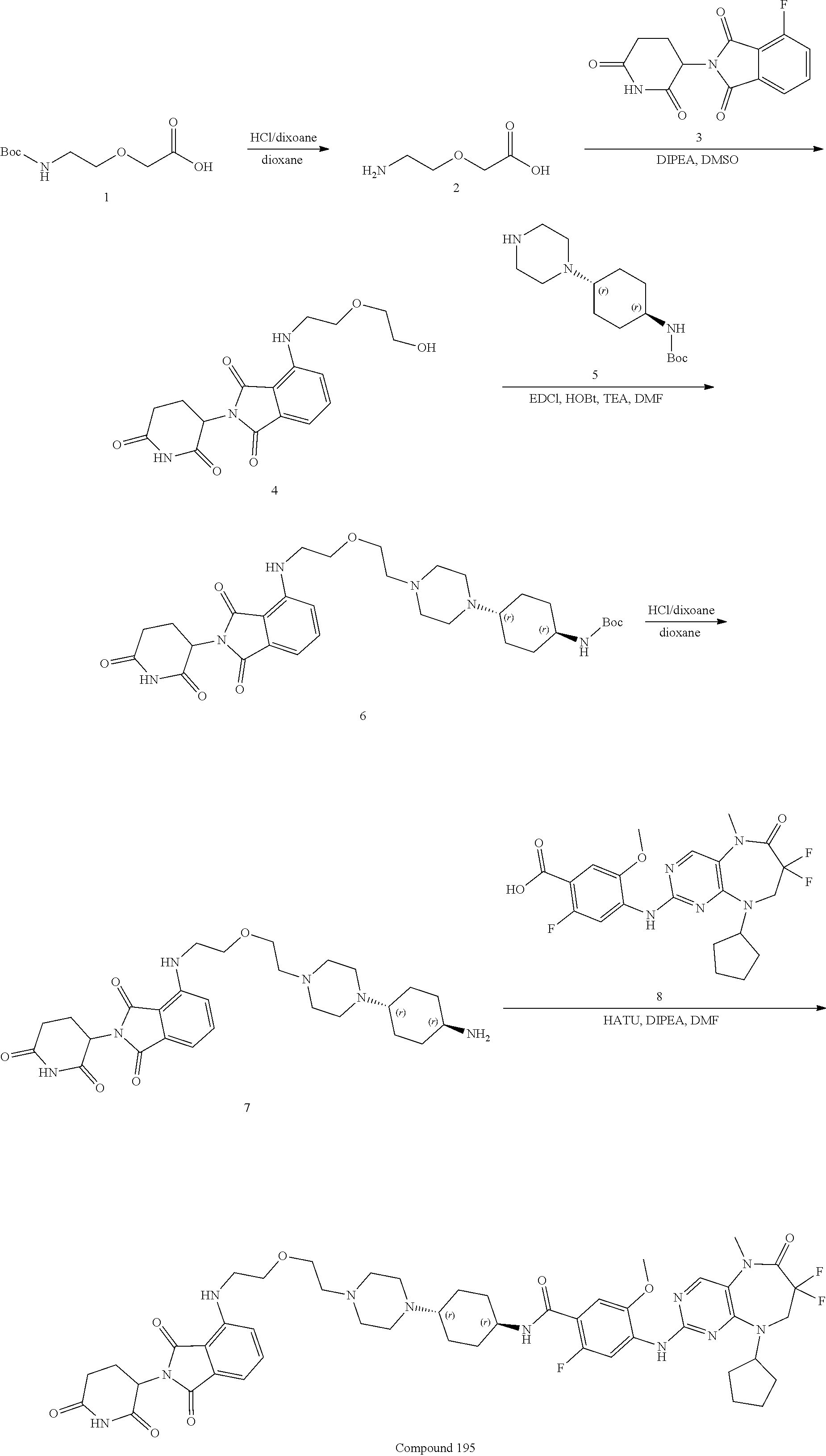

### Step 1. Synthesis of 2-(2-aminoethoxy)acetic acid (2)

To a solution of 2-(2-((tert-butoxycarbonyl)amino)ethoxy)acetic acid (0.7 g, 3.19 mmol) in dioxane (10 mL) was added HCl/dioxane (4 M, 14.00 mL, 17.54 eq) at 20° C. and the resulting mixture was stirred at 20° C. for 1 h. TLC (SiO₂, Dichloromethane:Methanol=10:1) indicated starting material was consumed completely and one new spot was detected. The reaction mixture was concentrated in vacuum to afford 2-(2-aminoethoxy)acetic acid (0.51 g, crude, HCl) as a colorless oil. MS(M+H)⁺=120.1

### Step 2. Synthesis of 2-(2-((2-(2,6-dioxopiperidin-3-yl)-1,3-dioxoisoindolin-4-yl)amino)ethoxy)acetic acid (4)

To a solution of 2-(2,6-dioxo-3-piperidyl)-4-fluoro-isoindoline-1,3-dione (800 mg, 2.90 mmol) in DMSO (15 mL) were added DIPEA (1.12 g, 8.69 mmol, 1.51 mL) and 2-(2-aminoethoxy) acetic acid (495.66 mg, 3.19 mmol, HCl) at 20° C. and the resulting mixture was stirred at 130° C. for 16 h. LCMS showed 55% of 2-(2,6-dioxo-3-piperidyl)-4-fluoro-isoindoline-1,3-dione remained and 18% peak with desired mass was detected. The reaction mixture was diluted with H₂O (10 mL) and extracted with EtOAc (10 mL×3). The aqueous phase was concentrated in vacuum to afford crude product (1.7 g, 43% purity). The crude product was purified by prep-HPLC (column: 3_Phenomenex Luna C18 75*30 mm*3 um; mobile phase: [water (HCl)-ACN]; B %: 13%-43%, 7 min) and lyophilization to afford 2-(2-((2-(2,6-dioxopiperidin-3-yl)-1,3-dioxoisoindolin-4-yl)amino)ethoxy)acetic acid (162 mg, 431.62 μmol, 14.90% yield) as a yellow solid. MS(M+H)⁺=376.3

### Step 3. Synthesis of tert-butyl ((1r,4r)-4-(4-(2-(2-((2-(2,6-dioxopiperidin-3-yl)-1,3-dioxoisoindolin-4-yl)amino)ethoxy)acetyl)piperazin-1-yl)cyclohexyl)carbamate (6)

To a solution of 2-(2-((2-(2,6-dioxopiperidin-3-yl)-1,3-dioxoisoindolin-4-yl)amino)ethoxy)acetic acid (162 mg, 431.62 μmol) and tert-butyl N-(4-piperazin-1-ylcyclohexyl) carbamate (134.56 mg, 474.78 μmol) in DMF (6 mL) were added HOBt (87.48 mg, 647.43 μmol), EDCI (124.11 mg, 647.43 μmol) and TEA (131.02 mg, 1.29 mmol, 180.23 μL) at 20° C. and the resulting mixture was stirred at 20° C. for 12 h. LCMS showed all starting material was consumed completely and 77% peak with desired mass was detected. The reaction mixture was diluted with H₂O (12 mL) and extracted with EtOAc (12 mL×3). The organic layer was washed with brine (12 mL×3), dried over Na₂SO₄, filtered and concentrated. The residue was purified by flash silica gel chromatography (10 g SepaFlash® Silica Flash Column, Eluent of 0˜100% EtOAc/Petroleum ether to 0˜10% Dichloromethane/Methanol gradient @100 mL/min) to afford tert-butyl ((1r,4r)-4-(4-(2-(2-((2-(2,6-dioxopiperidin-3-yl)-1,3-dioxoisoindolin-4-yl)amino)ethoxy)acetyl)piperazin-1-yl)cyclohexyl)carbamate (180 mg, 275.31 μmol, 63.79% yield, 98% purity) as a yellow oil. MS(M+H)⁺=641.2

### Step 4. Synthesis of 4-((2-(2-(4-((1r,4r)-4-aminocyclohexyl)piperazin-1-yl)-2-oxoethoxy)ethyl)amino)-2-(2,6-dioxopiperidin-3-yl)isoindoline-1,3-dione (7)

To a solution of tert-butyl ((1r,4r)-4-(4-(2-(2-((2-(2,6-dioxopiperidin-3-yl)-1,3-dioxoisoindolin-4-yl)amino)ethoxy)acetyl)piperazin-1-yl)cyclohexyl)carbamate (180 mg, 280.93 μmol) in dioxane (2 mL) was added HCl/dioxane (4 M, 10 mL) at 20° C. and the resulting mixture was stirred at 20° C. for 0.5 h. LCMS showed starting material was consumed completely and 85% peak with desired mass was detected. The reaction mixture was concentrated in vacuum to afford 4-((2-(2-(4-((1r,4r)-4-aminocyclohexyl)piperazin-1-yl)-2-oxoethoxy)ethyl)amino)-2-(2,6-dioxopiperidin-3-yl)isoindoline-1,3-dione (165 mg, crude, HCl) as a yellow solid. MS(M+H)⁺=541.1

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(4-(2-(2-((2-(2,6-dioxopiperidin-3-yl)-1,3-dioxoisoindolin-4-yl)amino)ethoxy)acetyl)piperazin-1-yl)cyclohexyl)-2-fluoro-5-methoxybenzamide (Compound 195)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (80 mg, 171.89 μmol) in DMF (2 mL) were added HATU (71.89 mg, 189.07 μmol) and DIPEA (44.43 mg, 343.77 μmol, 59.88 μL). The mixture was stirred at 20° C. for 10 min and a solution of 4-((2-(2-(4-((1r,4r)-4-aminocyclohexyl)piperazin-1-yl)-2-oxoethoxy)ethyl)amino)-2-(2,6-dioxopiperidin-3-yl)isoindoline-1,3-dione (109.11 mg, 189.07 μmol, HCl) in DMF (2 mL) and DIPEA (44.43 mg, 343.77 μmol, 59.88 μL) was added and the resulting mixture was stirred at 20° C. for 1 h. LCMS showed all starting material was consumed completely and 61% peak with desired mass was detected. The reaction mixture was diluted with H₂O (12 mL) and extracted with EtOAc (12 mL×3). The organic layer was washed with brine (12 mL×3), dried over Na₂SO₄, filtered and concentrated. The residue was purified by prep-HPLC (column: Phenomenex Synergi C18 150*25 mm*10 um; mobile phase: [water(FA)-ACN]; B %: 20%-50%, 10 min) and re-purified by prep-HPLC (column: Waters Xbridge 150*25 mm*5 um; mobile phase: [water(NH₄HCO₃)-ACN]; B %: 41%-71%, 8 min) and lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(4-(2-(2-((2-(2,6-dioxopiperidin-3-yl)-1,3-dioxoisoindolin-4-yl)amino)ethoxy)acetyl)piperazin-1-yl)cyclohexyl)-2-fluoro-5-methoxybenzamide (24.7 mg, 22.75 μmol, 13.24% yield, 91% purity) as a yellow solid. MS(M+H)⁺=987.5

¹H NMR (400 MHz, DMSO-d₆) δ=11.10 (s, 1H), 8.29 (s, 1H), 8.23 (d, J=13.3 Hz, 1H), 8.03 (s, 1H), 7.81 (dd, J=3.5, 7.8 Hz, 1H), 7.62-7.55 (m, 1H), 7.21-7.12 (m, 2H), 7.05 (d, J=7.1 Hz, 1H), 6.66 (t, J=5.7 Hz, 1H), 5.06 (dd, J=5.4, 13.0 Hz, 1H), 4.87-4.76 (m, 1H), 4.18 (s, 2H), 4.12-4.02 (m, 2H), 3.91 (s, 3H), 3.73-3.62 (m, 3H), 3.57-3.43 (m, 6H), 3.33 (s, 3H), 2.96-2.84 (m, 1H), 2.63-2.54 (m, 2H), 2.44-2.38 (m, 4H), 2.30-2.18 (m, 1H), 2.07-1.93 (m, 3H), 1.93-1.87 (m, 2H), 1.78-1.70 (m, 4H), 1.68-1.56 (m, 4H), 1.39-1.19 (m, 4H).

### Example 196. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(4-(2-((2-(2,6-dioxopiperidin-3-yl)-1,3-dioxoisoindolin-4-yl)amino)ethyl)piperazin-1-yl)cyclohexyl)-2-fluoro-5-methoxybenzamide (Compound 196)

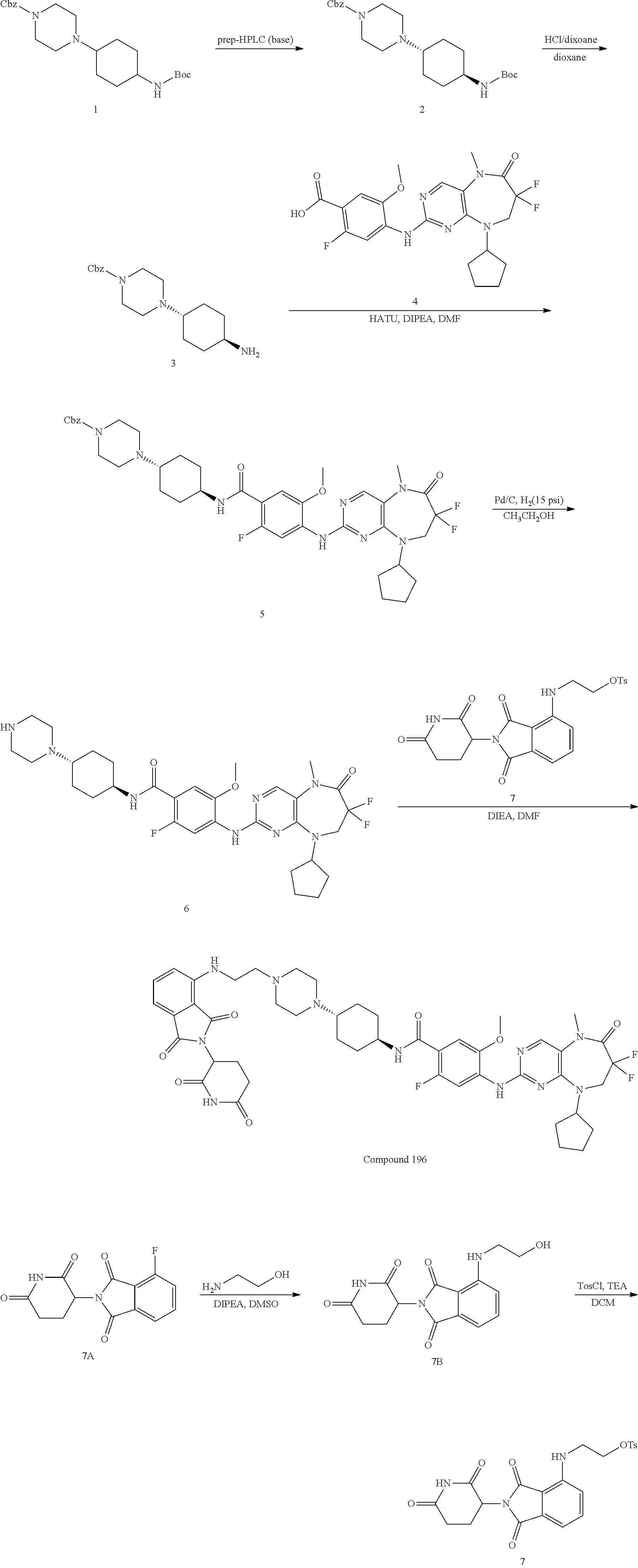

### Step 1. Synthesis of benzyl 4-((1r,4r)-4-((tert-butoxycarbonyl)amino)cyclohexyl)piperazine-1-carboxylate (2)

Benzyl 4-(4-((tert-butoxycarbonyl)amino)cyclohexyl)piperazine-1-carboxylate (11 g) was purified by prep-HPLC (column: Kromasil Eternity XT 250*80 mm*10 um; mobile phase: [water(ammonia hydroxide v/v)-ACN]; B %: 40%-70%, 20 min) and the eluent was lyophilized to afford benzyl 4-((1r,4r)-4-((tert-butoxycarbonyl)amino)cyclohexyl)piperazine-1-carboxylate (5.4 g, 12.67 mmol, 66.15% yield, 98% purity) as a white solid. MS(M+H)⁺=418.6,

SFC (retention time: peak 1=1.589 min; method: Column: Chiralpak AD-3 50×4.6 mm I. D., 3 um; Mobile phase: Phase A for CO₂, and Phase B for IPA (0.05% DEA); Gradient elution: B in A from 5% to 40%; Flow rate: 3 mL/min; Detector: DAD; Column Temp: 35° C.; Back Pressure: 100 Bar).

### Step 2. Synthesis of benzyl 4-((1r,4r)-4-aminocyclohexyl)piperazine-1-carboxylate (3)

To a solution of benzyl 4-((1r,4r)-4-((tert-butoxycarbonyl)amino)cyclohexyl)piperazine-1-carboxylate (1 g, 2.39 mmol) in dioxane (10 mL) was added HCl/dioxane (4 M, 20 mL, 33.40 eq) at 20° C. and the resulting mixture was stirred at 20° C. for 0.5 h. LCMS showed starting material was consumed completely and a peak with desired mass. The reaction mixture was concentrated in vacuo to afford benzyl 4-((1r,4r)-4-aminocyclohexyl)piperazine-1-carboxylate (1 g, crude, HCl) as a white solid. MS(M+H)⁺=318.2

### Step 3. Synthesis of benzyl 4-((1r,4r)-4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzamido)cyclohexyl)piperazine-1-carboxylate (5)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (1 g, 2.15 mmol) in DMF (10 mL) were added HATU (898.65 mg, 2.36 mmol) and DIPEA (555.38 mg, 4.30 mmol, 748.49 μL). The mixture was stirred at 20° C. for 10 min and a solution of benzyl 4-((1r,4r)-4-aminocyclohexyl)piperazine-1-carboxylate (836.39 mg, 2.36 mmol, HCl) in DMF (10 mL) with DIPEA (555.38 mg, 4.30 mmol, 748.49 μL) were added at 20° C. and the resulting mixture was stirred at 20° C. for 1 h. LCMS showed starting material was consumed completely and a peak (84%) with desired mass. The reaction mixture was diluted with H₂O (20 mL) and extracted with EtOAc (20 mL×3). The organic layer was washed with brine (20 mL×3), dried over Na₂SO₄, filtered and concentrated. The residue was purified by flash silica gel chromatography (25 g SepaFlash® Silica Flash Column, Eluent of 0˜100% EtOAc/Petroleum ether gradient @100 mL/min) to afford benzyl 4-((1r,4r)-4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzamido)cyclohexyl)piperazine-1-carboxylate (1.3 g, 1.65 mmol, 76.74% yield, 97% purity) as an orange solid. MS(M+H)⁺=765.2

### Step 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxy-N-((1r,4r)-4-(piperazin-1-yl)cyclohexyl)benzamide (6)

To a solution of benzyl 4-((1r,4r)-4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzamido)cyclohexyl)piperazine-1-carboxylate (1.3 g, 1.70 mmol) in CF₃CH₂OH (20 mL) was added Pd/C (300 mg, 169.97 μmol, 10% purity) under N₂ atmosphere. The suspension was degassed and purged with H₂ for 3 times. The mixture was stirred under H₂ (15 Psi) at 20° C. for 12 h. LCMS showed starting material was consumed completely and a peak (93%) with desired mass. The reaction mixture was diluted with CF₃CH₂OH (60 mL) and filtered. The filtrate was concentrated in vacuo to afford 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxy-N-((1r,4r)-4-(piperazin-1-yl)cyclohexyl) benzamide (1.1 g, crude) as a yellow oil. MS(M+H)⁺=631.2

### Step 5. Synthesis of 2-(2,6-dioxopiperidin-3-yl)-4-((2-hydroxyethyl)amino)isoindoline-1,3-dione (7B)

To a solution of 2-(2,6-dioxo-3-piperidyl)-4-fluoro-isoindoline-1,3-dione (0.5 g, 1.81 mmol) in DMSO (10 mL) were added DIPEA (701.85 mg, 5.43 mmol, 945.89 μL) and 2-aminoethanol (132.68 mg, 2.17 mmol, 131.37 μL) at 20° C. and the resulting mixture was stirred at 130° C. for 12 h. LCMS showed 2-(2,6-dioxo-3-piperidyl)-4-fluoro-isoindoline-1,3-dione was consumed completely and a peak (95%) with desired mass. The reaction mixture was diluted with H₂O (15 mL) and extracted with EtOAc (15 mL×3). The organic layer was dried over Na₂SO₄, filtered and concentrated. The residue was purified by flash silica gel chromatography (10 g SepaFlash® Silica Flash Column, Eluent of 0˜100% EtOAc/Petroleum ether gradient @100 mL/min) to afford 2-(2,6-dioxopiperidin-3-yl)-4-((2-hydroxyethyl)amino)isoindoline-1,3-dione (569 mg, 1.79 mmol, 99.07% yield) as a yellow oil. MS(M+H)⁺=318.4

### Step 6. Synthesis of 2-((2-(2,6-dioxopiperidin-3-yl)-1,3-dioxoisoindolin-4-yl)amino)ethyl 4-methylbenzenesulfonate (7)

To a solution of 2-(2,6-dioxopiperidin-3-yl)-4-((2-hydroxyethyl)amino)isoindoline-1,3-dione (569 mg, 1.79 mmol) in DCM (10 mL) were added TEA (544.38 mg, 5.38 mmol, 748.80 μL) and TosCl (854.71 mg, 4.48 mmol) at 20° C. and the resulting mixture was stirred at 20° C. for 16 h. LCMS showed 2-(2,6-dioxo-3-piperidyl)-4-(2-hydroxyethylamino) isoindoline-1,3-dione was consumed completely and a peak (27%) with desired mass. The reaction mixture was concentrated in vacuum. The residue was purified by flash silica gel chromatography (10 g SepaFlash® Silica Flash Column, Eluent of 0˜100% EtOAc/Petroleum ether gradient @100 mL/min). The crude product was re-purified by prep-HPLC (column: Phenomenex luna C18 150*40 mm*15 um; mobile phase: [water(FA)-ACN]; B %: 32%-62%, 10 min) and the eluent was lyophilized to afford 2-((2-(2,6-dioxopiperidin-3-yl)-1,3-dioxoisoindolin-4-yl)amino)ethyl 4-methylbenzenesulfonate (117 mg, 248.15 μmol, 13.84% yield, 100% purity) as a yellow solid. MS(M+H)⁺=472.0

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(4-(2-((2-(2,6-dioxopiperidin-3-yl)-1,3-dioxoisoindolin-4-yl)amino)ethyl)piperazin-1-yl)cyclohexyl)-2-fluoro-5-methoxybenzamide (Compound 196)

To a solution of 2-((2-(2,6-dioxopiperidin-3-yl)-1,3-dioxoisoindolin-4-yl)amino)ethyl 4-methylbenzenesulfonate (115 mg, 243.91 μmol) and 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxy-N-((1r,4r)-4-(piperazin-1-yl)cyclohexyl) benzamide (153.84 mg, 243.91 μmol) in DMF (4 mL) were added NaI (7.31 mg, 48.78 μmol) and DIPEA (94.57 mg, 731.73 μmol, 127.46 μL) at 20° C. and the resulting mixture was stirred at 60° C. for 32 h. LCMS showed the starting material was consumed completely and a peak (68%) with desired mass. The reaction mixture was diluted with H₂O (10 mL) and extracted with EtOAc (10 mL×3). The organic layer was dried over Na₂SO₄, filtered and concentrated. The residue was purified by prep-HPLC (column: Unisil 3-100 C18 μLtra 150*50 mm*3 um; mobile phase: [water (FA)-ACN]; B %: 18%-48%, 10 min) and the eluent was lyophilized to afford product A (106.6 mg). The product A (106.6 mg) was re-purified by prep-HPLC (column: Waters Xbridge BEH C18 150*25 mm*5 um; mobile phase: [water(NH₄HCO₃)-ACN]; B %: 49%-79%, 9 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(4-(2-((2-(2,6-dioxopiperidin-3-yl)-1,3-dioxoisoindolin-4-yl)amino)ethyl)piperazin-1-yl)cyclohexyl)-2-fluoro-5-methoxybenzamide (47.6 mg, 46.58 μmol, 19.10% yield, 91% purity) as a yellow solid. MS(M+H)⁺=929.9

¹H NMR (400 MHz, DMSO-d₆) δ=11.09 (s, 1H), 8.30 (s, 1H), 8.24 (d, J=13.2 Hz, 1H), 8.03 (s, 1H), 7.82 (br dd, J=3.4, 7.8 Hz, 1H), 7.59 (t, J=7.8 Hz, 1H), 7.18 (d, J=6.7 Hz, 1H), 7.09 (d, J=8.6 Hz, 1H), 7.03 (d, J=7.0 Hz, 1H), 6.74 (br s, 1H), 5.07 (dd, J=5.3, 12.9 Hz, 1H), 4.82 (quin, J=8.1 Hz, 1H), 4.08 (br t, J=13.9 Hz, 2H), 3.91 (s, 3H), 3.75-3.62 (m, 1H), 3.38-3.35 (m, 2H), 3.32 (s, 3H), 2.96-2.81 (m, 1H), 2.63-2.53 (m, 6H), 2.46-2.34 (m, 4H), 2.24-2.20 (m, 1H), 2.08-1.54 (m, 15H), 1.40-1.24 (m, 4H).

### Example 197. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(4-(3-((2-(2,6-dioxopiperidin-3-yl)-1,3-dioxoisoindolin-4-yl)amino)propyl)piperazin-1-yl)cyclohexyl)-2-fluoro-5-methoxybenzamide (Compound 197)

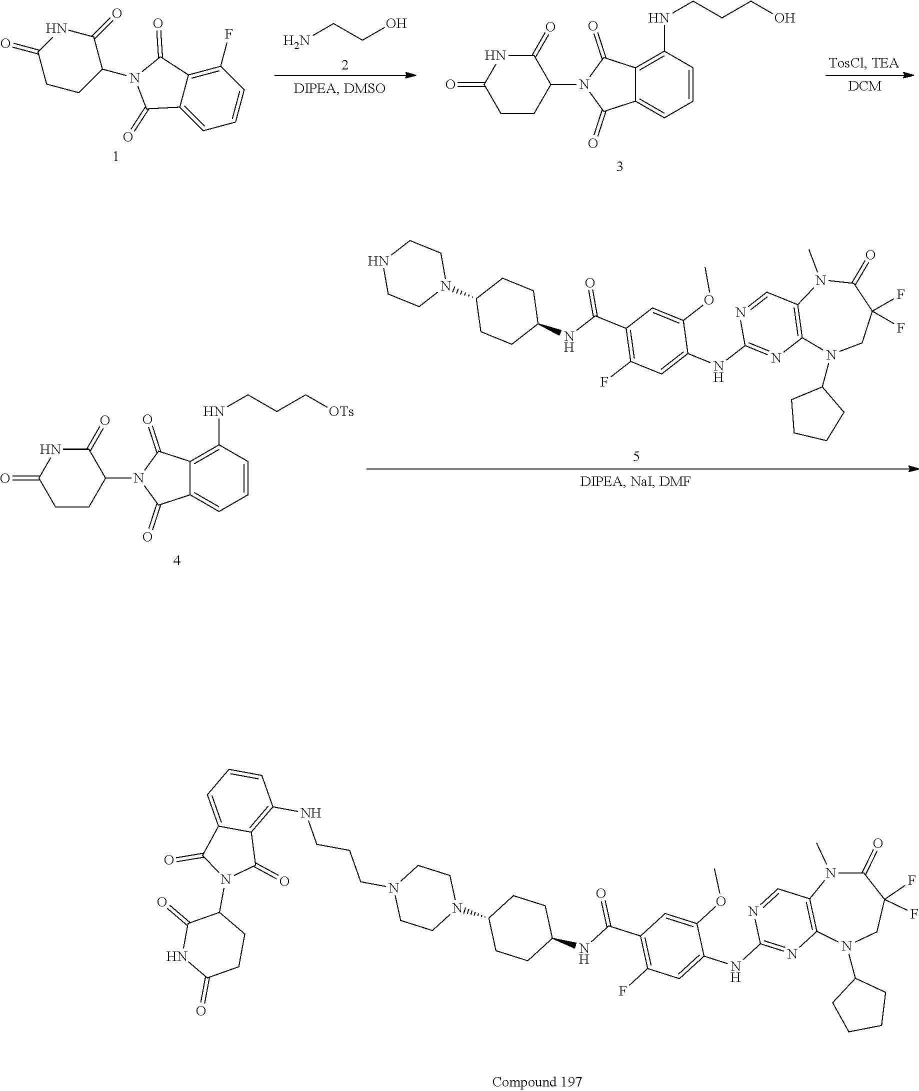

The compound 197 was synthesized by the method described in the scheme similar to the method described in Example 196.

MS(M+H)⁺=943.9, ¹H NMR (400 MHz, DMSO-d₆) δ=11.10 (s, 1H), 8.30 (s, 1H), 8.24 (d, J=13.3 Hz, 1H), 8.03 (s, 1H), 7.81 (br dd, J=3.5, 7.5 Hz, 1H), 7.61-7.54 (m, 1H), 7.19 (d, J=6.7 Hz, 1H), 7.12 (d, J=8.6 Hz, 1H), 7.02 (d, J=7.0 Hz, 1H), 6.77 (br t, J=5.8 Hz, 1H), 5.05 (dd, J=5.3, 12.9 Hz, 1H), 4.87-4.77 (m, 1H), 4.08 (br t, J=13.9 Hz, 2H), 3.91 (s, 3H), 3.74-3.62 (m, 1H), 3.32 (s, 5H), 2.95-2.83 (m, 1H), 2.64-2.53 (m, 6H), 2.38-2.32 (m, 4H), 2.21 (m, 1H), 2.07-1.53 (m, 17H), 1.39-1.23 (m, 4H).

### Example 198. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-((1r,4r)-4-(4-(2-(2-((2-(2,6-dioxopiperidin-3-yl)-1-oxoisoindolin-4-yl)oxy)ethoxy)ethyl)piperazin-1-yl)cyclohexyl)-3-methoxybenzamide (trans) (Compound 198)

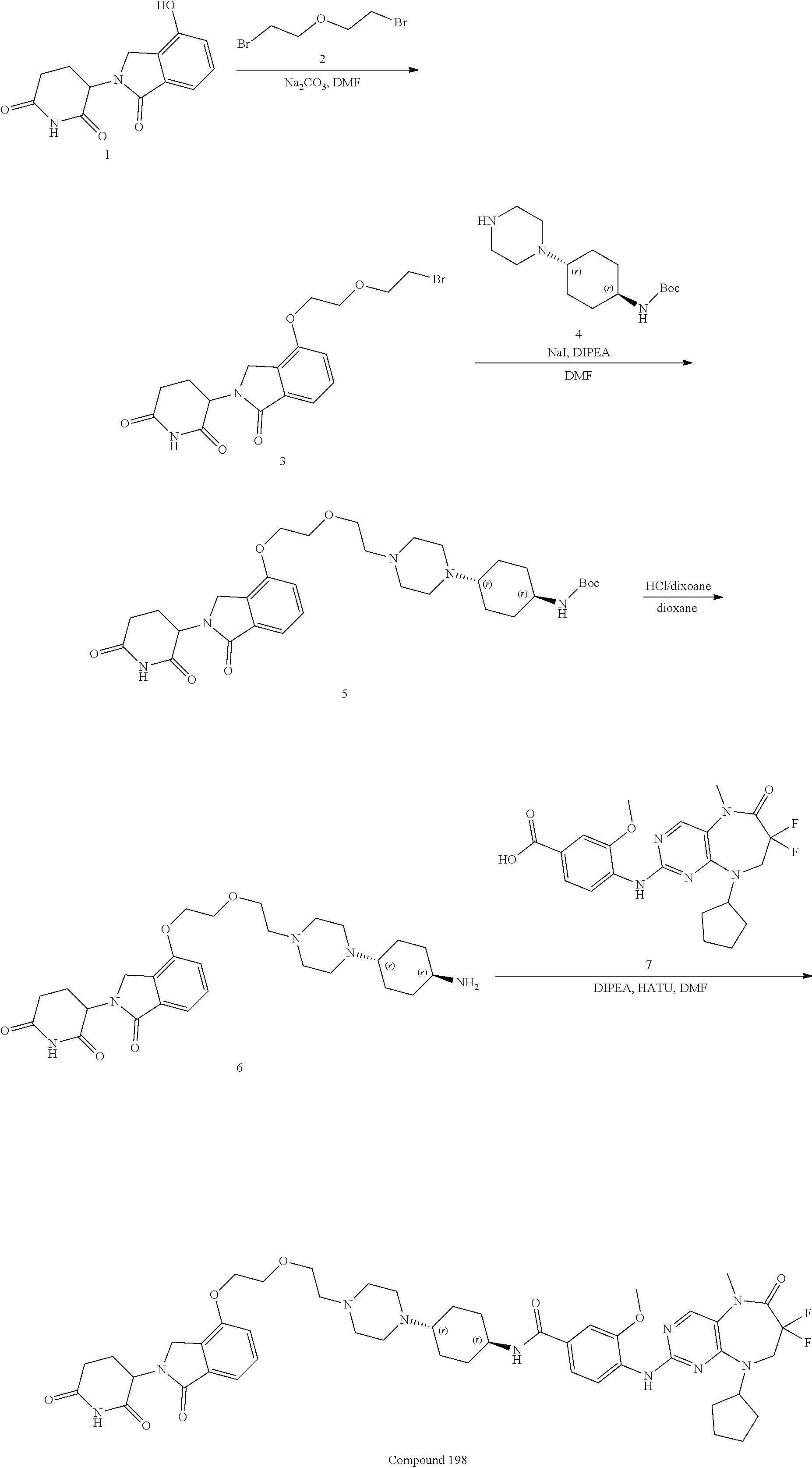

The compound 198 was synthesized by the method described in the scheme similar to the method described in Example 191.

MS(M+H)⁺=943.6, ¹H NMR (400 MHz, DMSO-d₆) δ=10.98 (br s, 1H), 8.29-8.24 (m, 2H), 8.06 (d, J=7.9 Hz, 1H), 7.95 (s, 1H), 7.52-7.45 (m, 3H), 7.32 (d, J=7.4 Hz, 1H), 7.27 (d, J=8.1 Hz, 1H), 5.16-5.08 (m, 1H), 4.76 (br t, J=7.8 Hz, 1H), 4.38-4.20 (m, 4H), 4.04 (br t, J=14.1 Hz, 2H), 3.93 (s, 3H), 3.78-3.66 (m, 4H), 3.60-3.56 (m, 2H), 3.31 (s, 3H), 3.02-2.88 (m, 2H), 2.63-2.55 (m, 4H), 2.44-2.38 (m, 6H), 2.21-2.13 (m, 1H), 2.00-1.86 (m, 5H), 1.82-1.78 (m, 2H), 1.71-1.68 (m, 2H), 1.64-1.58 (m, 4H), 1.42-1.24 (m, 4H).

### Example 199. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(6-((1-(2-(2,6-dioxopiperidin-3-yl)-1,3-dioxoisoindolin-4-yl)azetidin-3-yl)oxy)hexanoyl)piperidin-4-yl)-3-methoxybenzamide (Compound 199)

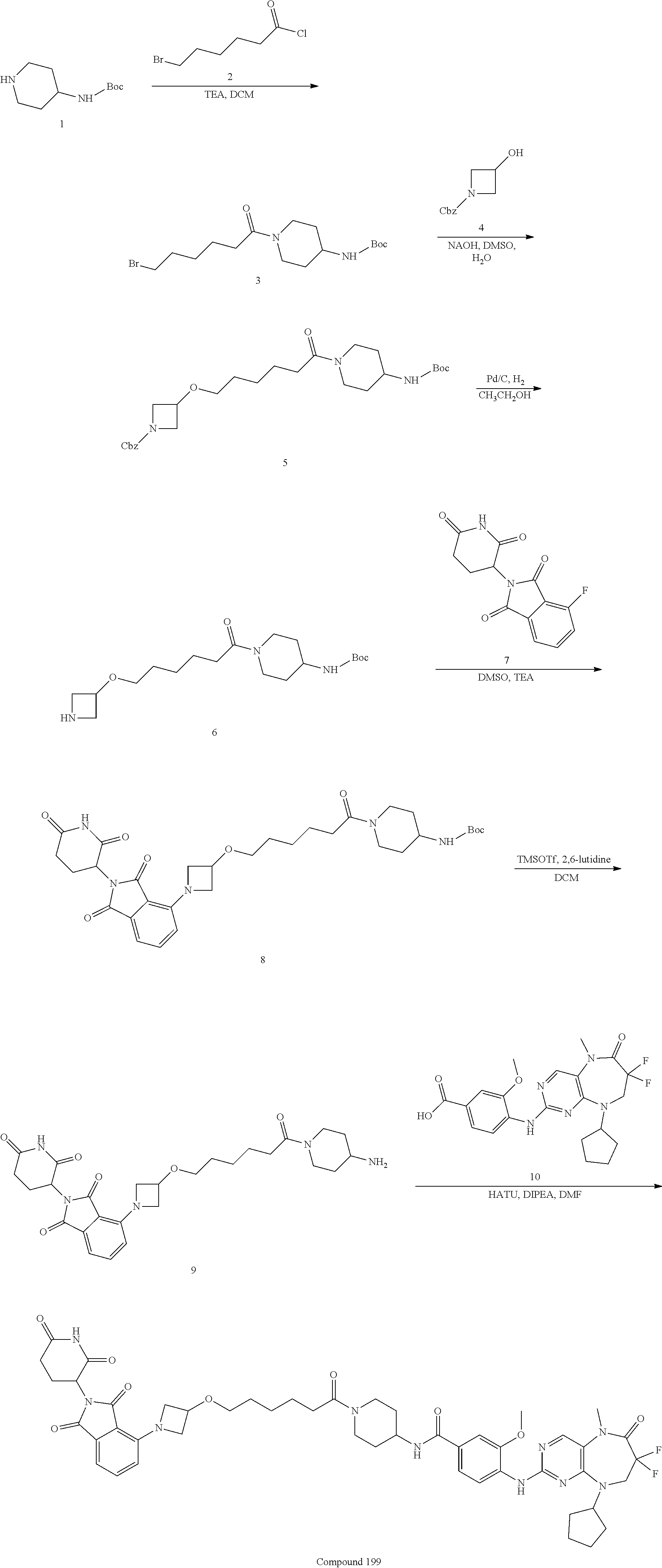

### Step 1. Synthesis of tert-butyl (1-(6-bromohexanoyl)piperidin-4-yl)carbamate (3)

To a solution of tert-butyl piperidin-4-ylcarbamate (2.06 g, 10.30 mmol) in DCM (10 mL) were added TEA (2.84 g, 28.10 mmol, 3.91 mL) and 6-bromohexanoyl chloride (2 g, 9.37 mmol) at 25° C. The mixture was stirred at 25° C. for 2 h. TLC (petroleum ether:EtOAc=1:2) indicated several new spots were formed. The reaction mixture was concentrated under reduced pressure to remove DCM. The residue was diluted with water (50 mL) and extracted with EtOAc (20 mL×3). The combined organic layers were washed with saturated brine (20 mL), dried over anhydrous sodium sulfate, filtered and concentrated under reduced pressure. The residue was purified by flash silica gel chromatography (ISCO®; 20 g Sepa Flash® Silica Flash Column, Eluent of 0˜50% petroleum ether:EtOAc gradient @80 mL/min) to afford tert-butyl (1-(6-bromohexanoyl)piperidin-4-yl)carbamate (2.6 g, 6.66 mmol, 71.13% yield, 96.7% purity) as yellow oil. MS(M−Boc+H)⁺=323.0

### Step 2. Synthesis of benzyl 3-((6-(4-((tert-butoxycarbonyl)amino)piperidin-1-yl)-6-oxohexyl)oxy)azetidine-1-carboxylate (5)

To a solution of tert-butyl (1-(6-bromohexanoyl)piperidin-4-yl)carbamate (1.0 g, 2.65 mmol) in DMSO (10 mL) were added a solution of NaOH (159.01 mg, 3.98 mmol) in water (4 mL) and benzyl 3-hydroxyazetidine-1-carboxylate (1.65 g, 7.95 mmol) at 25° C. The mixture was stirred at 25° C. for 12 h. TLC (petroleum ether:EtOAc=1:2) indicated several new spots were formed. The reaction mixture was diluted with water (50 mL) and extracted with EtOAc (20 mL×3). The combined organic layers were washed with saturated brine (20 mL), dried over anhydrous sodium sulfate, filtered and concentrated under reduced pressure. The residue was purified by flash silica gel chromatography (ISCO®; 20 g Sepa Flash® Silica Flash Column, Eluent of 0˜100% petroleum ether:EtOAc gradient @80 mL/min) to afford benzyl 3-((6-(4-((tert-butoxycarbonyl)amino)piperidin-1-yl)-6-oxohexyl)oxy)azetidine-1-carboxylate (1 g, 1.93 mmol, 72.90% yield, 97.3% purity) as yellow oil. MS(M+H)⁺=504.4

### Step 3. Synthesis of tert-butyl (1-(6-(azetidin-3-yloxy)hexanoyl)piperidin-4-yl)carbamate (6)

To a mixture of benzyl 3-((6-(4-((tert-butoxycarbonyl)amino)piperidin-1-yl)-6-oxohexyl)oxy)azetidine-1-carboxylate (1 g, 1.99 mmol) in CF₃CH₂OH (10 mL) were added Pd/C (0.2 g, 397.12 μmol, 10% purity) at 25° C. under N₂ atmosphere. Then the mixture was stirred at 25° C. for 12 h under H₂ (15 Psi). TLC (petroleum ether:EtOAc=1:2) indicated one new spot was formed. The mixture was filtered to remove the catalyst. The filtrate was concentrated under reduced pressure to afford tert-butyl (1-(6-(azetidin-3-yloxy)hexanoyl)piperidin-4-yl)carbamate (0.8 g, 1.89 mmol, 95.19% yield, 87.3% purity) as light yellow oil. MS(M+H)⁺=370.2

### Step 4. Synthesis of tert-butyl (1-(6-((1-(2-(2,6-dioxopiperidin-3-yl)-1,3-dioxoisoindolin-4-yl)azetidin-3-yl)oxy)hexanoyl)piperidin-4-yl)carbamate (8)

To a solution of tert-butyl (1-(6-(azetidin-3-yloxy)hexanoyl)piperidin-4-yl)carbamate (0.8 g, 2.17 mmol) in DMSO (10 mL) were added TEA (657.25 mg, 6.50 mmol, 904.06 μL) and 2-(2,6-dioxopiperidin-3-yl)-4-fluoroisoindoline-1,3-dione (598.04 mg, 2.17 mmol) at 25° C. The mixture was stirred at 90° C. for 6 h. LCMS showed the reaction was completed. The reaction mixture was diluted with water (50 mL) and extracted with EtOAc (20 mL×3). The combined organic layers were washed with saturated brine (20 mL), dried over anhydrous sodium sulfate, filtered and concentrated under reduced pressure. The residue was purified by flash silica gel chromatography (ISCO®; 20 g Sepa Flash® Silica Flash Column, eluent of 0˜100% petroleum ether:EtOAc gradient @60 mL/min) to afford tert-butyl (1-(6-((1-(2-(2,6-dioxopiperidin-3-yl)-1,3-dioxoisoindolin-4-yl)azetidin-3-yl)oxy)hexanoyl)piperidin-4-yl)carbamate (1.1 g, 1.69 mmol, 77.87% yield, 95.9% purity) as yellow oil. MS(M+H)⁺=626.2

### Step 5. Synthesis of 4-(3-((6-(4-aminopiperidin-1-yl)-6-oxohexyl)oxy)azetidin-1-yl)-2-(2,6-dioxopiperidin-3-yl)isoindoline-1,3-dione (9)

To a solution of tert-butyl (1-(6-((1-(2-(2,6-dioxopiperidin-3-yl)-1,3-dioxoisoindolin-4-yl)azetidin-3-yl)oxy)hexanoyl)piperidin-4-yl)carbamate (1.1 g, 1.76 mmol) in DCM (10 mL) were added TMSOTf (976.82 mg, 4.40 mmol, 794.17 μL) and 2,6-lutidine (753.50 mg, 7.03 mmol, 819.02 μL) at 0° C. The mixture was stirred at 25° C. for 2 h. TLC (petroleum ether:EtOAc=10:1) indicated one new spot was formed. The mixture was concentrated under reduced pressure to afford 4-(3-((6-(4-aminopiperidin-1-yl)-6-oxohexyl)oxy)azetidin-1-yl)-2-(2,6-dioxopiperidin-3-yl)isoindoline-1,3-dione (3 g, crude) as yellow oil. MS(M+H)⁺=526.1

### Step 6. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(6-((1-(2-(2,6-dioxopiperidin-3-yl)-1,3-dioxoisoindolin-4-yl)azetidin-3-yl)oxy)hexanoyl)piperidin-4-yl)-3-methoxybenzamide (Compound 199)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (200 mg, 446.99 μmol) in DMF (5 mL) were added DIPEA (173.31 mg, 1.34 mmol, 233.57 μL) and HATU (339.92 mg, 893.99 μmol) at 25° C. Then 4-(3-((6-(4-aminopiperidin-1-yl)-6-oxohexyl)oxy)azetidin-1-yl)-2-(2,6-dioxopiperidin-3-yl)isoindoline-1,3-dione (469.87 mg, 893.99 μmol) was added to the mixture after 0.5 h. The mixture was stirred at 25° C. for 12 h. LCMS showed a main peak with desired mass. The reaction mixture was diluted with water (50 mL) and extracted with EtOAc (20 mL×3). The combined organic layers were washed with saturated brine (30 mL), dried over anhydrous sodium sulfate, filtered and concentrated under reduced pressure. The residue was purified by flash silica gel chromatography (ISCO®; 15 g SepaFlash® Silica Flash Column, Eluent of 0˜60% petroleum ether: (EtOAc/methanol=2/1) gradient @50 mL/min) followed by prep-HPLC (column: Waters Xbridge 150×25 mm×5 μm; mobile phase: [water(NH₄HCO₃)-ACN]; B %: 40%-70%, 9 min) and prep-HPLC (column: Waters Xbridge 150×25 mm×5 μm; mobile phase: [water(NH₄HCO₃)-ACN]; B %: 43%-73%, 10 min). The eluents were lyophilized to afford 4-((9-cyclopentyl-7,7-difluoro-5-methyl-6-oxo-6,7,8,9-tetrahydro-5H-pyrimido[4,5-b][1,4]diazepin-2yl)amino)-N-(1-(6-((1-(2-(2,6-dioxopiperidin-3-yl)-1,3-dioxoisoindolin-4-yl)azetidin-3-yl)oxy) hexanoyl)piperidin-4-yl)-3-methoxybenzamide (80 mg, 81.67 μmol, 66.10% yield, 97.5% purity) as a yellow solid. MS(M+H)⁺=955.4.

¹H NMR (400 MHz, DMSO-d₆) δ=11.05 (brs, 1H), 8.22 (s, 1H), 8.14 (d, J=8.0 Hz, 1H), 7.98 (s, 1H), 7.77 (d, J=8.0 Hz, 1H), 7.57-7.53 (m, 1H), 7.11 (d, J=6.8 Hz, 1H), 7.01 (d, J=0.8 Hz, 1H), 6.94-6.92 (m, 1H), 6.78 (d, J=8.4 Hz, 1H), 5.06-5.02 (m, 1H), 4.72-4.68 (m, 1H), 4.40-4.33 (m, 3H), 4.05-3.94 (m, 5H), 3.87-3.80 (m, 5H), 3.41-3.40 (m, 1H), 3.31 (s, 3H), 3.08-3.01 (m, 1H), 2.90-2.81 (m, 1H), 2.59-2.53 (m, 2H), 2.49-2.44 (m, 2H), 2.05 (t, J=7.2 Hz, 2H), 2.02-1.96 (m, 1H), 1.93-1.85 (m, 2H), 1.78-1.72 (m, 2H), 1.69-1.64 (m, 2H), 1.60-1.46 (m, 8H), 1.36-1.25 (m, 4H).

### Example 200. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(1-(2-(2,6-dioxopiperidin-3-yl)-1,3-dioxoisoindolin-4-yl)azetidin-3-yl)piperazin-1-yl)-3-methoxybenzamide (Compound 200)

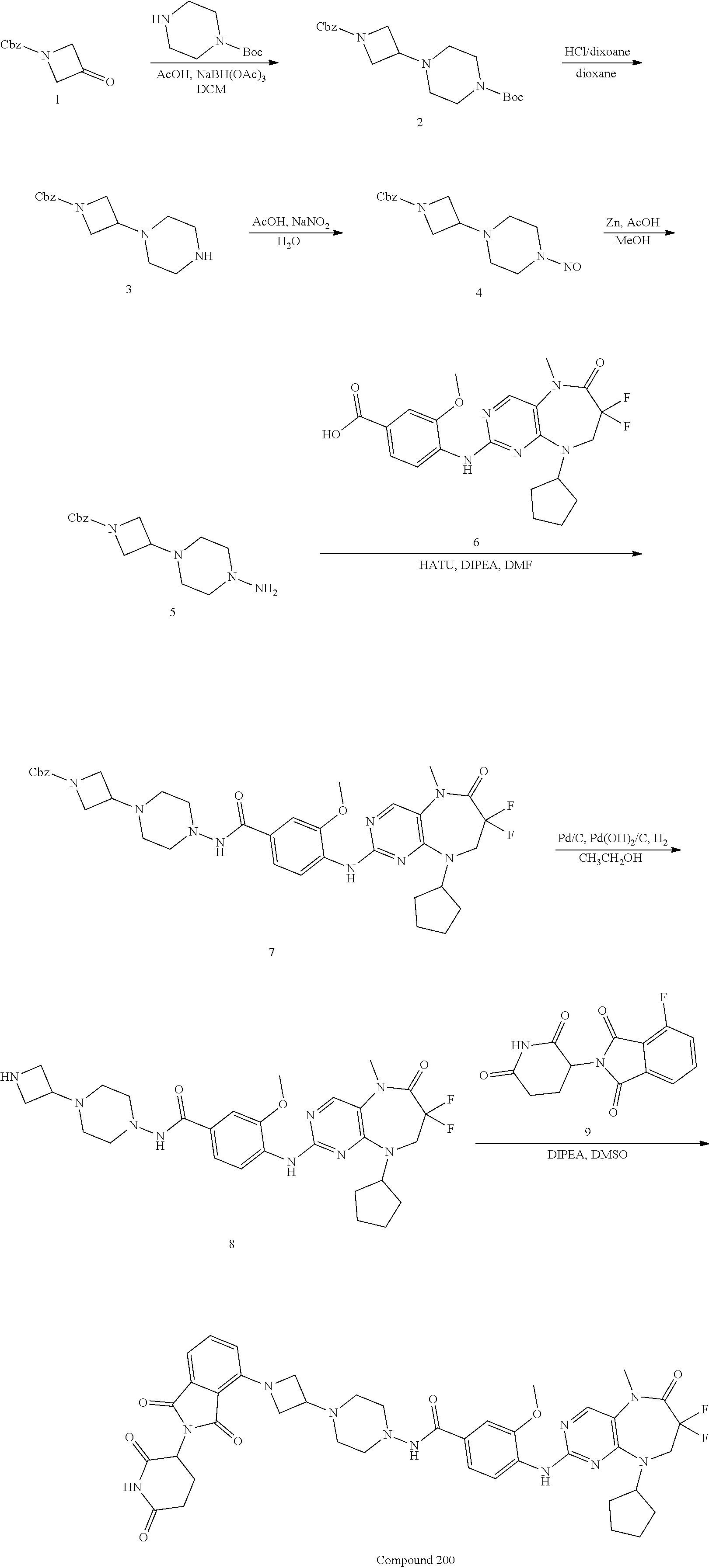

### Step 1. Synthesis of tert-butyl 4-(1-((benzyloxy)carbonyl)azetidin-3-yl)piperazine-1-carboxylate (2)

To a solution of benzyl 3-oxoazetidine-1-carboxylate (10 g, 48.73 mmol) and tert-butyl piperazine-1-carboxylate (9.08 g, 48.73 mmol) in DCM (100 mL) was added AcOH (2.93 g, 48.73 mmol, 2.79 mL) at 20° C., then was slowly added NaBH(OAc)₃ (30.98 g, 146.19 mmol) and the resulting mixture was stirred at 20° C. for 12 h. LCMS showed starting material was consumed completely and 51% peak with desired mass was detected. The reaction mixture was diluted with H₂O (200 mL) and extracted with DCM (100 mL×3). The organic layer was dried over Na₂SO₄, filtered and concentrated. The residue was purified by flash silica gel chromatography (80 g SepaFlash® Silica Flash Column, Eluent of 0˜33% EtOAc/Petroleum ether gradient @100 mL/min) to afford tert-butyl 4-(1-((benzyloxy)carbonyl)azetidin-3-yl)piperazine-1-carboxylate (13.9 g, 37.02 mmol, 75.97% yield) as a yellow oil. MS(M+H)⁺=376.2

### Step 2. Synthesis of benzyl 3-(piperazin-1-yl)azetidine-1-carboxylate (3)

To a solution of tert-butyl 4-(1-((benzyloxy)carbonyl)azetidin-3-yl)piperazine-1-carboxylate (5 g, 13.32 mmol) in dioxane (20 mL) was added HCl/dioxane (4 M, 50.00 mL) at 20° C. and the resulting mixture was stirred at 20° C. for 1 h. LCMS showed starting material was consumed completely and 71% peak with desired mass was detected. The reaction mixture was concentrated in vacuum to afford benzyl 3-(piperazin-1-yl)azetidine-1-carboxylate (4.2 g, crude, HCl) as a white solid. MS(M+H)⁺=276.2

### Step 3. Synthesis of benzyl 3-(4-nitrosopiperazin-1-yl)azetidine-1-carboxylate (4)

To a solution of benzyl 3-piperazin-1-yl)azetidine-1-carboxylate (4.2 g, crude, HCl) in H₂O (60 mL) was added NaNO₂ (1.86 g, 26.94 mmol) at 0° C., then AcOH (2.43 g, 40.41 mmol, 2.31 mL) was added drop-wise at 0° C. and the resulting mixture was stirred at 20° C. for 12 h. LCMS showed starting material was consumed completely and 91% peak with desired mass was detected. Saturated NaHCO₃ (60 mL) was added to this reaction mixture to adjust the pH=9 and extracted with EtOAc (60 mL×3). The organic layer was dried over Na₂SO₄, filtered and concentrated to afford benzyl 3-(4-nitrosopiperazin-1-yl)azetidine-1-carboxylate (3.9 g, 12.30 mmol, 91.33% yield, 96% purity) as a yellow oil. MS(M+H)⁺=305.2

### Step 4. Synthesis of benzyl 3-(4-aminopiperazin-1-yl)azetidine-1-carboxylate (5)

To a solution of benzyl 3-(4-nitrosopiperazin-1-yl)azetidine-1-carboxylate (3.9 g, 12.81 mmol) in MeOH (100 mL) was added Zn (4.36 g, 66.64 mmol) slowly at 0° C., then AcOH (11.54 g, 192.22 mmol, 10.99 mL) was added drop-wise at 0° C. and the resulting mixture was stirred at 20° C. for 4 h. LCMS showed starting material was consumed completely and 80% peak with desired mass was detected. The reaction mixture was diluted with MeOH (100 mL) and filtered. The filtrate was concentrated in vacuum to afford benzyl 3-(4-aminopiperazin-1-yl)azetidine-1-carboxylate (3.72 g, crude) as a yellow solid. MS(M+H)⁺=291.2

### Step 5. Synthesis of N-(4-(azetidin-3-yl)piperazin-1-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (7)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (500 mg, 1.12 mmol) in DMF (5 mL) were added HATU (467.39 mg, 1.23 mmol) and DIPEA (288.85 mg, 2.23 mmol, 389.29 μL), the mixture was stirred at 20° C. for 10 min and a solution of benzyl 3-(4-aminopiperazin-1-yl)azetidine-1-carboxylate (454.26 mg, 1.56 mmol) and DIPEA (433.28 mg, 3.35 mmol, 583.94 μL, 3 eq) in DMF (5 mL) was added and the resulting mixture was stirred at 20° C. for 1 h. LCMS showed starting material was consumed completely and 63% peak with desired mass was detected. The reaction mixture was diluted with H₂O (30 mL) and extracted with EtOAc (30 mL×3). The organic layer was dried over Na₂SO₄, filtered and concentrated. The residue was purified by flash silica gel chromatography (25 g SepaFlash® Silica Flash Column, Eluent of 0˜100% EtOAc/Petroleum ether 0˜5% Dichloromethane/Methanol gradient @100 mL/min) to afford N-(4-(azetidin-3-yl)piperazin-1-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (783 mg, 1.09 mmol, 97.35% yield) as a yellow oil. MS(M+H)⁺=720.2

¹H NMR (400 MHz, DMSO-d₆) δ=9.64-9.22 (m, 1H), 8.33-8.22 (m, 2H), 7.98 (s, 1H), 7.50-7.26 (m, 7H), 5.05 (s, 2H), 4.76 (q, J=7.8 Hz, 1H), 4.29-3.62 (m, 10H), 3.33 (s, 3H), 3.12-2.84 (m, 4H), 2.52 (s, 2H), 2.49-2.34 (m, 2H), 2.00-1.88 (m, 2H), 1.77-1.62 (m, 2H), 1.66-1.52 (m, 4H)

### Step 6. Synthesis of N-(4-(azetidin-3-yl)piperazin-1-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (8)

To a solution of N-(4-(azetidin-3-yl)piperazin-1-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (703 mg, 976.69 μmol) in CF₃CH₂OH (15 mL) was added Pd/C (200 mg, 10% purity) under N₂ atmosphere. The suspension was degassed and purged with H₂ for 3 times and the resulting mixture was stirred under H₂ (15 Psi) at 20° C. for 16 h. LCMS showed no reaction and Pd(OH)₂/C (200 mg, 20% purity) was added to this reaction mixture under N₂ atmosphere. The suspension was degassed and purged with H₂ for 3 times and the resulting mixture was stirred under H₂ (15 Psi) at 40° C. for 16 h. LCMS showed 58% of starting material remained and 30% peak with desired mass was detected and Pd(OH)₂/C (100 mg, 20% purity) was added to this reaction mixture under N₂ atmosphere. The suspension was degassed and purged with H₂ for 3 times. The mixture was stirred under H₂ (15 Psi) at 50° C. for 16 h. LCMS showed 17% of starting material remained and 81% peak with desired mass was detected. The reaction mixture was diluted with CF₃CH₂OH (40 mL) and filtered. The filtrate was concentrated in vacuum to afford N-(4-(azetidin-3-yl)piperazin-1-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (321 mg, 548.11 μmol, 56.12% yield) as a yellow oil. MS(M+H)⁺=586.3

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(1-(2-(2,6-dioxopiperidin-3-yl)-1,3-dioxoisoindolin-4-yl)azetidin-3-yl)piperazin-1-yl)-3-methoxybenzamide (Compound 200)

To a solution of 2-(2,6-dioxo-3-piperidyl)-4-fluoro-isoindoline-1,3-dione (50 mg, 181.02 μmol) in DMSO (3 mL) were added DIPEA (70.18 mg, 543.05 μmol, 94.59 μL) and N-(4-(azetidin-3-yl)piperazin-1-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (116.61 mg, 199.12 μmol) at 20° C. and the resulting mixture was stirred at 80° C. for 16 h. LCMS showed 54% of 2-(2,6-dioxo-3-piperidyl)-4-fluoro-isoindoline-1,3-dione remained and 37% peak with desired mass was detected. The reaction mixture was diluted with H₂O (12 mL) and extracted with EtAOc (12 mL×3). The organic layer was washed with brine (12 mL×3), dried over Na₂SO₄, filtered and concentrated. The residue was purified by prep-HPLC (column: Unisil 3-100 C18 Ultra 150*50 mm*3 μm; mobile phase: [water(FA)-ACN]; B %: 16%-46%, 7 min) and re-purified by prep-HPLC (column: Waters Xbridge 150*25 mm*5 μm; mobile phase: [water(NH₄HCO)-ACN]; B %: 36%-66%, 8 min) and lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(1-(2-(2,6-dioxopiperidin-3-yl)-1,3-dioxoisoindolin-4-yl)azetidin-3-yl)piperazin-1-yl)-3-methoxybenzamide (21.1 mg, 24.81 μmol, 13.71% yield, 99% purity) as a yellow solid. MS(M+H)⁺=842.1

¹H NMR (400 MHz, DMSO-d₆) δ=11.07 (s, 1H), 9.39 (s, 1H), 8.30-8.23 (m, 2H), 7.97 (s, 1H), 7.62-7.55 (m, 1H), 7.45-7.38 (m, 2H), 7.13 (d, J=7.0 Hz, 1H), 6.81 (d, J=8.5 Hz, 1H), 5.05 (dd, J=5.5, 12.6 Hz, 1H), 4.83-4.70 (m, 1H), 4.38-4.20 (m, 2H), 4.09-3.95 (m, 4H), 3.93 (s, 3H), 3.32-3.22 (m, 4H), 3.01-2.82 (m, 5H), 2.63-2.52 (m, 6H), 2.05-1.89 (m, 3H), 1.74-1.68 (m, 2H), 1.65-1.52 (m, 4H)

### Example 201. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(1-(2-(2,6-dioxopiperidin-3-yl)-1,3-dioxoisoindolin-5-yl)azetidin-3-yl)piperazin-1-yl)-3-methoxybenzamide (Compound 201)

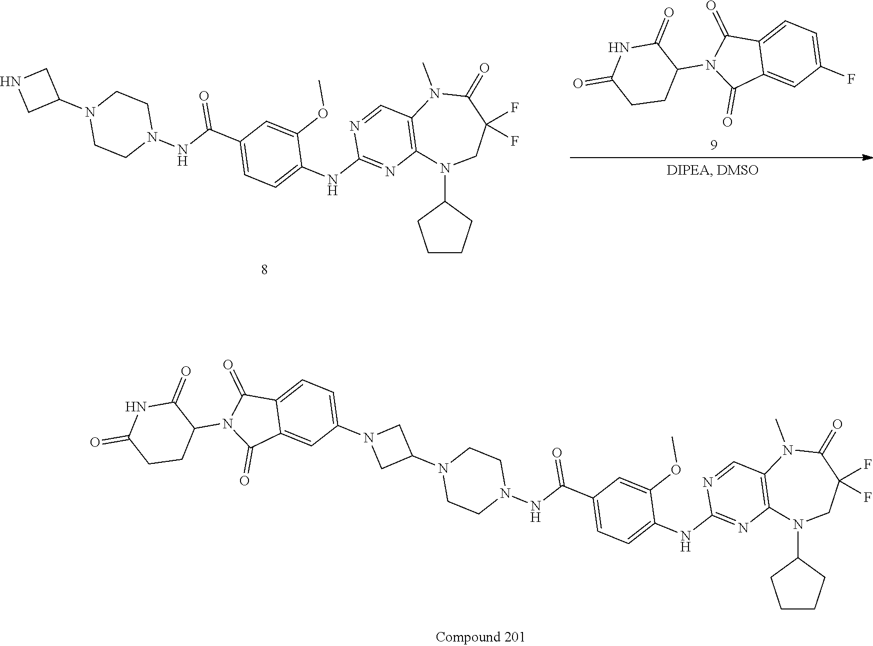 [SMILES: COc1cc(C(=O)NN2CCN(C3CNC3)CC2)ccc1Nc1ncc2c(n1)N(C1CCCC1)CC(F)(F)C(=O)N2C ; O=C1CCC(N2C(=O)c3ccc(F)cc3C2=O)C(=O)N1 ; COc1cc(C(=O)NN2CCN(C3CN(c4ccc5c(c4)C(=O)N(C4CCC(=O)NC4=O)C5=O)C3)CC2)ccc1Nc1ncc2c(n1)N(C1CCCC1)CC(F)(F)C(=O)N2C]

To a solution of 2-(2,6-dioxo-3-piperidyl)-5-fluoro-isoindoline-1,3-dione (70 mg, 253.42 μmol) and N-(4-(azetidin-3-yl)piperazin-1-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (155.84 mg, 266.09 μmol) in DMSO (5 mL) were added DIPEA (98.26 mg, 760.26 μmol, 132.42 μL) at 20° C. and the resulting mixture was stirred at 80° C. for 12 h. LCMS showed 61% of 2-(2,6-dioxo-3-piperidyl)-5-fluoro-isoindoline-1,3-dione remained and 33% peak with desired mass was detected. The reaction mixture was diluted with H₂O (12 mL) and extracted with EtAOc (12 mL×3). The organic layer was washed with brine (12 mL×3), dried over Na₂SO₄, filtered and concentrated. The residue was purified by prep-HPLC (column: Unisil 3-100 C18 Ultra 150*50 mm*3 μm; mobile phase: [water(FA)-ACN]; B %: 16%-46%, 7 min) and re-purified by prep-HPLC (column: Waters Xbridge 150*25 mm*5 μm; mobile phase: [water(NH₄HCO₃)-ACN]; B %: 36%-66%, 8 min) and lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(1-(2-(2,6-dioxopiperidin-3-yl)-1,3-dioxoisoindolin-5-yl)azetidin-3-yl)piperazin-1-yl)-3-methoxybenzamide (25.3 mg, 29.75 μmol, 11.74% yield, 99% purity) as a yellow solid. MS(M+H)⁺=842.1

¹H NMR (400 MHz, CD₃CN) δ=8.94 (br s, 1H), 8.47 (d, J=8.4 Hz, 1H), 8.10 (s, 1H), 7.86 (s, 1H), 7.71 (s, 1H), 7.64-7.57 (m, 1H), 7.42-7.33 (m, 2H), 6.78 (d, J=1.9 Hz, 1H), 6.62 (dd, J=2.0, 8.4 Hz, 1H), 4.93 (dd, J=5.1, 12.3 Hz, 1H), 4.89-4.81 (m, 1H), 4.15-4.07 (m, 2H), 4.01-3.91 (m, 5H), 3.86 (dd, J=5.0, 8.4 Hz, 2H), 3.39 (q, J=5.7 Hz, 1H), 3.33 (s, 3H), 3.02-2.96 (m, 4H), 2.82-2.44 (m, 7H), 2.12-2.06 (m, 1H), 2.06-1.98 (m, 2H), 1.81-1.71 (m, 2H), 1.71-1.56 (m, 4H).

### Example 202. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-((2-(2,6-dioxopiperidin-3-yl)-1,3-dioxoisoindolin-4-yl)methyl)azetidin-3-yl)ethyl)piperidin-4-yl)-3-methoxybenzamide (Compound 202)

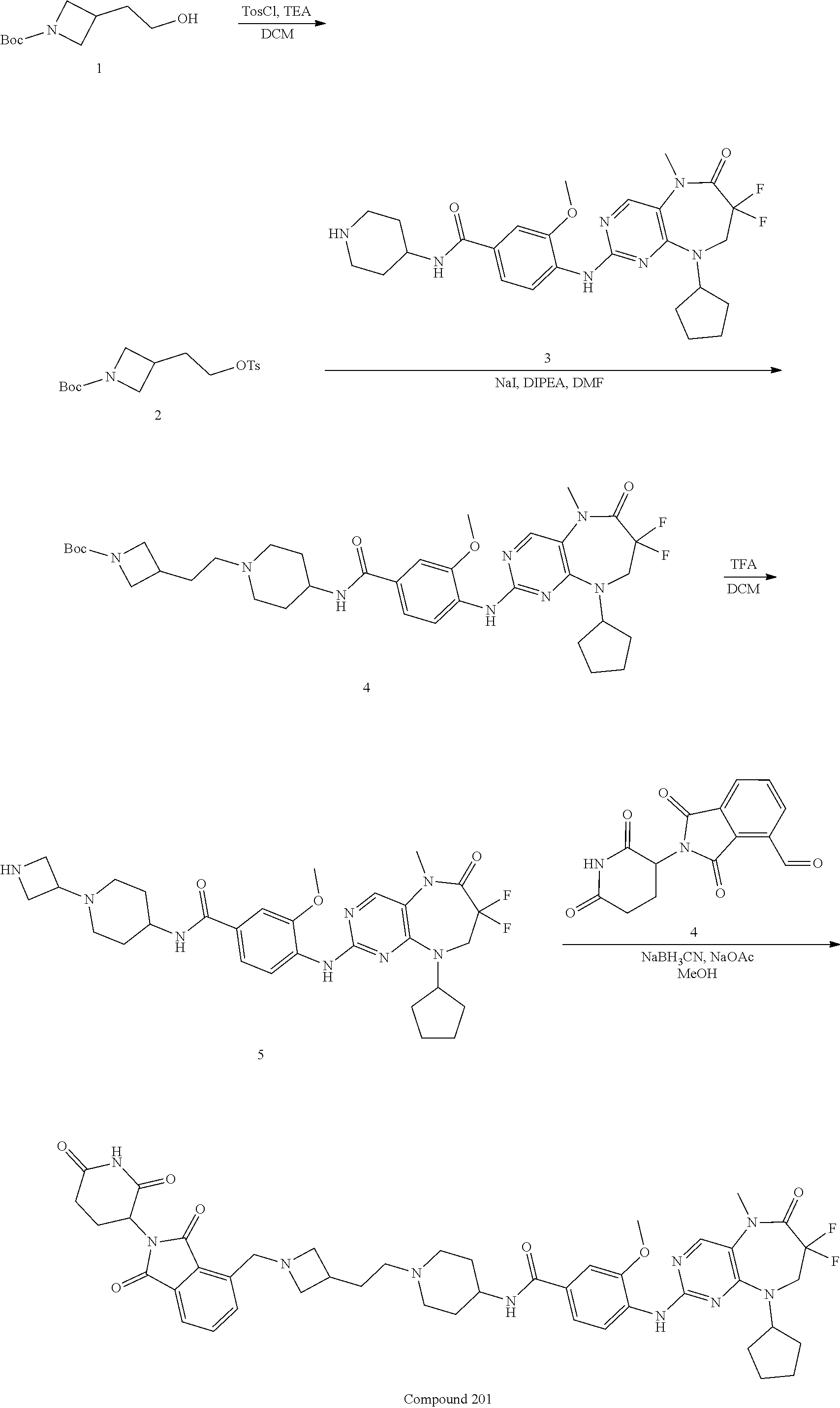

The compound 202 was synthesized by the method described in the scheme similar to the method described in Example 200.

MS(M+H)⁺=883.1, ¹H NMR (400 MHz, DMSO-d₆) δ=11.14 (br s, 1H), 8.34-8.28 (m, 2H), 8.27 (s, 1H), 8.00 (s, 1H), 7.93 (br d, J=6.6 Hz, 1H), 7.89-7.79 (m, 2H), 7.49 (br s, 2H), 5.14 (td, J=2.6, 12.7 Hz, 1H), 4.83-4.72 (m, 1H), 4.22-3.99 (m, 5H), 3.94 (s, 3H), 3.90-3.83 (m, 1H), 3.78-3.66 (m, 2H), 3.56 (br s, 8H), 3.33 (s, 3H), 2.97-2.81 (m, 1H), 2.64-2.57 (m, 2H), 2.29-2.17 (m, 1H), 2.10-2.00 (m, 3H), 2.00-1.87 (m, 4H), 1.87-1.78 (m, 1H), 1.76-1.66 (m, 2H), 1.66-1.53 (m, 4H).

### Example 203. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(1-((2-(2,6-dioxopiperidin-3-yl)-1,3-dioxoisoindolin-4-yl)methyl)azetidin-3-yl)propanoyl)piperidin-4-yl)-3-methoxybenzamide (Compound 203)

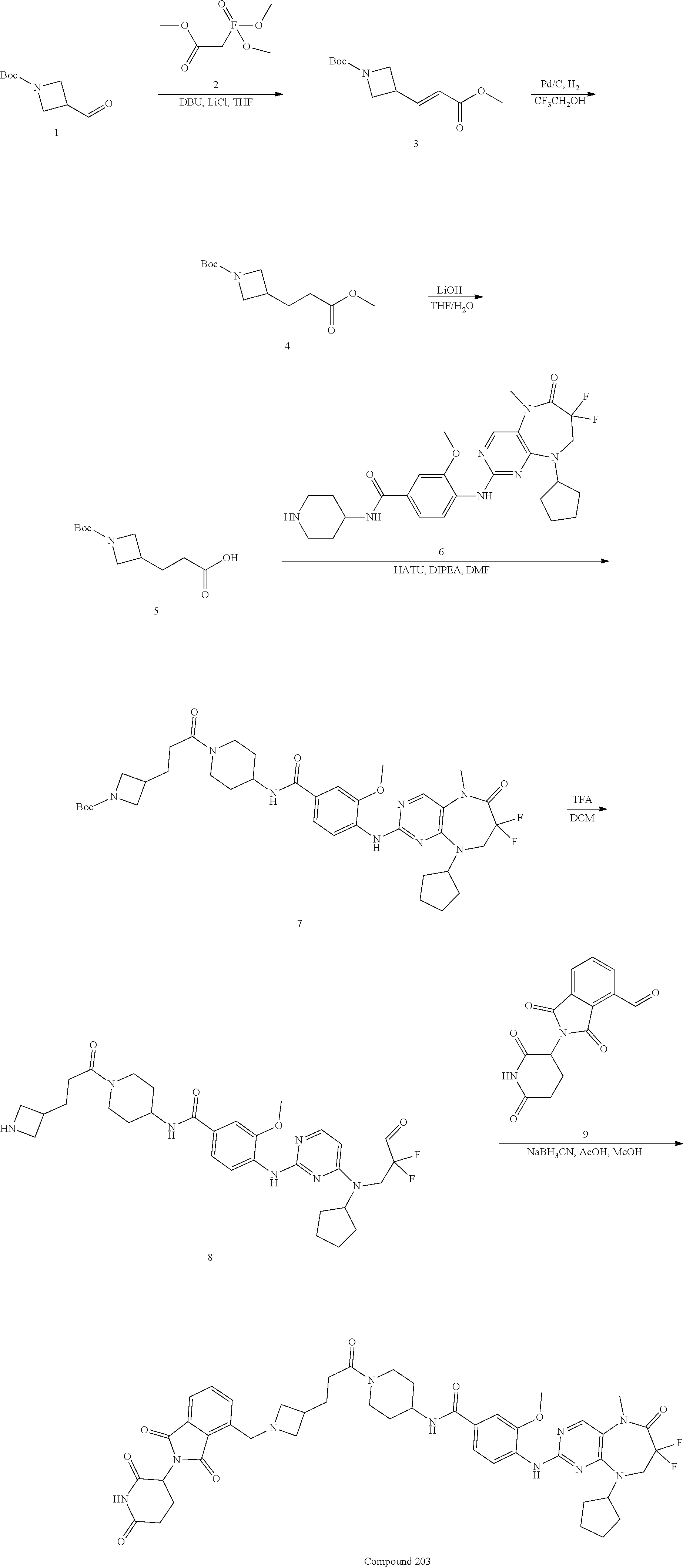

### Step 1. Synthesis of tert-butyl (E)-3-(3-methoxy-3-oxoprop-1-en-1-yl)azetidine-1-carboxylate (3)

To a solution of methyl 2-(dimethoxyphosphoryl)acetate (1.18 g, 6.48 mmol, 936.90 L) in THF (10 mL) were added DBU (904.12 mg, 5.94 mmol, 895.17 μL) and LiCl (251.22 mg, 5.93 mmol, 121.36 μL) at 0° C., the mixture was stirred at 0° C. for 10 min. A solution of tert-butyl 3-formylazetidine-1-carboxylate (1 g, 5.40 mmol) in THF (10 mL) was added at 0° C., the resulting mixture was stirred at 25° C. for 30 min. LCMS showed a main peak with desired mass. The reaction mixture was diluted with NH₄Cl (20 mL), extracted with EtOAc (20 mL×3). The combined organic layers were washed with brine (20 mL×2), dried over Na₂SO₄, filtered. The filtrate was concentrated under reduced pressure to give a residue. The residue was purified by flash silica gel chromatography (Biotage; 20 g SepaFlash® Silica Flash Column, Eluent of 0˜15% EtOAcEtOAc/Petroleum ether gradient @40 mL/min) to afford tert-butyl (E)-3-(3-methoxy-3-oxoprop-1-en-1-yl)azetidine-1-carboxylate (1.2 g, 4.97 mmol, 92.12% yield, 100% purity) as colorless oil. MS(M−56+H)⁺=186.0

### Step 2. Synthesis of tert-butyl 3-(3-methoxy-3-oxopropyl)azetidine-1-carboxylate (4)

A mixture of tert-butyl (E)-3-(3-methoxy-3-oxoprop-1-en-1-yl)azetidine-1-carboxylate (1.2 g, 4.97 mmol) and Pd/C (200 mg, 10% purity) in CF₃CH₂OH (10 mL) was degassed and purged with H₂ for 3 times, the resulting mixture was stirred at 20° C. for 16 h under H₂ (15 Psi) atmosphere. LCMS showed a main peak with desired mass. The mixture was filtered through a pad of celite. The filtrate was concentrated in vacuum to afford tert-butyl 3-(3-methoxy-3-oxopropyl)azetidine-1-carboxylate (1.2 g, crude) as colorless oil. MS(M−56+H)⁺=188.0

### Step 3. Synthesis of 3-(1-(tert-butoxycarbonyl)azetidin-3-yl)propanoic acid (5)

To a solution of tert-butyl 3-(3-methoxy-3-oxopropyl)azetidine-1-carboxylate (500 mg, 2.06 mmol) in THF (10 mL) and H₂O (2 mL) was added LiOH·H₂O (172.48 mg, 4.11 mmol), the mixture was stirred at 25° C. for 2 h. LCMS showed a main peak with desired mass. The mixture was concentrated under reduced pressure to afford 3-(1-(tert-butoxycarbonyl)azetidin-3-yl)propanoic acid (480 mg, crude, Li) as a white solid. MS(M−56+H)⁺=174.5

### Step 4. Synthesis of tert-butyl 3-(3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperidin-1-yl)-3-oxopropyl)azetidine-1-carboxylate (7)

To a solution of 3-(1-(tert-butoxycarbonyl)azetidin-3-yl)propanoic acid (143.39 mg, 625.43 μmol) in DMF (5 mL) were added DIPEA (242.50 mg, 1.88 mmol, 326.81 μL), HATU (356.71 mg, 938.14 μmol), the mixture was stirred at 25° C. for 0.5 h, 4-((9-cyclopentyl-7,7-difluoro-5-methyl-6-oxo-6,7,8,9-tetrahydro-5H-pyrimido[4,5-b][1,4]diazepin-2-yl)amino)-3-methoxy-N-(piperidin-4-yl)benzamide (360 mg, 625.43 μmol, 1 eq, FA) was added, the resulting mixture was stirred at 25° C. for 1 h. LCMS showed a main peak with desired mass. The reaction mixture was diluted with H₂O (20 mL), extracted with EtOAc (20 mL×3). The combined organic layers were washed with brine (20 mL×2), dried over Na₂SO₄, filtered. The filtrate was concentrated under reduced pressure to give a residue. The residue was purified by flash silica gel chromatography (ISCO®; 40 g SepaFlash® Silica Flash Column, Eluent of 0˜20% Ethylacetate/MeOH @40 mL/min) to afford tert-butyl 3-(3-(4-(4-((9-cyclopentyl-7,7-difluoro-5-methyl-6-oxo-6,7,8,9-tetrahydro-5H-pyrimido [4,5-b][1,4]diazepin-2-yl)amino)-3-methoxybenzamido)piperidin-1-yl)-3-oxopropyl)azetidine-1-carboxylate (430 mg, 568.82 μmol, 90.95% yield, 98% purity) as a yellow solid. MS(M+H)⁺=741.5

### Step 5. Synthesis of N-(1-(3-(azetidin-3-yl)propanoyl)piperidin-4-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (8)

To a solution of tert-butyl 3-(3-(4-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperidin-1-yl)-3-oxopropyl)azetidine-1-carboxylate (300 mg, 404.95 μmol) in DCM (10 mL) was added TFA (1.54 g, 13.51 mmol, 1.0 mL), the mixture was stirred at 0° C. for 0.5 h. LCMS showed a peak (32%) with desired mass. The mixture was concentrated in vacuum. The residue was purified by reversed-phase HPLC (column: Phenomenex Synergi C18 150*25 mm*10 μm; mobile phase: [water(FA)-ACN]; B %: 14%-34%, 10 min). The eluent was lyophilized to afford N-[1-[3-(azetidin-3-yl)propanoyl]-4-piperidyl]-4-[(9-cyclopentyl-7,7-difluoro-5-methyl-6-oxo-8H-pyrimido[4,5-b][1,4]diazepin-2-yl)amino]-3-methoxy-benzamide (100 mg, 131.05 μmol, 32.36% yield, 90% purity, FA) as a white solid. MS(M+H)⁺=641.4

### Step 6. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(1-((2-(2,6-dioxopiperidin-3-yl)-1,3-dioxoisoindolin-4-yl)methyl)azetidin-3-yl)propanoyl)piperidin-4-yl)-3-methoxybenzamide (Compound 203)

To a solution of N-(1-(3-(azetidin-3-yl)propanoyl)piperidin-4-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (80 mg, 116.49 μmol, FA) and 2-(2,6-dioxopiperidin-3-yl)-1,3-dioxoisoindoline-4-carbaldehyde (166.72 mg, 582.46 μmol) in MeOH (0.5 mL) was added AcOH (13.99 mg, 232.98 μmol, 13.32 μL), the mixture was stirred at 20° C. for 2 h, NaBH₃CN (21.96 mg, 349.47 μmol) was added, the mixture was stirred at 20° C. for 16 h. LCMS showed a peak (22%) with desired mass. The reaction mixture was concentrated under reduced pressure to give a residue. The residue was purified by prep-HPLC (column: Phenomenex Synergi C18 150*25 mm*10 μm; mobile phase: [water(FA)-ACN]; B %: 19%-39%, 10 min) to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(3-(1-((2-(2,6-dioxopiperidin-3-yl)-1,3-dioxoisoindolin-4-yl)methyl)azetidin-3-yl)propanoyl)piperidin-4-yl)-3-methoxybenzamide (10.9 mg, 10.17 μmol, 8.73% yield, 85% purity) as a yellow solid. MS(M+H)⁺=911.5.

¹H NMR (400 MHz, CD₃CN) δ=9.04 (s, 1H), 8.47 (d, J=8.3 Hz, 1H), 8.10 (s, 1H), 8.00-7.95 (m, 1H), 7.90 (t, J=7.5 Hz, 1H), 7.86-7.80 (m, 1H), 7.70 (s, 1H), 7.46-7.39 (m, 2H), 6.91-6.84 (m, 1H), 5.10-4.99 (m, 1H), 4.93-4.82 (m, 1H), 4.81-4.64 (m, 2H), 4.49-4.38 (m, 1H), 4.26-3.80 (m, 11H), 3.33 (s, 3H), 3.19-3.09 (m, 1H), 3.00-2.86 (m, 1H), 2.85-2.63 (m, 4H), 2.37-2.28 (m, 2H), 2.05-1.95 (m, 7H), 1.80-1.72 (m, 2H), 1.70-1.58 (m, 4H), 1.54-1.38 (m, 2H).

### Example 204. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-(2-(2,6-dioxopiperidin-3-yl)-1,3-dioxoisoindolin-4-yl)azetidin-3-yl)acetyl)piperidin-4-yl)-2-fluoro-5-methoxybenzamide (Compound 204)

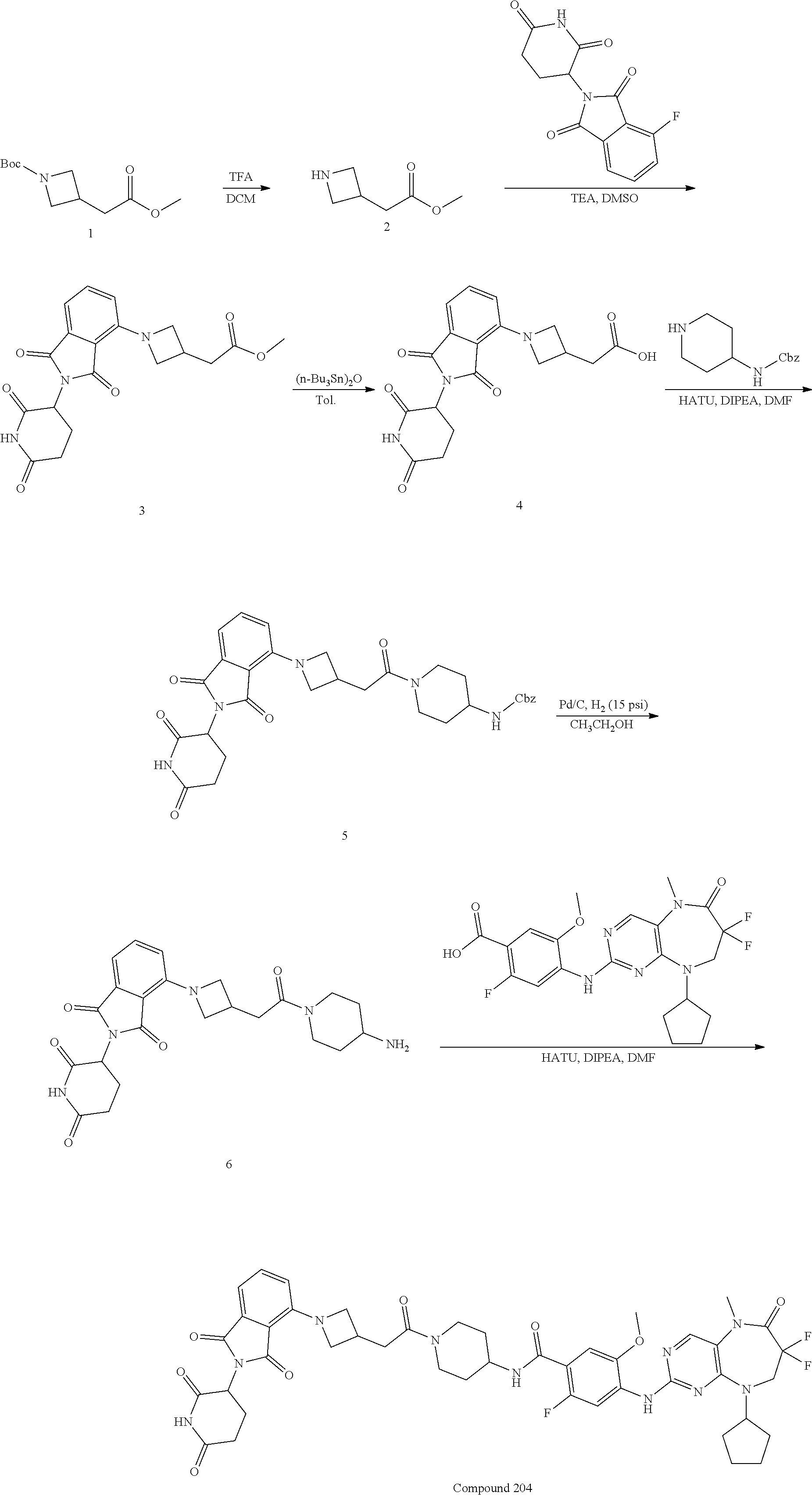

### Step 1. Synthesis of methyl 2-(azetidin-3-yl)acetate (2)

To a solution of tert-butyl 3-(2-methoxy-2-oxoethyl)azetidine-1-carboxylate (840 mg, 3.66 mmol) in DCM (5 mL) was added TFA (1.54 g, 13.51 mmol, 1 mL) at 25° C. The mixture was stirred at 25° C. for 1 hr. TLC (petroleum ether:EtOAc=1:1; Rf=0) showed the starting material was consumed completely and new spots were formed. The reaction mixture was concentrated under reduced pressure to afford methyl 2-(azetidin-3-yl)acetate (890 mg, crude, TFA) as colorless oil, which was used for the next step directly. MS(M+H)⁺=130.2

### Step 2. Synthesis of methyl 2-(1-(2-(2,6-dioxopiperidin-3-yl)-1,3-dioxoisoindolin-4-yl)azetidin-3-yl)acetate (3)

A mixture of 2-(2,6-dioxopiperidin-3-yl)-4-fluoroisoindoline-1,3-dione (909.83 mg, 3.29 mmol), methyl 2-(azetidin-3-yl)acetate (890 mg, 3.66 mmol, TFA salt) and TEA (1.11 g, 10.98 mmol. 1.53 mL) in DMSO (5 mL) was stirred at 100° C. for 12 hours. LCMS showed a main peak with desired mass. The mixture was poured into water (50 mL) and extracted with EtOAc (20 mL×5). The combined organic phase was washed with brine (30 mL×3), dried over Na₂SO₄, filtered and concentrated under reduced pressure to afford methyl 2-(1-(2-(2,6-dioxopiperidin-3-yl)-1,3-dioxoisoindolin-4-yl)azetidin-3-yl)acetate (1.5 g, crude) as a yellow solid, which was used for the next step directly. MS(M+H)⁺=386.1

### Step 3. Synthesis of 2-(1-(2-(2,6-dioxopiperidin-3-yl)-1,3-dioxoisoindolin-4-yl)azetidin-3-yl)acetic acid (4)

A mixture of methyl 2-(1-(2-(2,6-dioxopiperidin-3-yl)-1,3-dioxoisoindolin-4-yl)azetidin-3-yl)acetate (1.5 g, 3.89 mmol) and (n-Bu₃Sn)₂O (9.28 g, 15.57 mmol, 7.93 mL) in toluene (80 mL) was stirred at 110° C. for 16 hr. LCMS showed the starting material was consumed completely, and a main peak with desired mass. The mixture was poured into KF (2.5M, 100 mL) and extracted with EtOAc (30 mL×5). The combined organic phase was washed with brine (20 mL×3), dried over Na₂SO₄, filtered and concentrated under reduced pressure. The residue was purified by flash silica gel chromatography (25 g SepaFlash® Silica Flash Column, Eluent of 0˜100% EtOAc/Petroleum ether gradient @80 mL/min) to afford 2-(1-(2-(2,6-dioxopiperidin-3-yl)-1,3-dioxoisoindolin-4-yl)azetidin-3-yl)acetic acid (1 g, 2.67 mmol, 68.49% yield, 99% purity) as a yellow solid, which was used for the next step directly. MS(M+H)⁺=372.0

### Step 4. Synthesis of benzyl (1-(2-(1-(2-(2,6-dioxopiperidin-3-yl)-1,3-dioxoisoindolin-4-yl)azetidin-3-yl)acetyl)piperidin-4-yl)carbamate (5)

To a solution of 2-(1-(2-(2,6-dioxopiperidin-3-yl)-1,3-dioxoisoindolin-4-yl)azetidin-3-yl)acetic acid (0.5 g, 1.35 mmol) in DMF (5 mL) were added HATU (614.36 mg, 1.62 mmol) and DIPEA (522.05 mg, 4.04 mmol, 703.57 μL). The mixture was stirred at 25° C. for 10 min. Then benzyl piperidin-4-ylcarbamate (378.56 mg, 1.62 mmol) was added and the resulting mixture was stirred at 25° C. for 2 h. LCMS showed a main peak with desired mass. The mixture was poured into water (60 mL) and extracted with EtOAc (20 mL×3). The combined organic phase was washed with brine (10 mL×3), dried over Na₂SO 4, filtered and concentrated under reduced pressure. The residue was purified by flash silica gel chromatography (25 g SepaFlash® Silica Flash Column, Eluent of 0˜100% EtOAc/Petroleum ether gradient @80 mL/min) to afford benzyl (1-(2-(1-(2-(2,6-dioxopiperidin-3-yl)-1,3-dioxoisoindolin-4-yl)azetidin-3-yl)acetyl)piperidin-4-yl)carbamate (0.4 g, 619.45 μmol, 46.01% yield, 91% purity) as a yellow solid, which was used for the next step directly. MS(M+H)⁺=588.3

### Step 5. Synthesis of 4-(3-(2-(4-aminopiperidin-1-yl)-2-oxoethyl)azetidin-1-yl)-2-(2,6-dioxopiperidin-3-yl)isoindoline-1,3-dione (6)

To a solution of benzyl (1-(2-(1-(2-(2,6-dioxopiperidin-3-yl)-1,3-dioxoisoindolin-4-yl)azetidin-3-yl)acetyl)piperidin-4-yl)carbamate (0.1 g, 170.18 μmol) in CF₃CH₂OH (10 mL) was added Pd/C (0.1 g, 170.18 μmol, 10% purity). The mixture was stirred at 25° C. under H₂ (15 Psi) for 4 hr. LCMS showed the starting material was consumed completely and a main peak with desired mass. The reaction mixture was filtered through a Celite pad and the filtrate was concentrated to afford 4-(3-(2-(4-aminopiperidin-1-yl)-2-oxoethyl)azetidin-1-yl)-2-(2,6-dioxopiperidin-3-yl)isoindoline-1,3-dione (80 mg, crude) as a yellow solid, which was used for the next step directly. MS(M+H)⁺=454.2

### Step 6. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-(2-(2,6-dioxopiperidin-3-yl)-1,3-dioxoisoindolin-4-yl)azetidin-3-yl)acetyl)piperidin-4-yl)-2-fluoro-5-methoxybenzamide (Compound 204)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (90 mg, 193.37 μmol) in DMF (3 mL) were added HATU (88.23 mg, 232.05 μmol) and DIPEA (74.97 mg, 580.12 μmol, 101.04 μL). The mixture was stirred at 25° C. for 10 min. Then 4-(3-(2-(4-aminopiperidin-1-yl)-2-oxoethyl)azetidin-1-yl)-2-(2,6-dioxopiperidin-3-yl)isoindoline-1,3-dione (78.92 mg, 174.03 μmol) was added and the resulting mixture was stirred at 25° C. for 2 h. LCMS showed a main peak with desired mass. The mixture was diluted with EtOAc (20 mL) and washed with brine (6 mL). The organic phase was dried over Na₂SO₄ and concentrated under reduced pressure. The residue was purified by flash silica gel chromatography (4 g SepaFlash® Silica Flash Column, Eluent of 0˜100% EtOAc/Petroleum ether gradient @60 mL/min; Eluent of 0˜50% Methanol/EtOAc @60 mL/min) and re-purified by prep-HPLC (column: Waters Xbridge 150×25 mm×5 μm; mobile phase: [water(NH₄HCO₃)-ACN]; B %: 40%-70%, 9 min) and prep-TLC (dichloromethane:methanol=10:1; R_{f}=0.5). The impure product was further purified by prep-HPLC (column: Waters Xbridge 150×25 mm×5 μm; mobile phase: [water(NH₄HCO₃)-ACN]; B %: 41%-71%, 8 min), and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-(2-(2,6-dioxopiperidin-3-yl)-1,3-dioxoisoindolin-4-yl)azetidin-3-yl)acetyl)piperidin-4-yl)-2-fluoro-5-methoxybenzamide (18.2 mg, 19.80 μmol, 43.50% yield, 98% purity) as a yellow solid. MS(M+H)⁺=901.2

¹H NMR (400 MHz, DMSO-d₆) δ=11.24-10.84 (m, 1H), 8.33-8.20 (m, 2H), 8.04 (s, 1H), 7.97 (br dd, J=2.8, 7.4 Hz, 1H), 7.58-7.51 (m, 1H), 7.20 (d, J=6.8 Hz, 1H), 7.09 (d, J=6.9 Hz, 1H), 6.76 (d, J=8.5 Hz, 1H), 5.03 (dd, J=5.4, 12.8 Hz, 1H), 4.89-4.75 (m, 1H), 4.40-4.25 (m, 3H), 4.13-3.96 (m, 3H), 3.91 (s, 3H), 3.89-3.74 (m, 3H), 3.33 (br s, 3H), 3.19-3.10 (m, 1H), 2.98-2.71 (m, 5H), 2.62-2.54 (m, 2H), 2.05-1.91 (m, 3H), 1.90-1.78 (m, 2H), 1.76-1.57 (m, 6H), 1.54-1.33 (m, 2H).

### Example 205. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(2-(1-(2-(2,6-dioxopiperidin-3-yl)-1,3-dioxoisoindolin-5-yl)azetidin-3-yl)acetyl)piperidin-4-yl)-2-fluoro-5-methoxybenzamide (Compound 205)

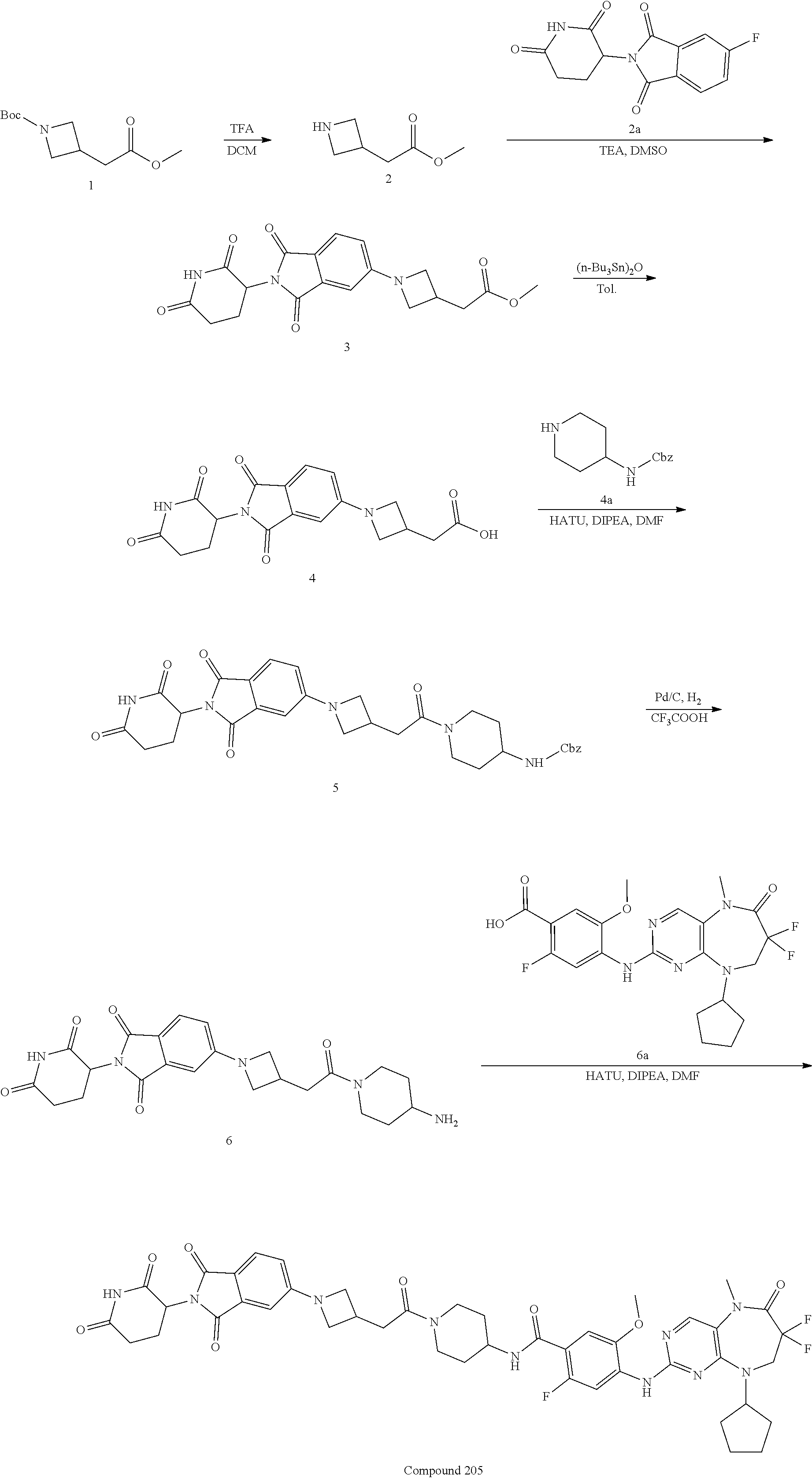

The compound 205 was synthesized by the method described in the scheme similar to the method described in Example 204.

MS(M+H)⁺=901.2, ¹H NMR (400 MHz, DMSO-d₆) δ=11.06 (s, 1H), 8.30 (s, 1H), 8.25 (d, J=13.4 Hz, 1H), 8.04 (s, 1H), 7.96 (dd, J=2.9, 7.7 Hz, 1H), 7.62 (d, J=8.3 Hz, 1H), 7.20 (d, J=6.6 Hz, 1H), 6.76 (d, J=1.8 Hz, 1H), 6.63 (dd, J=1.9, 8.4 Hz, 1H), 5.05 (dd, J=5.3, 12.8 Hz, 1H), 4.87-4.76 (m, 1H), 4.30 (d, J=12.9 Hz, 1H), 4.18 (t, J=8.2 Hz, 2H), 4.13-3.97 (m, 3H), 3.91 (s, 3H), 3.86 (d, J=14.1 Hz, 1H), 3.72-3.63 (m, 2H), 3.30 (s, 3H), 3.19-3.09 (m, 1H), 3.09-2.99 (m, 1H), 2.95-2.70 (m, 4H), 2.62-2.53 (m, 2H), 2.08-1.91 (m, 3H), 1.91-1.78 (m, 2H), 1.76-1.56 (m, 6H), 1.55-1.32 (in, 2H).

### Example 206. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-((2S,4S)-4-((2-(2,6-dioxopiperidin-3-yl)-1,3-dioxoisoindolin-4-yl)amino)pyrrolidine-2-carbonyl)piperidin-4-yl)-2-fluoro-5-methoxybenzamide (Compound 206)

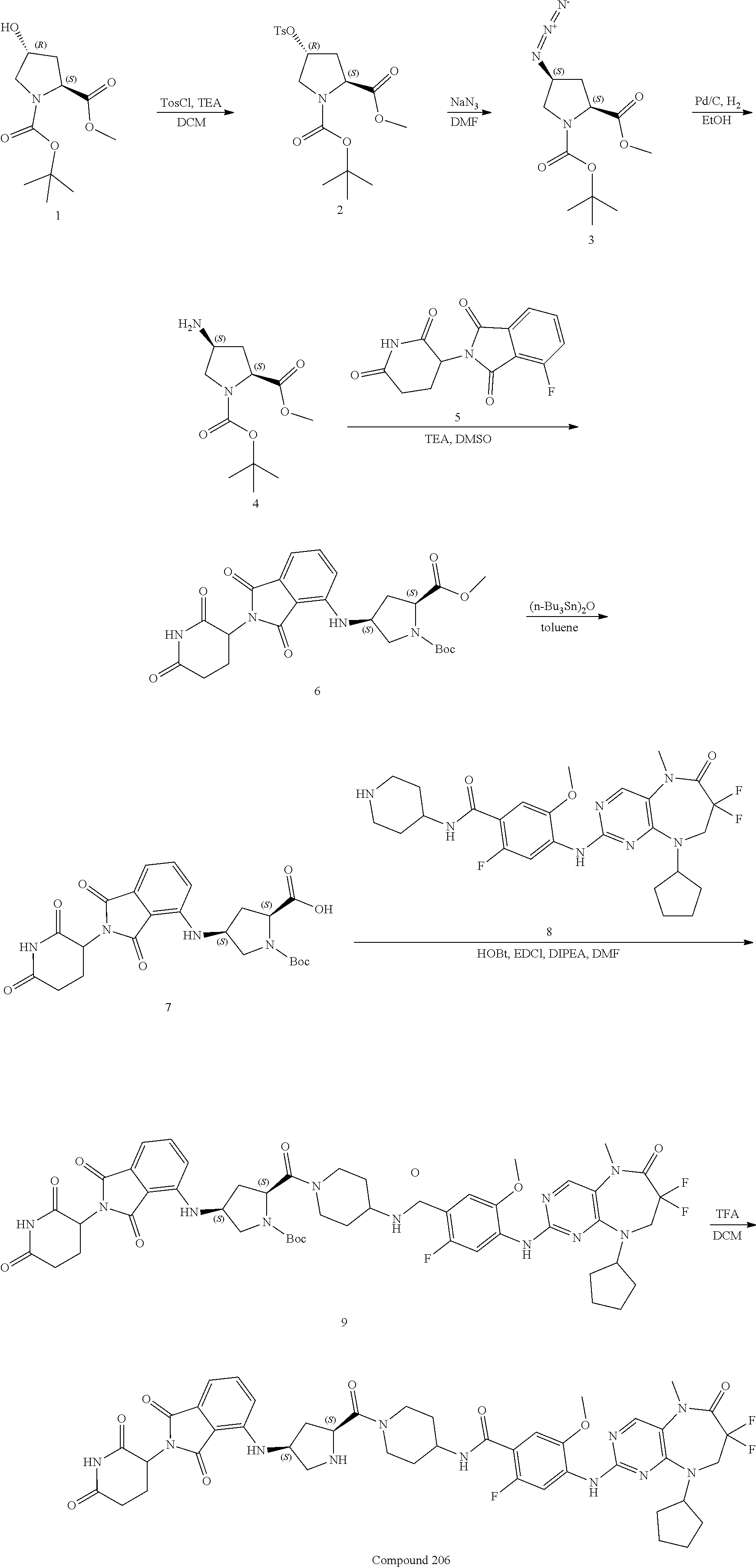

### Step 1 Synthesis oft)(tert-butyl) 2-methyl (2S,4R)-4-(tosyloxy)pyrrolidine-1,2-dicarboxylate (2)

To a solution of 1-(tert-butyl) 2-methyl (2S,4R)-4-hydroxypyrrolidine-1,2-dicarboxylate (5 g, 20.39 mmol) in DCM (60 mL) were added TEA (6.19 g, 61.16 mmol, 8.51 mL) and TosCl (5.05 g, 26.50 mmol) at 20° C. and the resulting mixture was stirred at 20° C. for 16 h. LCMS showed 33% of starting material remained and a peak (20%) with desired mass. The resulting mixture was stirred at 20° C. for 16 h. LCMS showed starting material was consumed completely and a peak (65%) with desired mass. The reaction mixture was concentrated in vacuum. The residue was purified by flash silica gel chromatography (40 g SepaFlash® Silica Flash Column, Eluent of 0˜18% EtOAc/Petroleum ether gradient @100 mL/min) to afford 1-(tert-butyl) 2-methyl (2S,4R)-4-(tosyloxy)pyrrolidine-1,2-dicarboxylate (4.7 g, 11.53 mmol, 56.56% yield, 98% purity) as a yellow oil. MS(M−100+H)⁺300.3

### Step 2. Synthesis of 1-(tert-butyl) 2-methyl (2S,4S)-4-azidopyrrolidine-1,2-dicarboxylate (3)

To a solution of 1-(tert-butyl) 2-methyl (2S,4R)-4-(tosyloxy)pyrrolidine-1,2-dicarboxylate (4.7 g, 11.77 mmol) in DMF (50 mL) was added NaN₃ (1.53 g, 23.53 mmol) at 20° C. and the resulting mixture was stirred at 70° C. for 16 h. LCMS showed starting material was consumed completely and 93% peak with desired mass was detected. The organic layer was diluted with H₂O (150 mL) and extracted with EtOAc (150 mL×3). The organic layer was washed with brine (150 mL), dried over Na₂SO₄, filtered and concentrated to afford 1-(tert-butyl) 2-methyl (2S,4S)-4-azidopyrrolidine-1,2-dicarboxylate (5.3 g, crude) as a yellow oil. MS(M−N₃−55)⁺=171.5

### Step 3. Synthesis of 1-(tert-butyl) 2-methyl (2S,4S)-4-aminopyrrolidine-1,2-dicarboxylate (4)

To a solution of 1-(tert-butyl) 2-methyl (2S,4S)-4-azidopyrrolidine-1,2-dicarboxylate (5.3 g, 19.61 mmol) in CF₃CH₂OH (60 mL) was added Pd/C (1 g, 10% purity) under N₂ atmosphere. The suspension was degassed and purged with H₂ for 3 times. The mixture was stirred under H₂ (15 Psi) at 20° C. for 16 h. LCMS showed starting material was consumed completely and peak with desired mass was detected. The reaction mixture was diluted with EtOH (200 mL) and filtered. The filtrate was concentrated to afford 1-(tert-butyl) 2-methyl (2S,4S)-4-aminopyrrolidine-1,2-dicarboxylate (4.9 g, crude) as a yellow oil. MS(M−56+H)⁺=189.5

### Step 4. Synthesis of 1-(tert-butyl) 2-methyl (2S,4S)-4-((2-(2,6-dioxopiperidin-3-yl)-1,3-dioxoisoindolin-4-yl)amino)pyrrolidine-1,2-dicarboxylate (6)

To a solution of 2-(2,6-dioxo-3-piperidyl)-4-fluoro-isoindoline-1,3-dione (800 mg, 2.90 mmol) in DMSO (12 mL) were added TEA (879.20 mg, 8.69 mmol, 1.21 mL) and 1-(tert-butyl) 2-methyl (2S,4S)-4-aminopyrrolidine-1,2-dicarboxylate (1.42 g, 5.79 mmol) at 20° C. and the resulting mixture was stirred at 100° C. for 16 h. LCMS showed starting material was consumed completely and 47% peak with desired mass was detected. The reaction mixture was diluted with H₂O (40 mL) and extracted with EtOAc (40 mL×3). The organic layer was washed with brine (40 mL×3), dried over Na₂SO₄, filtered and concentrated. The residue was purified by flash silica gel chromatography (20 g SepaFlash® Silica Flash Column, Eluent of 0˜55% EtOAc/Petroleum ether gradient @100 mL/min) to afford 1-(tert-butyl) 2-methyl (2S,4S)-4-((2-(2,6-dioxopiperidin-3-yl)-1,3-dioxoisoindolin-4-yl)amino)pyrrolidine-1,2-dicarboxylate (914 mg, 1.64 mmol, 56.75% yield, 90% purity) as a yellow solid. MS(M−100+H)⁺=401.2

### Step 5. Synthesis of (2S,4S)-1-(tert-butoxycarbonyl)-4-((2-(2,6-dioxopiperidin-3-yl)-1,3-dioxoisoindolin-4-yl)amino)pyrrolidine-2-carboxylic acid (7)

To a solution of 1-(tert-butyl) 2-methyl (2S,4S)-4-((2-(2,6-dioxopiperidin-3-yl)-1,3-dioxoisoindolin-4-yl)amino)pyrrolidine-1,2-dicarboxylate (400 mg, 799.20 μmol) in toluene (10 mL) was added tributyl (tributylstannyloxy) stannane (1.91 g, 3.20 mmol, 1.63 mL) at 20° C. and the resulting mixture was stirred at 110° C. for 16 h. LCMS showed starting material was consumed completely and 79% peak with desired mass was detected. The reaction mixture was concentrated in vacuum to afford (2S,4S)-1-(tert-butoxycarbonyl)-4-((2-(2,6-dioxopiperidin-3-yl)-1,3-dioxoisoindolin-4-yl)amino)pyrrolidine-2-carboxylic acid (389 mg, crude) as a yellow oil. MS(M−100+H)⁺=387.3

### Step 6. Synthesis of tert-butyl (2S,4S)-2-(4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzamido)piperidine-1-carbonyl)-4-((2-(2,6-dioxopiperidin-3-yl)-1,3-dioxoisoindolin-4-yl)amino)pyrrolidine-1-carboxylate (9)

To a solution of (2S,4S)-1-(tert-butoxycarbonyl)-4-((2-(2,6-dioxopiperidin-3-yl)-1,3-dioxoisoindolin-4-yl)amino)pyrrolidine-2-carboxylic acid (239 mg, 491.29 μmol) and 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxy-N-(piperidin-4-yl)benzamide (286.93 mg, 491.29 μmol, HCl) in DMF (8 mL) were added EDCI (141.27 mg, 736.94 μmol), HOBt (99.58 mg, 736.94 μmol) and DIPEA (190.49 mg, 1.47 mmol, 256.72 μL) at 20° C. and the resulting mixture was stirred at 20° C. for 16 h. LCMS showed all starting material was consumed completely and 60% peak with desired mass was detected. The reaction mixture was diluted with H₂O (25 mL) and extracted with EtOAc (25 mL×3). The organic layer was washed with brine (25 mL×3), dried over Na₂SO₄, filtered and concentrated. The residue was purified by flash silica gel chromatography (12 g SepaFlash® Silica Flash Column, Eluent of 0˜100% EtOAc/Petroleum ether gradient @100 mL/min) to afford tert-butyl (2S,4S)-2-(4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzamido)piperidine-1-carbonyl)-4-((2-(2,6-dioxopiperidin-3-yl)-1,3-dioxoisoindolin-4-yl)amino)pyrrolidine-1-carboxylate (367 mg, 361.21 μmol, 73.52% yield) as a yellow solid. MS(M−100+H)⁺=916.1

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-((2S,4S)-4-((2-(2,6-dioxopiperidin-3-yl)-1,3-dioxoisoindolin-4-yl)amino)pyrrolidine-2-carbonyl)piperidin-4-yl)-2-fluoro-5-methoxybenzamide (Compound 206)

To a solution of tert-butyl (2S,4S)-2-(4-(4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzamido)piperidine-1-carbonyl)-4-((2-(2,6-dioxopiperidin-3-yl)-1,3-dioxoisoindolin-4-yl)amino)pyrrolidine-1-carboxylate (200 mg, 196.84 μmol) in DCM (2 mL) was added TFA (67.33 mg, 590.53 μmol, 43.72 μL) at 20° C. and the resulting mixture was stirred at 20° C. for 1 h. LCMS showed starting material was consumed completely and 82% peak with desired mass was detected. The reaction mixture was concentrated in vacuum. The residue was purified by prep-HPLC (column: Phenomenex luna C18 150*25 mm*10 μm; mobile phase: [water(TFA)-ACN]; B %: 19%-49%, 10 min) and re-purified by prep-HPLC (column: Waters Xbridge 150*25 mm*5 μm; mobile phase: [water(NH₄HCO₃)-ACN]; B %: 39%-69%, 8 min) and lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-((2S,4S)-4-((2-(2,6-dioxopiperidin-3-yl)-1,3-dioxoisoindolin-4-yl)amino)pyrrolidine-2-carbonyl)piperidin-4-yl)-2-fluoro-5-methoxybenzamide (44.2 mg, 47.29 μmol, 24.03% yield, 98% purity) as a yellow solid. SFC (retention time: peak 1=1.996, peak 2=3.296; method: Column: Chiralpak AS-3 50×4.6 mm I. D., 3 μm; Mobile phase: Phase A for CO₂, and Phase B for IPA+ACN (0.05% DEA); Gradient elution: 40% IPA+ACN (0.05% DEA) in CO₂; Flow rate: 3 mL/min; Detector: PDAColumn Temp: 35 C; Back Pressure: 100 Bar). MS(M+H)⁺=916.1

¹H NMR (400 MHz, DMSO-d₆) δ=11.09 (br s, 1H), 8.31-8.21 (m, 2H), 8.04 (s, 1H), 8.02-7.90 (m, 1H), 7.64-7.55 (m, 1H), 7.20 (d, J=6.5 Hz, 1H), 7.13 (dd, J=5.0, 8.6 Hz, 1H), 7.05 (d, J=7.1 Hz, 1H), 6.53 (dd, J=7.6, 12.2 Hz, 1H), 5.05 (dd, J=5.3, 12.6 Hz, 1H), 4.87-4.76 (m, 1H), 4.32 (d, J=12.7 Hz, 1H), 4.24-4.14 (m, 1H), 4.13-3.95 (m, 5H), 3.91 (s, 3H), 3.33 (s, 3H), 3.27-3.10 (m, 2H), 3.02 (t, J=10.8 Hz, 1H), 2.94-2.84 (m, 2H), 2.84-2.75 (m, 1H), 2.63-2.55 (m, 2H), 2.06-1.92 (m, 3H), 1.91-1.80 (m, 2H), 1.78-1.29 (m, 10H).

### Example 207. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-((2R,4S)-4-((2-(2,6-dioxopiperidin-3-yl)-1,3-dioxoisoindolin-4-yl)amino)pyrrolidine-2-carbonyl)piperidin-4-yl)-2-fluoro-5-methoxybenzamide (Compound 207)

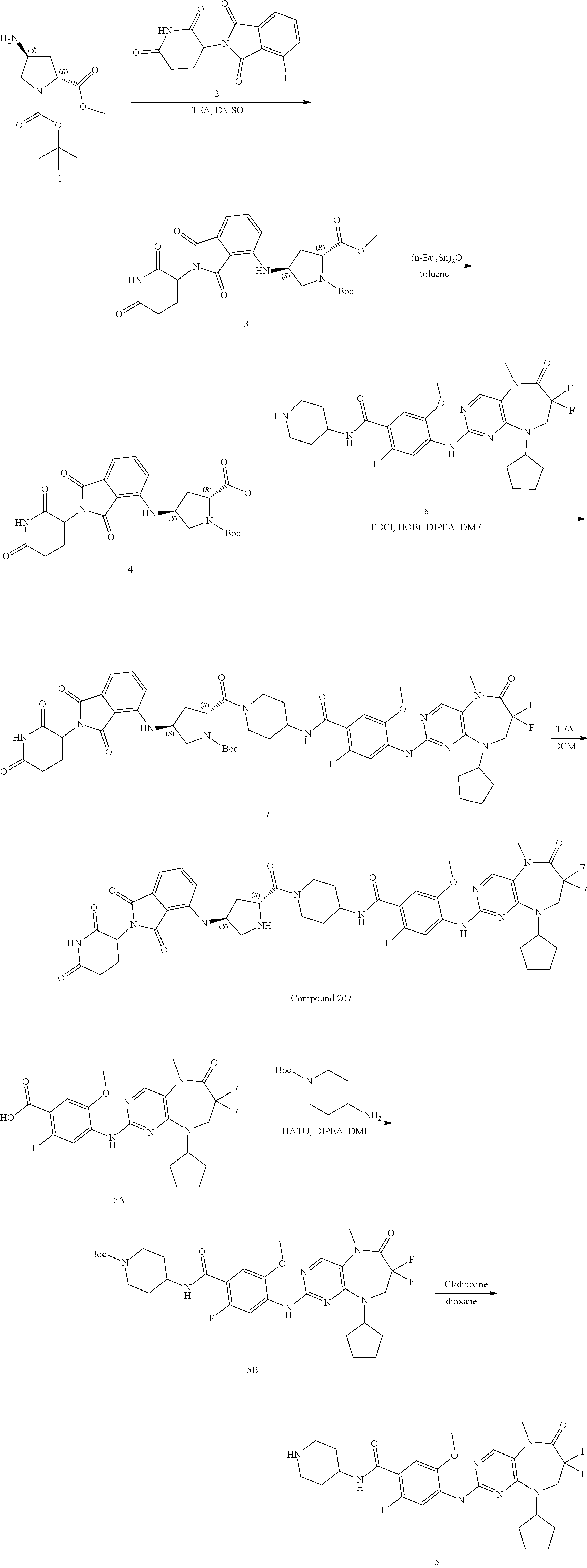

The compound 207 was synthesized by the method described in the scheme similar to the method described in Example 206.

MS(M+H)⁺=916.2, ¹H NMR (400 MHz, DMSO-d₆) δ=11.10 (br s, 1H), 8.33-8.19 (m, 2H), 8.07-7.90 (m, 2H), 7.60 (t, J=7.9 Hz, 1H), 7.20 (d, J=4.8 Hz, 1H), 7.14 (d, J=8.6 Hz, 1H), 7.07 (d, J=7.0 Hz, 1H), 6.45 (t, J=7.4 Hz, 1H), 5.05 (dd, J=5.4, 12.7 Hz, 1H), 4.87-4.76 (m, 1H), 4.38-4.26 (m, 1H), 4.19-3.98 (m, 5H), 3.91 (s, 4H), 3.33 (s, 3H), 3.24-3.00 (m, 2H), 2.93-2.75 (m, 2H), 2.66-2.53 (m, 4H), 2.10 (dd, J=3.9, 5.7 Hz, 1H), 2.07-1.91 (m, 4H), 1.91-1.79 (m, 2H), 1.78-1.55 (m, 6H), 1.54-1.35 (m, 2H).

### Example 208. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(7-(2-(2,6-dioxopiperidin-3-yl)-1,3-dioxoisoindolin-4-yl)-7-azaspiro[3.5]nonan-2-yl)piperidin-4-yl)-2-fluoro-5-methoxybenzamide (Compound 208)

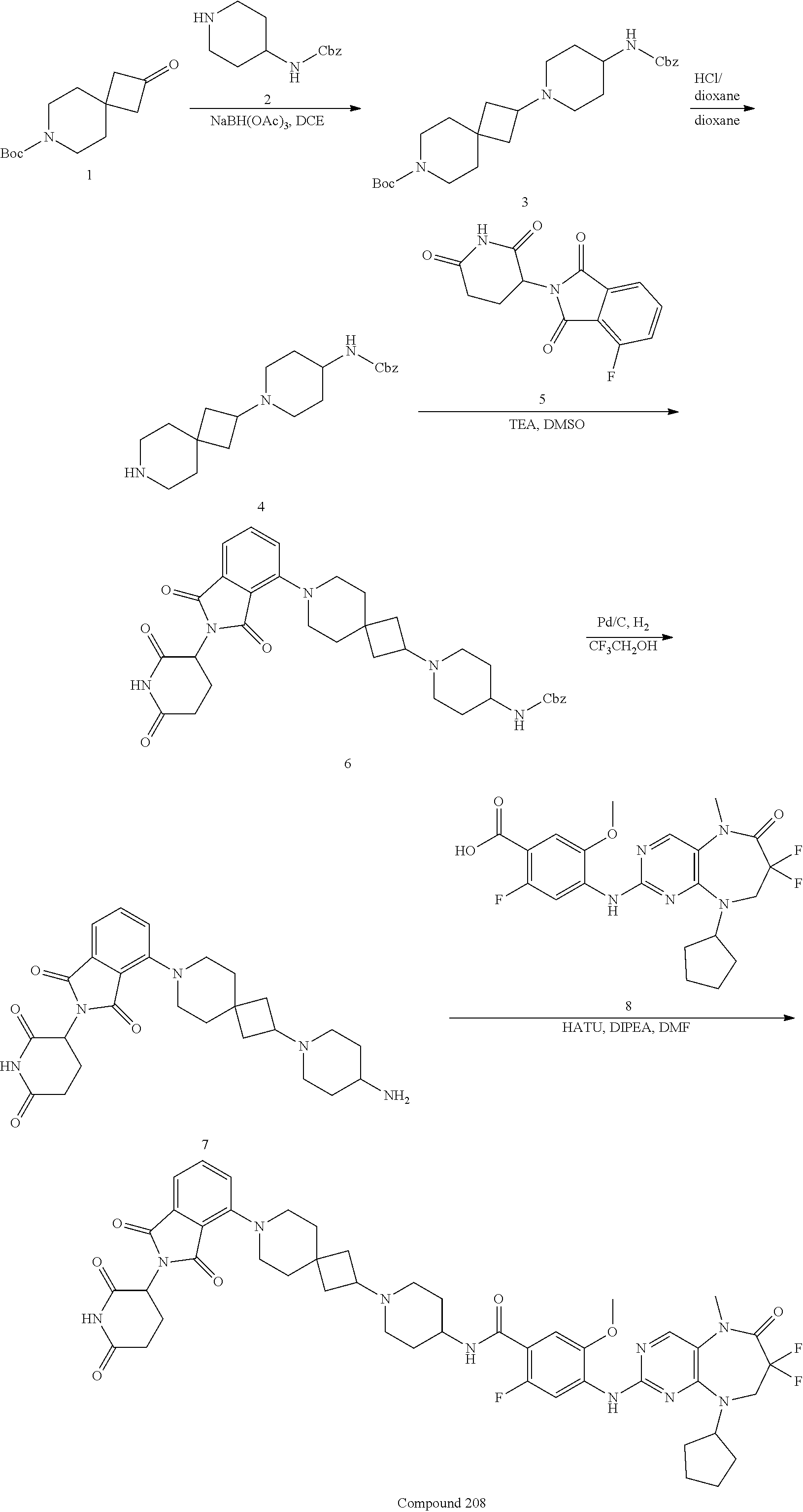

### Step 1. Synthesis of tert-butyl 2-(4-(((benzyloxy)carbonyl)amino)piperidin-1-yl)-7-azaspiro[3.5]nonane-7-carboxylate (3)

To a solution of tert-butyl 2-oxo-7-azaspiro[3.5]nonane-7-carboxylate (0.5 g, 2.09 mmol) in DCE (10 mL) was added benzyl piperidin-4-ylcarbamate (538.47 mg, 2.30 mmol) at 25° C. After stirring for 0.5 h, NaBH(OAc)₃ (664.23 mg, 3.13 mmol) was added and the resulting mixture was stirred at 25° C. for 12 h. LCMS showed ˜97% of desired mass was detected. The reaction mixture was diluted with water (50 mL) and extracted with DCM (50 mL×3). The combined organic layers were washed with brine (100 mL), dried over anhydrous sodium sulfate, filtered and concentrated under reduced pressure to afford tert-butyl 2-(4-(((benzyloxy)carbonyl)amino)piperidin-1-yl)-7-azaspiro[3.5]nonane-7-carboxylate (960 mg, crude) as a white solid. MS(M+H)⁺=458.2.

### Step 2. Synthesis of benzyl (1-(7-azaspiro[3.5]nonan-2-yl)piperidin-4-yl)carbamate (4)

To a solution of tert-butyl 2-(4-(((benzyloxy)carbonyl)amino)piperidin-1-yl)-7-azaspiro[3.5]nonane-7-carboxylate (960 mg, 2.10 mmol) in dioxane (5 mL) was added HCl/dioxane (4 M, 5 mL) at 25° C. The mixture was stirred at 25° C. for 1 h. LCMS showed starting material was consumed. The mixture was concentrated under reduced pressure to afford benzyl (1-(7-azaspiro[3.5]nonan-2-yl)piperidin-4-yl)carbamate (1.2 g, crude, HCl salt) as a white solid. MS(M+H)⁺=358.3.

### Step 3. Synthesis of benzyl (1-(7-(2-(2,6-dioxopiperidin-3-yl)-1,3-dioxoisoindolin-4-yl)-7-azaspiro[3.5]nonan-2-yl)piperidin-4-yl)carbamate (6)

To a solution of benzyl (1-(7-azaspiro[3.5]nonan-2-yl)piperidin-4-yl)carbamate (100 mg, 253.84 μmol, HCl salt) in DMSO (2 mL) were added TEA (102.74 mg, 1.02 mmol, 141.32 μL) and 2-(2,6-dioxopiperidin-3-yl)-4-fluoroisoindoline-1,3-dione (73.62 mg, 266.53 μmol) at 25° C. The mixture was stirred at 90° C. for 4 h. LCMS showed ˜90% of desired mass was detected. The residue was purified by prep-HPLC (column: Shim-pack C18 150×25×10 μm; mobile phase: [water(FA)-ACN]; B %: 10%-40%, 10 min) followed by lyophilization to afford benzyl (1-(7-(2-(2,6-dioxopiperidin-3-yl)-1,3-dioxoisoindolin-4-yl)-7-azaspiro[3.5]nonan-2-yl)piperidin-4-yl)carbamate (120 mg, 176.37 μmol, 34.74% yield, 90.2% purity) as a yellow solid. MS(M+H)⁺=614.3.

### Step 4. Synthesis of 4-(2-(4-aminopiperidin-1-yl)-7-azaspiro[3.5]nonan-7-yl)-2-(2,6-dioxopiperidin-3-yl)isoindoline-1,3-dione (7)

To a solution of benzyl (1-(7-(2-(2,6-dioxopiperidin-3-yl)-1,3-dioxoisoindolin-4-yl)-7-azaspiro[3.5]nonan-2-yl)piperidin-4-yl)carbamate (120 mg, 195.53 μmol) in CF₃CH₂OH (5 mL) was added Pd/C (30 mg, 39.11 μmol, 10% purity) under N2 atmosphere at 25° C. The mixture was stirred at 25° C. for 3 h under H₂ atmosphere (15 PSI). LCMS showed ˜83% of desired mass was detected. The mixture was filtered. The filtrate was concentrated under reduced pressure to afford 4-(2-(4-aminopiperidin-1-yl)-7-azaspiro[3.5]nonan-7-yl)-2-(2,6-dioxopiperidin-3-yl)isoindoline-1,3-dione (60 mg, 99.71 μmol, 51.00% yield, 79.7% purity) as yellow oil. MS(M+H)⁺=480.3

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(1-(7-(2-(2,6-dioxopiperidin-3-yl)-1,3-dioxoisoindolin-4-yl)-7-azaspiro[3.5]nonan-2-yl)piperidin-4-yl)-2-fluoro-5-methoxybenzamide (Compound 208)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (58.23 mg, 125.11 μmol) in DMF (2 mL) were added HATU (118.93 mg, 312.78 μmol) and DIEA (48.51 mg, 375.34 μmol, 65.38 μL) at 25° C., after stirring for 0.5 h, then 4-(2-(4-aminopiperidin-1-yl)-7-azaspiro[3.5]nonan-7-yl)-2-(2,6-dioxopiperidin-3-yl)isoindoline-1,3-dione (60 mg, 125.11 μmol) was added and the resulting mixture was stirred at 25° C. for 12 h. LCMS showed ˜60% of desired mass was detected. The reaction mixture was diluted with water (50 mL) and extracted with EtOAc (50 mL×4). The combined organic layers were washed with brine (50 mL), dried over anhydrous sodium sulfate, filtered and concentrated under reduced pressure. The residue was purified by prep-HPLC (column: YMC Triart 30×150 mm×7 μm; mobile phase: [water(HCl)-ACN]; B %: 34%-54%, 7 min) and prep-HPLC (column: Waters Xbridge 150×25 mm×5 μm; mobile phase: [water(NH₄HCO₃)-ACN]; B %: 57%-87%, 10 min) followed by lyophilization to afford 4-((9-cyclopentyl-7,7-difluoro-5-methyl-6-oxo-6,7,8,9-tetrahydro-5H-pyrimido[4,5-b][1,4]diazepin-2-yl)amino)-N-(1-(7-(2-(2,6-dioxopiperidin-3-yl)-1,3-dioxoisoindolin-4-yl)-7-azaspiro[3.5]nonan-2-yl)piperidin-4-yl)-2-fluoro-5-methoxybenzamide (9 mg, 9.42 μmol, 7.53% yield, 97% purity) as a yellow solid. MS(M+H)⁺=927.4.

¹H NMR (400 MHz, DMSO-d₆) δ=11.08 (br. s, 1H), 8.29 (s, 1H), 8.24 (d, J=13.2 Hz, 1H), 8.03 (s, 1H), 7.90-7.87 (m, 1H), 7.68-7.65 (m, 1H), 7.32 (t, J=7.6 Hz, 2H), 7.18 (d, J=6.8 Hz, 1H), 5.10-5.06 (dd, J=5.2, 12.8 Hz, 1H), 4.84-4.80 (m, 1H), 4.07 (t, J=14 Hz, 2H), 3.91 (s, 3H), 3.75-3.72 (m, 1H), 3.30 (s, 3H), 3.25-3.15 (m, 5H), 2.88-2.69 (m, 4H), 2.61-2.56 (m, 3H), 2.02-1.95 (m, 3H), 1.82-1.72 (m, 8H), 1.64-1.48 (m, 10H).

### Example 209. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(7-(2-(2,6-dioxopiperidin-3-yl)-1,3-dioxoisoindolin-4-yl)-2,7-diazaspiro[3.5]nonan-2-yl)piperidin-1-yl)-2-fluoro-5-methoxybenzamide (Compound 209)

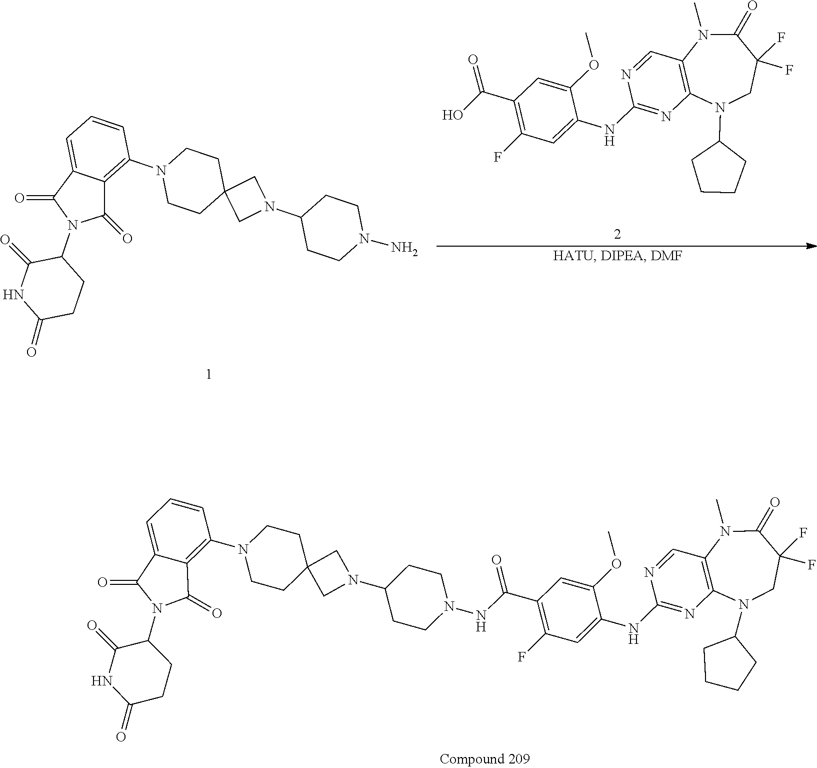 [SMILES: NN1CCC(N2CC3(CCN(c4cccc5c4C(=O)N(C4CCC(=O)NC4=O)C5=O)CC3)C2)CC1 ; COc1cc(C(=O)O)c(F)cc1Nc1ncc2c(n1)N(C1CCCC1)CC(F)(F)C(=O)N2C ; "HATU, DIPEA, DMF" ; COc1cc(C(=O)NN2CCC(N3CC4(CCN(c5cccc6c5C(=O)N(C5CCC(=O)NC5=O)C6=O)CC4)C3)CC2)c(F)cc1Nc1ncc2c(n1)N(C1CCCC1)CC(F)(F)C(=O)N2C]

To a solution of 4-((9-cyclopentyl-7,7-difluoro-5-methyl-6-oxo-6,7,8,9-tetrahydro-5H-pyrimido[4,5-b][1,4]diazepin-2-yl)amino)-2-fluoro-5-methoxybenzoic acid (440 mg, 945.37 μmol) in DMF (3 mL) were added HATU (395.41 mg, 1.04 mmol) and DIPEA (366.55 mg, 2.84 mmol, 494.00 μL). The mixture was stirred at 20° C. for 10 min and a solution of 4-(2-(1-aminopiperidin-4-yl)-2,7-diazaspiro[3.5]nonan-7-yl)-2-(2,6-dioxopiperidin-3-yl)isoindoline-1,3-dione (545.17 mg, 1.13 mmol) in DMF (3 mL) was added and the resulting mixture was stirred at 20° C. for 1 h. LCMS showed all starting material was consumed completely and a peak (38%) with desired mass. The reaction mixture was diluted with H₂O (15 mL) and extracted with EtOAc (15 mL×3). The combined organic layer was washed with brine (15 mL×3), dried over Na₂SO₄, filtered and concentrated. The residue was purified by flash silica gel chromatography (10 g SepaFlash® Silica Flash Column, Eluent of 0˜100% EtOAc/Petroleum ether 0˜50% Dichloromethane/Methanol gradient @100 mL/min) followed by prep-HPLC (column: Phenomenex luna C18 150*40 mm*15 um; mobile phase: [water(TFA)-ACN]; B %: 20%-50%, 10 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(7-(2-(2,6-dioxopiperidin-3-yl)-1,3-dioxoisoindolin-4-yl)-2,7-diazaspiro[3.5]nonan-2-yl)piperidin-1-yl)-2-fluoro-5-methoxybenzamide (207.9 mg, 189.55 μmol, 20.05% yield, 95% purity, 2 TFA) as a yellow solid. MS(M+H)⁺=928.1

¹H NMR (400 MHz, DMSO-d₆) δ=11.10 (s, 1H), 10.06-9.91 (m, 1H), 9.27 (s, 1H), 8.30 (s, 1H), 8.24 (br d, J=13.0 Hz, 1H), 8.13 (s, 1H), 7.74-7.66 (m, 1H), 7.42-7.29 (m, 2H), 7.14 (d, J=6.4 Hz, 1H), 5.14-5.05 (m, 1H), 4.83 (td, J=8.2, 16.2 Hz, 1H), 4.15-3.96 (m, 6H), 3.91 (s, 3H), 3.39-3.22 (m, 6H), 3.19 (br s, 2H), 3.11 (br d, J=9.5 Hz, 2H), 2.95-2.83 (m, 1H), 2.76 (br t, J=9.7 Hz, 2H), 2.59 (br d, J=15.9 Hz, 2H), 2.19-1.81 (m, 9H), 1.79-1.69 (m, 2H), 1.62-1.41 (m, 6H).

### Example 210. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(2-(2,6-dioxopiperidin-3-yl)-1,3-dioxoisoindolin-4-yl)-2,7-diazaspiro[3.5]nonan-2-yl)piperidin-1-yl)-2-fluoro-5-methoxybenzamide (Compound 210)

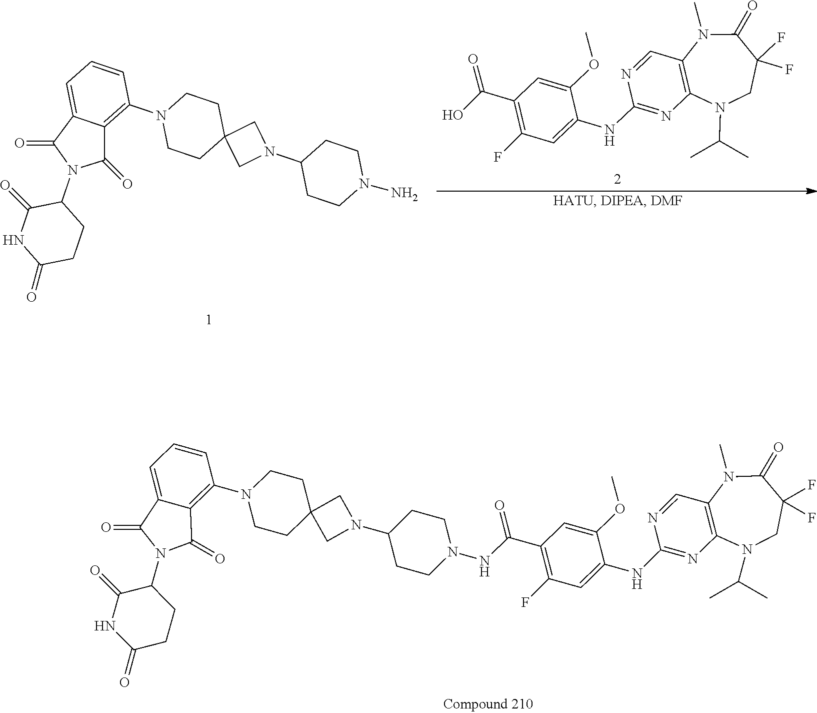 [SMILES: NN1CCC(N2CC3(CCN(c4cccc5c4C(=O)N(C4CCC(=O)NC4=O)C5=O)CC3)C2)CC1 ; COc1cc(C(=O)O)c(F)cc1Nc1ncc2c(n1)N(C(C)C)CC(F)(F)C(=O)N2C ; "HATU, DIPEA, DMF" ; COc1cc(C(=O)NN2CCC(N3CC4(CCN(c5cccc6c5C(=O)N(C5CCC(=O)NC5=O)C6=O)CC4)C3)CC2)c(F)cc1Nc1ncc2c(n1)N(C(C)C)CC(F)(F)C(=O)N2C]

To a solution of 4-((7,7-difluoro-9-isopropyl-5-methyl-6-oxo-6,7,8,9-tetrahydro-5H-pyrimido[4,5-b][1,4]diazepin-2-yl)amino)-2-fluoro-5-methoxybenzoic acid (420 mg, 955.88 μmol) in DMF (3 mL) were added HATU (399.80 mg, 1.05 mmol) and DIPEA (370.62 mg, 2.87 mmol, 499.49 μL). The mixture was stirred at 20° C. for 10 min and a solution of 4-(2-(1-aminopiperidin-4-yl)-2,7-diazaspiro[3.5]nonan-7-yl)-2-(2,6-dioxopiperidin-3-yl)isoindoline-1,3-dione (551.22 mg, 1.15 mmol) in DMF (3 mL) was added and the resulting mixture was stirred at 20° C. for 1 h. LCMS showed all starting material was consumed completely and a peak (46%) with desired mass. The reaction mixture was diluted with H₂O (15 mL) and extracted with EtOAc (15 mL×3). The combined organic layer was washed with brine (15 mL×3), dried over Na₂SO₄, filtered and concentrated. The residue was purified by flash silica gel chromatography (10 g SepaFlash® Silica Flash Column, Eluent of 0˜100% EtOAc/Petroleum ether 0˜50% Dichloromethane/Methanol gradient @100 mL/min) followed by prep-HPLC (column: Phenomenex luna C18 150*40 mm*15 um; mobile phase: [water(TFA)-ACN]; B %: 16%-46%, 10 min) and the eluent was lyophilized to afford 4-((7,7-difluoro-9-isopropyl-5-methyl-6-oxo-6,7,8,9-tetrahydro-5H-pyrimido[4,5-b][1, 4]diazepin-2-yl)amino)-N-(4-(7-(2-(2,6-dioxopiperidin-3-yl)-1,3-dioxoisoindolin-4-yl)-2,7-diazaspiro[3.5]nonan-2-yl)piperidin-1-yl)-2-fluoro-5-methoxybenzamide (146.7 mg, 138.62 μmol, 14.50% yield, 96% purity, 2 TFA) as a yellow solid. MS(M+H)⁺=902.1

¹H NMR (400 MHz, DMSO-d₆) δ=11.11 (s, 1H), 10.05-9.92 (m, 1H), 9.25 (s, 1H), 8.32-8.22 (m, 2H), 8.08 (s, 1H), 7.75-7.67 (m, 1H), 7.41-7.32 (m, 2H), 7.19-7.11 (m, 1H), 5.11 (br dd, J=5.6, 12.7 Hz, 1H), 4.93-4.86 (m, 1H), 4.12-4.05 (m, 6H), 3.92 (s, 3H), 3.37-3.27 (m, 6H), 3.26-3.09 (m, 2H), 3.12 (br d, J=8.6 Hz, 2H), 2.94-2.84 (m, 1H), 2.80-2.71 (m, 2H), 2.64-2.57 (m, 2H), 2.13-1.87 (m, 7H), 1.58-1.40 (m, 2H), 1.27 (br d, J=6.6 Hz, 6H).

### Example 211. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(7-(2-(2,6-dioxopiperidin-3-yl)-1,3-dioxoisoindolin-4-yl)-2,7-diazaspiro[3.5]nonan-2-yl)piperidin-1-yl)-3-methoxybenzamide (Compound 211)

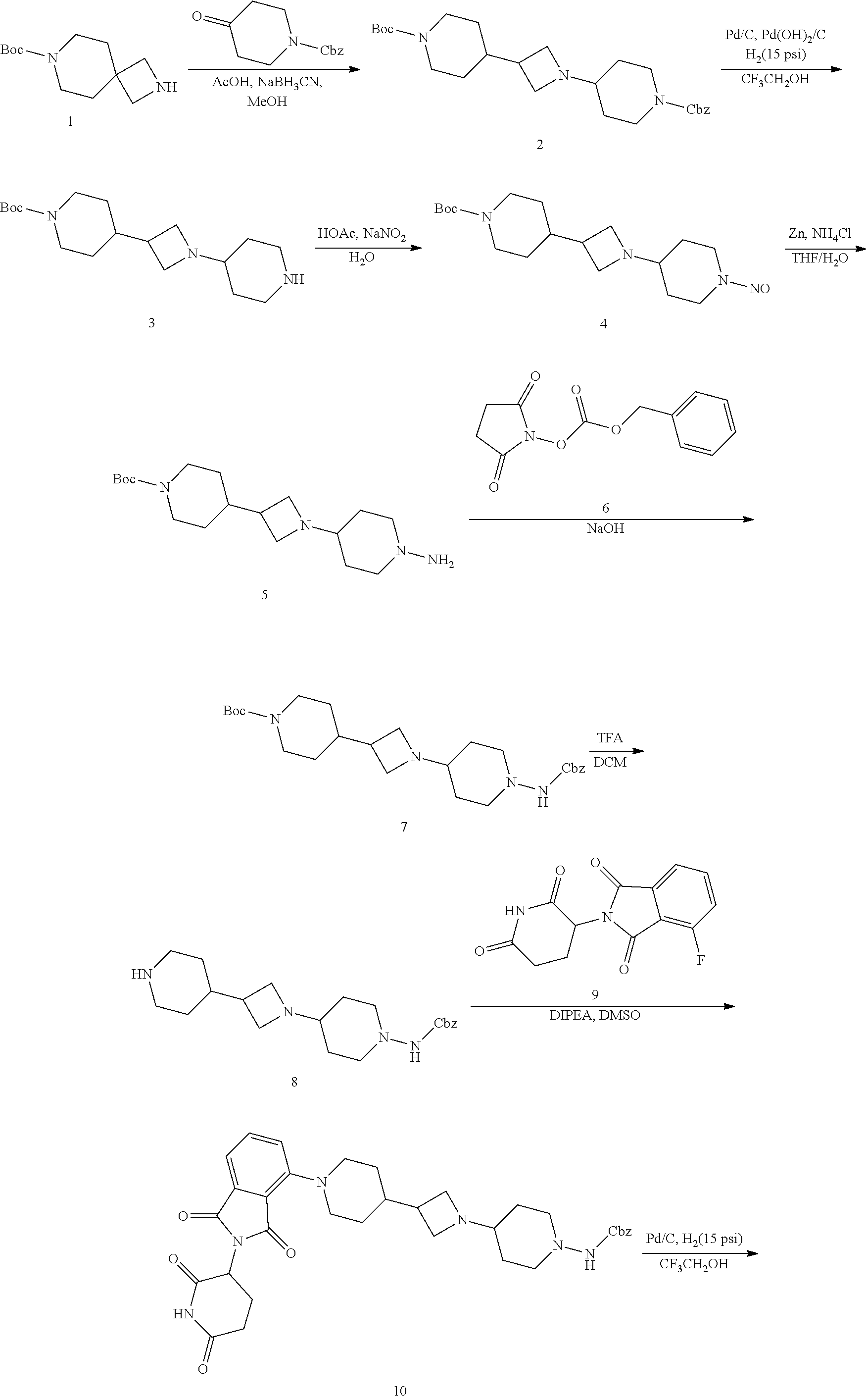

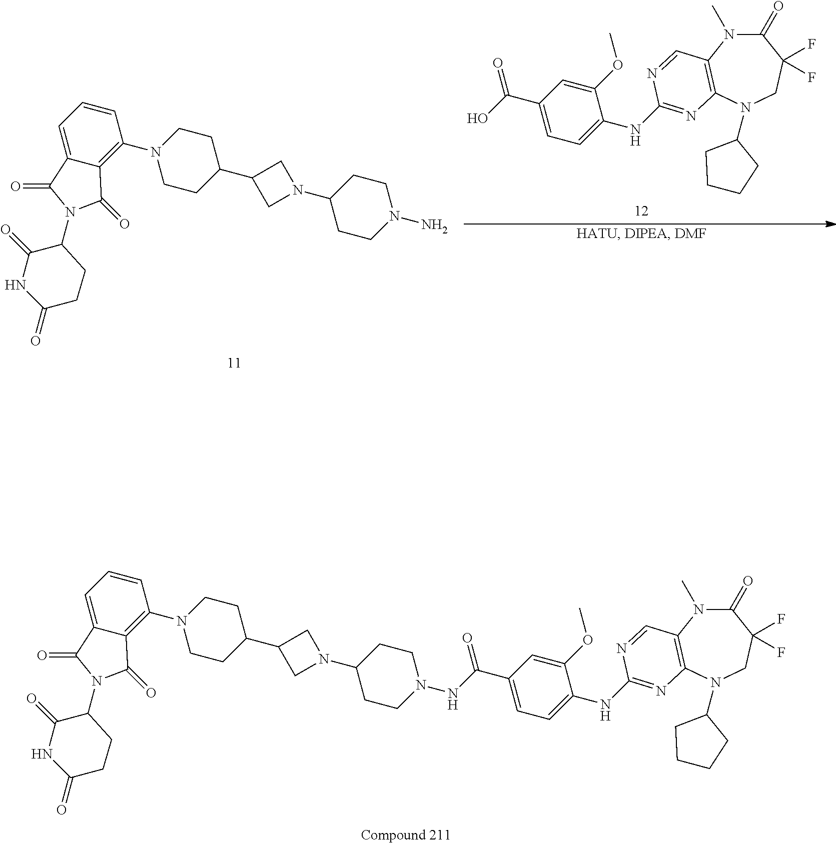 [SMILES: NN1CCC(N2CC(C3CCN(c4cccc5c4C(=O)N(C4CCC(=O)NC4=O)C5=O)CC3)C2)CC1 ; COc1cc(C(=O)O)ccc1Nc1ncc2c(n1)N(C1CCCC1)CC(F)(F)C(=O)N2C ; "HATU, DIPEA, DMF" ; COc1cc(C(=O)NN2CCC(N3CC(C4CCN(c5cccc6c5C(=O)N(C5CCC(=O)NC5=O)C6=O)CC4)C3)CC2)ccc1Nc1ncc2c(n1)N(C1CCCC1)CC(F)(F)C(=O)N2C]

### Step 1. Synthesis of tert-butyl 2-(1-((benzyloxy) carbonyl) piperidin-4-yl)-2,7-diazaspiro[3.5]nonane-7-carboxylate (2)

To a solution of tert-butyl 2,7-diazaspiro[3.5]nonane-7-carboxylate (4 g, 17.67 mmol) and benzyl 4-oxopiperidine-1-carboxylate (4.12 g, 17.67 mmol, 3.52 mL) in MeOH (200 mL) was added AcOH (1.06 g, 17.67 mmol, 1.01 mL) at 0° C. Then NaBH₃CN (3.33 g, 53.02 mmol) was added slowly at 0° C. The mixture was stirred at 0˜20° C. for 16 hr. LCMS showed starting material was consumed completely and a peak (88%) with desired mass. The reaction mixture was diluted with H₂O (300 mL) and extracted with EtAOc (300 mL×3). The combined organic layer was washed with saturated NaHCO₃ (300 mL×3), dried over Na₂SO₄, filtered. The filtrate was concentrated. Compound tert-butyl 2-(1-((benzyloxy) carbonyl) piperidin-4-yl)-2,7-diazaspiro[3.5]nonane-7-carboxylate (7.6 g, crude) was obtained as a yellow oil. MS(M+H)⁺=444.3

### Step 2. Synthesis of tert-butyl 2-(piperidin-4-yl)-2,7-diazaspiro[3.5]nonane-7-carboxylate (3)

To a solution of tert-butyl 2-(1-((benzyloxy) carbonyl) piperidin-4-yl)-2,7-diazaspiro[3.5]nonane-7-carboxylate (15 g, 33.82 mmol) in CF₃CH₂OH (100 mL) was added Pd/C (1.5 g, 10% purity) and Pd(OH)₂/C (1.5 g, 20% purity) under N₂ atmosphere. The suspension was degassed and purged with H₂ for 3 times. The mixture was stirred at 20˜50° C. for 16 hr under H₂ (15 Psi). LCMS showed starting material remained and a peak with desired mass. Additional Pd(OH)₂/C (1.5 g, 20% purity) and Pd/C (1.5 g, 10% purity) were added under N₂ atmosphere. The suspension was degassed and purged with H₂ for 3 times. The mixture was stirred at 60° C. for 16 hr under H₂ (15 Psi). LCMS showed starting material was consumed completely and a peak with desired mass. The reaction mixture was diluted with CF₃CH₂OH (500 mL) and filtered. The filtrate was concentrated in vacuum to afford tert-butyl 2-(piperidin-4-yl)-2,7-diazaspiro[3.5]nonane-7-carboxylate (18 g, crude) as a yellow oil. MS(M+H)⁺=310.54

### Step 3. Synthesis of tert-butyl 2-(1-nitrosopiperidin-4-yl)-2,7-diazaspiro[3.5]nonane-7-carboxylate (4)

To a solution of tert-butyl 2-(piperidin-4-yl)-2,7-diazaspiro[3.5]nonane-7-carboxylate (18 g, 58.17 mmol) in H₂O (200 mL) was added NaNO₂ (12.04 g, 174.51 mmol) at 0° C., then AcOH (13.97 g, 232.67 mmol, 13.31 mL) was added drop-wise at 0° C. The mixture was stirred at 0-20° C. for 2 hr. LCMS showed starting material was consumed completely and a peak (94%) with desired mass. The pH of the reaction mixture was adjusted to around 10 with diluted with saturated NaHCO₃ (200 mL) at 0° C. and extracted with a mixture solvent (EtOAc:MeOH=10:1, 200 mL×3). The combined organic layer was dried over Na₂SO₄, filtered. The filtrate was concentrated in vacuum to afford tert-butyl 2-(1-nitrosopiperidin-4-yl)-2,7-diazaspiro[3.5]nonane-7-carboxylate (7.69 g, 22.72 mmol, 39.06% yield) as a yellow solid. MS(M+H)⁺=339.2

### Step 4. Synthesis of tert-butyl 2-(1-(((benzyloxy)carbonyl)amino)piperidin-4-yl)-2,7-diazaspiro[3.5]nonane-7-carboxylate (7)

To a solution of tert-butyl 2-(1-nitrosopiperidin-4-yl)-2,7-diazaspiro[3.5]nonane-7-carboxylate (7.69 g, 22.72 mmol) in THF (80 mL) and H₂O (80 mL) was added Zn (11.89 g, 181.77 mmol), then NH₄Cl (19.45 g, 363.55 mmol) was added portion wise at 0° C. The mixture was stirred at 0˜20° C. for 12 hr. LCMS showed the starting material was consumed completely and a peak with mass (97%) of tert-butyl 2-(1-aminopiperidin-4-yl)-2,7-diazaspiro[3.5]nonane-7-carboxylate. The reaction mixture was filtered. To the filtrate was added NaOH (5.45 g, 136.33 mmol) and benzyl (2,5-dioxopyrrolidin-1-yl) carbonate (11.33 g, 45.44 mmol) and the resulting mixture was stirred at 20° C. for 3 h. The reaction mixture was extracted with EtOAc (200 mL×3). The organic layer was dried over Na₂SO₄, filtered and concentrated in vacuum to afford the byproduct of tert-butyl 2-(1-((benzyloxy)carbonyl)piperidin-4-yl)-2,7-diazaspiro[3.5]nonane-7-carboxylate. To the aqueous phase was added benzyl (2,5-dioxopyrrolidin-1-yl) carbonate (11.33 g, 45.44 mmol) at 20° C. and the resulting mixture was stirred at 0˜20° C. for 24 hr. LCMS showed tert-butyl 2-(1-aminopiperidin-4-yl)-2,7-diazaspiro[3.5]nonane-7-carboxylate remained and a peak (7%) with desired mass. The reaction mixture was extracted with EtOAc (200 mL×3). The organic layer was dried over Na₂SO₄, filtered and concentrated in vacuum. The residue was purified by flash silica gel chromatography (25 g SepaFlash® Silica Flash Column, Eluent of 0˜100% EtOAc/Petroleum ether to 0˜10% of Dichloromethane/Methanol gradient @100 mL/min) to afford the title compound (1.1 g, 2.40 mmol) as a white solid. To the aqueous phase was added benzyl (2,5-dioxopyrrolidin-1-yl) carbonate (11.33 g, 45.44 mmol) at 20° C. and the mixture was stirred at 0˜20° C. for 12 hr. LCMS showed tert-butyl 2-(1-aminopiperidin-4-yl)-2,7-diazaspiro[3.5]nonane-7-carboxylate remained and a peak (9%) with desired mass. The reaction mixture was extracted with EtOAc (200 mL×3). The organic layer was dried over Na₂SO₄, filtered and concentrated in vacuum. The residue was purified by flash silica gel chromatography (25 g SepaFlash® Silica Flash Column, Eluent of 0˜100% EtOAc/Petroleum ether to 0˜10% of Dichloromethane/Methanol gradient @100 mL/min) to afford the title compound (1.9 g, 2.40 mmol) as a white solid. To the aqueous phase was added benzyl (2,5-dioxopyrrolidin-1-yl) carbonate (11.33 g, 45.44 mmol) at 20° C. and the mixture was stirred at 0˜20° C. for 12 hr. LCMS showed tert-butyl 2-(1-aminopiperidin-4-yl)-2,7-diazaspiro[3.5]nonane-7-carboxylate remained and a peak (6%) with desired mass. The reaction mixture was extracted with EtOAc (200 mL×3). The organic layer was dried over Na₂SO₄, filtered and concentrated in vacuum. The residue was purified by flash silica gel chromatography (25 g SepaFlash® Silica Flash Column, Eluent of 0˜100% EtOAc/Petroleum ether to 0˜10% of Dichloromethane/Methanol gradient @100 mL/min) to afford the title compound (1 g, 2.40 mmol) as a white solid. To the aqueous phase was added benzyl (2,5-dioxopyrrolidin-1-yl) carbonate (11.33 g, 45.44 mmol) at 20° C. and the mixture was stirred at 0˜20° C. for 12 hr. LCMS showed a peak (1%) with desired mass. The reaction mixture was extracted with EtOAc (200 mL×3). The organic layer was dried over Na₂SO₄, filtered and concentrated in vacuum. The residue was purified by flash silica gel chromatography (25 g SepaFlash® Silica Flash Column, Eluent of 0˜100% EtOAc/Petroleum ether to 0˜10% of Dichloromethane/Methanol gradient @100 mL/min) to afford the title compound (337 mg) as a white solid. MS(M+H)⁺=459.26

### Step 5. Synthesis of benzyl (4-(2,7-diazaspiro[3.5]nonan-2-yl)piperidin-1-yl)carbamate (8)

To a solution of tert-butyl 2-(1-(((benzyloxy)carbonyl)amino)piperidin-4-yl)-2,7-diazaspiro[3.5]nonane-7-carboxylate (1.9 g, 4.14 mmol) in DCM (8 mL) was added TFA (5.67 g, 49.72 mmol, 3.68 mL) at 20° C. The mixture was stirred at 20° C. for 4 hr. LCMS showed starting material was consumed completely and a peak (56%) with desired mass. The reaction mixture was concentrated in vacuum at 20° C. to afford benzyl (4-(2,7-diazaspiro[3.5]nonan-2-yl)piperidin-1-yl)carbamate (3.1 g, crude, TFA salt) as a yellow oil. MS(M+H)⁺=359.27

### Step 6. Synthesis of benzyl (4-(7-(2-(2,6-dioxopiperidin-3-yl)-1,3-dioxoisoindolin-4-yl)-2,7-diazaspiro[3.5]nonan-2-yl)piperidin-1-yl)carbamate (10)

To a solution of benzyl (4-(2,7-diazaspiro[3.5]nonan-2-yl)piperidin-1-yl)carbamate (3.08 g, 6.52 mmol, TFA salt) in DMSO (10 mL) was added DIPEA (4.21 g, 32.58 mmol, 5.68 mL) and 2-(2,6-dioxopiperidin-3-yl)-4-fluoroisoindoline-1,3-dione (900 mg, 3.26 mmol) at 20° C. The mixture was stirred at 20˜100° C. for 16 hr. LCMS showed the starting material 1 was consumed completely and a peak (44%) with desired mass. The reaction mixture was diluted with H₂O (30 mL) and extracted with EtOAc (30 mL×3). The combined organic layer was washed with brine (30 mL×3), dried over Na₂SO₄, filtered and concentrated. The residue was purified by flash silica gel chromatography (25 g SepaFlash® Silica Flash Column, Eluent of 0˜100% EtOAc/Petroleum ether to 0˜10% Dichloromethane/Methanol gradient @100 mL/min). Compound benzyl (4-(7-(2-(2,6-dioxopiperidin-3-yl)-1,3-dioxoisoindolin-4-yl)-2,7-diazaspiro[3.5]nonan-2-yl)piperidin-1-yl)carbamate (1.27 g, 2.07 mmol, 63.41% yield, 100% purity) was obtained as a yellow solid. MS(M+H)⁺=615.2

### Step 8. Synthesis of 4-(2-(1-aminopiperidin-4-yl)-2,7-diazaspiro[3.5]nonan-7-yl)-2-(2,6-dioxopiperidin-3-yl) isoindoline-1,3-dione (11)

To a solution of benzyl (4-(7-(2-(2,6-dioxopiperidin-3-yl)-1,3-dioxoisoindolin-4-yl)-2,7-diazaspiro[3.5]nonan-2-yl)piperidin-1-yl)carbamate (1.17 g, 1.90 mmol) in CF₃CH₂OH (20 mL) was added Pd/C (0.6 g, 10% purity) under N₂ atmosphere. The suspension was degassed and purged with H₂ for 3 times. The mixture was stirred at 15° C. for 16 hr under H₂ (15 psi). LCMS showed the starting material was consumed completely and a peak (73%) with desired mass. The reaction mixture was diluted with CF₃CH₂OH (90 mL) and filtered. The filtrate was concentrated in vacuum to afford 4-(2-(1-aminopiperidin-4-yl)-2,7-diazaspiro[3.5]nonan-7-yl)-2-(2,6-dioxopiperidin-3-yl) isoindoline-1,3-dione (865 mg, crude) as a green solid. MS(M+H)⁺=481.39

### Step 9. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(7-(2-(2,6-dioxopiperidin-3-yl)-1,3-dioxoisoindolin-4-yl)-2,7-diazaspiro[3.5]nonan-2-yl)piperidin-1-yl)-3-methoxybenzamide (Compound 211)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (500 mg, 1.12 mmol) in DMF (4 mL) was added HATU (467.39 mg, 1.23 mmol) and DIPEA (433.28 mg, 3.35 mmol, 583.94 μL). The mixture was stirred at 20° C. for 10 min, then a solution of 4-(2-(1-aminopiperidin-4-yl)-2,7-diazaspiro[3.5]nonan-7-yl)-2-(2,6-dioxopiperidin-3-yl) isoindoline-1,3-dione (644.42 mg, 1.34 mmol) in DMF (4 mL) was added. The mixture was stirred for 1 h. LCMS showed starting material was consumed completely and a peak (49%) with desired mass. The reaction mixture was diluted with H₂O (30 mL) and extracted with EtOAc (30 mL×3). The organic layer was washed with brine (30 mL×3), dried over Na₂SO₄, filtered and concentrated. The residue was purified by prep-HPLC (column: Phenomenex luna C18 150*40 mm*15 um; mobile phase: [water(FA)-ACN]; B %: 15%-45%, 10 min) to afford product A (462 mg) and product B (242 mg). The product A was re-purified by prep-HPLC (column: Waters Xbridge C18 150*50 mm*10 um; mobile phase: [water(NH₄HCO₃)-ACN]; B %: 39%-69%, 10 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(7-(2-(2,6-dioxopiperidin-3-yl)-1,3-dioxoisoindolin-4-yl)-2,7-diazaspiro[3.5]nonan-2-yl)piperidin-1-yl)-3-methoxybenzamide (155.9 mg, 162.76 μmol, 14.56% yield, 95% purity) as a yellow solid. The product B was re-purified by prep-HPLC (column: Waters Xbridge C18 150*50 mm*10 um; mobile phase: [water(NH₄HCO₃)-ACN]; B %: 39%-69%, 10 min) followed by prep-HPLC (column: Phenomenex Synergi Polar-RP 100*25 mm*4 um; mobile phase: [water (TFA)-ACN]; B %: 32%-52%, 7 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(7-(2-(2,6-dioxopiperidin-3-yl)-1,3-dioxoisoindolin-4-yl)-2,7-diazaspiro[3.5]nonan-2-yl)piperidin-1-yl)-3-methoxybenzamide (110 mg, 120.88 μmol, 10.82% yield) as a yellow solid. MS(M+H)⁺=910.2

¹H NMR (400 MHz, DMSO-d₆) δ=11.09 (br s, 1H), 9.33 (s, 1H), 8.32-8.20 (m, 2H), 7.95 (s, 1H), 7.67 (br t, J=7.8 Hz, 1H), 7.47-7.39 (m, 2H), 7.33 (br d, J=7.6 Hz, 2H), 5.10 (br dd, J=5.4, 12.7 Hz, 1H), 4.76 (br t, J=7.8 Hz, 1H), 4.04 (br t, J=14.0 Hz, 2H), 3.93 (s, 3H), 3.33 (br s, 3H), 3.21 (br s, 4H), 2.98 (br s, 4H), 2.87-2.75 (m, 3H), 2.71-2.50 (m, 1H), 2.56 (br d, J=9.3 Hz, 2H), 2.10 (br s, 1H), 2.06-2.00 (m, 1H), 1.94 (br s, 3H), 1.83 (br s, 4H), 1.70 (br d, J=10.4 Hz, 4H), 1.59 (br s, 4H), 1.31 (br d, J=9.3 Hz, 2H).

### Example 212. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(7-(2-(2,6-dioxopiperidin-3-yl)-1,3-dioxoisoindolin-5-yl)-2,7-diazaspiro[3.5]nonan-2-yl)piperidin-1-yl)-3-methoxybenzamide (Compound 212)

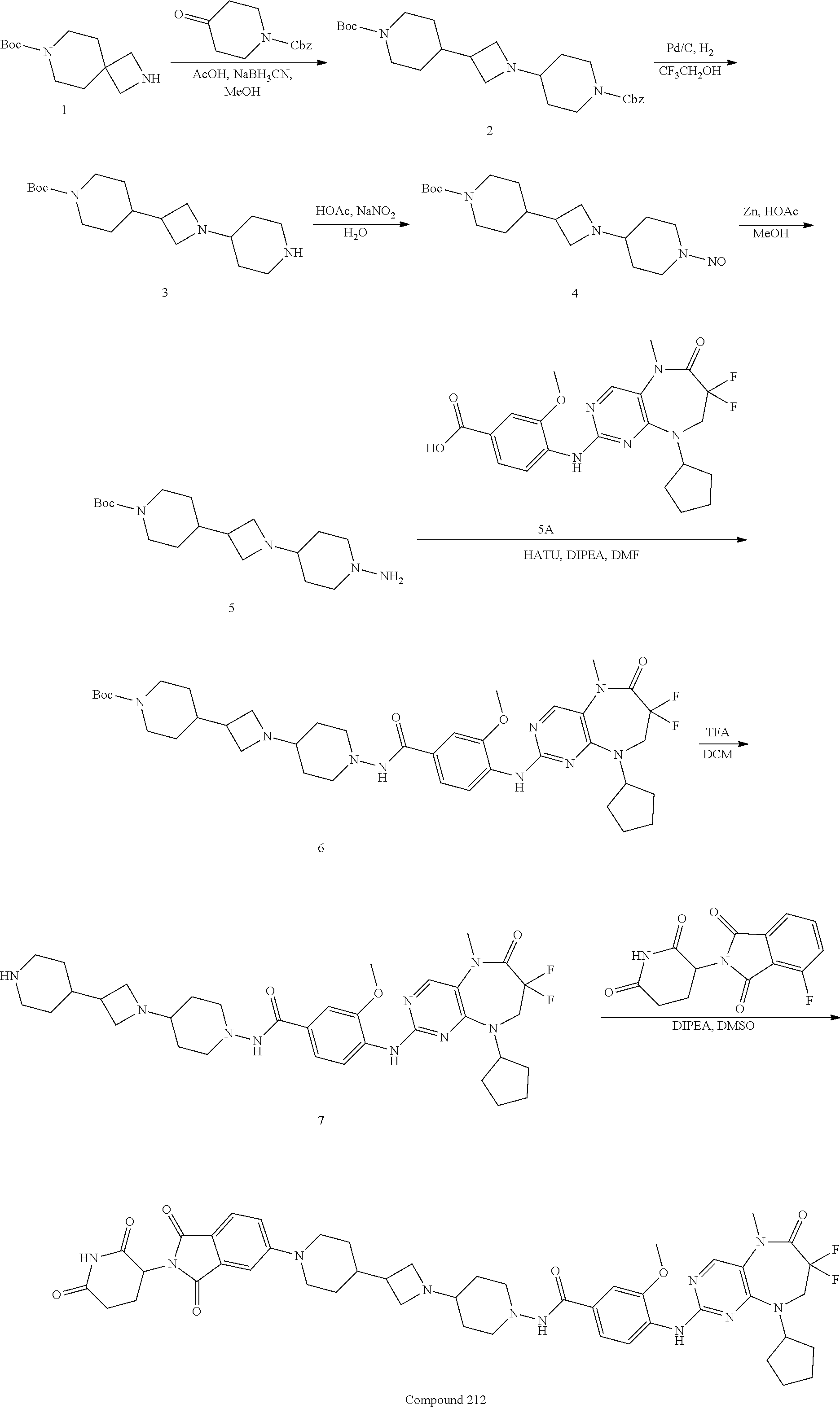

### Step 1. Synthesis of tert-butyl 2-(1-((benzyloxy)carbonyl)piperidin-4-yl)-2,7-diazaspiro[3.5]nonane-7-carboxylate (2)

To a solution of tert-butyl 2,7-diazaspiro[3.5]nonane-7-carboxylate (2 g, 8.84 mmol) and benzyl 4-oxopiperidine-1-carboxylate (2.06 g, 8.84 mmol, 1.76 mL) in MeOH (50 mL) was added AcOH (530.70 mg, 8.84 mmol, 505.42 μL) at 0° C., then NaBH₃CN (1.67 g, 26.51 mmol) was added slowly at 0° C. and the resulting mixture was stirred at 20° C. for 16 h. LCMS showed starting material was consumed completely and a major peak (99%) with desired mass. The reaction mixture was diluted with H₂O (100 mL) and extracted with EtOAc (100 mL×3). The organic layer was washed with saturated NaHCO₃ (100 mL×3), dried over Na₂SO₄, filtered and concentrated to afford tert-butyl 2-(1-((benzyloxy)carbonyl)piperidin-4-yl)-2,7-diazaspiro[3.5]nonane-7-carboxylate (3.92 g, crude) as a yellow oil. MS(M+H)⁺=444.4

### Step 2. Synthesis of tert-butyl 2-(piperidin-4-yl)-2,7-diazaspiro[3.5]nonane-7-carboxylate (3)

To a solution of tert-butyl 2-(1-((benzyloxy)carbonyl)piperidin-4-yl)-2,7-diazaspiro[3.5]nonane-7-carboxylate (3.92 g, 8.84 mmol) in CF₃CH₂OH (50 mL) was added Pd/C (1 g, 10% purity) under N₂ atmosphere. The suspension was degassed and purged with H₂ for 3 times. The mixture was stirred at 20° C. for 16 h under H₂ (15 Psi). LCMS showed 79% of starting material remained, additional Pd/C (1 g, 10% purity) was added under N2 and the resulting mixture was stirred at 20° C. for 16 h under H₂ (15 Psi). LCMS showed starting material was consumed completely and a peak with desired mass. The reaction mixture was diluted with EtOH (150 mL) and filtered. The filtrate was concentrated in vacuum to afford tert-butyl 2-(piperidin-4-yl)-2,7-diazaspiro[3.5]nonane-7-carboxylate (4 g, crude) as a colorless oil. MS(M+H)⁺=310.3

### Step 3. Synthesis of tert-butyl 2-(1-nitrosopiperidin-4-yl)-2,7-diazaspiro[3.5]nonane-7-carboxylate (4)

To a solution of tert-butyl 2-(piperidin-4-yl)-2,7-diazaspiro[3.5]nonane-7-carboxylate (4 g, 12.93 mmol) in H₂O (50 mL) was added NaNO₂ (1.78 g, 25.85 mmol) at 0° C., then AcOH (2.33 g, 38.78 mmol, 2.22 mL) was added drop-wise at 0° C. and the resulting mixture was stirred at 20° C. for 4 h. LCMS showed starting material was consumed completely and a major peak (98%) with desired mass. Added saturated NaHCO₃ (150 mL) to this reaction mixture to adjust the pH=9 and extracted with EtOAc (200 mL×5). The organic layer was dried over Na₂SO₄, filtered and concentrated to afford tert-butyl 2-(1-nitrosopiperidin-4-yl)-2,7-diazaspiro[3.5]nonane-7-carboxylate (3 g, 8.86 mmol, 68.57% yield) as a yellow oil. MS(M+H)⁺=339.4

### Step 4. Synthesis of tert-butyl 2-(1-aminopiperidin-4-yl)-2,7-diazaspiro[3.5]nonane-7-carboxylate (5)

To a solution of tert-butyl 2-(1-nitrosopiperidin-4-yl)-2,7-diazaspiro[3.5]nonane-7-carboxylate (3 g, 8.86 mmol) in MeOH (50 mL) was added Zn (3.01 g, 46.09 mmol) slowly at 0° C., then AcOH (7.98 g, 132.96 mmol, 7.60 mL) was added drop-wise at 0° C. and the resulting mixture was stirred at 20° C. for 14 h. LCMS showed starting material was consumed completely and a peak with desired mass. The reaction mixture was filtered and the filtrate was concentrated in vacuum to afford tert-butyl 2-(1-aminopiperidin-4-yl)-2,7-diazaspiro[3.5]nonane-7-carboxylate (9 g, crude) as a yellow oil. MS(M+H)⁺=325.5

### Step 5. Synthesis of tert-butyl 2-(1-(4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamido)piperidin-4-yl)-2,7-diazaspiro[3.5]nonane-7-carboxylate (6)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzoic acid (400 mg, 893.99 μmol) in DMF (5 mL) were added HATU (373.91 mg, 983.38 μmol) and DIPEA (231.08 mg, 1.79 mmol, 311.43 μL), the mixture was stirred at 20° C. for 10 min, then a solution of tert-butyl 2-(1-aminopiperidin-4-yl)-2,7-diazaspiro[3.5]nonane-7-carboxylate (870.19 mg, 2.68 mmol) with DIPEA (346.62 mg, 2.68 mmol, 467.15 μL) in DMF (5 mL) was added and the resulting mixture was stirred at 20° C. for 1 h. LCMS showed all starting material was consumed completely and a peak (82%) with desired mass. The reaction mixture was diluted with H₂O (30 mL) and extracted with EtOAc (30 mL×3). The organic layer was washed with brine (30 mL×3), dried over Na₂SO₄, filtered and concentrated. The residue was purified by flash silica gel chromatography (12 g SepaFlash® Silica Flash Column, Eluent of 0˜100% EtOAc/Petroleum ether gradient @100 mL/min) to afford tert-butyl 2-[1-[[4-[(9-cyclopentyl-7,7-difluoro-5-methyl-6-oxo-8H-pyrimido[4,5-b][1,4]diazepin-2-yl)amino]-3-methoxy-benzoyl]amino]-4-piperidyl]-2,7-diazaspiro[3.5]nonane-7-carboxylate (302 mg, 400.59 μmol, 44.81% yield) as an off-white solid. MS(M+H)⁺=754.2

### Step 6. Synthesis of N-(4-(2,7-diazaspiro[3.5]nonan-2-yl)piperidin-1-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (7)

To a solution of tert-butyl 2-[1-[[4-[(9-cyclopentyl-7,7-difluoro-5-methyl-6-oxo-8H-pyrimido[4,5-b][1,4]diazepin-2-yl)amino]-3-methoxy-benzoyl]amino]-4-piperidyl]-2,7-diazaspiro[3.5]nonane-7-carboxylate (300 mg, 397.94 μmol) in DCM (5 mL) was added TFA (272.25 mg, 2.39 mmol, 176.78 μL) at 20° C. and the resulting mixture was stirred at 20° C. for 1 h. LCMS showed starting material was consumed completely and a peak (43%) with desired mass. The reaction mixture was concentrated in vacuum to afford N-(4-(2,7-diazaspiro[3.5]nonan-2-yl)piperidin-1-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (763 mg, crude, TFA) as a yellow oil. MS(M+H)⁺=654.3

### Step 7. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(4-(7-(2-(2,6-dioxopiperidin-3-yl)-1,3-dioxoisoindolin-5-yl)-2,7-diazaspiro[3.5]nonan-2-yl)piperidin-1-yl)-3-methoxybenzamide (Compound 212)

To a solution of 2-(2,6-dioxo-3-piperidyl)-5-fluoro-isoindoline-1,3-dione (30 mg, 108.61 μmol) in DMSO (3 mL) were added DIPEA (140.37 mg, 1.09 mmol, 189.18 μL) and N-(4-(2,7-diazaspiro[3.5]nonan-2-yl)piperidin-1-yl)-4-((9-cyclopentyl-7,7-difluoro-5-methyl-6-oxo-6,7,8,9-tetrahydro-5H-pyrimido[4,5-b][1,4]diazepin-2-yl)amino)-3-methoxybenzamide (213.32 mg, 119.47 μmol, 43% purity, TFA) at 20° C. and the resulting mixture was stirred at 80° C. for 16 h. LCMS showed starting material was consumed completely and a peak (33%) with desired mass. The reaction mixture was diluted with H₂O (12 mL) and extracted with EtAOc (12 mL×3). The organic layer was washed with brine (12 mL×3), dried over Na₂SO₄, filtered and concentrated. The residue was purified by prep-TLC (SiO₂, Dichloromethane:Methanol=10:1) followed by prep-HPLC (column: Waters Xbridge 150*25 mm*5 μm; mobile phase: [water(NH₄HCO₃)-ACN]; B %: 37%-67%, 8 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(4-(7-(2-(2,6-dioxopiperidin-3-yl)-1,3-dioxoisoindolin-5-yl)-2,7-diazaspiro[3.5]nonan-2-yl)piperidin-1-yl)-3-methoxybenzamide (9.3 mg, 9.91 μmol, 9.13% yield, 97% purity) as a yellow solid. MS(M+H)⁺=910.1

¹H NMR (400 MHz, CD₃CN) δ=9.38-8.60 (m, 1H), 8.46 (d, J=8.2 Hz, 1H), 8.10 (s, 1H), 7.82 (s, 1H), 7.71 (s, 1H), 7.65-7.58 (m, 1H), 7.42-7.32 (m, 2H), 7.28 (d, J=2.2 Hz, 1H), 7.15 (dd, J=2.3, 8.6 Hz, 1H), 4.96-4.90 (m, 1H), 4.89-4.81 (m, 1H), 3.99-3.91 (m, 5H), 3.44-3.35 (m, 4H), 3.32 (s, 3H), 3.10-3.03 (m, 2H), 3.02 (s, 3H), 2.79-2.60 (m, 6H), 2.09-1.98 (m, 4H), 1.82-1.77 (m, 4H), 1.77-1.70 (m, 4H), 1.69-1.59 (m, 4H), 1.46-1.25 (m, 2H).

### Example 213. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(2-(2,6-dioxopiperidin-3-yl)-1,3-dioxoisoindolin-4-yl)-2,7-diazaspiro[3.5]nonan-2-yl)piperidin-1-yl)-3-methoxybenzamide (Compound 213)

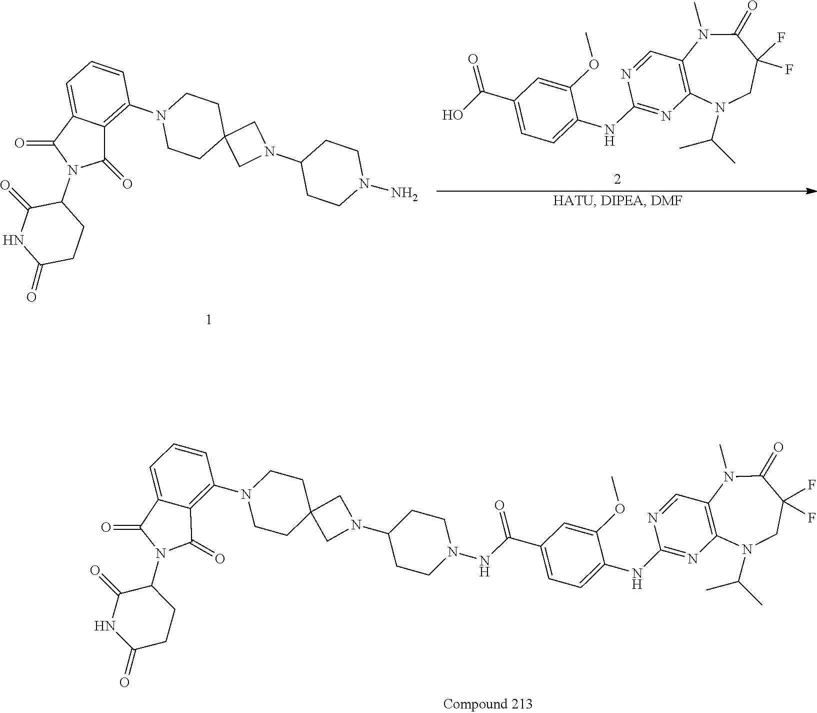 [SMILES: NN1CCC(N2CC3(CCN(c4cccc5c4C(=O)N(C4CCC(=O)NC4=O)C5=O)CC3)C2)CC1 ; COc1cc(C(=O)O)ccc1Nc1ncc2c(n1)N(C(C)C)CC(F)(F)C(=O)N2C ; "HATU, DIPEA, DMF" ; COc1cc(C(=O)NN2CCC(N3CC4(CCN(c5cccc6c5C(=O)N(C5CCC(=O)NC5=O)C6=O)CC4)C3)CC2)ccc1Nc1ncc2c(n1)N(C(C)C)CC(F)(F)C(=O)N2C]

The compound 213 was synthesized by the method described in the scheme similar to the method described in Example 211.

MS(M+H)⁺=884.3, ¹H NMR (400 MHz, DMSO-d₆) δ=11.08 (br s, 1H), 9.28 (s, 1H), 8.34-8.27 (m, 1H), 8.22 (s, 1H), 7.87 (s, 1H), 7.68 (dd, J=7.3, 8.3 Hz, 1H), 7.47-7.41 (m, 2H), 7.36-7.30 (m, 2H), 5.10 (dd, J=5.4, 12.9 Hz, 1H), 4.87 (td, J=6.5, 13.3 Hz, 1H), 4.03 (br t, J=13.4 Hz, 2H), 3.93 (s, 3H), 3.32 (s, 3H), 3.27-3.13 (m, 4H), 2.97 (br s, 5H), 2.86 (dd, J=5.4, 17.1 Hz, 1H), 2.82-2.73 (m, 2H), 2.62-2.55 (m, 3H), 2.14-2.07 (m, 1H), 2.05-2.00 (m, 1H), 1.84 (br s, 4H), 1.73-1.63 (m, 2H), 1.37-1.28 (m, 2H), 1.24 (dd, J=0.6 Hz 6H).

### Example 214. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(2-(2,6-dioxopiperidin-3-yl)-1,3-dioxoisoindolin-5-yl)-2,7-diazaspiro[3.5]nonan-2-yl)piperidin-1-yl)-3-methoxybenzamide

### (Compound 214)

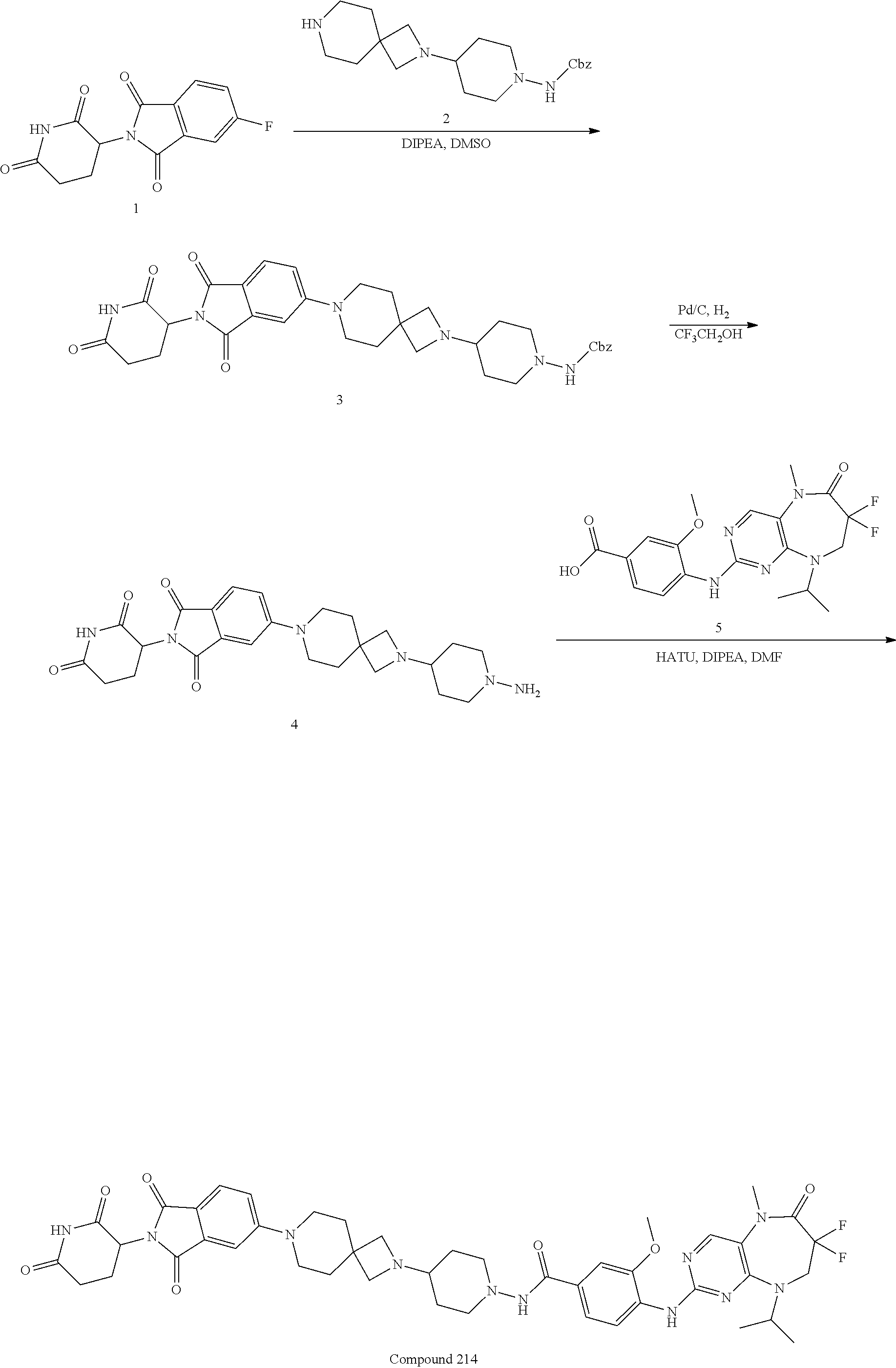

The compound 214 was synthesized by the method described in the scheme similar to the method described in Example 211.

MS(M+H)⁺=884.2, ¹H NMR (400 MHz, DMSO-d₆) δ=11.08 (s, 1H), 10.11-9.91 (m, 1H), 9.55-9.46 (m, 1H), 8.25-8.22 (m, 2H), 7.73-7.65 (m, 1H), 7.49-7.35 (m, 3H), 7.29 (br d, J=8.7 Hz, 1H), 6.96-6.78 (m, 1H), 5.07 (td, J=4.4, 12.7 Hz, 1H), 4.88 (td, J=6.4, 13.3 Hz, 1H), 4.13-3.97 (m, 6H), 3.93 (s, 3H), 3.76-3.60 (m, 1H), 3.58-3.51 (m, 2H), 3.48-3.39 (m, 2H), 3.32 (s, 3H), 3.10 (br d, J=9.0 Hz, 2H), 2.94-2.83 (m, 3H), 2.61 (br d, J=1.6 Hz, 2H), 2.21-2.06 (m, 2H), 2.06-1.94 (m, 3H), 2.01-1.91 (m, 1H), 1.90-1.68 (m, 1H), 1.57-1.41 (m, 2H), 1.24 (d, J=6.7 Hz, 6H).

### Example 215. Synthesis of 5-(2-(1-(4-((7,7-difluoro-9-isopropyl-5-methyl-6-oxo-6,7,8,9-tetrahydro-5H-pyrimido[4,5-b][1,4]diazepin-2-yl)amino)-3-methoxybenzoyl)piperidin-4-yl)-2,7-diazaspiro[3.5]nonan-7-yl)-2-(2,6-dioxopiperidin-3-yl)-6-fluoroisoindoline-1,3-dione (Compound 215)

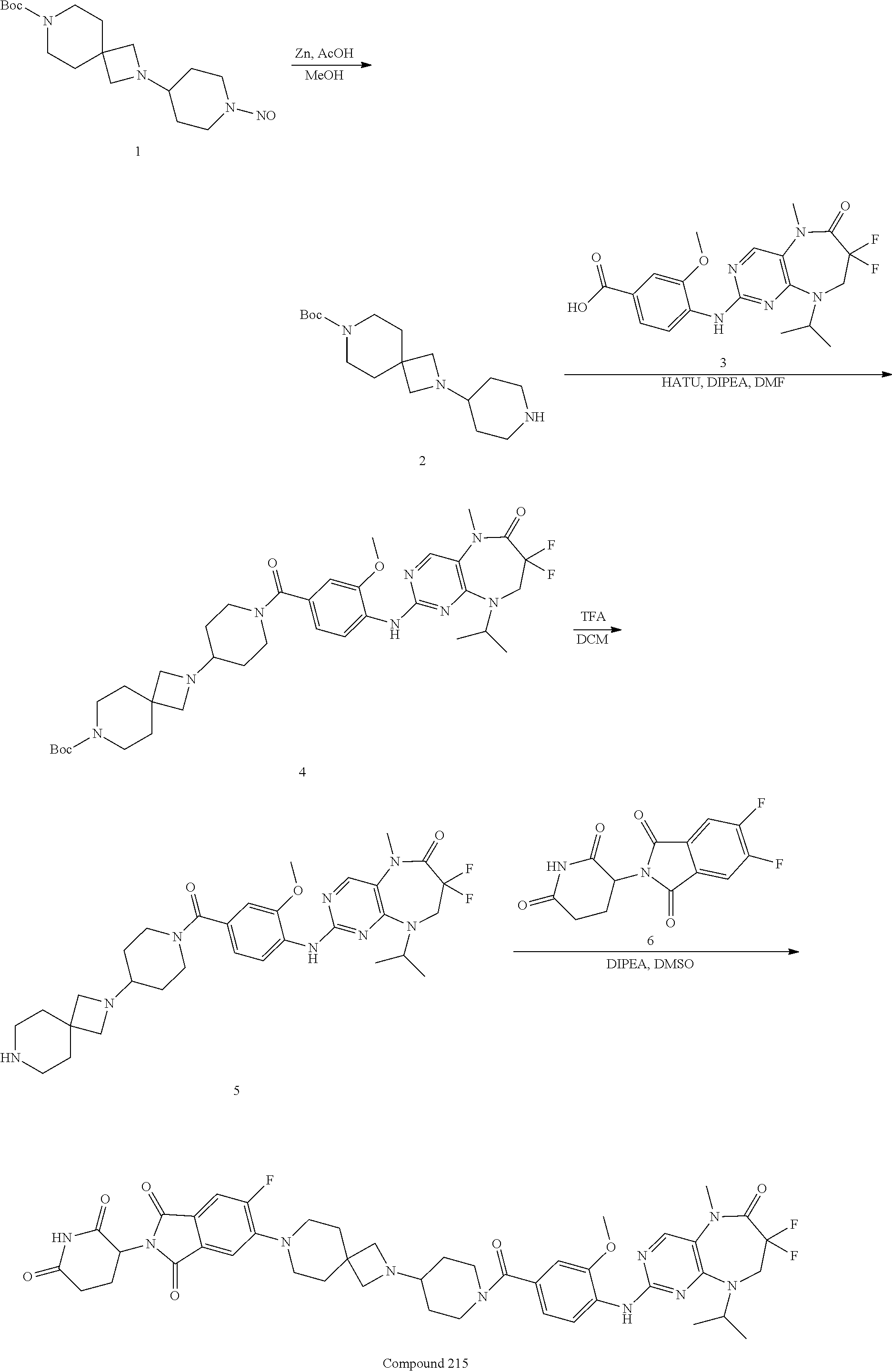

The compound 215 was synthesized by the method described in the scheme similar to the method described in Example 212.

MS(M+H)⁺=887.2, ¹H NMR (400 MHz, DMSO-d₆) δ=11.13-11.06 (m, 1H), 8.25-8.17 (m, 2H), 7.87 (s, 1H), 7.74-7.54 (m, 1H), 7.44 (d, J=7.5 Hz, 1H), 7.06-6.94 (m, 2H), 5.14-5.03 (m, 1H), 4.83 (td, J=6.7, 13.4 Hz, 1H), 4.01 (br t, J=13.6 Hz, 2H), 3.89 (s, 3H), 3.65-3.57 (m, 1H), 3.32 (s, 3H), 3.26-3.14 (m, 4H), 3.04 (br s, 4H), 2.93-2.83 (m, 1H), 2.65-2.59 (m, 1H), 2.58-2.52 (m, 2H), 2.41 (br d, J=8.4 Hz, 1H), 2.10-1.88 (m, 2H), 1.87-1.49 (m, 6H), 1.48-1.29 (m, 1H), 1.21 (d, J=6.7 Hz, 8H).

### Example 216. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(2-(2,6-dioxopiperidin-3-yl)-6-fluoro-1,3-dioxoisoindolin-5-yl)-2,7-diazaspiro[3.5]nonan-2-yl)piperidin-1-yl)-3-methoxybenzamide (Compound 216)

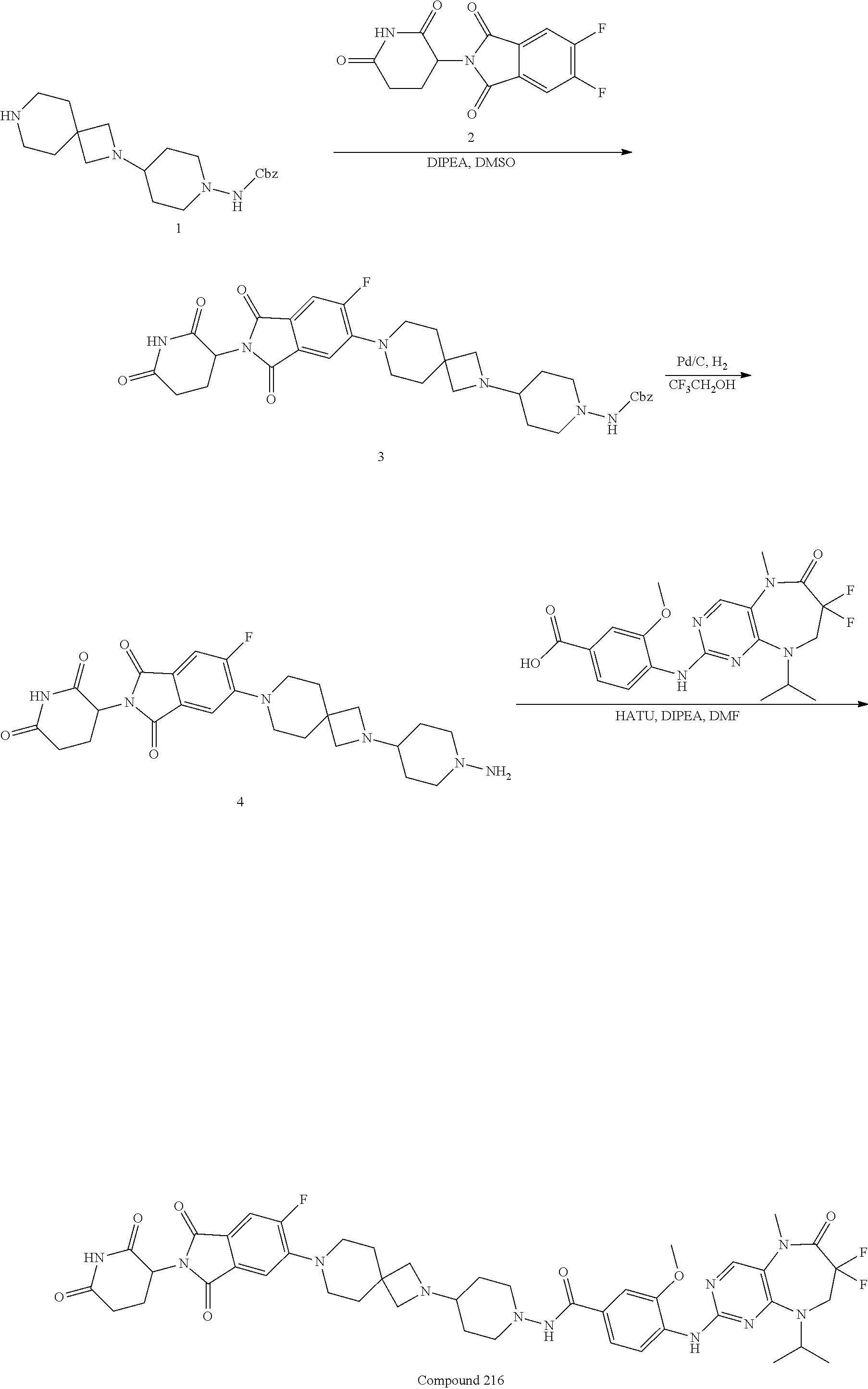

The compound 216 was synthesized by the method described in the scheme similar to the method described in Example 212.

MS(M+H)⁺=902.3, ¹H NMR (400 MHz, DMSO-d₆) δ=11.12 (br s, 1H), 9.30 (br s, 1H), 8.30 (br d, J=8.1 Hz, 1H), 8.22 (s, 1H), 7.87 (s, 1H), 7.71 (br d, J=11.5 Hz, 1H), 7.50-7.38 (m, 3H), 5.10 (br dd, J=5.1, 12.7 Hz, 1H), 4.93-4.82 (m, 1H), 4.04 (br t, J=13.5 Hz, 2H), 3.93 (s, 3H), 3.32 (br s, 3H), 3.18 (br s, 4H), 2.96 (br s, 4H), 2.94-2.82 (m, 2H), 2.77 (br s, 2H), 2.64-2.54 (m, 3H), 2.14-1.99 (m, 2H), 1.81 (br s, 4H), 1.67 (br d, J=2.3 Hz, 2H), 1.38-1.27 (m, 2H), 1.24 (br d, J=6.5 Hz, 6H).

### Example 217. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(2-(2,6-dioxopiperidin-3-yl)-6-fluoro-1,3-dioxoisoindolin-5-yl)-2,7-diazaspiro[3.5]nonan-2-yl)piperidin-1-yl)-2-fluoro-5-methoxybenzamide (Compound 217)

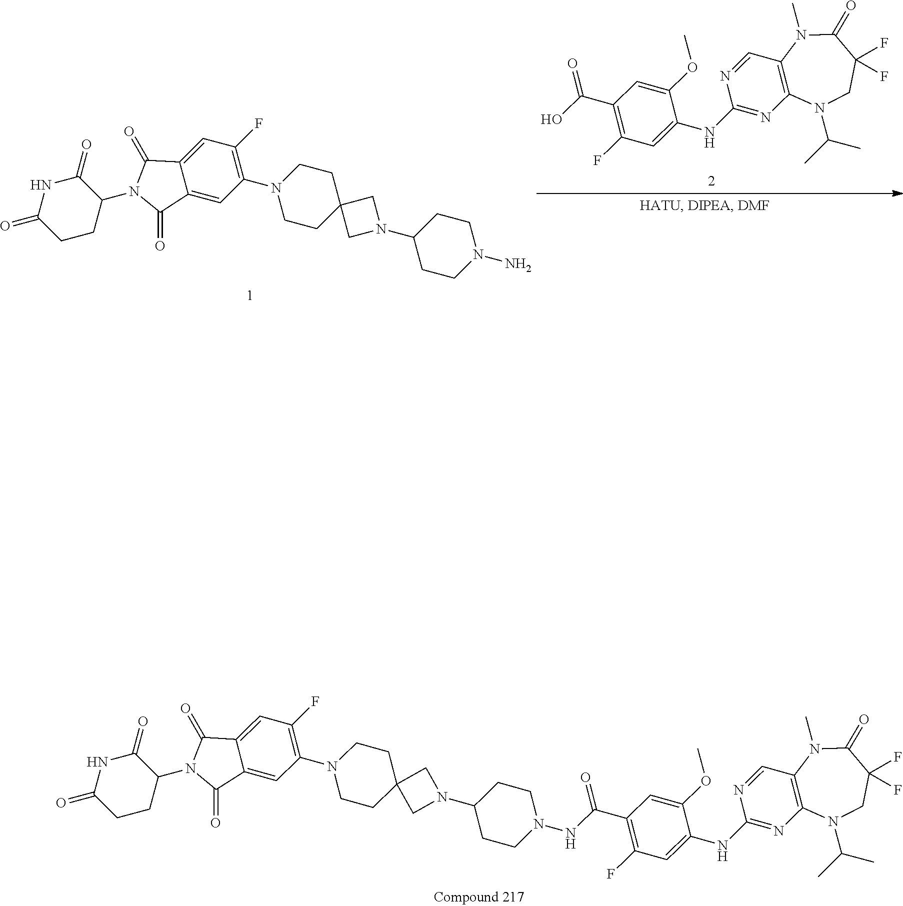 [SMILES: NN1CCC(N2CC3(CCN(c4cc5c(cc4F)C(=O)N(C4CCC(=O)NC4=O)C5=O)CC3)C2)CC1 ; COc1cc(C(=O)O)c(F)cc1Nc1ncc2c(n1)N(C(C)C)CC(F)(F)C(=O)N2C ; COc1cc(C(=O)NN2CCC(N3CC4(CCN(c5cc6c(cc5F)C(=O)N(C5CCC(=O)NC5=O)C6=O)CC4)C3)CC2)c(F)cc1Nc1ncc2c(n1)N(C(C)C)CC(F)(F)C(=O)N2C]

To a solution of 4-((7,7-difluoro-9-isopropyl-5-methyl-6-oxo-6,7,8,9-tetrahydro-5H-pyrimido[4,5-b][1, 4]diazepin-2-yl)amino)-2-fluoro-5-methoxybenzoic acid (80 mg, 182.07 μmol) in DMF (2 mL) were added HATU (76.15 mg, 200.28 μmol) and DIPEA (70.59 mg, 546.21 μmol, 95.14 μL). The mixture was stirred at 20° C. for 10 min, then a solution of 5-(2-(1-aminopiperidin-4-yl)-2,7-diazaspiro[3.5]nonan-7-yl)-2-(2,6-dioxopiperidin-3-yl)-6-fluoroisoindoline-1,3-dione (108.93 mg, 218.49 μmol) in DMF (2 mL) was added and the resulting mixture was stirred at 20° C. for 1 h. LCMS showed all starting material was consumed completely and a peak (48%) with desired mass. The reaction mixture was diluted with H₂O (12 mL) and extracted with EtOAc (12 mL×3). The organic layer was washed with brine (12 mL×3), dried over Na₂SO₄, filtered and concentrated. The residue was purified by prep-HPLC (column: Phenomenex Synergi Polar-RP 100*25 mm*4 μm; mobile phase: [water(TFA)-ACN]; B %: 34%-54%, 7 min) and re-purified by prep-HPLC (column: Waters Xbridge 150*25 mm*5 μm; mobile phase: [water(NH₄HCO₃)-ACN]; B %: 40%-70%, 9 min), the eluent was lyophilized to afford 4-((7,7-difluoro-9-isopropyl-5-methyl-6-oxo-6,7,8,9-tetrahydro-5H-pyrimido[4,5-b][1, 4]diazepin-2-yl)amino)-N-(4-(7-(2-(2,6-dioxopiperidin-3-yl)-6-fluoro-1,3-dioxoisoindolin-5-yl)-2,7-diazaspiro[3.5]nonan-2-yl)piperidin-1-yl)-2-fluoro-5-methoxybenzamide (10.6 mg, 11.06 μmol, 6.08% yield, 96% purity) as a yellow solid. MS(M+H)⁺=920.2

¹H NMR (400 MHz, DMSO-d₆) δ=11.10 (s, 1H), 9.03 (s, 1H), 8.31-8.22 (m, 2H), 7.95 (s, 1H), 7.70 (d, J=11.4 Hz, 1H), 7.44 (br d, J=7.3 Hz, 1H), 7.13 (d, J=6.5 Hz, 1H), 5.10 (br dd, J=5.4, 12.7 Hz, 1H), 4.89 (td, J=6.8, 13.4 Hz, 1H), 4.11-4.00 (m, 2H), 3.91 (s, 3H), 3.33 (br s, 3H), 3.18 (br s, 4H), 2.98 (br s, 6H), 2.93-2.82 (m, 3H), 2.61 (br s, 2H), 2.09 (br d, J=12.2 Hz, 1H), 2.06-1.99 (m, 1H), 1.82 (br s, 4H), 1.72-1.64 (m, 2H), 1.38-1.29 (m, 2H), 1.26 (br d, J=6.6 Hz, 6H).

### Example 218. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(2-(2,6-dioxopiperidin-3-yl)-1-oxoisoindolin-4-yl)-2,7-diazaspiro[3.5]nonan-2-yl)piperidin-1-yl)-3-methoxybenzamide (Compound 218)

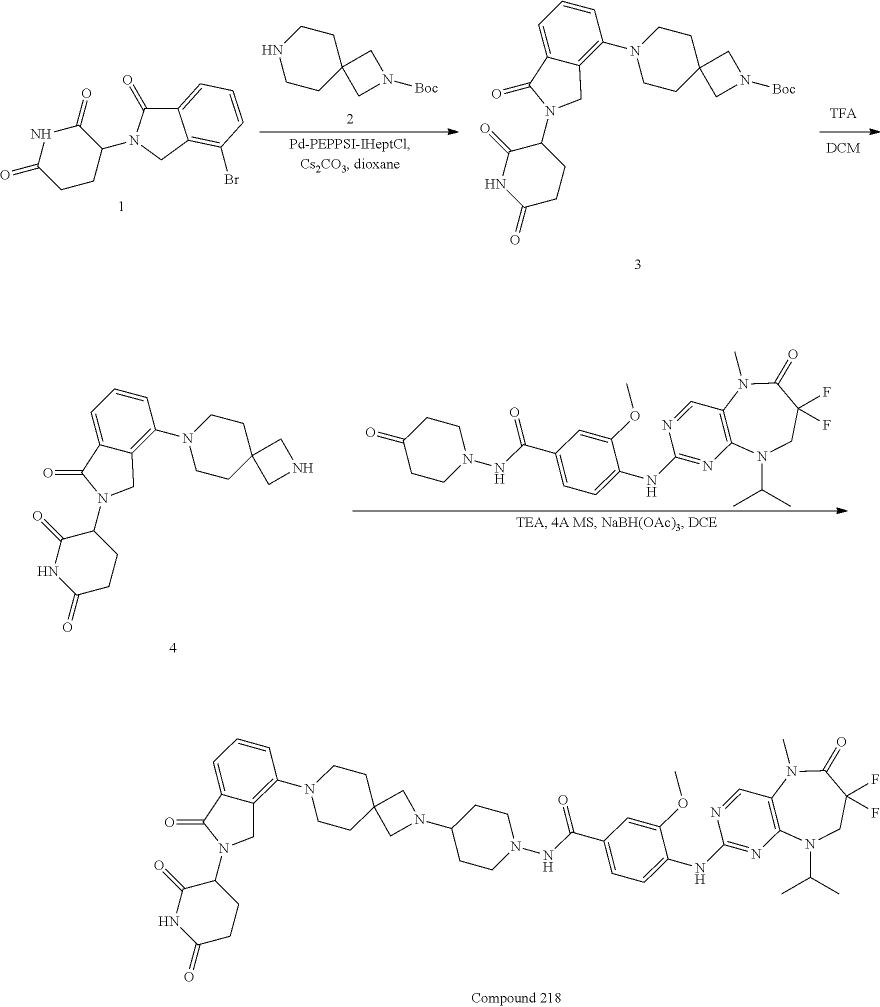 [SMILES: O=C1CCC(N2Cc3c(Br)cccc3C2=O)C(=O)N1 ; CC(C)(C)OC(=O)N1CC2(CCNCC2)C1 ; CC(C)(C)OC(=O)N1CC2(CCN(c3cccc4c3CN(C3CCC(=O)NC3=O)C4=O)CC2)C1 ; O=C1CCC(N2Cc3c(cccc3N3CCC4(CC3)CNC4)C2=O)C(=O)N1 ; COc1cc(C(=O)NN2CCC(=O)CC2)ccc1Nc1ncc2c(n1)N(C(C)C)CC(F)(F)C(=O)N2C ; COc1cc(C(=O)NN2CCC(N3CC4(CCN(c5cccc6c5CN(C5CCC(=O)NC5=O)C6=O)CC4)C3)CC2)ccc1Nc1ncc2c(n1)N(C(C)C)CC(F)(F)C(=O)N2C]

### Step 1. Synthesis of tert-butyl 7-(2-(2,6-dioxopiperidin-3-yl)-1-oxoisoindolin-4-yl)-2,7-diazaspiro[3.5]nonane-2-carboxylate (3)

To a solution of 3-(4-bromo-1-oxoisoindolin-2-yl)piperidine-2,6-dione (0.3 g, 928.39 μmol) and tert-butyl 2,7-diazaspiro[3.5]nonane-2-carboxylate (318.00 mg, 1.41 mmol) in dioxane (8 mL) were added Cs₂CO₃ (907.46 mg, 2.79 mmol) and Pd-PEPPSI-IHeptCl (27.09 mg, 27.85 μmol). The reaction mixture was stirred at 100° C. for 28 h. TLC (Petroleum ether:EtOAc=10:1) showed new spot was formed. The mixture was filtered and the filter cake was washed with MeCN (10 mL) and DCM (10 mL). The filtrate was concentrated under reduced pressure. The residue was purified by column chromatography (SiO₂, Petroleum ether/EtOAc=20/1 to 0/1), then re-purified by prep-TLC (SiO₂, Petroleum ether:EtOAc=10:1) to afford tert-butyl 7-(2-(2,6-dioxopiperidin-3-yl)-1-oxoisoindolin-4-yl)-2,7-diazaspiro[3.5]nonane-2-carboxylate (30 mg, 54.42 μmol, 1.47% yield, 85% purity) as a yellow solid. MS(M+H)⁺=469.2

### Step 2. Synthesis of 3-(1-oxo-4-(2,7-diazaspiro[3.5]nonan-7-yl)isoindolin-2-yl)piperidine-2,6-dione (4)

To a solution of tert-butyl 7-(2-(2,6-dioxopiperidin-3-yl)-1-oxoisoindolin-4-yl)-2,7-diazaspiro[3.5]nonane-2-carboxylate (50 mg, 106.71 μmol) in DCM (0.3 mL) was added TFA (154.00 mg, 1.35 mmol, 0.1 mL) at 0° C. and the mixture was stirred at 20° C. for 1 h. LCMS showed the starting material was consumed and the desired mass was detected. The mixture was concentrated under reduced pressure to afford 3-(1-oxo-4-(2,7-diazaspiro[3.5]nonan-7-yl)isoindolin-2-yl)piperidine-2,6-dione (50 mg, crude, TFA salt) as a yellow oil. MS(M+H)⁺=369.4

### Step 3. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(2-(2,6-dioxopiperidin-3-yl)-1-oxoisoindolin-4-yl)-2,7-diazaspiro[3.5]nonan-2-yl)piperidin-1-yl)-3-methoxybenzamide (Compound 218)

To a solution of 3-(1-oxo-4-(2,7-diazaspiro[3.5]nonan-7-yl)isoindolin-2-yl)piperidine-2,6-dione (50 mg, 103.64 μmol, TFA salt) in DCE (1 mL) were added TEA (109.05 mg, 1.08 mmol, 150 μL), 4A MS (0.1 g) and 4-((7,7-difluoro-9-isopropyl-5-methyl-6-oxo-6,7,8,9-tetrahydro-5H-pyrimido[4,5-b][1, 4]diazepin-2-yl)amino)-3-methoxy-N-(4-oxopiperidin-1-yl)benzamide (60 mg, 115.94 μmol) at 0° C. and the mixture was stirred at 20° C. for 0.5 h. NaBH(OAc)₃ (65.90 mg, 310.91 μmol) was added and the mixture was stirred at 20° C. for 3 h. LCMS showed 35% of the desired mass. The mixture was filtered and the filter cake was washed with DCM (10 mL). The filtrate was concentrated under reduced pressure. The crude was purified by flash silica gel chromatography (5 g SepaFlash® Silica Flash Column, Eluent of 30˜40% MeOH/EtOAc gradient @50 mL/min), then re-purified by prep-HPLC (column: Phenomenex Synergi Polar-RP 100*25 mm*4 um; mobile phase: [water(TFA)-ACN]; B %: 27%-47%, 10 min) and the eluent was lyophilized to afford 4-((7,7-difluoro-9-isopropyl-5-methyl-6-oxo-6,7,8,9-tetrahydro-5H-pyrimido[4,5-b][1, 4]diazepin-2-yl)amino)-N-(4-(7-(2-(2,6-dioxopiperidin-3-yl)-1-oxoisoindolin-4-yl)-2,7-diazaspiro[3.5]nonan-2-yl)piperidin-1-yl)-3-methoxybenzamide (17.9 mg, 14.67 μmol, 14.16% yield, 90% purity, 2 TFA) as a white solid. MS(M+H)⁺=870.2

¹H NMR (400 MHz, DMSO-d₆) δ=11.00 (s, 1H), 10.06-9.94 (m, 1H), 9.50 (s, 1H), 8.26 (br d, J=8.1 Hz, 1H), 8.23 (s, 1H), 7.47-7.42 (m, 3H), 7.33 (d, J=7.5 Hz, 1H), 7.18 (d, J=7.8 Hz, 1H), 5.13 (dd, J=5.3, 13.3 Hz, 1H), 4.90-4.85 (m, 1H), 4.48-4.42 (m, 1H), 4.32-4.27 (m, 1H), 4.11-4.06 (m, 1H), 4.05-4.02 (m, 3H), 4.01-3.96 (m, 2H), 3.93 (s, 3H), 3.32 (s, 3H), 3.14-3.05 (m, 4H), 2.99-2.81 (m, 5H), 2.65-2.57 (m, 2H), 2.46-2.41 (m, 1H), 2.05-1.95 (m, 5H), 1.93-1.85 (m, 2H), 1.56-1.43 (m, 2H), 1.24 (d, J=6.7 Hz, 6H).

### Example 219. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(2-(2,6-dioxopiperidin-3-yl)-1-oxoisoindolin-5-yl)-2,7-diazaspiro[3.5]nonan-2-yl)piperidin-1-yl)-3-methoxybenzamide (Compound 219)

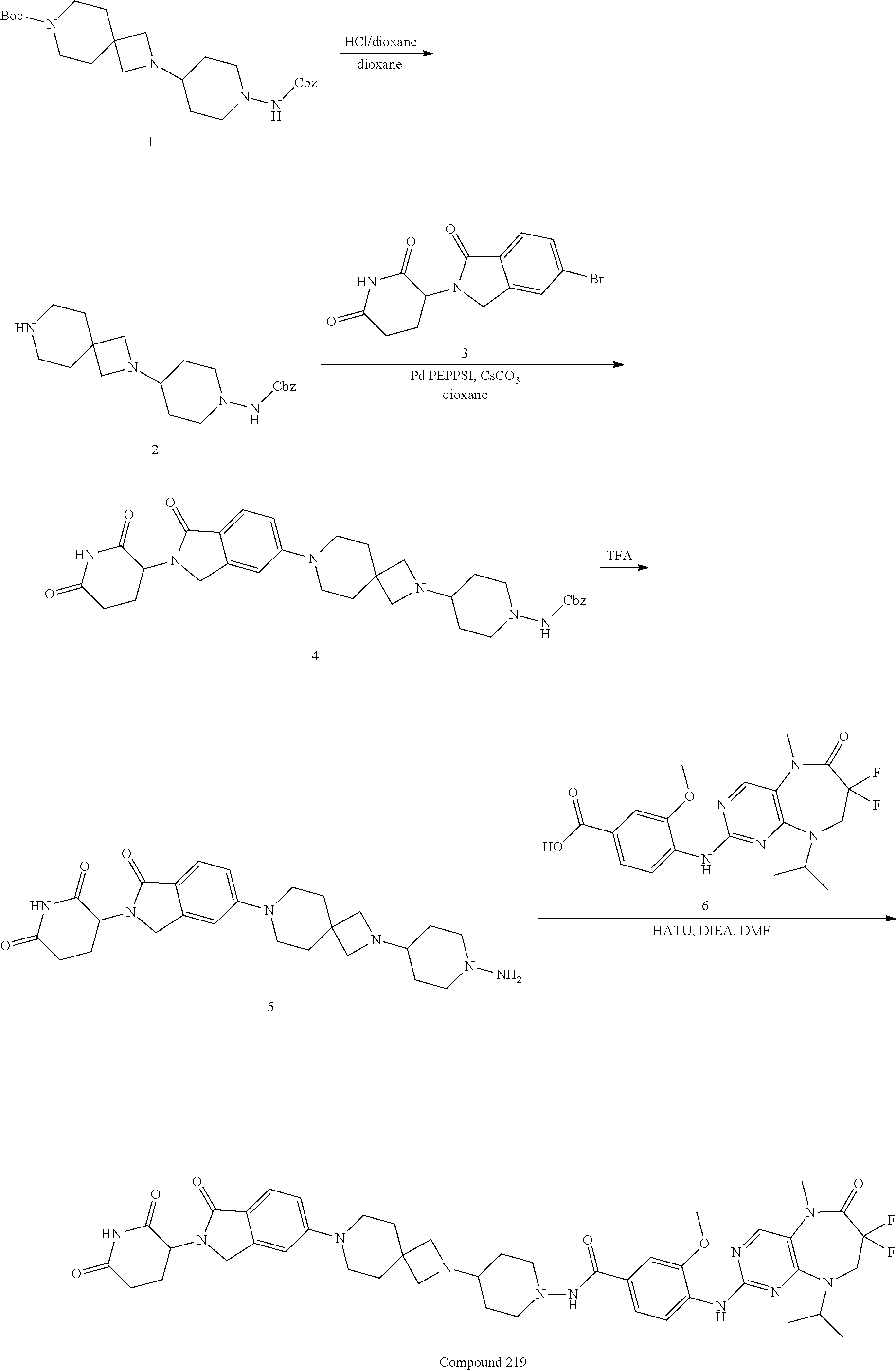

The compound 219 was synthesized by the method described in the scheme similar to the method described in Example 211.

### EtOAcEtOAc

MS(M+H)⁺=871.0, ¹H NMR (400 MHz, DMSO-d₆) δ=10.95 (s, 1H), 9.98-9.82 (m, 1H), 9.50 (br s, 1H), 8.29-8.14 (m, 2H), 7.52 (d, J=8.4 Hz, 1H), 7.46-7.40 (m, 2H), 7.14-7.05 (m, 2H), 5.08-5.02 (m, 1H), 4.92-4.85 (m, 1H), 4.35-4.29 (m, 1H), 4.22-4.18 (m, 1H), 4.12-4.07 (m, 1H), 4.06-4.01 (m, 3H), 4.00-3.96 (m, 2H), 3.93 (s, 3H), 3.42-3.37 (m, 2H), 3.32 (s, 3H), 3.30-3.25 (m, 2H), 3.13-3.06 (m, 2H), 2.96-2.80 (m, 3H), 2.62-2.55 (m, 2H), 2.41-2.34 (m, 1H), 2.02-1.88 (m, 5H), 1.87-1.80 (m, 2H), 1.55-1.40 (m, 2H), 1.24 (d, J=6.7 Hz, 6H).

### Example 220. Synthesis of 4-((7,7-difluoro-9-isopropyl-5-methyl-6-oxo-6,7,8,9-tetrahydro-5H-pyrimido[4,5-b][1,4]diazepin-2-yl)amino)-N-(4-(7-(2-(2,6-dioxopiperidin-3-yl)-1-oxoisoindolin-5-yl)-2,7-diazaspiro[3.5]nonan-2-yl)piperidin-1-yl)-2-fluoro-5-methoxybenzamide (Compound 220)

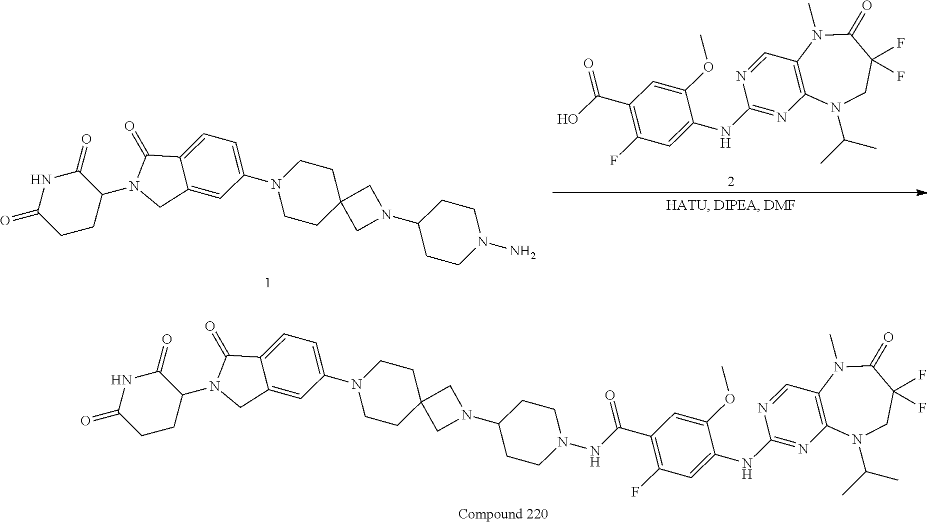 [SMILES: NN1CCC(N2CC3(CCN(c4ccc5c(c4)CN(C4CCC(=O)NC4=O)C5=O)CC3)C2)CC1 ; COc1cc(C(=O)O)c(F)cc1Nc1ncc2c(n1)N(C(C)C)CC(F)(F)C(=O)N2C ; COc1cc(C(=O)NN2CCC(N3CC4(CCN(c5ccc6c(c5)CN(C5CCC(=O)NC5=O)C6=O)CC4)C3)CC2)c(F)cc1Nc1ncc2c(n1)N(C(C)C)CC(F)(F)C(=O)N2C]

The compound 220 was synthesized by the method described in the scheme similar to the method described in Example 211.

MS(M+H)⁺=888.5, ¹H NMR (400 MHz, DMSO-d₆) δ=10.94 (s, 1H), 10.01-9.81 (m, 1H), 8.29-8.21 (m, 2H), 8.17-8.01 (m, 1H), 7.52 (d, J=8.4 Hz, 1H), 7.14 (d, J=6.5 Hz, 1H), 7.12-7.06 (m, 2H), 5.08-5.01 (m, 1H), 4.93-4.86 (m, 1H), 4.35-4.30 (m, 1H), 4.22-4.18 (m, 1H), 4.12-4.08 (m, 1H), 4.05-3.93 (m, 5H), 3.92-3.87 (m, 3H), 3.42-3.36 (m, 2H), 3.33 (s, 3H), 3.29-3.24 (m, 2H), 3.14-3.07 (m, 2H), 2.96-2.84 (m, 1H), 2.80-2.69 (m, 2H), 2.62-2.54 (m, 2H), 2.39-2.34 (m, 1H), 2.02-1.88 (m, 5H), 1.87-1.80 (m, 2H), 1.55-1.39 (m, 2H), 1.26 (d, J=6.6 Hz, 6H).

### Example 221. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(2-(2-((2-(2,6-dioxopiperidin-3-yl)-1,3-dioxoisoindolin-4-yl)amino)ethoxy)ethyl)-5-methoxy-2-(4-methylpiperazin-1-yl)benzamide (Compound 221)

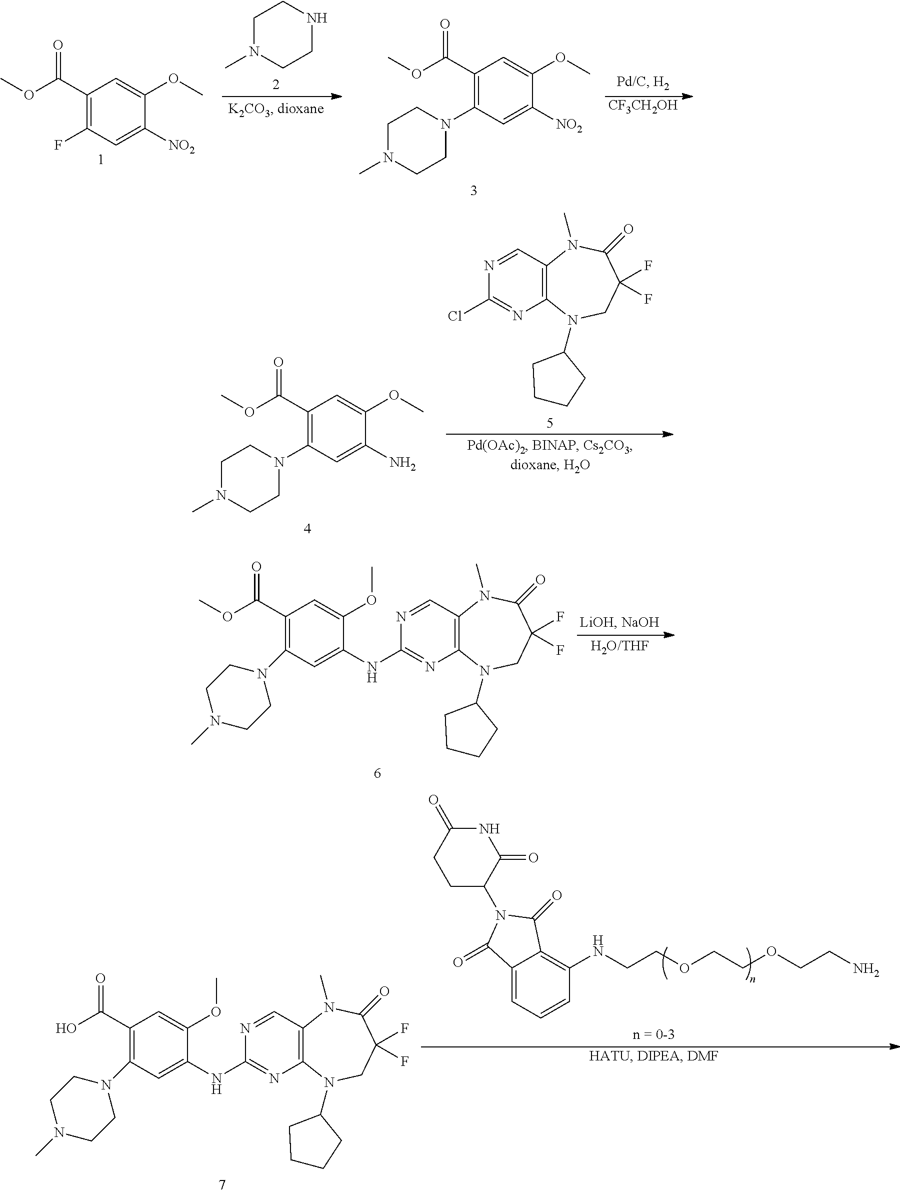

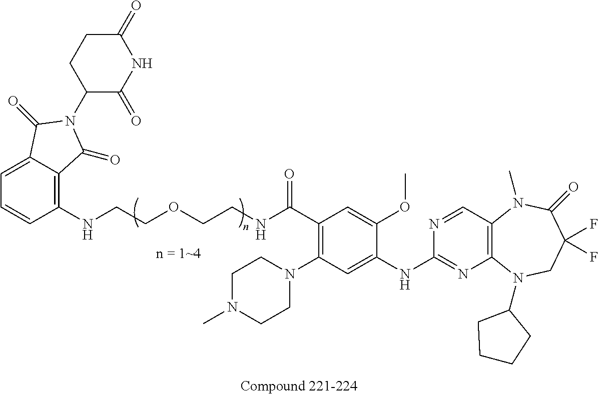

### Step 1. Synthesis of methyl 5-methoxy-2-(4-methylpiperazin-1-yl)-4-nitrobenzoate (3)

A mixture of methyl 2-fluoro-5-methoxy-4-nitrobenzoate (4 g, 17.45 mmol), 1-methylpiperazine (3.50 g, 34.91 mmol, 3.87 mL) and K₂CO₃ (7.24 g, 52.36 mmol) in dioxane (60 mL) was stirred 100° C. for 12 hr. LCMS showed a peak (66%) with desired mass. TLC (petroleum ether:EtOAc=3:1; Rf=0.7) showed the starting material remained. The mixture was filtered and the filter cake was washed with Dichloromethane (50 mL). The filtrate was concentrated under reduced pressure. The residue was purified by flash silica gel chromatography (25 g SepaFlash® Silica Flash Column, Eluent of 0˜50% EtOAc/Petroleum ether gradient @65 mL/min) to afford methyl 5-methoxy-2-(4-methylpiperazin-1-yl)-4-nitrobenzoate (2.7 g, 8.64 mmol, 49.51% yield, 99% purity) as brown oil, which was used for the next step directly. MS (M+H)⁺=310.1

### Step 2. Synthesis of methyl 4-amino-5-methoxy-2-(4-methylpiperazin-1-yl)benzoate (4)

To a solution of methyl 5-methoxy-2-(4-methylpiperazin-1-yl)-4-nitrobenzoate (2.7 g, 8.73 mmol) in CF₃CH₂OH (80 mL) was added Pd/C (500 mg, 10% purity) under N₂ atmosphere. The mixture was stirred at 30° C. under H₂ (15 psi) for 12 hr. LCMS showed the starting material was consumed completely and a main peak with desired mass. The reaction mixture was filtered through a celite pad and the filtrate was concentrated. The crude product was diluted with deionized water (20 mL) and MeCN (10 mL) and lyophilized to afford methyl 4-amino-5-methoxy-2-(4-methylpiperazin-1-yl)benzoate (2.4 g, 8.25 mmol, 94.49% yield, 96% purity) as a white solid, which was used for the next step directly. MS(M+H)⁺=280.1

¹H NMR (400 MHz, DMSO-d₆) δ=7.19 (s, 1H), 6.36 (s, 1H), 5.49 (s, 2H), 3.70 (d, J=10.9 Hz, 6H), 2.83 (br s, 4H), 2.43 (br s, 4H), 2.20 (s, 3H)

### Step 3. Synthesis of methyl 4-((9-cyclopentyl-7,7-difluoro-5-methyl-6-oxo-6,7,8,9-tetrahydro-5H-pyrimido[4,5-b][1,4]diazepin-2-yl)amino)-5-methoxy-2-(4-methylpiperazin-1-yl)benzoate (6)

To a solution of methyl 4-amino-5-methoxy-2-(4-methylpiperazin-1-yl)benzoate (0.5 g, 1.58 mmol), 2-chloro-9-cyclopentyl-7,7-difluoro-5-methyl-8,9-dihydro-5H-pyrimido[4,5-b][1,4]diazepin-6(7H)-one (485.06 mg, 1.74 mmol) in dioxane (8 mL) were added Cs₂CO₃ (1.54 g, 4.74 mmol), BINAP (196.59 mg, 315.72 μmol) and Pd(OAc)₂ (35.44 mg, 157.86 μmol) at 25° C. The mixture was stirred at 100° C. for 16 hr under N₂ atmosphere. LCMS showed the 2-chloro-9-cyclopentyl-7,7-difluoro-5-methyl-8,9-dihydro-5H-pyrimido[4,5-b][1,4]diazepin-6(7H)-one was consumed completely, and a main peak with desired mass. The reaction mixture was filtered through a celite pad and the filtrate was concentrated. The residue was purified by flash silica gel chromatography (25 g SepaFlash® Silica Flash Column, Eluent of 0˜100% EtOAc/Petroleum ether gradient @80 mL/min; Eluent of 0˜50% Methanol/EtOAc @80 mL/min) followed by prep-HPLC (column: Waters Xbridge C₁₈ 150*50 mm*10 um; mobile phase: [water (10 mM NH₄HCO₃)-ACN]; B %: 44%-74%, 11 min) and the eluent was lyophilized to afford methyl 4-((9-cyclopentyl-7,7-difluoro-5-methyl-6-oxo-6,7,8,9-tetrahydro-5H-pyrimido[4,5-b][1,4]diazepin-2-yl)amino)-5-methoxy-2-(4-methylpiperazin-1-yl)benzoate (0.5 g, 866.68 μmol, 54.90% yield, 97% purity) as a white solid, which was used for the next step directly. MS (M+H)⁺=560.3

¹H NMR (400 MHz, CDCl₃) δ=8.35 (s, 1H), 8.09 (s, 1H), 7.73 (s, 1H), 7.42 (s, 1H), 4.79 (quin, J=8.3 Hz, 1H), 3.97-3.89 (m, 4H), 3.89-3.83 (m, 4H), 3.41 (s, 3H), 3.11 (br t, J=4.4 Hz, 4H), 2.66 (br s, 4H), 2.39 (s, 3H), 2.16-2.06 (m, 2H), 1.80-1.60 (m, 4H), 1.60-1.46 (m, 2H)

### Step 4. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-5-methoxy-2-(4-methylpiperazin-1-yl)benzoic acid (7)

A mixture of methyl 4-((9-cyclopentyl-7,7-difluoro-5-methyl-6-oxo-6,7,8,9-tetrahydro-5H-pyrimido[4,5-b][1,4]diazepin-2-yl)amino)-5-methoxy-2-(4-methylpiperazin-1-yl)benzoate (0.45 g, 804.14 μmol), LiOH (23.11 mg, 964.96 μmol) in THF (5 mL) and H₂O (5 mL) was stirred at 25° C. for 12 hr. LCMS showed the starting material (30%) remained, and a main peak (65%) with desired mass. Additional NaOH (321.63 mg, 8.04 mmol) was added at 25° C. and the resulting solution was stirred at 40° C. for another 24 hr. LCMS showed the starting material was consumed completely, and a main peak with desired mass. The mixture was concentrated under reduced pressure. The crude product was purified by prep-HPLC (column: Phenomenex luna C₁₈ 150*40 mm*15 um; mobile phase: [water (0.1% TFA)-ACN]; B %: 9%-39%, 11 min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-5-methoxy-2-(4-methylpiperazin-1-yl)benzoic acid (140 mg, 212.98 μmol, 23.24% yield, 83% purity) as a white solid, which was used for the next step directly. MS(M+H)⁺=546.3

### Step 5. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(2-(2-((2-(2,6-dioxopiperidin-3-yl)-1,3-dioxoisoindolin-4-yl)amino)ethoxy)ethyl)-5-methoxy-2-(4-methylpiperazin-1-yl)benzamide (Compound 221)

To a solution of 4-((9-cyclopentyl-7,7-difluoro-5-methyl-6-oxo-6,7,8,9-tetrahydro-5H-pyrimido[4,5-b][1,4]diazepin-2-yl)amino)-5-methoxy-2-(4-methylpiperazin-1-yl)benzoic acid (150 mg, 274.94 μmol) in DMF (3 mL) were added HATU (156.81 mg, 412.40 μmol) and DIPEA (106.60 mg, 824.81 μmol, 143.66 μL) and the mixture was stirred at 30° C. for 10 min. Then 4-((2-(2-aminoethoxy)ethyl)amino)-2-(2,6-dioxopiperidin-3-yl)isoindoline-1,3-dione (54.55 mg, 137.47 μmol, HCl salt) was added and the resulting mixture was stirred at 30° C. for 12 h. LCMS showed the 4-((2-(2-aminoethoxy)ethyl)amino)-2-(2,6-dioxopiperidin-3-yl)isoindoline-1,3-dione was consumed completely and a peak (49%) with desired mass. The mixture was concentrated under reduced pressure. The crude product was purified by prep-HPLC (column: Phenomenex Synergi Polar-RP 100*25 mm*4 um; mobile phase: [water (0.1% TFA)-ACN]; B %: 35%-55%, 7 min) and followed by prep-HPLC (column: Waters Xbridge 150*25 mm*5 um; mobile phase: [water (10 mM NH₄HCO₃)-ACN]; B %: 42%-72%, min) and the eluent was lyophilized to afford 4-((9-cyclopentyl-7,7-difluoro-5-methyl-6-oxo-6,7,8,9-tetrahydro-5H-pyrimido[4,5-b][1,4]diazepin-2-yl)amino)-N-(2-(2-((2-(2,6-dioxopiperidin-3-yl)-1,3-dioxoisoindolin-4-yl)amino)ethoxy)ethyl)-5-methoxy-2-(4-methylpiperazin-1-yl)benzamide (13.4 mg, 14.94 μmol, 5.43% yield, 99% purity) as a yellow solid. MS(M+H)⁺=888.4

¹H NMR (400 MHz, DMSO-d₆) δ=11.06 (s, 1H), 10.40-10.30 (m, 1H), 8.30 (s, 1H), 8.27 (s, 1H), 7.94 (s, 1H), 7.60 (s, 1H), 7.56 (t, J=7.9 Hz, 1H), 7.14 (d, J=8.4 Hz, 1H), 7.02 (d, J=7.1 Hz, 1H), 6.63 (br t, J=5.5 Hz, 1H), 5.00 (dd, J=5.4, 12.8 Hz, 1H), 4.93-4.81 (m, 1H), 4.03 (br t, J=14.1 Hz, 2H), 3.89 (s, 3H), 3.74-3.67 (m, 2H), 3.66-3.59 (m, 2H), 3.57-3.48 (m, 4H), 3.33 (br s, 3H), 2.82 (br s, 4H), 2.49-2.32 (m, 7H), 2.14 (s, 3H), 2.05-1.88 (m, 3H), 1.73-1.45 (m, 6H).

### Example 222. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(2-(2-(2-((2-(2,6-dioxopiperidin-3-yl)-1,3-dioxoisoindolin-4-yl)amino)ethoxy)ethoxy)ethyl)-5-methoxy-2-(4-methylpiperazin-1-yl)benzamide (Compound 222)

The compound 222 was synthesized by the method described in the scheme similar to the method described in Example 221.

MS(M+H)⁺=932.5, ¹H NMR (400 MHz, DMSO-d₆) δ=11.08 (s, 1H), 10.31 (br t, J=5.3 Hz, 1H), 8.30 (s, 1H), 8.27 (s, 1H), 7.93 (s, 1H), 7.60 (s, 1H), 7.54 (dd, J=7.2, 8.4 Hz, 1H), 7.10 (d, J=8.6 Hz, 1H), 7.01 (d, J=6.9 Hz, 1H), 6.59 (t, J=5.7 Hz, 1H), 5.03 (dd, J=5.4, 12.9 Hz, 1H), 4.87 (quin, J=8.1 Hz, 1H), 4.04 (t, J=14.0 Hz, 2H), 3.89 (s, 3H), 3.66-3.56 (m, 8H), 3.50 (q, J=5.3 Hz, 2H), 3.43 (q, J=5.4 Hz, 2H), 3.33 (br s, 3H), 2.92-2.79 (m, 5H), 2.61-2.51 (m, 3H), 2.49-2.42 (m, 3H), 2.22 (s, 3H), 2.06-1.90 (m, 3H), 1.73-1.48 (m, 6H).

### Example 223. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(2-(2-(2-(2-((2-(2,6-dioxopiperidin-3-yl)-1,3-dioxoisoindolin-4-yl)amino)ethoxy)ethoxy)ethoxy)ethyl)-5-methoxy-2-(4-methylpiperazin-1-yl)benzamide (Compound 223)

The compound 223 was synthesized by the method described in the scheme similar to the method described in Example 221.

MS(M+H)⁺=976.6, ¹H NMR (400 MHz, DMSO-d₆) δ=11.09 (s, 1H), 10.32 (t, J=5.3 Hz, 1H), 8.32-8.25 (m, 2H), 7.93 (s, 1H), 7.61 (s, 1H), 7.55 (dd, J=7.2, 8.4 Hz, 1H), 7.10 (d, J=8.6 Hz, 1H), 7.01 (d, J=7.0 Hz, 1H), 6.58 (t, J=5.8 Hz, 1H), 5.04 (dd, J=5.4, 12.9 Hz, 1H), 4.94-4.81 (m, 1H), 4.04 (t, J=14.1 Hz, 2H), 3.89 (s, 3H), 3.63-3.46 (m, 14H), 3.42 (q, J=5.6 Hz, 2H), 3.30 (br s, 3H), 2.94-2.81 (m, 5H), 2.62-2.51 (m, 4H), 2.46 (br d, J=4.3 Hz, 2H), 2.23 (s, 3H), 2.07-1.91 (m, 3H), 1.76-1.47 (m, 6H).

### Example 224. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(14-((2-(2,6-dioxopiperidin-3-yl)-1,3-dioxoisoindolin-4-yl)amino)-3,6,9,12-tetraoxatetradecyl)-5-methoxy-2-(4-methylpiperazin-1-yl)benzamide (Compound 224)

The compound 224 was synthesized by the method described in the scheme similar to the method described in Example 221.

MS(M+H)⁺=1020.6, ¹H NMR (400 MHz, DMSO-d₆) δ=11.09 (s, 1H), 10.38-10.21 (m, 1H), 8.33-8.24 (m, 2H), 7.94 (s, 1H), 7.60 (s, 1H), 7.56 (dd, J=7.2, 8.4 Hz, 1H), 7.11 (d, J=8.5 Hz, 1H), 7.02 (d, J=7.0 Hz, 1H), 6.58 (t, J=5.8 Hz, 1H), 5.05 (dd, J=5.4, 12.9 Hz, 1H), 4.87 (br t, J=8.1 Hz, 1H), 4.04 (t, J=14.1 Hz, 2H), 3.89 (s, 3H), 3.62-3.47 (m, 18H), 3.43 (q, J=5.5 Hz, 2H), 3.32-3.29 (m, 3H), 2.95-2.81 (m, 5H), 2.63-2.51 (m, 4H), 2.49-2.42 (m, 2H), 2.36-2.16 (m, 3H), 2.07-1.91 (m, 3H), 1.75-1.47 (m, 6H).

### Example 225. Synthesis of 4-((9-cyclopentyl-7,7-difluoro-5-methyl-6-oxo-6,7,8,9-tetrahydro-5H-pyrimido[4,5-b][1,4]diazepin-2-yl)amino)-N-(17-((2-(2,6-dioxopiperidin-3-yl)-1,3-dioxoisoindolin-4-yl)amino)-3,6,9,12,15-pentaoxaheptadecyl)-5-methoxy-2-(4-methylpiperazin-1-yl)benzamide (Compound 225)

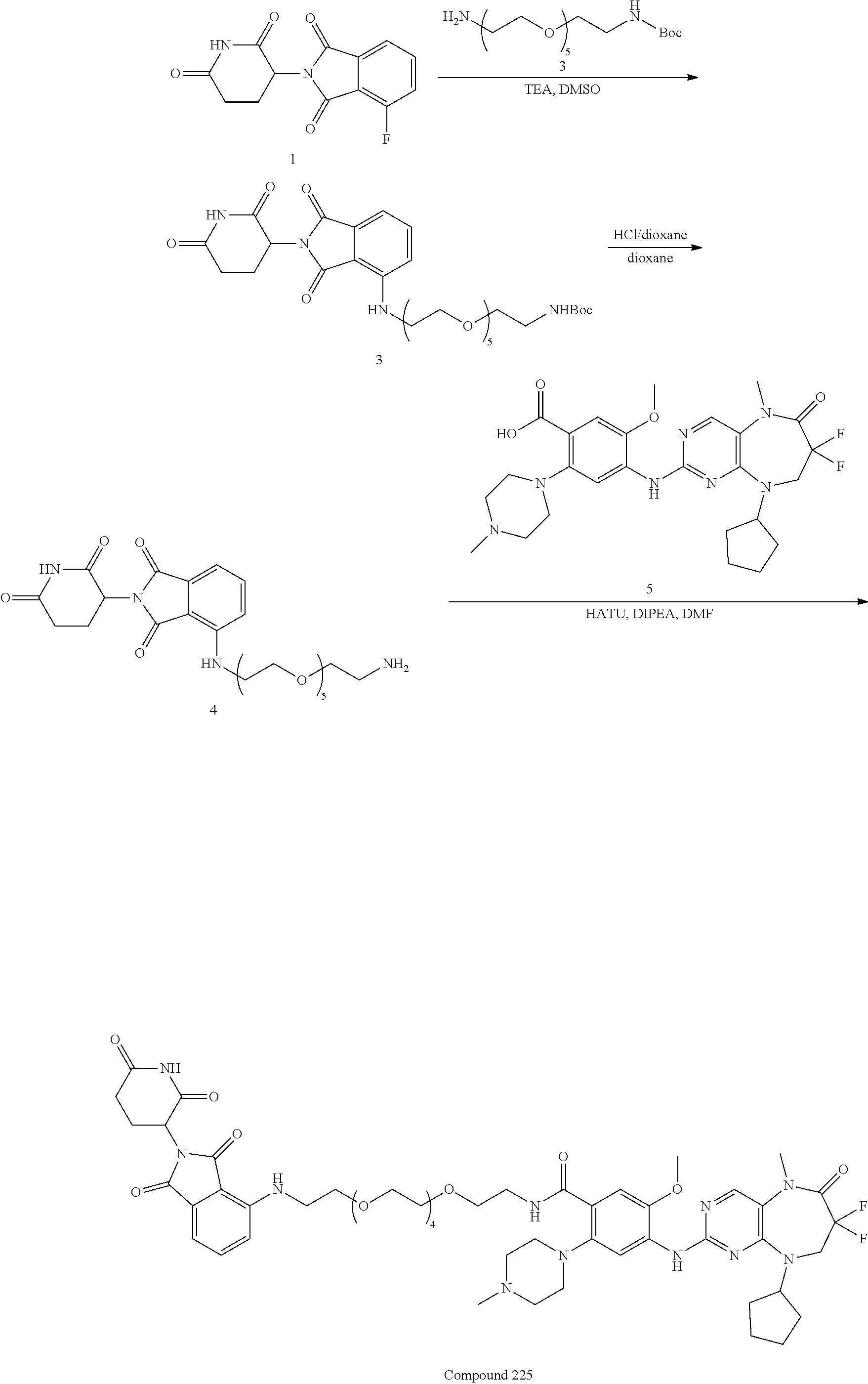

The compound 225 was synthesized by the method described in the scheme similar to the method described in Example 221.

MS(M+H)⁺=1064.7, ¹H NMR (400 MHz, DMSO-d₆) δ=11.09 (s, 1H), 10.32 (br s, 1H), 8.34-8.24 (m, 2H), 7.94 (s, 1H), 7.61 (s, 1H), 7.56 (dd, J=7.2, 8.4 Hz, 1H), 7.12 (d, J=8.6 Hz, 1H), 7.03 (d, J=7.0 Hz, 1H), 6.59 (t, J=5.8 Hz, 1H), 5.05 (dd, J=5.4, 12.9 Hz, 1H), 4.94-4.82 (m, 1H), 4.04 (t, J=14.0 Hz, 2H), 3.89 (s, 3H), 3.61-3.42 (m, 24H), 3.30 (br s, 3H), 2.95-2.79 (m, 5H), 2.63-2.52 (m, 4H), 2.49-2.40 (m, 2H), 2.25 (br s, 3H), 2.07-1.91 (m, 3H), 1.75-1.47 (m, 6H).

### Experimental Examples

### 1. Western Blot Assay for PLK1

### (1) Culture of HeLa Cell Line

The HeLa cell line was purchased from Korea Cell Line Bank (KCLB), Seoul, Korea. The passage in cell culture was maintained at P115 to P125.

For cell counting, cell counter (Thermo Fisher Scientific Inc., Catalog #AMQAX1000) and 0.4% trypan blue solution were used.

For cell culture, DMEM (Gibco, Cat. No. 1195-65; Lot. No. 2085318), FBS (Gibco, Cat. No. 16000-044; Lot. No. 2097593), Penicillin/Streptomycin (PS) (Gibco, Cat. No. 15140-122; Lot. No. 2058855), 100 mm² cell culture dish (SPL, Cat. No. 20100), 150 mm² cell culture dish (SPL, Cat. No. 20150), 12-well culture plate (SPL, Cat. No. 30012), PBS pH 7.4 (Gibco, Cat. No. 10010-023; Lot. No. 2085080), TrypLE™ Express (Gibco, Cat. No. 12605-010; Lot No. 2070638), Counting Chamber (Hematocytometer) (Hirschmann, Cat. No. 8100204), and 0.4% Trypan Blue Solution (DYNEBIO, Cat. No. CBT3710; Lot. No. 20190723) were used.

### (2) Treatment of Compounds of the Present Invention

2×10⁵ cells were seeded for each well of a 12-well plate (SPL), and the cells were cultured in the culture medium in a total volume of 2 mL.

The compounds of Examples were completely dissolved in DMSO and used in the experiment, and thymidine was completely dissolved in DW and used in the experiment. For thymidine block, the products were treated with 2 mM of thymidine (Sigma-Aldrich Cat. No. T9250-5G) and then incubated for 24 hours.

For release and chemical treatment, the medium was suctioned and washed 3 times with 1×PBS. Complete media was added, followed by incubation for 4 hours in a CO₂ incubator. Each compound was diluted three folds from the highest concentration of 3 μM to the lowest concentration and then incubated for 6 hours again.

### (3) Western Blotting

For SDS-PAGE and Western blotting, 1×RIPA lysis buffer (Rockland, Cat. No. MB-030-0050; Lot no. 39751), 100× Protease Inhibitor Cocktail (Quartett, Cat. No. PPI1015; Lot no. PC050038424), Pierce™ BCA protein assay kit (ThermoScientific, Cat. No. 23225; Lot no. UC276876), albumin standard (ThermoScientific, Cat. No. 23209; Lot no. UB269561), 4-15% Mini-PROTEAN TGX stain-free gel (Bio-rad, Cat. No. 4568085; Lot no. L007041B), 10× Tris/Glycine/SDS buffer (Bio-rad, Cat. No. 1610732; Lot no. 10000044375B); 10×TBS (Bio-rad, Cat. No. 1706435; Lot no. 1000045140B), 10% Tween 20 (Cat. No. 1610781; Lot no. L004152B), Color protein standard broad range (NEB, Cat. No. P7719S; Lot no. 10040349), 4× Laemmli sample buffer (Bio-rad, Cat. No. 1610747; Lot no. L004133B), β-mercaptoethanol (Sigma-Aldrich, Cat. No. M3148; Lot no. 60-24-2), SuperBlock™ T20 (TBS) blocking buffer (ThermoScientific, Cat. No. 37536; Lot no. UC282578), 1 M sodium azide solution (Sigma-Aldrich, Cat. No. 08591-1 mL-F; Lot no. BCBV4989), α-Rabbit pAb to Ms IgG (abcam, Cat. No. ab97046; Lot no. GR3252115-1), α-Goat pAb to Rb IgG (CST, Cat. No. 7074S; Lot no. 28), α-GAPDH (abeam, Cat. No. ab8245; Lot no. GR3275542-2), α-PLK1 (CST, Cat. No. 208G4), α-BRD4 (CST, Cat. No. 13440S), ECL™ Prime western blotting reagents (GE Healthcare, Cat. No. RPN2232; Lot no. 17001655), Ponceau S solution (Sigma-Aldrich, Cat. No. P7170; Lot no. SLBV4112), Difco™ Skim milk (BD, Cat. No. 232100; Lot no. 8346795), and iBlot® 2 NC Regular stacks (Invitrogen, Cat. No. IB23001; Lot no. 2NR110619-02) were used.

For cell harvesting, the cells were first separated from the plate using trypsin and then washed with the medium and PBS. Specifically, the medium was suctioned off and washed with 1 mL of PBS, and PBS was suctioned off. The cells were treated with 0.5 mL TrypLE™ Express at 37° C. for 7 minutes to separate the cells, and then 0.5 mL of complete medium was added to collect 1 mL of cell culture solution. Then, 1 mL of the cell collection solution was centrifuged at 8,000 rpm for 120 seconds, and the supernatant was removed. After washing with 0.2 mL of PBS, the PBS was removed.

For cell lysis, a lysis buffer was added and cell debris was removed to obtain a cell lysate. Specifically, the cells were treated with 70 μL of 1×RIPA buffer containing a protease inhibitor and incubated for 30 minutes on ice. Then, the cells were centrifuged at 4° C. and 15,000 rpm for 10 minutes to obtain a cell lysate.

Then, a standard curve was obtained using the BCA assay, and the protein mass in the lysate was quantified by substituting the curve equation. The mixture was incubated at 37° C. for 30 minutes using 20 μL of standard or sample solution, and 200 μL of BCA or Bradford solution, and measured at 562 nm absorbance. Samples were prepared by adding 4× sample buffer so that the quantity of protein added to each well was 15 g.

Sodium dodecyl sulfate polyacrylamide gel electrophoresis (SDS-PAGE) was performed by setting a running time of 100 minutes at 120 V on a 4-15% Mini-PROTEAN TGX stain-free gel (15 well). Transferring was performed on iBlot® 2 NC Mini stacks at P0 mode of the dry blotting system. After staining using Ponceau S solution, blocking was performed for 1 hour with a blocking buffer (Thermo). After washing with 1×TBS containing 0.05% Tween 20, the product was reacted at 4° C. for 16 hours with anti-PLK1 (CST) antibody (1:500), anti-BRD4 (Cell signaling) antibody (1:1000) or anti-GAPDH (abeam) antibody (1:10,000) in 1×TBS-T as a primary antibody. After washing three times for 10 minutes with 1×TBS containing 0.05% Tween20, the product was reacted at room temperature for 1 hour with anti-mouse antibody (abcam) (1:10000) or anti-rabbit antibody (CST) (1:5000) in 1×TBS-T as a secondary antibody. Then, after washing three times for 10 minutes with 1×TBS containing 0.05% Tween 20, the product was detected with an ECL working solution (1:1).

To analyze the results, an image analyzer (GE) was used to obtain final blot data. As a result, it was confirmed that all of the compounds of the present invention degraded PLK1 protein significantly.

### 2. Luciferase Assay for PLK1

### (1) Preparation and Culture of HeLa LgBit (Plk1-HiBit KI) Cell Line

A HeLa cell line in which the LgBit vector was transfected and expressed stably was prepared. Then, after constructing gRNA and donor to express the HiBit amino acid sequence behind the C-terminal of the Plk1 gene, which was inherent in the cell, it was inserted into the cell together with a vector capable of expressing CRISPR/Cas9. Only the cells in which the insertion was completed and knock-in had progressed were selected, sub-cultured and used.

For cell culture, DMEM (Gibco, Cat. No. 11995-065; Lot. No. 2467189), FBS (Gibco, Cat. No. 16000-044; Lot. No. 2420173P), Penicillin/Streptomycin (PS)(Gibco, Cat. No. 15140-122; Lot. No. 2321114), 100 mm² cell culture dish (SPL, Cat. No. 20100), 150 mm² cell culture dish (SPL, Cat. No. 20150), 96-well culture plate (SPL, Cat. No. 30196), PBS pH 7.4 (Gibco, Cat. No. 10010-023; Lot. No. 2085080), TrypLE™ Express (Gibco, Cat. No. 12605-010; Lot. No. 2323417), Counting Chamber (Hematocytometer)(Marienfeld Superior, Cat. No. 0650010) and 0.4% Trypan Blue Solution (DYNEBIO, Cat. No. CBT3710; Lot. No. 20211201) were used.

### (2) Treatment of Compounds of the Present Invention and Method of Luciferase Assay

The compounds of Examples were completely dissolved in DMSO (Sigma-Aldrich Cat. No. D2438, Lot. No. RNBJ9566) and used in the experiment.

In the case of HeLa LgBit (Plk1-HiBit KI), the compounds were treated after being released after thymidine block, and the process was as follows. Thymidine (Sigma-Aldrich Cat. No. T9250-5G) was completely dissolved in DW and used in the experiment. For thymidine block, the products were treated with 2 mM of thymidine and then incubated for 24 hours. For release and chemical treatment, the medium was suctioned and washed with 1×PBS. TrypLE™ was added and incubated in 37° C. CO₂ incubator for 5 min. Cells neutralized by adding complete media were counted through a counter. For each well of a 96-well culture plate (SPL), 3.3×10⁴ cells and a total medium volume of 150 μL were seeded and incubated in a CO₂ incubator.

Each cell line was incubated in a CO₂ incubator for 18 hours, and Endurazine was added to each well to make up 4% of the total volume. After adding the compound of the present invention in a 96-well white plate (SPL) to a concentration of 300 nM, the wavelength of the plate reader (BMG Labtech, CLARIOstar Plus) was set to 470-480 nM, and then the luminescence was tracked in real time. After 9 hours, the luminescence value was obtained and displayed as a bar graph through an Excel program.

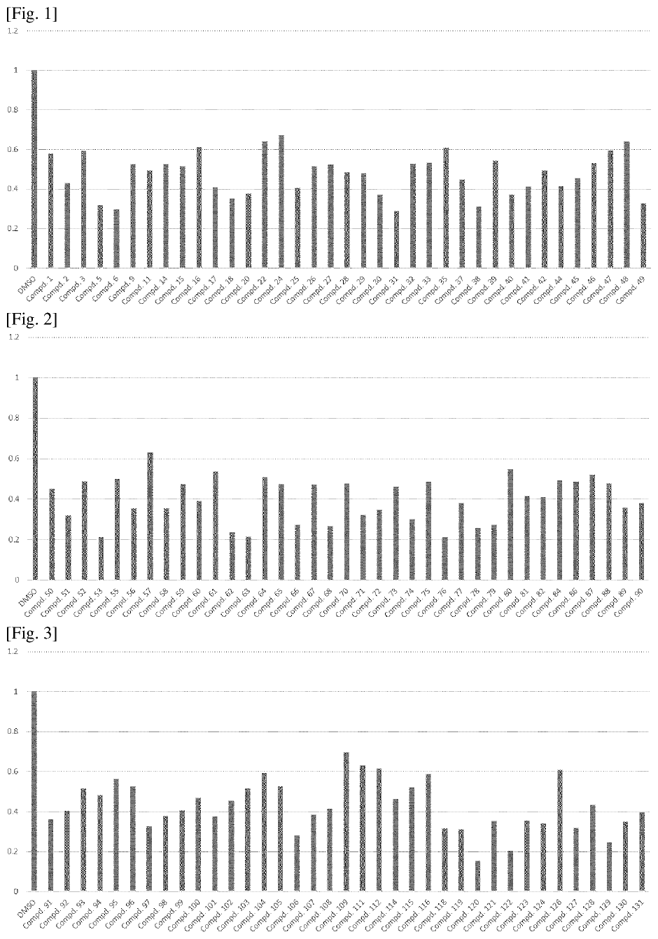

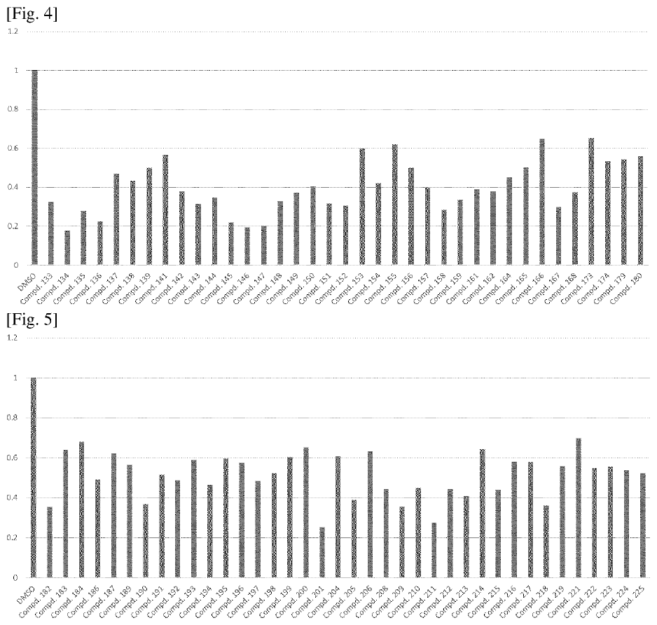

The results are shown in Table 2 below and FIG. 1 to 5.

TABLE 2

| Examplary Compound | Activity |
|---|---|
| Compound 1 | ++ |
| Compound 2 | ++ |
| Compound 3 | ++ |
| Compound 5 | ++ |
| Compound 6 | +++ |
| Compound 9 | ++ |
| Compound 11 | ++ |
| Compound 14 | ++ |
| Compound 15 | ++ |
| Compound 16 | + |
| Compound 17 | ++ |
| Compound 18 | ++ |
| Compound 20 | ++ |
| Compound 22 | + |
| Compound 24 | + |
| Compound 25 | ++ |
| Compound 26 | ++ |
| Compound 27 | ++ |
| Compound 28 | ++ |
| Compound 29 | ++ |
| Compound 30 | ++ |
| Compound 31 | +++ |
| Compound 32 | ++ |
| Compound 33 | ++ |
| Compound 35 | + |
| Compound 37 | ++ |
| Compound 38 | ++ |
| Compound 39 | ++ |
| Compound 40 | ++ |
| Compound 41 | ++ |
| Compound 42 | ++ |
| Compound 44 | ++ |
| Compound 45 | ++ |
| Compound 46 | ++ |
| Compound 47 | ++ |
| Compound 48 | + |
| Compound 49 | ++ |
| Compound 50 | ++ |
| Compound 51 | ++ |
| Compound 52 | ++ |
| Compound 53 | +++ |
| Compound 55 | ++ |
| Compound 56 | ++ |
| Compound 57 | + |
| Compound 58 | ++ |
| Compound 59 | ++ |
| Compound 60 | ++ |
| Compound 61 | ++ |
| Compound 62 | +++ |
| Compound 63 | +++ |
| Compound 64 | ++ |
| Compound 65 | ++ |
| Compound 66 | +++ |
| Compound 67 | ++ |
| Compound 68 | +++ |
| Compound 70 | ++ |
| Compound 71 | ++ |
| Compound 72 | ++ |
| Compound 73 | ++ |
| Compound 74 | ++ |
| Compound 75 | ++ |
| Compound 76 | +++ |
| Compound 77 | ++ |
| Compound 78 | +++ |
| Compound 79 | +++ |
| Compound 80 | ++ |
| Compound 81 | ++ |
| Compound 82 | ++ |
| Compound 84 | ++ |
| Compound 86 | ++ |
| Compound 87 | ++ |
| Compound 88 | ++ |
| Compound 89 | ++ |
| Compound 90 | ++ |
| Compound 91 | ++ |
| Compound 92 | ++ |
| Compound 93 | ++ |
| Compound 94 | ++ |
| Compound 95 | ++ |
| Compound 96 | ++ |
| Compound 97 | ++ |
| Compound 98 | ++ |
| Compound 99 | ++ |
| Compound 100 | ++ |
| Compound 101 | ++ |
| Compound 102 | ++ |
| Compound 103 | ++ |
| Compound 104 | ++ |
| Compound 105 | ++ |
| Compound 106 | +++ |
| Compound 107 | ++ |
| Compound 108 | ++ |
| Compound 109 | + |
| Compound 111 | + |
| Compound 112 | + |
| Compound 114 | ++ |
| Compound 115 | ++ |
| Compound 116 | ++ |
| Compound 118 | ++ |
| Compound 119 | ++ |
| Compound 120 | +++ |
| Compound 121 | ++ |
| Compound 122 | +++ |
| Compound 123 | ++ |
| Compound 124 | ++ |
| Compound 126 | + |
| Compound 127 | ++ |
| Compound 128 | ++ |
| Compound 129 | +++ |
| Compound 130 | ++ |
| Compound 131 | ++ |
| Compound 133 | ++ |
| Compound 134 | +++ |
| Compound 135 | +++ |
| Compound 136 | +++ |
| Compound 137 | ++ |
| Compound 138 | ++ |
| Compound 139 | ++ |
| Compound 141 | ++ |
| Compound 142 | ++ |
| Compound 143 | ++ |
| Compound 144 | ++ |
| Compound 145 | +++ |
| Compound 146 | +++ |
| Compound 147 | +++ |
| Compound 148 | ++ |
| Compound 149 | ++ |
| Compound 150 | ++ |
| Compound 151 | ++ |
| Compound 152 | ++ |
| Compound 153 | ++ |
| Compound 154 | ++ |
| Compound 155 | + |
| Compound 156 | ++ |
| Compound 157 | ++ |
| Compound 158 | +++ |
| Compound 159 | ++ |
| Compound 161 | ++ |
| Compound 162 | ++ |
| Compound 164 | ++ |
| Compound 165 | ++ |
| Compound 166 | + |
| Compound 167 | +++ |
| Compound 168 | ++ |
| Compound 173 | + |
| Compound 174 | ++ |
| Compound 179 | ++ |
| Compound 180 | ++ |
| Compound 182 | ++ |
| Compound 183 | + |
| Compound 184 | + |
| Compound 186 | ++ |
| Compound 187 | + |
| Compound 189 | ++ |
| Compound 190 | ++ |
| Compound 191 | ++ |
| Compound 192 | ++ |
| Compound 193 | ++ |
| Compound 194 | ++ |
| Compound 195 | ++ |
| Compound 196 | ++ |
| Compound 197 | ++ |
| Compound 198 | ++ |
| Compound 199 | + |
| Compound 200 | + |
| Compound 201 | +++ |
| Compound 204 | + |
| Compound 205 | ++ |
| Compound 206 | + |
| Compound 208 | ++ |
| Compound 209 | ++ |
| Compound 210 | ++ |
| Compound 211 | +++ |
| Compound 212 | ++ |
| Compound 213 | ++ |
| Compound 214 | + |
| Compound 215 | ++ |
| Compound 216 | ++ |
| Compound 217 | ++ |
| Compound 218 | ++ |
| Compound 219 | ++ |
| Compound 221 | + |
| Compound 222 | ++ |
| Compound 223 | ++ |
| Compound 224 | ++ |
| Compound 225 | ++ |

In Table 2, Activity represents the ratio of the luminescense value of each Exemplary Compound treatment group to DMSO treatment group (+++: <0.3, ++<0.6, +<0.7).

### 3. Cell Viability Assay

### (1) Culture of NCI-H526 Cell Line

The NCI-H526 (hereafter H526) cell line was purchased from Korea Cell Line Bank (KCLB, Seoul, Korea). For cell culture, RPMI 1640 (Gibco, Cat. No. 22400-089; Lot. No. 2277021), FBS (Gibco, Cat. No. 16000-044; Lot. No. 2351176P), Penicillin/Streptomycin (PS)(Gibco, Cat. No. 15140-122; Lot. No. 2321114), 75T cell culture flask (SPL, Cat. No. 71075), 175T cell culture flask (SPL, Cat. No. 71175), 96-well cell culture plate (SPL, Cat. No. 30096), PBS pH 7.4 (Gibco, Cat. No. 10010-023; Lot. No. 2085080), TrypLE™ Express (Gibco, Cat. No. 12605-010; Lot. No. 2323417), Counting Chamber (Hematocytometer)(Marienfeld Superior, Cat. No. 0650010), and 0.4% Trypan Blue Solution (DYNEBIO, Cat. No. CBT3710; Lot. No. 20211201) were used.

### (2) Treatment of Compounds of the Present Invention and Method of Cell Viability Assay

The compounds of Examples were completely dissolved in DMSO (Sigma-Aldrich Cat. No. D2438, Lot. No. RNBJ9566) and used in the experiment.

3×10⁴ cells were seeded for each well of a 96-well plate (SPL), and the cells were cultured in total volume of 150 μL.

Each compound was diluted 3-folds from the highest concentration of 3000 nM to the lowest concentration of 0.46 nM. After treating the compound to each well to make the total volume of 200 μL, it was cultured in a CO₂ incubator (Thermo Fisher Science, Cat. No. 4111) for 5 days.

Then, after treating EZ-Cytox (DOGEN, Cat. NO. EZ-3000, Lot. No. DLS2109) 20 μL in each well, it was cultured in CO₂ incubator for 4 hours. The absorbance of the completely cultured sample was measured by setting the wavelength of a plate reader (BMG Labtech, CLARIOstar Plus) to 450 nM, and was measured after shaking for 3 minutes in a plate reader before measurement. The final measured value was arranged with Excel program, a graph was displayed through Prism-GraphPad program, and the IC₅₀ value was measured.

The results are shown in Table 3 below.

TABLE 3
Cell Viability Assay for H526 cell

| Examplary Compound | Activity |
|---|---|
| Compound 2 | D |
| Compound 5 | E |
| Compound 6 | E |
| Compound 15 | B |
| Compound 18 | A |
| Compound 20 | D |
| Compound 25 | B |
| Compound 26 | B |
| Compound 29 | A |
| Compound 30 | C |
| Compound 31 | C |
| Compound 32 | B |
| Compound 33 | D |
| Compound 37 | E |
| Compound 38 | D |
| Compound 39 | C |
| Compound 40 | D |
| Compound 41 | D |
| Compound 42 | C |
| Compound 44 | C |
| Compound 45 | C |
| Compound 49 | B |
| Compound 50 | A |
| Compound 51 | A |
| Compound 52 | A |
| Compound 53 | B |
| Compound 55 | C |
| Compound 56 | A |
| Compound 58 | B |
| Compound 59 | A |
| Compound 60 | A |
| Compound 62 | C |
| Compound 63 | B |
| Compound 66 | B |
| Compound 68 | C |
| Compound 71 | B |
| Compound 72 | C |
| Compound 74 | D |
| Compound 76 | C |
| Compound 77 | C |
| Compound 78 | C |
| Compound 79 | C |
| Compound 81 | B |
| Compound 82 | A |
| Compound 86 | E |
| Compound 89 | E |
| Compound 90 | E |
| Compound 91 | E |
| Compound 92 | D |
| Compound 94 | E |
| Compound 97 | E |
| Compound 98 | E |
| Compound 99 | E |
| Compound 102 | E |
| Compound 103 | D |
| Compound 106 | C |
| Compound 107 | C |
| Compound 108 | C |
| Compound 114 | D |
| Compound 115 | E |
| Compound 118 | B |
| Compound 119 | D |
| Compound 120 | B |
| Compound 121 | D |
| Compound 122 | A |
| Compound 123 | C |
| Compound 124 | A |
| Compound 127 | C |
| Compound 128 | B |
| Compound 129 | A |
| Compound 130 | A |
| Compound 131 | B |
| Compound 133 | B |
| Compound 134 | A |
| Compound 135 | B |
| Compound 136 | A |
| Compound 137 | A |
| Compound 138 | B |
| Compound 139 | B |
| Compound 142 | C |
| Compound 143 | C |
| Compound 144 | B |
| Compound 145 | B |
| Compound 146 | B |
| Compound 147 | B |
| Compound 148 | B |
| Compound 149 | B |
| Compound 150 | B |
| Compound 151 | B |
| Compound 152 | B |
| Compound 153 | B |
| Compound 154 | C |
| Compound 155 | C |
| Compound 156 | B |
| Compound 157 | C |
| Compound 158 | B |
| Compound 159 | A |
| Compound 161 | A |
| Compound 162 | C |
| Compound 164 | A |
| Compound 165 | A |
| Compound 167 | A |
| Compound 190 | A |
| Compound 194 | B |
| Compound 197 | B |
| Compound 201 | B |
| Compound 205 | B |
| Compound 208 | C |
| Compound 209 | B |
| Compound 210 | B |
| Compound 211 | A |
| Compound 212 | A |
| Compound 213 | A |
| Compound 215 | E |
| Compound 216 | D |
| Compound 217 | E |
| Compound 218 | B |
| Compound 219 | A |

In Table 3, Activity represents IC₅₀ value of each Exemplary Compound treatment group to H526 cell line (A: <30 nM, B: <50 nM, C: <100 nM, D: <200 nM, E: <400 nM).

### 4. Cell Viability Assay for MRC-5 Cell Line

### (1) Culture of MRC-5 Cell Line

The MRC-5 cell line was purchased from Korea Cell Line Bank (KCLB), Seoul, Korea. Passage of cultured cells was maintained within P15.

For cell culture, MEM/EBSS (Hyclone, Cat. No. SH30024.01; Lot. No. AG29697698), FBS (Gibco, Cat. No. 16000-044; Lot. No. 2234018P), Penicillin/Streptomycin (PS)(Gibco, Cat. No. 15140-122; Lot. No. 2211099), 175T cell culture flask (SPL, Cat. No. 71175), 96-well cell culture plate (SPL, Cat. No. 30096), PBS pH 7.4 (Gibco, Cat. No. 10010-023; Lot. No. 2085080), TrypLE™ Express (Gibco, Cat. No. 12605-010; Lot. No. 2070638), Counting Chamber (Hematocytometer)(Hirschmann, Cat. No. 8100204), and 0.4% Trypan Blue Solution (DYNEBIO, Cat. No. CBT3710; Lot. No. 20190723) were used.

### (2) Treatment of Compounds of the Present Invention

MRC-5 cell line cultured in 175T cell culture flask was isolated using TrypLE™ Express. 6×10³ cells were seeded for each well of a 96-well plate (SPL), and the cells were cultured in total volume of 150 μL.

The compounds of Examples were completely dissolved in DMSO (Sigma-Aldrich, Cat. No. D2438-50ML, Lot. No. RNBK6387) and used in the experiment. Each compound was diluted 3-folds from the highest concentration of 10000 nM to the lowest concentration of 1.52 nM. Each well was mixed with a medium and treated, and the volume was set to 50 μL, so that the total volume of each well was 200 μL. Then, it was cultured in 37° C. CO₂ incubator (Thermo Fisher Science, Cat. No. 4111, Lot. No. 300512709) for 5 days.

The following compounds were used as comparative examples, and the cell viability assay was performed in the same manner as in the compounds of Examples.

### Comparative Example 1. Exemplary Compound Described in Mu et al. BBRC, 2020, 521(4): 833 (Comparative Compound 1)

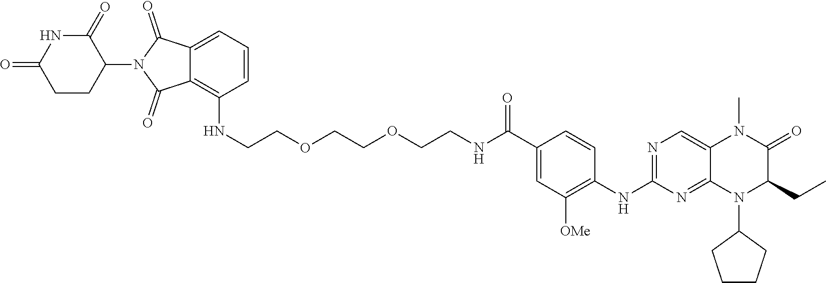 [SMILES: CC[C@@H]1C(=O)N(C)c2cnc(Nc3ccc(C(=O)NCCOCCOCCNc4cccc5c4C(=O)N(C4CCC(=O)NC4=O)C5=O)cc3OC)nc2N1C1CCCC1]

### Comparative Example 2. BT2536 (Comparative Compound 2)

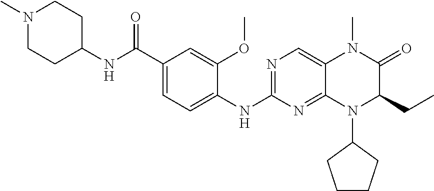 [SMILES: CC[C@@H]1C(=O)N(C)c2cnc(Nc3ccc(C(=O)NC4CCN(C)CC4)cc3OC)nc2N1C1CCCC1]

### Comparative Example 3. Volasertib (Comparative Compound 3)

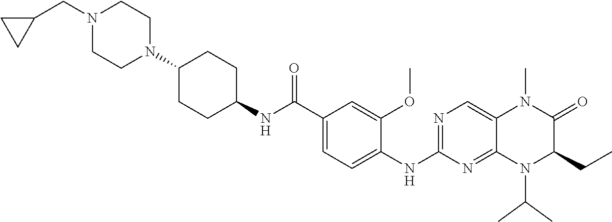 [SMILES: CC[C@@H]1C(=O)N(C)c2cnc(Nc3ccc(C(=O)N[C@H]4CC[C@H](N5CCN(CC6CC6)CC5)CC4)cc3OC)nc2N1C(C)C]

### Comparative Example 4. TAK960 (Comparative Compound 4)

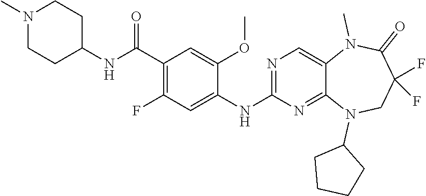 [SMILES: COc1cc(C(=O)NC2CCN(C)CC2)c(F)cc1Nc1ncc2c(n1)N(C1CCCC1)CC(F)(F)C(=O)N2C]

### (3) Cytotoxicity Experiment

After treating EZ-Cytox (DOGEN, Cat. NO. EZ-3000, Lot. No. DLS2112) 20 μL in each well of completely cultured plate, it was cultured in 37° C. CO₂ incubator for 4 hours. The 96-well plate was placed in a plate reader (BMG Labtech, Clariostar Plus), mixed for 2 minutes, and absorbance was measured at 450 nM wavelength. The data were converted into graphs using the Prism (ver.9) program.

The results are shown in Table 4 and Table 5 below.

TABLE 4
Cell Viability Assay for MRC-5 cell line

| Examplary Compound | Activity |
|---|---|
| Compound 5 | 20032 |
| Compound 9 | N.D. |
| Compound 15 | 16696 |
| Compound 18 | N.D. |
| Compound 25 | N.D. |
| Compound 26 | N.D. |
| Compound 28 | N.D. |
| Compound 30 | N.D. |
| Compound 31 | N.D. |
| Compound 32 | N.D. |
| Compound 38 | N.D. |
| Compound 39 | N.D. |
| Compound 40 | N.D. |
| Compound 41 | N.D. |
| Compound 44 | N.D. |
| Compound 45 | N.D. |
| Compound 49 | N.D. |
| Compound 50 | 11265 |
| Compound 51 | N.D. |
| Compound 55 | 3812 |
| Compound 56 | N.D. |
| Compound 58 | N.D. |
| Compound 59 | N.D. |
| Compound 60 | 4188 |
| Compound 62 | N.D. |
| Compound 63 | N.D. |
| Compound 66 | N.D. |
| Compound 68 | N.D. |
| Compound 71 | N.D. |
| Compound 72 | N.D. |
| Compound 74 | N.D. |
| Compound 76 | N.D. |
| Compound 77 | N.D. |
| Compound 78 | N.D. |
| Compound 79 | N.D. |
| Compound 81 | N.D. |
| Compound 82 | N.D. |
| Compound 89 | N.D. |
| Compound 90 | N.D. |
| Compound 91 | N.D. |
| Compound 92 | N.D. |
| Compound 98 | N.D. |
| Compound 102 | 14419 |
| Compound 106 | N.D. |
| Compound 107 | N.D. |
| Compound 108 | N.D. |
| Compound 114 | N.D. |
| Compound 115 | N.D. |
| Compound 118 | N.D. |
| Compound 119 | N.D. |
| Compound 120 | N.D. |
| Compound 121 | N.D. |
| Compound 123 | N.D. |
| Compound 124 | N.D. |
| Compound 128 | 18754 |
| Compound 129 | 17331 |
| Compound 130 | N.D. |
| Compound 131 | N.D. |
| Compound 133 | N.D. |
| Compound 136 | 6519 |
| Compound 137 | 16911 |
| Compound 138 | N.D. |
| Compound 139 | N.D. |
| Compound 142 | N.D. |
| Compound 143 | N.D. |
| Compound 144 | 4077 |
| Compound 148 | 13226 |
| Compound 149 | N.D. |
| Compound 150 | 19285 |
| Compound 151 | N.D. |
| Compound 152 | 6171 |
| Compound 153 | 15824 |
| Compound 154 | N.D. |
| Compound 155 | N.D. |
| Compound 156 | N.D. |
| Compound 157 | N.D. |
| Compound 158 | 39332 |
| Compound 159 | 4905 |
| Compound 161 | N.D. |
| Compound 162 | N.D. |
| Compound 164 | N.D. |
| Compound 165 | N.D. |
| Compound 167 | N.D. |
| Compound 182 | 9751 |
| Compound 194 | N.D. |
| Compound 197 | 18032 |
| Compound 201 | N.D. |
| Compound 205 | N.D. |
| Compound 208 | N.D. |
| Compound 209 | 5310 |
| Compound 210 | 18151 |
| Compound 211 | N.D. |
| Compound 212 | N.D. |
| Compound 213 | N.D. |
| Compound 215 | 16157 |
| Compound 216 | 27002 |
| Compound 217 | 16410 |
| Compound 219 | 12980 |

In Table 4, Activity represents IC₅₀ value (nM) of each Exemplary Compound treatment group to MRC-5 cell line. N.D. (not determined) means that cytotoxicity did not appear until 10 μM. As a result, it was confirmed that all of the compounds of the present invention specifically exhibited a high level of cytotoxicity in cancer cell lines rather than normal cell lines.

TABLE 5
Cell Viability Assay for MRC-5 cell line

| Comparative Compound | Activity |
|---|---|
| Comparative Compound 1 | 106.6 |
| Comparative Compound 2 | 3085.4 |
| Comparative Compound 3 | 2939.3 |
| Comparative Compound 4 | 9152.5 |

In Table 5, Activity represents IC₅₀ value (nM) of each Comparative Compound treatment group to MRC-5 cell line. In particular, it was found that Comparative Compound 1, a known PROTAC compound, exhibited a high level of cytotoxicity in normal cell line, unlike the Exemplary Compounds of the present invention.

1. A compound represented by the following Formula I, a stereoisomer thereof or a pharmaceutically acceptable salt thereof:
ULM-Linker-PTM [Formula 1]
in the Formula I above,
ULM is a moiety represented by the following Formula 1; 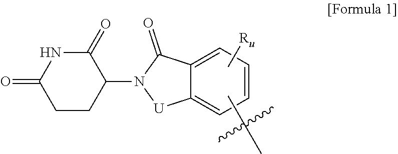
PTM is a moiety represented by the following Formula 2; 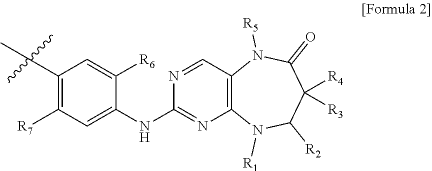
Linker is a group that chemically links ULM and PTM;
U is —CH₂— or —C(═O)—;
R_{U} is —H or -halo;
R₁ is —C₁₋₄alkyl or 3- to 7-membered cycloalkyl;
R₂ is —H;
R₃ and R₄ are each independently —H, —C₁₋₄alkyl, —C₁₋₄alkenyl or -halo, or R₃ and R₄ are linked each other to form a 3- to 6-membered ring;
R₅ is —C₁₋₄alkyl;
R₆ is —C₁₋₄alkyl, —C₁₋₄haloalkyl, —O—R_{P} or -halo;
R₇ is —H, —C₁₋₄alkyl, —C₁₋₄haloalkyl, -halo or 5- to 6-membered heterocycloalkyl {wherein at least one H of the 5- to 6-membered heterocycloalkyl ring may be substituted —C₁₋₄alkyl}, or is linked with the Linker to form a 5- to 6-membered ring; and
R_{P} is —H, —C₁₋₄alkyl, —C₁₋₄hydroxyalkyl or —C₁₋₄haloalkyl.

2. The compound, the stereoisomer thereof or the pharmaceutically acceptable salt thereof according to claim 1,
ULM is a moiety represented by following Formula 1-1 or Formula 1-2; 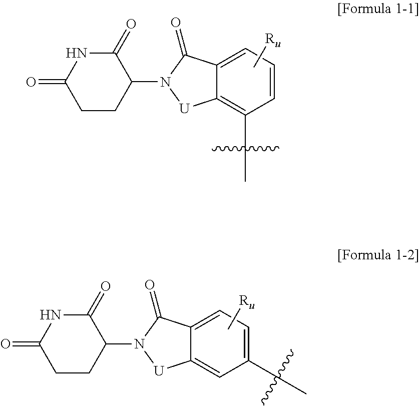
U is —CH₂— or —C(═O)—; and
R_{U} is —H or -halo.

3. The compound, the stereoisomer thereof or the pharmaceutically acceptable salt thereof according to claim 1,
ULM is 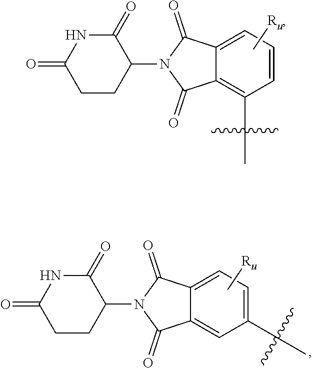 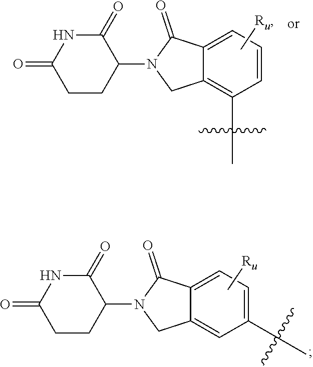
and
R_{U} is —H or -halo.

4. The compound, the stereoisomer thereof or the pharmaceutically acceptable salt thereof according to claim 1,
PTM is 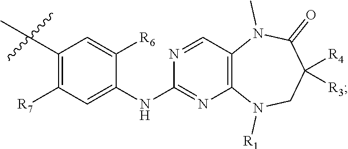
R₁ is -isopropyl, -cyclopropyl, -cyclobutyl or -cyclopentyl;
R₃ and R₄ are each independently —H, —C₁₋₄alkyl, —C₁₋₄alkenyl or -halo, or R₃ and R₄ are linked each other to form a 3-membered ring;
R₆ is —C₁₋₄alkyl, —C₁₋₄haloalkyl, —O—R_{P} or -halo;
R₇ is —H, —C₁₋₄haloalkyl, -halo or 5- to 6-membered heterocycloalkyl {wherein at least one H of the 5- to 6-membered heterocycloalkyl may be substituted —C₁₋₄alkyl}, or is linked with the Linker to form a 5- to 6-membered heterocycloalkyl ring; and
R_{P} is —H, —C₁₋₄alkyl, —C₁₋₄hydroxyalkyl or —C₁₋₄haloalkyl.

5. The compound, the stereoisomer thereof or the pharmaceutically acceptable salt thereof according to claim 1,
Linker is -L_{U}-L₁-L₂-L₃-L_{P}-;
L_{U} is —(CH₂)x-, —C≡C—, —NH—, —O—, —S— or nothing (null) {wherein L_{U} is linked with ULM [wherein, when the L_{U} is nothing (null), L₁ is directly linked with ULM], and the x is 0, 1, 2, 3 or 4};
L₁ is heterocycloalkyl or nothing (null) {wherein, when the L₁ is nothing (null), L_{U} and L₂ are directly linked, the heterocycloalkyl contains at least one N atom in the ring, and at least one H of the heterocycloalkyl ring may be substituted with —C₁₋₄alkyl, —C₁₋₄haloalkyl, —C₁₋₄alkoxy, —OH, -halo or =0};
L₂ is —(CH₂)y₁-, —(CH₂)y₂-C(═O)—(CH₂)y₃-, —C(═O)—(CH₂)y₁-C(═O)—, —(CH₂)y₂-O—(CH₂)y₃-C(═O)—, —(CH₂)y₂-NH—(CH₂)y₃-, —(CH₂)y₂-N(C₁₋₄alkyl)-(CH₂)y₃-, —(CH₂)y₂-O—(CH₂)y₃-, —(CH₂)y₁-(O—C₁₋₄alkyl)z- O—C₁₋₄alkyl-, —(CH₂)y₂-C₁₋₄alkenyl-(CH₂)y₃- or —(CH₂)y₂-phenyl-(CH₂)y₃- {wherein the y₁ to y₃ are each independently 0, 1, 2, 3, 4, 5 or 6, and the z is 1, 2, 3, 4, 5 or 6};
L₃ is cycloalkyl, heterocycloalkyl, phenyl or nothing (null) {wherein, when the L₃ is nothing (null), L₂ and L_{P} are directly linked, the heterocycloalkyl contains at least one N atom in the ring, and at least one H of the cycloalkyl, heterocycloalkyl or phenyl ring may be substituted with —C₁₋₄alkyl, —C₁₋₄haloalkyl or -halo};
L_{P} is —(CH₂)z-NR_{L}—C(═O)—, -cycloalkyl-NH—C(═O)—, -heterocycloalkyl-NH—C(═O)—, —C(═O)—, —(CH₂)z-O— or nothing (null) {wherein —(C═O)— or —O— of the L_{P} is linked with PTM [wherein, when the L_{P} is nothing (null), cycloalkyl or heterocycloalkyl of L₃ is directly linked with PTM], NR_{L} of the —(CH₂)z-NR_{L}—C(═O)— is linked with R₇ to form a 5- to 6-membered ring, and the z is 0, 1, 2, 3 or 4}; and
R_{L} is —H or —C₁₋₄alkyl.

6. The compound, the stereoisomer thereof or the pharmaceutically acceptable salt thereof according to claim 5,
L_{U} is —(CH₂)x-, —C≡C—, —NH—, —O—, —S— or nothing (null) {wherein L_{U} is linked with ULM [wherein, when the L_{U} is nothing (null), L₁ is directly linked with ULM], and the x is 0 or 1};
L₁ is 4- to 12-membered heterocycloalkyl or nothing (null) {wherein, when the L₁ is nothing (null), L_{U} and L₂ are directly linked, the 4- to 12-membered heterocycloalkyl is single ring, bridged bicyclic ring or spiro ring, the 4- to 12-membered heterocycloalkyl contains at least one N atom in the ring, and at least one H of the 4- to 12-membered heterocycloalkyl ring may be substituted with —OH or -halo};
L₂ is —(CH₂)y₁-, —(CH₂)y₂-C(═O)—(CH₂)y₃-, —C(═O)—(CH₂)y₁-C(═O)—, —(CH₂)y₂-O—(CH₂)y₃-C(═O)—, —(CH₂)y₂-N(C₁₋₄alkyl)-(CH₂)y₃-, —(CH₂)y₂-O—(CH₂)y₃-, —(CH₂)y₁-(O—C₁₋₄alkyl)z-O—C₁₋₄alkyl-, —(CH₂)y₂-C_{1- 4} alkenyl-(CH₂)y₃- or —(CH₂)y₂-phenyl-(CH₂)y₃- {wherein the y₁ to y₃ are each independently 0, 1, 2, 3, 4 or 5, and the z is 1, 2, 3, 4 or 5};
L₃ is 4- to 6-membered cycloalkyl, 4- to 12-membered heterocycloalkyl, phenyl or nothing (null) {wherein, when the L₃ is nothing (null), L₂ and L_{P} are directly linked, the 4- to 12-membered heterocycloalkyl is single ring, bridged bicyclic ring or spiro ring, the 4- to 12-membered heterocycloalkyl contains at least one N atom in the ring, and at least one H of the 4- to 6-membered cycloalkyl, 4- to 12-membered heterocycloalkyl or phenyl ring may be substituted with -halo};
L_{P} is —(CH₂)z-NR_{L}—C(═O)—, -cycloalkyl-NH—C(═O)—, -heterocycloalkyl-NH—C(═O)—, —C(═O)—, —(CH₂)z-O— or nothing (null) {wherein —(C═O)— or —O— of the L_{P} is linked with PTM [wherein, when the L_{P} is nothing (null), cycloalkyl or heterocycloalkyl of L₃ is directly linked with PTM], NR_{L} of the —(CH₂)z-NR_{L}—C(═O)— may be linked with R₇ to form a 5- to 6-membered heterocycloalkyl ring, and the z is 0 or 1}; and
R_{L} is —H or —C₁₋₄alkyl.

7. The compound, the stereoisomer thereof or the pharmaceutically acceptable salt thereof according to claim 1, wherein the compound represented by the Formula I is selected from:

| Com- | |
|---|---|
| pound | Structure |
| 1 | 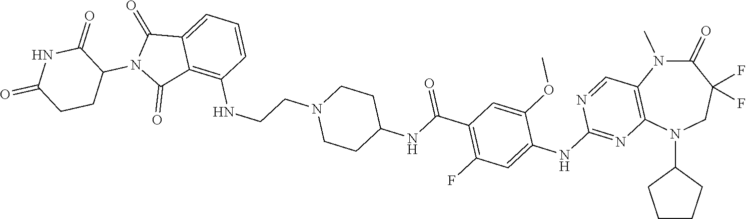 [SMILES: COc1cc(C(=O)NC2CCN(CCNc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 2 | 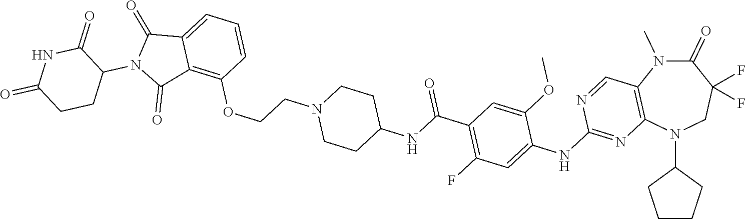 [SMILES: COc1cc(C(=O)NC2CCN(CCOc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 3 | 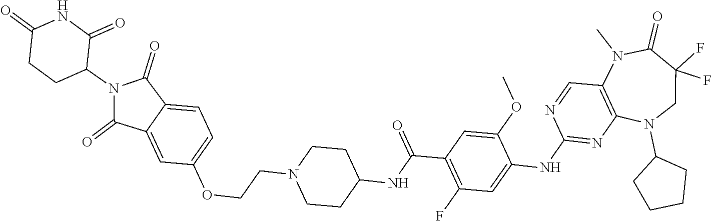 [SMILES: COc1cc(C(=O)NC2CCN(CCOc3ccc4c(c3)C(=O)N(C3CCC(=O)NC3=O)C4=O)CC2)c(F)cc1Nc1ncc2c(n1)N(C1CCCC1)CC(F)(F)C(=O)N2C] |
| 4 | 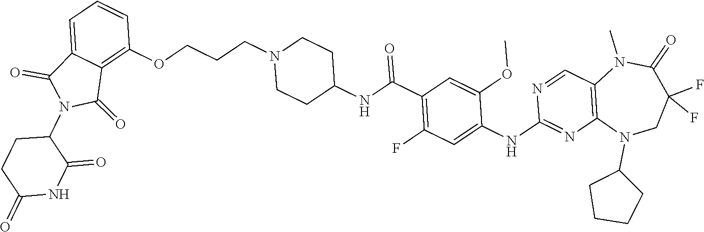 [SMILES: COc1cc(C(=O)NC2CCN(CCCOc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 5 | 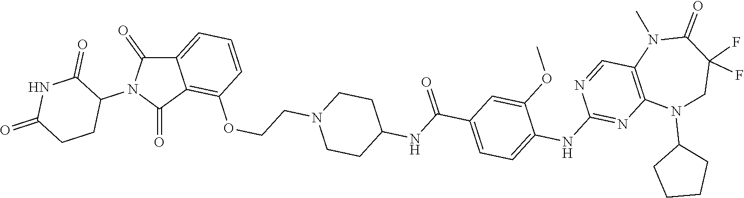 [SMILES: COc1cc(C(=O)NC2CCN(CCOc3cccc4c3C(=O)N(C3CCC(=O)NC3=O)C4=O)CC2)ccc1Nc1ncc2c(n1)N(C1CCCC1)CC(F)(F)C(=O)N2C] |
| 6 | 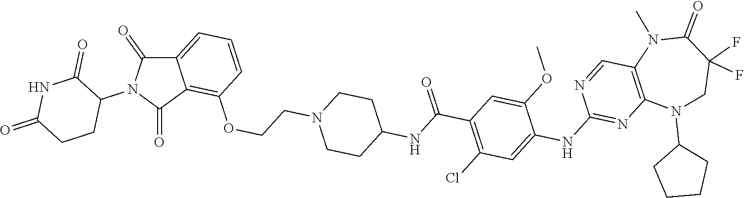 [SMILES: COc1cc(C(=O)NC2CCN(CCOc3cccc4c3C(=O)N(C3CCC(=O)NC3=O)C4=O)CC2)c(Cl)cc1Nc1ncc2c(n1)N(C1CCCC1)CC(F)(F)C(=O)N2C] |
| 7 | 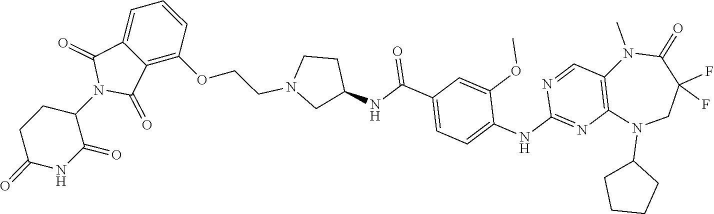 [SMILES: COc1cc(C(=O)N[C@@H]2CCN(CCOc3cccc4c3C(=O)N(C3CCC(=O)NC3=O)C4=O)C2)ccc1Nc1ncc2c(n1)N(C1CCCC1)CC(F)(F)C(=O)N2C] |
| 8 | 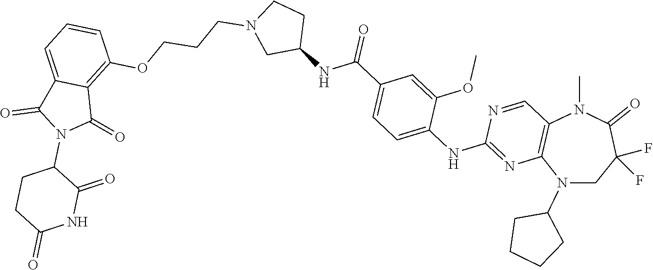 [SMILES: COc1cc(C(=O)N[C@@H]2CCN(CCCOc3cccc4c3C(=O)N(C3CCC(=O)NC3=O)C4=O)C2)ccc1Nc1ncc2c(n1)N(C1CCCC1)CC(F)(F)C(=O)N2C] |
| 9 | 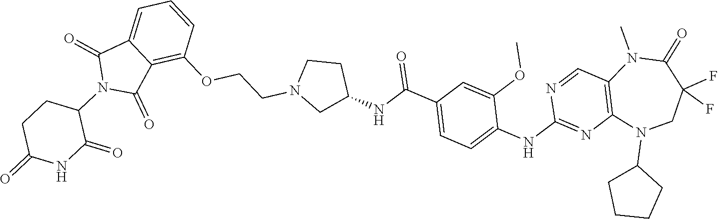 [SMILES: COc1cc(C(=O)N[C@H]2CCN(CCOc3cccc4c3C(=O)N(C3CCC(=O)NC3=O)C4=O)C2)ccc1Nc1ncc2c(n1)N(C1CCCC1)CC(F)(F)C(=O)N2C] |
| 10 | 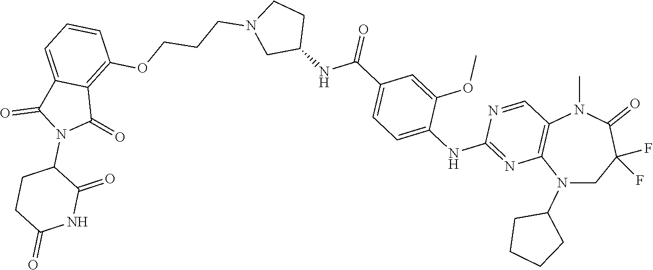 [SMILES: COc1cc(C(=O)N[C@H]2CCN(CCCOc3cccc4c3C(=O)N(C3CCC(=O)NC3=O)C4=O)C2)ccc1Nc1ncc2c(n1)N(C1CCCC1)CC(F)(F)C(=O)N2C] |
| 11 | 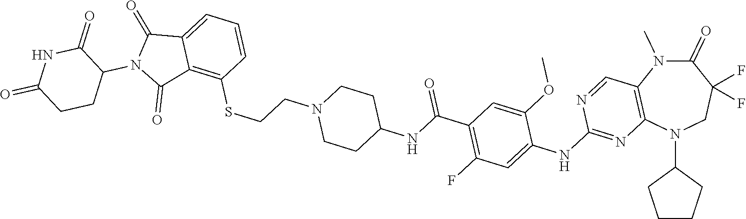 [SMILES: COc1cc(C(=O)NC2CCN(CCSc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 12 | 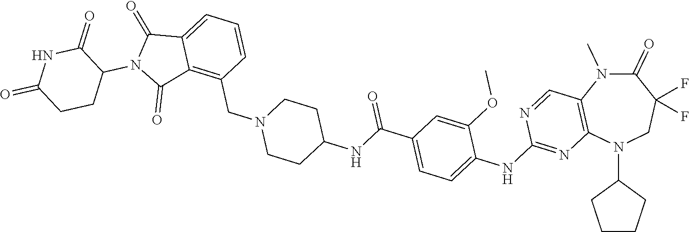 [SMILES: COc1cc(C(=O)NC2CCN(Cc3cccc4c3C(=O)N(C3CCC(=O)NC3=O)C4=O)CC2)ccc1Nc1ncc2c(n1)N(C1CCCC1)CC(F)(F)C(=O)N2C] |
| 13 | 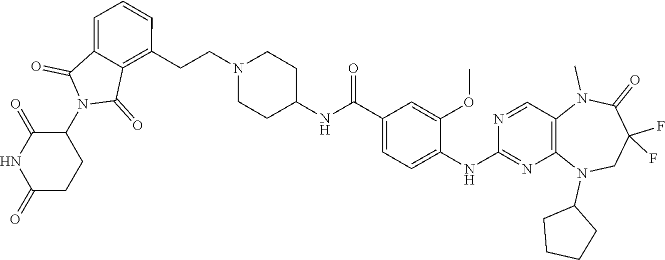 [SMILES: COc1cc(C(=O)NC2CCN(CCc3cccc4c3C(=O)N(C3CCC(=O)NC3=O)C4=O)CC2)ccc1Nc1ncc2c(n1)N(C1CCCC1)CC(F)(F)C(=O)N2C] |
| 14 | 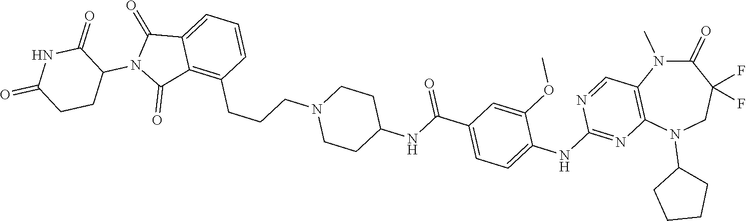 [SMILES: COc1cc(C(=O)NC2CCN(CCCc3cccc4c3C(=O)N(C3CCC(=O)NC3=O)C4=O)CC2)ccc1Nc1ncc2c(n1)N(C1CCCC1)CC(F)(F)C(=O)N2C] |
| 15 | 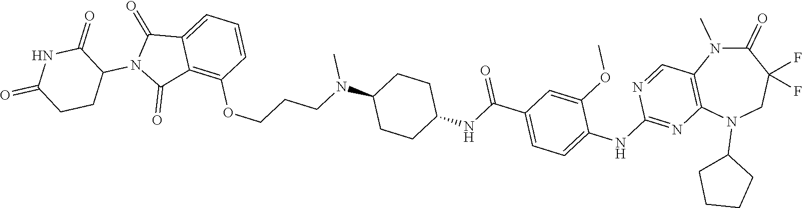 [SMILES: COc1cc(C(=O)N[C@H]2CC[C@H](N(C)CCCOc3cccc4c3C(=O)N(C3CCC(=O)NC3=O)C4=O)CC2)ccc1Nc1ncc2c(n1)N(C1CCCC1)CC(F)(F)C(=O)N2C] |
| 16 | 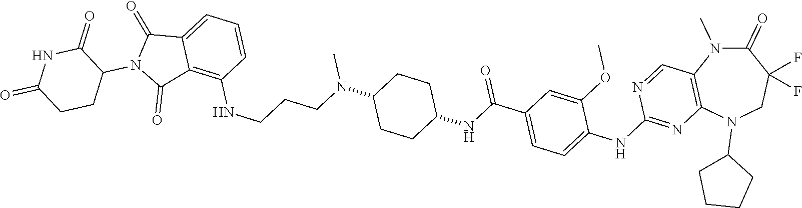 |
| 17 | 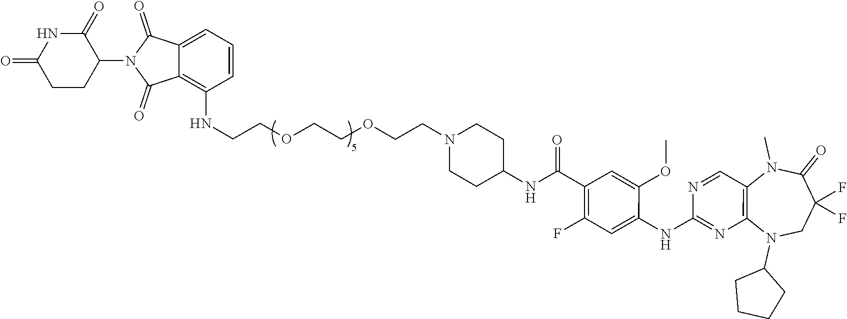 |
| 18 | 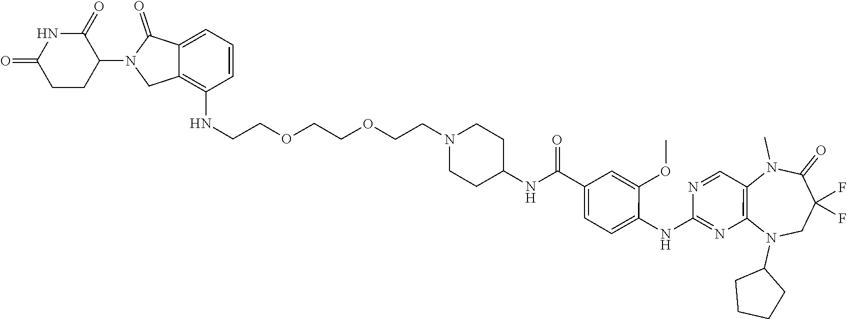 [SMILES: COc1cc(C(=O)NC2CCN(CCOCCOCCNc3cccc4c3CN(C3CCC(=O)NC3=O)C4=O)CC2)ccc1Nc1ncc2c(n1)N(C1CCCC1)CC(F)(F)C(=O)N2C] |
| 19 | 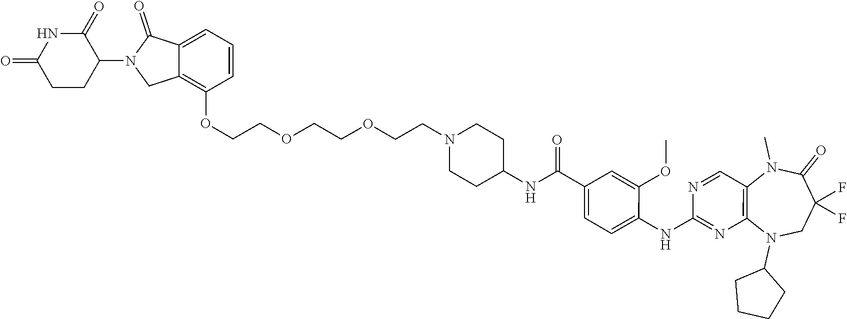 [SMILES: COc1cc(C(=O)NC2CCN(CCOCCOCCOc3cccc4c3CN(C3CCC(=O)NC3=O)C4=O)CC2)ccc1Nc1ncc2c(n1)N(C1CCCC1)CC(F)(F)C(=O)N2C] |
| 20 | 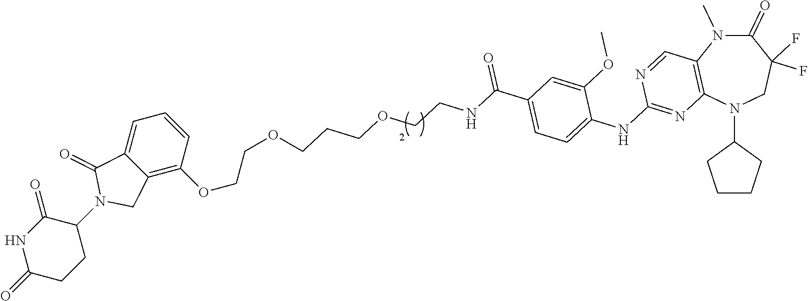 |
| 21 | 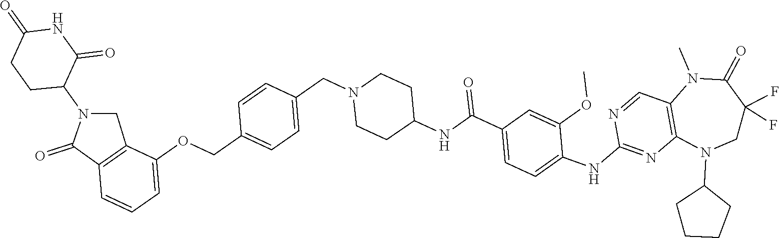 [SMILES: COc1cc(C(=O)NC2CCN(Cc3ccc(COc4cccc5c4CN(C4CCC(=O)NC4=O)C5=O)cc3)CC2)ccc1Nc1ncc2c(n1)N(C1CCCC1)CC(F)(F)C(=O)N2C] |
| 22 | 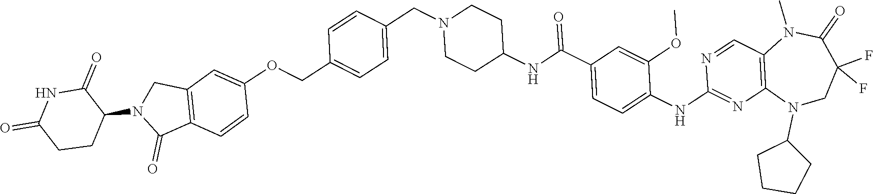 [SMILES: COc1cc(C(=O)NC2CCN(Cc3ccc(COc4ccc5c(c4)CN([C@H]4CCC(=O)NC4=O)C5=O)cc3)CC2)ccc1Nc1ncc2c(n1)N(C1CCCC1)CC(F)(F)C(=O)N2C] |
| 23 | 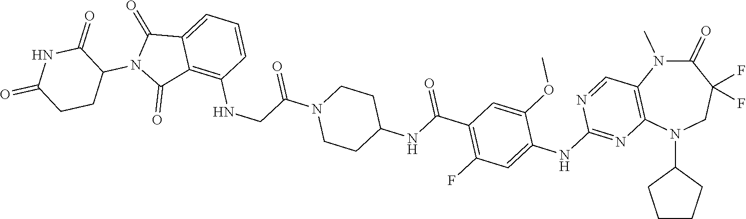 [SMILES: COc1cc(C(=O)NC2CCN(C(=O)CNc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 24 | 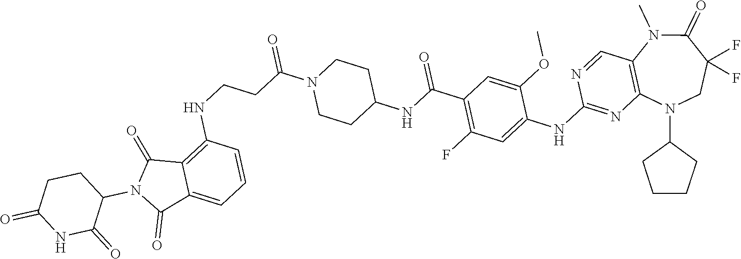 [SMILES: COc1cc(C(=O)NC2CCN(C(=O)CCNc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 25 | 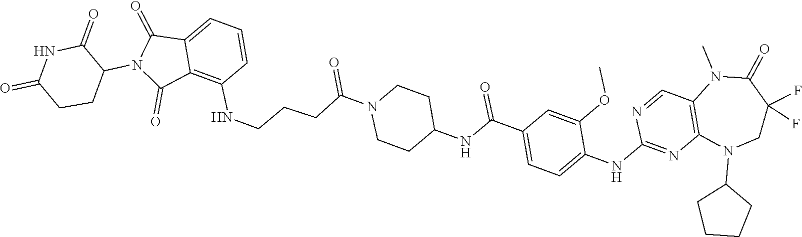 [SMILES: COc1cc(C(=O)NC2CCN(C(=O)CCCNc3cccc4c3C(=O)N(C3CCC(=O)NC3=O)C4=O)CC2)ccc1Nc1ncc2c(n1)N(C1CCCC1)CC(F)(F)C(=O)N2C] |
| 26 | 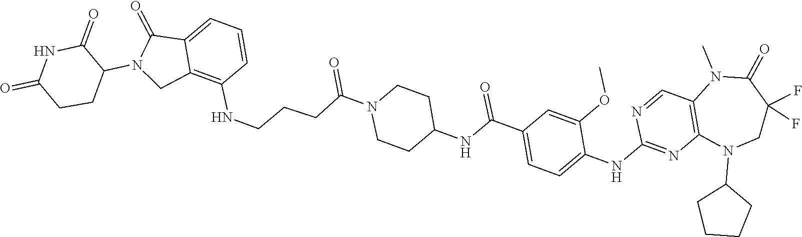 [SMILES: COc1cc(C(=O)NC2CCN(C(=O)CCCNc3cccc4c3CN(C3CCC(=O)NC3=O)C4=O)CC2)ccc1Nc1ncc2c(n1)N(C1CCCC1)CC(F)(F)C(=O)N2C] |
| 27 | 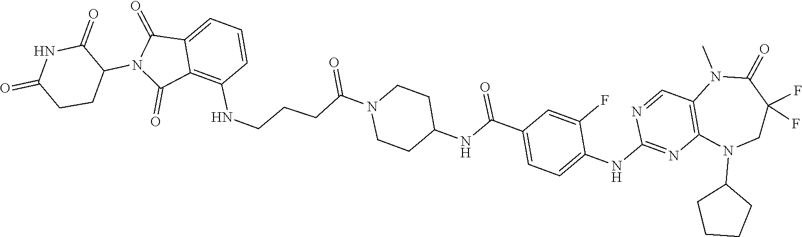 [SMILES: CN1C(=O)C(F)(F)CN(C2CCCC2)c2nc(Nc3ccc(C(=O)NC4CCN(C(=O)CCCNc5cccc6c5C(=O)N(C5CCC(=O)NC5=O)C6=O)CC4)cc3F)ncc21] |
| 28 | 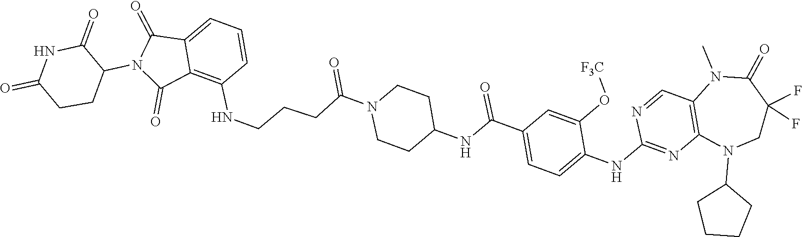 [SMILES: CN1C(=O)C(F)(F)CN(C2CCCC2)c2nc(Nc3ccc(C(=O)NC4CCN(C(=O)CCCNc5cccc6c5C(=O)N(C5CCC(=O)NC5=O)C6=O)CC4)cc3OC(F)(F)F)ncc21] |
| 29 | 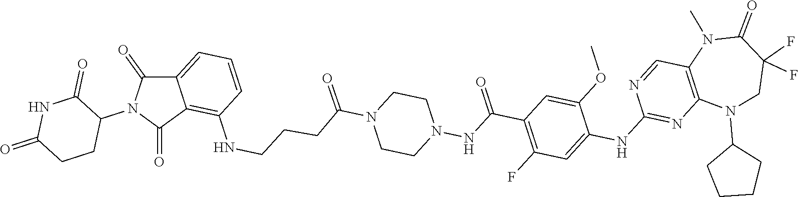 [SMILES: COc1cc(C(=O)NN2CCN(C(=O)CCCNc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 30 | 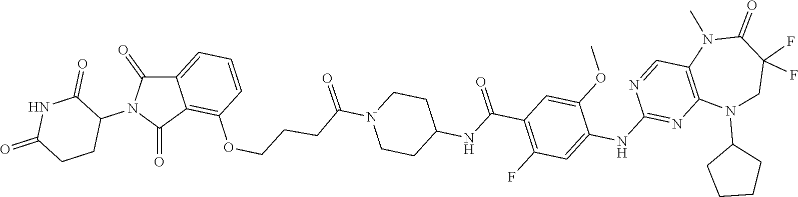 [SMILES: COc1cc(C(=O)NC2CCN(C(=O)CCCOc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 31 | 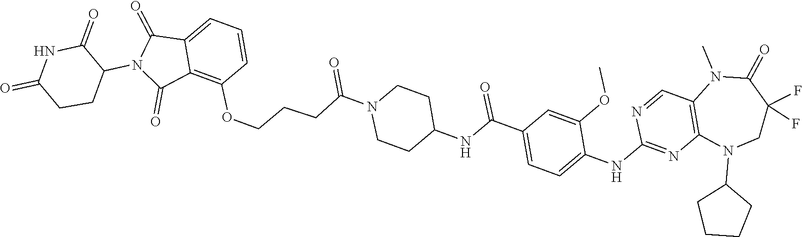 [SMILES: COc1cc(C(=O)NC2CCN(C(=O)CCCOc3cccc4c3C(=O)N(C3CCC(=O)NC3=O)C4=O)CC2)ccc1Nc1ncc2c(n1)N(C1CCCC1)CC(F)(F)C(=O)N2C] |
| 32 | 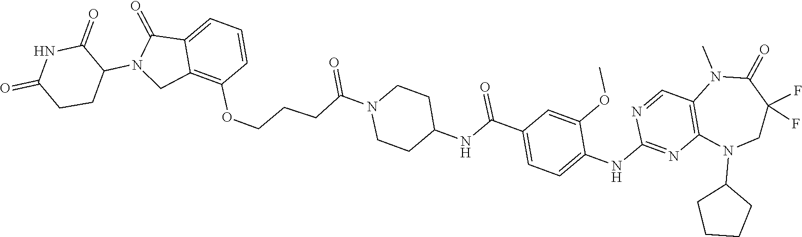 [SMILES: COc1cc(C(=O)NC2CCN(C(=O)CCCOc3cccc4c3CN(C3CCC(=O)NC3=O)C4=O)CC2)ccc1Nc1ncc2c(n1)N(C1CCCC1)CC(F)(F)C(=O)N2C] |
| 33 | 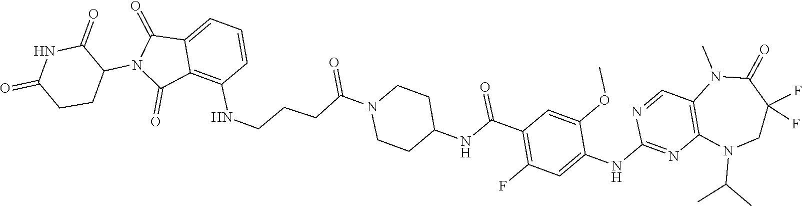 [SMILES: COc1cc(C(=O)NC2CCN(C(=O)CCCNc3cccc4c3C(=O)N(C3CCC(=O)NC3=O)C4=O)CC2)c(F)cc1Nc1ncc2c(n1)N(C(C)C)CC(F)(F)C(=O)N2C] |
| 34 | 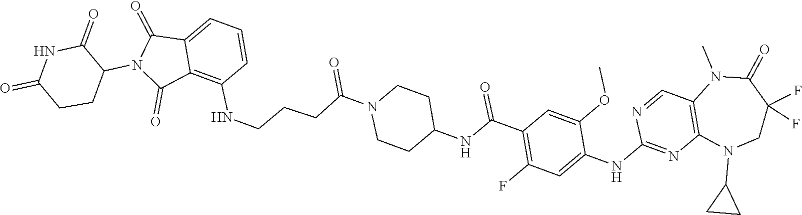 [SMILES: COc1cc(C(=O)NC2CCN(C(=O)CCCNc3cccc4c3C(=O)N(C3CCC(=O)NC3=O)C4=O)CC2)c(F)cc1Nc1ncc2c(n1)N(C1CC1)CC(F)(F)C(=O)N2C] |
| 35 | 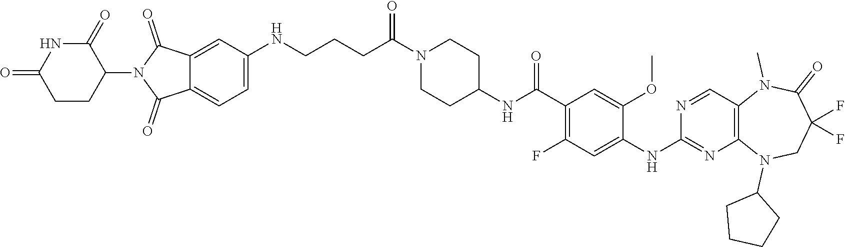 [SMILES: COc1cc(C(=O)NC2CCN(C(=O)CCCNc3ccc4c(c3)C(=O)N(C3CCC(=O)NC3=O)C4=O)CC2)c(F)cc1Nc1ncc2c(n1)N(C1CCCC1)CC(F)(F)C(=O)N2C] |
| 36 | 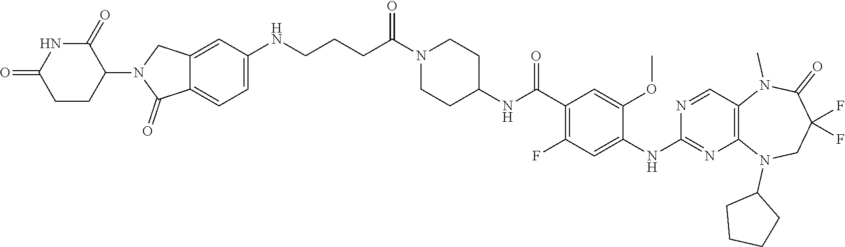 [SMILES: COc1cc(C(=O)NC2CCN(C(=O)CCCNc3ccc4c(c3)CN(C3CCC(=O)NC3=O)C4=O)CC2)c(F)cc1Nc1ncc2c(n1)N(C1CCCC1)CC(F)(F)C(=O)N2C] |
| 37 | 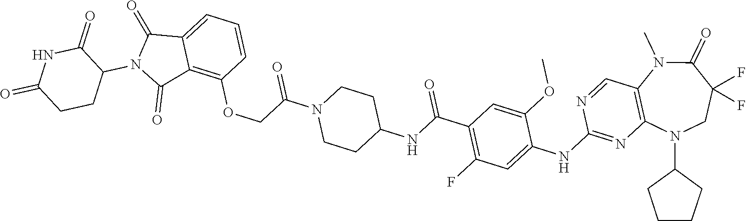 [SMILES: COc1cc(C(=O)NC2CCN(C(=O)COc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 38 | 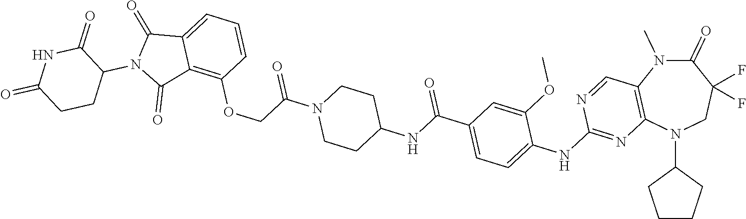 [SMILES: COc1cc(C(=O)NC2CCN(C(=O)COc3cccc4c3C(=O)N(C3CCC(=O)NC3=O)C4=O)CC2)ccc1Nc1ncc2c(n1)N(C1CCCC1)CC(F)(F)C(=O)N2C] |
| 39 | 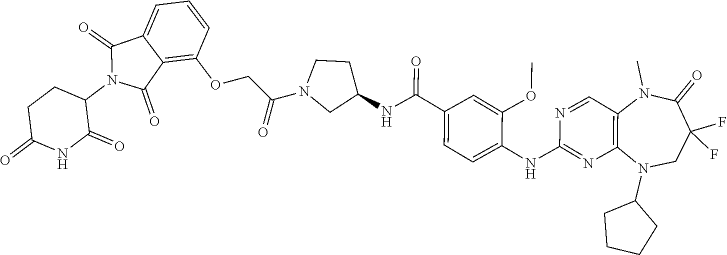 [SMILES: COc1cc(C(=O)N[C@@H]2CCN(C(=O)COc3cccc4c3C(=O)N(C3CCC(=O)NC3=O)C4=O)C2)ccc1Nc1ncc2c(n1)N(C1CCCC1)CC(F)(F)C(=O)N2C] |
| 40 | 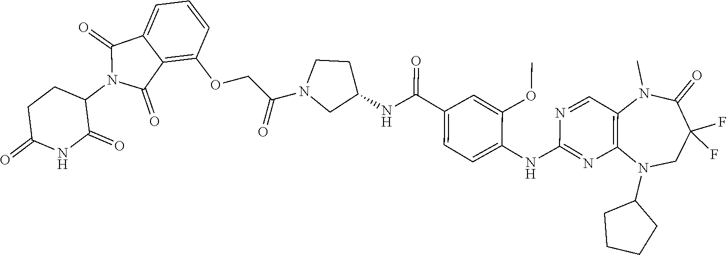 [SMILES: COc1cc(C(=O)N[C@H]2CCN(C(=O)COc3cccc4c3C(=O)N(C3CCC(=O)NC3=O)C4=O)C2)ccc1Nc1ncc2c(n1)N(C1CCCC1)CC(F)(F)C(=O)N2C] |
| 41 | 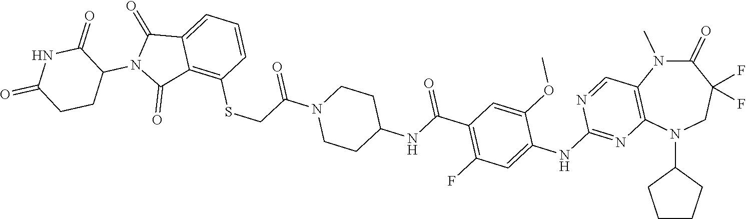 [SMILES: COc1cc(C(=O)NC2CCN(C(=O)CSc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 42 | 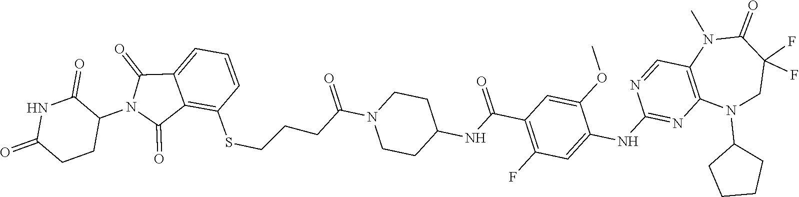 [SMILES: COc1cc(C(=O)NC2CCN(C(=O)CCCSc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 43 | 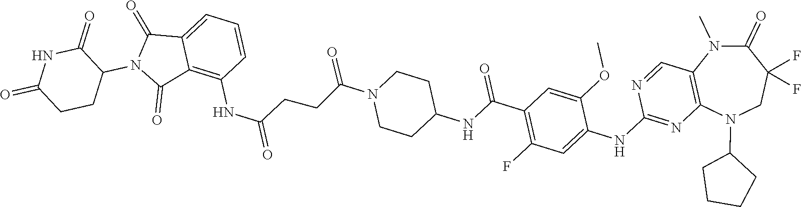 [SMILES: COc1cc(C(=O)NC2CCN(C(=O)CCC(=O)Nc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 44 | 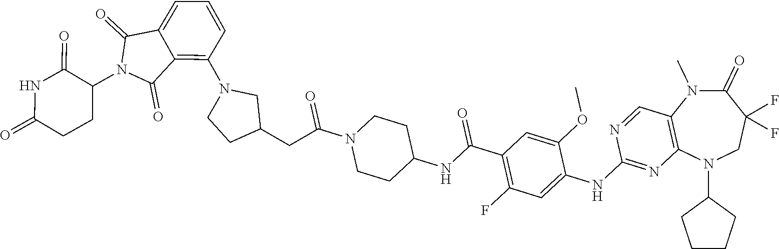 [SMILES: COc1cc(C(=O)NC2CCN(C(=O)CC3CCN(c4cccc5c4C(=O)N(C4CCC(=O)NC4=O)C5=O)C3)CC2)c(F)cc1Nc1ncc2c(n1)N(C1CCCC1)CC(F)(F)C(=O)N2C] |
| 45 | 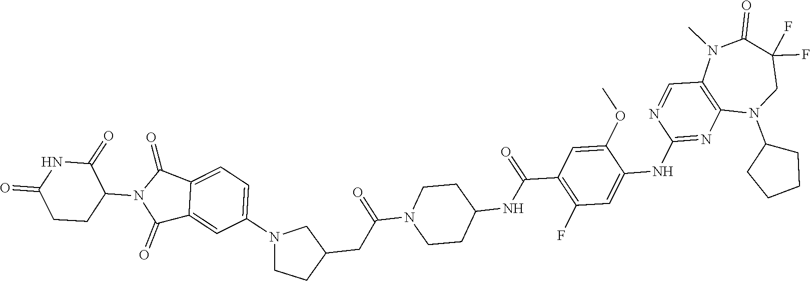 [SMILES: COc1cc(C(=O)NC2CCN(C(=O)CC3CCN(c4ccc5c(c4)C(=O)N(C4CCC(=O)NC4=O)C5=O)C3)CC2)c(F)cc1Nc1ncc2c(n1)N(C1CCCC1)CC(F)(F)C(=O)N2C] |
| 46 | 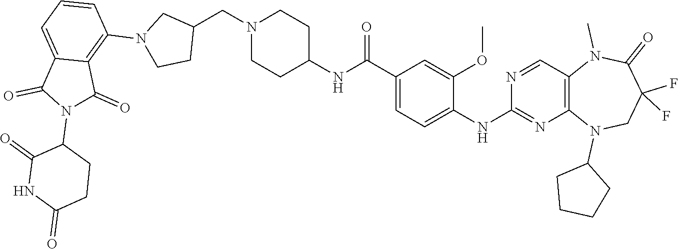 [SMILES: COc1cc(C(=O)NC2CCN(CC3CCN(c4cccc5c4C(=O)N(C4CCC(=O)NC4=O)C5=O)C3)CC2)ccc1Nc1ncc2c(n1)N(C1CCCC1)CC(F)(F)C(=O)N2C] |
| 47 | 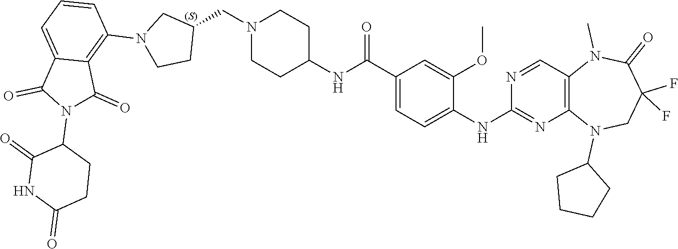 |
| 48 | 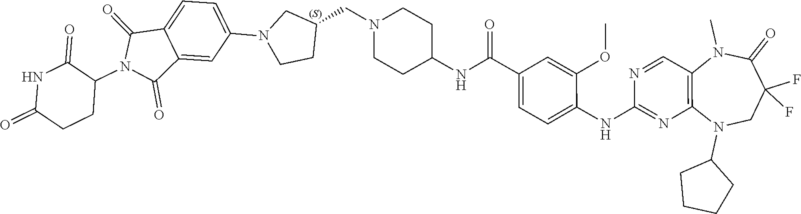 |
| 49 | 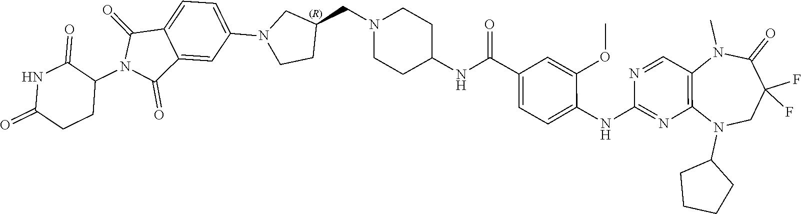 |
| 50 | 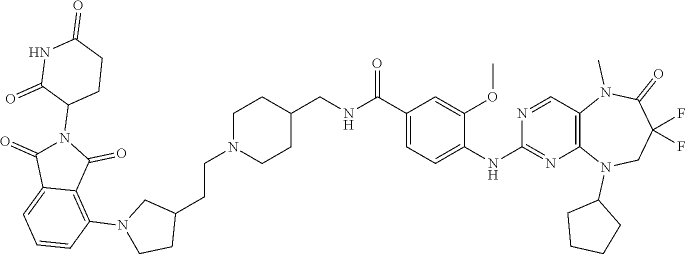 [SMILES: COc1cc(C(=O)NCC2CCN(CCC3CCN(c4cccc5c4C(=O)N(C4CCC(=O)NC4=O)C5=O)C3)CC2)ccc1Nc1ncc2c(n1)N(C1CCCC1)CC(F)(F)C(=O)N2C] |
| 51 | 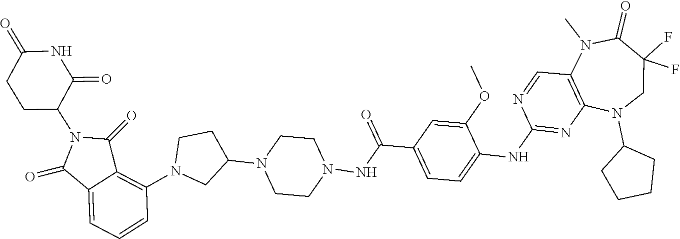 [SMILES: COc1cc(C(=O)NN2CCN(C3CCN(c4cccc5c4C(=O)N(C4CCC(=O)NC4=O)C5=O)C3)CC2)ccc1Nc1ncc2c(n1)N(C1CCCC1)CC(F)(F)C(=O)N2C] |
| 52 | 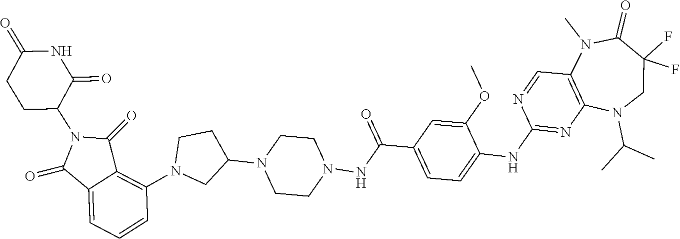 [SMILES: COc1cc(C(=O)NN2CCN(C3CCN(c4cccc5c4C(=O)N(C4CCC(=O)NC4=O)C5=O)C3)CC2)ccc1Nc1ncc2c(n1)N(C(C)C)CC(F)(F)C(=O)N2C] |
| 53 | 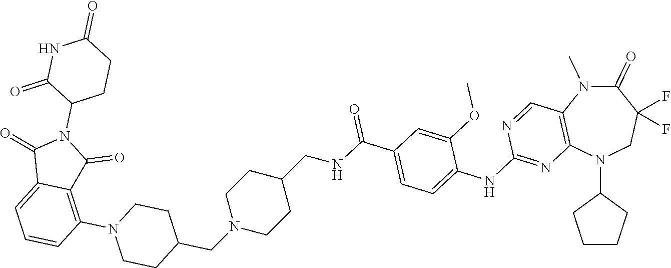 [SMILES: COc1cc(C(=O)NCC2CCN(CC3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 54 | 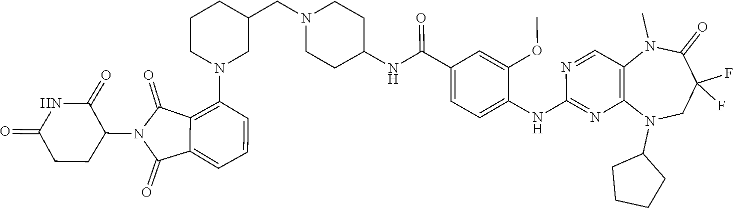 [SMILES: COc1cc(C(=O)NC2CCN(CC3CCCN(c4cccc5c4C(=O)N(C4CCC(=O)NC4=O)C5=O)C3)CC2)ccc1Nc1ncc2c(n1)N(C1CCCC1)CC(F)(F)C(=O)N2C] |
| 55 | 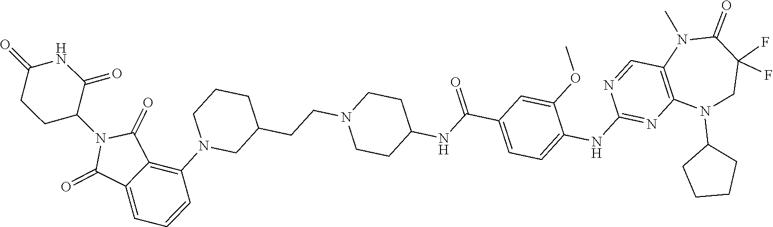 [SMILES: COc1cc(C(=O)NC2CCN(CCC3CCCN(c4cccc5c4C(=O)N(C4CCC(=O)NC4=O)C5=O)C3)CC2)ccc1Nc1ncc2c(n1)N(C1CCCC1)CC(F)(F)C(=O)N2C] |
| 56 | 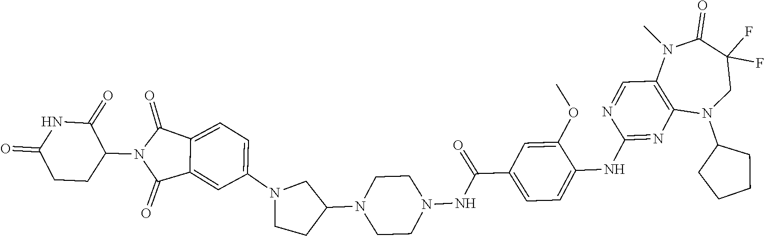 [SMILES: COc1cc(C(=O)NN2CCN(C3CCN(c4ccc5c(c4)C(=O)N(C4CCC(=O)NC4=O)C5=O)C3)CC2)ccc1Nc1ncc2c(n1)N(C1CCCC1)CC(F)(F)C(=O)N2C] |
| 57 | 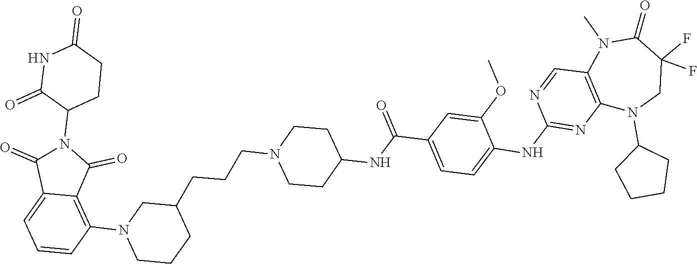 [SMILES: COc1cc(C(=O)NC2CCN(CCCC3CCCN(c4cccc5c4C(=O)N(C4CCC(=O)NC4=O)C5=O)C3)CC2)ccc1Nc1ncc2c(n1)N(C1CCCC1)CC(F)(F)C(=O)N2C] |
| 58 | 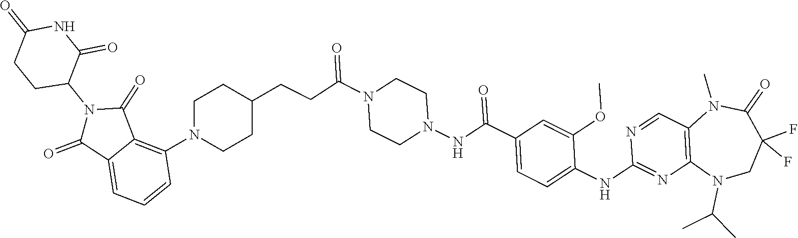 [SMILES: COc1cc(C(=O)NN2CCN(C(=O)CCC3CCN(c4cccc5c4C(=O)N(C4CCC(=O)NC4=O)C5=O)CC3)CC2)ccc1Nc1ncc2c(n1)N(C(C)C)CC(F)(F)C(=O)N2C] |
| 59 | 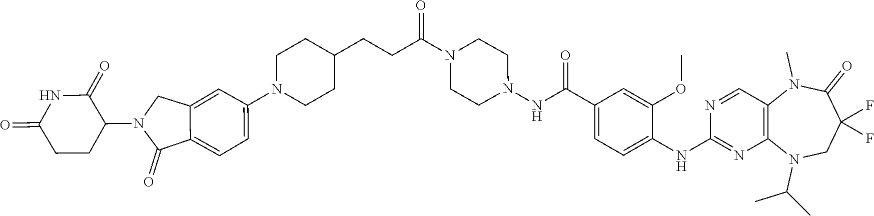 [SMILES: COc1cc(C(=O)NN2CCN(C(=O)CCC3CCN(c4ccc5c(c4)CN(C4CCC(=O)NC4=O)C5=O)CC3)CC2)ccc1Nc1ncc2c(n1)N(C(C)C)CC(F)(F)C(=O)N2C] |
| 60 | 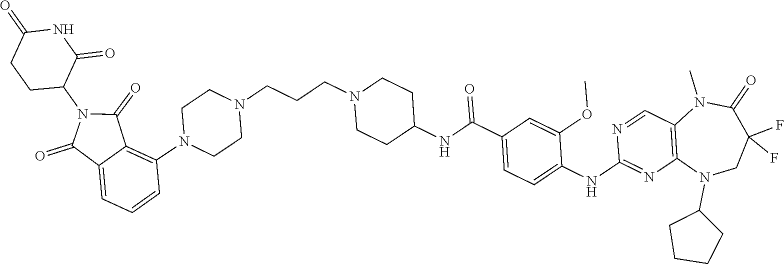 [SMILES: COc1cc(C(=O)NC2CCN(CCCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 61 | 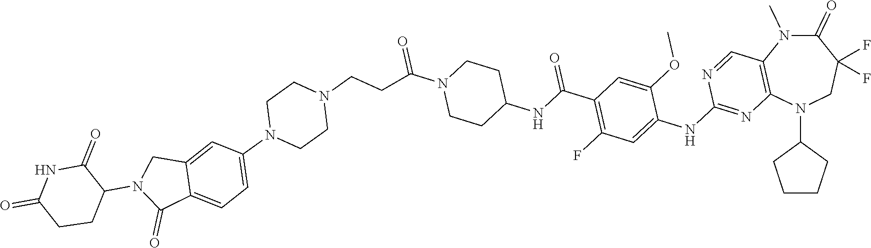 [SMILES: COc1cc(C(=O)NC2CCN(C(=O)CCN3CCN(c4ccc5c(c4)CN(C4CCC(=O)NC4=O)C5=O)CC3)CC2)c(F)cc1Nc1ncc2c(n1)N(C1CCCC1)CC(F)(F)C(=O)N2C] |
| 62 | 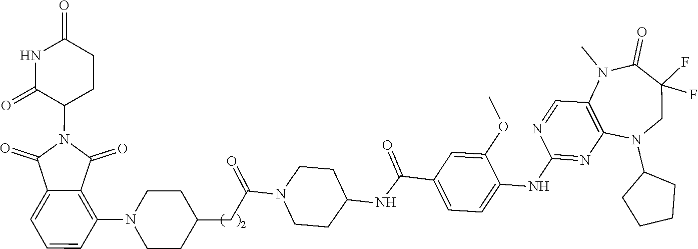 |
| 63 | 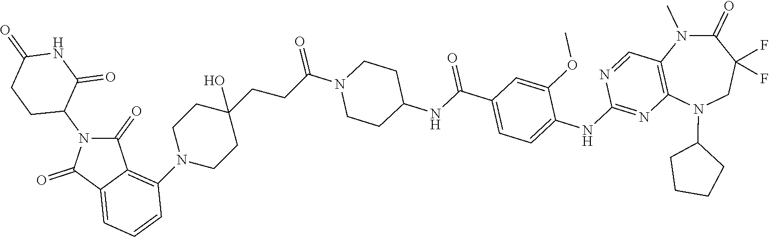 [SMILES: COc1cc(C(=O)NC2CCN(C(=O)CCC3(O)CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 64 | 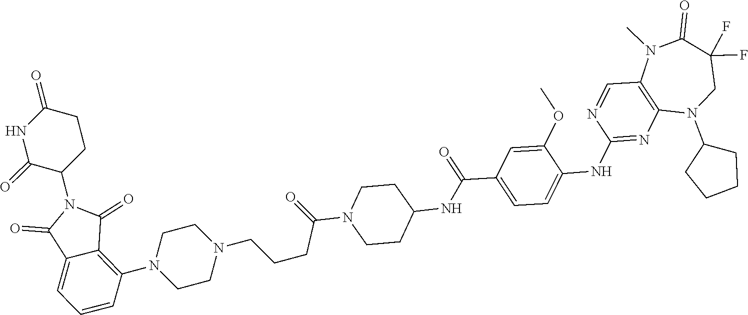 [SMILES: COc1cc(C(=O)NC2CCN(C(=O)CCCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 65 | 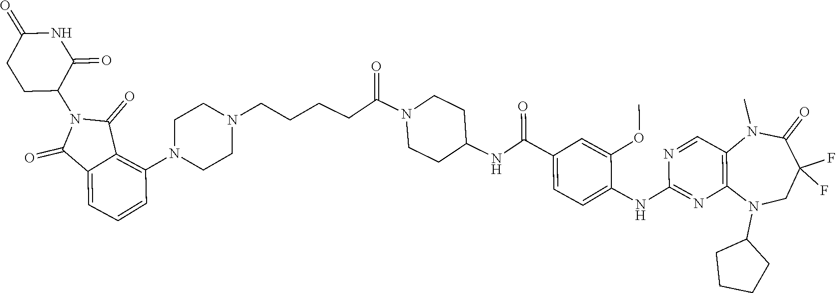 [SMILES: COc1cc(C(=O)NC2CCN(C(=O)CCCCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 66 | 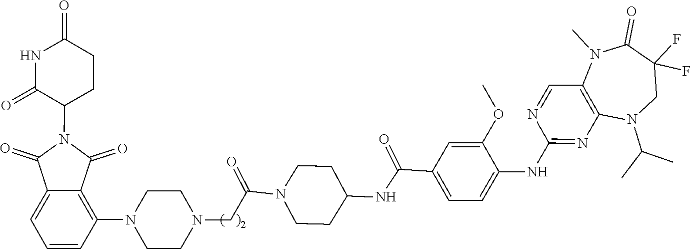 |
| 67 | 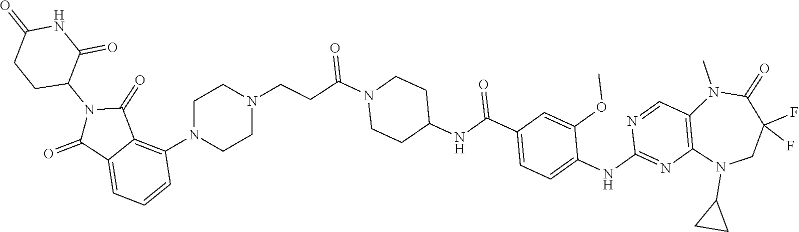 [SMILES: COc1cc(C(=O)NC2CCN(C(=O)CCN3CCN(c4cccc5c4C(=O)N(C4CCC(=O)NC4=O)C5=O)CC3)CC2)ccc1Nc1ncc2c(n1)N(C1CC1)CC(F)(F)C(=O)N2C] |
| 68 | 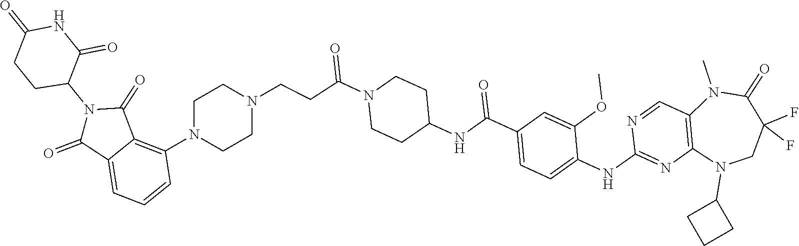 [SMILES: COc1cc(C(=O)NC2CCN(C(=O)CCN3CCN(c4cccc5c4C(=O)N(C4CCC(=O)NC4=O)C5=O)CC3)CC2)ccc1Nc1ncc2c(n1)N(C1CCC1)CC(F)(F)C(=O)N2C] |
| 69 | 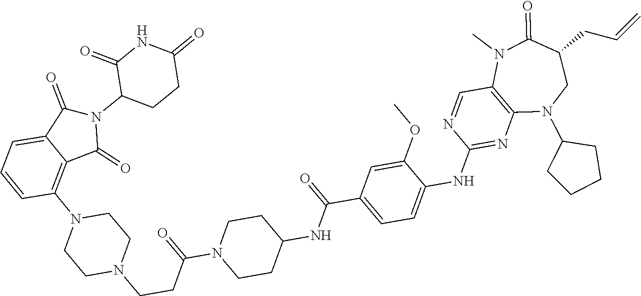 [SMILES: C=CC[C@@H]1CN(C2CCCC2)c2nc(Nc3ccc(C(=O)NC4CCN(C(=O)CCN5CCN(c6cccc7c6C(=O)N(C6CCC(=O)NC6=O)C7=O)CC5)CC4)cc3OC)ncc2N(C)C1=O] |
| 70 | 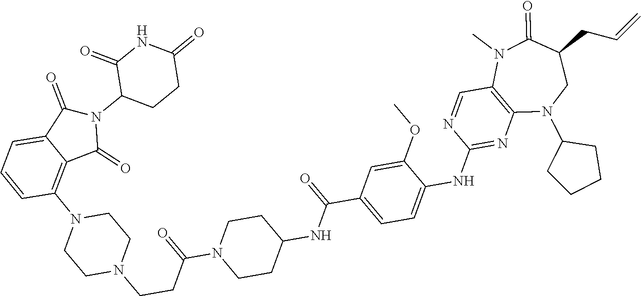 [SMILES: C=CC[C@H]1CN(C2CCCC2)c2nc(Nc3ccc(C(=O)NC4CCN(C(=O)CCN5CCN(c6cccc7c6C(=O)N(C6CCC(=O)NC6=O)C7=O)CC5)CC4)cc3OC)ncc2N(C)C1=O] |
| 71 | 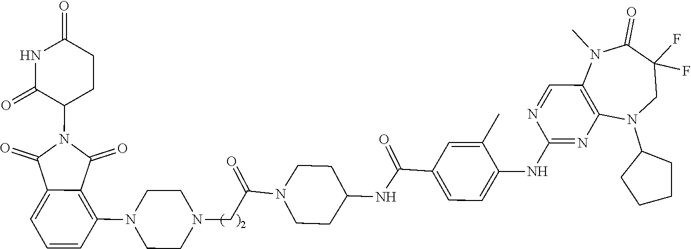 |
| 72 | 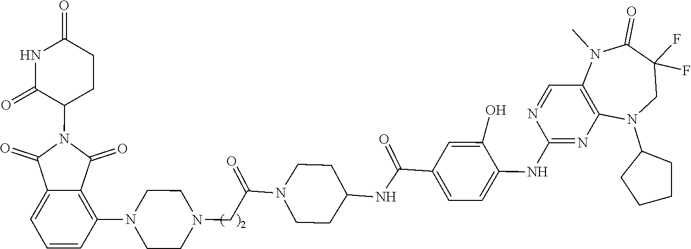 |
| 73 | 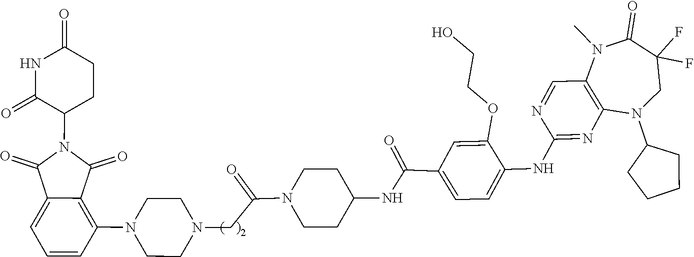 |
| 74 | 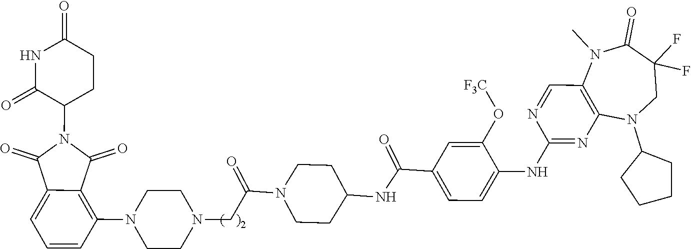 |
| 75 | 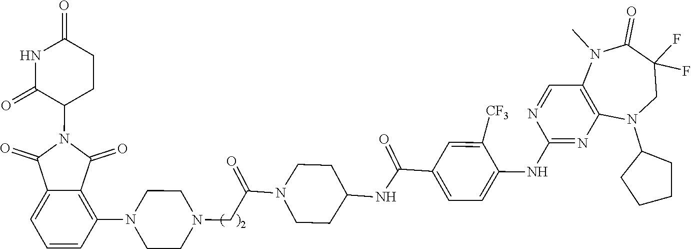 |
| 76 | 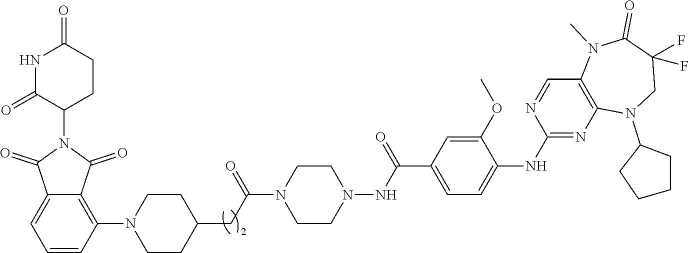 |
| 77 | 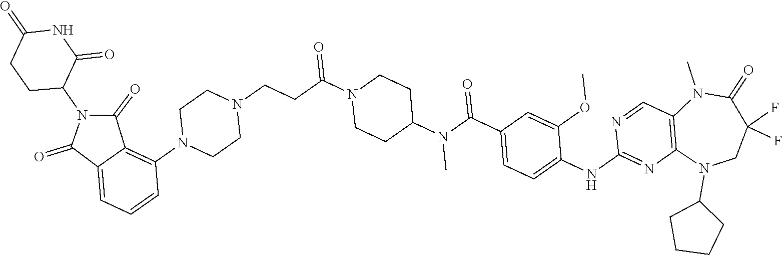 [SMILES: COc1cc(C(=O)N(C)C2CCN(C(=O)CCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 78 | 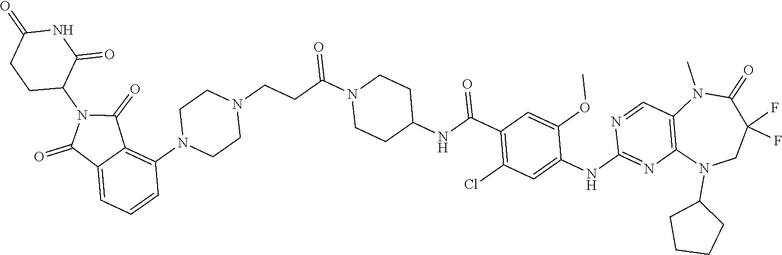 [SMILES: COc1cc(C(=O)NC2CCN(C(=O)CCN3CCN(c4cccc5c4C(=O)N(C4CCC(=O)NC4=O)C5=O)CC3)CC2)c(Cl)cc1Nc1ncc2c(n1)N(C1CCCC1)CC(F)(F)C(=O)N2C] |
| 79 | 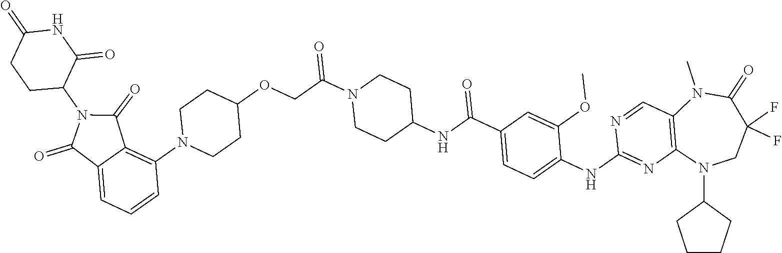 [SMILES: COc1cc(C(=O)NC2CCN(C(=O)COC3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 80 | 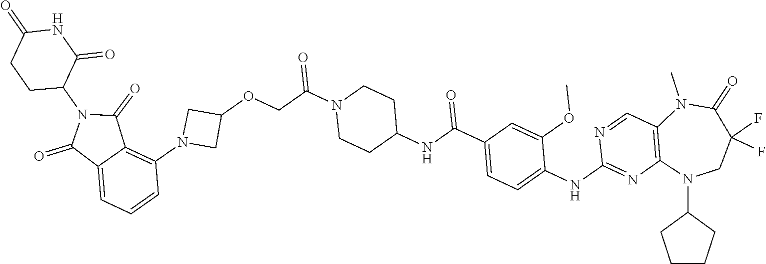 [SMILES: COc1cc(C(=O)NC2CCN(C(=O)COC3CN(c4cccc5c4C(=O)N(C4CCC(=O)NC4=O)C5=O)C3)CC2)ccc1Nc1ncc2c(n1)N(C1CCCC1)CC(F)(F)C(=O)N2C] |
| 81 | 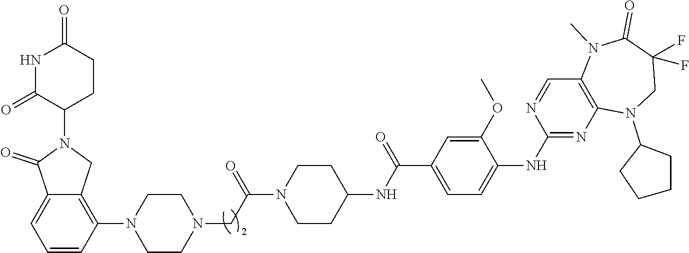 |
| 82 | 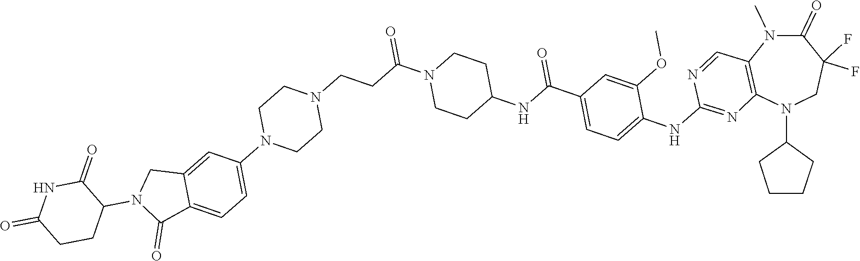 [SMILES: COc1cc(C(=O)NC2CCN(C(=O)CCN3CCN(c4ccc5c(c4)CN(C4CCC(=O)NC4=O)C5=O)CC3)CC2)ccc1Nc1ncc2c(n1)N(C1CCCC1)CC(F)(F)C(=O)N2C] |
| 83 | 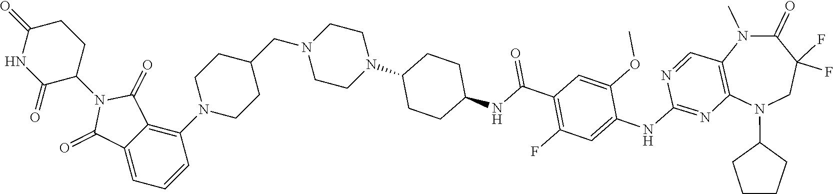 [SMILES: COc1cc(C(=O)N[C@H]2CC[C@H](N3CCN(CC4CCN(c5cccc6c5C(=O)N(C5CCC(=O)NC5=O)C6=O)CC4)CC3)CC2)c(F)cc1Nc1ncc2c(n1)N(C1CCCC1)CC(F)(F)C(=O)N2C] |
| 84 | 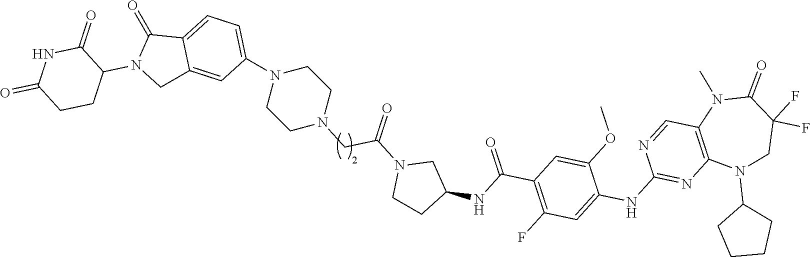 |
| 85 | 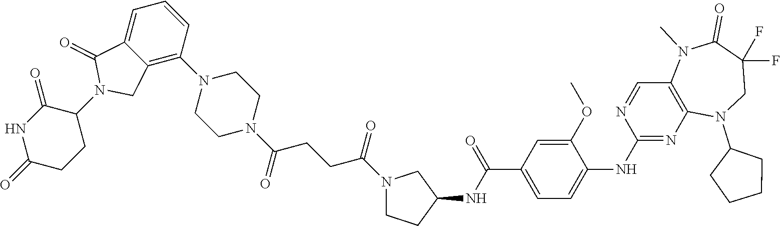 [SMILES: COc1cc(C(=O)N[C@H]2CCN(C(=O)CCC(=O)N3CCN(c4cccc5c4CN(C4CCC(=O)NC4=O)C5=O)CC3)C2)ccc1Nc1ncc2c(n1)N(C1CCCC1)CC(F)(F)C(=O)N2C] |
| 86 | 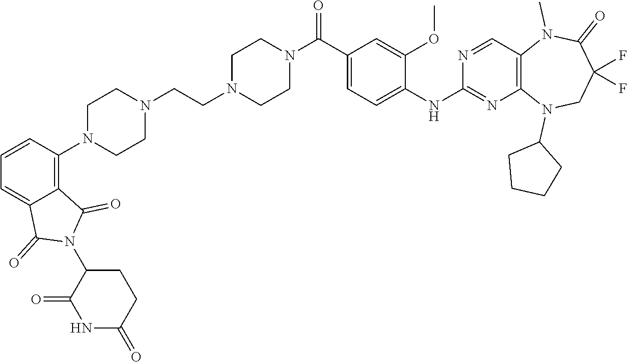 [SMILES: COc1cc(C(=O)N2CCN(CCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 87 | 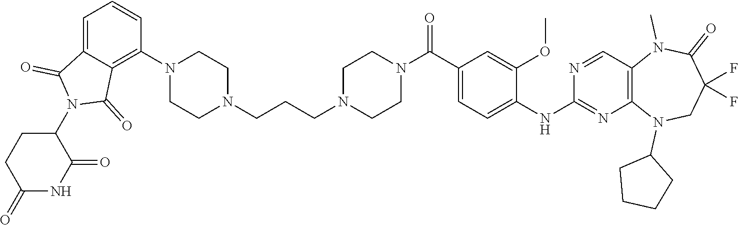 [SMILES: COc1cc(C(=O)N2CCN(CCCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 88 | 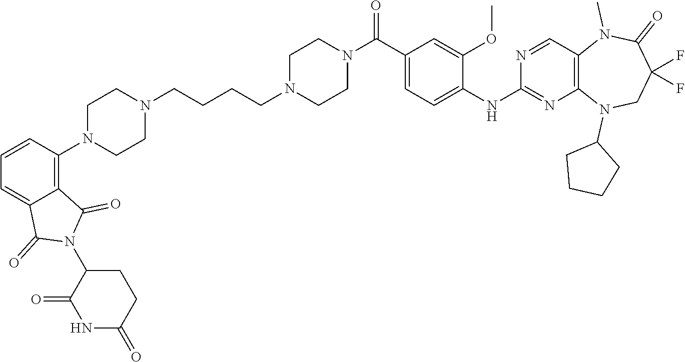 [SMILES: COc1cc(C(=O)N2CCN(CCCCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 89 | 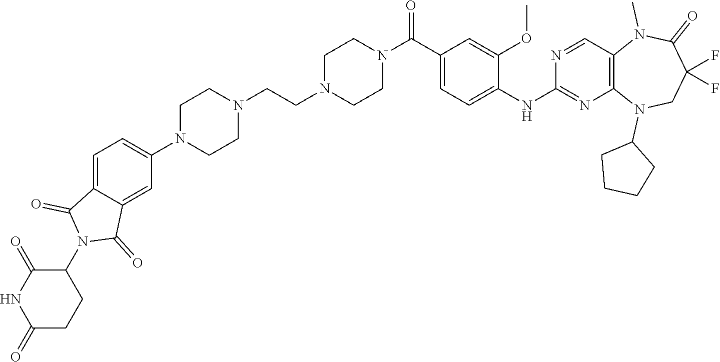 [SMILES: COc1cc(C(=O)N2CCN(CCN3CCN(c4ccc5c(c4)C(=O)N(C4CCC(=O)NC4=O)C5=O)CC3)CC2)ccc1Nc1ncc2c(n1)N(C1CCCC1)CC(F)(F)C(=O)N2C] |
| 90 | 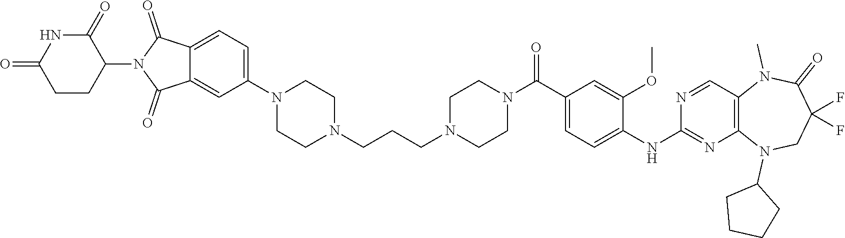 [SMILES: COc1cc(C(=O)N2CCN(CCCN3CCN(c4ccc5c(c4)C(=O)N(C4CCC(=O)NC4=O)C5=O)CC3)CC2)ccc1Nc1ncc2c(n1)N(C1CCCC1)CC(F)(F)C(=O)N2C] |
| 91 | 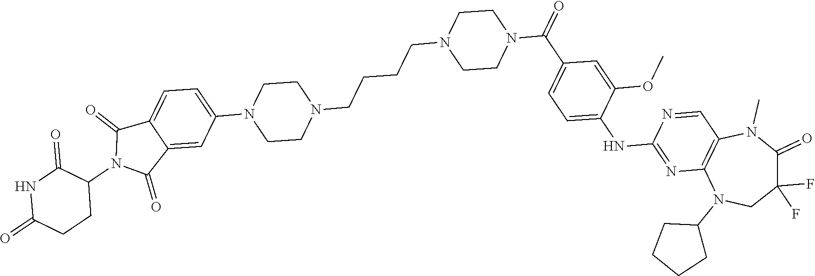 [SMILES: COc1cc(C(=O)N2CCN(CCCCN3CCN(c4ccc5c(c4)C(=O)N(C4CCC(=O)NC4=O)C5=O)CC3)CC2)ccc1Nc1ncc2c(n1)N(C1CCCC1)CC(F)(F)C(=O)N2C] |
| 92 | 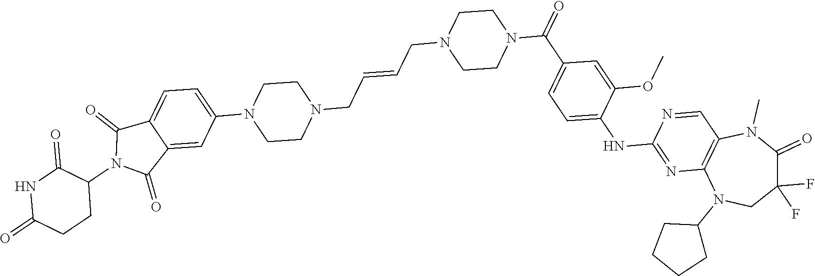 [SMILES: COc1cc(C(=O)N2CCN(C/C=C/CN3CCN(c4ccc5c(c4)C(=O)N(C4CCC(=O)NC4=O)C5=O)CC3)CC2)ccc1Nc1ncc2c(n1)N(C1CCCC1)CC(F)(F)C(=O)N2C] |
| 93 | 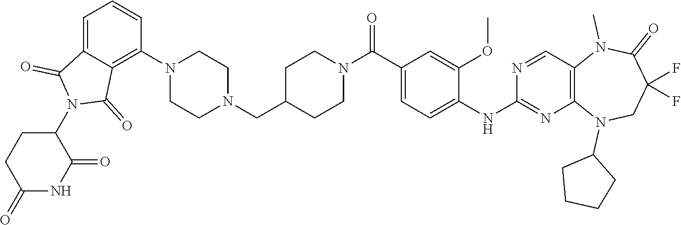 [SMILES: COc1cc(C(=O)N2CCC(CN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 94 | 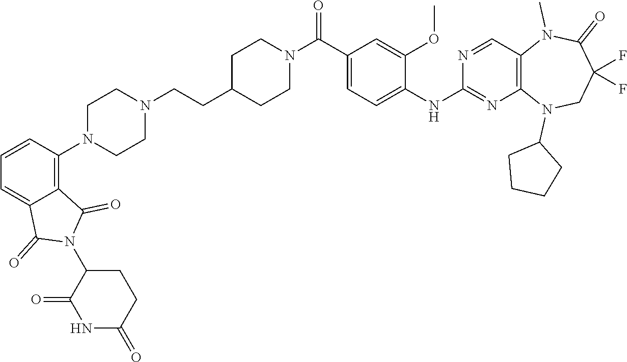 [SMILES: COc1cc(C(=O)N2CCC(CCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 95 | 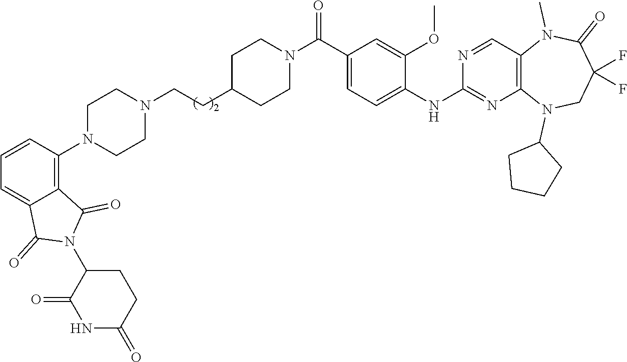 |
| 96 | 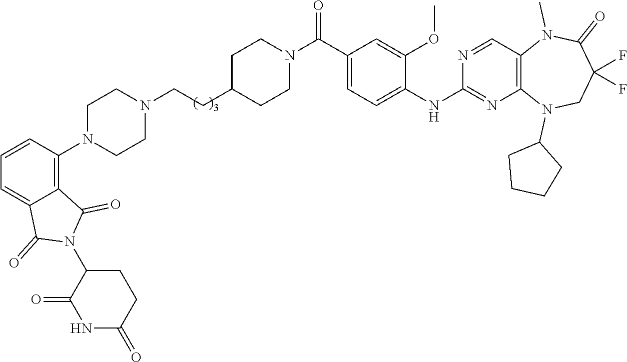 |
| 97 | 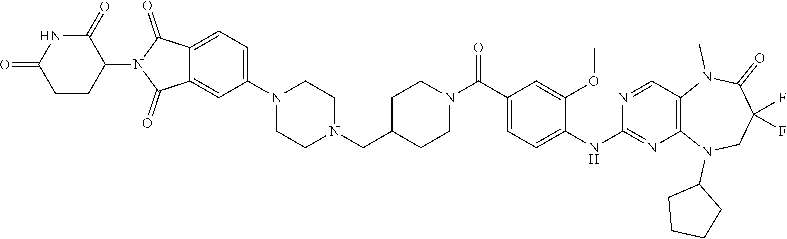 [SMILES: COc1cc(C(=O)N2CCC(CN3CCN(c4ccc5c(c4)C(=O)N(C4CCC(=O)NC4=O)C5=O)CC3)CC2)ccc1Nc1ncc2c(n1)N(C1CCCC1)CC(F)(F)C(=O)N2C] |
| 98 | 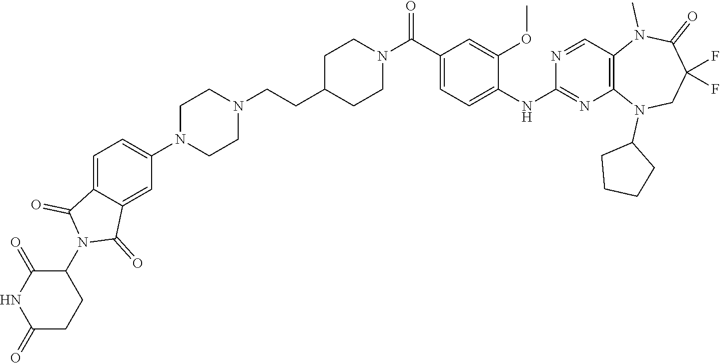 [SMILES: COc1cc(C(=O)N2CCC(CCN3CCN(c4ccc5c(c4)C(=O)N(C4CCC(=O)NC4=O)C5=O)CC3)CC2)ccc1Nc1ncc2c(n1)N(C1CCCC1)CC(F)(F)C(=O)N2C] |
| 99 | 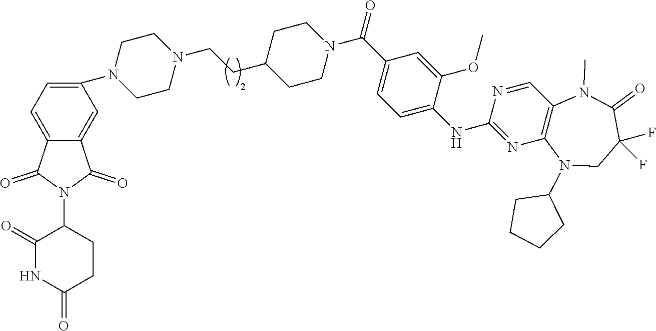 |
| 100 | 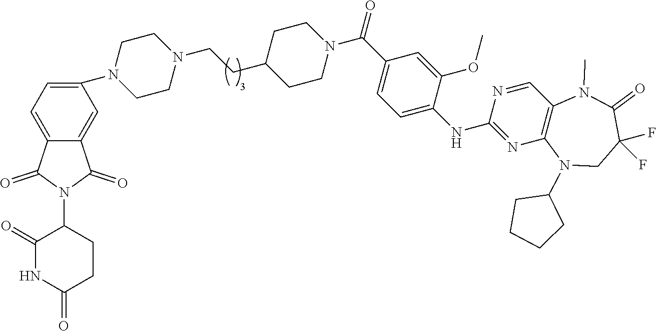 |
| 101 | 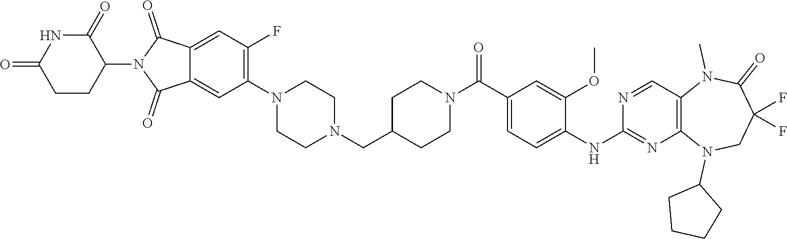 [SMILES: COc1cc(C(=O)N2CCC(CN3CCN(c4cc5c(cc4F)C(=O)N(C4CCC(=O)NC4=O)C5=O)CC3)CC2)ccc1Nc1ncc2c(n1)N(C1CCCC1)CC(F)(F)C(=O)N2C] |
| 102 | 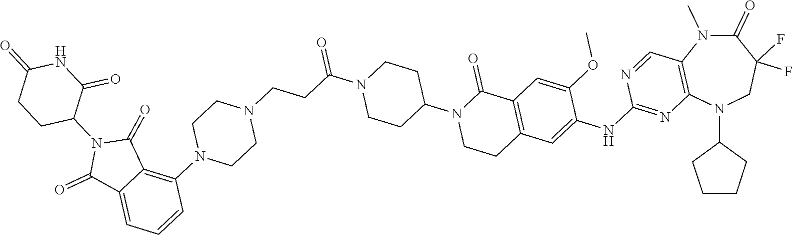 [SMILES: COc1cc2c(cc1Nc1ncc3c(n1)N(C1CCCC1)CC(F)(F)C(=O)N3C)CCN(C1CCN(C(=O)CCN3CCN(c4cccc5c4C(=O)N(C4CCC(=O)NC4=O)C5=O)CC3)CC1)C2=O] |
| 103 | 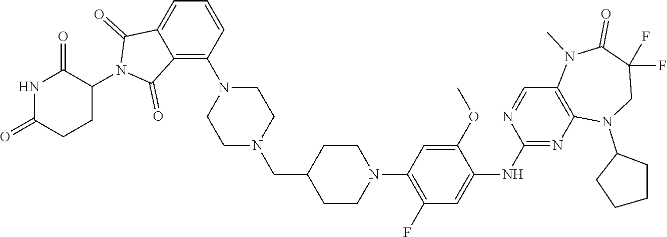 [SMILES: COc1cc(N2CCC(CN3CCN(c4cccc5c4C(=O)N(C4CCC(=O)NC4=O)C5=O)CC3)CC2)c(F)cc1Nc1ncc2c(n1)N(C1CCCC1)CC(F)(F)C(=O)N2C] |
| 104 | 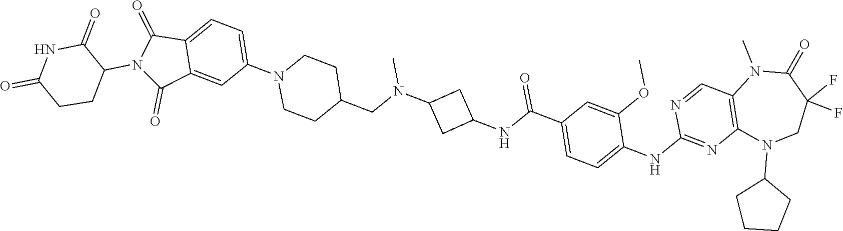 [SMILES: COc1cc(C(=O)NC2CC(N(C)CC3CCN(c4ccc5c(c4)C(=O)N(C4CCC(=O)NC4=O)C5=O)CC3)C2)ccc1Nc1ncc2c(n1)N(C1CCCC1)CC(F)(F)C(=O)N2C] |
| 105 | 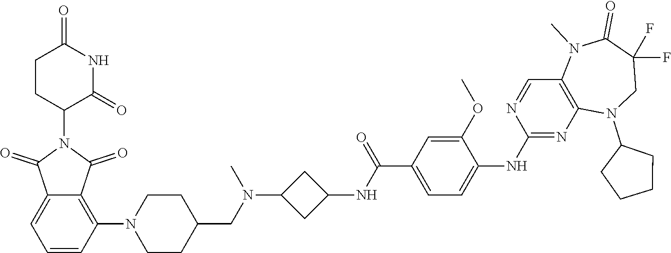 [SMILES: COc1cc(C(=O)NC2CC(N(C)CC3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 106 | 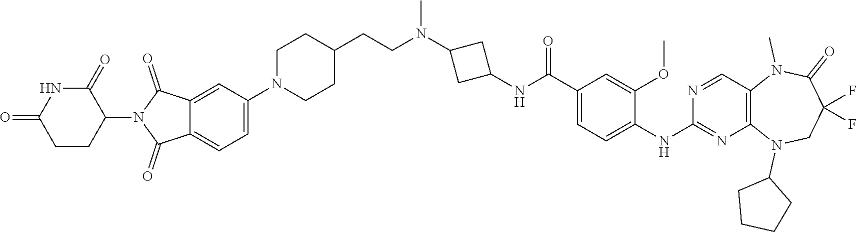 [SMILES: COc1cc(C(=O)NC2CC(N(C)CCC3CCN(c4ccc5c(c4)C(=O)N(C4CCC(=O)NC4=O)C5=O)CC3)C2)ccc1Nc1ncc2c(n1)N(C1CCCC1)CC(F)(F)C(=O)N2C] |
| 107 | 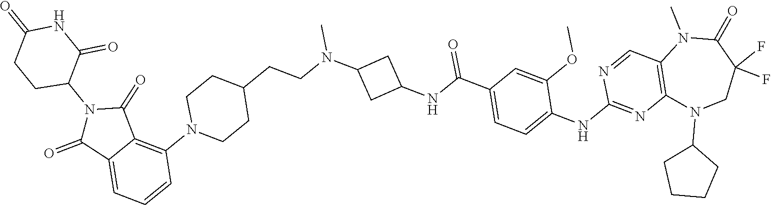 [SMILES: COc1cc(C(=O)NC2CC(N(C)CCC3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 108 | 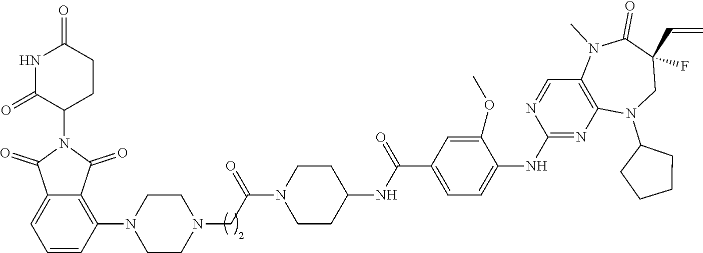 |
| 109 | 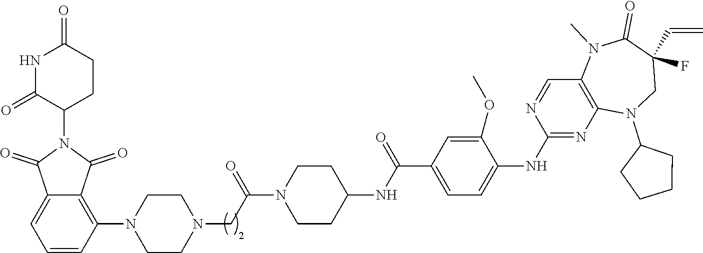 |
| 110 | 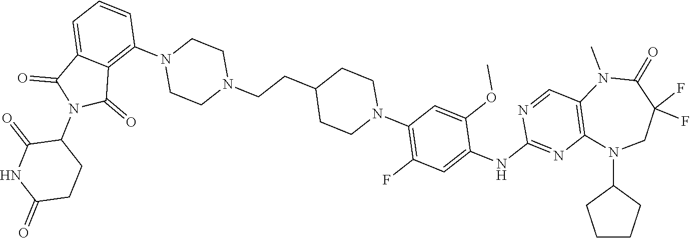 [SMILES: COc1cc(N2CCC(CCN3CCN(c4cccc5c4C(=O)N(C4CCC(=O)NC4=O)C5=O)CC3)CC2)c(F)cc1Nc1ncc2c(n1)N(C1CCCC1)CC(F)(F)C(=O)N2C] |
| 111 | 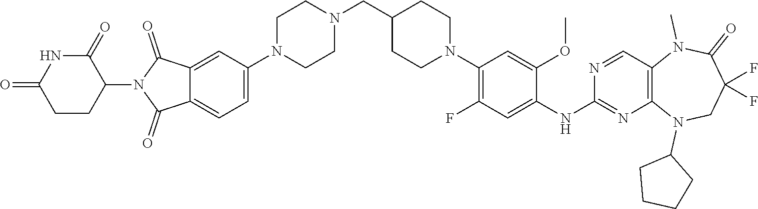 [SMILES: COc1cc(N2CCC(CN3CCN(c4ccc5c(c4)C(=O)N(C4CCC(=O)NC4=O)C5=O)CC3)CC2)c(F)cc1Nc1ncc2c(n1)N(C1CCCC1)CC(F)(F)C(=O)N2C] |
| 112 | 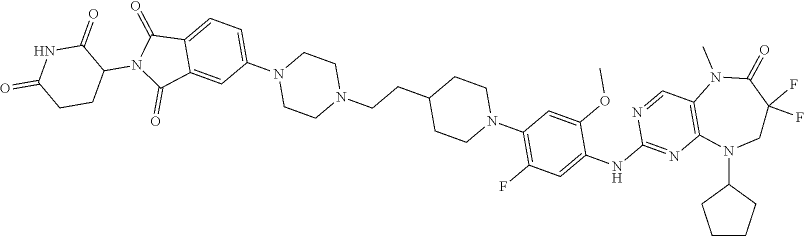 [SMILES: COc1cc(N2CCC(CCN3CCN(c4ccc5c(c4)C(=O)N(C4CCC(=O)NC4=O)C5=O)CC3)CC2)c(F)cc1Nc1ncc2c(n1)N(C1CCCC1)CC(F)(F)C(=O)N2C] |
| 113 | 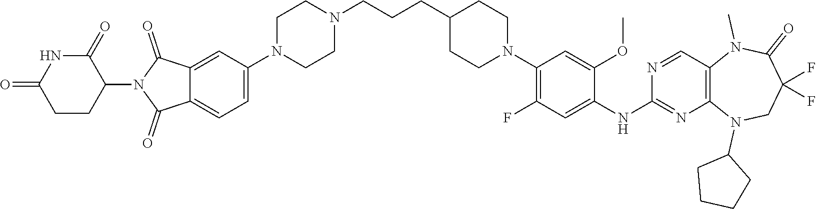 [SMILES: COc1cc(N2CCC(CCCN3CCN(c4ccc5c(c4)C(=O)N(C4CCC(=O)NC4=O)C5=O)CC3)CC2)c(F)cc1Nc1ncc2c(n1)N(C1CCCC1)CC(F)(F)C(=O)N2C] |
| 114 | 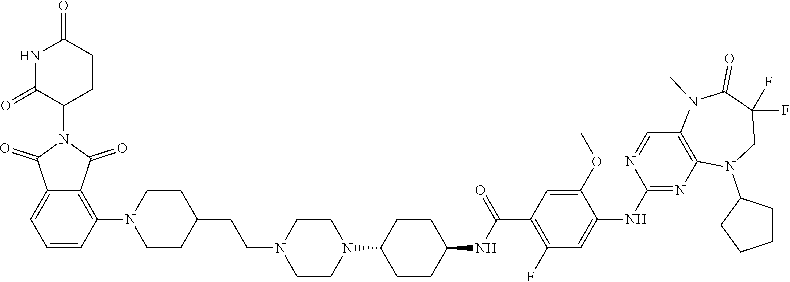 [SMILES: COc1cc(C(=O)N[C@H]2CC[C@H](N3CCN(CCC4CCN(c5cccc6c5C(=O)N(C5CCC(=O)NC5=O)C6=O)CC4)CC3)CC2)c(F)cc1Nc1ncc2c(n1)N(C1CCCC1)CC(F)(F)C(=O)N2C] |
| 115 | 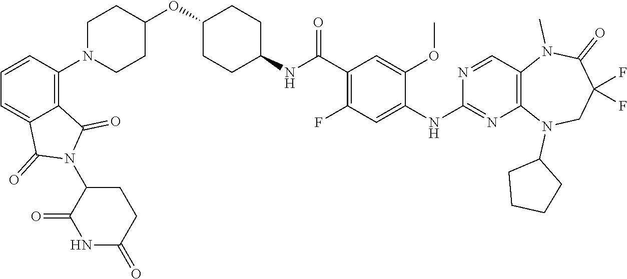 [SMILES: COc1cc(C(=O)N[C@H]2CC[C@H](OC3CCN(c4cccc5c4C(=O)N(C4CCC(=O)NC4=O)C5=O)CC3)CC2)c(F)cc1Nc1ncc2c(n1)N(C1CCCC1)CC(F)(F)C(=O)N2C] |
| 116 | 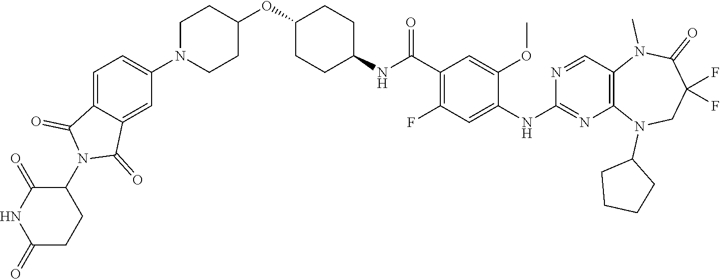 [SMILES: COc1cc(C(=O)N[C@H]2CC[C@H](OC3CCN(c4ccc5c(c4)C(=O)N(C4CCC(=O)NC4=O)C5=O)CC3)CC2)c(F)cc1Nc1ncc2c(n1)N(C1CCCC1)CC(F)(F)C(=O)N2C] |
| 117 | 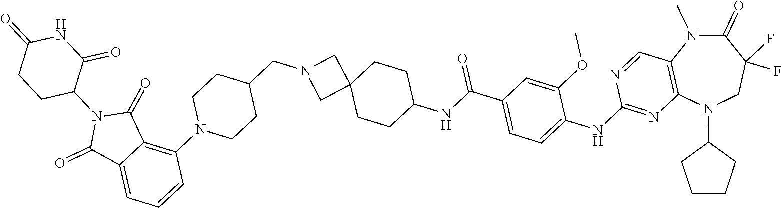 [SMILES: COc1cc(C(=O)NC2CCC3(CC2)CN(CC2CCN(c4cccc5c4C(=O)N(C4CCC(=O)NC4=O)C5=O)CC2)C3)ccc1Nc1ncc2c(n1)N(C1CCCC1)CC(F)(F)C(=O)N2C] |
| 118 | 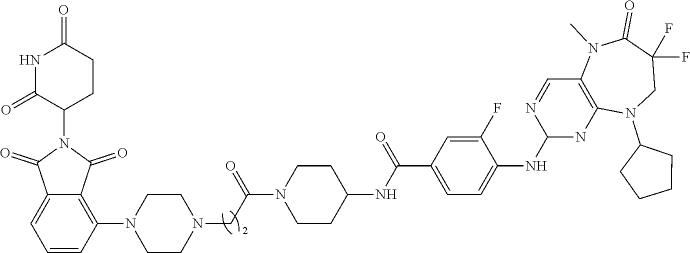 |
| 119 | 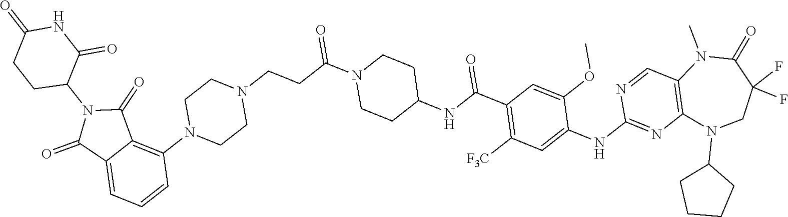 [SMILES: COc1cc(C(=O)NC2CCN(C(=O)CCN3CCN(c4cccc5c4C(=O)N(C4CCC(=O)NC4=O)C5=O)CC3)CC2)c(C(F)(F)F)cc1Nc1ncc2c(n1)N(C1CCCC1)CC(F)(F)C(=O)N2C] |
| 120 | 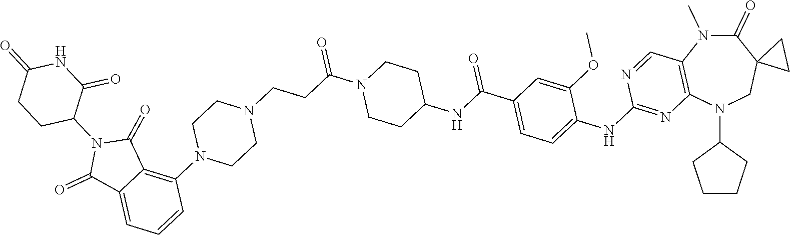 [SMILES: COc1cc(C(=O)NC2CCN(C(=O)CCN3CCN(c4cccc5c4C(=O)N(C4CCC(=O)NC4=O)C5=O)CC3)CC2)ccc1Nc1ncc2c(n1)N(C1CCCC1)CC1(CC1)C(=O)N2C] |
| 121 | 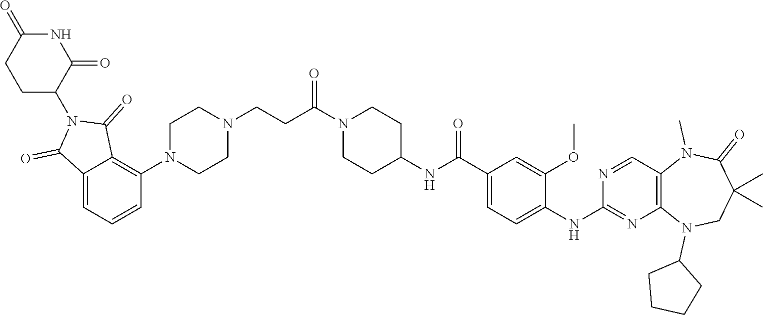 [SMILES: COc1cc(C(=O)NC2CCN(C(=O)CCN3CCN(c4cccc5c4C(=O)N(C4CCC(=O)NC4=O)C5=O)CC3)CC2)ccc1Nc1ncc2c(n1)N(C1CCCC1)CC(C)(C)C(=O)N2C] |
| 122 | 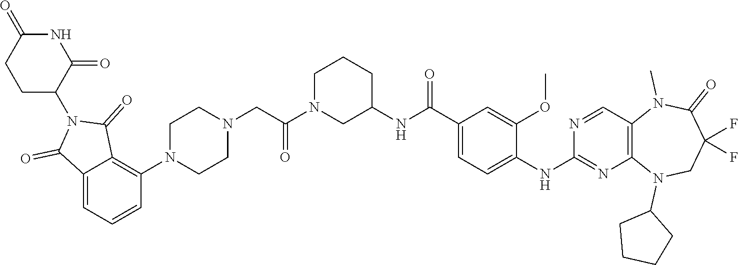 [SMILES: COc1cc(C(=O)NC2CCCN(C(=O)CN3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 123 | 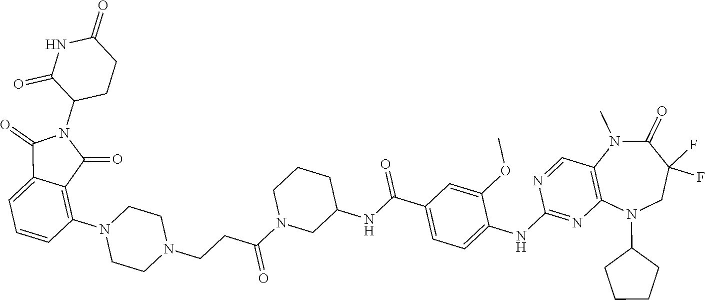 [SMILES: COc1cc(C(=O)NC2CCCN(C(=O)CCN3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 124 | 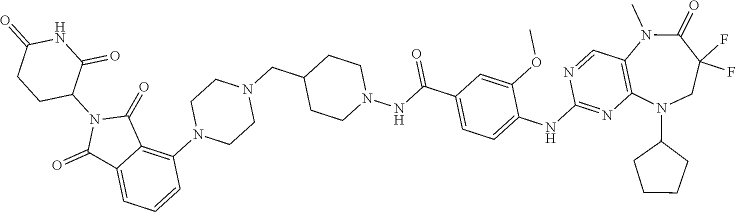 [SMILES: COc1cc(C(=O)NN2CCC(CN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 125 | 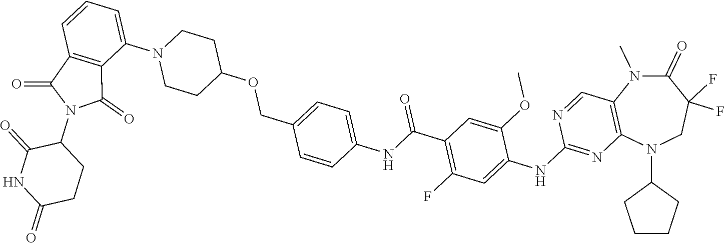 [SMILES: COc1cc(C(=O)Nc2ccc(COC3CCN(c4cccc5c4C(=O)N(C4CCC(=O)NC4=O)C5=O)CC3)cc2)c(F)cc1Nc1ncc2c(n1)N(C1CCCC1)CC(F)(F)C(=O)N2C] |
| 126 | 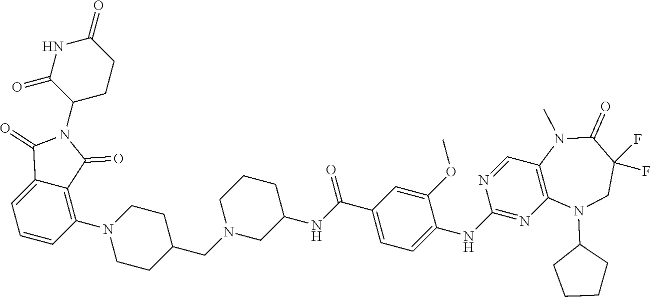 [SMILES: COc1cc(C(=O)NC2CCCN(CC3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 127 | 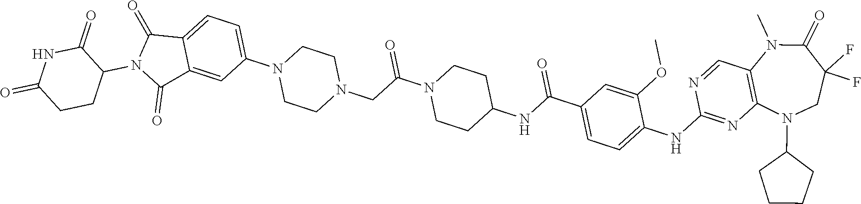 [SMILES: COc1cc(C(=O)NC2CCN(C(=O)CN3CCN(c4ccc5c(c4)C(=O)N(C4CCC(=O)NC4=O)C5=O)CC3)CC2)ccc1Nc1ncc2c(n1)N(C1CCCC1)CC(F)(F)C(=O)N2C] |
| 128 | 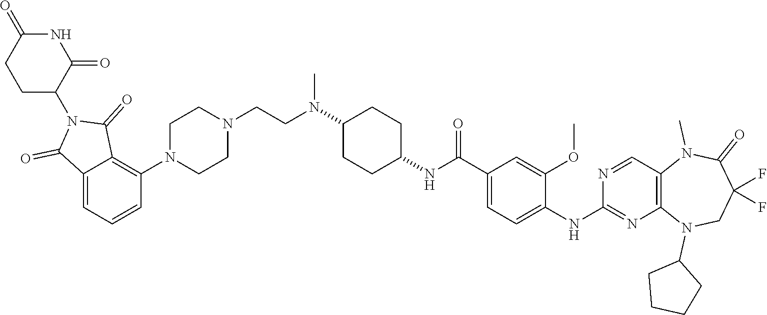 [SMILES: COc1cc(C(=O)N[C@H]2CC[C@@H](N(C)CCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 129 | 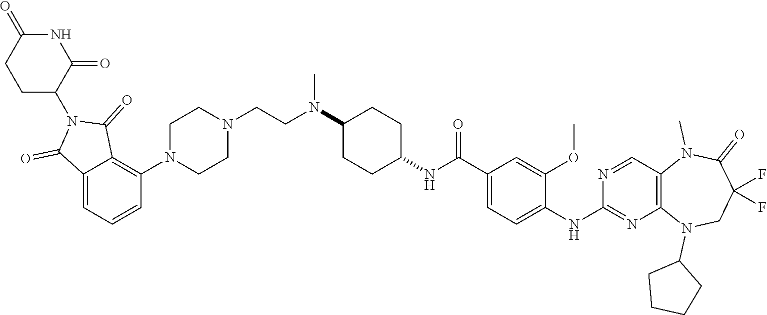 [SMILES: COc1cc(C(=O)N[C@H]2CC[C@H](N(C)CCN3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 130 | 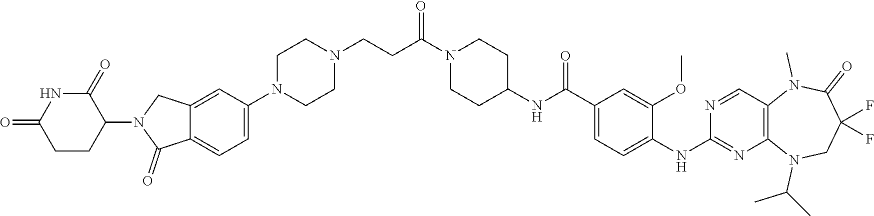 [SMILES: COc1cc(C(=O)NC2CCN(C(=O)CCN3CCN(c4ccc5c(c4)CN(C4CCC(=O)NC4=O)C5=O)CC3)CC2)ccc1Nc1ncc2c(n1)N(C(C)C)CC(F)(F)C(=O)N2C] |
| 131 | 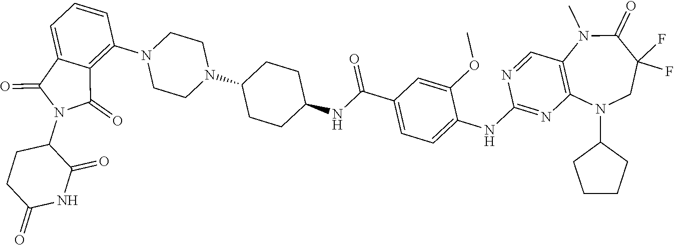 [SMILES: COc1cc(C(=O)N[C@H]2CC[C@H](N3CCN(c4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 132 | 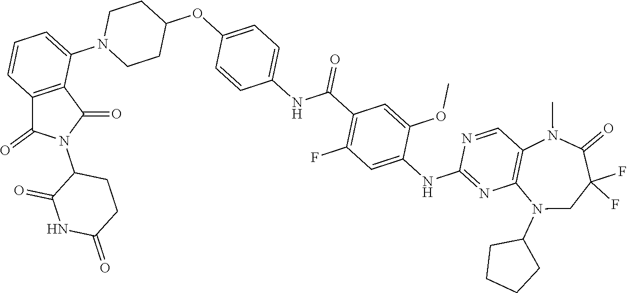 [SMILES: COc1cc(C(=O)Nc2ccc(OC3CCN(c4cccc5c4C(=O)N(C4CCC(=O)NC4=O)C5=O)CC3)cc2)c(F)cc1Nc1ncc2c(n1)N(C1CCCC1)CC(F)(F)C(=O)N2C] |
| 133 | 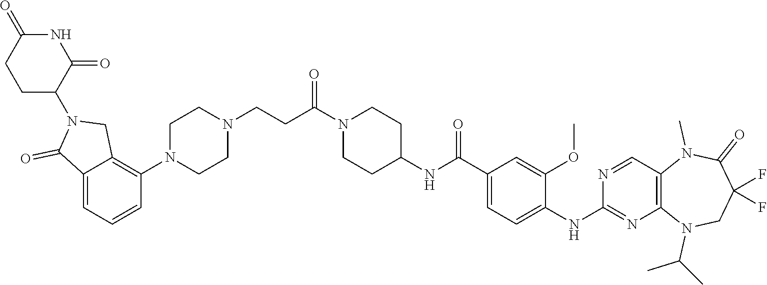 [SMILES: COc1cc(C(=O)NC2CCN(C(=O)CCN3CCN(c4cccc5c4CN(C4CCC(=O)NC4=O)C5=O)CC3)CC2)ccc1Nc1ncc2c(n1)N(C(C)C)CC(F)(F)C(=O)N2C] |
| 134 | 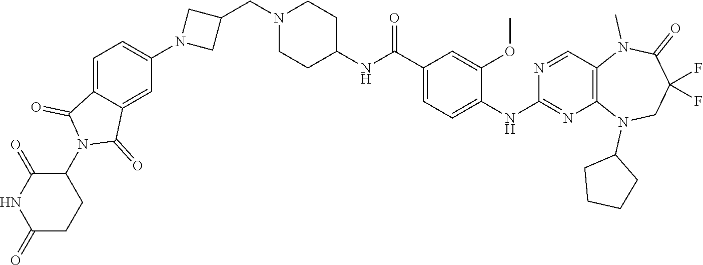 [SMILES: COc1cc(C(=O)NC2CCN(CC3CN(c4ccc5c(c4)C(=O)N(C4CCC(=O)NC4=O)C5=O)C3)CC2)ccc1Nc1ncc2c(n1)N(C1CCCC1)CC(F)(F)C(=O)N2C] |
| 135 | 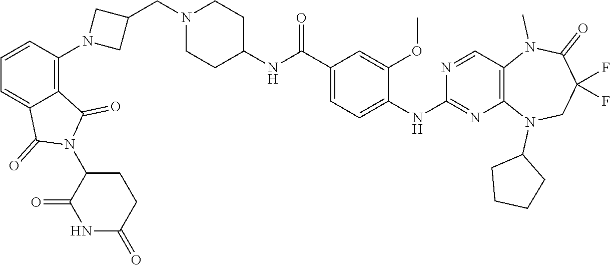 [SMILES: COc1cc(C(=O)NC2CCN(CC3CN(c4cccc5c4C(=O)N(C4CCC(=O)NC4=O)C5=O)C3)CC2)ccc1Nc1ncc2c(n1)N(C1CCCC1)CC(F)(F)C(=O)N2C] |
| 136 | 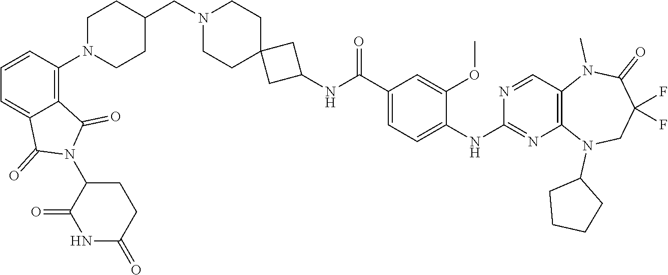 [SMILES: COc1cc(C(=O)NC2CC3(CCN(CC4CCN(c5cccc6c5C(=O)N(C5CCC(=O)NC5=O)C6=O)CC4)CC3)C2)ccc1Nc1ncc2c(n1)N(C1CCCC1)CC(F)(F)C(=O)N2C] |
| 137 | 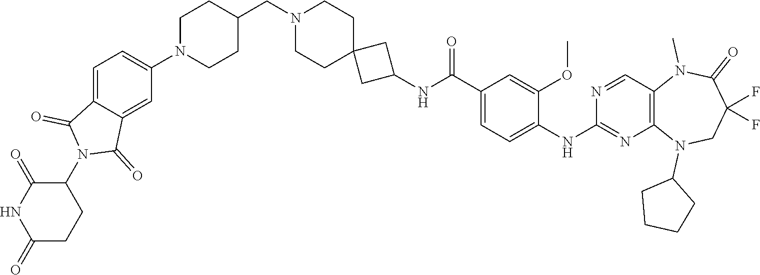 [SMILES: COc1cc(C(=O)NC2CC3(CCN(CC4CCN(c5ccc6c(c5)C(=O)N(C5CCC(=O)NC5=O)C6=O)CC4)CC3)C2)ccc1Nc1ncc2c(n1)N(C1CCCC1)CC(F)(F)C(=O)N2C] |
| 138 | 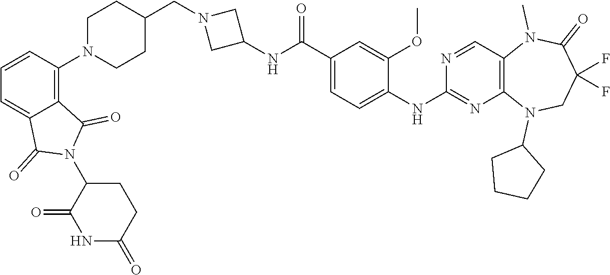 [SMILES: COc1cc(C(=O)NC2CN(CC3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 139 | 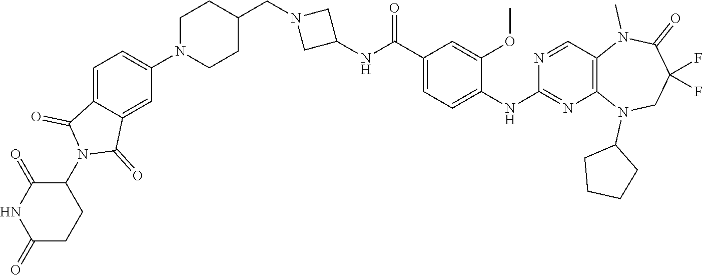 [SMILES: COc1cc(C(=O)NC2CN(CC3CCN(c4ccc5c(c4)C(=O)N(C4CCC(=O)NC4=O)C5=O)CC3)C2)ccc1Nc1ncc2c(n1)N(C1CCCC1)CC(F)(F)C(=O)N2C] |
| 140 | 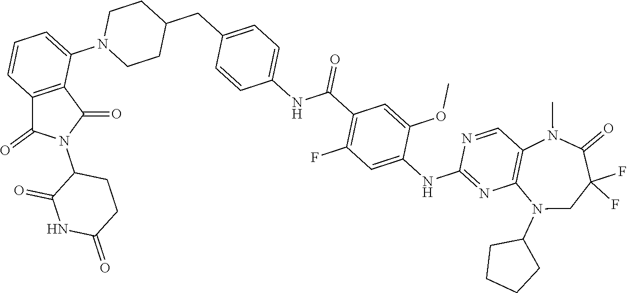 [SMILES: COc1cc(C(=O)Nc2ccc(CC3CCN(c4cccc5c4C(=O)N(C4CCC(=O)NC4=O)C5=O)CC3)cc2)c(F)cc1Nc1ncc2c(n1)N(C1CCCC1)CC(F)(F)C(=O)N2C] |
| 141 | 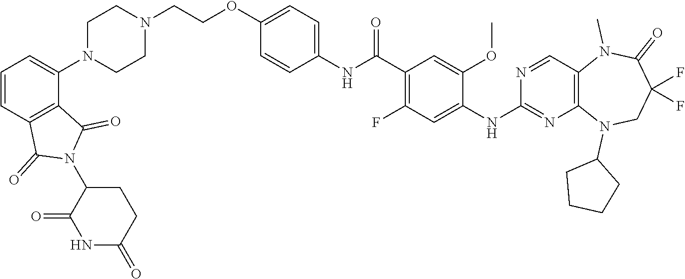 [SMILES: COc1cc(C(=O)Nc2ccc(OCCN3CCN(c4cccc5c4C(=O)N(C4CCC(=O)NC4=O)C5=O)CC3)cc2)c(F)cc1Nc1ncc2c(n1)N(C1CCCC1)CC(F)(F)C(=O)N2C] |
| 142 | 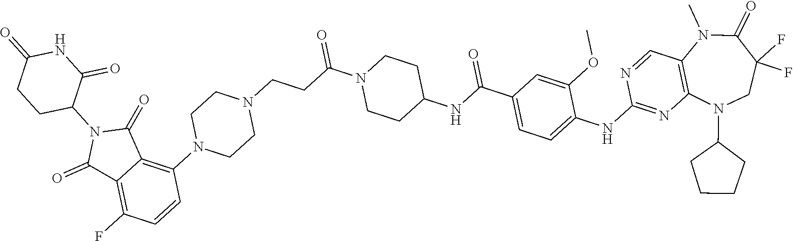 [SMILES: COc1cc(C(=O)NC2CCN(C(=O)CCN3CCN(c4ccc(F)c5c4C(=O)N(C4CCC(=O)NC4=O)C5=O)CC3)CC2)ccc1Nc1ncc2c(n1)N(C1CCCC1)CC(F)(F)C(=O)N2C] |
| 143 | 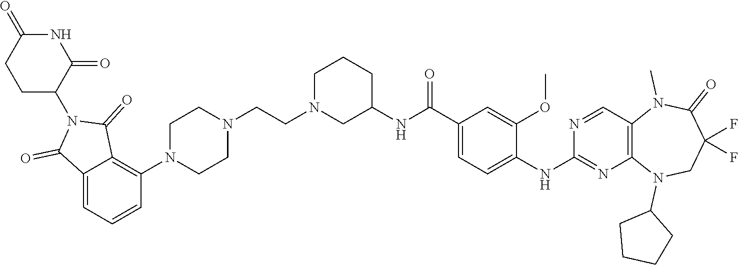 [SMILES: COc1cc(C(=O)NC2CCCN(CCN3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 144 | 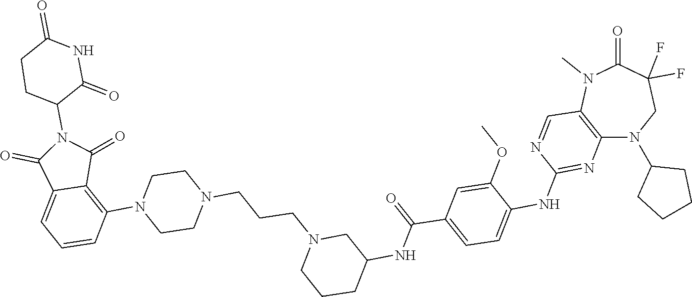 [SMILES: COc1cc(C(=O)NC2CCCN(CCCN3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 145 | 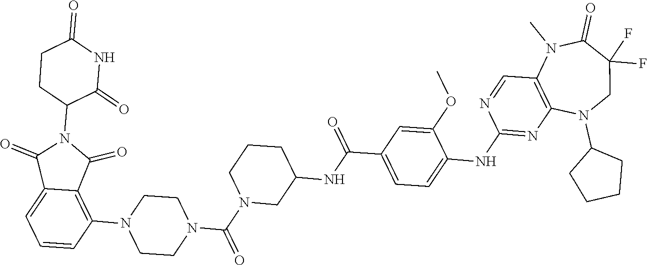 [SMILES: COc1cc(C(=O)NC2CCCN(C(=O)N3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 146 | 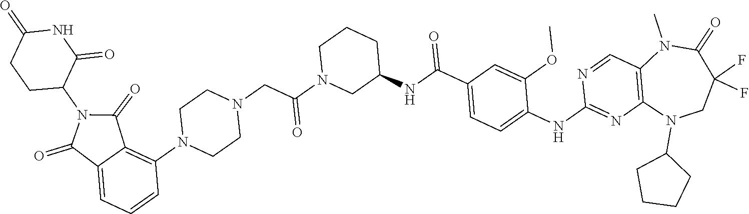 [SMILES: COc1cc(C(=O)N[C@@H]2CCCN(C(=O)CN3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 147 | 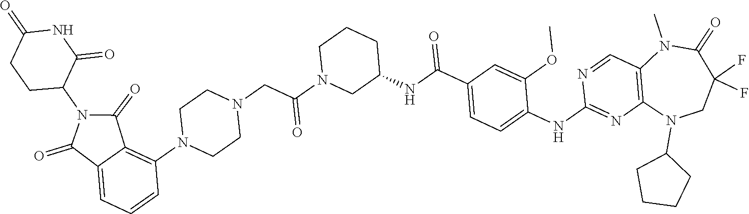 [SMILES: COc1cc(C(=O)N[C@H]2CCCN(C(=O)CN3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 148 | 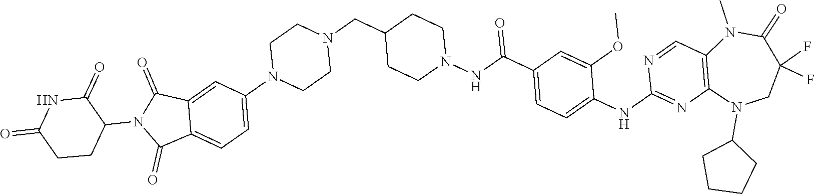 [SMILES: COc1cc(C(=O)NN2CCC(CN3CCN(c4ccc5c(c4)C(=O)N(C4CCC(=O)NC4=O)C5=O)CC3)CC2)ccc1Nc1ncc2c(n1)N(C1CCCC1)CC(F)(F)C(=O)N2C] |
| 149 | 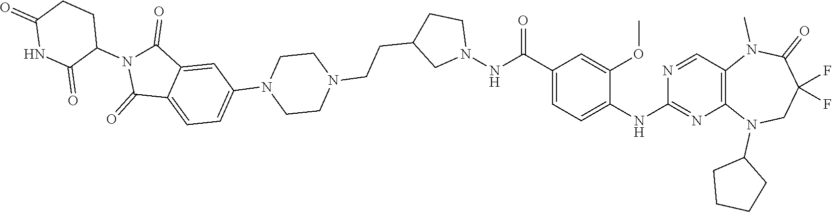 [SMILES: COc1cc(C(=O)NN2CCC(CCN3CCN(c4ccc5c(c4)C(=O)N(C4CCC(=O)NC4=O)C5=O)CC3)C2)ccc1Nc1ncc2c(n1)N(C1CCCC1)CC(F)(F)C(=O)N2C] |
| 150 | 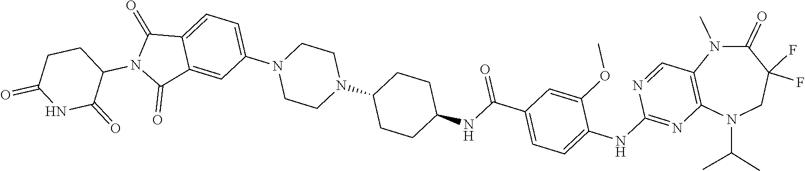 [SMILES: COc1cc(C(=O)N[C@H]2CC[C@H](N3CCN(c4ccc5c(c4)C(=O)N(C4CCC(=O)NC4=O)C5=O)CC3)CC2)ccc1Nc1ncc2c(n1)N(C(C)C)CC(F)(F)C(=O)N2C] |
| 151 | 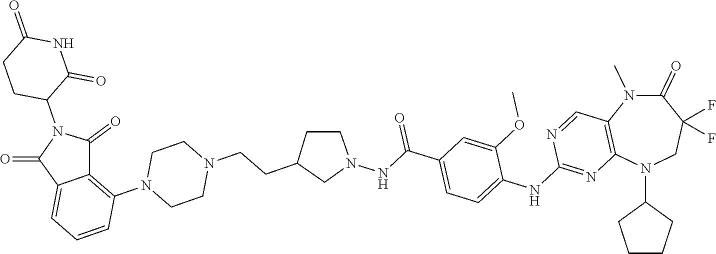 [SMILES: COc1cc(C(=O)NN2CCC(CCN3CCN(c4cccc5c4C(=O)N(C4CCC(=O)NC4=O)C5=O)CC3)C2)ccc1Nc1ncc2c(n1)N(C1CCCC1)CC(F)(F)C(=O)N2C] |
| 152 | 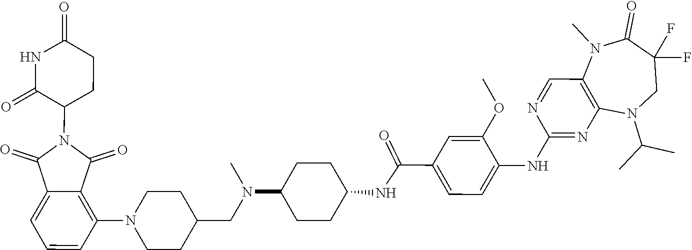 [SMILES: COc1cc(C(=O)N[C@H]2CC[C@H](N(C)CC3CCN(c4cccc5c4C(=O)N(C4CCC(=O)NC4=O)C5=O)CC3)CC2)ccc1Nc1ncc2c(n1)N(C(C)C)CC(F)(F)C(=O)N2C] |
| 153 | 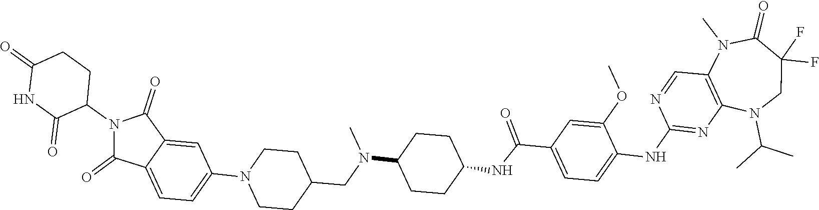 [SMILES: COc1cc(C(=O)N[C@H]2CC[C@H](N(C)CC3CCN(c4ccc5c(c4)C(=O)N(C4CCC(=O)NC4=O)C5=O)CC3)CC2)ccc1Nc1ncc2c(n1)N(C(C)C)CC(F)(F)C(=O)N2C] |
| 154 | 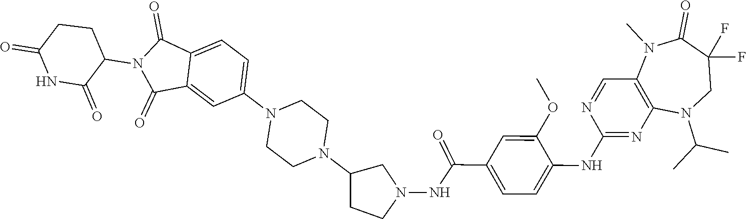 [SMILES: COc1cc(C(=O)NN2CCC(N3CCN(c4ccc5c(c4)C(=O)N(C4CCC(=O)NC4=O)C5=O)CC3)C2)ccc1Nc1ncc2c(n1)N(C(C)C)CC(F)(F)C(=O)N2C] |
| 155 | 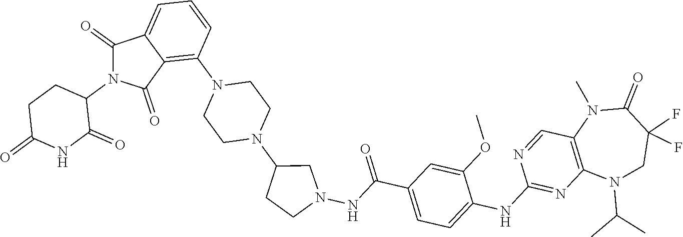 [SMILES: COc1cc(C(=O)NN2CCC(N3CCN(c4cccc5c4C(=O)N(C4CCC(=O)NC4=O)C5=O)CC3)C2)ccc1Nc1ncc2c(n1)N(C(C)C)CC(F)(F)C(=O)N2C] |
| 156 | 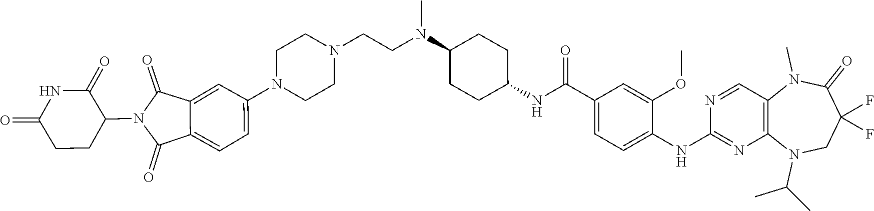 [SMILES: COc1cc(C(=O)N[C@H]2CC[C@H](N(C)CCN3CCN(c4ccc5c(c4)C(=O)N(C4CCC(=O)NC4=O)C5=O)CC3)CC2)ccc1Nc1ncc2c(n1)N(C(C)C)CC(F)(F)C(=O)N2C] |
| 157 | 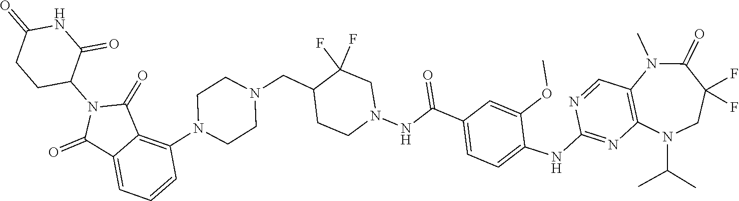 [SMILES: COc1cc(C(=O)NN2CCC(CN3CCN(c4cccc5c4C(=O)N(C4CCC(=O)NC4=O)C5=O)CC3)C(F)(F)C2)ccc1Nc1ncc2c(n1)N(C(C)C)CC(F)(F)C(=O)N2C] |
| 158 | 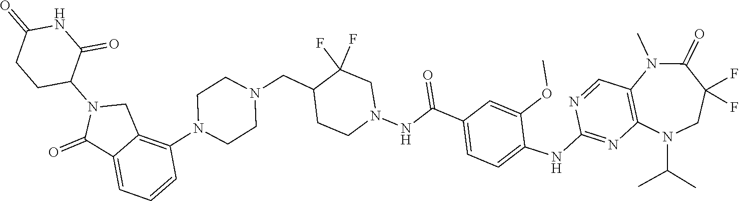 [SMILES: COc1cc(C(=O)NN2CCC(CN3CCN(c4cccc5c4CN(C4CCC(=O)NC4=O)C5=O)CC3)C(F)(F)C2)ccc1Nc1ncc2c(n1)N(C(C)C)CC(F)(F)C(=O)N2C] |
| 159 | 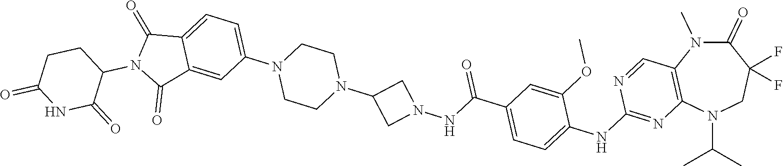 [SMILES: COc1cc(C(=O)NN2CC(N3CCN(c4ccc5c(c4)C(=O)N(C4CCC(=O)NC4=O)C5=O)CC3)C2)ccc1Nc1ncc2c(n1)N(C(C)C)CC(F)(F)C(=O)N2C] |
| 160 | 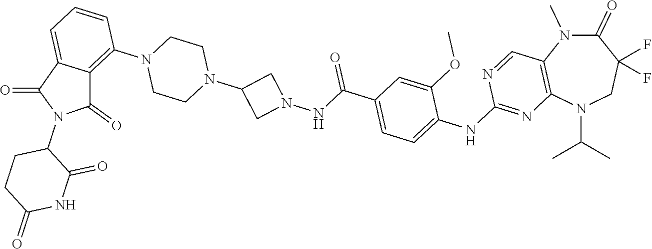 [SMILES: COc1cc(C(=O)NN2CC(N3CCN(c4cccc5c4C(=O)N(C4CCC(=O)NC4=O)C5=O)CC3)C2)ccc1Nc1ncc2c(n1)N(C(C)C)CC(F)(F)C(=O)N2C] |
| 161 | 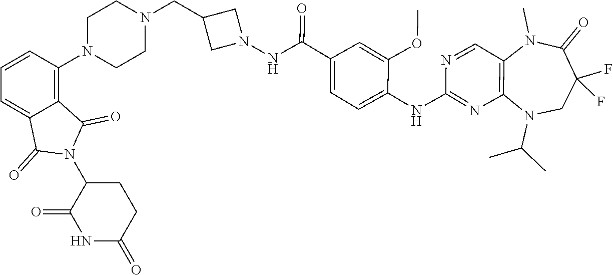 [SMILES: COc1cc(C(=O)NN2CC(CN3CCN(c4cccc5c4C(=O)N(C4CCC(=O)NC4=O)C5=O)CC3)C2)ccc1Nc1ncc2c(n1)N(C(C)C)CC(F)(F)C(=O)N2C] |
| 162 | 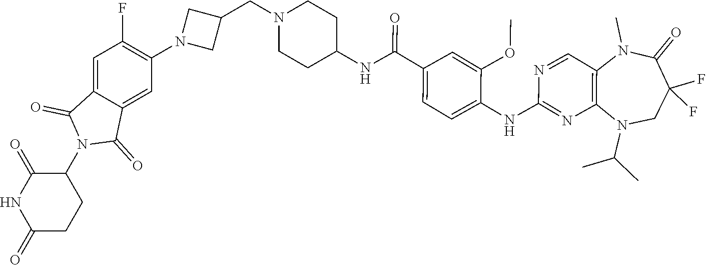 [SMILES: COc1cc(C(=O)NC2CCN(CC3CN(c4cc5c(cc4F)C(=O)N(C4CCC(=O)NC4=O)C5=O)C3)CC2)ccc1Nc1ncc2c(n1)N(C(C)C)CC(F)(F)C(=O)N2C] |
| 163 | 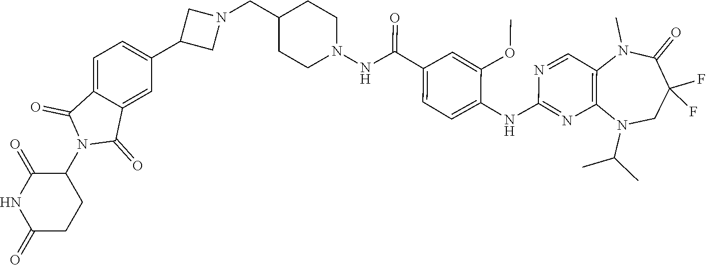 [SMILES: COc1cc(C(=O)NN2CCC(CN3CC(c4ccc5c(c4)C(=O)N(C4CCC(=O)NC4=O)C5=O)C3)CC2)ccc1Nc1ncc2c(n1)N(C(C)C)CC(F)(F)C(=O)N2C] |
| 164 | 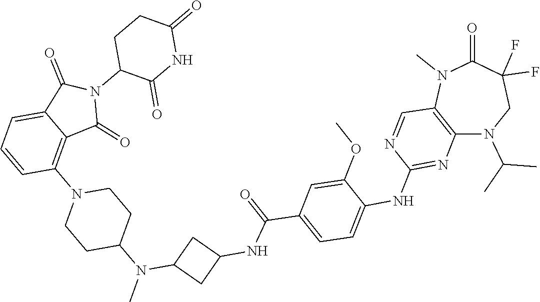 [SMILES: COc1cc(C(=O)NC2CC(N(C)C3CCN(c4cccc5c4C(=O)N(C4CCC(=O)NC4=O)C5=O)CC3)C2)ccc1Nc1ncc2c(n1)N(C(C)C)CC(F)(F)C(=O)N2C] |
| 165 | 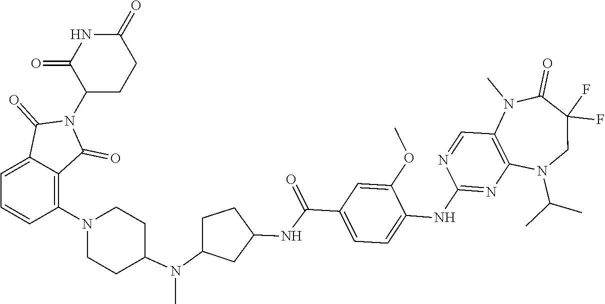 [SMILES: COc1cc(C(=O)NC2CCC(N(C)C3CCN(c4cccc5c4C(=O)N(C4CCC(=O)NC4=O)C5=O)CC3)C2)ccc1Nc1ncc2c(n1)N(C(C)C)CC(F)(F)C(=O)N2C] |
| 166 | 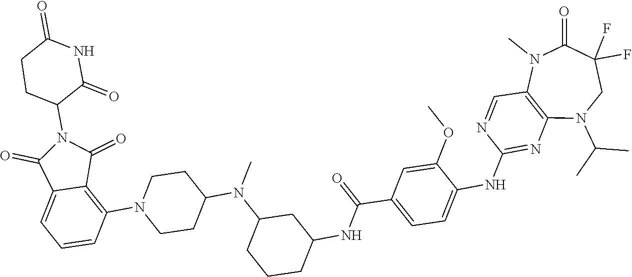 [SMILES: COc1cc(C(=O)NC2CCCC(N(C)C3CCN(c4cccc5c4C(=O)N(C4CCC(=O)NC4=O)C5=O)CC3)C2)ccc1Nc1ncc2c(n1)N(C(C)C)CC(F)(F)C(=O)N2C] |
| 167 | 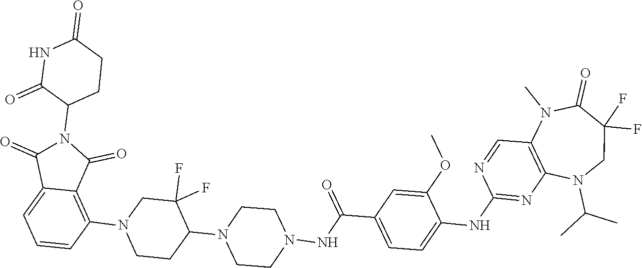 [SMILES: COc1cc(C(=O)NN2CCN(C3CCN(c4cccc5c4C(=O)N(C4CCC(=O)NC4=O)C5=O)CC3(F)F)CC2)ccc1Nc1ncc2c(n1)N(C(C)C)CC(F)(F)C(=O)N2C] |
| 168 | 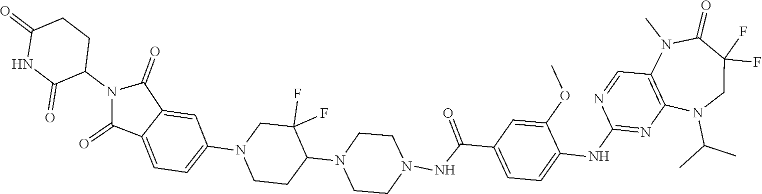 [SMILES: COc1cc(C(=O)NN2CCN(C3CCN(c4ccc5c(c4)C(=O)N(C4CCC(=O)NC4=O)C5=O)CC3(F)F)CC2)ccc1Nc1ncc2c(n1)N(C(C)C)CC(F)(F)C(=O)N2C] |
| 169 | 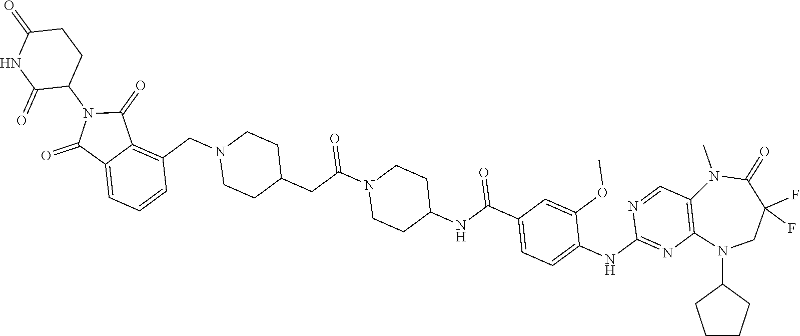 [SMILES: COc1cc(C(=O)NC2CCN(C(=O)CC3CCN(Cc4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 170 | 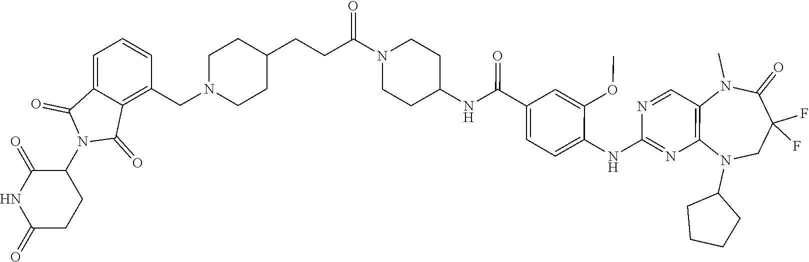 [SMILES: COc1cc(C(=O)NC2CCN(C(=O)CCC3CCN(Cc4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 171 | 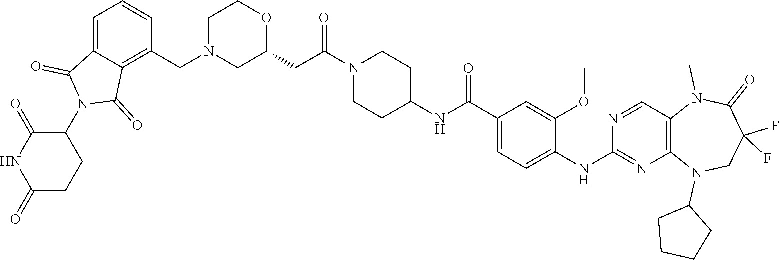 [SMILES: COc1cc(C(=O)NC2CCN(C(=O)C[C@@H]3CN(Cc4cccc5c4C(=O)N(C4CCC(=O)NC4=O)C5=O)CCO3)CC2)ccc1Nc1ncc2c(n1)N(C1CCCC1)CC(F)(F)C(=O)N2C] |
| 172 | 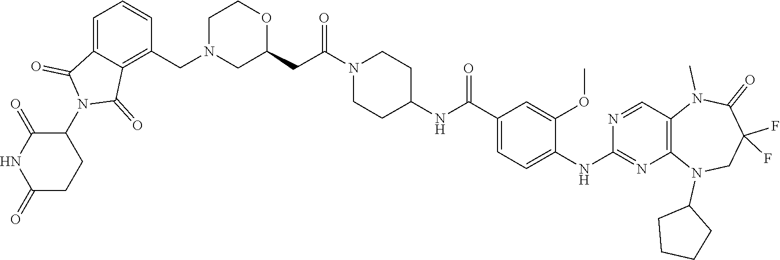 [SMILES: COc1cc(C(=O)NC2CCN(C(=O)C[C@H]3CN(Cc4cccc5c4C(=O)N(C4CCC(=O)NC4=O)C5=O)CCO3)CC2)ccc1Nc1ncc2c(n1)N(C1CCCC1)CC(F)(F)C(=O)N2C] |
| 173 | 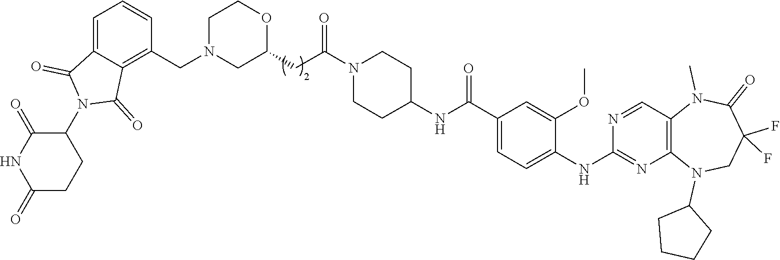 |
| 174 | 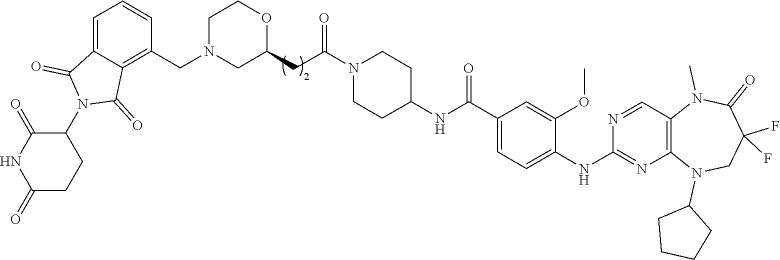 |
| 175 | 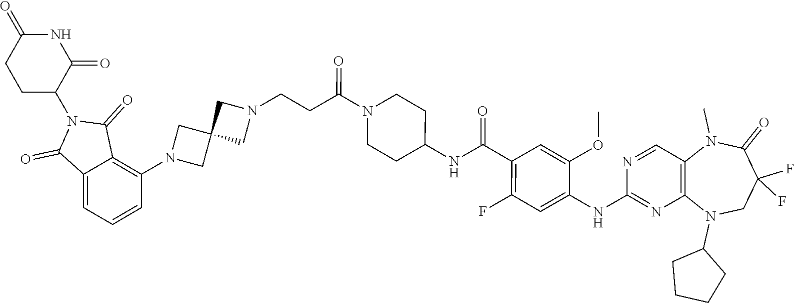 |
| 176 | 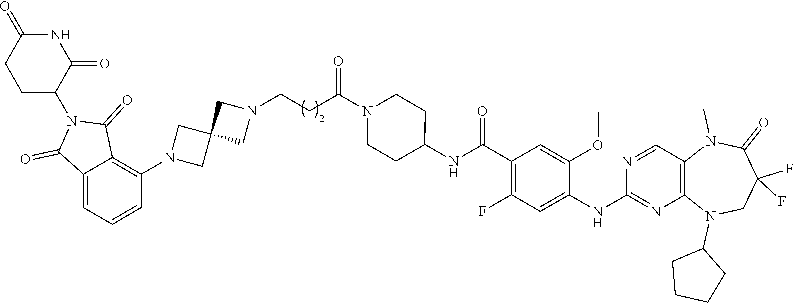 |
| 177 | 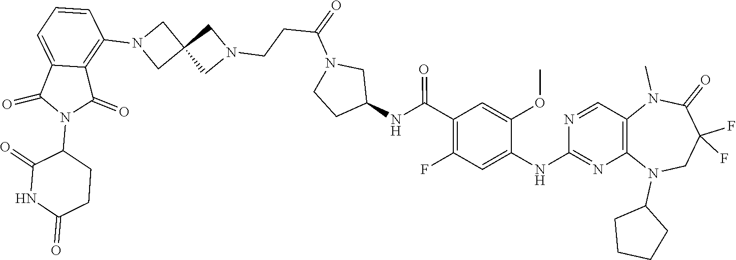 |
| 178 | 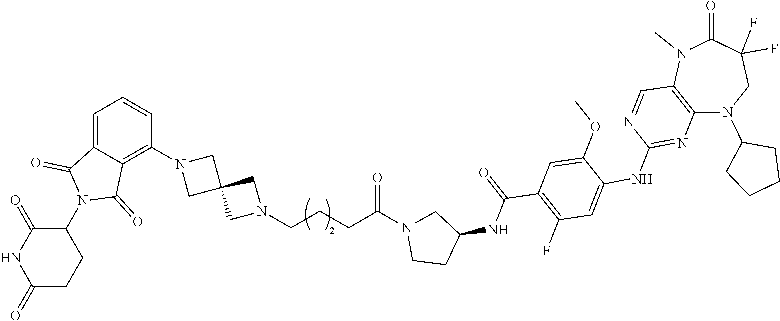 |
| 179 | 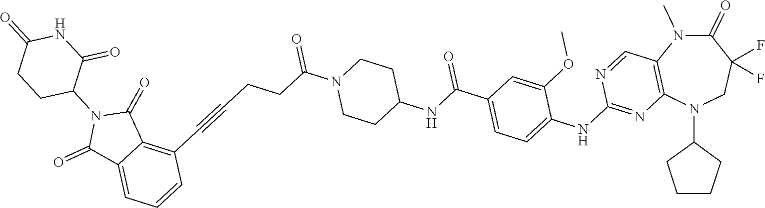 [SMILES: COc1cc(C(=O)NC2CCN(C(=O)CCC#Cc3cccc4c3C(=O)N(C3CCC(=O)NC3=O)C4=O)CC2)ccc1Nc1ncc2c(n1)N(C1CCCC1)CC(F)(F)C(=O)N2C] |
| 180 | 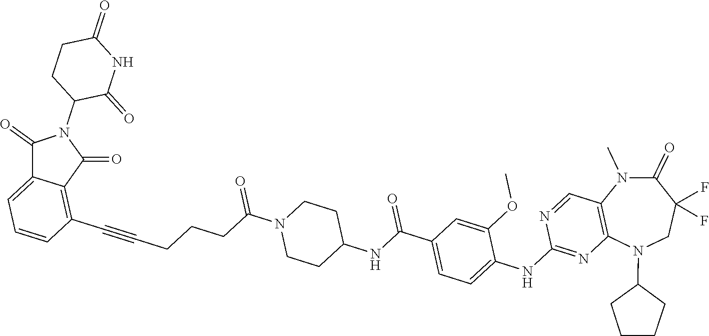 [SMILES: COc1cc(C(=O)NC2CCN(C(=O)CCCC#Cc3cccc4c3C(=O)N(C3CCC(=O)NC3=O)C4=O)CC2)ccc1Nc1ncc2c(n1)N(C1CCCC1)CC(F)(F)C(=O)N2C] |
| 181 | 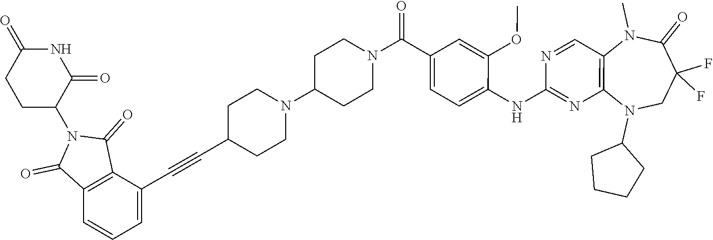 [SMILES: COc1cc(C(=O)N2CCC(N3CCC(C#Cc4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 182 | 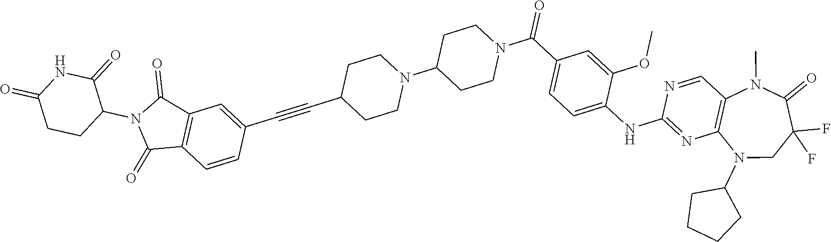 [SMILES: COc1cc(C(=O)N2CCC(N3CCC(C#Cc4ccc5c(c4)C(=O)N(C4CCC(=O)NC4=O)C5=O)CC3)CC2)ccc1Nc1ncc2c(n1)N(C1CCCC1)CC(F)(F)C(=O)N2C] |
| 183 | 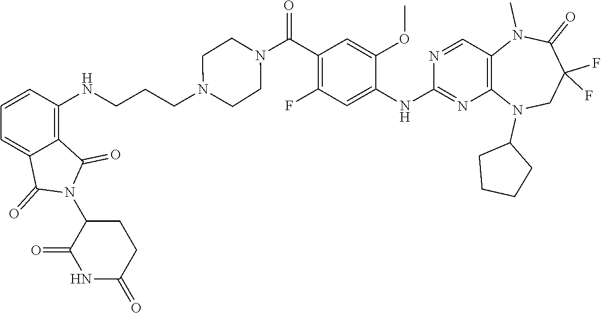 [SMILES: COc1cc(C(=O)N2CCN(CCCNc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 184 | 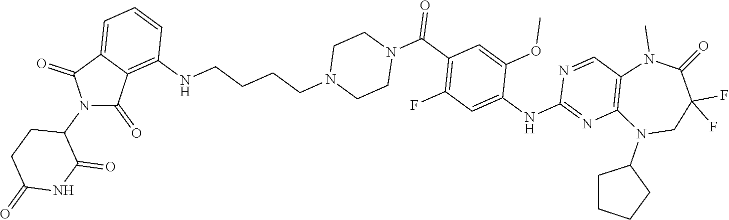 [SMILES: COc1cc(C(=O)N2CCN(CCCCNc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 185 | 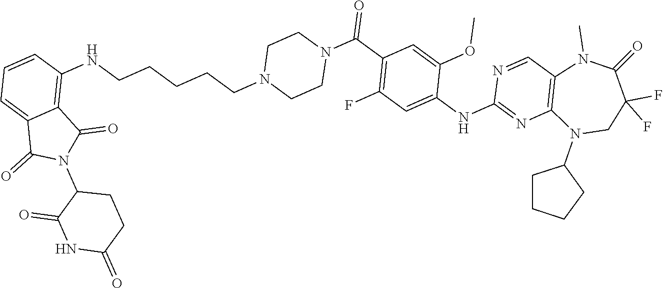 [SMILES: COc1cc(C(=O)N2CCN(CCCCCNc3cccc4c3C(=O)N(C3CCC(=O)NC3=O)C4=O)CC2)c(F)cc1Nc1ncc2c(n1)N(C1CCCC1)CC(F)(F)C(=O)N2C] |
| 186 | 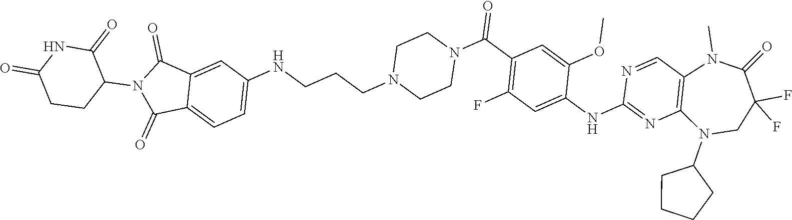 [SMILES: COc1cc(C(=O)N2CCN(CCCNc3ccc4c(c3)C(=O)N(C3CCC(=O)NC3=O)C4=O)CC2)c(F)cc1Nc1ncc2c(n1)N(C1CCCC1)CC(F)(F)C(=O)N2C] |
| 187 | 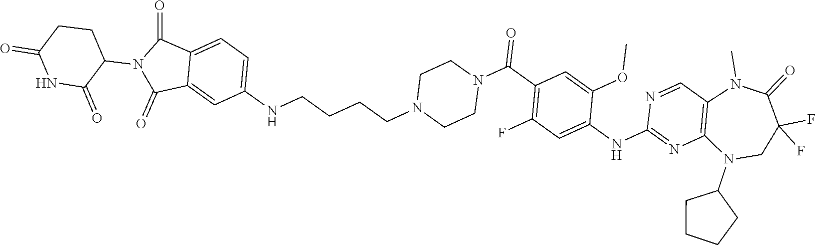 [SMILES: COc1cc(C(=O)N2CCN(CCCCNc3ccc4c(c3)C(=O)N(C3CCC(=O)NC3=O)C4=O)CC2)c(F)cc1Nc1ncc2c(n1)N(C1CCCC1)CC(F)(F)C(=O)N2C] |
| 188 | 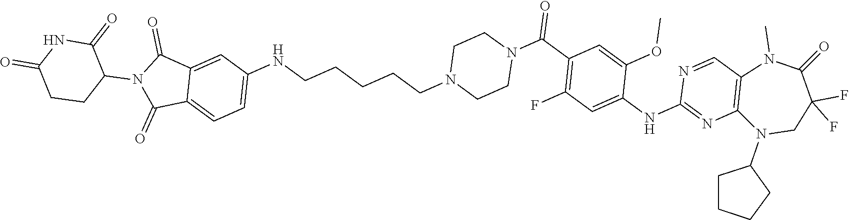 [SMILES: COc1cc(C(=O)N2CCN(CCCCCNc3ccc4c(c3)C(=O)N(C3CCC(=O)NC3=O)C4=O)CC2)c(F)cc1Nc1ncc2c(n1)N(C1CCCC1)CC(F)(F)C(=O)N2C] |
| 189 | 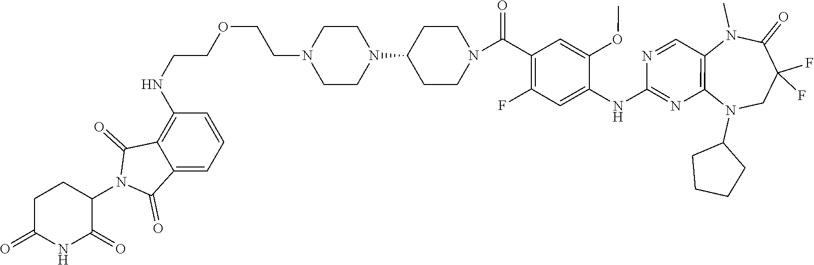 |
| 190 | 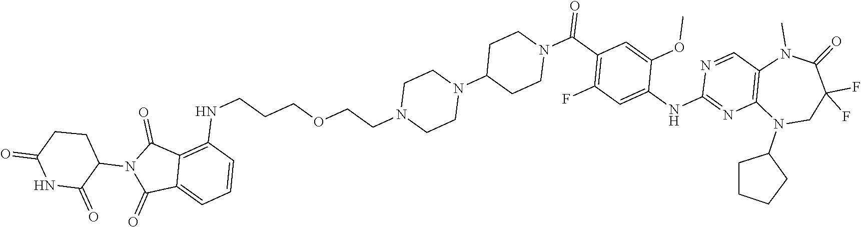 [SMILES: COc1cc(C(=O)N2CCC(N3CCN(CCOCCCNc4cccc5c4C(=O)N(C4CCC(=O)NC4=O)C5=O)CC3)CC2)c(F)cc1Nc1ncc2c(n1)N(C1CCCC1)CC(F)(F)C(=O)N2C] |
| 191 | 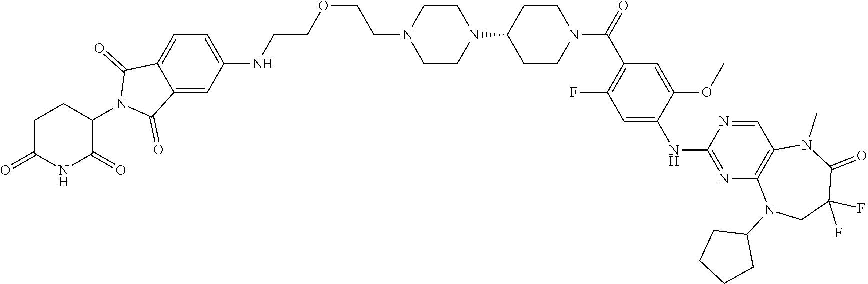 |
| 192 | 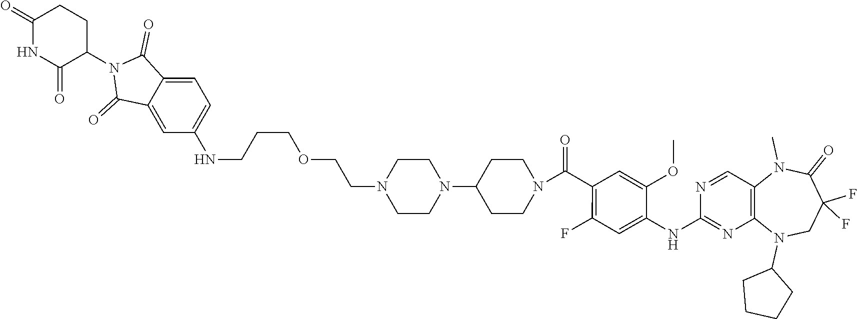 [SMILES: COc1cc(C(=O)N2CCC(N3CCN(CCOCCCNc4ccc5c(c4)C(=O)N(C4CCC(=O)NC4=O)C5=O)CC3)CC2)c(F)cc1Nc1ncc2c(n1)N(C1CCCC1)CC(F)(F)C(=O)N2C] |
| 193 | 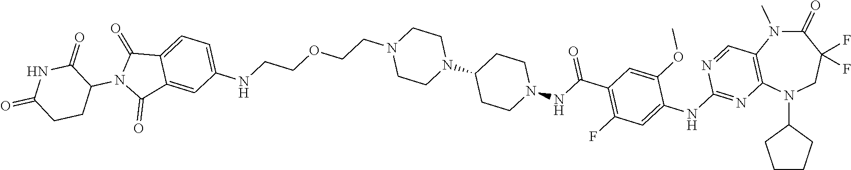 |
| 194 | 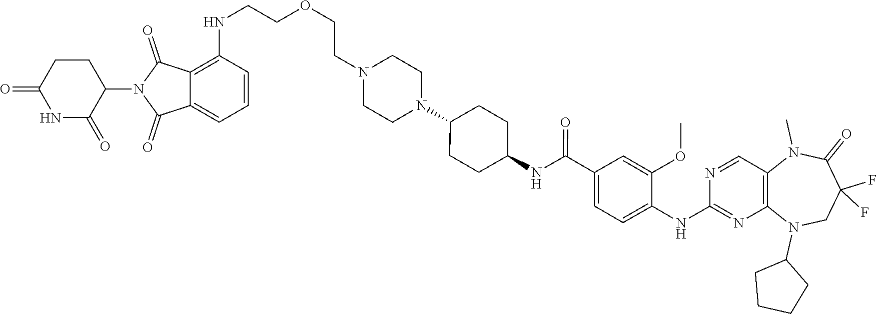 [SMILES: COc1cc(C(=O)N[C@H]2CC[C@H](N3CCN(CCOCCNc4cccc5c4C(=O)N(C4CCC(=O)NC4=O)C5=O)CC3)CC2)ccc1Nc1ncc2c(n1)N(C1CCCC1)CC(F)(F)C(=O)N2C] |
| 195 | 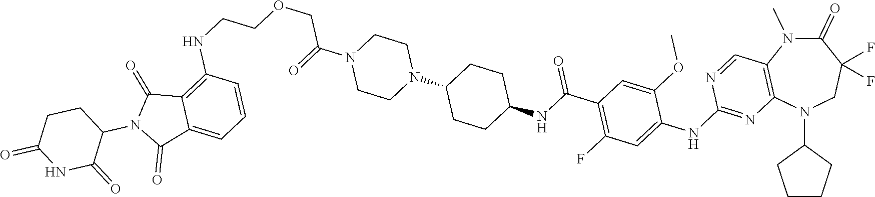 [SMILES: COc1cc(C(=O)N[C@H]2CC[C@H](N3CCN(C(=O)COCCNc4cccc5c4C(=O)N(C4CCC(=O)NC4=O)C5=O)CC3)CC2)c(F)cc1Nc1ncc2c(n1)N(C1CCCC1)CC(F)(F)C(=O)N2C] |
| 196 | 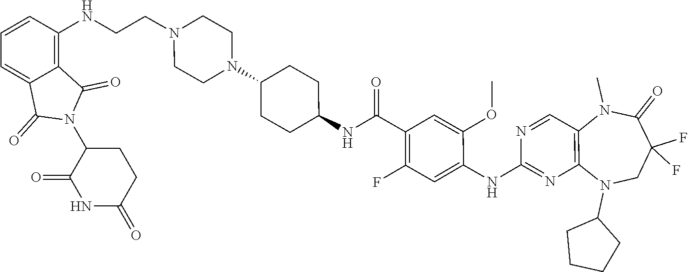 [SMILES: COc1cc(C(=O)N[C@H]2CC[C@H](N3CCN(CCNc4cccc5c4C(=O)N(C4CCC(=O)NC4=O)C5=O)CC3)CC2)c(F)cc1Nc1ncc2c(n1)N(C1CCCC1)CC(F)(F)C(=O)N2C] |
| 197 | 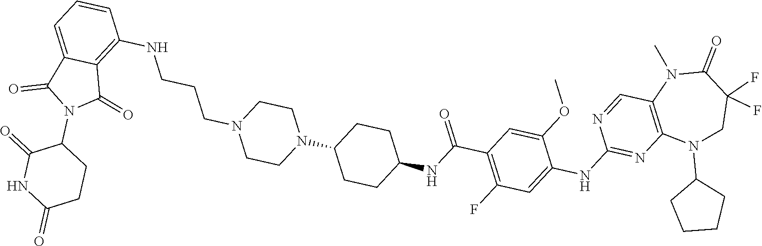 [SMILES: COc1cc(C(=O)N[C@H]2CC[C@H](N3CCN(CCCNc4cccc5c4C(=O)N(C4CCC(=O)NC4=O)C5=O)CC3)CC2)c(F)cc1Nc1ncc2c(n1)N(C1CCCC1)CC(F)(F)C(=O)N2C] |
| 198 | 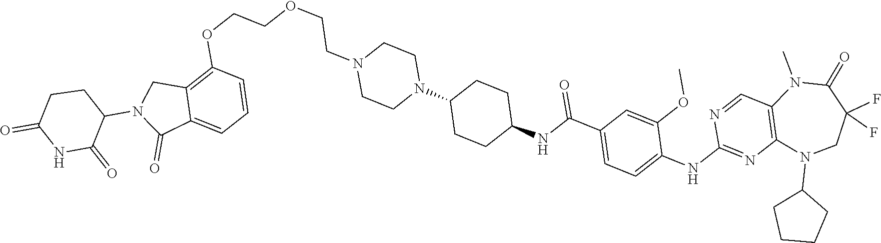 [SMILES: COc1cc(C(=O)N[C@H]2CC[C@H](N3CCN(CCOCCOc4cccc5c4CN(C4CCC(=O)NC4=O)C5=O)CC3)CC2)ccc1Nc1ncc2c(n1)N(C1CCCC1)CC(F)(F)C(=O)N2C] |
| 199 | 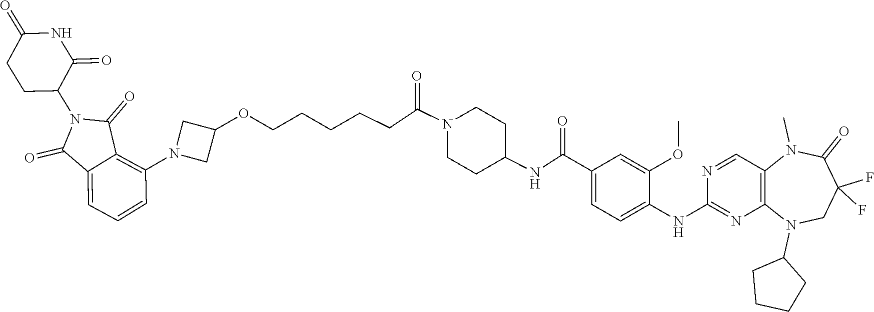 [SMILES: COc1cc(C(=O)NC2CCN(C(=O)CCCCCOC3CN(c4cccc5c4C(=O)N(C4CCC(=O)NC4=O)C5=O)C3)CC2)ccc1Nc1ncc2c(n1)N(C1CCCC1)CC(F)(F)C(=O)N2C] |
| 200 | 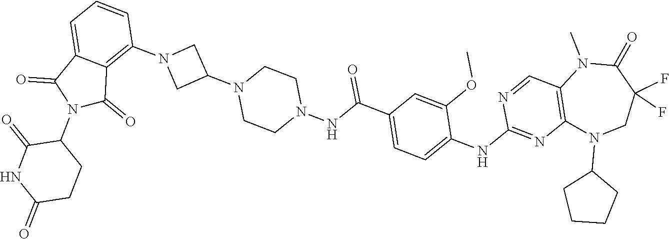 [SMILES: COc1cc(C(=O)NN2CCN(C3CN(c4cccc5c4C(=O)N(C4CCC(=O)NC4=O)C5=O)C3)CC2)ccc1Nc1ncc2c(n1)N(C1CCCC1)CC(F)(F)C(=O)N2C] |
| 201 | 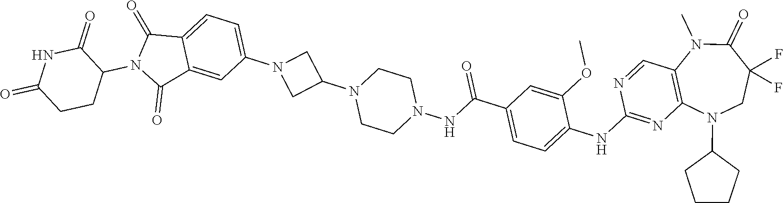 [SMILES: COc1cc(C(=O)NN2CCN(C3CN(c4ccc5c(c4)C(=O)N(C4CCC(=O)NC4=O)C5=O)C3)CC2)ccc1Nc1ncc2c(n1)N(C1CCCC1)CC(F)(F)C(=O)N2C] |
| 202 | 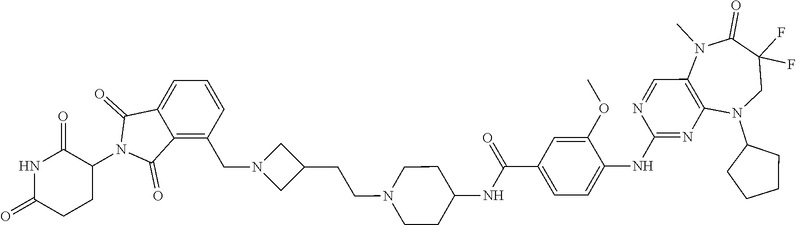 [SMILES: COc1cc(C(=O)NC2CCN(CCC3CN(Cc4cccc5c4C(=O)N(C4CCC(=O)NC4=O)C5=O)C3)CC2)ccc1Nc1ncc2c(n1)N(C1CCCC1)CC(F)(F)C(=O)N2C] |
| 203 | 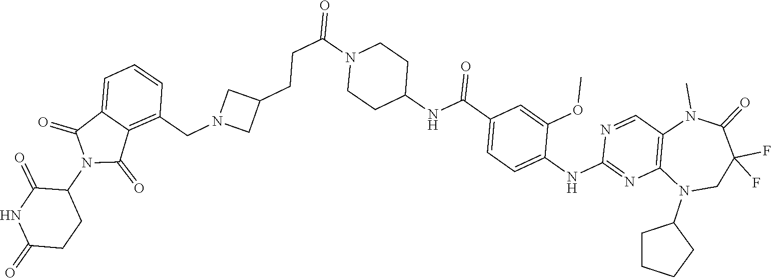 [SMILES: COc1cc(C(=O)NC2CCN(C(=O)CCC3CN(Cc4cccc5c4C(=O)N(C4CCC(=O)NC4=O)C5=O)C3)CC2)ccc1Nc1ncc2c(n1)N(C1CCCC1)CC(F)(F)C(=O)N2C] |
| 204 | 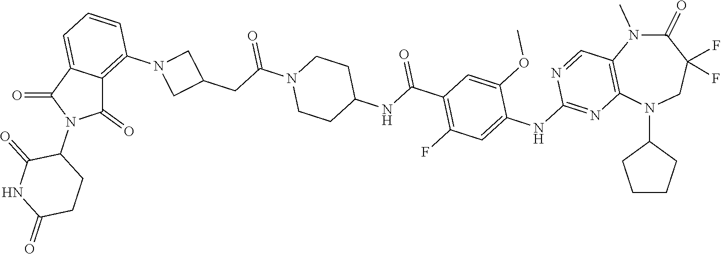 [SMILES: COc1cc(C(=O)NC2CCN(C(=O)CC3CN(c4cccc5c4C(=O)N(C4CCC(=O)NC4=O)C5=O)C3)CC2)c(F)cc1Nc1ncc2c(n1)N(C1CCCC1)CC(F)(F)C(=O)N2C] |
| 205 | 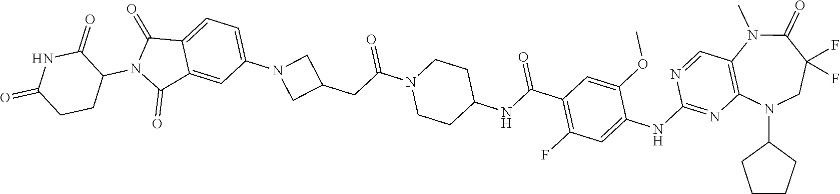 [SMILES: COc1cc(C(=O)NC2CCN(C(=O)CC3CN(c4ccc5c(c4)C(=O)N(C4CCC(=O)NC4=O)C5=O)C3)CC2)c(F)cc1Nc1ncc2c(n1)N(C1CCCC1)CC(F)(F)C(=O)N2C] |
| 206 | 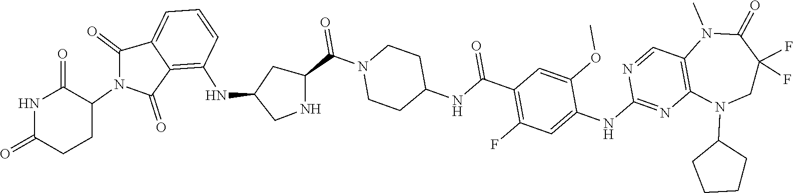 [SMILES: COc1cc(C(=O)NC2CCN(C(=O)[C@@H]3C[C@H](Nc4cccc5c4C(=O)N(C4CCC(=O)NC4=O)C5=O)CN3)CC2)c(F)cc1Nc1ncc2c(n1)N(C1CCCC1)CC(F)(F)C(=O)N2C] |
| 207 | 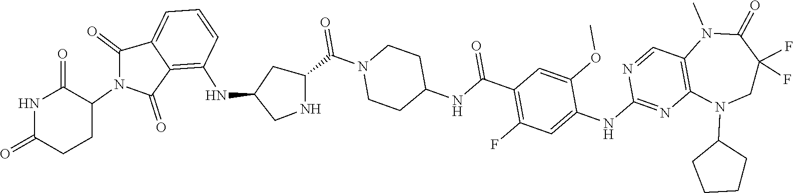 [SMILES: COc1cc(C(=O)NC2CCN(C(=O)[C@H]3C[C@H](Nc4cccc5c4C(=O)N(C4CCC(=O)NC4=O)C5=O)CN3)CC2)c(F)cc1Nc1ncc2c(n1)N(C1CCCC1)CC(F)(F)C(=O)N2C] |
| 208 | 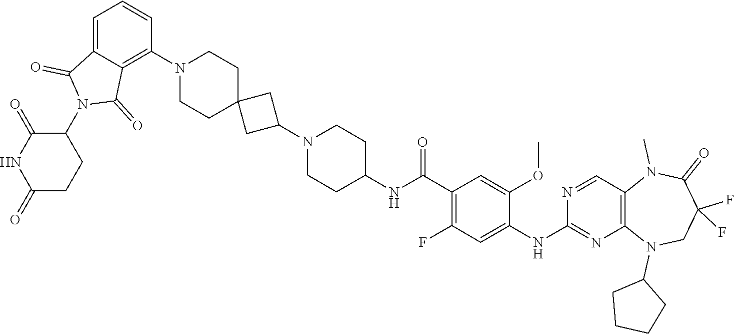 [SMILES: COc1cc(C(=O)NC2CCN(C3CC4(CCN(c5cccc6c5C(=O)N(C5CCC(=O)NC5=O)C6=O)CC4)C3)CC2)c(F)cc1Nc1ncc2c(n1)N(C1CCCC1)CC(F)(F)C(=O)N2C] |
| 209 | 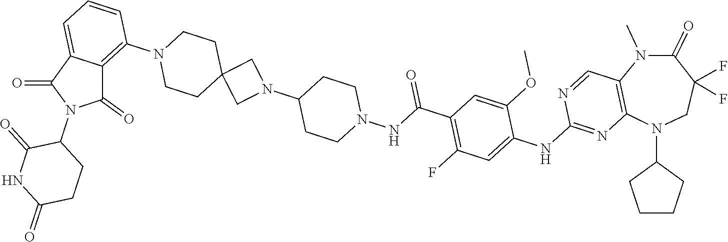 [SMILES: COc1cc(C(=O)NN2CCC(N3CC4(CCN(c5cccc6c5C(=O)N(C5CCC(=O)NC5=O)C6=O)CC4)C3)CC2)c(F)cc1Nc1ncc2c(n1)N(C1CCCC1)CC(F)(F)C(=O)N2C] |
| 210 | 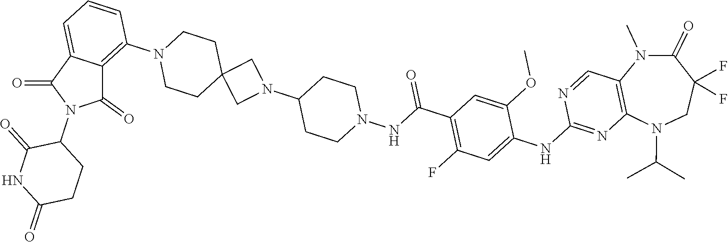 [SMILES: COc1cc(C(=O)NN2CCC(N3CC4(CCN(c5cccc6c5C(=O)N(C5CCC(=O)NC5=O)C6=O)CC4)C3)CC2)c(F)cc1Nc1ncc2c(n1)N(C(C)C)CC(F)(F)C(=O)N2C] |
| 211 | 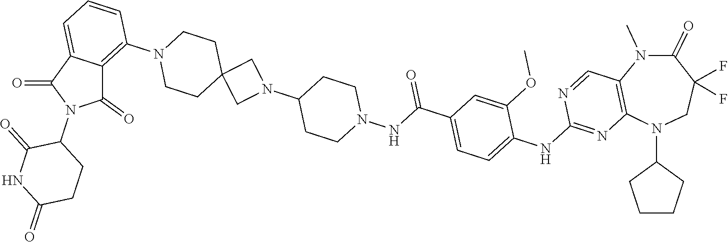 [SMILES: COc1cc(C(=O)NN2CCC(N3CC4(CCN(c5cccc6c5C(=O)N(C5CCC(=O)NC5=O)C6=O)CC4)C3)CC2)ccc1Nc1ncc2c(n1)N(C1CCCC1)CC(F)(F)C(=O)N2C] |
| 212 | 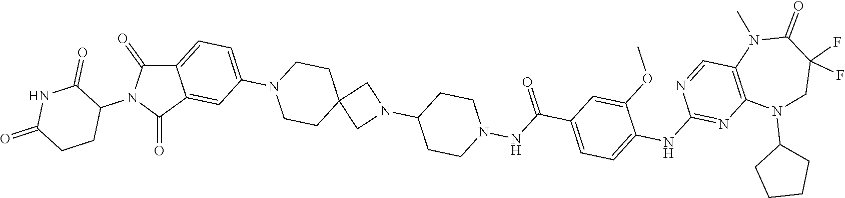 [SMILES: COc1cc(C(=O)NN2CCC(N3CC4(CCN(c5ccc6c(c5)C(=O)N(C5CCC(=O)NC5=O)C6=O)CC4)C3)CC2)ccc1Nc1ncc2c(n1)N(C1CCCC1)CC(F)(F)C(=O)N2C] |
| 213 | 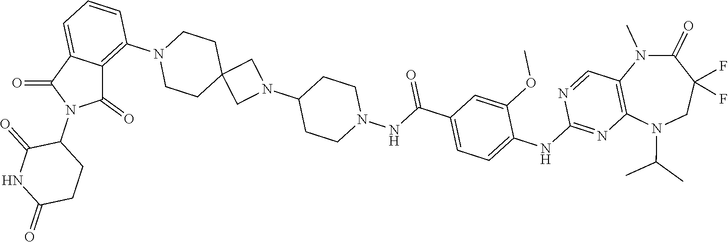 [SMILES: COc1cc(C(=O)NN2CCC(N3CC4(CCN(c5cccc6c5C(=O)N(C5CCC(=O)NC5=O)C6=O)CC4)C3)CC2)ccc1Nc1ncc2c(n1)N(C(C)C)CC(F)(F)C(=O)N2C] |
| 214 | 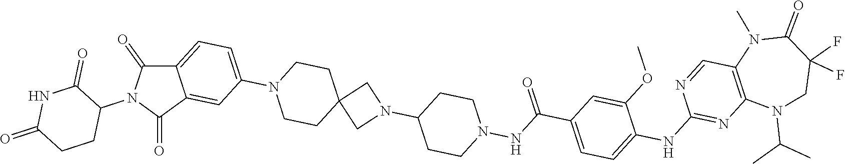 [SMILES: COc1cc(C(=O)NN2CCC(N3CC4(CCN(c5ccc6c(c5)C(=O)N(C5CCC(=O)NC5=O)C6=O)CC4)C3)CC2)ccc1Nc1ncc2c(n1)N(C(C)C)CC(F)(F)C(=O)N2C] |
| 215 | 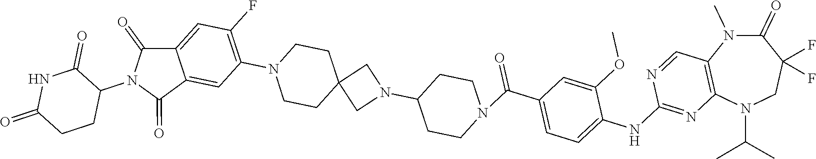 [SMILES: COc1cc(C(=O)N2CCC(N3CC4(CCN(c5cc6c(cc5F)C(=O)N(C5CCC(=O)NC5=O)C6=O)CC4)C3)CC2)ccc1Nc1ncc2c(n1)N(C(C)C)CC(F)(F)C(=O)N2C] |
| 216 | 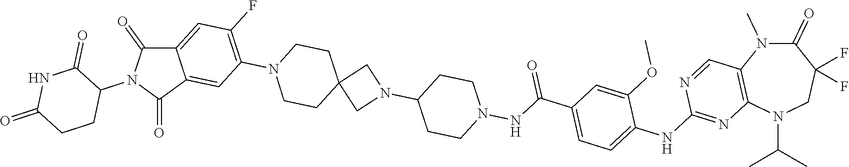 [SMILES: COc1cc(C(=O)NN2CCC(N3CC4(CCN(c5cc6c(cc5F)C(=O)N(C5CCC(=O)NC5=O)C6=O)CC4)C3)CC2)ccc1Nc1ncc2c(n1)N(C(C)C)CC(F)(F)C(=O)N2C] |
| 217 | 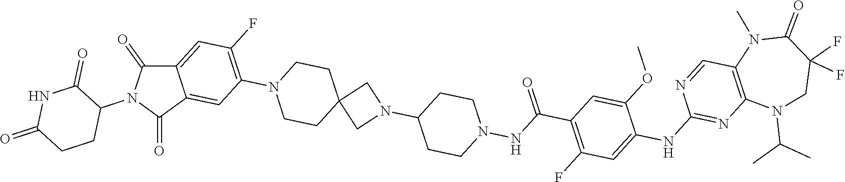 [SMILES: COc1cc(C(=O)NN2CCC(N3CC4(CCN(c5cc6c(cc5F)C(=O)N(C5CCC(=O)NC5=O)C6=O)CC4)C3)CC2)c(F)cc1Nc1ncc2c(n1)N(C(C)C)CC(F)(F)C(=O)N2C] |
| 218 | 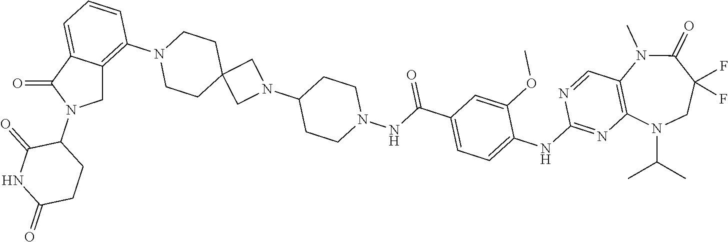 [SMILES: COc1cc(C(=O)NN2CCC(N3CC4(CCN(c5cccc6c5CN(C5CCC(=O)NC5=O)C6=O)CC4)C3)CC2)ccc1Nc1ncc2c(n1)N(C(C)C)CC(F)(F)C(=O)N2C] |
| 219 | 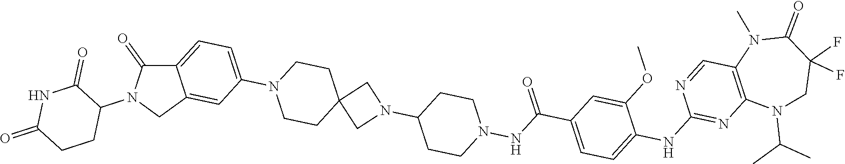 [SMILES: COc1cc(C(=O)NN2CCC(N3CC4(CCN(c5ccc6c(c5)CN(C5CCC(=O)NC5=O)C6=O)CC4)C3)CC2)ccc1Nc1ncc2c(n1)N(C(C)C)CC(F)(F)C(=O)N2C] |
| 220 | 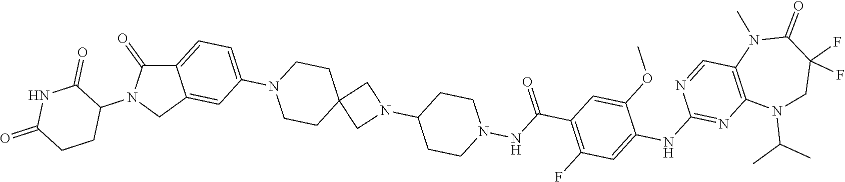 [SMILES: COc1cc(C(=O)NN2CCC(N3CC4(CCN(c5ccc6c(c5)CN(C5CCC(=O)NC5=O)C6=O)CC4)C3)CC2)c(F)cc1Nc1ncc2c(n1)N(C(C)C)CC(F)(F)C(=O)N2C] |
| 221 | 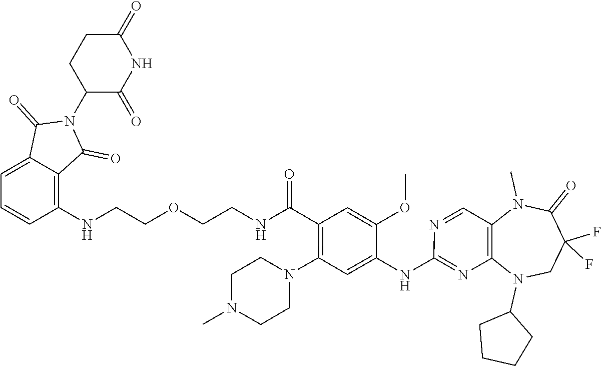 [SMILES: COc1cc(C(=O)NCCOCCNc2cccc3c2C(=O)N(C2CCC(=O)NC2=O)C3=O)c(N2CCN(C)CC2)cc1Nc1ncc2c(n1)N(C1CCCC1)CC(F)(F)C(=O)N2C] |
| 222 | 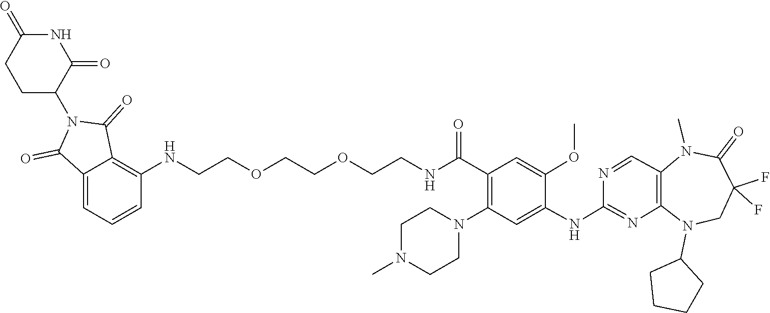 [SMILES: COc1cc(C(=O)NCCOCCOCCNc2cccc3c2C(=O)N(C2CCC(=O)NC2=O)C3=O)c(N2CCN(C)CC2)cc1Nc1ncc2c(n1)N(C1CCCC1)CC(F)(F)C(=O)N2C] |
| 223 | 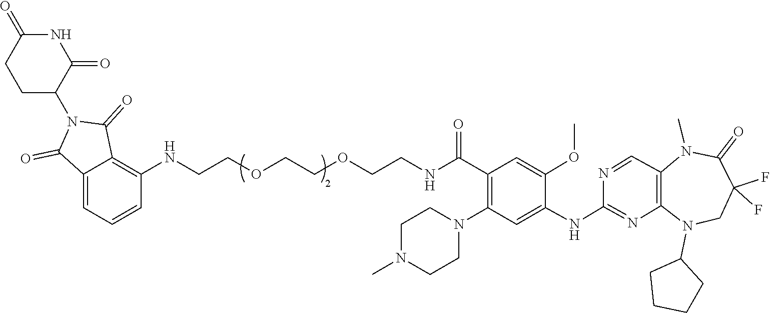 |
| 224 | 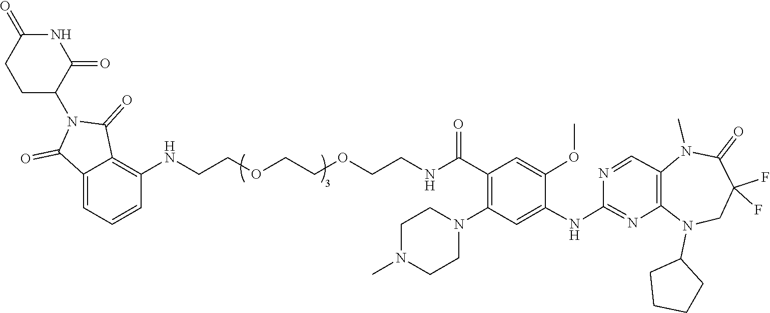 |
| 225 | 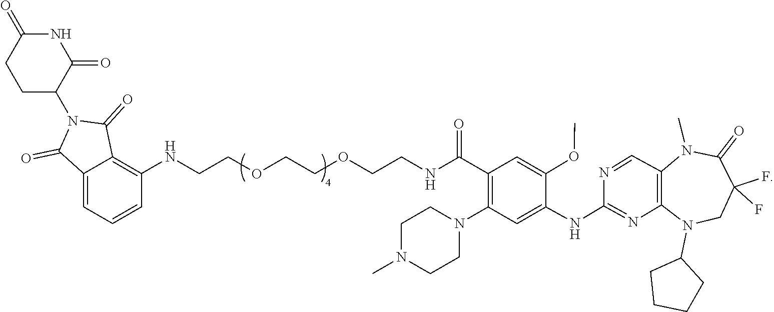 |

8. A pharmaceutical composition comprising the compound, the stereoisomer thereof or the pharmaceutically acceptable salt thereof according to claim 1.

9. A pharmaceutical composition for preventing or treating PLK1-related disease comprising the compound, the stereoisomer thereof or the pharmaceutically acceptable salt thereof according claim 1.

10. The pharmaceutical composition according to claim 9, wherein the PLK1-related disease is one or more selected from cancer, benign tumor or neurological disorder.

11. The pharmaceutical composition according to claim 10, wherein the cancer or benign tumor is one or more selected from squamous cell carcinoma, small cell lung cancer, non-small cell lung cancer, lung adenocarcinoma, lung squamous cell carcinoma, peritoneal cancer, skin cancer, skin or intraocular melanoma, rectal cancer, anal muscle cancer, esophageal cancer, small intestine cancer, endocrine cancer, parathyroid cancer, adrenal cancer, soft tissue sarcoma, urethral cancer, chronic or acute leukemia, lymphocytic lymphoma, hepatocellular carcinoma, gastrointestinal cancer, gastric cancer, pancreatic cancer, glioblastoma, cervical cancer, ovarian cancer, liver cancer, bladder cancer, liver tumor, breast cancer, colon cancer, colorectal cancer, endometrial or uterine cancer, salivary gland cancer, kidney cancer, prostate cancer, vulvar cancer, thyroid cancer, head and neck cancer, brain cancer, osteosarcoma, Barrett's esophagus, colon adenoma and polyp, breast fibroadenoma and cyst, monoclonal gammopathy of undetermined significance (MGUS), monoclonal lymphocytosis, solid tumor, blood cancer, bone cancer, large cell lymphoma, adrenocorticoid tumor, t cell lymphoma/leukemia, neuroendocrine cancer, neuroendocrine tumor, cholangiocarcinoma, neuroblastoma, glioblastoma, and/or glioma.

12. The pharmaceutical composition according to claim 10, wherein the neurological disorder is a central nervous system disease, a neurodegenerative disease, Alzheimer's disease, Parkinson's disease, multiple sclerosis, Huntington's disease, senile dementia, epilepsy, Lou Gehrig, stroke, or nerve damage and axonal degeneration-related disorders following brain or spinal cord injury.

13. A method for treating or preventing PLK1-related disease, comprising administering to the subject in need thereof a therapeutically effective amount of the compound, the stereoisomer thereof or the pharmaceutically acceptable salt thereof according to claim 1.

14. The method for treating or preventing PLK1-related disease according to claim 13, wherein the compound, the stereoisomer thereof or the pharmaceutically acceptable salt thereof induces degradation for PLK1 protein.

15-16. (canceled)
